(19) United States
(12) Patent Application Publication
(10) Pub. No.: US 2026/0002177 A1
(43) Pub. Date: Jan. 1, 2026

(54) HIGH THROUGHPUT ENGINEERING OF FUNCTIONAL AAV CAPSIDS

(71) Applicant: Shape Therapeutics Inc., Seattle, WA (US)

(72) Inventors: Francois VIGNEAULT, Clyde Hill, WA (US); Thomas PACKARD, Seattle, WA (US); David Jeffrey HUSS, Seattle, WA (US); Kevin Christopher STEIN, Kenmore, WA (US)

(21) Appl. No.: 19/027,134

(22) Filed: Jan. 17, 2025

Related U.S. Application Data
(63) Continuation of application No. 18/755,111, filed on Jun. 26, 2024, now abandoned.
Application No. 18/755,111 is a continuation of application No. 18/542,426, filed on Dec. 15, 2023, now abandoned.
Application No. 18/542,426 is a continuation of application No. 18/137,343, filed on Apr. 20, 2023, now abandoned.
Application No. 18/137,343 is a continuation of application No. 17/331,462, filed on May 26, 2021, now abandoned.
(60) Provisional application No. 63/030,038, filed on May 26, 2020.
(60) Provisional application No. 63/119,554, filed on Nov. 30, 2020.
(60) Provisional application No. 63/134,885, filed on Jan. 7, 2021.
(60) Provisional application No. 63/181,037, filed on Apr. 28, 2021.

(51) Int. Cl. C12N 15/86; C40B 40/02

(52) U.S. Cl. CPC C12N 15/86; C40B 40/02; C12N 2750/14111

(57) ABSTRACT

Disclosed herein are engineered AAV VP capsid polypeptides with the ability to assemble into virus particles and having improved tissue tropism to, for example, CNS tissues. The capsids are engineered using the high throughput discovery system described herein. In certain embodiments, provided herein are recombinant adeno-associated virus (AAV) VP capsid polypeptides having at least one mutation in a residue corresponding to residue 581 to residue 589 in SEQ ID NO: 1.

### 1. CROSS-REFERENCE TO RELATED APPLICATIONS

This application is a continuation of U.S. application Ser. No. 18/755,111, filed Jun. 26, 2024, which is a continuation of U.S. application Ser. No. 18/542,426, filed Dec. 15, 2023, now abandoned, which is a continuation of U.S. application Ser. No. 18/137,343, filed Apr. 20, 2023, now abandoned, which is a continuation of U.S. application Ser. No. 17/331,462, filed May 26, 2021, now abandoned, which claims the benefit of and priority to U.S. Provisional Patent Application No. 63/030,038 filed on May 26, 2020; U.S. Provisional Patent Application No. 63/119,554 filed on Nov. 30, 2020; U.S. Provisional Patent Application No. 63/134,885 filed on Jan. 7, 2021; and U.S. Provisional Patent Application No. 63/181,037 filed Apr. 28, 2021, which are incorporated by reference in their entirety.

### 2. SEQUENCE LISTING

The instant application contains a Sequence Listing which has been submitted electronically in extensible Markup Language (XML file) format and is hereby incorporated herein by reference in its entirety. Said XML copy, created on Apr. 28, 2023, is named 421688_706051_SL.xml, and is 60,849,405 bytes in size.

### 3. BACKGROUND

Recombinant adeno-associated viruses (rAAV) provide the leading platform for in vivo delivery of gene therapies. Current clinical trials employ a limited number of AAV capsids, primarily from naturally occurring human or primate serotypes such as AAV1, AAV2, AAV5, AAV6, AAV8, AAV9, AAVrh.10, AAV4rh.74, and AAVhu.67. These capsids often provide suboptimal targeting to tissues of interest, both due to poor infectivity of the tissue of interest and competing liver tropism. Increasing the dose to ensure infection of desired tissues can lead to dose-dependent liver toxicity. In addition, use of naturally-occurring capsids presents an immunological memory challenge—pre-immune patient populations are excluded from treatment and repeat dosing in a previously immune naïve patient is often not possible. Thus, there is a need for additional AAV capsids for use in gene therapy, in particular capsids that confer upon the rAAV high infectivity for specific tissues and low liver tropism.

### 4. SUMMARY OF THE INVENTION

We have designed a system for high throughput engineering of functional AAV capsids with altered tropism for various tissues, and using this system have identified capsid variants that have either increased or reduced liver tropism, and increased tropism for target tissues, such as liver or central nervous system (CNS) tissues.

Provided herein is an engineered adeno-associated virus (AAV) viral protein (VP) capsid polypeptide having an amino acid sequence at least 70% identical to SEQ ID NO: 1, wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive, wherein the engineered AAV VP capsid polypeptide is capable of assembling into a recombinant AAV virion (rAAV), wherein the at least one mutation confers higher tropism for a central nervous system (CNS) tissue on the rAAV as compared to an rAAV virion having a wildtype AAV5 VP capsid polypeptide of SEQ ID NO: 1, and wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. In certain embodiments, the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid.

Additionally, provided herein is an engineered adeno-associated virus (AAV) viral protein (VP) capsid polypeptide having an amino acid sequence of SEQ ID NO: 2, wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V, wherein the engineered AAV VP capsid polypeptide is capable of assembling into a recombinant AAV virion (rAAV), and wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. In certain embodiments, the rAAV has higher tropism for a central nervous system (CNS) tissue as compared to an rAAV virion having a wildtype AAV5 VP capsid polypeptide of SEQ ID NO: 1.

### 5. BRIEF DESCRIPTION OF THE DRAWINGS

These and other features, aspects, and advantages of the present invention will become better understood with regard to the following description, and accompanying drawings where:

FIG. 1 is a schematic of the high throughput AAV capsid engineering system.

FIG. 2A provides a side view (top panel) and top view (bottom panel) of key residues of known AAV capsids that have been shown to interact with target cells. FIG. 2B illustrates salient features of the AAV capsid library described in Example 1, showing the region of introduced diversity, with residue numbering corresponding to the numbering of amino acids in AAV5 VP1.

FIG. 3 is a gel photograph showing effective removal of the linear insert and linear plasmid backbone (“BB”) following digestion with PS-DNAse, enriching “relaxed form” (“RF”) circular species for highly efficient subsequent bacterial transformation.

FIG. 4 is a schematic of the high throughput AAV capsid engineering system. The column graph on the left shows the size of the AAV capsid library at various stages of preparation, tracking the size of the library from theoretical diversity, synthesized capsid genes, total cloned variants, to sequenced assembled viruses (error bars denote minimum/maximum predicted diversity from NGS sequencing analysis). The pooled viral library is injected into non-human primates and following a period of time sufficient to ensure stable transduction, the animals are euthanized, and tissues are harvested. DNA is purified from the tissues (1), and (2) a unique molecular identifier (UMI) is appended. Exonuclease I is added to digest excess UMI-containing primers (3). Subsequent PCR amplification adds sequencing indexes/tissue barcoding and adapters for next generation sequencing (NGS) sequencing. The addition of UMIs allows for high resolution frequency analysis of capsid variants in the tissues.

FIGS. 5A-5C illustrates the packaging approach used to maximize rAAV production from the library, with FIG. 5A comparing standard triple transfection (top panel) to the two-plasmid cis packaging approach used herein (bottom panel), and FIG. 5B showing relative production of wild type AAV5 using the two transfection approaches. FIG. 5C is an example of UMI distribution in a liver specimen and shows a majority of capsid variants are found a single time (single UMI). However, a subset of capsid variants is enriched in a given tissue and has increased numbers of UMIs, corresponding to their increased frequency in the target tissue.

FIGS. 6A-6B are heat maps and a statistical analysis showing clear functional selection through the stages of viral assembly and tissue transduction. FIG. 6A is a heat map prepared at various stages of the high throughput system from pre-assembly, assembled virus, to liver transduction. For the pre-assembly heat map, the library of capsid variants was cloned into plasmids and transformed into electrocompetent bacteria. The resultant library was sequenced and amino acid diversity was measured at each position, with the low levels of positional variation likely arising from synthesis error. Subsequent assembly into virus shows clear amino acid residue/positional biases that highlight selection for viral assembly. Liver transducing viruses show even greater patterns of AA residue/positional selection, with distinctly favored/disfavored variants. Note that each of these heatmaps is normalized by their respective input frequencies. FIG. 6B is a table of statistical analysis (ANOVA) showing that in three liver samples, amino acid residue distribution and residue-position vary significantly, but inter-sample variation is not significant.

FIGS. 7A-7B show analysis of repeat observed (high UMI) capsid variants between multiple samples. FIG. 7A is a Venn diagram illustrating counts of overlapping variant identification in two liver samples: ˜38% of variants with >10 UMIs were observed in both samples. FIG. 7B is a heat map showing positional amino acid distribution illustrating the strongly selected residues/positions of repeat observed capsid variants.

FIG. 8 is a schematic of machine learning-based clustering of capsid variants. The example utilizes capsid variant sequences upon which machine learning algorithms were used to map the similarity of capsids among those that infected the liver. This output can then be used to inform selection of candidate variants to selectively target the tissue of interest.

FIG. 9 illustrates an example of the performance of the library as a whole infecting cortex at a higher relative level than liver after the intravenous administration to a non-human primate (NHP). DNA was isolated from the liver and cortex and either qPCR (at left) or droplet digital PCR (ddPCR) (at right) were used to quantify viral genomes recovered from the respective tissues. By these methods of quantification, the library as a whole has ˜2-fold increased CNS targeting over the liver.

FIG. 10 illustrates wild-type crystal structure of AAV5 capsid emphasizing electrostatic potential, and two exemplary recombinant VP1 capsid variants obtained by the methods described herein.

FIGS. 11A-D show Venn diagrams and analytic tables depicting the number of unique sequence variants found in liver tissue only, liver and brain cortex tissues, and cortex tissue only for three different sequencing analysis filters. Next-generation sequencing was performed on viral genomic capsid variants recovered from liver or cortical tissue samples. Unique Molecular Identifiers (UMIs) were appended as part of the molecular recovery process as described herein. This allows for increased confidence that a given capsid variant is present in the tissue, and may be correlated to abundance. FIG. 11A shows a Venn diagram in which the sequencing analysis filter applied was 4 or greater distinct UMIs (also referred to herein as “count”), per each distinct capsid sequence variant. FIG. 11B shows a Venn diagram in which the sequencing analysis filter applied was 50 or greater UMIs. FIG. 11C shows a Venn diagram in which the sequencing analysis filter applied was 100 or greater distinct UMIs. FIG. 11D shows a histogram analysis of the distribution of UMIs in the population of capsid sequence variants found in cortex only in which the sequencing analysis filter applied was 50 or greater UMIs as shown in FIG. 11B.

FIG. 12 shows heatmaps of normalized amino acid residue frequency in the rAAV5 capsid polypeptide sequences of the present disclosure and how they are enriched moving from pre-assembly to post-assembly to CNS-transducing subsets of variants. The x-axes of the heatmaps indicate the position in the 581-589 region and the y-axis includes all amino acid residues.

FIG. 13 shows an example of machine learning (ML) model performance validation. Distinct variants are found in CNS and non-CNS tissues, as well as some shared variants found in both (left). Random Forest (RF) ML models show good performance at predicting CNS targeting. At high predicted class probability values, the ML model can resolve CNS-targeting from non-CNS targeting variant sequences (middle, right).

FIG. 14 illustrates top 20 positional features contributing to model output probability (at left). Shapley Additive Explanations (SHAP) values can be used to interrogate the relative contribution of features to model predictions. These features can be further compared between tissue targeting and non-targeting variants. At right is a model of a CNS variant having a sequence of KRLQQMETM (SEQ ID NO: 1117), representing some features predicted to increase CNS-targeting.

FIG. 15 shows recovery of rAAV5 variants of the present disclosure from two NHPs across all tissue types including skin, liver, lung, heart, spleen, lymph node, thyroid gland, skeletal muscle, bone marrow, mammary gland, adrenal gland, colon, sciatic nerve (a peripheral nerve), and a number of CNS tissues (forebrain cortex, occipital cortex, temporal cortex, thalamus, hypothalamus, substantia nigra, hippocampus DG, hippocampus CA1, hippocampus CA3, cerebellum, and spinal cord). Analysis of tissue samples enabled the ability to enrich favored properties of a particular variant (e.g., enriched in a first tissue and not enriched in one or more other tissues). Machine learning algorithms were applied to discover determinants of tissue tropism.

FIG. 16 shows that rAAV5 variants include variants present in the CNS at large, as well as found in the substantia nigra, a subregion of the brain particularly affected in Parkinson's disease. Additionally, FIG. 16 shows that machine learning models exhibited good performance at predicting determinants of high CNS specificity (at right).

FIG. 17 shows that rAAV5 variants include variants present in muscle tissue, including variants that target heart (cardiac tissue) and skeletal tissue while de-targeted to liver tissue (at left). Additionally, FIG. 17 shows that machine learning models exhibited good performance at predicting determinants of liver detargeting (at right).

FIG. 18A shows a heatmap of amino acid positional frequencies in a CNS tissue compared to all other analyzed tissues. FIG. 18B shows a heatmap of amino acid positional frequencies in a liver tissue compared to all other analyzed tissues. FIG. 18C shows a heatmap of amino acid positional frequencies in a skeletal muscle tissue compared to all other analyzed tissues. FIG. 18D shows a heatmap of amino acid positional frequencies in a heart tissue compared to all other analyzed tissues. FIG. 18E shows a heatmap of amino acid positional frequencies in a lung tissue compared to all other analyzed tissues. FIG. 18F shows a heatmap of amino acid positional frequencies in a spleen tissue compared to all other analyzed tissues. FIG. 18G shows a heatmap of amino acid positional frequencies in a lymph node tissue compared to all other analyzed tissues. FIG. 18H shows a heatmap of amino acid positional frequencies in a bone marrow tissue compared to all other analyzed tissues. FIG. 18I shows a heatmap of amino acid positional frequencies in a mammary gland tissue compared to all other analyzed tissues. FIG. 18J shows a heatmap of amino acid positional frequencies in a skin tissue compared to all other analyzed tissues. FIG. 18K shows a heatmap of amino acid positional frequencies in an adrenal gland tissue compared to all other analyzed tissues. FIG. 18L shows a heatmap of amino acid positional frequencies in a thyroid tissue compared to all other analyzed tissues. FIG. 18M shows a heatmap of amino acid positional frequencies in a colon tissue compared to all other analyzed tissues. FIG. 18N shows a heatmap of amino acid positional frequencies in a sciatic nerve tissue compared to all other analyzed tissues. FIG. 18O shows a heatmap of amino acid positional frequencies in a spinal cord tissue compared to all other analyzed tissues.

FIG. 19A shows the results at various steps of bioinformatics pre-processing of the NGS data. Stepwise read count for 48 individual samples through filters is shown. FIG. 19B shows a positional comparison of CNS/non-CNS variant sequences.

FIG. 20A shows an example of peripheral and CNS tissue samples analyzed by the methods described herein. FIG. 20B shows amino acid positional abundance in the top 1000 machine learning predicted/filtered variants recovered from the CNS compared to non-CNS variants.

FIG. 21A shows an example of an ensemble of both ML models: Random Forest (RF) and Histogram-based Gradient Boosting Tree (HGB) for averaged predicted CNS probability. Outputs of CNS-targeting probability from both ML models showed good concordance. FIG. 21B shows the distribution of average CNS-targeting probability for all CNS variants.

FIG. 22 shows an example of machine learning model performance validation. Both Histogram-based Gradient Boosting Tree (HGB) (top) and Random Forest (RF) (bottom) machine learning models showed good performance at predicting CNS targeting. At high predicted class probability values, both machine learning models resolved CNS-targeting from non-CNS targeting.

FIG. 23 shows how a set of top 20 positional features contributed to model output probability. Shapley Additive Explanations (SHAP) values were used to interrogate the relative contribution of features to model predictions. These features can be further compared between tissue targeting and non-targeting variants (as in FIGS. 24A-C).

FIGS. 24A, 24B, and 24C shows a comparison of a set of top predictive features positionally. Features were selected if they were found to be important to both the HGB & RF models. Summaries are shown of the features in the top 1000 machine learning-predicted CNS variants compared to a random 2% of CNS variants.

FIG. 25 shows a set of top 20 positional features contributing to model output probability in a machine learning analysis of sequences that target liver tissue.

FIGS. 26A, 26B, and 26C show a comparison of top predictive features positionally in a machine learning analysis of sequences that target liver tissue. Features were compared between tissue targeting and non-targeting variants.

FIG. 27 shows a set of top 20 positional features contributing to model output probability in a machine learning analysis of sequences that are liver-detargeted.

FIGS. 28A and 28B show a comparison of top predictive features positionally for liver-detargeted variants.

FIG. 29 shows a set of top 20 positional features contributing to model output probability in a machine learning analysis of sequences that target muscle tissue.

FIGS. 30A-30B show a comparison of top predictive features positionally in a machine learning analysis of sequences that target muscle tissue. Features were compared between tissue targeting and non-targeting variants.

### 6. DETAILED DESCRIPTION OF THE INVENTION

Unless described otherwise, all technical and scientific terms used herein have the meaning commonly understood by one of ordinary skill in the art to which the invention pertains.

Unless otherwise stated, whenever a range is recited, the range is inclusive of the recited endpoints. For example, the region from amino acid residue 581 to amino acid residue 589 of SEQ ID NO: 1 includes amino acid residues 581 and 589.

“Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed. The term percent “identity” or percent “homology,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. For purposes herein, percent identity and sequence similarity is performed using the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

In some cases, the identity between a reference sequence (query sequence, e.g., a sequence of the disclosure) and a subject sequence, also referred to as a global sequence alignment, can be determined using the FASTDB computer program. In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

As used herein, “tropism” of a rAAV for a tissue is defined as the ability of a given rAAV to preferentially infect a given cell or tissue. Altered or engineered tropism includes increased or decreased targeting ability for desired tissues, with a corresponding increased or decreased infection of the target tissue.

For simplicity throughout this disclosure, viral capsid protein is generally referred to as “VP.” Viral capsid protein is referred to as VP1 when referencing AAV5 VP1 positional notation. In all cases, viral capsid sequences and mutations disclosed herein should be understood as pertaining to all isoforms of the capsid protein (VP1, VP2, and VP3), as a mixture of these isoforms assemble to form virions. The positional amino acid residue designations “581-589” are relative to the translational start of the VP1 polypeptide and should be adjusted accordingly to the relative start sites of VP2 and VP3. It should be understood that the present disclosure, when describing any particular VP1 sequence with mutations at particular amino acid residue positions, necessarily also encompasses corresponding mutations in VP2 and VP3. For example, any consensus sequence or specific sequence of a VP1 capsid protein having one or more mutations in the amino acid residue s of the 581-589 region also encompasses VP2 and VP3 capsid proteins having said one or more mutations in an amino acid residue region in VP2 and VP3 corresponding to the amino acid residues of the VP1 581-589 region. For example, the amino acid residues of the 581 to 589 region of VP1 (SEQ ID NO: 1) correspond to the amino acid residues of the 445 to 453 region of VP2 (SEQ ID NO: 1115) and to the amino acid residues of 389 to 397 region of VP3 (SEQ ID NO: 1116).

It should be understood that the present disclosure includes polynucleotide sequences encoding for any sequence disclosed herein. For example, if an amino acid sequence is provided, the present disclosure also encompasses a polynucleotide sequence encoding for said amino acid sequence.

It should be understood that further embodiments include mutations in VP1, VP2, VP3, or any combination thereof that do not alter the desired properties (e.g., a particular tissue tropism) or affect viral assembly, as described herein.

As used herein, “tissue tropism” refers to a preference of a virus having an engineered VP capsid polypeptide of the present disclosure to infect a given tissue or be enriched in or accumulate in a given tissue. Tissue tropism, when used as a relative term and depending on the context in which it is described herein, refers to an increase or decrease in tissue tropism of a given rAAV virion having a first capsid polypeptide in a first tissue as compared to a second tissue and/or refers to an increase or decrease in tissue tropism of a given rAAV virion having a first capsid polypeptide to an rAAV virion having a second capsid polypeptide. In some embodiments, the first tissue can be a group of tissues. In some embodiments, the second tissue can be a group of tissues. For example, the first tissue may be CNS tissues, which comprise cortex forebrain, cortex occipital, cortex temporal, thalamus, hypothalamus, substantia nigra, hippocampus DG, hippocampus CA1, hippocampus CA3, and cerebellum and the second tissue may be a non-CNS tissue consisting collectively of liver, skeletal muscle, heart, lung, spleen, lymph node, bone marrow, mammary gland, skin, adrenal gland, thyroid, colon, sciatic nerve, and spinal cord tissues. As another example, the first tissue may be liver tissue and the second tissue may be non-liver tissue consisting collectively of CNS tissues, skeletal muscle, heart, lung, spleen, lymph node, bone marrow, mammary gland, skin, adrenal gland, thyroid, colon, sciatic nerve, and spinal cord tissues.

As used herein, the word “recombinant” in the context of an AAV capsid polypeptide, interchangeably refers to an “engineered” or “variant” AAV capsid polypeptide. As used herein, the word “recombinant” in the context of an AAV virion, abbreviated to rAAV, refers to a recombinant virus particle. Said rAAV virion is made of a capsid that may include the engineered AAV5 VP capsid polypeptides disclosed herein.

### 6.1. Capsid Engineering Methods

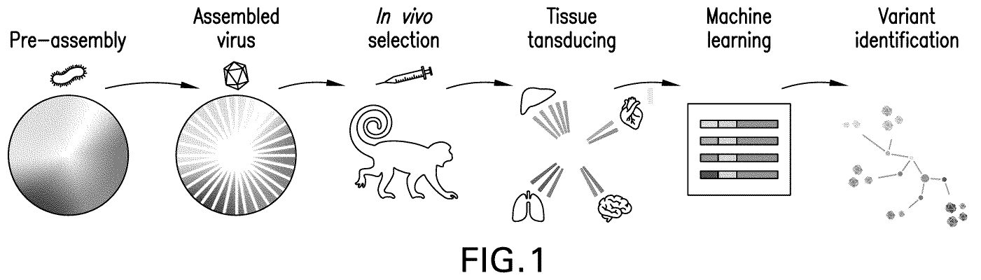

Disclosed herein is a system for high throughput engineering of engineered AAV capsids with modified function, including increased or decreased infectivity of desired tissues, such as increased or decreased liver tropism, or increased targeting of the central nervous system (CNS). A general schematic of the process is shown in FIG. 1, however, it should be understood that the present disclosure also encompasses reasonable variations or extensions to the method that are understood to those of ordinary skill in the art. As shown in FIG. 1, the method may begin with production of a capsid library with theoretical diversity of 5×10¹¹ unique sequence variants. Higher or lower theoretical diversities are also encompassed herein. For example, a capsid library may have a theoretical diversity of from about 1×10\hat{ }3 to about 1×10\hat{ }20. The library may then be cloned into plasmids, transformed into bacteria, and subsequently library plasmids are screened for productive virion assembly in a production cell line. The assembled virions may then be administered intravenously into non-human primates (NHP). After a period sufficient for distribution, infection, and stable transduction, the NHP may be sacrificed, organs harvested, and sequences of AAV capsids in each tissue may be determined by deep sequencing.

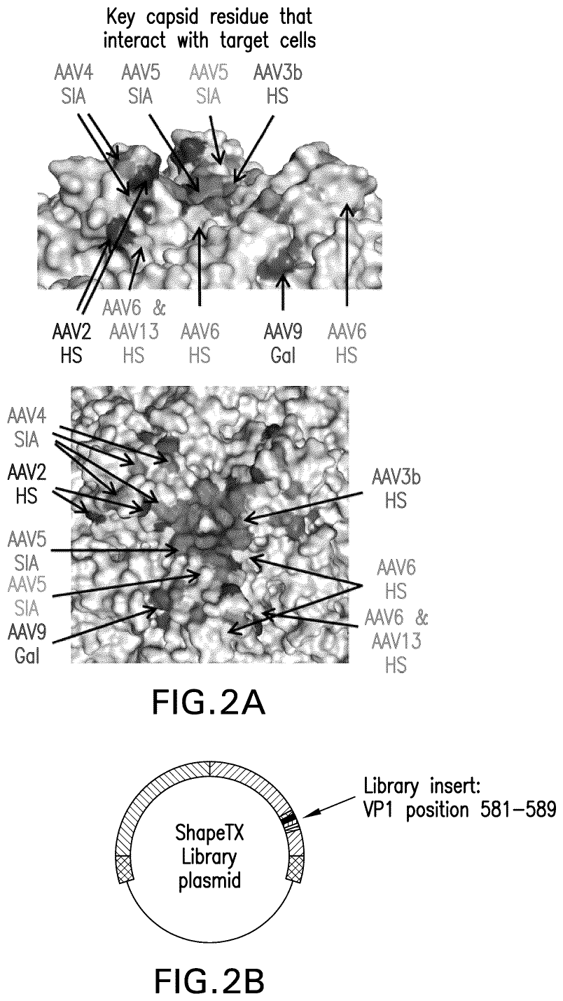

FIG. 2A provides a side view (top panel) and top view (bottom panel) of the surface of a prototype AAV virion, identifying residues of known AAV capsids—including AAV2, AAV5, AAV6, and AAV9—that have been shown in the research literature to interact with target cells. These target-interacting residues correspond to amino acids 581-589 in the AAV5 VP1 capsid protein.

FIG. 2B shows the salient elements of the library plasmid, illustrating rep and cap coding sequences positioned between AAV ITRs. In the illustrated embodiment, further described in Example 1, variation is introduced into each of residues 581-589 of the AAV5 cap protein (“Library variant region”). Each of the 20 natural amino acids is introduced at each of the 9 positions, providing a theoretical library diversity of 20⁹ (20\hat{ }9; approximately 5×10¹¹) unique sequence variants.

The area targeted for engineering is the most likely to interact with target cell receptors, and relatively tolerant to changes without disrupting virion assembly. Unlike earlier approaches that add unstructured peptides that protrude above the virion 3-fold axis of symmetry, the current approach introduces sequence diversity that alters the characteristics of the binding pocket. In addition, this approach may change the overall structure of the receptor-binding trimer, allowing for altered allosteric interactions outside the binding pocket (e.g., AAVR PKD1). Introduced diversity is non-random, thereby reducing missense and frameshifts of randomized libraries.

By cloning the polynucleotide encoding the capsid variants into the packaged viral genome (between the ITRs), the recombinant virions with variant capsids carry polynucleotides having their cognate mutation, so the unique variant providing the desired function can be identified by sequencing packaged virus or infected cells.

In some embodiments, the capsid is a capsid selected from AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 or AAVhu68 (described in WO2020/033842, incorporated herein by reference in its entirety). The hu68 capsid is described in WO 2018/160582, incorporated herein by reference in its entirety.

Such capsids may comprise a region corresponding to the 581-589 region of the AAV5 VP1, and as such analogous engineered VP capsids with desired tissue tropism, ability to assemble, and exhibit various other desired traits are encompassed herein. Thus, any one of the engineered AAV5 VP capsid polypeptides disclosed herein having a mutation in a region corresponding to the 581 to 589 region of AAV5 VP1 may be inserted into the corresponding region in any one of the other AAV capsids described herein and the present disclosure encompasses such variants.

In some embodiments, the capsid is a derivative, modification, or pseudotype of AAV1, AAV2, AAV3, AAV4, AAV5, AAV6, AAV7, AAV8, AAV9, AAV 10, AAV11, AAV 12, AAV 13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 or AAVhu68.

In some embodiments, capsid protein is a chimera of capsid proteins from two or more serotype selected from AAV1, AAV2, rAAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16 (described in WO2020/033842, incorporated herein by reference in its entirety). In certain embodiments, the capsid is an rh32.33 capsid, described in U.S. Pat. No. 8,999,678, incorporated herein by reference in its entirety.

Such capsids may comprise a region corresponding to 581-589 of the AAV5 VP1, and as such analogous engineered VP capsids with desired tissue tropism, ability to assemble, and exhibit various other desired traits are encompassed herein.

### 6.2. VP-Encoding Polynucleotides, Vectors, and Vector Libraries

Accordingly, in a first aspect, polynucleotides are provided. The polynucleotides encode an adeno-associated virus (AAV) VP1 capsid polypeptide having the amino acid sequence of SEQ ID NO:2, wherein Xaa1, Xaa2, Xaa3, Xaa4, Xaa5, Xaa6, Xaa7, Xaa8, and Xaa9 are each independently selected from the 20 naturally occurring amino acids-using standard one letter codes, from A, R, N, D, C, E, Q, G, H, I, L, K, M, F, P, S, T, W, Y, and V. The polypeptide includes at least one mutation of the native AAV5 capsid and thus does not have the sequence of SEQ ID NO: 1. In addition, the polypeptide does not have the sequence of SEQ ID NO: 3, SEQ ID NO: 4, SEQ ID NO: 5, SEQ ID NO: 6, SEQ ID NO: 7, or SEQ ID NO: 8.

In some embodiments, the polynucleotide encodes an AAV VP1 capsid polypeptide that further comprises one or more mutations at an amino acid residue outside of the 581-589 region, with reference to SEQ ID NO: 1, wherein the resulting recombinant capsid is capable of forming an assembled virion that exhibits desired tissue targeting.

In another aspect, a vector capable of replication in prokaryotic cells is provided, wherein the vector comprises the polynucleotide described immediately above. In typical embodiments, the vector is a plasmid encoding a replication-competent AAV genome.

In a further aspect, a library is provided. The library comprises a plurality of vectors comprising the AAV capsid-encoding polynucleotides. In some embodiments, the vectors are plasmids, and the plurality of plasmids comprise a plurality of different AAV VP-encoding polynucleotides.

In various library embodiments, at least one of Xaa1, Xaa2, Xaa3, Xaa4, Xaa5, Xaa6, Xaa7, Xaa8, and Xaa9 of SEQ ID NO: 2 is invariant. Such invariant residues are also referred to herein as “framework” residues. Framework residues may contribute to competence of the capsid to assemble into functional virions or infect a particular target cell or tissue

In some library embodiments, one residue of Xaa1, Xaa2, Xaa3, Xaa4, Xaa5, Xaa6, Xaa7, Xaa8, and Xaa9 of SEQ ID NO: 2 is invariant. In particular embodiments, the invariant residue is the native amino acid of AAV5 VP1 at that position within the VP1 primary amino acid sequence. In particular embodiments, the invariant residue is an amino acid other than the native amino acid of AAV5 VP1 at that position. In some embodiments, two of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, three of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, four of Xaa1, Xaa2, Xaa3, Xaa4, Xaa5, Xaa6, Xaa7, Xaa8, and Xaa9 or SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, five of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, six of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions.

In particular embodiments the rAAV VP1 capsid at position 587 (Xaa7) is not A, C, D, E, F, G, H, I, K, M, P, Q, R, V, W, or Y. In some embodiments, position 587 can be N, S, or T. In particular embodiments, the rAAV VP1 capsid at position 582 (Xaa2) is not G, V, L, or I.

In various embodiments, the library encodes at least 1×10⁹ different AAV VP capsid polypeptides, at least 2.5×10⁹ different AAV VP capsid polypeptides, at least 5×10⁹ different AAV VP capsid polypeptides, at least 7.5×10⁹ different AAV VP capsid polypeptides, at least 1×10¹⁰ different AAV VP capsid polypeptides, at least 2.5×10¹⁰ different AAV VP capsid polypeptides, at least 5×10¹⁰ different AAV VP capsid polypeptides, at least 7.5×10¹⁰ different AAV VP capsid polypeptides, at least 1×10¹¹ different AAV VP capsid polypeptides, at least 2.5×10¹¹ different AAV VP capsid polypeptides, or at least 5×10¹¹ different AAV VP capsid polypeptides.

In another aspect, prokaryotic cells comprising the vectors are provided. In some embodiments, the prokaryotic cell is an E. coli cell and the vector is a plasmid.

In a related aspect, libraries are provided, the library comprising a plurality of E. coli cells, wherein the plurality of cells comprise a plurality of plasmids, wherein the plurality of plasmids comprise a plurality of different AAV VP-encoding polynucleotides.

In various library embodiments, at least one of Xaa1, Xaa2, Xaa3, Xaa4, Xaa5, Xaa6, Xaa7, Xaa8, and Xaa9 of SEQ ID NO: 2 is invariant.

In some library embodiments, one of Xaa1, Xaa2, Xaa3, Xaa4, Xaa5, Xaa6, Xaa7, Xaa8, and Xaa9 is invariant of SEQ ID NO: 2. In particular embodiments, the invariant residue is the native amino acid of AAV5 VP1 at that position within the VP1 primary amino acid sequence. In particular embodiments, the invariant residue is an amino acid other than the native amino acid of AAV5 VP1 at that position. In some embodiments, two of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, three of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, four of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, five of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, six of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions.

In some embodiments, the library encodes at least 1×10⁹ different AAV VP capsid polypeptides, at least 2.5×10⁹ different AAV VP capsid polypeptides, at least 5×10⁹ different AAV VP capsid polypeptides, at least 7.5×10⁹ different AAV VP capsid polypeptides, at least 1×10¹⁰ different AAV VP capsid polypeptides, at least 5×10¹⁰ different AAV VP capsid polypeptides, at least 7.5×10¹⁰ different AAV VP capsid polypeptides, at least 1×10¹¹ different AAV VP capsid polypeptides, at least 2.5×10¹¹ different AAV VP capsid polypeptides, or at least 5×10¹¹ different AAV VP capsid polypeptides.

### 6.3. VP Polypeptides, Peptide Libraries

In another aspect, AAV VP1 capsid polypeptides are provided. The polypeptide has the amino acid sequence of SEQ ID NO: 2, wherein Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V. The polypeptide includes at least one mutation as compared to native AAV VP1, and thus does not have the sequence of SEQ ID NO: 1. In addition, the polypeptide does not have the sequence of SEQ ID NO: 3, SEQ ID NO: 4, SEQ ID NO: 5, SEQ ID NO: 6, SEQ ID NO: 7, or SEQ ID NO: 8.

In some embodiments, the polypeptide further comprises one or more mutations at an amino acid residue outside of the 581-589 region, with reference to SEQ ID NO: 1, wherein the resulting recombinant capsid is capable of forming an assembled virion that exhibits desired tissue targeting.

In a further aspect, libraries are provided, the libraries comprising a plurality of polypeptides as described immediately above, the plurality having different primary amino acid sequences.

In various library embodiments, at least one of Xaa1, Xaa2, Xaa3, Xaa4, Xaa5, Xaa6, Xaa7, Xaa8, and Xaa9 of SEQ ID NO: 2 is invariant.

In some library embodiments, one of Xaa1, Xaa2, Xaa3, Xaa4, Xaa5, Xaa6, Xaa7, Xaa8, and Xaa9 of SEQ ID NO: 2 is invariant. Such invariant residues are also referred to herein as “framework” residues. In particular embodiments, the invariant residue is the native amino acid of AAV5 VP1 at that position within the VP1 primary amino acid sequence. In particular embodiments, the invariant residue is an amino acid other than the native amino acid of AAV5 VP1 at that position. In some embodiments, two of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, three of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, four of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, five of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, six of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions.

In some embodiments, library comprises at least 1×10⁹ different AAV VP capsid polypeptides, at least 2.5×10⁹ different AAV VP capsid polypeptides, at least 5×10⁹ different AAV VP capsid polypeptides, at least 7.5×10⁹ different AAV VP capsid polypeptides, at least 1×10¹⁰ different AAV VP capsid polypeptides, at least 2.5×10¹⁰ different AAV VP capsid polypeptides, at least 5×10¹⁰ different AAV VP capsid polypeptides, at least 7.5×10¹⁰ different AAV VP capsid polypeptides, at least 1×10¹¹ different AAV VP capsid polypeptides, at least 2.5×10¹¹ different AAV VP capsid polypeptides, or at least 5×10¹¹ different AAV VP capsid polypeptides.

In certain embodiments, the library comprises at least from about 1×10⁵ to at least about 5×10¹¹ different AAV VP capsid polypeptides. In certain embodiments, the library comprises at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, at least about 1×10⁷, at least about 2×10⁷, at least about 3×10⁷, at least about 4×10⁷, at least about 5×10⁷, at least about 6×10⁷, at least about 7×10⁷, at least about 8×10⁷, at least about 9×10⁷, at least about 1×10⁸, at least about 2×10⁸, at least about 3×10⁸, at least about 4×10⁸, at least about 5×10⁸, at least about 6×10⁸, at least about 7×10⁸, at least about 8×10⁸, at least about 9×10⁸, at least about 1×10⁹, at least about 2×10⁹, at least about 3×10⁹, at least about 4×10⁹, at least about 5×10⁹, at least about 6×10⁹, at least about 7×10⁹, at least about 8×10⁹, at least about 9×10⁹, at least about 1×10¹⁰, at least about 2×10¹⁰, at least about 3×10¹⁰, at least about 4×10¹⁰, at least about 5×10¹⁰, at least about 6×10¹⁰, at least about 7×10¹⁰, at least about 8×10¹⁰, at least about 9×10¹⁰, at least about 1×10¹¹, at least about 2×10¹¹, at least about 3×10¹¹, at least about 4×10¹¹, or at least about 5×10¹¹ AAV VP capsid polypeptides.

In certain embodiments, provided herein is a recombinant adeno-associated virus AAV VP1 capsid polypeptide having at least one mutation in a residue of region 581 to residue 589 in SEQ ID NO: 1, inclusive, wherein the mutation confers at least about a two-fold increased accumulation of an AAV virion having said AAV VP1 capsid polypeptide in a non-liver tissue as compared to a liver tissue, as compared to wildtype AAV virion having a wildtype AAV5 VP1 capsid polypeptide, and wherein the AAV VP1 capsid polypeptide does not have the sequence of any of SEQ ID NO: 1, SEQ ID NO:3, SEQ ID NO:4, SEQ ID NO:5, SEQ ID NO:6, SEQ ID NO:7, and SEQ ID NO:8.

6.4. rAAV Virions, Virion Libraries

In another aspect, recombinant AAV virions (rAAV) are provided. The virion comprises an AAV VP capsid polypeptide as described above.

In some embodiments, the rAAV has increased tropism for primate and human liver as compared to a rAAV having the native AAV5 VP1 capsid polypeptide (SEQ ID NO:1). In some embodiments, the rAAV has increased ability to assemble, or exhibits greater virion stability, as compared to a rAAV having the native AAV5 VP1 capsid polypeptide (SEQ ID NO:1).

In some embodiments, the rAAV has reduced tropism for human liver as compared to a rAAV having the native AAV5 VP1 capsid polypeptide (SEQ ID NO:1).

In some embodiments, the rAAV has increased ability to cross the blood-brain barrier following intravenous administration as compared to a rAAV having the native AAV5 VP1 capsid polypeptide (SEQ ID NO:1).

In certain of these embodiments, the rAAV has increased ability to infect one or more brain regions selected from hippocampus, dentate gyrus, cerebral cortex, temporal cortex, occipital cortex, thalamus, forebrain, substantia nigra, hypothalamus, and cerebellum following intravenous, intrathecal, intracerebral ventricular, or intracisternal magna administration, as compared to a rAAV having the native AAV5 VP1 capsid polypeptide (SEQ ID NO:1).

In some embodiments, the rAAV has increased ability to infect one or more brain regions selected from hippocampus, dentate gyrus, cerebral cortex, temporal cortex, occipital cortex, thalamus, forebrain, substantia nigra, hypothalamus, and cerebellum following intravenous, intrathecal, intracerebral ventricular, or intracisternal magna administration and also has reduced tropism for all non CNS tissues, including being detargeted for cardiac tissue, as compared to a rAAV having the native AAV5 VP1 capsid polypeptide (SEQ ID NO:1).

In some embodiments, the rAAV has increased ability to infect human retinal cells following intravitreal injection as compared to a rAAV having the native AAV5 VP1 capsid polypeptide (SEQ ID NO:1).

In some embodiments, the rAAV has increased ability to infect human skeletal muscle following intravenous administration as compared to a rAAV having a VP1 capsid polypeptide having the native AAV5 VP1 capsid polypeptide (SEQ ID NO:1).

In some embodiments, the rAAV has increased ability to infect a tissue selected from adipose, adrenal gland, aorta, brain (including hippocampus: dentate gyrus, CA1 and CA3; cerebellum, caudate, putamen, midbrain, pons, hypothalamus, cortex-including occipital, temporal and forebrain; substantia nigra, and thalamus), bone marrow, cecum, colon, dorsal root ganglion, duodenum, epididymis, esophagus, eye, gallbladder, heart, ileum, jejunum, kidney, lung, lymph nodes, mammary gland, ovary, pancreas, parathyroid gland, peripheral nerve, pituitary, prostate, salivary gland, seminal vesicle, skeletal muscle, skin, spinal cord, spleen, stomach, testis, thymus, thyroid, trachea, urinary bladder, uterus, and vagina following intravenous administration, as compared to a rAAV having the native AAV5 capsid polypeptide (SEQ ID NO:1).

Additionally, provided are polynucleotide sequences encoding the rAAV capsid VP proteins described herein.

In a further aspect, libraries are provided that comprise a plurality of rAAV as described above. The plurality of rAAV comprise a plurality of VP capsid polypeptides having different primary amino acid sequences.

In various library embodiments, at least one of Xaa1, Xaa2, Xaa3, Xaa4, Xaa5, Xaa6, Xaa7, Xaa8, and Xaa9 of SEQ ID NO: 2 is invariant.

In some library embodiments, one of Xaa1, Xaa2, Xaa3, Xaa4, Xaa5, Xaa6, Xaa7, Xaa8, and Xaa9 of SEQ ID NO: 2 is invariant. Such invariant residues are also referred to herein as “framework” residues. In particular embodiments, the invariant residue is the native amino acid of AAV5 VP1 at that position within the VP1 primary amino acid sequence. In particular embodiments, the invariant residue is an amino acid other than the native amino acid of AAV5 VP1 at that position. In some embodiments, two of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, three of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, four of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, five of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions. In some embodiments, six of Xaa1, Xaa2, Xaa3, Xaa4, Xaa5, Xaa6, Xaa7, Xaa8, and Xaa9 of SEQ ID NO: 2 are invariant. In particular embodiments, each invariant residue is the native amino acid of AAV5 VP1 at the respective positions. In particular embodiments, each invariant residue is an amino acid other than the native amino acid of AAV5 VP1 at the respective positions.

In some embodiments, the library comprises at least 1×10⁹ different AAV VP capsid polypeptides, at least 2.5×10⁹ different AAV VP capsid polypeptides, at least 5×10⁹ different AAV VP capsid polypeptides, at least 7.5×10⁹ different AAV VP capsid polypeptides, at least 1×10¹⁰ different AAV VP capsid polypeptides, at least 2.5×10¹⁰ different AAV VP capsid polypeptides, at least 5×10¹⁰ different AAV VP capsid polypeptides, at least 7.5×10¹⁰ different AAV VP capsid polypeptides, at least 1×10¹¹ different AAV VP capsid polypeptides, at least 2.5×10¹¹ different AAV VP capsid polypeptides, or at least 5×10¹¹ different AAV VP capsid polypeptides.

### 6.5. Pharmaceutical Compositions

In another aspect, pharmaceutical compositions are provided. The pharmaceutical composition comprises a rAAV as described above and a pharmaceutically acceptable carrier.

A pharmaceutical composition can comprise a first active ingredient. The first active ingredient can comprise a viral vector as described herein and/or any payload as described herein. The pharmaceutical composition can be formulated in unit dose form. The pharmaceutical composition can comprise a pharmaceutically acceptable excipient, diluent, or carrier. The pharmaceutical composition can comprise a second, third, or fourth active ingredient—such as to facilitate enhanced gene replacement, RNA editing, DNA editing, or imaging.

A pharmaceutical composition described herein can compromise an excipient. An excipient can comprise a cryo-preservative, such as DMSO, glycerol, polyvinylpyrrolidone (PVP), or any combination thereof. An excipient can comprise a cryo-preservative, such as a sucrose, a trehalose, a starch, a salt of any of these, a derivative of any of these, or any combination thereof. An excipient can comprise a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. An excipient can comprise a surfactant, a sugar, an amino acid, an antioxidant, a salt, a non-ionic surfactant, a solubilizer, a triglyceride, an alcohol, or any combination thereof. An excipient can comprise sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerol, xanthan rubber, soy isoflavones, polysorbate 80, ethyl alcohol, water, teprenone, or any combination thereof.

Compositions and methods provided herein can utilize pharmaceutical compositions. The compositions described throughout can be formulated into a pharmaceutical and be used to treat a human or mammal, in need thereof, diagnosed with a disease. In some cases, pharmaceutical compositions can be used prophylactically.

The compositions provided herein can be utilized in methods provided herein. Any of the provided compositions provided herein can be utilized in methods provided herein. In some cases, a method comprises at least partially preventing, reducing, ameliorating, and/or treating a disease or condition, or a symptom of a disease or condition. A subject can be a human or non-human. A subject can be a mammal (e.g., rat, mouse, cow, dog, pig, sheep, horse). A subject can be a vertebrate or an invertebrate. A subject can be a laboratory animal. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician).

### 6.6. Methods of Treatment or Detection

In some aspects, the present disclosure provides for methods of treatment using an rAAV virion having any one of the engineered AAV VP capsid polypeptide sequences disclosed herein. In some aspects, the present disclosure provides for methods of detection using an rAAV virion having any one of the engineered AAV VP capsid polypeptide sequences disclosed herein. The method comprises administering an effective amount of the pharmaceutical composition comprising rAAV virions having any one of the AAV VP capsid polypeptide sequences disclosed herein to a subject in need thereof. The rAAV virions encapsidate any payload, including those payloads disclosed herein.

In some embodiments, the effective amount is at least 1×10⁸ viral genomes per dose. In some embodiments, the effective amount is at least 5×10⁸ viral genomes/dose, 7.5×10⁸ viral genomes/dose, at least 1×10⁹ viral genomes/dose, at least 2.5×10⁹ viral genomes/dose, at least 5×10⁹ viral genomes/dose.

In some embodiments, the effective amount is at least 1×10¹¹ viral genomes/kg patient weight, at least 5×10¹¹ viral genomes/kg, at least 1×10¹² viral genomes/kg, at least 5×10¹² viral genomes/kg, at least 1×10¹³ viral genomes/kg, at least 1×10¹⁴ viral genomes/kg, or at least 5×10¹⁴.

In some embodiments, the rAAV virion is administered via a systemic administration route including enteral routes of administration and parenteral routes of administration. The rAAV virion may be administered intravenously. In some embodiments, the rAAV may be administered intramuscularly. In some embodiments, the rAAV may be administered intraperitoneally. In some embodiments, the rAAV may be administered topically. In some embodiments, the rAAV may be administered orally. In particular embodiments, the rAAV virion is administered intravenously. In some embodiments, the rAAV is administered intrathecally. In some embodiments, the rAAV is administered by intracerebral ventricular injection. In some embodiments, the rAAV is administered by intracisternal magna administration. In some embodiments, the rAAV is administered by intravitreal injection.

In various embodiments, the patient suffers from one of the conditions listed in TABLE 1, below. In particular embodiments, the patient suffers from one of the conditions listed in TABLE 1 and the rAAV includes the transgene product associated therewith in TABLE 1.

In some embodiments, an rAAV virion of the present disclosure, having any of the engineered AAV VP capsid polypeptide sequences disclosed herein, comprises a vector genome, the vector genome comprising a therapeutic polynucleotide or payload. In further embodiments, said payload may be under control of regulatory sequences that direct expression in infected human cells. In some embodiments, the payload comprises a therapeutic polynucleotide encoding any genetically encodable payload, such as an RNA (e.g., a guide RNA), a suppressor tRNA, a transgene, or a genome modifying entity.

In some embodiments, the therapeutic polynucleotide encodes a guide RNA, a tRNA, a suppressor tRNA, a siRNA, a miRNA, an mRNA, a shRNA, a circular RNA, or an antisense oligonucleotide (ASO), a ribozyme, a DNAzyme, an aptamer, or any combination thereof. In some embodiments, the therapeutic polynucleotide encodes a linear therapeutic polynucleotide or a circular therapeutic polynucleotide.

In some embodiments, the therapeutic polynucleotide encodes a therapeutic protein (a transgene). In particular embodiments, the transgene encodes a protein selected from the targets suitable for modification or transgene products of TABLE 1.

TABLE 1
Suitable Therapeutic Targets

| Primary | | |
|---|---|---|
| gene | | |
| delivery | | Target of a Therapeutic |
| target | Condition | Polynucleotide |
| Brain/ | AADC deficiency | AADC |
| CNS | Alzheimer's Disease | Multiple, including |
| | | APP, SNCA, MAPT, ApoE, |
| | | NGF, TERT |
| | Tauopathies | MAPT |
| | Synucleinopathies | SNCA |
| | Batten disease (CLN2) | CLN2 |
| | Batten disease (CLN3) | CLN3 |
| | Batten disease (CLN6) | CLN6 |
| | MPS-IIIB | NAGLU |
| | Frontotemporal dementia | GRN |
| | with GRN mutations (FTD- | |
| | GRN) | |
| | Parkinson's Disease with | GBA1 |
| | GBA1 mutations (PD- | |
| | GBA) and neuronpathic | |
| | Gaucher's disease | |
| | Synucleinopathies | GBA1 + alpha-synuclein |
| | Gaucher disease type 2 | GBA |
| | Canavan Disease | ASPA |
| | Parkinson disease | AADC |
| | Parkinson disease | GDNF |
| | Parkinson disease | Neurturin |
| | Parkinson disease | GAD |
| | Parkinson disease | NTN |
| | Parkinson disease | hFOXG1 |
| | Parkinson disease | hKCNQ2 |
| | Parkinson disease | hFMR1 |
| | Parkinson disease | anti-Tau/miRNA |
| | Parkinson disease | EPM2A or EPM2B |
| | Parkinson disease | LRRK2 |
| | Parkinson's Disease | LRRK2 |
| | Parkinson's Disease | SNCA |
| | Tay-Sachs Disease | HEXA |
| | Huntington's disease | IT15 |
| | Huntington's disease | CYP46A1 |
| | Huntington's disease | HTT |
| | Protocki-Lupski Syndrome | IT15 |
| | Amyotrophic lateral | C9orf72 |
| | sclerosis | |
| | Amyotrophic lateral | SOD1 |
| | sclerosis | |
| | Down syndrome | DYRK1A |
| | Sanfilippo disease type A | SGSH |
| | Sanfilippo disease type B | hNAGLU |
| | (Nervous system) | HEXB and HEXA |
| | (Nervous system) | human codon-optimized |
| | | CLN1 complementary |
| | | DNA |
| | (Nervous system) | SURF1 |
| | (Nervous system) | anti-UBE3A-ATS shRNA |
| | (Nervous system) | hSLC6A1 |
| | Rett syndrome | MECP2 |
| Spinal | spinal muscular atrophy | SMN |
| cord | (SMA) | |
| | Giant axonal neuropathy | GAN |
| | Chronic Pain | Nav1.7 |
| | spinocerebellar ataxias | ATXN3 |
| | (SCAs), | |
| Eye | Achromatopsia | CNGB3 |
| | Choroideraemia | REP1 |
| | ad Retinitis Pigmentosa | NRL, RDH12, PRPH2 (RDS), |
| | | RHO, RPGR, SNRNP200, |
| | | NR2E3, IMPDH1, CRX, |
| | | HK1, IMPDH2, SNRNP200 |
| | Stargardt disease | ABCA4 |
| | Usher Syndrome 2A | USH2A |
| | Wet AMD, Dry AMD | NRP1 |
| | Leber congenital amaurosis | RPE65 |
| | (LCA) | |
| | Leber hereditary optic | ND4 |
| | neuropathy (LHON) | |
| | retinitis pigmentosa (RP, | RLBP1 |
| | including RLBP1) | |
| | Wet AMD | Anti-VEGF antibody |
| | X-linked retinitis | RPGR |
| | pigmentosa (X-linked RP) | |
| | X-linked retinoschisis | RS1 |
| Liver | Crigler-Najjar syndrome | UGT1A1 |
| | Familial | LDLR |
| | Hypercholesterolemia (FH | |
| | homozygous) | |
| | Glycogen storage disease | G6PC |
| | type 1A (GSD1a) | |
| | Haemophilia A | FVIII |
| | Haemophilia B | FIX |
| | Mucopolysaccharidosis I | ZFN1, ZFN2 and |
| | (MPS-I) | IDUA donor |
| | Mucopolysaccharidosis II | ZFN1, ZFN2 and |
| | (MPS-II) | IDS donor |
| | Liver Mucopolysaccharidosis | SGSH |
| | IIIA (MPS-IIIA) | |
| | Mucopolysaccharidosis | NAGLU |
| | IIIB (MPS-IIIB) | |
| | Mucopolysaccharidosis VI | ARSB |
| | (MPS-VI) | |
| | hydroxylase deficiency | CYP21A2 |
| | Cardiovascular disease | PCSK9 |
| | Porphyria and Acute | ALAS1 |
| | hepatic porphyria | |
| | Hemochromatosis | HFE |
| | Cholesteryl ester storage | LIPA |
| | disease | |
| | Wilson disease | ATP7B |
| | Adult polyglucosan body | GBE1 (also muscle cells) |
| | disease | |
| | hepatic steatosis | hSLC13A5 |
| | Alpha-1 antitrypsin | SERPINA1 |
| | deficiency | |
| | Ornithine | OTC |
| | Transcarbamylase | |
| | Deficiency (OTC | |
| | deficiency) | |
| | Alpha-1 antitrypsin | A1AT |
| | deficiency (A1AT | |
| | deficiency) | |
| Muscle | Charcot-Marie-Tooth | NTF3 |
| | disease type 1A (CMT1A) | |
| | Duchenne muscular | Micro-dystrophin |
| | dystrophy (DMD) | |
| | Duchenne muscular | Mini-dystrophin |
| | dystrophy (DMD) | |
| | Dysferlinopathy | DYSF |
| | Pompe disease | GAA |
| | Limb-girdle muscular | FKRP |
| | dystrophies (LGMD) | |
| | Muscle (2i/R9) | |
| | Duchenne muscular | DMD |
| | dystrophy (DMD) | |
| | Facioscapulohumeral | DUX4 |
| | Dystrophy | |
| | Myotonic Dystrophy | DMPK |
| | Glycogen storage disorders | anti-GYS1 miRNA |
| | X-linked myotubular | MTM1 |
| | myopathy (X-linked MTM) | |
| | euchromatic histone-lysine | anti-EHMT2 shRNA |
| | N-methyltransferase 2 | |
| Other | (Associated with hearing | TMC1 |
| | loss) | |
| | Other Obesity (adipose tissue) | CIDEC, SCD1, GNB3 |
| | Bone (osteoclasts) | CLCN7 |
| | Chondrocytes | FGFR3 |
| | Primary Hyperoxaluria | HAO1 |
| | Type 1 (kidney) | |
| | Primary Hyperoxaluria | LDHA |
| | (kidney) | |
| | Acromegaly (multi-organ) | GHR |
| | Asthma (WBCs; | Mex3B |
| | neutrophils, eosinophils) | |
| | Alport syndrome (kidney) | COL4A5 |
| | Transthyretin amyloidosis | TTR |
| | (familial) (multi-organ) | |
| | Charcot-Marie Tooth | PMP22 |
| | Syndrome (PNS/Sciatic | |
| | Nerve; Schwann Cells) | |
| | Angelman syndrome | UBE3A |
| | (nervous system) | |
| | Congestive heart failure | I-1c |
| | (heart) | |
| | Methylmalonic acidemia | MMUT, MMAA, MMAB, |
| | (MMA) (Kidneys) | MMADHC, MCEE |
| | Cystic fibrosis (lung) | CFTR |
| | HIV infections | PG9 antibody |
| | HIV infections | VRC07 antibody |
| Anemia- | Hemophilia | F8 (Factor VIII), |
| related | | F9 (Factor IX) |
| disorders | | |
| Sickle-cell | sickle cell anemia | HBB |
| related | sickle cell hemoglobin C | Hemoglobin |
| disorders | disease | |
| | sickle cell thalassemia | beta thalassemia |
| | disease | |

In some embodiments, the therapeutic polynucleotide encodes a therapeutic RNA. In some embodiments the therapeutic polynucleotide encodes an RNA, such as a guide RNA (including an engineered or synthetic guide RNA) for genome editing or for RNA editing.

In some embodiments, the therapeutic polynucleotide encodes a tRNA or a modified tRNA (engineered or synthetic tRNA). For example, the tRNA or modified tRNA can be a suppressor tRNA. The suppressor tRNA can be engineered to have an anticodon region that recognizes a stop codon, such as any premature stop codon (opal, ochre, or amber stop codons).

In some embodiments, the therapeutic polynucleotide (e.g., a therapeutic RNA, a tRNA, or a genome modifying entity) can target a gene listed in TABLE 1 or any gene associated with a neurologic disease, Parkinson's disease, Alzheimer's disease, a Tauopathy, Stargardt disease, alpha-1 antitrypsin deficiency, Duchenne's muscular dystrophy, Rett syndrome, cystic fibrosis, or any genetic disease. In some embodiments, the targeted gene may be ABCA4, AAT, SERPINA1, SERPINA1 E342K, HEXA, LRRK2, SNCA, DMD, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof. In some embodiments, the therapeutic polynucleotide is a gene therapy payload (e.g., a transgene) and, thus, may itself be one of the genes listed in TABLE 1 or any gene associated with a neurologic disease, Parkinson's disease, Alzheimer's disease, a Tauopathy, Stargardt disease, alpha-1 antitrypsin deficiency, Duchenne's muscular dystrophy, Rett syndrome, cystic fibrosis, or any genetic disease. In some embodiments, the transgene may be ABCA4, AAT, SERPINA1, SERPINA1 E342K, HEXA, LRRK2, SNCA, DMD, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof.

In some embodiments, the therapeutic polynucleotide encodes genome modifying entities. For example, a genome modifying entity may be a DNA editing enzyme, an RNA editing enzyme, a transcriptional activator, or a transcriptional repressor. The DNA editing enzyme may be any DNA editing enzyme, including any CRISPR/Cas systems, meganucleases, zinc-finger nucleases, (ZFNs), TALE Nucleases (TALENs and megaTALENS). The CRISPR/Cas system can be a Cas3, Cas8, Cas10, Cas9, Cas4, Cas12, or Cas13. The RNA editing enzyme may be ADAR. In some embodiments, the ADAR is a human ADAR1 or human ADAR2. The transcriptional activator may be VP64. A transcriptional repressor may be KRAB. Such genome modifying entities may target any gene listed in TABLE 1 for editing.

In some embodiments, the present disclosure provides for rAAV virions having an engineered AAV VP capsid polypeptide, where the virion encapsidates any one of or any combination of the therapeutic payloads disclosed herein. In some embodiments, multiple copies of the therapeutic payload are encapsidated.

In some embodiments, the therapeutic polynucleotide is a polynucleotide capable of serving as a homology template for homology-directed repair.

In some embodiments, an rAAV virion of the present disclosure, having any of the engineered AAV VP capsid polypeptide sequences disclosed herein, comprises a vector genome, the vector genome comprising a detectable polynucleotide or payload. In further embodiments, said payload may be under control of regulatory sequences that direct expression in infected human cells. Examples of detectable polynucleotides include, but are not limited to, any genetically encodable detectable moiety. For example, a genetically encodable detectable moiety may be a fluorescent protein such as EGFP, GFP, YFP, RFP, CFP, or any variants thereof. In some embodiments, the present disclosure provides for rAAV virions having an engineered AAV VP capsid polypeptide, where the virion encapsidates any one of or any combination of the detectable payloads disclosed herein. In some embodiments, multiple copies of the detectable payload are encapsidated.

In some embodiments, the present disclosure provides for rAAV virions having an engineered AAV VP capsid polypeptide, where the virion encapsidates any one of or any combination of the therapeutic payloads and detectable payloads disclosed herein. For example, an rAAV of the present disclosure having an engineered AAV VP capsid polypeptide may encapsidate a transgene and a fluorescent protein. As another example, an rAAV of the present disclosure having an engineered AAV VP capsid polypeptide may encapsidate a therapeutic RNA (e.g., a guide RNA) and a fluorescent protein.

### 6.7. In Vivo Selected VP Polypeptides

In a further aspect, engineered (synonymously, recombinant) adeno-associated virus (AAV) VP capsid polypeptides identified using the methods described herein are provided.

In some embodiments, the engineered adeno-associated virus (AAV) viral protein (VP) capsid polypeptide has an amino acid sequence at least 70% identical to SEQ ID NO: 1, wherein the engineered AAV VP capsid polypeptide has at least one substitution as compared to SEQ ID NO: 1 in the region from residue 581 to residue 589 of SEQ ID NO: 1, inclusive, wherein the capsid polypeptide is capable of assembling into a recombinant AAV virion (rAAV), and wherein the VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8.

In some embodiments, the engineered adeno-associated virus (AAV) viral protein (VP) capsid polypeptide has an amino acid sequence at least 70% identical to SEQ ID NO: 1, wherein the engineered AAV VP capsid polypeptide has at least one substitution as compared to SEQ ID NO: 1 in the region from residue 581 to residue 589 of SEQ ID NO: 1, inclusive, wherein the capsid polypeptide is capable of assembling into a recombinant AAV virion (rAAV), wherein the at least one substitution confers higher tropism for a central nervous system (CNS) tissue on the rAAV as compared to an rAAV virion having an AAV5 VP capsid polypeptide of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8.

In particular embodiments, the engineered adeno-associated virus (AAV) viral protein (VP) capsid polypeptide has an amino acid sequence at least 75%, 80%, 85%, 90%, 91%, 92%, 93%, 94%, 95%, 96%, 97%, 98% identical to the sequence of SEQ ID NO: 1.

In some embodiments, the AAV VP capsid polypeptide has an amino acid sequence of SEQ ID NO: 2, wherein Xaa1, Xaa2, Xaa3, Xaa4, Xaa5, Xaa6, Xaa7, Xaa8, and Xaa9 are each independently selected from any amino acid, wherein the capsid polypeptide is capable of assembling into a recombinant AAV virion (rAAV), and wherein the VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8, optionally with further mutations elsewhere in the VP capsid polypeptide

In some embodiments, the AAV VP capsid polypeptide has an amino acid sequence of SEQ ID NO: 2, wherein Xaa1, Xaa2, Xaa3, Xaa4, Xaa5, Xaa6, Xaa7, Xaa8, and Xaa9 are each independently selected from any amino acid, wherein the capsid polypeptide is capable of assembling into a recombinant AAV virion (rAAV), wherein the at least one substitution confers higher tropism for a central nervous system (CNS) tissue on the rAAV as compared to an rAAV virion having an AAV5 VP capsid polypeptide of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8, optionally with further mutations elsewhere in the VP capsid polypeptide

In some embodiments, the engineered adeno-associated virus (AAV) viral protein (VP) capsid polypeptide has an amino acid sequence of SEQ ID NO: 2, wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V; wherein the engineered AAV VP capsid polypeptide is capable of assembling into a recombinant AAV virion (rAAV); and wherein the rAAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8.

In some embodiments, the region of the engineered VP capsid polypeptide from residue 581 to residue 589, inclusive, has a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO:7118-SEQ ID NO:10,117. In particular embodiments, the region of the engineered VP capsid polypeptide from residue 581 to residue 589, inclusive, has a sequence that is identical to any one of SEQ ID NO:7118-SEQ ID NO:10,117.

In some embodiments, the engineered adeno-associated virus (AAV) viral protein (VP) capsid polypeptide is an engineered AAV5 viral capsid protein, wherein the engineered AAV VP5 capsid polypeptide has at least one substitution as compared to SEQ ID NO: 1 in the region from residue 581 to residue 589 of SEQ ID NO: 1, inclusive; wherein the capsid polypeptide is capable of assembling into a recombinant AAV virion (rAAV); wherein the at least one substitution confers higher tropism for a central nervous system (CNS) tissue on the rAAV as compared to an rAAV virion having an AAV5 VP capsid polypeptide of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8, optionally with further mutations elsewhere in the VP protein.

In some embodiments, the AAV VP capsid polypeptides have an amino acid sequence of SEQ ID NO: 2, wherein Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V; and wherein the polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8.

In some embodiments, the engineered AAV VP capsid polypeptide comprises a polypeptide sequence represented by the formula: (A)-(X)-(B)
wherein:
(A) is the polypeptide sequence of SEQ ID NO: 47438 (VAYNVGGQMATNNQSSTTAP residues 561-580 of SEQ ID NO: 2);
(X) is the polypeptide sequence comprising amino acid residues Xaa1, Xaa2, Xaa3, Xaa4, Xaa5, Xaa6, Xaa7, Xaa8, and Xaa9 of SEQ ID NO: 2; and
(B) is the polypeptide sequence of SEQ ID NO:47439 (IVPGSVWMERDVYLQGPIWA residues 590-609 of SEQ ID NO: 2;
wherein Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V; and
wherein the capsid polypeptide is capable of assembling into a recombinant AAV virion (rAAV); and;
wherein the polypeptide does not have the sequence of any of SEQ ID NO:1, SEQ ID NO: 3, SEQ ID NO:4, SEQ ID NO:5, SEQ ID NO:6, SEQ ID NO:7, and SEQ ID NO:8.

In some embodiments, the engineered AAV VP capsid polypeptide comprises a polypeptide sequence represented by the formula: (A)-(X)-(B) wherein:
(A) is the polypeptide sequence of SEQ ID NO: 47438 (residues 561-580 of SEQ ID NO: 2 VAYNVGGQMATNNQSSTTAP);
(X) is a polypeptide sequence selected from the list of polypeptides in Table 8 (SEQ ID NOs: 115-1114) or Table 10 (SEQ ID NOs: 7118-8117) that confers CNS tissue tropism on a recombinant AAV virion (rAAV); and
(B) is the polypeptide sequence of SEQ ID NO: 47439 (residues 590-609 of SEQ ID NO: 2: (IVPGSVWMERDVYLQGPIWA)); and wherein the capsid polypeptide is capable of assembling into the rAAV and,
the capsid does not have the sequence of any of SEQ ID NO:1, SEQ ID NO:3, SEQ ID NO:4, SEQ ID NO:5, SEQ ID NO:6, SEQ ID NO:7, and SEQ ID NO:8.

In some embodiments, the engineered AAV VP capsid polypeptide confers CNS tissue tropism, wherein the CNS tissue is selected from the group consisting of hippocampus: (dentate gyrus, CA1 and CA3); cerebellum, hypothalamus, cortex: (occipital, temporal and forebrain); substantia nigra, thalamus, and any combination thereof.

In some embodiments, the engineered AAV VP capsid polypeptide comprises a polypeptide sequence represented by the formula: (A)-(X)-(B) wherein:
(A) is the polypeptide sequence of SEQ ID NO: 47438 (residues 561-580 of SEQ ID NO: 2: (VAYNVGGQMATNNQSSTTAP));
(X) is a polypeptide sequence selected from the polypeptides of SEQ ID NO: 115-1114 or SEQ ID NO: 1118-47437 that confer corresponding tissue tropism on a recombinant AAV virion (rAAV); and
(B) is the polypeptide sequence of SEQ ID NO: 47439 (residues 590-609 of SEQ ID NO: 2: (IVPGSVWMERDVYLQGPIWA)); and wherein the capsid polypeptide is capable of assembling into the rAAV and,
the capsid does not have the sequence of any of SEQ ID NO: 1, SEQ ID NO:3, SEQ ID NO:4, SEQ ID NO:5, SEQ ID NO:6, SEQ ID NO:7, and SEQ ID NO:8.

Described below are engineered mutated AAV5 VP1 polypeptide sequences that confer stable or improved virion assembly, tissue tropism, or both. In some embodiments, the present disclosure provides an AAV5 VP1 capsid polypeptide having a sequence homology of no more than 98.7% to SEQ ID NO: 1, wherein the AAV5 capsid polypeptide sequence has at least one mutation in a region from a position corresponding to 581 to a position corresponding to 589 of SEQ ID NO: 1.

Also encompassed herein are rAAVs composed of engineered AAV5 VP2 capsid polypeptides and engineered AAV5 VP3 capsid polypeptides having the sequences disclosed in the Tables of the Examples (e.g., Table 7, 10, 12, 14, 16, 18, 20, 22, 24, 26, 28, 30, 32, 34, 36, 39, 41, 43, 45, 47, 49, 51, 53, 55, 57, 59, 61, 63, 65, 67, 69, 71, 72, 73, 74, 75, 76, 77, 78, 79, 80, 81, 82, 83, 84, 85, and 86) at the regions in AAV5 VP2 (amin acid residues 445 to 453) and AAV5 VP3 (amino acid residues 389-397) corresponding to the amino acids in the AAV5 VP1 581 to 589 region.

6.7.1. In Vivo Selected VP Polypeptides that Confer Increased Liver Tropism

In various embodiments, the present disclosure provides a mutated VP polypeptide capable of forming an assembled virion that exhibits increased tropism for liver tissue when compared to a virion that comprises the AAV5 VP1 capsid polypeptide of SEQ ID NO: 1. In this section of the disclosure, liver tissue tropism is determined by the frequency of a given amino acid residue occurring at a specified position corresponding to position 581 to position 589 of SEQ ID NO: 1 (generalized in SEQ ID NO: 2) over the frequency of that given amino acid residue in the total library of virus administered to NHP.

In some embodiments, Xaa1 is selected from A, G, K, M, N, Q, R, S, or T.

In some embodiments, Xaa1 is selected from A, K, M, or T.

In some embodiments, Xaa1 is K.

In additional embodiments, Xaa2 is selected from A, C, H, I, K, S, T, or V.

In some embodiments, Xaa2 is selected from A, S, T, or V.

In some embodiments, Xaa2 is T.

In additional embodiments, Xaa3 is selected from A, G, H, K, M, N, Q, R, S, T, or V.

In some embodiments, Xaa3 is selected from A, M, or T.

In some embodiments, Xaa3 is A or T.

In additional embodiments, Xaa4 is selected from L, M, P, Q, R, T, or W.

In some embodiments, Xaa4 is selected from L, P, Q, or T.

In some embodiments, Xaa4 is P.

In additional embodiments, Xaa5 is selected from F, H, I, K, M, T, or Y.

In some embodiments, Xaa5 is selected from H, I, or Y.

In some embodiments, Xaa5 is Y.

In additional embodiments, Xaa6 is selected from E, G, H, L, M, N, Q, T, or W.

In some embodiments, Xaa6 is selected from N, or Q.

In some embodiments, Xaa6 is N.

In additional embodiments, Xaa7 is selected from A, C, G, H, L, M, R or S.

In some embodiments, Xaa7 is selected from A, C, H or M.

In some embodiments, Xaa7 is A.

In additional embodiments, Xaa8 is selected from A, C, D, F, G, H, M, Q, S, V, W, or Y.

In some embodiments, Xaa8 is selected from G, M, Q, or S.

In some embodiments, Xaa8 is G.

In additional embodiments, Xaa9 is selected from A, C, E, G, H, M, N, P, Q, S, V, or W.

In some embodiments, Xaa9 is selected from E, G, or P.

In some embodiments, Xaa9 is G.

In particular embodiments, the sequence of Xaa1-Xaa9 of the engineered (recombinant) capsid polypeptide is selected from the amino acid sequence provided in TABLE 2.

In some embodiments, the engineered AAV capsid and corresponding virion exhibits increased liver tropism, when compared with AAV5 wildtype capsid and corresponding virion. This increased tropism can range from about 1.5, 2.0, 2.5, 3.0, 3.5, 4.0, 4.5, 5.0, 5.5, 6.0, 6.5, 7.0, 7.5, 8.0, 8.5, 9.0, 9.5, to about 10.0-fold when compared to a virion that comprises the AAV5 VP1 capsid polypeptide of SEQ ID NO: 1.

TABLE 2
Sequences of the 581 to 589 Region in
AAV5 VP1 Capsid Polypeptide that Drive
Liver Tropism

| Var # | Translation |
|---|---|
| SEQ ID NO: 10 | IAVASHAHG |
| SEQ ID NO: 11 | SAYPKSEDV |
| SEQ ID NO: 12 | SSMQCAMRP |
| SEQ ID NO: 13 | ITVVVGEVN |
| SEQ ID NO: 14 | SEAGDRCSD |
| SEQ ID NO: 15 | ECACGHVHL |
| SEQ ID NO: 16 | TFTNAGKMC |
| SEQ ID NO: 17 | LASPYLIDA |
| SEQ ID NO: 18 | TTQLYEVGS |
| SEQ ID NO: 19 | TVHPIDHAT |
| SEQ ID NO: 20 | TWMMVHRYV |
| SEQ ID NO: 21 | VWYRCDVRH |
| SEQ ID NO: 22 | HWADKALDW |
| SEQ ID NO: 23 | MHTMKNCGW |
| SEQ ID NO: 24 | LCNRMQADQ |
| SEQ ID NO: 25 | WKATPGGQC |
| SEQ ID NO: 26 | ARHTFPYQT |
| SEQ ID NO: 27 | MHMSYHWRE |
| SEQ ID NO: 28 | MPKAEIFVP |
| SEQ ID NO: 29 | DKTICQHQL |
| SEQ ID NO: 30 | TTRGSERCS |
| SEQ ID NO: 31 | HTPLAWHGD |
| SEQ ID NO: 32 | SCTPFQQHG |
| SEQ ID NO: 33 | AHQLAMVMN |
| SEQ ID NO: 34 | AAAQRSRYV |
| SEQ ID NO: 35 | DCMTSMLGP |
| SEQ ID NO: 36 | RYLFAVWRY |
| SEQ ID NO: 37 | MMNMQMDRM |
| SEQ ID NO: 38 | AVQWIQWVG |
| SEQ ID NO: 39 | QSQPHVQYN |
| SEQ ID NO: 40 | THTFAYHNN |
| SEQ ID NO: 41 | GWQDEQMHL |
| SEQ ID NO: 42 | TSKQETPQQ |
| SEQ ID NO: 43 | CSNSPACLC |
| SEQ ID NO: 44 | VDAHTETNG |
| SEQ ID NO: 45 | CNEKGYHWH |
| SEQ ID NO: 46 | IADIGRMQW |
| SEQ ID NO: 47 | TEWPYGVAF |
| SEQ ID NO: 48 | QCQYQTAWW |
| SEQ ID NO: 49 | GPSGIFHCG |
| SEQ ID NO: 50 | MAHAIDALQ |
| SEQ ID NO: 51 | MHHCYQTFA |
| SEQ ID NO: 52 | GQTVFHYDG |
| SEQ ID NO: 53 | CNAIIAMPC |
| SEQ ID NO: 54 | GTITLMPTQ |
| SEQ ID NO: 55 | TVQCEMDVC |
| SEQ ID NO: 56 | CQHNTPFVQ |
| SEQ ID NO: 57 | RWTDTNFRG |
| SEQ ID NO: 58 | KYEQKWMMD |
| SEQ ID NO: 59 | MLYSTTCWK |
| SEQ ID NO: 60 | ASWVGSFAQ |
| SEQ ID NO: 61 | TTKQYHTME |
| SEQ ID NO: 62 | PGGCYRMME |
| SEQ ID NO: 63 | RNAVCLYRH |
| SEQ ID NO: 64 | VIHEMLSAC |
| SEQ ID NO: 65 | YGEQKYNHS |
| SEQ ID NO: 66 | ANQWENAVK |
| SEQ ID NO: 67 | TSAWWWCSP |
| SEQ ID NO: 68 | EQTMMDLVY |
| SEQ ID NO: 69 | YAMPRNPNV |
| SEQ ID NO: 70 | MHKVWWMKN |
| SEQ ID NO: 71 | AILQMYTAQ |
| SEQ ID NO: 72 | CSTPANSCP |
| SEQ ID NO: 73 | KVVEAELWQ |
| SEQ ID NO: 74 | IANIDPSRV |
| SEQ ID NO: 75 | QLTTTKPLR |
| SEQ ID NO: 76 | ACWRFECDD |
| SEQ ID NO: 77 | QINPEHGGC |
| SEQ ID NO: 78 | MAPPFHVYE |
| SEQ ID NO: 79 | DNWGTWTWT |
| SEQ ID NO: 80 | DESSCVAHG |
| SEQ ID NO: 81 | VAATFNNKV |
| SEQ ID NO: 82 | HVVPEPWAF |
| SEQ ID NO: 83 | TQMPALAEW |
| SEQ ID NO: 84 | NVTNQQYDH |
| SEQ ID NO: 85 | NMTEYVAYR |
| SEQ ID NO: 86 | NVSTVHQPL |
| SEQ ID NO: 87 | QTGPMLHSW |
| SEQ ID NO: 88 | ECMHKHASY |
| SEQ ID NO: 89 | LQAHVVRCT |
| SEQ ID NO: 90 | VGDRYSSMG |
| SEQ ID NO: 91 | PQGLIPMWA |
| SEQ ID NO: 92 | MYVHKGYRS |
| SEQ ID NO: 93 | AVPQYQKAE |
| SEQ ID NO: 94 | ERMMILCSP |
| SEQ ID NO: 95 | NFGFTCPVY |
| SEQ ID NO: 96 | SQIWNVAAY |
| SEQ ID NO: 97 | MWGQQGTWA |
| SEQ ID NO: 98 | QAMMMTMMN |
| SEQ ID NO: 99 | AHTANEFSP |
| SEQ ID NO: 100 | DAHYVYEKG |
| SEQ ID NO: 101 | CNNWIWAHE |
| SEQ ID NO: 102 | NHNLMWVVS |
| SEQ ID NO: 103 | ATMWGDCDY |
| SEQ ID NO: 104 | EWMQEFAGP |
| SEQ ID NO: 105 | QDGSVEWAF |
| SEQ ID NO: 106 | WCPQPPGGN |
| SEQ ID NO: 107 | AECQIWYDW |
| SEQ ID NO: 108 | NAVKFVCED |
| SEQ ID NO: 109 | TQCFASCVA |
| SEQ ID NO: 110 | TVNNHDIGY |

6.7.2. In Vivo Selected Engineered VP Polypeptides that are Competent for Assembly into rAAV

In various preferred embodiments, the mutated (engineered, recombinant) VP capsid polypeptides of the present disclosure are capable of forming an assembled virion, and in some instances that exhibit similar or improved stability when compared to a virion that comprises the AAV5 VP1 capsid polypeptide of SEQ ID NO: 1.

The frequency of a given amino acid residue occurring in assembled, purified viruses at a specified position corresponding to position 581 to position 589 of SEQ ID NO: 1 (generalized in SEQ ID NO: 2) over the frequency of that given amino acid residue occurring at the specified position in the entire plasmid library was analyzed to identify sequence rules for capsids that preferentially virally assembly.

Disclosed herein are engineered AAV5 VP capsid polypeptides capable of forming an assembled virion that may exhibit similar or improved stability as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein Xaa1 is selected from A, D, E, G, L, M, N, Q, S, T, or V, or Xaa1 is selected from A, D, E, M, or T. In some embodiments, Xaa1 is E; or Xaa2 is selected from A, C, D, E, G, H, I, N, P, Q, S, T, or V, or Xaa2 is selected from A, S, T, or V, or Xaa2 is A; or wherein Xaa3 is selected from A, D, E, G, H, M, N, Q, S, T, or V, or Xaa3 is selected from D, E, N, Q or T, or Xaa3 is D or T; or wherein Xaa4 is selected from A, D, E, G, H, N, P, Q, S, or T, or Xaa4 is selected from D, E, P, or Q, or Xaa4 is E; or wherein Xaa5 is selected from A, C, D, E, G, H, N, Q, S, T, or Y, or Xaa5 is selected from D, E, N, Q or T, or Xaa5 is N; or wherein Xaa6 is selected from A, D, E, G, H, N, P, Q, S, or T, or Xaa6 is selected from D, N, or Q, or Xaa6 is D; or wherein Xaa7 is selected from A, C, D, E, G, H, N, Q, S, or T, or Xaa7 is selected from A, D, E or G, or Xaa7 is A; or wherein Xaa8 is selected from A, C, D, E, G, H, N, Q, S, or T, or Xaa8 comprises A, D, G, or S, or Xaa8 is G; or wherein Xaa9 is selected from A, D, E, G, H, N, P, Q, S, or T, or Xaa9 is selected from A, D, G, or P, or Xaa9 is G.

In various embodiments, the VP polypeptide is capable of forming an assembled virion, and in some instances exhibits similar or improved stability when compared to a virion that comprises the AAV5 VP1 capsid polypeptide of SEQ ID NO:1.

In some embodiments, Xaa1 is selected from A, D, E, G, L, M, N, Q, S, T, or V.

In some embodiments, Xaa1 is selected from A, D, E, M, or T. In some embodiments, Xaa1 is E.

In some embodiments, Xaa2 is selected from A, C, D, E, G, H, I, N, P, Q, S, T, or V. In some embodiments, Xaa2 is selected from A, S, T, or V. In some embodiments, Xaa2 is A.

In some embodiments, Xaa3 is selected from A, D, E, G, H, M, N, Q, S, T, or V. In some embodiments, Xaa3 is selected from D, E, N, Q or T. In some embodiments, Xaa3 is D or T.

In some embodiments, Xaa4 is selected from A, D, E, G, H, N, P, Q, S, or T. In some embodiments, Xaa4 is selected from D, E, P, or Q. In some embodiments, Xaa4 is E.

In some embodiments, Xaa5 is selected from A, C, D, E, G, H, N, Q, S, T, or Y. In some embodiments, Xaa5 is selected from D, E, N, Q or T. In some embodiments, Xaa5 is N.

In some embodiments, Xaa6 is selected from A, D, E, G, H, N, P, Q, S, or T. In some embodiments, Xaa6 is selected from D, N, or Q. In some embodiments, Xaa6 is D.

In some embodiments, Xaa7 is selected from A, C, D, E, G, H, N, Q, S, or T. In some embodiments, Xaa7 is selected from A, D, E or G. In some embodiments, Xaa7 is A.

In some embodiments, Xaa8 is selected from A, C, D, E, G, H, N, Q, S, or T. In some embodiments, Xaa8 comprises A, D, G, or S. In some embodiments, Xaa8 is G.

In some embodiments, Xaa9 is selected from A, D, E, G, H, N, P, Q, S, or T. In some embodiments, Xaa9 is selected from A, D, G, or P. In some embodiments, Xaa9 is G.

6.7.3. In Vivo Selected Mutated VP Polypeptides that are Competent for Assembly into rAAV Virions and Exhibit Decreased Liver Tropism

The present disclosure provides AAV5 virions with a VP capsid polypeptide having at least one mutation in a region with residues that interact with target cells, where the at least one mutation confers decreased liver tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives decreased liver tropism.

The frequency of a given amino acid residue occurring at a specified position corresponding to position 581 to position 589 of SEQ ID NO: 1 (generalized in SEQ ID NO: 2) in variants not identified in liver over the frequency of that given amino acid residue occurring at the specified position in variants forming assembled virus was analyzed to identify a set of sequence rules for capsids that preferentially detarget liver tissue.

Disclosed herein are engineered AAV5 VP capsid polypeptides capable of forming an assembled virion that exhibits decreased liver tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein Xaa1 is not K, or Xaa1 is not A, K, M, or T, or Xaa1 is not A, G, K, M, N, Q, R, S, or T; or wherein Xaa2 is not T, or Xaa2 is not A, S, T, or V, or Xaa2 is not A, C, H, I, K, S, T, or V; or wherein Xaa3 is not A or T, or Xaa3 is not A, M, or T, or Xaa3 is not A, G, H, K, M, N, Q, R, S, T, or V; or wherein Xaa4 is not P, or wherein Xaa4 is not L, P, Q, or T, or Xaa4 is not L, M, P, Q, R, T, or W; or wherein Xaa5 is not Y, or Xaa5 is not H, I, or Y, or Xaa5 is not F, H, I, K, M, T, or Y; or wherein Xaa6 is not N, or Xaa6 is not N, or Q, or Xaa6 is not E, G, H, L, M, N, Q, T, or W; or wherein Xaa7 is not A, or Xaa7 is not A, C, H or M, or Xaa7 is not A, C, G, H, L, M, R or S; or wherein Xaa8 is not G, or Xaa8 is not G, M, Q, or S, or Xaa8 is not A, C, D, F, G, H, M, Q, S, V, W, or Y; or wherein Xaa9 is not G, or Xaa9 is not E, G, or P, or Xaa9 is not A, C, E, G, H, M, N, P, Q, S, V, or W.

In certain embodiments, Xaa1 is not K, and wherein the VP capsid is capable of forming an assembled virion that exhibits decreased tropism for liver tissue when compared to a virion that comprises the AAV5 VP1 capsid of SEQ ID NO: 1.

In certain embodiments, Xaa1 is not A, K, M, or T, and wherein the VP capsid is capable of forming an assembled virion that exhibits decreased tropism for liver tissue when compared to a virion that comprises the AAV5 VP1 capsid of SEQ ID NO:1.

In certain embodiments, Xaa1 is not A, G, K, M, N, Q, R, S, or T, and wherein the VP capsid is capable of forming an assembled virion that exhibits decreased tropism for liver tissue when compared to a virion that comprises the AAV5 VP1 capsid of SEQ ID NO:1.

In certain embodiments, Xaa2 is not T, and wherein the VP capsid is capable of forming an assembled virion that exhibits decreased tropism for liver tissue when compared to a virion that comprises the AAV5 VP1 capsid of SEQ ID NO:1.

In certain embodiments, Xaa2 is not A, S, T, or V, and wherein the VP capsid is capable of forming an assembled virion that exhibits decreased tropism for liver tissue when compared to a virion that comprises the AAV5 VP1 capsid of SEQ ID NO:1.

In certain embodiments, Xaa2 is not A, C, H, I, K, S, T, or V, and wherein the VP capsid is capable of forming an assembled virion that exhibits decreased tropism for liver tissue when compared to a virion that comprises the AAV5 VP1 capsid of SEQ ID NO:1.

In certain embodiments, Xaa3 is not A or T, and wherein the VP capsid is capable of forming an assembled virion that exhibits decreased tropism for liver tissue when compared to a virion that comprises the AAV5 VP1 capsid of SEQ ID NO:1.

In certain embodiments, Xaa3 is not A, M, or T, and wherein the VP capsid is capable of forming an assembled virion that exhibits decreased tropism for liver tissue when compared to a virion that comprises the AAV5 VP1 capsid of SEQ ID NO:1.

In certain embodiments, Xaa3 is not A, G, H, K, M, N, Q, R, S, T, or V, and wherein the VP capsid is capable of forming an assembled virion that exhibits decreased tropism for liver tissue when compared to a virion that comprises the AAV5 VP1 capsid of SEQ ID NO:1.

In certain embodiments, Xaa4 is not P, and wherein the VP capsid is capable of forming an assembled virion that exhibits decreased tropism for liver tissue when compared to a virion that comprises the AAV5 VP1 capsid of SEQ ID NO:1.

In certain embodiments, wherein Xaa4 is not L, P, Q, or T, and wherein the VP capsid is capable of forming an assembled virion that exhibits decreased tropism for liver tissue when compared to a virion that comprises the AAV5 VP1 capsid of SEQ ID NO:1.

In certain embodiments, Xaa4 is not L, M, P, Q, R, T, or W, and wherein the VP capsid is capable of forming an assembled virion that exhibits decreased tropism for liver tissue when compared to a virion that comprises the AAV5 VP1 capsid of SEQ ID NO:1.

In certain embodiments, Xaa5 is not Y, and wherein the VP capsid is capable of forming an assembled virion that exhibits decreased tropism for liver tissue when compared to a virion that comprises the AAV5 VP1 capsid of SEQ ID NO:1.

In certain embodiments, Xaa5 is not H, I, or Y, and wherein the VP capsid is capable of forming an assembled virion that exhibits decreased tropism for liver tissue when compared to a virion that comprises the AAV5 VP1 capsid of SEQ ID NO:1.

In certain embodiments, Xaa5 is not F, H, I, K, M, T, or Y, and wherein the VP capsid is capable of forming an assembled virion that exhibits decreased tropism for liver tissue when compared to a virion that comprises the AAV5 VP1 capsid of SEQ ID NO:1.

In certain embodiments, Xaa6 is not N, and wherein the VP capsid is capable of forming an assembled virion that exhibits decreased tropism for liver tissue when compared to a virion that comprises the AAV5 VP1 capsid of SEQ ID NO:1.

In certain embodiments, Xaa6 is not N, or Q, and wherein the VP capsid is capable of forming an assembled virion that exhibits decreased tropism for liver tissue when compared to a virion that comprises the AAV5 VP1 capsid of SEQ ID NO:1.

In certain embodiments, Xaa6 is not E, G, H, L, M, N, Q, T, or W, and wherein the VP capsid is capable of forming an assembled virion that exhibits decreased tropism for liver tissue when compared to a virion that comprises the AAV5 VP1 capsid of SEQ ID NO:1.

In certain embodiments, Xaa7 is not A, and wherein the VP capsid is capable of forming an assembled virion that exhibits decreased tropism for liver tissue when compared to a virion that comprises the AAV5 VP1 capsid of SEQ ID NO:1.

In certain embodiments, Xaa7 is not A, C, H or M, and wherein the VP capsid is capable of forming an assembled virion that exhibits decreased tropism for liver tissue when compared to a virion that comprises the AAV5 VP1 capsid of SEQ ID NO:1.

In certain embodiments, Xaa7 is not A, C, G, H, L, M, R or S, and wherein the VP capsid is capable of forming an assembled virion that exhibits decreased tropism for liver tissue when compared to a virion that comprises the AAV5 VP1 capsid of SEQ ID NO:1.

In certain embodiments, Xaa8 is not G, and wherein the VP capsid is capable of forming an assembled virion that exhibits decreased tropism for liver tissue when compared to a virion that comprises the AAV5 VP1 capsid of SEQ ID NO:1.

In certain embodiments, Xaa8 is not G, M, Q, or S, and wherein the VP capsid is capable of forming an assembled virion that exhibits decreased tropism for liver tissue when compared to a virion that comprises the AAV5 VP1 capsid of SEQ ID NO:1.

In certain embodiments, Xaa8 is not A, C, D, F, G, H, M, Q, S, V, W, or Y, and wherein the VP capsid is capable of forming an assembled virion that exhibits decreased tropism for liver tissue when compared to a virion that comprises the AAV5 VP1 capsid of SEQ ID NO: 1.

In certain embodiments, Xaa9 is not G, and wherein the VP capsid is capable of forming an assembled virion that exhibits decreased tropism for liver tissue when compared to a virion that comprises the AAV5 VP1 capsid of SEQ ID NO:1.

In certain embodiments, Xaa9 is not E, G, or P, and wherein the VP capsid is capable of forming an assembled virion that exhibits decreased tropism for liver tissue when compared to a virion that comprises the AAV5 VP1 capsid of SEQ ID NO:1.

In certain embodiments, Xaa9 is not A, C, E, G, H, M, N, P, Q, S, V, or W, and wherein the VP capsid is capable of forming an assembled virion that exhibits decreased tropism for liver tissue when compared to a virion that comprises the AAV5 VP1 capsid of SEQ ID NO: 1.

The present disclosure encompasses variant VP capsids that have increased tissue tropism, compared to the AAV5 VP1 capsid of SEQ ID NO:1, for any of the following tissues: adipose, adrenal gland, aorta, brain (including hippocampus: dentate gyrus, CA1 and CA3; cerebellum, caudate, putamen, midbrain, pons, hypothalamus, cortex-including occipital, temporal and forebrain; substantia nigra, and thalamus), bone marrow, cecum, colon, dorsal root ganglion, duodenum, epididymis, esophagus, eye, gallbladder, heart, ileum, jejunum, kidney, lung, lymph nodes, mammary gland, ovary, pancreas, parathyroid gland, peripheral nerve, pituitary, prostate, salivary gland, seminal vesicle, skeletal muscle, skin, spinal cord, spleen, stomach, testis, thymus, thyroid, trachea, urinary bladder, uterus, and vagina.

6.7.4. In Vivo Selected Mutated VP Polypeptides that Detarget Liver Tissue

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in non-liver over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), skeletal muscle, heart, lung, spleen, lymph node, bone marrow, mammary gland, skin, adrenal gland, thyroid, colon, sciatic nerve, and spinal cord tissues, liver) was analyzed to identify a set of sequence rules for capsids that preferentially detarget liver tissue. Identification of positional frequency rules from in vivo data is described in detail in EXAMPLE 4. With reference to TABLE 6B in EXAMPLE 4, and SEQ ID NO: 2 (AAV5 VP1), the following amino acids can, thus, be independently mutated, in any combination, at any one or more positions Xaa1-Xaa9, to provide a VP1 capsid with reduced liver tropism as compared wildtype AAV5 VP1 capsid (SEQ ID NO: 1), where liver tropism here refers to properties that are deterministic for liver transduction over properties that are deterministic for transduction of all other harvested tissues.

Disclosed herein are engineered AAV5 VP capsid polypeptides capable of forming an assembled virion that exhibits decreased liver tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein Xaa1 excludes K; or Xaa1 excludes A, K, M, or T; or Xaa1 excludes A, G, K, M, N, Q, R, S, or T; or Xaa2 excludes T; or Xaa2 excludes A, S, T, or V; or Xaa2 excludes A, C, H, I, K, S, T, or V; or Xaa3 excludes A or T; or Xaa3 excludes A, M, or T; or Xaa3 excludes A, G, H, K, M, N, Q, R, S, T, or V; or Xaa4 excludes P; or Xaa4 excludes L, P, Q, or T; or Xaa4 excludes L, M, P, Q, R, T, or W; or Xaa5 excludes Y; or Xaa5 excludes H, I, or Y; or Xaa5 excludes F, H, I, K, M, T, or Y; or Xaa6 excludes N; or Xaa6 excludes N, or Q; or Xaa6 excludes E, G, H, L, M, N, Q, T, or W; or Xaa7 excludes A; or Xaa7 excludes A, C, H or M; or Xaa7 excludes A, C, G, H, L, M, R or S; or Xaa8 excludes G; or Xaa8 excludes G, M, Q, or S; or Xaa8 excludes A, C, D, F, G, H, M, Q, S, V, W, or Y; or Xaa9 excludes G; or Xaa9 excludes E, G, or P; or Xaa9 excludes A, C, E, G, H, M, N, P, Q, S, V, or W.

Disclosed herein are engineered AAV5 VP capsid polypeptides capable of forming an assembled virion that exhibits decreased liver tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 excludes K. In some embodiments, Xaa1 excludes A, K, M, or T. In some embodiments, Xaa1 excludes A, G, K, M, N, Q, R, S, or T. In some embodiments, Xaa2 excludes T. In some embodiments, Xaa2 excludes A, S, T, or V. In some embodiments, Xaa2 excludes A, C, H, I, K, S, T, or V. In some embodiments, Xaa3 excludes A or T. In some embodiments, Xaa3 excludes A, M, or T. In some embodiments, Xaa3 excludes A, G, H, K, M, N, Q, R, S, T, or V. In some embodiments, Xaa4 excludes P. In some embodiments, Xaa4 excludes L, P, Q, or T. In some embodiments, Xaa4 excludes L, M, P, Q, R, T, or W. In some embodiments, Xaa5 excludes Y. In some embodiments, Xaa5 excludes H, I, or Y. In some embodiments, Xaa5 excludes F, H, I, K, M, T, or Y. In some embodiments, Xaa6 excludes N. In some embodiments, Xaa6 excludes N, or Q. In some embodiments, Xaa6 excludes E, G, H, L, M, N, Q, T, or W. In some embodiments, Xaa7 excludes A. In some embodiments, Xaa7 excludes A, C, H or M. In some embodiments, Xaa7 excludes A, C, G, H, L, M, R or S. In some embodiments, Xaa8 excludes G. In some embodiments, Xaa8 excludes G, M, Q, or S. In some embodiments, Xaa8 excludes A, C, D, F, G, H, M, Q, S, V, W, or Y. In some embodiments, Xaa9 excludes G. In some embodiments, Xaa9 excludes E, G, or P. In some embodiments, Xaa9 excludes A, C, E, G, H, M, N, P, Q, S, V, or W.

Disclosed herein are engineered AAV5 VP capsid polypeptides capable of forming an assembled virion that exhibits decreased liver tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 excludes A, K, M, or T, Xaa2 excludes, Xaa3 excludes A or T, Xaa4 excludes P, Xaa5 excludes Y, Xaa6 excludes N, Xaa7 excludes A, Xaa8 excludes G, and Xaa9 excludes G.

Disclosed herein are engineered AAV5 VP capsid polypeptides capable of forming an assembled virion that exhibits decreased liver tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 excludes A, K, M, or T, or Xaa2 excludes, or Xaa3 excludes A or T, or Xaa4 excludes P, or Xaa5 excludes Y, or Xaa6 excludes N, or Xaa7 excludes A, or Xaa8 excludes G, or Xaa9 excludes G, or any combination thereof.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 21. Disclosed herein are engineered AAV5 VP capsid polypeptides capable of forming an assembled virion that exhibits decreased liver tissue tropism as compared to an rAAV virion having a wildtype AAV5 VP capsid polypeptide, wherein the engineered AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein Xaa1 is selected from an amino acid of low solubility at position Xaa1 (e.g., Xaa1 is selected from D, or P); or wherein Xaa1 is selected from an amino acid of low mutability at position Xaa1 (e.g., Xaa1 is selected from C, K, or L); or wherein Xaa2 is selected from an amino acid of low solubility at position Xaa2 (e.g., Xaa2 is selected from N, K, P, E, or D); or wherein Xaa2 is selected from an amino acid of low hydropathy at position Xaa2 (e.g., Xaa2 is selected from D, E, R, K, H, N, or Q); or wherein Xaa2 is selected from an amino acid of low charge at position Xaa2 (e.g., Xaa2 is selected from D or E); or wherein Xaa2 is selected from an amino acid of high number of total potential hydrogen bonds at position Xaa2 (e.g., Xaa2 is selected from H, N, Q, D, E, or R); or wherein Xaa2 is selected from an amino acid of medium volume at position Xaa2 (e.g., Xaa2 is selected from D, E, V, P, N, or T); or wherein Xaa3 is selected from an amino acid of low solubility at position Xaa3 (e.g., Xaa3 is selected from P or D); or wherein Xaa4 is selected from an amino acid of medium volume at position Xaa4 (e.g., Xaa4 is selected from D, E, V, P, N, or T); or wherein Xaa5 is selected from an amino acid of low solubility at position Xaa5 (e.g., Xaa5 is selected from N, P, E, or D); or wherein Xaa8 is selected from an amino acid of low solubility at position Xaa8 (e.g., Xaa8 is selected from K or Q); or wherein Xaa8 is selected from an amino acid of low hydropathy at position Xaa8 (e.g., Xaa8 is selected from K or R); or wherein Xaa8 is selected from an amino acid of high surface accessibility at position Xaa8 (e.g., Xaa8 is selected from E, R, or K); or any combination thereof.

In some embodiments, Xaa1 is selected from an amino acid of low solubility at position Xaa1. In some embodiments, Xaa1 is selected from D or P. In some embodiments, Xaa1 is selected from an amino acid of low mutability at position Xaa1. In some embodiments, Xaa1 is selected from C, K, or L. In some embodiments, Xaa2 is selected from an amino acid of low solubility at position Xaa2. In some embodiments, Xaa2 is selected from N, K, P, E, or D. In some embodiments, Xaa2 is selected from an amino acid of low hydropathy at position Xaa2. In some embodiments, Xaa2 is selected from D, E, R, K, H, N, or Q. In some embodiments, Xaa2 is selected from an amino acid of low charge at position Xaa2. In some embodiments, Xaa2 is selected from D, E. In some embodiments, Xaa2 is selected from an amino acid of high number of total potential hydrogen bonds at position Xaa2. In some embodiments, Xaa2 is selected from H, N, Q, D, E, or R. In some embodiments, Xaa2 is selected from an amino acid of medium volume at position Xaa2. In some embodiments, Xaa2 is selected from D, E, V, P, N, or T. In some embodiments, Xaa3 is selected from an amino acid of low solubility at position Xaa3. In some embodiments, Xaa3 is selected from P or D. In some embodiments, Xaa4 is selected from an amino acid of medium volume at position Xaa4. In some embodiments, Xaa4 is selected from D, E, V, P, N, or T. In some embodiments, Xaa5 is selected from an amino acid of low solubility at position Xaa5. In some embodiments, Xaa5 is selected from N, P, E, or D. In some embodiments, Xaa8 is selected from an amino acid of low solubility at position Xaa8. In some embodiments, Xaa8 is selected from K or Q. In some embodiments, Xaa8 is selected from an amino acid of low hydropathy at position Xaa8. In some embodiments, Xaa8 is selected from K or R. In some embodiments, Xaa8 is selected from an amino acid of high surface accessibility at position Xaa8. In some embodiments, Xaa8 is selected from E, R, or K.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 46438-SEQ ID NO: 47437, wherein said at least one mutation drives liver detargeting tissue tropism.

6.7.5. In Vivo Selected Mutated VP Polypeptides that Confer Increased Liver Tropism

The present disclosure provides AAV5 virions with a VP capsid polypeptide having at least one mutation in a region with residues that interact with target cells (e.g., a target liver cell in a target liver tissue of interest), where the at least one mutation confers increased liver tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased liver tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in liver over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), skeletal muscle, heart, lung, spleen, lymph node, bone marrow, mammary gland, skin, adrenal gland, thyroid, colon, sciatic nerve, and spinal cord tissues) was analyzed to identify a set of sequence rules for capsids that preferentially target liver tissue. Identification of positional frequency rules from in vivo data is described in detail in EXAMPLE 4.

Disclosed herein are engineered AAV5 VP capsid polypeptides capable of forming an assembled virion that exhibits increased liver tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein Xaa1 is selected from A, G, K, M, N, Q, R, S, or T, or

Xaa1 is selected from A, K, Q, or R, or Xaa1 is K; or wherein Xaa2 is selected from A, C, I, K, S, T, or V, or Xaa2 is selected from A, K, S, or T, or Xaa2 is A; or wherein Xaa3 is selected from A, G, I, K, M, Q, R, S, T, or V, or Xaa3 is selected from A, K, Q, S, or T, or Xaa3 is selected from K, Q, or T, or Xaa3 is K; or wherein Xaa4 is selected from A, I, K, L, P, Q, R, S, T, or V, or Xaa4 is selected from K, I, S, or V, or Xaa4 is K; or Xaa5 is selected from F, I, L, M, T, V, or Y, or wherein Xaa5 is selected from F, L, or Y, or Xaa5 is F; or Xaa6 is selected from F, H, M, N, Q, S, or Y, or wherein Xaa6 is selected from M or N, or Xaa6 is N; or Xaa7 is selected from A, C, K, M, Q or S, or wherein Xaa7 is selected from A, C, or S, or Xaa7 is S; or wherein Xaa8 is selected from A, C, F, G, M, Q, or S, or Xaa8 is selected from A, C, M, or S, or Xaa8 is C; or wherein Xaa9 is selected from E, F, L, Q, R, or Y, or Xaa9 is selected from L, Q, or R, or Xaa9 is R.

Disclosed herein are engineered AAV5 VP capsid polypeptides capable of forming an assembled virion that exhibits increased liver tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from A, G, K, M, N, Q, R, S, or T. In some embodiments, Xaa1 is selected from A, K, Q, or R. In some embodiments, Xaa1 is K. In some embodiments, Xaa2 is selected from A, C, I, K, S, T, or V. In some embodiments, is selected from A, K, S, or T, or Xaa2 is A. In some embodiments, wherein Xaa3 is selected from A, G, I, K, M, Q, R, S, T, or V, or Xaa3 is selected from A, K, Q, S, or T. In some embodiments, Xaa3 is selected from K, Q, or T. In some embodiments, Xaa3 is K. In some embodiments, Xaa4 is selected from A, I, K, L, P, Q, R, S, T, or V. In some embodiments, Xaa4 is selected from K, I, S, or V. In some embodiments, Xaa4 is K. In some embodiments, Xaa5 is selected from F, I, L, M, T, V, or Y. In some embodiments, Xaa5 is selected from F, L, or Y, or Xaa5 is F. In some embodiments, Xaa6 is selected from F, H, M, N, Q, S, or Y. In some embodiments, wherein Xaa6 is selected from M or N, or Xaa6 is N. In some embodiments, Xaa7 is selected from A, C, K, M, Q or S. In some embodiments, Xaa7 is selected from A, C, or S, or Xaa7 is S. In some embodiments, Xaa8 is selected from A, C, F, G, M, Q, or S. In some embodiments, Xaa8 is selected from A, C, M, or S, or Xaa8 is C. In some embodiments, Xaa9 is selected from E, F, L, Q, R, or Y. In some embodiments, Xaa9 is selected from L, Q, or R, or Xaa9 is R.

Disclosed herein are engineered AAV5 VP capsid polypeptides capable of forming an assembled virion that exhibits increased liver tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from A, G, K, M, N, Q, R, S, or T, Xaa2 is selected from A, C, I, K, S, T, or V, Xaa3 is selected from A, G, I, K, M, Q, R, S, T, or V, Xaa4 is selected from A, I, K, L, P, Q, R, S, T, or V, Xaa5 is selected from F, I, L, M, T, V, or Y, Xaa6 is selected from F, H, M, N, Q, S, or Y, Xaa7 is selected from A, C, or S, Xaa8 is selected from A, C, F, G, M, Q, or S, and Xaa9 is selected from E, F, L, Q, R, or Y.

Disclosed herein are engineered AAV5 VP capsid polypeptides capable of forming an assembled virion that exhibits increased liver tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from A, G, K, M, N, Q, R, S, or T, or Xaa2 is selected from A, C, I, K, S, T, or V, or Xaa3 is selected from A, G, I, K, M, Q, R, S, T, or V, or Xaa4 is selected from A, I, K, L, P, Q, R, S, T, or V, or Xaa5 is selected from F, I, L, M, T, V, or Y, or Xaa6 is selected from F, H, M, N, Q, S, or Y, or Xaa7 is selected from A, C, or S, or Xaa8 is selected from A, C, F, G, M, Q, or S, or Xaa9 is selected from E, F, L, Q, R, or Y, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 43438-SEQ ID NO: 44437, wherein said at least one mutation drives increased liver tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 20. Disclosed herein are engineered AAV5 VP capsid polypeptides capable of forming an assembled virion that exhibits increased liver tissue tropism as compared to an rAAV virion having a wildtype AAV5 VP capsid polypeptide, wherein the engineered AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein Xaa1 is selected from an amino acid of high surface accessibility (e.g., Xaa1 is selected from K, R, or E); or wherein Xaa1 is selected from an amino acid of low hydropathy (e.g., Xaa1 is selected from K, R); or wherein Xaa1 is selected from an amino acid of low amino acid mutability (e.g., Xaa1 is selected from H, P, K, or R); or wherein Xaa1 is selected from an amino acid of low amino acid solubility (e.g., Xaa1 is selected from Q, K, R); or wherein Xaa2 is selected from an amino acid of high surface accessibility (e.g., Xaa2 is selected from E, R, or K); or wherein Xaa2 is selected from an amino acid of low hydropathy (e.g., Xaa2 is selected from K, R); or wherein Xaa2 is selected from an amino acid of high amino acid volume (e.g., Xaa2 is selected from S, L, I, A, R, or K); or wherein Xaa3 is selected from an amino acid of high mutability (e.g., Xaa3 is selected from N, I, A, M, E, or D); or wherein Xaa3 is selected from an amino acid of low solubility (e.g., Xaa3 is selected from N, K, R, or E); or wherein Xaa4 is selected from an amino acid of low hydropathy (e.g., Xaa4 is selected from K or R); or wherein Xaa4 is selected from an amino acid of high amino acid volume (e.g., Xaa4 is selected from K, R, I, or L); or wherein Xaa5 is selected from an amino acid of medium amino acid solubility (e.g., Xaa5 is selected from H or T); or wherein Xaa8 is selected from an amino acid of low surface accessibility (e.g., Xaa8 is selected from V or C); or wherein Xaa8 is selected from an amino acid of low average flexibility index (e.g., Xaa8 is selected from W, V, M, A, F, L, H, or C); or any combination thereof.

In some embodiments, Xaa1 is selected from an amino acid of high surface accessibility. In some embodiments, Xaa1 is selected from K, R, or E. In some embodiments, Xaa1 is selected from an amino acid of low hydropathy. In some embodiments, Xaa1 is selected from K or R. In some embodiments, Xaa1 is selected from an amino acid of low amino acid mutability. In some embodiments, Xaa1 is selected from H, P, K, or R. In some embodiments, Xaa1 is selected from an amino acid of low amino acid solubility. In some embodiments, Xaa1 is selected from Q, K, or R. In some embodiments, Xaa2 is selected from an amino acid of high surface accessibility. In some embodiments, Xaa2 is selected from E, R, or K. In some embodiments, Xaa2 is selected from an amino acid of low hydropathy. In some embodiments, Xaa2 is selected from K or R. In some embodiments, Xaa2 is selected from an amino acid of high amino acid volume. In some embodiments, Xaa2 is selected from S, L, I, A, R, or K. In some embodiments, Xaa3 is selected from an amino acid of high mutability. In some embodiments, Xaa3 is selected from N, I, A, M, E, or D. In some embodiments, Xaa3 is selected from an amino acid of low solubility. In some embodiments, Xaa3 is selected from N, K, R, or E. In some embodiments, Xaa4 is selected from an amino acid of low hydropathy. In some embodiments, Xaa4 is selected from K, R. In some embodiments, Xaa4 is selected from an amino acid of high amino acid volume. In some embodiments, Xaa4 is selected from K, R, I, or L. In some embodiments, Xaa5 is selected from an amino acid of medium amino acid solubility. In some embodiments, Xaa5 is selected from H, T. In some embodiments, Xaa8 is selected from an amino acid of low surface accessibility. In some embodiments, Xaa8 is selected from V or C. In some embodiments, Xaa8 is selected from an amino acid of low average flexibility index. In some embodiments, Xaa8 is selected from W, V, M, A, F, L, H, or C.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 44438-SEQ ID NO: 45437, wherein said at least one mutation drives increased liver tissue tropism.

### C. Enriched Liver Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 45438-SEQ ID NO: 46437, wherein said at least one mutation drives increased liver tissue tropism.

6.7.6. In Vivo Selected Mutated VP Polypeptides that Confer Increased Central Nervous System Tropism, Positional Frequency Based Rules and ML Rules

The present disclosure provides AAV5 virions with a VP capsid polypeptide having at least one mutation in a region with residues that interact with target cells (e.g., a target CNS cell in a target CNS tissue of interest), where the at least one mutation confers increased CNS tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased central nervous system tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in central nervous system (cortex forebrain, cortex occipital, cortex temporal, thalamus, hypothalamus, substantia nigra, hippocampus DG, hippocampus CA1, hippocampus CA3, cerebellum) over the frequency of that given amino acid residue occurring at the specified position in variants identified in all other harvested tissues (liver, skeletal muscle, heart, lung, spleen, lymph node, bone marrow, mammary gland, skin, adrenal gland, thyroid, colon, sciatic nerve, and spinal cord tissues) was analyzed to identify a set of sequence rules for capsids that preferentially target central nervous system tissues. Identification of positional frequency rules from in vivo data is described in detail in EXAMPLE 5.

Disclosed herein are engineered AAV5 VP capsid polypeptides capable of forming an assembled virion that exhibits increased central nervous system tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein Xaa1 is selected from A, C, K, M, Q, R, T, or W, or Xaa1 is selected from K, Q, R, or W, or Xaa1 is K; or Xaa2 is selected from F, I, K, R, T, or W, or Xaa2 is selected from F, I, R or T, or Xaa2 is R; or Xaa3 is selected from A, H, N, R, or W, or Xaa3 is selected from A, R, or W, or Xaa3 is R; or Xaa4 is selected from E, G, I, M, Q, or R, or Xaa4 is selected from E, M, or R, or Xaa4 is R; or Xaa5 is selected from C, G, K, I, M, or R, or Xaa5 is selected from K, I, or R, or Xaa5 is I; or Xaa6 is selected from I, K, L, P, Q, R, Y, or Xaa6 is selected from K, R, or Y, or Xaa6 is R; or Xaa7 is selected from D, I, K, R, V, or W, or Xaa7 is selected from I, R, or V, or Xaa7 is V; or Xaa8 is selected from C, G, H, K, L, or V, or Xaa8 is selected from H, K, or V, or Xaa8 is H; or Xaa9 is selected from I, K, L, R, or V, or Xaa9 is selected from I, K, or R, or Xaa9 is R.

Disclosed herein are engineered AAV5 VP capsid polypeptides capable of forming an assembled virion that exhibits increased central nervous system tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from A, C, K, M, Q, R, T, or W. In some embodiments, Xaa1 is selected from K, Q, R, or W. In some embodiments, Xaa1 is K. In some embodiments, Xaa2 is selected from F, I, K, R, T, or W. In some embodiments, Xaa2 is selected from F, I, R or T. In some embodiments, Xaa2 is R. In some embodiments, Xaa3 is selected from A, H, N, R, or W. In some embodiments, Xaa3 is selected from A, R, or W. In some embodiments, Xaa3 is R. In some embodiments, Xaa4 is selected from E, G, I, M, Q, or R. In some embodiments, Xaa4 is selected from E, M, or R. In some embodiments, Xaa4 is R. In some embodiments, Xaa5 is selected from C, G, K, I, M, or R. In some embodiments, Xaa5 is selected from K, I, or R. In some embodiments, Xaa5 is I. In some embodiments, Xaa6 is selected from I, K, L, P, Q, R, Y. In some embodiments, Xaa6 is selected from K, R, or Y. In some embodiments, Xaa6 is R. In some embodiments, Xaa7 is selected from D, I, K, R, V, or W. In some embodiments, Xaa7 is selected from I, R, or V. In some embodiments, Xaa7 is V. In some embodiments, Xaa8 is selected from C, G, H, K, L, or V. In some embodiments, Xaa8 is selected from H, K, or V. In some embodiments, Xaa8 is H. In some embodiments, Xaa9 is selected from I, K, L, R, or V. In some embodiments, Xaa9 is selected from I, K, or R. In some embodiments, Xaa9 is R.

Disclosed herein are engineered AAV5 VP capsid polypeptides capable of forming an assembled virion that exhibits increased central nervous system tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from A, C, K, M, Q, R, T, or W, Xaa2 is selected from F, I, K, R, T, or W, Xaa3 is selected from A, H, N, R, or W, Xaa4 is selected from E, G, I, M, Q, or R, Xaa5 is selected from C, G, K, I, M, or R, Xaa6 is selected from I, K, L, P, Q, R, Y, Xaa7 is selected from D, I, K, R, V, or W, Xaa8 is selected from C, G, H, K, L, or V, and Xaa9 is selected from I, K, L, R, or V.

Disclosed herein are engineered AAV5 VP capsid polypeptides capable of forming an assembled virion that exhibits increased central nervous system tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from A, C, K, M, Q, R, T, or W, Xaa2 is selected from F, I, K, R, T, or W, Xaa3 is selected from A, H, N, R, or W, Xaa4 is selected from E, G, I, M, Q, or R, Xaa5 is selected from C, G, K, I, M, or R, Xaa6 is selected from I, K, L, P, Q, R, Y, Xaa7 is selected from D, I, K, R, V, or W, Xaa8 is selected from C, G, H, K, L, or V, Xaa9 is selected from I, K, L, R, or V, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 7118-SEQ ID NO: 8117, wherein said at least one mutation drives increased central nervous system tissue tropism.

### B. ML Rules

For the following set of rules described in the subsequent paragraphs in this section, favored biophysical properties and favored amino acid residues at each position in the 581 to 589 region of an engineered AAV5 VP1 capsid polypeptide, were determined using in vivo data and two ML models, which are described in EXAMPLE 19. Disclosed herein are engineered AAV5 VP capsid polypeptides capable of forming an assembled virion that exhibits increased central nervous system tissue tropism as compared to an rAAV virion having a wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein Xaa1 is selected from an amino acid of low amino acid solubility (e.g., Xaa1 is selected from K, R, or Q); or wherein Xaa1 is selected from an amino acid of low amino acid hydropathy (e.g., Xaa1 is selected from K or R); or wherein Xaa1 is selected from an amino acid of high average amino acid flexibility index (e.g., Xaa1 is selected from D, E, R, K, G, I, N, Q, or S); or wherein Xaa1 is selected from an amino acid of high hydrogen bond donors (e.g., Xaa1 is selected from K or R); or wherein Xaa1 is selected from an amino acid of amino acid mutability (e.g., Xaa1 is selected from K, R, P, or H); or wherein Xaa2 is selected from an amino acid of low amino acid solubility (e.g., Xaa2 is selected from R, K, Q, or S); or wherein Xaa2 is selected from an amino acid of low amino acid hydropathy (e.g., Xaa2 is selected from R, K, D, E, N, Q, H, P, Y, W, S, or T); or wherein Xaa2 is selected from an amino acid of high amino acid charge (e.g., Xaa2 is selected from R, K, or H); or wherein Xaa3 is selected from an amino acid of high amino acid solubility (e.g., Xaa3 is selected from A, M, V, W, L, or I); or wherein Xaa5 is selected from an amino acid of high amino acid solubility (e.g., Xaa5 is selected from C, M, V, W, L, or I); or wherein Xaa5 is selected from an amino acid of high hydropathy (e.g., Xaa5 is selected from M, V, or I); or wherein Xaa5 is selected from an amino acid of low average amino acid flexibility index (e.g., Xaa5 is selected from M, W, F, or C); or wherein Xaa8 is selected from an amino acid of high amino acid solubility (e.g., Xaa8 is selected from H, V, or I); or any combination thereof.

In some embodiments, Xaa1 is selected from an amino acid of low amino acid solubility. In some embodiments, Xaa1 is selected from K, R, Q. In some embodiments, Xaa1 is selected from an amino acid of low amino acid hydropathy. In some embodiments, Xaa1 is selected from K or R. In some embodiments, Xaa1 is selected from an amino acid of high average amino acid flexibility index. In some embodiments, Xaa1 is selected from D, E, R, K, G, I, N, Q, or S. In some embodiments, Xaa1 is selected from an amino acid of high hydrogen bond donors. In some embodiments, Xaa1 is selected from K or R. In some embodiments, Xaa1 is selected from an amino acid of amino acid mutability. In some embodiments, Xaa1 is selected from K, R, P, or H. In some embodiments, Xaa2 is selected from an amino acid of low amino acid solubility. In some embodiments, Xaa2 is selected from R, K, Q, or S. In some embodiments, Xaa2 is selected from an amino acid of low amino acid hydropathy. In some embodiments, Xaa2 is selected from R, K, D, E, N, Q, H, P, Y, W, S, or T. In some embodiments, Xaa2 is selected from an amino acid of high amino acid charge. In some embodiments, Xaa2 is selected from R, K, H. In some embodiments, Xaa3 is selected from an amino acid of high amino acid solubility. In some embodiments, Xaa3 is selected from A, M, V, W, L, or I. In some embodiments, Xaa5 is selected from an amino acid of high amino acid solubility. In some embodiments, Xaa5 is selected from C, M, V, W, L, or I. In some embodiments, Xaa5 is selected from an amino acid of high hydropathy. In some embodiments, Xaa5 is selected from M, V, or I. In some embodiments, Xaa5 is selected from an amino acid of low average amino acid flexibility index. In some embodiments, Xaa5 is selected from M, W, F, or C. In some embodiments, Xaa8 is selected from an amino acid of high amino acid solubility. In some embodiments, Xaa8 is selected from H, V, or I.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 8118-SEQ ID NO: 9117, wherein said at least one mutation drives increased CNS tissue tropism.

### C. Enriched CNS Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 9118-SEQ ID NO: 10117, wherein said at least one mutation drives increased CNS tissue tropism.

6.7.7. In Vivo Selected Mutated VP Polypeptides that Confer Increased Spleen Tropism

The present disclosure provides AAV5 virions with a VP capsid polypeptide having at least one mutation in a region with residues that interact with target cells (e.g., a target spleen cell in a target spleen tissue of interest), where the at least one mutation confers increased spleen tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased spleen tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in spleen over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), liver, skeletal muscle, heart, lung, lymph node, bone marrow, mammary gland, skin, adrenal gland, thyroid, colon, sciatic nerve, and spinal cord tissues) was analyzed to identify a set of sequence rules for capsids that preferentially target spleen tissue. Identification of positional frequency rules from in vivo data is described in detail in EXAMPLE 6.

Disclosed herein are engineered AAV5 VP capsid polypeptides capable of forming an assembled virion that exhibits increased spleen tissue tropism as compared to wildtype AAV5 VP capsid polypeptide, wherein the engineered variant AAV5 VP1 capsid polypeptide sequence has one or more mutations, wherein Xaa1 is selected from C, F, H, I, L, P, W, or Y, or Xaa1 is selected from C, F, P, W, or Y, or Xaa1 is selected from P, W, or Y, or Xaa1 is P; or Xaa2 is selected from D, E, L, N, P, R, or W, or Xaa2 is selected from D, E, or W, or Xaa2 is D; or Xaa3 is selected from C, D, E, P, or W, or Xaa3 is selected from D, P, or W, or Xaa3 is P; or Xaa4 is selected from C, F, G, H, R, W or Y, or Xaa4 is selected from C, H, or W, or Xaa4 is C; or Xaa5 is selected from A, D, E, G, P, R, or W, or Xaa5 is selected from D, E, G, or P, or Xaa5 is D; or Xaa6 is selected from A, C, D, E, K, R, W, or Xaa6 is selected from C, K, or R, or Xaa6 is K; or Xaa7 is selected from F, L, P, R, W, Y, or Xaa7 is selected from L, P, or W, or Xaa7 is P; or Xaa8 is selected from E, I, K, L, P, R, or T, or Xaa8 is selected from P, R, or K, or Xaa8 is K; or Xaa9 is selected from C, H, M, T, V, or W, or Xaa9 is selected from C, T, or V, or Xaa9 is V.

Disclosed herein are engineered AAV5 VP capsid polypeptides capable of forming an assembled virion that exhibits increased spleen tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from C, F, H, I, L, P, W, or Y. In some embodiments, Xaa1 is selected from C, F, P, W, or Y. In some embodiments, Xaa1 is selected from P, W, or Y. In some embodiments, Xaa1 is P. In some embodiments, Xaa2 is selected from D, E, L, N, P, R, or W. In some embodiments, Xaa2 is selected from D, E, or W. In some embodiments, Xaa2 is D. In some embodiments, Xaa3 is selected from C, D, E, P, or W. In some embodiments, Xaa3 is selected from D, P, or W. In some embodiments, Xaa3 is P. In some embodiments, Xaa4 is selected from C, F, G, H, R, W or Y. In some embodiments, Xaa4 is selected from C, H, or W. In some embodiments, Xaa4 is C. In some embodiments, Xaa5 is selected from A, D, E, G, P, R, or W. In some embodiments, Xaa5 is selected from D, E, G, or P. In some embodiments, Xaa5 is D. In some embodiments, Xaa6 is selected from A, C, D, E, K, R, W. In some embodiments, Xaa6 is selected from C, K, or R. In some embodiments, Xaa6 is K. In some embodiments, Xaa7 is selected from F, L, P, R, W, Y. In some embodiments, Xaa7 is selected from L, P, or W. In some embodiments, Xaa7 is P. In some embodiments, Xaa8 is selected from E, I, K, L, P, R, or T. In some embodiments, Xaa8 is selected from P, R, or K. In some embodiments, Xaa8 is K. In some embodiments, Xaa9 is selected from C, H, M, T, V, or W. In some embodiments, Xaa9 is selected from C, T, or V. In some embodiments, Xaa9 is V.

Disclosed herein are engineered AAV5 VP capsid polypeptides capable of forming an assembled virion that exhibits increased spleen tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from C, F, H, I, L, P, W, or Y, Xaa2 is selected from D, E, L, N, P, R, or W, Xaa3 is selected from C, D, E, P, or W, Xaa4 is selected from C, F, G, H, R, W or Y, Xaa5 is selected from A, D, E, G, P, R, or W, Xaa6 is selected from A, C, D, E, K, R, W, Xaa7 is selected from F, L, P, R, W, Y, Xaa8 is selected from E, I, K, L, P, R, or T, and Xaa9 is selected from C, H, M, T, V, or W.

Disclosed herein are engineered AAV5 VP capsid polypeptides capable of forming an assembled virion that exhibits increased spleen tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from C, F, H, I, L, P, W, or Y, Xaa2 is selected from D, E, L, N, P, R, or W, Xaa3 is selected from C, D, E, P, or W, Xaa4 is selected from C, F, G, H, R, W or Y, Xaa5 is selected from A, D, E, G, P, R, or W, Xaa6 is selected from A, C, D, E, K, R, W, Xaa7 is selected from F, L, P, R, W, Y, Xaa8 is selected from E, I, K, L, P, R, or T, Xaa9 is selected from C, H, M, T, V, or W, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 37438-SEQ ID NO: 38437, wherein said at least one mutation drives increased spleen tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 42. Disclosed herein are engineered AAV5 VP capsid polypeptides capable of forming an assembled virion that exhibits increased spleen tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of low solubility (e.g., Xaa1 is selected from D or P); or wherein Xaa1 is selected from an amino acid of high solubility (e.g., Xaa1 is selected from F, I, L); or wherein Xaa1 is selected from an amino acid of low hydropathy (e.g., Xaa1 is selected from Y or P); or wherein Xaa1 is selected from an amino acid of low mutability (e.g., Xaa1 is selected from C, K, or P); or wherein Xaa2 is selected from an amino acid of low solubility (e.g., Xaa2 is selected from D, Q, or R); or wherein Xaa2 is selected from an amino acid of low hydropathy (e.g., Xaa2 is selected from D, E, R, K, H, N, or Q); or wherein Xaa2 is selected from an amino acid of low charge (e.g., Xaa2 is selected from D or E); or wherein Xaa2 is selected from an amino acid of low volume (e.g., Xaa2 is selected from T, N, P, or D); or wherein Xaa2 is selected from an amino acid of high average flexibility (e.g., Xaa2 is selected from D, E, R, P, G, Q, or S); or wherein Xaa3 is selected from an amino acid of low solubility (e.g., Xaa3 is selected from D, E, P, or N); or wherein Xaa3 is selected from an amino acid of low hydropathy (e.g., Xaa3 is selected from D, E, H, N, Q, or P); or wherein Xaa4 is selected from an amino acid of low hydropathy (e.g., Xaa4 is selected from K or R); or wherein Xaa5 is selected from an amino acid of low solubility (e.g., Xaa5 is selected from D, E, P, or N); or wherein Xaa5 is selected from an amino acid of high average flexibility (e.g., Xaa5 is selected from D, E, R, P, G, Q, or S); or wherein Xaa6 is selected from an amino acid of low mutability (e.g., Xaa6 is selected from C); or wherein Xaa8 is selected from an amino acid of high surface accessibility (e.g., Xaa8 is selected from E, R, or K); or wherein Xaa8 is selected from an amino acid of low solubility (e.g., Xaa8 is selected from E, P, R, K, N, or Q); or wherein Xaa8 is selected from an amino acid of medium volume (e.g., Xaa8 is selected from E, D, R, K, V, P, M, I, L, H, N, Q, or T); or wherein Xaa9 is selected from an amino acid of medium mol mass (e.g., Xaa9 is selected from E, D, K, M, I, L, H, or N); or any combination thereof.

In some embodiments, Xaa1 is selected from an amino acid of low solubility. In some embodiments, Xaa1 is selected from D or P. In some embodiments, Xaa1 is selected from an amino acid of high solubility. In some embodiments, Xaa1 is selected from F, I, or L. In some embodiments, Xaa1 is selected from an amino acid of low hydropathy. In some embodiments, Xaa1 is selected from Y or P. In some embodiments, Xaa1 is selected from an amino acid of low mutability. In some embodiments, Xaa1 is selected from C, K, or P. In some embodiments, Xaa2 is selected from an amino acid of low solubility. In some embodiments, Xaa2 is selected from D, Q, or R. In some embodiments, Xaa2 is selected from an amino acid of low hydropathy. In some embodiments, Xaa2 is selected from D, E, R, K, H, N, or Q. In some embodiments, Xaa2 is selected from an amino acid of low charge. In some embodiments, Xaa2 is selected from D or E. In some embodiments, Xaa2 is selected from an amino acid of low volume. In some embodiments, Xaa2 is selected from T, N, P, or D. In some embodiments, Xaa2 is selected from an amino acid of high average flexibility. In some embodiments, Xaa2 is selected from D, E, R, P, G, Q, or S. In some embodiments, Xaa3 is selected from an amino acid of low solubility. In some embodiments, Xaa3 is selected from D, E, P, or N. In some embodiments, Xaa3 is selected from an amino acid of low hydropathy. In some embodiments, Xaa3 is selected from D, E, H, N, Q, or P. In some embodiments, Xaa4 is selected from an amino acid of low hydropathy. In some embodiments, Xaa4 is selected from K or R. In some embodiments, Xaa5 is selected from an amino acid of low solubility. In some embodiments, Xaa5 is selected from D, E, P, or N. In some embodiments, Xaa5 is selected from an amino acid of high average flexibility. In some embodiments, Xaa5 is selected from D, E, R, P, G, Q, or S. In some embodiments, Xaa6 is selected from an amino acid of low mutability. In some embodiments, Xaa6 is selected from C. In some embodiments, Xaa8 is selected from an amino acid of high surface accessibility. In some embodiments, Xaa8 is selected from E, R, or K. In some embodiments, Xaa8 is selected from an amino acid of low solubility. In some embodiments, Xaa8 is selected from E, P, R, K, N, or Q. In some embodiments, Xaa8 is selected from an amino acid of medium volume. In some embodiments, Xaa8 is selected from E, D, R, K, V, P, M, I, L, H, N, Q, or T. In some embodiments, Xaa9 is selected from an amino acid of medium mol mass. In some embodiments, Xaa9 is selected from E, D, K, M, I, L, H, or N.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 38438-SEQ ID NO: 39437, wherein said at least one mutation drives increased spleen tissue tropism.

### C. Enriched Spleen Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 39438-SEQ ID NO: 40437, wherein said at least one mutation drives increased spleen tissue tropism.

6.7.8. In Vivo Selected Mutated VP Polypeptides that Confer Increased Adrenal Gland Tropism

The present disclosure provides AAV5 virions with a VP capsid polypeptide having at least one mutation in a region with residues that interact with target cells (e.g., a target adrenal gland cell in a target adrenal gland tissue of interest), where the at least one mutation confers increased adrenal gland tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased adrenal gland tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in adrenal gland over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), liver, skeletal muscle, heart, lung, spleen, lymph node, bone marrow, mammary gland, skin, thyroid, colon, sciatic nerve, and spinal cord tissues) was analyzed to identify a set of sequence rules for capsids that preferentially target adrenal gland tissue. Identification of positional frequency rules from in vivo data is described in detail in EXAMPLE 7.

Disclosed herein are engineered AAV5 VP capsid polypeptides capable of forming an assembled virion that exhibits increased adrenal gland tissue tropism as compared to wildtype AAV5 VP capsid polypeptide, wherein the engineered variant AAV5 VP1 capsid polypeptide sequence has one or more mutations, wherein Xaa1 is selected from A, C, K, Q, R, S, or T, or Xaa1 is selected from C, K, or R, or Xaa1 is C; or Xaa2 is selected from A, C, I, S, T, or V, or Xaa2 is selected from A, V, or T, or Xaa2 is V; or Xaa3 is selected from A, F, G, K, M, Q, R, T, or V, or Xaa3 is selected from A, G, or M, or Xaa3 is M; or Xaa4 is selected from A, K, M, Q, R, or V, or Xaa4 is selected from A, R, or K, or Xaa4 is K; or Xaa5 is selected from F, I, L, M, R, T, V, or Y, or Xaa5 is selected from R, V, or Y, or Xaa5 is V; or Xaa6 is selected from G, H, M, N, R, or S, or Xaa6 is selected from H or N, or Xaa6 is N; or Xaa7 is selected from A, H, K, Q, R, S or V, or Xaa7 is selected from H, Q, or V, or Xaa7 is H; or Xaa8 is selected from A, G, H, M, Q, or S, or Xaa8 is selected from A, G, M, or S, or Xaa8 is S; or Xaa9 is selected from A, E, N, P, R, S, or Y, or Xaa9 is selected from P or E, or Xaa9 is P.

Disclosed herein are engineered AAV5 VP capsid polypeptides capable of forming an assembled virion that exhibits increased adrenal gland tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from A, C, K, Q, R, S, or T. In some embodiments, Xaa1 is selected from C, K, or R. In some embodiments, Xaa1 is C. In some embodiments, Xaa2 is selected from A, C, I, S, T, or V. In some embodiments, Xaa2 is selected from A, V, or T. In some embodiments, Xaa2 is V. In some embodiments, Xaa3 is selected from A, F, G, K, M, Q, R, T, or V. In some embodiments, Xaa3 is selected from A, G, or M. In some embodiments, Xaa3 is M. In some embodiments, Xaa4 is selected from A, K, M, Q, R, or V. In some embodiments, Xaa4 is selected from A, R, or K. In some embodiments, Xaa4 is K. In some embodiments, Xaa5 is selected from F, I, L, M, R, T, V, or Y. In some embodiments, Xaa5 is selected from R, V, or Y. In some embodiments, Xaa5 is V. In some embodiments, Xaa6 is selected from G, H, M, N, R, or S. In some embodiments, Xaa6 is selected from H or N. In some embodiments, Xaa6 is N. In some embodiments, Xaa7 is selected from A, H, K, Q, R, S or V. In some embodiments, Xaa7 is selected from H, Q, or V. In some embodiments, Xaa7 is H. In some embodiments, Xaa8 is selected from A, G, H, M, Q, or S. In some embodiments, Xaa8 is selected from A, G, M, or S. In some embodiments, Xaa8 is S. In some embodiments, Xaa9 is selected from A, E, N, P, R, S, or Y. In some embodiments, Xaa9 is selected from P or E. In some embodiments, Xaa9 is P.

Disclosed herein are engineered AAV5 VP capsid polypeptides capable of forming an assembled virion that exhibits increased adrenal gland tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from A, C, K, Q, R, S, or T, Xaa2 is selected from A, C, I, S, T, or V, Xaa3 is selected from A, F, G, K, M, Q, R, T, or V, Xaa4 is selected from A, K, M, Q, R, or V, Xaa5 is selected from F, I, L, M, R, T, V, or Y, Xaa6 is selected from G, H, M, N, R, or S, Xaa7 is selected from A, H, K, Q, R, S or V, Xaa8 is selected from A, G, H, M, Q, or S, and, Xaa9 is selected from A, E, N, P, R, S, or Y.

Disclosed herein are engineered AAV5 VP capsid polypeptides capable of forming an assembled virion that exhibits increased adrenal gland tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from A, C, K, Q, R, S, or T, Xaa2 is selected from A, C, I, S, T, or V, Xaa3 is selected from A, F, G, K, M, Q, R, T, or V, Xaa4 is selected from A, K, M, Q, R, or V, Xaa5 is selected from F, I, L, M, R, T, V, or Y, Xaa6 is selected from G, H, M, N, R, or S, Xaa7 is selected from A, H, K, Q, R, S or V, Xaa8 is selected from A, G, H, M, Q, or S, Xaa9 is selected from A, E, N, P, R, S, or Y, or any combination thereof.

In some embodiments, provided herein are AAV5 VP capsid polypeptide having at least one mutation in a region from a position corresponding to 581 to a position corresponding to 589 and having at least 70%, at least 75%, at least 80%, at least 85%, at least 90%, at least 92%, at least 95%, at least 97%, at least 99%, or 100% sequence identity to any sequence selected SEQ ID NO: 1118-SEQ ID NO: 2117, wherein said at least one mutation drives increased adrenal gland tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 31. Disclosed herein are engineered AAV5 VP capsid polypeptides capable of forming an assembled virion that exhibits increased adrenal gland tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of low mol mass at Xaa1 (e.g., Xaa1 is selected from V, P, S, or C); or wherein Xaa1 is selected from an amino acid of low hydropathy (e.g., Xaa1 is selected from T, S, W, or Y); or wherein Xaa2 is selected from an amino acid of low hydropathy (e.g., Xaa2 is selected from R); or wherein Xaa2 is selected from an amino acid of low mutability (e.g., Xaa2 is selected from C); or wherein Xaa2 is selected from an amino acid of low solubility (e.g., Xaa2 is selected from K); or wherein Xaa3 is selected from an amino acid of low average flexibility (e.g., Xaa3 is selected from W, M, or F); or wherein Xaa3 is selected from an amino acid of high solubility (e.g., Xaa3 is selected from M); or wherein Xaa4 is selected from an amino acid of high surface accessibility (e.g., Xaa4 is selected from K or R); or wherein Xaa4 is selected from an amino acid of high average flexibility (e.g., Xaa4 is selected from K, I, or N); or wherein Xaa5 is selected from an amino acid of medium mutability (e.g., Xaa5 is selected from R or H); or wherein Xaa5 is selected from an amino acid of high goldman engelman steitz (e.g., Xaa5 is selected from V or L); or wherein Xaa5 is selected from an amino acid of low hydropathy (e.g., Xaa5 is selected from R); or wherein Xaa5 is selected from an amino acid of high volume (e.g., Xaa5 is selected from Y, R, or F); or wherein Xaa6 is selected from an amino acid of high solubility (e.g., Xaa6 is selected from Y, V, M, A, or C); or wherein Xaa7 is selected from an amino acid of medium mutability (e.g., Xaa7 is selected from V, H, or R); or wherein Xaa7 is selected from an amino acid of low solubility (e.g., Xaa7 is selected from R); or wherein Xaa8 is selected from an amino acid of high average flexibility (e.g., Xaa8 is selected from K, I, or N); or wherein Xaa8 is selected from an amino acid of high mol mass (e.g., Xaa8 is selected from R or Y); or wherein Xaa9 is selected from an amino acid of high mutability (e.g., Xaa9 is selected from N); or any combination thereof.

In some embodiments, Xaa1 is selected from an amino acid of low mol mass. In some embodiments, Xaa1 is selected from V, P, S, or C. In some embodiments, Xaa1 is selected from an amino acid of low hydropathy. In some embodiments, Xaa1 is selected from T, S, W, or Y. In some embodiments, Xaa2 is selected from an amino acid of low hydropathy. In some embodiments, Xaa2 is selected from R. In some embodiments, Xaa2 is selected from an amino acid of low mutability. In some embodiments, Xaa2 is selected from C. In some embodiments, Xaa2 is selected from an amino acid of low solubility. In some embodiments, Xaa2 is selected from K. In some embodiments, Xaa3 is selected from an amino acid of low average flexibility. In some embodiments, Xaa3 is selected from W, M, or F. In some embodiments, Xaa3 is selected from an amino acid of high solubility. In some embodiments, Xaa3 is selected from M. In some embodiments, Xaa4 is selected from an amino acid of high surface accessibility. In some embodiments, Xaa4 is selected from K or R. In some embodiments, Xaa4 is selected from an amino acid of high average flexibility. In some embodiments, Xaa4 is selected from K, I, or N. In some embodiments, Xaa5 is selected from an amino acid of medium mutability. In some embodiments, Xaa5 is selected from R, H. In some embodiments, Xaa5 is selected from an amino acid of high goldman engelman steitz. In some embodiments, Xaa5 is selected from V, L. In some embodiments, Xaa5 is selected from an amino acid of low hydropathy. In some embodiments, Xaa5 is selected from R. In some embodiments, Xaa5 is selected from an amino acid of high volume. In some embodiments, Xaa5 is selected from Y, R, or F. In some embodiments, Xaa6 is selected from an amino acid of high solubility. In some embodiments, Xaa6 is selected from Y, V, M, A, or C. In some embodiments, Xaa7 is selected from an amino acid of medium mutability. In some embodiments, Xaa7 is selected from V, H, or R. In some embodiments, Xaa7 is selected from an amino acid of low solubility. In some embodiments, Xaa7 is selected from R. In some embodiments, Xaa8 is selected from an amino acid of high average flexibility. In some embodiments, Xaa8 is selected from K, I, or N. In some embodiments, Xaa8 is selected from an amino acid of high mol mass. In some embodiments, Xaa8 is selected from R or Y. In some embodiments, Xaa9 is selected from an amino acid of high mutability. In some embodiments, Xaa9 is selected from N.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 2118-SEQ ID NO: 3117, wherein said at least one mutation drives increased adrenal gland tissue tropism.

### C. Enriched Adrenal Gland Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 3118-SEQ ID NO: 4117, wherein said at least one mutation drives increased adrenal gland tissue tropism.

6.7.9. In Vivo Selected Mutated VP Polypeptides that Confer Increased Sciatic Nerve Tropism

The present disclosure provides AAV5 virions with a VP capsid polypeptide having at least one mutation in a region with residues that interact with target cells (e.g., a target sciatic nerve cell in a target sciatic nerve tissue of interest), where the at least one mutation confers increased sciatic nerve tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased sciatic nerve tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in sciatic nerve over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), liver, skeletal muscle, heart, lung, spleen, lymph node, bone marrow, mammary gland, skin, adrenal gland, thyroid, colon, and spinal cord tissues) was analyzed to identify a set of sequence rules for capsids that preferentially target sciatic nerve tissue. Identification of positional frequency rules from in vivo data is described in detail in EXAMPLE 8.

Disclosed herein are engineered AAV5 VP capsid polypeptides capable of forming an assembled virion that exhibits increased sciatic nerve tissue tropism as compared to wildtype AAV5 VP capsid polypeptide, wherein the engineered variant AAV5 VP1 capsid polypeptide sequence has one or more mutations, wherein Xaa1 is selected from C, G, K, M, Q, R, or Y, or Xaa1 is selected from C, R, or Q, or Xaa1 is C; or Xaa2 is selected from A, C, F, I, Q, T, or V, or Xaa2 is selected from A, C, or I, or Xaa2 is A; or Xaa3 is selected from A, F, I, M, R, S, or T, or Xaa3 is selected from F, M, R, or S, or Xaa3 is R; or Xaa4 is selected from E, N, T, Q, or V, or Xaa4 is selected from E, T, or V, or Xaa4 is T; or Xaa5 is selected from F, H, Q, S, V, or Y, or Xaa5 is selected from F, V, or Y, or Xaa5 is V; or Xaa6 is selected from K, M, N, Q, S, or V, or Xaa6 is selected from M, N, or S, or Xaa6 is N; or Xaa7 is selected from K, M, Q, R, or T, or Xaa7 is selected from M, Q, or T, or Xaa7 is M; or Xaa8 is selected from A, G, H, Q, S, or V, or Xaa8 is selected from H or S, or Xaa8 is H; or Xaa9 is selected from C, E, I, K, or R, or Xaa9 is selected from C, I, or K, or Xaa9 is I.

Herein are engineered AAV5 VP capsid polypeptides capable of forming an assembled virion that exhibits increased sciatic nerve tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from C, G, K, M, Q, R, or Y. In some embodiments, Xaa1 is selected from C, R, or Q. In some embodiments, Xaa1 is C. In some embodiments, Xaa2 is selected from A, C, F, I, Q, T, or V. In some embodiments, Xaa2 is selected from A, C, or I. In some embodiments, Xaa2 is A. In some embodiments, Xaa3 is selected from A, F, I, M, R, S, or T. In some embodiments, Xaa3 is selected from F, M, R, or S. In some embodiments, Xaa3 is R. In some embodiments, Xaa4 is selected from E, N, T, Q, or V. In some embodiments, Xaa4 is selected from E, T, or V. In some embodiments, Xaa4 is T. In some embodiments, Xaa5 is selected from F, H, Q, S, V, or Y. In some embodiments, Xaa5 is selected from F, V, or Y. In some embodiments, Xaa5 is V. In some embodiments, Xaa6 is selected from K, M, N, Q, S, or V. In some embodiments, Xaa6 is selected from M, N, or S. In some embodiments, Xaa6 is N. In some embodiments, Xaa7 is selected from K, M, Q, R, or T. In some embodiments, Xaa7 is selected from M, Q, or T. In some embodiments, Xaa7 is M. In some embodiments, Xaa8 is selected from A, G, H, Q, S, or V. In some embodiments, Xaa8 is selected from H or S. In some embodiments, Xaa8 is H. In some embodiments, Xaa9 is selected from C, E, I, K, or R. In some embodiments, Xaa9 is selected from C, I, or K. In some embodiments, Xaa9 is I.

Disclosed herein are engineered AAV5 VP capsid polypeptides capable of forming an assembled virion that exhibits increased sciatic nerve tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from C, G, K, M, Q, R, or Y, Xaa2 is selected from A, C, F, I, Q, T, or V, Xaa3 is selected from A, F, I, M, R, S, or T, Xaa4 is selected from E, N, T, Q, or V, Xaa5 is selected from F, H, Q, S, V, or Y, Xaa6 is selected from K, M, N, Q, S, or V, Xaa7 is selected from K, M, Q, R, or T, Xaa8 is selected from A, G, H, Q, S, or V, and Xaa9 is selected from C, E, I, K, or R.

Disclosed herein are engineered AAV5 VP capsid polypeptides capable of forming an assembled virion that exhibits increased sciatic nerve tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from C, G, K, M, Q, R, or Y, Xaa2 is selected from A, C, F, I, Q, T, or V, Xaa3 is selected from A, F, I, M, R, S, or T, Xaa4 is selected from E, N, T, Q, or V, Xaa5 is selected from F, H, Q, S, V, or Y, Xaa6 is selected from K, M, N, Q, S, or V, Xaa7 is selected from K, M, Q, R, or T, Xaa8 is selected from A, G, H, Q, S, or V, Xaa9 is selected from C, E, I, K, or R, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 26118-SEQ ID NO: 26990, wherein said at least one mutation drives increased sciatic nerve tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 38. Disclosed herein are engineered AAV5 VP capsid polypeptides capable of forming an assembled virion that exhibits increased sciatic nerve tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of high average flexibility (e.g., Xaa1 is selected from G or R); or wherein Xaa1 is selected from an amino acid of low solubility (e.g., Xaa1 is selected from R, or Q); or wherein Xaa1 is selected from an amino acid of low mutability (e.g., Xaa1 is selected from C, L, F, Y, R, K, P, or H); or wherein Xaa1 is selected from an amino acid of high volume (e.g., Xaa1 is selected from Y, F); or wherein Xaa2 is selected from an amino acid of high surface accessibility (e.g., Xaa2 is selected from E, R, or K); or wherein Xaa3 is selected from an amino acid of medium mutability (e.g., Xaa3 is selected from H or R); or wherein Xaa3 is selected from an amino acid of medium average flexibility (e.g., Xaa3 is selected from V or Y); or wherein Xaa4 is selected from an amino acid of high mutability (e.g., Xaa4 is selected from N); or wherein Xaa4 is selected from an amino acid of high average flexibility (e.g., Xaa4 is selected from I, N, G, or R); or wherein Xaa4 is selected from an amino acid of low solubility (e.g., Xaa4 is selected from N); or wherein Xaa6 is selected from an amino acid of low mutability (e.g., Xaa6 is selected from C, L, F, or Y); or wherein Xaa6 is selected from an amino acid of high volume (e.g., Xaa6 is selected from K, M, I, or L); or wherein Xaa7 is selected from an amino acid of low mutability (e.g., Xaa7 is selected from L, F, or Y); or wherein Xaa7 is selected from an amino acid of medium mol mass (e.g., Xaa7 is selected from D, I, L, or N); or wherein Xaa8 is selected from an amino acid of high surface accessibility (e.g., Xaa8 is selected from S, Y, T, D, P, H, or N); or wherein Xaa9 is selected from an amino acid of low mutability (e.g., Xaa9 is selected from C, H, R); or wherein Xaa9 is selected from an amino acid of medium solubility (e.g., Xaa9 is selected from Q, T, or C); or wherein Xaa9 is selected from an amino acid of low surface accessibility (e.g., Xaa9 is selected from C); or any combination thereof.

In some embodiments, Xaa1 is selected from an amino acid of high average flexibility. In some embodiments, Xaa1 is selected from G or R. In some embodiments, Xaa1 is selected from an amino acid of low solubility. In some embodiments, Xaa1 is selected from R or Q. In some embodiments, Xaa1 is selected from an amino acid of low mutability. In some embodiments, Xaa1 is selected from C, L, F, Y, R, K, P, or H. In some embodiments, Xaa1 is selected from an amino acid of high volume. In some embodiments, Xaa1 is selected from Y or F. In some embodiments, Xaa2 is selected from an amino acid of high surface accessibility. In some embodiments, Xaa2 is selected from E, R, or K. In some embodiments, Xaa3 is selected from an amino acid of medium mutability. In some embodiments, Xaa3 is selected from H or R. In some embodiments, Xaa3 is selected from an amino acid of medium average flexibility. In some embodiments, Xaa3 is selected from V or Y. In some embodiments, Xaa4 is selected from an amino acid of high mutability. In some embodiments, Xaa4 is selected from N. In some embodiments, Xaa4 is selected from an amino acid of high average flexibility. In some embodiments, Xaa4 is selected from I, N, G, or R. In some embodiments, Xaa4 is selected from an amino acid of low solubility. In some embodiments, Xaa4 is selected from N. In some embodiments, Xaa6 is selected from an amino acid of low mutability. In some embodiments, Xaa6 is selected from C, L, F, or Y. In some embodiments, Xaa6 is selected from an amino acid of high volume. In some embodiments, Xaa6 is selected from K, M, I, or L. In some embodiments, Xaa7 is selected from an amino acid of low mutability. In some embodiments, Xaa7 is selected from L, F, or Y. In some embodiments, Xaa7 is selected from an amino acid of medium mol mass. In some embodiments, Xaa7 is selected from D, I, L, or N. In some embodiments, Xaa8 is selected from an amino acid of high surface accessibility. In some embodiments, Xaa8 is selected from S, Y, T, D, P, H, or N. In some embodiments, Xaa9 is selected from an amino acid of low mutability. In some embodiments, Xaa9 is selected from C, H, or R. In some embodiments, Xaa9 is selected from an amino acid of medium solubility. In some embodiments, Xaa9 is selected from Q, T, or C. In some embodiments, Xaa9 is selected from an amino acid of low surface accessibility. In some embodiments, Xaa9 is selected from C.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 26991-SEQ ID NO: 27990, wherein said at least one mutation drives increased sciatic nerve tissue tropism.

### C. Enriched Sciatic Nerve Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 27991-SEQ ID NO: 28990, wherein said at least one mutation drives increased sciatic nerve tissue tropism.

6.7.10. In Vivo Selected Mutated VP Polypeptides that Confer Increased Skeletal Muscle Tropism

The present disclosure provides AAV5 virions with a VP capsid polypeptide having at least one mutation in a region with residues that interact with target cells (e.g., a target skeletal muscle cell in a target skeletal muscle tissue of interest), where the at least one mutation confers increased skeletal muscle tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased skeletal muscle tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in skeletal muscle over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), liver, heart, lung, spleen, lymph node, bone marrow, mammary gland, skin, adrenal gland, thyroid, colon, sciatic nerve, and spinal cord tissues) was analyzed to identify a set of sequence rules for capsids that preferentially target skeletal muscle tissue. Identification of positional frequency rules from in vivo data is described in detail in EXAMPLE 9.

Disclosed herein are engineered AAV5 VP capsid polypeptides capable of forming an assembled virion that exhibits increased skeletal muscle tissue tropism as compared to wildtype AAV5 VP capsid polypeptide, wherein the engineered variant AAV5 VP1 capsid polypeptide sequence has one or more mutations, wherein Xaa1 is selected from A, E, H, M, P, Q, or S, or Xaa1 is selected from P or Q, or Xaa1 is Q; or Xaa2 is selected from F, H, I, T, or V, or Xaa2 is selected from T or V, or Xaa2 is V; or Xaa3 is selected from A, G, I, K, M, Q, R, S, T, or V, or Xaa3 is selected from A, L, P, R, or T, or Xaa3 is selected from L, P, or T, or Xaa3 is P; or Xaa4 is selected from D, E, G, P, or S, or Xaa4 is selected from D, E, or S, or Xaa4 is E; or Xaa5 is selected from H, L, M, P, or V, or Xaa5 is selected from L, M, or V, or Xaa5 is L; or Xaa6 is selected from E, H, N, or P, or Xaa6 is P; or Xaa7 is selected from A, H, N, Q or T, or Xaa7 is H; or Xaa8 is selected from I, K, M, P, or W, or Xaa8 is selected from I, P, or W, or Xaa8 is P; or Xaa9 is selected from A, I, M, P, or V, or Xaa9 is selected from A, M, or P, or Xaa9 is M.

Disclosed herein are engineered AAV5 VP capsid polypeptides capable of forming an assembled virion that exhibits increased skeletal muscle tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from A, E, H, M, P, Q, or S. In some embodiments, Xaa1 is selected from P or Q. In some embodiments, Xaa1 is Q. In some embodiments, Xaa2 is selected from F, H, I, T, or V. In some embodiments, Xaa2 is selected from T or V. In some embodiments, Xaa2 is V. In some embodiments, Xaa3 is selected from A, G, I, K, M, Q, R, S, T, or V. In some embodiments, Xaa3 is selected from A, L, P, R, or T. In some embodiments, Xaa3 is selected from L, P, or T. In some embodiments, Xaa3 is P. In some embodiments, Xaa4 is selected from D, E, G, P, or S. In some embodiments, Xaa4 is selected from D, E, or S. In some embodiments, Xaa4 is E. In some embodiments, Xaa5 is selected from H, L, M, P, or V. In some embodiments, Xaa5 is selected from L, M, or V. In some embodiments, Xaa5 is L. In some embodiments, Xaa6 is selected from E, H, N, or P. In some embodiments, Xaa6 is P. In some embodiments, Xaa7 is selected from A, H, N, Q or T. In some embodiments, Xaa7 is H. In some embodiments, Xaa8 is selected from I, K, M, P, or W. In some embodiments, Xaa8 is selected from I, P, or W. In some embodiments, Xaa8 is P. In some embodiments, Xaa9 is selected from A, I, M, P, or V. In some embodiments, Xaa9 is selected from A, M, or P. In some embodiments, Xaa9 is M.

Disclosed herein are engineered AAV5 VP capsid polypeptides capable of forming an assembled virion that exhibits increased skeletal muscle tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from A, E, H, M, P, Q, or S, Xaa2 is selected from F, H, I, T, or V, Xaa3 is selected from A, G, I, K, M, Q, R, S, T, or V, Xaa4 is selected from D, E, G, P, or S, Xaa5 is selected from H, L, M, P, or V, Xaa6 is selected from E, H, N, or P, Xaa7 is selected from A, H, N, Q or T, Xaa8 is selected from I, K, M, P, or W, and Xaa9 is selected from A, I, M, P, or V.

Disclosed herein are engineered AAV5 VP capsid polypeptides capable of forming an assembled virion that exhibits increased skeletal muscle tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from A, E, H, M, P, Q, or S, Xaa2 is selected from F, H, I, T, or V, Xaa3 is selected from A, G, I, K, M, Q, R, S, T, or V, Xaa4 is selected from D, E, G, P, or S, Xaa5 is selected from H, L, M, P, or V, Xaa6 is selected from E, H, N, or P, Xaa7 is selected from A, H, N, Q or T, Xaa8 is selected from I, K, M, P, or W, Xaa9 is selected from A, I, M, P, or V, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 28991-SEQ ID NO: 29990, wherein said at least one mutation drives increased skeletal muscle tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 39. Disclosed herein are engineered AAV5 VP capsid polypeptides capable of forming an assembled virion that exhibits increased skeletal muscle tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of high average flexibility (e.g., Xaa1 is selected from G or R); or wherein Xaa1 is selected from an amino acid of low average flexibility (e.g., Xaa1 is selected from W, M, F, or H); or wherein Xaa1 is selected from an amino acid of high mol mass (e.g., Xaa1 is selected from R, F, or W); or wherein Xaa2 is selected from an amino acid of low hydropathy (e.g., Xaa2 is selected from K, or R); or wherein Xaa2 is selected from an amino acid of low mutability (e.g., Xaa2 is selected from C, R, or H); or wherein Xaa2 is selected from an amino acid of high average flexibility (e.g., Xaa2 is selected from G or R); or wherein Xaa3 is selected from an amino acid of high average flexibility (e.g., Xaa3 is selected from G or R); or wherein Xaa4 is selected from an amino acid of high hydrophilicity (e.g., Xaa4 is selected from D, E, R, K, or N); or wherein Xaa4 is selected from an amino acid of low mutability (e.g., Xaa4 is selected from C, R, H); or wherein Xaa5 is selected from an amino acid of low mol mass (e.g., Xaa5 is selected from A); or wherein Xaa5 is selected from an amino acid of low average flexibility (e.g., Xaa5 is selected from A or L); or wherein Xaa5 is selected from an amino acid of high mutability (e.g., Xaa5 is selected from D, A, or E); or wherein Xaa6 is selected from an amino acid of low average flexibility (e.g., Xaa6 is selected from W, M, or F); or wherein Xaa6 is selected from an amino acid of low mutability (e.g., Xaa6 is selected from C); or wherein Xaa6 is selected from an amino acid of high mol mass (e.g., Xaa6 is selected from W); or wherein Xaa7 is selected from an amino acid of low goldman engelman steitz (e.g., Xaa7 is selected from R); or wherein Xaa7 is selected from an amino acid of high average flexibility (e.g., Xaa7 is selected from D, R, P, G, or S); or wherein Xaa7 is selected from an amino acid of high mutability (e.g., Xaa7 is selected from R, H, or N); or wherein Xaa7 is selected from an amino acid of low solubility (e.g., Xaa7 is selected from R or Q); or wherein Xaa8 is selected from an amino acid of high hydrophilicity (e.g., Xaa8 is selected from D, E, R, K, or N); or wherein Xaa9 is selected from an amino acid of low mutability (e.g., Xaa9 is selected from Y, F, or L); or any combination thereof.

In some embodiments, Xaa1 is selected from an amino acid of high average flexibility. In some embodiments, Xaa1 is selected from G or R. In some embodiments, Xaa1 is selected from an amino acid of low average flexibility. In some embodiments, Xaa1 is selected from W, M, F, or H. In some embodiments, Xaa1 is selected from an amino acid of high mol mass. In some embodiments, Xaa1 is selected from R, F, or W. In some embodiments, Xaa2 is selected from an amino acid of low hydropathy. In some embodiments, Xaa2 is selected from K or R. In some embodiments, Xaa2 is selected from an amino acid of low mutability. In some embodiments, Xaa2 is selected from C, R, or H. In some embodiments, Xaa2 is selected from an amino acid of high average flexibility. In some embodiments, Xaa2 is selected from G or R. In some embodiments, Xaa3 is selected from an amino acid of high average flexibility. In some embodiments, Xaa3 is selected from G or R. In some embodiments, Xaa4 is selected from an amino acid of high hydrophilicity. In some embodiments, Xaa4 is selected from D, E, R, K, or N. In some embodiments, Xaa4 is selected from an amino acid of low mutability. In some embodiments, Xaa4 is selected from C, R, or H. In some embodiments, Xaa5 is selected from an amino acid of low mol mass. In some embodiments, Xaa5 is selected from A. In some embodiments, Xaa5 is selected from an amino acid of low average flexibility. In some embodiments, Xaa5 is selected from A or L. In some embodiments, Xaa5 is selected from an amino acid of high mutability. In some embodiments, Xaa5 is selected from D, A, or E. In some embodiments, Xaa6 is selected from an amino acid of low average flexibility. In some embodiments, Xaa6 is selected from W, M, or F. In some embodiments, Xaa6 is selected from an amino acid of low mutability. In some embodiments, Xaa6 is selected from C. In some embodiments, Xaa6 is selected from an amino acid of high mol mass. In some embodiments, Xaa6 is selected from W. In some embodiments, Xaa7 is selected from an amino acid of low goldman engelman steitz. In some embodiments, Xaa7 is selected from R. In some embodiments, Xaa7 is selected from an amino acid of high average flexibility. In some embodiments, Xaa7 is selected from D, R, P, G, or S. In some embodiments, Xaa7 is selected from an amino acid of high mutability. In some embodiments, Xaa7 is selected from R, H, or N. In some embodiments, Xaa7 is selected from an amino acid of low solubility. In some embodiments, Xaa7 is selected from R or Q. In some embodiments, Xaa8 is selected from an amino acid of high hydrophilicity. In some embodiments, Xaa8 is selected from D, E, R, K, or N. In some embodiments, Xaa9 is selected from an amino acid of low mutability. In some embodiments, Xaa9 is selected from Y, F, or L.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 29991-SEQ ID NO: 30990, wherein said at least one mutation drives increased skeletal muscle tissue tropism.

### C. Enriched Skeletal Muscle Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 30991-SEQ ID NO: 31990, wherein said at least one mutation drives increased skeletal muscle tissue tropism.

6.7.11. In Vivo Selected Mutated VP Polypeptides that Confer Increased Spinal Cord Tropism

The present disclosure provides AAV5 virions with a VP capsid polypeptide having at least one mutation in a region with residues that interact with target cells (e.g., a target spinal cord cell in a target spinal cord tissue of interest), where the at least one mutation confers increased CNS tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased spinal cord tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in spinal cord over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), liver, skeletal muscle, heart, lung, spleen, lymph node, bone marrow, mammary gland, skin, adrenal gland, thyroid, colon, and sciatic nerve tissues) was analyzed to identify a set of sequence rules for capsids that preferentially target spinal cord tissue. Identification of positional frequency rules from in vivo data is described in detail in EXAMPLE 10.

Disclosed herein are engineered AAV5 VP capsid polypeptides capable of forming an assembled virion that exhibits increased spinal cord tissue tropism as compared to wildtype AAV5 VP capsid polypeptide, wherein the engineered variant AAV5 VP1 capsid polypeptide sequence has one or more mutations, wherein Xaa1 is selected from A, C, K, Q, R, S, or W, or Xaa1 is selected from K, R, or W, or Xaa1 is K; or Xaa2 is selected from H, I, K, L, T, V, or W, or Xaa2 is selected from H, I, or T, or Xaa2 is I; or Xaa3 is selected from C, F, G, H, I, K, N, or R, or Xaa3 is selected from F, I, or R, or Xaa3 is I; or Xaa4 is selected from I, M, Q, S, or V, or Xaa4 is selected from I, M, or V, or Xaa4 is V; or Xaa5 is selected from H, K, Q, T, W, or Y, or Xaa5 is selected from T, W, or Y, or Xaa5 is Y; or Xaa6 is selected from H, L, N, Q, R, W, or Y, or Xaa6 is selected from L, N, R, or Y, or Xaa6 is Y; or Xaa7 is selected from D, H, P, Q, or R, or Xaa7 is R; or Xaa8 is selected from D, F, L, S, T, or Y, or Xaa8 is selected from S, T, or Y, or Xaa8 is T; or Xaa9 is selected from C, I, N, P, R, S, or Y, or Xaa9 is selected from I, P, or R, or Xaa9 is I.

Disclosed herein are engineered AAV5 VP capsid polypeptides capable of forming an assembled virion that exhibits increased spinal cord tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from A, C, K, Q, R, S, or W. In some embodiments, Xaa1 is selected from K, R, or W. In some embodiments, Xaa1 is K. In some embodiments, Xaa2 is selected from H, I, K, L, T, V, or W. In some embodiments, Xaa2 is selected from H, I, or T. In some embodiments, Xaa2 is I. In some embodiments, Xaa3 is selected from C, F, G, H, I, K, N, or R. In some embodiments, Xaa3 is selected from F, I, or R. In some embodiments, Xaa3 is I. In some embodiments, Xaa4 is selected from I, M, Q, S, or V. In some embodiments, Xaa4 is selected from I, M, or V. In some embodiments, Xaa4 is V. In some embodiments, Xaa5 is selected from H, K, Q, T, W, or Y. In some embodiments, Xaa5 is selected from T, W, or Y. In some embodiments, Xaa5 is Y. In some embodiments, Xaa6 is selected from H, L, N, Q, R, W, or Y. In some embodiments, Xaa6 is selected from L, N, R, or Y. In some embodiments, Xaa6 is Y. In some embodiments, Xaa7 is selected from D, H, P, Q, or R. In some embodiments, Xaa7 is R. In some embodiments, Xaa8 is selected from D, F, L, S, T, or Y. In some embodiments, Xaa8 is selected from S, T, or Y. In some embodiments, Xaa8 is T. In some embodiments, Xaa9 is selected from C, I, N, P, R, S, or Y. In some embodiments, Xaa9 is selected from I, P, or R. In some embodiments, Xaa9 is I.

Disclosed herein are engineered AAV5 VP capsid polypeptides capable of forming an assembled virion that exhibits increased spinal cord tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from A, C, K, Q, R, S, or W, Xaa2 is selected from H, I, K, L, T, V, or W, Xaa3 is selected from C, F, G, H, I, K, N, or R, Xaa4 is selected from I, M, Q, S, or V, Xaa5 is selected from H, K, Q, T, W, or Y, Xaa6 is selected from H, L, N, Q, R, W, or Y, Xaa7 is selected from D, H, P, Q, or R, Xaa8 is selected from D, F, L, S, T, or Y, and Xaa9 is selected from C, I, N, P, R, S, or Y.

Disclosed herein are engineered AAV5 VP capsid polypeptides capable of forming an assembled virion that exhibits increased spinal cord tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from A, C, K, Q, R, S, or W, Xaa2 is selected from H, I, K, L, T, V, or W, Xaa3 is selected from C, F, G, H, I, K, N, or R, Xaa4 is selected from I, M, Q, S, or V, Xaa5 is selected from H, K, Q, T, W, or Y, Xaa6 is selected from H, L, N, Q, R, W, or Y, Xaa7 is selected from D, H, P, Q, or R, Xaa8 is selected from D, F, L, S, T, or Y, Xaa9 is selected from C, I, N, P, R, S, or Y, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 34991-SEQ ID NO: 35437, wherein said at least one mutation drives increased spinal cord tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 41. Disclosed herein are engineered AAV5 VP capsid polypeptides capable of forming an assembled virion that exhibits increased spinal cord tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of high volume (e.g., Xaa1 is selected from F, W, or Y); or wherein Xaa1 is selected from an amino acid of low mutability (e.g., Xaa1 is selected from Y, F, L, or C); or wherein Xaa1 is selected from an amino acid of high solubility (e.g., Xaa1 is selected from W, F, I, or L); or wherein Xaa1 is selected from an amino acid of low average flexibility (e.g., Xaa1 is selected from F, M, or W); or wherein Xaa2 is selected from an amino acid of low hydropathy (e.g., Xaa2 is selected from P or Y); or wherein Xaa3 is selected from an amino acid of low hydrophilicity (e.g., Xaa3 is selected from Y, W, V, M, F, I, or L); or wherein Xaa3 is selected from an amino acid of high solubility (e.g., Xaa3 is selected from W, F, I, or L); or wherein Xaa6 is selected from an amino acid of high volume (e.g., Xaa6 is selected from W, R, K, M, I, or L); or wherein Xaa6 is selected from an amino acid of high mol mass (e.g., Xaa6 is selected from W); or wherein Xaa8 is selected from an amino acid of high mol mass (e.g., Xaa8 is selected from W, E, K, M, H, or Q); or wherein Xaa8 is selected from an amino acid of high volume (e.g., Xaa8 is selected from W, K, M, I, or L); or wherein Xaa8 is selected from an amino acid of high goldman engelman steitz (e.g., Xaa8 is selected from V or L); or wherein Xaa9 is selected from an amino acid of high hydropathy (e.g., Xaa9 is selected from V, or I); or wherein Xaa9 is selected from an amino acid of high solubility (e.g., Xaa9 is selected from W, F, I, or L); or any combination thereof.

In some embodiments, Xaa1 is selected from an amino acid of high volume. In some embodiments, Xaa1 is selected from F, W, or Y. In some embodiments, Xaa1 is selected from an amino acid of low mutability. In some embodiments, Xaa1 is selected from Y, F, L, or C. In some embodiments, Xaa1 is selected from an amino acid of high solubility. In some embodiments, Xaa1 is selected from W, F, I, or L. In some embodiments, Xaa1 is selected from an amino acid of low average flexibility. In some embodiments, Xaa1 is selected from F, M, or W. In some embodiments, Xaa2 is selected from an amino acid of low hydropathy. In some embodiments, Xaa2 is selected from P or Y. In some embodiments, Xaa3 is selected from an amino acid of low hydrophilicity. In some embodiments, Xaa3 is selected from Y, W, V, M, F, I, or L. In some embodiments, Xaa3 is selected from an amino acid of high solubility. In some embodiments, Xaa3 is selected from W, F, I, L. In some embodiments, Xaa6 is selected from an amino acid of high volume. In some embodiments, Xaa6 is selected from W, R, K, M, I, or L. In some embodiments, Xaa6 is selected from an amino acid of high mol mass. In some embodiments, Xaa6 is selected from W. In some embodiments, Xaa8 is selected from an amino acid of high mol mass. In some embodiments, Xaa8 is selected from W, E, K, M, H, or Q. In some embodiments, Xaa8 is selected from an amino acid of high volume. In some embodiments, Xaa8 is selected from W, K, M, I, L. In some embodiments, Xaa8 is selected from an amino acid of high goldman engelman steitz. In some embodiments, Xaa8 is selected from V or L. In some embodiments, Xaa9 is selected from an amino acid of high hydropathy. In some embodiments, Xaa9 is selected from V or I. In some embodiments, Xaa9 is selected from an amino acid of high solubility. In some embodiments, Xaa9 is selected from W, F, I, or L.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 35438-SEQ ID NO: 36437, wherein said at least one mutation drives increased spinal cord tissue tropism.

### C. Enriched Spinal Cord Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 36438-SEQ ID NO: 37437, wherein said at least one mutation drives increased spinal cord tissue tropism.

6.7.12. In Vivo Selected Mutated VP Polypeptides that Confer Increased Mammary Gland Tropism

The present disclosure provides AAV5 virions with a VP capsid polypeptide having at least one mutation in a region with residues that interact with target cells (e.g., a target mammary gland cell in a target mammary gland tissue of interest), where the at least one mutation confers increased mammary gland tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased mammary gland tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in mammary gland over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), liver, skeletal muscle, heart, lung, spleen, lymph node, bone marrow, skin, adrenal gland, thyroid, colon, sciatic nerve, and spinal cord tissues) was analyzed to identify a set of sequence rules for capsids that preferentially target mammary gland tissue. Identification of positional frequency rules from in vivo data is described in detail in EXAMPLE 11.

Disclosed herein are engineered AAV5 VP capsid polypeptides capable of forming an assembled virion that exhibits increased mammary gland tissue tropism as compared to wildtype AAV5 VP capsid polypeptide, wherein the engineered variant AAV5 VP1 capsid polypeptide sequence has one or more mutations, wherein Xaa1 is selected from C, K, M, Q, R, or Y, or Xaa1 is selected from C, Q, or R, or Xaa1 is C; or Xaa2 is selected from A, F, I, K, S, T, or V, or Xaa2 is selected from A, S, or V, or Xaa2 is V; or Xaa3 is selected from A, F, G, I, K, L, R, T, or Y, or Xaa3 is selected from F, G, K, R, or Y, or Xaa3 is selected from F, K, or Y, or Xaa3 is F; or Xaa4 is selected from A, I, K, Q, R, or T, or Xaa4 is selected from A, I, or R, or Xaa4 is I; or Xaa5 is selected from I, L, M, Q, R, T, V, or Y, or Xaa5 is selected from I, M, or Y, or Xaa5 is Y; or Xaa6 is selected from H, N, S, or V, or Xaa6 is H; or Xaa7 is selected from A, H, I, N, S or Y, or Xaa7 is N or S, or Xaa7 is N; or Xaa8 is selected from A, C, D, G, H, M, Q, or S, or Xaa8 is selected from G, M, or Q, or Xaa8 is G; or Xaa9 is selected from A, E, L, W, or Y, or Xaa9 is selected from A, L, or W, or Xaa9 is A.

Disclosed herein are engineered AAV5 VP capsid polypeptides capable of forming an assembled virion that exhibits increased mammary gland tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from C, K, M, Q, R, or Y. In some embodiments, Xaa1 is selected from C, Q, or R. In some embodiments, Xaa1 is C. In some embodiments, Xaa2 is selected from A, F, I, K, S, T, or V. In some embodiments, Xaa2 is selected from A, S, or V. In some embodiments, Xaa2 is V. In some embodiments, Xaa3 is selected from A, F, G, I, K, L, R, T, or Y. In some embodiments, Xaa3 is selected from F, G, K, R, or Y. In some embodiments, Xaa3 is selected from F, K, or Y. In some embodiments, Xaa3 is F. In some embodiments, Xaa4 is selected from A, I, K, Q, R, or T. In some embodiments, Xaa4 is selected from A, I, or R. In some embodiments, Xaa4 is I. In some embodiments, Xaa5 is selected from I, L, M, Q, R, T, V, or Y. In some embodiments, Xaa5 is selected from I, M, or Y. In some embodiments, Xaa5 is Y. In some embodiments, Xaa6 is selected from H, N, S, or V. In some embodiments, Xaa6 is H. In some embodiments, Xaa7 is selected from A, H, I, N, S or Y. In some embodiments, Xaa7 is N or S. In some embodiments, Xaa7 is N. In some embodiments, Xaa8 is selected from A, C, D, G, H, M, Q, or S. In some embodiments, Xaa8 is selected from G, M, or Q. In some embodiments, Xaa8 is G. In some embodiments, Xaa9 is selected from A, E, L, W, or Y. In some embodiments, Xaa9 is selected from A, L, or W. In some embodiments, Xaa9 is A.

Disclosed herein are engineered AAV5 VP capsid polypeptides capable of forming an assembled virion that exhibits increased mammary gland tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from C, K, M, Q, R, or Y, Xaa2 is selected from A, F, I, K, S, T, or V, Xaa3 is selected from A, F, G, I, K, L, R, T, or Y, Xaa4 is selected from A, I, K, Q, R, or T, Xaa5 is selected from I, L, M, Q, R, T, V, or Y, Xaa6 is selected from H, N, S, or V, Xaa7 is selected from A, H, I, N, S or Y, Xaa8 is selected from A, C, D, G, H, M, Q, or S, and, Xaa9 is selected from A, E, L, W, or Y.

Disclosed herein are engineered AAV5 VP capsid polypeptides capable of forming an assembled virion that exhibits increased mammary gland tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from C, K, M, Q, R, or Y, Xaa2 is selected from A, F, I, K, S, T, or V, Xaa3 is selected from A, F, G, I, K, L, R, T, or Y, Xaa4 is selected from A, I, K, Q, R, or T, Xaa5 is selected from I, L, M, Q, R, T, V, or Y, Xaa6 is selected from H, N, S, or V, Xaa7 is selected from A, H, I, N, S or Y, Xaa8 is selected from A, C, D, G, H, M, Q, or S, Xaa9 is selected from A, E, L, W, or Y, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 22118-SEQ ID NO: 23117, wherein said at least one mutation drives increased mammary gland tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 36. Disclosed herein are engineered AAV5 VP capsid polypeptides capable of forming an assembled virion that exhibits increased mammary gland tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of low surface accessibility (e.g., Xaa1 is selected from C); or wherein Xaa1 is selected from an amino acid of medium mol mass (e.g., Xaa1 is selected from C); or wherein Xaa2 is selected from an amino acid of high surface accessibility (e.g., Xaa2 is selected from D, N, or Q); or wherein Xaa2 is selected from an amino acid of low hydropathy (e.g., Xaa2 is selected from D, E, R, K, H, N, or Q); or wherein Xaa3 is selected from an amino acid of high average flexibility (e.g., Xaa3 is selected from D, E, R, P, G, or S); or wherein Xaa3 is selected from an amino acid of medium mutability (e.g., Xaa3 is selected from R or H); or wherein Xaa4 is selected from an amino acid of high mutability (e.g., Xaa4 is selected from M, I, Q, or T); or wherein Xaa4 is selected from an amino acid of high solubility (e.g., Xaa4 is selected from W, F, I, or L); or wherein Xaa4 is selected from an amino acid of high surface accessibility (e.g., Xaa4 is selected from E, R, or K); or wherein Xaa5 is selected from an amino acid of high solubility (e.g., Xaa5 is selected from W, F, I, or L); or wherein Xaa5 is selected from an amino acid of low mutability (e.g., Xaa5 is selected from Y, F, or L); or wherein Xaa6 is selected from an amino acid of high hydropathy (e.g., Xaa6 is selected from V, I, or L); or wherein Xaa6 is selected from an amino acid of medium mol mass (e.g., Xaa6 is selected from D, I, L, or N); or wherein Xaa8 is selected from an amino acid of low surface accessibility (e.g., Xaa8 is selected from C); or wherein Xaa8 is selected from an amino acid of low mutability (e.g., Xaa8 is selected from C, R, or H); or wherein Xaa9 is selected from an amino acid of medium mutability (e.g., Xaa9 is selected from R or H); or any combination thereof.

In some embodiments, Xaa1 is selected from an amino acid of low surface accessibility. In some embodiments, Xaa1 is selected from C. In some embodiments, Xaa1 is selected from an amino acid of medium mol mass. In some embodiments, Xaa1 is selected from C. In some embodiments, Xaa2 is selected from an amino acid of high surface accessibility. In some embodiments, Xaa2 is selected from D, N, or Q. In some embodiments, Xaa2 is selected from an amino acid of low hydropathy. In some embodiments, Xaa2 is selected from D, E, R, K, H, N, or Q. In some embodiments, Xaa3 is selected from an amino acid of high average flexibility. In some embodiments, Xaa3 is selected from D, E, R, P, G, or S. In some embodiments, Xaa3 is selected from an amino acid of medium mutability. In some embodiments, Xaa3 is selected from R or H. In some embodiments, Xaa4 is selected from an amino acid of high mutability. In some embodiments, Xaa4 is selected from M, I, Q, or T. In some embodiments, Xaa4 is selected from an amino acid of high solubility. In some embodiments, Xaa4 is selected from W, F, I, or L. In some embodiments, Xaa4 is selected from an amino acid of high surface accessibility. In some embodiments, Xaa4 is selected from E, R, or K. In some embodiments, Xaa5 is selected from an amino acid of high solubility. In some embodiments, Xaa5 is selected from W, F, I, or L. In some embodiments, Xaa5 is selected from an amino acid of low mutability. In some embodiments, Xaa5 is selected from Y, F, or L. In some embodiments, Xaa6 is selected from an amino acid of high hydropathy. In some embodiments, Xaa6 is selected from V, I, or L. In some embodiments, Xaa6 is selected from an amino acid of medium mol mass. In some embodiments, Xaa6 is selected from D, I, L, or N. In some embodiments, Xaa8 is selected from an amino acid of low surface accessibility. In some embodiments, Xaa8 is selected from C. In some embodiments, Xaa8 is selected from an amino acid of low mutability. In some embodiments, Xaa8 is selected from C, R, or H. In some embodiments, Xaa9 is selected from an amino acid of medium mutability. In some embodiments, Xaa9 is selected from R or H.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 23118-SEQ ID NO: 24117, wherein said at least one mutation drives increased mammary gland tissue tropism.

### C. Enriched Mammary Gland Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 24118-SEQ ID NO: 25117, wherein said at least one mutation drives increased mammary gland tissue tropism.

6.7.13. In Vivo Selected Mutated VP Polypeptides that Confer Increased Lung Tropism

The present disclosure provides AAV5 virions with a VP capsid polypeptide having at least one mutation in a region with residues that interact with target cells (e.g., a target lung cell in a target lung tissue of interest), where the at least one mutation confers increased lung tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased lung tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in lung over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), liver, skeletal muscle, heart, spleen, lymph node, bone marrow, mammary gland, skin, adrenal gland, thyroid, colon, sciatic nerve, and spinal cord tissues) was analyzed to identify a set of sequence rules for capsids that preferentially target lung tissue. Identification of positional frequency rules from in vivo data is described in detail in EXAMPLE 12.

Disclosed herein are engineered AAV5 VP capsid polypeptides capable of forming an assembled virion that exhibits increased lung tissue tropism as compared to wildtype AAV5 VP capsid polypeptide, wherein the engineered variant AAV5 VP1 capsid polypeptide sequence has one or more mutations, wherein Xaa1 is selected from A, E, K, M, Q, R, S, or T, or Xaa1 is selected from A, E, or Q, or Xaa1 is E; or Xaa2 is selected from A, I, K, S, T, or V, or Xaa2 is selected from S, T, or V, or Xaa2 is T; or Xaa3 is selected from A, E, K, M, Q, R, S, T, or V, or Xaa3 is selected from A, K, R, or S, or Xaa3 is R; or Xaa4 is selected from M, P, R, S, or T, or Xaa4 is selected from P, Q, or T, or Xaa4 is Q; or Xaa5 is selected from I, K, L, M, T, V, or Y, or Xaa5 is selected from L, M, or Y, or Xaa5 is L; or Xaa6 is selected from D, G, H, M, N, R, or S, or Xaa6 is selected from H or N, or Xaa6 is N; or Xaa7 is selected from A, K, M, Q, or R, or Xaa7 is selected from A, K or R, or Xaa7 is R; or Xaa8 is selected from A, F, G, S, W, or Y, or Xaa8 is selected from A, F, or G, or Xaa8 is F; or Xaa9 is selected from A, E, G, P, R, or Y, or Xaa9 is selected from G, P, or R, or Xaa9 is G.

Disclosed herein are engineered AAV5 VP capsid polypeptides capable of forming an assembled virion that exhibits increased lung tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from A, E, K, M, Q, R, S, or T. In some embodiments, Xaa1 is selected from A, E, or Q. In some embodiments, Xaa1 is E. In some embodiments, Xaa2 is selected from A, I, K, S, T, or V. In some embodiments, Xaa2 is selected from S, T, or V. In some embodiments, Xaa2 is T. In some embodiments, Xaa3 is selected from A, E, K, M, Q, R, S, T, or V. In some embodiments, Xaa3 is selected from A, K, R, or S. In some embodiments, Xaa3 is R. In some embodiments, Xaa4 is selected from M, P, R, S, or T. In some embodiments, Xaa4 is selected from P, Q, or T. In some embodiments, Xaa4 is Q. In some embodiments, Xaa5 is selected from I, K, L, M, T, V, or Y. In some embodiments, Xaa5 is selected from L, M, or Y. In some embodiments, Xaa5 is L. In some embodiments, Xaa6 is selected from D, G, H, M, N, R, or S. In some embodiments, Xaa6 is selected from H or N. In some embodiments, Xaa6 is N. In some embodiments, Xaa7 is selected from A, K, M, Q, or R. In some embodiments, Xaa7 is selected from A, K or R. In some embodiments, Xaa7 is R. In some embodiments, Xaa8 is selected from A, F, G, S, W, or Y. In some embodiments, Xaa8 is selected from A, F, or G. In some embodiments, Xaa8 is F. In some embodiments, Xaa9 is selected from A, E, G, P, R, or Y. In some embodiments, Xaa9 is selected from G, P, or R. In some embodiments, Xaa9 is G.

Disclosed herein are engineered AAV5 VP capsid polypeptides capable of forming an assembled virion that exhibits increased lung tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from A, E, K, M, Q, R, S, or T, Xaa2 is selected from A, I, K, S, T, or V, Xaa3 is selected from A, E, K, M, Q, R, S, T, or V, Xaa4 is selected from M, P, R, S, or T, Xaa5 is selected from I, K, L, M, T, V, or Y, Xaa6 is selected from D, G, H, M, N, R, or S, Xaa7 is selected from A, K, M, Q, or R, Xaa8 is selected from A, F, G, S, W, or Y, and, Xaa9 is selected from A, E, G, P, R, or Y.

Disclosed herein are engineered AAV5 VP capsid polypeptides capable of forming an assembled virion that exhibits increased lung tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from A, E, K, M, Q, R, S, or T, Xaa2 is selected from A, I, K, S, T, or V, Xaa3 is selected from A, E, K, M, Q, R, S, T, or V, Xaa4 is selected from M, P, R, S, or T, Xaa5 is selected from I, K, L, M, T, V, or Y, Xaa6 is selected from D, G, H, M, N, R, or S, Xaa7 is selected from A, K, M, Q, or R, Xaa8 is selected from A, F, G, S, W, or Y, Xaa9 is selected from A, E, G, P, R, or Y, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 16118-SEQ ID NO: 17117, wherein said at least one mutation drives increased lung tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 37. Disclosed herein are engineered AAV5 VP capsid polypeptides capable of forming an assembled virion that exhibits increased lung tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of high mutability (e.g., Xaa1 is selected from D, E, M, A, I, Q, or T); or wherein Xaa2 is selected from an amino acid of high mol mass (e.g., Xaa2 is selected from F); or wherein Xaa2 is selected from an amino acid of low mutability (e.g., Xaa2 is selected from Y, F, or L); or wherein Xaa3 is selected from an amino acid of low mutability (e.g., Xaa3 is selected from K, V, P, or H); or wherein Xaa3 is selected from an amino acid of low hydropathy (e.g., Xaa3 is selected from K or R); or wherein Xaa4 is selected from an amino acid of low mutability (e.g., Xaa4 is selected from K or P); or wherein Xaa4 is selected from an amino acid of high average flexibility (e.g., Xaa4 is selected from D, E, P, or S); or wherein Xaa5 is selected from an amino acid of low average flexibility (e.g., Xaa5 is selected from W, M, or F); or wherein Xaa5 is selected from an amino acid of high solubility (e.g., Xaa5 is selected from W, F, I, or L); or wherein Xaa6 is selected from an amino acid of medium mutability (e.g., Xaa6 is selected from R or H); or wherein Xaa6 is selected from an amino acid of high surface accessibility (e.g., Xaa6 is selected from T); or wherein Xaa7 is selected from an amino acid of low mutability (e.g., Xaa7 is selected from C); or wherein Xaa7 is selected from an amino acid of high solubility (e.g., Xaa7 is selected from W, V, M, F, I, or L); or wherein Xaa8 is selected from an amino acid of high mutability (e.g., Xaa8 is selected from D, E, M, A, I, Q, or T); or wherein Xaa8 is selected from an amino acid of low hydropathy (e.g., Xaa8 is selected from R or K); or wherein Xaa9 is selected from an amino acid of high average flexibility (e.g., Xaa9 is selected from R or G); or any combination thereof.

In some embodiments, Xaa1 is selected from an amino acid of high mutability. In some embodiments, Xaa1 is selected from D, E, M, A, I, Q, or T. In some embodiments, Xaa2 is selected from an amino acid of high mol mass. In some embodiments, Xaa2 is selected from F. In some embodiments, Xaa2 is selected from an amino acid of low mutability. In some embodiments, Xaa2 is selected from Y, F, or L. In some embodiments, Xaa3 is selected from an amino acid of low mutability. In some embodiments, Xaa3 is selected from K, V, P, or H. In some embodiments, Xaa3 is selected from an amino acid of low hydropathy. In some embodiments, Xaa3 is selected from K or R. In some embodiments, Xaa4 is selected from an amino acid of low mutability. In some embodiments, Xaa4 is selected from K or P. In some embodiments, Xaa4 is selected from an amino acid of high average flexibility. In some embodiments, Xaa4 is selected from D, E, P, or S. In some embodiments, Xaa5 is selected from an amino acid of low average flexibility. In some embodiments, Xaa5 is selected from W, M, or F. In some embodiments, Xaa5 is selected from an amino acid of high solubility. In some embodiments, Xaa5 is selected from W, F, I, or L. In some embodiments, Xaa6 is selected from an amino acid of medium mutability. In some embodiments, Xaa6 is selected from R or H. In some embodiments, Xaa6 is selected from an amino acid of high surface accessibility. In some embodiments, Xaa6 is selected from T. In some embodiments, Xaa7 is selected from an amino acid of low mutability. In some embodiments, Xaa7 is selected from C. In some embodiments, Xaa7 is selected from an amino acid of high solubility. In some embodiments, Xaa7 is selected from W, V, M, F, I, or L. In some embodiments, Xaa8 is selected from an amino acid of high mutability. In some embodiments, Xaa8 is selected from D, E, M, A, I, Q, or T. In some embodiments, Xaa8 is selected from an amino acid of low hydropathy. In some embodiments, Xaa8 is selected from R or K. In some embodiments, Xaa9 is selected from an amino acid of high average flexibility. In some embodiments, Xaa9 is selected from R or G.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 17118-SEQ ID NO: 18117, wherein said at least one mutation drives increased lung tissue tropism.

### C. Enriched Lung Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 18118-SEQ ID NO: 19117, wherein said at least one mutation drives increased lung tissue tropism.

6.7.14. In Vivo Selected Mutated VP Polypeptides that Confer Increased Heart Tropism

The present disclosure provides AAV5 virions with a VP capsid polypeptide having at least one mutation in a region with residues that interact with target cells (e.g., a target heart cell in a target heart tissue of interest), where the at least one mutation confers increased heart tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased heart tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section. In some embodiments, “heart” and “cardiac” may be used interchangeably herein.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in heart over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), liver, skeletal muscle, lung, spleen, lymph node, bone marrow, mammary gland, skin, adrenal gland, thyroid, colon, sciatic nerve, and spinal cord tissues) was analyzed to identify a set of sequence rules for capsids that preferentially target heart tissue. Identification of positional frequency rules from in vivo data is described in detail in EXAMPLE 13.

Disclosed herein are engineered AAV5 VP capsid polypeptides capable of forming an assembled virion that exhibits increased heart tissue tropism as compared to wildtype AAV5 VP capsid polypeptide, wherein the engineered variant AAV5 VP capsid polypeptide sequence has one or more mutations, wherein Xaa1 is selected from I, K, L, M, T, or V, or Xaa1 is selected from K or L, or Xaa1 is K; or Xaa2 is selected from A, C, G, I, K, or S, or Xaa2 is selected from A, C, or S, or Xaa2 is A; or Xaa3 is selected from A, D, E, G, K, M, or V, or Xaa3 is selected from E or V, or Xaa3 is E; or Xaa4 is selected from F, H, R, T, W, or Y, or Xaa4 is selected from F, R, or T, or Xaa4 is R; or Xaa5 is selected from F, L, M, or R, or Xaa5 is L; or Xaa6 is selected from A, H, N, W, or Y, or Xaa6 is selected from H, N, or Y, or Xaa6 is H; or Xaa7 is selected from A, C, E, F, K, or T, or Xaa7 is selected from C, F, or T, or Xaa7 is F; or Xaa8 is selected from A, C, M, S, or T, or Xaa8 is selected from C, M, or S, or Xaa8 is C; or Xaa9 is selected from A, D, G, or P, or Xaa9 is selected from A or G, or Xaa9 is A.

Disclosed herein are engineered AAV5 VP capsid polypeptides capable of forming an assembled virion that exhibits increased heart tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from I, K, L, M, T, or V. In some embodiments, Xaa1 is selected from K or L. In some embodiments, Xaa1 is K. In some embodiments, Xaa2 is selected from A, C, G, I, K, or S. In some embodiments, Xaa2 is selected from A, C, or S. In some embodiments, Xaa2 is A. In some embodiments, Xaa3 is selected from A, D, E, G, K, M, or V. In some embodiments, Xaa3 is selected from E or V. In some embodiments, Xaa3 is E. In some embodiments, Xaa4 is selected from F, H, R, T, W, or Y. In some embodiments, Xaa4 is selected from F, R, or T. In some embodiments, Xaa4 is R. In some embodiments, Xaa5 is selected from F, L, M, or R. In some embodiments, Xaa5 is L. In some embodiments, Xaa6 is selected from A, H, N, W, or Y. In some embodiments, Xaa6 is selected from H, N, or Y. In some embodiments, Xaa6 is H. In some embodiments, Xaa7 is selected from A, C, E, F, K, or T. In some embodiments, Xaa7 is selected from C, F, or T. In some embodiments, Xaa7 is F. In some embodiments, Xaa8 is selected from A, C, M, S, or T. In some embodiments, Xaa8 is selected from C, M, or S. In some embodiments, Xaa8 is C. In some embodiments, Xaa9 is selected from A, D, G, or P. In some embodiments, Xaa9 is selected from A or G. In some embodiments, Xaa9 is A.

Disclosed herein are engineered AAV5 VP capsid polypeptides capable of forming an assembled virion that exhibits increased heart tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from I, K, L, M, T, or V, Xaa2 is selected from A, C, G, I, K, or S, Xaa3 is selected from A, D, E, G, K, M, or V, Xaa4 is selected from F, H, R, T, W, or Y, Xaa5 is selected from F, L, M, or R, Xaa6 is selected from A, H, N, W, or Y, Xaa7 is selected from A, C, E, F, K, or T, Xaa8 is selected from A, C, M, S, or T, and Xaa9 is selected from A, D, G, or P.

Disclosed herein are engineered AAV5 VP capsid polypeptides capable of forming an assembled virion that exhibits increased heart tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from I, K, L, M, T, or V, Xaa2 is selected from A, C, G, I, K, or S, Xaa3 is selected from A, D, E, G, K, M, or V, Xaa4 is selected from F, H, R, T, W, or Y, Xaa5 is selected from F, L, M, or R, Xaa6 is selected from A, H, N, W, or Y, Xaa7 is selected from A, C, E, F, K, or T, Xaa8 is selected from A, C, M, S, or T, Xaa9 is selected from A, D, G, or P, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 13118-SEQ ID NO: 14117, wherein said at least one mutation drives increased heart tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 34. Disclosed herein are engineered AAV5 VP capsid polypeptides capable of forming an assembled virion that exhibits increased heart tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of low solubility (e.g., Xaa1 is selected from N or E); or wherein Xaa1 is selected from an amino acid of low hydropathy (e.g., Xaa1 is selected from H, N, Q, P, Y, D, or E); or wherein Xaa1 is selected from an amino acid of high mutability (e.g., Xaa1 is selected from A or E); or wherein Xaa2 is selected from an amino acid of high hydropathy (e.g., Xaa2 is selected from V or I); or wherein Xaa2 is selected from an amino acid of medium mutability (e.g., Xaa2 is selected from V); or wherein Xaa2 is selected from an amino acid of medium volume (e.g., Xaa2 is selected from V, E, or Q); or wherein Xaa2 is selected from an amino acid of high solubility (e.g., Xaa2 is selected from V or M); or wherein Xaa3 is selected from an amino acid of low solubility (e.g., Xaa3 is selected from R or Q); or wherein Xaa4 is selected from an amino acid of low surface accessibility (e.g., Xaa4 is selected from C); or wherein Xaa4 is selected from an amino acid of high solubility (e.g., Xaa4 is selected from C); or wherein Xaa4 is selected from an amino acid of low charge (e.g., Xaa4 is selected from D, E, Y, W, V, P, M, A, G, F, I, L, N, Q, S, T, or C); or wherein Xaa4 is selected from an amino acid of high hydropathy (e.g., Xaa4 is selected from C); or wherein Xaa5 is selected from an amino acid of high surface accessibility (e.g., Xaa5 is selected from D, E, R, K, N, or Q); or wherein Xaa5 is selected from an amino acid of low solubility (e.g., Xaa5 is selected from D); or wherein Xaa6 is selected from an amino acid of low mutability (e.g., Xaa6 is selected from C); or wherein Xaa6 is selected from an amino acid of low solubility (e.g., Xaa6 is selected from D); or wherein Xaa8 is selected from an amino acid of high surface accessibility (e.g., Xaa8 is selected from D or N); or wherein Xaa8 is selected from an amino acid of high average flexibility (e.g., Xaa8 is selected from D, R, P, G, or S); or wherein Xaa9 is selected from an amino acid of medium mol mass (e.g., Xaa9 is selected from N, D, L, or I); or any combination thereof.

In some embodiments, Xaa1 is selected from an amino acid of low solubility. In some embodiments, Xaa1 is selected from N or E. In some embodiments, Xaa1 is selected from an amino acid of low hydropathy. In some embodiments, Xaa1 is selected from H, N, Q, P, Y, D, or E. In some embodiments, Xaa1 is selected from an amino acid of high mutability. In some embodiments, Xaa1 is selected from A or E. In some embodiments, Xaa2 is selected from an amino acid of high hydropathy. In some embodiments, Xaa2 is selected from V or I. In some embodiments, Xaa2 is selected from an amino acid of medium mutability. In some embodiments, Xaa2 is selected from V. In some embodiments, Xaa2 is selected from an amino acid of medium volume. In some embodiments, Xaa2 is selected from V, E, or Q. In some embodiments, Xaa2 is selected from an amino acid of high solubility. In some embodiments, Xaa2 is selected from V or M. In some embodiments, Xaa3 is selected from an amino acid of low solubility. In some embodiments, Xaa3 is selected from R or Q. In some embodiments, Xaa4 is selected from an amino acid of low surface accessibility. In some embodiments, Xaa4 is selected from C. In some embodiments, Xaa4 is selected from an amino acid of high solubility. In some embodiments, Xaa4 is selected from C. In some embodiments, Xaa4 is selected from an amino acid of low charge. In some embodiments, Xaa4 is selected from D, E, Y, W, V, P, M, A, G, F, I, L, N, Q, S, T, or C. In some embodiments, Xaa4 is selected from an amino acid of high hydropathy. In some embodiments, Xaa4 is selected from C. In some embodiments, Xaa5 is selected from an amino acid of high surface accessibility. In some embodiments, Xaa5 is selected from D, E, R, K, N, or Q. In some embodiments, Xaa5 is selected from an amino acid of low solubility. In some embodiments, Xaa5 is selected from D. In some embodiments, Xaa6 is selected from an amino acid of low mutability. In some embodiments, Xaa6 is selected from C. In some embodiments, Xaa6 is selected from an amino acid of low solubility. In some embodiments, Xaa6 is selected from D. In some embodiments, Xaa8 is selected from an amino acid of high surface accessibility. In some embodiments, Xaa8 is selected from D or N. In some embodiments, Xaa8 is selected from an amino acid of high average flexibility. In some embodiments, Xaa8 is selected from D, R, P, G, or S. In some embodiments, Xaa9 is selected from an amino acid of medium mol mass. In some embodiments, Xaa9 is selected from N, D, L, or I.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 14118-SEQ ID NO: 15117, wherein said at least one mutation drives increased heart tissue tropism.

### C. Enriched Heart Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 15118-SEQ ID NO: 16117, wherein said at least one mutation drives increased heart tissue tropism.

6.7.15. In Vivo Selected Mutated VP Polypeptides that Confer Increased Colon Tropism

The present disclosure provides AAV5 virions with a VP capsid polypeptide having at least one mutation in a region with residues that interact with target cells (e.g., a target colon cell in a target colon tissue of interest), where the at least one mutation confers increased colon tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased colon tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in colon over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), liver, skeletal muscle, heart, lung, spleen, lymph node, bone marrow, mammary gland, skin, adrenal gland, thyroid, sciatic nerve, and spinal cord tissues) was analyzed to identify a set of sequence rules for capsids that preferentially target colon tissue. Identification of positional frequency rules from in vivo data is described in detail in EXAMPLE 14.

Disclosed herein are engineered AAV5 VP capsid polypeptides capable of forming an assembled virion that exhibits increased colon tissue tropism as compared to wildtype AAV5 VP capsid polypeptide, wherein the engineered variant AAV5 VP1 capsid polypeptide sequence has one or more mutations, wherein Xaa1 is selected from C, F, H, N, P, W, or Y, or Xaa1 is selected from F, P, or W, or Xaa1 is P; or Xaa2 is selected from D, E, F, L, or P, or Xaa2 is selected from D, E, L, or P, or Xaa2 is P; or Xaa3 is selected from C, F, H, I, L, P, or Y, or Xaa3 is selected from C, H, or P, or Xaa3 is P; or Xaa4 is selected from C, D, E, N, or P, or Xaa4 is selected from C, D, or E, or Xaa4 is C; or Xaa5 is selected from D, E, G, P, or W, or Xaa5 is selected from G, P, or W, or Xaa5 is P; or Xaa6 is selected from C, K, R, or V, or Xaa6 is selected from K or R, or Xaa6 is R; or Xaa7 is selected from D, M, P, or V, or Xaa7 is P; or Xaa8 is selected from D, I, K, L, P, R, or V, or Xaa8 is selected from K, P, or R, or Xaa8 is P; or Xaa9 is selected from C, H, I, K, L, M, or W, or Xaa9 is selected from I, L, or M, or Xaa9 is I.

Disclosed herein are engineered AAV5 VP capsid polypeptides capable of forming an assembled virion that exhibits increased colon tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from C, F, H, N, P, W, or Y. In some embodiments, Xaa1 is selected from F, P, or W. In some embodiments, Xaa1 is P. In some embodiments, Xaa2 is selected from D, E, F, L, or P. In some embodiments, Xaa2 is selected from D, E, L, or P. In some embodiments, Xaa2 is P. In some embodiments, Xaa3 is selected from C, F, H, I, L, P, or Y. In some embodiments, Xaa3 is selected from C, H, or P. In some embodiments, Xaa3 is P. In some embodiments, Xaa4 is selected from C, D, E, N, or P. In some embodiments, Xaa4 is selected from C, D, or E. In some embodiments, Xaa4 is C. In some embodiments, Xaa5 is selected from D, E, G, P, or W. In some embodiments, Xaa5 is selected from G, P, or W. In some embodiments, Xaa5 is P. In some embodiments, Xaa6 is selected from C, K, R, or V. In some embodiments, Xaa6 is selected from K or R. In some embodiments, Xaa6 is R. In some embodiments, Xaa7 is selected from D, M, P, or V. In some embodiments, Xaa7 is P. In some embodiments, Xaa8 is selected from D, I, K, L, P, R, or V. In some embodiments, Xaa8 is selected from K, P, or R. In some embodiments, Xaa8 is P. In some embodiments, Xaa9 is selected from C, H, I, K, L, M, or W. In some embodiments, Xaa9 is selected from I, L, or M. In some embodiments, Xaa9 is I.

Disclosed herein are engineered AAV5 VP capsid polypeptides capable of forming an assembled virion that exhibits increased colon tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from C, F, H, N, P, W, or Y, Xaa2 is selected from D, E, F, L, or P, Xaa3 is selected from C, F, H, I, L, P, or Y, Xaa4 is selected from C, D, E, N, or P, Xaa5 is selected from D, E, G, P, or W, Xaa6 is selected from C, K, R, or V, Xaa7 is selected from D, M, P, or V, Xaa8 is selected from D, I, K, L, P, R, or V, and Xaa9 is selected from C, H, I, K, L, M, or W.

Disclosed herein are engineered AAV5 VP capsid polypeptides capable of forming an assembled virion that exhibits increased colon tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from C, F, H, N, P, W, or Y, Xaa2 is selected from D, E, F, L, or P, Xaa3 is selected from C, F, H, I, L, P, or Y, Xaa4 is selected from C, D, E, N, or P, Xaa5 is selected from D, E, G, P, or W, Xaa6 is selected from C, K, R, or V, Xaa7 is selected from D, M, P, or V, Xaa8 is selected from D, I, K, L, P, R, or V, Xaa9 is selected from C, H, I, K, L, M, or W, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 10118-SEQ ID NO: 11117, wherein said at least one mutation drives increased colon tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 33. Disclosed herein are engineered AAV5 VP capsid polypeptides capable of forming an assembled virion that exhibits increased colon tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of high mol mass (e.g., Xaa1 is selected from Y or W); or wherein Xaa1 is selected from an amino acid of high solubility (e.g., Xaa1 is selected from W, F, I, or L); or wherein Xaa2 is selected from an amino acid of low solubility (e.g., Xaa2 is selected from D); or wherein Xaa2 is selected from an amino acid of low mutability (e.g., Xaa2 is selected from P or K); or wherein Xaa2 is selected from an amino acid of medium mol mass (e.g., Xaa2 is selected from D, E, N, K, M, Q, I, or L); or wherein Xaa2 is selected from an amino acid of low hydropathy (e.g., Xaa2 is selected from D, E, R, K, H, N, or Q); or wherein Xaa3 is selected from an amino acid of low mutability (e.g., Xaa3 is selected from K, V, P, or C); or wherein Xaa3 is selected from an amino acid of high solubility (e.g., Xaa3 is selected from W, F, I, or L); or wherein Xaa5 is selected from an amino acid of high average flexibility (e.g., Xaa5 is selected from S, P, G, R, E, or D); or wherein Xaa5 is selected from an amino acid of high surface accessibility (e.g., Xaa5 is selected from D or N); or wherein Xaa6 is selected from an amino acid of low hydropathy (e.g., Xaa6 is selected from R); or wherein Xaa6 is selected from an amino acid of low mutability (e.g., Xaa6 is selected from Y, R, F, or L); or wherein Xaa6 is selected from an amino acid of low solubility (e.g., Xaa6 is selected from R or Q); or wherein Xaa6 is selected from an amino acid of high surface accessibility (e.g., Xaa6 is selected from E, R, or K); or wherein Xaa6 is selected from an amino acid of high average flexibility (e.g., Xaa6 is selected from G or R); or wherein Xaa8 is selected from an amino acid of low solubility (e.g., Xaa8 is selected from D); or any combination thereof.

In some embodiments, Xaa1 is selected from an amino acid of high mol mass. In some embodiments, Xaa1 is selected from Y or W. In some embodiments, Xaa1 is selected from an amino acid of high solubility. In some embodiments, Xaa1 is selected from W, F, I, or L. In some embodiments, Xaa2 is selected from an amino acid of low solubility. In some embodiments, Xaa2 is selected from D. In some embodiments, Xaa2 is selected from an amino acid of low mutability. In some embodiments, Xaa2 is selected from P, K. In some embodiments, Xaa2 is selected from an amino acid of medium mol mass. In some embodiments, Xaa2 is selected from D, E, N, K, M, Q, I, or L. In some embodiments, Xaa2 is selected from an amino acid of low hydropathy. In some embodiments, Xaa2 is selected from D, E, R, K, H, N, or Q. In some embodiments, Xaa3 is selected from an amino acid of low mutability. In some embodiments, Xaa3 is selected from K, V, P, or C. In some embodiments, Xaa3 is selected from an amino acid of high solubility. In some embodiments, Xaa3 is selected from W, F, I, or L. In some embodiments, Xaa5 is selected from an amino acid of high average flexibility. In some embodiments, Xaa5 is selected from S, P, G, R, E, or D. In some embodiments, Xaa5 is selected from an amino acid of high surface accessibility. In some embodiments, Xaa5 is selected from D or N. In some embodiments, Xaa6 is selected from an amino acid of low hydropathy. In some embodiments, Xaa6 is selected from R. In some embodiments, Xaa6 is selected from an amino acid of low mutability. In some embodiments, Xaa6 is selected from Y, R, F, or L. In some embodiments, Xaa6 is selected from an amino acid of low solubility. In some embodiments, Xaa6 is selected from R or Q. In some embodiments, Xaa6 is selected from an amino acid of high surface accessibility. In some embodiments, Xaa6 is selected from E, R, or K. In some embodiments, Xaa6 is selected from an amino acid of high average flexibility. In some embodiments, Xaa6 is selected from G or R. In some embodiments, Xaa8 is selected from an amino acid of low solubility. In some embodiments, Xaa8 is selected from D.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 11118-SEQ ID NO: 12117, wherein said at least one mutation drives increased colon tissue tropism.

### C. Enriched Colon Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 12118-SEQ ID NO: 13117, wherein said at least one mutation drives increased colon tissue tropism.

6.7.16. In Vivo Selected Mutated VP Polypeptides that Confer Increased Thyroid Gland Tropism

The present disclosure provides AAV5 virions with a VP capsid polypeptide having at least one mutation in a region with residues that interact with target cells (e.g., a target thyroid cell in a target thyroid gland tissue of interest), where the at least one mutation confers increased thyroid gland tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased thyroid gland tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in thyroid gland over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), liver, skeletal muscle, heart, lung, spleen, lymph node, bone marrow, mammary gland, skin, adrenal gland, colon, sciatic nerve, and spinal cord tissues) was analyzed to identify a set of sequence rules for capsids that preferentially target thyroid gland tissue. Identification of positional frequency rules from in vivo data is described in detail in EXAMPLE 15.

Disclosed herein are engineered AAV5 VP capsid polypeptides capable of forming an assembled virion that exhibits increased thyroid gland tissue tropism as compared to wildtype AAV5 VP capsid polypeptide, wherein the engineered variant AAV5 VP1 capsid polypeptide sequence has one or more mutations, wherein Xaa1 is selected from A, K, M, N, Q, or R, or Xaa1 is selected from K, N or Q, or Xaa1 is K; or Xaa2 is selected from A, F, K, L, M, T, V, or W, or Xaa2 is selected from F, V, or W, or Xaa2 is W; or Xaa3 is selected from A, I, K, R, S, T, V, or W, or Xaa3 is selected from A, R or T, or Xaa3 is R; or Xaa4 is selected from A, D, E, I, P, or V, or Xaa4 is selected from A, E, or I, or Xaa4 is A; or Xaa5 is selected from F, I, M, Q, V, or Y, or Xaa5 is M, V, Y, or Xaa5 is M; or Xaa6 is selected from H, M, N, or Y, or Xaa6 is N; or Xaa7 is selected from H, I, N, Q, S, or W, or Xaa7 is selected from H, I, or N, or Xaa7 is H; or Xaa8 is selected from A, D, F, Q, S, or Y, or Xaa8 is selected from A, F, or S, or Xaa8 is F; or Xaa9 is selected from A, Q, S, or Y, or Xaa9 is selected from A or S, or Xaa9 is A.

Disclosed herein are engineered AAV5 VP capsid polypeptides capable of forming an assembled virion that exhibits increased thyroid gland tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from A, K, M, N, Q, or R. In some embodiments, Xaa1 is selected from K, N or Q. In some embodiments, Xaa1 is K. In some embodiments, Xaa2 is selected from A, F, K, L, M, T, V, or W. In some embodiments, Xaa2 is selected from F, V, or W. In some embodiments, Xaa2 is W. In some embodiments, Xaa3 is selected from A, I, K, R, S, T, V, or W. In some embodiments, Xaa3 is selected from A, R or T. In some embodiments, Xaa3 is R. In some embodiments, Xaa4 is selected from A, D, E, I, P, or V. In some embodiments, Xaa4 is selected from A, E, or I. In some embodiments, Xaa4 is A. In some embodiments, Xaa5 is selected from F, I, M, Q, V, or Y. In some embodiments, Xaa5 is M, V, Y. In some embodiments, Xaa5 is M. In some embodiments, Xaa6 is selected from H, M, N, or Y. In some embodiments, Xaa6 is N. In some embodiments, Xaa7 is selected from H, I, N, Q, S, or W. In some embodiments, Xaa7 is selected from H, I, or N. In some embodiments, Xaa7 is H. In some embodiments, Xaa8 is selected from A, D, F, Q, S, or Y. In some embodiments, Xaa8 is selected from A, F, or S. In some embodiments, Xaa8 is F. In some embodiments, Xaa9 is selected from A, Q, S, or Y. In some embodiments, Xaa9 is selected from A or S. In some embodiments, Xaa9 is A.

Disclosed herein are engineered AAV5 VP capsid polypeptides capable of forming an assembled virion that exhibits increased thyroid gland tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from A, K, M, N, Q, or R, Xaa2 is selected from A, F, K, L, M, T, V, or W, Xaa3 is selected from A, I, K, R, S, T, V, or W, Xaa4 is selected from A, D, E, I, P, or V, Xaa5 is selected from F, I, M, Q, V, or Y, Xaa6 is selected from H, M, N, or Y, Xaa7 is selected from H, I, N, Q, S, or W, Xaa8 is selected from A, D, F, Q, S, or Y, and Xaa9 is selected from A, Q, S, or Y.

Disclosed herein are engineered AAV5 VP capsid polypeptides capable of forming an assembled virion that exhibits increased thyroid gland tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from A, K, M, N, Q, or R, Xaa2 is selected from A, F, K, L, M, T, V, or W, Xaa3 is selected from A, I, K, R, S, T, V, or W, Xaa4 is selected from A, D, E, I, P, or V, Xaa5 is selected from F, I, M, Q, V, or Y, Xaa6 is selected from H, M, N, or Y, Xaa7 is selected from H, I, N, Q, S, or W, Xaa8 is selected from A, D, F, Q, S, or Y, Xaa9 is selected from A, Q, S, or Y, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 40438-SEQ ID NO: 41437, wherein said at least one mutation drives increased thyroid gland tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 43. Disclosed herein are engineered AAV5 VP capsid polypeptides capable of forming an assembled virion that exhibits increased thyroid gland tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of high mutability (e.g., Xaa1 is selected from N); or wherein Xaa2 is selected from an amino acid of low surface accessibility (e.g., Xaa2 is selected from F, G, or M); or wherein Xaa3 is selected from an amino acid of high solubility (e.g., Xaa3 is selected from F); or wherein Xaa3 is selected from an amino acid of low mutability (e.g., Xaa3 is selected from Y, F, L, or C); or wherein Xaa3 is selected from an amino acid of medium mol mass (e.g., Xaa3 is selected from D, E, R, K, V, P, M, I, L, N, Q, T, or C); or wherein Xaa3 is selected from an amino acid of low surface accessibility (e.g., Xaa3 is selected from V, I, L, or C); or wherein Xaa4 is selected from an amino acid of high goldman engelman steitz (e.g., Xaa4 is selected from L or V); or wherein Xaa4 is selected from an amino acid of low surface accessibility (e.g., Xaa4 is selected from V, M, A, G, F, I, or L); or wherein Xaa4 is selected from an amino acid of low mol mass (e.g., Xaa4 is selected from D, A, G, I, L, or N); or wherein Xaa5 is selected from an amino acid of high solubility (e.g., Xaa5 is selected from C, L, F, M, V, or Y); or wherein Xaa5 is selected from an amino acid of low solubility (e.g., Xaa5 is selected from D); or wherein Xaa5 is selected from an amino acid of low average flexibility (e.g., Xaa5 is selected from F, M, or W); or wherein Xaa6 is selected from an amino acid of low average flexibility (e.g., Xaa6 is selected from F, M, or W); or wherein Xaa7 is selected from an amino acid of high mutability (e.g., Xaa7 is selected from N); or wherein Xaa7 is selected from an amino acid of low volume (e.g., Xaa7 is selected from P, N, or T); or wherein Xaa8 is selected from an amino acid of low average flexibility (e.g., Xaa8 is selected from F, M, or W); or wherein Xaa8 is selected from an amino acid of low surface accessibility (e.g., Xaa8 is selected from M, G, or F); or wherein Xaa9 is selected from an amino acid of low mutability (e.g., Xaa9 is selected from R, K, P, H, or C); or wherein Xaa9 is selected from an amino acid of low hydropathy (e.g., Xaa9 is selected from R); or any combination thereof.

In some embodiments, Xaa1 is selected from an amino acid of high mutability. In some embodiments, Xaa1 is selected from N. In some embodiments, Xaa2 is selected from an amino acid of low surface accessibility. In some embodiments, Xaa2 is selected from F, G, or M. In some embodiments, Xaa3 is selected from an amino acid of high solubility. In some embodiments, Xaa3 is selected from F. In some embodiments, Xaa3 is selected from an amino acid of low mutability. In some embodiments, Xaa3 is selected from Y, F, L, or C. In some embodiments, Xaa3 is selected from an amino acid of medium mol mass. In some embodiments, Xaa3 is selected from D, E, R, K, V, P, M, I, L, N, Q, T, or C. In some embodiments, Xaa3 is selected from an amino acid of low surface accessibility. In some embodiments, Xaa3 is selected from V, I, L, or C. In some embodiments, Xaa4 is selected from an amino acid of high goldman engelman steitz. In some embodiments, Xaa4 is selected from L or V. In some embodiments, Xaa4 is selected from an amino acid of low surface accessibility. In some embodiments, Xaa4 is selected from V, M, A, G, F, I, or L. In some embodiments, Xaa4 is selected from an amino acid of low mol mass. In some embodiments, Xaa4 is selected from D, A, G, I, L, or N. In some embodiments, Xaa5 is selected from an amino acid of high solubility. In some embodiments, Xaa5 is selected from C, L, F, M, V, or Y. In some embodiments, Xaa5 is selected from an amino acid of low solubility. In some embodiments, Xaa5 is selected from D. In some embodiments, Xaa5 is selected from an amino acid of low average flexibility. In some embodiments, Xaa5 is selected from F, M, or W. In some embodiments, Xaa6 is selected from an amino acid of low average flexibility. In some embodiments, Xaa6 is selected from F, M, or W. In some embodiments, Xaa7 is selected from an amino acid of high mutability. In some embodiments, Xaa7 is selected from N. In some embodiments, Xaa7 is selected from an amino acid of low volume. In some embodiments, Xaa7 is selected from P, N, or T. In some embodiments, Xaa8 is selected from an amino acid of low average flexibility. In some embodiments, Xaa8 is selected from F, M, or W. In some embodiments, Xaa8 is selected from an amino acid of low surface accessibility. In some embodiments, Xaa8 is selected from M, G, or F. In some embodiments, Xaa9 is selected from an amino acid of low mutability. In some embodiments, Xaa9 is selected from R, K, P, H, or C. In some embodiments, Xaa9 is selected from an amino acid of low hydropathy. In some embodiments, Xaa9 is selected from R.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 41438-SEQ ID NO: 42437, wherein said at least one mutation drives increased thyroid gland tissue tropism.

### C. Enriched Thyroid Gland Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 42438-SEQ ID NO: 43437, wherein said at least one mutation drives increased thyroid gland tissue tropism.

6.7.17. In Vivo Selected Mutated VP Polypeptides that Confer Increased Lymph Node Tropism

The present disclosure provides AAV5 virions with a VP capsid polypeptide having at least one mutation in a region with residues that interact with target cells (e.g., a target lymph node cell in a target lymph node tissue of interest), where the at least one mutation confers increased lymph node tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased lymph node tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in lymph node over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), liver, skeletal muscle, heart, lung, spleen, bone marrow, mammary gland, skin, adrenal gland, thyroid, colon, sciatic nerve, and spinal cord tissues) was analyzed to identify a set of sequence rules for capsids that preferentially target lymph node tissue. Identification of positional frequency rules from in vivo data is described in detail in EXAMPLE 16.

Disclosed herein are engineered AAV5 VP capsid polypeptides capable of forming an assembled virion that exhibits increased lymph node tissue tropism as compared to wildtype AAV5 VP capsid polypeptide, wherein the engineered variant AAV5 VP1 capsid polypeptide sequence has one or more mutations, wherein Xaa1 is selected from A, D, E, Q, S, or T, or Xaa1 is selected from D, E, or T, or Xaa1 is E; or Xaa2 is selected from A, H, I, S, T, or V, or Xaa2 is selected from I, T, or V, or Xaa2 is V; or Xaa3 is selected from A, E, H, I, T, or V, or Xaa3 is selected from A, I, T, or V, or Xaa3 is T; or Xaa4 is selected from A, D, E, or P, or Xaa4 is selected from D, or E, or Xaa4 is E; or Xaa5 is selected from I, L, M, V, or Y, or Xaa5 is selected from I, L, V, or Y, or Xaa5 is L; or Xaa6 is selected from D, E, I, N, or Q, or Xaa6 is selected from D, E, or I, or Xaa6 is D; or Xaa7 is selected from A, E, G, Q, or V, or Xaa7 is A, Q, or V, or Xaa7 is V; or Xaa8 is selected from F, G, M, or W, or Xaa8 is selected from For W, or Xaa8 is W; or Xaa9 is selected from I, P, T, or Y, or Xaa9 is I or P.

Disclosed herein are engineered AAV5 VP capsid polypeptides capable of forming an assembled virion that exhibits increased lymph node tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from A, D, E, Q, S, or T. In some embodiments, Xaa1 is selected from D, E, or T. In some embodiments, Xaa1 is E. In some embodiments, Xaa2 is selected from A, H, I, S, T, or V. In some embodiments, Xaa2 is selected from I, T, or V. In some embodiments, Xaa2 is V. In some embodiments, Xaa3 is selected from A, E, H, I, T, or V. In some embodiments, Xaa3 is selected from A, I, T, or V. In some embodiments, Xaa3 is T. In some embodiments, Xaa4 is selected from A, D, E, or P. In some embodiments, Xaa4 is selected from D, or E. In some embodiments, Xaa4 is E. In some embodiments, Xaa5 is selected from I, L, M, V, or Y. In some embodiments, Xaa5 is selected from I, L, V, or Y. In some embodiments, Xaa5 is L. In some embodiments, Xaa6 is selected from D, E, I, N, or Q. In some embodiments, Xaa6 is selected from D, E, or I. In some embodiments, Xaa6 is D. In some embodiments, Xaa7 is selected from A, E, G, Q, or V. In some embodiments, Xaa7 is A, Q, or V. In some embodiments, Xaa7 is V. In some embodiments, Xaa8 is selected from F, G, M, or W. In some embodiments, Xaa8 is selected from For W. In some embodiments, Xaa8 is W. In some embodiments, Xaa9 is selected from I, P, T, or Y. In some embodiments, Xaa9 is I or P.

Disclosed herein are engineered AAV5 VP capsid polypeptides capable of forming an assembled virion that exhibits increased lymph node tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from A, D, E, Q, S, or T, Xaa2 is selected from A, H, I, S, T, or V, Xaa3 is selected from A, E, H, I, T, or V, Xaa4 is selected from A, D, E, or P, Xaa5 is selected from I, L, M, V, or Y, Xaa6 is selected from D, E, I, N, or Q, Xaa7 is selected from A, E, G, Q, or V, Xaa8 is selected from F, G, M, or W, and Xaa9 is selected from I, P, T, or Y.

Disclosed herein are engineered AAV5 VP capsid polypeptides capable of forming an assembled virion that exhibits increased lymph node tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from A, D, E, Q, S, or T, Xaa2 is selected from A, H, I, S, T, or V, Xaa3 is selected from A, E, H, I, T, or V, Xaa4 is selected from A, D, E, or P, Xaa5 is selected from I, L, M, V, or Y, Xaa6 is selected from D, E, I, N, or Q, Xaa7 is selected from A, E, G, Q, or V, Xaa8 is selected from F, G, M, or W, Xaa9 is selected from I, P, T, or Y, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 19118-SEQ ID NO: 20117, wherein said at least one mutation drives increased lymph node tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 35. Disclosed herein are engineered AAV5 VP capsid polypeptides capable of forming an assembled virion that exhibits increased lymph node tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of high average flexibility (e.g., Xaa1 is selected from D, E, P, G, Q, S, or R); or wherein Xaa1 is selected from an amino acid of high hbond donors (e.g., Xaa1 is selected from R); or wherein Xaa1 is selected from an amino acid of high mol mass (e.g., Xaa1 is selected from Y, W, R, or F); or wherein Xaa2 is selected from an amino acid of low solubility (e.g., Xaa2 is selected from N or E); or wherein Xaa3 is selected from an amino acid of low average flexibility (e.g., Xaa3 is selected from W, M, or F); or wherein Xaa3 is selected from an amino acid of low mutability (e.g., Xaa3 is selected from R, H, K, P, Y, F, L, or C); or wherein Xaa4 is selected from an amino acid of low mutability (e.g., Xaa4 is selected from C); or wherein Xaa5 is selected from an amino acid of high mutability (e.g., Xaa5 is selected from N); or wherein Xaa5 is selected from an amino acid of medium mol mass (e.g., Xaa5 is selected from D, I, L, or N); or wherein Xaa6 is selected from an amino acid of high mol mass (e.g., Xaa6 is selected from Y, W, R, or F); or wherein Xaa6 is selected from an amino acid of high average flexibility (e.g., Xaa6 is selected from G or R); or wherein Xaa7 is selected from an amino acid of high average flexibility (e.g., Xaa7 is selected from D, E, K, P, I, N, Q, or S); or wherein Xaa7 is selected from an amino acid of low solubility (e.g., Xaa7 is selected from N, E); or wherein Xaa8 is selected from an amino acid of low solubility (e.g., Xaa8 is selected from N, E, or D); or wherein Xaa8 is selected from an amino acid of medium mutability (e.g., Xaa8 is selected from R or H); or wherein Xaa9 is selected from an amino acid of low mutability (e.g., Xaa9 is selected from P or K); or wherein Xaa9 is selected from an amino acid of high average flexibility (e.g., Xaa9 is selected from D, E, P, or S); or wherein Xaa9 is selected from an amino acid of high solubility (e.g., Xaa9 is selected from M or V); or any combination thereof.

In some embodiments, Xaa1 is selected from an amino acid of high average flexibility. In some embodiments, Xaa1 is selected from D, E, P, G, Q, S, or R. In some embodiments, Xaa1 is selected from an amino acid of high hbond donors. In some embodiments, Xaa1 is selected from R. In some embodiments, Xaa1 is selected from an amino acid of high mol mass. In some embodiments, Xaa1 is selected from Y, W, R, or F. In some embodiments, Xaa2 is selected from an amino acid of low solubility. In some embodiments, Xaa2 is selected from N or E. In some embodiments, Xaa3 is selected from an amino acid of low average flexibility. In some embodiments, Xaa3 is selected from W, M, or F. In some embodiments, Xaa3 is selected from an amino acid of low mutability. In some embodiments, Xaa3 is selected from R, H, K, P, Y, F, L, or C. In some embodiments, Xaa4 is selected from an amino acid of low mutability. In some embodiments, Xaa4 is selected from C. In some embodiments, Xaa5 is selected from an amino acid of high mutability. In some embodiments, Xaa5 is selected from N. In some embodiments, Xaa5 is selected from an amino acid of medium mol mass. In some embodiments, Xaa5 is selected from D, I, L, or N. In some embodiments, Xaa6 is selected from an amino acid of high mol mass. In some embodiments, Xaa6 is selected from Y, W, R, or F. In some embodiments, Xaa6 is selected from an amino acid of high average flexibility. In some embodiments, Xaa6 is selected from G, R. In some embodiments, Xaa7 is selected from an amino acid of high average flexibility. In some embodiments, Xaa7 is selected from D, E, K, P, I, N, Q, or S. In some embodiments, Xaa7 is selected from an amino acid of low solubility. In some embodiments, Xaa7 is selected from N or E. In some embodiments, Xaa8 is selected from an amino acid of low solubility. In some embodiments, Xaa8 is selected from N, E, or D. In some embodiments, Xaa8 is selected from an amino acid of medium mutability. In some embodiments, Xaa8 is selected from R or H. In some embodiments, Xaa9 is selected from an amino acid of low mutability. In some embodiments, Xaa9 is selected from P or K. In some embodiments, Xaa9 is selected from an amino acid of high average flexibility. In some embodiments, Xaa9 is selected from D, E, P, or S. In some embodiments, Xaa9 is selected from an amino acid of high solubility. In some embodiments, Xaa9 is selected from M or V.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 20118-SEQ ID NO: 21117, wherein said at least one mutation drives increased lymph node tissue tropism.

### C. Enriched Lymph Node Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 21118-SEQ ID NO: 22117, wherein said at least one mutation drives increased lymph node tissue tropism.

6.7.18. In Vivo Selected Mutated VP Polypeptides that Confer Increased Skin Tropism

The present disclosure provides AAV5 virions with a VP capsid polypeptide having at least one mutation in a region with residues that interact with target cells (e.g., a target skin cell in a target skin tissue of interest), where the at least one mutation confers increased skin tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased skin tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in skin over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), liver, skeletal muscle, heart, lung, spleen, lymph node, bone marrow, mammary gland, adrenal gland, thyroid, colon, sciatic nerve, and spinal cord tissues) was analyzed to identify a set of sequence rules for capsids that preferentially target skin tissue. Identification of positional frequency rules from in vivo data is described in detail in EXAMPLE 17.

Disclosed herein are engineered AAV5 VP capsid polypeptides capable of forming an assembled virion that exhibits increased skin tissue tropism as compared to wildtype AAV5 VP capsid polypeptide, wherein the engineered variant AAV5 VP1 capsid polypeptide sequence has one or more mutations, wherein Xaa1 is selected from A, C, K, Q, R, or T, or Xaa1 is selected from C, K, or R, or Xaa1 is C; or Xaa2 is selected from A, C, I, S, T, or V, or Xaa2 is selected from A, S, T, or V, or Xaa2 is V; or Xaa3 is selected from A, C, F, G, M, Q, S, or V, or Xaa3 is selected from A, C, F, M, or Q, or Xaa3 is C; or Xaa4 is selected from C, K, L, P, R, or W, or Xaa4 is selected from L, P, or R, or Xaa4 is R; or Xaa5 is selected from F, H, I, M, V, or Y, or Xaa5 is selected from M, V, or Y, or Xaa5 is Y; or Xaa6 is selected from F, H, I, M, N, Q, or S, or Xaa6 is selected from M, N, or Q, or Xaa6 is N; or Xaa7 is selected from A, H, K, M, N, R, or V, or Xaa7 is A, H, K, or R, or Xaa7 is K; or Xaa8 is selected from A, F, G, H, S, or Y, or Xaa8 is selected from A, F, or S, or Xaa8 is S; or Xaa9 is selected from A, E, G, P, Q, R, or S, or Xaa9 is selected from A, Q, or S, or Xaa9 is A.

Disclosed herein are engineered AAV5 VP capsid polypeptides capable of forming an assembled virion that exhibits increased skin tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from A, C, K, Q, R, or T. In some embodiments, Xaa1 is selected from C, K, or R. In some embodiments, Xaa1 is C. In some embodiments, Xaa2 is selected from A, C, I, S, T, or V. In some embodiments, Xaa2 is selected from A, S, T, or V. In some embodiments, Xaa2 is V. In some embodiments, Xaa3 is selected from A, C, F, G, M, Q, S, or V. In some embodiments, Xaa3 is selected from A, C, F, M, or Q. In some embodiments, Xaa3 is C. In some embodiments, Xaa4 is selected from C, K, L, P, R, or W. In some embodiments, Xaa4 is selected from L, P, or R. In some embodiments, Xaa4 is R. In some embodiments, Xaa5 is selected from F, H, I, M, V, or Y. In some embodiments, Xaa5 is selected from M, V, or Y. In some embodiments, Xaa5 is Y. In some embodiments, Xaa6 is selected from F, H, I, M, N, Q, or S. In some embodiments, Xaa6 is selected from M, N, or Q. In some embodiments, Xaa6 is N. In some embodiments, Xaa7 is selected from A, H, K, M, N, R, or V. In some embodiments, Xaa7 is A, H, K, or R. In some embodiments, Xaa7 is K. In some embodiments, Xaa8 is selected from A, F, G, H, S, or Y. In some embodiments, Xaa8 is selected from A, F, or S. In some embodiments, Xaa8 is S. In some embodiments, Xaa9 is selected from A, E, G, P, Q, R, or S. In some embodiments, Xaa9 is selected from A, Q, or S. In some embodiments, Xaa9 is A.

Disclosed herein are engineered AAV5 VP capsid polypeptides capable of forming an assembled virion that exhibits increased skin tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from A, C, K, Q, R, or T, Xaa2 is selected from A, C, I, S, T, or V, Xaa3 is selected from A, C, F, G, M, Q, S, or V, Xaa4 is selected from C, K, L, P, R, or W, Xaa5 is selected from F, H, I, M, V, or Y, Xaa6 is selected from F, H, I, M, N, Q, or S, Xaa7 is selected from A, H, K, M, N, R, or V, Xaa8 is selected from A, F, G, H, S, or Y, and Xaa9 is selected from A, E, G, P, Q, R, or S.

Disclosed herein are engineered AAV5 VP capsid polypeptides capable of forming an assembled virion that exhibits increased skin tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from A, C, K, Q, R, or T, Xaa2 is selected from A, C, I, S, T, or V, Xaa3 is selected from A, C, F, G, M, Q, S, or V, Xaa4 is selected from C, K, L, P, R, or W, Xaa5 is selected from F, H, I, M, V, or Y, Xaa6 is selected from F, H, I, M, N, Q, or S, Xaa7 is selected from A, H, K, M, N, R, or V, Xaa8 is selected from A, F, G, H, S, or Y, Xaa9 is selected from A, E, G, P, Q, R, or S, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 31991-SEQ ID NO: 32990, wherein said at least one mutation drives increased skin tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 40. Disclosed herein are engineered AAV5 VP capsid polypeptides capable of forming an assembled virion that exhibits increased skin tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of low surface accessibility (e.g., Xaa1 is selected from C); or wherein Xaa1 is selected from an amino acid of low volume (e.g., Xaa1 is selected from C); or wherein Xaa1 is selected from an amino acid of low mutability (e.g., Xaa1 is selected from C); or wherein Xaa2 is selected from an amino acid of high surface accessibility (e.g., Xaa2 is selected from R or K); or wherein Xaa2 is selected from an amino acid of high average flexibility (e.g., Xaa2 is selected from K, I, or N); or wherein Xaa2 is selected from an amino acid of low mutability (e.g., Xaa2 is selected from P or K); or wherein Xaa3 is selected from an amino acid of high hydropathy (e.g., Xaa3 is selected from I or V); or wherein Xaa4 is selected from an amino acid of low mutability (e.g., Xaa4 is selected from L, F, or Y); or wherein Xaa4 is selected from an amino acid of low average flexibility (e.g., Xaa4 is selected from W, H, F, or M); or wherein Xaa5 is selected from an amino acid of high average flexibility (e.g., Xaa5 is selected from G, R, K, I, or N); or wherein Xaa6 is selected from an amino acid of high average flexibility (e.g., Xaa6 is selected from G, R, K, I, or N); or wherein Xaa8 is selected from an amino acid of high surface accessibility (e.g., Xaa8 is selected from M, G, or F); or wherein Xaa8 is selected from an amino acid of low average flexibility (e.g., Xaa8 is selected from H, F, M, or W); or wherein Xaa8 is selected from an amino acid of low mutability (e.g., Xaa8 is selected from L, F, Y); or wherein Xaa9 is selected from an amino acid of high average flexibility (e.g., Xaa9 is selected from D, E, R, K, P, or G); or wherein Xaa9 is selected from an amino acid of high mutability (e.g., Xaa9 is selected from D, E, R, V, A, or H); or any combination thereof.

In some embodiments, Xaa1 is selected from an amino acid of low surface accessibility. In some embodiments, Xaa1 is selected from C. In some embodiments, Xaa1 is selected from an amino acid of low volume. In some embodiments, Xaa1 is selected from C. In some embodiments, Xaa1 is selected from an amino acid of low mutability. In some embodiments, Xaa1 is selected from C. In some embodiments, Xaa2 is selected from an amino acid of high surface accessibility. In some embodiments, Xaa2 is selected from R or K. In some embodiments, Xaa2 is selected from an amino acid of high average flexibility. In some embodiments, Xaa2 is selected from K, I, or N. In some embodiments, Xaa2 is selected from an amino acid of low mutability. In some embodiments, Xaa2 is selected from P or K. In some embodiments, Xaa3 is selected from an amino acid of high hydropathy. In some embodiments, Xaa3 is selected from I or V. In some embodiments, Xaa4 is selected from an amino acid of low mutability. In some embodiments, Xaa4 is selected from L, F, or Y. In some embodiments, Xaa4 is selected from an amino acid of low average flexibility. In some embodiments, Xaa4 is selected from W, H, F, or M. In some embodiments, Xaa5 is selected from an amino acid of high average flexibility. In some embodiments, Xaa5 is selected from G, R, K, I, or N. In some embodiments, Xaa6 is selected from an amino acid of high average flexibility. In some embodiments, Xaa6 is selected from G, R, K, I, or N. In some embodiments, Xaa8 is selected from an amino acid of high surface accessibility. In some embodiments, Xaa8 is selected from M, G, or F. In some embodiments, Xaa8 is selected from an amino acid of low average flexibility. In some embodiments, Xaa8 is selected from H, F, M, or W. In some embodiments, Xaa8 is selected from an amino acid of low mutability. In some embodiments, Xaa8 is selected from L, F, or Y. In some embodiments, Xaa9 is selected from an amino acid of high average flexibility. In some embodiments, Xaa9 is selected from D, E, R, K, P, or G. In some embodiments, Xaa9 is selected from an amino acid of high mutability. In some embodiments, Xaa9 is selected from D, E, R, V, A, or H.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 32991-SEQ ID NO: 33990, wherein said at least one mutation drives increased skin tissue tropism.

### C. Enriched Skin Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 33991-SEQ ID NO: 34990, wherein said at least one mutation drives increased skin tissue tropism.

6.7.19. In Vivo Selected Mutated VP Polypeptides that Confer Increased Bone Marrow Tropism

The present disclosure provides AAV5 virions with a VP capsid polypeptide having at least one mutation in a region with residues that interact with target cells (e.g., a target bone marrow cell in a target bone marrow tissue of interest), where the at least one mutation confers increased bone marrow tissue tropism as compared to a wildtype VP capsid polypeptide. In some embodiments, provided herein are AAV5 VP1 capsid polypeptide having a sequence homology of at least 80% to SEQ ID NO: 1, wherein the AAV5 VP1 capsid polypeptide has at least one mutation in a region from a position corresponding to 581 to a position corresponding to 589 of SEQ ID NO: 1 and wherein said at least one mutation drives increased bone marrow tropism. The following sequences rules and sequences also apply to the region in AAV5 VP2 (amino acid residues 445 to 453; VP2 sequence shown in SEQ ID NO: 1115) and AAV5 VP3 (amino acid residues 389 to 397; VP3 sequences shown in SEQ ID NO: 1116) corresponding to AAV5 VP1 amino acid residues 581 to 589. Thus, the present disclosure encompasses AAV5 VP2 capsid polypeptides and AAV5 VP3 capsid polypeptides having one or more mutations in the VP2 and VP3 regions corresponding to the AAV5 VP1 amino acid residues of the 581 to 589 region, where the one or more mutations comport to the rules or sequences in the following section.

### A. Positional Frequency Rules

In this section, unless otherwise specified, the frequency of a given amino acid residue occurring at a specified position corresponding to position 581 to position 589 of SEQ ID NO: 1 (generalized in SEQ ID NO: 2) in variants identified in bone marrow over the frequency of that given amino acid residue occurring at the specified position in variants identified in all other harvested tissues (CNS (cortex forebrain, cortex occipital, cortex temporal, thalamus, hypothalamus, substantia nigra, hippocampus DG, hippocampus CA1, hippocampus CA3, cerebellum), liver, skeletal muscle, heart, lung, spleen, lymph node, mammary gland, skin, adrenal gland, thyroid, colon, sciatic nerve, and spinal cord tissues) was analyzed to identify a set of sequence rules for capsids that preferentially target bone marrow tissue. Identification of positional frequency rules from in vivo data is described in detail in EXAMPLE 18.

Disclosed herein are engineered AAV5 VP capsid polypeptides capable of forming an assembled virion that exhibits increased bone marrow tissue tropism as compared to wildtype AAV5 VP capsid polypeptide, wherein the engineered variant AAV5 VP1 capsid polypeptide sequence has one or more mutations, wherein Xaa1 is selected from A, E, G, Q, S, or T, or Xaa1 is selected from A, E, or T, or Xaa1 is E; or Xaa2 is selected from A, I, Q, S, T, V, or Y, or Xaa2 is selected from A, S, T, or Xaa2 is A; or Xaa3 is selected from A, G, I, M, Q, S, or T, or Xaa3 is selected from A, Q, or T, or Xaa3 is Q; or Xaa4 is selected from A, E, P, Q, T, or V, or Xaa4 is selected from A, P, or Q, or Xaa4 is Q; or Xaa5 is selected from F, I, L, M, Q, V, or Y, or Xaa5 is selected from F, V, or Y, or Xaa5 is V; or Xaa6 is selected from F, I, N, Q, S, or V, or Xaa6 is selected from I, N, Q, or S, or Xaa6 is S; or Xaa7 is selected from A, C, M, S, or V, or Xaa7 is A, C, or V, or Xaa7 is C; or Xaa8 is selected from A, C, D, G, M, S, or Y, or Xaa8 is selected from A, M, S, or Y, or Xaa8 is M; or Xaa9 is selected from D, E, G, L, P, S, or Y, or Xaa9 is selected from D, E, or P, or Xaa9 is P.

Disclosed herein are engineered AAV5 VP capsid polypeptides capable of forming an assembled virion that exhibits increased bone marrow tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules. In some embodiments, Xaa1 is selected from A, E, G, Q, S, or T. In some embodiments, Xaa1 is selected from A, E, or T. In some embodiments, Xaa1 is E. In some embodiments, Xaa2 is selected from A, I, Q, S, T, V, or Y. In some embodiments, Xaa2 is selected from A, S, T. In some embodiments, Xaa2 is A. In some embodiments, Xaa3 is selected from A, G, I, M, Q, S, or T. In some embodiments, Xaa3 is selected from A, Q, or T. In some embodiments, Xaa3 is Q. In some embodiments, Xaa4 is selected from A, E, P, Q, T, or V. In some embodiments, Xaa4 is selected from A, P, or Q. In some embodiments, Xaa4 is Q. In some embodiments, Xaa5 is selected from F, I, L, M, Q, V, or Y. In some embodiments, Xaa5 is selected from F, V, or Y. In some embodiments, Xaa5 is V. In some embodiments, Xaa6 is selected from F, I, N, Q, S, or V. In some embodiments, Xaa6 is selected from I, N, Q, or S. In some embodiments, Xaa6 is S. In some embodiments, Xaa7 is selected from A, C, M, S, or V. In some embodiments, Xaa7 is A, C, or V. In some embodiments, Xaa7 is C. In some embodiments, Xaa8 is selected from A, C, D, G, M, S, or Y. In some embodiments, Xaa8 is selected from A, M, S, or Y. In some embodiments, Xaa8 is M. In some embodiments, Xaa9 is selected from D, E, G, L, P, S, or Y. In some embodiments, Xaa9 is selected from D, E, or P. In some embodiments, Xaa9 is P.

Disclosed herein are engineered AAV5 VP capsid polypeptides capable of forming an assembled virion that exhibits increased bone marrow tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are selected from the following rules: Xaa1 is selected from A, E, G, Q, S, or T, Xaa2 is selected from A, I, Q, S, T, V, or Y, Xaa3 is selected from A, G, I, M, Q, S, or T, Xaa4 is selected from A, E, P, Q, T, or V, Xaa5 is selected from F, I, L, M, Q, V, or Y, Xaa6 is selected from F, I, N, Q, S, or V, Xaa7 is selected from A, C, M, S, or V, Xaa8 is selected from A, C, D, G, M, S, or Y, and Xaa9 is selected from D, E, G, L, P, S, or Y.

Disclosed herein are engineered AAV5 VP capsid polypeptides capable of forming an assembled virion that exhibits increased bone marrow tissue tropism as compared to wildtype AAV5 VP capsid polypeptide, wherein the engineered variant AAV5 VP capsid polypeptide sequence has one or more mutations, wherein the VP1 polypeptide sequence has said one or more mutations in a region from a position corresponding to 581 in SEQ ID NO: 2 to a position corresponding to 589 in SEQ ID NO: 2 and wherein said one or more mutations are as follows: Xaa1 is selected from A, E, G, Q, S, or T, Xaa2 is selected from A, I, Q, S, T, V, or Y, Xaa3 is selected from A, G, I, M, Q, S, or T, Xaa4 is selected from A, E, P, Q, T, or V, Xaa5 is selected from F, I, L, M, Q, V, or Y, Xaa6 is selected from F, I, N, Q, S, or V, Xaa7 is selected from A, C, M, S, or V, Xaa8 is selected from A, C, D, G, M, S, or Y, Xaa9 is selected from D, E, G, L, P, S, or Y, or any combination thereof.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 4118-SEQ ID NO: 5117, wherein said at least one mutation drives increased bone marrow tissue tropism.

### B. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 32. Disclosed herein are engineered AAV5 VP capsid polypeptides capable of forming an assembled virion that exhibits increased bone marrow tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of high hydropathy (e.g., Xaa1 is selected from V, I, or L); or wherein Xaa1 is selected from an amino acid of low mutability (e.g., Xaa1 is selected from Y, L, F, or C); or wherein Xaa2 is selected from an amino acid of low hydropathy (e.g., Xaa2 is selected from Y or W); or wherein Xaa2 is selected from an amino acid of high mol mass (e.g., Xaa2 is selected from W); or wherein Xaa2 is selected from an amino acid of low surface accessibility (e.g., Xaa2 is selected from W or A); or wherein Xaa2 is selected from an amino acid of low hydrophilicity (e.g., Xaa2 is selected from W); or wherein Xaa2 is selected from an amino acid of low mutability (e.g., Xaa2 is selected from C); or wherein Xaa2 is selected from an amino acid of low average flexibility (e.g., Xaa2 is selected from W, M, or F); or wherein Xaa5 is selected from an amino acid of low average flexibility (e.g., Xaa5 is selected from W, M, or F); or wherein Xaa6 is selected from an amino acid of low average flexibility (e.g., Xaa6 is selected from W, M, or F); or wherein Xaa6 is selected from an amino acid of low mutability (e.g., Xaa6 is selected from Y, F, L, or C); or wherein Xaa6 is selected from an amino acid of high solubility (e.g., Xaa6 is selected from W, F, I, or L); or wherein Xaa7 is selected from an amino acid of low surface accessibility (e.g., Xaa7 is selected from C); or wherein Xaa7 is selected from an amino acid of high surface accessibility (e.g., Xaa7 is selected from D or N); or wherein Xaa7 is selected from an amino acid of low mutability (e.g., Xaa7 is selected from C); or wherein Xaa7 is selected from an amino acid of high solubility (e.g., Xaa7 is selected from C); or wherein Xaa7 is selected from an amino acid of low solubility (e.g., Xaa7 is selected from D); or wherein Xaa8 is selected from an amino acid of low charge (e.g., Xaa8 is selected from D or E); or wherein Xaa8 is selected from an amino acid of high mutability (e.g., Xaa8 is selected from D, E, A, or T); or wherein Xaa9 is selected from an amino acid of high mol mass (e.g., Xaa9 is selected from H or F); or wherein Xaa9 is selected from an amino acid of low mutability (e.g., Xaa9 is selected from Y, F, or L); or any combination thereof.

In some embodiments, Xaa1 is selected from an amino acid of high hydropathy. In some embodiments, Xaa1 is selected from V, I, or L. In some embodiments, Xaa1 is selected from an amino acid of low mutability. In some embodiments, Xaa1 is selected from Y, L, F, or C. In some embodiments, Xaa2 is selected from an amino acid of low hydropathy. In some embodiments, Xaa2 is selected from Y or W. In some embodiments, Xaa2 is selected from an amino acid of high mol mass. In some embodiments, Xaa2 is selected from W. In some embodiments, Xaa2 is selected from an amino acid of low surface accessibility. In some embodiments, Xaa2 is selected from W or A. In some embodiments, Xaa2 is selected from an amino acid of low hydrophilicity. In some embodiments, Xaa2 is selected from W. In some embodiments, Xaa2 is selected from an amino acid of low mutability. In some embodiments, Xaa2 is selected from C. In some embodiments, Xaa2 is selected from an amino acid of low average flexibility. In some embodiments, Xaa2 is selected from W, M, or F. In some embodiments, Xaa5 is selected from an amino acid of low average flexibility. In some embodiments, Xaa5 is selected from W, M, or F. In some embodiments, Xaa6 is selected from an amino acid of low average flexibility. In some embodiments, Xaa6 is selected from W, M, or F. In some embodiments, Xaa6 is selected from an amino acid of low mutability. In some embodiments, Xaa6 is selected from Y, F, L, or C. In some embodiments, Xaa6 is selected from an amino acid of high solubility. In some embodiments, Xaa6 is selected from W, F, I, or L. In some embodiments, Xaa7 is selected from an amino acid of low surface accessibility. In some embodiments, Xaa7 is selected from C. In some embodiments, Xaa7 is selected from an amino acid of high surface accessibility. In some embodiments, Xaa7 is selected from D or N. In some embodiments, Xaa7 is selected from an amino acid of low mutability. In some embodiments, Xaa7 is selected from C. In some embodiments, Xaa7 is selected from an amino acid of high solubility. In some embodiments, Xaa7 is selected from C. In some embodiments, Xaa7 is selected from an amino acid of low solubility. In some embodiments, Xaa7 is selected from D. In some embodiments, Xaa8 is selected from an amino acid of low charge. In some embodiments, Xaa8 is selected from D or E. In some embodiments, Xaa8 is selected from an amino acid of high mutability. In some embodiments, Xaa8 is selected from D, E, A, or T. In some embodiments, Xaa9 is selected from an amino acid of high mol mass. In some embodiments, Xaa9 is selected from H or F. In some embodiments, Xaa9 is selected from an amino acid of low mutability. In some embodiments, Xaa9 is selected from Y, F, or L.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 5118-SEQ ID NO: 6117, wherein said at least one mutation drives increased bone marrow tissue tropism.

### C. Enriched Bone Marrow Sequences

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 6118-SEQ ID NO: 7117, wherein said at least one mutation drives increased bone marrow tissue tropism.

6.7.20. In Vivo Selected Mutated VP Polypeptides that Confer Increased Skeletal Muscle Tropism or Cardiac Muscle Tropism

### A. ML Rules

For the following set of rules described in this paragraph, favored biophysical properties and favored amino acid residues at each position in the 581 to 589 region of an engineered AAV5 VP1 capsid polypeptide, were determined using in vivo data and two ML models, which are described in EXAMPLE 22. Disclosed herein are engineered AAV5 VP capsid polypeptides capable of forming an assembled virion that exhibits increased skeletal muscle tissue tropism or cardiac muscle tissue tropism as compared to an rAAV virion having a wildtype AAV5 VP capsid polypeptide, wherein the engineered AAV5 VP capsid polypeptide sequence has one or more mutations, wherein the engineered AAV5 VP1 capsid polypeptide sequence has said one or more mutations in a region from a position corresponding to 581 in SEQ ID NO: 2 to a position corresponding to 589 in SEQ ID NO: 2 and wherein Xaa1 is selected from an amino acid of low solubility (e.g., Xaa1 is selected from D, E, R, K, P, N, or Q); or wherein Xaa1 is selected from an amino acid of low hydropathy (e.g., Xaa1 is selected from D, E, R, K, Q, N, Y, or P); or wherein Xaa1 is selected from an amino acid of high surface accessibility (e.g., Xaa1 is selected from E, R, or K); or wherein Xaa2 is selected from an amino acid of high hydropathy (e.g., Xaa2 is selected from V, I, F, L, or C); or wherein Xaa2 is selected from an amino acid of low mutability (e.g., Xaa2 is selected from R, V, I, H, or C); or wherein Xaa2 is selected from an amino acid of medium volume (e.g., Xaa2 is selected from E, V, or Q); or wherein Xaa3 is selected from an amino acid of low solubility (e.g., Xaa3 is selected from D, R, or Q); or wherein Xaa4 is selected from an amino acid of low solubility (e.g., Xaa4 is selected from D, E, P, or N); or wherein Xaa4 is selected from an amino acid of low charge (e.g., Xaa4 is selected from D or E); or wherein Xaa5 is selected from an amino acid of low amino acid solubility (e.g., Xaa5 is selected from D, E, R, K, N, or Q); or wherein Xaa8 is selected from an amino acid of low solubility (e.g., Xaa8 is selected from D, E, K, P, or N); or wherein Xaa8 is selected from an amino acid of high flexibility index (e.g., Xaa8 is selected from Q, S, P, E, or D); or wherein Xaa8 is selected from an amino acid of high surface accessibility (e.g., Xaa8 is selected from S, D, P, N, E, R, or K); or any combination thereof.

In some embodiments, Xaa1 is selected from an amino acid of low solubility. In some embodiments, Xaa1 is selected from D, E, R, K, P, N, or Q. In some embodiments, Xaa1 is selected from an amino acid of low hydropathy. In some embodiments, Xaa1 is selected from D, E, R, K, Q, N, Y, or P. In some embodiments, Xaa1 is selected from an amino acid of high surface accessibility. In some embodiments, Xaa1 is selected from E, R, or K. In some embodiments, Xaa2 is selected from an amino acid of high hydropathy. In some embodiments, Xaa2 is selected from V, I, F, L, or C. In some embodiments, Xaa2 is selected from an amino acid of low mutability. In some embodiments, Xaa2 is selected from R, V, I, H, or C. In some embodiments, Xaa2 is selected from an amino acid of medium volume. In some embodiments, Xaa2 is selected from E, V, or Q. In some embodiments, Xaa3 is selected from an amino acid of low solubility. In some embodiments, Xaa3 is selected from D, R, or Q. In some embodiments, Xaa4 is selected from an amino acid of low solubility. In some embodiments, Xaa4 is selected from D, E, P, or N. In some embodiments, Xaa4 is selected from an amino acid of low charge. In some embodiments, Xaa4 is selected from D or E. In some embodiments, Xaa5 is selected from an amino acid of low amino acid solubility. In some embodiments, Xaa5 is selected from D, E, R, K, N, or Q. In some embodiments, Xaa8 is selected from an amino acid of low solubility. In some embodiments, Xaa8 is selected from D, E, K, P, or N. In some embodiments, Xaa8 is selected from an amino acid of high flexibility index. In some embodiments, Xaa8 is selected from Q, S, P, E, or D. In some embodiments, Xaa8 is selected from an amino acid of high surface accessibility. In some embodiments, Xaa8 is selected from S, D, P, N, E, R, or K.

In some embodiments, provided herein are AAV5 VP capsid polypeptide having at least one mutation in a region from a position corresponding to 581 to a position corresponding to 589 of AAV5 VP1 and having at least 70%, at least 75%, at least 80%, at least 85%, at least 90%, at least 92%, at least 95%, at least 97%, at least 99%, or 100% sequence identity to any sequence selected from SEQ ID NO: 25118-SEQ ID NO: 26117, wherein said at least one mutation drives increased skeletal muscle tissue tropism or cardiac muscle tissue tropism.

### 1.1. NUMBERED EMBODIMENTS

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

In a further aspect, the following embodiments are provided. All numerical references to a preceding embodiment refer to the embodiment so numbered within the same subsection. In yet a further aspect, rAAV comprising the recombinant or engineered VP capsid polypeptides of the following numbered embodiments are provided, as are methods of using pharmaceutical compositions comprising the rAAV for treatment of a subject in need thereof.

### Series A Embodiments

In the Series A embodiments, “recombinant” adeno-associated (AAV) VP1 capsid polypeptide is synonymous with “engineered” adeno-associated (AAV) VP1 capsid polypeptide.
1. A recombinant adeno-associated virus (AAV) VP1 capsid polypeptide having the amino acid sequence of SEQ ID NO:2, wherein Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V; and wherein the polypeptide does not have the sequence of any of SEQ ID NO: 1, SEQ ID NO:3, SEQ ID NO:4, SEQ ID NO:5, SEQ ID NO:6, SEQ ID NO:7, and SEQ ID NO: 8. 2. A recombinant adeno-associated virus (AAV) VP1 capsid polypeptide having at least one mutation in a residue corresponding to residue 581 to residue 589 in SEQ ID NO: 1, wherein the mutation confers tissue tropism for a first tissue as compared to a second tissue and wherein the AAV VP1 capsid polypeptide does not have the sequence of any of SEQ ID NO:1, SEQ ID NO: 3, SEQ ID NO:4, SEQ ID NO:5, SEQ ID NO:6, SEQ ID NO:7, and SEQ ID NO:8. 3. The recombinant AAV VP1 capsid polypeptide of embodiment 2, wherein the AAV VP1 capsid polypeptide is an AAV5 VP1 capsid polypeptide. 4. The recombinant AAV VP1 capsid polypeptide of any one of embodiments 1-3, wherein a specific order of the residues at residue 581-589 corresponding to SEQ ID NO: 1 results in a specific tissue tropism. 5. The recombinant AAV VP1 capsid polypeptide of any one of embodiments 1-4, wherein the first tissue is selected from adipose, adrenal gland, aorta, brain (including hippocampus: dentate gyrus, CA1 and CA3; cerebellum, caudate, putamen, midbrain, pons, hypothalamus, cortex-including occipital, temporal and forebrain; substantia nigra, and thalamus), bone marrow, cecum, colon, dorsal root ganglion, duodenum, epididymis, esophagus, cyc, gallbladder, heart, ileum, jejunum, kidney, lung, lymph nodes, mammary gland, ovary, pancreas, parathyroid gland, peripheral nerve, pituitary, prostate, salivary gland, seminal vesicle, skeletal muscle, skin, spinal cord, spleen, stomach, testis, thymus, thyroid, trachea, urinary bladder, uterus, and vagina; wherein the second tissue is selected from: adipose, adrenal gland, aorta, brain (including hippocampus: dentate gyrus, CA1 and CA3; cerebellum, caudate, putamen, midbrain, pons, hypothalamus, cortex-including occipital, temporal and forebrain; substantia nigra, and thalamus), bone marrow, cecum, colon, dorsal root ganglion, duodenum, epididymis, esophagus, eye, gallbladder, heart, ileum, jejunum, kidney, lung, lymph nodes, mammary gland, ovary, pancreas, parathyroid gland, peripheral nerve, pituitary, prostate, salivary gland, seminal vesicle, skeletal muscle, skin, spinal cord, spleen, stomach, testis, thymus, thyroid, trachea, urinary bladder, uterus, and vagina, and wherein the first tissue and the second tissue are different. 6. The recombinant AAV VP1 capsid polypeptide of any one of embodiments 2-4, wherein the rAAV has increased ability to infect a tissue selected from adipose, adrenal gland, aorta, brain (including hippocampus: dentate gyrus, CA1 and CA3; cerebellum, caudate, putamen, midbrain, pons, hypothalamus, cortex-including occipital, temporal and forebrain; substantia nigra, and thalamus), bone marrow, cecum, colon, dorsal root ganglion, duodenum, epididymis, esophagus, eye, gallbladder, heart, ilcum, jejunum, kidney, lung, lymph nodes, mammary gland, ovary, pancreas, parathyroid gland, peripheral nerve, pituitary, prostate, salivary gland, seminal vesicle, skeletal muscle, skin, spinal cord, spleen, stomach, testis, thymus, thyroid, trachea, urinary bladder, uterus, and vagina. Following intravenous administration as compared to a rAAV having a VP1 capsid polypeptide of SEQ ID NO: 1. 7. The recombinant AAV VP1 capsid polypeptide of any one of embodiments 2-6, wherein the rAAV exhibits from about a 1.0005-fold to about a 1000-fold increased accumulation in the first tissue as compared to the second tissue. 8. The recombinant AAV VP1 capsid polypeptide of any one of embodiments 2-6, wherein the rAAV exhibits at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the first tissue as compared to the second tissue. 9. The recombinant capsid polypeptide of any preceding embodiment, further comprising one or more mutations at an amino acid residue outside of the 581-589 region, wherein the one or more mutations at an amino acid residue outside of the 581-589 region confers improved manufacturability, improved viral assembly, improved tissue targeting/tropism, or any combination thereof. 10. The recombinant AAV VP1 capsid polypeptide of any of embodiments 1-9, wherein Xaa1 is selected from A, G, K, M, N, Q, R, S, or T. 11. The recombinant capsid polypeptide of any preceding embodiment, wherein Xaa1 is selected from A, K, M, or T. 12. The recombinant capsid polypeptide of any preceding embodiment, wherein Xaa1 is K. 13. The recombinant capsid polypeptide of any preceding embodiment, wherein Xaa2 is selected from A, C, H, I, K, S, T, or V. 14. The recombinant capsid polypeptide of any preceding embodiment, wherein Xaa2 is selected from A, S, T, or V. 15. The recombinant capsid polypeptide of any preceding embodiment, wherein Xaa2 is T. 16. The recombinant capsid polypeptide of any preceding embodiment, wherein Xaa3 is selected from A, G, H, K, M, N, Q, R, S, T, or V. 17. The recombinant capsid polypeptide of any preceding embodiment, wherein Xaa3 is selected from A, M, or T. 18. The recombinant capsid polypeptide of any preceding embodiment, wherein Xaa3 comprises A or T. 19. The recombinant capsid polypeptide of any preceding embodiment, wherein Xaa4 is selected from L, M, P, Q, R, T, or W. 20. The recombinant capsid polypeptide of any preceding embodiment, wherein Xaa4 is selected from L, P, Q, or T. 21. The recombinant capsid polypeptide of any preceding embodiment, wherein Xaa4 is P; 22. The recombinant capsid polypeptide of any preceding embodiment, wherein Xaa5 is selected from F, H, I, K, M, T, or Y. 23. The recombinant capsid polypeptide of any preceding embodiment, wherein Xaa5 is selected from H, I, or Y. 24. The recombinant capsid polypeptide of any preceding embodiment, wherein Xaa5 is Y. 25. The recombinant capsid polypeptide of any preceding embodiment, wherein Xaa6 is selected from E, G, H, L, M, N, Q, T, or W. 26. The recombinant capsid polypeptide of any preceding embodiment, wherein Xaa6 is selected from N, or Q. 27. The recombinant capsid polypeptide of any preceding embodiment, wherein Xaa6 is N. 28. The recombinant capsid polypeptide of any preceding embodiment, wherein Xaa7 is selected from A, C, G, H, L, M, R or S. 29. The recombinant capsid polypeptide of any preceding embodiment, wherein Xaa7 is selected from A, C, H or M. 30. The recombinant capsid polypeptide of any preceding embodiment, wherein Xaa7 is A. 31. The recombinant capsid polypeptide of any preceding embodiment, wherein Xaa8 is selected from A, C, D, F, G, H, M, Q, S, V, W, or Y. 32. The recombinant capsid polypeptide of any preceding embodiment, wherein Xaa8 is selected from G, M, Q, or S. 33. The recombinant capsid polypeptide of any preceding embodiment, wherein Xaa8 is G. 34. The recombinant capsid polypeptide of any preceding embodiment, wherein Xaa9 is selected from A, C, E, G, H, M, N, P, Q, S, V, or W. 35. The recombinant capsid polypeptide of any preceding embodiment, wherein Xaa9 is selected from E, G, or P. 36. The recombinant capsid polypeptide of any preceding embodiment, wherein Xaa9 is G. 37. The recombinant capsid polypeptide of any of embodiments 1-9, wherein Xaa1 is selected from A, D, E, G, L, M, N, Q, S, T, or V. 38. The recombinant capsid polypeptide of embodiment 37, wherein Xaa1 is selected from A, D, E, M, or T. 39. The recombinant capsid polypeptide of embodiment 37, wherein Xaa1 is E. 40. The recombinant capsid polypeptide of any of embodiments 37-39, wherein Xaa2 is selected from A, C, D, E, G, H, I, N, P, Q, S, T, or V. 41. The recombinant capsid polypeptide of any of embodiments 37-40, wherein Xaa2 is selected from A, S, T, or V. 42. The recombinant capsid polypeptide of any of embodiments 37-41, wherein Xaa2 is A. 43. The recombinant capsid polypeptide of any of embodiments 37-42, wherein Xaa3 is selected from A, D, E, G, H, M, N, Q, S, T, or V. 44. The recombinant capsid polypeptide of any of embodiments 37-43, wherein Xaa3 is selected from D, E, N, Q or T. 45 The recombinant capsid polypeptide of any of embodiments 37-44, wherein Xaa3 is D or T. 46. The recombinant capsid polypeptide of any of embodiments 37-45, wherein Xaa4 is selected from A, D, E, G, H, N, P, Q, S, or T. 47. The recombinant capsid polypeptide of any of embodiments 37-46, wherein Xaa4 is selected from D, E, P, or Q. 48. The recombinant capsid polypeptide of any of embodiments 37-47, wherein Xaa4 is E. 49. The recombinant capsid polypeptide of any of embodiments 37-48, wherein Xaa5 is selected from A, C, D, E, G, H, N, Q, S, T, or Y. 50. The recombinant capsid polypeptide of any of embodiments 37-49, wherein Xaa5 is selected from D, E, N, Q or T. 51. The recombinant capsid polypeptide of any of embodiments 37-50, wherein Xaa5 is N. 52. The recombinant capsid polypeptide of any of embodiments 37-51, wherein Xaa6 is selected from A, D, E, G, H, N, P, Q, S, or T. 53. The recombinant capsid polypeptide of any of embodiments 37-52, wherein Xaa6 is selected from D, N, or Q. 54. The recombinant capsid polypeptide of any of embodiments 37-53, wherein Xaa6 is D. 55. The recombinant capsid polypeptide of any of embodiments 37-54, wherein Xaa7 is selected from A, C, D, E, G, H, N, Q, S, or T. 56. The recombinant capsid polypeptide of any of embodiments 37-55, wherein Xaa7 is selected from A, D, E or G. 57. The recombinant capsid polypeptide of any of embodiments 37-56, wherein Xaa7 is A. 58. The recombinant capsid polypeptide of any of embodiments 37-57, wherein Xaa8 is selected from A, C, D, E, G, H, N, Q, S, or T. 59. The recombinant capsid polypeptide of any of embodiments 37-58, wherein Xaa8 comprises A, D, G, or S. 60. The recombinant capsid polypeptide of any of embodiments 37-59, wherein Xaa8 is G. 61. The recombinant capsid polypeptide of any of embodiments 37-60, wherein Xaa9 is selected from A, D, E, G, H, N, P, Q, S, or T. 62. The recombinant capsid polypeptide of any of embodiments 37-61, wherein Xaa9 is selected from A, D, G, or P. 63. The recombinant capsid polypeptide of any of embodiments 37-62, wherein Xaa9 is G. 64. A recombinant capsid polypeptide of any of embodiments 1-36 combined with the recombinant capsid polypeptide of any of embodiments 37-63, wherein the VP1 capsid is capable of forming an assembled virion that exhibits increased tropism for liver tissue when compared to a virion that comprises the AAV5 VP1 capsid polypeptide of SEQ ID NO:1. 65. The recombinant capsid polypeptide of embodiment 1, wherein Xaa1 is not K, and wherein the VP1 capsid is capable of forming an assembled virion that exhibits decreased tropism for liver tissue when compared to a virion that comprises the AAV5 VP1 capsid of SEQ ID NO:1. 66. The recombinant capsid polypeptide of embodiment 1, wherein Xaa1 is not A, K, M, or T, and wherein the VP1 capsid is capable of forming an assembled virion that exhibits decreased tropism for liver tissue when compared to a virion that comprises the AAV5 VP1 capsid of SEQ ID NO:1. 67. The recombinant capsid polypeptide of embodiment 1, wherein Xaa1 is not A, G, K, M, N, Q, R, S, or T, and wherein the VP1 capsid is capable of forming an assembled virion that exhibits decreased tropism for liver tissue when compared to a virion that comprises the AAV5 VP1 capsid of SEQ ID NO:1. 68. The recombinant capsid polypeptide of embodiment 1, or any of embodiments 65-67, wherein Xaa2 is not T, and wherein the VP1 capsid is capable of forming an assembled virion that exhibits decreased tropism for liver tissue when compared to a virion that comprises the AAV5 VP1 capsid of SEQ ID NO:1. 69. The recombinant capsid polypeptide of embodiment 1, or any of embodiments 65-68, wherein Xaa2 is not A, S, T, or V, and wherein the VP1 capsid is capable of forming an assembled virion that exhibits decreased tropism for liver tissue when compared to a virion that comprises the AAV5 VP1 capsid of SEQ ID NO:1. 70. The recombinant capsid polypeptide of embodiment 1, or any of embodiments 65-69, wherein Xaa2 is not A, C, H, I, K, S, T, or V, and wherein the VP1 capsid is capable of forming an assembled virion that exhibits decreased tropism for liver tissue when compared to a virion that comprises the AAV5 VP1 capsid of SEQ ID NO:1. 71. The recombinant capsid polypeptide of embodiment 1, or any of embodiments 65-70, wherein Xaa3 is not A or T, and wherein the VP1 capsid is capable of forming an assembled virion that exhibits decreased tropism for liver tissue when compared to a virion that comprises the AAV5 VP1 capsid of SEQ ID NO: 1. 72. The recombinant capsid polypeptide of embodiment 1, or any of embodiments 65-71, Xaa3 is not A, M, or T, and wherein the VP1 capsid is capable of forming an assembled virion that exhibits decreased tropism for liver tissue when compared to a virion that comprises the AAV5 VP1 capsid of SEQ ID NO:1. 73. The recombinant capsid polypeptide of embodiment 1, or any of embodiments 65-72, wherein Xaa3 is not A, G, H, K, M, N, Q, R, S, T, or V, and wherein the VP1 capsid is capable of forming an assembled virion that exhibits decreased tropism for liver tissue when compared to a virion that comprises the AAV5 VP1 capsid of SEQ ID NO: 1. 74. The recombinant capsid polypeptide of embodiment 1, or any of embodiments 65-73, wherein Xaa4 is not P, and wherein the VP1 capsid is capable of forming an assembled virion that exhibits decreased tropism for liver tissue when compared to a virion that comprises the AAV5 VP1 capsid of SEQ ID NO:1. 75. The recombinant capsid polypeptide of embodiment 1, or any of embodiments 65-74, wherein Xaa4 is not L, P, Q, or T, and wherein the VP1 capsid is capable of forming an assembled virion that exhibits decreased tropism for liver tissue when compared to a virion that comprises the AAV5 VP1 capsid of SEQ ID NO:1. 76. The recombinant capsid polypeptide of embodiment 1, or any of embodiments 65-75, wherein Xaa4 is not L, M, P, Q, R, T, or W, and wherein the VP1 capsid is capable of forming an assembled virion that exhibits decreased tropism for liver tissue when compared to a virion that comprises the AAV5 VP1 capsid of SEQ ID NO:1. 77. The recombinant capsid polypeptide of embodiment 1, or any of embodiments 65-76, wherein Xaa5 is not Y, and wherein the VP1 capsid is capable of forming an assembled virion that exhibits decreased tropism for liver tissue when compared to a virion that comprises the AAV5 VP1 capsid of SEQ ID NO:1. 78. The recombinant capsid polypeptide of embodiment 1, or any of embodiments 65-77, wherein Xaa5 is not H, I, or Y, and wherein the VP1 capsid is capable of forming an assembled virion that exhibits decreased tropism for liver tissue when compared to a virion that comprises the AAV5 VP1 capsid of SEQ ID NO:1. 79. The recombinant capsid polypeptide of embodiment 1, or any of embodiments 65-78, wherein Xaa5 is not F, H, I, K, M, T, or Y, and wherein the VP1 capsid is capable of forming an assembled virion that exhibits decreased tropism for liver tissue when compared to a virion that comprises the AAV5 VP1 capsid of SEQ ID NO:1. 80. The recombinant capsid polypeptide of embodiment 1, or any of embodiments 65-79, wherein Xaa6 is not N, and wherein the VP1 capsid is capable of forming an assembled virion that exhibits decreased tropism for liver tissue when compared to a virion that comprises the AAV5 VP1 capsid of SEQ ID NO:1. 81. The recombinant capsid polypeptide of embodiment 1, or any of embodiments 65-80, wherein Xaa6 is not N, or Q, and wherein the VP1 capsid is capable of forming an assembled virion that exhibits decreased tropism for liver tissue when compared to a virion that comprises the AAV5 VP1 capsid of SEQ ID NO:1. 82. The recombinant capsid polypeptide of embodiment 1, or any of embodiments 65-81, wherein Xaa6 is not E, G, H, L, M, N, Q, T, or W, and wherein the VP1 capsid is capable of forming an assembled virion that exhibits decreased tropism for liver tissue when compared to a virion that comprises the AAV5 VP1 capsid of SEQ ID NO:1. 83. The recombinant capsid polypeptide of embodiment 1, or any of embodiments 65-82, wherein Xaa7 is not A, and wherein the VP1 capsid is capable of forming an assembled virion that exhibits decreased tropism for liver tissue when compared to a virion that comprises the AAV5 VP1 capsid of SEQ ID NO:1. 84. The recombinant capsid polypeptide of embodiment 1, or any of embodiments 65-83, wherein Xaa7 is not A, C, H or M, and wherein the VP1 capsid is capable of forming an assembled virion that exhibits decreased tropism for liver tissue when compared to a virion that comprises the AAV5 VP1 capsid of SEQ ID NO:1. 85. The recombinant capsid polypeptide of embodiment 1, or any of embodiments 65-84, wherein Xaa7 is not A, C, G, H, L, M, R or S, and wherein the VP1 capsid is capable of forming an assembled virion that exhibits decreased tropism for liver tissue when compared to a virion that comprises the AAV5 VP1 capsid of SEQ ID NO:1. 86. The recombinant capsid polypeptide of embodiment 1, or any of embodiments 65-85, wherein Xaa8 is not G, and wherein the VP1 capsid is capable of forming an assembled virion that exhibits decreased tropism for liver tissue when compared to a virion that comprises the AAV5 VP1 capsid of SEQ ID NO:1. 87. The recombinant capsid polypeptide of embodiment 1, or any of embodiments 65-86, wherein Xaa8 is not G, M, Q, or S, and wherein the VP1 capsid is capable of forming an assembled virion that exhibits decreased tropism for liver tissue when compared to a virion that comprises the AAV5 VP1 capsid of SEQ ID NO:1. 88. The recombinant capsid polypeptide of embodiment 1, or any of embodiments 65-87, wherein Xaa8 is not A, C, D, F, G, H, M, Q, S, V, W, or Y, and wherein the VP1 capsid is capable of forming an assembled virion that exhibits decreased tropism for liver tissue when compared to a virion that comprises the AAV5 VP1 capsid of SEQ ID NO: 1. 89. The recombinant capsid polypeptide of embodiment 1, or any of embodiments 65-88, Xaa9 is not G, and wherein the VP1 capsid is capable of forming an assembled virion that exhibits decreased tropism for liver tissue when compared to a virion that comprises the AAV5 VP1 capsid of SEQ ID NO: 1. 90. The recombinant capsid polypeptide of embodiment 1, or any of embodiments 65-89, wherein Xaa9 is not E, G, or P, and wherein the VP1 capsid is capable of forming an assembled virion that exhibits decreased tropism for liver tissue when compared to a virion that comprises the AAV5 VP1 capsid of SEQ ID NO:1. 91. The recombinant capsid polypeptide of embodiment 1, or any of embodiments 65-90, wherein Xaa9 is not A, C, E, G, H, M, N, P, Q, S, V, or W, and wherein the VP1 capsid is capable of forming an assembled virion that exhibits decreased tropism for liver tissue when compared to a virion that comprises the AAV5 VP1 capsid of SEQ ID NO: 1. 92. A recombinant capsid polypeptide of any of embodiments 65-91 combined with the recombinant capsid polypeptide of any of embodiments 37-63, wherein the VP1 capsid is capable of forming an assembled virion that exhibits decreased tropism for liver tissue when compared to a virion that comprises the AAV5 VP1 capsid polypeptide of SEQ ID NO: 1. 93. A recombinant adeno-associated virus (AAV) VP1 capsid polypeptide having at least one residue corresponding to residue 581 to residue 589 in SEQ ID NO: 2, wherein the at least one residue is: Xaa1 and Xaa1 is selected from A, G, K, M, N, Q, R, S, or T; Xaa2 and Xaa2 is selected from A, C, H, I, K, S, T, or V; Xaa3 and Xaa3 is selected from A, G, H, K, M, N, Q, R, S, T, or V; Xaa4 and Xaa4 is selected from L, M, P, Q, R, T, or W; Xaa5 and Xaa5 is selected from F, H, I, K, M, T, or Y; Xaa6 and Xaa6 is selected from E, G, H, L, M, N, Q, T, or W; Xaa7 and Xaa7 is selected from A, C, G, H, L, M, R or S; Xaa8 and Xaa8 is selected from A, C, D, F, G, H, M, Q, S, V, W, or Y; Xaa9 and Xaa9 is selected from A, C, E, G, H, M, N, P, Q, S, V, or W; or any combination thereof, wherein the AAV VP1 capsid polypeptide is capable of exhibiting tissue tropism for liver tissue. 94. A recombinant adeno-associated virus AAV VP1 capsid polypeptide having at least one mutation in a residue of region 581 to residue 589 in SEQ ID NO: 1, wherein the mutation confers at least about a two-fold increased accumulation in a non-liver tissue as compared to a liver tissue, as compared to AAV5 VP1, and wherein the AAV VP1 capsid polypeptide does not have the sequence of any of SEQ ID NO:1, SEQ ID NO:3, SEQ ID NO:4, SEQ ID NO:5, SEQ ID NO:6, SEQ ID NO:7, and SEQ ID NO:8. 95. The recombinant AAVVP1 capsid polypeptide of embodiment 94, wherein the mutation confers at least about a three-fold, at least about a four-fold, at least about a five-fold, at least about a ten-fold, at least about a twenty-fold, at least about a fifty-fold, at least about a 75-fold, at least about a 100-fold increased accumulation in a non-liver tissue as compared to a liver tissue. 96. The recombinant AAVVP1 capsid polypeptide of embodiment 94, wherein the mutation confers from about a 1.0005-fold to about a 1000-fold increased accumulation in a non-liver tissue as compared to a liver tissue. 97. The recombinant capsid polypeptide of any preceding embodiment, further comprising one or more mutations at an amino acid residue outside of the 581-589 region, with reference to SEQ ID NO: 1, wherein the resulting recombinant capsid is capable of forming an assembled virion that exhibits desired tissue targeting/tropism. 98. The recombinant capsid polypeptide of embodiment 97, wherein the one or more mutations at an amino acid residue outside of the 581-589 region confers improved manufacturability, improved viral assembly, improved tissue targeting/tropism, or any combination thereof. 99. A vector capable of replication in prokaryotic cells, wherein the vector comprises a polynucleotide encoding the recombinant capsid polypeptide of any preceding embodiment. 100. The vector of embodiment 99, wherein the vector is a plasmid. 101. A library comprising a plurality of plasmids of embodiment 100, the plurality of plasmids comprising a plurality of different AAV VP1-encoding polynucleotides. 102. The plasmid library of embodiment 101, wherein the library encodes at least 1×109 different AAV VP1 capsid polypeptides. 103. The plasmid library of embodiment 102, wherein the library encodes at least 5×109 different AAV VP1 capsid polypeptides. 104. The plasmid library of embodiment 103, wherein the library encodes at least 1×1010 different AAV VP1 capsid polypeptides. 105. The plasmid library of embodiment 104, wherein the library encodes at least 5×1010 different AAV VP1 capsid polypeptides. 106. The plasmid library of embodiment 105, wherein the library encodes at least 7.5×1010 different AAV VP1 capsid polypeptides. 107. The plasmid library of embodiment 106, wherein the library encodes at least 1×1011 different AAV VP1 capsid polypeptides. 108. The plasmid library of embodiment 107, wherein the library encodes at least 2.5×1011 different AAV VP1 capsid polypeptides. 109. The plasmid library of embodiment 108, wherein the library encodes at least 5×1011 different AAV VP1 capsid polypeptides. 110. A prokaryotic cell comprising the vector of embodiment 100. 111. The prokaryotic cell of embodiment 110, wherein prokaryotic cell is an E. coli cell and the vector is a plasmid. 112. A library comprising a plurality of E. coli cells of embodiment 111, wherein the plurality of cells comprises a plurality of plasmids, wherein the plurality of plasmids comprises a plurality of different AAV VP1-encoding polynucleotides. 113. A library comprising a plurality of polypeptides of any of embodiments 1-98, the plurality having different primary amino acid sequences. 114. The library of embodiment 113, wherein the library comprises at least from about 1×105 to at least about 5×1011 different AAV VP1 capsid polypeptides. 115. A recombinant AAV virion (rAAV), the virion comprising an AAV VP1 capsid polypeptide of any of embodiments 1-98. 116. The rAAV virion of embodiment 115, wherein the rAAV has reduced tropism for human liver as compared to a rAAV having a VP1 capsid polypeptide having the sequence of SEQ ID NO:1. 117. The rAAV virion of embodiment 115 or embodiment 116, wherein the rAAV has increased ability to cross the blood-brain barrier following intravenous administration as compared to a rAAV having a VP1 capsid polypeptide having the sequence of SEQ ID NO:1. 118. The rAAV virion of any one of embodiments 115-117, wherein the rAAV has increased ability to infect one or more brain regions selected from hippocampus, dentate gyrus, cerebral cortex, temporal cortex, occipital cortex, thalamus, forebrain, substantia nigra, hypothalamus, and cerebellum, following intravenous, intrathecal, intracerebral ventricular, or intracisternal magna administration as compared to a rAAV having a VP1 capsid polypeptide having the sequence of SEQ ID NO: 1. 119. The rAAV virion of any one of embodiments 115-118, wherein the rAAV has increased ability to infect human retinal cells following intravitreal injection as compared to a rAAV having a VP1 capsid polypeptide having the sequence of SEQ ID NO: 1. 120. The rAAV virion of any one of embodiments 115-119, wherein the rAAV has increased ability to infect human skeletal muscle following intravenous administration as compared to a rAAV having a VP1 capsid polypeptide having the sequence of SEQ ID NO:1. 121. The rAAV virion of any one of embodiments 115, and 117-120, wherein the rAAV has increased tropism for human liver as compared to a rAAV having a VP1 capsid polypeptide having the sequence of SEQ ID NO: 1. 122. The rAAV virion of embodiment 115, 116, or 121, wherein the rAAV has increased ability to infect a tissue selected from adipose, adrenal gland, aorta, brain (including hippocampus: dentate gyrus, CA1 and CA3; cerebellum, caudate, putamen, midbrain, pons, hypothalamus, cortex-including occipital, temporal and forebrain; substantia nigra, and thalamus), bone marrow, cecum, colon, dorsal root ganglion, duodenum, epididymis, esophagus, eye, gallbladder, heart, ileum, jejunum, kidney, lung, lymph nodes, mammary gland, ovary, pancreas, parathyroid gland, peripheral nerve, pituitary, prostate, salivary gland, seminal vesicle, skeletal muscle, skin, spinal cord, spleen, stomach, testis, thymus, thyroid, trachea, urinary bladder, uterus, and vagina, following intravenous administration as compared to a rAAV having a VP1 capsid polypeptide of SEQ ID NO:1. 123. The rAAV virion of any one of embodiments 115-122, wherein the virion further comprises a vector genome, the vector genome comprising a therapeutic polynucleotide encoding any of the following: a therapeutic RNA selected from a guide RNA or a tRNA, or transgene encoding a protein under control of regulatory sequences that direct transgene expression in infected human cells. 124. The rAAV virion of embodiment 123, wherein the transgene encodes a protein selected from the transgene products of Table 1. 125. A library comprising a plurality of rAAV virions of any one of embodiments 115-124, wherein the plurality of rAAV virions comprise a plurality of VP1 capsid polypeptides with different primary amino acid sequences. 126. The library of embodiment 125, wherein the library comprises at least about 1×10⁵ to at least about 5×10¹¹ different AAV VP1 capsid polypeptides different AAV VP1 capsid polypeptides. 127. A pharmaceutical composition comprising the rAAV of embodiment 123 or embodiment 124 and a pharmaceutically acceptable carrier. 128. A method of treatment, comprising: administering an effective amount of the pharmaceutical composition of embodiment 127 to a patient in need thereof. 129. The method of embodiment 128, wherein the effective amount of the rAAV is less than the effective amount of a wild type rAAV. 130. The method of embodiment 128, wherein the effective amount of the rAAV is less than the effective amount of an otherwise comparable rAAV lacking one or more than one mutation at a position corresponding to residue 581 to residue 589 of SEQ ID NO: 1. 131. The method of any one of embodiments 128-130, wherein the effective amount of the results in lower toxicity in the patient as compared to the effective amount of the wild type rAAV, the otherwise comparable rAAV, or both. 132. The method of embodiment 128, wherein the effective amount is at least from 1×10⁵ viral genomes/kg patient weight to 5×10¹⁴ viral genomes/kg. 133. The method of any one of embodiments 128-132, wherein the rAAV is administered intravenously. 134. The method of any one of embodiments 128-132, wherein the rAAV is administered intrathecally. 135. The method of any one of embodiments 128-132, wherein the rAAV is administered by intracisternal magna administration. 136. The method of any one of embodiments 128-132, wherein the rAAV is administered by intravitreal injection. 137. A method of identifying an AAV VP1 capsid polypeptide that confers tropism for a desired tissue, comprising: administering an aliquot of the library of any one of embodiments 101-109, or 112-144, or 125-126 to a non-human primate; and identifying the sequences of AAV capsid sequence of rAAV that had infected the desired tissue. 138. The method of embodiment 137, wherein the library aliquot is administered intravenously. 139. The method of embodiment 137 wherein the library aliquot is administered intrathecally. 140. The method of embodiment 137, wherein the library aliquot is administered by intra-cisterna magna administration. 141. The method of embodiment 137, wherein the library aliquot is administered by intracerebral ventricular injection. 142. A method of formulating the therapeutic polynucleotide of any one of embodiments 123-124 in a virion, the method comprising: transfecting a cell with plasmid encoding for the recombinant capsid polypeptide of any one of embodiments 1-98 and transfecting the cell with a plasmid encoding for the therapeutic polynucleotide, wherein upon transfection, the cell produces the virion within which is packaged the therapeutic polynucleotide. 143. A composition comprising an AAV virion comprising the recombinant capsid polypeptide of any one of embodiments 1-98 within which is packaged the therapeutic polynucleotide of any one of embodiments 123-124. 144. A recombinant AAV VP1 capsid polypeptide having any one of the VP1 capsid mutations recited in Table 8 (SEQ ID NO:115-1114), and wherein the VP1 capsid polypeptide does not have the sequence of any of SEQ ID NO:1, SEQ ID NO:3, SEQ ID NO:4, SEQ ID NO:5, SEQ ID NO:6, SEQ ID NO:7, and SEQ ID NO:8. 145. A recombinant AAV VP1 capsid polypeptide having any of the VP1 capsid mutations recited in Table 8 (SEQ ID NO:115-1114), wherein the mutation confers tissue tropism for a first tissue as compared to a second tissue and wherein the AAV VP1 capsid polypeptide does not have the sequence of any of SEQ ID NO: 1, SEQ ID NO:3, SEQ ID NO: 4, SEQ ID NO:5, SEQ ID NO:6, SEQ ID NO:7, and SEQ ID NO:8. 146. The recombinant AAV VP1 capsid polypeptide of embodiment 145, wherein the mutation confers at least about a two-fold increased accumulation of rAAV comprising the mutated VP1 protein in a non-liver tissue as compared to a liver tissue as compared to accumulation of rAAV comprising AAV5 VP1 (SEQ ID NO:1), wherein the mutated rAAV and AAV5 rAAV are each administered intravenously at the same titer, and wherein the VP1 capsid polypeptide does not have the sequence of any of SEQ ID NO:1, SEQ ID NO:3, SEQ ID NO:4, SEQ ID NO:5, SEQ ID NO:6, SEQ ID NO:7, and SEQ ID NO:8. 147. A composition comprising an AAV virion comprising the recombinant capsid polypeptide of any one of embodiments 144-146, within which is packaged a therapeutic polynucleotide encoding any of the following: a therapeutic RNA selected from a guide RNA or a tRNA, or transgene encoding a protein under control of regulatory sequences that direct transgene expression in infected human cells.

### Series B Embodiments—CNS Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a central nervous system (CNS) tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid.
3. An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a central nervous system (CNS) tissue as compared to an rAAV virion having a wildtype AAV5 VP capsid polypeptide of SEQ ID NO: 1. 5. The engineered AAV VP capsid polypeptide of any one of embodiments 1-2, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 7118-SEQ ID NO: 10117. 6. The engineered AAV VP capsid polypeptide of embodiment 4, wherein the region from the residue corresponding to residue 581 to the residue corresponding to residue 589, inclusive, has a sequence of any one of SEQ ID NO: 7118-SEQ ID NO: 10117. 7. The engineered AAV VP capsid polypeptide of embodiment 3, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 7118-SEQ ID NO: 10117. 8. The engineered AAV VP capsid polypeptide of embodiment 6, wherein the Xaa1 to Xaa9 region of SEQ ID NO: 2 has a sequence of any one of SEQ ID NO: 7118-SEQ ID NO: 10117.9. The engineered AAV VP capsid polypeptide of any one of embodiments 2-3, wherein:
   Xaa1 is selected from the group consisting of A, C, K, M, Q, R, T, and W; or
   Xaa2 is selected from the group consisting of F, I, K, R, T, and W; or
   Xaa3 is selected from the group consisting of A, H, N, R, and W; or
   Xaa4 is selected from the group consisting of E, G, I, M, Q, and R; or
   Xaa5 is selected from the group consisting of C, G, K, I, M, and R; or
   Xaa6 is selected from the group consisting of I, K, L, P, Q, R, and Y; or
   Xaa7 is selected from the group consisting of D, I, K, R, V, and W; or
   Xaa8 is selected from the group consisting of C, G, H, K, L, and V; or
   Xaa9 is selected from the group consisting of I, K, L, R, and V; or
   any combination thereof.
10. The engineered AAV VP capsid polypeptide of embodiment 5, wherein Xaa1 is selected from K, Q, R, or W. 11. The engineered AAV VP capsid polypeptide of embodiment 5, wherein Xaa2 is selected from F, I, R or T. 12. The engineered AAV VP capsid polypeptide of embodiment 5, wherein Xaa3 is selected from A, R, or W. 13. The engineered AAV VP capsid polypeptide of embodiment 5, wherein Xaa4 is selected from E, M, or R. 14. The engineered AAV VP capsid polypeptide of embodiment 5, wherein Xaa5 is selected from K, I, or R. 15.

The engineered AAV VP capsid polypeptide of embodiment 5, wherein Xaa6 is selected from K, R, or Y. 16. The engineered AAV VP capsid polypeptide of embodiment 5, wherein Xaa7 is selected from I, R, or V. 17. The engineered AAV VP capsid polypeptide of embodiment 5, wherein Xaa8 is selected from H, K, or V. 18. The engineered AAV VP capsid polypeptide of embodiment 5, wherein Xaa9 is selected from I, K, or R. 19. The engineered AAV VP capsid polypeptide of embodiment 5, wherein Xaa1 is K. 20. The engineered AAV VP capsid polypeptide of embodiment 5, wherein Xaa2 is R. 21. The engineered AAV VP capsid polypeptide of embodiment 5, wherein Xaa3 is R. 22. The engineered AAV VP capsid polypeptide of embodiment 5, wherein Xaa4 is R. 23. The engineered AAV VP capsid polypeptide of embodiment 5, wherein Xaa5 is I. 24. The engineered AAV VP capsid polypeptide of embodiment 5, wherein Xaa6 is R. 25. The engineered AAV VP capsid polypeptide of embodiment 5, wherein Xaa7 is V. 26. The engineered AAV VP capsid polypeptide of embodiment 5, wherein Xaa8 is H. 27. The engineered AAV VP capsid polypeptide of embodiment 5, wherein Xaa9 is R. 28. The engineered AAV VP capsid polypeptide of any one of embodiments 1-8, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, at least 98%, or at least 99% identical to any one of SEQ ID NO: 7118-SEQ ID NO: 8117. 29. The engineered AAV VP capsid polypeptide of embodiment 27, wherein the region from residue 581 to residue 589 of SEQ ID NO: 1 has a sequence of any one of SEQ ID NO: 7118-SEQ ID NO: 8117. 30. The engineered AAV VP capsid polypeptide of any one of embodiments 2-3, wherein Xaa1 has low amino acid solubility. 31. The engineered AAV VP capsid polypeptide of embodiment 25, wherein Xaa1 is selected from K, R, or Q. 32. The engineered AAV VP capsid polypeptide of any one of embodiments 2-3, wherein Xaa1 has low amino acid hydropathy. 33. The engineered AAV VP capsid polypeptide of embodiment 27, wherein Xaa1 is selected from K or R. 34. The engineered AAV VP capsid polypeptide of any one of embodiments 2-3, wherein Xaa1 has a high average amino acid flexibility index. 35. The engineered AAV VP capsid polypeptide of embodiment 29, wherein Xaa1 is selected from D, E, R, K, G, I, N, Q, or S. 36. The engineered AAV VP capsid polypeptide of any one of embodiments 2-3, wherein Xaa1 has high hydrogen bond donors. 37. The engineered AAV VP capsid polypeptide of embodiment 31, wherein Xaa1 is selected from K, R. 38. The engineered AAV VP capsid polypeptide of any one of embodiments 2-3, wherein Xaa1 has low amino acid mutability. 39. The engineered AAV VP capsid polypeptide of embodiment 33, wherein Xaa1 is selected from K, R, P, or H. 40. The engineered AAV VP capsid polypeptide of any one of embodiments 2-3, wherein Xaa2 has low amino acid solubility. 41. The engineered AAV VP capsid polypeptide of embodiment 35, wherein Xaa2 is selected from R, K, Q, or S. 42. The engineered AAV VP capsid polypeptide of any one of embodiments 2-3, wherein Xaa2 has low amino acid hydropathy. 43. The engineered AAV VP capsid polypeptide of embodiment 37, wherein Xaa2 is selected from R, K, D, E, N, Q, H, P, Y, W, S, or T. 44. The engineered AAV VP capsid polypeptide of any one of embodiments 2-3, wherein Xaa2 has high amino acid charge. 45. The engineered AAV VP capsid polypeptide of embodiment 39, wherein Xaa2 is selected from R, K, or H. 46. The engineered AAV VP capsid polypeptide of any one of embodiments 2-3, wherein Xaa3 has high amino acid solubility. 47. The engineered AAV VP capsid polypeptide of embodiment 41, wherein Xaa3 is selected from A, M, V, W, L, or I. 48. The engineered AAV VP capsid polypeptide of any one of embodiments 2-3, wherein Xaa5 has high amino acid solubility. 49. The engineered AAV VP capsid polypeptide of embodiment 43, wherein Xaa5 is selected from C, M, V, W, L, or I. 50. The engineered AAV VP capsid polypeptide of any one of embodiments 2-3, wherein Xaa5 has high hydropathy. 51. The engineered AAV VP capsid polypeptide of embodiment 45, wherein Xaa5 is selected from M, V, or I. 52. The engineered AAV VP capsid polypeptide of any one of embodiments 2-3, wherein Xaa5 has low average amino acid flexibility index. 53. The engineered AAV VP capsid polypeptide of embodiment 47, wherein Xaa5 is selected from M, W, F, or C. 54. The engineered AAV VP capsid polypeptide of any one of embodiments 2-3, wherein Xaa8 has high amino acid solubility. 55. The engineered AAV VP capsid polypeptide of embodiment 49, wherein Xaa8 is selected from H, V, or I. 56. The engineered AAV VP capsid polypeptide of any one of embodiments 29-54, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 8118-SEQ ID NO: 9117. 57. The engineered AAV VP capsid polypeptide of any one of embodiments 25-50, wherein the region from the residue corresponding to residue 581 to the residue corresponding to residue 589 inclusive has a sequence of any one of SEQ ID NO: 8118-SEQ ID NO: 9117. 58. The engineered AAV VP capsid polypeptide of any one of embodiments 1-56, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof. 59. The engineered AAV VP capsid polypeptide of any one of embodiments 1-57, wherein tropism for CNS tissue is measured as a relative accumulation of the rAAV virion in a CNS tissue as compared to a non-CNS tissue, wherein the non-CNS tissue consists collectively of liver, skeletal muscle, heart, lung, spleen, lymph node, bone marrow, mammary gland, skin, adrenal gland, thyroid, colon, sciatic nerve, and spinal cord. 60. The engineered AAV VP capsid polypeptide of any one of embodiments 1-58, wherein the higher tissue tropism is a 1.0005-fold to about a 1000-fold increased accumulation in the CNS tissue as compared to a non-CNS tissue. 61. The engineered AAV VP capsid polypeptide of embodiment 59, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the CNS tissue as compared to a non-CNS tissue.

### Series C Embodiments—Liver De-Targeted Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a non-liver tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid. 3. An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a non-liver tissue as compared to an rAAV virion having a wildtype AAV5 VP capsid polypeptide of SEQ ID NO: 1. 5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, at least 98%, or at least 99% identical to any one of SEQ ID NO: 46438-SEQ ID NO: 47437. 6. The engineered AAV VP capsid polypeptide of embodiment 5, wherein the region from the residue corresponding to residue 581 to the residue corresponding to residue 589, inclusive, has a sequence of any one of SEQ ID NO: 46438-SEQ ID NO: 47437. 7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 46438-SEQ ID NO: 47437. 8. The engineered AAV VP capsid polypeptide of embodiment 7, wherein the Xaa1 to Xaa9 region of SEQ ID NO: 2 has a sequence of any one of SEQ ID NO: 46438-SEQ ID NO: 47437. 9. The engineered AAV VP capsid polypeptide of any one of embodiments 2-4, wherein:
   Xaa1 excludes A, G, K, M, N, Q, R, S, or T; or
   Xaa2 excludes A, C, I, K, S, T, or V; or
   Xaa3 excludes A, G, I, K, M, Q, R, S, T, or V; or
   Xaa4 excludes A, I, K, L, P, Q, R, S, T, or V; or
   Xaa5 excludes F, I, L, M, T, V, or Y; or
   Xaa6 excludes F, H, M, N, Q, S, or Y; or
   Xaa7 excludes A, C, K, M, Q or S; or
   Xaa8 excludes A, C, F, G, M, Q, or S; or
   Xaa9 excludes E, F, L, Q, R, or Y; or
   any combination thereof.
10. The engineered AAV VP capsid polypeptide of embodiment 9, wherein Xaa1 excludes A, K, Q, or R. 11. The engineered AAV VP capsid polypeptide of embodiment 9, wherein Xaa2 excludes A, K, S, or T. 12. The engineered AAV VP capsid polypeptide of embodiment 9, wherein Xaa3 excludes A, K, Q, S, or T. 13. The engineered AAV VP capsid polypeptide of embodiment 9, wherein Xaa4 excludes K, I, S, or V. 14. The engineered AAV VP capsid polypeptide of embodiment 9, wherein Xaa5 excludes F, L, or Y. 15. The engineered AAV VP capsid polypeptide of embodiment 9, wherein Xaa6 excludes M or N. 16. The engineered AAV VP capsid polypeptide of embodiment 9, wherein Xaa7 excludes A, C, or S. 17. The engineered AAV VP capsid polypeptide of embodiment 9, wherein Xaa8 excludes A, C, M, or S. 18. The engineered AAV VP capsid polypeptide of embodiment 9, wherein Xaa9 excludes L, Q, or R. 19. The engineered AAV VP capsid polypeptide of embodiment 9, wherein Xaa1 excludes K. 20. The engineered AAV VP capsid polypeptide of embodiment 9, wherein Xaa2 excludes A. 21. The engineered AAV VP capsid polypeptide of embodiment 9, wherein Xaa3 excludes K, Q, or T. 22. The engineered AAV VP capsid polypeptide of embodiment 9, wherein Xaa3 excludes K. 23. The engineered AAV VP capsid polypeptide of embodiment 9, wherein Xaa4 excludes K. 24. The engineered AAV VP capsid polypeptide of embodiment 9, wherein Xaa5 excludes F. 25. The engineered AAV VP capsid polypeptide of embodiment 9, wherein Xaa6 excludes N. 26. The engineered AAV VP capsid polypeptide of embodiment 9, wherein Xaa7 excludes S. 27. The engineered AAV VP capsid polypeptide of embodiment 9, wherein Xaa8 excludes C. 28. The engineered AAV VP capsid polypeptide of embodiment 9, wherein Xaa9 excludes R. 29. The engineered AAV VP capsid polypeptide of any one of embodiments 2-4, wherein Xaa1 has low solubility. 30. The engineered AAV VP capsid polypeptide of embodiment 29, wherein Xaa1 is selected from D and P. 31. The engineered AAV VP capsid polypeptide of any one of embodiments 2-4, wherein Xaa1 has low mutability. 32. The engineered AAV VP capsid polypeptide of embodiment 31, wherein Xaa1 is selected from Xaa1 is selected from C, K, and L. 33. The engineered AAV VP capsid polypeptide of any one of embodiments 2-4, wherein Xaa2 has low solubility. 34. The engineered AAV VP capsid polypeptide of embodiment 33, wherein Xaa2 is selected from Xaa2 is selected from N, K, P, E, and D. 35. The engineered AAV VP capsid polypeptide of any one of embodiments 2-4, wherein Xaa2 has low hydropathy. 36. The engineered AAV VP capsid polypeptide of embodiment 35, wherein Xaa2 is selected from Xaa2 is selected from D, E, R, K, H, N, and Q. 37. The engineered AAV VP capsid polypeptide of any one of embodiments 2-4, wherein Xaa2 has low charge. 38. The engineered AAV VP capsid polypeptide of embodiment 37, wherein Xaa2 is selected from D and E. 39. The engineered AAV VP capsid polypeptide of any one of embodiments 2-4, wherein Xaa2 has high number of total potential hydrogen bonds. 40. The engineered AAV VP capsid polypeptide of embodiment 39, wherein Xaa2 is selected from H, N, Q, D, E, and R. 41. The engineered AAV VP capsid polypeptide of any one of embodiments 2-4, wherein Xaa2 has medium volume. 42. The engineered AAV VP capsid polypeptide of embodiment 41, wherein Xaa2 is selected from D, E, V, P, N, and T. 43. The engineered AAV VP capsid polypeptide of any one of embodiments 2-4, wherein Xaa3 has low solubility. 44. The engineered AAV VP capsid polypeptide of embodiment 43, wherein Xaa3 is selected from P and D. 45. The engineered AAV VP capsid polypeptide of any one of embodiments 2-4, wherein Xaa4 has medium volume. 46. The engineered AAV VP capsid polypeptide of embodiment 45, wherein Xaa4 is selected from D, E, V, P, N, and T. 47. The engineered AAV VP capsid polypeptide of any one of embodiments 2-4, wherein Xaa5 has low solubility. 48. The engineered AAV VP capsid polypeptide of embodiment 47, wherein Xaa5 is selected from N, P, E, and D. 49. The engineered AAV VP capsid polypeptide of any one of embodiments 2-4, wherein Xaa8 has low solubility. 50. The engineered AAV VP capsid polypeptide of embodiment 49, wherein Xaa8 is selected from K and Q. 51. The engineered AAV VP capsid polypeptide of any one of embodiments 2-4, wherein Xaa8 has low hydropathy. 52. The engineered AAV VP capsid polypeptide of embodiment 51, wherein Xaa8 is selected from K and R. 53. The engineered AAV VP capsid polypeptide of any one of embodiments 2-4, wherein Xaa8 has high surface accessibility. 54. The engineered AAV VP capsid polypeptide of embodiment 53, wherein Xaa8 is selected from E, R, and K. 55. The engineered AAV VP capsid polypeptide of any one of embodiments 1-54, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 46438-SEQ ID NO: 47437. 56. The engineered AAV VP capsid polypeptide of embodiment 55, wherein the region from the residue corresponding to residue 581 to the residue corresponding to residue 589 inclusive has a sequence of any one of SEQ ID NO: 46438-SEQ ID NO: 47437. 57. The engineered AAV VP capsid polypeptide of any one of embodiments 1-56, wherein tropism for a non-liver tissue is measured as a relative accumulation of the rAAV virion in a non-liver tissue as compared to a liver tissue, wherein the non-liver tissue consists collectively of CNS tissue, skeletal muscle, heart, lung, spleen, lymph node, bone marrow, mammary gland, skin, adrenal gland, thyroid, colon, sciatic nerve, and spinal cord. 58. The engineered AAV VP capsid polypeptide of embodiment 57, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof. 59. The engineered AAV VP capsid polypeptide of any one of embodiments 57-58, wherein the higher tissue tropism is a 1.0005-fold to about a 1000-fold increased accumulation in the non-liver tissue as compared to a liver tissue. 60. The engineered AAV VP capsid polypeptide of embodiment 59, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the non-liver tissue as compared to a liver tissue.

### Series D Embodiments—Liver Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a liver tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid.
3. An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a liver tissue as compared to an rAAV virion having a wildtype AAV5 VP capsid polypeptide of SEQ ID NO: 1. 5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 43438-SEQ ID NO: 46437. 6. The engineered AAV VP capsid polypeptide of embodiment 5, wherein the region from the residue corresponding to residue 581 to the residue corresponding to residue 589, inclusive, has a sequence of any one of SEQ ID NO: 43438-SEQ ID NO: 46437. 7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 43438-SEQ ID NO: 46437. 8. The engineered AAV VP capsid polypeptide of embodiment 7, wherein the Xaa1 to Xaa9 region of SEQ ID NO: 2 has a sequence of any one of SEQ ID NO: 43438-SEQ ID NO: 46437. 9. The engineered AAV VP capsid polypeptide of any one of embodiments 2-4, wherein:
   Xaa1 is selected from the group consisting of A, G, K, M, N, Q, R, S, and T; or
   Xaa2 is selected from the group consisting of A, C, I, K, S, T, and V; or
   Xaa3 is selected from the group consisting of A, G, I, K, M, Q, R, S, T, and V; or
   Xaa4 is selected from the group consisting of A, I, K, L, P, Q, R, S, T, and V; or
   Xaa5 is selected from the group consisting of F, I, L, M, T, V, and Y; or
   Xaa6 is selected from the group consisting of F, H, M, N, Q, S, and Y; or
   Xaa7 is selected from the group consisting of A, C, K, M, Q and S; or
   Xaa8 is selected from the group consisting of A, C, F, G, M, Q, and S; or
   Xaa9 is selected from the group consisting of E, F, L, Q, R, and Y; or
any combination thereof.
10. The engineered AAV VP capsid polypeptide of embodiment 9, wherein Xaa1 is selected from A, K, Q, and R. 11. The engineered AAV VP capsid polypeptide of embodiment 9, wherein Xaa1 is K. 12. The engineered AAV VP capsid polypeptide of embodiment 9, wherein Xaa2 is selected from A, K, S, and T. 13. The engineered AAV VP capsid polypeptide of embodiment 9, wherein Xaa2 is A. 14. The engineered AAV VP capsid polypeptide of embodiment 9, wherein Xaa3 is selected from A, K, Q, S, and T. 15. The engineered AAV VP capsid polypeptide of embodiment 9, wherein Xaa3 is selected from K, Q, and T. 16. The engineered AAV VP capsid polypeptide of embodiment 9, wherein Xaa3 is K. 17. The engineered AAV VP capsid polypeptide of embodiment 9, wherein Xaa4 is selected from K, I, S, and V. 18. The engineered AAV VP capsid polypeptide of embodiment 9, wherein Xaa4 is K. 19. The engineered AAV VP capsid polypeptide of embodiment 9, wherein Xaa5 is selected from F, L, and Y. 20. The engineered AAV VP capsid polypeptide of embodiment 9, wherein Xaa5 is F. 21. The engineered AAV VP capsid polypeptide of embodiment 9, wherein Xaa6 is selected from M and N. 22.

The engineered AAV VP capsid polypeptide of embodiment 9, wherein Xaa6 is N. 23. The engineered AAV VP capsid polypeptide of embodiment 9, wherein Xaa7 is selected from A, C, and S. 24. The engineered AAV VP capsid polypeptide of embodiment 9, wherein Xaa7 is S. 25. The engineered AAV VP capsid polypeptide of embodiment 9, wherein Xaa8 is selected from A, C, M, and S. 26. The engineered AAV VP capsid polypeptide of embodiment 9, wherein Xaa8 is C. 27. The engineered AAV VP capsid polypeptide of embodiment 9, wherein Xaa9 is selected from L, Q, and R. 28. The engineered AAV VP capsid polypeptide of embodiment 9, wherein Xaa9 is R. 29. The engineered AAV VP capsid polypeptide of any one of embodiments 1-28, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 43438-SEQ ID NO: 44437. 30. The engineered AAV VP capsid polypeptide of embodiment 29, wherein the region from residue 581 to residue 589 of SEQ ID NO: 1 has a sequence of any one of SEQ ID NO: 43438-SEQ ID NO: 44437.
31. The engineered AAV VP capsid polypeptide of any one of embodiments 2-4, wherein Xaa1 has high surface accessibility. 32. The engineered AAV VP capsid polypeptide of embodiment 31, wherein Xaa1 is selected from K, R, and E. 33. The engineered AAV VP capsid polypeptide of any one of embodiments 2-4, wherein Xaa1 has Low hydropathy (<−3.5). 34. The engineered AAV VP capsid polypeptide of embodiment 33, wherein Xaa1 is selected from K and R. 35. The engineered AAV VP capsid polypeptide of any one of embodiments 2-4, wherein Xaa1 has Low amino acid mutability. 36. The engineered AAV VP capsid polypeptide of embodiment 35, wherein Xaa1 is selected from H, P, K, and R. 37. The engineered AAV VP capsid polypeptide of any one of embodiments 2-4, wherein Xaa1 has Low amino acid solubility. 38. The engineered AAV VP capsid polypeptide of embodiment 37, wherein Xaa1 is selected from Q, K, and R. 39. The engineered AAV VP capsid polypeptide of any one of embodiments 2-4, wherein Xaa2 has High surface accessibility.
40. The engineered AAV VP capsid polypeptide of embodiment 39, wherein Xaa2 is selected from E, R, and K. 41. The engineered AAV VP capsid polypeptide of any one of embodiments 2-4, wherein Xaa2 has Low hydropathy. 42. The engineered AAV VP capsid polypeptide of embodiment 41, wherein Xaa2 is selected from K and R. 43. The engineered AAV VP capsid polypeptide of any one of embodiments 2-4, wherein Xaa2 has High amino acid volume. 44. The engineered AAV VP capsid polypeptide of embodiment 43, wherein Xaa2 is selected from S, L, I, A, R, and K. 45. The engineered AAV VP capsid polypeptide of any one of embodiments 2-4, wherein Xaa3 has High mutability. 46. The engineered AAV VP capsid polypeptide of embodiment 45, wherein Xaa3 is selected from N, I, A, M, E, and D. 47. The engineered AAV VP capsid polypeptide of any one of embodiments 2-4, wherein Xaa3 has Low solubility. 48.

The engineered AAV VP capsid polypeptide of embodiment 47, wherein Xaa3 is selected from N, K, R, and E. 49. The engineered AAV VP capsid polypeptide of any one of embodiments 2-4, wherein Xaa4 has Low hydropathy. 50. The engineered AAV VP capsid polypeptide of embodiment 49, wherein Xaa4 is selected from K and R. 51. The engineered AAV VP capsid polypeptide of any one of embodiments 2-4, wherein Xaa4 has High amino acid volume. 52. The engineered AAV VP capsid polypeptide of embodiment 51, wherein Xaa4 is selected from K, R, I, and L. 53. The engineered AAV VP capsid polypeptide of any one of embodiments 2-4, wherein Xaa5 has Medium amino acid solubility. 54. The engineered AAV VP capsid polypeptide of embodiment 53, wherein Xaa5 is selected from H and T. 55. The engineered AAV VP capsid polypeptide of any one of embodiments 2-4, wherein Xaa8 has Low surface accessibility. 56. The engineered AAV VP capsid polypeptide of embodiment 55, wherein Xaa8 is selected from V and C. 57. The engineered AAV VP capsid polypeptide of any one of embodiments 2-4, wherein Xaa8 has Low average flexibility index. 58. The engineered AAV VP capsid polypeptide of embodiment 57, wherein Xaa8 is selected from W, V, M, A, F, L, H, and C. 59. The engineered AAV VP capsid polypeptide of any one of embodiments 1-58, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 44438-SEQ ID NO: 45437. 60. The engineered AAV VP capsid polypeptide of embodiment 59, wherein the region from the residue corresponding to residue 581 to the residue corresponding to residue 589 inclusive has a sequence of any one of SEQ ID NO: 44438-SEQ ID NO: 45437. 61. The engineered AAV VP capsid polypeptide of any one of embodiments 1-60, wherein tropism for liver tissue is measured as a relative accumulation of the rAAV virion in a liver tissue as compared to a non-liver tissue, wherein the non-liver tissue consists collectively of CNS, skeletal muscle, heart, lung, spleen, lymph node, bone marrow, mammary gland, skin, adrenal gland, thyroid, colon, sciatic nerve, and spinal cord.
62. The engineered AAV VP capsid polypeptide of embodiment 61, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof. 63. The engineered AAV VP capsid polypeptide of any one of embodiments 61-62, wherein the higher tissue tropism is a 1.0005-fold to about a 1000-fold increased accumulation in the liver tissue as compared to a non-liver tissue. 64. The engineered AAV VP capsid polypeptide of embodiment 63, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the liver tissue as compared to a non-liver tissue.

### Series E Embodiments—Adrenal Gland Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for an adrenal gland tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid. 3. An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for an adrenal gland tissue as compared to an rAAV virion having a wildtype AAV5 VP capsid polypeptide of SEQ ID NO: 1. 5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 1118-SEQ ID NO: 4117. 6. The engineered AAV VP capsid polypeptide of embodiment 5, wherein the region from the residue corresponding to residue 581 to the residue corresponding to residue 589, inclusive, has a sequence of any one of SEQ ID NO: 1118-SEQ ID NO: 4117. 7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 1118-SEQ ID NO: 4117. 8. The engineered AAV VP capsid polypeptide of embodiment 7, wherein the Xaa1 to Xaa9 region of SEQ ID NO: 2 has a sequence of any one of SEQ ID NO: 1118-SEQ ID NO: 4117. 9. The engineered AAV VP capsid polypeptide of any one of embodiments 2-4, wherein:
   Xaa1 is selected from the group consisting of A, C, K, Q, R, S, and T; or
   Xaa2 is selected from the group consisting of A, C, I, S, T, and V; or
   Xaa3 is selected from the group consisting of A, F, G, K, M, Q, R, T, and V; or
   Xaa4 is selected from the group consisting of A, K, M, Q, R, and V; or
   Xaa5 is selected from the group consisting of F, I, L, M, R, T, V, and Y; or
   Xaa6 is selected from the group consisting of G, H, M, N, R, and S; or
   Xaa7 is selected from the group consisting of A, H, K, Q, R, S and V; or
   Xaa8 is selected from the group consisting of A, G, H, M, Q, and S; or
   Xaa9 is selected from the group consisting of A, E, N, P, R, S, and Y; or any combination thereof. 10. The engineered AAV VP capsid polypeptide of embodiment 9, wherein Xaa1 is selected from C, K, and R. 11. The engineered AAV VP capsid polypeptide of embodiment 9, wherein Xaa1 is C. 12. The engineered AAV VP capsid polypeptide of embodiment 9, wherein Xaa2 is selected from A, V, and T. 13. The engineered AAV VP capsid polypeptide of embodiment 9, wherein Xaa2 is V. 14. The engineered AAV VP capsid polypeptide of embodiment 9, wherein Xaa3 is selected from A, G, and M. 15. The engineered AAV VP capsid polypeptide of embodiment 9, wherein Xaa3 is M. 16. The engineered AAV VP capsid polypeptide of embodiment 9, wherein Xaa4 is selected from A, R, and K. 17. The engineered AAV VP capsid polypeptide of embodiment 9, wherein Xaa4 is K. 18. The engineered AAV VP capsid polypeptide of embodiment 9, wherein Xaa5 is selected from R, V, and Y. 19. The engineered AAV VP capsid polypeptide of embodiment 9, wherein Xaa5 is V. 20. The engineered AAV VP capsid polypeptide of embodiment 9, wherein Xaa6 is selected from H and N. 21. The engineered AAV VP capsid polypeptide of embodiment 9, wherein Xaa6 is N. 22. The engineered AAV VP capsid polypeptide of embodiment 9, wherein Xaa7 is selected from H, Q, and V. 23. The engineered AAV VP capsid polypeptide of embodiment 9, wherein Xaa7 is H. 24. The engineered AAV VP capsid polypeptide of embodiment 9, wherein Xaa8 is selected from A, G, M, and S. 25. The engineered AAV VP capsid polypeptide of embodiment 9, wherein Xaa8 is S. 26. The engineered AAV VP capsid polypeptide of embodiment 9, wherein Xaa9 is selected from P and E. 27. The engineered AAV VP capsid polypeptide of embodiment 9, wherein Xaa9 is P. 28. The engineered AAV VP capsid polypeptide of any one of embodiments 1-27, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 1118-SEQ ID NO: 2117. 29.

The engineered AAV VP capsid polypeptide of embodiment 28, wherein the region from residue 581 to residue 589 of SEQ ID NO: 1 has a sequence of any one of SEQ ID NO: 1118-SEQ ID NO: 2117. 30. The engineered AAV VP capsid polypeptide of any one of embodiments 2-4, wherein Xaa1 has low mol mass. 31. The engineered AAV VP capsid polypeptide of embodiment 30, wherein Xaa1 is selected from V, P, S, and C. 32. The engineered AAV VP capsid polypeptide of any one of embodiments 2-4, wherein Xaa1 has low hydropathy. 33. The engineered AAV VP capsid polypeptide of embodiment X, wherein Xaa1 is selected from T, S, W, and Y. 34. The engineered AAV VP capsid polypeptide of any one of embodiments 2-4, wherein Xaa2 has low hydropathy. 35. The engineered AAV VP capsid polypeptide of embodiment 34, wherein Xaa2 is R. 36. The engineered AAV VP capsid polypeptide of any one of embodiments 2-4, wherein Xaa2 has low mutability. 37. The engineered AAV VP capsid polypeptide of embodiment 36, wherein Xaa2 is C. 38. The engineered AAV VP capsid polypeptide of any one of embodiments 2-4, wherein Xaa2 has low solubility. 39. The engineered AAV VP capsid polypeptide of embodiment 38, wherein Xaa2 is K. 40. The engineered AAV VP capsid polypeptide of any one of embodiments 2-4, wherein Xaa3 has low average flexibility. 41. The engineered AAV VP capsid polypeptide of embodiment 40, wherein Xaa3 is selected from W, M, and F. 42. The engineered AAV VP capsid polypeptide of any one of embodiments 2-4, wherein Xaa3 has high solubility. 43. The engineered AAV VP capsid polypeptide of embodiment 42, wherein Xaa3 is M. 44. The engineered AAV VP capsid polypeptide of any one of embodiments 2-4, wherein Xaa4 has high surface accessibility. 45. The engineered AAV VP capsid polypeptide of embodiment 44, wherein Xaa4 is selected from K and R. 46. The engineered AAV VP capsid polypeptide of any one of embodiments 2-4, wherein Xaa4 has high average flexibility. 47. The engineered AAV VP capsid polypeptide of embodiment 46, wherein Xaa4 is selected from K, I, and N.
48. The engineered AAV VP capsid polypeptide of any one of embodiments 2-4, wherein Xaa5 has medium mutability. 49. The engineered AAV VP capsid polypeptide of embodiment 48, wherein Xaa5 is selected from R and H. 50. The engineered AAV VP capsid polypeptide of any one of embodiments 2-4, wherein Xaa5 has high goldman engelman steitz. 51. The engineered AAV VP capsid polypeptide of embodiment 50, wherein Xaa5 is selected from V and L. 52. The engineered AAV VP capsid polypeptide of any one of embodiments 2-4, wherein Xaa5 has low hydropathy. 53. The engineered AAV VP capsid polypeptide of embodiment 52, wherein Xaa5 is R. 54. The engineered AAV VP capsid polypeptide of any one of embodiments 2-4, wherein Xaa5 has high volume.
55. The engineered AAV VP capsid polypeptide of embodiment 54, wherein Xaa5 is selected from Y, R, and F. 56. The engineered AAV VP capsid polypeptide of any one of embodiments 2-4, wherein Xaa6 has high solubility. 57. The engineered AAV VP capsid polypeptide of embodiment 56, wherein Xaa6 is selected from Y, V, M, A, and C. 58. The engineered AAV VP capsid polypeptide of any one of embodiments 2-4, wherein Xaa7 has medium mutability. 59. The engineered AAV VP capsid polypeptide of embodiment 58, wherein Xaa7 is selected from V, H, and R. 60. The engineered AAV VP capsid polypeptide of any one of embodiments 2-4, wherein Xaa7 has low solubility. 61. The engineered AAV VP capsid polypeptide of embodiment 59, wherein Xaa7 is R. 62. The engineered AAV VP capsid polypeptide of any one of embodiments 2-4, wherein Xaa8 has high average flexibility. 64. The engineered AAV VP capsid polypeptide of embodiment 62, wherein Xaa8 is selected from K, I, and N. 65. The engineered AAV VP capsid polypeptide of any one of embodiments 2-4, wherein Xaa8 has high mol mass. 66. The engineered AAV VP capsid polypeptide of embodiment 65, wherein Xaa8 is selected from R and Y. 67. The engineered AAV VP capsid polypeptide of any one of embodiments 2-4, wherein Xaa9 has high mutability. 68. The engineered AAV VP capsid polypeptide of embodiment X, wherein Xaa9 is N. 69. The engineered AAV VP capsid polypeptide of any one of embodiments 1-68, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 2118-SEQ ID NO: 3117. 70.

The engineered AAV VP capsid polypeptide of embodiment 69, wherein the region from the residue corresponding to residue 581 to the residue corresponding to residue 589 inclusive has a sequence of any one of SEQ ID NO: 2118-SEQ ID NO: 3117. 71. The engineered AAV VP capsid polypeptide of any one of embodiments 1-70, wherein tropism for adrenal gland tissue is measured as a relative accumulation of the rAAV virion in an adrenal gland tissue as compared to a non-adrenal gland tissue, wherein the non-adrenal gland tissue consists collectively of CNS, liver, skeletal muscle, heart, lung, spleen, lymph node, bone marrow, mammary gland, skin, thyroid, colon, sciatic nerve, and spinal cord. 72. The engineered AAV VP capsid polypeptide of embodiment 71, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof. 73. The engineered AAV VP capsid polypeptide of any one of embodiments 71-72, wherein the higher tissue tropism is a 1.0005-fold to about a 1000-fold increased accumulation in the adrenal gland tissue as compared to a non-adrenal gland tissue. 74. The engineered AAV VP capsid polypeptide of embodiment 73, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the adrenal gland tissue as compared to a non-adrenal gland tissue.

### Series F Embodiments—Bone Marrow Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a bone marrow tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid. 3. An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a bone marrow tissue as compared to an rAAV virion having a wildtype AAV5 VP capsid polypeptide of SEQ ID NO: 1. 5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 4118-SEQ ID NO: 7117. 6. The engineered AAV VP capsid polypeptide of embodiment 5, wherein the region from the residue corresponding to residue 581 to the residue corresponding to residue 589, inclusive, has a sequence of any one of SEQ ID NO: 4118-SEQ ID NO: 7117. 7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, at least 98%, or at least 99% identical to any one of SEQ ID NO: 4118-SEQ ID NO: 7117. 8. The engineered AAV VP capsid polypeptide of embodiment 7, wherein the Xaa1 to Xaa9 region of SEQ ID NO: 2 has a sequence of any one of SEQ ID NO: 4118-SEQ ID NO: 7117. 9. The engineered AAV VP capsid polypeptide of any one of embodiments 2-4, wherein:
   Xaa1 is selected from the group consisting of A, E, G, Q, S, and T; or
   Xaa2 is selected from the group consisting of A, I, Q, S, T, V, and Y; or
   Xaa3 is selected from the group consisting of A, G, I, M, Q, S, and T; or
   Xaa4 is selected from the group consisting of A, E, P, Q, T, and V; or
   Xaa5 is selected from the group consisting of F, I, L, M, Q, V, and Y; or
   Xaa6 is selected from the group consisting of F, I, N, Q, S, and V; or
   Xaa7 is selected from the group consisting of A, C, M, S, and V; or
   Xaa8 is selected from the group consisting of A, C, D, G, M, S, and Y; or
   Xaa9 is selected from the group consisting of D, E, G, L, P, S, and Y; or
any combination thereof. 10. The engineered AAV VP capsid polypeptide of embodiment 9, wherein Xaa1 is selected from A, E, and T. 11. The engineered AAV VP capsid polypeptide of embodiment 9, wherein Xaa1 is E. 12. The engineered AAV VP capsid polypeptide of embodiment 9, wherein Xaa2 is selected from A, S, and T. 13. The engineered AAV VP capsid polypeptide of embodiment 9, wherein Xaa2 is A. 14. The engineered AAV VP capsid polypeptide of embodiment 9, wherein Xaa3 is selected from A, Q, and T. 15. The engineered AAV VP capsid polypeptide of embodiment 9, wherein Xaa3 is Q. 16. The engineered AAV VP capsid polypeptide of embodiment 9, wherein Xaa4 is selected from A, P, and Q. 17. The engineered AAV VP capsid polypeptide of embodiment 9, wherein Xaa4 is Q. 18. The engineered AAV VP capsid polypeptide of embodiment 9, wherein Xaa5 is selected from F, V, and Y. 19. The engineered AAV VP capsid polypeptide of embodiment 9, wherein Xaa5 is V. 20. The engineered AAV VP capsid polypeptide of embodiment 9, wherein Xaa6 is selected from I, N, Q, and S. 21. The engineered AAV VP capsid polypeptide of embodiment 9, wherein Xaa6 is S. 22. The engineered AAV VP capsid polypeptide of embodiment 9, wherein Xaa7 is A, C, and V. 23. The engineered AAV VP capsid polypeptide of embodiment 9, wherein Xaa7 is C. 24. The engineered AAV VP capsid polypeptide of embodiment 9, wherein Xaa8 is selected from A, M, S, and Y. 25. The engineered AAV VP capsid polypeptide of embodiment 9, wherein Xaa8 is M. 26. The engineered AAV VP capsid polypeptide of embodiment 9, wherein Xaa9 is selected from D, E, and P. 27. The engineered AAV VP capsid polypeptide of embodiment 9, wherein Xaa9 is P. 28. The engineered AAV VP capsid polypeptide of any one of embodiments 1-27, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 4118-SEQ ID NO: 5117. 29. The engineered AAV VP capsid polypeptide of embodiment 28, wherein the region from residue 581 to residue 589 of SEQ ID NO: 1 has a sequence of any one of SEQ ID NO: 4118-SEQ ID NO: 5117. 30. The engineered AAV VP capsid polypeptide of any one of embodiments 2-4, wherein Xaa1 has high hydropathy. 31. The engineered AAV VP capsid polypeptide of embodiment 30, wherein Xaa1 is selected from V, I, and L. 32. The engineered AAV VP capsid polypeptide of any one of embodiments 2-4, wherein Xaa1 has low mutability. 33. The engineered AAV VP capsid polypeptide of embodiment 32, wherein Xaa1 is selected from Y, L, F, and C. 34. The engineered AAV VP capsid polypeptide of any one of embodiments 2-4, wherein Xaa2 has low hydropathy. 35. The engineered AAV VP capsid polypeptide of embodiment 34, wherein Xaa2 is selected from Y and W. 36. The engineered AAV VP capsid polypeptide of any one of embodiments 2-4, wherein Xaa2 has high mol mass. 37. The engineered AAV VP capsid polypeptide of embodiment 36, wherein Xaa2 is W. 38. The engineered AAV VP capsid polypeptide of any one of embodiments 2-4, wherein Xaa2 has low surface accessibility. 39. The engineered AAV VP capsid polypeptide of embodiment 38, wherein Xaa2 is selected from W and A. 40. The engineered AAV VP capsid polypeptide of any one of embodiments 2-4, wherein Xaa2 has low hydrophilicity. 41. The engineered AAV VP capsid polypeptide of embodiment 40, wherein Xaa2 is W. 42. The engineered AAV VP capsid polypeptide of any one of embodiments 2-4, wherein Xaa2 has low mutability. 43. The engineered AAV VP capsid polypeptide of embodiment 42, wherein Xaa2 is C. 44. The engineered AAV VP capsid polypeptide of any one of embodiments 2-4, wherein Xaa2 has low average flexibility. 45. The engineered AAV VP capsid polypeptide of embodiment 44, wherein Xaa2 is selected from W, M, and F. 46. The engineered AAV VP capsid polypeptide of any one of embodiments 2-4, wherein Xaa5 has low average flexibility. 47. The engineered AAV VP capsid polypeptide of embodiment 46, wherein Xaa5 is selected from W, M, and F. 48. The engineered AAV VP capsid polypeptide of any one of embodiments 2-4, wherein Xaa6 has low average flexibility.
49. The engineered AAV VP capsid polypeptide of embodiment 48, wherein Xaa6 is selected from W, M, and F. 50. The engineered AAV VP capsid polypeptide of any one of embodiments 2-4, wherein Xaa6 has low mutability. 51. The engineered AAV VP capsid polypeptide of embodiment 50, wherein Xaa6 is selected from Y, F, L, and C. 52. The engineered AAV VP capsid polypeptide of any one of embodiments 2-4, wherein Xaa6 has high solubility. 53. The engineered AAV VP capsid polypeptide of embodiment 52, wherein Xaa6 is selected from W, F, I, and L. 54. The engineered AAV VP capsid polypeptide of any one of embodiments 2-4, wherein Xaa7 has low surface accessibility. 55. The engineered AAV VP capsid polypeptide of embodiment 54, wherein Xaa7 is C. 56. The engineered AAV VP capsid polypeptide of any one of embodiments 2-4, wherein Xaa7 has high surface accessibility. 57. The engineered AAV VP capsid polypeptide of embodiment 56, wherein Xaa7 is selected from D and N.
58. The engineered AAV VP capsid polypeptide of any one of embodiments 2-4, wherein Xaa7 has low mutability.
59. The engineered AAV VP capsid polypeptide of embodiment 58, wherein Xaa7 is C.
60. The engineered AAV VP capsid polypeptide of any one of embodiments 2-4, wherein Xaa7 has high solubility.
61. The engineered AAV VP capsid polypeptide of embodiment 60, wherein Xaa7 is C.
62. The engineered AAV VP capsid polypeptide of any one of embodiments 2-4, wherein Xaa7 has low solubility.
63. The engineered AAV VP capsid polypeptide of embodiment X, wherein Xaa7 is D.
64. The engineered AAV VP capsid polypeptide of any one of embodiments 2-4, wherein Xaa8 has low charge.
65. The engineered AAV VP capsid polypeptide of embodiment 64, wherein Xaa8 is selected from D and E.
66. The engineered AAV VP capsid polypeptide of any one of embodiments 2-4, wherein Xaa8 has high mutability.
67. The engineered AAV VP capsid polypeptide of embodiment 66, wherein Xaa8 is selected from D, E, A, and T.
68. The engineered AAV VP capsid polypeptide of any one of embodiments 2-4, wherein Xaa9 has high mol mass.
69. The engineered AAV VP capsid polypeptide of embodiment 68, wherein Xaa9 is selected from H and F.
70. The engineered AAV VP capsid polypeptide of any one of embodiments 2-4, wherein Xaa9 has low mutability.
71. The engineered AAV VP capsid polypeptide of embodiment X, wherein Xaa9 is selected from Y, F, and L.
72. The engineered AAV VP capsid polypeptide of any one of embodiments 1-71, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 5118-SEQ ID NO: 6117.
73. The engineered AAV VP capsid polypeptide of embodiment 72, wherein the region from the residue corresponding to residue 581 to the residue corresponding to residue 589 inclusive has a sequence of any one of SEQ ID NO: 5118-SEQ ID NO: 6117.
74. The engineered AAV VP capsid polypeptide of any one of embodiments 1-73, wherein tropism for bone marrow tissue is measured as a relative accumulation of the rAAV virion in a bone marrow tissue as compared to a non-bone marrow tissue, wherein the non-bone marrow tissue consists collectively of CNS, liver, skeletal muscle, heart, lung, spleen, lymph node, mammary gland, skin, adrenal gland, thyroid, colon, sciatic nerve, and spinal cord.
75. The engineered AAV VP capsid polypeptide of embodiment 74, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.
76. The engineered AAV VP capsid polypeptide of any one of embodiments 74-75, wherein the higher tissue tropism is a 1.0005-fold to about a 1000-fold increased accumulation in the bone marrow tissue as compared to a non-bone marrow tissue.
77. The engineered AAV VP capsid polypeptide of embodiment 76, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the bone marrow tissue as compared to a non-bone marrow tissue.

### Series G Embodiments—Colon Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a colon tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid. 3. An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a colon tissue as compared to an rAAV virion having a wildtype AAV5 VP capsid polypeptide of SEQ ID NO: 1.
5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 10118-SEQ ID NO: 13117.
6. The engineered AAV VP capsid polypeptide of embodiment 5, wherein the region from the residue corresponding to residue 581 to the residue corresponding to residue 589, inclusive, has a sequence of any one of SEQ ID NO: 10118-SEQ ID NO: 13117.
7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 10118-SEQ ID NO: 13117.
8. The engineered AAV VP capsid polypeptide of embodiment 7, wherein the Xaa1 to Xaa9 region of SEQ ID NO: 2 has a sequence of any one of SEQ ID NO: 10118-SEQ ID NO: 13117.
9. The engineered AAV VP capsid polypeptide of any one of embodiments 2-4, wherein:
   Xaa1 is selected from the group consisting of C, F, H, N, P, W, and Y; or
   Xaa2 is selected from the group consisting of D, E, F, L, and P; or
   Xaa3 is selected from the group consisting of C, F, H, I, L, P, and Y; or
   Xaa4 is selected from the group consisting of C, D, E, N, and P; or
   Xaa5 is selected from the group consisting of D, E, G, P, and W; or
   Xaa6 is selected from the group consisting of C, K, R, and V; or
   Xaa7 is selected from the group consisting of D, M, P, and V; or
   Xaa8 is selected from the group consisting of D, I, K, L, P, R, and V; or
   Xaa9 is selected from the group consisting of C, H, I, K, L, M, and W; or
   any combination thereof.
10. The engineered AAV VP capsid polypeptide of embodiment 9, wherein Xaa1 is selected from F, P, and W.
11. The engineered AAV VP capsid polypeptide of embodiment 9, wherein Xaa1 is P.
12. The engineered AAV VP capsid polypeptide of embodiment 9, wherein Xaa2 is selected from D, E, L, and P.
13. The engineered AAV VP capsid polypeptide of embodiment 9, wherein Xaa2 is P.
14. The engineered AAV VP capsid polypeptide of embodiment 9, wherein Xaa3 is selected from C, H, and P.
15. The engineered AAV VP capsid polypeptide of embodiment 9, wherein Xaa3 is P.
16. The engineered AAV VP capsid polypeptide of embodiment 9, wherein Xaa4 is selected from C, D, and E.
17. The engineered AAV VP capsid polypeptide of embodiment 9, wherein Xaa4 is C.
18. The engineered AAV VP capsid polypeptide of embodiment 9, wherein Xaa5 is selected from G, P, and W.
19. The engineered AAV VP capsid polypeptide of embodiment 9, wherein Xaa5 is P.
20. The engineered AAV VP capsid polypeptide of embodiment 9, wherein Xaa6 is selected from K and R.
21. The engineered AAV VP capsid polypeptide of embodiment 9, wherein Xaa6 is R.
22. The engineered AAV VP capsid polypeptide of embodiment 9, wherein Xaa7 is P.
23. The engineered AAV VP capsid polypeptide of embodiment 9, wherein Xaa8 is selected from K, P, and R.
24. The engineered AAV VP capsid polypeptide of embodiment 9, wherein Xaa8 is P.
25. The engineered AAV VP capsid polypeptide of embodiment 9, wherein Xaa9 is selected from I, L, and M.
26. The engineered AAV VP capsid polypeptide of embodiment 9, wherein Xaa9 is I.
27. The engineered AAV VP capsid polypeptide of any one of embodiments 1-26, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 10118-SEQ ID NO: 11117.
28. The engineered AAV VP capsid polypeptide of embodiment 27, wherein the region from residue 581 to residue 589 of SEQ ID NO: 1 has a sequence of any one of SEQ ID NO: 10118-SEQ ID NO: 11117.
29. The engineered AAV VP capsid polypeptide of any one of embodiments 2-4, wherein Xaa1 has High mol mass.
30. The engineered AAV VP capsid polypeptide of embodiment 29, wherein Xaa1 is selected from Y, W.
31. The engineered AAV VP capsid polypeptide of any one of embodiments 2-4, wherein Xaa1 has High solubility.
32. The engineered AAV VP capsid polypeptide of embodiment 31, wherein Xaa1 is selected from W, F, I, and L.
33. The engineered AAV VP capsid polypeptide of any one of embodiments 2-4, wherein Xaa2 has Low solubility.
34. The engineered AAV VP capsid polypeptide of embodiment 33, wherein Xaa2 is D.
35. The engineered AAV VP capsid polypeptide of any one of embodiments 2-4, wherein Xaa2 has Low mutability.
36. The engineered AAV VP capsid polypeptide of embodiment 35, wherein Xaa2 is selected from P and K.
37. The engineered AAV VP capsid polypeptide of any one of embodiments 2-4, wherein Xaa2 has Medium mol mass.
38. The engineered AAV VP capsid polypeptide of embodiment 37, wherein Xaa2 is selected from D, E, N, K, M, Q, I, and L.
39. The engineered AAV VP capsid polypeptide of any one of embodiments 2-4, wherein Xaa2 has Low hydropathy.
40. The engineered AAV VP capsid polypeptide of embodiment 39, wherein Xaa2 is selected from D, E, R, K, H, N, and Q.
41. The engineered AAV VP capsid polypeptide of any one of embodiments 2-4, wherein Xaa3 has Low mutability.
42. The engineered AAV VP capsid polypeptide of embodiment 41, wherein Xaa3 is selected from K, V, P, and C.
43. The engineered AAV VP capsid polypeptide of any one of embodiments 2-4, wherein Xaa3 has High solubility.
44. The engineered AAV VP capsid polypeptide of embodiment 43, wherein Xaa3 is selected from W, F, I, and L.
45. The engineered AAV VP capsid polypeptide of any one of embodiments 2-4, wherein Xaa5 has High average flexibility.
46. The engineered AAV VP capsid polypeptide of embodiment 45, wherein Xaa5 is selected from S, P, G, R, E, and D.
47. The engineered AAV VP capsid polypeptide of any one of embodiments 2-4, wherein Xaa5 has High surface accessibility.
48. The engineered AAV VP capsid polypeptide of embodiment 47, wherein Xaa5 is selected from D and N.
49. The engineered AAV VP capsid polypeptide of any one of embodiments 2-4, wherein Xaa6 has Low hydropathy.
50. The engineered AAV VP capsid polypeptide of embodiment 49, wherein Xaa6 is selected from R.
51. The engineered AAV VP capsid polypeptide of any one of embodiments 2-4, wherein Xaa6 has Low mutability.
52. The engineered AAV VP capsid polypeptide of embodiment 51, wherein Xaa6 is selected from Y, R, F, and L.
53. The engineered AAV VP capsid polypeptide of any one of embodiments 2-4, wherein Xaa6 has Low solubility.
54. The engineered AAV VP capsid polypeptide of embodiment 53, wherein Xaa6 is selected from R and Q.
55. The engineered AAV VP capsid polypeptide of any one of embodiments 2-4, wherein Xaa6 has High surface accessibility.
56. The engineered AAV VP capsid polypeptide of embodiment 55, wherein Xaa6 is selected from E, R, K.
57. The engineered AAV VP capsid polypeptide of any one of embodiments 2-4, wherein Xaa6 has High average flexibility.
58. The engineered AAV VP capsid polypeptide of embodiment 57, wherein Xaa6 is selected from G and R.
59. The engineered AAV VP capsid polypeptide of any one of embodiments 2-4, wherein Xaa8 has Low solubility.
60. The engineered AAV VP capsid polypeptide of embodiment 59, wherein Xaa8 is D.
61. The engineered AAV VP capsid polypeptide of any one of embodiments 1-60, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 11118-SEQ ID NO: 12117.
62. The engineered AAV VP capsid polypeptide of embodiment 61, wherein the region from the residue corresponding to residue 581 to the residue corresponding to residue 589 inclusive has a sequence of any one of SEQ ID NO: 11118-SEQ ID NO: 12117.
63. The engineered AAV VP capsid polypeptide of any one of embodiments 1-62, wherein tropism for colon tissue is measured as a relative accumulation of the rAAV virion in a colon tissue as compared to a non-colon tissue, wherein the non-colon tissue consists collectively of CNS, liver, skeletal muscle, heart, lung, spleen, lymph node, bone marrow, mammary gland, skin, adrenal gland, thyroid, sciatic nerve, and spinal cord.
64. The engineered AAV VP capsid polypeptide of embodiment 63, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.
65. The engineered AAV VP capsid polypeptide of any one of embodiments 63-64, wherein the higher tissue tropism is a 1.0005-fold to about a 1000-fold increased accumulation in the colon tissue as compared to a non-colon tissue.
66. The engineered AAV VP capsid polypeptide of embodiment 65, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the colon tissue as compared to a non-colon tissue.

### Series H Embodiments—Heart Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a heart tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid.
3. An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a heart tissue as compared to an rAAV virion having a wildtype AAV5 VP capsid polypeptide of SEQ ID NO: 1.
5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 13118-SEQ ID NO: 16117.
6. The engineered AAV VP capsid polypeptide of embodiment 5, wherein the region from the residue corresponding to residue 581 to the residue corresponding to residue 589, inclusive, has a sequence of any one of SEQ ID NO: 13118-SEQ ID NO: 16117.
7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 13118-SEQ ID NO: 16117.
8. The engineered AAV VP capsid polypeptide of embodiment 7, wherein the Xaa1 to Xaa9 region of SEQ ID NO: 2 has a sequence of any one of SEQ ID NO: 13118-SEQ ID NO: 16117.
9. The engineered AAV VP capsid polypeptide of any one of embodiments 2-4, wherein:
Xaa1 is selected from the group consisting of I, K, L, M, T, and V; or
Xaa2 is selected from the group consisting of A, C, G, I, K, and S; or
Xaa3 is selected from the group consisting of A, D, E, G, K, M, and V; or
Xaa4 is selected from the group consisting of F, H, R, T, W, and Y; or
Xaa5 is selected from the group consisting of F, L, M, and R; or
Xaa6 is selected from the group consisting of A, H, N, W, and Y; or
Xaa7 is selected from the group consisting of A, C, E, F, K, and T; or
Xaa8 is selected from the group consisting of A, C, M, S, and T; or
Xaa9 is selected from the group consisting of A, D, G, and P; or
any combination thereof.
10. The engineered AAV VP capsid polypeptide of embodiment 9, wherein Xaa1 is selected from K and L.
11. The engineered AAV VP capsid polypeptide of embodiment 9, wherein Xaa1 is K.
12. The engineered AAV VP capsid polypeptide of embodiment 9, wherein Xaa2 is selected from A, C, and S.
13. The engineered AAV VP capsid polypeptide of embodiment 9, wherein Xaa2 is A.
14. The engineered AAV VP capsid polypeptide of embodiment 9, wherein Xaa3 is selected from E and V.
15. The engineered AAV VP capsid polypeptide of embodiment 9, wherein Xaa3 is E.
16. The engineered AAV VP capsid polypeptide of embodiment 9, wherein Xaa4 is selected from F, R, and T.
17. The engineered AAV VP capsid polypeptide of embodiment 9, wherein Xaa4 is R.
18. The engineered AAV VP capsid polypeptide of embodiment 9, wherein Xaa5 is L.
19. The engineered AAV VP capsid polypeptide of embodiment 9, wherein Xaa6 is selected from H, N, and Y.
20. The engineered AAV VP capsid polypeptide of embodiment 9, wherein Xaa6 is H.
21. The engineered AAV VP capsid polypeptide of embodiment 9, wherein Xaa7 is selected from C, F, and T.
22. The engineered AAV VP capsid polypeptide of embodiment 9, wherein Xaa7 is F.
23. The engineered AAV VP capsid polypeptide of embodiment 9, wherein Xaa8 is selected from C, M, and S.
24. The engineered AAV VP capsid polypeptide of embodiment 9, wherein Xaa8 is C.
25. The engineered AAV VP capsid polypeptide of embodiment 9, wherein Xaa9 is selected from A and G.
26. The engineered AAV VP capsid polypeptide of embodiment 9, wherein Xaa9 is A.
27. The engineered AAV VP capsid polypeptide of any one of embodiments 1-26, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 13118-SEQ ID NO: 14117.
28. The engineered AAV VP capsid polypeptide of embodiment 27, wherein the region from residue 581 to residue 589 of SEQ ID NO: 1 has a sequence of any one of SEQ ID NO: 13118-SEQ ID NO: 14117.
29. The engineered AAV VP capsid polypeptide of any one of embodiments 2-4, wherein Xaa1 has low solubility.
30. The engineered AAV VP capsid polypeptide of embodiment 29, wherein Xaa1 is selected from N and E.
31. The engineered AAV VP capsid polypeptide of any one of embodiments 2-4, wherein Xaa1 has low hydropathy.
32. The engineered AAV VP capsid polypeptide of embodiment 31, wherein Xaa1 is selected from H, N, Q, P, Y, D, and E.
33. The engineered AAV VP capsid polypeptide of any one of embodiments 2-4, wherein Xaa1 has high mutability.
34. The engineered AAV VP capsid polypeptide of embodiment 33, wherein Xaa1 is selected from A and E.
35. The engineered AAV VP capsid polypeptide of any one of embodiments 2-4, wherein Xaa2 has high hydropathy.
36. The engineered AAV VP capsid polypeptide of embodiment 35, wherein Xaa2 is selected from V and I.
37. The engineered AAV VP capsid polypeptide of any one of embodiments 2-4, wherein Xaa2 has medium mutability.
38. The engineered AAV VP capsid polypeptide of embodiment 37, wherein Xaa2 is V.
39. The engineered AAV VP capsid polypeptide of any one of embodiments 2-4, wherein Xaa2 has medium volume.
40. The engineered AAV VP capsid polypeptide of embodiment 39, wherein Xaa2 is selected from V, E, and Q.
41. The engineered AAV VP capsid polypeptide of any one of embodiments 2-4, wherein Xaa2 has high solubility.
42. The engineered AAV VP capsid polypeptide of embodiment 41, wherein Xaa2 is selected from V and M.
43. The engineered AAV VP capsid polypeptide of any one of embodiments 2-4, wherein Xaa3 has low solubility.
44. The engineered AAV VP capsid polypeptide of embodiment 43, wherein Xaa3 is selected from R and Q.
45. The engineered AAV VP capsid polypeptide of any one of embodiments 2-4, wherein Xaa4 has low surface accessibility.
46. The engineered AAV VP capsid polypeptide of embodiment 45, wherein Xaa4 is C.
47. The engineered AAV VP capsid polypeptide of any one of embodiments 2-4, wherein Xaa4 has high solubility.
48. The engineered AAV VP capsid polypeptide of embodiment 47, wherein Xaa4 is C.
49. The engineered AAV VP capsid polypeptide of any one of embodiments 2-4, wherein Xaa4 has low charge.
50. The engineered AAV VP capsid polypeptide of embodiment 49, wherein Xaa4 is selected from D, E, Y, W, V, P, M, A, G, F, I, L, N, Q, S, T, and C.
51. The engineered AAV VP capsid polypeptide of any one of embodiments 2-4, wherein Xaa4 has high hydropathy.
52. The engineered AAV VP capsid polypeptide of embodiment 51, wherein Xaa4 is C.
53. The engineered AAV VP capsid polypeptide of any one of embodiments 2-4, wherein Xaa5 has high surface accessibility.
54. The engineered AAV VP capsid polypeptide of embodiment 53, wherein Xaa5 is selected from D, E, R, K, N, and Q.
55. The engineered AAV VP capsid polypeptide of any one of embodiments 2-4, wherein Xaa5 has low solubility.
56. The engineered AAV VP capsid polypeptide of embodiment 55, wherein Xaa5 is D.
57. The engineered AAV VP capsid polypeptide of any one of embodiments 2-4, wherein Xaa6 has low mutability.
58. The engineered AAV VP capsid polypeptide of embodiment 57, wherein Xaa6 is C.
59. The engineered AAV VP capsid polypeptide of any one of embodiments 2-4, wherein Xaa6 has low solubility.
60. The engineered AAV VP capsid polypeptide of embodiment 59, wherein Xaa6 is D.
61. The engineered AAV VP capsid polypeptide of any one of embodiments 2-4, wherein Xaa8 has high surface accessibility.
62. The engineered AAV VP capsid polypeptide of embodiment 61, wherein Xaa8 is selected from D and N.
63. The engineered AAV VP capsid polypeptide of any one of embodiments 2-4, wherein Xaa8 has high average flexibility.
64. The engineered AAV VP capsid polypeptide of embodiment 63, wherein Xaa8 is selected from D, R, P, G, and S.
65. The engineered AAV VP capsid polypeptide of any one of embodiments 2-4, wherein Xaa9 has medium mol mass.
66. The engineered AAV VP capsid polypeptide of embodiment 65, wherein Xaa9 is selected from N, D, L, and I.
67. The engineered AAV VP capsid polypeptide of any one of embodiments 1-66, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 14118-SEQ ID NO: 15117.
68. The engineered AAV VP capsid polypeptide of embodiment 67, wherein the region from the residue corresponding to residue 581 to the residue corresponding to residue 589 inclusive has a sequence of any one of SEQ ID NO: 14118-SEQ ID NO: 15117.
69. The engineered AAV VP capsid polypeptide of any one of embodiments 1-68, wherein tropism for heart tissue is measured as a relative accumulation of the rAAV virion in a heart tissue as compared to a non-heart tissue, wherein the non-heart tissue consists collectively of CNS, liver, skeletal muscle, lung, spleen, lymph node, bone marrow, mammary gland, skin, adrenal gland, thyroid, colon, sciatic nerve, and spinal cord.
70. The engineered AAV VP capsid polypeptide of embodiment 69, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.
71. The engineered AAV VP capsid polypeptide of any one of embodiments 69-70, wherein the higher tissue tropism is a 1.0005-fold to about a 1000-fold increased accumulation in the heart tissue as compared to a non-heart tissue.
72. The engineered AAV VP capsid polypeptide of embodiment 71, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the heart tissue as compared to a non-heart tissue.

### Series I Embodiments—Lung Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a lung tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid. 3. An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8.
4. The engineered AAV VP capsid polypeptide of embodiment 3, wherein the rAAV has higher tropism for a lung tissue as compared to an rAAV virion having a wildtype AAV5 VP capsid polypeptide of SEQ ID NO: 1.
5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 16118-SEQ ID NO: 19117.
6. The engineered AAV VP capsid polypeptide of embodiment 5, wherein the region from the residue corresponding to residue 581 to the residue corresponding to residue 589, inclusive, has a sequence of any one of SEQ ID NO: 16118-SEQ ID NO: 19117.
7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 16118-SEQ ID NO: 19117.
8. The engineered AAV VP capsid polypeptide of embodiment 7, wherein the Xaa1 to Xaa9 region of SEQ ID NO: 2 has a sequence of any one of SEQ ID NO: 16118-SEQ ID NO: 19117.
9. The engineered AAV VP capsid polypeptide of any one of embodiments 2-4, wherein: Xaa1 is selected from the group consisting of A, E, K, M, Q, R, S, and T; or
Xaa2 is selected from the group consisting of A, I, K, S, T, and V; or
Xaa3 is selected from the group consisting of A, E, K, M, Q, R, S, T, and V; or
Xaa4 is selected from the group consisting of M, P, R, S, and T; or
Xaa5 is selected from the group consisting of I, K, L, M, T, V, and Y; or
Xaa6 is selected from the group consisting of D, G, H, M, N, R, and S; or
Xaa7 is selected from the group consisting of A, K, M, Q, and R; or
Xaa8 is selected from the group consisting of A, F, G, S, W, and Y; or
Xaa9 is selected from the group consisting of A, E, G, P, R, and Y; or
any combination thereof.
10. The engineered AAV VP capsid polypeptide of embodiment 9, wherein Xaa1 is selected from A, E, and Q.
11. The engineered AAV VP capsid polypeptide of embodiment 9, wherein Xaa1 is E.
12. The engineered AAV VP capsid polypeptide of embodiment 9, wherein Xaa2 is selected from S, T, and V.
13. The engineered AAV VP capsid polypeptide of embodiment 9, wherein Xaa2 is T.
14. The engineered AAV VP capsid polypeptide of embodiment 9, wherein Xaa3 is selected from A, K, R, and S.
15. The engineered AAV VP capsid polypeptide of embodiment 9, wherein Xaa3 is R.
16. The engineered AAV VP capsid polypeptide of embodiment 9, wherein Xaa4 is selected from P, Q, and T.
17. The engineered AAV VP capsid polypeptide of embodiment 9, wherein Xaa4 is Q.
18. The engineered AAV VP capsid polypeptide of embodiment 9, wherein Xaa5 is selected from L, M, and Y.
19. The engineered AAV VP capsid polypeptide of embodiment 9, wherein Xaa5 is L.
20. The engineered AAV VP capsid polypeptide of embodiment 9, wherein Xaa6 is selected from H and N.
21. The engineered AAV VP capsid polypeptide of embodiment 9, wherein Xaa6 is N.
22. The engineered AAV VP capsid polypeptide of embodiment 9, wherein Xaa7 is selected from A, K and R.
23. The engineered AAV VP capsid polypeptide of embodiment 9, wherein Xaa7 is R.
24. The engineered AAV VP capsid polypeptide of embodiment 9, wherein Xaa8 is selected from A, F, and G.
25. The engineered AAV VP capsid polypeptide of embodiment 9, wherein Xaa8 is F.
26. The engineered AAV VP capsid polypeptide of embodiment 9, wherein Xaa9 is selected from G, P, and R.
27. The engineered AAV VP capsid polypeptide of embodiment 9, wherein Xaa9 is G.
28. The engineered AAV VP capsid polypeptide of any one of embodiments 1-27, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 16118-SEQ ID NO: 17117.
29. The engineered AAV VP capsid polypeptide of embodiment 28, wherein the region from residue 581 to residue 589 of SEQ ID NO: 1 has a sequence of any one of SEQ ID NO: 16118-SEQ ID NO: 17117.
30. The engineered AAV VP capsid polypeptide of any one of embodiments 2-4, wherein Xaa1 has high mutability.
31. The engineered AAV VP capsid polypeptide of embodiment 30, wherein Xaa1 is selected from D, E, M, A, I, Q, and T.
32. The engineered AAV VP capsid polypeptide of any one of embodiments 2-4, wherein Xaa2 has high mol mass.
33. The engineered AAV VP capsid polypeptide of embodiment 32, wherein Xaa2 is F.
34. The engineered AAV VP capsid polypeptide of any one of embodiments 2-4, wherein Xaa2 has low mutability.
35. The engineered AAV VP capsid polypeptide of embodiment 34, wherein Xaa2 is selected from Y, F, and L.
36. The engineered AAV VP capsid polypeptide of any one of embodiments 2-4, wherein Xaa3 has low mutability.
37. The engineered AAV VP capsid polypeptide of embodiment 36, wherein Xaa3 is selected from K, V, P, and H.
38. The engineered AAV VP capsid polypeptide of any one of embodiments 2-4, wherein Xaa3 has low hydropathy.
39. The engineered AAV VP capsid polypeptide of embodiment 38, wherein Xaa3 is selected from K and R.
40. The engineered AAV VP capsid polypeptide of any one of embodiments 2-4, wherein Xaa4 has low mutability.
41. The engineered AAV VP capsid polypeptide of embodiment 40, wherein Xaa4 is selected from K and P.
42. The engineered AAV VP capsid polypeptide of any one of embodiments 2-4, wherein Xaa4 has high average flexibility.
43. The engineered AAV VP capsid polypeptide of embodiment 42, wherein Xaa4 is selected from D, E, P, and S.
44. The engineered AAV VP capsid polypeptide of any one of embodiments 2-4, wherein Xaa5 has low average flexibility.
45. The engineered AAV VP capsid polypeptide of embodiment 44, wherein Xaa5 is selected from W, M, and F.
46. The engineered AAV VP capsid polypeptide of any one of embodiments 2-4, wherein Xaa5 has high solubility.
47. The engineered AAV VP capsid polypeptide of embodiment 46, wherein Xaa5 is selected from W, F, I, and L.
48. The engineered AAV VP capsid polypeptide of any one of embodiments 2-4, wherein Xaa6 has medium mutability.
49. The engineered AAV VP capsid polypeptide of embodiment 48, wherein Xaa6 is selected from R, and H.
50. The engineered AAV VP capsid polypeptide of any one of embodiments 2-4, wherein Xaa6 has high surface accessibility.
51. The engineered AAV VP capsid polypeptide of embodiment 50, wherein Xaa6 is selected from T.
52. The engineered AAV VP capsid polypeptide of any one of embodiments 2-4, wherein Xaa7 has low mutability.
53. The engineered AAV VP capsid polypeptide of embodiment 52, wherein Xaa7 is C.
54. The engineered AAV VP capsid polypeptide of any one of embodiments 2-4, wherein Xaa7 has high solubility.
55. The engineered AAV VP capsid polypeptide of embodiment 54, wherein Xaa7 is selected from W, V, M, F, I, and L.
56. The engineered AAV VP capsid polypeptide of any one of embodiments 2-4, wherein Xaa8 has high mutability.
57. The engineered AAV VP capsid polypeptide of embodiment 56, wherein Xaa8 is selected from D, E, M, A, I, Q, and T.
58. The engineered AAV VP capsid polypeptide of any one of embodiments 2-4, wherein Xaa8 has low hydropathy.
59. The engineered AAV VP capsid polypeptide of embodiment 58, wherein Xaa8 is selected from R and K.
60. The engineered AAV VP capsid polypeptide of any one of embodiments 2-4, wherein Xaa9 has high average flexibility.
61. The engineered AAV VP capsid polypeptide of embodiment 60, wherein Xaa9 is selected from R and G.
62. The engineered AAV VP capsid polypeptide of any one of embodiments 1-61, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 17118-SEQ ID NO: 18117.
63. The engineered AAV VP capsid polypeptide of embodiment 62, wherein the region from the residue corresponding to residue 581 to the residue corresponding to residue 589 inclusive has a sequence of any one of SEQ ID NO: 17118-SEQ ID NO: 18117.
64. The engineered AAV VP capsid polypeptide of any one of embodiments 1-63, wherein tropism for lung tissue is measured as a relative accumulation of the rAAV virion in a lung tissue as compared to a non-lung tissue, wherein the non-lung tissue consists collectively of CNS, liver, skeletal muscle, spleen, lymph node, bone marrow, mammary gland, skin, adrenal gland, thyroid, colon, sciatic nerve, and spinal cord.
65. The engineered AAV VP capsid polypeptide of embodiment 64, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.
66. The engineered AAV VP capsid polypeptide of any one of embodiments 64-65, wherein the higher tissue tropism is a 1.0005-fold to about a 1000-fold increased accumulation in the lung tissue as compared to a non-lung tissue.
67. The engineered AAV VP capsid polypeptide of embodiment 66, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the lung tissue as compared to a non-lung tissue.

### Series J Embodiments—Lymph Node Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a lymph node tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid. 3. An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a lymph node tissue as compared to an rAAV virion having a wildtype AAV5 VP capsid polypeptide of SEQ ID NO: 1. 5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 19118-SEQ ID NO: 22117. 6. The engineered AAV VP capsid polypeptide of embodiment 5, wherein the region from the residue corresponding to residue 581 to the residue corresponding to residue 589, inclusive, has a sequence of any one of SEQ ID NO: 19118-SEQ ID NO: 22117. 7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 19118-SEQ ID NO: 22117. 8. The engineered AAV VP capsid polypeptide of embodiment 7, wherein the Xaa1 to Xaa9 region of SEQ ID NO: 2 has a sequence of any one of SEQ ID NO: 19118-SEQ ID NO: 22117. 9. The engineered AAV VP capsid polypeptide of any one of embodiments 2-4, wherein:
   Xaa1 is selected from the group consisting of A, D, E, Q, S, and T; or
   Xaa2 is selected from the group consisting of A, H, I, S, T, and V; or
   Xaa3 is selected from the group consisting of A, E, H, I, T, and V; or
   Xaa4 is selected from the group consisting of A, D, E, and P; or
   Xaa5 is selected from the group consisting of I, L, M, V, and Y; or
   Xaa6 is selected from the group consisting of D, E, I, N, and Q; or
   Xaa7 is selected from the group consisting of A, E, G, Q, and V; or
   Xaa8 is selected from the group consisting of F, G, M, and W; or
   Xaa9 is selected from the group consisting of I, P, T, and Y; or
   any combination thereof.
10. The engineered AAV VP capsid polypeptide of embodiment 9, wherein Xaa1 is selected from D, E, and T.
11. The engineered AAV VP capsid polypeptide of embodiment 9, wherein Xaa1 is E.
12. The engineered AAV VP capsid polypeptide of embodiment 9, wherein Xaa2 is selected from I, T, and V.
13. The engineered AAV VP capsid polypeptide of embodiment 9, wherein Xaa2 is V.
14. The engineered AAV VP capsid polypeptide of embodiment 9, wherein Xaa3 is selected from A, I, T, and V.
15. The engineered AAV VP capsid polypeptide of embodiment 9, wherein Xaa3 is T.
16. The engineered AAV VP capsid polypeptide of embodiment 9, wherein Xaa4 is selected from D and E.
17. The engineered AAV VP capsid polypeptide of embodiment 9, wherein Xaa4 is E.
18. The engineered AAV VP capsid polypeptide of embodiment 9, wherein Xaa5 is selected from I, L, V, and Y.
19. The engineered AAV VP capsid polypeptide of embodiment 9, wherein Xaa5 is L.
20. The engineered AAV VP capsid polypeptide of embodiment 9, wherein Xaa6 is selected from D, E, and I.
21. The engineered AAV VP capsid polypeptide of embodiment 9, wherein Xaa6 is D.
22. The engineered AAV VP capsid polypeptide of embodiment 9, wherein Xaa7 is A, Q, or V.
23. The engineered AAV VP capsid polypeptide of embodiment 9, wherein Xaa7 is V.
24. The engineered AAV VP capsid polypeptide of embodiment 9, wherein Xaa8 is selected from F and W.
25. The engineered AAV VP capsid polypeptide of embodiment 9, wherein Xaa8 is W.
26. The engineered AAV VP capsid polypeptide of embodiment 9, wherein Xaa9 is I or P.
27. The engineered AAV VP capsid polypeptide of any one of embodiments 1-26, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 19118-SEQ ID NO: 20117.
28. The engineered AAV VP capsid polypeptide of embodiment 27, wherein the region from residue 581 to residue 589 of SEQ ID NO: 1 has a sequence of any one of SEQ ID NO: 19118-SEQ ID NO: 20117.
29. The engineered AAV VP capsid polypeptide of any one of embodiments 2-4, wherein Xaa1 has high average flexibility.
30. The engineered AAV VP capsid polypeptide of embodiment 29, wherein Xaa1 is selected from D, E, P, G, Q, S, and R.
31. The engineered AAV VP capsid polypeptide of any one of embodiments 2-4, wherein Xaa1 has high hbond donors.
32. The engineered AAV VP capsid polypeptide of embodiment 31, wherein Xaa1 is R.
33. The engineered AAV VP capsid polypeptide of any one of embodiments 2-4, wherein Xaa1 has high mol mass.
34. The engineered AAV VP capsid polypeptide of embodiment 33, wherein Xaa1 is selected from Y, W, R, and F.
35. The engineered AAV VP capsid polypeptide of any one of embodiments 2-4, wherein Xaa2 has low solubility.
36. The engineered AAV VP capsid polypeptide of embodiment 35, wherein Xaa2 is selected from N and E.
37. The engineered AAV VP capsid polypeptide of any one of embodiments 2-4, wherein Xaa3 has low average flexibility.
38. The engineered AAV VP capsid polypeptide of embodiment 37, wherein Xaa3 is selected from W, M, and F.
39. The engineered AAV VP capsid polypeptide of any one of embodiments 2-4, wherein Xaa3 has low mutability.
40. The engineered AAV VP capsid polypeptide of embodiment 39, wherein Xaa3 is selected from R, H, K, P, Y, F, L, and C.
41. The engineered AAV VP capsid polypeptide of any one of embodiments 2-4, wherein Xaa4 has low mutability.
42. The engineered AAV VP capsid polypeptide of embodiment 41, wherein Xaa4 is C.
43. The engineered AAV VP capsid polypeptide of any one of embodiments 2-4, wherein Xaa5 has high mutability.
44. The engineered AAV VP capsid polypeptide of embodiment 43, wherein Xaa5 is N.
45. The engineered AAV VP capsid polypeptide of any one of embodiments 2-4, wherein Xaa5 has medium mol mass.
46. The engineered AAV VP capsid polypeptide of embodiment 45, wherein Xaa5 is selected from D, I, L, and N.
47. The engineered AAV VP capsid polypeptide of any one of embodiments 2-4, wherein Xaa6 has high mol mass.
48. The engineered AAV VP capsid polypeptide of embodiment 47, wherein Xaa6 is selected from Y, W, R, and F.
49. The engineered AAV VP capsid polypeptide of any one of embodiments 2-4, wherein Xaa6 has high average flexibility.
50. The engineered AAV VP capsid polypeptide of embodiment 49, wherein Xaa6 is selected from G and R.
51. The engineered AAV VP capsid polypeptide of any one of embodiments 2-4, wherein Xaa7 has high average flexibility.
52. The engineered AAV VP capsid polypeptide of embodiment 51, wherein Xaa7 is selected from D, E, K, P, I, N, Q, and S.
53. The engineered AAV VP capsid polypeptide of any one of embodiments 2-4, wherein Xaa7 has low solubility.
54. The engineered AAV VP capsid polypeptide of embodiment 53, wherein Xaa7 is selected from N and E.
55. The engineered AAV VP capsid polypeptide of any one of embodiments 2-4, wherein Xaa8 has low solubility.
56. The engineered AAV VP capsid polypeptide of embodiment 55, wherein Xaa8 is selected from N, E, and D.
57. The engineered AAV VP capsid polypeptide of any one of embodiments 2-4, wherein Xaa8 has medium mutability.
58. The engineered AAV VP capsid polypeptide of embodiment 57, wherein Xaa8 is selected from R and H.
59. The engineered AAV VP capsid polypeptide of any one of embodiments 2-4, wherein Xaa9 has low mutability.
60. The engineered AAV VP capsid polypeptide of embodiment 59, wherein Xaa9 is selected from P and K.
61. The engineered AAV VP capsid polypeptide of any one of embodiments 2-4, wherein Xaa9 has high average flexibility.
62. The engineered AAV VP capsid polypeptide of embodiment 61, wherein Xaa9 is selected from D, E, P, and S.
63. The engineered AAV VP capsid polypeptide of any one of embodiments 2-4, wherein Xaa9 has high solubility.
64. The engineered AAV VP capsid polypeptide of embodiment 63, wherein Xaa9 is selected from M and V.
65. The engineered AAV VP capsid polypeptide of any one of embodiments 1-64, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 20118-SEQ ID NO: 21117.
66. The engineered AAV VP capsid polypeptide of embodiment 65, wherein the region from the residue corresponding to residue 581 to the residue corresponding to residue 589 inclusive has a sequence of any one of SEQ ID NO: 20118-SEQ ID NO: 21117.
67. The engineered AAV VP capsid polypeptide of any one of embodiments 1-66, wherein tropism for lymph node tissue is measured as a relative accumulation of the rAAV virion in a lymph node tissue as compared to a non-lymph node tissue, wherein the non-lymph node tissue consists collectively of CNS, liver, skeletal muscle, heart, lung, spleen, bone marrow, mammary gland, skin, adrenal gland, thyroid, colon, sciatic nerve, and spinal cord.
68. The engineered AAV VP capsid polypeptide of embodiment 67, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.
69. The engineered AAV VP capsid polypeptide of any one of embodiments 67-68, wherein the higher tissue tropism is a 1.0005-fold to about a 1000-fold increased accumulation in the lymph node tissue as compared to a non-lymph node tissue.
70. The engineered AAV VP capsid polypeptide of embodiment 69, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the lymph node tissue as compared to a non-lymph node tissue.

### Series K Embodiments—Mammary Gland Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a mammary gland tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid.
3. An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a mammary gland tissue as compared to an rAAV virion having a wildtype AAV5 VP capsid polypeptide of SEQ ID NO: 1.
5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 22118-SEQ ID NO: 25117.
6. The engineered AAV VP capsid polypeptide of embodiment 5, wherein the region from the residue corresponding to residue 581 to the residue corresponding to residue 589, inclusive, has a sequence of any one of SEQ ID NO: 22118-SEQ ID NO: 25117.
7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 22118-SEQ ID NO: 25117.
8. The engineered AAV VP capsid polypeptide of embodiment 7, wherein the Xaa1 to Xaa9 region of SEQ ID NO: 2 has a sequence of any one of SEQ ID NO: 22118-SEQ ID NO: 25117.
9. The engineered AAV VP capsid polypeptide of any one of embodiments 2-4, wherein:
Xaa1 is selected from the group consisting of C, K, M, Q, R, and Y; or
Xaa2 is selected from the group consisting of A, F, I, K, S, T, and V; or
Xaa3 is selected from the group consisting of A, F, G, I, K, L, R, T, and Y; or
Xaa4 is selected from the group consisting of A, I, K, Q, R, and T; or
Xaa5 is selected from the group consisting of I, L, M, Q, R, T, V, and Y; or
Xaa6 is selected from the group consisting of H, N, S, and V; or
Xaa7 is selected from the group consisting of A, H, I, N, S and Y; or
Xaa8 is selected from the group consisting of A, C, D, G, H, M, Q, and S; or
Xaa9 is selected from the group consisting of A, E, L, W, and Y; or
any combination thereof.
10. The engineered AAV VP capsid polypeptide of embodiment 9, wherein Xaa1 is selected from C, Q, and R.
11. The engineered AAV VP capsid polypeptide of embodiment 9, wherein Xaa1 is C.
12. The engineered AAV VP capsid polypeptide of embodiment 9, wherein Xaa2 is selected from A, S, and V.
13. The engineered AAV VP capsid polypeptide of embodiment 9, wherein Xaa2 is V.
14. The engineered AAV VP capsid polypeptide of embodiment 9, wherein Xaa3 is selected from F, G, K, R, and Y.
15. The engineered AAV VP capsid polypeptide of embodiment 9, wherein Xaa3 is selected from F, K, and Y.
16. The engineered AAV VP capsid polypeptide of embodiment 9, wherein Xaa3 is F.
17. The engineered AAV VP capsid polypeptide of embodiment 9, wherein Xaa4 is selected from A, I, and R.
18. The engineered AAV VP capsid polypeptide of embodiment 9, wherein Xaa4 is I.
19. The engineered AAV VP capsid polypeptide of embodiment 9, wherein Xaa5 is selected from I, M, and Y.
20. The engineered AAV VP capsid polypeptide of embodiment 9, wherein Xaa5 is Y.
21. The engineered AAV VP capsid polypeptide of embodiment 9, wherein Xaa6 is H.
22. The engineered AAV VP capsid polypeptide of embodiment 9, wherein Xaa7 is N or S.
23. The engineered AAV VP capsid polypeptide of embodiment 9, wherein Xaa7 is N.
24. The engineered AAV VP capsid polypeptide of embodiment 9, wherein Xaa8 is selected from G, M, and Q.
25. The engineered AAV VP capsid polypeptide of embodiment 9, wherein Xaa8 is G.
26. The engineered AAV VP capsid polypeptide of embodiment 9, wherein Xaa9 is selected from A, L, and W.
27. The engineered AAV VP capsid polypeptide of embodiment 9, wherein Xaa9 is A.
28. The engineered AAV VP capsid polypeptide of any one of embodiments 1-27, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 22118-SEQ ID NO: 23117.
29. The engineered AAV VP capsid polypeptide of embodiment 28, wherein the region from residue 581 to residue 589 of SEQ ID NO: 1 has a sequence of any one of SEQ ID NO: 22118-SEQ ID NO: 23117.
30. The engineered AAV VP capsid polypeptide of any one of embodiments 2-4, wherein Xaa1 has low surface accessibility.
31. The engineered AAV VP capsid polypeptide of embodiment 30, wherein Xaa1 is C.
32. The engineered AAV VP capsid polypeptide of any one of embodiments 2-4, wherein Xaa1 has medium mol mass.
33. The engineered AAV VP capsid polypeptide of embodiment 32, wherein Xaa1 is C.
34. The engineered AAV VP capsid polypeptide of any one of embodiments 2-4, wherein Xaa2 has high surface accessibility.
35. The engineered AAV VP capsid polypeptide of embodiment 34, wherein Xaa2 is selected from D, N, and Q.
36. The engineered AAV VP capsid polypeptide of any one of embodiments 2-4, wherein Xaa2 has low hydropathy.
37. The engineered AAV VP capsid polypeptide of embodiment 36, wherein Xaa2 is selected from D, E, R, K, H, N, and Q.
38. The engineered AAV VP capsid polypeptide of any one of embodiments 2-4, wherein Xaa3 has high average flexibility.
39. The engineered AAV VP capsid polypeptide of embodiment 38, wherein Xaa3 is selected from D, E, R, P, G, and S.
40. The engineered AAV VP capsid polypeptide of any one of embodiments 2-4, wherein Xaa3 has medium mutability.
41. The engineered AAV VP capsid polypeptide of embodiment 40, wherein Xaa3 is selected from R and H.
42. The engineered AAV VP capsid polypeptide of any one of embodiments 2-4, wherein Xaa4 has high mutability.
43. The engineered AAV VP capsid polypeptide of embodiment 42, wherein Xaa4 is selected from M, I, Q, and T.
44. The engineered AAV VP capsid polypeptide of any one of embodiments 2-4, wherein Xaa4 has high solubility.
45. The engineered AAV VP capsid polypeptide of embodiment 44, wherein Xaa4 is selected from W, F, I, and L.
46. The engineered AAV VP capsid polypeptide of any one of embodiments 2-4, wherein Xaa4 has high surface accessibility.
47. The engineered AAV VP capsid polypeptide of embodiment 46, wherein Xaa4 is selected from E, R, and K.
48. The engineered AAV VP capsid polypeptide of any one of embodiments 2-4, wherein Xaa5 has high solubility.
49. The engineered AAV VP capsid polypeptide of embodiment 48, wherein Xaa5 is selected from W, F, I, and L.
50. The engineered AAV VP capsid polypeptide of any one of embodiments 2-4, wherein Xaa5 has low mutability.
51. The engineered AAV VP capsid polypeptide of embodiment 50, wherein Xaa5 is selected from Y, F, and L.
52. The engineered AAV VP capsid polypeptide of any one of embodiments 2-4, wherein Xaa6 has high hydropathy.
53. The engineered AAV VP capsid polypeptide of embodiment 52, wherein Xaa6 is selected from V, I, and L.
54. The engineered AAV VP capsid polypeptide of any one of embodiments 2-4, wherein Xaa6 has medium mol mass.
55. The engineered AAV VP capsid polypeptide of embodiment 54, wherein Xaa6 is selected from D, I, L, and N.
56. The engineered AAV VP capsid polypeptide of any one of embodiments 2-4, wherein Xaa8 has low surface accessibility.
57. The engineered AAV VP capsid polypeptide of embodiment 56, wherein Xaa8 is C.
58. The engineered AAV VP capsid polypeptide of any one of embodiments 2-4, wherein Xaa8 has low mutability.
59. The engineered AAV VP capsid polypeptide of embodiment 58, wherein Xaa8 is selected from C, R, and H.
60. The engineered AAV VP capsid polypeptide of any one of embodiments 2-4, wherein Xaa8 has medium mutability.
61. The engineered AAV VP capsid polypeptide of embodiment 60, wherein Xaa9 is selected from R and H.
62. The engineered AAV VP capsid polypeptide of any one of embodiments 1-61, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 23118-SEQ ID NO: 24117.
63. The engineered AAV VP capsid polypeptide of embodiment 62, wherein the region from the residue corresponding to residue 581 to the residue corresponding to residue 589 inclusive has a sequence of any one of SEQ ID NO: 23118-SEQ ID NO: 24117.
64. The engineered AAV VP capsid polypeptide of any one of embodiments 1-63, wherein tropism for mammary gland tissue is measured as a relative accumulation of the rAAV virion in a mammary gland tissue as compared to a non-mammary gland tissue, wherein the non-mammary gland tissue consists collectively of CNS, liver, skeletal muscle, heart, lung, spleen, lymph node, bone marrow, skin, adrenal gland, thyroid, colon, sciatic nerve, and spinal cord.
65. The engineered AAV VP capsid polypeptide of embodiment 65, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.
66. The engineered AAV VP capsid polypeptide of any one of embodiments 64-65, wherein the higher tissue tropism is a 1.0005-fold to about a 1000-fold increased accumulation in the mammary gland tissue as compared to a non-mammary gland tissue.
67. The engineered AAV VP capsid polypeptide of embodiment 66, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the mammary gland tissue as compared to a non-mammary gland tissue.

### Series L Embodiments—Skeletal or Cardiac Muscle Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a skeletal muscle tissue or a cardiac muscle tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid. 3. An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a skeletal muscle tissue or a cardiac muscle tissue as compared to an rAAV virion having a wildtype AAV5 VP capsid polypeptide of SEQ ID NO: 1.
5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 25118-SEQ ID NO: 26117.
6. The engineered AAV VP capsid polypeptide of embodiment 5, wherein the region from the residue corresponding to residue 581 to the residue corresponding to residue 589, inclusive, has a sequence of any one of SEQ ID NO: 25118-SEQ ID NO: 26117.
7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 25118-SEQ ID NO: 26117.
8. The engineered AAV VP capsid polypeptide of embodiment 7, wherein the Xaa1 to Xaa9 region of SEQ ID NO: 2 has a sequence of any one of SEQ ID NO: 25118-SEQ ID NO: 26117.
9. The engineered AAV VP capsid polypeptide of any one of embodiments 2-4, wherein Xaa1 has low solubility.
10. The engineered AAV VP capsid polypeptide of embodiment 9, wherein Xaa1 is selected from D, E, R, K, P, N, and Q.
11. The engineered AAV VP capsid polypeptide of any one of embodiments 2-4, wherein Xaa1 has low hydropathy.
12. The engineered AAV VP capsid polypeptide of embodiment 11, wherein Xaa1 is selected from D, E, R, K, Q, N, Y, P.
13. The engineered AAV VP capsid polypeptide of any one of embodiments 2-4, wherein Xaa1 has high surface accessibility.
14. The engineered AAV VP capsid polypeptide of embodiment 13, wherein Xaa1 is selected from E, R, and K.
15. The engineered AAV VP capsid polypeptide of any one of embodiments 2-4, wherein Xaa2 has high hydropathy.
16. The engineered AAV VP capsid polypeptide of embodiment 15, wherein Xaa2 is selected from V, I, F, L, and C.
17. The engineered AAV VP capsid polypeptide of any one of embodiments 2-4, wherein Xaa2 has low mutability.
18. The engineered AAV VP capsid polypeptide of embodiment 17, wherein Xaa2 is selected from R, V, I, H, and C.
19. The engineered AAV VP capsid polypeptide of any one of embodiments 2-4, wherein Xaa2 has medium volume.
20. The engineered AAV VP capsid polypeptide of embodiment 19, wherein Xaa2 is selected from E, V, and Q.
21. The engineered AAV VP capsid polypeptide of any one of embodiments 2-4, wherein Xaa3 has low solubility.
22. The engineered AAV VP capsid polypeptide of embodiment 21, wherein Xaa3 is selected from D, R, and Q.
23. The engineered AAV VP capsid polypeptide of any one of embodiments 2-4, wherein Xaa4 has low solubility.
24. The engineered AAV VP capsid polypeptide of embodiment 23, wherein Xaa4 is selected from D, E, P, and N.
25. The engineered AAV VP capsid polypeptide of any one of embodiments 2-4, wherein Xaa4 has low charge.
26. The engineered AAV VP capsid polypeptide of embodiment 25, wherein Xaa4 is selected from D and E.
27. The engineered AAV VP capsid polypeptide of any one of embodiments 2-4, wherein Xaa5 has low amino acid solubility.
28. The engineered AAV VP capsid polypeptide of embodiment 27, wherein Xaa5 is selected from D, E, R, K, N, Q.
29. The engineered AAV VP capsid polypeptide of any one of embodiments 2-4, wherein Xaa8 has low solubility.
30. The engineered AAV VP capsid polypeptide of embodiment 29, wherein Xaa8 is selected from D, E, K, P, and N.
31. The engineered AAV VP capsid polypeptide of any one of embodiments 2-4, wherein Xaa8 has high flexibility index.
32. The engineered AAV VP capsid polypeptide of embodiment 31, wherein Xaa8 is selected from Q, S, P, E, and D.
33. The engineered AAV VP capsid polypeptide of any one of embodiments 2-4, wherein Xaa8 has high surface accessibility.
34. The engineered AAV VP capsid polypeptide of embodiment 33, wherein Xaa8 is selected from S, D, P, N, E, R, and K.
35. The engineered AAV VP capsid polypeptide of any one of embodiments 1-34, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 25118-SEQ ID NO: 26117.
36. The engineered AAV VP capsid polypeptide of embodiment 35, wherein the region from the residue corresponding to residue 581 to the residue corresponding to residue 589 inclusive has a sequence of any one of SEQ ID NO: 25118-SEQ ID NO: 26117.
37. The engineered AAV VP capsid polypeptide of any one of embodiments 1-36, wherein tropism for skeletal muscle tissue or cardiac muscle tissue is measured as a relative accumulation of the rAAV virion in a skeletal muscle tissue or a cardiac muscle tissue as compared to a non-skeletal muscle tissue or a non-cardiac muscle tissue, wherein the non-skeletal muscle tissue or a non-cardiac muscle tissue consists collectively of CNS, liver, lung, spleen, lymph node, bone marrow, mammary gland, skin, adrenal gland, thyroid, colon, sciatic nerve, and spinal cord.
38. The engineered AAV VP capsid polypeptide of embodiment 37, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.
39. The engineered AAV VP capsid polypeptide of any one of embodiments 37-38, wherein the higher tissue tropism is a 1.0005-fold to about a 1000-fold increased accumulation in the skeletal muscle tissue or a cardiac muscle tissue as compared to a non-skeletal muscle tissue or a non-cardiac muscle tissue.
40. The engineered AAV VP capsid polypeptide of embodiment 39, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the skeletal muscle tissue or a cardiac muscle tissue as compared to a non-skeletal muscle tissue or a non-cardiac muscle tissue.

### Series M Embodiments—Sciatic Nerve Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a sciatic nerve tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid. 3.An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a sciatic nerve tissue as compared to an rAAV virion having a wildtype AAV5 VP capsid polypeptide of SEQ ID NO: 1. 5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 26118-SEQ ID NO: 28990.
6. The engineered AAV VP capsid polypeptide of embodiment 5, wherein the region from the residue corresponding to residue 581 to the residue corresponding to residue 589, inclusive, has a sequence of any one of SEQ ID NO: 26118-SEQ ID NO: 28990.
7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 26118-SEQ ID NO: 28990.
8. The engineered AAV VP capsid polypeptide of embodiment 7, wherein the Xaa1 to Xaa9 region of SEQ ID NO: 2 has a sequence of any one of SEQ ID NO: 26118-SEQ ID NO: 28990.
9. The engineered AAV VP capsid polypeptide of any one of embodiments 2-4, wherein:
   Xaa1 is selected from the group consisting of C, G, K, M, Q, R, and Y; or
   Xaa2 is selected from the group consisting of A, C, F, I, Q, T, and V; or
   Xaa3 is selected from the group consisting of A, F, I, M, R, S, and T; or
   Xaa4 is selected from the group consisting of E, N, T, Q, and V; or
   Xaa5 is selected from the group consisting of F, H, Q, S, V, and Y; or
   Xaa6 is selected from the group consisting of K, M, N, Q, S, and V; or
   Xaa7 is selected from the group consisting of K, M, Q, R, and T; or
   Xaa8 is selected from the group consisting of A, G, H, Q, S, and V; or
   Xaa9 is selected from the group consisting of C, E, I, K, and R; or
   any combination thereof.
10. The engineered AAV VP capsid polypeptide of embodiment 9, wherein Xaa1 is selected from C, R, and Q.
11. The engineered AAV VP capsid polypeptide of embodiment 9, wherein Xaa1 is C.
12. The engineered AAV VP capsid polypeptide of embodiment 9, wherein Xaa2 is selected from A, C, and I.
13. The engineered AAV VP capsid polypeptide of embodiment 9, wherein Xaa2 is A.
14. The engineered AAV VP capsid polypeptide of embodiment 9, wherein Xaa3 is selected from F, M, R, and S.
15. The engineered AAV VP capsid polypeptide of embodiment 9, wherein Xaa3 is R.
16. The engineered AAV VP capsid polypeptide of embodiment 9, wherein Xaa4 is selected from E, T, and V.
17. The engineered AAV VP capsid polypeptide of embodiment 9, wherein Xaa4 is T.
18. The engineered AAV VP capsid polypeptide of embodiment 9, wherein Xaa5 is selected from F, V, and Y.
19. The engineered AAV VP capsid polypeptide of embodiment 9, wherein Xaa5 is V.
20. The engineered AAV VP capsid polypeptide of embodiment 9, wherein Xaa6 is selected from M, N, and S.
21. The engineered AAV VP capsid polypeptide of embodiment 9, wherein Xaa6 is N.
22. The engineered AAV VP capsid polypeptide of embodiment 9, wherein Xaa7 is selected from M, Q, and T.
23. The engineered AAV VP capsid polypeptide of embodiment 9, wherein Xaa7 is M.
24. The engineered AAV VP capsid polypeptide of embodiment 9, wherein Xaa8 is selected from H and S.
25. The engineered AAV VP capsid polypeptide of embodiment 9, wherein Xaa8 is H.
26. The engineered AAV VP capsid polypeptide of embodiment 9, wherein Xaa9 is selected from C, I, and K.
27. The engineered AAV VP capsid polypeptide of embodiment 9, wherein Xaa9 is I.
28. The engineered AAV VP capsid polypeptide of any one of embodiments 1-27, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 26118-SEQ ID NO: 26990.
29. The engineered AAV VP capsid polypeptide of embodiment 28, wherein the region from residue 581 to residue 589 of SEQ ID NO: 1 has a sequence of any one of SEQ ID NO: 26118-SEQ ID NO: 26990.
30. The engineered AAV VP capsid polypeptide of any one of embodiments 2-4, wherein Xaa1 has high average flexibility.
31. The engineered AAV VP capsid polypeptide of embodiment 30, wherein Xaa1 is selected from G and R.
32. The engineered AAV VP capsid polypeptide of any one of embodiments 2-4, wherein Xaa1 has low solubility.
33. The engineered AAV VP capsid polypeptide of embodiment 32, wherein Xaa1 is selected from R and Q.
34. The engineered AAV VP capsid polypeptide of any one of embodiments 2-4, wherein Xaa1 has low mutability.
35. The engineered AAV VP capsid polypeptide of embodiment 34, wherein Xaa1 is selected from C, L, F, Y, R, K, P, and H.
36. The engineered AAV VP capsid polypeptide of any one of embodiments 2-4, wherein Xaa1 has high volume.
37. The engineered AAV VP capsid polypeptide of embodiment 36, wherein Xaa1 is selected from Y and F.
38. The engineered AAV VP capsid polypeptide of any one of embodiments 2-4, wherein Xaa2 has high surface accessibility.
39. The engineered AAV VP capsid polypeptide of embodiment 38, wherein Xaa2 is selected from E, R, and K.
40. The engineered AAV VP capsid polypeptide of any one of embodiments 2-4, wherein Xaa3 has medium mutability.
41. The engineered AAV VP capsid polypeptide of embodiment 40, wherein Xaa3 is selected from H and R.
42. The engineered AAV VP capsid polypeptide of any one of embodiments 2-4, wherein Xaa3 has medium average flexibility.
43. The engineered AAV VP capsid polypeptide of embodiment 42, wherein Xaa3 is selected from V and Y.
44. The engineered AAV VP capsid polypeptide of any one of embodiments 2-4, wherein Xaa4 has high mutability.
45. The engineered AAV VP capsid polypeptide of embodiment 44, wherein Xaa4 is N.
46. The engineered AAV VP capsid polypeptide of any one of embodiments 2-4, wherein Xaa4 has high average flexibility.
47. The engineered AAV VP capsid polypeptide of embodiment 46, wherein Xaa4 is selected from I, N, G, and R.
48. The engineered AAV VP capsid polypeptide of any one of embodiments 2-4, wherein Xaa4 has low solubility.
49. The engineered AAV VP capsid polypeptide of embodiment 48, wherein Xaa4 is N.
50. The engineered AAV VP capsid polypeptide of any one of embodiments 2-4, wherein Xaa6 has low mutability.
51. The engineered AAV VP capsid polypeptide of embodiment 50, wherein Xaa6 is selected from C, L, F, and Y.
52. The engineered AAV VP capsid polypeptide of any one of embodiments 2-4, wherein Xaa6 has high volume.
53. The engineered AAV VP capsid polypeptide of embodiment 52, wherein Xaa6 is selected from K, M, I, and L.
54. The engineered AAV VP capsid polypeptide of any one of embodiments 2-4, wherein Xaa7 has low mutability.
55. The engineered AAV VP capsid polypeptide of embodiment 54, wherein Xaa7 is selected from L, F, and Y.
56. The engineered AAV VP capsid polypeptide of any one of embodiments 2-4, wherein Xaa7 has medium mol mass.
57. The engineered AAV VP capsid polypeptide of embodiment 56, wherein Xaa7 is selected from D, I, L, and N.
58. The engineered AAV VP capsid polypeptide of any one of embodiments 2-4, wherein Xaa8 has high surface accessibility.
59. The engineered AAV VP capsid polypeptide of embodiment 58, wherein Xaa8 is selected from S, Y, T, D, P, H, and N.
60. The engineered AAV VP capsid polypeptide of any one of embodiments 2-4, wherein Xaa9 has low mutability.
61. The engineered AAV VP capsid polypeptide of embodiment 60, wherein Xaa9 is selected from C, H, and R.
62. The engineered AAV VP capsid polypeptide of any one of embodiments 2-4, wherein Xaa9 has medium solubility.
63. The engineered AAV VP capsid polypeptide of embodiment 62, wherein Xaa9 is selected from Q, T, and C.
64. The engineered AAV VP capsid polypeptide of any one of embodiments 2-4, wherein Xaa9 has low surface accessibility.
65. The engineered AAV VP capsid polypeptide of embodiment 64, wherein Xaa9 is C.
66. The engineered AAV VP capsid polypeptide of any one of embodiments 1-65, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 26991-SEQ ID NO: 27990.
67. The engineered AAV VP capsid polypeptide of embodiment 66, wherein the region from the residue corresponding to residue 581 to the residue corresponding to residue 589 inclusive has a sequence of any one of SEQ ID NO: 26991-SEQ ID NO: 27990.
68. The engineered AAV VP capsid polypeptide of any one of embodiments 1-67, wherein tropism for sciatic nerve tissue is measured as a relative accumulation of the rAAV virion in a sciatic nerve tissue as compared to a non-sciatic nerve tissue, wherein the non-sciatic nerve tissue consists collectively of CNS, liver, skeletal muscle, heart, lung, spleen, lymph node, bone marrow, mammary gland, skin, adrenal gland, thyroid, colon, and spinal cord.
69. The engineered AAV VP capsid polypeptide of any one of embodiments 68, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.
70. The engineered AAV VP capsid polypeptide of any one of embodiments 68-69, wherein the higher tissue tropism is a 1.0005-fold to about a 1000-fold increased accumulation in the sciatic nerve tissue as compared to a non-sciatic nerve tissue.
71. The engineered AAV VP capsid polypeptide of embodiment 70, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the sciatic nerve tissue as compared to a non-sciatic nerve tissue.

### Series N Embodiments—Skeletal Muscle Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a skeletal muscle tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid. 3.An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a skeletal muscle tissue as compared to an rAAV virion having a wildtype AAV5 VP capsid polypeptide of SEQ ID NO: 1. 5. The engineered AAV VP capsid polypeptide of any one of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 28991-SEQ ID NO: 31990. 6. The engineered AAV VP capsid polypeptide of embodiment 5, wherein the region from the residue corresponding to residue 581 to the residue corresponding to residue 589, inclusive, has a sequence of any one of SEQ ID NO: 28991-SEQ ID NO: 31990. 7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 28991-SEQ ID NO: 31990.
8. The engineered AAV VP capsid polypeptide of embodiment 7, wherein the Xaa1 to Xaa9 region of SEQ ID NO: 2 has a sequence of any one of SEQ ID NO: 28991-SEQ ID NO: 31990.
9. The engineered AAV VP capsid polypeptide of any one of embodiments X-Y, wherein:
   Xaa1 is selected from the group consisting of A, E, H, M, P, Q, and S; or
   Xaa2 is selected from the group consisting of F, H, I, T, and V; or
   Xaa3 is selected from the group consisting of A, G, I, K, M, Q, R, S, T, and V; or
   Xaa4 is selected from the group consisting of D, E, G, P, and S; or
   Xaa5 is selected from the group consisting of H, L, M, P, and V; or
   Xaa6 is selected from the group consisting of E, H, N, and P; or
   Xaa7 is selected from the group consisting of A, H, N, Q and T; or
   Xaa8 is selected from the group consisting of I, K, M, P, and W; or
   Xaa9 is selected from the group consisting of A, I, M, P, and V; or
   any combination thereof.
10. The engineered AAV VP capsid polypeptide of embodiment 9, wherein Xaa1 is selected from P and Q.
11. The engineered AAV VP capsid polypeptide of embodiment 9, wherein Xaa1 is Q.
12. The engineered AAV VP capsid polypeptide of embodiment 9, wherein Xaa2 is selected from T and V.
13. The engineered AAV VP capsid polypeptide of embodiment 9, wherein Xaa2 is V.
14. The engineered AAV VP capsid polypeptide of embodiment 9, wherein Xaa3 is selected from A, L, P, R, and T.
15. The engineered AAV VP capsid polypeptide of embodiment 9, wherein Xaa3 is selected from L, P, and T.
16. The engineered AAV VP capsid polypeptide of embodiment 9, wherein Xaa3 is P.
17. The engineered AAV VP capsid polypeptide of embodiment 9, wherein Xaa4 is selected from D, E, and S.
18. The engineered AAV VP capsid polypeptide of embodiment 9, wherein Xaa4 is E.
19. The engineered AAV VP capsid polypeptide of embodiment 9, wherein Xaa5 is selected from L, M, and V.
20. The engineered AAV VP capsid polypeptide of embodiment 9, wherein Xaa5 is L.
21. The engineered AAV VP capsid polypeptide of embodiment 9, wherein Xaa6 is P.
22. The engineered AAV VP capsid polypeptide of embodiment 9, wherein Xaa7 is H.
23. The engineered AAV VP capsid polypeptide of embodiment 9, wherein Xaa8 is selected from I, P, and W.
24. The engineered AAV VP capsid polypeptide of embodiment 9, wherein Xaa8 is P.
25. The engineered AAV VP capsid polypeptide of embodiment 9, wherein Xaa9 is selected from A, M, and P.
26. The engineered AAV VP capsid polypeptide of embodiment 9, wherein Xaa9 is M.
27. The engineered AAV VP capsid polypeptide of any one of embodiments 1-26, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 28991-SEQ ID NO: 29990.
28. The engineered AAV VP capsid polypeptide of embodiment 27, wherein the region from residue 581 to residue 589 of SEQ ID NO: 1 has a sequence of any one of SEQ ID NO: 28991-SEQ ID NO: 29990.
29. The engineered AAV VP capsid polypeptide of any one of embodiments 2-4, wherein Xaa1 has high average flexibility.
30. The engineered AAV VP capsid polypeptide of embodiment 29, wherein Xaa1 is selected from G and R.
31. The engineered AAV VP capsid polypeptide of any one of embodiments 2-4, wherein Xaa1 has low average flexibility.
32. The engineered AAV VP capsid polypeptide of embodiment 31, wherein Xaa1 is selected from W, M, F, and H.
33. The engineered AAV VP capsid polypeptide of any one of embodiments 2-4, wherein Xaa1 has high mol mass.
34. The engineered AAV VP capsid polypeptide of embodiment 33, wherein Xaa1 is selected from R, F, and W.
35. The engineered AAV VP capsid polypeptide of any one of embodiments 2-4, wherein Xaa2 has low hydropathy.
36. The engineered AAV VP capsid polypeptide of embodiment 35, wherein Xaa2 is selected from K and R.
37. The engineered AAV VP capsid polypeptide of any one of embodiments 2-4, wherein Xaa2 has low mutability.
38. The engineered AAV VP capsid polypeptide of embodiment 37, wherein Xaa2 is selected from C, R, and H.
39. The engineered AAV VP capsid polypeptide of any one of embodiments 2-4, wherein Xaa2 has high average flexibility.
40. The engineered AAV VP capsid polypeptide of embodiment 39, wherein Xaa2 is selected from G and R.
41. The engineered AAV VP capsid polypeptide of any one of embodiments 2-4, wherein Xaa3 has high average flexibility.
42. The engineered AAV VP capsid polypeptide of embodiment 41, wherein Xaa3 is selected from G and R.
43. The engineered AAV VP capsid polypeptide of any one of embodiments 2-4, wherein Xaa4 has high hydrophilicity.
44. The engineered AAV VP capsid polypeptide of embodiment 43, wherein Xaa4 is selected from D, E, R, K, and N.
45. The engineered AAV VP capsid polypeptide of any one of embodiments 2-4, wherein Xaa4 has low mutability.
46. The engineered AAV VP capsid polypeptide of embodiment 45, wherein Xaa4 is selected from C, R, and H.
47. The engineered AAV VP capsid polypeptide of any one of embodiments 2-4, wherein Xaa5 has low mol mass.
48. The engineered AAV VP capsid polypeptide of embodiment 47, wherein Xaa5 is A.
49. The engineered AAV VP capsid polypeptide of any one of embodiments 2-4, wherein Xaa5 has low average flexibility.
50. The engineered AAV VP capsid polypeptide of embodiment 49, wherein Xaa5 is selected from A and L.
51. The engineered AAV VP capsid polypeptide of any one of embodiments 2-4, wherein Xaa5 has high mutability.
52. The engineered AAV VP capsid polypeptide of embodiment 51, wherein Xaa5 is selected from D, A, and E.
53. The engineered AAV VP capsid polypeptide of any one of embodiments 2-4, wherein Xaa6 has low average flexibility.
54. The engineered AAV VP capsid polypeptide of embodiment 53, wherein Xaa6 is selected from W, M, and F.
55. The engineered AAV VP capsid polypeptide of any one of embodiments 2-4, wherein Xaa6 has low mutability.
56. The engineered AAV VP capsid polypeptide of embodiment 55, wherein Xaa6 is C.
57. The engineered AAV VP capsid polypeptide of any one of embodiments 2-4, wherein Xaa6 has high mol mass.
58. The engineered AAV VP capsid polypeptide of embodiment 57, wherein Xaa6 is W.
59. The engineered AAV VP capsid polypeptide of any one of embodiments 2-4, wherein Xaa7 has low goldman engelman steitz.
60. The engineered AAV VP capsid polypeptide of embodiment 59, wherein Xaa7 is R.
61. The engineered AAV VP capsid polypeptide of any one of embodiments 2-4, wherein Xaa7 has high average flexibility.
62. The engineered AAV VP capsid polypeptide of embodiment 61, wherein Xaa7 is selected from D, R, P, G, and S.
63. The engineered AAV VP capsid polypeptide of any one of embodiments 2-4, wherein Xaa7 has high mutability.
64. The engineered AAV VP capsid polypeptide of embodiment 63, wherein Xaa7 is selected from R, H, and N.
65. The engineered AAV VP capsid polypeptide of any one of embodiments 2-4, wherein Xaa7 has low solubility.
66. The engineered AAV VP capsid polypeptide of embodiment 65, wherein Xaa7 is selected from R and Q.
67. The engineered AAV VP capsid polypeptide of any one of embodiments 2-4, wherein Xaa8 has high hydrophilicity.
68. The engineered AAV VP capsid polypeptide of embodiment 67, wherein Xaa8 is selected from D, E, R, K, and N.
69. The engineered AAV VP capsid polypeptide of any one of embodiments 2-4, wherein Xaa9 has low mutability.
70. The engineered AAV VP capsid polypeptide of embodiment 69, wherein Xaa9 is selected from Y, F, and L.
71. The engineered AAV VP capsid polypeptide of any one of embodiments 1-70, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 29991-SEQ ID NO: 30990.
72. The engineered AAV VP capsid polypeptide of embodiment 71, wherein the region from the residue corresponding to residue 581 to the residue corresponding to residue 589 inclusive has a sequence of any one of SEQ ID NO: 29991-SEQ ID NO: 30990.
73. The engineered AAV VP capsid polypeptide of any one of embodiments 1-72, wherein tropism for skeletal muscle tissue is measured as a relative accumulation of the rAAV virion in a skeletal muscle tissue as compared to a non-skeletal muscle tissue, wherein the non-skeletal muscle tissue consists collectively of CNS, liver, heart, lung, spleen, lymph node, bone marrow, mammary gland, skin, adrenal gland, thyroid, colon, sciatic nerve, and spinal cord.
74. The engineered AAV VP capsid polypeptide of embodiment 73, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.
75. The engineered AAV VP capsid polypeptide of any one of embodiments 73 and 74, wherein the higher tissue tropism is a 1.0005-fold to about a 1000-fold increased accumulation in the skeletal muscle tissue as compared to a non-skeletal muscle tissue.
76. The engineered AAV VP capsid polypeptide of embodiment 75, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the skeletal muscle tissue as compared to a non-skeletal muscle tissue.

### Series O Embodiments—Skin Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a skin tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid. 3.An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a skin tissue as compared to an rAAV virion having a wildtype AAV5 VP capsid polypeptide of SEQ ID NO: 1. 5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 31991-SEQ ID NO: 34990.
6. The engineered AAV VP capsid polypeptide of embodiment 5, wherein the region from the residue corresponding to residue 581 to the residue corresponding to residue 589, inclusive, has a sequence of any one of SEQ ID NO: 31991-SEQ ID NO: 34990.
7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 31991-SEQ ID NO: 34990.
8. The engineered AAV VP capsid polypeptide of embodiment 7, wherein the Xaa1 to Xaa9 region of SEQ ID NO: 2 has a sequence of any one of SEQ ID NO: 31991-SEQ ID NO: 34990
9. The engineered AAV VP capsid polypeptide of any one of embodiments 2-4, wherein:
   Xaa1 is selected from the group consisting of A, C, K, Q, R, and T; or
   Xaa2 is selected from the group consisting of A, C, I, S, T, and V; or
   Xaa3 is selected from the group consisting of A, C, F, G, M, Q, S, and V; or
   Xaa4 is selected from the group consisting of C, K, L, P, R, and W; or
   Xaa5 is selected from the group consisting of F, H, I, M, V, and Y; or
   Xaa6 is selected from the group consisting of F, H, I, M, N, Q, and S; or
   Xaa7 is selected from the group consisting of A, H, K, M, N, R, and V; or
   Xaa8 is selected from the group consisting of A, F, G, H, S, and Y; or
   Xaa9 is selected from the group consisting of A, E, G, P, Q, R, and S; or
   any combination thereof.
10. The engineered AAV VP capsid polypeptide of embodiment 9, wherein Xaa1 is selected from C, K, and R.
11. The engineered AAV VP capsid polypeptide of embodiment 9, wherein Xaa1 is C.
12. The engineered AAV VP capsid polypeptide of embodiment 9, wherein Xaa2 is selected from A, S, T, and V.
13. The engineered AAV VP capsid polypeptide of embodiment 9, wherein Xaa2 is V.
14. The engineered AAV VP capsid polypeptide of embodiment 9, wherein Xaa3 is selected from A, C, F, M, and Q.
15. The engineered AAV VP capsid polypeptide of embodiment 9, wherein Xaa3 is C.
16. The engineered AAV VP capsid polypeptide of embodiment 9, wherein Xaa4 is selected from L, P, and R.
17. The engineered AAV VP capsid polypeptide of embodiment 9, wherein Xaa4 is R.
18. The engineered AAV VP capsid polypeptide of embodiment 9, wherein Xaa5 is selected from M, V, and Y.
19. The engineered AAV VP capsid polypeptide of embodiment 9, wherein Xaa5 is Y.
20. The engineered AAV VP capsid polypeptide of embodiment 9, wherein Xaa6 is selected from M, N, and Q.
21. The engineered AAV VP capsid polypeptide of embodiment 9, wherein Xaa6 is N.
22. The engineered AAV VP capsid polypeptide of embodiment 9, wherein Xaa7 is A, H, K, or R.
23. The engineered AAV VP capsid polypeptide of embodiment 9, wherein Xaa7 is K.
24. The engineered AAV VP capsid polypeptide of embodiment 9, wherein Xaa8 is selected from A, F, and S.
25. The engineered AAV VP capsid polypeptide of embodiment 9, wherein Xaa8 is S.
26. The engineered AAV VP capsid polypeptide of embodiment 9, wherein Xaa9 is selected from A, Q, and S.
27. The engineered AAV VP capsid polypeptide of embodiment 9, wherein Xaa9 is A.
28. The engineered AAV VP capsid polypeptide of any one of embodiments 1-27, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 31991-SEQ ID NO: 32990.
29. The engineered AAV VP capsid polypeptide of embodiment 28 wherein the region from residue 581 to residue 589 of SEQ ID NO: 1 has a sequence of any one of SEQ ID NO: 31991-SEQ ID NO: 32990.
30. The engineered AAV VP capsid polypeptide of any one of embodiments 2-4, wherein Xaa1 has low surface accessibility.
31. The engineered AAV VP capsid polypeptide of embodiment 30, wherein Xaa1 is C.
32. The engineered AAV VP capsid polypeptide of any one of embodiments 2-4, wherein Xaa1 has low volume.
33. The engineered AAV VP capsid polypeptide of embodiment 32, wherein Xaa1 is C.
34. The engineered AAV VP capsid polypeptide of any one of embodiments 2-4, wherein Xaa1 has low mutability.
35. The engineered AAV VP capsid polypeptide of embodiment 34, wherein Xaa1 is C.
36. The engineered AAV VP capsid polypeptide of any one of embodiments 2-4, wherein Xaa2 has high surface accessibility.
37. The engineered AAV VP capsid polypeptide of embodiment 36, wherein Xaa2 is selected from R and K.
38. The engineered AAV VP capsid polypeptide of any one of embodiments 2-4, wherein Xaa2 has high average flexibility.
39. The engineered AAV VP capsid polypeptide of embodiment 38, wherein Xaa2 is selected from K, I, and N.
40. The engineered AAV VP capsid polypeptide of any one of embodiments 2-4, wherein Xaa2 has low mutability.
41. The engineered AAV VP capsid polypeptide of embodiment 40, wherein Xaa2 is selected from P and K.
42. The engineered AAV VP capsid polypeptide of any one of embodiments 2-4, wherein Xaa3 has high hydropathy.
43. The engineered AAV VP capsid polypeptide of embodiment 42, wherein Xaa3 is selected from I and V.
44. The engineered AAV VP capsid polypeptide of any one of embodiments 2-4, wherein Xaa4 has low mutability.
45. The engineered AAV VP capsid polypeptide of embodiment 44, wherein Xaa4 is selected from L, F, and Y.
46. The engineered AAV VP capsid polypeptide of any one of embodiments 2-4, wherein Xaa4 has low average flexibility.
47. The engineered AAV VP capsid polypeptide of embodiment 46, wherein Xaa4 is selected from W, H, F, and M.
48. The engineered AAV VP capsid polypeptide of any one of embodiments 2-4, wherein Xaa5 has high average flexibility.
49. The engineered AAV VP capsid polypeptide of embodiment 48, wherein Xaa5 is selected from G, R, K, I, and N.
50. The engineered AAV VP capsid polypeptide of any one of embodiments 2-4, wherein Xaa6 has high average flexibility.
51. The engineered AAV VP capsid polypeptide of embodiment 50, wherein Xaa6 is selected from G, R, K, I, and N.
52. The engineered AAV VP capsid polypeptide of any one of embodiments 2-4, wherein Xaa8 has high surface accessibility.
53. The engineered AAV VP capsid polypeptide of embodiment 52, wherein Xaa8 is selected from M, G, and F.
54. The engineered AAV VP capsid polypeptide of any one of embodiments 2-4, wherein Xaa8 has low average flexibility.
55. The engineered AAV VP capsid polypeptide of embodiment 54, wherein Xaa8 is selected from H, F, M, and W.
56. The engineered AAV VP capsid polypeptide of any one of embodiments 2-4, wherein Xaa8 has low mutability.
57. The engineered AAV VP capsid polypeptide of embodiment 56, wherein Xaa8 is selected from L, F, and Y.
58. The engineered AAV VP capsid polypeptide of any one of embodiments 2-4, wherein Xaa9 has high average flexibility.
59. The engineered AAV VP capsid polypeptide of embodiment 58, wherein Xaa9 is selected from D, E, R, K, P, and G.
60. The engineered AAV VP capsid polypeptide of any one of embodiments 2-4, wherein Xaa9 has high mutability.
61. The engineered AAV VP capsid polypeptide of embodiment 60, wherein Xaa9 is selected from D, E, R, V, A, and H.
62. The engineered AAV VP capsid polypeptide of any one of embodiments 1-61, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 32991-SEQ ID NO: 33990.
63. The engineered AAV VP capsid polypeptide of embodiment 62, wherein the region from the residue corresponding to residue 581 to the residue corresponding to residue 589 inclusive has a sequence of any one of SEQ ID NO: 32991-SEQ ID NO: 33990.
64. The engineered AAV VP capsid polypeptide of any one of embodiments 1-61, wherein tropism for skin tissue is measured as a relative accumulation of the rAAV virion in a skin tissue as compared to a non-skin tissue, wherein the non-skin tissue consists collectively of CNS, liver, skeletal muscle, heart, lung, spleen, lymph node, bone marrow, mammary gland, adrenal gland, thyroid, colon, sciatic nerve, and spinal cord.
65. The engineered AAV VP capsid polypeptide of embodiment 64, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.
66. The engineered AAV VP capsid polypeptide of any one of embodiments 64-65, wherein the higher tissue tropism is a 1.0005-fold to about a 1000-fold increased accumulation in the skin tissue as compared to a non-skin tissue.
67. The engineered AAV VP capsid polypeptide of embodiment 66, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the skin tissue as compared to a non-skin tissue.

### Series P Embodiments—Spinal Cord Tropic Capsids

1. An engineered adeno-associated virus (AAV) viral protein (VP) capsid polypeptide having an amino acid sequence at least 70% identical to SEQ ID NO: 1,
   wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
   wherein the engineered AAV VP capsid polypeptide is capable of assembling into a recombinant AAV virion (rAAV),
   wherein the at least one mutation confers higher tropism for a spinal cord tissue on the rAAV as compared to an rAAV virion having a wildtype AAV5 VP capsid polypeptide of SEQ ID NO: 1, and
   wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid. 3. An engineered adeno-associated virus (AAV) viral protein (VP) capsid polypeptide having an amino acid sequence of SEQ ID NO: 2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
   wherein the engineered AAV VP capsid polypeptide is capable of assembling into a recombinant AAV virion (rAAV), and
   wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a spinal cord tissue as compared to an rAAV virion having a wildtype AAV5 VP capsid polypeptide of SEQ ID NO: 1. 5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 34991-SEQ ID NO: 37437. 6. The engineered AAV VP capsid polypeptide of embodiment 5, wherein the region from the residue corresponding to residue 581 to the residue corresponding to residue 589, inclusive, has a sequence of any one of SEQ ID NO: 34991-SEQ ID NO: 37437. 7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 34991-SEQ ID NO: 37437.
8. The engineered AAV VP capsid polypeptide of embodiment 7, wherein the Xaa1 to Xaa9 region of SEQ ID NO: 2 has a sequence of any one of SEQ ID NO: 34991-SEQ ID NO: 37437.
9. The engineered AAV VP capsid polypeptide of any one of embodiments X-Y, wherein:
   Xaa1 is selected from the group consisting of A, C, K, Q, R, S, and W; or
   Xaa2 is selected from the group consisting of H, I, K, L, T, V, and W; or
   Xaa3 is selected from the group consisting of C, F, G, H, I, K, N, and R; or
   Xaa4 is selected from the group consisting of I, M, Q, S, and V; or
   Xaa5 is selected from the group consisting of H, K, Q, T, W, and Y; or
   Xaa6 is selected from the group consisting of H, L, N, Q, R, W, and Y; or
   Xaa7 is selected from the group consisting of D, H, P, Q, and R; or
   Xaa8 is selected from the group consisting of D, F, L, S, T, and Y; or
   Xaa9 is selected from the group consisting of C, I, N, P, R, S, and Y; or
   any combination thereof.
10. The engineered AAV VP capsid polypeptide of embodiment 9, wherein Xaa1 is selected from K, R, and W.
11. The engineered AAV VP capsid polypeptide of embodiment 9, wherein Xaa1 is K.
12. The engineered AAV VP capsid polypeptide of embodiment 9, wherein Xaa2 is selected from H, I, and T.
13. The engineered AAV VP capsid polypeptide of embodiment 9, wherein Xaa2 is I.
14. The engineered AAV VP capsid polypeptide of embodiment 9, wherein Xaa3 is selected from F, I, and R.
15. The engineered AAV VP capsid polypeptide of embodiment 9, wherein Xaa3 is I.
16. The engineered AAV VP capsid polypeptide of embodiment 9, wherein Xaa4 is selected from I, M, and V.
17. The engineered AAV VP capsid polypeptide of embodiment 9, wherein Xaa4 is V.
18. The engineered AAV VP capsid polypeptide of embodiment 9, wherein Xaa5 is selected from T, W, and Y.
19. The engineered AAV VP capsid polypeptide of embodiment 9, wherein Xaa5 is Y.
20. The engineered AAV VP capsid polypeptide of embodiment 9, wherein Xaa6 is selected from L, N, R, and Y.
21. The engineered AAV VP capsid polypeptide of embodiment 9, wherein Xaa6 is Y.
22. The engineered AAV VP capsid polypeptide of embodiment 9, wherein Xaa7 is R.
23. The engineered AAV VP capsid polypeptide of embodiment 9, wherein Xaa8 is selected from S, T, and Y.
24. The engineered AAV VP capsid polypeptide of embodiment 9, wherein Xaa8 is T.
25. The engineered AAV VP capsid polypeptide of embodiment 9, wherein Xaa9 is selected from I, P, and R.
26. The engineered AAV VP capsid polypeptide of embodiment 9, wherein Xaa9 is I.
27. The engineered AAV VP capsid polypeptide of any one of embodiments 1-26, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 34991-SEQ ID NO: 35437.
28. The engineered AAV VP capsid polypeptide of embodiment 27, wherein the region from residue 581 to residue 589 of SEQ ID NO: 1 has a sequence of any one of SEQ ID NO: 34991-SEQ ID NO: 35437.
29. The engineered AAV VP capsid polypeptide of any one of embodiments 2-4, wherein Xaa1 has high volume.
30. The engineered AAV VP capsid polypeptide of embodiment 29, wherein Xaa1 is selected from F, W, and Y.
31. The engineered AAV VP capsid polypeptide of any one of embodiments 2-4, wherein Xaa1 has low mutability.
32. The engineered AAV VP capsid polypeptide of embodiment 31, wherein Xaa1 is selected from Y, F, L, and C.
33. The engineered AAV VP capsid polypeptide of any one of embodiments 2-4, wherein Xaa1 has high solubility.
34. The engineered AAV VP capsid polypeptide of embodiment 33, wherein Xaa1 is selected from W, F, I, and L.
35. The engineered AAV VP capsid polypeptide of any one of embodiments 2-4, wherein Xaa1 has low average flexibility.
36. The engineered AAV VP capsid polypeptide of embodiment 35, wherein Xaa1 is selected from F, M, and W.
37. The engineered AAV VP capsid polypeptide of any one of embodiments 2-4, wherein Xaa2 has low hydropathy.
38. The engineered AAV VP capsid polypeptide of embodiment 37, wherein Xaa2 is selected from P and Y.
39. The engineered AAV VP capsid polypeptide of any one of embodiments 2-4, wherein Xaa3 has low hydrophilicity.
40. The engineered AAV VP capsid polypeptide of embodiment 39, wherein Xaa3 is selected from Y, W, V, M, F, I, and L.
41. The engineered AAV VP capsid polypeptide of any one of embodiments 2-4, wherein Xaa3 has high solubility.
42. The engineered AAV VP capsid polypeptide of embodiment 41, wherein Xaa3 is selected from W, F, I, and L.
43. The engineered AAV VP capsid polypeptide of any one of embodiments 2-4, wherein Xaa6 has high volume.
44. The engineered AAV VP capsid polypeptide of embodiment 43, wherein Xaa6 is selected from W, R, K, M, I, and L.
45. The engineered AAV VP capsid polypeptide of any one of embodiments 2-4, wherein Xaa6 has high mol mass.
46. The engineered AAV VP capsid polypeptide of embodiment 45, wherein Xaa6 is selected from W.
47. The engineered AAV VP capsid polypeptide of any one of embodiments 2-4, wherein Xaa8 has high mol mass.
48. The engineered AAV VP capsid polypeptide of embodiment 47, wherein Xaa8 is selected from W, E, K, M, H, and Q.
49. The engineered AAV VP capsid polypeptide of any one of embodiments 2-4, wherein Xaa8 has high volume.
50. The engineered AAV VP capsid polypeptide of embodiment 49, wherein Xaa8 is selected from W, K, M, I, and L.
51. The engineered AAV VP capsid polypeptide of any one of embodiments 2-4, wherein Xaa8 has high goldman engelman steitz.
52. The engineered AAV VP capsid polypeptide of embodiment 51, wherein Xaa8 is selected from V and L.
53. The engineered AAV VP capsid polypeptide of any one of embodiments 2-4, wherein Xaa9 has high hydropathy.
54. The engineered AAV VP capsid polypeptide of embodiment 53, wherein Xaa9 is selected from V and I.
55. The engineered AAV VP capsid polypeptide of any one of embodiments 2-4, wherein Xaa9 has high solubility.
56. The engineered AAV VP capsid polypeptide of embodiment 55, wherein Xaa9 is selected from W, F, I, and L.
57. The engineered AAV VP capsid polypeptide of any one of embodiments 1-56, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 35438-SEQ ID NO: 36437.
58. The engineered AAV VP capsid polypeptide of embodiment 57, wherein the region from the residue corresponding to residue 581 to the residue corresponding to residue 589 inclusive has a sequence of any one of SEQ ID NO: 35438-SEQ ID NO: 36437.
59. The engineered AAV VP capsid polypeptide of any one of embodiments 1-58, wherein tropism for spinal cord tissue is measured as a relative accumulation of the rAAV virion in a spinal cord tissue as compared to a non-spinal cord tissue, wherein the non-spinal cord tissue consists collectively of CNS, liver, skeletal muscle, heart, lung, spleen, lymph node, bone marrow, mammary gland, skin, adrenal gland, thyroid, colon, and sciatic nerve.
60. The engineered AAV VP capsid polypeptide of embodiment 59, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.
61. The engineered AAV VP capsid polypeptide of any one of embodiments 59-60, wherein the higher tissue tropism is a 1.0005-fold to about a 1000-fold increased accumulation in the spinal cord tissue as compared to a non-spinal cord tissue.
62. The engineered AAV VP capsid polypeptide of embodiment 61, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the spinal cord tissue as compared to a non-spinal cord tissue.

### Series Q Embodiments—Spleen Tropic Capsids

1. An engineered adeno-associated virus (AAV) viral protein (VP) capsid polypeptide having an amino acid sequence at least 70% identical to SEQ ID NO: 1,
wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
wherein the engineered AAV VP capsid polypeptide is capable of assembling into a recombinant AAV virion (rAAV),
wherein the at least one mutation confers higher tropism for a spleen tissue on the rAAV as compared to an rAAV virion having a wildtype AAV5 VP capsid polypeptide of SEQ ID NO: 1, and
wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid. 3. An engineered adeno-associated virus (AAV) viral protein (VP) capsid polypeptide having an amino acid sequence of SEQ ID NO: 2,
wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
wherein the engineered AAV VP capsid polypeptide is capable of assembling into a recombinant AAV virion (rAAV), and
wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a spleen tissue as compared to an rAAV virion having a wildtype AAV5 VP capsid polypeptide of SEQ ID NO: 1. 5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 37438-SEQ ID NO: 40437. 6. The engineered AAV VP capsid polypeptide of embodiment 5, wherein the region from the residue corresponding to residue 581 to the residue corresponding to residue 589, inclusive, has a sequence of any one of SEQ ID NO: 37438-SEQ ID NO: 40437.
7. The engineered AAV VP capsid polypeptide of any one of embodiments 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 37438-SEQ ID NO: 40437.
8. The engineered AAV VP capsid polypeptide of embodiment 7, wherein the Xaa1 to Xaa9 region of SEQ ID NO: 2 has a sequence of any one of SEQ ID NO: 37438-SEQ ID NO: 40437.
9. The engineered AAV VP capsid polypeptide of any one of embodiments 2-4, wherein: Xaa1 is selected from the group consisting of C, F, H, I, L, P, W, and Y; or Xaa2 is selected from the group consisting of D, E, L, N, P, R, and W; or Xaa3 is selected from the group consisting of C, D, E, P, and W; or Xaa4 is selected from the group consisting of C, F, G, H, R, W and Y; or Xaa5 is selected from the group consisting of A, D, E, G, P, R, and W; or Xaa6 is selected from the group consisting of A, C, D, E, K, R, and W; or Xaa7 is selected from the group consisting of F, L, P, R, W, and Y; or Xaa8 is selected from the group consisting of E, I, K, L, P, R, and T; or Xaa9 is selected from the group consisting of C, H, M, T, V, and W; or any combination thereof.
10. The engineered AAV VP capsid polypeptide of embodiment 9, wherein Xaa1 is selected from C, F, P, W, and Y.
11. The engineered AAV VP capsid polypeptide of embodiment 9, wherein Xaa1 is selected from P, W, and Y.
12. The engineered AAV VP capsid polypeptide of embodiment 9, wherein Xaa1 is P.
13. The engineered AAV VP capsid polypeptide of embodiment 9, wherein Xaa2 is selected from D, E, and W.
14. The engineered AAV VP capsid polypeptide of embodiment 9, wherein Xaa2 is D.
15. The engineered AAV VP capsid polypeptide of embodiment 9, wherein Xaa3 is selected from D, P, and W.
16. The engineered AAV VP capsid polypeptide of embodiment 9, wherein Xaa3 is P.
17. The engineered AAV VP capsid polypeptide of embodiment 9, wherein Xaa4 is selected from C, H, and W.
18. The engineered AAV VP capsid polypeptide of embodiment 9, wherein Xaa4 is C.
19. The engineered AAV VP capsid polypeptide of embodiment 9, wherein Xaa5 is selected from D, E, G, and P.
20. The engineered AAV VP capsid polypeptide of embodiment 9, wherein Xaa5 is D.
21. The engineered AAV VP capsid polypeptide of embodiment 9, wherein Xaa6 is selected from C, K, and R.
22. The engineered AAV VP capsid polypeptide of embodiment 9, wherein Xaa6 is K.
23. The engineered AAV VP capsid polypeptide of embodiment 9, wherein Xaa7 is selected from L, P, and W.
24. The engineered AAV VP capsid polypeptide of embodiment 9, wherein Xaa7 is P.
25. The engineered AAV VP capsid polypeptide of embodiment 9, wherein Xaa8 is selected from P, R, and K.
26. The engineered AAV VP capsid polypeptide of embodiment 9, wherein Xaa8 is K.
27. The engineered AAV VP capsid polypeptide of embodiment 9, wherein Xaa9 is selected from C, T, and V.
28. The engineered AAV VP capsid polypeptide of embodiment 9, wherein Xaa9 is V.
29. The engineered AAV VP capsid polypeptide of any one of embodiments 1-28, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 37438-SEQ ID NO: 38437.
30. The engineered AAV VP capsid polypeptide of embodiment 29, wherein the region from residue 581 to residue 589 of SEQ ID NO: 1 has a sequence of any one of SEQ ID NO: 37438-SEQ ID NO: 38437.
31. The engineered AAV VP capsid polypeptide of any one of embodiments 2-4, wherein Xaa1 has low solubility.
32. The engineered AAV VP capsid polypeptide of embodiment 31, wherein Xaa1 is selected from D and P.
33. The engineered AAV VP capsid polypeptide of any one of embodiments 2-4, wherein Xaa1 has high solubility.
34. The engineered AAV VP capsid polypeptide of embodiment 33, wherein Xaa1 is selected from F, I, and L.
35. The engineered AAV VP capsid polypeptide of any one of embodiments 2-4, wherein Xaa1 has low hydropathy.
36. The engineered AAV VP capsid polypeptide of embodiment 35, wherein Xaa1 is selected from Y and P.
37. The engineered AAV VP capsid polypeptide of any one of embodiments 2-4, wherein Xaa1 has low mutability.
38. The engineered AAV VP capsid polypeptide of embodiment 37, wherein Xaa1 is selected from C, K, and P.
39. The engineered AAV VP capsid polypeptide of any one of embodiments 2-4, wherein Xaa2 has low solubility.
40. The engineered AAV VP capsid polypeptide of embodiment 39, wherein Xaa2 is selected from D, Q, and R.
41. The engineered AAV VP capsid polypeptide of any one of embodiments 2-4, wherein Xaa2 has low hydropathy.
42. The engineered AAV VP capsid polypeptide of embodiment 41, wherein Xaa2 is selected from D, E, R, K, H, N, and Q.
43. The engineered AAV VP capsid polypeptide of any one of embodiments 2-4, wherein Xaa2 has low charge.
44. The engineered AAV VP capsid polypeptide of embodiment 43, wherein Xaa2 is selected from D and E.
45. The engineered AAV VP capsid polypeptide of any one of embodiments 2-4, wherein Xaa2 has low volume.
46. The engineered AAV VP capsid polypeptide of embodiment 45, wherein Xaa2 is selected from T, N, P, and D.
47. The engineered AAV VP capsid polypeptide of any one of embodiments 2-4, wherein Xaa2 has high average flexibility.
48. The engineered AAV VP capsid polypeptide of embodiment 47, wherein Xaa2 is selected from D, E, R, P, G, Q, and S.
49. The engineered AAV VP capsid polypeptide of any one of embodiments 2-4, wherein Xaa3 has low solubility.
50. The engineered AAV VP capsid polypeptide of embodiment 49, wherein Xaa3 is selected from D, E, P, and N.
51. The engineered AAV VP capsid polypeptide of any one of embodiments 2-4, wherein Xaa3 has low hydropathy.
52. The engineered AAV VP capsid polypeptide of embodiment 51, wherein Xaa3 is selected from D, E, H, N, Q, and P.
53. The engineered AAV VP capsid polypeptide of any one of embodiments 2-4, wherein Xaa4 has low hydropathy.
54. The engineered AAV VP capsid polypeptide of embodiment 53, wherein Xaa4 is selected from K and R.
55. The engineered AAV VP capsid polypeptide of any one of embodiments 2-4, wherein Xaa5 has low solubility.
56. The engineered AAV VP capsid polypeptide of embodiment 55, wherein Xaa5 is selected from D, E, P, and N.
57. The engineered AAV VP capsid polypeptide of any one of embodiments 2-4, wherein Xaa5 has high average flexibility.
58. The engineered AAV VP capsid polypeptide of embodiment 57, wherein Xaa5 is selected from D, E, R, P, G, Q, and S.
59. The engineered AAV VP capsid polypeptide of any one of embodiments 2-4, wherein Xaa6 has low mutability.
60. The engineered AAV VP capsid polypeptide of embodiment 59, wherein Xaa6 is selected from C.
61. The engineered AAV VP capsid polypeptide of any one of embodiments 2-4, wherein Xaa8 has high surface accessibility.
62. The engineered AAV VP capsid polypeptide of embodiment 61, wherein Xaa8 is selected from E, R, and K.
63. The engineered AAV VP capsid polypeptide of any one of embodiments 2-4, wherein Xaa8 has low solubility.
64. The engineered AAV VP capsid polypeptide of embodiment 63, wherein Xaa8 is selected from E, P, R, K, N, Q.
65. The engineered AAV VP capsid polypeptide of any one of embodiments 2-4, wherein Xaa8 has medium volume.
66. The engineered AAV VP capsid polypeptide of embodiment 65, wherein Xaa8 is selected from E, D, R, K, V, P, M, I, L, H, N, Q, and T.
67. The engineered AAV VP capsid polypeptide of any one of embodiments 2-4, wherein Xaa9 has medium mol mass.
68. The engineered AAV VP capsid polypeptide of embodiment 67, wherein Xaa9 is selected from E, D, K, M, I, L, H, N.
69. The engineered AAV VP capsid polypeptide of any one of embodiments 1-68, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 38438-SEQ ID NO: 39437.
70. The engineered AAV VP capsid polypeptide of embodiment 69, wherein the region from the residue corresponding to residue 581 to the residue corresponding to residue 589 inclusive has a sequence of any one of SEQ ID NO: 38438-SEQ ID NO: 39437.
71. The engineered AAV VP capsid polypeptide of any one of embodiments 1-70, wherein tropism for spleen tissue is measured as a relative accumulation of the rAAV virion in a spleen tissue as compared to a non-spleen tissue, wherein the non-spleen tissue consists collectively of CNS, liver, skeletal muscle, heart, lung, lymph node, bone marrow, mammary gland, skin, adrenal gland, thyroid, colon, sciatic nerve, and spinal cord.
72. The engineered AAV VP capsid polypeptide of embodiment 71, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.
73. The engineered AAV VP capsid polypeptide of any one of embodiments 71-72, wherein the higher tissue tropism is a 1.0005-fold to about a 1000-fold increased accumulation in the spleen tissue as compared to a non-spleen tissue.
74. The engineered AAV VP capsid polypeptide of embodiment 73, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the spleen tissue as compared to a non-spleen tissue.

### Series R Embodiments—Thyroid Gland Tropic Capsids

1. An engineered adeno-associated virus (AAV) viral protein (VP) capsid polypeptide having an amino acid sequence at least 70% identical to SEQ ID NO: 1,
wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
wherein the engineered AAV VP capsid polypeptide is capable of assembling into a recombinant AAV virion (rAAV),
wherein the at least one mutation confers higher tropism for a thyroid gland tissue on the rAAV as compared to an rAAV virion having a wildtype AAV5 VP capsid polypeptide of SEQ ID NO: 1, and
wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8. 2. The engineered AAV VP capsid polypeptide of embodiment 1, wherein the engineered AAV VP capsid polypeptide has a sequence of SEQ ID NO: 2 and wherein Xaa1, Xaa2, Xaa3, Xaa4, Xaa5, Xaa6, Xaa7, Xaa8, and Xaa9 are each independently selected from any amino acid. 3. An engineered adeno-associated virus (AAV) viral protein (VP) capsid polypeptide having an amino acid sequence of SEQ ID NO: 2,
wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V,
wherein the engineered AAV VP capsid polypeptide is capable of assembling into a recombinant AAV virion (rAAV), and
wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8. 4. The engineered AAV VP capsid polypeptide of embodiment 3, wherein the rAAV has higher tropism for a thyroid gland tissue as compared to an rAAV virion having a wildtype AAV5 VP capsid polypeptide of SEQ ID NO: 1. 5. The engineered AAV VP capsid polypeptide of embodiment 1, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 40438-SEQ ID NO: 43437. 6. The engineered AAV VP capsid polypeptide of embodiment 5, wherein the region from the residue corresponding to residue 581 to the residue corresponding to residue 589, inclusive, has a sequence of any one of SEQ ID NO: 40438-SEQ ID NO: 43437. 7. The engineered AAV VP capsid polypeptide of embodiment 2-4, wherein the Xaa1 to Xaa9 region of SEQ ID NO: 2 has a sequence that is at least 70%, at least 75%, at least 80%, at least 85%, at least 90%, at least 92%, at least 95%, at least 97%, or at least 99% identical to any one of SEQ ID NO: 40438-SEQ ID NO: 43437. 8. The engineered AAV VP capsid polypeptide of embodiment 7, wherein the Xaa1 to Xaa9 region of SEQ ID NO: 2 has a sequence of any one of SEQ ID NO: 40438-SEQ ID NO: 43437.
9. The engineered AAV VP capsid polypeptide of any one of embodiments 2-4, wherein:
   Xaa1 is selected from the group consisting of A, K, M, N, Q, or R; or
   Xaa2 is selected from the group consisting of A, F, K, L, M, T, V, or W; or
   Xaa3 is selected from the group consisting of A, I, K, R, S, T, V, or W; or
   Xaa4 is selected from the group consisting of A, D, E, I, P, or V; or
   Xaa5 is selected from the group consisting of F, I, M, Q, V, or Y; or
   Xaa6 is selected from the group consisting of H, M, N, or Y; or
   Xaa7 is selected from the group consisting of H, I, N, Q, S, or W; or
   Xaa8 is selected from the group consisting of A, D, F, Q, S, or Y; or
   Xaa9 is selected from the group consisting of A, Q, S, or Y; or
   any combination thereof.
10. The engineered AAV VP capsid polypeptide of embodiment 9, wherein Xaa1 is selected from K, N and Q.
11. The engineered AAV VP capsid polypeptide of embodiment 9, wherein Xaa1 is K.
12. The engineered AAV VP capsid polypeptide of embodiment 9, wherein Xaa2 is selected from F, V, and W.
13. The engineered AAV VP capsid polypeptide of embodiment 9, wherein Xaa2 is W.
14. The engineered AAV VP capsid polypeptide of embodiment 9, wherein Xaa3 is selected from A, R and T.
15. The engineered AAV VP capsid polypeptide of embodiment 9, wherein Xaa3 is R.
16. The engineered AAV VP capsid polypeptide of embodiment 9, wherein Xaa4 is selected from A, E, and I.
17. The engineered AAV VP capsid polypeptide of embodiment 9, wherein Xaa4 is A.
18. The engineered AAV VP capsid polypeptide of embodiment 9, wherein Xaa5 is M, V, or Y.
19. The engineered AAV VP capsid polypeptide of embodiment 9, wherein Xaa5 is M.
20. The engineered AAV VP capsid polypeptide of embodiment 9, wherein Xaa6 is N.
21. The engineered AAV VP capsid polypeptide of embodiment 9, wherein Xaa7 is selected from H, I, and N.
22. The engineered AAV VP capsid polypeptide of embodiment 9, wherein Xaa7 is H.
23. The engineered AAV VP capsid polypeptide of embodiment 9, wherein Xaa8 is selected from A, F, and S.
24. The engineered AAV VP capsid polypeptide of embodiment 9, wherein Xaa8 is F.
25. The engineered AAV VP capsid polypeptide of embodiment 9, wherein Xaa9 is selected from A and S.
26. The engineered AAV VP capsid polypeptide of embodiment 9, wherein Xaa9 is A.
27. The engineered AAV VP capsid polypeptide of any one of embodiments 1-26, wherein the region from the residue corresponding to residue 581 to the residue corresponding to residue 589, inclusive, has a sequence that is at least 70%, at least 75%, at least 80%, at least 85%, at least 90%, at least 92%, at least 95%, at least 97%, or at least 99% identical to any one of SEQ ID NO: 40438-SEQ ID NO: 41437.
28. The engineered AAV VP capsid polypeptide of embodiment 27, wherein the region from residue 581 to residue 589 of SEQ ID NO: 1 has a sequence of any one of SEQ ID NO: 40438-SEQ ID NO: 41437.
29. The engineered AAV VP capsid polypeptide of any one of embodiments 2-4, wherein Xaa1 has high mutability.
30. The engineered AAV VP capsid polypeptide of embodiment 29, wherein Xaa1 is N.
31. The engineered AAV VP capsid polypeptide of any one of embodiments 2-4, wherein Xaa2 has low surface accessibility.
32. The engineered AAV VP capsid polypeptide of embodiment 31, wherein Xaa2 is selected from F, G, and M.
33. The engineered AAV VP capsid polypeptide of any one of embodiments 2-4, wherein Xaa3 has high solubility.
34. The engineered AAV VP capsid polypeptide of embodiment 33, wherein Xaa3 is selected from F.
35. The engineered AAV VP capsid polypeptide of any one of embodiments 2-4, wherein Xaa3 has low mutability.
36. The engineered AAV VP capsid polypeptide of embodiment 35, wherein Xaa3 is selected from Y, F, L, and C.
37. The engineered AAV VP capsid polypeptide of any one of embodiments 2-4, wherein Xaa3 has medium mol mass.
38. The engineered AAV VP capsid polypeptide of embodiment 37, wherein Xaa3 is selected from D, E, R, K, V, P, M, I, L, N, Q, T, and C.
39. The engineered AAV VP capsid polypeptide of any one of embodiments 2-4, wherein Xaa3 has low surface accessibility.
40. The engineered AAV VP capsid polypeptide of embodiment 39, wherein Xaa3 is selected from V, I, L, and C.
41. The engineered AAV VP capsid polypeptide of any one of embodiments 2-4, wherein Xaa4 has high goldman engelman steitz.
42. The engineered AAV VP capsid polypeptide of embodiment 41, wherein Xaa4 is selected from L and V.
43. The engineered AAV VP capsid polypeptide of any one of embodiments 2-4, wherein Xaa4 has low surface accessibility.
44. The engineered AAV VP capsid polypeptide of embodiment 43, wherein Xaa4 is selected from V, M, A, G, F, I, and L.
45. The engineered AAV VP capsid polypeptide of any one of embodiments 2-4, wherein Xaa4 has low mol mass.
46. The engineered AAV VP capsid polypeptide of embodiment 45, wherein Xaa4 is selected from D, A, G, I, L, and N.
47. The engineered AAV VP capsid polypeptide of any one of embodiments 2-4, wherein Xaa5 has high solubility.
48. The engineered AAV VP capsid polypeptide of embodiment 47, wherein Xaa5 is selected from C, L, F, M, V, and Y.
49. The engineered AAV VP capsid polypeptide of any one of embodiments 2-4, wherein Xaa5 has low solubility.
50. The engineered AAV VP capsid polypeptide of embodiment 49, wherein Xaa5 is selected from D.
51. The engineered AAV VP capsid polypeptide of any one of embodiments 2-4, wherein Xaa5 has low average flexibility.
52. The engineered AAV VP capsid polypeptide of embodiment 51, wherein Xaa5 is selected from F, M, and W.
53. The engineered AAV VP capsid polypeptide of any one of embodiments 2-4, wherein Xaa6 has low average flexibility.
54. The engineered AAV VP capsid polypeptide of embodiment 53, wherein Xaa6 is selected from F, M, and W.
55. The engineered AAV VP capsid polypeptide of any one of embodiments 2-4, wherein Xaa7 has high mutability.
56. The engineered AAV VP capsid polypeptide of embodiment 55, wherein Xaa7 is selected from N.
57. The engineered AAV VP capsid polypeptide of any one of embodiments 2-4, wherein Xaa7 has low volume.
58. The engineered AAV VP capsid polypeptide of embodiment 57, wherein Xaa7 is selected from P, N, and T.
59. The engineered AAV VP capsid polypeptide of any one of embodiments 2-4, wherein Xaa8 has low average flexibility.
60. The engineered AAV VP capsid polypeptide of embodiment 59, wherein Xaa8 is selected from F, M, and W.
61. The engineered AAV VP capsid polypeptide of any one of embodiments 2-4, wherein Xaa8 has low surface accessibility.
62. The engineered AAV VP capsid polypeptide of embodiment 61, wherein Xaa8 is selected from M, G, and F.
63. The engineered AAV VP capsid polypeptide of any one of embodiments 2-4, wherein Xaa9 has low mutability.
64. The engineered AAV VP capsid polypeptide of embodiment 63, wherein Xaa9 is selected from R, K, P, H, and C.
65. The engineered AAV VP capsid polypeptide of any one of embodiments 2-4, wherein Xaa9 has low hydropathy.
66. The engineered AAV VP capsid polypeptide of embodiment 65, wherein Xaa9 is selected from R.
67. The engineered AAV VP capsid polypeptide of any one of embodiments 1-66, wherein the region from the residue corresponding to residue 581 to the residue corresponding to residue 589 inclusive has a sequence having at least 70%, at least 75%, at least 80%, at least 85%, at least 90%, at least 92%, at least 95%, at least 97%, or at least 99% identical to any one of SEQ ID NO: 41438-SEQ ID NO: 42437.
68. The engineered AAV VP capsid polypeptide of embodiment 67, wherein the region from the residue corresponding to residue 581 to the residue corresponding to residue 589 inclusive has a sequence of any one of SEQ ID NO: 41438-SEQ ID NO: 42437.
69. The engineered AAV VP capsid polypeptide of any one of embodiments 1-68, wherein tropism for thyroid gland tissue is measured as a relative accumulation of the rAAV virion in a thyroid gland tissue as compared to a non-thyroid gland tissue, wherein the non-thyroid gland tissue consists collectively of CNS, liver, skeletal muscle, heart, lung, spleen, lymph node, bone marrow, mammary gland, skin, adrenal gland, colon, sciatic nerve, and spinal cord.
70. The engineered AAV VP capsid polypeptide of embodiment 69, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.
71. The engineered AAV VP capsid polypeptide of any one of embodiments 69-70, wherein the higher tissue tropism is a 1.0005-fold to about a 1000-fold increased accumulation in the thyroid gland tissue as compared to a non-thyroid gland tissue.
72. The engineered AAV VP capsid polypeptide of embodiment 71, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the thyroid gland tissue as compared to a non-thyroid gland tissue.

### Series S Embodiments

1. An engineered adeno-associated virus (AAV) VP capsid polypeptide having the amino acid sequence of SEQ ID NO:2,
   wherein amino acid residues Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V;
   wherein the capsid polypeptide is capable of assembling into a recombinant AAV virion (rAAV); and
   wherein the polypeptide does not have the sequence of any of SEQ ID NO:1, SEQ ID NO: 3, SEQ ID NO:4, SEQ ID NO:5, SEQ ID NO:6, SEQ ID NO:7, and SEQ ID NO:8.
2. An engineered adeno-associated virus (AAV) VP capsid polypeptide having at least one mutation in a residue corresponding to residue 581 to residue 589 in SEQ ID NO: 1, wherein the capsid polypeptide is capable of assembling into a recombinant AAV virion (rAAV) and wherein the polypeptide mutation confers tissue tropism on the recombinant rAAV for a first tissue as compared to a second tissue and wherein the AAV VP capsid polypeptide does not have the sequence of any of SEQ ID NO:3, SEQ ID NO:4, SEQ ID NO:5, SEQ ID NO:6, SEQ ID NO:7, and SEQ ID NO:8.
3. An engineered AAV VP capsid polypeptide comprising a polypeptide sequence represented by the formula: (A)-(X)-(B)
   wherein:
   (A) is the polypeptide sequence of SEQ ID NO:47438 (VAYNVGGQMATNNQSSTTAP residues 561-580 of SEQ ID NO:2);
   (X) is the polypeptide sequence comprising amino acid residues Xaa1, Xaa2, Xaa3, Xaa4, Xaa5, Xaa6, Xaa7, Xaa8, and Xaa9 of SEQ ID NO:2; and
   (B) is the polypeptide sequence of SEQ ID NO:47439 (IVPGSVWMERDVYLQGPIWA residues 590-609 of SEQ ID NO:2;
   wherein Xaa1, Xaa2, Xaa3, Xaa4, Xaa5, Xaa6, Xaa7, Xaa8, and Xaa9 are each independently selected from A, R, N, D, C, E, Q, G, H, I, L, K, M, F, P, S, T, W, Y, and V; and
   wherein the capsid polypeptide is capable of assembling into a recombinant AAV virion (rAAV); and;
   wherein the polypeptide does not have the sequence of any of SEQ ID NO:1, SEQ ID NO: 3, SEQ ID NO:4, SEQ ID NO:5, SEQ ID NO:6, SEQ ID NO:7, and SEQ ID NO:8.
4. The engineered AAV VP capsid polypeptide of embodiment 3, comprising a polypeptide sequence represented by the formula: (A)-(X)-(B) wherein:
   (A) is the polypeptide sequence of SEQ ID NO: 47438 (residues 561-580 of SEQ ID NO: 2 VAYNVGGQMATNNQSSTTAP);
   (X) is a polypeptide sequence selected from the list of polypeptides in Table 8 (SEQ ID NOs: 115-1114)′ or Table 10 (SEQ ID NOs: 7118-8117) that confers CNS tissue tropism on a recombinant AAV virion (rAAV); and
   (B) is the polypeptide sequence of SEQ ID NO: 47439 (residues 590-609 of SEQ ID NO: 2: (IVPGSVWMERDVYLQGPIWA)); and
   wherein the capsid polypeptide is capable of assembling into the rAAV and,
the capsid does not have the sequence of any of SEQ ID NO:1, SEQ ID NO:3, SEQ ID NO:4, SEQ ID NO:5, SEQ ID NO:6, SEQ ID NO:7, and SEQ ID NO:8. 5. The engineered AAV VP capsid polypeptide of embodiment 4, wherein the CNS tissue is selected from the group consisting of hippocampus: (dentate gyrus, CA1 and CA3); cerebellum, hypothalamus, cortex: (occipital, temporal and forebrain); substantia nigra, thalamus, and any combination thereof.
6. The recombinant AAV VP capsid polypeptide of embodiment 3, comprising a polypeptide sequence represented by the formula: (A)-(X)-(B) wherein:
   (A) is the polypeptide sequence of SEQ ID NO: 47438 (residues 561-580 of SEQ ID NO: 2: (VAYNVGGQMATNNQSSTTAP));
   (X) is a polypeptide sequence selected from any of the polypeptides of SEQ ID NO:115-1114 or 1118-47437 that confer corresponding tissue tropism on a recombinant AAV virion (rAAV); and
   (B) is the polypeptide sequence of SEQ ID NO: 47439 (residues 590-609 of SEQ ID NO: 2: (IVPGSVWMERDVYLQGPIWA)); and
   wherein the capsid polypeptide is capable of assembling into the rAAV and, the capsid does not have the sequence of any of SEQ ID NO:1, SEQ ID NO:3, SEQ ID NO:4, SEQ ID NO:5, SEQ ID NO:6, SEQ ID NO:7, and SEQ ID NO:8. 7. The engineered AAV VP capsid polypeptide of any of the previous embodiments, wherein the AAV VP capsid polypeptide is an AAV5 VP1 capsid polypeptide. 8. The engineered AAV VP capsid polypeptide of any of the previous embodiments, wherein the capsid confers upon a recombinant AAV virion (rAAV), increased tissue targeting for any tissue selected from the following tissues:
   adipose, adrenal gland, aorta, brain (including hippocampus: dentate gyrus, CA1 and CA3; cerebellum, caudate, putamen, midbrain, pons, hypothalamus, cortex-including occipital, temporal and forebrain; substantia nigra, thalamus, and any combinations thereof), bone marrow, cecum, colon, dorsal root ganglion, duodenum, epididymis, esophagus, eye, gallbladder, heart, ileum, jejunum, kidney, lung, lymph nodes, mammary gland, ovary, pancreas, parathyroid gland, peripheral nerve, pituitary, prostate, salivary gland, seminal vesicle, skeletal muscle, skin, spinal cord, spleen, stomach, testis, thymus, thyroid, trachea, urinary bladder, uterus, and vagina.
compared to a control virion comprising a capsid with any of the following sequences: SEQ ID NO: 1, SEQ ID NO:3, SEQ ID NO:4, SEQ ID NO:5, SEQ ID NO:6, SEQ ID NO:7, and SEQ ID NO: 8. 9. The engineered AAV VP capsid polypeptide of embodiment 2, wherein the rAAV exhibits from about a 1.0005-fold to about a 1000-fold increased accumulation in the first tissue as compared to the second tissue. 10. The engineered AAV VP capsid polypeptide of embodiment 9, wherein the rAAV exhibits at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the first tissue as compared to the second tissue. 11. The engineered AAV VP capsid polypeptide of any of embodiments 1 or 3-8, wherein the rAAV exhibits from about a 1.0005-fold to about a 1000-fold increased accumulation in the tissue. 12. The engineered AAV VP capsid polypeptide of embodiment 11, wherein the rAAV exhibits at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the tissue. 13. The engineered AAV VP capsid polypeptide of any of embodiments 1-3 or 6-12, wherein at least one mutant residue is:
   Xaa1 and Xaa1 is selected from A, G, K, M, N, Q, R, S, or T;
   Xaa2 and Xaa2 is selected from A, C, H, I, K, S, T, or V;
   Xaa3 and Xaa3 is selected from A, G, H, K, M, N, Q, R, S, T, or V;
   Xaa4 and Xaa4 is selected from L, M, P, Q, R, T, or W;
   Xaa5 and Xaa5 is selected from F, H, I, K, M, T, or Y;
   Xaa6 and Xaa6 is selected from E, G, H, L, M, N, Q, T, or W;
   Xaa7 and Xaa7 is selected from A, C, G, H, L, M, R or S;
   Xaa8 and Xaa8 is selected from A, C, D, F, G, H, M, Q, S, V, W, or Y;
   Xaa9 and Xaa9 is selected from A, C, E, G, H, M, N, P, Q, S, V, or W;
   or any combination thereof,
   wherein the capsid polypeptide is capable of assembling into a recombinant AAV virion (rAAV) and wherein the rAAV is capable of exhibiting tissue tropism for liver tissue.
14. The engineered AAV VP capsid polypeptide of any of embodiments 1-12, wherein:
   Xaa1 excludes A, G, K, M, N, Q, R, S, or T;
   Xaa2 excludes A, C, H, I, K, S, T, or V;
   Xaa3 excludes A, G, H, K, M, N, Q, R, S, T, or V;
   Xaa4 excludes L, M, P, Q, R, T, or W;
   Xaa5 excludes F, H, I, K, M, T, or Y;
   Xaa6 excludes E, G, H, L, M, N, Q, T, or W;
   Xaa7 excludes A, C, G, H, L, M, R or S.
   Xaa8 excludes A, C, D, F, G, H, M, Q, S, V, W, or Y;
   Xaa9 excludes A, C, E, G, H, M, N, P, Q, S, V, or W.
   or any combination thereof,
wherein the capsid polypeptide is capable of assembling into a recombinant AAV virion (rAAV) and exhibits less targeting or tropism to liver tissue compared to a rAAV having a VP1 capsid polypeptide having the sequence of SEQ ID NO:1. 15. The engineered AAV VP capsid polypeptide of any of the previous embodiments, having at least one residue corresponding to residue 581 to residue 589 in SEQ ID NO: 2, wherein:
   Xaa1 is A, D, E, G, L, M, N, Q, S, T, or V;
   Xaa2 is A, C, D, E, G, H, I, N, P, Q, S, T, or V;
   Xaa3 is A, D, E, G, H, M, N, Q, S, T, or V;
   Xaa4 is A, D, E, G, H, N, P, Q, S, or T;
   Xaa5 is A, C, D, E, G, H, N, Q, S, T, or Y;
   Xaa6 is A, D, E, G, H, N, P, Q, S, or T;
   Xaa7 is s A, C, D, E, G, H, N, Q, S, or T;
   Xaa8 is A, C, D, E, G, H, N, Q, S, or T;
   Xaa9 is A, D, E, G, H, N, P, Q, S, or T;
   or any combination thereof,
wherein the capsid polypeptide is capable of assembling into a recombinant AAV virion (rAAV).
16. The recombinant capsid polypeptide of any preceding embodiment, further comprising one or more mutations or substitutions at an amino acid residue outside of the 581-589 region, wherein the one or more mutations or substitutions at an amino acid residue outside of the 581-589 region confers improved manufacturability, improved viral assembly, improved tissue targeting/tropism, or any combination thereof. 17. A polynucleotide encoding any of the recombinant adeno-associated virus (AAV) VP capsid polypeptides of embodiments 1-16. 18. A recombinant AAV virion (rAAV), the virion comprising an AAV VP capsid polypeptide of any of embodiments 1-15. 19. The rAAV virion of embodiment 18, wherein the rAAV has reduced tropism for human liver as compared to a rAAV having a VP1 capsid polypeptide having the sequence of SEQ ID NO: 1. 20. The rAAV virion of embodiment 18 or embodiment 19, wherein the rAAV has increased ability to cross the blood-brain barrier following intravenous administration as compared to a rAAV having a VP1 capsid polypeptide having the sequence of SEQ ID NO:1. 21. The rAAV virion of any one of embodiments 18-20 wherein the rAAV has increased ability to infect one or more brain regions selected from hippocampus, dentate gyrus, cerebral cortex, temporal cortex, occipital cortex, thalamus, forebrain, substantia nigra, hypothalamus, and cerebellum, following intravenous, intrathecal, intracerebral ventricular, or intracisternal magna administration as compared to a rAAV having a VP1 capsid polypeptide having the sequence of SEQ ID NO:1.
22. The rAAV virion of any one of embodiments 18-21, wherein the rAAV has increased ability to infect human retinal cells following intravitreal injection as compared to a rAAV having a VP1 capsid polypeptide having the sequence of SEQ ID NO:1.
23. The rAAV virion of any one of embodiments 18, wherein the rAAV has increased ability to infect human skeletal muscle following intravenous administration as compared to a rAAV having a VP1 capsid polypeptide having the sequence of SEQ ID NO:1.
24. The rAAV virion of any one of embodiments 18, wherein the rAAV has increased tropism for human liver as compared to a rAAV having a VP1 capsid polypeptide having the sequence of SEQ ID NO:1. 25. The rAAV virion of any one of embodiments 18-24, wherein the virion further comprises a vector genome, the vector genome comprising a therapeutic polynucleotide encoding any of the following: a therapeutic RNA selected from a guide RNA or a tRNA, or transgene encoding a protein under control of regulatory sequences that direct transgene expression in infected human cells. 26. The rAAV virion of embodiment 25, wherein the transgene encodes a protein selected from the transgene products of Table 1. 27. A composition comprising an AAV virion comprising the engineered capsid polypeptide of any one of embodiments 1-16, within which is packaged the therapeutic polynucleotide of embodiment 25. 28. A composition comprising an AAV virion comprising the engineered capsid polypeptide of any one of embodiments 1-16, within which is packaged a therapeutic polynucleotide encoding any of the following: a therapeutic RNA selected from a guide RNA or a tRNA, or transgene encoding a protein under control of regulatory sequences that direct transgene expression in infected human cells. 29. The rAAV virion of any one of embodiments 18-26, wherein the virion further comprises a vector genome, the vector genome comprising a therapeutic polynucleotide encoding any of the following: a therapeutic RNA selected from a guide RNA or a tRNA, or transgene encoding a protein under control of regulatory sequences that direct transgene expression in infected human cells. 30. The rAAV virion of embodiment 29, wherein the transgene encodes a protein selected from the transgene products of Table 1.
31. A pharmaceutical composition comprising the rAAV of embodiment 29 or embodiment 30 and a pharmaceutically acceptable carrier. 32. A method of treatment, comprising:
   administering an effective amount of the pharmaceutical composition of embodiment 31 to a patient in need thereof. 33. The method of embodiment 32, wherein the effective amount of the rAAV is less than the effective amount of a wild type rAAV. 34. The method of embodiment 32, wherein the effective amount of the rAAV is less than the effective amount of an otherwise comparable rAAV lacking one or more than one mutation or substitution at a position corresponding to residue 581 to residue 589 of SEQ ID NO: 1. 35. The method of embodiment 34, wherein the effective amount of the rAAV results in lower toxicity in the patient as compared to the effective amount of the wild type rAAV, the otherwise comparable rAAV, or both.
36. The method of embodiment 32, wherein the effective amount is at least from 1×10⁵ viral genomes/kg patient weight to 5×10¹⁴ viral genomes/kg.
37. The method of any one of embodiments 32-36, wherein the rAAV is administered intravenously.
38. The method of any one of embodiments 32-36, wherein the rAAV is administered intrathecally.
39. The method of any one of embodiments 32-36, wherein the rAAV is administered by intracisternal magna administration.
40. The method of any one of embodiments 32-36, wherein the rAAV is administered by intravitreal injection.
41. The rAAV virion according to any one of embodiments 18-26 or 29-30, for use in treating a disease in a patient.
42. The rAAV virion for use according to embodiment 41, for use in treating any of the diseases in Table 1.
43. The rAAV virion for use according to embodiment 41, for use in treating a CNS disease.
44. The rAAV virion for use according to embodiment 43, wherein the CNS disease is selected from the conditions listed in Table 1.
45. The rAAV virion for use according to embodiment 43 or 44, wherein the engineered AAV VP capsid polypeptide comprises any one of the mutations recited in Table 8 (SEQ ID NO:115-1114), Table 10 (SEQ ID NO: 7118-8117), Table 39 (SEQ ID NO: 8118-9117) or Table 74 (SEQ ID NO: 9118-10117).
46. The rAAV virion for use according to embodiment 45, wherein the engineered VP1 capsid polypeptide does not have the sequence of any of SEQ ID NO:1, SEQ ID NO:3, SEQ ID NO:4, SEQ ID NO:5, SEQ ID NO:6, SEQ ID NO:7, and SEQ ID NO:8.
47. The rAAV virion for use according to embodiment 42, wherein the virion comprises a therapeutic polynucleotide encoding a target from any of the targets listed in Table 1.

### Series T Embodiments—AAV5

1. The engineered AAV VP capsid polypeptide of any of the embodiments described herein, wherein the engineered AAV VP capsid polypeptide is an engineered AAV5 VP capsid polypeptide.
2. The engineered AAV VP capsid polypeptide of embodiment 1, wherein the mutation is a substitution.

### Series U Embodiments—Assembly

1. The engineered AAV VP capsid polypeptide of any of the embodiments described herein, wherein:
   Xaa1 is selected from the group consisting of A, D, E, G, L, M, N, Q, S, T, or V; or
      Xaa2 is selected from the group consisting of A, C, D, E, G, H, I, N, P, Q, S, T, or V; or
      Xaa3 is selected from the group consisting of A, D, E, G, H, M, N, Q, S, T, or V; or
      Xaa4 is selected from the group consisting of A, D, E, G, H, N, P, Q, S, or T; or
      Xaa5 is selected from the group consisting of A, C, D, E, G, H, N, Q, S, T, or Y; or
      Xaa6 is selected from the group consisting of A, D, E, G, H, N, P, Q, S, or T; or
      Xaa7 is selected from the group consisting of A, C, D, E, G, H, N, Q, S, or T; or
      Xaa8 is selected from the group consisting of A, C, D, E, G, H, N, Q, S, or T; or
      Xaa9 is selected from the group consisting of A, D, E, G, H, N, P, Q, S, or T; or any combination thereof.
2. The engineered AAV VP capsid polypeptide of embodiment 1, wherein Xaa1 is A, D, E, M, or T.
3. The engineered AAV VP capsid polypeptide of embodiment 1, wherein Xaa1 is E.
4. The engineered AAV VP capsid polypeptide of embodiment 1, wherein Xaa2 is A, S, T, or V.
5. The engineered AAV VP capsid polypeptide of embodiment 1, wherein Xaa2 is A.
6. The engineered AAV VP capsid polypeptide of embodiment 1, wherein Xaa3 is D, E, N, Q or T.
7. The engineered AAV VP capsid polypeptide of embodiment 1, wherein Xaa3 is D or T.
8. The engineered AAV VP capsid polypeptide of embodiment 1, wherein Xaa4 is D, E, P, or Q.
9. The engineered AAV VP capsid polypeptide of embodiment 1, wherein Xaa4 is E.
10. The engineered AAV VP capsid polypeptide of any one of claims X-Y, wherein Xaa5 is D, E, N, Q or T.
11. The engineered AAV VP capsid polypeptide of embodiment 1, wherein Xaa5 is N.
12. The engineered AAV VP capsid polypeptide of embodiment 1, wherein Xaa6 is D, N, or Q.
13. The engineered AAV VP capsid polypeptide of embodiment 1, wherein Xaa6 is D.
14. The engineered AAV VP capsid polypeptide of any one of claims X-Y, wherein Xaa7 is A, D, E or G.
15. The engineered AAV VP capsid polypeptide of embodiment 1, wherein Xaa7 is A.
16. The engineered AAV VP capsid polypeptide of any one of claims X-Y, wherein Xaa8 is A, D, G, or S.
17. The engineered AAV VP capsid polypeptide of embodiment 1, wherein Xaa8 is G.
18. The engineered AAV VP capsid polypeptide of embodiment 1, wherein Xaa9 is A, D, G, or P.
19. The engineered AAV VP capsid polypeptide of embodiment 1, wherein Xaa9 is G.

### Series V Embodiments—Recombinant AAV Virions

1. A recombinant AAV virion (rAAV), comprising:
   the engineered AAV VP capsid polypeptide of embodiments described herein assembled into a capsid; and
   a payload,
   wherein the engineered AAV VP capsid polypeptide encapsidates the payload.
2. The recombinant rAAV of embodiment 1, wherein the payload comprises a therapeutic polynucleotide, optionally wherein the therapeutic polynucleotide encodes a transgene or a genome modifying entity.
3. The rAAV of embodiment 2, wherein the therapeutic polynucleotide encodes a guide RNA, a tRNA, a suppressor tRNA, a siRNA, a miRNA, an mRNA, a shRNA, a circular RNA, or an antisense oligonucleotide (ASO), a ribozyme, a DNAzyme, an aptamer, or any combination thereof.
4. The rAAV of embodiment 3, wherein the therapeutic polynucleotide encodes a linear therapeutic polynucleotide or a circular therapeutic polynucleotide.
5. The rAAV of embodiment 2, wherein the transgene is selected from TABLE 1.
6. The rAAV of embodiment 2, wherein the genome modifying entity comprises a CRISPR/Cas system, an adenosine deaminase acting on RNA (ADAR) enzyme, a transcriptional activator, or a transcriptional repressor.
7. The rAAV of embodiment 6, wherein the CRISPR/Cas system comprises a Cas3, Cas8, Cas10, Cas9, Cas4, Cas12, or Cas13.
8. The rAAV of embodiment 6, wherein the ADAR is human ADAR1 or human ADAR2.
9. The rAAV of embodiment 6, wherein the transcriptional activator is VP64.
10. The rAAV of embodiment 6, wherein the transcriptional repressor is KRAB.
11. A pharmaceutical composition comprising the rAAV virion of any one of embodiments 1-10, and a pharmaceutically acceptable carrier.
12. A method of treatment, comprising administering a therapeutically effective amount of the pharmaceutical composition of embodiment 11, to a subject in need thereof.
13. The pharmaceutical composition of embodiment 12, for use in treating a subject in need thereof, wherein the use comprises administering a therapeutically effective amount of the pharmaceutical composition of embodiment 11, to the subject.
14. The rAAV virion of any one of the embodiments 1-10, for use in the manufacture of a medicament for treating a subject in need thereof by administration of a therapeutically effective amount thereof.
15. The method or use of any one of the embodiments 12-14, wherein the therapeutically effective amount of the rAAV virion is less than the therapeutically effective amount of a rAAV virion having a wildtype AAV5 VP capsid polypeptide of SEQ ID NO: 1 encapsidating the same payload, wherein the rAAV virion having the wildtype AAV5 VP capsid polypeptide of SEQ ID NO: 1 is administered by the same route of administration.
16. The method or use of any one of the embodiments 12-15, wherein the therapeutically effective amount of the rAAV virion results in lower toxicity in the subject as compared to a therapeutically effective amount of a rAAV virion having a wildtype AAV5 VP capsid polypeptide of SEQ ID NO: 1 encapsidating the same payload, wherein the rAAV virion having the wildtype AAV5 VP capsid polypeptide of SEQ ID NO: 1 is administered by the same route of administration.
17. The method or use of any one of the embodiments 12-16, wherein the therapeutically effective amount is at least from 1×10⁵ viral genomes/kg subject weight to 5×10¹⁴ viral genomes/kg subject weight.
18. The method or use of any one of the embodiments 12-17, wherein the rAAV virion is administered via systemic administration.
19. The method or use of embodiment 18, wherein the systemic administration is intravenous administration, intramuscular administration, intraperitoneal administration, or oral administration.
20. The method or use of any one of the embodiments 12-19, wherein the subject is a human or a non-human animal.
21. The method or use of any one of the embodiments 12-20, wherein the subject has a disease.
22. The method or use of embodiment 21, wherein the disease is a neurological condition.
23. The method of embodiment 22, wherein the neurological condition is AADC deficiency, Alzheimer's Disease, tauopathies, synucleinopathies, Batten Disease, MPS-IIIB, frontotemporal dementia with GBA1 mutations (PD-GBA), neuronpathic Gaucher's Disease, Gaucher Disease Type 2, Canavan Disease, Parkinson's Disease, Tay-Sachs Disease, Huntington's Disease, Protocki-Lupski Syndrome, amyotrophic lateral sclerosis, down syndrome, Sanfilippo Disease Type A, Sanfilippo Disease Type B, or Rett syndrome.
24. The method of embodiment 23, wherein the dementia is frontotemporal dementia (FTD).

### Series W Embodiments

1. The method or use of any embodiment disclosed herein, wherein the disease is selected from Table 1.
2. The method or use of any embodiment disclosed herein, wherein the rAAV VP1 capsid at position 587 (Xaa7) is not A, C, D, E, F, G, H, I, K, M, P, Q, R, V, W, or Y.
3. The method or use of any embodiment disclosed herein, wherein the rAAV VP1 capsid at position 587 can be N, S, or T.
4. The method or use of any embodiment disclosed herein, wherein the rAAV VP1 capsid at position 582 (Xaa2) is not G, V, L, or I.
5. The method of use of any embodiment disclosed herein, wherein the rAAV VP1 capsid comprises one or more mutations outside of the 581-589 (Xaa1-Xaa9) region wherein the rAAV VP1 capsid is capable of assembling and exhibiting a desired tissue tropism.

### 1.2. EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### Example 1

### High Throughput Capsid Engineering Systems

This example describes the high throughput capsid engineering systems and methods disclosed herein to discover engineered tissue tropic AAV variants. The high throughput capsid engineering system is schematized in FIG. 1. As shown in FIG. 1, in the pre-assembly phase, processes described herein allow for generation of plasmid libraries containing variants of the AAV capsid gene with massive diversity at specific locations in the capsid gene encoding sequence. This library of variants is assembled into virus using the 2-plasmid system described herein, then assembled viruses are directly injected into non-human primates (NHP) for in vivo selection. Tissues are harvested and tissue-transducing capsid genes are labeled with unique molecular identifiers and barcodes. These variant sequences/UMIs/barcodes are parameterized and machine learning algorithms are used to identify deterministic features of specific tissue targeting/de-targeting capsids. “De-targeting” may be referred to herein as “sparing” with respect to a particular tissue. For example, liver detargeting or liver sparing may both be used herein to refer to variants or properties of variants that preferentially exhibit reduced homing to liver tissue.

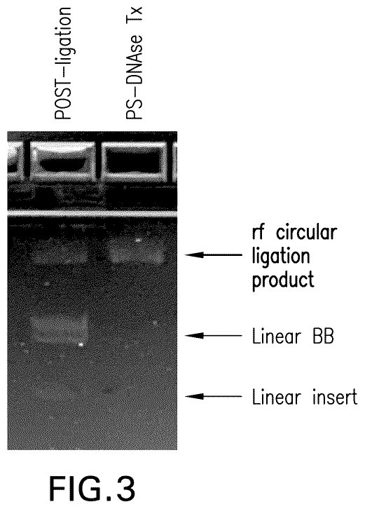

Processes described herein allow for cloning of synthetic oligonucleotide variants of the AAV capsid gene with massive diversity. This library of variants is assembled into virus using the 2-plasmid system described herein, then assembled viruses are directly injected into non-human primates for in vivo selection. The process was carried out as follows, but it is understood that reasonable and scientifically standard modifications to these methods are encompassed herein. The library template was primary PCR amplified (8-10 cycles) and DNA is purified using a column (pool library). Optionally, a second 8 cycle amplification is performed from the library pool. DNA is purified using a suitable column. Next, a digestion was performed with NEB BsaI HF-V2 (library—3 hours; backbone—2 hours+1 hour with calf intestinal alkaline phosphatase) and DNA is purified using a column. The resulting digest was combined at a 3:1 ratio and ligated with NEB T4 ligase ON a 16° C., and more ATP (1 μM) and T4 ligase was added the following morning. A “ligase cycle” was carried out 20 times. Ligase cycling refers to 10 seconds at 16 degrees C., followed by 30 seconds at 25 degrees C. for each “cycle”. The ligase was heat inactivated and more ATP (1 μM) was added. PS-DNAse was added for 2 hours at 37° C. to digest linear (un-ligated) species, enriching for relaxed form (rf) circular DNA (FIG. 3). DNA was purified using a column and DNA was eluted in water. Electrocompetent recombinase-defective bacteria was transformed at massive scale by performing many (>100) electroporation reactions to incorporate library plasmids. Transformed bacteria was selected using antibiotic selection in liquid shaking culture overnight at 37° C. Transformation efficiency was confirmed and library coverage was measured by plating limiting dilutions of transformed bacteria on antibiotic selective agar plates. Library plasmid DNA was prepared for sequencing and transfection for viral production using a Megaprep kit. Virions were produced via “double transfection”: viral producer eukaryotic cell lines (e.g., HEK293) were co-transfected with a mixture of Adenovirus-derived “helper” gene plasmid and library plasmids. Virus from cells/supernatant was purified the following 3-7 days using density centrifugation or FPLC methods. DNA from virions was amplified for NGS sequencing analysis of assembly. Optionally, cloning may be repeated with assembly-competent variants. Purified library virus was intravenously administered to NHPs at a dose range between 1-5×10¹³ viral genomes/kg. Tissues were harvested after 4-6 weeks and library variants were sequenced.

Tissues were harvested and transducing capsid genes are labeled with unique molecular identifiers and barcodes. These variant sequences/UMIs/barcodes were parameterized, and machine learning algorithms were used to identify deterministic features of specific tissue targeting/de-targeting capsids.

FIG. 2A provides a side view (top panel) and top view (bottom panel) of key residues of known AAV capsids that have been shown to interact with target cells. FIG. 2B illustrates salient features of the AAV capsid library described in Example 1, showing the region of introduced diversity, with residue numbering corresponding to the numbering of amino acids in AAV5 VP1. The library plasmid includes the invariant rep and diversified cap coding sequences between AAV ITRs (a cis-packaging approach).

As noted above, Plasmid-Safe DNAse (PS-DNAse) was used to remove linear DNA after ligation. Use of this linear-selective DNAse enzyme to degrade un-ligated DNA maximizes transformation efficiency to maximize library diversity. FIG. 3 is a photograph of a gel showing effective removal of the linear insert and linear plasmid backbone (“BB”) following digestion with PS-DNAse. Total efficiency of plasmid library generation was 4.23×10⁹ colony-forming units per μg plasmid DNA in one example of this method.

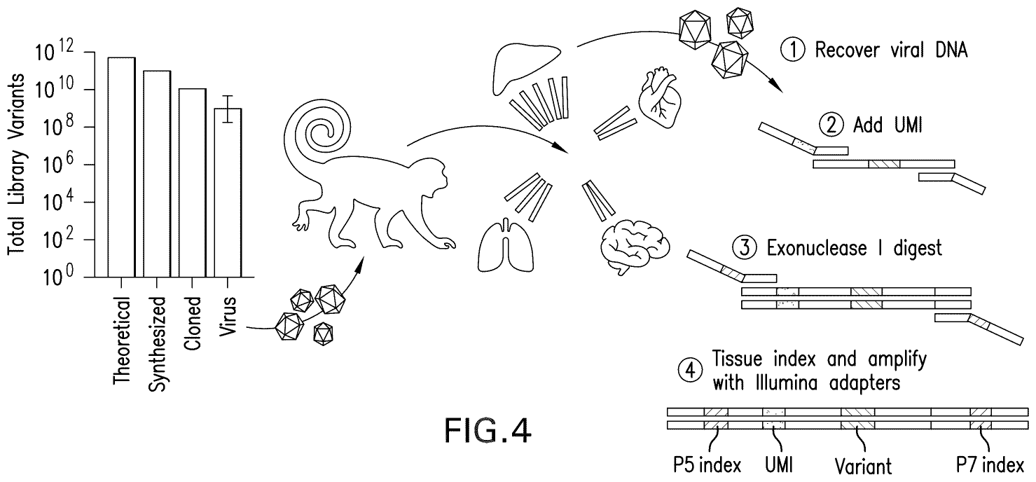

FIG. 4 is a schematic of the high throughput AAV capsid engineering system described in EXAMPLE 1. The histogram shows the size of the AAV capsid library at various stages of preparation, tracking the size of the library from theoretical diversity, synthesized capsid genes, total cloned variants, to sequenced assembled viruses (error bars denote minimum/maximum predicted diversity from NGS sequencing analysis). The pooled viral library was injected into non-human primates and following a period of time sufficient to ensure stable transduction, the animals were euthanized, and tissues were harvested. DNA was purified from the tissues (1), and (2) a unique molecular identifier (UMI) was appended. Exonuclease I was added to digest excess UMI-containing primers (3). Subsequent PCR amplification added sequencing indexes/tissue barcoding and adapters for sequencing. The addition of UMIs allows for high resolution frequency analysis of capsid variants in the tissues.

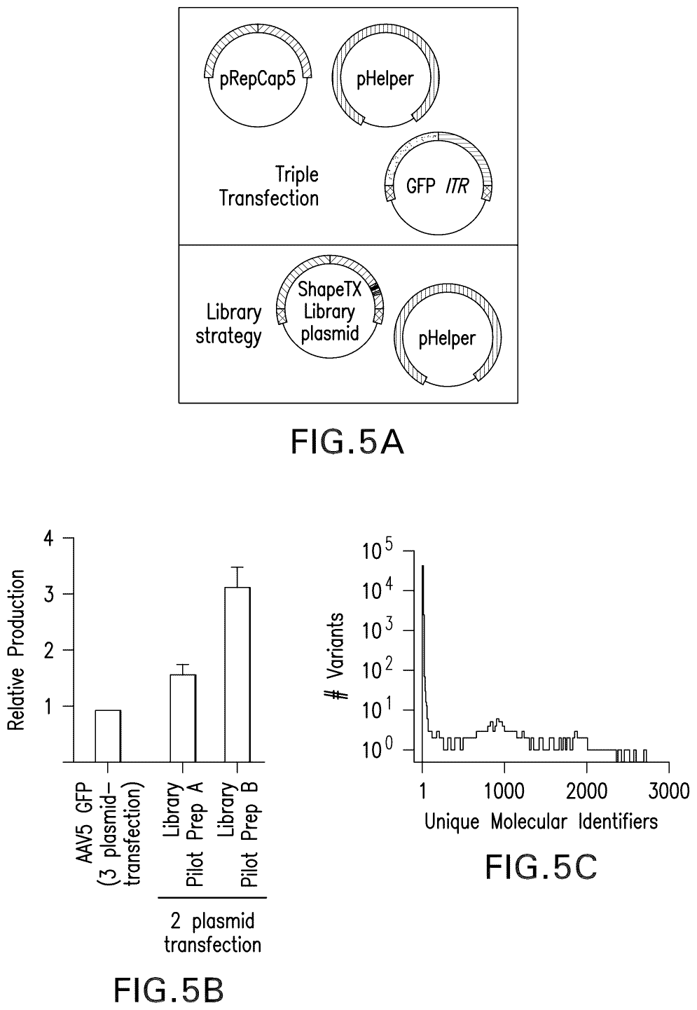

FIG. 5A illustrates the packaging approach used to maximize rAAV production from the library, with FIG. 5A comparing standard triple transfection (top panel) to the two-plasmid cis packaging approach used in EXAMPLE 1 (bottom panel), and FIG. 5B showing relative production of wild type AAV5 using the two transfection approaches. FIG. 5C is an example of UMI distribution in a liver specimen and shows a majority of capsid variants are found a single time (single UMI). However, a subset of capsid variants is enriched in a given tissue and may have increased numbers of UMIs, corresponding to their increased frequency in the target tissue.

### Example 2

### Identification and Analysis of AAV5 Variants in NHPs

This example describes identification and analysis of tissue targeting AAV5 capsid variants discovered using the general methods described in EXAMPLE 1. To identify variants with preferential infection of specific tissues (tropism), the biodistribution of the AAV5-based variant capsid library was evaluated in non-human primates (NHPs). The biodistribution investigation was performed according to Primate Products, LLC Standard Operating Procedures and authorized veterinary standards. Prior to library dose injection, a pre-study schedule of activities was performed to benchmark NHP clinical pathology, general health, and AAV5 seronegativity. Study animals were selected based on seronegativity for antibodies against AAV5.

NHPs received an IV Bolus injection of the AAV5-based capsid library at a dose range between 1-5×10¹³ viral genomes/kg. The final diluted AAV5-based capsid library sample was mixed and transferred to sterile syringes for intravenous administration with a dose volume of 5 mL/kg. Dose preparations were made using common pipetting and transferring techniques of the AAV5-based capsid library. From a common stock of diluted stock of AAV5-based capsid library, dose volumes were prepared separately for each animal based on their respective body weight in order to deliver equivalent amount of vector to each animal on a per kg basis.

Following Day 1 library injection, clinical observations were made twice a day (Days 1-29) and body weight was checked weekly. At Day 29, all study animals were euthanized and underwent tissue collection in accordance with Primate Products, LLC Standard euthanasia procedures.

A diverse array of tissues were collected, including: adrenal gland, aorta, bone with bone marrow, brain (cerebellum), brain (hippocampus, dentate gyrus), brain (hippocampus, CA1), brain (hippocampus, CA3), brain (hypothalamus), brain (cortex, temporal), brain (cortex, forebrain), brain (cortex, occipital), brain (substantia nigra), brain (thalamus), cecum, colon, duodenum, epididymis, esophagus, eye, gallbladder, heart, ileum, jejunum, kidney, liver, lung, lymph node(s), mammary gland, ovary, pancreas, parathyroid gland, peripheral nerve (sciatic), pituitary, prostate, salivary gland, seminal vesicle, skeletal muscle, skin, spinal cord, spleen, stomach, testis, thymus, thyroid gland, trachea, urinary bladder, uterus, and vagina. Tissues were collected as two 1 cm³ blocks (or whole organ) and preserved in liquid nitrogen. Liver was specifically collected in five 1 cm³ blocks. For Brain, two 1 cm³ blocks were collected from each region as noted in the above list, with the exception of smaller regions, which were collected in their entirety.

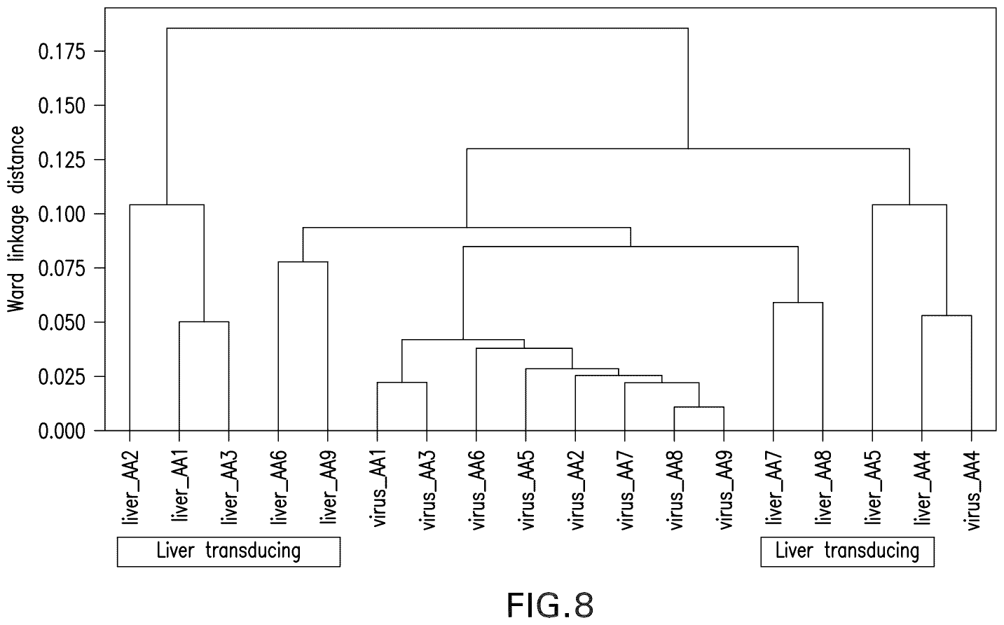

FIG. 8 is a schematic of machine learning-based clustering of capsid variants. The example utilizes capsid variant sequences upon which machine learning algorithms were used to map the similarity of capsids among those that infected the liver. This output can then be used to inform selection of candidate variants to selectively target the tissue of interest.

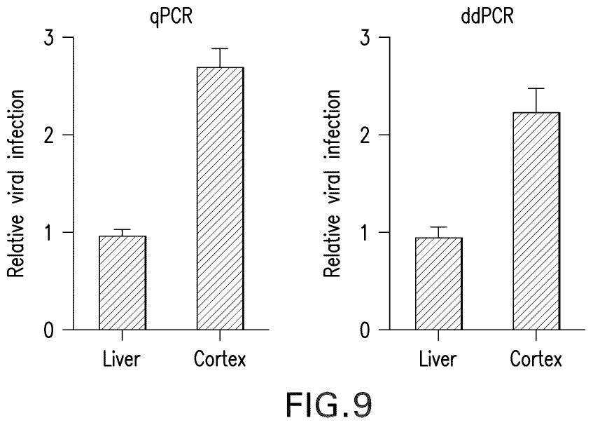

FIG. 9 illustrates an example of the performance of the library as a whole infecting cortex at a higher relative level than liver after the intravenous administration to an NHP. DNA was isolated from the liver and cortex and either qPCR (at left) or droplet digital PCR (ddPCR) (at right) were used to quantify viral genomes recovered from the respective tissues. By these methods of quantification, the library as a whole has ˜2-fold increased CNS targeting over the liver.

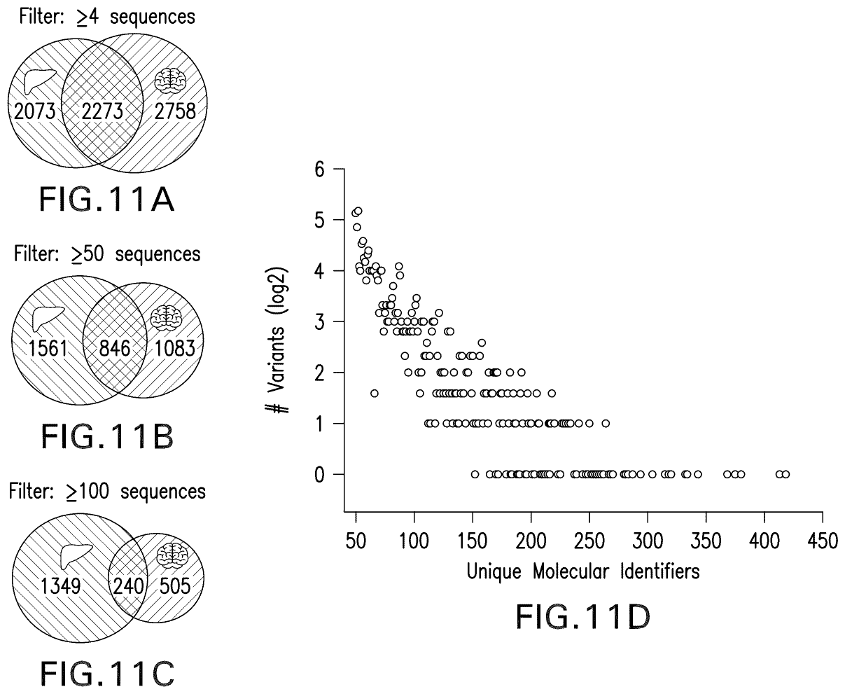

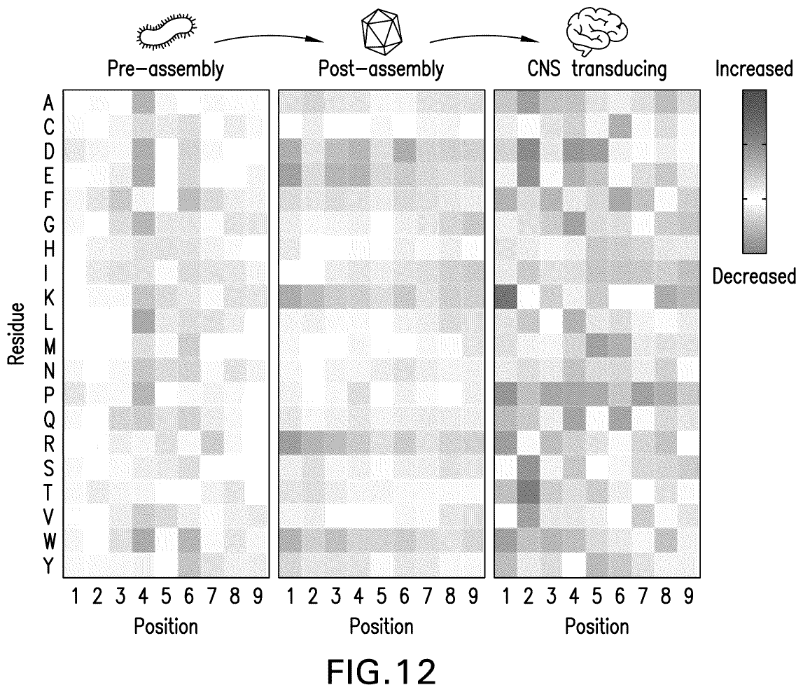

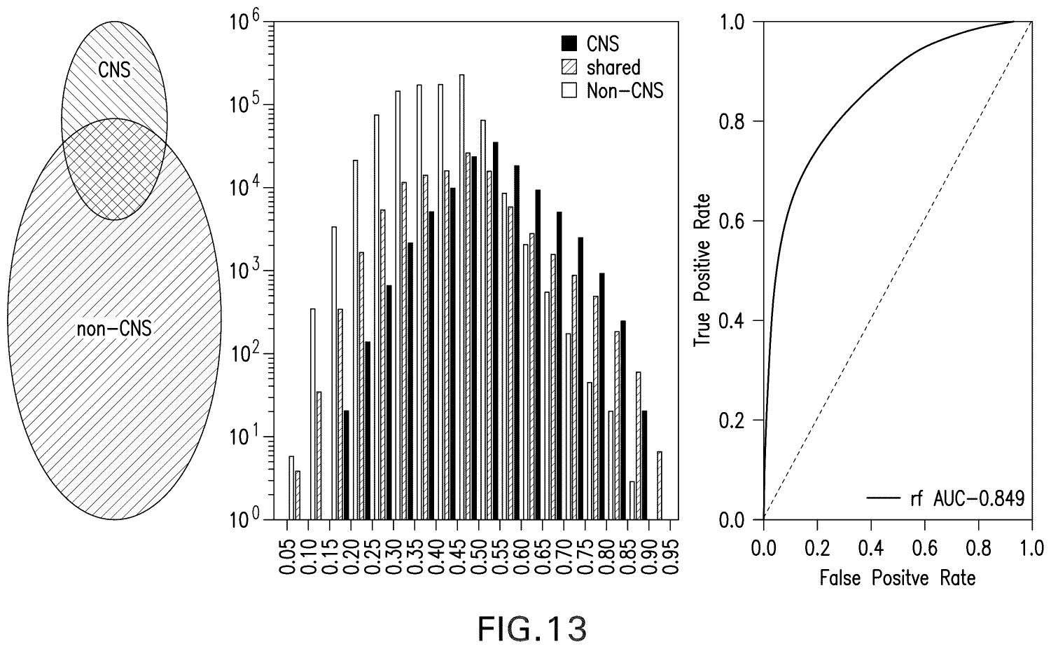

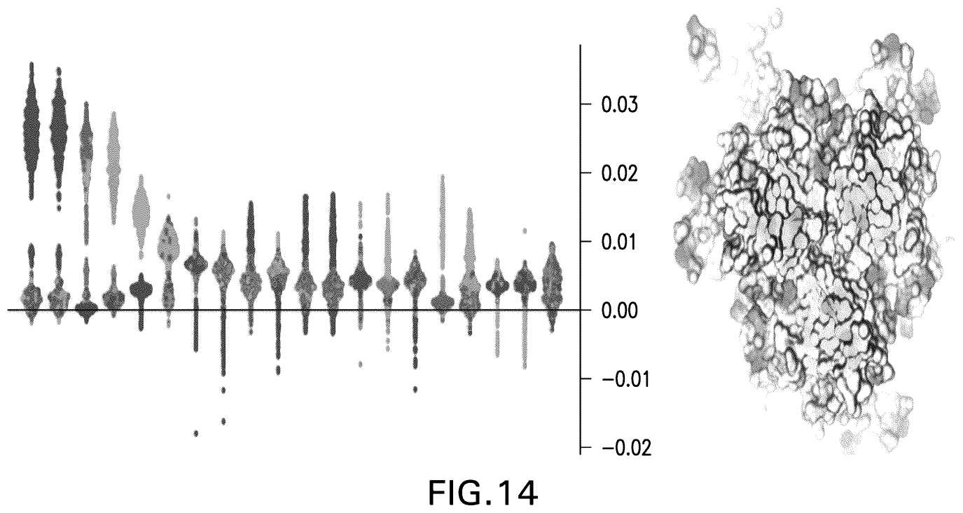

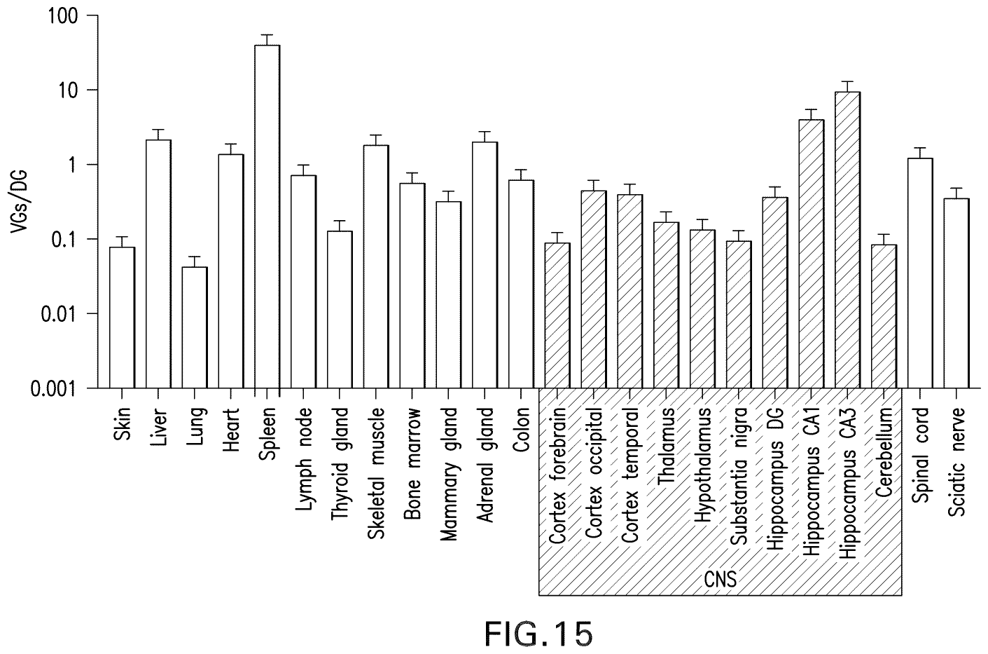

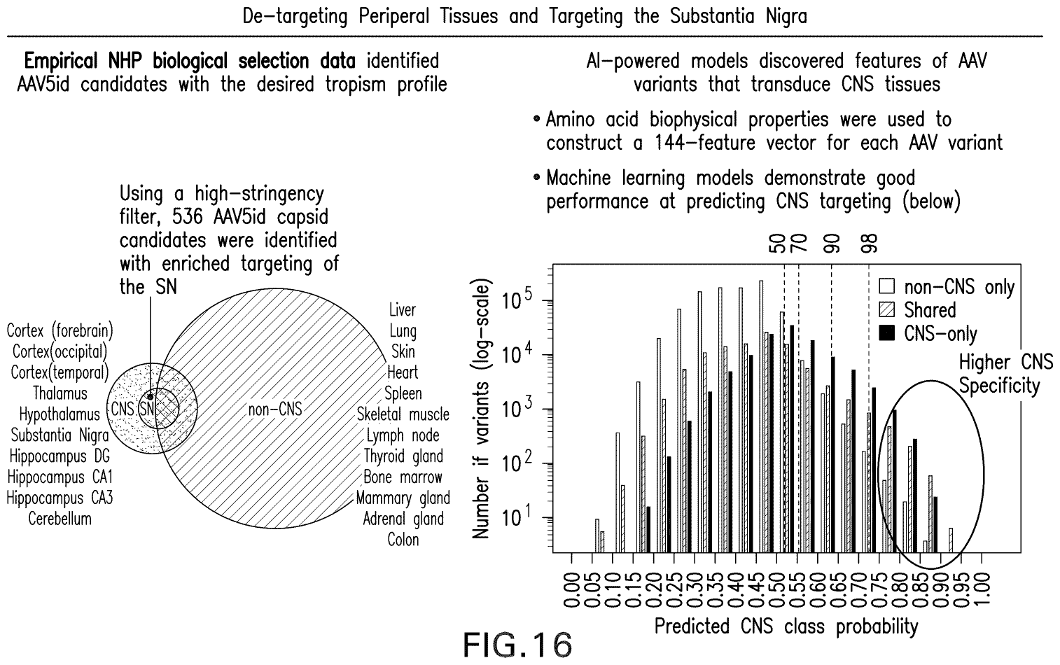

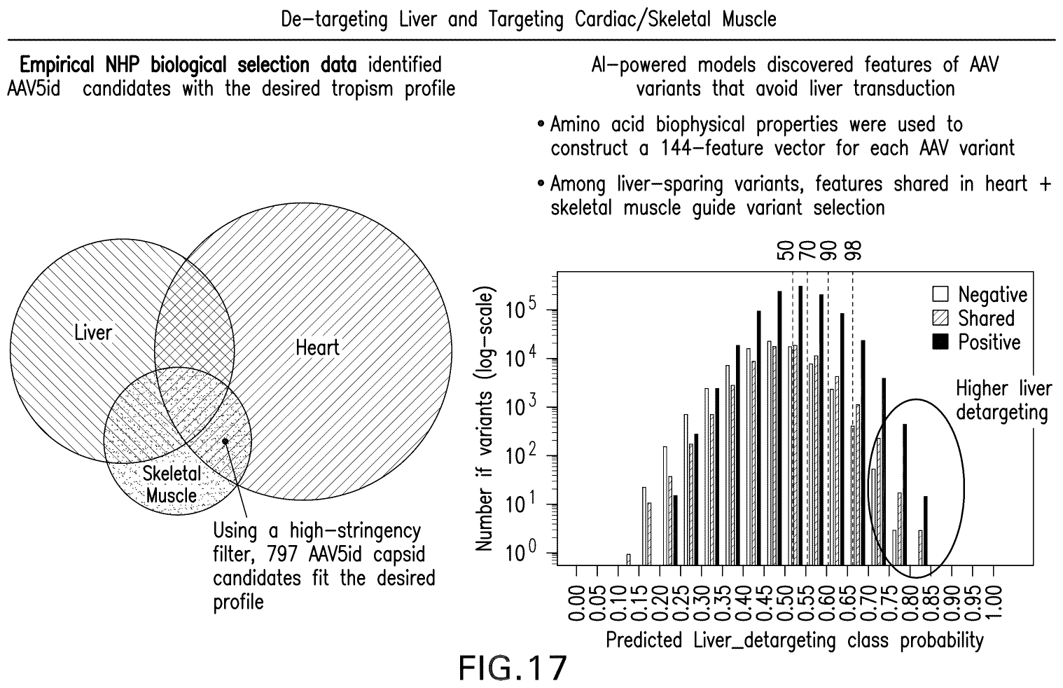

FIGS. 11A-D show Venn diagrams and analytic tables depicting the number of unique sequence variants found in liver tissue only, liver and cortex tissues, and cortex tissue only for three different sequencing analysis filters. Next-generation sequencing was performed on viral genomic capsid variants recovered from liver or cortical tissue samples. Unique Molecular Identifiers (UMIs) were appended as part of the molecular recovery process as described herein. This allows for increased confidence that a given capsid variant is present in the tissue, and may be correlated to abundance. FIG. 11A shows a Venn diagram in which the sequencing analysis filter applied was 4 or greater distinct UMIs—also referred to herein as “count”—per each distinct capsid sequence variant. FIG. 11B shows a Venn diagram in which the sequencing analysis filter applied was 50 or greater UMIs. FIG. 11C shows a Venn diagram in which the sequencing analysis filter applied was 100 or greater distinct UMIs. FIG. 11D shows a histogram analysis of the distribution of UMIs in the population of capsid sequence variants found in cortex only in which the sequencing analysis filter applied was 50 or greater UMIs as shown in FIG. 11B.

### Example 3

AAV5 Variants that Functionally Assemble in Virus

This example describes engineered AAV5 variants that assemble into virions discovered using the methods and systems described in EXAMPLE 1-EXAMPLE 2. Following production and purification of the library virus as described in EXAMPLE 1, assembly of the AAV5 library virus was assessed. The composition of capsid variants was measured by NGS of an amplicon spanning the variant region. The frequency of a given amino acid residue occurring at a specified position corresponding to position 581 to position 589 of SEQ ID NO: 1 (generalized in SEQ ID NO: 2) over the frequency of that given amino acid residue occurring at the specified position in the library plasmid was analyzed to identify sequence rules that are preferred for viral assembly. With reference to TABLE 3 below, and SEQ ID NO: 2, the following amino acids can be independently mutated, in any combination, at any one or more positions Xaa1-Xaa9, to provide an AAV VP1 capsid that is capable of assembling. Additionally, one or more mutations outside of the Xaa1-Xaa9 region can be allowed, as long as the capsid is still capable of assembling.

TABLE 3
Viral Assembly Options

Xaa1 is A, D, E, G, L, M, N, Q, S, T, or V
Xaa1 is A, D, E, M, or T
Xaa1 is E
Xaa2 is A, C, D, E, G, H, I, N, P, Q, S, T, or V
Xaa2 is A, S, T, or V
Xaa2 is A
Xaa3 is A, D, E, G, H, M, N, Q, S, T, or V
Xaa3 is D, E, N, Q or T
Xaa3 is D or T
Xaa4 is A, D, E, G, H, N, P, Q, S, or T
Xaa4 is D, E, P, or Q
Xaa4 is E
Xaa5 is A, C, D, E, G, H, N, Q, S, T, or Y
Xaa5 is D, E, N, Q or T
Xaa5 is N.
Xaa6 is A, D, E, G, H, N, P, Q, S, or T;
Xaa6 is D, N, or Q; or
Xaa6 is D.
Xaa7 is s A, C, D, E, G, H, N, Q, S, or T;
Xaa7 is A, D, E or G; or
Xaa7 is A.
Xaa8 is A, C, D, E, G, H, N, Q, S, or T;
Xaa8 is A, D, G, or S; or
Xaa8 is G.
Xaa9 is A, D, E, G, H, N, P, Q, S, or T;
Xaa9 is A, D, G, or P; or
Xaa9 is G.

### Example 4

AAV5 Variants with Liver Tissue Targeting and AAV5 Variants with Liver-Detargeting Properties

This example describes engineered AAV5 variants with tissue tropism in liver and AAV5 variants that preferentially detarget liver tissue that were discovered using the methods and systems described in EXAMPLE 1-EXAMPLE 2.

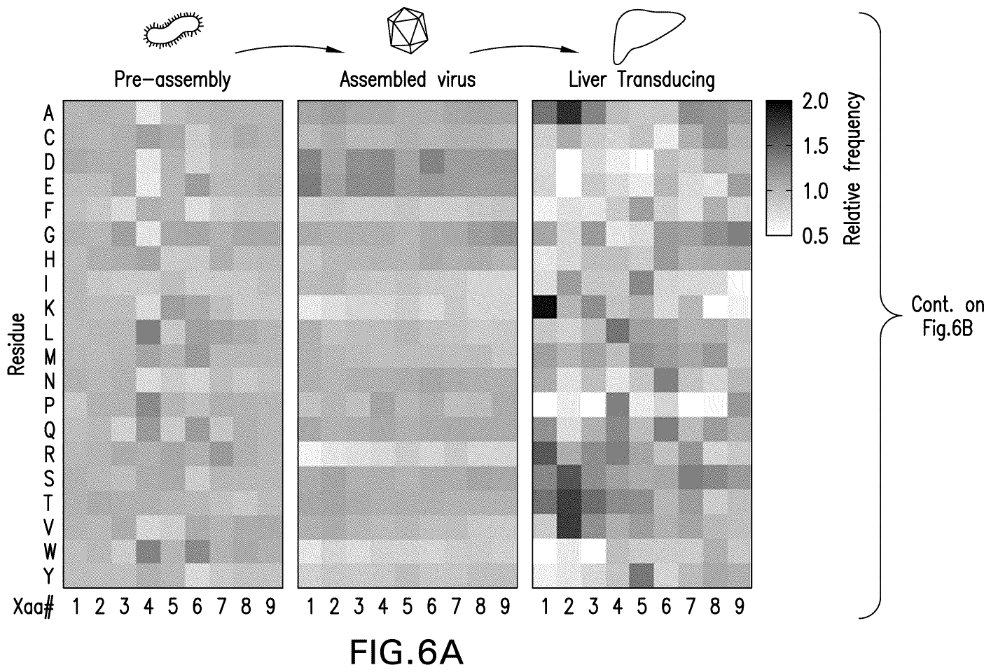

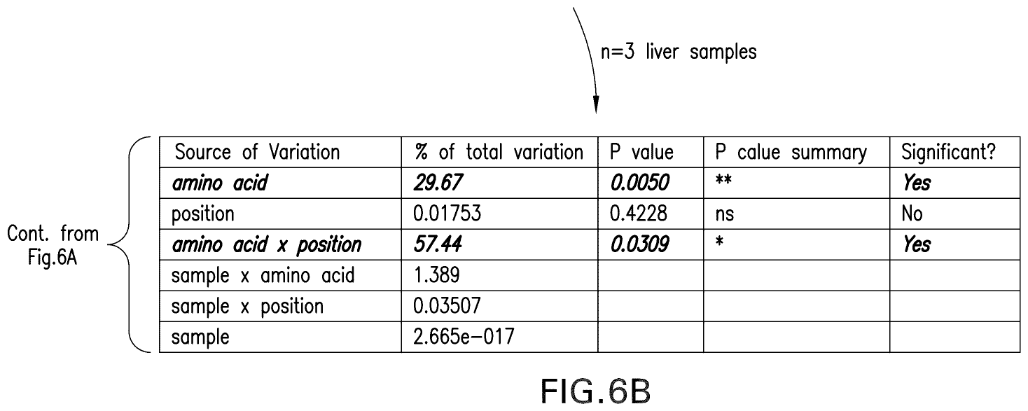

FIGS. 6A-B are heat maps and a statistical analysis showing clear functional selection through viral assembly and tissue transduction in liver. FIG. 6A is a heat map prepared at various stages of the high throughput system described in EXAMPLES 1-2 from pre-assembly, assembled virus, to liver transduction. Thus, FIG. 6A, at right, shows a heatmap of favored positional residues for all liver transducing variants divided by all assembled virus. For the pre-assembly heat map, the library of capsid variants was cloned into plasmids and transformed into electrocompetent bacteria. The resultant library was sequenced and amino acid diversity was measured at each position, with the low levels of positional variation likely arising from synthesis error. Subsequent assembly into virus showed clear amino acid residue/positional biases that highlight selection for viral assembly. Liver transducing viruses showed even greater patterns of AA residue/positional selection, with distinctly favored/disfavored variants. Each of these heatmaps was normalized by their respective input frequencies. FIG. 6B is a table of statistical analysis (ANOVA) showing that in three liver samples, amino acid residue distribution and residue-position varied significantly, but inter-sample variation was not significant.

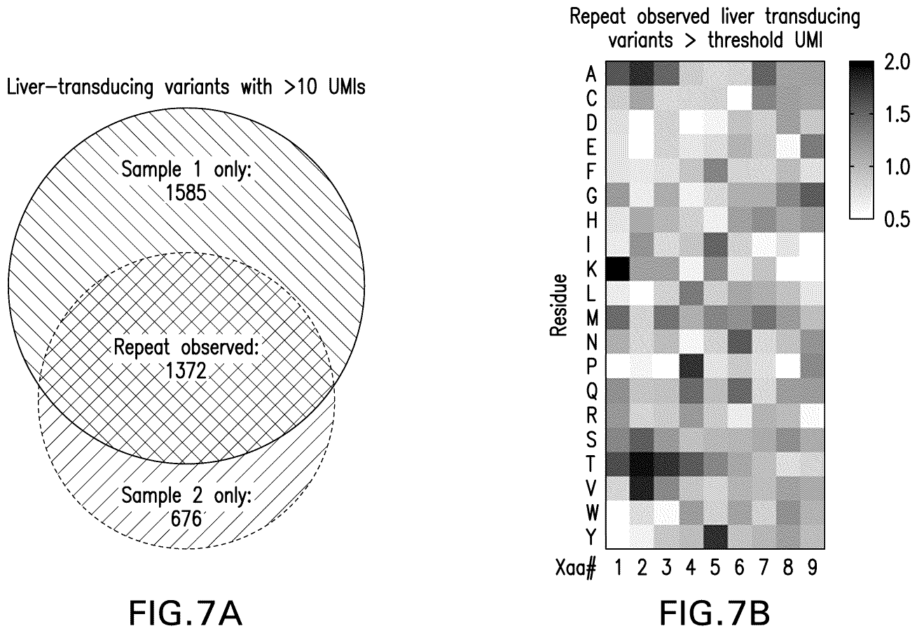

FIGS. 7A-B illustrate analysis of repeat observed (high UMI) capsid variants between multiple samples. FIG. 7A is a Venn diagram illustrating counts of overlapping variant identification in two liver samples: ˜ 38% of variants with >10 UMIs were observed in both samples. FIG. 7B is a heat map showing positional amino acid distribution illustrating the strongly selected residues/positions of repeat observed capsid variants.

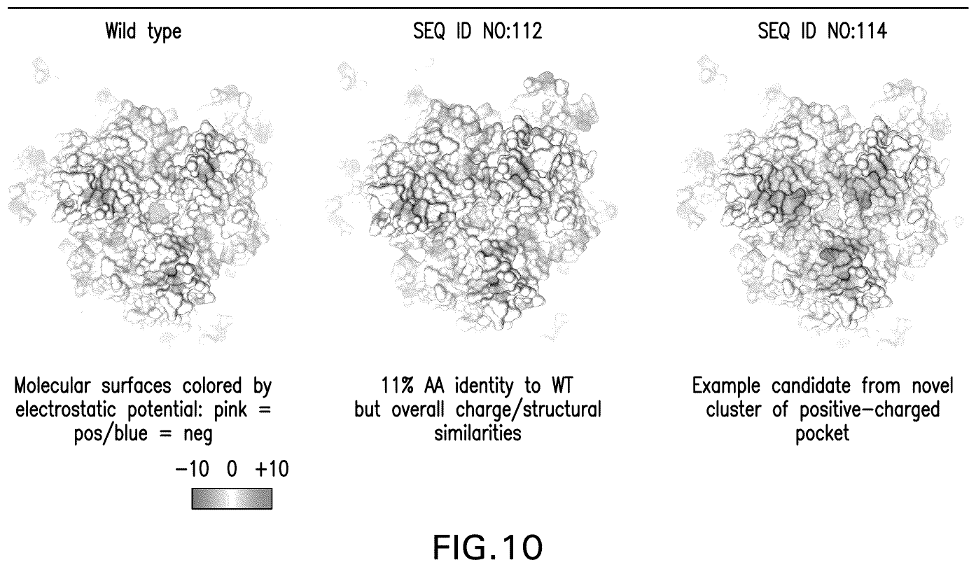

FIG. 10 provides structure illustrations with the illustration on the left representing the crystal structure of wildtype AAV5 [SEQ NO: 1] (PDB: 6JCT from Zhang et al. Nat Commun. 2019 Aug. 21; 10 (1): 3760), oriented to the 3-fold axis of symmetry. The middle and right illustrations represent variants recovered from NHP liver replacing the 581-589 residues with NPAMFNCDY (middle panel, amino acid SEQ ID NO:112), and KLTAHIYAL (right panel, amino acid SEQ ID NO:114). These illustrations were generated using PyMOL to replace the variant sequences and are colored by predicted electrostatic charge to highlight the differences in charge among liver variants recovered from the same tissue.

The frequency of a given amino acid residue occurring at a specified position corresponding to position 581 to position 589 of SEQ ID NO: 1 (generalized in SEQ ID NO: 2) in variants identified in liver over the frequency of that given amino acid residue occurring at the specified position in variants forming assembled virus was analyzed to identify a set of sequence rules for capsids that preferentially target liver tissue. With reference to TABLE 4 below, and SEQ ID NO: 2 (AAV5 VP1), the following amino acids can be independently mutated, in any combination, at any one or more positions Xaa1-Xaa9, to provide a VP1 capsid with enhanced liver tropism as compared wildtype AAV5 VP1 capsid (SEQ ID NO: 1), where liver tropism here refers to properties that are preferred for liver transduction over properties that are preferred for virion assembly. Additionally, one or more mutations outside of the Xaa1-Xaa9 region can be allowed, as long as the capsid is still capable of assembling and exhibiting the desired liver targeting property.

TABLE 4
Liver Tropism Options and Rules

Xaa1 is A, G, K, M, N, Q, R, S, or T;
Xaa1 is A, K, M, or T; or
Xaa1 is K.
Xaa2 is A, C, H, I, K, S, T, or V;
Xaa2 is A, S, T, or V; or
Xaa2 is T.
Xaa3 is A, G, H, K, M, N, Q, R, S, T, or V;
Xaa3 is A, M, or T; or
Xaa3 is s A or T.
Xaa4 is L, M, P, Q, R, T, or W;
Xaa4 is L, P, Q, or T; or
Xaa4 is P.
Xaa5 is F, H, I, K, M, T, or Y;
Xaa5 is H, I, or Y; or
Xaa5 is Y.
Xaa6 is E, G, H, L, M, N, Q, T, or W;
Xaa6 is N, or Q; or
Xaa6 is N.
Xaa7 is A, C, G, H, L, M, R or S;
Xaa7 is A, C, H or M; or
Xaa7 is A.
Xaa8 is A, C, D, F, G, H, M, Q, S, V, W, or Y;
Xaa8 is G, M, Q, or S; or
Xaa8 is G.
Xaa9 is A, C, E, G, H, M, N, P, Q, S, V, or W;
Xaa9 is E, G, or P; or
Xaa9 is G.

The frequency of a given amino acid residue occurring at a specified position corresponding to position 581 to position 589 of SEQ ID NO: 1 (generalized in SEQ ID NO: 2) in variants not identified in liver over the frequency of that given amino acid residue occurring at the specified position in variants forming assembled virus was analyzed to identify a set of sequence rules that are preferred for liver detargeting. With reference to Table 5 below and AAV5 VP1 (SEQ ID NO: 2), the following amino acids can be independently excluded, in any combination, to provide an AAV VP1 capsid that is capable of assembling and is less targeted to liver tissue (“liver-sparing”, or “liver-detargeted”) than the wildtype AAV5 VP1 capsid (SEQ ID NO: 1), where liver-detargeting here refers to properties that are preferred among viruses that have decreased liver transduction over properties that are preferred for virion assembly. Additionally, one or more mutations outside of the Xaa1-Xaa9 region can be allowed, as long as the capsid is still capable of assembling and exhibits the desired features.

TABLE 5
Liver Detargeting Rules

Xaa1 excludes K;
Xaa1 excludes A, K, M, or T; or
Xaa1 excludes A, G, K, M, N, Q, R, S, or T.
Xaa2 excludes T;
Xaa2 excludes A, S, T, or V; or
Xaa2 excludes A, C, H, I, K, S, T, or V.
Xaa3 excludes A or T;
Xaa3 excludes A, M, or T; or
Xaa3 excludes A, G, H, K, M, N, Q, R, S, T, or V.
Xaa4 excludes P;
Xaa4 excludes L, P, Q, or T; or
Xaa4 excludes L, M, P, Q, R, T, or W.
Xaa5 excludes Y;
Xaa5 excludes H, I, or Y; or
Xaa5 excludes F, H, I, K, M, T, or Y.
Xaa6 excludes N;
Xaa6 excludes N, or Q; or
Xaa6 excludes E, G, H, L, M, N, Q, T, or W.
Xaa7 excludes A;
Xaa7 excludes A, C, H or M; or
Xaa7 excludes A, C, G, H, L, M, R or S.
Xaa8 excludes G;
Xaa8 excludes G, M, Q, or S; or
Xaa8 excludes A, C, D, F, G, H, M, Q, S, V, W, or Y.
Xaa9 excludes G;
Xaa9 excludes E, G, or P; or
Xaa9 excludes A, C, E, G, H, M, N, P, Q, S, V, or W.

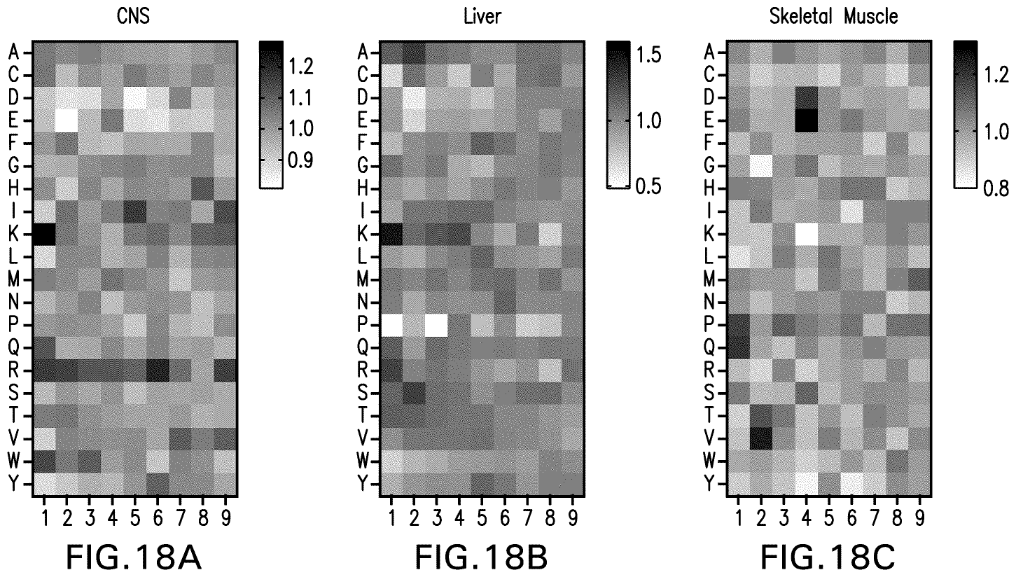

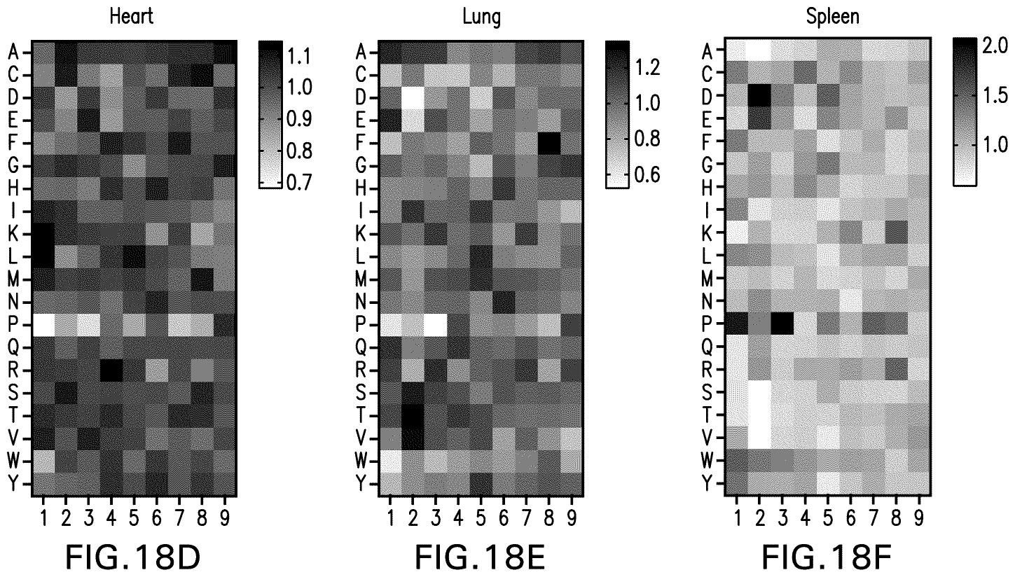

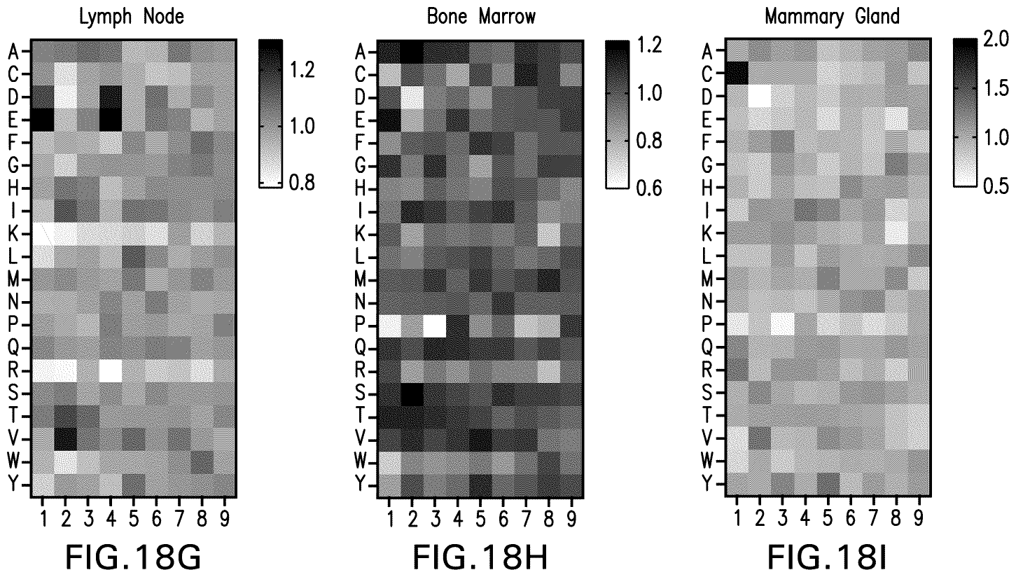

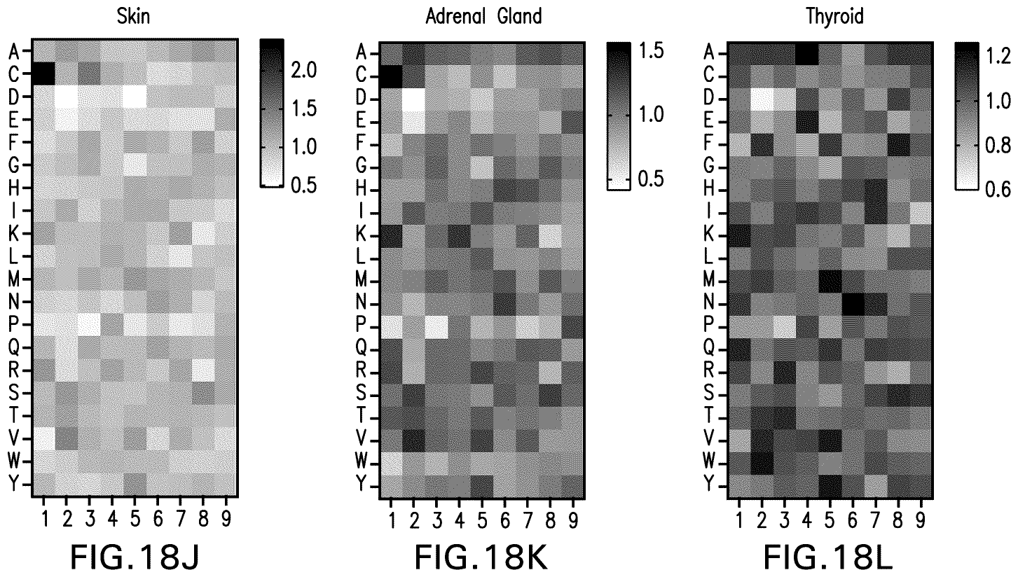

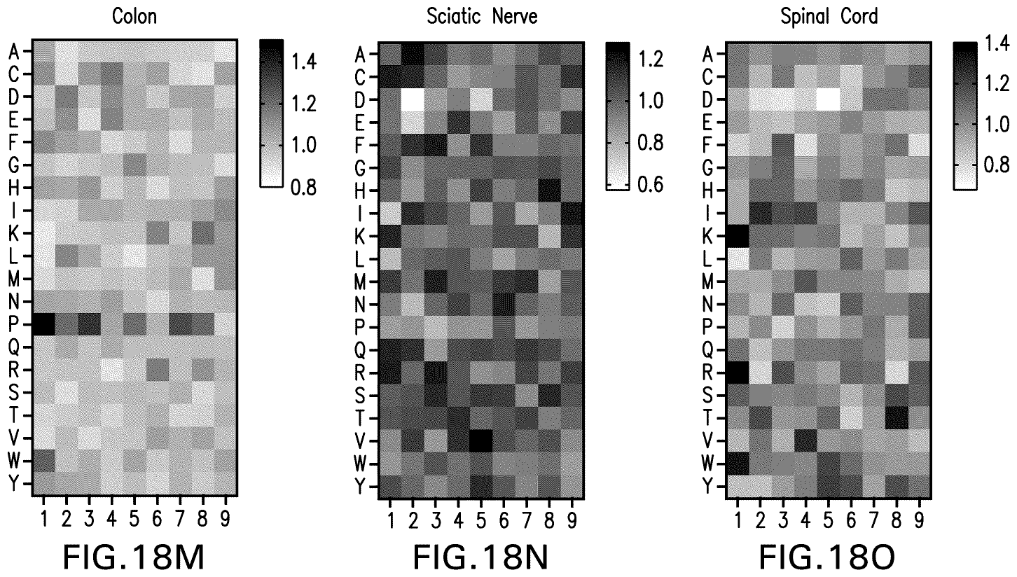

Liver, CNS, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive liver tropism. The results are shown in FIG. 18B which shows the positional frequency of each amino acid in the 581 to 589 region of AAV5 VP1 in liver tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in liver over the frequency of that given amino acid residue occurring at the specified position in variants identified in all other analyzed tissues was compared to identify a set of sequence rules that are preferred for liver targeting. With reference to TABLE 6A below, and SEQ ID NO: 2 (AAV5 VP1), the following amino acids can, thus, be independently mutated, in any combination, at any one or more positions Xaa1-Xaa9, to provide a VP1 capsid with enhanced liver tropism as compared wildtype AAV5 VP1 capsid (SEQ ID NO: 1), where liver tropism here refers to properties that are preferred for liver transduction over properties that are preferred for transduction of all other harvested tissues.

TABLE 6A
Liver Tropism Rules

Xaa1 is selected from A, G, K, M, N, Q, R, S, or T
Xaa1 is selected from A, K, Q, or R
Xaa1 is K
Xaa2 is selected from A, C, I, K, S, T, or V
Xaa2 is selected from A, K, S, or T
Xaa2 is A
Xaa3 is selected from A, G, I, K, M, Q, R, S, T, or V
Xaa3 is selected from A, K, Q, S, or T
Xaa3 is selected from K, Q, or T
Xaa3 is K
Xaa4 is selected from A, I, K, L, P, Q, R, S, T, or V
Xaa4 is selected from K, I, S, or V
Xaa4 is K
Xaa5 is selected from F, I, L, M, T, V, or Y.
Xaa5 is selected from F, L, or Y
Xaa5 is F
Xaa6 is selected from F, H, M, N, Q, S, or Y
Xaa6 is selected from M or N
Xaa6 is N
Xaa7 is selected from A, C, K, M, Q or S
Xaa7 is selected from A, C, or S
Xaa7 is S
Xaa8 is selected from A, C, F, G, M, Q, or S
Xaa8 is selected from A, C, M, or S
Xaa8 is C
Xaa9 is selected from E, F, L, Q, R, or Y
Xaa9 is selected from L, Q, or R
Xaa9 is R

With reference to TABLE 6B below, and SEQ ID NO: 2 (AAV5 VP1), the following amino acids can, thus, be independently mutated, in any combination, at any one or more positions Xaa1-Xaa9, to provide a VP1 capsid with reduced liver tropism as compared wildtype AAV5 VP1 capsid (SEQ ID NO: 1), where liver tropism here refers to properties that are deterministic for liver transduction over properties that are deterministic for transduction of all other harvested tissues.

TABLE 6B

Liver Detargeting Rules
Xaa1 is not selected from A, G, K, M, N, Q, R, S, or T
Xaa1 is not selected from A, K, Q, or R
Xaa1 is not K
Xaa2 is not selected from A, C, I, K, S, T, or V
Xaa2 is not selected from A, K, S, or T
Xaa2 is not A
Xaa3 is not selected from A, G, I, K, M, Q, R, S, T, or V
Xaa3 is not selected from A, K, Q, S, or T
Xaa3 is not selected from K, Q, or T
Xaa3 is not K
Xaa4 is not selected from A, I, K, L, P, Q, R, S, T, or V
Xaa4 is not selected from K, I, S, or V
Xaa4 is not K
Xaa5 is not selected from F, I, L, M, T, V, or Y.
Xaa5 is not selected from F, L, or Y
Xaa5 is not F
Xaa6 is not selected from F, H, M, N, Q, S, or Y
Xaa6 is not selected from M or N
Xaa6 is not N
Xaa7 is not selected from A, C, K, M, Q or S
Xaa7 is not selected from A, C, or S
Xaa7 is not S
Xaa8 is not selected from A, C, F, G, M, Q, or S
Xaa8 is not selected from A, C, M, or S
Xaa8 is not C
Xaa9 is not selected from E, F, L, Q, R, or Y
Xaa9 is not selected from L, Q, or R
Xaa9 is not R

TABLE 7 below provides sequences of the 581 to 589 region for variant AAV VP1 capsid polypeptides that were found in liver tissue and comport to one or more of the rules provided in TABLE 6. The present disclosure, thus, provides for rAAVs composed of engineered AAV5 VP1 capsid polypeptides having a SEQ ID NO: 2, wherein the Xaa1 to Xaa9 region is any one of SEQ ID NO: 43438-SEQ ID NO: 44437, as disclosed in TABLE 7. Also encompassed herein are rAAVs composed of engineered AAV5 VP2 capsid polypeptides and engineered AAV5 VP3 capsid polypeptides having the sequences disclosed in the below table at the regions in AAV5 VP2 (amino acid residues 445 to 453) and AAV5 VP3 (amino acid residues 389-397) corresponding to the amino acids in the AAV5 VP1 581 to 589 region.

TABLE 7
Sequences of the 581 to 589 Region in
AAV5 VP1 Capsid Polypeptide that
Drive Liver Tissue Tropism

| SEQ | |
|---|---|
| ID | 581-589 |
| NO | Sequence |
| 43438 | KADPATSIG |
| 43439 | KAEAMRNDR |
| 43440 | KAECYYGYE |
| 43441 | KAEGENVAK |
| 43442 | KAFPAPQGP |
| 43443 | KAGCMFWCE |
| 43444 | KAQHDTAVG |
| 43445 | KARVNCHGS |
| 43446 | KASDLNTQP |
| 43447 | KCCEYCDIA |
| 43448 | KCCTCVSFP |
| 43449 | KCDFWMGWM |
| 43450 | KCICSDDLK |
| 43451 | KCLSESFWG |
| 43452 | KCLWPFDWG |
| 43453 | KCLWPFNWG |
| 43454 | KCMDAKEHA |
| 43455 | KCQPCIVKC |
| 43456 | KCQSKDTHF |
| 43457 | KCSTDKAVE |
| 43458 | KCVVLENTQ |
| 43459 | KDHWECAGY |
| 43460 | KDNCVMWHW |
| 43461 | KDPKEQFFQ |
| 43462 | KDQQDQCND |
| 43463 | KEDERKSGT |
| 43464 | KETCTNMDL |
| 43465 | KFDLCQDSM |
| 43466 | KFWMPEMEF |
| 43467 | KGDLLDCLE |
| 43468 | KGEITPSGD |
| 43469 | KGGDGNYPF |
| 43470 | KGLDVHALN |
| 43471 | KGWKPDMTN |
| 43472 | KHAAYNACE |
| 43473 | KHQDIMHRG |
| 43474 | KIAQVDTWV |
| 43475 | KIDSQQSNY |
| 43476 | KIDSYGTSM |
| 43477 | KIEMQYRQS |
| 43478 | KIGYWWPQA |
| 43479 | KITPPLGNY |
| 43480 | KIYPECHYS |
| 43481 | KLEPNMADR |
| 43482 | KLLIHGPYE |
| 43483 | KMCGNCEAG |
| 43484 | KMEHMQHQS |
| 43485 | KMESRQLYN |
| 43486 | KMQHYDAEA |
| 43487 | KNDEYRMQN |
| 43488 | KNMLGDWPN |
| 43489 | KNMYYMYEA |
| 43490 | KPATQLDCA |
| 43491 | KPCKHMGGR |
| 43492 | KPDHMRWAL |
| 43493 | KPDQGGICK |
| 43494 | KPEGCLNSR |
| 43495 | KPFQALMMS |
| 43496 | KPGVHSHHG |
| 43497 | KPIAETVNW |
| 43498 | KPMGCHASS |
| 43499 | KPTPHAYDA |
| 43500 | KPWTQIASH |
| 43501 | KPWYERYQT |
| 43502 | KQATYPHAW |
| 43503 | KQDTKNLDE |
| 43504 | KQEMFGRFL |
| 43505 | KQIPFVSGD |
| 43506 | KQQMDHCAM |
| 43507 | KQTPYEMFH |
| 43508 | KRWVAEWIV |
| 43509 | KSDCQFRDH |
| 43510 | KSDRWAWGC |
| 43511 | KSELSTWGI |
| 43512 | KSEPPQVVT |
| 43513 | KSGYIDLYH |
| 43514 | KSIFLDSWE |
| 43515 | KSILKEDDG |
| 43516 | KSNGDMSGL |
| 43517 | KSNRDMNFA |
| 43518 | KSQMEMCIG |
| 43519 | KSRDNDGVL |
| 43520 | KSTTDSGCQ |
| 43521 | KSVQVPDAM |
| 43522 | KSWINEWLS |
| 43523 | KTDGPGTSW |
| 43524 | KTKPLDTVD |
| 43525 | KTLSDLAVQ |
| 43526 | KTLWALSCN |
| 43527 | KTNAEENFI |
| 43528 | KTNAWDTPC |
| 43529 | KTNPVKYVK |
| 43530 | KTQCTAQQN |
| 43531 | KTQNEKVWL |
| 43532 | KTQTPCFQH |
| 43533 | KTSWMMEAV |
| 43534 | KTTDGKMID |
| 43535 | KTTQWHEWA |
| 43536 | KVAQGLGPE |
| 43537 | KVCEIGTQG |
| 43538 | KVDAFEGRM |
| 43539 | KVECAQFDV |
| 43540 | KVEPSMLLD |
| 43541 | KVGINELFT |
| 43542 | KVHLSLWPQ |
| 43543 | KVIMYQHNG |
| 43544 | KVMWAGSKR |
| 43545 | KVNADGSRC |
| 43546 | KVPFACCSS |
| 43547 | KVRNKDPTM |
| 43548 | KVSVQGQVF |
| 43549 | KVTHCEAMN |
| 43550 | KWELYNNDP |
| 43551 | KWHTEDRNC |
| 43552 | KYNYKTYKT |
| 43553 | KACSAMDFC |
| 43554 | KALHQINED |
| 43555 | KAPMGWMNE |
| 43556 | KAQCALDTM |
| 43557 | KCENKDVGY |
| 43558 | KCNMNQEAE |
| 43559 | KCPQLNDNQ |
| 43560 | KCQQEQQYN |
| 43561 | KEGVENTGW |
| 43562 | KEMWNCDPA |
| 43563 | KETPFLMQS |
| 43564 | KFHVKPVDM |
| 43565 | KHMQSDHFT |
| 43566 | KHVYSEMGL |
| 43567 | KIHKNEDLF |
| 43568 | KKLDGHDKY |
| 43569 | KKVMGMGIY |
| 43570 | KLVQRCEVD |
| 43571 | KMLDWKNCS |
| 43572 | KNGVDDAHN |
| 43573 | KNVVHEKQA |
| 43574 | KQALMENFF |
| 43575 | KQTTAEIGY |
| 43576 | KRDDCHNDN |
| 43577 | KRTVIHEMM |
| 43578 | KSHYGAGVT |
| 43579 | KSIDNYLCE |
| 43580 | KSKRWNWDV |
| 43581 | KSVCCQINN |
| 43582 | KTFCTQYMD |
| 43583 | KTFLVKTFI |
| 43584 | KTRNCPKEA |
| 43585 | KTSLVVRRG |
| 43586 | KTTLCVQWG |
| 43587 | KTVQQVQMG |
| 43588 | KTWWTEQSA |
| 43589 | KVAADLGHL |
| 43590 | KVEHYYDIG |
| 43591 | KVISHCDHY |
| 43592 | KWRVSVYVM |
| 43593 | KYEEFECMY |
| 43594 | KYICTRWHC |
| 43595 | KYSPTPFMY |
| 43596 | KYTPWDATF |
| 43597 | IACRCNCFD |
| 43598 | IADSKRLQK |
| 43599 | IADVQRCHY |
| 43600 | IAGHYDSWK |
| 43601 | IAGMKVITP |
| 43602 | IAHSVFTDH |
| 43603 | IAQRTRQQQ |
| 43604 | IAQTTLLPG |
| 43605 | IATIKVHAS |
| 43606 | LAAPKTMCH |
| 43607 | LACFSYTNV |
| 43608 | LAEPKKWHT |
| 43609 | LAFQYCYPP |
| 43610 | LAGLKHAYK |
| 43611 | LAHWQGLTY |
| 43612 | LALCIRHSW |
| 43613 | LAPPHKLDL |
| 43614 | LASFTSPDP |
| 43615 | LASTYWYSQ |
| 43616 | LATLNAFQN |
| 43617 | LAVVRATLD |
| 43618 | MAASDWHVM |
| 43619 | MAAWLEWGL |
| 43620 | MAEIRTRHV |
| 43621 | MAHENTKAG |
| 43622 | MAMTLEISD |
| 43623 | MASTYIERE |
| 43624 | MAVPMKMHC |
| 43625 | MAYLVVRQH |
| 43626 | NAARMEQAT |
| 43627 | NACTTKVGS |
| 43628 | NADYCSFPI |
| 43629 | NAENHTKTC |
| 43630 | NAFSMDIHV |
| 43631 | NAQSDPHQG |
| 43632 | NASKYGDFL |
| 43633 | NASSTVINT |
| 43634 | NATANYCLG |
| 43635 | NATTHSHTS |
| 43636 | NAVDRGIAT |
| 43637 | NAVPIKYPY |
| 43638 | NAWSSNEEA |
| 43639 | PAQRVIANL |
| 43640 | QAALMTEQF |
| 43641 | QADRMLKTG |
| 43642 | QADRWTGII |
| 43643 | QAEWPGTMT |
| 43644 | QAGLGSDAP |
| 43645 | QAHCVNLSS |
| 43646 | QAMNSARTH |
| 43647 | QAMTMLMQH |
| 43648 | QANNYNRMD |
| 43649 | QANTGRVYS |
| 43650 | QAQCAMGAR |
| 43651 | QAQWMGCKW |
| 43652 | QARIENNQF |
| 43653 | QATLQPVCI |
| 43654 | QATQTYDWH |
| 43655 | QATYPITSE |
| 43656 | RADLWHIQN |
| 43657 | RAEDKWRSC |
| 43658 | RAERNSAEQ |
| 43659 | RAMAMDSSH |
| 43660 | RAMDDQHYG |
| 43661 | RAMLVEQWG |
| 43662 | RAQLFHQKT |
| 43663 | RAQPTSCCG |
| 43664 | RASVMPEAV |
| 43665 | SAAFQARLG |
| 43666 | SAAFSAFNV |
| 43667 | SAAWQLQMT |
| 43668 | SACQKEWCP |
| 43669 | SAEHKTHTM |
| 43670 | SAGRRNIGM |
| 43671 | SAHAGMHSN |
| 43672 | SAHKEDISG |
| 43673 | SAKSHLSNV |
| 43674 | SAKYFSFGV |
| 43675 | SANMFVFGK |
| 43676 | SANYYMTNM |
| 43677 | SARVCDCPW |
| 43678 | SASDPKNCY |
| 43679 | SASKFGWFS |
| 43680 | SASWLDDQS |
| 43681 | SATGMRDYA |
| 43682 | SAWFTIVSG |
| 43683 | TAAGYHFCK |
| 43684 | TAAPWRHQS |
| 43685 | TAAQFMDEL |
| 43686 | TACLCLERP |
| 43687 | TADPLCRGA |
| 43688 | TAGILDHYL |
| 43689 | TAHCVMNKQ |
| 43690 | TAHGIYTQF |
| 43691 | TAHPGWEGY |
| 43692 | TAKFFADWP |
| 43693 | TALIISQNL |
| 43694 | TALLLDANP |
| 43695 | TAMPITAIP |
| 43696 | TAMQSGKVS |
| 43697 | TANPEALVD |
| 43698 | TANYGAAFA |
| 43699 | TAQKSFMNC |
| 43700 | TATQCKAKE |
| 43701 | TAVTTWNMP |
| 43702 | VADSWAYNP |
| 43703 | VAEWIMEKC |
| 43704 | VAKQSADPI |
| 43705 | VALNVAQKG |
| 43706 | VAMKKMTDD |
| 43707 | VAMPTMKYK |
| 43708 | VAQLALDEK |
| 43709 | VARFTELCP |
| 43710 | VARTFGEEK |
| 43711 | VASKFAGSV |
| 43712 | VATAQWLSC |
| 43713 | VAVQFREWL |
| 43714 | WAEHRDMRN |
| 43715 | WALTGYNIT |
| 43716 | WAQVCHCGE |
| 43717 | YAAGAMEAS |
| 43718 | YADMPSERG |
| 43719 | YAELLQKMF |
| 43720 | YAGCMLAIQ |
| 43721 | YAGSHEENC |
| 43722 | YAHAIMGCQ |
| 43723 | YAHENQQTS |
| 43724 | YANMKMICE |
| 43725 | YAQTYYNKD |
| 43726 | YARRFWTCV |
| 43727 | YARTTLANK |
| 43728 | YASILTNQS |
| 43729 | YASQSWQQE |
| 43730 | YATELGEYG |
| 43731 | YAVFGHQSR |
| 43732 | YAVLQNHYG |
| 43733 | AALIGLIGD |
| 43734 | AANPTQWMT |
| 43735 | AATMQTVPT |
| 43736 | AAYMAKLRA |
| 43737 | CAEQIMRNQ |
| 43738 | CAQRSMRSM |
| 43739 | CAQSFTVKG |
| 43740 | CASHRKGAV |
| 43741 | DAENFVFKG |
| 43742 | DANMTRTRP |
| 43743 | DANVIAHNP |
| 43744 | EASFVVEPV |
| 43745 | FAIWEAHGR |
| 43746 | FASHNYAPV |
| 43747 | FASRVCVDG |
| 43748 | GAEQNNECT |
| 43749 | GAFTVEFSP |
| 43750 | HAMQEELPP |
| 43751 | HAQFRGSAT |
| 43752 | IACTQQISV |
| 43753 | IADWFDYEC |
| 43754 | IAMQYPEQY |
| 43755 | IAPSVNQAT |
| 43756 | IAPWWSTTT |
| 43757 | LAEAGQNQV |
| 43758 | LAEGLKRTG |
| 43759 | LAGKSGQQV |
| 43760 | LAIEMVDRS |
| 43761 | LAISFKGKW |
| 43762 | LARGKDVAE |
| 43763 | MAKIGYYMI |
| 43764 | MAKPNPPLG |
| 43765 | NAAKYFWKQ |
| 43766 | NADLQIFFR |
| 43767 | NALPESMTC |
| 43768 | NAYPFVDHR |
| 43769 | PAAMDYLMD |
| 43770 | PACQETGMF |
| 43771 | PAEAEKIGR |
| 43772 | QAEFRCGNH |
| 43773 | QAFCEPNTP |
| 43774 | QAGKWAVCE |
| 43775 | QALWYEDMT |
| 43776 | QASCTHVWD |
| 43777 | RAEWKQWSC |
| 43778 | SAEWMQSWH |
| 43779 | SAEWTHNNQ |
| 43780 | SAGMYARVA |
| 43781 | SATMCDSQS |
| 43782 | SAYWFQMAG |
| 43783 | TAEHIKVDL |
| 43784 | TAISEIPAP |
| 43785 | TAKMVAAME |
| 43786 | TALMLNMAS |
| 43787 | TAVTTNKWA |
| 43788 | VAAKNYKER |
| 43789 | VAMMVAGFQ |
| 43790 | VAMTDAKMC |
| 43791 | VANVAIADM |
| 43792 | VASGSTEVC |
| 43793 | VATMIATQL |
| 43794 | WAHKDWSNW |
| 43795 | WAMEDGKSA |
| 43796 | YAALTYYYS |
| 43797 | ICKQNYSRT |
| 43798 | IFKEGIHYQ |
| 43799 | IPKNMGGCE |
| 43800 | IRKEVAVVP |
| 43801 | IRKNYRMKQ |
| 43802 | ITKNRDRWP |
| 43803 | IVKLVLPVG |
| 43804 | IVKQEHESY |
| 43805 | IYKPNTQLQ |
| 43806 | IYKTRHGWW |
| 43807 | IYKVLKPVH |
| 43808 | LCKQLGKQE |
| 43809 | LGKQEAVTN |
| 43810 | LGKWKIQGT |
| 43811 | LIKPLEISD |
| 43812 | LMKCLNWCC |
| 43813 | LMKCNEAFT |
| 43814 | LQKSWNMLQ |
| 43815 | LVKRKELET |
| 43816 | LVKWTNVLP |
| 43817 | LYKMMYAFR |
| 43818 | MCKLKLEIP |
| 43819 | MCKNQNKPA |
| 43820 | MHKPEGMNG |
| 43821 | MIKPSVEGM |
| 43822 | MLKCTHDLP |
| 43823 | MNKTMINYA |
| 43824 | MQKLMALQA |
| 43825 | MQKTIQQWM |
| 43826 | MWKVYEKSE |
| 43827 | NHKWICLTR |
| 43828 | NIKSRMIEF |
| 43829 | NMKSQWQPS |
| 43830 | NNKFWDYDP |
| 43831 | NQKATTASY |
| 43832 | NQKWSTWNN |
| 43833 | NVKCMQHMV |
| 43834 | PRKNQVDLG |
| 43835 | QCKQPLVIN |
| 43836 | QDKTKSCMY |
| 43837 | QGKYSANWY |
| 43838 | QKKFIQCYG |
| 43839 | QLKAPPAAH |
| 43840 | QMKMLWFNV |
| 43841 | QNKPASMHP |
| 43842 | QQKPQECYS |
| 43843 | QVKDVVGNV |
| 43844 | RGKTYEHGN |
| 43845 | RPKTFAALR |
| 43846 | RSKQARISH |
| 43847 | SCKLNLHEG |
| 43848 | SFKFYAEEA |
| 43849 | SGKQGNWRL |
| 43850 | SIKELVGCG |
| 43851 | SIKMIDCFG |
| 43852 | SMKWFITDD |
| 43853 | SVKYCQSKD |
| 43854 | TFKSYEMET |
| 43855 | TFKYHYNAC |
| 43856 | THKWGWWIA |
| 43857 | TIKPKNEWC |
| 43858 | TIKPYYPYQ |
| 43859 | TKKKRNGHL |
| 43860 | TRKDTLVAS |
| 43861 | TSKTCFAEK |
| 43862 | TTKEDFVCS |
| 43863 | TTKYTQSIF |
| 43864 | TVKPAVPGE |
| 43865 | VEKSSIMNR |
| 43866 | VFKLPEPVV |
| 43867 | VSKCGVSHI |
| 43868 | VTKYKYEGM |
| 43869 | WKKMKYTWK |
| 43870 | WMKPEFHYD |
| 43871 | YSKYKRLWI |
| 43872 | AEKPWNAMN |
| 43873 | AIKFKYAPD |
| 43874 | ANKVGTAGN |
| 43875 | AQKEYSKYL |
| 43876 | CKKMGLHVM |
| 43877 | CSKFRECCG |
| 43878 | DFKTMNYMV |
| 43879 | DFKWIQDQG |
| 43880 | DHKILGAIP |
| 43881 | ELKVAISSG |
| 43882 | ENKTQFAYL |
| 43883 | FRKMGCGKT |
| 43884 | GLKLNLFQA |
| 43885 | GSKTCQQVM |
| 43886 | LNKGQPLFM |
| 43887 | LTKETGQVV |
| 43888 | MLKTARWRD |
| 43889 | MSKPRVCCA |
| 43890 | MVKTGVEEM |
| 43891 | MWKFAQLRL |
| 43892 | MYKPEQWCM |
| 43893 | QIKSCKNTD |
| 43894 | STKQHPHLN |
| 43895 | STKVPWWFP |
| 43896 | TMKFSHESA |
| 43897 | TYKYKESSE |
| 43898 | VQKSWEYDN |
| 43899 | WSKKDIWAD |
| 43900 | IIPKDNKWR |
| 43901 | IPAKFRSWW |
| 43902 | ISVKGLTTH |
| 43903 | IYCKIKEWG |
| 43904 | LDHKCAAGM |
| 43905 | LHLKLINHC |
| 43906 | LLEKKQASG |
| 43907 | LSQKPSNLI |
| 43908 | LYAKKMAGY |
| 43909 | LYAKKMVGY |
| 43910 | MGSKIHFMA |
| 43911 | MQGKNMAVT |
| 43912 | NCVKAMDWY |
| 43913 | NHEKTIASF |
| 43914 | NIDKRGPMM |
| 43915 | NMLKHMFGQ |
| 43916 | NMVKYIHMG |
| 43917 | NRAKEHWHQ |
| 43918 | NSYKLDWGW |
| 43919 | NTGKMEEKR |
| 43920 | PFEKWNSQS |
| 43921 | PFRKSAGYM |
| 43922 | QHTKPNCRD |
| 43923 | QLCKVGRDN |
| 43924 | QTEKLMRLD |
| 43925 | QTNKWDVVT |
| 43926 | QTWKLITHG |
| 43927 | QVHKGQKFS |
| 43928 | QVNKITWVN |
| 43929 | QVTKFVTEG |
| 43930 | QWCKLINMC |
| 43931 | QWVKYHDIW |
| 43932 | QYTKKYWNH |
| 43933 | RVHKGQKFS |
| 43934 | SCAKFDVVE |
| 43935 | SDSKLHQVA |
| 43936 | SGTKYCCWW |
| 43937 | SIGKWPAYE |
| 43938 | SMMKCPTQY |
| 43939 | SVWKCERIS |
| 43940 | TCTKAVDHC |
| 43941 | TYEKFNKHQ |
| 43942 | TYIKSYQMA |
| 43943 | VRDKGVQCA |
| 43944 | VSQKSPTAW |
| 43945 | WCTKCNYNQ |
| 43946 | WCVKRMENH |
| 43947 | WVEKHLMLG |
| 43948 | YSQKPEEAN |
| 43949 | CTYKTGSPA |
| 43950 | CVLKWATMW |
| 43951 | DQMKWSQMG |
| 43952 | DWVKATQMC |
| 43953 | ESDKEERMT |
| 43954 | EVVKMPNHR |
| 43955 | EYIKWDGTP |
| 43956 | FCQKIEYYH |
| 43957 | FGPKLTWHT |
| 43958 | GCGKCECWN |
| 43959 | GTFKNPVHQ |
| 43960 | GVTKLEVWP |
| 43961 | GWDKKRDFR |
| 43962 | IPIKLPPQG |
| 43963 | LEAKVAQYY |
| 43964 | MDCKYPRDQ |
| 43965 | NDGKIQFSG |
| 43966 | NLSKLAQMP |
| 43967 | NLSKLTQMP |
| 43968 | NYAKLFQDI |
| 43969 | QKRKLQVYS |
| 43970 | QPAKKFWDF |
| 43971 | QSSKWMWPL |
| 43972 | QWYKMGYNR |
| 43973 | RGPKDTNVK |
| 43974 | RSLKYEGQQ |
| 43975 | SPNKSPFPG |
| 43976 | SSIKPMLSP |
| 43977 | SYHKQERCQ |
| 43978 | TCVKAGLCL |
| 43979 | TGLKVIGQH |
| 43980 | TIMKPLCFA |
| 43981 | TNHKFRCDR |
| 43982 | TSSKCTMDA |
| 43983 | VHPKVQRVT |
| 43984 | YSDKMESSN |
| 43985 | IHCMFGAGA |
| 43986 | IQQYFNWCE |
| 43987 | IRDMFEVGR |
| 43988 | LEFEFYWDE |
| 43989 | LIHPFARGN |
| 43990 | LIQAFPRDT |
| 43991 | LPTPFMAHL |
| 43992 | LSRDFCNWF |
| 43993 | LVTSFASML |
| 43994 | MCDHFQMAY |
| 43995 | MPRCFDAAA |
| 43996 | MPVMFGLHC |
| 43997 | NMYPFMKYQ |
| 43998 | NWITFSGAV |
| 43999 | QCDLFSEGY |
| 44000 | QHSVFMQIP |
| 44001 | QNIRFTTDM |
| 44002 | QSNHFEQEK |
| 44003 | QYGSFNQNV |
| 44004 | REEIFSYMV |
| 44005 | RGIVFSQDN |
| 44006 | RKADFMHIT |
| 44007 | RPIWFIFVL |
| 44008 | RQQEFDRCR |
| 44009 | RTDHFGHWE |
| 44010 | RVEMFKWRT |
| 44011 | SCHGFQNWP |
| 44012 | SIGLFMKDS |
| 44013 | SKEWFSGTS |
| 44014 | SNAPFEQCN |
| 44015 | SNLSFGLTA |
| 44016 | SQGYFDANL |
| 44017 | STQMFANVQ |
| 44018 | TCMYFSCAL |
| 44019 | TEAWFQSAF |
| 44020 | TIRNFWLQN |
| 44021 | TNMLFLVWP |
| 44022 | TPCRFNSQR |
| 44023 | TQSCFHLEN |
| 44024 | TRQFFHHGD |
| 44025 | TVEQFTNSV |
| 44026 | TVRMFCRAI |
| 44027 | TWHSFPNQE |
| 44028 | TYISFQDEK |
| 44029 | VSQSFDHVN |
| 44030 | VWQPFSGDH |
| 44031 | YCCWFEHRL |
| 44032 | YGCHFQRQE |
| 44033 | YHMGFTGMG |
| 44034 | YMSSFTCHW |
| 44035 | YPMTFRAAC |
| 44036 | YYNNFICDK |
| 44037 | ACVAFSQQM |
| 44038 | AHEIFNLQV |
| 44039 | AHTQFLSAD |
| 44040 | AMQSFDMPG |
| 44041 | ARFLFYFDV |
| 44042 | CCVYFNRQM |
| 44043 | CNIDFTADY |
| 44044 | CSQIFWERG |
| 44045 | CTPQFNRGF |
| 44046 | DEIAFWDGE |
| 44047 | DGLMFHQQR |
| 44048 | DIDQFASVN |
| 44049 | DREAFDMAS |
| 44050 | DSQWFEHNA |
| 44051 | DTGPFMLFQ |
| 44052 | ETRPFEQVW |
| 44053 | FTNRFQLMA |
| 44054 | GCCHFCDHL |
| 44055 | HRMYFLRWP |
| 44056 | IICGFFAWS |
| 44057 | LGHQFNNWT |
| 44058 | MDGYFGFAV |
| 44059 | MGSNFVKQF |
| 44060 | NICGFFAWS |
| 44061 | QHCQFEQAI |
| 44062 | QKDIFPMQM |
| 44063 | QMELFCQQD |
| 44064 | QNTEFMDHY |
| 44065 | QTSDFAMSS |
| 44066 | QVMPFVIEG |
| 44067 | SKMNFGTAN |
| 44068 | SKTEFSLKM |
| 44069 | TGDLFCCHL |
| 44070 | TQGLFAQVG |
| 44071 | VCFHFRDAA |
| 44072 | VCMLFQHEA |
| 44073 | VHSRFGQYT |
| 44074 | WQPRFYHIW |
| 44075 | YEDPFRHYS |
| 44076 | ICHSGNRLL |
| 44077 | ICQGSNSMA |
| 44078 | IGTVRNWCW |
| 44079 | IICPTNEYM |
| 44080 | IKILLNEMT |
| 44081 | IQCFHNMEE |
| 44082 | IVMCHNVYP |
| 44083 | IVNLNNHHW |
| 44084 | LCAQHNIVC |
| 44085 | LETEGNPFY |
| 44086 | LGIGNNWEQ |
| 44087 | LITPPNWWH |
| 44088 | LLEIMNEQC |
| 44089 | LLEWYNHMS |
| 44090 | LMILRNTHQ |
| 44091 | LQMPYNWQT |
| 44092 | LRHICNCGQ |
| 44093 | LSLSKNCAG |
| 44094 | LTPLANQYC |
| 44095 | LTRFNNDLP |
| 44096 | LVPRINMLW |
| 44097 | LYELHNTWD |
| 44098 | MFIQDNAPT |
| 44099 | MLLYPNNTN |
| 44100 | MMCSNNSHR |
| 44101 | MQSPENTVY |
| 44102 | MTCLQNQSE |
| 44103 | MTIANNIRN |
| 44104 | MTYNYNSKD |
| 44105 | MVWICNAPR |
| 44106 | NCGYKNNKA |
| 44107 | NICFVNDAL |
| 44108 | NILSPNCYE |
| 44109 | NKVGMNYMQ |
| 44110 | NMCHHNWRD |
| 44111 | NPAYKNWGW |
| 44112 | NQIYTNPWG |
| 44113 | NSLISNAWT |
| 44114 | NTMTPNFRK |
| 44115 | NYVAKNYMV |
| 44116 | PKFIANRQE |
| 44117 | PNYWINYQN |
| 44118 | PRCYINWRY |
| 44119 | PWQFTNLEK |
| 44120 | QCCHPNGYQ |
| 44121 | QNCWYNFFR |
| 44122 | QQCNTNTDS |
| 44123 | QVVTVNASD |
| 44124 | RIGIANDWV |
| 44125 | SDGTINRMN |
| 44126 | SHLQCNYMR |
| 44127 | SLTVWNQNG |
| 44128 | STMWRNRES |
| 44129 | STVYWNMET |
| 44130 | SYTSGNESW |
| 44131 | TGAPGNWFH |
| 44132 | TRNAYNCHQ |
| 44133 | TSSWVNWDP |
| 44134 | TTMPENIPL |
| 44135 | TTRFHNNVM |
| 44136 | TVWNTNTGW |
| 44137 | VCRNGNRQA |
| 44138 | VIFPENSHY |
| 44139 | VLTWDNPDE |
| 44140 | VTHATNVLE |
| 44141 | WNGFMNQGY |
| 44142 | WPVRLNEYK |
| 44143 | YIAVPNKMS |
| 44144 | YYECRNYSD |
| 44145 | AKVYSNLYG |
| 44146 | ATILVNRDG |
| 44147 | AWFTHNALP |
| 44148 | CIHHRNDKV |
| 44149 | CNYDTNLRP |
| 44150 | CTQSINGLI |
| 44151 | CTVLINCRD |
| 44152 | CVTWENVMT |
| 44153 | DLVHCNNHM |
| 44154 | DRGYYNSMM |
| 44155 | DTIHENEGV |
| 44156 | EPHIMNSHE |
| 44157 | EQRVYNHQY |
| 44158 | FTVTMNCTA |
| 44159 | GGALDNGQC |
| 44160 | GPDRYNEES |
| 44161 | GSLALNVRQ |
| 44162 | GTQRDNTGF |
| 44163 | HCICNNSGA |
| 44164 | HMTLGNLCP |
| 44165 | ICGWENCSE |
| 44166 | LCINVNPSI |
| 44167 | LFTEINGCY |
| 44168 | LMCQMNNRS |
| 44169 | MPGFENKMP |
| 44170 | MPRDENHIG |
| 44171 | NFDCSNDQT |
| 44172 | NLLVSNVTT |
| 44173 | PDGPMNLLG |
| 44174 | PMMMANRIA |
| 44175 | QHRQANGIH |
| 44176 | QMSYVNQYR |
| 44177 | QSVSNNRRA |
| 44178 | QTFMTNFDT |
| 44179 | QYSPANQWD |
| 44180 | RTFLTNPKD |
| 44181 | SMHDYNFNV |
| 44182 | STELKNMER |
| 44183 | SVLYSNYFA |
| 44184 | TGAMTNYCY |
| 44185 | TMMQNNTEV |
| 44186 | TRDTHNSVD |
| 44187 | TRIHVNRYG |
| 44188 | TTWLDNEDA |
| 44189 | VIRPINQEW |
| 44190 | WQAPHNQSY |
| 44191 | YVITANNHQ |
| 44192 | ICQHIGSWG |
| 44193 | IDHDYASDS |
| 44194 | ILIMILSHS |
| 44195 | IPRFVYSPV |
| 44196 | ITVADRSSH |
| 44197 | LEGATESLW |
| 44198 | LLPLHGSWH |
| 44199 | MCLAPTSMH |
| 44200 | MCSPEPSLQ |
| 44201 | MEHQQDSRF |
| 44202 | MGDDSMSEH |
| 44203 | MPRTCMSTN |
| 44204 | NCEQWKSMF |
| 44205 | NDQTSSSAW |
| 44206 | NIQFHTSGQ |
| 44207 | NKDVDRSHV |
| 44208 | NLVRWESGW |
| 44209 | NMSPWMSDR |
| 44210 | NPCIVISSV |
| 44211 | NSYHLESMD |
| 44212 | NVAMYASSE |
| 44213 | NVLCVKSVQ |
| 44214 | NVMICHSPA |
| 44215 | NYCTLMSAC |
| 44216 | PILITHSVE |
| 44217 | QIGWEASSL |
| 44218 | QIVELHSLG |
| 44219 | QIVQAMSSE |
| 44220 | QLADNHSRP |
| 44221 | QSLYDRSGT |
| 44222 | QSTCYFSNC |
| 44223 | QTREGVSYY |
| 44224 | QVDMARSSF |
| 44225 | QVMENSSWA |
| 44226 | RECVHLSCP |
| 44227 | REGLLWSNS |
| 44228 | RISQQWSRS |
| 44229 | RTIQKESHV |
| 44230 | RWCMKISAN |
| 44231 | SCTHVMSNA |
| 44232 | SHELQLSGL |
| 44233 | SHMNQASMP |
| 44234 | SITRWVSAE |
| 44235 | SLYMEMSWQ |
| 44236 | SWCSIHSPN |
| 44237 | SYIEWGSWM |
| 44238 | TCERKRSDK |
| 44239 | TCTYDKSQI |
| 44240 | THIQCRSMM |
| 44241 | THWWQPSSG |
| 44242 | TKHMIQSYG |
| 44243 | TSARCISAE |
| 44244 | TSTHVKSYQ |
| 44245 | TTHFAVSGI |
| 44246 | TTSHCVSPS |
| 44247 | TVHMCQSFV |
| 44248 | VGTAAHSQR |
| 44249 | VIAQELSAK |
| 44250 | VSDWGHSDS |
| 44251 | VWWREDSTN |
| 44252 | WLEPCVSSL |
| 44253 | WPETVGSSE |
| 44254 | WYAPAESSN |
| 44255 | YCCRPVSDF |
| 44256 | YHMDGDSRH |
| 44257 | ADETQYSCH |
| 44258 | AFNQHTSMD |
| 44259 | AISPWMSCA |
| 44260 | AMYNVSSAA |
| 44261 | AMYQNPSPC |
| 44262 | ARAEWKSKD |
| 44263 | CVCADTSVF |
| 44264 | DIELVTSEQ |
| 44265 | ELAAPHSQV |
| 44266 | ERATLKSMC |
| 44267 | ERMWKTSDN |
| 44268 | ESIRLRSSQ |
| 44269 | FNAVHMSLE |
| 44270 | GGRFNQSWA |
| 44271 | GHFMTLSKG |
| 44272 | GNERMFSSA |
| 44273 | HLTMWESFD |
| 44274 | LCTFNGSNG |
| 44275 | LKGTMMSPI |
| 44276 | LTYLKRSIG |
| 44277 | MLNLHGSDM |
| 44278 | MSIPASSEV |
| 44279 | NIGYRKSSG |
| 44280 | NRESDVSTG |
| 44281 | PCQWPVSRA |
| 44282 | PCVYECSLH |
| 44283 | PTIWLHSYL |
| 44284 | PVCPWGSFD |
| 44285 | PWVAVESAE |
| 44286 | QQTRHRSCV |
| 44287 | RTEWHQSQA |
| 44288 | SMQPTGSGW |
| 44289 | STQWEQSMQ |
| 44290 | SWVSKFSEI |
| 44291 | TNMPMPSYE |
| 44292 | TQGRMYSCW |
| 44293 | TWGFNRSYC |
| 44294 | VQHCSHSLD |
| 44295 | VTYEAKSQW |
| 44296 | WDSQPQSGV |
| 44297 | WELNNPSVM |
| 44298 | WLTWHFSSF |
| 44299 | IIATGWECD |
| 44300 | ISLPMLECI |
| 44301 | LKIYTKCCD |
| 44302 | LSSHSEICQ |
| 44303 | MCFDIATCA |
| 44304 | MEHFLQVCN |
| 44305 | MKWPAFACA |
| 44306 | MMNVHQNCE |
| 44307 | MPVRLTKCS |
| 44308 | MWVDCCECH |
| 44309 | NGGQKATCP |
| 44310 | NGWFVRICG |
| 44311 | NNASPSYCG |
| 44312 | NRPHQWLCC |
| 44313 | NSHACSECS |
| 44314 | PNNQCSYCC |
| 44315 | PRTEDLQCI |
| 44316 | QCMAHIQCD |
| 44317 | QEMDGWYCC |
| 44318 | QGAIYEVCL |
| 44319 | QIFLWFYCG |
| 44320 | QKQSTVICE |
| 44321 | QLNVTPICC |
| 44322 | QMAPYSICQ |
| 44323 | QNGNIEKCQ |
| 44324 | QQNPNGCCH |
| 44325 | QVSSHVECV |
| 44326 | RLSAGDACY |
| 44327 | RPVLECNCE |
| 44328 | RQYIQDVCD |
| 44329 | RREPTKCCW |
| 44330 | RVTEWFQCG |
| 44331 | RWCGKGYCV |
| 44332 | SFSYAECCA |
| 44333 | SHENQWACI |
| 44334 | SHMFQHTCG |
| 44335 | SNTLYDTCY |
| 44336 | SQFMDDDCA |
| 44337 | SRNAMQYCP |
| 44338 | SSHIDGICN |
| 44339 | TELVYMTCQ |
| 44340 | TGDSRMDCQ |
| 44341 | TKGFMSVCR |
| 44342 | TNYPNWICH |
| 44343 | TQEAISDCF |
| 44344 | TQQFCHDCQ |
| 44345 | TTGMLENCN |
| 44346 | TWTQMIDCY |
| 44347 | TYVRRWMCL |
| 44348 | VIHGSLQCQ |
| 44349 | VIMMDGYCT |
| 44350 | VIMTQMNCW |
| 44351 | VPNTKDACF |
| 44352 | VWCGNQWCN |
| 44353 | WFTNVLWCG |
| 44354 | YPIMERWCH |
| 44355 | YSEICDRCI |
| 44356 | AFAQLGQCH |
| 44357 | ALVLRCICL |
| 44358 | CHPDKMECP |
| 44359 | CVMVIWLCS |
| 44360 | DGSCKWNCH |
| 44361 | DSMRKYQCR |
| 44362 | DTVMQAACW |
| 44363 | GGPHREFCI |
| 44364 | HTIDKMNCP |
| 44365 | IPYQQDCCA |
| 44366 | LILHMLECQ |
| 44367 | MHTSMLICC |
| 44368 | MKAATMVCL |
| 44369 | MKFTLPQCN |
| 44370 | NCHRNDACD |
| 44371 | NENQMMHCA |
| 44372 | NTAQQLCCE |
| 44373 | NTTQRALCG |
| 44374 | PTRTRRTCR |
| 44375 | QINLDSQCM |
| 44376 | QSAIEEVCR |
| 44377 | QSHPRITCA |
| 44378 | QVQMGRDCG |
| 44379 | RCITPTGCI |
| 44380 | SCCYCPTCC |
| 44381 | SMCFKGTCE |
| 44382 | SRNANDGCF |
| 44383 | SVFMRDECC |
| 44384 | TCWQWLKCE |
| 44385 | TMDRQPMCI |
| 44386 | TSSTEQMCV |
| 44387 | IGRITMGDR |
| 44388 | IHTLKHDYR |
| 44389 | IMFMVAFDR |
| 44390 | LHVPPSWAR |
| 44391 | LMCTMLTDR |
| 44392 | LNMELMHLR |
| 44393 | MDFLSCGLR |
| 44394 | MELDVSNDR |
| 44395 | MFMMMHDIR |
| 44396 | MNHWNMHMR |
| 44397 | NDPHQHDAR |
| 44398 | NFLDCSERR |
| 44399 | NPNLIEKHR |
| 44400 | QCLLTLQNR |
| 44401 | QEHHVFGVR |
| 44402 | QITHLQVGR |
| 44403 | QSSFNPDMR |
| 44404 | QYEVKMWSR |
| 44405 | RNDDNLERR |
| 44406 | RTFYKQVAR |
| 44407 | SCSQIQEIR |
| 44408 | SEGCRMPPR |
| 44409 | SFAVMMEDR |
| 44410 | SPNLIEKHR |
| 44411 | SSSSRRDYR |
| 44412 | SVYLKLRIR |
| 44413 | SYLQWHVTR |
| 44414 | TGYFYMAHR |
| 44415 | TPAFTHWER |
| 44416 | TPMSTQNVR |
| 44417 | TTPQYMRER |
| 44418 | VNMPLYKFR |
| 44419 | WEHHCAEWR |
| 44420 | WLPFAMTNR |
| 44421 | YCYRAKEQR |
| 44422 | YIEPPALGR |
| 44423 | YINMNAQER |
| 44424 | YKLCRVKGR |
| 44425 | YMLVDMIFR |
| 44426 | DHAHYWYGR |
| 44427 | DSVSQQVER |
| 44428 | ECCNYVWER |
| 44429 | ESQYSQGTR |
| 44430 | FMSMQGDQR |
| 44431 | GKNCWPIKR |
| 44432 | HTGPMLGDR |
| 44433 | HWCTIGKLR |
| 44434 | MGADYGVWR |
| 44435 | NFNQCTIYR |
| 44436 | QKQGTICER |
| 44437 | QSAIEEVYR |

### Example 5

AAV5 Variants with Tissue Tropism in CNS

This example describes engineered AAV5 variants with tissue tropism in CNS that were discovered using the methods and systems described in EXAMPLE 1-EXAMPLE 2.

TABLE 8 is a list of 1000 variants (CNS variants) that had the highest normalized UMI counts across two cortical sequencing samples and absent in two liver sequencing samples. Minimal inclusion criteria for this list included presence in both quality filtered cortex NGS data sets; and exclusion criteria were detection of a given variant in either of two independent liver sequencing samples. The total number of variants that meet these criteria following analysis of the four data sets is 2108. UMI counts were then normalized independently for the two separate cortex NGS data sets, and the variant sequences were then rank ordered by the sum of the two independently normalized UMI counts (maximum possible=200%), and the top 1000 are listed here (SEQ ID NO: 115-SEQ ID NO: 1114). Variant residues are at positions 581-589.

TABLE 8

| | | norm 2 |
|---|---|---|
| | | samples |
| | | (max |
| | | possible |
| SEQ ID NO | var_aa | score 200%) |
| SEQ ID NO: 115 | RVTIMFTGT | 112.28% |
| SEQ ID NO: 116 | YTGCIDGFL | 100.01% |
| SEQ ID NO: 117 | TNMSTQPCW | 97.08% |
| SEQ ID NO: 118 | VWTTVCNDY | 96.78% |
| SEQ ID NO: 119 | DYANTLKQS | 91.84% |
| SEQ ID NO: 120 | EFVLGCQGI | 76.88% |
| SEQ ID NO: 121 | TIMWTMMRE | 76.32% |
| SEQ ID NO: 122 | SPMVVTHSD | 76.07% |
| SEQ ID NO: 123 | DWYDIWTLV | 69.59% |
| SEQ ID NO: 124 | REFAISGGN | 69.44% |
| SEQ ID NO: 125 | SITNCNHDR | 69.01% |
| SEQ ID NO: 126 | HIQEELEEQ | 64.92% |
| SEQ ID NO: 127 | DINGKKNIC | 64.05% |
| SEQ ID NO: 128 | TLAEHFTSH | 63.45% |
| SEQ ID NO: 129 | QCQSQEWFH | 60.94% |
| SEQ ID NO: 130 | ARSGINGHE | 60.24% |
| SEQ ID NO: 131 | CIRQYDAID | 59.65% |
| SEQ ID NO: 132 | ILIFESRIG | 58.77% |
| SEQ ID NO: 133 | HFADNQVVQ | 56.73% |
| SEQ ID NO: 134 | NESHVERHC | 56.14% |
| SEQ ID NO: 135 | TMVTSVTLP | 55.86% |
| SEQ ID NO: 136 | PTLSCPYHK | 54.39% |
| SEQ ID NO: 137 | TVAKVLSGH | 53.82% |
| SEQ ID NO: 138 | KIFITTDEG | 52.65% |
| SEQ ID NO: 139 | KSAQVYWHP | 52.63% |
| SEQ ID NO: 140 | CSALFAQHD | 52.05% |
| SEQ ID NO: 141 | AGSACMPAH | 50.59% |
| SEQ ID NO: 142 | IVIWQGEWI | 50.29% |
| SEQ ID NO: 143 | SYCEVEDAP | 50.00% |
| SEQ ID NO: 144 | DCSWWLQLG | 49.42% |
| SEQ ID NO: 145 | NFCKQGNTC | 47.08% |
| SEQ ID NO: 146 | HIHDTMSDY | 46.81% |
| SEQ ID NO: 147 | QYVHMLGEC | 46.50% |
| SEQ ID NO: 148 | RQHHCLPVN | 46.22% |
| SEQ ID NO: 149 | AHVDHQCSG | 45.43% |
| SEQ ID NO: 150 | SFSDTHLGW | 45.40% |
| SEQ ID NO: 151 | AASQMWQRG | 45.04% |
| SEQ ID NO: 152 | NDCTCRAIM | 44.16% |
| SEQ ID NO: 153 | NSHEYLDRH | 43.86% |
| SEQ ID NO: 154 | DWIWGTDMW | 43.60% |
| SEQ ID NO: 155 | MIFYRTYGF | 42.40% |
| SEQ ID NO: 156 | HPAPLNMPH | 42.11% |
| SEQ ID NO: 157 | SQQHCQHDQ | 41.58% |
| SEQ ID NO: 158 | HEVMMCPAP | 41.01% |
| SEQ ID NO: 159 | KRTDLHAAD | 40.95% |
| SEQ ID NO: 160 | EYFKPKSQD | 40.94% |
| SEQ ID NO: 161 | KLAHSWCIW | 40.94% |
| SEQ ID NO: 162 | NAMANFSVR | 40.64% |
| SEQ ID NO: 163 | EVTTPRPPT | 39.50% |
| SEQ ID NO: 164 | YFHDEQIDY | 38.90% |
| SEQ ID NO: 165 | LTHQGGRQR | 38.31% |
| SEQ ID NO: 166 | AQWYMWCWD | 37.73% |
| SEQ ID NO: 167 | RMMWQSHCD | 37.44% |
| SEQ ID NO: 168 | EIHKRIAMT | 36.55% |
| SEQ ID NO: 169 | CMSCCKWPH | 36.42% |
| SEQ ID NO: 170 | DFQRPTETQ | 35.67% |
| SEQ ID NO: 171 | SFMPCHCRG | 35.67% |
| SEQ ID NO: 172 | LTHNTPFPK | 35.40% |
| SEQ ID NO: 173 | HYVVRHEDP | 35.14% |
| SEQ ID NO: 174 | FQEAPPHVH | 34.80% |
| SEQ ID NO: 175 | YAELEWKVC | 33.92% |
| SEQ ID NO: 176 | RCVAEPNEP | 33.37% |
| SEQ ID NO: 177 | GLIFHQSLS | 33.34% |
| SEQ ID NO: 178 | ASHGMDEYN | 33.33% |
| SEQ ID NO: 179 | PIACRAINW | 33.33% |
| SEQ ID NO: 180 | NCKPDQIAM | 32.69% |
| SEQ ID NO: 181 | FRMWFTECI | 31.87% |
| SEQ ID NO: 182 | MGYAKHDSH | 31.59% |
| SEQ ID NO: 183 | DTSCLELMQ | 31.29% |
| SEQ ID NO: 184 | KVAGGHETT | 31.16% |
| SEQ ID NO: 185 | YSRYHHSLL | 30.99% |
| SEQ ID NO: 186 | VADTWQVHC | 30.43% |
| SEQ ID NO: 187 | LWGYGAVNM | 29.25% |
| SEQ ID NO: 188 | FGRQAYVAW | 29.24% |
| SEQ ID NO: 189 | EEVQFTQQD | 29.24% |
| SEQ ID NO: 190 | GTGHIGNAT | 29.25% |
| SEQ ID NO: 191 | AYTYNSKLY | 28.95% |
| SEQ ID NO: 192 | FIFRKCDHW | 28.37% |
| SEQ ID NO: 193 | QCVDCPPYC | 27.79% |
| SEQ ID NO: 194 | ETPPLCHGP | 27.78% |
| SEQ ID NO: 195 | RMTPNNCQR | 27.28% |
| SEQ ID NO: 196 | NSTPMACME | 26.91% |
| SEQ ID NO: 197 | STHPLNLYH | 26.90% |
| SEQ ID NO: 198 | TTVPDPGPR | 26.32% |
| SEQ ID NO: 199 | LIWSVYQDE | 26.09% |
| SEQ ID NO: 200 | YDNHQKARN | 26.02% |
| SEQ ID NO: 201 | SIYQKWVTN | 25.92% |
| SEQ ID NO: 202 | MQSMQHWHP | 25.74% |
| SEQ ID NO: 203 | TLVSCDQGN | 25.73% |
| SEQ ID NO: 204 | WSITGQPWF | 25.73% |
| SEQ ID NO: 205 | YQKVEYWNY | 25.73% |
| SEQ ID NO: 206 | AHYQQSTDR | 25.44% |
| SEQ ID NO: 207 | QVRVITHKS | 25.17% |
| SEQ ID NO: 208 | WAAKPGDPK | 25.15% |
| SEQ ID NO: 209 | RSDNDWCFL | 25.15% |
| SEQ ID NO: 210 | ETHRGTVGL | 24.89% |
| SEQ ID NO: 211 | KCSVQEKKA | 24.89% |
| SEQ ID NO: 212 | WVEPEQCFG | 24.56% |
| SEQ ID NO: 213 | NHQQCPKCP | 24.28% |
| SEQ ID NO: 214 | AAHSAPFFD | 24.27% |
| SEQ ID NO: 215 | KGQRRDSVS | 24.07% |
| SEQ ID NO: 216 | DVMQNQCKS | 23.98% |
| SEQ ID NO: 217 | ALVYSGWIC | 23.72% |
| SEQ ID NO: 218 | FAKARHNWR | 23.69% |
| SEQ ID NO: 219 | TYIQYPTNA | 23.69% |
| SEQ ID NO: 220 | VSKFIAASW | 23.69% |
| SEQ ID NO: 221 | RQREGKHGN | 23.68% |
| SEQ ID NO: 222 | MAAHTKYRF | 23.40% |
| SEQ ID NO: 223 | FVGLMNQCG | 23.39% |
| SEQ ID NO: 224 | TWQSMVNSE | 23.15% |
| SEQ ID NO: 225 | SAITCRMLK | 23.10% |
| SEQ ID NO: 226 | SRHYEQFEC | 23.10% |
| SEQ ID NO: 227 | VTAGQLGLN | 23.10% |
| SEQ ID NO: 228 | DSQYHVKCG | 22.82% |
| SEQ ID NO: 229 | LSECSDRAN | 22.51% |
| SEQ ID NO: 230 | YAYPTEGQI | 22.22% |
| SEQ ID NO: 231 | KRFVDMPEM | 21.95% |
| SEQ ID NO: 232 | VHKFQGNFG | 21.94% |
| SEQ ID NO: 233 | RGHEPPMWL | 21.93% |
| SEQ ID NO: 234 | QKCYCDEQC | 21.64% |
| SEQ ID NO: 235 | RHAPTDISP | 21.60% |
| SEQ ID NO: 236 | MTGIGKCWN | 21.05% |
| SEQ ID NO: 237 | QAYCCMHQY | 20.48% |
| SEQ ID NO: 238 | KHDDCCTGT | 20.47% |
| SEQ ID NO: 239 | KWYQFGMVA | 20.42% |
| SEQ ID NO: 240 | LTWECKFGG | 20.18% |
| SEQ ID NO: 241 | AIVQQHYYP | 19.88% |
| SEQ ID NO: 242 | EENVWMQYQ | 19.88% |
| SEQ ID NO: 243 | NSQPMDFLP | 19.88% |
| SEQ ID NO: 244 | FDTAFQGDI | 19.59% |
| SEQ ID NO: 245 | QMEARMDCE | 19.49% |
| SEQ ID NO: 246 | NWVPQIEHW | 19.33% |
| SEQ ID NO: 247 | VAILGLNPT | 19.31% |
| SEQ ID NO: 248 | ANVPMCSLQ | 19.31% |
| SEQ ID NO: 249 | LIPMDWIGM | 19.30% |
| SEQ ID NO: 250 | IYCGNSDMQ | 18.73% |
| SEQ ID NO: 251 | PAETLMDGF | 18.71% |
| SEQ ID NO: 252 | FSYQIQCQN | 18.43% |
| SEQ ID NO: 253 | TMRLFWDMG | 18.42% |
| SEQ ID NO: 254 | QRVPMQLVE | 18.42% |
| SEQ ID NO: 255 | AMTRQNGWM | 18.16% |
| SEQ ID NO: 256 | GMRTHSVMN | 18.13% |
| SEQ ID NO: 257 | LITVQHIRN | 18.13% |
| SEQ ID NO: 258 | TTYPHNTHG | 17.88% |
| SEQ ID NO: 259 | RIEMDEWEA | 17.85% |
| SEQ ID NO: 260 | DNTPEHCYM | 17.57% |
| SEQ ID NO: 261 | SSVVMAKQP | 17.55% |
| SEQ ID NO: 262 | CLVRIQKDH | 17.54% |
| SEQ ID NO: 263 | TLPKMHMWP | 17.54% |
| SEQ ID NO: 264 | MPHHSPDCW | 17.32% |
| SEQ ID NO: 265 | QELECHACS | 17.31% |
| SEQ ID NO: 266 | HMHNLPVKP | 17.31% |
| SEQ ID NO: 267 | GIAVWFDFQ | 17.25% |
| SEQ ID NO: 268 | ANRSRPTWQ | 17.25% |
| SEQ ID NO: 269 | KTFPILEAW | 17.25% |
| SEQ ID NO: 270 | ALQYCQGKD | 16.97% |
| SEQ ID NO: 271 | YHFEYMTSF | 16.97% |
| SEQ ID NO: 272 | VCGHRNDGN | 16.96% |
| SEQ ID NO: 273 | EICAAMDSF | 16.68% |
| SEQ ID NO: 274 | YWFVLNPCV | 16.41% |
| SEQ ID NO: 275 | QQNPCLGGM | 16.32% |
| SEQ ID NO: 276 | FNWFVRAYI | 16.14% |
| SEQ ID NO: 277 | DCDPYNMVD | 16.12% |
| SEQ ID NO: 278 | RTGDAGMSP | 16.10% |
| SEQ ID NO: 279 | LEFNGHLFN | 16.08% |
| SEQ ID NO: 280 | VYVAAGGGC | 15.92% |
| SEQ ID NO: 281 | MTTGLMAYE | 15.87% |
| SEQ ID NO: 282 | MAIDNLECK | 15.83% |
| SEQ ID NO: 283 | QKAIVAQND | 15.81% |
| SEQ ID NO: 284 | GGDGRVHNK | 15.52% |
| SEQ ID NO: 285 | THDAARKTA | 15.50% |
| SEQ ID NO: 286 | TWNEPHQNQ | 15.20% |
| SEQ ID NO: 287 | ANRADWQHL | 14.96% |
| SEQ ID NO: 288 | CTKAYRKPG | 14.93% |
| SEQ ID NO: 289 | RTAVIQCIE | 14.92% |
| SEQ ID NO: 290 | WKVSPIQGF | 14.92% |
| SEQ ID NO: 291 | MCGEYQPGS | 14.91% |
| SEQ ID NO: 292 | NHHPYTFHQ | 14.91% |
| SEQ ID NO: 293 | NPRYCLSLS | 14.64% |
| SEQ ID NO: 294 | SRASQQSMW | 14.64% |
| SEQ ID NO: 295 | VLKSCDRHG | 14.64% |
| SEQ ID NO: 296 | KEARSQTGD | 14.63% |
| SEQ ID NO: 297 | FANGCTGSI | 14.63% |
| SEQ ID NO: 298 | GAMAAPYHD | 14.62% |
| SEQ ID NO: 299 | YEHPIMWPV | 14.62% |
| SEQ ID NO: 300 | CYGWTGCKC | 14.33% |
| SEQ ID NO: 301 | AAAMRDKTD | 14.33% |
| SEQ ID NO: 302 | KLRIGVVCN | 14.33% |
| SEQ ID NO: 303 | TPHSIHTDK | 14.33% |
| SEQ ID NO: 304 | QTETWEFGA | 14.26% |
| SEQ ID NO: 305 | KAEMKMGCN | 14.16% |
| SEQ ID NO: 306 | VPICCNLPM | 14.11% |
| SEQ ID NO: 307 | LPSHCNLGS | 14.05% |
| SEQ ID NO: 308 | SMNRTHLER | 14.04% |
| SEQ ID NO: 309 | FGEFTMDEN | 13.84% |
| SEQ ID NO: 310 | YTAHNANVV | 13.78% |
| SEQ ID NO: 311 | GTMKHNGRA | 13.76% |
| SEQ ID NO: 312 | EHYCKDNVL | 13.75% |
| SEQ ID NO: 313 | TKRMMEDSG | 13.75% |
| SEQ ID NO: 314 | CERDCNCTA | 13.74% |
| SEQ ID NO: 315 | TGRVTADMA | 13.74% |
| SEQ ID NO: 316 | YNGDRLFCF | 13.53% |
| SEQ ID NO: 317 | NHEVVAFMQ | 13.47% |
| SEQ ID NO: 318 | QNEICVHHE | 13.47% |
| SEQ ID NO: 319 | LRSGMIVSG | 13.46% |
| SEQ ID NO: 320 | QPKMWMMAS | 13.45% |
| SEQ ID NO: 321 | DVRHSWSEK | 13.45% |
| SEQ ID NO: 322 | EILQDVYYL | 13.45% |
| SEQ ID NO: 323 | SVVNNLKAS | 13.45% |
| SEQ ID NO: 324 | RCTDCSYFY | 13.29% |
| SEQ ID NO: 325 | GSTSRNQRD | 13.17% |
| SEQ ID NO: 326 | LDCNAMPST | 13.16% |
| SEQ ID NO: 327 | QGRWACWYS | 13.16% |
| SEQ ID NO: 328 | TIEWYPDSQ | 13.16% |
| SEQ ID NO: 329 | KAVSYHHDG | 13.16% |
| SEQ ID NO: 330 | TMEHCQRPQ | 13.16% |
| SEQ ID NO: 331 | RMFWDTSDR | 12.89% |
| SEQ ID NO: 332 | MCNPKMMSS | 12.88% |
| SEQ ID NO: 333 | DSHDMFVYK | 12.87% |
| SEQ ID NO: 334 | LTCYHNELS | 12.87% |
| SEQ ID NO: 335 | AVVEHAVNR | 12.87% |
| SEQ ID NO: 336 | CGQMVICSE | 12.87% |
| SEQ ID NO: 337 | NLRDAHWCM | 12.87% |
| SEQ ID NO: 338 | ESSQEIKTC | 12.77% |
| SEQ ID NO: 339 | AKMWQLENP | 12.73% |
| SEQ ID NO: 340 | GTIPKQHEF | 12.59% |
| SEQ ID NO: 341 | ARDCYWKAR | 12.58% |
| SEQ ID NO: 342 | AADREQGWW | 12.57% |
| SEQ ID NO: 343 | DSVQQRWYV | 12.57% |
| SEQ ID NO: 344 | KCGQDWIHG | 12.33% |
| SEQ ID NO: 345 | QVYAPEIEG | 12.32% |
| SEQ ID NO: 346 | CCCVPRSIY | 12.29% |
| SEQ ID NO: 347 | AAHGEMKTA | 12.28% |
| SEQ ID NO: 348 | QFWLMHEWP | 12.28% |
| SEQ ID NO: 349 | KFAMDWVSD | 12.00% |
| SEQ ID NO: 350 | TSDSVINWL | 12.00% |
| SEQ ID NO: 351 | QIIYRCRST | 12.00% |
| SEQ ID NO: 352 | GYDLRGADE | 11.99% |
| SEQ ID NO: 353 | CIRFMMREG | 11.99% |
| SEQ ID NO: 354 | HSNTHGYPL | 11.73% |
| SEQ ID NO: 355 | HGNMPHCYP | 11.72% |
| SEQ ID NO: 356 | VGLSIKMYG | 11.71% |
| SEQ ID NO: 357 | ESWRYKHQA | 11.70% |
| SEQ ID NO: 358 | PKRYQAWTW | 11.70% |
| SEQ ID NO: 359 | SCSQKLMMQ | 11.70% |
| SEQ ID NO: 360 | ALVTYRSMQ | 11.70% |
| SEQ ID NO: 361 | ALNSDMAAW | 11.60% |
| SEQ ID NO: 362 | NHSLCWDSK | 11.60% |
| SEQ ID NO: 363 | KKEDYAKFF | 11.41% |
| SEQ ID NO: 364 | RQYLYKRQQ | 11.41% |
| SEQ ID NO: 365 | TAMPEHRWD | 11.41% |
| SEQ ID NO: 366 | GFCTIPVAD | 11.40% |
| SEQ ID NO: 367 | IAVQKRLFC | 11.40% |
| SEQ ID NO: 368 | LAEIKDWVP | 11.40% |
| SEQ ID NO: 369 | MLPMFMGQN | 11.40% |
| SEQ ID NO: 370 | NVRTYIDDS | 11.40% |
| SEQ ID NO: 371 | YFWISKALY | 11.13% |
| SEQ ID NO: 372 | ISYQPNPME | 11.12% |
| SEQ ID NO: 373 | ACDDWCWMC | 11.11% |
| SEQ ID NO: 374 | QTEMKKCAR | 11.11% |
| SEQ ID NO: 375 | ATTHKVKDN | 11.11% |
| SEQ ID NO: 376 | DTFKCMNVS | 11.11% |
| SEQ ID NO: 377 | QSFSYDAKE | 11.11% |
| SEQ ID NO: 378 | VIGCQQGLG | 11.11% |
| SEQ ID NO: 379 | YGPEILKLT | 11.11% |
| SEQ ID NO: 380 | WAAQMYQQE | 11.09% |
| SEQ ID NO: 381 | DIACLDQWQ | 10.93% |
| SEQ ID NO: 382 | TSLVMPSLP | 10.89% |
| SEQ ID NO: 383 | MHSQQRAIK | 10.84% |
| SEQ ID NO: 384 | VRQSVPHWA | 10.83% |
| SEQ ID NO: 385 | DTRPSRDSS | 10.82% |
| SEQ ID NO: 386 | RSGAKCYCD | 10.82% |
| SEQ ID NO: 387 | ECKTHRDVY | 10.63% |
| SEQ ID NO: 388 | DFLHVTSIM | 10.55% |
| SEQ ID NO: 389 | QTKFMDDMH | 10.54% |
| SEQ ID NO: 390 | TSMTERRTV | 10.54% |
| SEQ ID NO: 391 | WTTWAIPCC | 10.53% |
| SEQ ID NO: 392 | LPTLRVADK | 10.53% |
| SEQ ID NO: 393 | KTGTTMEPM | 10.52% |
| SEQ ID NO: 394 | TKQFVHNED | 10.50% |
| SEQ ID NO: 395 | TCQEPHNIT | 10.28% |
| SEQ ID NO: 396 | QKVASMGCW | 10.27% |
| SEQ ID NO: 397 | CKGHDAGEY | 10.27% |
| SEQ ID NO: 398 | FPWSRPKCW | 10.26% |
| SEQ ID NO: 399 | HAMSLSTFQ | 10.25% |
| SEQ ID NO: 400 | MTCVWHSDF | 10.25% |
| SEQ ID NO: 401 | VKAWWHDHQ | 10.24% |
| SEQ ID NO: 402 | DVLKCFINE | 10.24% |
| SEQ ID NO: 403 | PIKGVAVQE | 10.24% |
| SEQ ID NO: 404 | LSRLYPNTY | 10.23% |
| SEQ ID NO: 405 | KQTVMTDTS | 9.96% |
| SEQ ID NO: 406 | SATPHWYCH | 9.95% |
| SEQ ID NO: 407 | KTTDLFQYE | 9.94% |
| SEQ ID NO: 408 | VTWINLRSG | 9.92% |
| SEQ ID NO: 409 | IHQLVGAFW | 9.66% |
| SEQ ID NO: 410 | CVNLMAVNN | 9.66% |
| SEQ ID NO: 411 | EKQKLTVSS | 9.66% |
| SEQ ID NO: 412 | RLDHWATCD | 9.65% |
| SEQ ID NO: 413 | AVEFWLERM | 9.65% |
| SEQ ID NO: 414 | KTNNTVCHS | 9.65% |
| SEQ ID NO: 415 | HHLRNVHHC | 9.44% |
| SEQ ID NO: 416 | ARGTCNYCY | 9.37% |
| SEQ ID NO: 417 | STEENEALY | 9.37% |
| SEQ ID NO: 418 | KKQQISIRL | 9.36% |
| SEQ ID NO: 419 | SAAEKAMDQ | 9.36% |
| SEQ ID NO: 420 | TAPGGSTPQ | 9.36% |
| SEQ ID NO: 421 | QTNQVVVVE | 9.36% |
| SEQ ID NO: 422 | DSTPNLFDP | 9.08% |
| SEQ ID NO: 423 | DVASYCQLQ | 9.08% |
| SEQ ID NO: 424 | TQCKQLTVM | 9.08% |
| SEQ ID NO: 425 | TMEHHECMW | 9.07% |
| SEQ ID NO: 426 | DVLVCEILW | 9.07% |
| SEQ ID NO: 427 | NIYSGINNP | 9.07% |
| SEQ ID NO: 428 | MFPKKMCLH | 8.83% |
| SEQ ID NO: 429 | THQNTWLFY | 8.83% |
| SEQ ID NO: 430 | NHGYRNNSM | 8.78% |
| SEQ ID NO: 431 | QMHICMYEG | 8.74% |
| SEQ ID NO: 432 | KWEVMSAAY | 8.73% |
| SEQ ID NO: 433 | QRMIVDALQ | 8.50% |
| SEQ ID NO: 434 | TMGVSSYSM | 8.50% |
| SEQ ID NO: 435 | TGAGIKEAH | 8.49% |
| SEQ ID NO: 436 | VPWVEFRLQ | 8.49% |
| SEQ ID NO: 437 | QPWPHLINK | 8.48% |
| SEQ ID NO: 438 | SSLCRAYDN | 8.22% |
| SEQ ID NO: 439 | CVKSRQDSD | 8.21% |
| SEQ ID NO: 440 | NYILTQLGM | 8.20% |
| SEQ ID NO: 441 | FGGVEDSWM | 8.19% |
| SEQ ID NO: 442 | SVKDPMDWG | 8.19% |
| SEQ ID NO: 443 | YSDRFQFQF | 8.19% |
| SEQ ID NO: 444 | NCPMYDMIS | 8.12% |
| SEQ ID NO: 445 | IWGIYVHYF | 8.08% |
| SEQ ID NO: 446 | FTPELWENY | 7.91% |
| SEQ ID NO: 447 | AHSRMDWTP | 7.91% |
| SEQ ID NO: 448 | QPFQRKVEE | 7.91% |
| SEQ ID NO: 449 | DYENCFESY | 7.91% |
| SEQ ID NO: 450 | IEVYHFTNS | 7.89% |
| SEQ ID NO: 451 | KALTHDARN | 7.89% |
| SEQ ID NO: 452 | KTQMKRLRM | 7.70% |
| SEQ ID NO: 453 | KAPWAPMDS | 7.67% |
| SEQ ID NO: 454 | ETVMMPMSI | 7.64% |
| SEQ ID NO: 455 | DYVDQCQVS | 7.63% |
| SEQ ID NO: 456 | RACVGSPLW | 7.62% |
| SEQ ID NO: 457 | KIMHSWYND | 7.62% |
| SEQ ID NO: 458 | RHEPTQSVS | 7.62% |
| SEQ ID NO: 459 | DPKGQSIQA | 7.61% |
| SEQ ID NO: 460 | RTTSRDVFD | 7.61% |
| SEQ ID NO: 461 | QCMVMTEAD | 7.61% |
| SEQ ID NO: 462 | VQHNSESNI | 7.61% |
| SEQ ID NO: 463 | WHESTTIGS | 7.61% |
| SEQ ID NO: 464 | RHEQYVVAT | 7.60% |
| SEQ ID NO: 465 | VVCTDTRAL | 7.49% |
| SEQ ID NO: 466 | EHHAMMLHE | 7.47% |
| SEQ ID NO: 467 | KACMSHQGR | 7.42% |
| SEQ ID NO: 468 | DSPAYTTQY | 7.35% |
| SEQ ID NO: 469 | ICELTHCNY | 7.33% |
| SEQ ID NO: 470 | KATDLMVNG | 7.32% |
| SEQ ID NO: 471 | PQSHEKQLL | 7.32% |
| SEQ ID NO: 472 | CHCHYNPFC | 7.32% |
| SEQ ID NO: 473 | EAECVAGER | 7.32% |
| SEQ ID NO: 474 | EFWTTHSKG | 7.31% |
| SEQ ID NO: 475 | GHVLLGWYP | 7.31% |
| SEQ ID NO: 476 | YCQQLFLCD | 7.05% |
| SEQ ID NO: 477 | AKEPVRLYP | 7.04% |
| SEQ ID NO: 478 | EERLFTGCH | 7.04% |
| SEQ ID NO: 479 | QTSPKFCMC | 7.04% |
| SEQ ID NO: 480 | WMNQCGCKD | 7.04% |
| SEQ ID NO: 481 | NYSRGKENE | 7.03% |
| SEQ ID NO: 482 | DIQRWEHEG | 7.02% |
| SEQ ID NO: 483 | MMAMKQGFY | 7.02% |
| SEQ ID NO: 484 | KVNACGNLT | 6.89% |
| SEQ ID NO: 485 | ETTHRREWS | 6.75% |
| SEQ ID NO: 486 | PTVEEVVVQ | 6.75% |
| SEQ ID NO: 487 | SIEPRKTVG | 6.75% |
| SEQ ID NO: 488 | QTIMRHAQT | 6.74% |
| SEQ ID NO: 489 | RIETVSVPL | 6.74% |
| SEQ ID NO: 490 | SRTICVHGN | 6.74% |
| SEQ ID NO: 491 | GACKYSMCL | 6.73% |
| SEQ ID NO: 492 | GWDQNCKDS | 6.73% |
| SEQ ID NO: 493 | ASINCHLTE | 6.44% |
| SEQ ID NO: 494 | HYMELLCGC | 6.44% |
| SEQ ID NO: 495 | ITRTYQEMQ | 6.44% |
| SEQ ID NO: 496 | RANENHPFT | 6.44% |
| SEQ ID NO: 497 | LNKMQSYSA | 6.42% |
| SEQ ID NO: 498 | NGYLVHACQ | 6.21% |
| SEQ ID NO: 499 | LMAYCYANN | 6.18% |
| SEQ ID NO: 500 | KESKEHRWA | 6.16% |
| SEQ ID NO: 501 | FTTMCEHTA | 6.15% |
| SEQ ID NO: 502 | ATMWVQAYG | 6.15% |
| SEQ ID NO: 503 | RGQFHLIMD | 6.15% |
| SEQ ID NO: 504 | DLVDLRSWM | 6.14% |
| SEQ ID NO: 505 | LGAVLNNMV | 6.14% |
| SEQ ID NO: 506 | MYRQKEMQT | 6.14% |
| SEQ ID NO: 507 | RLEDTVGGS | 6.14% |
| SEQ ID NO: 508 | ACHYRLDNC | 6.06% |
| SEQ ID NO: 509 | LNNAGYRPC | 6.02% |
| SEQ ID NO: 510 | SFSHTDLEA | 5.89% |
| SEQ ID NO: 511 | ATEPLKFQY | 5.87% |
| SEQ ID NO: 512 | NQNPYDMMG | 5.87% |
| SEQ ID NO: 513 | FKIEWSQDI | 5.86% |
| SEQ ID NO: 514 | QIGHQYKWD | 5.86% |
| SEQ ID NO: 515 | SWSQLQHTD | 5.86% |
| SEQ ID NO: 516 | QMMDFQHPA | 5.86% |
| SEQ ID NO: 517 | LFTQRGWGN | 5.85% |
| SEQ ID NO: 518 | TATQREAFV | 5.59% |
| SEQ ID NO: 519 | KENPSEYEM | 5.58% |
| SEQ ID NO: 520 | KEFQLQMEP | 5.57% |
| SEQ ID NO: 521 | KSAGIRTLL | 5.57% |
| SEQ ID NO: 522 | MSEKGYAWV | 5.57% |
| SEQ ID NO: 523 | VYCGKDFQA | 5.57% |
| SEQ ID NO: 524 | CVKLVMANC | 5.56% |
| SEQ ID NO: 525 | ALRPYMDDR | 5.56% |
| SEQ ID NO: 526 | CSIQINNCR | 5.56% |
| SEQ ID NO: 527 | GTRINTCNG | 5.56% |
| SEQ ID NO: 528 | FGAVELDAP | 5.56% |
| SEQ ID NO: 529 | YCQRGHLSC | 5.56% |
| SEQ ID NO: 530 | HRMNNHAWF | 5.54% |
| SEQ ID NO: 531 | NADEKYNNT | 5.52% |
| SEQ ID NO: 532 | MGATKSDDF | 5.32% |
| SEQ ID NO: 533 | SMHDYNFNV | 5.31% |
| SEQ ID NO: 534 | GNECFHNVE | 5.29% |
| SEQ ID NO: 535 | GVWMPHGWR | 5.29% |
| SEQ ID NO: 536 | EIWPAAWMD | 5.28% |
| SEQ ID NO: 537 | APKWQIVDP | 5.27% |
| SEQ ID NO: 538 | HAMHWWSPM | 5.27% |
| SEQ ID NO: 539 | QCKYSERGT | 5.21% |
| SEQ ID NO: 540 | TCCMWTFIW | 5.01% |
| SEQ ID NO: 541 | ETGYRENPC | 5.00% |
| SEQ ID NO: 542 | MEMFTMKSC | 4.99% |
| SEQ ID NO: 543 | PQGFCKPQC | 4.98% |
| SEQ ID NO: 544 | KTTFDVACY | 4.98% |
| SEQ ID NO: 545 | WCRTPQREN | 4.98% |
| SEQ ID NO: 546 | GSQQMQASQ | 4.97% |
| SEQ ID NO: 547 | ITGSPQNGC | 4.97% |
| SEQ ID NO: 548 | GTVEAPQSF | 4.93% |
| SEQ ID NO: 549 | LNCPSKDET | 4.85% |
| SEQ ID NO: 550 | CSNTETHGV | 4.84% |
| SEQ ID NO: 551 | QMEQWHRGG | 4.78% |
| SEQ ID NO: 552 | IPFNSMIYC | 4.72% |
| SEQ ID NO: 553 | RVEQWRGQQ | 4.70% |
| SEQ ID NO: 554 | WNMPWGFYH | 4.70% |
| SEQ ID NO: 555 | MISCMDSWN | 4.69% |
| SEQ ID NO: 556 | TSRALDVAA | 4.69% |
| SEQ ID NO: 557 | FFRWRWGLG | 4.69% |
| SEQ ID NO: 558 | FPKCAIWHY | 4.69% |
| SEQ ID NO: 559 | NMIKRCSQG | 4.69% |
| SEQ ID NO: 560 | GTSVNTLMM | 4.68% |
| SEQ ID NO: 561 | MRQRTYICQ | 4.68% |
| SEQ ID NO: 562 | RGCMAECMP | 4.68% |
| SEQ ID NO: 563 | MPMPVYQRD | 4.59% |
| SEQ ID NO: 564 | ALNKTLAES | 4.55% |
| SEQ ID NO: 565 | EAGMWPSKA | 4.41% |
| SEQ ID NO: 566 | AAVKEQQVM | 4.40% |
| SEQ ID NO: 567 | SAQYSGFDT | 4.40% |
| SEQ ID NO: 568 | LILDFNKID | 4.40% |
| SEQ ID NO: 569 | MSAVSLGRS | 4.39% |
| SEQ ID NO: 570 | RWGSPMAVP | 4.39% |
| SEQ ID NO: 571 | KTVTNGDAG | 4.39% |
| SEQ ID NO: 572 | YTMSMQMQR | 4.39% |
| SEQ ID NO: 573 | AHEHYRIFP | 4.11% |
| SEQ ID NO: 574 | GKFPMPTHS | 4.11% |
| SEQ ID NO: 575 | KIENRGQCR | 4.10% |
| SEQ ID NO: 576 | MWEMRRAMG | 4.10% |
| SEQ ID NO: 577 | HGSSYNTVQ | 4.09% |
| SEQ ID NO: 578 | VIKAFQHFN | 4.09% |
| SEQ ID NO: 579 | EVRCNKCRS | 3.88% |
| SEQ ID NO: 580 | LALQFQYNS | 3.85% |
| SEQ ID NO: 581 | GVITVDAKG | 3.84% |
| SEQ ID NO: 582 | CLHHHYPVT | 3.83% |
| SEQ ID NO: 583 | AFCRGHYQA | 3.82% |
| SEQ ID NO: 584 | KTGWDSHWS | 3.82% |
| SEQ ID NO: 585 | RYEMMDTSD | 3.82% |
| SEQ ID NO: 586 | DIFWSSMDF | 3.81% |
| SEQ ID NO: 587 | MSTHVLKWV | 3.81% |
| SEQ ID NO: 588 | CDVWWFPNL | 3.81% |
| SEQ ID NO: 589 | IVFHNKFQH | 3.81% |
| SEQ ID NO: 590 | SHIQSASNL | 3.81% |
| SEQ ID NO: 591 | VKQLRQDGF | 3.81% |
| SEQ ID NO: 592 | DKCLNNKFY | 3.80% |
| SEQ ID NO: 593 | RNDWQQVFS | 3.80% |
| SEQ ID NO: 594 | QRREQCTST | 3.77% |
| SEQ ID NO: 595 | YHWSYEGAT | 3.75% |
| SEQ ID NO: 596 | TDFDKRQVP | 3.67% |
| SEQ ID NO: 597 | QISMKGEHG | 3.55% |
| SEQ ID NO: 598 | LVGERKNAY | 3.53% |
| SEQ ID NO: 599 | DPMKIFVSH | 3.53% |
| SEQ ID NO: 600 | KQTRTDYAY | 3.52% |
| SEQ ID NO: 601 | AVAMIRPNQ | 3.51% |
| SEQ ID NO: 602 | ESTHMLYEM | 3.51% |
| SEQ ID NO: 603 | VDADYCSCS | 3.51% |
| SEQ ID NO: 604 | EMVRMQRGT | 3.51% |
| SEQ ID NO: 605 | NCDHRFGTL | 3.50% |
| SEQ ID NO: 606 | SVVFSAAEV | 3.48% |
| SEQ ID NO: 607 | NARPMCGQV | 3.40% |
| SEQ ID NO: 608 | TEATEMTYY | 3.33% |
| SEQ ID NO: 609 | AYGSFEKDW | 3.24% |
| SEQ ID NO: 610 | MNSFYRAEW | 3.24% |
| SEQ ID NO: 611 | MIDNCKICL | 3.23% |
| SEQ ID NO: 612 | VCRGSPLAE | 3.23% |
| SEQ ID NO: 613 | DTRMDQCCY | 3.22% |
| SEQ ID NO: 614 | LVDCSNTCQ | 3.22% |
| SEQ ID NO: 615 | INKNMDAAY | 3.22% |
| SEQ ID NO: 616 | TGMTFNSFR | 3.22% |
| SEQ ID NO: 617 | AWKIAHNSF | 3.22% |
| SEQ ID NO: 618 | NFMQMSIMG | 3.22% |
| SEQ ID NO: 619 | WIKHCWPPH | 3.22% |
| SEQ ID NO: 620 | NLWNIAMQQ | 3.18% |
| SEQ ID NO: 621 | EHMDRADDA | 3.11% |
| SEQ ID NO: 622 | AQDVYYPGS | 3.08% |
| SEQ ID NO: 623 | FPQTMHAQA | 2.96% |
| SEQ ID NO: 624 | TFGCIESMQ | 2.95% |
| SEQ ID NO: 625 | KFFYTELMH | 2.94% |
| SEQ ID NO: 626 | GRLGPGTWK | 2.93% |
| SEQ ID NO: 627 | ASTDGTWKD | 2.92% |
| SEQ ID NO: 628 | DARPFLAWM | 2.92% |
| SEQ ID NO: 629 | KFHSKERMN | 2.91% |
| SEQ ID NO: 630 | SLQHFPECA | 2.87% |
| SEQ ID NO: 631 | VCVSAHHHF | 2.87% |
| SEQ ID NO: 632 | AVIFELDAT | 2.84% |
| SEQ ID NO: 633 | EQADQMQVD | 2.83% |
| SEQ ID NO: 634 | MSQFKAYLF | 2.82% |
| SEQ ID NO: 635 | EMSIQSSLG | 2.76% |
| SEQ ID NO: 636 | GTGYRTFIG | 2.67% |
| SEQ ID NO: 637 | KASHTVTGT | 2.65% |
| SEQ ID NO: 638 | NSVALENRG | 2.64% |
| SEQ ID NO: 639 | NEGPLMCWF | 2.64% |
| SEQ ID NO: 640 | NNELQSRPK | 2.63% |
| SEQ ID NO: 641 | NGTQCLNMD | 2.52% |
| SEQ ID NO: 642 | SSTWQNVDK | 2.51% |
| SEQ ID NO: 643 | YMVPYSNYP | 2.50% |
| SEQ ID NO: 644 | WFDRPMCMF | 2.48% |
| SEQ ID NO: 645 | LECTALVAM | 2.38% |
| SEQ ID NO: 646 | QVQSGQFIF | 2.37% |
| SEQ ID NO: 647 | KMETMICFD | 2.37% |
| SEQ ID NO: 648 | QSDAVHEPA | 2.36% |
| SEQ ID NO: 649 | GYMCEDAGI | 2.35% |
| SEQ ID NO: 650 | EIHPGDLND | 2.35% |
| SEQ ID NO: 651 | EFSKACTNE | 2.35% |
| SEQ ID NO: 652 | KSTYYNLKW | 2.34% |
| SEQ ID NO: 653 | TSSRTNPWG | 2.34% |
| SEQ ID NO: 654 | SFKGFHLQN | 2.28% |
| SEQ ID NO: 655 | CDEWDNLTH | 2.20% |
| SEQ ID NO: 656 | DAGIGMKFY | 2.16% |
| SEQ ID NO: 657 | LGDQAGYHV | 2.14% |
| SEQ ID NO: 658 | IIVWMMMYR | 2.13% |
| SEQ ID NO: 659 | GADEWLLTM | 2.10% |
| SEQ ID NO: 660 | QSYPTMYQC | 2.09% |
| SEQ ID NO: 661 | DRTWTMTQM | 2.08% |
| SEQ ID NO: 662 | TSWTTEHRM | 2.06% |
| SEQ ID NO: 663 | TLRICELHG | 2.06% |
| SEQ ID NO: 664 | CQASHEHAH | 2.06% |
| SEQ ID NO: 665 | ESAEPRMIP | 2.06% |
| SEQ ID NO: 666 | FWMQLWCLP | 2.03% |
| SEQ ID NO: 667 | QGYCRNCTG | 2.02% |
| SEQ ID NO: 668 | ESCLVRRGW | 2.00% |
| SEQ ID NO: 669 | QIRCTCGWP | 1.98% |
| SEQ ID NO: 670 | HAYQHDHSS | 1.97% |
| SEQ ID NO: 671 | DMAVSEAHC | 1.97% |
| SEQ ID NO: 672 | WAGMAYHAQ | 1.94% |
| SEQ ID NO: 673 | FQQDIQIGQ | 1.92% |
| SEQ ID NO: 674 | VVHKGRWKE | 1.91% |
| SEQ ID NO: 675 | MQLRQSDKW | 1.89% |
| SEQ ID NO: 676 | YVRQFPIEL | 1.87% |
| SEQ ID NO: 677 | IIHRTEGAA | 1.87% |
| SEQ ID NO: 678 | GQYWGQGWL | 1.85% |
| SEQ ID NO: 679 | GSTHMNQEG | 1.85% |
| SEQ ID NO: 680 | INAGDKPNT | 1.83% |
| SEQ ID NO: 681 | LGTAMREMQ | 1.81% |
| SEQ ID NO: 682 | PGGGDRPAC | 1.81% |
| SEQ ID NO: 683 | QDVQMWYQI | 1.80% |
| SEQ ID NO: 684 | QARAVMTAD | 1.79% |
| SEQ ID NO: 685 | GAHDPKVWY | 1.78% |
| SEQ ID NO: 686 | GTLGYHITG | 1.77% |
| SEQ ID NO: 687 | SMLMMITGM | 1.77% |
| SEQ ID NO: 688 | RDDYGIYDN | 1.77% |
| SEQ ID NO: 689 | HLVTTRHSH | 1.76% |
| SEQ ID NO: 690 | ITDLRCWLK | 1.76% |
| SEQ ID NO: 691 | IMVRELMSS | 1.75% |
| SEQ ID NO: 692 | NAEHCFMGQ | 1.73% |
| SEQ ID NO: 693 | NNLQWMYAK | 1.73% |
| SEQ ID NO: 694 | SPMPFLNVL | 1.72% |
| SEQ ID NO: 695 | CEQQLIAEG | 1.72% |
| SEQ ID NO: 696 | NDKYAFADR | 1.71% |
| SEQ ID NO: 697 | WIKRHIPSG | 1.71% |
| SEQ ID NO: 698 | REGAKHCVN | 1.69% |
| SEQ ID NO: 699 | CDDWTFERQ | 1.69% |
| SEQ ID NO: 700 | GVGPSHGER | 1.69% |
| SEQ ID NO: 701 | ESTFWQHTF | 1.69% |
| SEQ ID NO: 702 | MFVCCTIIV | 1.67% |
| SEQ ID NO: 703 | QKYEPRFFG | 1.67% |
| SEQ ID NO: 704 | AHVKIQLSW | 1.66% |
| SEQ ID NO: 705 | TFSHMLSVE | 1.66% |
| SEQ ID NO: 706 | LRPLQHIQL | 1.65% |
| SEQ ID NO: 707 | MANGNWGVW | 1.65% |
| SEQ ID NO: 708 | VTDAAMFMG | 1.64% |
| SEQ ID NO: 709 | WHEEKPANS | 1.62% |
| SEQ ID NO: 710 | KCQRMSVQQ | 1.62% |
| SEQ ID NO: 711 | DDRIGHCRN | 1.62% |
| SEQ ID NO: 712 | IAPEARCGT | 1.62% |
| SEQ ID NO: 713 | GCSSSVNMR | 1.61% |
| SEQ ID NO: 714 | RKRVRAYSE | 1.61% |
| SEQ ID NO: 715 | QEPKEDHIV | 1.60% |
| SEQ ID NO: 716 | SILPYPIDV | 1.59% |
| SEQ ID NO: 717 | ILDIINTET | 1.59% |
| SEQ ID NO: 718 | RFMLESWLH | 1.59% |
| SEQ ID NO: 719 | NSAHVHYFH | 1.58% |
| SEQ ID NO: 720 | LVKGHMTTS | 1.58% |
| SEQ ID NO: 721 | DKYANVHNE | 1.57% |
| SEQ ID NO: 722 | RYCTPDHEV | 1.57% |
| SEQ ID NO: 723 | RYHRRHDKP | 1.57% |
| SEQ ID NO: 724 | SMRMFEHSC | 1.56% |
| SEQ ID NO: 725 | SRCNKPTVR | 1.56% |
| SEQ ID NO: 726 | TFHQSDYWE | 1.55% |
| SEQ ID NO: 727 | VLQMPWLYT | 1.55% |
| SEQ ID NO: 728 | VCTPPLNMQ | 1.55% |
| SEQ ID NO: 729 | LNSDRLIEY | 1.52% |
| SEQ ID NO: 730 | CETYVPVIY | 1.52% |
| SEQ ID NO: 731 | WVPTFHARY | 1.52% |
| SEQ ID NO: 732 | CEIRTYEWS | 1.52% |
| SEQ ID NO: 733 | KHDGFSVVG | 1.51% |
| SEQ ID NO: 734 | AWYHTQDDM | 1.50% |
| SEQ ID NO: 735 | WMCEFSCIQ | 1.50% |
| SEQ ID NO: 736 | AMVMSKTES | 1.49% |
| SEQ ID NO: 737 | NFCCLECCI | 1.49% |
| SEQ ID NO: 738 | RTSPALLCA | 1.49% |
| SEQ ID NO: 739 | DITLWMATE | 1.48% |
| SEQ ID NO: 740 | AVAEIRPEP | 1.47% |
| SEQ ID NO: 741 | SARVTTMLY | 1.47% |
| SEQ ID NO: 742 | TLRAEMSFH | 1.47% |
| SEQ ID NO: 743 | ASKQNGINC | 1.47% |
| SEQ ID NO: 744 | RRCPALTIR | 1.47% |
| SEQ ID NO: 745 | ARPLVCQAL | 1.46% |
| SEQ ID NO: 746 | MMLGYMGQD | 1.46% |
| SEQ ID NO: 747 | QEHIYKHSN | 1.46% |
| SEQ ID NO: 748 | DQNTFPETG | 1.46% |
| SEQ ID NO: 749 | KSEGWLLGD | 1.45% |
| SEQ ID NO: 750 | SPCFDVFEE | 1.44% |
| SEQ ID NO: 751 | TINKFPWVC | 1.44% |
| SEQ ID NO: 752 | IRFTTQIVD | 1.44% |
| SEQ ID NO: 753 | SVWEKTQMR | 1.43% |
| SEQ ID NO: 754 | CPCHKWASC | 1.43% |
| SEQ ID NO: 755 | HDHAAKQLD | 1.43% |
| SEQ ID NO: 756 | RPVGKLYMI | 1.43% |
| SEQ ID NO: 757 | MWGCKLFVC | 1.42% |
| SEQ ID NO: 758 | KHNPSRHDI | 1.42% |
| SEQ ID NO: 759 | NPYQCIVAG | 1.39% |
| SEQ ID NO: 760 | CMRGGQKLT | 1.36% |
| SEQ ID NO: 761 | DVDTSQFDR | 1.34% |
| SEQ ID NO: 762 | VCVKWRNVN | 1.34% |
| SEQ ID NO: 763 | WASPSVWRR | 1.34% |
| SEQ ID NO: 764 | YFHYRYDYG | 1.33% |
| SEQ ID NO: 765 | KVYYIISHQ | 1.32% |
| SEQ ID NO: 766 | QTYRGDWQK | 1.31% |
| SEQ ID NO: 767 | RVDKAGAIF | 1.28% |
| SEQ ID NO: 768 | VFNLMLQDK | 1.27% |
| SEQ ID NO: 769 | VKKPQYDMH | 1.26% |
| SEQ ID NO: 770 | QYVQCAAKD | 1.23% |
| SEQ ID NO: 771 | EGCYFWKQV | 1.22% |
| SEQ ID NO: 772 | TMKTWIYRN | 1.22% |
| SEQ ID NO: 773 | GWESFPHCG | 1.21% |
| SEQ ID NO: 774 | QKAEHLFVG | 1.21% |
| SEQ ID NO: 775 | PDSIPNSWC | 1.21% |
| SEQ ID NO: 776 | CHGRMLCYI | 1.20% |
| SEQ ID NO: 777 | NQQRTYNSG | 1.19% |
| SEQ ID NO: 778 | LDTKTLANS | 1.19% |
| SEQ ID NO: 779 | FAPEFDNHC | 1.19% |
| SEQ ID NO: 780 | REFAISGGS | 1.19% |
| SEQ ID NO: 781 | AFKTFGHVN | 1.18% |
| SEQ ID NO: 782 | ASSRLAEVI | 1.18% |
| SEQ ID NO: 783 | RMSAEKERS | 1.17% |
| SEQ ID NO: 784 | TMGLGVNAI | 1.17% |
| SEQ ID NO: 785 | PADWDREHG | 1.17% |
| SEQ ID NO: 786 | PELRPCAPR | 1.17% |
| SEQ ID NO: 787 | PDTQSAFWI | 1.17% |
| SEQ ID NO: 788 | KFKWLVRMT | 1.16% |
| SEQ ID NO: 789 | MTNRCAKYK | 1.15% |
| SEQ ID NO: 790 | AQPRTCQII | 1.13% |
| SEQ ID NO: 791 | IVVFGQMGY | 1.13% |
| SEQ ID NO: 792 | KVVEMFRWY | 1.12% |
| SEQ ID NO: 793 | CQTHWEKGD | 1.12% |
| SEQ ID NO: 794 | SGTEHVALH | 1.12% |
| SEQ ID NO: 795 | LCDLIMTDE | 1.11% |
| SEQ ID NO: 796 | PTLDRWEYH | 1.10% |
| SEQ ID NO: 797 | QLPIYIQKS | 1.08% |
| SEQ ID NO: 798 | HLAWIVNED | 1.00% |
| SEQ ID NO: 799 | HYFKWLFVE | 0.98% |
| SEQ ID NO: 800 | KFTWKKYFA | 0.97% |
| SEQ ID NO: 801 | HTWPPLFYW | 0.94% |
| SEQ ID NO: 802 | TWMPKVEYK | 0.91% |
| SEQ ID NO: 803 | ALTAVHAGT | 0.90% |
| SEQ ID NO: 804 | TLNSWHPEC | 0.90% |
| SEQ ID NO: 805 | AHMSYCLSR | 0.89% |
| SEQ ID NO: 806 | TDYPWVLHG | 0.89% |
| SEQ ID NO: 807 | WFSIIMSKL | 0.89% |
| SEQ ID NO: 808 | SVKVKNGFT | 0.89% |
| SEQ ID NO: 809 | NHIFKKVTW | 0.88% |
| SEQ ID NO: 810 | KPPACIGGK | 0.88% |
| SEQ ID NO: 811 | PPDARQSLR | 0.88% |
| SEQ ID NO: 812 | RQTGTGNME | 0.88% |
| SEQ ID NO: 813 | EQFADQAPR | 0.88% |
| SEQ ID NO: 814 | ISISAQDID | 0.88% |
| SEQ ID NO: 815 | KFTLQQMAS | 0.88% |
| SEQ ID NO: 816 | SFSGTHLGW | 0.88% |
| SEQ ID NO: 817 | NHSDTDTAQ | 0.86% |
| SEQ ID NO: 818 | AFCYSYYQN | 0.80% |
| SEQ ID NO: 819 | EGKITKAAV | 0.80% |
| SEQ ID NO: 820 | VQCLWWHKE | 0.77% |
| SEQ ID NO: 821 | QNLMYCSSV | 0.72% |
| SEQ ID NO: 822 | RRWLNPAYN | 0.69% |
| SEQ ID NO: 823 | PWFYKDWPH | 0.66% |
| SEQ ID NO: 824 | QALWLESDN | 0.62% |
| SEQ ID NO: 825 | TLNLRHPEC | 0.62% |
| SEQ ID NO: 826 | APEFRNRLT | 0.60% |
| SEQ ID NO: 827 | DVMRMGHYL | 0.60% |
| SEQ ID NO: 828 | ERRAHNHYA | 0.60% |
| SEQ ID NO: 829 | TSHKMMVLS | 0.59% |
| SEQ ID NO: 830 | EVDRNTNWC | 0.59% |
| SEQ ID NO: 831 | MALPLRVLS | 0.59% |
| SEQ ID NO: 832 | PCKSTRSIL | 0.59% |
| SEQ ID NO: 833 | TTDGMMGDH | 0.59% |
| SEQ ID NO: 834 | YPVHLMPPL | 0.59% |
| SEQ ID NO: 835 | ECWMAVQSC | 0.59% |
| SEQ ID NO: 836 | ERTACKTKS | 0.59% |
| SEQ ID NO: 837 | GTARHNMQL | 0.59% |
| SEQ ID NO: 838 | GTVDAQDRE | 0.59% |
| SEQ ID NO: 839 | RQIGTGNTE | 0.59% |
| SEQ ID NO: 840 | SPDGRGLCG | 0.59% |
| SEQ ID NO: 841 | TLNSRHLEC | 0.59% |
| SEQ ID NO: 842 | VLTDGACAA | 0.59% |
| SEQ ID NO: 843 | AHMYYCLSR | 0.58% |
| SEQ ID NO: 844 | AMLMSDGGG | 0.58% |
| SEQ ID NO: 845 | AQGFKPSGG | 0.58% |
| SEQ ID NO: 846 | ATGTYNLEE | 0.58% |
| SEQ ID NO: 847 | AYAASKNAM | 0.58% |
| SEQ ID NO: 848 | CAWYMKQGN | 0.58% |
| SEQ ID NO: 849 | CCRQIWIQY | 0.58% |
| SEQ ID NO: 850 | CMKTFPMNA | 0.58% |
| SEQ ID NO: 851 | DRINLMWKD | 0.58% |
| SEQ ID NO: 852 | DRVDLMWKD | 0.58% |
| SEQ ID NO: 853 | DSATFESAD | 0.58% |
| SEQ ID NO: 854 | EFEMRDSGY | 0.58% |
| SEQ ID NO: 855 | EKYQIHRDR | 0.58% |
| SEQ ID NO: 856 | HNQRTYPAL | 0.58% |
| SEQ ID NO: 857 | KMTNSSENS | 0.58% |
| SEQ ID NO: 858 | KQPTFPMLA | 0.58% |
| SEQ ID NO: 859 | LMRWYNRCW | 0.58% |
| SEQ ID NO: 860 | LRCTVMDPG | 0.58% |
| SEQ ID NO: 861 | LYQKDQRFG | 0.58% |
| SEQ ID NO: 862 | MHVNRRIDQ | 0.58% |
| SEQ ID NO: 863 | NCKSDQIAM | 0.58% |
| SEQ ID NO: 864 | NNHRLNDAN | 0.58% |
| SEQ ID NO: 865 | QPKVHGNYL | 0.58% |
| SEQ ID NO: 866 | QVEDRYWSY | 0.58% |
| SEQ ID NO: 867 | RRGYVNIWE | 0.58% |
| SEQ ID NO: 868 | SPCPTWDGL | 0.58% |
| SEQ ID NO: 869 | SRLLVHECP | 0.58% |
| SEQ ID NO: 870 | SSMQCVMRP | 0.58% |
| SEQ ID NO: 871 | SVTYHEWHL | 0.58% |
| SEQ ID NO: 872 | TCICSHTYP | 0.58% |
| SEQ ID NO: 873 | TGMAVSEQS | 0.58% |
| SEQ ID NO: 874 | TIEHGAWKC | 0.58% |
| SEQ ID NO: 875 | TPLTKWLPI | 0.58% |
| SEQ ID NO: 876 | VSIPNLGWP | 0.58% |
| SEQ ID NO: 877 | YCHLEDRIT | 0.58% |
| SEQ ID NO: 878 | EWMVIMAKN | 0.40% |
| SEQ ID NO: 879 | RVAIMFTGT | 0.35% |
| SEQ ID NO: 880 | IPDIMRRCP | 0.33% |
| SEQ ID NO: 881 | ERTACKMRS | 0.33% |
| SEQ ID NO: 882 | YTTVEGRIT | 0.33% |
| SEQ ID NO: 883 | DLEFCVMER | 0.32% |
| SEQ ID NO: 884 | YAQRTCWSE | 0.31% |
| SEQ ID NO: 885 | DRVNLTWKD | 0.31% |
| SEQ ID NO: 886 | DSTPNLLDP | 0.31% |
| SEQ ID NO: 887 | EPLYSWVGV | 0.31% |
| SEQ ID NO: 888 | KQLFMKFWS | 0.31% |
| SEQ ID NO: 889 | FVGLMSQCG | 0.31% |
| SEQ ID NO: 890 | KPCSSMSIQ | 0.31% |
| SEQ ID NO: 891 | SIYQKWVTS | 0.31% |
| SEQ ID NO: 892 | TLSSRHPEC | 0.31% |
| SEQ ID NO: 893 | AQTNNTCS | 0.31% |
| SEQ ID NO: 894 | IPSHVHLGE | 0.31% |
| SEQ ID NO: 895 | MALPPHVLS | 0.31% |
| SEQ ID NO: 896 | MGCIGNCIL | 0.31% |
| SEQ ID NO: 897 | QTQHYGSTT | 0.31% |
| SEQ ID NO: 898 | RVDRNTNWC | 0.31% |
| SEQ ID NO: 899 | TYDTRSHCL | 0.31% |
| SEQ ID NO: 900 | ARVDHQCSG | 0.30% |
| SEQ ID NO: 901 | NGSHYNAMR | 0.30% |
| SEQ ID NO: 902 | QTQLYQNDE | 0.30% |
| SEQ ID NO: 903 | SPYGRGLCG | 0.30% |
| SEQ ID NO: 904 | TYSLAMSLG | 0.30% |
| SEQ ID NO: 905 | WVGNANRPA | 0.30% |
| SEQ ID NO: 906 | AHVDHQCGG | 0.30% |
| SEQ ID NO: 907 | APEFNVARR | 0.30% |
| SEQ ID NO: 908 | CCQQIWVQY | 0.30% |
| SEQ ID NO: 909 | CITARSTAA | 0.30% |
| SEQ ID NO: 910 | CTIARSTAA | 0.30% |
| SEQ ID NO: 911 | DVIRTTHSG | 0.30% |
| SEQ ID NO: 912 | GSQQTTPSC | 0.30% |
| SEQ ID NO: 913 | GYATFPDSD | 0.30% |
| SEQ ID NO: 914 | ISSTMVEHG | 0.30% |
| SEQ ID NO: 915 | KLLFMKFWN | 0.30% |
| SEQ ID NO: 916 | KVDRNTYWC | 0.30% |
| SEQ ID NO: 917 | LFRAYAWSQ | 0.30% |
| SEQ ID NO: 918 | LITRDTGLA | 0.30% |
| SEQ ID NO: 919 | LSECSDRTN | 0.30% |
| SEQ ID NO: 920 | NCKPNQIAM | 0.30% |
| SEQ ID NO: 921 | PAVPADEFF | 0.30% |
| SEQ ID NO: 922 | QATDMHRWQ | 0.30% |
| SEQ ID NO: 923 | STARHNTQL | 0.30% |
| SEQ ID NO: 924 | THMNYCLSR | 0.30% |
| SEQ ID NO: 925 | TSHRMMVFS | 0.30% |
| SEQ ID NO: 926 | AMTRCMAGH | 0.30% |
| SEQ ID NO: 927 | CPEHNLVGV | 0.30% |
| SEQ ID NO: 928 | CPEHSQVGV | 0.30% |
| SEQ ID NO: 929 | DRANLMWKD | 0.30% |
| SEQ ID NO: 930 | DRVNLMWKG | 0.30% |
| SEQ ID NO: 931 | DWGYCHSKA | 0.30% |
| SEQ ID NO: 932 | EFVLGYQGI | 0.30% |
| SEQ ID NO: 933 | EKYQIHWDH | 0.30% |
| SEQ ID NO: 934 | EQFVDRAPR | 0.30% |
| SEQ ID NO: 935 | ERQAHSHYA | 0.30% |
| SEQ ID NO: 936 | FIYQKWVTN | 0.30% |
| SEQ ID NO: 937 | GQFRSGAHR | 0.30% |
| SEQ ID NO: 938 | HTNEQYLQH | 0.30% |
| SEQ ID NO: 939 | HVNTSCKQS | 0.30% |
| SEQ ID NO: 940 | KYSPIQEHN | 0.30% |
| SEQ ID NO: 941 | LCLQGAKMD | 0.30% |
| SEQ ID NO: 942 | LMSITSAVE | 0.30% |
| SEQ ID NO: 943 | LTHNIPFPK | 0.30% |
| SEQ ID NO: 944 | MALPLHALS | 0.30% |
| SEQ ID NO: 945 | MCQSVEKEY | 0.30% |
| SEQ ID NO: 946 | NGNRYNAMR | 0.30% |
| SEQ ID NO: 947 | NHRSREIDI | 0.30% |
| SEQ ID NO: 948 | NIVRSRSIT | 0.30% |
| SEQ ID NO: 949 | NKTCYLTCP | 0.30% |
| SEQ ID NO: 950 | PQARVDMEF | 0.30% |
| SEQ ID NO: 951 | QAAGMHRWQ | 0.30% |
| SEQ ID NO: 952 | REFVISGGN | 0.30% |
| SEQ ID NO: 953 | SMREYQKHI | 0.30% |
| SEQ ID NO: 954 | SPHSCQHMG | 0.30% |
| SEQ ID NO: 955 | SRLSVHECP | 0.30% |
| SEQ ID NO: 956 | STTRHNMQL | 0.30% |
| SEQ ID NO: 957 | TFHFSILLT | 0.30% |
| SEQ ID NO: 958 | TPQPMNVAA | 0.30% |
| SEQ ID NO: 959 | TYIQHPTNA | 0.30% |
| SEQ ID NO: 960 | VAGRFGSNT | 0.30% |
| SEQ ID NO: 961 | VSMPSLGWP | 0.30% |
| SEQ ID NO: 962 | YDNHQKARI | 0.30% |
| SEQ ID NO: 963 | AHMIYCLSR | 0.29% |
| SEQ ID NO: 964 | AHMNYCLSH | 0.29% |
| SEQ ID NO: 965 | AQWIQQKHG | 0.29% |
| SEQ ID NO: 966 | ARTLREGWG | 0.29% |
| SEQ ID NO: 967 | ASKLEVQEQ | 0.29% |
| SEQ ID NO: 968 | AVEHGAWKC | 0.29% |
| SEQ ID NO: 969 | AVETGESAV | 0.29% |
| SEQ ID NO: 970 | CCRQIWVQH | 0.29% |
| SEQ ID NO: 971 | CGGQETGLA | 0.29% |
| SEQ ID NO: 972 | CNSWCTGSL | 0.29% |
| SEQ ID NO: 973 | DCRHTTLYV | 0.29% |
| SEQ ID NO: 974 | DESTCVAHG | 0.29% |
| SEQ ID NO: 975 | DGMHRELHS | 0.29% |
| SEQ ID NO: 976 | DKYGLGIFH | 0.29% |
| SEQ ID NO: 977 | DRVYLMWKD | 0.29% |
| SEQ ID NO: 978 | DTDYKPHFA | 0.29% |
| SEQ ID NO: 979 | DVHDGQLCY | 0.29% |
| SEQ ID NO: 980 | DVYDSQLCY | 0.29% |
| SEQ ID NO: 981 | DYAPTNTMM | 0.29% |
| SEQ ID NO: 982 | DYSQVNCER | 0.29% |
| SEQ ID NO: 983 | DYSQVNREK | 0.29% |
| SEQ ID NO: 984 | EEYYTWECH | 0.29% |
| SEQ ID NO: 985 | ETHHEYRDD | 0.29% |
| SEQ ID NO: 986 | EYACMLMTR | 0.29% |
| SEQ ID NO: 987 | FLRPKRHFS | 0.29% |
| SEQ ID NO: 988 | GCQLGFQIT | 0.29% |
| SEQ ID NO: 989 | GQFRRGAYR | 0.29% |
| SEQ ID NO: 990 | GSLSKDTPL | 0.29% |
| SEQ ID NO: 991 | GSQQTTLSR | 0.29% |
| SEQ ID NO: 992 | HCHQEDRIT | 0.29% |
| SEQ ID NO: 993 | HHECLYQIY | 0.29% |
| SEQ ID NO: 994 | HVDQHRIQA | 0.29% |
| SEQ ID NO: 995 | IADIGRKQW | 0.29% |
| SEQ ID NO: 996 | IAWPTDCSF | 0.29% |
| SEQ ID NO: 997 | IYCPCQDSD | 0.29% |
| SEQ ID NO: 998 | KAALFLKDC | 0.29% |
| SEQ ID NO: 999 | KAGMDQTAV | 0.29% |
| SEQ ID NO: 1000 | KCRMAVQSC | 0.29% |
| SEQ ID NO: 1001 | KPLASIGQH | 0.29% |
| SEQ ID NO: 1002 | KQLFMEFWN | 0.29% |
| SEQ ID NO: 1003 | KSLQEQGGA | 0.29% |
| SEQ ID NO: 1004 | KTQLSDHKH | 0.29% |
| SEQ ID NO: 1005 | LDHTNLMKL | 0.29% |
| SEQ ID NO: 1006 | LIQWYNRCW | 0.29% |
| SEQ ID NO: 1007 | LMKTSPCYW | 0.29% |
| SEQ ID NO: 1008 | LMLGYMEQD | 0.29% |
| SEQ ID NO: 1009 | LSECSDQAN | 0.29% |
| SEQ ID NO: 1010 | LVVIDPAHY | 0.29% |
| SEQ ID NO: 1011 | MALPLYVLS | 0.29% |
| SEQ ID NO: 1012 | MCQMSRRSI | 0.29% |
| SEQ ID NO: 1013 | MMLGYMERD | 0.29% |
| SEQ ID NO: 1014 | MTQVHQALV | 0.29% |
| SEQ ID NO: 1015 | NACPQHDFG | 0.29% |
| SEQ ID NO: 1016 | NALWMTKAP | 0.29% |
| SEQ ID NO: 1017 | NCRHTILYV | 0.29% |
| SEQ ID NO: 1018 | NDCTCRAIL | 0.29% |
| SEQ ID NO: 1019 | NFANHQPLP | 0.29% |
| SEQ ID NO: 1020 | NQRGLCKDE | 0.29% |
| SEQ ID NO: 1021 | PACLLRRML | 0.29% |
| SEQ ID NO: 1022 | PANPSFSYL | 0.29% |
| SEQ ID NO: 1023 | PCQQLMCFT | 0.29% |
| SEQ ID NO: 1024 | PDARQSLRH | 0.29% |
| SEQ ID NO: 1025 | PIYQKWVTN | 0.29% |
| SEQ ID NO: 1026 | PNGCRPFCC | 0.29% |
| SEQ ID NO: 1027 | PQHAGRAEH | 0.29% |
| SEQ ID NO: 1028 | QCFLWNDYQ | 0.29% |
| SEQ ID NO: 1029 | QHLMTLSHE | 0.29% |
| SEQ ID NO: 1030 | QPLSRNGAR | 0.29% |
| SEQ ID NO: 1031 | QPQLRNGAR | 0.29% |
| SEQ ID NO: 1032 | QRQSQEWFH | 0.29% |
| SEQ ID NO: 1033 | QTGVMAAIG | 0.29% |
| SEQ ID NO: 1034 | QVVIIECKN | 0.29% |
| SEQ ID NO: 1035 | RDRRVRDEC | 0.29% |
| SEQ ID NO: 1036 | RLETTRYIW | 0.29% |
| SEQ ID NO: 1037 | RMARVSVHG | 0.29% |
| SEQ ID NO: 1038 | RQHHCLPVI | 0.29% |
| SEQ ID NO: 1039 | RQVGTGNME | 0.29% |
| SEQ ID NO: 1040 | RSTRFTGIA | 0.29% |
| SEQ ID NO: 1041 | RTNEQYPQH | 0.29% |
| SEQ ID NO: 1042 | RTRISQMSG | 0.29% |
| SEQ ID NO: 1043 | RTRTSQMFG | 0.29% |
| SEQ ID NO: 1044 | RVNGSTCGH | 0.29% |
| SEQ ID NO: 1045 | SATTSGTAF | 0.29% |
| SEQ ID NO: 1046 | SATYHEWHP | 0.29% |
| SEQ ID NO: 1047 | SEAKYDTAT | 0.29% |
| SEQ ID NO: 1048 | SKRWYGHSA | 0.29% |
| SEQ ID NO: 1049 | SMQEYQKHV | 0.29% |
| SEQ ID NO: 1050 | SRCTVVDPG | 0.29% |
| SEQ ID NO: 1051 | SSWMQMQGP | 0.29% |
| SEQ ID NO: 1052 | TAMVSECID | 0.29% |
| SEQ ID NO: 1053 | TEFHMMIQT | 0.29% |
| SEQ ID NO: 1054 | TGRWLVYQA | 0.29% |
| SEQ ID NO: 1055 | TLNSRHPEY | 0.29% |
| SEQ ID NO: 1056 | TSEPHCSFA | 0.29% |
| SEQ ID NO: 1057 | TVDGMMGDH | 0.29% |
| SEQ ID NO: 1058 | TVPKWDNIS | 0.29% |
| SEQ ID NO: 1059 | TYHCYMSWM | 0.29% |
| SEQ ID NO: 1060 | TYIQCPTNA | 0.29% |
| SEQ ID NO: 1061 | TYIQYPANA | 0.29% |
| SEQ ID NO: 1062 | VHGQTDLSL | 0.29% |
| SEQ ID NO: 1063 | VPVPFLSLR | 0.29% |
| SEQ ID NO: 1064 | VSFQRMNNM | 0.29% |
| SEQ ID NO: 1065 | VSTPNLGWP | 0.29% |
| SEQ ID NO: 1066 | VSVQGRAQL | 0.29% |
| SEQ ID NO: 1067 | VTEVLNQDV | 0.29% |
| SEQ ID NO: 1068 | YATHMQHLE | 0.29% |
| SEQ ID NO: 1069 | YCHQEDRII | 0.29% |
| SEQ ID NO: 1070 | YETDSEGYD | 0.29% |
| SEQ ID NO: 1071 | YPAHMMPPL | 0.29% |
| SEQ ID NO: 1072 | YWCPSQRWR | 0.29% |
| SEQ ID NO: 1073 | QLKYAVGAN | 0.23% |
| SEQ ID NO: 1074 | MNHQCMLWD | 0.07% |
| SEQ ID NO: 1075 | RHQRVMPTY | 0.05% |
| SEQ ID NO: 1076 | NSGIATADH | 0.05% |
| SEQ ID NO: 1077 | ACHTGQSGM | 0.04% |
| SEQ ID NO: 1078 | SHWFFHFDL | 0.03% |
| SEQ ID NO: 1079 | DVTQTRYCY | 0.03% |
| SEQ ID NO: 1080 | FSEQNVSAA | 0.03% |
| SEQ ID NO: 1081 | GPLYSWVGI | 0.03% |
| SEQ ID NO: 1082 | NAGMKGVDV | 0.03% |
| SEQ ID NO: 1083 | CTEAGMTFG | 0.02% |
| SEQ ID NO: 1084 | PMNDMQFVP | 0.02% |
| SEQ ID NO: 1085 | SGRRSGGRP | 0.02% |
| SEQ ID NO: 1086 | DVAQYGLGS | 0.02% |
| SEQ ID NO: 1087 | NPTDLLQHE | 0.02% |
| SEQ ID NO: 1088 | QAQCIAMQR | 0.02% |
| SEQ ID NO: 1089 | DGMHRELCS | 0.02% |
| SEQ ID NO: 1090 | DGTHRELYS | 0.02% |
| SEQ ID NO: 1091 | KWCQFGMVA | 0.02% |
| SEQ ID NO: 1092 | LTHNTPFPE | 0.02% |
| SEQ ID NO: 1093 | PMPTADVLH | 0.02% |
| SEQ ID NO: 1094 | QGEEVWWEA | 0.02% |
| SEQ ID NO: 1095 | RMVTMERDR | 0.02% |
| SEQ ID NO: 1096 | RVNGSTCWH | 0.02% |
| SEQ ID NO: 1097 | RWYQFGMVA | 0.02% |
| SEQ ID NO: 1098 | TFLEYQALG | 0.02% |
| SEQ ID NO: 1099 | TTGVCSFAQ | 0.02% |
| SEQ ID NO: 1100 | YHKQMAVWW | 0.02% |
| SEQ ID NO: 1101 | AVSVACLNT | 0.01% |
| SEQ ID NO: 1102 | CDSPSLLQS | 0.01% |
| SEQ ID NO: 1103 | CMYLPALQY | 0.01% |
| SEQ ID NO: 1104 | CSSWANGCD | 0.01% |
| SEQ ID NO: 1105 | EDCPKVFTI | 0.01% |
| SEQ ID NO: 1106 | EFALGCQGI | 0.01% |
| SEQ ID NO: 1107 | ERGEMRATG | 0.01% |
| SEQ ID NO: 1108 | ERGGMKATG | 0.01% |
| SEQ ID NO: 1109 | GRVHGNVCI | 0.01% |
| SEQ ID NO: 1110 | HVWSLEWHL | 0.01% |
| SEQ ID NO: 1111 | IPGHVHFGE | 0.01% |
| SEQ ID NO: 1112 | IWGVYVHYF | 0.01% |
| SEQ ID NO: 1113 | NHDEKLWAP | 0.01% |
| SEQ ID NO: 1114 | NVWSLAVRD | 0.01% |

CNS (cortex forebrain, cortex occipital, cortex temporal, thalamus, hypothalamus, substantia nigra, hippocampus DG, hippocampus CA1, hippocampus CA3, cerebellum), liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive CNS tropism. The results are shown in FIG. 18B which shows the positional frequency of each amino acid in the 581 to 589 region of AAV5 VP1 in CNS tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in CNS over the frequency of that given amino acid residue occurring at the specified position in variants identified in all other harvested tissues was analyzed to identify a set of sequence rules for capsids that preferentially target CNS tissue. With reference to TABLE 9 below, and SEQ ID NO: 2 (AAV5 VP1), the following amino acids can, thus, be independently mutated, in any combination, at any one or more positions Xaa1-Xaa9, to provide a VP1 capsid with enhanced CNS tropism as compared wildtype AAV5 VP1 capsid (SEQ ID NO: 1), where CNS tropism here refers to properties that are preferred for CNS transduction over properties that are preferred for transduction of all other harvested tissues.

TABLE 9
CNS Tropism Rules

Xaa1 is selected from A, C, K, M, Q, R, T, or W
Xaa1 is selected from K, Q, R, or W
Xaa1 is K
Xaa2 is selected from F, I, K, R, T, or W
Xaa2 is selected from F, I, R or T
Xaa2 is R
Xaa3 is selected from A, H, N, R, or W
Xaa3 is selected from A, R, or W
Xaa3 is R
Xaa4 is selected from E, G, I, M, Q, or R
Xaa4 is selected from E, M, or R
Xaa4 is R
Xaa5 is selected from C, G, K, I, M, or R
Xaa5 is selected from K, I, or R
Xaa5 is I
Xaa6 is selected from I, K, L, P, Q, R, Y
Xaa6 is selected from K, R, or Y
Xaa6 is R
Xaa7 is selected from D, I, K, R, V, or W
Xaa7 is selected from I, R, or V
Xaa7 is V
Xaa8 is selected from C, G, H, K, L, or V
Xaa8 is selected from H, K, or V
Xaa8 is H
Xaa9 is selected from I, K, L, R, or V
Xaa9 is selected from I, K, or R
Xaa9 is R

TABLE 10 below provides sequences of the 581 to 589 region for variant AAV VP1 capsid polypeptides that were found in CNS tissue and comport to one or more of the rules provided in TABLE 9. The present disclosure, thus, provides for rAAVs composed of engineered AAV5 VP1 capsid polypeptides having a SEQ ID NO: 2, wherein the Xaa1 to Xaa9 region is any one of SEQ ID NO: 7118-SEQ ID NO: 8117, as disclosed in TABLE 10. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 10
Sequences of the 581 to 589 Region in AAV5 VP1
Capsid Polypeptide that Drive CNS Tissue
Tropism

| SEQ | |
|---|---|
| ID | 581-589 |
| NO | Sequence |
| 7118 | KTTFDVACY |
| 7119 | KDGYREAWM |
| 7120 | KTSNVMDQM |
| 7121 | KTLHMLESK |
| 7122 | KSHDYHGRA |
| 7123 | KSEWTCCPQ |
| 7124 | KMHGNERCF |
| 7125 | KQTVFVDQE |
| 7126 | KVAHEMMSA |
| 7127 | KMNTWDQAR |
| 7128 | KFNTKDQLM |
| 7129 | KCQSKECGG |
| 7130 | KGLITENNY |
| 7131 | KVEWRWGSA |
| 7132 | KTFQYMGPT |
| 7133 | KCGLTPGVE |
| 7134 | KQRIAYEWQ |
| 7135 | KGQRRDSVS |
| 7136 | KQCSLAPTI |
| 7137 | KEWCVQQQC |
| 7138 | KKDPLHLGD |
| 7139 | KQTKSMVYM |
| 7140 | KISGLNDAS |
| 7141 | KPDILKTKT |
| 7142 | KWSAYFLIE |
| 7143 | KGESQCTSQ |
| 7144 | KQEYIMRQR |
| 7145 | KLDPRQADQ |
| 7146 | KIYMQCDQE |
| 7147 | KWAASHKHD |
| 7148 | KENKIMQAS |
| 7149 | KVAGGHETT |
| 7150 | KTDPIAEYC |
| 7151 | KPDHAMAQQ |
| 7152 | KSDKVWYVL |
| 7153 | KIEAEEMHA |
| 7154 | KKTECEMIV |
| 7155 | KGTPNWWGP |
| 7156 | KPAGKNTCW |
| 7157 | KSVHYETCE |
| 7158 | KCNVITYHG |
| 7159 | KQEQFHTGS |
| 7160 | KTMADMYNR |
| 7161 | KTVETYAAH |
| 7162 | KSWVWVDTE |
| 7163 | KAIWQGTEN |
| 7164 | KPLTYTVGS |
| 7165 | KYASMDIYS |
| 7166 | KCTMITPCF |
| 7167 | KEAYTDMSC |
| 7168 | KHMPTLQWQ |
| 7169 | KIEYGHWIH |
| 7170 | KYSPFQEHN |
| 7171 | KHTLAEMYG |
| 7172 | KIWAGVNLG |
| 7173 | KCDDRNTQH |
| 7174 | KSIMMSQLD |
| 7175 | KKIFCSWDY |
| 7176 | KQAGYDFFH |
| 7177 | KSDAQRNVA |
| 7178 | KPPSWPMSL |
| 7179 | KAHDITWNL |
| 7180 | KQATNNNGW |
| 7181 | KDPDCYDWP |
| 7182 | KHTEYCSHE |
| 7183 | KWYHYNEMT |
| 7184 | KGQMISETR |
| 7185 | KHEPMCVQL |
| 7186 | KVDTVRNPK |
| 7187 | KRVQQMETM |
| 7188 | KIIFELGMD |
| 7189 | KSGWGAPYE |
| 7190 | KIQMAVSLD |
| 7191 | KVPSQLVFE |
| 7192 | KSPDMTLLL |
| 7193 | KSMTCVYYP |
| 7194 | KPDKPNDYA |
| 7195 | KFICTTIAC |
| 7196 | KPAYEHRGF |
| 7197 | KNNTEDGGR |
| 7198 | KATREYDIP |
| 7199 | KCETKKRFN |
| 7200 | KTEKWASHE |
| 7201 | KATVEGSWR |
| 7202 | KRRDGTRSY |
| 7203 | KALHYAMIK |
| 7204 | KWEVMSAAY |
| 7205 | KALYLHTIG |
| 7206 | KLNPFSTGT |
| 7207 | KVEIINWHP |
| 7208 | KNDPIDNFG |
| 7209 | KTFTKEVTP |
| 7210 | KCGNEGRMS |
| 7211 | KNETFIAST |
| 7212 | KDNQSCEWN |
| 7213 | KAQVVPVEC |
| 7214 | KMMAFEKQC |
| 7215 | KWQEMPTFQ |
| 7216 | KYCPPQTPN |
| 7217 | KCAMFNAGE |
| 7218 | KSTIPQTYP |
| 7219 | KFTEALSCG |
| 7220 | KGEIEGGAK |
| 7221 | KQLTFPMLA |
| 7222 | KEFVGFQAM |
| 7223 | KHDGFSVVG |
| 7224 | KATRDPKFP |
| 7225 | KRWLHMYQG |
| 7226 | KHLDMTSCH |
| 7227 | KDEQFARCK |
| 7228 | KTLPCSNSE |
| 7229 | KYMQENVLS |
| 7230 | KSAPRENFN |
| 7231 | KQRMTRQGE |
| 7232 | KARQDDVNT |
| 7233 | KCGVEHEHR |
| 7234 | KSELFMKLH |
| 7235 | KNELENVIN |
| 7236 | KYWCKNEWD |
| 7237 | KASLGACVE |
| 7238 | KSCNDGQQN |
| 7239 | KVTPILEEV |
| 7240 | KQEWHVHTE |
| 7241 | KPKCDEQKS |
| 7242 | KWHGGPGVV |
| 7243 | KGNSVTHVQ |
| 7244 | KWDDMHTQM |
| 7245 | KQAHLEVAW |
| 7246 | KVNACGNLT |
| 7247 | KAIPQGSSC |
| 7248 | KINCRMDGC |
| 7249 | KQRDWENFA |
| 7250 | KMCWWPESM |
| 7251 | KQNLMCCHN |
| 7252 | KISPWDGFY |
| 7253 | KGMQMWNCE |
| 7254 | KAILNAMME |
| 7255 | KIDAHSNME |
| 7256 | KAPWAPMDS |
| 7257 | KRAIVGYAL |
| 7258 | KSGMIQEET |
| 7259 | KSYQMIMTW |
| 7260 | KFELHGTVG |
| 7261 | KSGTCGYGQ |
| 7262 | KSLQCASRH |
| 7263 | KTGTTMEPM |
| 7264 | KTDYSRLSD |
| 7265 | KHYKWGHCQ |
| 7266 | KLYGLNHSC |
| 7267 | KLKESMESA |
| 7268 | KAGYQEGSC |
| 7269 | KCWDCHEAG |
| 7270 | KPVQCACQN |
| 7271 | KDYWAGYQM |
| 7272 | KSEHHWINA |
| 7273 | KGGPWAYNC |
| 7274 | KYAYSLDCA |
| 7275 | KLWIDQRGH |
| 7276 | KLWIDQRGY |
| 7277 | QRVTKMHFE |
| 7278 | ERTACKMKS |
| 7279 | ERGEMKATG |
| 7280 | ARGTCNYCY |
| 7281 | QRNEMSASR |
| 7282 | FRDVCMHDV |
| 7283 | ERMMWEEYP |
| 7284 | HRMSYKWGW |
| 7285 | IRVADDMTC |
| 7286 | MRSICEHAM |
| 7287 | NRGIWLINF |
| 7288 | NRQFWYLNT |
| 7289 | PRLLRPNRV |
| 7290 | TRRVATHMP |
| 7291 | VRHSRNNTV |
| 7292 | SRWFQIGTE |
| 7293 | TRQWMCVHC |
| 7294 | HRVHSEKTD |
| 7295 | GRHTAFATG |
| 7296 | LRSRVIKDH |
| 7297 | IRMIGKACN |
| 7298 | NRLDQITRR |
| 7299 | SRSWFDSCG |
| 7300 | VRCQQHQGN |
| 7301 | YRSWMCWDI |
| 7302 | CRNTQYEQH |
| 7303 | ARKNCDHLN |
| 7304 | ARCQNHWSH |
| 7305 | MRQRTYICQ |
| 7306 | SRCTVMDPG |
| 7307 | PRNLVAMYW |
| 7308 | TRMQAVDHE |
| 7309 | FRAVYYYHS |
| 7310 | RREAMDDSP |
| 7311 | GRQSINEIA |
| 7312 | TRHPTIAMH |
| 7313 | YRLHVWMKV |
| 7314 | DRREKQLMK |
| 7315 | ERKAMRWSL |
| 7316 | LRMQKEGQE |
| 7317 | IRPQYAKMD |
| 7318 | SRTICVHGN |
| 7319 | NRCGVSKCC |
| 7320 | CRCNDAWNM |
| 7321 | FRKQGHGEK |
| 7322 | YRMEYGTCE |
| 7323 | SRPFNYFQR |
| 7324 | QRGPLQHQQ |
| 7325 | MRQCNYDFL |
| 7326 | MRQCNYDFW |
| 7327 | MRQCNYGFW |
| 7328 | SRTRMYMDH |
| 7329 | ERTLTKAGS |
| 7330 | WRLAALYQS |
| 7331 | CRCCPVMKD |
| 7332 | YRDRCWNRI |
| 7333 | LRNMRGDWD |
| 7334 | CRKCEISAR |
| 7335 | ARTQFHSDE |
| 7336 | ERSMKDADE |
| 7337 | YRRWQSNTT |
| 7338 | HRMNNHAWF |
| 7339 | QRTQCNFSI |
| 7340 | TRILNEFFC |
| 7341 | ARLEIEYMG |
| 7342 | WRFCDMRMS |
| 7343 | DRLYCKLAH |
| 7344 | ERKTTADWK |
| 7345 | GRSMVLHFV |
| 7346 | TRHDNGPSD |
| 7347 | PRAPIRPPQ |
| 7348 | MRRKTPEDD |
| 7349 | ERFRVQEFQ |
| 7350 | ERYYPISSF |
| 7351 | GRAHGNVCI |
| 7352 | ARSGINGHE |
| 7353 | ARVALVKPG |
| 7354 | SRYVNNGTV |
| 7355 | ARAYNSYQA |
| 7356 | PRFKACHDP |
| 7357 | QRQGQLHYW |
| 7358 | SRCTIDVPI |
| 7359 | DRTMCKVQG |
| 7360 | LRVSQSFSN |
| 7361 | NRTNSPYVH |
| 7362 | CRGSLSNAN |
| 7363 | VRQSVPHWA |
| 7364 | HRQESPNNK |
| 7365 | TRNIGGVFQ |
| 7366 | RRWMNAKAT |
| 7367 | YRAHSAGYC |
| 7368 | YRAHSAGYY |
| 7369 | PPRTVARFK |
| 7370 | MTRSCQTEG |
| 7371 | SMRNSKGSG |
| 7372 | GGRSLHAYL |
| 7373 | GTRIYGKVS |
| 7374 | EVRSTRKNH |
| 7375 | GLRHGFYAV |
| 7376 | LARTTTNSE |
| 7377 | VVREQFGIH |
| 7378 | ANRADWQHL |
| 7379 | WCRTPQREN |
| 7380 | WKRTAGVHF |
| 7381 | GYRNYFIWP |
| 7382 | TARNFGDPP |
| 7383 | EARISGMHD |
| 7384 | MARLYHPQM |
| 7385 | SNRYVSLFI |
| 7386 | PWRWHKSAC |
| 7387 | TLRAEMSFH |
| 7388 | EQRHISRAP |
| 7389 | TKRIKCCWE |
| 7390 | MKRAPTLDY |
| 7391 | ENRIAVWFN |
| 7392 | AERDHKTIY |
| 7393 | ELRMSFTKV |
| 7394 | CVRIGMREP |
| 7395 | TARSTSDKC |
| 7396 | GKRVEDTSC |
| 7397 | NQRGFCKDE |
| 7398 | TPRCIRADG |
| 7399 | DQRMLLKEL |
| 7400 | SNRYYSRIF |
| 7401 | EDRMGLPHN |
| 7402 | EGRTISYGN |
| 7403 | ASRSCMTQE |
| 7404 | QSRQMMLLH |
| 7405 | MTRVSMTWR |
| 7406 | TFRHWEGHH |
| 7407 | VARQMQENQ |
| 7408 | INRPMGHGM |
| 7409 | CIRFMMREG |
| 7410 | FFRWRWGLG |
| 7411 | QGRWACWYS |
| 7412 | HHRVNYEMY |
| 7413 | SVRTCHQDE |
| 7414 | DDRVKMFHY |
| 7415 | GMRYSEWHW |
| 7416 | ETREHHLLV |
| 7417 | MFRQFYIQG |
| 7418 | TTRQISNID |
| 7419 | DCRHTILYV |
| 7420 | AIRQFMMHL |
| 7421 | LMRWSKDSN |
| 7422 | LMRGSKDSN |
| 7423 | LMRWSKDSS |
| 7424 | LMRWSRDSN |
| 7425 | ASRQFETCW |
| 7426 | SDRMVSISA |
| 7427 | NTRIVCDLS |
| 7428 | EFRCRLIGS |
| 7429 | DCRCYDCHD |
| 7430 | TMRDSDYNW |
| 7431 | DVRACMEDL |
| 7432 | WCRCKRFIE |
| 7433 | WIRSAQRPG |
| 7434 | MGRPHAWWG |
| 7435 | NFRMQTGCH |
| 7436 | SMRDLIAEA |
| 7437 | TARDREHAM |
| 7438 | CSRNRCWSG |
| 7439 | DYREKYKCQ |
| 7440 | VCRQKTDSW |
| 7441 | YSRMEENDR |
| 7442 | PIRYVKPAR |
| 7443 | QPRVYEGFY |
| 7444 | MDRMMYMLG |
| 7445 | VNRGDLSLF |
| 7446 | HTRKEGFNS |
| 7447 | LFRTIPIWQ |
| 7448 | APRREEQNC |
| 7449 | NTRQINGVD |
| 7450 | DARHKWCLE |
| 7451 | AERQACTMG |
| 7452 | ISRMLRQSS |
| 7453 | ETRVREDAV |
| 7454 | AMRAGLENS |
| 7455 | TCRWFAHYS |
| 7456 | QVRFEQNPA |
| 7457 | NERVYGWGM |
| 7458 | EKRLFDRTY |
| 7459 | VSRCGIDRS |
| 7460 | DARKTVNEW |
| 7461 | TIRYIGMSG |
| 7462 | FFRICGSYI |
| 7463 | NTRSDLRAG |
| 7464 | ANRMIWYPY |
| 7465 | AQRCFHRWG |
| 7466 | TCRYEELAD |
| 7467 | STRTTDWNM |
| 7468 | DARKTYICY |
| 7469 | SSRGQDGVM |
| 7470 | RTREPFWWW |
| 7471 | IERWWVIDV |
| 7472 | ENRSPNNPA |
| 7473 | DWRYFCVSL |
| 7474 | NPRQGFVPV |
| 7475 | NSRLLIKHL |
| 7476 | HTRVVKSTG |
| 7477 | EIRQFVKGA |
| 7478 | QERSMNAWW |
| 7479 | CARLEGRGN |
| 7480 | FMRDVFAWD |
| 7481 | MLRTQIHCA |
| 7482 | QLRCEYQHT |
| 7483 | CCRMDNKKS |
| 7484 | YIRVGGNNE |
| 7485 | NVRNFFDHC |
| 7486 | ATRWVQMGQ |
| 7487 | MDRDIPHCR |
| 7488 | CYRRSDSFC |
| 7489 | EYSRCCHQE |
| 7490 | VHLRSHTWF |
| 7491 | QQLRCHCFS |
| 7492 | AAIRYDPGF |
| 7493 | MSNRIIDIQ |
| 7494 | VTQRPLHHS |
| 7495 | NQQRTYNNG |
| 7496 | SLGRIECDF |
| 7497 | SADRTAMFH |
| 7498 | NILRYLICD |
| 7499 | TFMRKICGM |
| 7500 | CWARSVAQN |
| 7501 | ELPRYYGQM |
| 7502 | EYSRMNKAS |
| 7503 | DKVRQGQKD |
| 7504 | EFNRYPHGN |
| 7505 | HEMRRDYKS |
| 7506 | QVYRMMMQP |
| 7507 | TVNRAMREE |
| 7508 | YKLRSSFRW |
| 7509 | FANRCPPDD |
| 7510 | NKIRDVAVI |
| 7511 | PVARKMSTC |
| 7512 | VIQRKFDSG |
| 7513 | ALDRERLIF |
| 7514 | AGSRMQWAM |
| 7515 | WSSREYWHK |
| 7516 | THARFKMKS |
| 7517 | NCARGPMEK |
| 7518 | LWSRFISMD |
| 7519 | IAMRCWGNE |
| 7520 | LVDRSGRTA |
| 7521 | SQSRTIIQE |
| 7522 | HEVRRYGQN |
| 7523 | NSTRNTSYY |
| 7524 | GPARSFEAC |
| 7525 | YCPRLVEAW |
| 7526 | VIGRTVDDP |
| 7527 | WFDRSWHIL |
| 7528 | CYIRNMVNL |
| 7529 | LIGRDSKVW |
| 7530 | QNIRLGVPT |
| 7531 | VICRMKKHS |
| 7532 | VEDRVFACC |
| 7533 | GHLRDHPLY |
| 7534 | FIFRKCDHW |
| 7535 | RKTRCFCNY |
| 7536 | TSERHSCMV |
| 7537 | SMSRHMMHQ |
| 7538 | DIGRFTAYY |
| 7539 | GVSRLPYHA |
| 7540 | QVDRTMWMH |
| 7541 | CILRKPSYH |
| 7542 | NNDRHQHLK |
| 7543 | TVDRKVEIE |
| 7544 | NSARYAWVY |
| 7545 | AITRHVADT |
| 7546 | TDKRAHVGA |
| 7547 | LLWRRVKRT |
| 7548 | YVIRKTWFN |
| 7549 | FAERANYCC |
| 7550 | SYMRHPNFD |
| 7551 | GGDRMATDR |
| 7552 | FQTRPSCSF |
| 7553 | MMVRREFAD |
| 7554 | AVMRNDQWL |
| 7555 | LQERDHHMQ |
| 7556 | LTPRSEGLS |
| 7557 | LVGRTYQHQ |
| 7558 | DATRAQTGE |
| 7559 | MMVRELMSS |
| 7560 | CGHRHSSMG |
| 7561 | HHLRNVHHC |
| 7562 | NSTRWYHTG |
| 7563 | NHMRKQSSH |
| 7564 | DDCRLNVRT |
| 7565 | MCARGAMCA |
| 7566 | SWGRIPHES |
| 7567 | QLKRNEQTS |
| 7568 | RNQRTYPAL |
| 7569 | SFFREDYSF |
| 7570 | DSHRCLKAD |
| 7571 | SAPRNEITR |
| 7572 | DIQRWEHEG |
| 7573 | EQLRDLNIE |
| 7574 | NYARHDILA |
| 7575 | MGQRSAANY |
| 7576 | STARHNMQL |
| 7577 | PAYRMECCE |
| 7578 | ETHRGTVGL |
| 7579 | DVCRKPVSH |
| 7580 | DVMRMEHYL |
| 7581 | PAWRRYCWT |
| 7582 | DMHRGGMWT |
| 7583 | CTLRQIKMH |
| 7584 | YAQRTCWPE |
| 7585 | WCMRDWYQE |
| 7586 | ISQRGTLWC |
| 7587 | RMVRVSVHG |
| 7588 | EAWRNSSSG |
| 7589 | LWDRFCCPV |
| 7590 | NHTRGKQCV |
| 7591 | NCERWFQIE |
| 7592 | QAVRTRTFR |
| 7593 | AYLRRNVGN |
| 7594 | LTNRTQESW |
| 7595 | NVDRQHAQS |
| 7596 | DYERSIENH |
| 7597 | MPTRMQKGA |
| 7598 | MSARINTPD |
| 7599 | CSIRGFSAF |
| 7600 | MTERYYWCQ |
| 7601 | CHIRDCSPI |
| 7602 | NNVRKVGNT |
| 7603 | VEMRANKGL |
| 7604 | WDGRPDLST |
| 7605 | FSYQIQCQN |
| 7606 | TSSEITSPP |
| 7607 | CTKPIPCWM |
| 7608 | IMHEIPEID |
| 7609 | HQSPIHTNT |
| 7610 | REMMIVNCD |
| 7611 | IWPQIQYRM |
| 7612 | PEKWIHMHE |
| 7613 | ILIFIQNCP |
| 7614 | SACNIIYHT |
| 7615 | SGNQIVQAP |
| 7616 | HWELIRCHD |
| 7617 | NSHTIMNGM |
| 7618 | DVDIIGLPA |
| 7619 | QKHMILCHF |
| 7620 | ACEWINMYN |
| 7621 | ATYQIQWPG |
| 7622 | FIIWIEHRH |
| 7623 | GIEQIMGCP |
| 7624 | VIMGIQTDD |
| 7625 | VQPTISYSD |
| 7626 | YSPLIGRWK |
| 7627 | IHSNIPVAE |
| 7628 | NHDLIQFVK |
| 7629 | WSPSIPGGE |
| 7630 | CNYPIIWDK |
| 7631 | VSAYIERQW |
| 7632 | QVSHISEHW |
| 7633 | MMTTIQYHD |
| 7634 | WAEAIKHFT |
| 7635 | VSKFIAASW |
| 7636 | CSNDINGRA |
| 7637 | LNSMIMWGN |
| 7638 | HACVITLDG |
| 7639 | GTEPIMWRD |
| 7640 | LSTLITMMD |
| 7641 | GWALIVNGL |
| 7642 | MVNHILPNN |
| 7643 | RSLDIQYDI |
| 7644 | RVVPIEDSM |
| 7645 | LVNTIWKAA |
| 7646 | GYKFIGRSR |
| 7647 | QYLLIPHLP |
| 7648 | NNGPIWISS |
| 7649 | YGENISPSP |
| 7650 | QSDWISIPC |
| 7651 | CMAHIWEGH |
| 7652 | NWFGINSWH |
| 7653 | FQTSINYYG |
| 7654 | TMEYIGLDQ |
| 7655 | GKIPIDTFR |
| 7656 | WAYYIRLQP |
| 7657 | VAYPIQTDV |
| 7658 | LSIQINNQI |
| 7659 | EQTYISEWG |
| 7660 | TCGDIQLRA |
| 7661 | MAGHIGHHS |
| 7662 | GFKDILMVQ |
| 7663 | VWWMIIHTS |
| 7664 | NAAMIATVC |
| 7665 | SMHLICGEA |
| 7666 | ACSQIGQDD |
| 7667 | DSQEIGSES |
| 7668 | NWEVIGRWH |
| 7669 | HSGFIDRQA |
| 7670 | PVKPIIHAS |
| 7671 | IQAVIASGW |
| 7672 | DHLSICCQN |
| 7673 | CTKPIVMDW |
| 7674 | LNHPIADYD |
| 7675 | TQAKIECCS |
| 7676 | GTEHIPMQA |
| 7677 | SFPMILVNC |
| 7678 | GCQQIQLQT |
| 7679 | ATDMIFVSN |
| 7680 | RQCVIGDYD |
| 7681 | RLAWIEQSP |
| 7682 | YEHPIMWPV |
| 7683 | HEGLIMRLE |
| 7684 | TSYCIQTVD |
| 7685 | AMEMIVMHE |
| 7686 | MGCLIQFCE |
| 7687 | FSWPIHEPD |
| 7688 | ESTHIMKTP |
| 7689 | HPDKIEYWR |
| 7690 | TFGCIESMQ |
| 7691 | EDGLIVYGP |
| 7692 | DHIHIERAN |
| 7693 | GCMCIRHAY |
| 7694 | APIPINMEG |
| 7695 | QMNAIQLCV |
| 7696 | SQSQIRQAD |
| 7697 | PIVLIHYDA |
| 7698 | VADWIGHTP |
| 7699 | GSNSITRDT |
| 7700 | QWEDITKCS |
| 7701 | EDQWISNPF |
| 7702 | QCGFIRLNE |
| 7703 | DSDPITVDL |
| 7704 | EIGIIRHGH |
| 7705 | FQTLILSGL |
| 7706 | DNNMIVRCA |
| 7707 | MAPDIPHCR |
| 7708 | MDADIPHCR |
| 7709 | MDPAIPHCR |
| 7710 | MDPDIAHCR |
| 7711 | MDPDILHCR |
| 7712 | MDPDIPHCC |
| 7713 | MDPDIPHCG |
| 7714 | MDPDIPHCH |
| 7715 | MDPDIPHCL |
| 7716 | MDPDIPHCR |
| 7717 | MDPDIPHCS |
| 7718 | MDPDIPHFR |
| 7719 | MDPDIPHGR |
| 7720 | MDPDIPHWR |
| 7721 | MDPDIPNCR |
| 7722 | MDPDIPPCR |
| 7723 | MDPDIPRCR |
| 7724 | MDPDIPYCR |
| 7725 | MDPDIRHCR |
| 7726 | MDPGIPHCR |
| 7727 | MDPNIPHCR |
| 7728 | MDPVIPHCR |
| 7729 | MDPYIPHCR |
| 7730 | MDTDIPHCR |
| 7731 | MNPDIPHCR |
| 7732 | MYPDIPHCR |
| 7733 | VDPDIPHCR |
| 7734 | HVDQHRVQA |
| 7735 | MYLNYRHFA |
| 7736 | AVASERQLR |
| 7737 | LEQLDRCMY |
| 7738 | LFHQGRFPM |
| 7739 | CVCEMRTVD |
| 7740 | NDCTCRAIM |
| 7741 | TIDVKRWGQ |
| 7742 | EATYTRYMT |
| 7743 | HSGYKRQMK |
| 7744 | TYMEYRQHK |
| 7745 | VDCWLRDPI |
| 7746 | YKTEFRTKL |
| 7747 | DPYNTRFRT |
| 7748 | LNYMRRAMW |
| 7749 | FAIWKRNES |
| 7750 | WKDDLRSTY |
| 7751 | ENQQRRNFG |
| 7752 | IMPPKRYFV |
| 7753 | VAADSRMMI |
| 7754 | IMCHARMMA |
| 7755 | RDIWERPCV |
| 7756 | EHSSPREDN |
| 7757 | EVHLFRGEG |
| 7758 | MYTDMRCWR |
| 7759 | LIHYCRCIN |
| 7760 | HQCQSRSYI |
| 7761 | GTAVSRQSK |
| 7762 | VMFYWRYNS |
| 7763 | FGDHLRVMY |
| 7764 | NSEWLRYTF |
| 7765 | AVHWHRKCE |
| 7766 | PMELWRTWY |
| 7767 | SQDCGRPES |
| 7768 | AAKKGRDLH |
| 7769 | QNLWYRPLT |
| 7770 | TCYCHRAHE |
| 7771 | LACTPRCIF |
| 7772 | VAFGMRWLL |
| 7773 | WAQCDRAVC |
| 7774 | FCDIGRSGG |
| 7775 | GHQWDRLLA |
| 7776 | WLTEKRHFE |
| 7777 | MTGQCRNSQ |
| 7778 | IHPMMRENG |
| 7779 | IFQLSRPAW |
| 7780 | IKETWRVYF |
| 7781 | NTNPGRYGC |
| 7782 | DLVDLRSWM |
| 7783 | CNEVCRNYL |
| 7784 | GFTPDRCRN |
| 7785 | DVPIHRVQL |
| 7786 | TMEAHRFGS |
| 7787 | GSATNRCGN |
| 7788 | NSAECREFQ |
| 7789 | VADTYRMWH |
| 7790 | DYYLNRASH |
| 7791 | NSKACRGCT |
| 7792 | LHNSCRTLE |
| 7793 | TVGSKRDLY |
| 7794 | FNWFVRAYI |
| 7795 | TNIKERTGS |
| 7796 | TTYHHRNTL |
| 7797 | MWTVARMFP |
| 7798 | EQFADRAPR |
| 7799 | LNELPRAEY |
| 7800 | RAEWFRCQH |
| 7801 | PANHARNPK |
| 7802 | SWQQHRKMS |
| 7803 | LPDHWRKFD |
| 7804 | EDWLRRNIW |
| 7805 | DAAMTRTVP |
| 7806 | LLWMGRRRR |
| 7807 | ATCWVRMGQ |
| 7808 | WVKQKDVLC |
| 7809 | MSLDTSVPF |
| 7810 | TSHKMMVFS |
| 7811 | NMKPKDVFR |
| 7812 | LLAKYMVHT |
| 7813 | QDCERQVFA |
| 7814 | PTMLPHVKH |
| 7815 | QGFSTDVKP |
| 7816 | EPLLPAVGN |
| 7817 | IAKDDAVRL |
| 7818 | MENLKGVYD |
| 7819 | QTNQVVVVE |
| 7820 | GTTGHAVNE |
| 7821 | AFLQTPVFS |
| 7822 | ETAFQQVQV |
| 7823 | TWHKQLVGV |
| 7824 | TKYHYHVKV |
| 7825 | RNDWQQVFS |
| 7826 | QVGVTVVQR |
| 7827 | DSHDMFVYK |
| 7828 | MYLSFPVHE |
| 7829 | PWEIEWVYW |
| 7830 | HFADNQVVQ |
| 7831 | MHQFLQVAQ |
| 7832 | NAGMKGVDV |
| 7833 | STQKVPVLA |
| 7834 | GWDMLSVAK |
| 7835 | RNLPTSVEA |
| 7836 | GMSDCEVSF |
| 7837 | GKLTVQVNH |
| 7838 | LILQRNVKD |
| 7839 | NAKEQVVNN |
| 7840 | GDHKCHVEW |
| 7841 | GFKYCEVDH |
| 7842 | MFDMMSVLK |
| 7843 | IVGINHVAW |
| 7844 | MMNQCQVWN |
| 7845 | QGQHVMVHS |
| 7846 | NNCNMNVMK |
| 7847 | EKHFMQVDR |
| 7848 | PIAQDQVWS |
| 7849 | ECHKCEVCV |
| 7850 | PHAYMQVWR |
| 7851 | MQVNLQVIE |
| 7852 | TPQPMNVAT |
| 7853 | AWMCRNVYC |
| 7854 | CGYWLVVPW |
| 7855 | QHTQQLVQN |
| 7856 | CPEHNQVGI |
| 7857 | HDEKQKVYG |
| 7858 | EPFLKEVNV |
| 7859 | AVTQLSVER |
| 7860 | QHKTKCVDN |
| 7861 | IHCNVLVNY |
| 7862 | MDGHFSVGC |
| 7863 | YHQLVQVNE |
| 7864 | GQPVWCVCM |
| 7865 | MIKPLLVVG |
| 7866 | GLGWWNVHT |
| 7867 | TMAQWTVHN |
| 7868 | EPMPMPVWN |
| 7869 | IPTFDGVEP |
| 7870 | ELPDTEVPW |
| 7871 | QAMLHHVWV |
| 7872 | FTEWKPVFF |
| 7873 | QAVTLEVVC |
| 7874 | HECDTVVVN |
| 7875 | CVNLMAVNN |
| 7876 | QHQAFFVFT |
| 7877 | ATEKQMVDH |
| 7878 | TGMHPFVRG |
| 7879 | QVMQEKVRC |
| 7880 | DFPHMHVGT |
| 7881 | NAGVVKVWP |
| 7882 | EITQHFVKH |
| 7883 | VADTWQVHC |
| 7884 | SIYQKWVTN |
| 7885 | PIKGVAVQE |
| 7886 | IFSHPSVSG |
| 7887 | ECSVHPVNC |
| 7888 | DKMSLQVQI |
| 7889 | EGEWLIVPV |
| 7890 | TQNACKVRT |
| 7891 | TGSLTDVIW |
| 7892 | GSAMLMVIR |
| 7893 | TAGEWIVQH |
| 7894 | TGMSRYVQD |
| 7895 | FAGFTPVWM |
| 7896 | NMWYSFVMI |
| 7897 | ETEHGSVYT |
| 7898 | PSILPVVMF |
| 7899 | PEYVKWVAD |
| 7900 | TIHPAWVTL |
| 7901 | QWFTKGVDE |
| 7902 | CYAEEPVWP |
| 7903 | QIMSAKVFE |
| 7904 | MTTFCDVEA |
| 7905 | FTGTPPVDM |
| 7906 | ESWMCPVDY |
| 7907 | DCTYCCVKA |
| 7908 | AVVEHAVNR |
| 7909 | MDYHCQVED |
| 7910 | QPGVVPVPC |
| 7911 | MPYPLNVKE |
| 7912 | NVYATPVVC |
| 7913 | NCHIDHVQD |
| 7914 | VDMGVHVGS |
| 7915 | VIKVNTVNP |
| 7916 | DFKALDVQC |
| 7917 | MSAQRYVQT |
| 7918 | FCKCFMKHC |
| 7919 | VIPEFMNHA |
| 7920 | AVIHEKPHG |
| 7921 | MLFDFEEHN |
| 7922 | RCVQAHFHK |
| 7923 | NPTDLFQHE |
| 7924 | GEMTRCFHN |
| 7925 | IAVASHAHG |
| 7926 | WKWYGLPHH |
| 7927 | SVTYHEWHP |
| 7928 | TDHPWVLHG |
| 7929 | AKEHMPHHM |
| 7930 | GIHQLIHHC |
| 7931 | EACMPWAHH |
| 7932 | DVMQYQNHM |
| 7933 | DVDFLKMHC |
| 7934 | MYAGWLNHG |
| 7935 | TNGVQIMHV |
| 7936 | DGLSYEAHD |
| 7937 | EHCYYKEHE |
| 7938 | HPSWPTMHK |
| 7939 | ISCIMKKHL |
| 7940 | IWGTRSAHR |
| 7941 | NQCWCPKHY |
| 7942 | SFKWCPGHE |
| 7943 | ADCFRPFHI |
| 7944 | IQDCSTLHW |
| 7945 | LQHFDLIHL |
| 7946 | PQQCGGFHR |
| 7947 | TWGLHHYHT |
| 7948 | VNPPDMIHT |
| 7949 | DKANKMMHG |
| 7950 | ETMSEGIHC |
| 7951 | FYPYTNCHH |
| 7952 | PAAPLILHY |
| 7953 | QCLNMMMHG |
| 7954 | QVGTGMYHS |
| 7955 | WDHYLPEHV |
| 7956 | TVQDNDEHA |
| 7957 | TKQFFTAHC |
| 7958 | NFSVCPRHL |
| 7959 | ITTDATGHF |
| 7960 | STFHFQSHD |
| 7961 | HMDHCQCHE |
| 7962 | QYWWPATHD |
| 7963 | LGEPYKAHD |
| 7964 | ISNTMVEHG |
| 7965 | VTAFTACHY |
| 7966 | EWLSLYCHW |
| 7967 | PIHTYKRHP |
| 7968 | GYNYYWQHA |
| 7969 | ATVHMPKHE |
| 7970 | MFSAFSMHD |
| 7971 | TCTNSEPHP |
| 7972 | TIDWCEPHC |
| 7973 | LMHGVMDHV |
| 7974 | YITCQYSHH |
| 7975 | LATTTSYHC |
| 7976 | GTCLFKTHK |
| 7977 | DSAKQWHHQ |
| 7978 | SYAAFLHHG |
| 7979 | PNCDPAHHD |
| 7980 | FAPEFDNHC |
| 7981 | QISMKGEHG |
| 7982 | QLTTNDAHG |
| 7983 | CMAQMVYHH |
| 7984 | CIDVLFKHL |
| 7985 | EAGKEGAHF |
| 7986 | QYCNTDNHI |
| 7987 | YLESKTIHA |
| 7988 | PHQYGDEHQ |
| 7989 | WPTMLSAHM |
| 7990 | DQFCPFDHY |
| 7991 | LPTQVYGHN |
| 7992 | DTHPMSNHR |
| 7993 | MGVITSIHQ |
| 7994 | NAMTCSMHN |
| 7995 | TIQHAFKHN |
| 7996 | NVFPAETHV |
| 7997 | TLEPAYRHA |
| 7998 | YEICKHGHF |
| 7999 | HSLVQQLHP |
| 8000 | VPVATMIHH |
| 8001 | RVDGADRHY |
| 8002 | CHILDGLHD |
| 8003 | MLGVAPGHL |
| 8004 | ANLISQGHC |
| 8005 | GSYIVQNHC |
| 8006 | WPEAFHLHQ |
| 8007 | CDFMFTRHI |
| 8008 | HVWNDVFHM |
| 8009 | ADYSMMWHM |
| 8010 | WNWLKVMHE |
| 8011 | PVTTKNCHE |
| 8012 | FCHEVFSHT |
| 8013 | IHKMNFDHL |
| 8014 | WAIFGDIHH |
| 8015 | QSGTCHYHD |
| 8016 | WVYSHEKHY |
| 8017 | DVKYATLHD |
| 8018 | GTEMMGDHA |
| 8019 | QTKVHQQHT |
| 8020 | ASSSQYPHN |
| 8021 | TSLCAKPHF |
| 8022 | GYTQQDEHF |
| 8023 | DHCHCIAHP |
| 8024 | IPACGIMHE |
| 8025 | TCNVSWKHE |
| 8026 | TKGMWQQHS |
| 8027 | SAGPFFDHY |
| 8028 | HTVNAESHM |
| 8029 | DDGSNTMHG |
| 8030 | GVAQYLHHC |
| 8031 | EFADYNFHF |
| 8032 | NTCEGWSHF |
| 8033 | FPKCAIWHY |
| 8034 | AFGLTTGHT |
| 8035 | STGSFSIHC |
| 8036 | HHEFTNSHA |
| 8037 | QVVIDPAHY |
| 8038 | CAGMAWSHS |
| 8039 | TVQWNFAHM |
| 8040 | RCVQDHFHK |
| 8041 | LLWMGQRHR |
| 8042 | QQTWEQMQR |
| 8043 | CAMPHYQVR |
| 8044 | LGKVQTHIR |
| 8045 | CSVWMQRDR |
| 8046 | VFPKDEQFR |
| 8047 | EHITEQDLR |
| 8048 | DKTVQTSAR |
| 8049 | GGMWQCEVR |
| 8050 | LEKNSSQMR |
| 8051 | CDAEAEFRR |
| 8052 | MGGSCKQER |
| 8053 | TCVIGKGRR |
| 8054 | VAFFWQRCR |
| 8055 | FAESMWCDR |
| 8056 | FAQQDKQDR |
| 8057 | DANWTVRRR |
| 8058 | MCNGGIPAR |
| 8059 | EGATAWQDR |
| 8060 | SHIQLNAWR |
| 8061 | IPAEWDSWR |
| 8062 | DAHNDALAR |
| 8063 | QPQSRNGAR |
| 8064 | NWNHCHQWR |
| 8065 | CWHFFGADR |
| 8066 | CAMDNTMCR |
| 8067 | TFVYGARDR |
| 8068 | QLDTYMEGR |
| 8069 | AATTHHSAR |
| 8070 | YTNHYYNAR |
| 8071 | VGIWFIPCR |
| 8072 | WSEDMSQYR |
| 8073 | MCSQTISMR |
| 8074 | RGLAQDCCR |
| 8075 | FCNKLTSVR |
| 8076 | PTNYGWMWR |
| 8077 | HTLQWEAVR |
| 8078 | QCAHFYQTR |
| 8079 | EWEGVSQFR |
| 8080 | QAVIAVDGR |
| 8081 | GITEQPLAR |
| 8082 | GIVMLKCGR |
| 8083 | TLLLVGKDR |
| 8084 | GLWLNVAPR |
| 8085 | QTEMKKCAR |
| 8086 | GHIDLEIMR |
| 8087 | RMTPNNCQR |
| 8088 | TCGPKLTQR |
| 8089 | HIGWMGWLR |
| 8090 | QETHWAMMR |
| 8091 | PYHNPWECR |
| 8092 | YDLWGWSTR |
| 8093 | QHYNSMIDR |
| 8094 | TYMTNLGDR |
| 8095 | GKLTFGTER |
| 8096 | GVHDPLHDR |
| 8097 | TTYLSMRDR |
| 8098 | AHMNYCLSR |
| 8099 | NYCKYNKMR |
| 8100 | GVGPSHGER |
| 8101 | AKISATWDR |
| 8102 | INMEYLTDR |
| 8103 | YTGHTNSGR |
| 8104 | MHCLHQTMR |
| 8105 | YWCPSQWWR |
| 8106 | MLAFDPMGR |
| 8107 | IIAVSMRGR |
| 8108 | VWEDNPPFR |
| 8109 | CTLMHSHTR |
| 8110 | LLWMEQRRR |
| 8111 | LLWMGQQRR |
| 8112 | LLWMGQRCR |
| 8113 | LLWMGQRRR |
| 8114 | LLWMGQWRR |
| 8115 | MDPDMPHCR |
| 8116 | MDPDSPHCR |
| 8117 | MDPDVPHCR |

### Example 6

AAV5 Variants with Tissue Tropism in Spleen

This example describes engineered AAV5 variants with tissue tropism in spleen that were discovered using the methods and systems described in EXAMPLE 1-EXAMPLE 2.

CNS, liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive spleen tropism. The results are shown in FIG. 18F which shows the positional frequency of each amino acid in the 581 to 589 region of AAV5 VP1 in spleen tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in spleen over the frequency of that given amino acid residue occurring at the specified position in variants identified in all other harvested tissues was analyzed to identify a set of sequence rules for capsids that preferentially target spleen tissue. With reference to TABLE 11 below, and SEQ ID NO: 2 (AAV5 VP1), the following amino acids can, thus, be independently mutated, in any combination, at any one or more positions Xaa1-Xaa9, to provide a VP1 capsid with enhanced spleen tropism as compared wildtype AAV5 VP1 capsid (SEQ ID NO: 1), where spleen tropism here refers to properties that are preferred for spleen transduction over properties that are preferred for transduction of all other harvested tissues.

TABLE 11
Spleen Tropism Rules

Xaa1 is selected from C, F, H, I, L, P, W, or Y
Xaa1 is selected from C, F, P, W, or Y
Xaa1 is selected from P, W, or Y
Xaa1 is P
Xaa2 is selected from D, E, L, N, P, R, or W
Xaa2 is selected from D, E, or W
Xaa2 is D
Xaa3 is selected from C, D, E, P, or W
Xaa3 is selected from D, P, or W
Xaa3 is P
Xaa4 is selected from C, F, G, H, R, W or Y
Xaa4 is selected from C, H, or W
Xaa4 is C
Xaa5 is selected from A, D, E, G, P, R, or W
Xaa5 is selected from D, E, G, or P
Xaa5 is D
Xaa6 is selected from A, C, D, E, K, R, W
Xaa6 is selected from C, K, or R
Xaa6 is K
Xaa7 is selected from F, L, P, R, W, Y
Xaa7 is selected from L, P, or W
Xaa7 is P
Xaa8 is selected from E, I, K, L, P, R, or T
Xaa8 is selected from P, R, or K
Xaa8 is K
Xaa9 is selected from C, H, M, T, V, or W
Xaa9 is selected from C, T, or V
Xaa9 is V

TABLE 12 below provides sequences of the 581 to 589 region for variant AAV VP1 capsid polypeptides that were found in spleen tissue and comport to one or more of the rules provided in TABLE 11. The present disclosure, thus, provides for rAAVs composed of engineered AAV5 VP1 capsid polypeptides having a SEQ ID NO: 2, wherein the Xaa1 to Xaa9 region is any one of SEQ ID NO: 37438-SEQ ID NO: 38437, as disclosed in TABLE 12. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 12
Sequences of the 581 to 589 Region in AAV5 VP1
Capsid Polypeptide that
Drive Spleen Tissue Tropism

| SEQ ID | 581-589 |
|---|---|
| NO | Sequence |
| 37438 | PANFGQAYP |
| 37439 | PCVQDLRAI |
| 37440 | PCYGQTDWN |
| 37441 | PDAQNEMWH |
| 37442 | PDPRPATND |
| 37443 | PDTGHEWWW |
| 37444 | PDWREATVV |
| 37445 | PEDPTVTNH |
| 37446 | PEKHSCSTT |
| 37447 | PFILKQRNQ |
| 37448 | PGMTTTPSF |
| 37449 | PGSLECAAL |
| 37450 | PHVANMGPG |
| 37451 | PHVNYDTLC |
| 37452 | PIWGNCAKT |
| 37453 | PKGSQYGEQ |
| 37454 | PKGVLLGGA |
| 37455 | PKLQYHSES |
| 37456 | PLARIEPLW |
| 37457 | PLECKMWYN |
| 37458 | PMPFCKHQF |
| 37459 | PMQQKLWME |
| 37460 | PMRWNKFVP |
| 37461 | PMVKLEETS |
| 37462 | PNCKHSIAV |
| 37463 | PNTCTKDGL |
| 37464 | PPHRFRHIP |
| 37465 | PPWPPETAE |
| 37466 | PQASKAFSK |
| 37467 | PQQQYLSVC |
| 37468 | PQTAYMQAY |
| 37469 | PRYFVEACE |
| 37470 | PSHWFCGGA |
| 37471 | PTDFIRVNE |
| 37472 | PTRATMFID |
| 37473 | PVAWRENSL |
| 37474 | PVTRFECCK |
| 37475 | PYCICTQPY |
| 37476 | PYHDNSAPQ |
| 37477 | PYHLNQDNN |
| 37478 | PWAIEYSLV |
| 37479 | PYIMEFHPF |
| 37480 | PKEHFPTCM |
| 37481 | PAAAMFLPS |
| 37482 | PAAHKEWEW |
| 37483 | PAAHNACHC |
| 37484 | PAALDLYNG |
| 37485 | PACLGRTDA |
| 37486 | PACNCQIAS |
| 37487 | PACRKGYWN |
| 37488 | PADEAFVEC |
| 37489 | PADKSQLGA |
| 37490 | PADLNLSIP |
| 37491 | PADNLHCHG |
| 37492 | PADSAKQRK |
| 37493 | PADYGWCVG |
| 37494 | PADYRAYGD |
| 37495 | PAECKYIYA |
| 37496 | PAEFPFMIH |
| 37497 | PAEFRCTPF |
| 37498 | PAEPGQSMG |
| 37499 | PAEVTNFGV |
| 37500 | PAEWCLQHV |
| 37501 | PAFGCMLNH |
| 37502 | PAGCHGKTH |
| 37503 | PAGCVCASS |
| 37504 | PAGMAMPGT |
| 37505 | PAGNAAWTT |
| 37506 | PAGWNECAC |
| 37507 | PAHKVDLCF |
| 37508 | PAHQCTVHD |
| 37509 | PAHYVPGIE |
| 37510 | PAISNKFAG |
| 37511 | PAKLYITVR |
| 37512 | PAKQAADYG |
| 37513 | PAKYLPWQY |
| 37514 | PALRETPWT |
| 37515 | PALWRYYKQ |
| 37516 | PAMMMPTKQ |
| 37517 | PAMRYDSLY |
| 37518 | PANNCRIHT |
| 37519 | PAPCWWNVT |
| 37520 | PAPDNSDTC |
| 37521 | PAPHAWDVH |
| 37522 | PAPIWQVLD |
| 37523 | PAPKHSYQN |
| 37524 | PAPPLAQSN |
| 37525 | PAQNAPMCG |
| 37526 | PAQQLTCIK |
| 37527 | PARCAQFVA |
| 37528 | PAREDGNGW |
| 37529 | PARKAYCLP |
| 37530 | PARLISDYE |
| 37531 | PARSLWQTY |
| 37532 | PARSTYGAA |
| 37533 | PASCMWFSH |
| 37534 | PASLGIPKM |
| 37535 | PATEVQQFM |
| 37536 | PATNQCKRI |
| 37537 | PATQERGEK |
| 37538 | PAVADDMYK |
| 37539 | PAVHMQLDP |
| 37540 | PAVNANATN |
| 37541 | PAVNVPMST |
| 37542 | PAVQCSWHG |
| 37543 | PAVQGLPKM |
| 37544 | PAVSACPYW |
| 37545 | PAYGSPPWA |
| 37546 | PCADTMSVW |
| 37547 | PCAFETTQH |
| 37548 | PCAGKDICN |
| 37549 | PCAPYARSK |
| 37550 | PCAQWYCDC |
| 37551 | PCCEDVSNI |
| 37552 | PCCEGTMDN |
| 37553 | PCCQICNTW |
| 37554 | PCCYTQRPT |
| 37555 | PCDRTCPDM |
| 37556 | PCEFKSTET |
| 37557 | PCEGHEKPQ |
| 37558 | PCERALLQH |
| 37559 | PCEVQNAQT |
| 37560 | PCFAVMAGC |
| 37561 | PCFCHYTNC |
| 37562 | PCFGPSYAD |
| 37563 | PCFVKDSTA |
| 37564 | PCGGLTHPH |
| 37565 | PCGLMDDYP |
| 37566 | PCGQWKIQN |
| 37567 | PCHAFEPDH |
| 37568 | PCHMMVIAN |
| 37569 | PCHQQELDK |
| 37570 | PCHQYWIED |
| 37571 | PCHRYSKFM |
| 37572 | PCKCYPRKC |
| 37573 | PCLHMQYNP |
| 37574 | PCLHTCHRG |
| 37575 | PCLWPMGCY |
| 37576 | PCMHVQQWE |
| 37577 | PCMKKHSST |
| 37578 | PCMQNFDDG |
| 37579 | PCNKWNPKQ |
| 37580 | PCNLMRDQN |
| 37581 | PCNLVWNCE |
| 37582 | PCNTDPSIH |
| 37583 | PCPDWYVMS |
| 37584 | PCPHDRLNA |
| 37585 | PCPIIWESG |
| 37586 | PCPLSQYQT |
| 37587 | PCPSEDWFW |
| 37588 | PCQSIKCCW |
| 37589 | PCQSSWLMI |
| 37590 | PCRVGFSYE |
| 37591 | PCSESWSNT |
| 37592 | PCSHYCART |
| 37593 | PCSWFYHGG |
| 37594 | PCSYTIFDT |
| 37595 | PCTCCFNRQ |
| 37596 | PCTCVWYRC |
| 37597 | PCTGQLFMA |
| 37598 | PCTQKQKPF |
| 37599 | PCTWAHSHD |
| 37600 | PCVHGTQTW |
| 37601 | PCVKKDCEE |
| 37602 | PCVNKVGTM |
| 37603 | PCVWQWHCT |
| 37604 | PCWLKPTSW |
| 37605 | PCWRRDIHP |
| 37606 | PCWVNDHPM |
| 37607 | PCWWKWMCG |
| 37608 | PCYLCMLIL |
| 37609 | PCYWAIAGV |
| 37610 | PCYYFGMPH |
| 37611 | PDACSPFGA |
| 37612 | PDADQFVPQ |
| 37613 | PDAGWQNSP |
| 37614 | PDAMKDCVT |
| 37615 | PDANIEFLC |
| 37616 | PDAPWEGWH |
| 37617 | PDCCDMQCF |
| 37618 | PDCTERFME |
| 37619 | PDDEQVRVN |
| 37620 | PDDEVCSKV |
| 37621 | PDDGHKHPF |
| 37622 | PDDMWQYEA |
| 37623 | PDEEYAQYW |
| 37624 | PDEGFLYDN |
| 37625 | PDELHLEDG |
| 37626 | PDEPRSKCQ |
| 37627 | PDEQHMGYW |
| 37628 | PDFQRENFE |
| 37629 | PDGFGVSMP |
| 37630 | PDGRRYVCS |
| 37631 | PDHAMEYQC |
| 37632 | PDHEPNGDA |
| 37633 | PDHGIQFGW |
| 37634 | PDHIRMVTP |
| 37635 | PDHKLIVYD |
| 37636 | PDHMGLPYH |
| 37637 | PDHVHCIHA |
| 37638 | PDHVREHCM |
| 37639 | PDIHGPQSE |
| 37640 | PDKACSHHP |
| 37641 | PDKDNHVAY |
| 37642 | PDKSEAQRQ |
| 37643 | PDKVSDYQE |
| 37644 | PDLQPRDTT |
| 37645 | PDLVCDYCQ |
| 37646 | PDLVPIVGN |
| 37647 | PDLWSPFWY |
| 37648 | PDMFKERSQ |
| 37649 | PDMIVDPDN |
| 37650 | PDMLTDSTC |
| 37651 | PDMPHDRHC |
| 37652 | PDMQWNGEW |
| 37653 | PDMTCTINI |
| 37654 | PDNIFWGEA |
| 37655 | PDNLAPYVP |
| 37656 | PDNMITMQH |
| 37657 | PDNNFLSNQ |
| 37658 | PDPGHRMYA |
| 37659 | PDPHCEDRK |
| 37660 | PDPHIVWCG |
| 37661 | PDPLARRFE |
| 37662 | PDQHTHLAM |
| 37663 | PDQRGQTGQ |
| 37664 | PDRPCEDVN |
| 37665 | PDSCLHRTA |
| 37666 | PDSVPVQHN |
| 37667 | PDTAKQDMS |
| 37668 | PDTCWNWPR |
| 37669 | PDTSMECDH |
| 37670 | PDTTMKNTL |
| 37671 | PDWATLMGN |
| 37672 | PDWAVMKAP |
| 37673 | PDWFEENTV |
| 37674 | PDWGSHYCN |
| 37675 | PDWHASYGN |
| 37676 | PDWNMRMYP |
| 37677 | PDWRYGWYS |
| 37678 | PDWSGKYDI |
| 37679 | PDWSPPIQN |
| 37680 | PDWWAIEMG |
| 37681 | PDYHIGCNH |
| 37682 | PDYMDCAPM |
| 37683 | PDYWKSAFA |
| 37684 | PEAKWKEHD |
| 37685 | PEAMIGQEN |
| 37686 | PEANEVQTR |
| 37687 | PEARSWWAQ |
| 37688 | PEAVWCAFA |
| 37689 | PECAESPTT |
| 37690 | PECFLDSNV |
| 37691 | PECFSPENM |
| 37692 | PECLINYTC |
| 37693 | PECQSIDSV |
| 37694 | PECSNWRNH |
| 37695 | PECVIEYED |
| 37696 | PECVRYSKC |
| 37697 | PEDCCRQDS |
| 37698 | PEDMTLDGS |
| 37699 | PEDNNIRRN |
| 37700 | PEDSGYHWH |
| 37701 | PEEANQQVT |
| 37702 | PEEAPKQMF |
| 37703 | PEEETMYAC |
| 37704 | PEEHEVEQT |
| 37705 | PEEINDHCY |
| 37706 | PEEYEHMNC |
| 37707 | PEFFHWPHS |
| 37708 | PEFMSKGDS |
| 37709 | PEFVQYGSC |
| 37710 | PEGCTYPQL |
| 37711 | PEGHEEHPS |
| 37712 | PEGLVIHMT |
| 37713 | PEHGIHDGE |
| 37714 | PEHRAANVG |
| 37715 | PEHSNLFSY |
| 37716 | PEHTVIWES |
| 37717 | PEHVETHGQ |
| 37718 | PEIFGQMSW |
| 37719 | PEIIWQIAT |
| 37720 | PEITCKIAD |
| 37721 | PEIVNPARF |
| 37722 | PEKVLLGMV |
| 37723 | PELYTMFFT |
| 37724 | PEMALTKGL |
| 37725 | PEMCDPADQ |
| 37726 | PEMDKFKNM |
| 37727 | PEMGEATKV |
| 37728 | PEMPRHICR |
| 37729 | PEMTYEPMS |
| 37730 | PEMVEEMNV |
| 37731 | PEMWMILEG |
| 37732 | PENHKMGPW |
| 37733 | PENTFCSTY |
| 37734 | PENVGASGA |
| 37735 | PENWRPWVT |
| 37736 | PEPKLIGLF |
| 37737 | PEPLGQGTW |
| 37738 | PEPQTQDDG |
| 37739 | PEPRNVMKS |
| 37740 | PEQCSERIV |
| 37741 | PEQNDVNGM |
| 37742 | PEQRRRYDG |
| 37743 | PEQWSWGVC |
| 37744 | PEQWWRYQA |
| 37745 | PEQYKYYQC |
| 37746 | PEQYNAQAE |
| 37747 | PERINHLGC |
| 37748 | PERLFTCFV |
| 37749 | PERNKVWVA |
| 37750 | PERYNQNQL |
| 37751 | PESCGAAAP |
| 37752 | PESDRIYRP |
| 37753 | PESIKTIQR |
| 37754 | PESNKWEDH |
| 37755 | PESNWRCPE |
| 37756 | PETAAAKAM |
| 37757 | PETFDTYPH |
| 37758 | PETHSESCN |
| 37759 | PETWNDRCH |
| 37760 | PETYLGIPD |
| 37761 | PEVIQNESM |
| 37762 | PEVPSTNHA |
| 37763 | PEVTSRDIM |
| 37764 | PEVWGGRTQ |
| 37765 | PEWGHIGNH |
| 37766 | PEWIDGYVM |
| 37767 | PEWWGQNIH |
| 37768 | PEYHWDHYY |
| 37769 | PEYSTSFEQ |
| 37770 | PEYTEFEHQ |
| 37771 | PFAKPNIEE |
| 37772 | PFCCEMHIP |
| 37773 | PFCLQDVCH |
| 37774 | PFDTCFICT |
| 37775 | PFDVDGPIY |
| 37776 | PFEAQINFD |
| 37777 | PFECHKSSS |
| 37778 | PFESHDQYT |
| 37779 | PFFCSGVKC |
| 37780 | PFFEPDDEM |
| 37781 | PFGLVEINT |
| 37782 | PFGRMIVDD |
| 37783 | PFGWESQQP |
| 37784 | PFHEDCPTE |
| 37785 | PFHEPWAYA |
| 37786 | PFHGVILEN |
| 37787 | PFHLPRFPS |
| 37788 | PFHVHGEHY |
| 37789 | PFHYNKTLC |
| 37790 | PFKAHKPSL |
| 37791 | PFKHENLRH |
| 37792 | PFKPCPIEI |
| 37793 | PFKSDFWAI |
| 37794 | PFLAGDRPV |
| 37795 | PFLMEENYR |
| 37796 | PFLYVIDRR |
| 37797 | PFMAHGMYQ |
| 37798 | PFMISNALQ |
| 37799 | PFMQCCYSW |
| 37800 | PFNFMGMVQ |
| 37801 | PFNPDWGRF |
| 37802 | PFNRMTDYY |
| 37803 | PFNSDSYIR |
| 37804 | PFNYMFLIM |
| 37805 | PFPAMKIKL |
| 37806 | PFPWEWLRL |
| 37807 | PFQQFLERC |
| 37808 | PFQWVWREQ |
| 37809 | PFRECKNCT |
| 37810 | PFSFSQPGS |
| 37811 | PFSKGAYLA |
| 37812 | PFSQWIHSA |
| 37813 | PFTHPICWH |
| 37814 | PFTHQPAGM |
| 37815 | PFVGLLEHS |
| 37816 | PFVKPLDIA |
| 37817 | PFVQYKWDR |
| 37818 | PFWCEAISY |
| 37819 | PFWIHRMIE |
| 37820 | PFWLFHAKC |
| 37821 | PFWVRITPE |
| 37822 | PFYDQKLSF |
| 37823 | PFYKKYTAF |
| 37824 | PFYPLWENF |
| 37825 | PFYQHLSGY |
| 37826 | PFYTSDGCP |
| 37827 | PFYYFTCCQ |
| 37828 | PGADTHGPQ |
| 37829 | PGAGLGPPS |
| 37830 | PGANYWGCM |
| 37831 | PGAYKNQQH |
| 37832 | PGCLEIFHM |
| 37833 | PGCPNSNPH |
| 37834 | PGDCEEAII |
| 37835 | PGDCHEAWC |
| 37836 | PGDQYREWC |
| 37837 | PGDRGPIGR |
| 37838 | PGDWQTTPT |
| 37839 | PGDYRQTLP |
| 37840 | PGEAAFEKQ |
| 37841 | PGEAVCHRW |
| 37842 | PGEFKGLVS |
| 37843 | PGFGMNSIR |
| 37844 | PGGAYVWTR |
| 37845 | PGGCMWARD |
| 37846 | PGGGFCLNG |
| 37847 | PGGINNMTV |
| 37848 | PGGWLEHPA |
| 37849 | PGGWQVNPS |
| 37850 | PGHCYRMGW |
| 37851 | PGHFWWQHA |
| 37852 | PGHKFRSMD |
| 37853 | PGHWNLYHQ |
| 37854 | PGHYAIMKR |
| 37855 | PGIHSVMNQ |
| 37856 | PGIRAQCGE |
| 37857 | PGKLQTMGP |
| 37858 | PGKMINPQM |
| 37859 | PGKNLKWTD |
| 37860 | PGKVAWSQG |
| 37861 | PGLICSIDW |
| 37862 | PGLPYRNHH |
| 37863 | PGMEKNGDL |
| 37864 | PGNCIPAMG |
| 37865 | PGNEHMQMT |
| 37866 | PGNGRLYWT |
| 37867 | PGNRFMTYY |
| 37868 | PGNWAKPGG |
| 37869 | PGPHDMGSL |
| 37870 | PGPSDNTPN |
| 37871 | PGQGSDTEE |
| 37872 | PGQLTSRHF |
| 37873 | PGQSDSDMV |
| 37874 | PGQTEERVW |
| 37875 | PGRIDFWKP |
| 37876 | PGRPMWMTN |
| 37877 | PGRYCKTHS |
| 37878 | PGSQALQWA |
| 37879 | PGSSNKPYA |
| 37880 | PGSSSACLC |
| 37881 | PGSTYQAML |
| 37882 | PGSWYMHLP |
| 37883 | PGTFWHEQY |
| 37884 | PGTGVADRN |
| 37885 | PGTKACMKH |
| 37886 | PGTSWIPWM |
| 37887 | PGVTNCDVG |
| 37888 | PGVTVWGYH |
| 37889 | PGVWLSYNC |
| 37890 | PGWGKSFLT |
| 37891 | PGWHNFTAI |
| 37892 | PGWLEAWSP |
| 37893 | PGWPAPTGH |
| 37894 | PGYKTPWAK |
| 37895 | PHAEGLFCT |
| 37896 | PHAHPMFMT |
| 37897 | PHASSPMQP |
| 37898 | PHCFPAYCC |
| 37899 | PHCKICRWN |
| 37900 | PHCLADYFM |
| 37901 | PHCPMIRQD |
| 37902 | PHCREGEVQ |
| 37903 | PHCWWKWSG |
| 37904 | PHDMSMRFY |
| 37905 | PHDTNNAKY |
| 37906 | PHEEGNKWG |
| 37907 | PHEGHDRKQ |
| 37908 | PHEHKEGVC |
| 37909 | PHENEAIHW |
| 37910 | PHEYIVVKS |
| 37911 | PHFKHHKTQ |
| 37912 | PHFKMEPHD |
| 37913 | PHFSRQNGT |
| 37914 | PHGGQRISN |
| 37915 | PHGLNEAAI |
| 37916 | PHHAGTQRS |
| 37917 | PHHDVSIPC |
| 37918 | PHHFTVYPF |
| 37919 | PHHIMWHHP |
| 37920 | PHHLMSCIG |
| 37921 | PHIEVAIGH |
| 37922 | PHIIPLEGL |
| 37923 | PHIKDWPRA |
| 37924 | PHISCAHAW |
| 37925 | PHKFIFMTS |
| 37926 | PHKPGTTKD |
| 37927 | PHKWIKFAG |
| 37928 | PHKWWNVHW |
| 37929 | PHMCYLLWR |
| 37930 | PHMWRDNHN |
| 37931 | PHMYKWTRM |
| 37932 | PHNCELNSA |
| 37933 | PHNQREHCA |
| 37934 | PHNYDWGCS |
| 37935 | PHPENMSKQ |
| 37936 | PHPFGHREA |
| 37937 | PHPHKQHMF |
| 37938 | PHPQCTHMM |
| 37939 | PHPRMGIQQ |
| 37940 | PHQCKQLTS |
| 37941 | PHRANSTQY |
| 37942 | PHRFPDRKM |
| 37943 | PHSCCCMKC |
| 37944 | PHSCLFMWA |
| 37945 | PHSEQIKCG |
| 37946 | PHSFNTIMS |
| 37947 | PHSIVATRD |
| 37948 | PHSNQAEFN |
| 37949 | PHSWAYHTS |
| 37950 | PHTMRVCLP |
| 37951 | PHTPQGTKA |
| 37952 | PHTREGLAQ |
| 37953 | PHTWIKQEG |
| 37954 | PHVMGGEWC |
| 37955 | PHVPHEIGI |
| 37956 | PHVRCPMDL |
| 37957 | PHWDKGYDL |
| 37958 | PHWFFCNMN |
| 37959 | PHWMPWNQV |
| 37960 | PHWQYETGS |
| 37961 | PHWRPPLGF |
| 37962 | PHYDSLAHS |
| 37963 | PHYLQVCCA |
| 37964 | PIAEDFVAV |
| 37965 | PIANDMHTY |
| 37966 | PIANKHPSI |
| 37967 | PIAPSGFVG |
| 37968 | PIAWVEQFP |
| 37969 | PICFEDCCH |
| 37970 | PICMIGRWT |
| 37971 | PIDEAINDS |
| 37972 | PIDPRQSGE |
| 37973 | PIDPVYHLI |
| 37974 | PIEMHVACS |
| 37975 | PIESPIHHS |
| 37976 | PIESTPVLV |
| 37977 | PIFMTEQNL |
| 37978 | PIGPSSTSC |
| 37979 | PIHHKQTYM |
| 37980 | PIHRESPRI |
| 37981 | PIIPQAQLF |
| 37982 | PIITNCAHS |
| 37983 | PIIWELGWA |
| 37984 | FDAGEEAYF |
| 37985 | FDLDQQVHT |
| 37986 | FDPQDDWCY |
| 37987 | FDRISVLGV |
| 37988 | FDTDHRWCW |
| 37989 | GDDLYVHGM |
| 37990 | GDECFCKLI |
| 37991 | GDFCFYKMT |
| 37992 | GDFHPDPLC |
| 37993 | GDGKSTKVT |
| 37994 | GDVHHLHSK |
| 37995 | GDVPTWPKC |
| 37996 | HDTSQAHNI |
| 37997 | IDEDIFFAA |
| 37998 | IDIHMSANI |
| 37999 | IDKNWKGLW |
| 38000 | IDQCAKFHR |
| 38001 | IDTFNCDTY |
| 38002 | IDWFAGACG |
| 38003 | KDAAYVTPD |
| 38004 | KDHCYTCID |
| 38005 | KDPRITWYV |
| 38006 | KDQFWGCLP |
| 38007 | LDHPMHMMC |
| 38008 | MDAHGRNHG |
| 38009 | MDHPRVEMQ |
| 38010 | MDLYYTLAN |
| 38011 | MDNFDRPAA |
| 38012 | MDNRELVVK |
| 38013 | MDQCQQHTC |
| 38014 | NDEKAAIDI |
| 38015 | NDGSSVHMN |
| 38016 | NDKHTTMEN |
| 38017 | NDPYNKQSQ |
| 38018 | QDHTNAWHQ |
| 38019 | QDQDLNAWA |
| 38020 | QDRDQSYHR |
| 38021 | RDAANLEFP |
| 38022 | RDDLMVKIV |
| 38023 | RDIDHKNDE |
| 38024 | SDANDRADW |
| 38025 | SDCAMTIHV |
| 38026 | SDCSDFTQF |
| 38027 | SDDHAMQMN |
| 38028 | SDDVKLPGL |
| 38029 | SDHTCCWTF |
| 38030 | SDMDPCSDK |
| 38031 | SDMWMERSC |
| 38032 | SDNCMEADN |
| 38033 | SDRNALMPN |
| 38034 | SDTKWNVCY |
| 38035 | TDDFGTPWD |
| 38036 | TDEHQGVFQ |
| 38037 | TDHDHHQEF |
| 38038 | TDKPMMGGH |
| 38039 | TDPTRSTVP |
| 38040 | TDRVGMPPY |
| 38041 | VDGNEEEKP |
| 38042 | VDHDVDTMQ |
| 38043 | VDNHGFNDW |
| 38044 | VDWGVDPWH |
| 38045 | VDYCKEFVW |
| 38046 | WDKAQDVRM |
| 38047 | WDSHCQKIQ |
| 38048 | WDSPFNYSF |
| 38049 | WDSRYKCPI |
| 38050 | YDAEHQQLA |
| 38051 | YDDCTTNQG |
| 38052 | YDFPIMTCE |
| 38053 | YDPDKMKPG |
| 38054 | CDWMANVSV |
| 38055 | LDTIQINEV |
| 38056 | EQPHADQKI |
| 38057 | FAPSGKFRD |
| 38058 | FVPFHWWSH |
| 38059 | GCPPDPWHN |
| 38060 | GMPVMAFQT |
| 38061 | GPPECHMML |
| 38062 | GSPQWLEYT |
| 38063 | HSPLEFSSP |
| 38064 | IAPLQCPVS |
| 38065 | IFPMADCRV |
| 38066 | IQPYYWNDT |
| 38067 | KTPIKCEQH |
| 38068 | KVPPYRTCP |
| 38069 | KWPGMMGEV |
| 38070 | LEPNLMNTE |
| 38071 | LMPHRTWKC |
| 38072 | LNPHFVFGR |
| 38073 | LPPTCANGT |
| 38074 | LVPMAHRAC |
| 38075 | MAPQPMSWG |
| 38076 | MCPMFFWCE |
| 38077 | MLPYVCDPG |
| 38078 | MPPPNHLDV |
| 38079 | MTPSWIVED |
| 38080 | NCPAMPLKI |
| 38081 | NRPIRAWKR |
| 38082 | NTPERPHRK |
| 38083 | NTPITIQPL |
| 38084 | NYPSAHQCG |
| 38085 | QFPEIHRGE |
| 38086 | QMPTTTDRC |
| 38087 | QQPMIAEHV |
| 38088 | RNPSIQYWS |
| 38089 | RVPYVETYD |
| 38090 | SCPEMDLHR |
| 38091 | SNPIMDSDC |
| 38092 | SPPISYPRM |
| 38093 | SQPVMELSE |
| 38094 | SVPFTNMQS |
| 38095 | SVPMWWCAP |
| 38096 | TRPCICCDV |
| 38097 | TSPQNDHYS |
| 38098 | TTPFEKCCA |
| 38099 | TVPLQFQDS |
| 38100 | TVPRYSRNQ |
| 38101 | TYPRMITPA |
| 38102 | VGPCVRSDW |
| 38103 | VGPPYDTQD |
| 38104 | VLPFGAVGT |
| 38105 | VMPANWFFS |
| 38106 | VQPCMQCWC |
| 38107 | VRPMHMNCT |
| 38108 | VTPRFQWLT |
| 38109 | VYPTHNEVV |
| 38110 | WNPVQWTHS |
| 38111 | WTPVPSSSN |
| 38112 | WWPMRKWVS |
| 38113 | YLPYQSICG |
| 38114 | YNPQQRSLS |
| 38115 | IVPQQKIDE |
| 38116 | HGPFCDHAY |
| 38117 | KGPIFHSAY |
| 38118 | ENKCFASTT |
| 38119 | EQLCGKKKQ |
| 38120 | ERTCMPSAY |
| 38121 | ESDCFMWLG |
| 38122 | EYECLDCNI |
| 38123 | FCYCSHFPW |
| 38124 | FHMCSCTRY |
| 38125 | FSSCFNAVE |
| 38126 | FWRCDPNKV |
| 38127 | FYNCCDNPL |
| 38128 | GCMCEGAEF |
| 38129 | GGFCPCDQK |
| 38130 | GGSCSWDKP |
| 38131 | GIICYDATN |
| 38132 | HRGCCESGR |
| 38133 | HSFCVEGIP |
| 38134 | IHFCMCCDF |
| 38135 | IHRCIAEAA |
| 38136 | IHTCPKNTQ |
| 38137 | ILDCHKHYS |
| 38138 | ITECHPRKT |
| 38139 | ITYCNVVYW |
| 38140 | IVVCMHCEQ |
| 38141 | KGMCLSQGD |
| 38142 | KGVCKGVLP |
| 38143 | KYECPFYYV |
| 38144 | LHDCQMWGD |
| 38145 | LHSCDTPWT |
| 38146 | LKECCFEDA |
| 38147 | LNECYYNRP |
| 38148 | LPDCTDYRK |
| 38149 | LPRCCTINY |
| 38150 | LVGCSYCQS |
| 38151 | MAGCPPHDS |
| 38152 | MASCIPQTS |
| 38153 | MFACTQFFG |
| 38154 | MGDCHRSPD |
| 38155 | MMRCNVDFE |
| 38156 | MMSCIAAVQ |
| 38157 | MVSCRSAAC |
| 38158 | NASCHWHFD |
| 38159 | NGLCVESTA |
| 38160 | NPACNKMPT |
| 38161 | NQACNGWHK |
| 38162 | NSHCTWRKF |
| 38163 | NVECLPMLP |
| 38164 | QAMCCSYPR |
| 38165 | QAMCVHWHM |
| 38166 | QCYCAKYWS |
| 38167 | QGDCYEMWE |
| 38168 | QMTCPSDLW |
| 38169 | QQICPVGEF |
| 38170 | QYACHQIGA |
| 38171 | RGACDEFET |
| 38172 | RLMCYFRTW |
| 38173 | RPACTVPGK |
| 38174 | SGDCRDWHD |
| 38175 | SGTCYHYAL |
| 38176 | STGCGLMWG |
| 38177 | SVYCEEFYT |
| 38178 | TIMCVASLA |
| 38179 | TLYCYDDYH |
| 38180 | TRICCKNRQ |
| 38181 | VKQCTKFDE |
| 38182 | VWCCSVNPG |
| 38183 | WRNCMQDSK |
| 38184 | WSICHKDSY |
| 38185 | QNWCGKNLE |
| 38186 | SQDCGRPES |
| 38187 | KVGCFVDVW |
| 38188 | FCKCFMKHC |
| 38189 | ESQRDDGWL |
| 38190 | ESRADIEVA |
| 38191 | ETCIDMQGK |
| 38192 | ETHTDYQGP |
| 38193 | ETNPDWEGR |
| 38194 | FCTSDQKAL |
| 38195 | FGDHDNMPE |
| 38196 | FRHHDVNNH |
| 38197 | GEYYDEVNH |
| 38198 | GMEPDTQLY |
| 38199 | GMVPDLLPH |
| 38200 | GVNMDMEVS |
| 38201 | HGELDCASC |
| 38202 | HHFTDVDAH |
| 38203 | HHHADFHLA |
| 38204 | HMHRDMHEE |
| 38205 | HPNRDSAHM |
| 38206 | HRDGDILIE |
| 38207 | HYTSDWFQA |
| 38208 | IAKEDICQP |
| 38209 | ICVNDNFHN |
| 38210 | ISAPDNQLS |
| 38211 | IVTADNTME |
| 38212 | KANWDVNHS |
| 38213 | KENGDSITL |
| 38214 | KVDPDCHMF |
| 38215 | KVLQDQFTI |
| 38216 | KVVTDRKWD |
| 38217 | KYEPDGFIP |
| 38218 | LVGLDLSCH |
| 38219 | MHYSDGFVS |
| 38220 | MIEYDVGYK |
| 38221 | MLKTDCPLH |
| 38222 | MPCMDRRRP |
| 38223 | MRCADMLRY |
| 38224 | MTESDDRCV |
| 38225 | NCGLDITMR |
| 38226 | NIQADMSQQ |
| 38227 | NTVMDRMNF |
| 38228 | QCDSDTQSR |
| 38229 | QTRKDGESH |
| 38230 | QTRWDLIGL |
| 38231 | QVCIDWSGG |
| 38232 | QVNEDDAAK |
| 38233 | REYMDDEKQ |
| 38234 | RHHIDIPTY |
| 38235 | RVLTDERMD |
| 38236 | SAREDDNPC |
| 38237 | SCRIDWTDW |
| 38238 | SFNYDMVSR |
| 38239 | SGRGDVNGA |
| 38240 | SMQVDVLQR |
| 38241 | SNQWDWHIF |
| 38242 | SVMLDEWIN |
| 38243 | SWCIDCEAD |
| 38244 | TIAVDGHHS |
| 38245 | TILPDSAEF |
| 38246 | TIVYDNWSR |
| 38247 | TTTADKPAS |
| 38248 | TTVLDHGLQ |
| 38249 | TWKEDWALL |
| 38250 | TYKRDIMKT |
| 38251 | VILKDRVNW |
| 38252 | VKKSDAKQC |
| 38253 | VQDGDYSPS |
| 38254 | VSMEDHCKH |
| 38255 | WEKFDRPWR |
| 38256 | WPSADCDIP |
| 38257 | YMSRDQIKS |
| 38258 | YPTRDNLFM |
| 38259 | YWNFDDYQQ |
| 38260 | YYTGDLTYK |
| 38261 | SVKQDCVED |
| 38262 | TVREDCCIN |
| 38263 | YFAADEQWG |
| 38264 | NQIKDVAAS |
| 38265 | DARFDQWHH |
| 38266 | ALTHDDGRT |
| 38267 | FAYFQKDLF |
| 38268 | FNADHKCPS |
| 38269 | GRDALKNGD |
| 38270 | GVATLKWEQ |
| 38271 | GVCSKKSGA |
| 38272 | HFAEIKYET |
| 38273 | HMRMCKATW |
| 38274 | HPRSTKWCQ |
| 38275 | IEEHLKVCY |
| 38276 | IVATPKQYG |
| 38277 | KLNHVKRTS |
| 38278 | MCNYHKLNK |
| 38279 | MGGPYKLMC |
| 38280 | MGNIVKPAD |
| 38281 | MIDRMKESY |
| 38282 | MRTNYKLEM |
| 38283 | NFWLAKPFK |
| 38284 | NLHGTKFGY |
| 38285 | QMMQTKLHD |
| 38286 | QMTIPKWGY |
| 38287 | QSCWTKCEA |
| 38288 | SGILSKDWK |
| 38289 | SIDEPKFTV |
| 38290 | TEHENKCVG |
| 38291 | TGLLRKEQS |
| 38292 | TKNEFKTND |
| 38293 | TMAQCKYPH |
| 38294 | TMFSHKPSD |
| 38295 | VMCVPKYKD |
| 38296 | VNEEMKFQP |
| 38297 | WNGHHKDML |
| 38298 | YWFLHKGCN |
| 38299 | EVARTKFGG |
| 38300 | QMSWGKYHQ |
| 38301 | SYYQSKNTD |
| 38302 | LWFFMKFAK |
| 38303 | GACLWKRHL |
| 38304 | THARFKMKS |
| 38305 | LMMMCKQQY |
| 38306 | ESSWLYPHN |
| 38307 | FSITEEPFW |
| 38308 | GVVDYWPTF |
| 38309 | HHTLNYPNE |
| 38310 | HWESFIPRV |
| 38311 | IGGWIWPGP |
| 38312 | IVSELCPSF |
| 38313 | KKQMPFPRD |
| 38314 | LGRAPCPCW |
| 38315 | LSNVPMPAQ |
| 38316 | MAAEAYPCI |
| 38317 | MHAEFGPLP |
| 38318 | MKFQSGPKS |
| 38319 | NQFRAEPIS |
| 38320 | NVTNFDPHP |
| 38321 | QAAPGPPYE |
| 38322 | QPNGWAPPC |
| 38323 | QQFQTDPQT |
| 38324 | QVEIQEPAI |
| 38325 | RMLVRVPDG |
| 38326 | RPDMLTPYR |
| 38327 | SQCTPFPFN |
| 38328 | SRDRRQPKK |
| 38329 | SSFHEDPCN |
| 38330 | THHGLQPND |
| 38331 | TKVVIEPNC |
| 38332 | TLYGHDPAH |
| 38333 | TMFSFQPVE |
| 38334 | VINNRVPPT |
| 38335 | VQNYADPTG |
| 38336 | YGTIPVPIT |
| 38337 | YIKAVTPPF |
| 38338 | IREPTEPYC |
| 38339 | IFQLSRPAW |
| 38340 | ISGWRMPCD |
| 38341 | QAEVFAPYH |
| 38342 | DAMWKFPST |
| 38343 | TVNPCIPDN |
| 38344 | KCLQMCPNG |
| 38345 | FCNTHCGKP |
| 38346 | GLDWVSVKA |
| 38347 | HGNAETHKD |
| 38348 | HITNPTSKP |
| 38349 | HPEWKMSKC |
| 38350 | IAKQEHVKS |
| 38351 | IVDALHHKW |
| 38352 | IWTHLCVKH |
| 38353 | LFIQYYHKS |
| 38354 | LQNDWDAKL |
| 38355 | LVIPRDTKK |
| 38356 | LYHETHKKC |
| 38357 | MAIPTGVKE |
| 38358 | MAVEYDRKG |
| 38359 | MMNWWMYKA |
| 38360 | MVTQTYFKN |
| 38361 | NCMAVWAKN |
| 38362 | NLDQWNVKV |
| 38363 | NTEGCRVKK |
| 38364 | QIGTPSQKA |
| 38365 | QIQMTDDKQ |
| 38366 | QSAQWNVKA |
| 38367 | RTFNRATKT |
| 38368 | SLVHFHAKT |
| 38369 | TFGFFCTKG |
| 38370 | TFSTGLSKK |
| 38371 | TKGFMQKKE |
| 38372 | TMDEHHMKI |
| 38373 | TSVDLQMKA |
| 38374 | VEMWVWEKK |
| 38375 | VKQFNAHKG |
| 38376 | VNLTTSVKF |
| 38377 | WCYQCILKF |
| 38378 | WSMQLSHKM |
| 38379 | YAWTTVCKT |
| 38380 | NMVWKCRKS |
| 38381 | VMWTWESKY |
| 38382 | EVLDSMSEV |
| 38383 | EVTHPIGAV |
| 38384 | FASRCPRTV |
| 38385 | FFFTRWMRV |
| 38386 | FGVEAWLPV |
| 38387 | FHGERHQDV |
| 38388 | FVRVQQRAV |
| 38389 | HACIHTDEV |
| 38390 | HLSEFNMQV |
| 38391 | HTFYCTCRV |
| 38392 | HVSIATGSV |
| 38393 | HYDFQLTQV |
| 38394 | ICVNKQQSV |
| 38395 | IFHSHATCV |
| 38396 | IFSYSQRMV |
| 38397 | IRYPYGKMV |
| 38398 | KSSEIVKWV |
| 38399 | KSYQRTFLV |
| 38400 | KYTGGIVVV |
| 38401 | LTNWNGMHV |
| 38402 | MIETTGQQV |
| 38403 | MMGPPQHVV |
| 38404 | MPKWGREPV |
| 38405 | MVEATDWWV |
| 38406 | MWTTETMGV |
| 38407 | MWVMGPLCV |
| 38408 | NHCMWEGYV |
| 38409 | NHHPFMYYV |
| 38410 | NIMLGQNSV |
| 38411 | NWVHPITRV |
| 38412 | QGCHFNMSV |
| 38413 | QVVKFEIPV |
| 38414 | RRQWQDFMV |
| 38415 | SAGYMCYDV |
| 38416 | SGDGFHYGV |
| 38417 | SIMHLCEHV |
| 38418 | SNCSVVKCV |
| 38419 | SPVILNADV |
| 38420 | STRMCQMLV |
| 38421 | SWQQSPYFV |
| 38422 | TARYYDGPV |
| 38423 | TCGYFTRDV |
| 38424 | TWASRRKCV |
| 38425 | VKCLGAMNV |
| 38426 | VNCKHMNNV |
| 38427 | VNYSWNIQV |
| 38428 | VVMPFWSAV |
| 38429 | WTVELANGV |
| 38430 | WVQPRMSIV |
| 38431 | YHEHAVESV |
| 38432 | YLEANMCHV |
| 38433 | YQHWTTMGV |
| 38434 | YTTFYRFQV |
| 38435 | QVMLHHVWV |
| 38436 | QNLMYCSSV |
| 38437 | TCGQYVNGV |

### Example 7

AAV5 Variants with Tissue Tropism in Adrenal Gland

This example describes engineered AAV5 variants with tissue tropism in adrenal gland that were discovered using the methods and systems described in EXAMPLE 1-EXAMPLE 2.

CNS, liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive adrenal gland tropism. The results are shown in FIG. 18K which shows the positional frequency of each amino acid in the 581 to 589 region of AAV5 VP1 in adrenal gland tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in adrenal gland over the frequency of that given amino acid residue occurring at the specified position in variants identified in all other harvested tissues was analyzed to identify a set of sequence rules for capsids that preferentially target adrenal gland tissue. With reference to TABLE 13 below, and SEQ ID NO: 2 (AAV5 VP1), the following amino acids can, thus, be independently mutated, in any combination, at any one or more positions Xaa1-Xaa9, to provide a VP1 capsid with enhanced adrenal gland tropism as compared wildtype AAV5 VP1 capsid (SEQ ID NO: 1), where adrenal gland tropism here refers to properties that are preferred for adrenal gland transduction over properties that are preferred for transduction of all other harvested tissues.

TABLE 13
Adrenal Gland Tropism Rules

Xaa1 is selected from A, C, K, Q, R, S, or T
Xaa1 is selected from C, K, or R
Xaa1 is C
Xaa2 is selected from A, C, I, S, T, or V
Xaa2 is selected from A, V, or T
Xaa2 is V
Xaa3 is selected from A, F, G, K, M, Q, R, T, or V
Xaa3 is selected from A, G, or M
Xaa3 is M
Xaa4 is selected from A, K, M, Q, R, or V
Xaa4 is selected from A, R, or K
Xaa4 is K
Xaa5 is selected from F, I, L, M, R, T, V, or Y
Xaa5 is selected from R, V, or Y
Xaa5 is V
Xaa6 is selected from G, H, M, N, R, or S
Xaa6 is selected from H or N
Xaa6 is N
Xaa7 is selected from A, H, K, Q, R, S or V
Xaa7 is selected from H, Q, or V
Xaa7 is H
Xaa8 is selected from A, G, H, M, Q, or S
Xaa8 is selected from A, G, M, or S
Xaa8 is S
Xaa9 is selected from A, E, N, P, R, S, or Y
Xaa9 is selected from P or E
Xaa9 is P

TABLE 14 below provides sequences of the 581 to 589 region for variant AAV VP1 capsid polypeptides that were found in adrenal gland tissue and comport to one or more of the rules provided in TABLE 13. The present disclosure, thus, provides for rAAVs composed of engineered AAV5 VP1 capsid polypeptides having a SEQ ID NO: 2, wherein the Xaa1 to Xaa9 region is any one of SEQ ID NO: 1118-SEQ ID NO: 2117, as disclosed in TABLE 14. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 14
Sequences of the 581 to 589 Region in AAV5 VP1
Capsid Polypeptide that
Drive Adrenal Gland Tissue Tropism

| SEQ ID | 581-589 |
|---|---|
| NO | Sequence |
| 1118 | CAAQYWLTP |
| 1119 | CARFVERWP |
| 1120 | CCAKFQMCT |
| 1121 | CEADGNWMR |
| 1122 | CIDMVQSWS |
| 1123 | CMLLAEKMF |
| 1124 | CMYPKSTDR |
| 1125 | CSLPYVREG |
| 1126 | CSVKMPGFR |
| 1127 | CTANVWTAQ |
| 1128 | CTVVMMRQP |
| 1129 | CVIPLRMYM |
| 1130 | CVIQTSRGI |
| 1131 | CAAYRGLLE |
| 1132 | CAFQPVRWF |
| 1133 | CAFQRMMYQ |
| 1134 | CAHQPQRDR |
| 1135 | CANYAFMTE |
| 1136 | CAPLVNSFC |
| 1137 | CARMFAAQW |
| 1138 | CCHFQHKAG |
| 1139 | CCLQPELTM |
| 1140 | CCRPFVLHQ |
| 1141 | CDVKCKKED |
| 1142 | CEMIANTNN |
| 1143 | CFNIIQRYD |
| 1144 | CFRQVQAMS |
| 1145 | CGDWLWRNL |
| 1146 | CHGHRPFDA |
| 1147 | CHIDYDLGF |
| 1148 | CHMKFNKFG |
| 1149 | CIGKCWPPM |
| 1150 | CIGRVNGTR |
| 1151 | CIKLTMDDR |
| 1152 | CIKSKFSWQ |
| 1153 | CINEKQIRW |
| 1154 | CIVKFSNTQ |
| 1155 | CKMATNGMT |
| 1156 | CKMYMHESR |
| 1157 | CLMALKRSG |
| 1158 | CLSSYSRYQ |
| 1159 | CLVKYPQFG |
| 1160 | CMYFIAEWH |
| 1161 | CPDEPRWFP |
| 1162 | CPFKVFAHG |
| 1163 | CPMIDEFPT |
| 1164 | CPTWSCQQW |
| 1165 | CPWQIKVAA |
| 1166 | CQMEQHYSP |
| 1167 | CSAPLRHWY |
| 1168 | CSIFGPTYP |
| 1169 | CSKFIVDWE |
| 1170 | CSLRYFMAR |
| 1171 | CSRTMEVGL |
| 1172 | CSSVRWMSP |
| 1173 | CSSYSFQHS |
| 1174 | CTFMANWGF |
| 1175 | CTGQRGFAN |
| 1176 | CTMDQWQTA |
| 1177 | CTTHFYRAR |
| 1178 | CVAPVRLQD |
| 1179 | CVAYRLVNP |
| 1180 | CVFMRLTSG |
| 1181 | CVFVRNNMM |
| 1182 | CVGSLMRIQ |
| 1183 | CVHLTSSSA |
| 1184 | CVKQYMQPP |
| 1185 | CVRHTENWC |
| 1186 | CVTTRHKQC |
| 1187 | CVWSAPVEC |
| 1188 | CVYQSRFSC |
| 1189 | CWNENTYEA |
| 1190 | CWNGIQRQA |
| 1191 | CYTRFHYSE |
| 1192 | CYVFRHHYC |
| 1193 | HVASTSYVT |
| 1194 | HVFDTDRWM |
| 1195 | HVFHLDEAN |
| 1196 | HVKDTDVQI |
| 1197 | HVQARQNPG |
| 1198 | HVSDKNHAK |
| 1199 | HVVPQNWNW |
| 1200 | HVVQRLTIP |
| 1201 | IVEARLEFN |
| 1202 | IVEDFEIVI |
| 1203 | IVFYRFEFV |
| 1204 | IVLPTMLLA |
| 1205 | IVVIAKMTT |
| 1206 | IVVSTWHEQ |
| 1207 | IVWLQRVGD |
| 1208 | KVDHTINQM |
| 1209 | KVDTEKNMM |
| 1210 | KVTGHEQDV |
| 1211 | KVVLWEHFQ |
| 1212 | LVCTHTKAA |
| 1213 | LVDDDYQMD |
| 1214 | LVEIPCSNW |
| 1215 | LVHSCQHTE |
| 1216 | LVLQSFHHS |
| 1217 | LVNCYTYRA |
| 1218 | LVQVMDMKP |
| 1219 | LVVIFEGRH |
| 1220 | LVWMDPHWG |
| 1221 | MVAAKEKHD |
| 1222 | MVAHNGLGF |
| 1223 | MVCMLVHHC |
| 1224 | MVDHSYQQE |
| 1225 | MVEVIEGGQ |
| 1226 | MVHGNHECW |
| 1227 | MVNPMHCCN |
| 1228 | MVRKYEKMH |
| 1229 | MVSCNQHDS |
| 1230 | MVSHVVASE |
| 1231 | MVSVDQMSV |
| 1232 | MVWSRADWS |
| 1233 | NVAIILSTD |
| 1234 | NVAVEHNMG |
| 1235 | NVEDTRKGS |
| 1236 | NVFLAHQRA |
| 1237 | NVHNSGRFD |
| 1238 | NVIGTENMS |
| 1239 | NVKSEHRES |
| 1240 | NVLLEKAFW |
| 1241 | NVNRGHLLE |
| 1242 | NVNWQARND |
| 1243 | NVTHKQGCS |
| 1244 | NVYPDSMHG |
| 1245 | PVNFFEHPM |
| 1246 | QVDIDTQNA |
| 1247 | QVDPHGNMH |
| 1248 | QVDRWCTAA |
| 1249 | QVEYAHSRA |
| 1250 | QVFMQHDIE |
| 1251 | QVGNPLKEF |
| 1252 | QVIEWGRSG |
| 1253 | QVIIRKWFA |
| 1254 | QVLMAQFSR |
| 1255 | QVQKCMQYV |
| 1256 | QVQKGACPS |
| 1257 | QVQSGPYWG |
| 1258 | QVQTENMCD |
| 1259 | QVRCGIKTG |
| 1260 | QVTFHAYVC |
| 1261 | QVTIQNDYD |
| 1262 | QVWNPHLCM |
| 1263 | RVATEHFRE |
| 1264 | RVDNPFVSY |
| 1265 | RVSECTENY |
| 1266 | RVTEEVGSR |
| 1267 | SVDQHDHLL |
| 1268 | SVERYPLTA |
| 1269 | SVFDVVHEE |
| 1270 | SVHVRIVEP |
| 1271 | SVKQHHVDW |
| 1272 | SVLGMPAYR |
| 1273 | SVMDANTWR |
| 1274 | SVNKDVRHC |
| 1275 | SVNSYMDSV |
| 1276 | SVPRKSKCS |
| 1277 | SVRSYHSQP |
| 1278 | SVTQPLYCQ |
| 1279 | SVVKRNDDV |
| 1280 | TVAHFLNGG |
| 1281 | TVALSEQYR |
| 1282 | TVAVRYETL |
| 1283 | TVCSTIVDW |
| 1284 | TVDWRWGDF |
| 1285 | TVEHKMDAP |
| 1286 | TVISDYEDC |
| 1287 | TVKMLQHWT |
| 1288 | TVMKEQANM |
| 1289 | TVNDHGPCI |
| 1290 | TVNKWHQTA |
| 1291 | TVNQCMTHC |
| 1292 | TVYFVWDSQ |
| 1293 | VVGATATGM |
| 1294 | VVGPPGHHY |
| 1295 | VVMFWQEGD |
| 1296 | VVMQQYRSH |
| 1297 | VVNKDKWRI |
| 1298 | VVQWPTSLL |
| 1299 | VVSAAKSSG |
| 1300 | VVSHKQRCS |
| 1301 | VVSKYQIVP |
| 1302 | VVTCRHQGS |
| 1303 | VVVPMSNCE |
| 1304 | WVMCISGHY |
| 1305 | YVMFNTWRE |
| 1306 | YVTMSDERP |
| 1307 | AVGFMECAN |
| 1308 | DVKDRNELH |
| 1309 | EVSAEEGQN |
| 1310 | GVMPRWGLF |
| 1311 | GVVNLVKMQ |
| 1312 | IVMYDHFNQ |
| 1313 | KVILDSFRQ |
| 1314 | KVMPEPVTE |
| 1315 | LVHLFQYHL |
| 1316 | QVVACMHHT |
| 1317 | RVDYSAIHM |
| 1318 | SVIMHHDQH |
| 1319 | VVEMNGDDD |
| 1320 | VVEYAPWAW |
| 1321 | AVAKGVGVT |
| 1322 | AVMATGVTQ |
| 1323 | AVSGNWMMT |
| 1324 | AVSKSGCGM |
| 1325 | DVAAVQVLE |
| 1326 | DVASYEQAY |
| 1327 | DVEWQQSWT |
| 1328 | DVGGLNREY |
| 1329 | DVHEVMSCG |
| 1330 | DVHICTVHI |
| 1331 | DVKGMLDGF |
| 1332 | DVKMQQKYF |
| 1333 | DVMGHAKLA |
| 1334 | DVRAYLNKE |
| 1335 | DVRYNQNAW |
| 1336 | DVSFMGWDC |
| 1337 | DVTHHFAKQ |
| 1338 | EVAHFLANE |
| 1339 | EVDNMQMGE |
| 1340 | EVGVSAPMM |
| 1341 | EVSGMFHPA |
| 1342 | EVTTMLKAC |
| 1343 | EVWINMAPE |
| 1344 | EVYMEPANW |
| 1345 | GVFFRYEHG |
| 1346 | GVFSTGVVT |
| 1347 | GVGVGLGTQ |
| 1348 | GVHQESGQN |
| 1349 | GVNKLSYEN |
| 1350 | GVRIPMAAD |
| 1351 | GVSQFMAET |
| 1352 | HVGAHTRSD |
| 1353 | HVGKMPGGS |
| 1354 | HVNSFQPDR |
| 1355 | IVCTTFEDS |
| 1356 | IVDQTCMHA |
| 1357 | IVGIWQWMG |
| 1358 | IVKNLEQYM |
| 1359 | IVKTLICYN |
| 1360 | KVDVITVKD |
| 1361 | KVGAHNDEV |
| 1362 | KVTWLDLED |
| 1363 | LVEAVMNAT |
| 1364 | LVKACTDQH |
| 1365 | LVKQYGLIH |
| 1366 | LVLMDRQAY |
| 1367 | LVSQTPRKV |
| 1368 | LVVDHAEWS |
| 1369 | MVALTDQLG |
| 1370 | MVDSNACLC |
| 1371 | MVHPSQNVD |
| 1372 | MVKEAPYYR |
| 1373 | MVSEYATNA |
| 1374 | MVTGISKSS |
| 1375 | NVESNYNLI |
| 1376 | NVEWGTVQS |
| 1377 | NVFMTSFQC |
| 1378 | NVFQPEDHC |
| 1379 | NVTWEDVQE |
| 1380 | PVAAAEPRN |
| 1381 | QVAEPGGRF |
| 1382 | QVDSWPVLS |
| 1383 | QVGCTVGPG |
| 1384 | RVQHSGAHW |
| 1385 | RVTNTWECM |
| 1386 | RVYTTGKCS |
| 1387 | SVHHSRWGY |
| 1388 | SVNQAMARK |
| 1389 | SVSVMTYSE |
| 1390 | TVAMDPDYA |
| 1391 | TVATEPAQQ |
| 1392 | TVDFFVMDC |
| 1393 | TVDHCVGTV |
| 1394 | TVGLNERNG |
| 1395 | TVHWWECVQ |
| 1396 | TVQTNFTSK |
| 1397 | TVTQEWMMK |
| 1398 | TVVGLITNA |
| 1399 | VVEKRMDRS |
| 1400 | VVHGYVCKQ |
| 1401 | VVHPLAYCT |
| 1402 | VVNKQQISE |
| 1403 | VVTSPDAFQ |
| 1404 | VVVHTQCNP |
| 1405 | WVTPTNHKP |
| 1406 | YVAEYGLGA |
| 1407 | YVEERGSQR |
| 1408 | YVIVHMKGM |
| 1409 | YVTPNIAVH |
| 1410 | HCMRQSESG |
| 1411 | HSMPEAVAY |
| 1412 | HTMEILPGL |
| 1413 | HTMHDSCFN |
| 1414 | HTMLWAKDE |
| 1415 | ICMSYLSWD |
| 1416 | IPMFSIHGN |
| 1417 | KAMLEGPYL |
| 1418 | KNMINEAAP |
| 1419 | KSMWEHAIF |
| 1420 | KTMEDVIHA |
| 1421 | KYMSYERES |
| 1422 | LAMTLAVGQ |
| 1423 | LCMDYALVD |
| 1424 | LCMLLASQQ |
| 1425 | LSMCFRDDY |
| 1426 | MCMVERRGS |
| 1427 | MFMSQHVED |
| 1428 | MNMHTDCTT |
| 1429 | NAMWTTEYE |
| 1430 | NEMKFSYSH |
| 1431 | NIMNWQNGD |
| 1432 | NLMPTNHAM |
| 1433 | NSMQHCVCP |
| 1434 | NTMHPVGMM |
| 1435 | NTMMVGRCE |
| 1436 | PIMFEGECK |
| 1437 | QCMMKSSYE |
| 1438 | QFMPYLGFA |
| 1439 | QGMENQWHQ |
| 1440 | QHMYANCLP |
| 1441 | QKMTRQHWG |
| 1442 | QSMEQEFVT |
| 1443 | QSMMALKDY |
| 1444 | QSMMTDRTY |
| 1445 | REMTKVGGR |
| 1446 | RGMYRCGWD |
| 1447 | RNMYISQWE |
| 1448 | RSMGNEQDP |
| 1449 | SEMKLVNLP |
| 1450 | SEMVKHNFE |
| 1451 | SGMKQVGAA |
| 1452 | SGMLVGIYN |
| 1453 | SHMVSNSAV |
| 1454 | SSMSKHFVA |
| 1455 | STMPGTDQK |
| 1456 | TAMHQTMLN |
| 1457 | TAMMSGHLV |
| 1458 | TAMVVEYDQ |
| 1459 | TCMKNINYQ |
| 1460 | TCMLGAEAA |
| 1461 | TCMQLATMQ |
| 1462 | TCMTLRHIG |
| 1463 | TCMYYWKQP |
| 1464 | THMLCPRMD |
| 1465 | TNMSYQRSD |
| 1466 | TRMPVNQSC |
| 1467 | TWMEWHANA |
| 1468 | TYMGTHNHS |
| 1469 | VFMPDVYDK |
| 1470 | VKMPYHYEG |
| 1471 | VQMQEVPHY |
| 1472 | WFMHYSWKH |
| 1473 | WKMEGDCFK |
| 1474 | EIMCAYVKA |
| 1475 | GAMPNPYHE |
| 1476 | GTMWSTKSC |
| 1477 | HPMFMIPGY |
| 1478 | HYMLKEYFA |
| 1479 | IHMNTIVGE |
| 1480 | IMMAQWFAH |
| 1481 | PSMPNIKPS |
| 1482 | PYMQYWYWE |
| 1483 | REMLRDKMP |
| 1484 | SAMQVMTMC |
| 1485 | TAMSICMYG |
| 1486 | TFMDKGLIR |
| 1487 | ANMFRNGMQ |
| 1488 | ASMFWNYQV |
| 1489 | DRMVIKSAE |
| 1490 | EAMRAMTPI |
| 1491 | EAMYWQCAI |
| 1492 | EIMWEGVGS |
| 1493 | ENMHKEYVQ |
| 1494 | EPMEHYLRS |
| 1495 | GAMTMCCEW |
| 1496 | GKMMGLHSH |
| 1497 | ILMEYDHDK |
| 1498 | IMMHRVMSD |
| 1499 | KAMEVHFQK |
| 1500 | KAMGLPSWY |
| 1501 | KSMWEDRIC |
| 1502 | LAMPWDTLQ |
| 1503 | LCMLRMMGH |
| 1504 | LIMAHWLDM |
| 1505 | LIMDSKMGQ |
| 1506 | LLMPVLHQM |
| 1507 | MGMKREGSG |
| 1508 | MQMSKNKHP |
| 1509 | MRMIHWMHC |
| 1510 | NKMEWHKYC |
| 1511 | NWMQNTEGH |
| 1512 | PEMSFEFTT |
| 1513 | PGMNMVRKE |
| 1514 | PTMWWYLLV |
| 1515 | QCMIVSGMT |
| 1516 | QGMHAMFYA |
| 1517 | QMMQLYCGA |
| 1518 | SAMVHSNKS |
| 1519 | SHMSYLMSH |
| 1520 | TCMPNIQCP |
| 1521 | TFMHQFGTY |
| 1522 | TQMMTPVGA |
| 1523 | TWMADWHQE |
| 1524 | VAMQSMQEH |
| 1525 | VAMQTHQCY |
| 1526 | WWMHGRVGR |
| 1527 | YCMLKHKAG |
| 1528 | YCMWQQTSN |
| 1529 | YKMDHNNTY |
| 1530 | YSMTWNQQT |
| 1531 | HTGKADVSV |
| 1532 | IMEKLRRSS |
| 1533 | IPFKAVMVG |
| 1534 | IRVKPGCRV |
| 1535 | ITGKYPMTA |
| 1536 | KLEKTVVYS |
| 1537 | LDIKMDFLC |
| 1538 | LMHKDQVSP |
| 1539 | LNVKTGNAW |
| 1540 | LTTKQPDMP |
| 1541 | MASKLHMAV |
| 1542 | NSLKYEFSE |
| 1543 | NTGKTGQFN |
| 1544 | PCAKSYDQS |
| 1545 | PKDKGVICE |
| 1546 | QEVKGQWWP |
| 1547 | QIFKQTEVQ |
| 1548 | QITKPILDR |
| 1549 | QLAKAMTQS |
| 1550 | QPVKYPQTN |
| 1551 | RISKDGSDR |
| 1552 | RIVKRYHNG |
| 1553 | SLCKMLTAK |
| 1554 | SYKKPVKYM |
| 1555 | SYNKVMTQH |
| 1556 | TACKTEGGP |
| 1557 | TCVKTTHGA |
| 1558 | TDGKTRMQQ |
| 1559 | TETKWQHLE |
| 1560 | TIHKYCHHQ |
| 1561 | TSTKHHWWC |
| 1562 | TSVKKPHLA |
| 1563 | TTLKCTNTW |
| 1564 | TWIKPHQQL |
| 1565 | VCEKSNDYA |
| 1566 | VCFKMLLGN |
| 1567 | VECKTMAHQ |
| 1568 | VNAKWQTAS |
| 1569 | VSCKIYHYA |
| 1570 | VSGKECSED |
| 1571 | VTYKYYCDF |
| 1572 | WYQKQAEHG |
| 1573 | YCSKSVRCC |
| 1574 | NFSKCAKYD |
| 1575 | VCTKGFLNS |
| 1576 | AAHKEMKDT |
| 1577 | ACGKMHGPN |
| 1578 | AFLKQSIIH |
| 1579 | AGSKRFDWF |
| 1580 | AILKMTKVL |
| 1581 | DTGKFMCKW |
| 1582 | EHQKNINMH |
| 1583 | ESEKVWMMT |
| 1584 | EWRKKKKRT |
| 1585 | FCRKIEQAN |
| 1586 | FICKTLHQH |
| 1587 | GCDKTRFEN |
| 1588 | GILKFRDFM |
| 1589 | ICWKFLVSN |
| 1590 | IEVKYWNHH |
| 1591 | IWYKWWVMF |
| 1592 | KCDKPMLKP |
| 1593 | KMEKVERMP |
| 1594 | LDLKHNMCD |
| 1595 | MCIKCDAND |
| 1596 | NCNKCKNRT |
| 1597 | NQCKINVGT |
| 1598 | QQLKTNINF |
| 1599 | RIEKLQCDY |
| 1600 | STLKTYAYN |
| 1601 | TGTKITMQT |
| 1602 | TIHKDGEFS |
| 1603 | VLQKFGHSF |
| 1604 | VTGKHIAQN |
| 1605 | YCYKTAVAA |
| 1606 | HCALVNRLR |
| 1607 | HFAMVWAGE |
| 1608 | HIGYVEAMR |
| 1609 | HTKPVNAQP |
| 1610 | IHQYVNFVH |
| 1611 | INNTVAHQE |
| 1612 | KALMVQQFD |
| 1613 | KCLMVDWGV |
| 1614 | KHGQVSIHD |
| 1615 | KMDRVGYSA |
| 1616 | KWNNVENNE |
| 1617 | KYSPVEWMY |
| 1618 | LGQHVQCWQ |
| 1619 | LKSWVCATS |
| 1620 | LMKCVMCRD |
| 1621 | MASPVNSEF |
| 1622 | MCLFVRVSQ |
| 1623 | MHGMVHAFE |
| 1624 | MLIHVNHKS |
| 1625 | MQDQVCKIG |
| 1626 | MQQPVNKHM |
| 1627 | MRQTVDRPE |
| 1628 | MYHAVQCMP |
| 1629 | MYYQVSKQD |
| 1630 | NAENVDNGV |
| 1631 | NFEFVERYR |
| 1632 | NGHEVPVEE |
| 1633 | NKQVVQMTA |
| 1634 | NLPLVHSLE |
| 1635 | NLQRVEMGV |
| 1636 | NMQNVARCY |
| 1637 | NSWDVGSFN |
| 1638 | NTYPVLSDW |
| 1639 | PEYFVVYMY |
| 1640 | PMVSVSGYY |
| 1641 | QGGAVMHEE |
| 1642 | QIQPVNALF |
| 1643 | QQLSVHMWH |
| 1644 | QSGWVQIAA |
| 1645 | QWQYVMTDK |
| 1646 | RCWSVEASQ |
| 1647 | RDQRVLKVM |
| 1648 | RTTGVGDIS |
| 1649 | SAWCVPGQL |
| 1650 | SCHLVIKHH |
| 1651 | SFAPVAHIE |
| 1652 | SGRHVLIEI |
| 1653 | SITAVWSDE |
| 1654 | SQNMVDSQC |
| 1655 | SSCIVNIAS |
| 1656 | SSNLVGAGE |
| 1657 | STELVKYID |
| 1658 | TMHPVCCPM |
| 1659 | TMREVQCEW |
| 1660 | TPAHVCRGY |
| 1661 | VNDMVLAVV |
| 1662 | VSFCVRKQS |
| 1663 | YALTVPHSE |
| 1664 | YTPNVHYMV |
| 1665 | ECANVIKFN |
| 1666 | GACTVQNMT |
| 1667 | HIEWVSMHR |
| 1668 | MGQNVNHAG |
| 1669 | QAAFVNYGP |
| 1670 | RGFHVIVDQ |
| 1671 | RYDLVSYHQ |
| 1672 | VFDTVFAYC |
| 1673 | VKKYVDVHF |
| 1674 | VQSAVSMCS |
| 1675 | APVFVEQMG |
| 1676 | ASTAVLVQR |
| 1677 | DAGFVREPV |
| 1678 | EAVPVESGD |
| 1679 | EEKEVICMC |
| 1680 | EFEQVGFMM |
| 1681 | EGTMVKQGE |
| 1682 | EHNCVYLTE |
| 1683 | EMHYVCCQT |
| 1684 | EPFRVTQCG |
| 1685 | FASIVFGGE |
| 1686 | FEYMVLNWH |
| 1687 | FQAPVTKAS |
| 1688 | GLGYVHRSN |
| 1689 | GWQSVWNAP |
| 1690 | GYNWVQEQR |
| 1691 | HAVMVYSDY |
| 1692 | HTKGVVHQP |
| 1693 | IARHVDHDL |
| 1694 | IQTEVHTVY |
| 1695 | KQQDVTMNA |
| 1696 | KYIDVWDAR |
| 1697 | LGVDVLADY |
| 1698 | MQVLVSMTP |
| 1699 | NTIDVQLGG |
| 1700 | QKAYVSVSG |
| 1701 | QSPAVNCAW |
| 1702 | RIEQVDVFN |
| 1703 | RIGGVADAD |
| 1704 | RWECVTGGY |
| 1705 | TCIRVWQWA |
| 1706 | TMRYVNTCV |
| 1707 | TSHNVANFG |
| 1708 | VEQMVMPPH |
| 1709 | VYTVVDEHS |
| 1710 | WFPVVWSHN |
| 1711 | YHVLVEGDR |
| 1712 | HGIALNLGT |
| 1713 | HRDYANSWN |
| 1714 | IGTRFNAQT |
| 1715 | ILDDCNCKC |
| 1716 | IPRMLNHVE |
| 1717 | ISYQPNPME |
| 1718 | KASELNQVL |
| 1719 | KHLDCNERE |
| 1720 | KLSNMNHPI |
| 1721 | KMEQGNMDS |
| 1722 | KMFHSNQEA |
| 1723 | KNWVCNLLT |
| 1724 | KTEVTNGPT |
| 1725 | LCIRKNYTH |
| 1726 | LCQFNNATD |
| 1727 | LEAECNSMQ |
| 1728 | LGDERNCGQ |
| 1729 | LHDNWNRYT |
| 1730 | LPFRSNNAQ |
| 1731 | LTVQNNGIR |
| 1732 | MCAYMNKKE |
| 1733 | MPNLSNSFQ |
| 1734 | NGAEMNWHE |
| 1735 | NGNQFNNCT |
| 1736 | NSTQPNLWF |
| 1737 | NYTIYNPAE |
| 1738 | PIETTNHMD |
| 1739 | PMYANNYFC |
| 1740 | PTQPLNQHG |
| 1741 | QAWCANTFH |
| 1742 | QCFYANRGD |
| 1743 | QHQQFNLTT |
| 1744 | QIDMMNCNR |
| 1745 | QQLPENLYQ |
| 1746 | QTDVINKFP |
| 1747 | QTGMENHQN |
| 1748 | QYSWSNINT |
| 1749 | RIEANNWGV |
| 1750 | RIICQNAVA |
| 1751 | SEKVINASC |
| 1752 | SIAVKNMCH |
| 1753 | SKRDNNCEV |
| 1754 | SMQRYNSKP |
| 1755 | SSLCRNMWP |
| 1756 | STYGRNLYP |
| 1757 | SWSNINHQT |
| 1758 | TCGEYNHRC |
| 1759 | TFQEKNVMH |
| 1760 | THQMTNTYS |
| 1761 | TIAQDNKEA |
| 1762 | TLITTNRQY |
| 1763 | TMVQNNMCF |
| 1764 | TRQPFNMSL |
| 1765 | TSSEFNRQR |
| 1766 | TSVACNSTD |
| 1767 | TTVFWNNFK |
| 1768 | TWVPDNADS |
| 1769 | TYQVNNWSD |
| 1770 | VANHHNEIQ |
| 1771 | VIGTANAAP |
| 1772 | VMGMINGAW |
| 1773 | VPLVINKEQ |
| 1774 | VSSQFNGPS |
| 1775 | VTHCKNVAC |
| 1776 | VWYPQNAVE |
| 1777 | WCNYLNQGM |
| 1778 | WQCTENWQN |
| 1779 | FYWAHNICE |
| 1780 | KYEILNCNH |
| 1781 | LYAMLNKFF |
| 1782 | MGNYQNTHY |
| 1783 | RKDEANGQT |
| 1784 | YFIIQNHLD |
| 1785 | YTRSNNWDY |
| 1786 | AAFMPNNYY |
| 1787 | AALFPNWSN |
| 1788 | AEECSNHNV |
| 1789 | AHSMTNLVG |
| 1790 | AWFDHNEHQ |
| 1791 | AYARSNRQD |
| 1792 | DATIANAQV |
| 1793 | DIAEKNRMM |
| 1794 | DWKTSNAEY |
| 1795 | EGDWYNYGT |
| 1796 | GIIFSNTCM |
| 1797 | GPPPYNAWF |
| 1798 | GSQRTNTSC |
| 1799 | GTSRYNAFI |
| 1800 | IALLTNDLA |
| 1801 | ICGRLNNNP |
| 1802 | INAPQNMSW |
| 1803 | ISGYHNVDE |
| 1804 | IYHTLNHCM |
| 1805 | KAEMSNAIT |
| 1806 | KAIPENSHR |
| 1807 | KIHSPNQDE |
| 1808 | LKSPHNENG |
| 1809 | LLEVKNHDH |
| 1810 | LSDLQNKGA |
| 1811 | LSHTSNMGH |
| 1812 | LTYLANQSD |
| 1813 | MGKSRNGSE |
| 1814 | MISMWNRSN |
| 1815 | MMDPTNFGS |
| 1816 | MNNITNWNP |
| 1817 | MTGRANKFD |
| 1818 | NNQWMNSLE |
| 1819 | NQIRNNMER |
| 1820 | NTYMHNREG |
| 1821 | NWNDSNDCC |
| 1822 | QCYTFNDTK |
| 1823 | QRDLANGQF |
| 1824 | QTASWNMEM |
| 1825 | RCEPFNWRE |
| 1826 | RMAQINQQG |
| 1827 | STALMNEYQ |
| 1828 | SWWLTNTAV |
| 1829 | TADFYNDQM |
| 1830 | TASSANLSW |
| 1831 | TFEICNSPQ |
| 1832 | TFLRLNDFV |
| 1833 | TFSQHNHKA |
| 1834 | TKCTTNQQC |
| 1835 | TSALNNNSK |
| 1836 | TSHDNNAQI |
| 1837 | TTFHINEYP |
| 1838 | VCVPKNGIH |
| 1839 | VGAQTNCLQ |
| 1840 | VYNLLNHYQ |
| 1841 | VYRIANTQY |
| 1842 | YAKSRNIVG |
| 1843 | HKESLGHHC |
| 1844 | HRHPCGHIP |
| 1845 | HSAPSFHTQ |
| 1846 | IDKLYWHWG |
| 1847 | IDQPIQHAS |
| 1848 | IGKMNDHGM |
| 1849 | IIQYNGHQA |
| 1850 | IKTELAHQY |
| 1851 | KAAGDSHTP |
| 1852 | KADAPIHWS |
| 1853 | KAECKPHHE |
| 1854 | KATELGHCG |
| 1855 | KCTAMRHKE |
| 1856 | KTEQATHTC |
| 1857 | KTSEWHHVG |
| 1858 | LATSRVHFG |
| 1859 | LGGWMEHSF |
| 1860 | LSDHLHHYT |
| 1861 | MCHHRHHAE |
| 1862 | MFRERIHHD |
| 1863 | NCIFIEHPT |
| 1864 | NKVGCQHSY |
| 1865 | NMKVDAHSN |
| 1866 | NSSYEEHDQ |
| 1867 | NTCFIGHNE |
| 1868 | PGSQYVHWI |
| 1869 | QASYRTHHD |
| 1870 | QCAIMRHYC |
| 1871 | QIAEIIHFG |
| 1872 | QIYPEHHKE |
| 1873 | QRKHYVHVF |
| 1874 | QWDTLRHTP |
| 1875 | RICFHQHWG |
| 1876 | RKECEDHGG |
| 1877 | RTLEWFHRG |
| 1878 | SFSWTYHQD |
| 1879 | TAVMYAHDC |
| 1880 | TREINDHGI |
| 1881 | TRGSMYHHC |
| 1882 | TTVRNFHAE |
| 1883 | TYCCYHHQG |
| 1884 | TYECAAHYP |
| 1885 | VAQPYIHSH |
| 1886 | VNKPAGHIQ |
| 1887 | VRDLLHHDM |
| 1888 | VYETLKHRG |
| 1889 | VYETMGHEE |
| 1890 | WYYYPAHHK |
| 1891 | YEVTIPHAI |
| 1892 | ETRWWPHIE |
| 1893 | MSCMKEHSP |
| 1894 | NCAPMEHHV |
| 1895 | QGLNYEHQE |
| 1896 | SCRHIYHEA |
| 1897 | SMNQAIHLE |
| 1898 | AAYMTDHMN |
| 1899 | ASTSIVHAH |
| 1900 | DSFLNRHTE |
| 1901 | DTCSRKHWM |
| 1902 | ESFYQRHVT |
| 1903 | ESVVGPHWD |
| 1904 | ETDVCRHGV |
| 1905 | HQNASMHMP |
| 1906 | HYIQIQHDY |
| 1907 | IMVALMHED |
| 1908 | IYNQFPHYG |
| 1909 | KHSPDHHFE |
| 1910 | KMAMLRHAT |
| 1911 | KSVGWCHAF |
| 1912 | LQQPIVHIE |
| 1913 | LTETEHHDQ |
| 1914 | MATHCLHWR |
| 1915 | MMPEIMHVL |
| 1916 | NMGLLYHTN |
| 1917 | NTEPMTHWN |
| 1918 | PMKPMHHGY |
| 1919 | QCTFYFHWR |
| 1920 | QHVQEIHGA |
| 1921 | QIYHNHHTA |
| 1922 | QSCVGLHQI |
| 1923 | SATSKSHTF |
| 1924 | SCDEYMHHP |
| 1925 | SCVLKVHFP |
| 1926 | SLCMHRHDF |
| 1927 | SMGPTVHQM |
| 1928 | TAGHEGHIC |
| 1929 | TCEWWIHMD |
| 1930 | TFGWFMHIP |
| 1931 | TFSQHSHKA |
| 1932 | TRQQLFHTQ |
| 1933 | TTGSTKHIG |
| 1934 | VMPCYWHTT |
| 1935 | WDGTKVHWL |
| 1936 | YHVILVHQA |
| 1937 | YPTVFEHSE |
| 1938 | HIGYMPRSY |
| 1939 | HLCPNQFSP |
| 1940 | HSNGLGPSC |
| 1941 | HSQIFMLSW |
| 1942 | IGSQIFTST |
| 1943 | IGSSIMSST |
| 1944 | IIHQQHNST |
| 1945 | ITSRRGMSE |
| 1946 | KAEPLRLSP |
| 1947 | KAFIRDESY |
| 1948 | KGDWTVKSY |
| 1949 | KGGHKDESP |
| 1950 | KIWAHMCSP |
| 1951 | KMFLLEQSD |
| 1952 | LASCNWRSQ |
| 1953 | LAYPCYASA |
| 1954 | LFSFLWRSA |
| 1955 | LIDQYTNST |
| 1956 | LIEQTCVSN |
| 1957 | LKVDFLLSN |
| 1958 | LMEQFDYSQ |
| 1959 | LQGEQSASD |
| 1960 | LYEQYKASY |
| 1961 | MQQYAYISH |
| 1962 | MSIRHMISY |
| 1963 | NCGVSMKSN |
| 1964 | NCHSKCYSS |
| 1965 | NTAHCWESD |
| 1966 | QGSFAHVSQ |
| 1967 | QHKVMGRSW |
| 1968 | QTRCDFLSH |
| 1969 | RADHYRASK |
| 1970 | RCEAEGGSK |
| 1971 | RFHVMETSM |
| 1972 | RSNIEHLSE |
| 1973 | RTDSFYESD |
| 1974 | RTIDPVQSS |
| 1975 | RTVEHQASA |
| 1976 | SLGCREISL |
| 1977 | SLHAWAISA |
| 1978 | SNFEFQKSH |
| 1979 | TCGLFSGSY |
| 1980 | TFTRPEQSQ |
| 1981 | TGKWISPSN |
| 1982 | TKENLAASY |
| 1983 | TMLVHHGSM |
| 1984 | TSFLLGVSS |
| 1985 | TTHDEGVSI |
| 1986 | VAAHPLTSD |
| 1987 | VFRMATESE |
| 1988 | VGQVIDRSW |
| 1989 | VTTRNPFST |
| 1990 | VYQVCTISD |
| 1991 | WCQRFQTSS |
| 1992 | WITEIELSG |
| 1993 | YFALSGFSS |
| 1994 | EIHHLPSSH |
| 1995 | GTAETYISA |
| 1996 | HSEQMHQSD |
| 1997 | LAKSCSSSQ |
| 1998 | NASLDMISV |
| 1999 | SSEPFTKSN |
| 2000 | VADMGPWSG |
| 2001 | APHSYAMSY |
| 2002 | ASHIKWNSH |
| 2003 | DAQQAPNSS |
| 2004 | EDWIMHASF |
| 2005 | EPSFESDSN |
| 2006 | ESKWWIQSD |
| 2007 | FGKDTFVSS |
| 2008 | GANNIYVSM |
| 2009 | GKSLMMTSF |
| 2010 | GNTQQPMSF |
| 2011 | GTAAMEQSC |
| 2012 | IDASLDMSW |
| 2013 | ISEPCYVSI |
| 2014 | LMNSYCVSH |
| 2015 | LNVEAELSF |
| 2016 | LSLPGVPSM |
| 2017 | NMTGIHVSP |
| 2018 | NTFATGNSP |
| 2019 | QCYESMTSW |
| 2020 | QICQTDASA |
| 2021 | SCVFRLASG |
| 2022 | SDPHAGPSD |
| 2023 | SLWILRMSN |
| 2024 | SSCGNMVSN |
| 2025 | THWQYEASH |
| 2026 | TNAQFSNSW |
| 2027 | TTGVSEGSA |
| 2028 | VANYHPYSM |
| 2029 | VIRYGDLSQ |
| 2030 | VMGPQPVSV |
| 2031 | VSVMLAASD |
| 2032 | HFYSQSTLP |
| 2033 | HGAIHAVLP |
| 2034 | HHECMWMFP |
| 2035 | HTEARMVEP |
| 2036 | IGNYYHKWP |
| 2037 | IIRHHEFAP |
| 2038 | IIWQQDIGP |
| 2039 | IQSECVVVP |
| 2040 | KNESAKQCP |
| 2041 | KSVNLCVDP |
| 2042 | KTSFSEMPP |
| 2043 | KTSMTFYNP |
| 2044 | LARSDCRQP |
| 2045 | LFYPEWDGP |
| 2046 | LGCDTEQMP |
| 2047 | LHEFHKSYP |
| 2048 | LQLRSDWCP |
| 2049 | LTIHSQREP |
| 2050 | MACLFRACP |
| 2051 | MEGAIGQNP |
| 2052 | MGANIAQMP |
| 2053 | MGSSRMIYP |
| 2054 | MHIFHSLCP |
| 2055 | MKSECMSGP |
| 2056 | MWGHISEMP |
| 2057 | NFHMKSMCP |
| 2058 | NLHDFIFAP |
| 2059 | NWNLWDTYP |
| 2060 | PEFEFFTTP |
| 2061 | QCDEQEYNP |
| 2062 | QGQRFYNYP |
| 2063 | QYARLDCLP |
| 2064 | RADIGSLCP |
| 2065 | RCILKREQP |
| 2066 | RETVAMDMP |
| 2067 | RFEFLMAPP |
| 2068 | SAYIGEQGP |
| 2069 | SLRVCIGQP |
| 2070 | STHLKLSDP |
| 2071 | STNLIGQNP |
| 2072 | TCGWSHAMP |
| 2073 | TGNLWQSMP |
| 2074 | TSQAPMCNP |
| 2075 | YCDRSGIWP |
| 2076 | YFYEAKLMP |
| 2077 | YNGYTSVCP |
| 2078 | YRDRFFAVP |
| 2079 | YSHPRDGAP |
| 2080 | ACQLLHWQP |
| 2081 | DWDSMCWWP |
| 2082 | EDTMAQMWP |
| 2083 | LCHFRLVGP |
| 2084 | NQTGHMPAP |
| 2085 | QAPAYMWGP |
| 2086 | WHSRKQWPP |
| 2087 | YTKYLMTEP |
| 2088 | AFATCSMMP |
| 2089 | APVCGQDPP |
| 2090 | DADERQILP |
| 2091 | DERNSFWFP |
| 2092 | DIHTNTIEP |
| 2093 | DSGWWATMP |
| 2094 | GLTMNRCYP |
| 2095 | GMFWLTQAP |
| 2096 | GWWVATMLP |
| 2097 | HAELTMNVP |
| 2098 | IACCSEAIP |
| 2099 | KGDYQADAP |
| 2100 | LEKSMMTAP |
| 2101 | LRYLSDADP |
| 2102 | MCRANGDMP |
| 2103 | MCTLRHWQP |
| 2104 | MFELFVMGP |
| 2105 | MSTPNMTQP |
| 2106 | NAEQWCCGP |
| 2107 | NMTPLMTLP |
| 2108 | QAQPNVNYP |
| 2109 | QAYTDDTGP |
| 2110 | QWGGNQQTP |
| 2111 | QYEEMAWGP |
| 2112 | SAYMLRYQP |
| 2113 | SGQQFIFCP |
| 2114 | SPGHNMVGP |
| 2115 | SSVHTLYFP |
| 2116 | SYNYEHAWP |
| 2117 | TCNMQRAVP |

### Example 8

AAV5 Variants with Tissue Tropism in Sciatic Nerve

This example describes engineered AAV5 variants with tissue tropism in sciatic nerve that were discovered using the methods and systems described in EXAMPLE 1-EXAMPLE 2.

CNS, liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive sciatic nerve tropism. The results are shown in FIG. 18N which shows the positional frequency of each amino acid in the 581 to 589 region of AAV5 VP1 in sciatic nerve tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in sciatic nerve over the frequency of that given amino acid residue occurring at the specified position in variants identified in all other harvested tissues was analyzed to identify a set of sequence rules for capsids that preferentially target sciatic nerve tissue. With reference to TABLE 15 below, and SEQ ID NO: 2 (AAV5 VP1), the following amino acids can, thus, be independently mutated, in any combination, at any one or more positions Xaa1-Xaa9, to provide a VP1 capsid with enhanced sciatic nerve tropism as compared wildtype AAV5 VP1 capsid (SEQ ID NO: 1), where sciatic nerve tropism here refers to properties that are preferred for sciatic nerve transduction over properties that are preferred for transduction of all other harvested tissues.

TABLE 15
Sciatic Nerve Tropism Rules

Xaa1 is selected from C, G, K, M, Q, R, or Y.
Xaa1 is selected from C, R, or Q.
Xaa1 is C.
Xaa2 is selected from A, C, F, I, Q, T, or V.
Xaa2 is selected from A, C, or I.
Xaa2 is A.
Xaa3 is selected from A, F, I, M, R, S, or T.
Xaa3 is selected from F, M, R, or S.
Xaa3 is R.
Xaa4 is selected from E, N, T, Q, or V.
Xaa4 is selected from E, T, or V.
Xaa4 is T.
Xaa5 is selected from F, H, Q, S, V, or Y.
Xaa5 is selected from F, V, or Y.
Xaa5 is V.
Xaa6 is selected from K, M, N, Q, S, or V.
Xaa6 is selected from M, N, or S.
Xaa6 is N.
Xaa7 is selected from K, M, Q, R, or T.
Xaa7 is selected from M, Q, or T.
Xaa7 is M.
Xaa8 is selected from A, G, H, Q, S, or V.
Xaa8 is selected from H or S.
Xaa8 is H.
Xaa9 is selected from C, E, I, K, or R.
Xaa9 is selected from C, I, or K.
Xaa9 is I.

TABLE 16 below provides sequences of the 581 to 589 region for variant AAV VP1 capsid polypeptides that were found in sciatic nerve tissue and comport to one or more of the rules provided in TABLE 15. The present disclosure, thus, provides for rAAVs composed of engineered AAV5 VP1 capsid polypeptides having a SEQ ID NO: 2, wherein the Xaa1 to Xaa9 region is any one of SEQ ID NO: 26118-SEQ ID NO: 26990, as disclosed in TABLE 16. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 16
Sequences of the 581 to 589 Region in AAV5 VP1
Capsid Polypeptide that Drive Sciatic
Nerve Tissue Tropism

| SEQ ID | 581-589 |
|---|---|
| NO | Sequence |
| 26118 | CQGYEHSQE |
| 26119 | CAHAMKWGS |
| 26120 | CGGQVEFGW |
| 26121 | CSSCYHRVF |
| 26122 | CCCEALMPQ |
| 26123 | CENPLCQSS |
| 26124 | CFGPEGYAG |
| 26125 | CHPQHHHSH |
| 26126 | CSSFYHRVF |
| 26127 | CKEPRKMRE |
| 26128 | CPVHQMDFT |
| 26129 | CIVKSNGSK |
| 26130 | CPGLQFDTP |
| 26131 | CHHFNMPSQ |
| 26132 | CCRQIWVQY |
| 26133 | CIIARSTAA |
| 26134 | CAEKQEYVQ |
| 26135 | CAHMQYAGP |
| 26136 | CANPLWAQT |
| 26137 | CAVKTHTCT |
| 26138 | CCNRNWNQQ |
| 26139 | CDYMHKLNE |
| 26140 | CEGRHTWGK |
| 26141 | CFHRHSTVP |
| 26142 | CFIAADNRN |
| 26143 | CFKHQMMMH |
| 26144 | CFSDTDNAM |
| 26145 | CFTNHWEMG |
| 26146 | CFVPMFNHN |
| 26147 | CFWLDWWCW |
| 26148 | CGYMHKLNE |
| 26149 | CHSIFNVKC |
| 26150 | CISAGMKND |
| 26151 | CKDLRCGCA |
| 26152 | CLGQMWAQW |
| 26153 | CLHQWLMPT |
| 26154 | CLIAPPHMD |
| 26155 | CLTSTSHCI |
| 26156 | CMCPGKEYP |
| 26157 | CMLAWQNDF |
| 26158 | CNPKCDAMM |
| 26159 | CNYDSHKMK |
| 26160 | CPLFLPALP |
| 26161 | CQNMKRIDW |
| 26162 | CQQPPRPEK |
| 26163 | CRIQSIHVH |
| 26164 | CSAGDEWGG |
| 26165 | CSKHATMAH |
| 26166 | CSKHVTMAH |
| 26167 | CSMLFHHTQ |
| 26168 | CTILNHQDA |
| 26169 | CTRPDEIKE |
| 26170 | CVAICQDGL |
| 26171 | CVHTISNCH |
| 26172 | CVTWTNRKL |
| 26173 | CVWSEDAGY |
| 26174 | CWCEKSHLG |
| 26175 | CWLCQCPRQ |
| 26176 | CYDHFQMKD |
| 26177 | CYQFHTGFW |
| 26178 | CHAAVANLY |
| 26179 | CTMCHDCSP |
| 26180 | CADPQERCD |
| 26181 | CMEVTCFVV |
| 26182 | CIGPFHCAN |
| 26183 | CWGPIMHPF |
| 26184 | CSESYRGPD |
| 26185 | CQQHFMFAP |
| 26186 | CGHLLRRAE |
| 26187 | CRRDGYAAM |
| 26188 | CPEHNQVGV |
| 26189 | CIHRFVSSP |
| 26190 | CDWMANVSV |
| 26191 | CCRMDNKKS |
| 26192 | CLNNSTCRG |
| 26193 | CDHVTKHPT |
| 26194 | CKYAFKMEK |
| 26195 | CDAEAEFRR |
| 26196 | CPFKLYSKG |
| 26197 | CSIQINNCR |
| 26198 | CMVSTQKLA |
| 26199 | CFCTYKIEH |
| 26200 | CVCWFWFFW |
| 26201 | CHMLNDCMS |
| 26202 | CMAHIWEGH |
| 26203 | CGCQVKCHT |
| 26204 | CSMPQQCME |
| 26205 | CWECCTDSG |
| 26206 | CIMLNGCFQ |
| 26207 | CIDVLFKHL |
| 26208 | CQMLCRPPV |
| 26209 | CAGIHLNEA |
| 26210 | CYMNFIVDG |
| 26211 | CYIRNMVNL |
| 26212 | CPKQANRSG |
| 26213 | CVLSRSTMR |
| 26214 | CEAGWCRSD |
| 26215 | CIQPTWDSY |
| 26216 | CYWPMTNCT |
| 26217 | CNGYWMCMS |
| 26218 | CSVWMQRDR |
| 26219 | CWILHHRCS |
| 26220 | CYNQFHTDN |
| 26221 | YAVMSPPAG |
| 26222 | YAVMSQPAG |
| 26223 | YAVMSRPAG |
| 26224 | YAVVSHPAG |
| 26225 | RAVEMQPCT |
| 26226 | IAEEGWACP |
| 26227 | QAPLATAIE |
| 26228 | FAPEFDNHC |
| 26229 | SAHMPKSVT |
| 26230 | EAYLYNIPM |
| 26231 | NAEQCMIGA |
| 26232 | RASMCDCFE |
| 26233 | SACPLWCGS |
| 26234 | DAMHKWIFA |
| 26235 | HAPCEQWAW |
| 26236 | MASIDKQHV |
| 26237 | AAQWPMEYT |
| 26238 | TAEWKLCGE |
| 26239 | QAEGGPNMP |
| 26240 | IATEHPDEW |
| 26241 | NALDEDCQH |
| 26242 | AAMLYNACM |
| 26243 | VADWIGHTP |
| 26244 | VAIQCNRSY |
| 26245 | GATTLTKCL |
| 26246 | YASTMHSIP |
| 26247 | QALVGNAAE |
| 26248 | SAICQNYDA |
| 26249 | DAHNDALAR |
| 26250 | AADECMLHT |
| 26251 | AAEKLFAWE |
| 26252 | AAFLGQLSV |
| 26253 | AANSRENDW |
| 26254 | AATNMSNVH |
| 26255 | DAEANGCDA |
| 26256 | DAGKLGQHD |
| 26257 | DAGWMNSWV |
| 26258 | DALPMHGQR |
| 26259 | DAMCSIRKD |
| 26260 | DARANCDAA |
| 26261 | EAEIAYGFM |
| 26262 | EAGCINVPS |
| 26263 | EAQLYSTKA |
| 26264 | EASTYFIWD |
| 26265 | FADTCPRHA |
| 26266 | FAELHGMQG |
| 26267 | FAHDSCESG |
| 26268 | FANWLKSFP |
| 26269 | FAQIIVHEH |
| 26270 | FATGCKQGT |
| 26271 | GANKTCCEG |
| 26272 | GASPKLGPV |
| 26273 | HADEPYEWR |
| 26274 | HAEYQQQMG |
| 26275 | HAGYIASPN |
| 26276 | HAIPFNDSQ |
| 26277 | IAFHRGADH |
| 26278 | IAGPQQQMT |
| 26279 | IAHHRGCHT |
| 26280 | IAHVMDCLE |
| 26281 | IALVMTSWE |
| 26282 | IAMMENRQQ |
| 26283 | KADMCPYII |
| 26284 | KADMKNPCD |
| 26285 | KAELGKLIE |
| 26286 | LAMPKPDKQ |
| 26287 | LANEQIDFT |
| 26288 | LATMHSGQC |
| 26289 | MAAWCHKSQ |
| 26290 | NADPCMWMQ |
| 26291 | NAFQKEACI |
| 26292 | NAHMYTKHS |
| 26293 | NAMWHTKVG |
| 26294 | NANQKWYWS |
| 26295 | NASFSNHQA |
| 26296 | PAYYGPGSE |
| 26297 | QAAPPCAMS |
| 26298 | QAIRGTHDQ |
| 26299 | QAMRFSENG |
| 26300 | QATAEYACD |
| 26301 | QAWMPVCHT |
| 26302 | RAIMSDWEC |
| 26303 | RALECNWFL |
| 26304 | RALTFDCHK |
| 26305 | SAEYSNDES |
| 26306 | SAHTFCAPR |
| 26307 | SAHVVLGEM |
| 26308 | SALENVPKY |
| 26309 | SAMTSSHQK |
| 26310 | SAMYTENIS |
| 26311 | SASTKVASE |
| 26312 | TADLNHGEQ |
| 26313 | TADVNLALD |
| 26314 | TADVTAQWL |
| 26315 | TAGHFKWEQ |
| 26316 | TAIRADWQS |
| 26317 | TANRMTNQP |
| 26318 | TASEAHMEK |
| 26319 | TATLWARGY |
| 26320 | TATQRGMFQ |
| 26321 | TAVHNTHVQ |
| 26322 | VAFPAQLGA |
| 26323 | VAISTSPTT |
| 26324 | VASFNQICA |
| 26325 | VATDRMWMC |
| 26326 | VAWMMLAKD |
| 26327 | YAEVFRFKG |
| 26328 | YAIDSSRME |
| 26329 | YANWLYDCT |
| 26330 | NAELQQADI |
| 26331 | TAAGYIVDA |
| 26332 | SAFQSSDVP |
| 26333 | EAYITYNKA |
| 26334 | TAEVRWPCP |
| 26335 | YAQRTCWPE |
| 26336 | YAMPLYFCE |
| 26337 | KAGYQEGSC |
| 26338 | AAMWWQWDR |
| 26339 | GAYSLHDWR |
| 26340 | HAETDHVGL |
| 26341 | FAQQDKQDR |
| 26342 | YAYHPQCKP |
| 26343 | QATGMHRWQ |
| 26344 | IADGFAAVA |
| 26345 | QAGNSMWAK |
| 26346 | MAMWIADTH |
| 26347 | MATCYMYAD |
| 26348 | IAKIGNCVW |
| 26349 | NANYWFYYD |
| 26350 | EASWYYPAI |
| 26351 | KACMSHQGR |
| 26352 | QAEENSHWC |
| 26353 | SAAWQFGGS |
| 26354 | RACGMRGDY |
| 26355 | DADAWWMMH |
| 26356 | NACDWKNDY |
| 26357 | SAIGKTKKM |
| 26358 | QAVRRWWFF |
| 26359 | AAIRYDPGF |
| 26360 | NAGWQGQVD |
| 26361 | NALPAQQHY |
| 26362 | IAYHYDQQN |
| 26363 | DATTSTECH |
| 26364 | HAVNFDCKK |
| 26365 | EASIQASWN |
| 26366 | QAIPRENPM |
| 26367 | DAHAFHCQP |
| 26368 | LATTTSYHC |
| 26369 | DASQIPITF |
| 26370 | VAADSRMMI |
| 26371 | PACKEWCFC |
| 26372 | IAHWKCHYP |
| 26373 | AAMHNLFET |
| 26374 | FAQQIINAS |
| 26375 | GAKYIQNTD |
| 26376 | EAGPRKRDV |
| 26377 | PAHINDQYM |
| 26378 | MAHAPPNSD |
| 26379 | MAMWAVWNK |
| 26380 | MAEQMERDF |
| 26381 | SATNCDLYP |
| 26382 | HAKQANGMV |
| 26383 | AADIFAIND |
| 26384 | TATCGIEVE |
| 26385 | AAIGNQTLA |
| 26386 | AAQYMGIMK |
| 26387 | KAPWAPMDS |
| 26388 | NAENESSIG |
| 26389 | QAKMILDGT |
| 26390 | DMRKTWEAC |
| 26391 | TGRIKDAGP |
| 26392 | PWRCMQGYY |
| 26393 | MVRQCDYKM |
| 26394 | AIRTTMCDW |
| 26395 | KSRRGKLTW |
| 26396 | TDRQIDQYH |
| 26397 | TSRSEAADL |
| 26398 | IMRHCYSNG |
| 26399 | KRRDGTRSY |
| 26400 | YIRVGGNNE |
| 26401 | QERSMNAWW |
| 26402 | EKRESFDCG |
| 26403 | DYREKYKCQ |
| 26404 | DHRFKYYDH |
| 26405 | DIRTTGCQT |
| 26406 | DSRTETFFG |
| 26407 | EDRITECGA |
| 26408 | ESRNWDDTD |
| 26409 | ESRTMNMYE |
| 26410 | EVRSTRKNH |
| 26411 | FPRQASSQQ |
| 26412 | FWRQSGKLQ |
| 26413 | HCRWVKYHK |
| 26414 | HTRWPHIWH |
| 26415 | IERVEYAVN |
| 26416 | INRLLMTCD |
| 26417 | INRVEFTTS |
| 26418 | ITRMNSETV |
| 26419 | KIRVYCTPE |
| 26420 | LPRWADVQQ |
| 26421 | MERWWLVEG |
| 26422 | NCRPMEDMR |
| 26423 | QERITGMVM |
| 26424 | QSRTQGNDH |
| 26425 | SDRTAMCAY |
| 26426 | TCRWFAHYS |
| 26427 | TGREVDHTS |
| 26428 | VTRCIPTQW |
| 26429 | WCRPQQPDF |
| 26430 | YVRVIDDKW |
| 26431 | GKRVEDTSC |
| 26432 | WCRCKRFIE |
| 26433 | EIRQFVKGA |
| 26434 | DVRSHDEMT |
| 26435 | WKRTAGVHF |
| 26436 | GHRPKLCDQ |
| 26437 | GPRHIEEDG |
| 26438 | ENRSPNNPA |
| 26439 | ASRSCMTQE |
| 26440 | ECRMAVQSC |
| 26441 | NNRRQFGYV |
| 26442 | TCRYEELAD |
| 26443 | APRDMDACV |
| 26444 | PPRTVARFK |
| 26445 | KERMQVSKA |
| 26446 | TNRCHDDRG |
| 26447 | EFRCRLIGS |
| 26448 | GTRIYGKVS |
| 26449 | KIRDCDGNV |
| 26450 | TLRAEMSFH |
| 26451 | VDRLFRMEY |
| 26452 | KQRDWENFA |
| 26453 | SCRMVYGRA |
| 26454 | FFRICGSYI |
| 26455 | EKRLFDRTY |
| 26456 | ESRQVQDCY |
| 26457 | PWRWHKSAC |
| 26458 | MKRAPTLDY |
| 26459 | AKRECTHIP |
| 26460 | SYRALFNGW |
| 26461 | DLRWLPYDL |
| 26462 | IIRQSEQYH |
| 26463 | IHRVVCRDH |
| 26464 | EVRIQCKID |
| 26465 | WERCLELNQ |
| 26466 | EQRHISRAP |
| 26467 | DSRTQGGLC |
| 26468 | TQREVETSY |
| 26469 | KQRIAYEWQ |
| 26470 | NTRIVCDLS |
| 26471 | KTYTHECVA |
| 26472 | AVATTVMMC |
| 26473 | EKMTHTCLQ |
| 26474 | MQMTQEWMA |
| 26475 | YTKTYVDCF |
| 26476 | KSGTCGYGQ |
| 26477 | SKNTVGIGH |
| 26478 | TLTTDKTYS |
| 26479 | ACGTGVMAL |
| 26480 | YYDTISPDG |
| 26481 | QMLTTAHNL |
| 26482 | IKETWRVYF |
| 26483 | QLDTYMEGR |
| 26484 | ACDTPFQYH |
| 26485 | AEGTGMGNF |
| 26486 | ALNTQEMCN |
| 26487 | ANFTFVRSE |
| 26488 | ATGTGWLDN |
| 26489 | DFSTHPGTE |
| 26490 | DHMTPWERQ |
| 26491 | DMITGMQGS |
| 26492 | DPSTCCCFM |
| 26493 | DSDTMMNHN |
| 26494 | DWSTQEVNY |
| 26495 | EFHTKNRDM |
| 26496 | EMVTEDFWQ |
| 26497 | EWMTQCGWH |
| 26498 | GSHTCHLEV |
| 26499 | GSLTQNDNL |
| 26500 | GSNTIMTCL |
| 26501 | GTGTHEDYD |
| 26502 | IEGTCDRAG |
| 26503 | IQTTEMTSQ |
| 26504 | IWCTSCEIA |
| 26505 | KICTFGRES |
| 26506 | KIITFISPH |
| 26507 | LCCTEECVN |
| 26508 | LESTSMWMY |
| 26509 | LGITMCTTM |
| 26510 | LQSTGIFYA |
| 26511 | LSQTHQKLH |
| 26512 | LTPTTNAWH |
| 26513 | MFCTSALRG |
| 26514 | MQETNQFMR |
| 26515 | MVATCFNND |
| 26516 | NMNTGQQPG |
| 26517 | NQGTCTQYS |
| 26518 | NQQTQVCMT |
| 26519 | PEATKPIKM |
| 26520 | RPCTYEYQD |
| 26521 | RWDTLYFDI |
| 26522 | SDETGVPAK |
| 26523 | SILTYQNVG |
| 26524 | SMATNHWAE |
| 26525 | SQFTQLTQA |
| 26526 | THYTDVLVV |
| 26527 | VCFTANLMG |
| 26528 | VGTTTMQSQ |
| 26529 | VHHTNTMAN |
| 26530 | VMSTDISQI |
| 26531 | VQTTYDRKG |
| 26532 | VRWTYMTNA |
| 26533 | VTPTLNRCQ |
| 26534 | YTHTPHLRE |
| 26535 | DMVTVNCID |
| 26536 | DSATFKSAD |
| 26537 | AVETGESAM |
| 26538 | QTNTMQMLS |
| 26539 | EIHTGRPGS |
| 26540 | EPETLWYVG |
| 26541 | ATDTYNLQE |
| 26542 | MEHTMDFGV |
| 26543 | EHITEQDLR |
| 26544 | ETSTHCCKV |
| 26545 | TYDTRNHCL |
| 26546 | PSNTGKFCQ |
| 26547 | KSQTVMYDF |
| 26548 | AECTEDNDD |
| 26549 | QNLTKEDQY |
| 26550 | WVPTPWRTH |
| 26551 | NWCTMYRYW |
| 26552 | AKPTHDVGS |
| 26553 | LFMTYSARN |
| 26554 | DRTTMVGWY |
| 26555 | GEMTRCFHN |
| 26556 | NDCTCRAIM |
| 26557 | IHFTSQSCL |
| 26558 | NSHTIMNGM |
| 26559 | YPDTADIFW |
| 26560 | SSMTAERFT |
| 26561 | SVNTMWYPD |
| 26562 | GRHTAFATG |
| 26563 | PWTTPENYT |
| 26564 | IWITCPYAA |
| 26565 | ACHTGQSGM |
| 26566 | QMVTQLNWE |
| 26567 | QWFTKGVGE |
| 26568 | QIKTHSNVF |
| 26569 | QTGTGHWFE |
| 26570 | NHWTTCDYS |
| 26571 | LMKTSPSYW |
| 26572 | NVWTNIAEM |
| 26573 | RQTTYDCLD |
| 26574 | GQNTTDRSN |
| 26575 | HVNTSCKQF |
| 26576 | NTHTGNDRL |
| 26577 | TYMTNLGDR |
| 26578 | KQKTQNRNA |
| 26579 | WKATPGGQC |
| 26580 | VTMTTKSVW |
| 26581 | TCLSVMNQY |
| 26582 | QSDAVHEPA |
| 26583 | YQERVRKDV |
| 26584 | DDLDVWQTS |
| 26585 | GCNHVHIGQ |
| 26586 | GIANVDDWE |
| 26587 | NQIDVQWLQ |
| 26588 | REVMVCILR |
| 26589 | LCHNVLDYY |
| 26590 | NQPCVNTYW |
| 26591 | TMAEVVINL |
| 26592 | QRMIVDALQ |
| 26593 | RMVRVSVHG |
| 26594 | FTVYVGARE |
| 26595 | ACAGVTWGI |
| 26596 | AFTNVTRWC |
| 26597 | ARNCVGSFC |
| 26598 | AVQSVLGCR |
| 26599 | DHCAVWNHE |
| 26600 | DISPVNRWH |
| 26601 | DSYRVLCLS |
| 26602 | DVIPVPKSC |
| 26603 | EQIHVTMAN |
| 26604 | EVYNVKGSA |
| 26605 | FLEFVHHGT |
| 26606 | FYSSVQRDQ |
| 26607 | GSHQVDILH |
| 26608 | MGDSVLTAH |
| 26609 | MMSNVPGEY |
| 26610 | NHVPVVGAM |
| 26611 | NTLLVDFDF |
| 26612 | PFTNVTRWC |
| 26613 | QNTCVIGAQ |
| 26614 | QQDAVCVSH |
| 26615 | QSFVVQGTY |
| 26616 | QWHAVTTNE |
| 26617 | RLDCVKESP |
| 26618 | RVVEVHAQV |
| 26619 | SDNFVITHS |
| 26620 | SQQFVHHIY |
| 26621 | SYYWVWMAG |
| 26622 | TNNVVIEWG |
| 26623 | TSMCVETQY |
| 26624 | TVIDVVRWH |
| 26625 | VFPDVSFWQ |
| 26626 | VIAQVFKYH |
| 26627 | VIGKVHHDN |
| 26628 | VTNRVAMPI |
| 26629 | VTSLVTNMG |
| 26630 | WYVPVNHLY |
| 26631 | WYVPVNQLY |
| 26632 | RWDFVWYIQ |
| 26633 | GVFYVGHWQ |
| 26634 | HGNMVLVIG |
| 26635 | NSVHVHASN |
| 26636 | NKEAVTGSS |
| 26637 | HPVKVDDFT |
| 26638 | SRLPVHECP |
| 26639 | WTLQVMKIN |
| 26640 | MRVQVFSDA |
| 26641 | SDSKVQWGM |
| 26642 | KSAQVYWHP |
| 26643 | QFMGVSSHS |
| 26644 | LFIKVKDTS |
| 26645 | HFHEVAWGS |
| 26646 | ALDRVSMAC |
| 26647 | WCEHVIMNP |
| 26648 | TKQPVHFLG |
| 26649 | YGNSVTCTS |
| 26650 | ESCLVRRGW |
| 26651 | EWEGVSQFR |
| 26652 | AEHMVVDHE |
| 26653 | TCNKVANMY |
| 26654 | GSYIVQNHC |
| 26655 | HHFHVNLDI |
| 26656 | KTSNVMDQM |
| 26657 | NVEDVGMIE |
| 26658 | VDMGVHVGS |
| 26659 | GGSYVSAPD |
| 26660 | VEDRVFACC |
| 26661 | WKAIVLEYW |
| 26662 | ASVMVAITG |
| 26663 | DSQSVGDSF |
| 26664 | INMPVSKDQ |
| 26665 | YRLHVWMKV |
| 26666 | YWEKVPCMK |
| 26667 | SGQPVDCMF |
| 26668 | FNWFVRAYI |
| 26669 | ACDQVNTVQ |
| 26670 | QTNQVVVVE |
| 26671 | FMMPVVDNY |
| 26672 | WCEAVGDYA |
| 26673 | SGFFVHTSN |
| 26674 | HFSHVDQCM |
| 26675 | NRCGVSKCC |
| 26676 | TSTFANEFT |
| 26677 | QRPIANDAM |
| 26678 | VQDKINYSY |
| 26679 | QTGHFNQRG |
| 26680 | EGTLWNVCV |
| 26681 | GVIPLNMPS |
| 26682 | MWTEFNEDG |
| 26683 | KMTNSNENS |
| 26684 | GRAHGNVCI |
| 26685 | QCEYSNCCY |
| 26686 | DVISRNGRC |
| 26687 | NMCEANHES |
| 26688 | EVAGENGNY |
| 26689 | YTGHTNSGR |
| 26690 | ACDFGNLGR |
| 26691 | ADVVTNINL |
| 26692 | AIEASNGGG |
| 26693 | ASESPNESG |
| 26694 | ATCQSNKCP |
| 26695 | ATTRHNTEA |
| 26696 | DGWSGNKMA |
| 26697 | DMKLQNDHP |
| 26698 | DMLKHNNAR |
| 26699 | DNNAMNDQQ |
| 26700 | DQQYSNVCC |
| 26701 | DRIWINWVG |
| 26702 | DVQKKNDKS |
| 26703 | EKDEINRAW |
| 26704 | ERFMANTGL |
| 26705 | ESSDTNYRQ |
| 26706 | EYHGLNLMA |
| 26707 | EYVWLNCGE |
| 26708 | FMHAGNLDA |
| 26709 | FVGCLNHYL |
| 26710 | GHIKLNVLP |
| 26711 | GIMQLNTEP |
| 26712 | GNCFQNKSN |
| 26713 | HPHVHNQKP |
| 26714 | HTVDGNVFP |
| 26715 | IDAVKNRND |
| 26716 | KEMAKNYEP |
| 26717 | KQDEWNCDR |
| 26718 | KQNCANLCE |
| 26719 | LLSDENTEC |
| 26720 | MKMEENSAS |
| 26721 | MLVQENNSY |
| 26722 | NLERCNTMM |
| 26723 | NQFFYNLQN |
| 26724 | NSCYMNSIE |
| 26725 | NYTQCNFSN |
| 26726 | QMAWENWFG |
| 26727 | QSCSDNYSG |
| 26728 | RCDQMNLHF |
| 26729 | RGDALNMVP |
| 26730 | SSFLLNHQY |
| 26731 | SWVFCNVGQ |
| 26732 | TTLNTNCSH |
| 26733 | YVYRMNGNC |
| 26734 | LMQWYNRCW |
| 26735 | VTEVFNQDV |
| 26736 | GNKLKNDQM |
| 26737 | YMGSTNIWM |
| 26738 | QLEECNPLS |
| 26739 | LVDCSNTCQ |
| 26740 | KLYGLNHSC |
| 26741 | SGALKNREE |
| 26742 | MIKLINCVN |
| 26743 | QPSRFNTAD |
| 26744 | MQEHKNFYG |
| 26745 | TTYPHNTHG |
| 26746 | IYKWDNKFH |
| 26747 | QRTQCNFSI |
| 26748 | KYWCKNEWD |
| 26749 | IEHCSNRYA |
| 26750 | MINWGNKCT |
| 26751 | VPLGFNFAG |
| 26752 | RENGYNATM |
| 26753 | QKSMLNSIQ |
| 26754 | EGQYWNYMT |
| 26755 | TFIYLNHFG |
| 26756 | DYFQYNEAP |
| 26757 | SCQLLNDGG |
| 26758 | AHEDPNTWC |
| 26759 | KNELENVIN |
| 26760 | VGGYSNQWV |
| 26761 | WDNWSNTQI |
| 26762 | HQYVHNWRQ |
| 26763 | VHHKTNRSY |
| 26764 | TMWEDNQNM |
| 26765 | DCDPYNMVD |
| 26766 | QISAWNEFF |
| 26767 | GNEDCNLAC |
| 26768 | MIMGYNFEF |
| 26769 | LPSHCNLGS |
| 26770 | TIDLSNKVT |
| 26771 | TTTITNRLC |
| 26772 | QCTAHNTCM |
| 26773 | DSMVYNRPQ |
| 26774 | KIQPTNHHE |
| 26775 | KGLIYNHHT |
| 26776 | NYGCHNCKY |
| 26777 | QIGRFAMSH |
| 26778 | RTGDAGMSP |
| 26779 | ILLPDDMLK |
| 26780 | HLDCLKMEM |
| 26781 | YSNSIQMSF |
| 26782 | YREHRTMSD |
| 26783 | GWYQKCMGD |
| 26784 | PTNYGWMWR |
| 26785 | GCYWNMMQP |
| 26786 | PNGHYWMIP |
| 26787 | ADYNNEMIQ |
| 26788 | AGSQMHMLS |
| 26789 | ASWSPQMNT |
| 26790 | ATGNKTMSY |
| 26791 | DLNQCQMYC |
| 26792 | EEVHSAMEG |
| 26793 | EGPALWMNT |
| 26794 | EGVHLPMGN |
| 26795 | EMEMEEMVG |
| 26796 | EQGYRVMSF |
| 26797 | EVFHDRMMG |
| 26798 | FCSEMVMNQ |
| 26799 | GNSKNDMHC |
| 26800 | GQCNTMMWQ |
| 26801 | HINAAVMHE |
| 26802 | IEPDCPMIP |
| 26803 | KIEAEEMHA |
| 26804 | KLVSKTMHH |
| 26805 | MMESALMDR |
| 26806 | NQDIMVMGQ |
| 26807 | NWACMGMGI |
| 26808 | PDWYNWMED |
| 26809 | PMKSPVMKD |
| 26810 | PQEEFMMTQ |
| 26811 | QELYLPMGC |
| 26812 | RFKHQMMMH |
| 26813 | RMDMKEMWG |
| 26814 | SQQLECMGP |
| 26815 | SRAEEYMPW |
| 26816 | TRVPSMMYK |
| 26817 | TYAANIMHV |
| 26818 | VCTHYVMQR |
| 26819 | VMKPCFMTP |
| 26820 | SKNLYTMAG |
| 26821 | LDGPIGMGT |
| 26822 | FCNYAVMSP |
| 26823 | TLPKMHMWP |
| 26824 | TKPLEDMPT |
| 26825 | ESAEPRMIP |
| 26826 | AEVPYVMQQ |
| 26827 | QHSRAYMWY |
| 26828 | TNFPTSMHG |
| 26829 | YISEYVMMV |
| 26830 | TTTNQFMFQ |
| 26831 | TVGIRHMKD |
| 26832 | QCFSTEMCQ |
| 26833 | ELEPDGMHV |
| 26834 | THARFKMKS |
| 26835 | YSVQAYMAN |
| 26836 | FQWRTEMNN |
| 26837 | PSIKHGMPK |
| 26838 | DVDFLKMHC |
| 26839 | IHSFMAMAQ |
| 26840 | ENCMSRMKP |
| 26841 | MLMHADMTQ |
| 26842 | FTEPMCMQG |
| 26843 | YHCCFAMQS |
| 26844 | PGGCYRMME |
| 26845 | SQKQFSMQN |
| 26846 | HMLHAIMES |
| 26847 | TNGVQIMHV |
| 26848 | ETEIRQMDG |
| 26849 | YSNSTIMNL |
| 26850 | YVKMCSMRN |
| 26851 | GTVECEMNA |
| 26852 | FWGMESMSC |
| 26853 | ASGHMTMCM |
| 26854 | NTSELLMAQ |
| 26855 | SEDCMDMYF |
| 26856 | STEFWVQHT |
| 26857 | WSSREYWHK |
| 26858 | TVMSAWSHM |
| 26859 | ETHYPYNHI |
| 26860 | FQIRFDRHH |
| 26861 | TSSHDEAHK |
| 26862 | TSYLRKIHS |
| 26863 | TRYRMRYHG |
| 26864 | GIWRTREHY |
| 26865 | RGSLMSFHE |
| 26866 | SYAAFLHHG |
| 26867 | LQFLTVVHL |
| 26868 | GVSRLPYHA |
| 26869 | AHFFEIPHS |
| 26870 | AILVEFPHG |
| 26871 | AWTPSHGHP |
| 26872 | DGCKMFNHC |
| 26873 | DGYIYHTHL |
| 26874 | DIGNQDRHS |
| 26875 | EDMPAVDHN |
| 26876 | EFDQYMTHC |
| 26877 | ERVPRYVHH |
| 26878 | ESVMQANHT |
| 26879 | EVCIYCSHV |
| 26880 | GEDEAWQHT |
| 26881 | GIVVADRHV |
| 26882 | GSLPWKNHV |
| 26883 | HIADKSTHR |
| 26884 | HILYAWWHS |
| 26885 | HSPFFMNHA |
| 26886 | HTYPLDHHF |
| 26887 | IYSDNQYHH |
| 26888 | KIVECVSHI |
| 26889 | KSMWSERHC |
| 26890 | KWWWAYTHK |
| 26891 | LIAMRIVHD |
| 26892 | LYCSPPNHC |
| 26893 | MEKRCGVHQ |
| 26894 | MIACDKVHY |
| 26895 | MMAKHTNHE |
| 26896 | NCVQWKYHQ |
| 26897 | NGCCLMTHL |
| 26898 | NYICTRKHP |
| 26899 | NYQNKLLHG |
| 26900 | QHMIREAHG |
| 26901 | QLSMLGVHF |
| 26902 | SCNMCVSHE |
| 26903 | SHKFMHYHW |
| 26904 | SHLNNITHQ |
| 26905 | SRNGSKRHA |
| 26906 | STKPWPEHN |
| 26907 | SWFECFHHE |
| 26908 | TIMMGVEHM |
| 26909 | VMFYTVHHE |
| 26910 | YSMEEHFHN |
| 26911 | YYVFMQLHA |
| 26912 | HWELIRCHD |
| 26913 | YEICKHGHF |
| 26914 | TCNVSWEHE |
| 26915 | HGNSFLVHW |
| 26916 | YITCQYSHH |
| 26917 | QVVIDPAHY |
| 26918 | RVWSLEWHL |
| 26919 | KYSPFQEHN |
| 26920 | YPQPCSCHS |
| 26921 | QVWSDGQHV |
| 26922 | SVTYHEWHP |
| 26923 | TCNVSWKHE |
| 26924 | MLFDFEEHN |
| 26925 | QTMCITIHP |
| 26926 | KTEKWASHE |
| 26927 | FCKCFMKHC |
| 26928 | QMSWGKYHQ |
| 26929 | KYINFDHHQ |
| 26930 | MKGYFVHHS |
| 26931 | NKPAMMQHE |
| 26932 | ITTDATGHF |
| 26933 | RDQKGLGHM |
| 26934 | MYLSFPVHE |
| 26935 | DVKYATLHD |
| 26936 | MYAGWLNHG |
| 26937 | AVIHEKPHG |
| 26938 | GTIRMPEHR |
| 26939 | QKHMILCHF |
| 26940 | QEAGYTRHN |
| 26941 | AKEHMPHHM |
| 26942 | TKQFFTAHC |
| 26943 | AFGLTTGHT |
| 26944 | YLESKTIHA |
| 26945 | TRDPFWHHD |
| 26946 | GVWMQGFHP |
| 26947 | AVQMQQQHI |
| 26948 | VKAWWHDHQ |
| 26949 | GIHQLIHHC |
| 26950 | TRQWMCVHC |
| 26951 | ILANPIRQI |
| 26952 | KHNPSRHDI |
| 26953 | NFYLSGFLI |
| 26954 | KQCSLAPTI |
| 26955 | GFHNTKLPI |
| 26956 | TILQQMEEI |
| 26957 | ACNPFWHSI |
| 26958 | AFTETKCSI |
| 26959 | AGSHFGRRI |
| 26960 | EVTRTQVQI |
| 26961 | FYLCNYGAI |
| 26962 | LEAKWQCSI |
| 26963 | LWMWQLSCI |
| 26964 | MKDRMHQVI |
| 26965 | NVAQPDFQI |
| 26966 | QITSGCHCI |
| 26967 | VTCPIQRTI |
| 26968 | VTHKLVDAI |
| 26969 | YPHKKCRCI |
| 26970 | YSLEKRFQI |
| 26971 | EPLYSWVGI |
| 26972 | ASIATMDRI |
| 26973 | TCGHTRFFI |
| 26974 | EHKGCMQAI |
| 26975 | VNDDYECGI |
| 26976 | NMPSLRTRI |
| 26977 | FKIEWSQDI |
| 26978 | EFVLGCQGI |
| 26979 | WDAYYTNGI |
| 26980 | QSMVACYTI |
| 26981 | ATNKYANAI |
| 26982 | WHFLMIFYI |
| 26983 | QQEAYILRI |
| 26984 | GGALARSSI |
| 26985 | EFDWLLGTI |
| 26986 | RRTDHGEPI |
| 26987 | WPNIKHQPI |
| 26988 | HQCQSRSYI |
| 26989 | EICMRSGFI |
| 26990 | IECSPRESI |

### Example 9

AAV5 Variants with Tissue Tropism in Skeletal Muscle

This example describes engineered AAV5 variants with tissue tropism in skeletal muscle that were discovered using the methods and systems described in EXAMPLE 1-EXAMPLE 2.

CNS, liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive skeletal muscle tropism. The results are shown in FIG. 18C which shows the positional frequency of each amino acid in the 581 to 589 region of AAV5 VP1 in skeletal muscle tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in skeletal muscle over the frequency of that given amino acid residue occurring at the specified position in variants identified in all other harvested tissues was analyzed to identify a set of sequence rules for capsids that preferentially target skeletal muscle tissue. With reference to TABLE 17 below, and SEQ ID NO: 2 (AAV5 VP1), the following amino acids can, thus, be independently mutated, in any combination, at any one or more positions Xaa1-Xaa9, to provide a VP1 capsid with enhanced skeletal muscle tropism as compared wildtype AAV5 VP1 capsid (SEQ ID NO: 1), where skeletal muscle tropism here refers to properties that are preferred for skeletal muscle transduction over properties that are preferred for transduction of all other harvested tissues.

TABLE 17
Skeletal Muscle Tropism Rules

Xaa1 is selected from A, E, H, M, P, Q, or S
Xaa1 is selected from P or Q
Xaa1 is Q
Xaa2 is selected from F, H, I, T, or V
Xaa2 is selected from T or V
Xaa2 is V
Xaa3 is selected from A, G, I, K, M, Q, R, S, T, or V
Xaa3 is selected from A, L, P, R, or T
Xaa3 is selected from L, P, or T
Xaa3 is P
Xaa4 is selected from D, E, G, P, or S
Xaa4 is selected from D, E, or S
Xaa4 is E
Xaa5 is selected from H, L, M, P, or V
Xaa5 is selected from L, M, or V
Xaa5 is L
Xaa6 is selected from E, H, N, or P
Xaa6 is P
Xaa7 is selected from A, H, N, Q or T
Xaa7 is H
Xaa8 is selected from I, K, M, P, or W
Xaa8 is selected from I, P, or W
Xaa8 is P
Xaa9 is selected from A, I, M, P, or V
Xaa9 is selected from A, M, or P
Xaa9 is M

TABLE 18 below provides sequences of the 581 to 589 region for variant AAV VP1 capsid polypeptides that were found in skeletal muscle tissue and comport to one or more of the rules provided in TABLE 17. The present disclosure, thus, provides for rAAVs composed of engineered AAV5 VP1 capsid polypeptides having a SEQ ID NO: 2, wherein the Xaa1 to Xaa9 region is any one of SEQ ID NO: 28991-SEQ ID NO: 29990, as disclosed in TABLE 18. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 18
Sequences of the 581 to 589 Region in
AAV5VP1 Capsid Polypeptide that Drive
Skeletal Muscle Tissue Tropism

| SEQ | |
|---|---|
| ID | 581-589 |
| NO | Sequence |
| 28991 | QAEIVCNSK |
| 28992 | QANGSMDHH |
| 28993 | QANWWVDAG |
| 28994 | QARTMDERP |
| 28995 | QAWEGCAGE |
| 28996 | QCAFIPNSA |
| 28997 | QCGNVMLMP |
| 28998 | QCHFLRTCK |
| 28999 | QCMCVEIAA |
| 29000 | QCNAQAELF |
| 29001 | QCQLTVSMS |
| 29002 | QCQWQKMEH |
| 29003 | QECYEKMEC |
| 29004 | QEHCMNYGP |
| 29005 | QEMFIQARG |
| 29006 | QENLTNGDN |
| 29007 | QFIMLGETN |
| 29008 | QHVQIMAGQ |
| 29009 | QIEGVNHAN |
| 29010 | QIERYWHAT |
| 29011 | QIHGVTIWQ |
| 29012 | QIHIWDGQS |
| 29013 | QIHLRECSF |
| 29014 | QIRAMLDSS |
| 29015 | QISIADCYN |
| 29016 | QIWNEVHAQ |
| 29017 | QKNEYWTAF |
| 29018 | QKNLVPSYW |
| 29019 | QKSFPGDGI |
| 29020 | QLMVYMYES |
| 29021 | QMEYTWMGG |
| 29022 | QMFQDHNQP |
| 29023 | QMHHWDVWG |
| 29024 | QMQPTCGQF |
| 29025 | QNENVVWMS |
| 29026 | QPMHQQVTF |
| 29027 | QPMSFNWKN |
| 29028 | QPNYVEHCG |
| 29029 | QPTMHMPRG |
| 29030 | QPVVTWQSH |
| 29031 | QQITTEHSG |
| 29032 | QQLCQLDRK |
| 29033 | QQYLCMSSD |
| 29034 | QSCATTSAQ |
| 29035 | QSFQAPRLA |
| 29036 | QSHRVMDIP |
| 29037 | QSHYCHTFS |
| 29038 | QSMSYQKEG |
| 29039 | QSNLKSMYC |
| 29040 | QSSSMACDN |
| 29041 | QSSWAWMAE |
| 29042 | QTGNWWQYG |
| 29043 | QTIFWLNVG |
| 29044 | QTISGFQDK |
| 29045 | QTLHLHTNM |
| 29046 | QTMRLEPYH |
| 29047 | QTNLLVDTS |
| 29048 | QTTAILKLD |
| 29049 | QTTLKMKED |
| 29050 | QVETPWSGA |
| 29051 | QVQNVWCPK |
| 29052 | QVVTMPDPL |
| 29053 | QVWDMNDNA |
| 29054 | QWFRQANYK |
| 29055 | QWLLLCHGN |
| 29056 | QYERLHTNM |
| 29057 | QYIPFERYW |
| 29058 | QYMTQAKKD |
| 29059 | QPSRFNTAD |
| 29060 | QKTANGWCH |
| 29061 | QMCVWMTRQ |
| 29062 | QAKMILDGT |
| 29063 | QTAEWYCGA |
| 29064 | QTNGHPYFP |
| 29065 | QFAQLWCAK |
| 29066 | QCTAHNTCM |
| 29067 | QAADSASAS |
| 29068 | QADRDGCYA |
| 29069 | QAGWINTLN |
| 29070 | QATFHATQK |
| 29071 | QCADKNVLE |
| 29072 | QCFNTCPCD |
| 29073 | QCKTVIMGA |
| 29074 | QDFQFDEVW |
| 29075 | QDTMMTYMS |
| 29076 | QDWFNLAHW |
| 29077 | QEEHQKFGM |
| 29078 | QEENENMGC |
| 29079 | QETQAAMRQ |
| 29080 | QEVTTTDPQ |
| 29081 | QFMVKNSNH |
| 29082 | QFNLTDYFH |
| 29083 | QFRGLEHMV |
| 29084 | QFVSTHHEA |
| 29085 | QGNTCHKDA |
| 29086 | QGRIYDQAT |
| 29087 | QGSWNFCPP |
| 29088 | QHAPLHNFI |
| 29089 | QHCPIVEQD |
| 29090 | QHREIVKLQ |
| 29091 | QHSTDSGDK |
| 29092 | QHVELQVMK |
| 29093 | QIACLQKQG |
| 29094 | QIHDQLKGN |
| 29095 | QIQEVQSSV |
| 29096 | QISIGCNSQ |
| 29097 | QISSSRDMY |
| 29098 | QITDWENFH |
| 29099 | QKDPLHVHS |
| 29100 | QKDQVWHAW |
| 29101 | QKEEQPKMG |
| 29102 | QKPELEQGP |
| 29103 | QLCNPLAPM |
| 29104 | QLPGIKMWD |
| 29105 | QMFGFQPGN |
| 29106 | QMHPMIRFF |
| 29107 | QMHSMTNCN |
| 29108 | QMRIWRMMW |
| 29109 | QMVPATPCN |
| 29110 | QNAFMDLLA |
| 29111 | QNCFRYECP |
| 29112 | QNETRQHCW |
| 29113 | QNMPVMQYR |
| 29114 | QNRPTECIG |
| 29115 | QNVKEYSAS |
| 29116 | QPADPNDGG |
| 29117 | QPAPGHRGQ |
| 29118 | QPILPNIWE |
| 29119 | QPQVTYDVR |
| 29120 | QQLFWDIGV |
| 29121 | QQPLMRTEE |
| 29122 | QQSTKMKWH |
| 29123 | QSDKKNRCA |
| 29124 | QSDQLSRIQ |
| 29125 | QSELGHELP |
| 29126 | QSLWQQLSM |
| 29127 | QSQPEWNEK |
| 29128 | QSSTDQEAW |
| 29129 | QSTENNWYS |
| 29130 | QSVQPCDDP |
| 29131 | QSVSQLTCC |
| 29132 | QTAATMTED |
| 29133 | QTAVKNIDN |
| 29134 | QTCNAEVFT |
| 29135 | QTCNCGKAF |
| 29136 | QTGQYATLK |
| 29137 | QTIPREAEC |
| 29138 | QTNLPVVKN |
| 29139 | QTQTWERQD |
| 29140 | QTWCKRIHF |
| 29141 | QTYMPHHAH |
| 29142 | QVCSMFGNA |
| 29143 | QVINVHGRW |
| 29144 | QVIRHECQY |
| 29145 | QVSNTATCL |
| 29146 | QVTQVQALG |
| 29147 | QVVEKHENR |
| 29148 | QVYHYCRRH |
| 29149 | QWHVGGTSA |
| 29150 | QWKCDKQSN |
| 29151 | QWRNPIYGP |
| 29152 | QWTQGYASP |
| 29153 | QYEWLNTCD |
| 29154 | QYFRFGFDP |
| 29155 | QYGNSLNAG |
| 29156 | QYVPAGYFA |
| 29157 | QADRIGQCL |
| 29158 | QANAMAEFP |
| 29159 | QCACTITMK |
| 29160 | QCASTMSMT |
| 29161 | QCELLNVLC |
| 29162 | QCIEYEATE |
| 29163 | QDGMEAKNR |
| 29164 | QDIKKWCSE |
| 29165 | QGFMEQQHT |
| 29166 | QHQAEMEGF |
| 29167 | QHTEMDCSN |
| 29168 | QIGGFQYYY |
| 29169 | QIKLQQWYD |
| 29170 | QKDEYLNWH |
| 29171 | QKDMHAYPL |
| 29172 | QKPRWFCCP |
| 29173 | QMSSGEWVR |
| 29174 | QNELRESYE |
| 29175 | QSKPMEHVQ |
| 29176 | QTDWSMFLP |
| 29177 | QTYFPWHWF |
| 29178 | QVCSRWTKS |
| 29179 | QWVEWPFPC |
| 29180 | AVEKVMEVQ |
| 29181 | AVKLNQTYK |
| 29182 | AVNGNVYGR |
| 29183 | CVEGMVFVP |
| 29184 | CVGQYNNDM |
| 29185 | CVLRQYDGG |
| 29186 | CVNVRGRSS |
| 29187 | CVRQKAETE |
| 29188 | CVTAASPWN |
| 29189 | DVAERHMHE |
| 29190 | DVCMATIGQ |
| 29191 | DVDPLPCEP |
| 29192 | DVGYQMKKQ |
| 29193 | DVICYSDHR |
| 29194 | DVISMSLMG |
| 29195 | DVLIDHDFF |
| 29196 | DVMHFLDLA |
| 29197 | DVQNMDIGV |
| 29198 | DVRDDEFGN |
| 29199 | EVCGMECSG |
| 29200 | EVDDKPSGH |
| 29201 | EVEYEAKTV |
| 29202 | EVFVADNNI |
| 29203 | EVGAWGASY |
| 29204 | EVKAANWQV |
| 29205 | EVQHHYVSG |
| 29206 | EVRMCNATH |
| 29207 | EVVKKPWDG |
| 29208 | FVFTHIDDY |
| 29209 | GVEPISRAA |
| 29210 | GVGQKCGHQ |
| 29211 | GVLAQQAQW |
| 29212 | GVLLPHHWM |
| 29213 | HVNQWGRYG |
| 29214 | HVQSIIHAE |
| 29215 | IVDWSNVLH |
| 29216 | IVGMANQEH |
| 29217 | IVHSLDAPG |
| 29218 | IVSFGYAQG |
| 29219 | IVSYLKTND |
| 29220 | IVYDMMADV |
| 29221 | KVDTQWKSV |
| 29222 | KVRPPCIYI |
| 29223 | LVDPHRPMM |
| 29224 | LVNHGDSYC |
| 29225 | LVTGNSWEQ |
| 29226 | MVIGTSLLA |
| 29227 | NVMGHDWVH |
| 29228 | NVQWFEQNI |
| 29229 | NVVWGMLYG |
| 29230 | NVYINLASS |
| 29231 | PVPHNYQWW |
| 29232 | PVRHCSVWF |
| 29233 | RVCTFCCCF |
| 29234 | RVDFNVYCK |
| 29235 | RVFMLEMYA |
| 29236 | RVNMNDIGE |
| 29237 | SVAMRQLDN |
| 29238 | SVFWPEHNP |
| 29239 | SVHICEDGW |
| 29240 | SVLSFVAQK |
| 29241 | SVNPYPHGI |
| 29242 | SVRTHMHLP |
| 29243 | SVTFDVGGP |
| 29244 | SVYICNMDQ |
| 29245 | TVAVNEHLA |
| 29246 | TVFVYDNPG |
| 29247 | TVLDAWTAE |
| 29248 | TVNSNRTGD |
| 29249 | TVNVFPIHE |
| 29250 | TVYDVNAFS |
| 29251 | VVMDAHCNL |
| 29252 | VVTDYMVGA |
| 29253 | WVKFANDCQ |
| 29254 | WVRMRNLEK |
| 29255 | YVAGLENNR |
| 29256 | YVKFSVNIN |
| 29257 | YVQLRIADD |
| 29258 | YVQTTAHSS |
| 29259 | MVTERKPWS |
| 29260 | SVLHVQALA |
| 29261 | WVHPYSYCA |
| 29262 | DVKSWAVCD |
| 29263 | LVVTKFTYV |
| 29264 | RVNGSTCRH |
| 29265 | AVESSKGYV |
| 29266 | AVGCFGKEP |
| 29267 | AVMEYILYK |
| 29268 | AVNDQWQWG |
| 29269 | AVQLGNSMF |
| 29270 | AVSAPLSKE |
| 29271 | AVVQGMLYP |
| 29272 | CVAHFDQCF |
| 29273 | CVDYHRREQ |
| 29274 | CVESGTGHQ |
| 29275 | CVIHRMDMP |
| 29276 | CVMNVIWDA |
| 29277 | CVNETDTAA |
| 29278 | CVSYNPSIR |
| 29279 | CVTINTISG |
| 29280 | DVNCCITCG |
| 29281 | DVNTYANRD |
| 29282 | DVRAMTNDW |
| 29283 | DVVEKCSWF |
| 29284 | DVVHMWWWS |
| 29285 | EVACYDMTG |
| 29286 | EVAMMINNN |
| 29287 | EVGTECQWL |
| 29288 | EVGTNHKQH |
| 29289 | EVKMFYKQY |
| 29290 | EVLDHISAG |
| 29291 | EVREEGVLT |
| 29292 | EVTHQLKNA |
| 29293 | EVTVFIAPW |
| 29294 | EVWNEESMF |
| 29295 | EVYIKQQYY |
| 29296 | FVPPSCYNY |
| 29297 | GVAAQPCSP |
| 29298 | GVAPKPDFE |
| 29299 | GVASCNRFD |
| 29300 | GVMPKYFFG |
| 29301 | GVNAKQQTR |
| 29302 | GVYFMCDFM |
| 29303 | HVQTAEWIP |
| 29304 | IVDWPANRY |
| 29305 | IVIGRWYSC |
| 29306 | IVMRALANN |
| 29307 | IVMSFHRNH |
| 29308 | IVTDCRDGL |
| 29309 | IVTTKQLCR |
| 29310 | KVDDHICPG |
| 29311 | KVGWIEWYA |
| 29312 | KVYPLDFKM |
| 29313 | LVDGCVWMH |
| 29314 | LVDYTGNED |
| 29315 | LVFYTSVRG |
| 29316 | LVTKEDCAY |
| 29317 | MVAVDGTEP |
| 29318 | MVDMKQLMF |
| 29319 | MVEANEHAM |
| 29320 | MVEIHPDYD |
| 29321 | MVHLRGDGE |
| 29322 | NVDSSPFLT |
| 29323 | NVHGIMWDN |
| 29324 | NVQTKMHLP |
| 29325 | NVTPHEFQA |
| 29326 | NVTTIEPAH |
| 29327 | NVVQVTQNA |
| 29328 | NVVSKDHGG |
| 29329 | NVWHGYWLN |
| 29330 | PVAHVPFGM |
| 29331 | PVTAPGFAH |
| 29332 | RVYNPMYWH |
| 29333 | SVAQEMEST |
| 29334 | SVCEDAFFD |
| 29335 | SVDAQAFVY |
| 29336 | SVDQESAYR |
| 29337 | SVERHGSDK |
| 29338 | SVHQWCDVT |
| 29339 | SVIAFHIHV |
| 29340 | SVLHTMMVK |
| 29341 | SVNEVIDQV |
| 29342 | SVNQESAYR |
| 29343 | SVNSKNHTQ |
| 29344 | SVSYGNHSV |
| 29345 | SVTDRDHQY |
| 29346 | TVAPQSSLW |
| 29347 | TVCMDANFK |
| 29348 | TVKDLRVVT |
| 29349 | TVLWKHDKA |
| 29350 | TVSQCSFFP |
| 29351 | TVTLSEKWH |
| 29352 | TVYGFPNEV |
| 29353 | VVGVAPVMP |
| 29354 | WVKAVTLEQ |
| 29355 | WVVQTFTFD |
| 29356 | YVDCRNDAG |
| 29357 | YVELYIVDQ |
| 29358 | YVRDTALCF |
| 29359 | YVTSALDTF |
| 29360 | CVSFDACFA |
| 29361 | DVDRLCSIP |
| 29362 | DVSQTNFSE |
| 29363 | DVVWSTKDQ |
| 29364 | DVWSSISDS |
| 29365 | EVDGGGKMQ |
| 29366 | EVFLAGLMS |
| 29367 | EVLLAGIGP |
| 29368 | EVTPMYCSD |
| 29369 | FVRGNLQTP |
| 29370 | GVCHNTGCT |
| 29371 | HVFILDRTC |
| 29372 | HVIPDPRPA |
| 29373 | IVETDGSLM |
| 29374 | IVKFECCNH |
| 29375 | LVYHSFVHE |
| 29376 | MVCREQENG |
| 29377 | MVEVATMPH |
| 29378 | RVDDYERTF |
| 29379 | SVIHYSWAL |
| 29380 | SVSAWNPMS |
| 29381 | DKPFEWDLL |
| 29382 | EQPHDPSST |
| 29383 | FDPNRSQDV |
| 29384 | GEPKCVMME |
| 29385 | GGPDIKHYI |
| 29386 | GGPSEQPRP |
| 29387 | HHPKFAEKG |
| 29388 | HHPQVLRGM |
| 29389 | HNPVWVQQE |
| 29390 | ICPLEQYEM |
| 29391 | IGPMGQYGR |
| 29392 | IMPEAKNTP |
| 29393 | KCPDEMNNI |
| 29394 | KTPFTRWLR |
| 29395 | KTPLMVASI |
| 29396 | MLPSFGDEM |
| 29397 | NGPQGLMAC |
| 29398 | NNPTNTEAR |
| 29399 | PCPDHHRWL |
| 29400 | PLPCPRSGY |
| 29401 | RIPPMMIML |
| 29402 | SSPPWGHPF |
| 29403 | SSPSHAEYL |
| 29404 | WMPLYCMHV |
| 29405 | WQPSHAYHF |
| 29406 | FCPCGQEMC |
| 29407 | SWPLPERPS |
| 29408 | LSPWPQTDQ |
| 29409 | EQPNMGDYG |
| 29410 | MLPLEMKNL |
| 29411 | FPPREGPEW |
| 29412 | AHPHCCRHP |
| 29413 | CAPQFNWQY |
| 29414 | CQPRYHWCY |
| 29415 | ESPNWGYMD |
| 29416 | GQPQQIWQA |
| 29417 | HHPHQANIY |
| 29418 | KDPLGESKW |
| 29419 | KMPLKQADF |
| 29420 | MFPQAPADG |
| 29421 | MFPVCQDRD |
| 29422 | MGPMKPMMD |
| 29423 | NPPLCQEYG |
| 29424 | PGPEQITAG |
| 29425 | PPPWFQSRS |
| 29426 | PQPCSWPAQ |
| 29427 | SGPWCGSAW |
| 29428 | SSPNVHDLD |
| 29429 | SWPFSWLIV |
| 29430 | TKPDSANWD |
| 29431 | VNPQEEFFD |
| 29432 | VQPPYIAMV |
| 29433 | VSPRDLHHM |
| 29434 | YFPQESENS |
| 29435 | YQPNWRSQT |
| 29436 | AHPWCLTPN |
| 29437 | DNPWLLTFR |
| 29438 | KQPTSASPI |
| 29439 | MDPLGPAAD |
| 29440 | NEPQICRKC |
| 29441 | NQPGRFRTV |
| 29442 | VPPRMTIEL |
| 29443 | YSPTTNGGP |
| 29444 | AISEHFWNV |
| 29445 | ANVELMTIK |
| 29446 | ATHEQGGQL |
| 29447 | CFCECSFCE |
| 29448 | CFDEFMLWG |
| 29449 | CITELINYT |
| 29450 | CLAEQITCQ |
| 29451 | CRSEGWIHT |
| 29452 | DGMEWDLCG |
| 29453 | DHVEIQHMS |
| 29454 | DTTEQMQIY |
| 29455 | EACEGAMLE |
| 29456 | ECEEPSLMK |
| 29457 | EEVESGHFG |
| 29458 | EILEEQESC |
| 29459 | ELIEKVYRW |
| 29460 | FTREDKDRC |
| 29461 | GAVEKMVQE |
| 29462 | GMEEVEREG |
| 29463 | HGEEIHWKD |
| 29464 | IWTEAAENC |
| 29465 | KGEEMYECG |
| 29466 | LREEYKLAG |
| 29467 | LTCENQQIP |
| 29468 | LWTESVMGN |
| 29469 | MALERCDVD |
| 29470 | MEEEYMFTA |
| 29471 | MMSEQCHLT |
| 29472 | MNIERDRNH |
| 29473 | NTCEPVQMI |
| 29474 | PTLEFLNLM |
| 29475 | RPNEPLDYS |
| 29476 | SKREIENCH |
| 29477 | SLCEMMQPH |
| 29478 | SMTEIVVAW |
| 29479 | SSNEGGCWM |
| 29480 | STKEDFDMF |
| 29481 | STVEKMQTL |
| 29482 | TTDECHGHP |
| 29483 | TTVEMECGM |
| 29484 | VEEERPFWS |
| 29485 | VRGERLSDL |
| 29486 | VTIERNIWQ |
| 29487 | VTRELWLPQ |
| 29488 | WCTEFSTRQ |
| 29489 | WNVETFLGY |
| 29490 | YIAEMEFMN |
| 29491 | KQHELMVRV |
| 29492 | PKHEDMRWW |
| 29493 | NQREWHGLA |
| 29494 | SECEVLCSL |
| 29495 | PHAETHWYT |
| 29496 | AAMESCAEI |
| 29497 | AEGEIEMWT |
| 29498 | AFVEFSDVT |
| 29499 | AGCETWNAP |
| 29500 | AMHEATDAQ |
| 29501 | AQSEWGSAT |
| 29502 | ARDESAEEH |
| 29503 | ATHEMHLDA |
| 29504 | CKKESGSYE |
| 29505 | DCDECQDDQ |
| 29506 | DKDERAEGM |
| 29507 | DQWEYEVQF |
| 29508 | EELEQQMCT |
| 29509 | EMHEWSRAN |
| 29510 | ESEEMHAYE |
| 29511 | GKIEARETD |
| 29512 | HMFERPRTQ |
| 29513 | HPSEECSWR |
| 29514 | KLVEMQESK |
| 29515 | KWCEVLWQP |
| 29516 | LEGEAYAQE |
| 29517 | LFSEFFEYE |
| 29518 | LSNEMTIDA |
| 29519 | LTIENPHDQ |
| 29520 | LYKEIGENI |
| 29521 | NAEEWWSCD |
| 29522 | NCVEAGSFA |
| 29523 | NMMEYERFS |
| 29524 | NNMEDRWGT |
| 29525 | NSNEHGAMW |
| 29526 | PTCEVPENC |
| 29527 | SFIEFPAMT |
| 29528 | SIDEQTFPP |
| 29529 | SKEEVQYIF |
| 29530 | SWTEISSVK |
| 29531 | TAAEMIYEY |
| 29532 | TWDEQTAQV |
| 29533 | TWRECQSSH |
| 29534 | VGNEDKCDP |
| 29535 | VMIEKTMMH |
| 29536 | VPVEVHKQA |
| 29537 | WGAENHGPW |
| 29538 | WKDEYTVDD |
| 29539 | YAREFGNMY |
| 29540 | YTDERAMLN |
| 29541 | CDAENSDGG |
| 29542 | CLDEPRTDF |
| 29543 | GHEEYWEHV |
| 29544 | HHREQKDHS |
| 29545 | HSAEQVLME |
| 29546 | MIQEIKEDN |
| 29547 | MWCEVMHCS |
| 29548 | SCRECDECP |
| 29549 | TPDEQSHYD |
| 29550 | TTAEYGMSI |
| 29551 | AFHHLGEFQ |
| 29552 | ALMPLTRWN |
| 29553 | AMHSLHYMF |
| 29554 | ASEYLASPM |
| 29555 | ATTLLHYKN |
| 29556 | AWWWLNAQG |
| 29557 | CNIQLHTHY |
| 29558 | DRVRLMMVA |
| 29559 | DWGYLRIWC |
| 29560 | DYSKLDVHY |
| 29561 | ERSTLPMSR |
| 29562 | FADYLAAAS |
| 29563 | FPYHLWVGG |
| 29564 | GAEKLHLVH |
| 29565 | GGIDLTLMM |
| 29566 | HADCLELSC |
| 29567 | HGEDLNEFL |
| 29568 | HHDDLAPWQ |
| 29569 | IICTLSLDS |
| 29570 | ILNVLESWE |
| 29571 | KERRLWSGV |
| 29572 | LFDRLKLNE |
| 29573 | LRSMLASYD |
| 29574 | MHGHLTQSI |
| 29575 | MSISLWGNA |
| 29576 | PENPLVTMS |
| 29577 | RNTCLNTGD |
| 29578 | SLSRLYWSN |
| 29579 | TIRSLEMVW |
| 29580 | VGIPLQAHC |
| 29581 | VMDGLGLVL |
| 29582 | VTGFLMIPT |
| 29583 | VTRDLOKEQ |
| 29584 | VYDYLINGA |
| 29585 | WHGRLMITC |
| 29586 | WYQFLDNQL |
| 29587 | YACLLDTLG |
| 29588 | YSYSLMSGW |
| 29589 | DKINLAVSY |
| 29590 | HMEWLEEQA |
| 29591 | ITVALNQGW |
| 29592 | KCADLIIRS |
| 29593 | HATGLVNFM |
| 29594 | ATMNLSWGL |
| 29595 | TYESLQNSW |
| 29596 | NLSNLVSCR |
| 29597 | ALDALHYCS |
| 29598 | ANIGLETGV |
| 29599 | ARVDLMSHQ |
| 29600 | AWTGLHVCN |
| 29601 | CRDGLPHTV |
| 29602 | CTFWLYFFR |
| 29603 | DFNYLNWFC |
| 29604 | DILDLVNSF |
| 29605 | DIYQLHPSY |
| 29606 | DLFCLNYDF |
| 29607 | DQHKLTVEN |
| 29608 | DTDTLMNSG |
| 29609 | DTELLLVQE |
| 29610 | DYMLLQGVY |
| 29611 | ECHLLEEQR |
| 29612 | ECRSLCFQH |
| 29613 | EDRDLSWTF |
| 29614 | EGMILDHTP |
| 29615 | EKANLTYGV |
| 29616 | EKRSLPACM |
| 29617 | EPHSLDICL |
| 29618 | EYFVLHVYD |
| 29619 | EYNSLLCHF |
| 29620 | FRKLLTRRF |
| 29621 | FWCYLVLMV |
| 29622 | GEDTLAAWG |
| 29623 | GGEFLAVAY |
| 29624 | HHMQLLRSG |
| 29625 | HPGKLFETH |
| 29626 | IGTALERQG |
| 29627 | KIVVLAKGE |
| 29628 | KQTVLHVHH |
| 29629 | KSLPLHDHF |
| 29630 | LFTCLQEQE |
| 29631 | LSGWLSQFG |
| 29632 | LTTHLPEWN |
| 29633 | MKTVLYVGD |
| 29634 | MNLHLPQWG |
| 29635 | NFHSLESTP |
| 29636 | NTVTLVQHE |
| 29637 | NYGKLGKSY |
| 29638 | PMDSLTRVY |
| 29639 | PWQNLAVNH |
| 29640 | RWQDLLNTG |
| 29641 | SQMHLFTSW |
| 29642 | SSFILLDHQ |
| 29643 | TFNKLSCMW |
| 29644 | THAMLDRNF |
| 29645 | TQEALMDGQ |
| 29646 | TQTNLAECA |
| 29647 | TSQLLMHGN |
| 29648 | TWGLLWKQY |
| 29649 | VKLKLSEGQ |
| 29650 | YTYALAHAT |
| 29651 | YYNNLNIHT |
| 29652 | AHAMLDRNF |
| 29653 | DYKYLSCQY |
| 29654 | EEMHLDNAL |
| 29655 | ENHNLENHG |
| 29656 | EQTVLHVHH |
| 29657 | HNIDLQNWD |
| 29658 | IDTLLYHAI |
| 29659 | KCEWLQWVP |
| 29660 | KPHPLDPFR |
| 29661 | LCSILVHWS |
| 29662 | MHGPLYCAE |
| 29663 | MWGGLDMVY |
| 29664 | NRAQLCRCN |
| 29665 | TAMWLHNMV |
| 29666 | TAVALFEMD |
| 29667 | TNVGLIDGS |
| 29668 | VQEWLGLYC |
| 29669 | CDGHDPFWY |
| 29670 | CEEKTPVWC |
| 29671 | CIYLKPDYQ |
| 29672 | CLQHMPRLQ |
| 29673 | DARSSPDCL |
| 29674 | DPWHEPSMG |
| 29675 | DRHTKPNST |
| 29676 | ETMYWPAMD |
| 29677 | FFRDCPYEY |
| 29678 | GNNRNPTYW |
| 29679 | GTSDFPEAV |
| 29680 | HNLKIPMME |
| 29681 | IASHNPSKQ |
| 29682 | IDFRPPNNT |
| 29683 | IPNGPPNNQ |
| 29684 | LGFRKPFTR |
| 29685 | LQYTFPIGQ |
| 29686 | LTEATPKWA |
| 29687 | MFQYYPYCD |
| 29688 | MGEDRPSMT |
| 29689 | MHFKYPQGG |
| 29690 | MPDMDPAAL |
| 29691 | PQHISPDPT |
| 29692 | PYDMIPCNR |
| 29693 | SAAFQPRAS |
| 29694 | SRQPVPLSK |
| 29695 | STARQPALE |
| 29696 | SWYMPPMYC |
| 29697 | TIFQPPFHY |
| 29698 | VMDTTPLHF |
| 29699 | WAVLGPQKF |
| 29700 | WSGFHPPTM |
| 29701 | WTVLGPQKF |
| 29702 | YANFSPLWS |
| 29703 | SNTPMPYHG |
| 29704 | TFVRKPSLM |
| 29705 | KDGRNPHCL |
| 29706 | TCEDDPSLR |
| 29707 | CWMIVPAWH |
| 29708 | AAEPIPTWP |
| 29709 | CHCMWPSGV |
| 29710 | CSENEPFHR |
| 29711 | DMGHCPHDM |
| 29712 | DSDNIPYCP |
| 29713 | DSWRFPGHP |
| 29714 | DSYVTPYFT |
| 29715 | DTGSTPVGT |
| 29716 | EINPEPNWK |
| 29717 | FKLWNPDSI |
| 29718 | FWRSVPEYW |
| 29719 | GISSWPSAH |
| 29720 | GPNTHPHMA |
| 29721 | GSSYVPICF |
| 29722 | GTDACPLHL |
| 29723 | GTNGVPKVN |
| 29724 | HDYPTPKMY |
| 29725 | HMTFKPTAQ |
| 29726 | HQYNEPHVS |
| 29727 | ICTNYPKMD |
| 29728 | IHLMKPAHI |
| 29729 | IHNKFPLTM |
| 29730 | LKDFDPAIH |
| 29731 | LKVLWPTEM |
| 29732 | MHLRDPHQH |
| 29733 | MMHGYPFYP |
| 29734 | MNWLVPYSD |
| 29735 | NAMAIPKEC |
| 29736 | NDAGEPTRE |
| 29737 | RAMMRPHGE |
| 29738 | RMEYPPEQF |
| 29739 | SCTHDPTQL |
| 29740 | SSQPVPVPV |
| 29741 | STYHTPFNN |
| 29742 | TCVMEPCIC |
| 29743 | TIQRYPDYG |
| 29744 | TNQPFPSSE |
| 29745 | TQNLEPHWN |
| 29746 | TSDQYPSSY |
| 29747 | TTARRPTYD |
| 29748 | VAMAHPMTV |
| 29749 | VAVDSPHEM |
| 29750 | VLGRSPQVM |
| 29751 | WTYAPPLNY |
| 29752 | AHSHMPVCL |
| 29753 | CKDLSPYKF |
| 29754 | EEMQYPMFM |
| 29755 | ETCKMPLSD |
| 29756 | HHELDPDWE |
| 29757 | IPHNEPKAG |
| 29758 | KECFIPCAF |
| 29759 | MTRIRPDAN |
| 29760 | WKAPGPITR |
| 29761 | AEHSPHHDF |
| 29762 | ASALHCHNQ |
| 29763 | ATADNHHPR |
| 29764 | CSSLYSHMP |
| 29765 | DMCKYSHFV |
| 29766 | ETHTRAHDC |
| 29767 | ETIFVDHLN |
| 29768 | FNIHICHFI |
| 29769 | GEASMEHMQ |
| 29770 | GRIFSKHPD |
| 29771 | GSNQIQHGN |
| 29772 | GYQPDQHCP |
| 29773 | HRLIEQHDT |
| 29774 | IMMLMSHLE |
| 29775 | ITCKFTHNG |
| 29776 | ITQHMNHFQ |
| 29777 | ITSWMAHPQ |
| 29778 | KEAVMNHWT |
| 29779 | KLFPWEHFG |
| 29780 | KLMFWIHHT |
| 29781 | KRMQPEHCA |
| 29782 | KTDHERHAY |
| 29783 | KTWLPYHTW |
| 29784 | KWLWNWHDW |
| 29785 | LCLSACHFE |
| 29786 | LHDNYCHET |
| 29787 | LSQDVHHDT |
| 29788 | MAFFSMHQG |
| 29789 | MCTDYGHEG |
| 29790 | MLKCNFHHQ |
| 29791 | MPEPRIHTR |
| 29792 | MSCPHFHVG |
| 29793 | NFEVCQHCE |
| 29794 | NHQNWMHMD |
| 29795 | RFILELHDS |
| 29796 | RPVNGVHLH |
| 29797 | SAGQIMHNS |
| 29798 | SWTWDWHVA |
| 29799 | THQADEHDA |
| 29800 | TTIQSNHCK |
| 29801 | TTMQQGHDL |
| 29802 | WSANQMHSH |
| 29803 | YIERTNHTP |
| 29804 | FRDVCMHDV |
| 29805 | FANDFTHNM |
| 29806 | GQLSTQHHM |
| 29807 | ASKQGTHDY |
| 29808 | AAEPYKHSK |
| 29809 | ASNTYDHSL |
| 29810 | CAMPGSHQI |
| 29811 | CDVKDAHHD |
| 29812 | DGCDMTHTR |
| 29813 | DGWLYQHTY |
| 29814 | DTMSKNHCT |
| 29815 | DTYAAGHSA |
| 29816 | EDTLTLHEN |
| 29817 | EFTLFQHEN |
| 29818 | ENVDRQHHM |
| 29819 | EPNMCCHHR |
| 29820 | EYTDVMHDA |
| 29821 | FAAVCHHPI |
| 29822 | FDTFSVHRL |
| 29823 | FTDCMGHTM |
| 29824 | GAYQPCHYP |
| 29825 | GSWHCVHCP |
| 29826 | HAAPQCHYS |
| 29827 | KMEFQLHPG |
| 29828 | LDGAMSHMC |
| 29829 | MHVTPRHPP |
| 29830 | MQLIDHHGP |
| 29831 | MSKVDRHCA |
| 29832 | MTMLGGHCI |
| 29833 | NCMYEMHWQ |
| 29834 | NFYDSLHCF |
| 29835 | NGNLTAHPN |
| 29836 | NIELRDHGQ |
| 29837 | PCHRKKHPK |
| 29838 | RCGRTAHNH |
| 29839 | REWYHCHYM |
| 29840 | SKYPINHVQ |
| 29841 | SSEFAVHCG |
| 29842 | TINVKHHFG |
| 29843 | WDWFSYHAE |
| 29844 | WEAKCAHSV |
| 29845 | WPVLNDHEH |
| 29846 | WPVLNDHGH |
| 29847 | YHEKSCHFG |
| 29848 | CQVSYQHYP |
| 29849 | DGTVCVHMG |
| 29850 | GAESCCHFG |
| 29851 | GELMHRHMD |
| 29852 | HTQHHSHHL |
| 29853 | ISSNFDHFE |
| 29854 | KMQNCAHQH |
| 29855 | LSSLHDHPE |
| 29856 | NTQWEAHSP |
| 29857 | SFSSNAHLQ |
| 29858 | STECYYHGI |
| 29859 | TSTHIEHDT |
| 29860 | YRVNCGHPN |
| 29861 | AKDLISKPH |
| 29862 | AMCNDIAPW |
| 29863 | DILPKVYPE |
| 29864 | DLEWQHCPP |
| 29865 | DMEPQYWPA |
| 29866 | DTLGPEAPH |
| 29867 | EAMMMYQPN |
| 29868 | EIMAAWTPP |
| 29869 | EQGSVWTPP |
| 29870 | EWFHQMSPH |
| 29871 | FMEDNTKPR |
| 29872 | HTIWSHDPW |
| 29873 | LATQICFPT |
| 29874 | LRMDCVPPY |
| 29875 | LTGPNLSPA |
| 29876 | MGNRSGSPS |
| 29877 | MPSHWYRPI |
| 29878 | MTHKDIGPS |
| 29879 | NFCKGMNPY |
| 29880 | PATKTHDPF |
| 29881 | SALNAAPPP |
| 29882 | SWCRYHEPL |
| 29883 | TFMHESEPH |
| 29884 | WQYTONCPA |
| 29885 | YLGWICAPC |
| 29886 | YCECGHWPN |
| 29887 | MPWLDKPPC |
| 29888 | CQMLCRPPV |
| 29889 | PIWQGYWPW |
| 29890 | YDYWMQAPT |
| 29891 | EWVVQQIPQ |
| 29892 | ADHWEKGPF |
| 29893 | AIDPINRPN |
| 29894 | AMEKPTLPI |
| 29895 | CTNFVNRPG |
| 29896 | DIVNHESPG |
| 29897 | GDESGLVPE |
| 29898 | GNESGLVPE |
| 29899 | GPKWGRSPL |
| 29900 | HDAPMFTPG |
| 29901 | ICMICATPI |
| 29902 | KWEHGFMPF |
| 29903 | LCKVGMEPE |
| 29904 | MEETVTRPG |
| 29905 | MEKRKGQPV |
| 29906 | NDLSPAEPT |
| 29907 | NMAMAKQPG |
| 29908 | RCSKVKWPA |
| 29909 | RFVVASYPY |
| 29910 | RHVSIMPPV |
| 29911 | SAVNKCVPG |
| 29912 | SETRNNFPY |
| 29913 | SIMRCKFPN |
| 29914 | STGPILDPP |
| 29915 | TDCCVGPPQ |
| 29916 | VGNVIARPV |
| 29917 | YCVCIQRPF |
| 29918 | GLIKIVKPW |
| 29919 | HEHTAFRPE |
| 29920 | IQIRANCPN |
| 29921 | MEGHTGNPC |
| 29922 | TPCIWHSPF |
| 29923 | AMEIQYMSM |
| 29924 | ASTNEEKSM |
| 29925 | CIEQKHLCM |
| 29926 | EAGIRAEDM |
| 29927 | EMSSSNMAM |
| 29928 | FFCKYNEVM |
| 29929 | FHHNSRLDM |
| 29930 | GGHSMTKAM |
| 29931 | GIVRQCCIM |
| 29932 | GIYYEQDGM |
| 29933 | GRRAELGLM |
| 29934 | GTMQVTGAM |
| 29935 | GYSSTHVQM |
| 29936 | IAVQTGSDM |
| 29937 | KCMPNVGWM |
| 29938 | KLKPICMKM |
| 29939 | LCWMAGAQM |
| 29940 | NAICTLEEM |
| 29941 | NCEFWKMFM |
| 29942 | NCVQTNSEM |
| 29943 | SADSEEGHM |
| 29944 | SKMSNQDAM |
| 29945 | SKSDVQSMM |
| 29946 | SPLNRNCLM |
| 29947 | VWLQSLDRM |
| 29948 | WMGHKHKQM |
| 29949 | MLTPVQATM |
| 29950 | KTSNVMDQM |
| 29951 | AKCDVTGGM |
| 29952 | AYMAYHTEM |
| 29953 | CTTASMSYM |
| 29954 | EIGSRNRRM |
| 29955 | ERSFHNSIM |
| 29956 | ESRIGQECM |
| 29957 | EYFNILRNM |
| 29958 | FATWHLYDM |
| 29959 | FDCWANNNM |
| 29960 | FIWDCGDIM |
| 29961 | FKTFGSREM |
| 29962 | GDDHRVLTM |
| 29963 | GGYHKFPVM |
| 29964 | GNDIFMVIM |
| 29965 | GNTPCRGLM |
| 29966 | LIACRENEM |
| 29967 | LKATYEYVM |
| 29968 | LNDIKWYVM |
| 29969 | LQIRAWEYM |
| 29970 | MLKVQCTHM |
| 29971 | PQDFKHNYM |
| 29972 | REFCGALMM |
| 29973 | RFGREAGGM |
| 29974 | RFTGHMGAM |
| 29975 | RHESINRYM |
| 29976 | RSDLKVCAM |
| 29977 | RYNLQIMWM |
| 29978 | SFMLKWWAM |
| 29979 | SMFGDRQAM |
| 29980 | SNCDNWSAM |
| 29981 | SPLHYLVQM |
| 29982 | SYRPFRKMM |
| 29983 | TLMQPGYQM |
| 29984 | VPDHIVGMM |
| 29985 | VSSSDGWNM |
| 29986 | WTAHMYTNM |
| 29987 | WTYHSMAQM |
| 29988 | WWWGNFMFM |
| 29989 | YCNQQQWTM |
| 29990 | YEQVITVMM |

### Example 10

AAV5 Variants with Tissue Tropism in Spinal Cord

This example describes engineered AAV5 variants with tissue tropism in spinal cord that were discovered using the methods and systems described in EXAMPLE 1-EXAMPLE 2.

CNS, liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive spinal cord tropism. The results are shown in FIG. 18O which shows the positional frequency of each amino acid in the 581 to 589 region of AAV5 VP1 in spinal cord tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in spinal cord over the frequency of that given amino acid residue occurring at the specified position in variants identified in all other harvested tissues was analyzed to identify a set of sequence rules for capsids that preferentially target spinal cord tissue. With reference to TABLE 19 below, and SEQ ID NO: 2 (AAV5 VP1), the following amino acids can, thus, be independently mutated, in any combination, at any one or more positions Xaa1-Xaa9, to provide a VP1 capsid with enhanced spinal cord tropism as compared wildtype AAV5 VP1 capsid (SEQ ID NO: 1), where spinal cord tropism here refers to properties that are preferred for spinal cord transduction over properties that are preferred for transduction of all other harvested tissues.

TABLE 19
Spinal Cord Tropism Rules

Xaa1 is selected from A, C, K, Q, R, S, or W
Xaa1 is selected from K, R, or W
Xaa1 is K
Xaa2 is selected from H, I, K, L, T, V, or W
Xaa2 is selected from H, I, or T
Xaa2 is I
Xaa3 is selected from C, F, G, H, I, K, N, or R
Xaa3 is selected from F, I, or R
Xaa3 is I
Xaa4 is selected from I, M, Q, S, or V
Xaa4 is selected from I, M, or V
Xaa4 is V
Xaa5 is selected from H, K, Q, T, W, or Y
Xaa5 is selected from T, W, or Y
Xaa5 is Y
Xaa6 is selected from H, L, N, Q, R, W, or Y
Xaa6 is selected from L, N, R, or Y
Xaa6 is Y
Xaa7 is selected from D, H, P, Q, or R
Xaa7 is R
Xaa8 is selected from D, F, L, S, T, or Y
Xaa8 is selected from S, T, or Y
Xaa8 is T
Xaa9 is selected from C, I, N, P, R, S, or Y
Xaa9 is selected from I, P, or R
Xaa9 is I

TABLE 20 below provides sequences of the 581 to 589 region for variant AAV VP1 capsid polypeptides that were found in spinal cord tissue and comport to one or more of the rules provided in TABLE 19. The present disclosure, thus, provides for rAAVs composed of engineered AAV5 VP1 capsid polypeptides having a SEQ ID NO: 2, wherein the Xaa1 to Xaa9 region is any one of SEQ ID NO: 34991-SEQ ID NO: 35437, as disclosed in TABLE 20. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 20
Sequences of the 581 to 589 Region
in AAV5 VP1 Capsid Polypeptide that
Drive Spinal Cord Tissue Tropism

| SEQ | |
|---|---|
| ID | 581-589 |
| NO | Sequence |
| 34991 | KYNMLYHMD |
| 34992 | KGESQCTSQ |
| 34993 | KGQHKENRS |
| 34994 | KWHGGPGVV |
| 34995 | KRMLYPMCF |
| 34996 | KATNADGET |
| 34997 | KQSDKWGDD |
| 34998 | KIMHSWYND |
| 34999 | KKEQDHWDK |
| 35000 | KASLGACVE |
| 35001 | KGGFYGESA |
| 35002 | KQNPSMRQD |
| 35003 | KSFSWDSSA |
| 35004 | KFLIAFEVT |
| 35005 | KEFMPCCHR |
| 35006 | KEGITGLEL |
| 35007 | KTCTEWPGE |
| 35008 | KVNRSNRIW |
| 35009 | KVTNYYHSG |
| 35010 | KYSPTPFMY |
| 35011 | KCEGEEVGR |
| 35012 | KQEYIMRQR |
| 35013 | KWDDMHTQM |
| 35014 | KNETFIAST |
| 35015 | KAVTRADAS |
| 35016 | KVAEGQNML |
| 35017 | KHLDMTSCH |
| 35018 | KATRDPKFP |
| 35019 | KTLPCSNSE |
| 35020 | KFNTKDQLM |
| 35021 | KGGLSTTNA |
| 35022 | KIQPTNHHE |
| 35023 | KTGWDSHWS |
| 35024 | KGCVPWSKI |
| 35025 | KMEVNMSEL |
| 35026 | KTLHMLESK |
| 35027 | KTGATNLAW |
| 35028 | KCRYKDCQC |
| 35029 | KSIMMSQLD |
| 35030 | KSGMIQEET |
| 35031 | KAIPQGSSC |
| 35032 | KGQRRDSVS |
| 35033 | KIEYGHWIH |
| 35034 | KAHDITWNL |
| 35035 | KSAQVYWHP |
| 35036 | KIQPMMVKA |
| 35037 | KEFQLQMEP |
| 35038 | KSTMLEHHD |
| 35039 | KRFVDMPEM |
| 35040 | KYSPFQEHN |
| 35041 | KNCIMLASN |
| 35042 | KSFQEQGGA |
| 35043 | KTMGIGGGP |
| 35044 | KVEGYGWTR |
| 35045 | KPLTYTVGS |
| 35046 | KQATNNNGW |
| 35047 | KVEIINWHP |
| 35048 | KSEHHWINA |
| 35049 | KGNPLDGDT |
| 35050 | KCDDRNTQH |
| 35051 | KLMYKVMSC |
| 35052 | KOKTQNRNA |
| 35053 | KCADLIIRS |
| 35054 | KHEPMCVQL |
| 35055 | KIGCMYSSQ |
| 35056 | LIAAQKNFK |
| 35057 | QIMQKYQYS |
| 35058 | WIPAMTYWC |
| 35059 | WIPAMTYWY |
| 35060 | WINMPHDRH |
| 35061 | DIVCLMNST |
| 35062 | MIRPLTHCK |
| 35063 | FIIRCMLIW |
| 35064 | TISSVFLLD |
| 35065 | EICQHMGMA |
| 35066 | SIVNHNEAR |
| 35067 | GIELPVKHH |
| 35068 | EICSRGRTI |
| 35069 | GIKYKDVLS |
| 35070 | GIKFFVDNC |
| 35071 | GIIERYGDC |
| 35072 | NICAKHRMS |
| 35073 | RIQKLKWCQ |
| 35074 | SIDVPPTAN |
| 35075 | TIVWSECAW |
| 35076 | NIDAVERIW |
| 35077 | TIGWKELNE |
| 35078 | TIMFGCLAE |
| 35079 | GIFMEVAWC |
| 35080 | RICPLFCIK |
| 35081 | LIQSYPHQC |
| 35082 | MISAPPDTC |
| 35083 | QIGPKAFGW |
| 35084 | DIACLDQWQ |
| 35085 | GILVQGGSM |
| 35086 | TIRPHHAKS |
| 35087 | AIFHACNSG |
| 35088 | RIVDGNACG |
| 35089 | CIHSQGMVC |
| 35090 | QILQWDCPS |
| 35091 | QIYSLASTW |
| 35092 | SIKFHERQH |
| 35093 | TILQQMEEI |
| 35094 | AIGPDRSNN |
| 35095 | TIHPAWVTL |
| 35096 | QISMKGEHG |
| 35097 | EIIQAHLIN |
| 35098 | CIIGRQEWL |
| 35099 | TIWVHEYGM |
| 35100 | SIAQDQVWS |
| 35101 | PIWQGYWPW |
| 35102 | IIKFLWRPT |
| 35103 | DITSLGCIM |
| 35104 | WINEANCLM |
| 35105 | GIKKPNEDN |
| 35106 | EIRQFVKGA |
| 35107 | TILPKKTLC |
| 35108 | DIGRFTAYY |
| 35109 | TISQIPWVR |
| 35110 | TIVVIPIKS |
| 35111 | LINSIVCGD |
| 35112 | GIPLWVFID |
| 35113 | FIDEKQCHE |
| 35114 | DICQKQSPN |
| 35115 | NIGVIPHIQ |
| 35116 | CIDVLFKHL |
| 35117 | QIIYRCRST |
| 35118 | EIHMFGQSE |
| 35119 | QIHSEHCAT |
| 35120 | HIHMSDRGA |
| 35121 | AIGCFQNGG |
| 35122 | HIKVEHEEV |
| 35123 | LIEKQRWML |
| 35124 | GIWVRDTYN |
| 35125 | EICMRSGFI |
| 35126 | CIQPTWDSY |
| 35127 | QISKPSVQC |
| 35128 | SGITFPGSN |
| 35129 | DFIPDWSST |
| 35130 | EMISNNCHR |
| 35131 | EMINACFQA |
| 35132 | APIFVCDCC |
| 35133 | YSIIWRVYR |
| 35134 | RKIESYCIT |
| 35135 | EKIMVKRAF |
| 35136 | ATISKLECQ |
| 35137 | LKICFSPTV |
| 35138 | ELIYKIKTD |
| 35139 | ARIWNDSMA |
| 35140 | DQILPMNGV |
| 35141 | IHIHFNWFY |
| 35142 | RGIYCYYVC |
| 35143 | WRIYEEDPT |
| 35144 | YKIGAAECD |
| 35145 | DPILQTYLS |
| 35146 | DQILSMNGV |
| 35147 | GFIERYGDC |
| 35148 | DSIIKDQSD |
| 35149 | LFIKVKDTS |
| 35150 | MAIQVFMSD |
| 35151 | ISIENEIMG |
| 35152 | APIQITKAY |
| 35153 | EKIRQNAAP |
| 35154 | ISICAQDID |
| 35155 | VGIMRNDTG |
| 35156 | GTIHMYCGW |
| 35157 | TRILNEFFC |
| 35158 | QNIRLGVPT |
| 35159 | DVIHTTHSG |
| 35160 | SHIQLNAWR |
| 35161 | ILIFIQNCP |
| 35162 | TRIVEPDAN |
| 35163 | ATIAQWCVH |
| 35164 | HVIGMGAGE |
| 35165 | RAIYNDMTE |
| 35166 | SAICQNYDA |
| 35167 | SQIQIRQAD |
| 35168 | NRIQMTDFQ |
| 35169 | FTIFPGNAG |
| 35170 | YVIGQKDEM |
| 35171 | IWITCPYAA |
| 35172 | NAIPYFVQA |
| 35173 | AVIHEKPHG |
| 35174 | ACIPQMHSA |
| 35175 | TSIEGLKTM |
| 35176 | ASIATMDRI |
| 35177 | PVICVWHDN |
| 35178 | CWILHHRCS |
| 35179 | VTWVHYDMY |
| 35180 | YTMVAPDYE |
| 35181 | SMPVGTKID |
| 35182 | MPKVHCASC |
| 35183 | WCLVNELPT |
| 35184 | VTSVAGGGE |
| 35185 | QQTVVQFCL |
| 35186 | ADVVMQTCC |
| 35187 | GVHVTESGA |
| 35188 | NYCVNKRAV |
| 35189 | MTSVCNWDS |
| 35190 | REKVWSDLK |
| 35191 | ICFVNLDHV |
| 35192 | MKHVSTMTK |
| 35193 | SGFVILEGI |
| 35194 | VFTVAHCVH |
| 35195 | WFNVRQNGQ |
| 35196 | QAPVIPIMA |
| 35197 | TNGVQIMHV |
| 35198 | INMVSMYAY |
| 35199 | QGFVVHSPS |
| 35200 | QFYVAEHLE |
| 35201 | RQCVIGDYD |
| 35202 | HTRVVKSTG |
| 35203 | GTAVSRQSK |
| 35204 | QTDVKVNFD |
| 35205 | NWTVHLIYG |
| 35206 | TAMVSECKD |
| 35207 | GHLVPFDCG |
| 35208 | HYVVRHEDP |
| 35209 | PTDVITTDQ |
| 35210 | CMEVTCFVV |
| 35211 | IQAVMASGW |
| 35212 | LESVKMDCN |
| 35213 | EMYVQRHSL |
| 35214 | AAFVMNGDG |
| 35215 | HCWVNEKCE |
| 35216 | SKDVYGMYC |
| 35217 | EDLVVYMGS |
| 35218 | VGNVMIKMC |
| 35219 | IHRVVCRDH |
| 35220 | MFVVNQNWA |
| 35221 | HEWVLDFFP |
| 35222 | RVEAYAPTT |
| 35223 | THCHYQGND |
| 35224 | WAGCYTYAN |
| 35225 | WDHNYFTYL |
| 35226 | IVMEYAFIP |
| 35227 | NSCTYNKDA |
| 35228 | FKNWYINMH |
| 35229 | ETNSYNSHH |
| 35230 | LSSCYYMTY |
| 35231 | GSTQYPCCK |
| 35232 | QAEGYRDCY |
| 35233 | IGQWYECMT |
| 35234 | GNAGYQADL |
| 35235 | GCMPYQINV |
| 35236 | SLCIYDHRH |
| 35237 | ETNHYLISD |
| 35238 | EGSDYVGYN |
| 35239 | NMMQYQWAI |
| 35240 | LPKIYLSFC |
| 35241 | QNLMYCSSV |
| 35242 | RPVTYPHTA |
| 35243 | FDKTYFHDC |
| 35244 | LMEHYLREG |
| 35245 | NVWYYFRTW |
| 35246 | WPSFYANYR |
| 35247 | IACKYERVC |
| 35248 | LMEHYLREW |
| 35249 | SMHDYNFNV |
| 35250 | GYRNYFIWP |
| 35251 | FAMSYQRQN |
| 35252 | EVYCYQLQS |
| 35253 | YKNSYSQWP |
| 35254 | SHMSYGQVQ |
| 35255 | IQCKYIHFT |
| 35256 | ANHLYHAAN |
| 35257 | NYHNYMFGM |
| 35258 | CFNTYDQCF |
| 35259 | SDNMYHTIE |
| 35260 | RPVHYQGTN |
| 35261 | MEHFYDRES |
| 35262 | PNGHYWMIP |
| 35263 | ATNKYANAI |
| 35264 | DKTCYLTCP |
| 35265 | TMSNYGCLQ |
| 35266 | MGMAYSDSE |
| 35267 | ELPRYYGQM |
| 35268 | DKYLYMYSD |
| 35269 | LLAKYMVHT |
| 35270 | MTERYYWCQ |
| 35271 | DFMWYHICS |
| 35272 | HGSSYNTVQ |
| 35273 | SLTQYQWRG |
| 35274 | TVNWYSGFG |
| 35275 | MEPHYWEWP |
| 35276 | STTAYHQDY |
| 35277 | VVTQYVDAP |
| 35278 | AVDHYSNGA |
| 35279 | NGNHYNAMR |
| 35280 | LHKAYVTDS |
| 35281 | SGNMYESGE |
| 35282 | CSESYRGPD |
| 35283 | YLEMYSKDW |
| 35284 | DPGWYGLAP |
| 35285 | MNSFYRAEW |
| 35286 | NEDQYMHVE |
| 35287 | WHGFYKIMF |
| 35288 | YTWPYPETH |
| 35289 | PGGCYRMME |
| 35290 | MYLNYRHFA |
| 35291 | WVHPYSYCA |
| 35292 | DYFQYNEAP |
| 35293 | RQTTYDCLD |
| 35294 | AVEDYWRYF |
| 35295 | TSCCYESSS |
| 35296 | EPRLYCAQE |
| 35297 | HTLLWYGIC |
| 35298 | MDHPTYGIY |
| 35299 | DGCQKYQEQ |
| 35300 | FKFISYVGD |
| 35301 | IYLKFYFSL |
| 35302 | DCDMKYLYE |
| 35303 | LGSPFYECG |
| 35304 | LLLNSYKWY |
| 35305 | RLSLWYFCH |
| 35306 | MDRMMYMLG |
| 35307 | AMGYQYLYE |
| 35308 | SVMLDYHDM |
| 35309 | IVKQCYDCH |
| 35310 | QVEDGYWSY |
| 35311 | EWLSLYCHW |
| 35312 | CTEIRYYGT |
| 35313 | CLHHHYPVT |
| 35314 | QYVGMYTQQ |
| 35315 | QHSRAYMWY |
| 35316 | TSSDHYALP |
| 35317 | YAMPLYFCE |
| 35318 | ECVSDYACC |
| 35319 | GHHQCYKAD |
| 35320 | VSLQKYDMT |
| 35321 | NQQRTYNNG |
| 35322 | QEYHIYFPF |
| 35323 | AEDTPYTIY |
| 35324 | YSVQAYMAN |
| 35325 | QTAEWYCGA |
| 35326 | ECGCMYWGY |
| 35327 | RKWSCHRGH |
| 35328 | RQTDTHRAN |
| 35329 | NVDAHERYR |
| 35330 | SRENLGRYN |
| 35331 | WMPNQSRGW |
| 35332 | SATARFRDN |
| 35333 | EKRLFDRTY |
| 35334 | LGHHKGRWH |
| 35335 | YEWPWQRGL |
| 35336 | YEWPWQRWL |
| 35337 | YEWPWRRWL |
| 35338 | GCLGTKRQI |
| 35339 | HDSLFKRTE |
| 35340 | HEWPWQRWL |
| 35341 | SYLRQQRDT |
| 35342 | ALANKRRNH |
| 35343 | HEGLIMRLE |
| 35344 | AQRCFHRWG |
| 35345 | LFCPTCRYA |
| 35346 | HLYSDGRGS |
| 35347 | QCKTHPRSF |
| 35348 | LFTDTKRPE |
| 35349 | EFTHAIRQC |
| 35350 | GATWLFRFP |
| 35351 | ASWLCGRNV |
| 35352 | AMLOKKRQD |
| 35353 | DWYLSSRDQ |
| 35354 | LHSYQWRLG |
| 35355 | CGHLLRRAE |
| 35356 | IEFTHSRKV |
| 35357 | PWYNWDRRC |
| 35358 | LCTSTERQG |
| 35359 | LVDRSGRTA |
| 35360 | NLDMSRRDF |
| 35361 | NAMCFGRAK |
| 35362 | VHHKTNRSY |
| 35363 | TVNRAMREE |
| 35364 | EAGPRKRDV |
| 35365 | CSVWMQRDR |
| 35366 | TFVYGERDR |
| 35367 | LREEELMTR |
| 35368 | WHRDEMLTT |
| 35369 | AMKPMILTN |
| 35370 | RAAGIQGTA |
| 35371 | EQELKNSTQ |
| 35372 | MATYSDPTS |
| 35373 | NPPHRNCTI |
| 35374 | SVTPDSITT |
| 35375 | EACHPDLTP |
| 35376 | ASEHKANTT |
| 35377 | AYPLENDTD |
| 35378 | TYYNMWHTK |
| 35379 | DDYQQHWTG |
| 35380 | DTLDMPETN |
| 35381 | NEQCHAKTS |
| 35382 | NEQCHVKTS |
| 35383 | PYKIVMITR |
| 35384 | IKHITHNTY |
| 35385 | STNPNTETR |
| 35386 | HMALVMQTA |
| 35387 | YDLWGWSTR |
| 35388 | IYMSTGNTP |
| 35389 | QQTKNNSTD |
| 35390 | DTFSNQNTV |
| 35391 | SAPRNEITR |
| 35392 | GSKIIMHTE |
| 35393 | MTKMMCETI |
| 35394 | WCHITWPTN |
| 35395 | CVGYKSNTP |
| 35396 | NCDHRFGTL |
| 35397 | SSQHASHTD |
| 35398 | IMTMQMDTA |
| 35399 | IKHITRNTY |
| 35400 | QFMMGQQTP |
| 35401 | MAMWIADTH |
| 35402 | RKVAVESTW |
| 35403 | IGQHNVQTT |
| 35404 | MLMHADMTQ |
| 35405 | MQPMESATW |
| 35406 | HRVHSEKTD |
| 35407 | RFDQFWDTY |
| 35408 | QSRCVDNTV |
| 35409 | FNESGWPTW |
| 35410 | ESSQEIKTC |
| 35411 | DQEAQVVTG |
| 35412 | TCKNLTYYI |
| 35413 | ECLLFLVGI |
| 35414 | EMKCIGAGI |
| 35415 | QPHEAEFKI |
| 35416 | YTNFEDVLI |
| 35417 | PPHIGTQFI |
| 35418 | WDNWSNTQI |
| 35419 | WVTLCADLI |
| 35420 | RSLDIQYDI |
| 35421 | RSFYHPKLI |
| 35422 | DHFYMSYGI |
| 35423 | VEQMVWADI |
| 35424 | CPWFIKWGI |
| 35425 | PHVSGEGKI |
| 35426 | QAWPKTEYI |
| 35427 | FNWFVRAYI |
| 35428 | DMGMDNKGI |
| 35429 | GTMCREQMI |
| 35430 | TSVIRLFWI |
| 35431 | YMHFRQYPI |
| 35432 | EFVLGCQGI |
| 35433 | WPNIKHQPI |
| 35434 | AAMESCAEI |
| 35435 | HQCQSRSYI |
| 35436 | YSDPKNMSI |
| 35437 | FFRICGSYI |

### Example 11

AAV5 Variants with Tissue Tropism in Mammary Gland

This example describes engineered AAV5 variants with tissue tropism in mammary gland that were discovered using the methods and systems described in EXAMPLE 1-EXAMPLE 2.

CNS, liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive mammary gland tropism. The results are shown in FIG. 18I which shows the positional frequency of each amino acid in the 581 to 589 region of AAV5 VP1 in mammary gland tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in mammary gland over the frequency of that given amino acid residue occurring at the specified position in variants identified in all other harvested tissues was analyzed to identify a set of sequence rules for capsids that preferentially target mammary gland tissue. With reference to TABLE 21 below, and SEQ ID NO: 2 (AAV5 VP1), the following amino acids can, thus, be independently mutated, in any combination, at any one or more positions Xaa1-Xaa9, to provide a VP1 capsid with enhanced mammary gland tropism as compared wildtype AAV5 VP1 capsid (SEQ ID NO: 1), where mammary gland tropism here refers to properties that are preferred for mammary gland transduction over properties that are preferred for transduction of all other harvested tissues.

TABLE 21
Mammary Gland Tropism Rules

Xaa1 is selected from C, K, M, Q, R, or Y
Xaa1 is selected from C, Q, or R
Xaa1 is C
Xaa2 is selected from A, F, I, K, S, T, or V
Xaa2 is selected from A, S, or V
Xaa2 is V
Xaa3 is selected from A, F, G, I, K, L, R, T, or Y
Xaa3 is selected from F, G, K, R, or Y
Xaa3 is selected from F, K, or Y
Xaa3 is F
Xaa4 is selected from A, I, K, Q, R, or T
Xaa4 is selected from A, I, or R
Xaa4 is I
Xaa5 is selected from I, L, M, Q, R, T, V, or Y
Xaa5 is selected from I, M, or Y
Xaa5 is Y
Xaa6 is selected from H, N, S, or V
Xaa6 is H
Xaa7 is selected from A, H, I, N, S or Y
Xaa7 is N or S
Xaa7 is N
Xaa8 is selected from A, C, D, G, H, M, Q, or S
Xaa8 is selected from G, M, or Q
Xaa8 is G
Xaa9 is selected from A, E, L, W, or Y
Xaa9 is selected from A, L, or W
Xaa9 is A

TABLE 22 below provides sequences of the 581 to 589 region for variant AAV VP1 capsid polypeptides that were found in mammary gland tissue and comport to one or more of the rules provided in TABLE 21. The present disclosure, thus, provides for rAAVs composed of engineered AAV5 VP1 capsid polypeptides having a SEQ ID NO: 2, wherein the Xaa1 to Xaa9 region is any one of SEQ ID NO: 22118-SEQ ID NO: 23117, as disclosed in TABLE 22. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 22
Sequences of the 581 to 589 Region in AAV5
VP1 Capsid Polypeptide that Drive Mammary
Gland Tissue Tropism

| SEQ | |
|---|---|
| ID | 581-589 |
| NO | Sequence |
| 22118 | CIKTDWQRI |
| 22119 | CAVSGPECY |
| 22120 | CCEIWLASS |
| 22121 | CDYWLVVPW |
| 22122 | CFTQKESAY |
| 22123 | CGYWLAVPW |
| 22124 | CIPTYHHFG |
| 22125 | CIRGQTHYN |
| 22126 | CNTPYEVWA |
| 22127 | CQSTGAHME |
| 22128 | CQSTGSHMA |
| 22129 | CSKATGGAE |
| 22130 | CSKTTWGAE |
| 22131 | CYNSGDCGN |
| 22132 | CIMSHCRQH |
| 22133 | CSNKTCCKA |
| 22134 | CGYWLVVPW |
| 22135 | CHMLNDCMS |
| 22136 | CSKATWGAE |
| 22137 | CKNPSWYMD |
| 22138 | CFEMYYAMW |
| 22139 | CDPCMECGS |
| 22140 | CTDGKHASW |
| 22141 | CFCTYKIEH |
| 22142 | CIGPHWHIP |
| 22143 | CAHQQHNED |
| 22144 | CAHSYQMSR |
| 22145 | CAIHIFLQD |
| 22146 | CAIQISDTS |
| 22147 | CALSALTME |
| 22148 | CALVSRSQD |
| 22149 | CAMIHGKYD |
| 22150 | CANFAMQQF |
| 22151 | CASRSMLTE |
| 22152 | CAVRTFMGS |
| 22154 | CCGIFNGIY |
| 22155 | CCHMQYRES |
| 22156 | CEMYREYFI |
| 22157 | CFAKMEKLQ |
| 22158 | CFAPYMNVD |
| 22159 | CFMHYGRIA |
| 22160 | CFSQFASGS |
| 22161 | CFYQKGAAY |
| 22162 | CGFWLDQNE |
| 22163 | CHRLVMGYH |
| 22164 | CIAPRPEWP |
| 22165 | CIKLKCSHS |
| 22166 | CILWKHAMP |
| 22167 | CISNTRFGV |
| 22168 | CITMPMNFG |
| 22169 | CIYIWFTRD |
| 22170 | CKSGMVHHA |
| 22171 | CKSMGLFLV |
| 22172 | CKYDYHQNT |
| 22173 | CLGYMWQNT |
| 22174 | CMKADGMQR |
| 22175 | CMSDRGLQS |
| 22176 | CMYMQNAMG |
| 22177 | CNNLSVCDE |
| 22178 | CNSLFTNQQ |
| 22179 | CPTMYKMQA |
| 22180 | CQKCYDHYQ |
| 22181 | CQYRTRMIT |
| 22182 | CSAPTLSGN |
| 22183 | CSFHIIMGE |
| 22184 | CSFHNNEHA |
| 22185 | CSICEHCMD |
| 22186 | CSMAEQKSQ |
| 22187 | CSMRYMAVH |
| 22188 | CSRPLHDSY |
| 22189 | CSTQYMKHM |
| 22190 | CSVNFYTSF |
| 22191 | CSWEWFRAE |
| 22192 | CSWQLWQSC |
| 22193 | CSYSTQIGH |
| 22194 | CTAITLRYP |
| 22195 | CTERTESNR |
| 22196 | CTHDIWAQA |
| 22197 | CTHQIHLPA |
| 22198 | CTKTYMHDF |
| 22199 | CTLNVQSDG |
| 22200 | CTLQWSAGP |
| 22201 | CTMRVQMKP |
| 22202 | CTVLRFDHQ |
| 22203 | CVAYSYNST |
| 22204 | CVCGKKQMG |
| 22205 | CVFAQSGSY |
| 22206 | CVFYQRKVL |
| 22207 | CVIAKYYQE |
| 22208 | CVIHTPYQY |
| 22209 | CVIRDGHYD |
| 22210 | CVKMKQDFN |
| 22211 | CVMEQQKER |
| 22212 | CVNTTHDYQ |
| 22213 | CVNVMAADR |
| 22214 | CVQITLDGF |
| 22215 | CVRMCVDHA |
| 22216 | CVSMIVPMV |
| 22217 | CVTCKFDEQ |
| 22218 | CVTLTHRDK |
| 22219 | CVTYSSAPK |
| 22220 | CVYPTMKAD |
| 22221 | CWICMCVSP |
| 22222 | CWKFRDHDF |
| 22223 | CWLHTRTHW |
| 22224 | CWTTANTSN |
| 22225 | CYAKVTANT |
| 22226 | CYEQWNGLA |
| 22227 | CYFLHNNHE |
| 22228 | CYYTIEKAE |
| 22229 | CAVGGPECY |
| 22230 | CKTQERANS |
| 22231 | CYNSGDCDN |
| 22232 | CTSTDPIAP |
| 22233 | CRGKSHDHT |
| 22234 | CPALNYDCV |
| 22235 | CMLTHIMEF |
| 22236 | CTFPSADMC |
| 22237 | CQSTGSHME |
| 22238 | CSFHMNITS |
| 22239 | CIPTYRHFG |
| 22240 | CYGRVAHHE |
| 22241 | CTEIRYYGT |
| 22242 | CVKPFLTGW |
| 22243 | CAWYMKQGN |
| 22244 | CAEPVYMWK |
| 22245 | CAGTSMAKE |
| 22246 | CASHVKWGP |
| 22247 | CAVAYMSCT |
| 22248 | CAYKPHVAP |
| 22249 | CCHNEHRHM |
| 22250 | CCVSFAYSC |
| 22251 | CDAVHNDAS |
| 22252 | CEYLPDMHK |
| 22253 | CFHWVNHMT |
| 22254 | CFQTANIGG |
| 22255 | CHDAQFKTD |
| 22256 | CHKSWWRLD |
| 22257 | CHYWVSTDG |
| 22258 | CIAHRHAQP |
| 22259 | CIFRLSALS |
| 22260 | CIGSRQAIA |
| 22261 | CIHIASRLG |
| 22262 | CISFYTSAG |
| 22263 | CITKLMVNA |
| 22264 | CIVTDSMAP |
| 22265 | CIYYPNFYN |
| 22266 | CKQMYLATE |
| 22267 | CKVPVFSLE |
| 22268 | CLRVCNCSP |
| 22269 | CLRVYNCSP |
| 22270 | CMAISPSFE |
| 22271 | CMLRYMLGR |
| 22272 | CMPQKYHKD |
| 22273 | CMSAMCMWS |
| 22274 | CMSKIVWSS |
| 22275 | CNDQAQYDW |
| 22276 | CNTSMAIMG |
| 22277 | CPWWSDNCM |
| 22278 | CRVTITYPT |
| 22279 | CSFQMQFMG |
| 22280 | CSFVLDNAF |
| 22281 | CSGPVSASA |
| 22282 | CSKYFNDWG |
| 22283 | CSKYSAEYP |
| 22284 | CSTPHHLQL |
| 22285 | CSVNIGQHA |
| 22286 | CSYKDQSQQ |
| 22287 | CSYTHWRSA |
| 22288 | CTAHPMLQA |
| 22289 | CTATKILHA |
| 22290 | CTFVALSMQ |
| 22291 | CTGSIVKQM |
| 22292 | CTHPRITGA |
| 22293 | CTIKQLCNG |
| 22294 | CVANCQQCI |
| 22295 | CVARNINAN |
| 22296 | CVCMYQNMQ |
| 22297 | CVFLAQKMK |
| 22298 | CVFMRSHDK |
| 22299 | CVFPVHSSN |
| 22300 | CVHKICMHH |
| 22301 | CVHQNSRAK |
| 22302 | CVHRGLTYT |
| 22303 | CVHVAMHYP |
| 22304 | CVHYANMML |
| 22305 | CVISKMWCH |
| 22306 | CVLRSNVQL |
| 22307 | CVNEFFTCF |
| 22308 | CVNMKTDGN |
| 22309 | CVNQLCDSQ |
| 22310 | CVNVENLTA |
| 22311 | CVPDTIDTE |
| 22312 | CVQMIMKDW |
| 22313 | CVQTDNRRP |
| 22314 | CVRHYGEYW |
| 22315 | CVVADQGRG |
| 22316 | CVWHAPIQT |
| 22317 | CVWHASIQT |
| 22318 | CVYTKIIGH |
| 22319 | CWEFMFWHG |
| 22320 | CWGVISKAP |
| 22321 | CWIRGTQDR |
| 22322 | CYLITFREH |
| 22323 | TVADSSRLM |
| 22324 | TVCMFQVLP |
| 22325 | TVFPMTVVP |
| 22326 | TVGFSEQDA |
| 22327 | TVIQWYWHH |
| 22328 | TVLSWGLCN |
| 22329 | TVNQECNCQ |
| 22330 | TVRPQDNKN |
| 22331 | VVDHSCSRV |
| 22332 | VVDPFGHEQ |
| 22333 | VVHENQLKP |
| 22334 | WVTCRGTVF |
| 22335 | YVEIHHAQK |
| 22336 | YVHEGGENP |
| 22337 | YVLESNYCQ |
| 22338 | YVSASWMTP |
| 22339 | YVTKFAECH |
| 22340 | YVVTHVPWH |
| 22341 | YVYDEFLWS |
| 22342 | FVMCYNDCK |
| 22343 | AVHWHRKWE |
| 22344 | AVINPPCHP |
| 22345 | AVSLQPQFG |
| 22346 | DVKAFMGCV |
| 22347 | GVEAYRNFK |
| 22348 | GVIHRQYCP |
| 22349 | KVEANPRVC |
| 22350 | LVHAVSYIC |
| 22351 | MVAPPNCCV |
| 22352 | NVNVRESEW |
| 22353 | PVHAVSYIC |
| 22354 | RVTIMFTDT |
| 22355 | SVHTADVCC |
| 22356 | SVPQQKIDE |
| 22357 | TVKINSISP |
| 22358 | YVQCTHYAQ |
| 22359 | GVIHRQYCQ |
| 22360 | QVPRPSMWW |
| 22361 | PVSPLRSYV |
| 22362 | QVTSDGKII |
| 22363 | VVGWDNYVA |
| 22364 | NVHHEWCDW |
| 22365 | MVDPMSVPF |
| 22366 | DVDYRSEAW |
| 22367 | IVPQQKIDE |
| 22368 | VVHPMAYCF |
| 22369 | LVVTKFTYV |
| 22370 | AVKSRTTWV |
| 22371 | AVQGDPGVA |
| 22372 | AVSVETWWT |
| 22373 | AVVDCPLNI |
| 22374 | AVWNVVSDG |
| 22375 | AVYQYVETP |
| 22376 | DVDKLEHGQ |
| 22377 | DVDNCYKGD |
| 22378 | DVICFGKWE |
| 22379 | DVQTRNLSK |
| 22380 | DVYRDAMTP |
| 22381 | EVALQWAST |
| 22382 | EVDVRVLFD |
| 22383 | EVESWMWVC |
| 22384 | EVHRVASCP |
| 22385 | EVSKFNHYF |
| 22386 | EVSTITFWS |
| 22387 | EVTPHWTGG |
| 22388 | EVYYAMASW |
| 22389 | FVNVMKVTC |
| 22390 | GVKPGASCG |
| 22391 | GVQQFYRYD |
| 22392 | GVRLSSPWE |
| 22393 | GVYPMHEAC |
| 22394 | HVCMMASCD |
| 22395 | IVCAYSNAS |
| 22396 | IVEPAIVCA |
| 22397 | IVNKWDGFA |
| 22398 | IVYYCGKRK |
| 22399 | KVICHEFTS |
| 22400 | KVMHTGWHP |
| 22401 | KVVQRIDHD |
| 22402 | LVCRYWDDE |
| 22403 | LVFMQEYAA |
| 22404 | LVKCNTCME |
| 22405 | LVNHCGQMG |
| 22406 | LVTEQVNSP |
| 22407 | MVDRMKWHK |
| 22408 | MVYPIEWGA |
| 22409 | NVGIITFQD |
| 22410 | NVLQNSIMC |
| 22411 | NVYTHHVLS |
| 22412 | QVDNYFNRL |
| 22413 | QVNFGLHAQ |
| 22414 | RVNEATHDD |
| 22415 | SVCPFSYNL |
| 22416 | SVFSNQDGV |
| 22417 | SVGFEQSWY |
| 22418 | SVIPLPHGD |
| 22419 | SVITTEFQD |
| 22420 | SVVNHAGAC |
| 22421 | TVAHERSFQ |
| 22422 | TVDNTNWLK |
| 22423 | TVHYSSQDP |
| 22424 | TVSGLWQEL |
| 22425 | VVGTYALTY |
| 22426 | VVHTCAMCT |
| 22427 | VVSAHMPGV |
| 22428 | VVTVAWTPG |
| 22429 | WVEKYLISY |
| 22430 | YVLSAIPFG |
| 22431 | YVWYRRTDL |
| 22432 | KVCNSEYWD |
| 22433 | QVHIKVTDH |
| 22434 | KVDRNTNWC |
| 22435 | GVEAYCNFK |
| 22436 | HVWNDVFHM |
| 22437 | EVQRNIYSP |
| 22438 | GVAQYLHHC |
| 22439 | DVPIHRVQL |
| 22440 | SVNLNCTEH |
| 22441 | FVFAMIPGF |
| 22442 | AVHWHRKCE |
| 22443 | GVMIYEADE |
| 22444 | KVAGGHETT |
| 22445 | FVPWCHSFG |
| 22446 | NVTPGRTHS |
| 22447 | LVSQWDYDM |
| 22448 | RVDIGGFAQ |
| 22449 | HVDQHRVQA |
| 22450 | AVEACYHSP |
| 22451 | AVFTFATCG |
| 22452 | AVQWSAENM |
| 22453 | DVDYRPDAQ |
| 22454 | DVELGGSNF |
| 22455 | DVFQYEMLA |
| 22456 | DVGYASDPK |
| 22457 | DVKVTEDAE |
| 22458 | DVRLDDGAQ |
| 22459 | DVRPDEFHN |
| 22460 | DVRPDKFHN |
| 22461 | DVVSTESTT |
| 22462 | EVFREWMWM |
| 22463 | EVGSDCHQG |
| 22464 | EVKYMRHIC |
| 22465 | EVRPSVGSD |
| 22466 | EVTSAGFDF |
| 22467 | FVIWRQESR |
| 22468 | FVRCLSTHN |
| 22469 | FVSEQMTAQ |
| 22470 | GVDSSRPVQ |
| 22471 | HVCQKTHSI |
| 22472 | HVNTSCKQF |
| 22473 | HVQHHDAAT |
| 22474 | IVAYGTCGA |
| 22475 | IVHQKQLAA |
| 22476 | LVDEMFSQW |
| 22477 | LVKGSFAPD |
| 22478 | LVKPQSGEL |
| 22479 | LVRQDSQIG |
| 22480 | LVVCSCPFP |
| 22481 | MVEFYCSDE |
| 22482 | MVEFYYSDE |
| 22483 | NVAYHMVNN |
| 22484 | NVENAYDAM |
| 22485 | NVFLNLDLA |
| 22486 | NVHTIENSA |
| 22487 | NVVDILAGS |
| 22488 | NVYYKHAHP |
| 22489 | PVIRTWVDI |
| 22490 | QVASHTQPE |
| 22491 | QVATWSMYH |
| 22492 | QVFMASTGE |
| 22493 | RVGVQEAVC |
| 22494 | RVHISLLKG |
| 22495 | RVNMKTDGN |
| 22496 | SVCLHNLVG |
| 22497 | SVHGFDQSM |
| 22498 | SVIKVAFFF |
| 22499 | SVRLHNLVG |
| 22500 | TVDPNVRVD |
| 22501 | TVVPQGFDR |
| 22502 | YVCIPAHDR |
| 22503 | YVCMIHMDD |
| 22504 | YVDLIMSVD |
| 22505 | YVISKCAGQ |
| 22506 | TSFCQIASR |
| 22507 | VCFKLFHVE |
| 22508 | AFFKFMTQM |
| 22509 | DKFPMPTHS |
| 22510 | ETFGIRTFA |
| 22511 | GIFWASYGF |
| 22512 | GKFPMPTHS |
| 22513 | GMFCASYGF |
| 22514 | KDFWGHQCF |
| 22515 | LLFTLMSSS |
| 22516 | QTFYEGHAT |
| 22517 | RDFRMNAGA |
| 22518 | RTFYEGNAT |
| 22519 | TTFQSLQQM |
| 22520 | NQFHWVRYD |
| 22521 | LWFFMKFAK |
| 22522 | YTFWDYHRS |
| 22523 | KDFWEHQCF |
| 22524 | VHFTVLNWG |
| 22525 | DYFQYNEAP |
| 22526 | QEFLEERYV |
| 22527 | GTFHCCAWE |
| 22528 | ETFGIRTFD |
| 22529 | EQFADRAPR |
| 22530 | ARFMWNHTR |
| 22531 | DTFLEMSMG |
| 22532 | DTFPIAGSF |
| 22533 | EAFRHDHFH |
| 22534 | EGFFYHHGG |
| 22535 | EGFQFSNQG |
| 22536 | ERFMTASMQ |
| 22537 | FEFTVHCTH |
| 22538 | GKFEPPVNA |
| 22539 | HAFPTVRWG |
| 22540 | IMFDPNIPN |
| 22541 | KAFHIQREK |
| 22542 | LDFQIRFER |
| 22543 | LPFHLQSDT |
| 22544 | LPFTQMHGS |
| 22545 | LSFPVVLTE |
| 22546 | NAFEYEKIM |
| 22547 | NAFTKTQDQ |
| 22548 | NSFSTNWSY |
| 22549 | NTFCETVTF |
| 22550 | QKFEGEWFT |
| 22551 | RAFVGCSPE |
| 22552 | RQFAHRFKM |
| 22553 | SCFAILSAN |
| 22554 | TSFELNDER |
| 22555 | TSFSPELFN |
| 22556 | WNFNCIILH |
| 22557 | STFISSGIQ |
| 22558 | FKFWDMETR |
| 22559 | KRFVDMPEM |
| 22560 | VSFQRMNDM |
| 22561 | ERFKTFCEA |
| 22562 | LLFPLMSSS |
| 22563 | EKFWFKTMN |
| 22564 | NWFQAIEIG |
| 22565 | GIFCASYGF |
| 22566 | IWFWWEKAE |
| 22567 | RTFYEGHAT |
| 22568 | TTFPQVIMN |
| 22569 | DAFINRLSG |
| 22570 | DIFDRPSSD |
| 22571 | ETFLPWVHT |
| 22572 | ETFSKTWFG |
| 22573 | KSFGSPFER |
| 22574 | KSFGSPFEW |
| 22575 | MAFYKSQFN |
| 22576 | NAFGMEYNT |
| 22577 | NSFKPSGVT |
| 22578 | PWFMVIWWP |
| 22579 | QCFFNHRCE |
| 22580 | RAFQDHYAY |
| 22581 | TIFSASDSK |
| 22582 | VAFYQENTT |
| 22583 | WHFSSKWMD |
| 22584 | TPGITQQQW |
| 22585 | TSHICVSSL |
| 22586 | TWLIDADMA |
| 22587 | VAMIMEGQF |
| 22588 | VFKIREDDN |
| 22589 | WADIEYSHD |
| 22590 | WTGIKCQSS |
| 22591 | YFCILEKMT |
| 22592 | YMCIQMAGW |
| 22593 | ESPIMLNQA |
| 22594 | RTPIELMHG |
| 22595 | AGQITTSVT |
| 22596 | ESDIFDGRQ |
| 22597 | HPSICKGGD |
| 22598 | QCHIIVARE |
| 22599 | QCHIIVARG |
| 22600 | TAKIIMLAS |
| 22601 | DMSIATWYP |
| 22602 | ESDIFDGHQ |
| 22603 | TCVIGKGRR |
| 22604 | VACIEHAKC |
| 22605 | QFDIDQCCT |
| 22606 | HPSICQGGD |
| 22607 | AWIISQEGR |
| 22608 | DMWILKDAS |
| 22609 | DYYIIDCFM |
| 22610 | EHMIEFPHR |
| 22611 | FREIKEYDM |
| 22612 | GLYITHSME |
| 22613 | HCNIFSREQ |
| 22614 | IICICNRGH |
| 22615 | KEVIMKRCV |
| 22616 | KLEIMCQAK |
| 22617 | KPGIMKFYN |
| 22618 | LYNIPMGTT |
| 22619 | MKDIRVTQR |
| 22620 | MYTIGTCYG |
| 22621 | NINIYWMID |
| 22622 | NSAIMSNYM |
| 22623 | QEDILQTQY |
| 22624 | QFEICHSQE |
| 22625 | QHLIMPEPH |
| 22626 | QPLIQHAGG |
| 22627 | QQTIDSYGH |
| 22628 | QSGINDLYG |
| 22629 | RCMIYHQNA |
| 22630 | RGGIFPQRP |
| 22631 | RMCIDYLRP |
| 22632 | TACIQQKLL |
| 22633 | TNRIVTRII |
| 22634 | TQQILHDAF |
| 22635 | TRIINVILG |
| 22636 | TSLITGQLY |
| 22637 | VEPIMFWPA |
| 22638 | VNYIICKMD |
| 22639 | VNYIICKMG |
| 22640 | IKHITHNTY |
| 22641 | MYAIADELD |
| 22642 | NPSIQPKTM |
| 22643 | PENIAEDHE |
| 22644 | MRKIKIACM |
| 22645 | LRAILDCGW |
| 22646 | EIAIDQDIH |
| 22647 | SRTICVHGN |
| 22648 | IANITACAN |
| 22649 | EISIRLLDE |
| 22650 | EIYILPKFP |
| 22651 | GSAIFVKEE |
| 22652 | GYTIKNGYN |
| 22653 | HHEIANLHQ |
| 22654 | KHQINAVER |
| 22655 | NGEIFSCMS |
| 22656 | NGEIFSYMS |
| 22657 | QFKIYDCVG |
| 22658 | QIGITYYLP |
| 22659 | QMGINHDDR |
| 22660 | QPAINTQCA |
| 22661 | SFKILNRGG |
| 22662 | SPDIDLINH |
| 22663 | STSITELAM |
| 22664 | TTLIKMHVE |
| 22665 | TWEIKHNQV |
| 22666 | VACIANLWT |
| 22667 | VFAIMTRTQ |
| 22668 | WSNIYWRQD |
| 22669 | THGHYTMSA |
| 22670 | TRAGYNEQD |
| 22671 | TSVKYSAWG |
| 22672 | TYWNYAEAK |
| 22673 | VFTDYWEQG |
| 22674 | VFTDYWERG |
| 22675 | VIDWYPHQQ |
| 22676 | VSDEYVMEQ |
| 22677 | WELTYDCLA |
| 22678 | WIICYIALQ |
| 22679 | WLIVYDHGN |
| 22680 | YAHSYGHFD |
| 22681 | MAEQYKYGH |
| 22682 | TGAKYHDWL |
| 22683 | EAENYFPRH |
| 22684 | GLMDYSLCA |
| 22685 | KEIQYHSYE |
| 22686 | MDTCYMYAD |
| 22687 | QPPKYFVHQ |
| 22688 | RNTPYEFWA |
| 22689 | SGNMYESGA |
| 22690 | TEPHYWEWP |
| 22691 | TTERYYWCQ |
| 22692 | KEIPYHSYE |
| 22693 | LMAWYENWY |
| 22694 | ITRTYQEMQ |
| 22695 | VREYYPKFH |
| 22696 | QQEAYILRI |
| 22697 | DMTFYLSRD |
| 22698 | MMPEYFNKD |
| 22699 | FITAYHPCL |
| 22700 | GQKSYDGCH |
| 22701 | MFEKYFQTM |
| 22702 | NEAPYSTWQ |
| 22703 | SMSEYHYDE |
| 22704 | AAVQYSLMG |
| 22705 | AEEVYGYHQ |
| 22706 | AQMEYDCAM |
| 22707 | DDALYAVFY |
| 22708 | DSWMYHVQL |
| 22709 | DYIHYKNGM |
| 22710 | EFSYYHTHG |
| 22711 | ESEKYTWSG |
| 22712 | FGATYNQTI |
| 22713 | FGLWYRDHK |
| 22714 | FHITYLMDK |
| 22715 | GAQQYADSI |
| 22716 | GFCHYWKQG |
| 22717 | GNCKYHVTQ |
| 22718 | HIVTYEHHE |
| 22719 | HPCYYSWAP |
| 22720 | HWDNYWKMD |
| 22721 | HWREYPSKG |
| 22722 | ICHQYTRHG |
| 22723 | IPLAYPEYQ |
| 22724 | ISKAYVDWW |
| 22725 | IYHPYDMLG |
| 22726 | KQMEYDNFH |
| 22727 | LMIQYVNKH |
| 22728 | LMREYMSVP |
| 22729 | MCQWYATNL |
| 22730 | MSDYYKEDS |
| 22731 | MYGGYAAGF |
| 22732 | NFCGYQAMH |
| 22733 | NGLQYMSSA |
| 22734 | NYHYYDHAG |
| 22735 | PTVAYENLD |
| 22736 | QAIGYNYFW |
| 22737 | QGQYYYECG |
| 22738 | QWPLYVMDR |
| 22739 | REELYQCAP |
| 22740 | REKNYELSE |
| 22741 | RWYWYESCN |
| 22742 | SMTSYYRQQ |
| 22743 | SWVWYPNLC |
| 22744 | WATAYWQGI |
| 22745 | WCGFYFLAA |
| 22746 | WNSPYKKRY |
| 22747 | YQVHYASHG |
| 22748 | KCTEYVHTC |
| 22749 | PIHTYKRHP |
| 22750 | RMKWYLPCV |
| 22751 | FAMSYQRQN |
| 22752 | EASWYYPAI |
| 22753 | MEPHYWEWP |
| 22754 | MSTDYCKVN |
| 22755 | TSKDYNEEI |
| 22756 | MTERYYWCQ |
| 22757 | QTPKYFVHQ |
| 22758 | YSKQYAEYY |
| 22759 | HSDWYMKYP |
| 22760 | AEGYYVFQP |
| 22761 | AWGCYQKGY |
| 22762 | DIKMYWEGA |
| 22763 | FGILYNNNE |
| 22764 | FNQLYMTFH |
| 22765 | GAEGYSKDQ |
| 22766 | GAEGYSKGQ |
| 22767 | GINDYKSGN |
| 22768 | GMDFYFNLE |
| 22769 | HKMAYGRDQ |
| 22770 | IAYEYWDYW |
| 22771 | IKEGYQLGG |
| 22772 | ISQFYLLDQ |
| 22773 | KECNYQARG |
| 22774 | KPLQYEAIQ |
| 22775 | LPNPYKTVV |
| 22776 | LPVQYEDRD |
| 22777 | MCDNYKSLA |
| 22778 | NHGNYEIAW |
| 22779 | NTMPYGHSY |
| 22780 | NYIPYGRLA |
| 22781 | QTQTYAYMD |
| 22782 | RFDHYNNEQ |
| 22783 | RKEYYPNVQ |
| 22784 | SAYQYLGCH |
| 22785 | SCGPYLWGT |
| 22786 | SHGNYEIAW |
| 22787 | SMNLYNRGV |
| 22788 | SMPPYRMLS |
| 22789 | SYQPYSKTV |
| 22790 | TANCYPILG |
| 22791 | TSWMYYDYG |
| 22792 | TYCLYGISE |
| 22793 | VALHYDHPN |
| 22794 | YFQLYMKEG |
| 22795 | YWAGYHMWP |
| 22796 | YYTCYPPSM |
| 22797 | TTHSDHHHA |
| 22798 | TYNLEHQCS |
| 22799 | TYRLEHECI |
| 22800 | VCQKVHGLF |
| 22801 | VLDVIHWHW |
| 22802 | VSEETHNFR |
| 22803 | VWNWEHKCT |
| 22804 | WYQCLHYQN |
| 22805 | YAWCQHCKN |
| 22806 | YHMDVHDSM |
| 22807 | YNMLTHVSM |
| 22808 | ETTLAHRWE |
| 22809 | HSAARHMFV |
| 22810 | LAECKHSSA |
| 22811 | MFMMMHDIW |
| 22812 | MFMTMHDIW |
| 22813 | NLNNWHTDM |
| 22814 | QNNWGHYNK |
| 22815 | QTLGGHMCT |
| 22816 | SSNFNHHIY |
| 22817 | MTAKEHAYL |
| 22818 | HQVLDHQLQ |
| 22819 | DMRRAHQEK |
| 22820 | VNQMMHTQA |
| 22821 | MFSFKHVHW |
| 22822 | AQASAHIMS |
| 22823 | DAIPRHICR |
| 22824 | DCSQQHMTL |
| 22825 | DEGVEHRPM |
| 22826 | DEMLQHSIM |
| 22827 | DFHNAHFGS |
| 22828 | DHDTNHWSH |
| 22829 | EASTFHRQG |
| 22830 | ECAETHFMK |
| 22831 | ECHVGHSGC |
| 22832 | EMCSVHQAW |
| 22833 | FCKFMHETH |
| 22834 | GFCQLHTQE |
| 22835 | GPSLIHQAA |
| 22836 | HYETEHDGC |
| 22837 | IAKASHKDR |
| 22838 | IASGTHHCQ |
| 22839 | IIEQSHTLE |
| 22840 | KLDLTHRMQ |
| 22841 | LFTKQHVYS |
| 22842 | LKHFVHAAS |
| 22843 | LTLGNHGEY |
| 22844 | NAGCQHEYE |
| 22845 | NORVEHAFS |
| 22846 | NTEREHRYN |
| 22847 | QIVHDHRHR |
| 22848 | RYDCMHCYA |
| 22849 | SAGEIHEHF |
| 22850 | SLDGVHDGF |
| 22851 | SLHYWHSAN |
| 22852 | SSNYWHASS |
| 22853 | STKAHHWCQ |
| 22854 | TEAATHWHR |
| 22855 | TEDLSHLWW |
| 22856 | TFQQSHAAV |
| 22857 | TISLVHLSE |
| 22858 | TMDVLHCDY |
| 22859 | TWCYRHNVE |
| 22860 | VSSQGHRFY |
| 22861 | VWMTWHASY |
| 22862 | YHAPQHHMG |
| 22863 | TQKGNHCRW |
| 22864 | APRNVHCGT |
| 22865 | LMEYCHDRF |
| 22866 | NYDFLHHVE |
| 22867 | NCIAKHEVT |
| 22868 | NSAHVHYFH |
| 22869 | NGYLVHACQ |
| 22870 | SDGMVHNCW |
| 22871 | KCGVEHEHR |
| 22872 | QATNEHIFW |
| 22873 | DTHHTHWQY |
| 22874 | FTQAIHHPD |
| 22875 | LEIVMHSLP |
| 22876 | GAKYRHYLQ |
| 22877 | APNFCHCSF |
| 22878 | ATHDMHRLC |
| 22879 | DDKCDHLQH |
| 22880 | DTKWMHQHC |
| 22881 | DTLDWHMAY |
| 22882 | EGVPLHRAF |
| 22883 | VSMFNQNSC |
| 22884 | WAHYTINQQ |
| 22885 | YHRHIANNG |
| 22886 | YLHNQGNGL |
| 22887 | YRGAACNPH |
| 22888 | DLGMINNME |
| 22889 | DEKAEKNES |
| 22890 | FASMQANDR |
| 22891 | GGHLEGNWQ |
| 22892 | HITMWYNGD |
| 22893 | HRQESPNDK |
| 22894 | NEIFNQNSY |
| 22895 | NGVALENRG |
| 22896 | NPWFLDNQF |
| 22897 | QMTSSTNCV |
| 22898 | TKQQLINSN |
| 22899 | NEISNQNSY |
| 22900 | VECTDTNDE |
| 22901 | HITVWYNGD |
| 22902 | RNNWKVNSQ |
| 22903 | YCCSDTNTE |
| 22904 | QMTSSTNCA |
| 22905 | PACQKGNHA |
| 22906 | ACLWORNDE |
| 22907 | ATVQFANDG |
| 22908 | DALTHGNFE |
| 22909 | DHAKLNNPS |
| 22910 | DITGHCNPA |
| 22911 | EAINRVNCQ |
| 22912 | EHEHRINQC |
| 22913 | EHRQLVNNN |
| 22914 | EKIPHYNME |
| 22915 | EKWDMENTE |
| 22916 | FTYCVNNYQ |
| 22917 | GKTCAENWI |
| 22918 | GYRPELNSA |
| 22919 | HFDVICNCY |
| 22920 | IMALDQNTN |
| 22921 | IMEPMNNAF |
| 22922 | IRMYAYNWL |
| 22923 | LESQQSNYT |
| 22924 | LIGPQANLY |
| 22925 | LTSMQENHS |
| 22926 | MIQYNKNMG |
| 22927 | MSEPRYNDL |
| 22928 | NCEWEMNDR |
| 22929 | NRSMFKNVR |
| 22930 | PTDFNENDC |
| 22931 | QFYSMDNHD |
| 22932 | QQEVKKNNA |
| 22933 | RNCMDTNGM |
| 22934 | RWADVTNMS |
| 22935 | STIYAFNWA |
| 22936 | TCMQCSNMP |
| 22937 | TWAPHNNGM |
| 22938 | VAKTSWNHC |
| 22939 | WICPKINAP |
| 22940 | WSILGTNWG |
| 22941 | YDPHNNNGV |
| 22942 | YSVARCNMD |
| 22943 | YSVCHMNAY |
| 22944 | YIRVGGNNE |
| 22945 | FQIRQDNLA |
| 22946 | KIDAHSNME |
| 22947 | NSVALENRG |
| 22948 | KNTMCDNDA |
| 22949 | ALKSWMNGS |
| 22950 | ILIFIQNCP |
| 22951 | QMKPLINDE |
| 22952 | TISHHLNRE |
| 22953 | DHLFNNNEW |
| 22954 | ETGYRENPC |
| 22955 | TSDTMWEGD |
| 22956 | TTVRKQFGY |
| 22957 | VGGSVNRGG |
| 22958 | VLYYSNYGH |
| 22959 | WAMFMYEGA |
| 22960 | YAQCFGQGI |
| 22961 | YAQCFSQGI |
| 22962 | YLRSLKCGN |
| 22963 | NGCTTTKGE |
| 22964 | DPKWEQYGM |
| 22965 | EFVMGCQGI |
| 22966 | FCQAINIGH |
| 22967 | GCMTRLDGL |
| 22968 | HFHEVAGGS |
| 22969 | HHMHIWLGL |
| 22970 | HTVGMAEGD |
| 22971 | IQAVMVSGW |
| 22972 | KSMPPGQGY |
| 22973 | SRDTWQIGR |
| 22974 | TIGVKRWGQ |
| 22975 | VLMFNRGGS |
| 22976 | VLMFNRWGS |
| 22977 | DPKWEKYGM |
| 22978 | TNHCRDAGT |
| 22979 | KWTDCIIGY |
| 22980 | DTDKQGQGI |
| 22981 | MTRQPEDGF |
| 22982 | ASDKMDVGS |
| 22983 | QPEQGGWGT |
| 22984 | KSMPPGQGD |
| 22985 | AACHFTYGS |
| 22986 | AATCSLKGH |
| 22987 | AHADMTTGF |
| 22988 | AMQRSVAGI |
| 22989 | AYEHCEHGP |
| 22990 | DIADRNVGH |
| 22991 | DRMCMMCGV |
| 22992 | DRSEFRQGT |
| 22993 | DTPDFNTGI |
| 22994 | ETEGQVAGA |
| 22995 | ETNLHTKGH |
| 22996 | HALSLTWGD |
| 22997 | HSLPHTIGL |
| 22998 | IHSRVNAGH |
| 22999 | IQKVTKRGA |
| 23000 | KEGPTPVGH |
| 23001 | KFDFWVAGH |
| 23002 | KHTQQDSGS |
| 23003 | LATHQKDGR |
| 23004 | LMELTNEGW |
| 23005 | LSPKNDPGV |
| 23006 | LSYSIVTGE |
| 23007 | MAVTLMHGC |
| 23008 | MCVRWSVGD |
| 23009 | MDTHLQHGA |
| 23010 | MGGSNVWGN |
| 23011 | MKWDCLIGP |
| 23012 | NFVCSDIGK |
| 23013 | NLMRRDHGG |
| 23014 | QAMYTTAGI |
| 23015 | QLKNAFEGS |
| 23016 | QQYAPVIGP |
| 23017 | QSREPRGGK |
| 23018 | SAVGWGVGQ |
| 23019 | SYIFSEIGT |
| 23020 | SYMVIGWGG |
| 23021 | TFTYAYDGQ |
| 23022 | TKKRNCFGM |
| 23023 | TSIHDNQGM |
| 23024 | VATSKDVGP |
| 23025 | VNRPTNFGT |
| 23026 | WPQRVPLGM |
| 23027 | YFPFHMAGQ |
| 23028 | EIRMIISGC |
| 23029 | GRHYIAAGY |
| 23030 | MLAFDPMGR |
| 23031 | HHMPIWLGL |
| 23032 | HFHEVAWGS |
| 23033 | AGQFKSEGS |
| 23034 | TGACMVEGL |
| 23035 | TLVSCDQGN |
| 23036 | DGGLRVQGM |
| 23037 | WKVSPIQGF |
| 23038 | EGGKNIFGM |
| 23039 | LDGPIGMGT |
| 23040 | EPLYSWVGI |
| 23041 | TNIKERTGS |
| 23042 | TIDVKRWGQ |
| 23043 | TSHLAWCEA |
| 23044 | VAMMGCMQA |
| 23045 | VCENEAGVA |
| 23046 | VMCQTLVKA |
| 23047 | VSLVRYVDA |
| 23048 | YPCFGTSLA |
| 23049 | AHTWLQHWA |
| 23050 | DAGWQQFAA |
| 23051 | DTEQWEATA |
| 23052 | HQYELQMHA |
| 23053 | LGYHCCQAA |
| 23054 | NSTPMACMA |
| 23055 | QIEYCMFYA |
| 23056 | SDDDREREA |
| 23057 | SHDLVAAWA |
| 23058 | THAWLQHWA |
| 23059 | SAQCAGHNA |
| 23060 | VLPEGTMTA |
| 23061 | LGMQADRFA |
| 23062 | LGYYCCQAA |
| 23063 | KYAYSLDCA |
| 23064 | GTEQWEATA |
| 23065 | WSKGTMLEA |
| 23066 | KGIWQGMDA |
| 23067 | NIRTGACSA |
| 23068 | KSEHHWINA |
| 23069 | FSEQNVSAA |
| 23070 | ASYYLVRNA |
| 23071 | DGERFMMQA |
| 23072 | DIGLRNMNA |
| 23073 | DTIQFVQNA |
| 23074 | DWKWKTMMA |
| 23075 | DYRGQDHVA |
| 23076 | EFTTCVELA |
| 23077 | EICFKEYWA |
| 23078 | EQIGVWWHA |
| 23079 | ETMWNMWDA |
| 23080 | IMITEVRSA |
| 23081 | KQETRGSVA |
| 23082 | KSCTTSHCA |
| 23083 | LSHQECAAA |
| 23084 | LTNDKSVTA |
| 23085 | MCRKNKILA |
| 23086 | MEGSDNLSA |
| 23087 | MHEGVAGIA |
| 23088 | MNEYMIKCA |
| 23089 | MYGFVMWEA |
| 23090 | NIYATGSQA |
| 23091 | NQVHSVDLA |
| 23092 | NYTPFYKRA |
| 23093 | PEYWGQKIA |
| 23094 | QFNFTESDA |
| 23095 | QTDWKVVVA |
| 23096 | SAHDTIRDA |
| 23097 | SFMVMACYA |
| 23098 | SICGTCQRA |
| 23099 | SSSCVVIAA |
| 23100 | SSTTVWEYA |
| 23101 | STGTNAEHA |
| 23102 | SYQWLCADA |
| 23103 | TSEGITREA |
| 23104 | TSSHQTVKA |
| 23105 | VCNMTDLQA |
| 23106 | VKESSDACA |
| 23107 | VMEQLQHTA |
| 23108 | WCTPMNRVA |
| 23109 | YKQSHMVAA |
| 23110 | MSWDMVEAA |
| 23111 | QFRCMMDDA |
| 23112 | EIVQPQCYA |
| 23113 | ALQFVSSTA |
| 23114 | TERRMFRTA |
| 23115 | QTPKSLLDA |
| 23116 | AEVMQWQAA |
| 23117 | EADTFTLIA |

### Example 12

AAV5 Variants with Tissue Tropism in Lung

This example describes engineered AAV5 variants with tissue tropism in lung that were discovered using the methods and systems described in EXAMPLE 1-EXAMPLE 2.

CNS, liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive lung tropism. The results are shown in FIG. 18E which shows the positional frequency of each amino acid in the 581 to 589 region of AAV5 VP1 in lung tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in lung over the frequency of that given amino acid residue occurring at the specified position in variants identified in all other harvested tissues was analyzed to identify a set of sequence rules for capsids that preferentially target lung tissue. With reference to TABLE 23 below, and SEQ ID NO: 2 (AAV5 VP1), the following amino acids can, thus, be independently mutated, in any combination, at any one or more positions Xaa1-Xaa9, to provide a VP1 capsid with enhanced lung tropism as compared wildtype AAV5 VP1 capsid (SEQ ID NO: 1), where lung tropism here refers to properties that are preferred for lung transduction over properties that are preferred for transduction of all other harvested tissues.

TABLE 23
Lung Tropism Rules

Xaa1 is selected from A, E, K, M, Q, R, S, or T
Xaa1 is selected from A, E, or Q
Xaa1 is E
Xaa2 is selected from A, I, K, S, T, or V
Xaa2 is selected from S, T, or V
Xaa2 is T
Xaa3 is selected from A, E, K, M, Q, R, S, T, or V
Xaa3 is selected from A, K, R, or S
Xaa3 is R
Xaa4 is selected from M, P, R, S, or T
Xaa4 is selected from P, Q, or T
Xaa4 is Q
Xaa5 is selected from I, K, L, M, T, V, or Y
Xaa5 is selected from L, M, or Y
Xaa5 is L
Xaa6 is selected from D, G, H, M, N, R, or S
Xaa6 is selected from H or N
Xaa6 is N
Xaa7 is selected from A, K, M, Q, or R
Xaa7 is selected from A, K or R
Xaa7 is R
Xaa8 is selected from A, F, G, S, W, or Y
Xaa8 is selected from A, F, or G
Xaa8 is F
Xaa9 is selected from A, E, G, P, R, or Y
Xaa9 is selected from G, P, or R
Xaa9 is G

TABLE 24 below provides sequences of the 581 to 589 region for variant AAV VP1 capsid polypeptides that were found in lung tissue and comport to one or more of the rules provided in TABLE 23. The present disclosure, thus, provides for rAAVs composed of engineered AAV5 VP1 capsid polypeptides having a SEQ ID NO: 2, wherein the Xaa1 to Xaa9 region is any one of SEQ ID NO: 16118-SEQ ID NO: 17117, as disclosed in TABLE 24. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 24
Sequences of the
581 to 589 Region
in AAV5 VP1 Capsid
Polypeptide that
Drive Lung Tissue
Tropism

| SEQ | |
|---|---|
| ID | 581-589 |
| NO | Sequence |
| 16118 | ETDYCYQCL |
| 16119 | EHVFGLLRE |
| 16120 | EDHISDSQL |
| 16121 | ENTEGIGPS |
| 16122 | EVTIHFVPQ |
| 16123 | EPRMSDMGD |
| 16124 | EPFCTQQLM |
| 16125 | EHPRQQDSA |
| 16126 | EVTRNHHFN |
| 16127 | ETRSGIAWQ |
| 16128 | EANLSHSWG |
| 16129 | EFLGTRLPD |
| 16130 | EGETVISQW |
| 16131 | EGRLKRHDF |
| 16132 | EGTEEGPTH |
| 16133 | ELRFVKTQR |
| 16134 | ENLARDAHY |
| 16135 | ERTIQHGEA |
| 16136 | ESTKAKKFP |
| 16137 | EVKLFKRNH |
| 16138 | EYKVYRHID |
| 16139 | EQKIKNGGH |
| 16140 | ERTCIATMW |
| 16141 | EVQRNIYSP |
| 16142 | EFSAMYSWY |
| 16143 | EHITEQDLR |
| 16144 | EACYAPSWA |
| 16145 | EAEYWNCGR |
| 16146 | EAGILMPAL |
| 16147 | EANSCEGFQ |
| 16148 | EANTWLNSA |
| 16149 | EARYSCWWG |
| 16150 | ECEQPNRWS |
| 16151 | ECFGKRFYD |
| 16152 | ECLPVLHND |
| 16153 | ECMGPGVCG |
| 16154 | ECQLCNWWP |
| 16155 | EDFWVFHYK |
| 16156 | EEEGQTAAG |
| 16157 | EEEQWGHMY |
| 16158 | EEVPCEKCF |
| 16159 | EEVRPKFGV |
| 16160 | EFCEMQNLC |
| 16161 | EFCHAMVCQ |
| 16162 | EFEDYGSWE |
| 16163 | EFEWECVQA |
| 16164 | EFFIIGAID |
| 16165 | EFKMSVFSN |
| 16166 | EFKQEFEAF |
| 16167 | EFTRKSCKF |
| 16168 | EGEVVDCDK |
| 16169 | EGGSQTYQG |
| 16170 | EGHQCQEGL |
| 16171 | EGRIHAQWM |
| 16172 | EGSPQESMS |
| 16173 | EGTGMQQAF |
| 16174 | EHADSWACP |
| 16175 | EHEGLNNHL |
| 16176 | EHFFKREDR |
| 16177 | EHFTKITVE |
| 16178 | EHFYICVHD |
| 16179 | EHNKEVYYG |
| 16180 | EHRQMHDSY |
| 16181 | EHSCTHMGN |
| 16182 | EHTRKDVCD |
| 16183 | EIHSNIAAG |
| 16184 | EILQGTVVQ |
| 16185 | EISMRPVTQ |
| 16186 | EIVYVDYPH |
| 16187 | EKCFCEVCG |
| 16188 | EKDHFDVMQ |
| 16189 | EKLNHEAGY |
| 16190 | EKMHQLATP |
| 16191 | EKRMMIDTH |
| 16192 | EKWMYDKDL |
| 16193 | ELDDICGIL |
| 16194 | ELEHYMSYQ |
| 16195 | ELKWWWRIF |
| 16196 | ELPTILKHR |
| 16197 | ELQKWFRKS |
| 16198 | EMEGYRAIQ |
| 16199 | EMFWQVAGA |
| 16200 | EMKFFAYAA |
| 16201 | EMKNLKRTG |
| 16202 | EMKVIGVDF |
| 16203 | EMRQQKMSR |
| 16204 | EMRTVMAGV |
| 16205 | EMTTSWYMN |
| 16206 | EMWSVKFGM |
| 16207 | ENAQFLVWA |
| 16208 | ENDSVSAIG |
| 16209 | ENGHCVSCT |
| 16210 | ENHRITGQS |
| 16211 | ENRKWTLAF |
| 16212 | ENSQTSAFG |
| 16213 | EPGELSVHV |
| 16214 | EPLTMDTGD |
| 16215 | EPRSCNFWC |
| 16216 | EQASSGERG |
| 16217 | EQMGYVHKG |
| 16218 | EQNTANLCD |
| 16219 | EQQFWNRLS |
| 16220 | EQQTGGYGP |
| 16221 | EQRFWNRLS |
| 16222 | EQVLMTYWF |
| 16223 | ERAEFEMKE |
| 16224 | ERAFSHNQS |
| 16225 | ERDHIKGCI |
| 16226 | ERSDFHWYM |
| 16227 | ESDWMNVSM |
| 16228 | ESFNYAHFK |
| 16229 | ESHPFTNFS |
| 16230 | ESISRDQSW |
| 16231 | ESKMSHKEA |
| 16232 | ESLHALAHM |
| 16233 | ESMFWDSDK |
| 16234 | ESMRRLGDT |
| 16235 | ESNSNRWAY |
| 16236 | ESRIMAPLW |
| 16237 | ESWNILLTH |
| 16238 | ETADRPSLD |
| 16239 | ETAHCPLNA |
| 16240 | ETCIANAWL |
| 16241 | ETDDQMMPP |
| 16242 | ETHTSEANM |
| 16243 | ETKLLPDLG |
| 16244 | ETMAQSCYG |
| 16245 | ETMGICHNS |
| 16246 | ETMHPVVDG |
| 16247 | ETMSTVYGQ |
| 16248 | ETNSCEGFQ |
| 16249 | ETRQAHLYH |
| 16250 | ETTAQKLKI |
| 16251 | ETVEYLSYE |
| 16252 | EVASKFFEH |
| 16253 | EVCRIGWMS |
| 16254 | EVCTIMEHP |
| 16255 | EVGTSCRFE |
| 16256 | EVNDQYREL |
| 16257 | EVNMPTGQH |
| 16258 | EVPHFDVSQ |
| 16259 | EVQCCMAAM |
| 16260 | EVQPVRRFT |
| 16261 | EVSFKARCA |
| 16262 | EVSMPEKTP |
| 16263 | EVSMTGHIA |
| 16264 | EVSPMRHIM |
| 16265 | EVTNEHCCW |
| 16266 | EVTQFDNPV |
| 16267 | EVTQGECFY |
| 16268 | EVVHDPGYA |
| 16269 | EVVKCPNSI |
| 16270 | EVVKIDMIP |
| 16271 | EVYAPRHMC |
| 16272 | EWDKMQKVP |
| 16273 | EWGRRANKD |
| 16274 | EWHAFMSHC |
| 16275 | EWWRYMADY |
| 16276 | EYCWANKNG |
| 16277 | EYIREWAQD |
| 16278 | EYKQQGVRR |
| 16279 | EYQGNYMAT |
| 16280 | EMMERQIHE |
| 16281 | EFITSGQCS |
| 16282 | EVVFMSGDI |
| 16283 | EPGHPGYEM |
| 16284 | EVMGLGCDG |
| 16285 | EAQWGMDDT |
| 16286 | ECKTHRDVY |
| 16287 | ENIPQSIWT |
| 16288 | ETCWTACDR |
| 16289 | EAYTDMPKT |
| 16290 | NTRPMRLHM |
| 16291 | NTRPMRPHM |
| 16292 | QTMQWMCKG |
| 16293 | QTMWLKEHR |
| 16294 | RTEIRYYGT |
| 16295 | STRPAIQKS |
| 16296 | STTVWAQCS |
| 16297 | STVMESAMY |
| 16298 | TTGTFPALP |
| 16299 | TTMPKGGNL |
| 16300 | VTESWSSWQ |
| 16301 | WTCLCYKFM |
| 16302 | YTEQEDNAA |
| 16303 | KTYCQDGWT |
| 16304 | LTTVYNWPH |
| 16305 | ITPPYCDMV |
| 16306 | LTMMAMCND |
| 16307 | ATQTDPVAR |
| 16308 | HTLHSHQNT |
| 16309 | STERGTPQY |
| 16310 | TTGTFPALQ |
| 16311 | ATVMFIEIS |
| 16312 | FTNQLPEAN |
| 16313 | CTLRVEYVQ |
| 16314 | VTWMVKTSN |
| 16315 | ATQFSGENA |
| 16316 | ATQLYAPIA |
| 16317 | FTVYVGARE |
| 16318 | KTKCCKTHD |
| 16319 | KTQIHMGDE |
| 16320 | RTEAHLPAP |
| 16321 | TTERYEITP |
| 16322 | VTVYVGARE |
| 16323 | ITASIKDQW |
| 16324 | DTAPNESDR |
| 16325 | HTRKEGENS |
| 16326 | GTWMEENLH |
| 16327 | RTDVMMKVM |
| 16328 | ATEMFDCDV |
| 16329 | ATNVLYLDC |
| 16330 | ATQQVFWNL |
| 16331 | ATSHYAKGL |
| 16332 | ATSIFCVFR |
| 16333 | ATTHWAGHH |
| 16334 | ATTMSAFYG |
| 16335 | ATTSYNLTR |
| 16336 | ATVDISNVK |
| 16337 | CTPPIVMDR |
| 16338 | CTSFACKYF |
| 16339 | DTAMMHKIA |
| 16340 | DTGEIMTRS |
| 16341 | DTIPYCRGC |
| 16342 | DTKFWMQWN |
| 16343 | DTKIQTIHT |
| 16344 | DTLLPANWS |
| 16345 | DTNKLNDLS |
| 16346 | DTRQCLDTQ |
| 16347 | DTTTSTMAL |
| 16348 | DTVNTCGQM |
| 16349 | FTELHCNGN |
| 16350 | GTEYDRLSP |
| 16351 | GTFMDQRPN |
| 16352 | GTMPMDAGG |
| 16353 | GTMTTLYAN |
| 16354 | GTPHVVSQE |
| 16355 | GTQVNRATE |
| 16356 | GTSVNMKDQ |
| 16357 | GTSYWPQWN |
| 16358 | HTAICQDAS |
| 16359 | HTAYPYQCP |
| 16360 | HTDCLFTDD |
| 16361 | HTIPFFEAE |
| 16362 | HTKMTHGTS |
| 16363 | HTMQREYME |
| 16364 | HTNCLFTDD |
| 16365 | HTTTQVKHP |
| 16366 | HTVKIDQTC |
| 16367 | ITDQRRRAC |
| 16368 | ITELRPLIL |
| 16369 | ITNKQQLAR |
| 16370 | ITNQFMSKH |
| 16371 | ITRVDCSHD |
| 16372 | KTEPSVRIA |
| 16373 | KTLMWDCQT |
| 16374 | KTTILEDKA |
| 16375 | KTYICASCF |
| 16376 | LTAVSVFRR |
| 16377 | LTHDGWFYS |
| 16378 | LTKCNEKGN |
| 16379 | LTMLHMHCQ |
| 16380 | LTSGEGFEV |
| 16381 | LTTGIWKTW |
| 16382 | LTWMGLSME |
| 16383 | LTYVQGDWR |
| 16384 | MTEQSGRAY |
| 16385 | MTETTDMAH |
| 16386 | MTFFVNQAH |
| 16387 | MTIGFHVNG |
| 16388 | MTKQNKTHR |
| 16389 | MTTSSHWAK |
| 16390 | MTVQWAQYG |
| 16391 | NTAPKMSCT |
| 16392 | NTDVPQFMP |
| 16393 | NTEPMWSCS |
| 16394 | NTEVMDTGQ |
| 16395 | NTLRPQHTF |
| 16396 | NTMEDWECY |
| 16397 | NTNFWNWSL |
| 16398 | NTRSKDFWN |
| 16399 | NTSAHGYNQ |
| 16400 | NTTTFCHDE |
| 16401 | NTVFMHEQT |
| 16402 | NTVSHFAYD |
| 16403 | QTAANFCFD |
| 16404 | QTDAQGIGH |
| 16405 | QTELDTGIS |
| 16406 | QTFLAMDGK |
| 16407 | QTHDEYECA |
| 16408 | QTIRDMSGF |
| 16409 | QTLLSNCCA |
| 16410 | QTLWVHGTR |
| 16411 | QTMPCMYVE |
| 16412 | QTNVELHQE |
| 16413 | QTPQYHVGG |
| 16414 | QTQLNCAAC |
| 16415 | QTSFAETDY |
| 16416 | QTTVFQLEN |
| 16417 | QTVQLGTPV |
| 16418 | QTWAAHCSR |
| 16419 | QTYHVYTCC |
| 16420 | RTDRNTDFL |
| 16421 | RTGFEVPTS |
| 16422 | RTHLMEVTQ |
| 16423 | STDLKFCAK |
| 16424 | STDNKFWLG |
| 16425 | STEAAYENP |
| 16426 | STERMFKEA |
| 16427 | STFTAGATM |
| 16428 | STHGHISHN |
| 16429 | STMNFLPCE |
| 16430 | STMSCEILR |
| 16431 | STMTCGEFA |
| 16432 | STSDHHCHS |
| 16433 | STSSLDKLI |
| 16434 | STTNPYQLA |
| 16435 | TTAMYPVHT |
| 16436 | TTDCMWENH |
| 16437 | TTDQKRHGS |
| 16438 | TTEQWDFCR |
| 16439 | TTERTRICN |
| 16440 | TTMPHHEWQ |
| 16441 | TTMQCNSYD |
| 16442 | TTNPVEMFN |
| 16443 | TTTHMLTRC |
| 16444 | TTVPFQAWC |
| 16445 | TTYCPPTVE |
| 16446 | VTDLVRHFA |
| 16447 | VTEHQQRSV |
| 16448 | VTENLMMYH |
| 16449 | VTEQDDGQW |
| 16450 | VTKEMVWQC |
| 16451 | VTNQCCKAM |
| 16452 | VTTQSCKWM |
| 16453 | WTDNYFHES |
| 16454 | WTLHNQSLK |
| 16455 | WTVHNKDEA |
| 16456 | YTKAEDSIC |
| 16457 | YTKQIDNIE |
| 16458 | STMMVGRQC |
| 16459 | ITNSERATY |
| 16460 | PTDVITTDQ |
| 16461 | DTITFKQQH |
| 16462 | MTTEHITWW |
| 16463 | ITAIMCRSS |
| 16464 | CTESCQTGP |
| 16465 | MTESVENPG |
| 16466 | STTAYHQDY |
| 16467 | CTEIRYYGT |
| 16468 | QERGSLWYG |
| 16469 | WWRAMEKDM |
| 16470 | LDRNQSYHA |
| 16471 | SLRTPECDC |
| 16472 | CYRCALWTV |
| 16473 | ANRFYDCSH |
| 16474 | DRREKQLMK |
| 16475 | DVRAMHSTC |
| 16476 | FCRMYANAD |
| 16477 | IGRLNASTP |
| 16478 | IRRSMQQQN |
| 16479 | PSRWYKLGI |
| 16480 | TIRYIGMSG |
| 16481 | ASRSCMTQE |
| 16482 | AERQACTMG |
| 16483 | VGRERVWDS |
| 16484 | ALRPYMDDR |
| 16485 | ASRYDYNHG |
| 16486 | ASRYDYSHG |
| 16487 | CERQALKMI |
| 16488 | CLRQDRIIF |
| 16489 | DARPSPISQ |
| 16490 | DARVSNIQQ |
| 16491 | DCRCYGNNS |
| 16492 | DKRDHHYFA |
| 16493 | DSRFYEVQL |
| 16494 | DSRQLHHIG |
| 16495 | DSRYTQCYI |
| 16496 | DVRMNQDLY |
| 16497 | FGRNYTDHL |
| 16498 | FGRYRLWKG |
| 16499 | GERWPHNML |
| 16500 | GFRVGSLRH |
| 16501 | GIRLKGNIM |
| 16502 | GSRPRMKQP |
| 16503 | HCRLKEDEG |
| 16504 | HERTAKFRY |
| 16505 | HRRLFNKMN |
| 16506 | IERNSGQGS |
| 16507 | IGRDIDTSF |
| 16508 | INRENRRGG |
| 16509 | ISRMCESAL |
| 16510 | KLREVGRPC |
| 16511 | KQRDYMRKR |
| 16512 | LMRWPWDDT |
| 16513 | LNRILDRMG |
| 16514 | MGRTCADWT |
| 16515 | MIRNETQWA |
| 16516 | MVRFAPAYP |
| 16517 | NMRYTEMGV |
| 16518 | QCRNQDKSQ |
| 16519 | QERWQIQTW |
| 16520 | QSRLEHYGL |
| 16521 | SHREMNRAR |
| 16522 | SPREKNHYW |
| 16523 | TAREDCQHA |
| 16524 | TARLIEKYN |
| 16525 | TVRWQADTM |
| 16526 | VERHKMKWQ |
| 16527 | VSRSLERQA |
| 16528 | WCRQTMMDW |
| 16529 | WFRRHNYFY |
| 16530 | WNRDHNDWT |
| 16531 | DVRSHDEMT |
| 16532 | TERRMFRTA |
| 16533 | QVRFEQNPA |
| 16534 | MDRMHKSGI |
| 16535 | KNRDFEGFD |
| 16536 | NQDQARTEH |
| 16537 | QAFQFECHD |
| 16538 | QATQAGANQ |
| 16539 | RMAQFFRAH |
| 16540 | RNLQGGNHG |
| 16541 | SCLQYKATE |
| 16542 | SIEQKLTFT |
| 16543 | VGWQYCEEG |
| 16544 | TRKQGEGPY |
| 16545 | TGHQLPYNK |
| 16546 | LIDQNTKGN |
| 16547 | CGYQFSETM |
| 16548 | SGCQYEPRN |
| 16549 | IQAQMDQQQ |
| 16550 | CFNQRIQDS |
| 16551 | MGLQNDPTS |
| 16552 | TATQREAFV |
| 16553 | IAEQRLVEV |
| 16554 | QPSQQGRPG |
| 16555 | AVDQLRMFL |
| 16556 | CDHQRHQQP |
| 16557 | FRKQGHGEK |
| 16558 | IIGQWGQSD |
| 16559 | IIGQWGRSD |
| 16560 | NELQQGTSE |
| 16561 | NVSQWMITY |
| 16562 | QYVQCAAKD |
| 16563 | SLTQYQWRG |
| 16564 | VDMQKQTES |
| 16565 | VNVQMPSAP |
| 16566 | WAMQWWAKP |
| 16567 | YESQKGRRA |
| 16568 | MGMQQDTQV |
| 16569 | AAQQGLNPC |
| 16570 | ADIQISLCD |
| 16571 | AFAQYSRGN |
| 16572 | AFNQQEMWA |
| 16573 | ANHQVTSHP |
| 16574 | ANMQPNAIM |
| 16575 | ASGQFPKEG |
| 16576 | ASLQATCNH |
| 16577 | AYAQKYRTA |
| 16578 | CFSQTDCMS |
| 16579 | CIWQHNITQ |
| 16580 | CSQQVNCLS |
| 16581 | CWMQSQENW |
| 16582 | DCAQITYGG |
| 16583 | DCAQLRQDG |
| 16584 | DFVQSDTEF |
| 16585 | DGLQIQIQE |
| 16586 | DKMQCSSGG |
| 16587 | DMAQQQKSH |
| 16588 | DVMQGNRAF |
| 16589 | DWAQFCNHT |
| 16590 | DYAQFSVVP |
| 16591 | DYAQGAAMP |
| 16592 | FGMQRRHKE |
| 16593 | FHKQFHSKF |
| 16594 | GDEQTHCNG |
| 16595 | GFTQFYHHE |
| 16596 | GIHQETYCP |
| 16597 | GITQMSQHA |
| 16598 | GMVQDYVWL |
| 16599 | GSMQVMKKC |
| 16600 | GVCQMSEMR |
| 16601 | GVFQEASHK |
| 16602 | GVTQGHEIT |
| 16603 | GWMQWHRQR |
| 16604 | GYEQPVKCE |
| 16605 | GYIQCQMHA |
| 16606 | HASQEYTSL |
| 16607 | HASQLGAMV |
| 16608 | HDAQFLPDQ |
| 16609 | HGLQVGQFY |
| 16610 | HPEQFVACE |
| 16611 | HSNQMWYCY |
| 16612 | HWAQLKVTY |
| 16613 | IHKQFEMES |
| 16614 | IHQQTHYQF |
| 16615 | IMMQRALMG |
| 16616 | INQQYNSCW |
| 16617 | ISTQMENTG |
| 16618 | IVEQKPTVQ |
| 16619 | KDAQQWNCA |
| 16620 | KGNQQITSL |
| 16621 | KMIQSTHDF |
| 16622 | KPNQSNRQE |
| 16623 | KSAQVYWNP |
| 16624 | LAVQSIHSH |
| 16625 | LHCQIWFQD |
| 16626 | LIAQQMHIN |
| 16627 | LICQSDCFP |
| 16628 | LMTQEAQRA |
| 16629 | LSVQAQWDV |
| 16630 | LWMQHMCLD |
| 16631 | LYCQSPKDC |
| 16632 | MAHQSDIMH |
| 16633 | MAQQYHDEM |
| 16634 | MGHQWSGNK |
| 16635 | NFEQYVHNY |
| 16636 | NFKQPNHWD |
| 16637 | NHQQQNLIG |
| 16638 | QAEQENFDH |
| 16639 | QMAQREDDY |
| 16640 | QPAQMEDMG |
| 16641 | QSAQQPAGS |
| 16642 | QVFQLTMVK |
| 16643 | QVGQYTLPE |
| 16644 | RCWQADISG |
| 16645 | RPAQIDPWY |
| 16646 | SAVQIQVLH |
| 16647 | SLAQDIACF |
| 16648 | SNDQNIHNG |
| 16649 | SNEQIPRVP |
| 16650 | SQSQFCAQV |
| 16651 | SQTQWDHMD |
| 16652 | SSEQAGPHM |
| 16653 | SVQQIQKQP |
| 16654 | TFHQCERSD |
| 16655 | THQQVALGN |
| 16656 | THTQYWKYA |
| 16657 | TISQMVYSK |
| 16658 | TMGQICVHA |
| 16659 | TQCQWNVAK |
| 16660 | VEAQFMDNA |
| 16661 | VLSQYTQHA |
| 16662 | VVSQTYQNR |
| 16663 | VWQQYILVI |
| 16664 | WYIQVDRKG |
| 16665 | YIKQIDNIE |
| 16666 | YQTQYSFWH |
| 16667 | LVGQCTSHW |
| 16668 | GIKQTLVRQ |
| 16669 | GCQQIQLQT |
| 16670 | KYMQENVLS |
| 16671 | NCHQLGDNM |
| 16672 | MMVQWGAMA |
| 16673 | FSEQNVSAA |
| 16674 | TSGQVDHRT |
| 16675 | YYEQMDGEF |
| 16676 | QPVQAPHCA |
| 16677 | SIAQDQVWS |
| 16678 | PLKALIWEY |
| 16679 | QMEALMDCE |
| 16680 | QSKTLCYQC |
| 16681 | QVSTLENVQ |
| 16682 | RQAKLATLH |
| 16683 | SKESLPQWQ |
| 16684 | TGVSLVNYS |
| 16685 | MLHNLWMQC |
| 16686 | AAQTLECHK |
| 16687 | YWFVLNPCV |
| 16688 | CHVNLKAWW |
| 16689 | DFGMLEWQW |
| 16690 | KYEILNCNH |
| 16691 | MQYTLPTWP |
| 16692 | TMANLCWRT |
| 16693 | TNIPLGTNM |
| 16694 | TWMPLAPCH |
| 16695 | TWMPLAPCY |
| 16696 | HQNLLICNE |
| 16697 | TGKWLVYQA |
| 16698 | IPADLDVDH |
| 16699 | FFLKLNDVP |
| 16700 | AHYFLLEYP |
| 16701 | AIIPLPMPT |
| 16702 | AIVNLHTQQ |
| 16703 | ANLPLQQQD |
| 16704 | AVEFLAHRY |
| 16705 | AVYPLFAPD |
| 16706 | CHNLLHCVY |
| 16707 | CNSTLQYGH |
| 16708 | DFCMLQMGM |
| 16709 | DHCILPQTC |
| 16710 | DMFMLDEAP |
| 16711 | DYFPLVVAP |
| 16712 | GANFLVSQT |
| 16713 | GKNPLMPSP |
| 16714 | GLDKLMMLA |
| 16715 | GVMMLGTAN |
| 16716 | HIDHLNAAT |
| 16717 | HPLPLSVMS |
| 16718 | HSMLLQEYM |
| 16719 | INTALFVNA |
| 16720 | KEYDLSLTM |
| 16721 | KNFELDQFE |
| 16722 | KPYSLAMTI |
| 16723 | LGTPLEAGL |
| 16724 | LIDRLPACY |
| 16725 | LRDCLHVLG |
| 16726 | LVDELNQIK |
| 16727 | MFDDLMVGR |
| 16728 | MINNLDECC |
| 16729 | MVDGLTKEY |
| 16730 | NCQSLATNV |
| 16731 | NEVHLEHNY |
| 16732 | NLEMLAMFQ |
| 16733 | NVMPLIQYR |
| 16734 | PITILCCSQ |
| 16735 | PRQLLHFSQ |
| 16736 | QASLLDHYP |
| 16737 | QGQPLMRYS |
| 16738 | QQNRLNMNW |
| 16739 | QQSPLLWER |
| 16740 | QRHDLEVDH |
| 16741 | QVVDLVDVY |
| 16742 | RGEILQLQQ |
| 16743 | SAQCLEMWP |
| 16744 | SGLILQCNP |
| 16745 | SINYLGEGH |
| 16746 | SSKPLTNEC |
| 16747 | SVAFLNQEW |
| 16748 | SWNRLNWCL |
| 16749 | TCESLYSRG |
| 16750 | THDSLIAHW |
| 16751 | TIGNLTKPI |
| 16752 | TLMGLWNQG |
| 16753 | TNDPLMRSD |
| 16754 | TQCGLKMYP |
| 16755 | TRMLLEWTY |
| 16756 | TSEPLEGID |
| 16757 | VDSWLSMRD |
| 16758 | VIHYLTHEC |
| 16759 | YCIHLKDDI |
| 16760 | YCIPLQPMN |
| 16761 | YSVPLHHCF |
| 16762 | YVGNLCRWK |
| 16763 | YVVKLEDYK |
| 16764 | YYADLNAHD |
| 16765 | RNMALHHPT |
| 16766 | DGCSLYAFN |
| 16767 | ACDRLMFQG |
| 16768 | SLVDLYKTA |
| 16769 | FCNKLTSVR |
| 16770 | IDAFLANRG |
| 16771 | NMMTLWSHL |
| 16772 | QIVSKNSRQ |
| 16773 | RGVHSNCAE |
| 16774 | SAFLPNTVC |
| 16775 | SEYDNNPCQ |
| 16776 | SFTIYNHKW |
| 16777 | SSMTRNNCK |
| 16778 | THDYFNGDY |
| 16779 | TWANGNGGP |
| 16780 | VNHHENWKK |
| 16781 | IEWPTNSDH |
| 16782 | NAYVDNLRY |
| 16783 | QQCMGNDMT |
| 16784 | AFLHENMWG |
| 16785 | ANGTYNLQE |
| 16786 | ANMFRNGMQ |
| 16787 | DSYFINELY |
| 16788 | FFEMVNTEQ |
| 16789 | HSWINNQEH |
| 16790 | KSVTQNRTR |
| 16791 | NSYFINELY |
| 16792 | TFAAVNCCQ |
| 16793 | TMKTFNYVF |
| 16794 | YCQLGNHNA |
| 16795 | YHENWNMPM |
| 16796 | GWMRHNRIQ |
| 16797 | QSGYMNKLW |
| 16798 | VSMSSNYDK |
| 16799 | KCAMFNAGE |
| 16800 | AEYMTNASM |
| 16801 | AGYASNWAD |
| 16802 | AISPTNAER |
| 16803 | AKGTYNQGN |
| 16804 | ASETNNGGY |
| 16805 | AYQMFNNVQ |
| 16806 | DAEHKNAIR |
| 16807 | DAHYTNECR |
| 16808 | DCQPVNRIL |
| 16809 | DINFDNHAG |
| 16810 | DMNSRNVQD |
| 16811 | DMVPCNLMH |
| 16812 | DWAANNWWP |
| 16813 | FEAPINLSF |
| 16814 | FVHVINRCD |
| 16815 | GAMPGNGCT |
| 16816 | GHKVCNCWE |
| 16817 | GVWPDNCPQ |
| 16818 | GYCGCNHMG |
| 16819 | HHVWPNRQV |
| 16820 | HIEMRNAHM |
| 16821 | HMYVTNHDG |
| 16822 | ICSYYNQTQ |
| 16823 | IFYATNHSW |
| 16824 | IKHDFNSAF |
| 16825 | INAPPNLRT |
| 16826 | KGDEQNCYC |
| 16827 | KIDVENCCC |
| 16828 | KPGTVNPAA |
| 16829 | KQEPCNSGH |
| 16830 | LCAGTNLTP |
| 16831 | LEADNNDAD |
| 16832 | LKIPFNMTC |
| 16833 | LMAYANTDD |
| 16834 | LRTPYNFWW |
| 16835 | LVKVINERQ |
| 16836 | MCTPRNEQE |
| 16837 | MKGFDNYSP |
| 16838 | MLSYHNESW |
| 16839 | MQNEKNDNP |
| 16840 | MRKDYNDTW |
| 16841 | MYQDANQIT |
| 16842 | NVHTFNYAF |
| 16843 | QFSGYNYHY |
| 16844 | QLHWPNNFG |
| 16845 | QMHVANINL |
| 16846 | QSNEFNPVA |
| 16847 | QVAPKNCWQ |
| 16848 | QYICFNCYH |
| 16849 | SACEPNVSP |
| 16850 | SANKMNAER |
| 16851 | SFGTCNTCG |
| 16852 | SIAMYNNMH |
| 16853 | SREAQNRET |
| 16854 | SVKDNNRRL |
| 16855 | SWTASNWRD |
| 16856 | SWTSRNAHH |
| 16857 | TAQYVNREI |
| 16858 | TQDVQNMYV |
| 16859 | TSMLHNEEN |
| 16860 | TVKEENAAC |
| 16861 | VFIAQNEGN |
| 16862 | VGVGVNRFD |
| 16863 | VHANANMSH |
| 16864 | VPELGNGAY |
| 16865 | VQLPTNKQW |
| 16866 | VSINTNEPG |
| 16867 | VVQYKNHAT |
| 16868 | YGTEVNSMI |
| 16869 | YQVANNQQG |
| 16870 | YYADMNAHD |
| 16871 | HMWMYNKCT |
| 16872 | ARTIANDTQ |
| 16873 | FREDMNATM |
| 16874 | SLWVGYRTD |
| 16875 | TPKNDQRAE |
| 16876 | LNARTMRSA |
| 16877 | MEDFSDRWP |
| 16878 | RMVIMERDR |
| 16879 | DGQLVERYD |
| 16880 | APKNDQRAE |
| 16881 | TYYWDCRHV |
| 16882 | LMEAASRSY |
| 16883 | TQQTADRWA |
| 16884 | IVTCNGRWD |
| 16885 | YDYYKMREG |
| 16886 | ASKAKQRVA |
| 16887 | FQATTKRLP |
| 16888 | NAQLGKRGH |
| 16889 | YLWMIARPD |
| 16890 | YLWMITRPD |
| 16891 | KQNPSMRQD |
| 16892 | SATARFRDN |
| 16893 | AAEMHQRGE |
| 16894 | AAEMHQRGV |
| 16895 | AFKATRRGV |
| 16896 | ASGSHQREN |
| 16897 | ASGSHQRKN |
| 16898 | ASHIYCRHE |
| 16899 | DIKMQKRGV |
| 16900 | DVGLALRNH |
| 16901 | FQADYKRHE |
| 16902 | FVKMKQRYG |
| 16903 | GAGYRMRVN |
| 16904 | GVSVEGRDE |
| 16905 | GYKNCHRHG |
| 16906 | HIDLQTRKE |
| 16907 | HVEWTMREH |
| 16908 | IGGMFSRHA |
| 16909 | IQKLMRRMH |
| 16910 | KAISHERGQ |
| 16911 | LAYTCHRGD |
| 16912 | LHWYWCRNW |
| 16913 | LKKKRGRAC |
| 16914 | LQEPRMRYE |
| 16915 | MCQSYERGA |
| 16916 | MFNRIPRFS |
| 16917 | MICRMDRMK |
| 16918 | MQKAHDRSA |
| 16919 | MSGFHIRCA |
| 16920 | MSSFHIRCA |
| 16921 | NLIYQPRKS |
| 16922 | QATFTTRLV |
| 16923 | QGDHFDRNS |
| 16924 | QGGMMKRGQ |
| 16925 | QIDEVTRNL |
| 16926 | QVEPVEREY |
| 16927 | RCDGVMRSQ |
| 16928 | SAELVQREA |
| 16929 | SGSHCGRMA |
| 16930 | SLKMRPRQI |
| 16931 | SRGDYLRVG |
| 16932 | SWEWQMRYP |
| 16933 | TAIHEHRGL |
| 16934 | TIEPFWRSW |
| 16935 | TKYVDDRPG |
| 16936 | TSIPITRQE |
| 16937 | VINMNRRWG |
| 16938 | VSINMWRAQ |
| 16939 | WDNLMQRTC |
| 16940 | WIQPISRTG |
| 16941 | YGSYYMRGW |
| 16942 | YMHEWMRLT |
| 16943 | YSAPRIRDA |
| 16944 | SMNLWCRGN |
| 16945 | CDLFPIRWP |
| 16946 | TEGPMHRAW |
| 16947 | QGTGHSLFL |
| 16948 | RHWRHTMFL |
| 16949 | RMETWCGFS |
| 16950 | PMWTQMPFP |
| 16951 | DINASEWFC |
| 16952 | MIVMGPHFT |
| 16953 | SLYNCMFFL |
| 16954 | WMKVRQGFG |
| 16955 | ILEAIVNFW |
| 16956 | KISPWDGFY |
| 16957 | AALENGWFN |
| 16958 | ACMVNPGFW |
| 16959 | ACSTVMDFN |
| 16960 | ASIWRKLFD |
| 16961 | AVHAEDIFM |
| 16962 | DASVPGVFE |
| 16963 | DCNWHWPFT |
| 16964 | DKHFYQKFH |
| 16965 | DSSLHGAFM |
| 16966 | DSVNTSAFH |
| 16967 | DVIRDDSFQ |
| 16968 | FGMVHHGFF |
| 16969 | FKYYIPEFC |
| 16970 | FPGWCASFP |
| 16971 | HICDMDMFL |
| 16972 | HYFYTQQFG |
| 16973 | IAIPCASFY |
| 16974 | KNVMHSEFR |
| 16975 | KYDREWPFL |
| 16976 | LCCDVCMFL |
| 16977 | LGDTGLMFG |
| 16978 | LMSICAAFP |
| 16979 | LQGWRWWFA |
| 16980 | LVSDHIEFG |
| 16981 | MASSEHCFF |
| 16982 | MHIEHVMFQ |
| 16983 | MIWYNRKFM |
| 16984 | MNEIFVEFP |
| 16985 | MWTAQQTFM |
| 16986 | MYIPVYTFN |
| 16987 | NVQPESTFF |
| 16988 | PAMPFYWFE |
| 16989 | QHQPFSMFS |
| 16990 | QNSHGPVFG |
| 16991 | QNVMHSEFR |
| 16992 | QQAPATIFP |
| 16993 | QQVEWSVFQ |
| 16994 | QSISDMPFP |
| 16995 | QVTPSMIFK |
| 16996 | RDSCYINFP |
| 16997 | SATCWLWFG |
| 16998 | SCMGFTSFP |
| 16999 | SDEWEDVFG |
| 17000 | SGCHWGIFH |
| 17001 | SMYSTHCFC |
| 17002 | SYPGHHWFY |
| 17003 | TASGNTYFV |
| 17004 | TILEGDNFA |
| 17005 | TITFIDLEN |
| 17006 | TKNAMHNFF |
| 17007 | TVSRASYFD |
| 17008 | VAVTVTDFD |
| 17009 | VMEKYAHFP |
| 17010 | WGDRYGLFV |
| 17011 | YSQWFTFFE |
| 17012 | QVHAKLGFM |
| 17013 | SATIRFYFN |
| 17014 | QHQAFFVFT |
| 17015 | QMKGATEFC |
| 17016 | LVNMFQCFW |
| 17017 | ASTERAKFG |
| 17018 | NVKWHCDGG |
| 17019 | PYFRFFPHG |
| 17020 | QCGAIYQTG |
| 17021 | QQNLADQAG |
| 17022 | RQGKAMSMG |
| 17023 | SCTHIKVPG |
| 17024 | SCTMKDWWG |
| 17025 | SCTPIKVPG |
| 17026 | FLNNEELKG |
| 17027 | WDNMDQTQG |
| 17028 | AMLRQEKTG |
| 17029 | DSQNNWPTG |
| 17030 | KCGWHGEHG |
| 17031 | VQQNNLKAG |
| 17032 | VAANTYCYG |
| 17033 | GEPWFQTHG |
| 17034 | GEPWFRTHG |
| 17035 | GMASMEHCG |
| 17036 | GQGTSRLMG |
| 17037 | ICHLRHIAG |
| 17038 | MMLGYMEQG |
| 17039 | NAKPKQVQG |
| 17040 | PCYNAMNRG |
| 17041 | QIHPYLEAG |
| 17042 | SRDCVVHDG |
| 17043 | VCPSCRDYG |
| 17044 | WIKWKCSEG |
| 17045 | CLNNSTCRG |
| 17046 | QHSKCHMHG |
| 17047 | AADFSGYYG |
| 17048 | AAGATMLRG |
| 17049 | AEIPIMFMG |
| 17050 | AFVNWPIHG |
| 17051 | ANQPKHGAG |
| 17052 | APLIKASWG |
| 17053 | AWDWCDMRG |
| 17054 | AYNSRANGG |
| 17055 | CHGHFQHIG |
| 17056 | DAKTCHSRG |
| 17057 | DCKLMRHQG |
| 17058 | DIMLRTCCG |
| 17059 | DRCMQTKHG |
| 17060 | DSATQHMEG |
| 17061 | DWTTISHEG |
| 17062 | FEGSRQLWG |
| 17063 | FREWRAMDG |
| 17064 | GCTPSVCSG |
| 17065 | GVMLKMAGG |
| 17066 | GYVPWLMEG |
| 17067 | HCHMIFKDG |
| 17068 | HKDDYVQTG |
| 17069 | ISEDIKENG |
| 17070 | ISWLCEMNG |
| 17071 | KNEWTYHQG |
| 17072 | LEDMMSVEG |
| 17073 | LIMEQPLAG |
| 17074 | LNAECTSTG |
| 17075 | LSTCWAIAG |
| 17076 | LVVNQYTLG |
| 17077 | MAAPTALNG |
| 17078 | MIGTTQSDG |
| 17079 | MKASTMELG |
| 17080 | MKVYHEMVG |
| 17081 | MQVSADHHG |
| 17082 | NAAWQSITG |
| 17083 | NCSHYDSRG |
| 17084 | NGMVSGQYG |
| 17085 | NHPYQHIEG |
| 17086 | NKMHCDTMG |
| 17087 | NKVMCLWQG |
| 17088 | NNAPVWLHG |
| 17089 | NSVSCGGQG |
| 17090 | PGDFTSGYG |
| 17091 | QCDEQQEAG |
| 17092 | QFGSFWPTG |
| 17093 | QNARSSMVG |
| 17094 | QYDTIQLNG |
| 17095 | SCHHVGMCG |
| 17096 | SFKWTIVNG |
| 17097 | SIMKYDQMG |
| 17098 | SMHHTQFTG |
| 17099 | SVGFPYESG |
| 17100 | TAQYDVWHG |
| 17101 | TGYPKLKDG |
| 17102 | TQEPWGCAG |
| 17103 | TQGLPRGRG |
| 17104 | TRLYNWPMG |
| 17105 | TSHHWAAQG |
| 17106 | TWWKDPQEG |
| 17107 | VAHYYCWEG |
| 17108 | VLMNWQPRG |
| 17109 | VLNHWEPIG |
| 17110 | VVMHPYAAG |
| 17111 | VVSHYDMIG |
| 17112 | WHTVHYMGG |
| 17113 | WLTFDRFEG |
| 17114 | AKCPWHHTG |
| 17115 | VNPWYVKHG |
| 17116 | DVASRCWVG |
| 17117 | SGTPHQQSG |

### Example 13

AAV5 Variants with Tissue Tropism in Heart

This example describes engineered AAV5 variants with tissue tropism in heart that were discovered using the methods and systems described in EXAMPLE 1-EXAMPLE 2.

CNS, liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive heart tropism. The results are shown in FIG. 18D which shows the positional frequency of each amino acid in the 581 to 589 region of AAV5 VP1 in heart tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in heart over the frequency of that given amino acid residue occurring at the specified position in variants identified in all other harvested tissues was analyzed to identify a set of sequence rules for capsids that preferentially target heart tissue. With reference to TABLE 25 below, and SEQ ID NO: 2 (AAV5 VP1), the following amino acids can, thus, be independently mutated, in any combination, at any one or more positions Xaa1-Xaa9, to provide a VP1 capsid with enhanced heart tropism as compared wildtype AAV5 VP1 capsid (SEQ ID NO: 1), where heart tropism here refers to properties that are preferred for heart transduction over properties that are preferred for transduction of all other harvested tissues.

TABLE 25
Heart Tropism Rules

Xaa1 is selected from I, K, L, M, T, or V
Xaa1 is selected from K or L
Xaa1 is K
Xaa2 is selected from A, C, G, I, K, or S
Xaa2 is selected from A, C, or S
Xaa2 is A
Xaa3 is selected from A, D, E, G, K, M, or V
Xaa3 is selected from E or V
Xaa3 is E
Xaa4 is selected from F, H, R, T, W, or Y
Xaa4 is selected from F, R, or T
Xaa4 is R
Xaa5 is selected from F, L, M, or R
Xaa5 is L
Xaa6 is selected from A, H, N, W, or Y
Xaa6 is selected from H, N, or Y
Xaa6 is H
Xaa7 is selected from A, C, E, F, K, or T
Xaa7 is selected from C, F, or T
Xaa7 is F
Xaa8 is selected from A, C, M, S, or T
Xaa8 is selected from C, M, or S
Xaa8 is C
Xaa9 is selected from A, D, G, or P
Xaa9 is selected from A or G
Xaa9 is A

TABLE 26 below provides sequences of the 581 to 589 region for variant AAV VP1 capsid polypeptides that were found in heart tissue and comport to one or more of the rules provided in TABLE 25. The present disclosure, thus, provides for rAAVs composed of engineered AAV5 VP1 capsid polypeptides having a SEQ ID NO: 2, wherein the Xaa1 to Xaa9 region is any one of SEQ ID NO: 13118-SEQ ID NO: 14117, as disclosed in TABLE 26. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 26
Sequences of the
581 to 589 Region
in AAV5 VP1 Capsid
Polypeptide that
Drive Heart Tissue
Tropism

| SEQ | |
|---|---|
| ID | 581-589 |
| NO | Sequence |
| 13118 | KAAPMDISI |
| 13119 | KADCPAYKA |
| 13120 | KADVWMAIA |
| 13121 | KAELSDHVL |
| 13122 | KATDALPSC |
| 13123 | KCSKQASYP |
| 13124 | KDGYALLSC |
| 13125 | KDNEAQYQP |
| 13126 | KDYMFNLPF |
| 13127 | KEAMMYNAN |
| 13128 | KEGLIFSTE |
| 13129 | KEHTNICRH |
| 13130 | KEIVEVDGN |
| 13131 | KEMAYQHMW |
| 13132 | KEMLAMWLQ |
| 13133 | KEWFVTWKA |
| 13134 | KFLPCITSY |
| 13135 | KFPYLPHRN |
| 13136 | KGCMSWEEG |
| 13137 | KGDRFCGEV |
| 13138 | KGEPGYMYY |
| 13139 | KGMFVANSH |
| 13140 | KGTKGPQMQ |
| 13141 | KHAGTRYKG |
| 13142 | KHFTSCDRT |
| 13143 | KHHDAAREM |
| 13144 | KHIFNECDC |
| 13145 | KHMQKYVHY |
| 13146 | KHPRFTGWH |
| 13147 | KIESDWHAQ |
| 13148 | KKHQFCEGT |
| 13149 | KLELHGCQN |
| 13150 | KLMSFRELE |
| 13151 | KLQLEKPLN |
| 13152 | KMDCHKYGV |
| 13153 | KMNETTNPR |
| 13154 | KMVDQSLAT |
| 13155 | KMVMIDTDV |
| 13156 | KNFMNHEPQ |
| 13157 | KNNRTIRWG |
| 13158 | KNNTWKWWY |
| 13159 | KNQYFITSQ |
| 13160 | KNWEKQNEV |
| 13161 | KNYNPVNYE |
| 13162 | KPCKACLNS |
| 13163 | KPCWDQAMQ |
| 13164 | KPDQKLWWD |
| 13165 | KPDWWIKVN |
| 13166 | KPSNSKQTV |
| 13167 | KPTQHGVGP |
| 13168 | KQAIGDDVG |
| 13169 | KQHIFQWHH |
| 13170 | KQIHEWEGQ |
| 13171 | KQLPEQQPL |
| 13172 | KQMLEFAPY |
| 13173 | KQNEINHSC |
| 13174 | KRIDHYGWG |
| 13175 | KRSFEWLER |
| 13176 | KSDYNSYDA |
| 13177 | KSLSSEMGV |
| 13178 | KSMDKDLTS |
| 13179 | KSSCVDDSS |
| 13180 | KSVAEGAND |
| 13181 | KTAQLSVME |
| 13182 | KTAYELVDG |
| 13183 | KTCDSQTMT |
| 13184 | KTEMYSDQC |
| 13185 | KTFEWLCPY |
| 13186 | KTMPVGAWD |
| 13187 | KTTWVDDFD |
| 13188 | KTYYVMAIV |
| 13189 | KVLDTLTEH |
| 13190 | KVMHACNAN |
| 13191 | KWDETMFMS |
| 13192 | KWEDFGWWK |
| 13193 | KWEQPNPQA |
| 13194 | KWEYVRCAS |
| 13195 | KWLIARMYL |
| 13196 | KWLKTLMCL |
| 13197 | KWQDAHYIL |
| 13198 | KWTLDAWLT |
| 13199 | KWWVDRLCQ |
| 13200 | KYAVSPCDP |
| 13201 | KYDWLDAGG |
| 13202 | KTDPIAEYC |
| 13203 | KGNPLDGDT |
| 13204 | VAHICDPET |
| 13205 | VASEHSCLP |
| 13206 | VAVEDRPAL |
| 13207 | WAREELGFA |
| 13208 | YAMCHRWMR |
| 13209 | YAPNNSHWP |
| 13210 | AACPHQFKD |
| 13211 | AADLGQSMC |
| 13212 | AAESAKFHE |
| 13213 | AAGIAVACT |
| 13214 | AAIRCMQTS |
| 13215 | AAKLYMDLS |
| 13216 | AAMKGEVIG |
| 13217 | AAMLRLLGK |
| 13218 | AANMLYCST |
| 13219 | AANWTHCSL |
| 13220 | AAPMHLHEI |
| 13221 | AAQFMEKNQ |
| 13222 | AASGLEVVA |
| 13223 | AATNWGVET |
| 13224 | AAVKWDQPL |
| 13225 | AAVPELHHS |
| 13226 | AAVQQPEHT |
| 13227 | AAYSQSAGS |
| 13228 | CADSCYWYP |
| 13229 | CAEAWFPQR |
| 13230 | CAFLDWDWC |
| 13231 | CAFRWVEWD |
| 13232 | CAHHVMWYP |
| 13233 | CAHQPDGYA |
| 13234 | CANAENATC |
| 13235 | CANCVAISP |
| 13236 | CAQPNTHNS |
| 13237 | CAVAWKGDY |
| 13238 | DAAQCTWAR |
| 13239 | DAHPEGPQR |
| 13240 | DALIYHSPF |
| 13241 | DAMRTHENT |
| 13242 | DAQKQQVMH |
| 13243 | DAQWIGCLE |
| 13244 | DARWRSQCP |
| 13245 | DARYSWPGA |
| 13246 | DATFNTSSL |
| 13247 | DAVLFTAMQ |
| 13248 | EAFGNGIDP |
| 13249 | EAGIHQKVI |
| 13250 | EAGRNYMDL |
| 13251 | EANQCHVHI |
| 13252 | EATWTAQGA |
| 13253 | EAYLWENQL |
| 13254 | FAEWKTEQQ |
| 13255 | FAHCAMNES |
| 13256 | FAHWRDCWY |
| 13257 | FAKIRERHV |
| 13258 | FASCMRGRA |
| 13259 | FASPAGPWS |
| 13260 | FAVNEHDVF |
| 13261 | FAYATIRRD |
| 13262 | GAEIMVNGW |
| 13263 | GAEPYVKGG |
| 13264 | GAERQWFTP |
| 13265 | GAHVNSMNA |
| 13266 | GANSSELQQ |
| 13267 | GAVMWERDG |
| 13268 | HADCYVADF |
| 13269 | HADRMEGPW |
| 13270 | HADRPMSAC |
| 13271 | HAEWTEPVD |
| 13272 | HAIPACKRD |
| 13273 | HAKLETMWG |
| 13274 | HALAWCTKH |
| 13275 | HATYCQMAM |
| 13276 | HAVSQERAG |
| 13277 | IACVHMKQW |
| 13278 | IADERWCMA |
| 13279 | IADYEQCSS |
| 13280 | IAFETYWTN |
| 13281 | IAGQWAAFG |
| 13282 | IAKCIDVMD |
| 13283 | IAKWIEKIG |
| 13284 | IAMLSQHTV |
| 13285 | IAQPYDNWG |
| 13286 | IATNHSAGP |
| 13287 | IAVTGFAMS |
| 13288 | LADGMGKVP |
| 13289 | LAFSDITWS |
| 13290 | LAHSEDRTS |
| 13291 | LALQLMGAH |
| 13292 | LAMWFNESC |
| 13293 | LAMWKMRQH |
| 13294 | LATMEDQSN |
| 13295 | MAAPYQPYN |
| 13296 | MAAVMIDQA |
| 13297 | MACRAWIFV |
| 13298 | MADKCANNA |
| 13299 | MAEQAVNSK |
| 13300 | MAGNVCLCV |
| 13301 | MAHMNTGVP |
| 13302 | MAKWDDSFS |
| 13303 | MALMVEECD |
| 13304 | MAMLCSICS |
| 13305 | MANPEMYMQ |
| 13306 | MAQFVWCNG |
| 13307 | MASMMNACN |
| 13308 | MASMSKDDS |
| 13309 | MASQEHQES |
| 13310 | MAVHRPTFC |
| 13311 | MAWDKWFWP |
| 13312 | MAYQQVIAG |
| 13313 | MAYQRHAAF |
| 13314 | NACIRRTTW |
| 13315 | NACIRWAFD |
| 13316 | NADALGALQ |
| 13317 | NADREMFEH |
| 13318 | NAEHRGMTN |
| 13319 | NAGAQWLGI |
| 13320 | NAGLEWKLQ |
| 13321 | NAHCGAACC |
| 13322 | NAMAYENDY |
| 13323 | NAMLTLGAG |
| 13324 | NAMQRANIR |
| 13325 | NAPCFRYYN |
| 13326 | NARDDSIFN |
| 13327 | NASHYEKDG |
| 13328 | NATSYHKFM |
| 13329 | PAEIKMPSQ |
| 13330 | PARLMMWEF |
| 13331 | QADQYYSLE |
| 13332 | QAMLKQGPR |
| 13333 | QASCISWSQ |
| 13334 | QASQENRLT |
| 13335 | QAVECTQAP |
| 13336 | QAYTGDKDI |
| 13337 | RADDYQPVS |
| 13338 | RAELFNNGK |
| 13339 | RAEQADWHE |
| 13340 | RAILNEKRD |
| 13341 | RAINFDKAS |
| 13342 | RAPELVMNE |
| 13343 | RAPYEADSN |
| 13344 | SAAYIVKGP |
| 13345 | SADGQQAAT |
| 13346 | SADLSLTYD |
| 13347 | SADQVPECP |
| 13348 | SAIYRNWFE |
| 13349 | SAKLSEQNV |
| 13350 | SAYALQSPR |
| 13351 | TAAIVGKES |
| 13352 | TACMDHHIR |
| 13353 | TACNMMCAY |
| 13354 | TADWYNGGP |
| 13355 | TAEVWWVFH |
| 13356 | TAGFAKHFP |
| 13357 | TANMCDEGI |
| 13358 | TANSEAHYT |
| 13359 | TARWLDEHL |
| 13360 | TARYITSRA |
| 13361 | TASEFPCGV |
| 13362 | TAWKADSGM |
| 13363 | TAWWNDCWF |
| 13364 | VAAGPLFYG |
| 13365 | VACAPEAMG |
| 13366 | VADGRQPME |
| 13367 | VAFYNKMRG |
| 13368 | VAIQSVGYH |
| 13369 | VAIVWMCSE |
| 13370 | VALPRCQLV |
| 13371 | VAMYTRCHT |
| 13372 | VAPKDNQQM |
| 13373 | VAPQCTNEH |
| 13374 | VAQPVQDMG |
| 13375 | VASKTQFAM |
| 13376 | VAYYFDQAG |
| 13377 | WAPPCYYDV |
| 13378 | YAANKDCFL |
| 13379 | YAKYGQRWM |
| 13380 | YATFEVWFR |
| 13381 | YATMEYNNN |
| 13382 | EAFQKCNWQ |
| 13383 | HATGLVNFM |
| 13384 | AADIFAIND |
| 13385 | EAELENVNM |
| 13386 | IAYHYDQQN |
| 13387 | TSEPLLAML |
| 13388 | TTEDCPNRV |
| 13389 | TTEYTCEDQ |
| 13390 | VFENCSRTF |
| 13391 | VGEWEDEHD |
| 13392 | VIEHAHHVN |
| 13393 | VSEGQVNMD |
| 13394 | VSEHDDCGL |
| 13395 | VSEVYLAER |
| 13396 | WYEKLMDAF |
| 13397 | YCEVPQNPV |
| 13398 | YDEYCESKS |
| 13399 | YIEIVRGPM |
| 13400 | YSEDATPKG |
| 13401 | AFEFTDGCC |
| 13402 | AFESAADHW |
| 13403 | AIEVEGMWD |
| 13404 | ALELEAHRP |
| 13405 | AMERIKMRN |
| 13406 | ATEAYSKLQ |
| 13407 | ATEKDHRVC |
| 13408 | AVEFQHGHC |
| 13409 | AVEIHKCID |
| 13410 | AVEPGISPF |
| 13411 | AYEHDFKCS |
| 13412 | CCEIKRHYY |
| 13413 | CIEKSISST |
| 13414 | CLEQMCRTN |
| 13415 | CPEPFQKVD |
| 13416 | CREQNNCDR |
| 13417 | CSEHGCDIP |
| 13418 | CSEWIRAVR |
| 13419 | CTEQYLVDK |
| 13420 | CTEVRNWYD |
| 13421 | DCEFPMGAN |
| 13422 | DDEGYIFNQ |
| 13423 | DGEFRYVVS |
| 13424 | DGEQESGQT |
| 13425 | DHEVSVAGS |
| 13426 | DIEMCPCYN |
| 13427 | DKEIVNMVW |
| 13428 | DLEYVEFDW |
| 13429 | DQEVFQTHF |
| 13430 | EEEFWMHNA |
| 13431 | EIEPTIGPE |
| 13432 | EKETMWAEP |
| 13433 | ELEKYKLTN |
| 13434 | ELEVRHSND |
| 13435 | EVEEETDEK |
| 13436 | EWEYERGAE |
| 13437 | FCETEPSGA |
| 13438 | FKEHVQNHM |
| 13439 | FQEACMACH |
| 13440 | FSEPSNNFC |
| 13441 | FWESTSPFT |
| 13442 | GEEWVEPTA |
| 13443 | GHEHGWQLS |
| 13444 | GNERLSVCI |
| 13445 | GNETLEMHG |
| 13446 | GRECKQGGH |
| 13447 | GTEHWLSAC |
| 13448 | GYENYNKRD |
| 13449 | HCEYCQMMN |
| 13450 | HFEAGYACS |
| 13451 | HIEGYFRWP |
| 13452 | HIEYTNKCL |
| 13453 | HPEKARPLA |
| 13454 | HPEMKHQNH |
| 13455 | HVENALVFT |
| 13456 | ICEEQFLMG |
| 13457 | IHEEQECGS |
| 13458 | IHEGYKTAP |
| 13459 | IMEQSAQHT |
| 13460 | IRECGQHGS |
| 13461 | ITETWDIFF |
| 13462 | LDEFGFWTH |
| 13463 | LGESEHCMA |
| 13464 | LKEAWICKG |
| 13465 | LKEGINHIC |
| 13466 | LMEGSAKAM |
| 13467 | LYEGRPWYN |
| 13468 | MDEQMGKMS |
| 13469 | MIEKQYPTM |
| 13470 | MKEESKSWP |
| 13471 | MLENYEHMH |
| 13472 | MNEPQKMRQ |
| 13473 | MPEYMKTMQ |
| 13474 | MPEYQARLM |
| 13475 | MREQMPVQY |
| 13476 | MRETVFHEP |
| 13477 | MTESGMKDK |
| 13478 | MVEVSRGEA |
| 13479 | MWECGYGGN |
| 13480 | MWERIGMGG |
| 13481 | MYEDTHCVC |
| 13482 | MYEHINSQR |
| 13483 | MYEKQDGVL |
| 13484 | NGEGSQTCE |
| 13485 | NHEQKSYIF |
| 13486 | NLEIYLQMN |
| 13487 | NVEFMRCNS |
| 13488 | NVEWWAPHV |
| 13489 | NWEQEFVMI |
| 13490 | NWEYMRRKC |
| 13491 | QDERMDLVQ |
| 13492 | QIESGCPHC |
| 13493 | QRELITMGC |
| 13494 | QSEVTHLKW |
| 13495 | QTEQWMCSH |
| 13496 | QWEHIWCWH |
| 13497 | QYENINKVD |
| 13498 | RDEVVFAHC |
| 13499 | RNEPNVWVN |
| 13500 | RTEQSRVWE |
| 13501 | RVEVWYGEL |
| 13502 | SEESVVTHF |
| 13503 | SHEQLAGCP |
| 13504 | SMENRWEYL |
| 13505 | SMEYYYGME |
| 13506 | SNEQMMTMQ |
| 13507 | STECSGLSG |
| 13508 | SYEVTYHMD |
| 13509 | TCEKLISVC |
| 13510 | TKELTRSEA |
| 13511 | TNEAIMQVC |
| 13512 | TNEIGEWWF |
| 13513 | TSELLVNGD |
| 13514 | TSENREYMQ |
| 13515 | TWEKQSRNG |
| 13516 | TWELIQTKF |
| 13517 | VCEEAGELG |
| 13518 | VHEYYRSCA |
| 13519 | VLEFCKDEE |
| 13520 | VMEKQGNGG |
| 13521 | VMEYMLIAW |
| 13522 | VNENNELSA |
| 13523 | VNEQWQIMC |
| 13524 | VNEWSADCS |
| 13525 | VSEFHDLHL |
| 13526 | VSELCTNMA |
| 13527 | VTERWIGPM |
| 13528 | VYEKFSYIT |
| 13529 | VYEREQMDV |
| 13530 | YHECKEGHI |
| 13531 | YSENRIFFR |
| 13532 | YYECLGQDL |
| 13533 | WIEGTLIGA |
| 13534 | NWEFNARHD |
| 13535 | YWEKVPCMK |
| 13536 | GNEDCNLAC |
| 13537 | MNECFSGAQ |
| 13538 | TYESLQNSW |
| 13539 | YCECGHWPN |
| 13540 | CWECCTDSG |
| 13541 | VCPRDMTWE |
| 13542 | VPCRPIYQH |
| 13543 | VRWRGDVAN |
| 13544 | WVLRCTYEY |
| 13545 | ADARVMTMM |
| 13546 | ADLRPSVDY |
| 13547 | AGDRSQILG |
| 13548 | AMMRAWSTF |
| 13549 | AQDRFMLGC |
| 13550 | ATNRIMAAW |
| 13551 | AVVREHVES |
| 13552 | CIWRKYPEF |
| 13553 | CQCRFCHPL |
| 13554 | CTIRVQNGE |
| 13555 | DEFRFLPAD |
| 13556 | DGGRFDDYY |
| 13557 | DHWRKLHKA |
| 13558 | DIARLQVLG |
| 13559 | DKYRQQENS |
| 13560 | DQIRTLQMT |
| 13561 | DRLRDDYPC |
| 13562 | DRPRPMWGH |
| 13563 | DVIRIVHDY |
| 13564 | DYARNHMMW |
| 13565 | DYTRYHSYF |
| 13566 | EGNRPEGLA |
| 13567 | ERVRPLSTD |
| 13568 | ESDREEQSR |
| 13569 | ESWRECMCR |
| 13570 | ETMRCQTMA |
| 13571 | EVARGQDWA |
| 13572 | EVNREETIQ |
| 13573 | EYLRESMMP |
| 13574 | FDDREPFNG |
| 13575 | FDGRPRMFE |
| 13576 | FGPRKNRHE |
| 13577 | FPRRLDHHQ |
| 13578 | FPYRIEGED |
| 13579 | FPYRKVECG |
| 13580 | FTARSYQDA |
| 13581 | FVDRVDKHH |
| 13582 | GNDRAEPDR |
| 13583 | GTCREYGRN |
| 13584 | HGARCCWGV |
| 13585 | HLSRNEYKS |
| 13586 | HSLRDWFKH |
| 13587 | HSPRRATMV |
| 13588 | IHGRMINSI |
| 13589 | IPPRYRHMA |
| 13590 | IRLRDPELH |
| 13591 | IWIRWPSMG |
| 13592 | LFDRVGIEA |
| 13593 | LGCRETRHD |
| 13594 | LIMRNASWC |
| 13595 | LWIRWGSCT |
| 13596 | MHTRIRWLV |
| 13597 | MKCRIEKVN |
| 13598 | MVYRIKMTY |
| 13599 | NFHRWDMNT |
| 13600 | NRDRSWCQH |
| 13601 | NSDRMHCME |
| 13602 | NYPRDHRQH |
| 13603 | NYYRALKWD |
| 13604 | PVTRWEAGS |
| 13605 | QIDRAEKQE |
| 13606 | QNVRSNAYT |
| 13607 | SDLRNLCAR |
| 13608 | SFIRVLRSN |
| 13609 | SIYRMVMQC |
| 13610 | SNDRFPIMC |
| 13611 | SQGRMASGA |
| 13612 | SQTRDTLNF |
| 13613 | SVKRFQHNN |
| 13614 | TQQRVYSIE |
| 13615 | TYDRMQKCN |
| 13616 | VPCRGLVVL |
| 13617 | VPLRHLTDH |
| 13618 | VTDRAFSMN |
| 13619 | VTPRHKTLF |
| 13620 | VTTRVEHFP |
| 13621 | VVMRDEGGR |
| 13622 | WCHRVGCTG |
| 13623 | WDGRFQAAY |
| 13624 | WNLRKANAP |
| 13625 | YHDRFERAH |
| 13626 | YHYREPSAD |
| 13627 | YPGRQHVAN |
| 13628 | YRIRKTCAF |
| 13629 | YVNRYGAGW |
| 13630 | NNVRKVGNT |
| 13631 | TSDWLKHFC |
| 13632 | TWSLLQQGD |
| 13633 | VRAYLMGPN |
| 13634 | VSHALIDWH |
| 13635 | YPKQLHVWT |
| 13636 | ACHTLDQQD |
| 13637 | AHQVLHVRS |
| 13638 | AKRELLDHD |
| 13639 | ANCYLNHTT |
| 13640 | APKKLEHVD |
| 13641 | AQNALWERI |
| 13642 | CFAALDYAS |
| 13643 | CGNALPLIH |
| 13644 | CNDCLMFCD |
| 13645 | CNTMLVSAG |
| 13646 | CSMYLMEFG |
| 13647 | CWYMLKAYT |
| 13648 | DFVQLINRA |
| 13649 | DGYHLEGQC |
| 13650 | DIKALMDGW |
| 13651 | DKSKLDVSY |
| 13652 | DTCKLGGWF |
| 13653 | EHMLLVEQL |
| 13654 | EYNGLGQAY |
| 13655 | FCCLLYARR |
| 13656 | FFSGLEGRH |
| 13657 | FTDQLFTNP |
| 13658 | GGNILDIGP |
| 13659 | GIIPLHWQP |
| 13660 | GSDLLVYSA |
| 13661 | GSMLLGGME |
| 13662 | GTMLLWRQN |
| 13663 | GVANLIGLT |
| 13664 | HELYLTTNT |
| 13665 | HSACLMTMS |
| 13666 | IDTALNVHC |
| 13667 | IENWLFVWL |
| 13668 | IMTMLTSET |
| 13669 | INAVLWWRL |
| 13670 | ITHGLQTWD |
| 13671 | ITTDLQMDH |
| 13672 | IWNELILGC |
| 13673 | LDCELYQTR |
| 13674 | LGMYLQLGQ |
| 13675 | LGVQLSSAP |
| 13676 | LHLKLYGQL |
| 13677 | LLTLLMYWV |
| 13678 | LMYMLPPRH |
| 13679 | LRPWLPDST |
| 13680 | LTHALGTWE |
| 13681 | LWTHLMANH |
| 13682 | MDQKLLTWQ |
| 13683 | MEDMLMEGH |
| 13684 | MHMLLHDVK |
| 13685 | MITWLTDQV |
| 13686 | MNVGLWALW |
| 13687 | MYACLAQPP |
| 13688 | NHCMLHHMG |
| 13689 | NHDHLFERY |
| 13690 | NQPHLWSCG |
| 13691 | NTRDLHTKR |
| 13692 | PNGLLQMVI |
| 13693 | QIAALHQNH |
| 13694 | QLGMLPRAM |
| 13695 | QMVMLKDQY |
| 13696 | QQGQLKTWS |
| 13697 | QVSCLVRDD |
| 13698 | RTWTLECDF |
| 13699 | RVNILEKIA |
| 13700 | SFAHLIDDR |
| 13701 | SIAVLCHGN |
| 13702 | SIDALKSAF |
| 13703 | SIVKLNRSV |
| 13704 | SLNKLFHQE |
| 13705 | SLQFLYHGS |
| 13706 | TERLLQQFY |
| 13707 | TFATLCDQQ |
| 13708 | TIMGLLGNV |
| 13709 | TMYPLMHYS |
| 13710 | TPAQLNGYG |
| 13711 | TQCMLHGPY |
| 13712 | TQGWLTLCY |
| 13713 | TSDWLQNYI |
| 13714 | TTQCLDRWQ |
| 13715 | TTWILPHHD |
| 13716 | TYVPLWAYW |
| 13717 | VEVQLKALQ |
| 13718 | VMHCLRQFM |
| 13719 | VRPWLVMDN |
| 13720 | VTDGLGFRQ |
| 13721 | WVRDLKCPV |
| 13722 | YRIFLMHLC |
| 13723 | CIDVLFKHL |
| 13724 | VFKPLDKRY |
| 13725 | TQGSVHVMN |
| 13726 | VEPTWHCDW |
| 13727 | VISNRHHLP |
| 13728 | WRVSAHYYI |
| 13729 | YDHTIHRNP |
| 13730 | YLLYPHSSG |
| 13731 | ACDLQHNIA |
| 13732 | ADQQQHKVI |
| 13733 | ADWHCHRND |
| 13734 | AEAQFHTGE |
| 13735 | ANHWKHEWE |
| 13736 | ASGWGHCWR |
| 13737 | AVIKAHCAA |
| 13738 | AYCHQHQHA |
| 13739 | AYMPYHKEQ |
| 13740 | AYQAEHVGM |
| 13741 | CINWKHVAS |
| 13742 | DDHWHHRAK |
| 13743 | DFLTAHCIE |
| 13744 | DFLYMHRMG |
| 13745 | DGDAAHTKP |
| 13746 | DHQLCHVAL |
| 13747 | DHVDNHEEA |
| 13748 | DNAYFHIKG |
| 13749 | DPCNHHWCI |
| 13750 | DVVYTHCIN |
| 13751 | EENHSHDER |
| 13752 | EINFQHRHV |
| 13753 | EIQWNHWYV |
| 13754 | EKDMHHVAS |
| 13755 | ELLKVHGPK |
| 13756 | EMNGRHWMP |
| 13757 | EPAVTHDSA |
| 13758 | EQHIMHEQW |
| 13759 | ERMVCHWDA |
| 13760 | EWPINHQKV |
| 13761 | FDKAFHVHM |
| 13762 | FKPIVHHCG |
| 13763 | GEDQIHVWH |
| 13764 | GHQSEHRCS |
| 13765 | GINSHHNDL |
| 13766 | GKQNNHRIE |
| 13767 | GNKYMHHHA |
| 13768 | GPKPVHFDV |
| 13769 | GSVMGHCGS |
| 13770 | GWGCGHGVH |
| 13771 | HGYNWHHLG |
| 13772 | HTLHRHWFW |
| 13773 | HTQFAHGVD |
| 13774 | HVAIGHGGF |
| 13775 | IERHEHNPY |
| 13776 | IIPHFHLCY |
| 13777 | IKCIQHVHY |
| 13778 | IKDITHNHP |
| 13779 | LCRQTHKWV |
| 13780 | LFGVYHFSG |
| 13781 | LICKYHYCQ |
| 13782 | LQPMMHTHS |
| 13783 | LTLCMHEAD |
| 13784 | MCGLYHTEG |
| 13785 | MCHGCHRSY |
| 13786 | MFDNCHVFP |
| 13787 | MNDTYHRRA |
| 13788 | MNLTIHEQF |
| 13789 | MQMFKHNWI |
| 13790 | MTGVEHVFY |
| 13791 | NCFWTHVSG |
| 13792 | NDYPIHCYS |
| 13793 | NFTSFHTYS |
| 13794 | NHMLTHHYH |
| 13795 | NYAFNHQGV |
| 13796 | NYQQHHFFS |
| 13797 | QHSWKHFDS |
| 13798 | QILHYHYHG |
| 13799 | QMNWKHVWN |
| 13800 | QNSERHDEP |
| 13801 | QRMFTHQEY |
| 13802 | QSFSPHFGV |
| 13803 | QSVHTHANL |
| 13804 | QTGNAHWQE |
| 13805 | RCNEDHEQD |
| 13806 | RIIEQHAHG |
| 13807 | RQCEMHTVD |
| 13808 | SCGWKHDNP |
| 13809 | SCTEMHAGR |
| 13810 | SLYVHHKDG |
| 13811 | STGLQHDFN |
| 13812 | TEGQTHHTA |
| 13813 | TERSAHVTY |
| 13814 | TFKWMHEGW |
| 13815 | TFNNCHMQS |
| 13816 | THTQQHVVN |
| 13817 | TMSDRHICH |
| 13818 | TNATSHWCG |
| 13819 | TNGWMHHWD |
| 13820 | TTMIRHHGH |
| 13821 | TVFGTHRAL |
| 13822 | TVGMFHCQG |
| 13823 | TYTPRHRFS |
| 13824 | VRKWQHHGG |
| 13825 | VTTHCHHTY |
| 13826 | WEAKWHAWE |
| 13827 | WGFNDHKRG |
| 13828 | WRGIGHADL |
| 13829 | WTTAFHKRT |
| 13830 | YIVQTHCGG |
| 13831 | YKCHDHCHD |
| 13832 | YNNFHHQMY |
| 13833 | YRNEPHQGF |
| 13834 | YTNYKHCCF |
| 13835 | TGCNHHGRF |
| 13836 | DVCDDHKPN |
| 13837 | DVAPMHQVE |
| 13838 | VFKPNSFQT |
| 13839 | VPIQFSFAS |
| 13840 | VPLWDVFQT |
| 13841 | VTGVTIFEL |
| 13842 | YTPTRYFGK |
| 13843 | YVGWHMFLD |
| 13844 | IWKIQEFGT |
| 13845 | AGQPVTFHF |
| 13846 | AHASHMFGG |
| 13847 | ARQMICFGG |
| 13848 | ATYYNQFHD |
| 13849 | CCTWKCFCQ |
| 13850 | DFPLFTFGN |
| 13851 | DIRHMSFEF |
| 13852 | DPSQPGFNA |
| 13853 | DVAHKCFIS |
| 13854 | EGGKERFQQ |
| 13855 | EKMTWVFAG |
| 13856 | ENTTCNFQG |
| 13857 | ESLGCEFML |
| 13858 | FGCCGRFVI |
| 13859 | FTGKRDFMK |
| 13860 | FTMWHRFAW |
| 13861 | GDNAKDFRG |
| 13862 | GHAPHLFAD |
| 13863 | GIRSTDFYQ |
| 13864 | GMIVDVFWV |
| 13865 | GMTTMAFQG |
| 13866 | HGAKAPFTA |
| 13867 | HNDYPQFQP |
| 13868 | HVNAGSFGS |
| 13869 | IFILRTFFA |
| 13870 | IFPWFKFHW |
| 13871 | IQVHSLFGC |
| 13872 | IRPFIEFLQ |
| 13873 | LHQKPNFDC |
| 13874 | LIHHCTFEQ |
| 13875 | LQLEDRFNG |
| 13876 | MCSHYQFMS |
| 13877 | MFYLEDFRQ |
| 13878 | MITQFIFNI |
| 13879 | MTVMQGFLS |
| 13880 | NPQWGLFNQ |
| 13881 | NTGARPFAG |
| 13882 | PCLQTRFAQ |
| 13883 | PMYQDMFTL |
| 13884 | QGDLMGFHY |
| 13885 | QIGMNTFMS |
| 13886 | QMHVMIFGS |
| 13887 | REWDHYFQI |
| 13888 | RLRISPFNI |
| 13889 | RMGAWSFPS |
| 13890 | RVTTAEFLC |
| 13891 | RWSQNKFQR |
| 13892 | SDHDNVFED |
| 13893 | SHTTYDFFK |
| 13894 | SNVCNSFVN |
| 13895 | SPHAGPFGC |
| 13896 | SQFMMDFGP |
| 13897 | SQVPGRFIF |
| 13898 | TCCLYEFIH |
| 13899 | TGGTAEFGL |
| 13900 | TSNWHEFTN |
| 13901 | TSPCCTFGI |
| 13902 | VHDAQGFCA |
| 13903 | VKMMWDFAD |
| 13904 | VTHITEFMA |
| 13905 | VVSTDMFIH |
| 13906 | WCAPAVFNE |
| 13907 | YFREKPFDV |
| 13908 | YQCKMAFHV |
| 13909 | GVWMQGFHP |
| 13910 | MWGCKLFVC |
| 13911 | YLDSSECCW |
| 13912 | YVKPCYDCI |
| 13913 | AGLQHDTCV |
| 13914 | AITHVMHCH |
| 13915 | ASVLQQLCV |
| 13916 | ATTGMLACH |
| 13917 | AVGATMMCH |
| 13918 | AVHDNFWCL |
| 13919 | CCKYNQLCH |
| 13920 | CDKIDPYCD |
| 13921 | CDTDESGCY |
| 13922 | CHRCKVNCQ |
| 13923 | CITPYRPCD |
| 13924 | CMYSWVPCG |
| 13925 | CVHYCFGCS |
| 13926 | CVKSYEKCF |
| 13927 | DEHAHWSCS |
| 13928 | DEHQMMECG |
| 13929 | DIGFRIDCD |
| 13930 | DMCEFMYCD |
| 13931 | DMFYWQPCE |
| 13932 | DRRDHEACN |
| 13933 | EFDSPNQCY |
| 13934 | EFHPHDWCL |
| 13935 | ERGFMEHCH |
| 13936 | ESMSTRACG |
| 13937 | ETDPYKSCH |
| 13938 | ETVSPVGCH |
| 13939 | EVAQTMRCA |
| 13940 | EYQLSETCT |
| 13941 | FHKCYSACR |
| 13942 | FKDMIARCP |
| 13943 | GDDCKFDCL |
| 13944 | GTGSTWECT |
| 13945 | GVAVTTCCR |
| 13946 | HSNMRSACE |
| 13947 | IKRTYPACW |
| 13948 | IMCECQHCE |
| 13949 | IPFPMCGCC |
| 13950 | IRAPFNRCE |
| 13951 | ITCTYRNCE |
| 13952 | ITDYQSDCY |
| 13953 | LCALHEQCQ |
| 13954 | LSVSSDACY |
| 13955 | LVVAHCGCV |
| 13956 | MDAYFNSCQ |
| 13957 | MGTFVAHCY |
| 13958 | MIALNQNCT |
| 13959 | MMPSYTLCW |
| 13960 | MPAWMMNCH |
| 13961 | MQQCDGGCG |
| 13962 | MSWSVYWCI |
| 13963 | MTNCIEYCA |
| 13964 | MTTLYQSCL |
| 13965 | MVHGALMCR |
| 13966 | MVRSREQCI |
| 13967 | NGGKGQQCD |
| 13968 | NLGYCEHCY |
| 13969 | NLMTPWPCQ |
| 13970 | NSNMMAHCE |
| 13971 | NWVPVYDCH |
| 13972 | PFILSNCCA |
| 13973 | QNGVWENCE |
| 13974 | QSDGFEDCS |
| 13975 | QTTNNQRCD |
| 13976 | RDTKRSCCH |
| 13977 | RFVDFGSCQ |
| 13978 | RHYYWVLCP |
| 13979 | RNMYFVDCM |
| 13980 | RYDKVCYCV |
| 13981 | SGFSEKCCK |
| 13982 | SGRPKLVCN |
| 13983 | SHDPHNTCG |
| 13984 | SIGYYCHCM |
| 13985 | SILPTWGCD |
| 13986 | SKHDFDHCF |
| 13987 | THILYSWCT |
| 13988 | TIFYCRWCN |
| 13989 | TKNEYGLCF |
| 13990 | TVQYGRGCS |
| 13991 | TWGLKWMCP |
| 13992 | VDMVVDNCC |
| 13993 | VFGDGAHCI |
| 13994 | VHDQRLVCD |
| 13995 | VNPCAQGCH |
| 13996 | VNPQFSQCT |
| 13997 | VPMYTQVCN |
| 13998 | VSALIDHCC |
| 13999 | VTGPMPTCL |
| 14000 | VTMQVRQCA |
| 14001 | VVMQKIGCI |
| 14002 | VVNTVGCCV |
| 14003 | WHLFSACCS |
| 14004 | WHMLCDGCS |
| 14005 | WMRTQCWCP |
| 14006 | YGGLSCGCL |
| 14007 | YGPSTEPCQ |
| 14008 | LSAQRLVCD |
| 14009 | WVHPYSYCA |
| 14010 | TYHDIIVTA |
| 14011 | VPNVQNMKA |
| 14012 | VWAWQNGYA |
| 14013 | AEAIWLPWA |
| 14014 | AECLGYVDA |
| 14015 | AHVVHTSGA |
| 14016 | ALLFAPARA |
| 14017 | AMPNGLDVA |
| 14018 | ARLCTWPYA |
| 14019 | ATNKNCAHA |
| 14020 | CCFQNNTQA |
| 14021 | CLNGWKQAA |
| 14022 | CSQSYAADA |
| 14023 | CTNHNDHDA |
| 14024 | DFIMMQDQA |
| 14025 | DPLVHTMIA |
| 14026 | DRDNVDSQA |
| 14027 | DRSEMGAYA |
| 14028 | DRSVVDVDA |
| 14029 | DTDMIAQFA |
| 14030 | DTWKVMMAA |
| 14031 | EGQSMMYQA |
| 14032 | EIVGQKGWA |
| 14033 | EKQLYMYEA |
| 14034 | ELTIIMVVA |
| 14035 | ERDGWNMAA |
| 14036 | ESSLFEHYA |
| 14037 | ETQWWDVIA |
| 14038 | FVIPSGAGA |
| 14039 | GGHNYGMHA |
| 14040 | GIAASCHLA |
| 14041 | GSCSTLTSA |
| 14042 | GSLPYSMYA |
| 14043 | GTMIYSHMA |
| 14044 | HHGQCGGMA |
| 14045 | HTFLCNITA |
| 14046 | IISAKQHDA |
| 14047 | IKCIPGWYA |
| 14048 | ILAQANIHA |
| 14049 | ILHSVTVRA |
| 14050 | INRQCDMIA |
| 14051 | IQKYGAEVA |
| 14052 | IQMLVQGSA |
| 14053 | IRMHMPRDA |
| 14054 | ISKPYQLTA |
| 14055 | ITKCDGNTA |
| 14056 | IVTASVCQA |
| 14057 | LNSLVCSFA |
| 14058 | LTKIVTTGA |
| 14059 | LWCVSNSFA |
| 14060 | MEIGIMKIA |
| 14061 | MERIMLSLA |
| 14062 | MGWYIVRHA |
| 14063 | MHNWGGKIA |
| 14064 | MHWLKYNYA |
| 14065 | MMAHSGHMA |
| 14066 | MMDPCNPPA |
| 14067 | MNGMPTSFA |
| 14068 | MPDSWVRVA |
| 14069 | MRIAQCNGA |
| 14070 | MSMQHLEHA |
| 14071 | MSTHEDHVA |
| 14072 | MVNTGGNTA |
| 14073 | MVTCVQSGA |
| 14074 | MWMMQGMNA |
| 14075 | NCDQMSISA |
| 14076 | NCKLESDLA |
| 14077 | NDMYVKGTA |
| 14078 | NINGIINQA |
| 14079 | NRTDRMMDA |
| 14080 | NTCQHVNFA |
| 14081 | PCSTWLLAA |
| 14082 | QKMFGDSYA |
| 14083 | QNMSNWLDA |
| 14084 | QQVCDIPSA |
| 14085 | QSTKEYWQA |
| 14086 | QVMCMCAAA |
| 14087 | RTNLQQVDA |
| 14088 | SCAQERHDA |
| 14089 | SILTGGGFA |
| 14090 | SLVCHRNFA |
| 14091 | SMDVHCNDA |
| 14092 | SMKLISEAA |
| 14093 | SMNATRWKA |
| 14094 | SQACSNHYA |
| 14095 | SYMLKSDYA |
| 14096 | THVNRARWA |
| 14097 | TRCTCERYA |
| 14098 | TTDACWQHA |
| 14099 | TTHHAQAGA |
| 14100 | TTSMGKHYA |
| 14101 | TVGLDAHSA |
| 14102 | TYWPGCANA |
| 14103 | VCTAINDNA |
| 14104 | VIDTIAGHA |
| 14105 | VLSLFKEPA |
| 14106 | VNQQIESDA |
| 14107 | VTQLVTRLA |
| 14108 | VTTQIKRIA |
| 14109 | VWHVPRDQA |
| 14110 | VYCGNKTKA |
| 14111 | YDAKSSVNA |
| 14112 | YGREKQAYA |
| 14113 | YVHADPEFA |
| 14114 | YVSQKEAIA |
| 14115 | YWLEVDSKA |
| 14116 | DVQGSGQWA |
| 14117 | SVLHVQALA |

### Example 14

AAV5 Variants with Tissue Tropism in Colon

This example describes engineered AAV5 variants with tissue tropism in colon that were discovered using the methods and systems described in EXAMPLE 1-EXAMPLE 2.

CNS, liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive colon tropism. The results are shown in FIG. 18M which shows the positional frequency of each amino acid in the 581 to 589 region of AAV5 VP1 in colon tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in colon over the frequency of that given amino acid residue occurring at the specified position in variants identified in all other harvested tissues was analyzed to identify a set of sequence rules for capsids that preferentially target colon tissue. With reference to TABLE 27 below, and SEQ ID NO: 2 (AAV5 VP1), the following amino acids can, thus, be independently mutated, in any combination, at any one or more positions Xaa1-Xaa9, to provide a VP1 capsid with enhanced colon tropism as compared wildtype AAV5 VP1 capsid (SEQ ID NO: 1), where colon tropism here refers to properties that are preferred for colon transduction over properties that are preferred for transduction of all other harvested tissues.

TABLE 27
Colon Tropism Rules

Xaa1 is selected from C, F, H, N, P, W, or Y
Xaa1 is selected from F, P, or W
Xaa1 is P
Xaa2 is selected from D, E, F, L, or P
Xaa2 is selected from D, E, L, or P
Xaa2 is P
Xaa3 is selected from C, F, H, I, L, P, or Y
Xaa3 is selected from C, H, or P
Xaa3 is P
Xaa4 is selected from C, D, E, N, or P
Xaa4 is selected from C, D, or E
Xaa4 is C
Xaa5 is selected from D, E, G, P, or W
Xaa5 is selected from G, P, or W
Xaa5 is P
Xaa6 is selected from C, K, R, or V
Xaa6 is selected from K or R
Xaa6 is R
Xaa7 is selected from D, M, P, or V
Xaa7 is P
Xaa8 is selected from D, I, K, L, P, R, or V
Xaa8 is selected from K, P, or R
Xaa8 is P
Xaa9 is selected from C, H, I, K, L, M, or W
Xaa9 is selected from I, L, or M
Xaa9 is I

TABLE 28 below provides sequences of the 581 to 589 region for variant AAV VP1 capsid polypeptides that were found in colon tissue and comport to one or more of the rules provided in TABLE 27. The present disclosure, thus, provides for rAAVs composed of engineered AAV5 VP1 capsid polypeptides having a SEQ ID NO: 2, wherein the Xaa1 to Xaa9 region is any one of SEQ ID NO: 10118-SEQ ID NO: 11117, as disclosed in TABLE 28. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 28
Sequences of the
581 to 589 Region
in AAV5 VP1 Capsid
Polypeptide that
Drive Colon Tissue
Tropism

| SEQ | |
|---|---|
| ID | 581-589 |
| NO | Sequence |
| 10118 | PENIAEDHE |
| 10119 | PYALAEGNP |
| 10120 | PLAGRFVAQ |
| 10121 | PAEAKQRFD |
| 10122 | PAAYDGRLL |
| 10123 | PACHCYESL |
| 10124 | PACRFEANS |
| 10125 | PACSPYGWW |
| 10126 | PADFVNSNY |
| 10127 | PAECWRMTF |
| 10128 | PAEFDYLFT |
| 10129 | PAHEIKNLE |
| 10130 | PAIDHLLHR |
| 10131 | PAMQGHMVD |
| 10132 | PAMRTVHMW |
| 10133 | PAQWAWADG |
| 10134 | PARCAVSEH |
| 10135 | PASLKEHCV |
| 10136 | PATCSQKKY |
| 10137 | PATPPTIVG |
| 10138 | PAYHTWQYN |
| 10139 | PCAPYHPNQ |
| 10140 | PCDCSYYFR |
| 10141 | PCGSDIGCM |
| 10142 | PCHMWMNFY |
| 10143 | PCIWNWETD |
| 10144 | PCKPYPKDL |
| 10145 | PCNNCGHIR |
| 10146 | PCPMSPGEW |
| 10147 | PCQLKSDGE |
| 10148 | PCRDTLEGI |
| 10149 | PCRPLPHLL |
| 10150 | PCSMPKAGG |
| 10151 | PCTVEGRQY |
| 10152 | PCYMKSWVM |
| 10153 | PDCQEFWKQ |
| 10154 | PDGFSCRFA |
| 10155 | PDHCLQSTK |
| 10156 | PDHRDEMEW |
| 10157 | PDHTNYHIV |
| 10158 | PDIIIEGNT |
| 10159 | PDINYSCAI |
| 10160 | PDLRTQLFS |
| 10161 | PDMCGRMDM |
| 10162 | PDMQTEAGA |
| 10163 | PDPRRTNWQ |
| 10164 | PDPVDHRGD |
| 10165 | PDPWSTSHK |
| 10166 | PDQQYELSC |
| 10167 | PDRNLSQGP |
| 10168 | PDRWCQLYK |
| 10169 | PDSHVPLRD |
| 10170 | PDTKYLHRC |
| 10171 | PDVQNRWEM |
| 10172 | PDVTTNNVI |
| 10173 | PDWLMWSHH |
| 10174 | PDWYHCGFQ |
| 10175 | PEAEFGQKS |
| 10176 | PEAEYEQMK |
| 10177 | PECHNYNQI |
| 10178 | PEDENPLYE |
| 10179 | PEDQHNTWC |
| 10180 | PEDWKRHAN |
| 10181 | PEEQTCEGR |
| 10182 | PEFPTWGHY |
| 10183 | PEGLWSVAC |
| 10184 | PEHKGYAGW |
| 10185 | PEISHKNVY |
| 10186 | PEKESTVIS |
| 10187 | PEKYCHEGY |
| 10188 | PELAMPWEM |
| 10189 | PELTYAKSI |
| 10190 | PEMCKMGSP |
| 10191 | PENWFWPHQ |
| 10192 | PEPSAMTIP |
| 10193 | PEQLLYSMK |
| 10194 | PEREDCHNP |
| 10195 | PESEGRMTK |
| 10196 | PESKLMWGA |
| 10197 | PETKTFCQQ |
| 10198 | PEWTTTTVF |
| 10199 | PFACGMSTW |
| 10200 | PFAEFVGRT |
| 10201 | PFAGGEYDG |
| 10202 | PFCIPWTEG |
| 10203 | PFCQPPEFN |
| 10204 | PFDRMDHLA |
| 10205 | PFGLSQCKG |
| 10206 | PFGQLKFNM |
| 10207 | PFHVPPPTH |
| 10208 | PFKCQDIDC |
| 10209 | PFMSTYQVE |
| 10210 | PFPVQNVDS |
| 10211 | PFSKWVYEW |
| 10212 | PFVSQWEHM |
| 10213 | PFWWVCKVI |
| 10214 | PGGYKEHTW |
| 10215 | PGIPIVWMH |
| 10216 | PGKQTLTTQ |
| 10217 | PGMMPGCTA |
| 10218 | PGPFRGPER |
| 10219 | PGVTCFSQT |
| 10220 | PHCLKAGPL |
| 10221 | PHDARVSRM |
| 10222 | PHEDNNHQT |
| 10223 | PHELCHISH |
| 10224 | PHENKFLDC |
| 10225 | PHHGANGFC |
| 10226 | PHHWCIVES |
| 10227 | PHKCMFSWN |
| 10228 | PHNYRVFWE |
| 10229 | PHPPNPGEN |
| 10230 | PHQHPQIPE |
| 10231 | PHRDVIWLG |
| 10232 | PHRVNVERS |
| 10233 | PHSNSVGKC |
| 10234 | PHSQKLNSG |
| 10235 | PHTDAGYIH |
| 10236 | PHTQALICP |
| 10237 | PIASEMYED |
| 10238 | PICEFCTMN |
| 10239 | PIEFYTIKH |
| 10240 | PIEGYHIAD |
| 10241 | PIESKPRSS |
| 10242 | PIFPPGQKP |
| 10243 | PIHHQLMCF |
| 10244 | PIIPCGGWQ |
| 10245 | PIISTENNQ |
| 10246 | PILCTMPFV |
| 10247 | PILIPSLQP |
| 10248 | PILPNTHTG |
| 10249 | PIMCKEPGV |
| 10250 | PIMMHQTLG |
| 10251 | PIMPKYNAL |
| 10252 | PINTSHSYF |
| 10253 | PIPHVMNEP |
| 10254 | PIRQNAMMW |
| 10255 | PITLRQPWH |
| 10256 | PIVAWIWYD |
| 10257 | PIVHQNPMN |
| 10258 | PIVVDGRKF |
| 10259 | PIYSIVTKG |
| 10260 | PKCEILTGA |
| 10261 | PKGADRYNY |
| 10262 | PKGVAMGGT |
| 10263 | PKIEPNLCK |
| 10264 | PKIMPKSCS |
| 10265 | PKLEDDEHC |
| 10266 | PKLTCETWF |
| 10267 | PKSDLCEWV |
| 10268 | PKSELQKSF |
| 10269 | PKSIDELFE |
| 10270 | PKTWMHGVP |
| 10271 | PLCLKYEGC |
| 10272 | PLECNRGTD |
| 10273 | PLEMSNTPW |
| 10274 | PLGFAPEIT |
| 10275 | PLHQTSEYK |
| 10276 | PLLMTKTGS |
| 10277 | PLMMGPQRQ |
| 10278 | PLNHACYCC |
| 10279 | PLNWIIACR |
| 10280 | PLQTSKWQA |
| 10281 | PLVKLEEIH |
| 10282 | PMCICWASE |
| 10283 | PMFYDEPKH |
| 10284 | PMGCNGYVT |
| 10285 | PMGYSNLNC |
| 10286 | PMHPTCFVI |
| 10287 | PMIVPDVAY |
| 10288 | PMLKTEAMS |
| 10289 | PMLPEKFPD |
| 10290 | PMMISCNSV |
| 10291 | PMMWSTNVK |
| 10292 | PMNWAHSCA |
| 10293 | PMPNYYHPQ |
| 10294 | PMRPHRDAE |
| 10295 | PMSPFPFEH |
| 10296 | PMTSTEPME |
| 10297 | PMWYYYHRM |
| 10298 | PMYSQVHLG |
| 10299 | PMYVLEAHY |
| 10300 | PNAKPCCFE |
| 10301 | PNCLKSIHG |
| 10302 | PNCPFYPHP |
| 10303 | PNDLNRRTK |
| 10304 | PNEPMTPGQ |
| 10305 | PNESGHQSS |
| 10306 | PNFYSDISR |
| 10307 | PNIKYVYNG |
| 10308 | PNIPLIDMC |
| 10309 | PNMPTCNAP |
| 10310 | PNQYTANEI |
| 10311 | PNRHGPDAE |
| 10312 | PNSSTPYNW |
| 10313 | PNWQNLESD |
| 10314 | PPDAFRWCP |
| 10315 | PPDIQPNCS |
| 10316 | PPELEKCTP |
| 10317 | PPFCKWYIV |
| 10318 | PPILLIQMR |
| 10319 | PPKDKYLRE |
| 10320 | PPKPAEEGH |
| 10321 | PPKYPQQQI |
| 10322 | PPPSVRMMM |
| 10323 | PPQQCEWIA |
| 10324 | PPRHSNSQG |
| 10325 | PPTIGCSCN |
| 10326 | PQAEVMLGE |
| 10327 | PQCRMLLWE |
| 10328 | PQDDKGVIP |
| 10329 | PQECSHVHP |
| 10330 | PQKLNFTYC |
| 10331 | PQKTLAVGN |
| 10332 | PQLVSHIGH |
| 10333 | PQNPKMIPA |
| 10334 | PQNTFVRKQ |
| 10335 | PQPCSYDSG |
| 10336 | PQRYIFGAW |
| 10337 | PQSCENNCR |
| 10338 | PQSHCHMET |
| 10339 | PQTFWGETC |
| 10340 | PQVMQLAEA |
| 10341 | PQYIQAIED |
| 10342 | PQYRCIDEH |
| 10343 | PQYTNRVIG |
| 10344 | PQYVPDRGT |
| 10345 | PRCLKCRPH |
| 10346 | PRGANSMMT |
| 10347 | PRLFMREID |
| 10348 | PRLNVELDW |
| 10349 | PRLQWMEPG |
| 10350 | PRMCMSENE |
| 10351 | PRMWDKLDQ |
| 10352 | PRRDKDVYM |
| 10353 | PRVCVTWVT |
| 10354 | PRWMQTYQV |
| 10355 | PSACMVGPC |
| 10356 | PSADCNPWA |
| 10357 | PSAGDLSQY |
| 10358 | PSAQGNFSM |
| 10359 | PSCVIFKPW |
| 10360 | PSDHTWKNV |
| 10361 | PSEGDSWLC |
| 10362 | PSESLELCP |
| 10363 | PSFLCGKYI |
| 10364 | PSGCYKCMK |
| 10365 | PSKHKDLNE |
| 10366 | PSLYCCDGV |
| 10367 | PSMYEDMTN |
| 10368 | PSNPEQHLV |
| 10369 | PSPRYPVCM |
| 10370 | PSVLQTTFN |
| 10371 | PSVTVDVGS |
| 10372 | PTCLGKIKW |
| 10373 | PTCQTPPLH |
| 10374 | PTEFDQEQT |
| 10375 | PTEPREDAA |
| 10376 | PTETRWHTG |
| 10377 | PTFMFRRDL |
| 10378 | PTGIYAMQC |
| 10379 | PTHLFNFHW |
| 10380 | PTINRYFNE |
| 10381 | PTKEMMSMF |
| 10382 | PTKSSGGAA |
| 10383 | PTMLAVKNA |
| 10384 | PTMPWKKAE |
| 10385 | PTMYRPMHV |
| 10386 | PTNGIASAH |
| 10387 | PTPSIVLCQ |
| 10388 | PTQYIGDHR |
| 10389 | PTRIQMTVD |
| 10390 | PTVWANVAE |
| 10391 | PTVWRTFEF |
| 10392 | PTWYVLAHF |
| 10393 | PTYPFISYS |
| 10394 | PVAGDRYGG |
| 10395 | PVAVPTESP |
| 10396 | PVDRLVHTN |
| 10397 | PVEMPDSCH |
| 10398 | PVGSHMTFW |
| 10399 | PVIHGMHID |
| 10400 | PVIKPNTEV |
| 10401 | PVIQQRRDQ |
| 10402 | PVIRKIYEL |
| 10403 | PVKCEDHPE |
| 10404 | PVKSPWFNP |
| 10405 | PVLCEMKQY |
| 10406 | PVNWPPFFC |
| 10407 | PVPRRKCSS |
| 10408 | PVPTFCSTC |
| 10409 | PVPYETHMY |
| 10410 | PVRKAGWQK |
| 10411 | PVSPPCDAQ |
| 10412 | PVVMIGHMS |
| 10413 | PWDLRNQYH |
| 10414 | PWEAMYMTA |
| 10415 | PWERRHAER |
| 10416 | PWETCRWKQ |
| 10417 | PWIAMVAQE |
| 10418 | PWNQKCNYP |
| 10419 | PWNWDGHMR |
| 10420 | PWQCLRSPD |
| 10421 | PWVRWRTCQ |
| 10422 | PYAANHQQG |
| 10423 | PYACKIIRG |
| 10424 | PYCNIWMES |
| 10425 | PYCPMQIDC |
| 10426 | PYCWMVDDA |
| 10427 | PYEHGKEHH |
| 10428 | PYGYNCVID |
| 10429 | PYHRIMEYA |
| 10430 | PYIRCKKDG |
| 10431 | PYMRCEHQH |
| 10432 | PYMVTCYDF |
| 10433 | PYNPQYCCC |
| 10434 | PYPWDRCWI |
| 10435 | PYRKPWNAE |
| 10436 | PYRVEGEHN |
| 10437 | PYSFKDLLE |
| 10438 | PYTIQKGEY |
| 10439 | PYTNEQQYT |
| 10440 | PYVQAQAGR |
| 10441 | PYVRRVTEI |
| 10442 | MPTRQRHEK |
| 10443 | APILLQASC |
| 10444 | DPRRSMWYL |
| 10445 | IPRIVYMCD |
| 10446 | SPCNMDTHH |
| 10447 | RPVTYPHTA |
| 10448 | NPGWTLSMA |
| 10449 | IPSHVHFGE |
| 10450 | APDNEAGMY |
| 10451 | APDPSTANR |
| 10452 | APFNANCCN |
| 10453 | APGCYFYRN |
| 10454 | APICEGEPK |
| 10455 | APITIGAMS |
| 10456 | APKWFNSLD |
| 10457 | APLHNFPVT |
| 10458 | APMDMGRMT |
| 10459 | APMHSFDAE |
| 10460 | APNDCMMQN |
| 10461 | APPHQDHNP |
| 10462 | APQCVIVFT |
| 10463 | APRQIWMHH |
| 10464 | APVYWDHQM |
| 10465 | APWWEFPQY |
| 10466 | APYDPKYVF |
| 10467 | APYQAVMES |
| 10468 | CPALNQLGR |
| 10469 | CPAMPGYCH |
| 10470 | CPDDSRLDQ |
| 10471 | CPDLPDWSG |
| 10472 | CPFFNYMIQ |
| 10473 | CPGYYETEI |
| 10474 | CPHAHYVWR |
| 10475 | CPIYYGPVS |
| 10476 | CPKGHEYEN |
| 10477 | CPKQWLPAC |
| 10478 | CPLIDWCMY |
| 10479 | CPLPARWGD |
| 10480 | CPNCWYDEV |
| 10481 | CPPQSENKV |
| 10482 | CPPRRYVNG |
| 10483 | CPQCMQGAS |
| 10484 | CPQMPWLHF |
| 10485 | CPRQLYHWQ |
| 10486 | CPSCEISVP |
| 10487 | CPSECDNLK |
| 10488 | CPSKQTRNP |
| 10489 | CPTQLYLHN |
| 10490 | CPVCEDMPL |
| 10491 | CPVLFTHME |
| 10492 | CPWLELCYH |
| 10493 | CPYCDQEGH |
| 10494 | DPAGDLMQG |
| 10495 | DPCNVDPGF |
| 10496 | DPCTCHTAK |
| 10497 | DPDPVDMYA |
| 10498 | DPGAPGPKA |
| 10499 | DPHCIPTWK |
| 10500 | DPHCVKNNC |
| 10501 | DPICVVWTG |
| 10502 | DPIHAHVEC |
| 10503 | DPKHYNETM |
| 10504 | DPKICWQER |
| 10505 | DPKSCPRRE |
| 10506 | DPKSGFNGT |
| 10507 | DPMCFHAYQ |
| 10508 | DPMQMHRSG |
| 10509 | DPPKCYRYE |
| 10510 | DPPNTSFFH |
| 10511 | DPPRCDMQK |
| 10512 | DPPSPVMCG |
| 10513 | DPQLFCSDL |
| 10514 | DPRMGGSVN |
| 10515 | DPSYNERHR |
| 10516 | EPCHWMHEF |
| 10517 | EPDQMQERL |
| 10518 | EPDQPRWDS |
| 10519 | EPECTEFKV |
| 10520 | EPFPHVMWG |
| 10521 | EPGIVHWYD |
| 10522 | EPGYYDGGN |
| 10523 | EPHKYNQPS |
| 10524 | EPHLQPHQK |
| 10525 | EPHLTAVPT |
| 10526 | EPKNYSERH |
| 10527 | EPLKYNHES |
| 10528 | EPLQFMELM |
| 10529 | EPMRLGEWS |
| 10530 | EPPVCQGNE |
| 10531 | EPRRVDDQQ |
| 10532 | EPVMVRMQF |
| 10533 | FPALIKPHE |
| 10534 | FPASPDILI |
| 10535 | FPCGIVWTH |
| 10536 | FPCQNGPHN |
| 10537 | FPDRMDYVE |
| 10538 | FPGGQKPCL |
| 10539 | FPHFTCWCD |
| 10540 | FPHGYAMIW |
| 10541 | FPIQGNHFP |
| 10542 | FPKAPKYYL |
| 10543 | FPKNNLTKC |
| 10544 | FPLCVDPGP |
| 10545 | FPNIDNPKA |
| 10546 | FPPPIHLAI |
| 10547 | FPPQQEFIH |
| 10548 | FPQWGKEPI |
| 10549 | FPRTTNTRV |
| 10550 | FPSHDDFLG |
| 10551 | FPVTCGTVF |
| 10552 | FPWYSLILT |
| 10553 | FPYMAEQYR |
| 10554 | FPYWDIDDT |
| 10555 | GPCMLYEMC |
| 10556 | GPDPWSQWW |
| 10557 | GPDTEYRGC |
| 10558 | GPFADPPAT |
| 10559 | GPFLSVPAH |
| 10560 | GPFRFEWEW |
| 10561 | GPGTLRHLL |
| 10562 | GPHWYWHDM |
| 10563 | GPINPYAHG |
| 10564 | GPLSKHFDV |
| 10565 | GPMHNTGPP |
| 10566 | GPPPSDLRS |
| 10567 | GPPWKHCDA |
| 10568 | GPSCYLDYW |
| 10569 | GPTIWIHTY |
| 10570 | GPTIYQYMQ |
| 10571 | GPTKLNYNN |
| 10572 | GPVVPCNSW |
| 10573 | GPVWEFEWM |
| 10574 | GPWHACKIN |
| 10575 | HPCCDRYLA |
| 10576 | HPDAPGPIQ |
| 10577 | HPDVRNYEN |
| 10578 | HPEVDSSGA |
| 10579 | HPGCEYSWE |
| 10580 | HPGIEVDRG |
| 10581 | HPGWTHAVF |
| 10582 | HPHDVFHET |
| 10583 | HPILPAQYF |
| 10584 | HPISPWQWL |
| 10585 | HPLSRGSES |
| 10586 | HPMFEAYQN |
| 10587 | HPNFIWCNH |
| 10588 | HPNGNEREH |
| 10589 | HPNPWGVFW |
| 10590 | HPRFNRLQV |
| 10591 | HPSFIKCQV |
| 10592 | HPYLCTIRI |
| 10593 | IPDAWWANM |
| 10594 | IPHRNIHPS |
| 10595 | IPLWETMNM |
| 10596 | IPNDRDRCA |
| 10597 | IPRVHWKAE |
| 10598 | IPTFRTMKI |
| 10599 | IPWFDFAME |
| 10600 | KPAPWMRME |
| 10601 | KPCPPHAYV |
| 10602 | KPEAWAVCG |
| 10603 | KPESKMLSE |
| 10604 | KPFVSECTE |
| 10605 | KPGFRYFAE |
| 10606 | KPGTVKDCD |
| 10607 | KPILMSEEA |
| 10608 | KPITEGRRD |
| 10609 | KPKCCQYNW |
| 10610 | KPMCDGVAY |
| 10611 | KPMCLYVLT |
| 10612 | KPNDCLREG |
| 10613 | KPPMPTNTG |
| 10614 | KPPYSCREQ |
| 10615 | KPQHENKQL |
| 10616 | KPSMYPRNS |
| 10617 | KPTDMVGAW |
| 10618 | KPTQIGGEH |
| 10619 | KPVEPTTNP |
| 10620 | LPAAYFWGA |
| 10621 | LPAWYRARC |
| 10622 | LPIEQQKSW |
| 10623 | LPIESEGEI |
| 10624 | LPLGQADEA |
| 10625 | LPLVYAKNW |
| 10626 | LPLYFHEHY |
| 10627 | LPMDEKEGA |
| 10628 | LPMQCDETY |
| 10629 | LPMVWGNPK |
| 10630 | LPMYIEHMC |
| 10631 | LPNPVNATQ |
| 10632 | LPNWFCRNY |
| 10633 | LPPAHMFYC |
| 10634 | LPQHYGIWH |
| 10635 | LPQPYPDNY |
| 10636 | LPQRGTPIV |
| 10637 | LPRHIYKST |
| 10638 | LPSHELWTW |
| 10639 | LPTSDCRAE |
| 10640 | LPTYNKYVQ |
| 10641 | MPAHANSTC |
| 10642 | MPCTCSYPH |
| 10643 | MPDNFGNGP |
| 10644 | MPIMMNVSP |
| 10645 | MPMDLNYIQ |
| 10646 | MPMFCQIEQ |
| 10647 | MPPFHENES |
| 10648 | MPPPVQDWN |
| 10649 | MPPVRLYSY |
| 10650 | MPQDCGWNL |
| 10651 | MPRDGTWWC |
| 10652 | MPSHGYWYQ |
| 10653 | MPSPYGEAY |
| 10654 | MPSQGVIWH |
| 10655 | MPSVWGPDM |
| 10656 | MPTEYEAHG |
| 10657 | MPTQAYMPQ |
| 10658 | MPVDIANQM |
| 10659 | MPWWYGCIC |
| 10660 | NPCFMIWYT |
| 10661 | NPCMLQDHH |
| 10662 | NPCTFWNNE |
| 10663 | NPGHAWATM |
| 10664 | NPGWWCRMW |
| 10665 | NPIMYPCLS |
| 10666 | NPLIRDMWT |
| 10667 | NPLKLDYPE |
| 10668 | NPPWLQPFS |
| 10669 | NPQFLHFQI |
| 10670 | NPRAMDAAH |
| 10671 | NPRHDAGHS |
| 10672 | NPSWNACNV |
| 10673 | NPTMPGIRG |
| 10674 | NPTQDFKVM |
| 10675 | NPVTGEYSL |
| 10676 | NPWHYTYAF |
| 10677 | NPYLHMFNQ |
| 10678 | NPYNQHCIC |
| 10679 | NPYRTFTDG |
| 10680 | QPAFEHAQV |
| 10681 | QPAHMEVMY |
| 10682 | QPAHPGTGL |
| 10683 | QPAQYAVMH |
| 10684 | QPCDITYQS |
| 10685 | QPDCEWPHN |
| 10686 | QPDMYDCYA |
| 10687 | QPEFTYLQF |
| 10688 | QPHSSVTLD |
| 10689 | QPPAAAKQD |
| 10690 | QPTDDVIQG |
| 10691 | QPTQRMYHC |
| 10692 | QPVTFYEKH |
| 10693 | QPVWFPKCW |
| 10694 | QPWMVWNSH |
| 10695 | QPWMYVMGS |
| 10696 | QPWTFWWNR |
| 10697 | QPYCERTGI |
| 10698 | RPAVDDEEC |
| 10699 | RPCPNMTDG |
| 10700 | RPDVRNELC |
| 10701 | RPELGGQQT |
| 10702 | RPETTAERP |
| 10703 | RPHHFWVPF |
| 10704 | RPKECEQMW |
| 10705 | RPLDTSQED |
| 10706 | RPMFGAVAG |
| 10707 | RPMHGDYDD |
| 10708 | RPNCVMYDK |
| 10709 | RPPDEIWGH |
| 10710 | RPRDNGVEG |
| 10711 | RPWSSEFRV |
| 10712 | SPAEIPSIF |
| 10713 | SPCPNGVRQ |
| 10714 | SPDVGWTES |
| 10715 | SPEVTEYGF |
| 10716 | SPFMWWRWW |
| 10717 | SPGFILGGY |
| 10718 | SPGKFWHPC |
| 10719 | SPHFQKHPS |
| 10720 | SPIRIDWQG |
| 10721 | SPISDLRNC |
| 10722 | SPIWEGERT |
| 10723 | SPKKAEQAI |
| 10724 | SPLHVHWQS |
| 10725 | SPLISVLEG |
| 10726 | SPLQNYEER |
| 10727 | SPMDTTEPK |
| 10728 | SPMQKQKVS |
| 10729 | SPQTCSFPT |
| 10730 | SPRCMTNGG |
| 10731 | SPSGIMEGN |
| 10732 | SPYQCPQAS |
| 10733 | TPDHKTRAH |
| 10734 | TPDPDLNNR |
| 10735 | TPGSDMSMV |
| 10736 | TPHSSMKVG |
| 10737 | TPKLEFPVD |
| 10738 | TPLLTSGHE |
| 10739 | TPLMAYWIP |
| 10740 | TPMDYEDQE |
| 10741 | TPMFDRHSL |
| 10742 | TPMLGMLWW |
| 10743 | TPMWQASWQ |
| 10744 | TPNCMGQRM |
| 10745 | TPNVWEQQM |
| 10746 | TPPHKCSVG |
| 10747 | TPQCAMWNE |
| 10748 | TPSQVASDD |
| 10749 | TPSRNMVDL |
| 10750 | TPYIRHHFE |
| 10751 | TPYTWCWPD |
| 10752 | VPCPKWSMI |
| 10753 | VPEQGRWEA |
| 10754 | VPKLLDTDQ |
| 10755 | VPLLILNNN |
| 10756 | VPLVSYSQQ |
| 10757 | VPMMCQWSQ |
| 10758 | VPQHKNEFQ |
| 10759 | VPQWIDERW |
| 10760 | VPRHWAGVR |
| 10761 | VPRYNSDEQ |
| 10762 | VPSCQCSES |
| 10763 | VPTIKVTRA |
| 10764 | VPTQSVKGC |
| 10765 | VPTSYWCTN |
| 10766 | VPWFVMLMM |
| 10767 | VPWMVLDNN |
| 10768 | WPALFVADA |
| 10769 | WPDPFIHAT |
| 10770 | WPEATHRFA |
| 10771 | WPKYDMPLH |
| 10772 | WPMCPVCYF |
| 10773 | WPMTRTNGH |
| 10774 | WPNYHSWYE |
| 10775 | WPPNGKWFM |
| 10776 | WPQRGTMDM |
| 10777 | WPTGHCAYW |
| 10778 | WPYYCCIEH |
| 10779 | YPANKCEWE |
| 10780 | YPASYDCHP |
| 10781 | YPDTVQKSP |
| 10782 | YPDWWEWHT |
| 10783 | YPEGEMNYE |
| 10784 | YPFQPIEEP |
| 10785 | YPFWAGWGD |
| 10786 | YPGMKICGH |
| 10787 | YPIIMLPCR |
| 10788 | YPMQGGIRW |
| 10789 | YPNDAKHGN |
| 10790 | YPNLHHSMM |
| 10791 | YPSDGLFEQ |
| 10792 | YPSGHKPYW |
| 10793 | YPSINCPTN |
| 10794 | YPSPPERLE |
| 10795 | YPVNCTQKW |
| 10796 | YPWENNAHL |
| 10797 | YPWMTACHD |
| 10798 | YPYICEFVW |
| 10799 | YPYPRLWEC |
| 10800 | NEPYCIWAY |
| 10801 | GRPVMHRHS |
| 10802 | DSPSNHPAV |
| 10803 | YYPFPGPTD |
| 10804 | AAPIPGKAF |
| 10805 | AFPNVMYQN |
| 10806 | AGPKMIEPW |
| 10807 | AHPQQPHVT |
| 10808 | AIPMIGSYC |
| 10809 | AIPQEGVCC |
| 10810 | AKPYKFSGE |
| 10811 | AMPIVMWHT |
| 10812 | ANPADWHWY |
| 10813 | ANPFVLAPW |
| 10814 | ARPWWMLEQ |
| 10815 | ASPPDYKAQ |
| 10816 | AVPQMTGWC |
| 10817 | AYPHCEAVM |
| 10818 | AYPSGIRAH |
| 10819 | CCPVDNEFW |
| 10820 | CDPPIDCYI |
| 10821 | CDPSYELNI |
| 10822 | CDPWHKYNG |
| 10823 | CGPFRHQNG |
| 10824 | CHPQEWHGQ |
| 10825 | CIPDFISEY |
| 10826 | CLPWVMSIA |
| 10827 | CMPPDCKCP |
| 10828 | CNPGWPKPG |
| 10829 | CTPYPPRDQ |
| 10830 | CVPNSHIWQ |
| 10831 | CYPTLEKHA |
| 10832 | DFPLNPVQT |
| 10833 | DFPLQVTGY |
| 10834 | DFPRNNAIM |
| 10835 | DGPGTQCIK |
| 10836 | DLPAGQRMF |
| 10837 | DLPLPKLMQ |
| 10838 | DNPGYLKEE |
| 10839 | DSPNYVWTH |
| 10840 | DTPEYAPSM |
| 10841 | DTPLQYKAQ |
| 10842 | DYPKGEKCD |
| 10843 | EEPELQRLN |
| 10844 | EHPPHAFAH |
| 10845 | EKPLYYHHA |
| 10846 | ELPATLFQF |
| 10847 | ENPQFVRSI |
| 10848 | ESPCHQNQG |
| 10849 | EYPSYWSDG |
| 10850 | EYPYDVPNF |
| 10851 | FAPEICGCC |
| 10852 | FDPVCKCMK |
| 10853 | FEPQNPTFQ |
| 10854 | FFPTMHARF |
| 10855 | FIPCVISNQ |
| 10856 | FLPFWKAFH |
| 10857 | FLPTSKLHS |
| 10858 | FLPTYDGMH |
| 10859 | FNPPIRQLW |
| 10860 | FQPFTSNLL |
| 10861 | FQPYMTSTS |
| 10862 | FRPIISMKR |
| 10863 | FRPMITCWW |
| 10864 | FSPPMVMKY |
| 10865 | FSPYFMVPH |
| 10866 | FTPDSLIVH |
| 10867 | FTPECTRGM |
| 10868 | FVPHDCNRD |
| 10869 | GAPFDELPH |
| 10870 | GCPLIVPDL |
| 10871 | GDPLVIWLC |
| 10872 | GFPREMYTE |
| 10873 | GGPEKEAHR |
| 10874 | GGPPIDEGN |
| 10875 | GHPFKDYHS |
| 10876 | GMPWEDISN |
| 10877 | GNPCSPKIN |
| 10878 | GNPKLLESF |
| 10879 | GNPPTWLCP |
| 10880 | GNPYMYPWH |
| 10881 | GRPWDPYFE |
| 10882 | GTPQWWNKG |
| 10883 | GTPSMHMGI |
| 10884 | GVPQDRIQE |
| 10885 | HCPNQGRRS |
| 10886 | HFPRSEAHR |
| 10887 | HGPMEVMHE |
| 10888 | HHPFAKPII |
| 10889 | HIPQHSRMC |
| 10890 | HKPEQMEAY |
| 10891 | HKPKMWMYG |
| 10892 | HNPLIRTMV |
| 10893 | HNPLQENDC |
| 10894 | HQPELMTDN |
| 10895 | HRPDWNDGK |
| 10896 | HRPPCEMGD |
| 10897 | HYPAYQKAC |
| 10898 | HYPWTPWTP |
| 10899 | ICPTKGMEA |
| 10900 | ICPVCSWDC |
| 10901 | IDPEIYAWC |
| 10902 | IDPLDGNHF |
| 10903 | IHPQQWWDH |
| 10904 | IIPHQNIMW |
| 10905 | IKPEPMWMT |
| 10906 | ILPPEKHQA |
| 10907 | IMPNNLEMM |
| 10908 | INPRKLSSK |
| 10909 | IQPMYVIYQ |
| 10910 | IQPWDYVRP |
| 10911 | ISPLNICWF |
| 10912 | ITPEVLKTT |
| 10913 | KCPLGKPAS |
| 10914 | KCPRGGMNC |
| 10915 | KDPCCGCHN |
| 10916 | KGPPHKDDQ |
| 10917 | KHPNIWCAN |
| 10918 | KLPDTYKNF |
| 10919 | KMPPPAKDI |
| 10920 | KTPHCIKHY |
| 10921 | KVPVLTAGG |
| 10922 | KWPTFKWPK |
| 10923 | KYPEPGRGT |
| 10924 | LAPQLGWRN |
| 10925 | LCPGEWLVI |
| 10926 | LEPKDLAAP |
| 10927 | LEPPSTVNV |
| 10928 | LFPNQCNFV |
| 10929 | LFPQHIAQP |
| 10930 | LGPHRWYGH |
| 10931 | LHPWMPAGR |
| 10932 | LKPSWQDKA |
| 10933 | LLPMDEMKT |
| 10934 | LMPCHGDIA |
| 10935 | LMPELGCTD |
| 10936 | LSPLIAGIY |
| 10937 | LTPDVRINA |
| 10938 | LTPSCRHKW |
| 10939 | LVPTLINCL |
| 10940 | LWPSSCQMK |
| 10941 | MAPQPMGGG |
| 10942 | MCPKTFRFE |
| 10943 | MEPKKMKVW |
| 10944 | MEPMSRDHL |
| 10945 | MEPWEQCHI |
| 10946 | MGPRYDKEW |
| 10947 | MHPDSYNVT |
| 10948 | MKPDQNHHK |
| 10949 | MLPLPNEFQ |
| 10950 | MLPMAWYQI |
| 10951 | MLPMRQLTQ |
| 10952 | MLPWSSRSN |
| 10953 | MMPDILRSY |
| 10954 | MQPCWQPQC |
| 10955 | MRPQQVWNG |
| 10956 | MSPQPELSG |
| 10957 | MVPAAGPGE |
| 10958 | MVPKMMRSQ |
| 10959 | MWPAMKVCW |
| 10960 | NCPQPNCGW |
| 10961 | NMPERKLTF |
| 10962 | NMPIGGYCC |
| 10963 | NNPQKQQSR |
| 10964 | NNPSNSLIW |
| 10965 | NTPEFHWQQ |
| 10966 | NTPLDFTGE |
| 10967 | NTPNNMEAR |
| 10968 | NTPQQGQKG |
| 10969 | NVPTEPMDH |
| 10970 | NWPVSDEDM |
| 10971 | NYPCTDKNH |
| 10972 | NYPVGYHEL |
| 10973 | QAPMVQDTQ |
| 10974 | QAPWKHEGP |
| 10975 | QCPDEKAEF |
| 10976 | QCPIQSVVC |
| 10977 | QDPFWRGLH |
| 10978 | QHPDQGQHC |
| 10979 | QIPYEMWWS |
| 10980 | QLPCIKKCP |
| 10981 | QLPGCRTDQ |
| 10982 | QLPKMWLPN |
| 10983 | QMPNGEIVT |
| 10984 | QNPDDVINL |
| 10985 | QNPSPMMHR |
| 10986 | QNPSTYCHY |
| 10987 | QQPWPHQRG |
| 10988 | QRPTMLQGY |
| 10989 | QSPPIFPTT |
| 10990 | QSPWKLWSH |
| 10991 | QWPDFMVTI |
| 10992 | QYPQLPHWA |
| 10993 | REPFLHVNS |
| 10994 | RFPVKLGAM |
| 10995 | RHPQLGLPH |
| 10996 | RIPWAELMK |
| 10997 | RKPWREHNI |
| 10998 | RLPYYVQFC |
| 10999 | RNPCSKHEV |
| 11000 | RNPRDYPMF |
| 11001 | RQPANRFKE |
| 11002 | RTPFPHDSL |
| 11003 | RWPSEKEKF |
| 11004 | SAPPSFAQK |
| 11005 | SAPVEWMRK |
| 11006 | SCPNRESGR |
| 11007 | SGPRTDCPL |
| 11008 | SGPVVMIQS |
| 11009 | SMPHACYNG |
| 11010 | SMPTTSSSC |
| 11011 | SSPDNWKHM |
| 11012 | STPLGNSAP |
| 11013 | STPMVCEMG |
| 11014 | SVPHEVHCE |
| 11015 | SVPLKFWTL |
| 11016 | SWPDEPSVA |
| 11017 | SWPHCEIEI |
| 11018 | SYPLTRWPA |
| 11019 | SYPYMHMQD |
| 11020 | TAPQCVSDA |
| 11021 | TCPNLTAYC |
| 11022 | TCPRNLCNY |
| 11023 | TDPLCKHQT |
| 11024 | TDPLNPLCD |
| 11025 | TEPPCPGQT |
| 11026 | TEPSIMNEV |
| 11027 | TFPCNRFGI |
| 11028 | TFPSIQWSR |
| 11029 | TGPNFQPAL |
| 11030 | TIPFCKKCE |
| 11031 | TIPYEANCA |
| 11032 | TKPQITEIS |
| 11033 | TQPCMPNRT |
| 11034 | TRPPSSLDE |
| 11035 | TTPTWQNSP |
| 11036 | TVPHFYFYG |
| 11037 | TWPMSCDCD |
| 11038 | TYPFGWDKH |
| 11039 | VAPAILYRC |
| 11040 | VAPIHDHRI |
| 11041 | VDPAMMPDN |
| 11042 | VFPTASQLC |
| 11043 | VHPEQTLIG |
| 11044 | VHPFHKYEQ |
| 11045 | VIPVHNMCG |
| 11046 | VKPYNGLVQ |
| 11047 | VLPHFWNQG |
| 11048 | VLPKSHFFP |
| 11049 | VMPEESVRN |
| 11050 | VMPTNILEE |
| 11051 | VMPYAQGIS |
| 11052 | VQPCFMMDH |
| 11053 | VQPFPYCCE |
| 11054 | VQPHPFNPH |
| 11055 | VQPPILEQH |
| 11056 | VQPWWDAMG |
| 11057 | VRPLHNFID |
| 11058 | VSPANADND |
| 11059 | VTPNLQVQS |
| 11060 | VTPYDWKFQ |
| 11061 | WAPSPRQDR |
| 11062 | WDPVYGERN |
| 11063 | WEPDGIVPQ |
| 11064 | WHPKCYDVF |
| 11065 | WHPMDKNFR |
| 11066 | WIPTEIYGH |
| 11067 | WKPEGQNFG |
| 11068 | WLPRMPSCD |
| 11069 | WMPCCPHGC |
| 11070 | WMPGMHWRC |
| 11071 | WMPSTTRWN |
| 11072 | WQPDWMYEH |
| 11073 | WRPPVRVQD |
| 11074 | WSPERERFE |
| 11075 | WWPIWWIPQ |
| 11076 | WYPLDVLAS |
| 11077 | WYPMLWELE |
| 11078 | LSICQPVMP |
| 11079 | TTRCFRHAK |
| 11080 | WATCDAPPV |
| 11081 | NYVCSTSGY |
| 11082 | LTKCMNKGW |
| 11083 | SCECPCCLA |
| 11084 | VCECCRQNI |
| 11085 | ALCCFCCQG |
| 11086 | TSYCIQTVD |
| 11087 | DQECCQTLF |
| 11088 | QTQVPIWNT |
| 11089 | LATHPPYTY |
| 11090 | LTYNPTMIG |
| 11091 | TGIIPIRVC |
| 11092 | TCQEPHNIT |
| 11093 | QVYAPEIEG |
| 11094 | DYQSERLWS |
| 11095 | KEQEQRPVR |
| 11096 | ISMQNRCAD |
| 11097 | IFQLSRPAW |
| 11098 | ECKTHRDVY |
| 11099 | YHNQDRHYL |
| 11100 | KVDTVRNPK |
| 11101 | TVSRGRPVA |
| 11102 | NQAYGRGHV |
| 11103 | SVRLNEPHV |
| 11104 | CLVNDMPRK |
| 11105 | AHTFEAPKW |
| 11106 | IWNYCVPKH |
| 11107 | HWEQHNPIW |
| 11108 | CIDWAEMPQ |
| 11109 | ACVMYDCPP |
| 11110 | WVGNANRPA |
| 11111 | SVEVGCSPG |
| 11112 | CVFSTNHPA |
| 11113 | AMRQIIAPC |
| 11114 | TVVFSENEI |
| 11115 | WKGRWTTTI |
| 11116 | NSHYTEGVI |
| 11117 | AWTPTEKQI |

### Example 15

AAV5 Variants with Tissue Tropism in Thyroid Gland

This example describes engineered AAV5 variants with tissue tropism in thyroid gland that were discovered using the methods and systems described in EXAMPLE 1-EXAMPLE 2.

CNS, liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive thyroid gland tropism. The results are shown in FIG. 18L which shows the positional frequency of each amino acid in the 581 to 589 region of AAV5 VP1 in thyroid gland tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in thyroid gland over the frequency of that given amino acid residue occurring at the specified position in variants identified in all other harvested tissues was analyzed to identify a set of sequence rules for capsids that preferentially target thyroid gland tissue. With reference to TABLE 29 below, and SEQ ID NO: 2 (AAV5 VP1), the following amino acids can, thus, be independently mutated, in any combination, at any one or more positions Xaa1-Xaa9, to provide a VP1 capsid with enhanced thyroid gland tropism as compared wildtype AAV5 VP1 capsid (SEQ ID NO: 1), where thyroid gland tropism here refers to properties that are preferred for thyroid gland transduction over properties that are preferred for transduction of all other harvested tissues.

TABLE 29
Thyroid Gland Tropism Rules

Xaa1 is selected from A, K, M, N, Q, or R
Xaa1 is selected from K, N or Q
Xaa1 is K
Xaa2 is selected from A, F, K, L, M, T, V, or W
Xaa2 is selected from F, V, or W
Xaa2 is W
Xaa3 is selected from A, I, K, R, S, T, V, or W
Xaa3 is selected from A, R or T
Xaa3 is R
Xaa4 is selected from A, D, E, I, P, or V
Xaa4 is selected from A, E, or I
Xaa4 is A
Xaa5 is selected from F, I, M, Q, V, or Y
Xaa5 is M, V, Y
Xaa5 is M
Xaa6 is selected from H, M, N, or Y
Xaa6 is N
Xaa7 is selected from H, I, N, Q, S, or W
Xaa7 is selected from H, I, or N
Xaa7 is H
Xaa8 is selected from A, D, F, Q, S, or Y
Xaa8 is selected from A, F, or S
Xaa8 is F
Xaa9 is selected from A, Q, S, or Y
Xaa9 is selected from A or S
Xaa9 is A

TABLE 30 below provides sequences of the 581 to 589 region for variant AAV VP1 capsid polypeptides that were found in thyroid gland tissue and comport to one or more of the rules provided in TABLE 29. The present disclosure, thus, provides for rAAVs composed of engineered AAV5 VP1 capsid polypeptides having a SEQ ID NO: 2, wherein the Xaa1 to Xaa9 region is any one of SEQ ID NO: 40438-SEQ ID NO: 41437, as disclosed in TABLE 30. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 30
Sequences of the 581 to 589 Region in AAV5
VP1 Capsid Polypeptide that Drive Thyroid
Gland Tissue Tropism

| SEQ | |
|---|---|
| ID | 581-589 |
| NO | Sequence |
| 40438 | KAEDSQPDC |
| 40439 | KAMQVLENC |
| 40440 | KASWIPSNK |
| 40441 | KCEDNTFAS |
| 40442 | KCTDLGEYF |
| 40443 | KDHEEHGPK |
| 40444 | KERTAWSRE |
| 40445 | KFMQDPCNS |
| 40446 | KGELMCLDV |
| 40447 | KGPKECAAT |
| 40448 | KHILWGSLA |
| 40449 | KIGISEQDF |
| 40450 | KKYTDQDYG |
| 40451 | KMTPVEADA |
| 40452 | KNPTVFWKM |
| 40453 | KQAIYLDLD |
| 40454 | KSAMQEGVA |
| 40455 | KSFGSEVFF |
| 40456 | KSKWGHYWD |
| 40457 | KSMDLGQFM |
| 40458 | KSNQTYCAA |
| 40459 | KSYCLHMKQ |
| 40460 | KTGMACDIN |
| 40461 | KTLVKQMDY |
| 40462 | KTVWVYRVH |
| 40463 | KTYPHCFPV |
| 40464 | KVEDGLQWH |
| 40465 | KVEGMVTQS |
| 40466 | KVIEFCGEE |
| 40467 | KWLEANMTG |
| 40468 | KYAFRYEAS |
| 40469 | KCLQMCPNG |
| 40470 | KAIGWFHFD |
| 40471 | KAYTVEMDP |
| 40472 | KCCLPWQNI |
| 40473 | KCEQWQKVC |
| 40474 | KCHVGGNVY |
| 40475 | KCNPWSEPT |
| 40476 | KDDGDDNIP |
| 40477 | KDRDQVHWM |
| 40478 | KEGNMDACS |
| 40479 | KEYDPQHWW |
| 40480 | KFIYGQREV |
| 40481 | KFKQEDAQM |
| 40482 | KGIWQDHSC |
| 40483 | KHCENCEYF |
| 40484 | KIAPVAASP |
| 40485 | KIEMLCCQG |
| 40486 | KILNPNDSY |
| 40487 | KKRRDTQIY |
| 40488 | KLHIWNVPC |
| 40489 | KMQPNGEWP |
| 40490 | KNDQTTYPE |
| 40491 | KNEPDNLCE |
| 40492 | KPCTFAPFM |
| 40493 | KPEWAMMYD |
| 40494 | KPYQDIARM |
| 40495 | KQEIQGVCR |
| 40496 | KQETRTTDH |
| 40497 | KQFWMKVEV |
| 40498 | KQMLQQCFG |
| 40499 | KQWELYQID |
| 40500 | KRVVKFTST |
| 40501 | KRYTHGKNW |
| 40502 | KSADMPTTL |
| 40503 | KSFLHPAAH |
| 40504 | KSFYMCRCG |
| 40505 | KSYVGSYES |
| 40506 | KTCELTAMR |
| 40507 | KTEMSDMHW |
| 40508 | KTLVCVNWF |
| 40509 | KTYLWEHES |
| 40510 | KVSEAHYDL |
| 40511 | KVTTETGVP |
| 40512 | KVVAVNQSL |
| 40513 | KWQQAEYDE |
| 40514 | KYCHWWYKL |
| 40515 | KYQNAKPVA |
| 40516 | KKNSMDCQV |
| 40517 | KPYFKDCPR |
| 40518 | KSIFQATLQ |
| 40519 | KTAHDKDID |
| 40520 | KHSSWCCYT |
| 40521 | KSEHHWINA |
| 40522 | KVGMDQTAV |
| 40523 | KFFYTELMH |
| 40524 | KDFNFEAQG |
| 40525 | KDQQAERHV |
| 40526 | KDYANFGMN |
| 40527 | KHIHCTIQC |
| 40528 | KNPCLVWQK |
| 40529 | KSYEMVNQW |
| 40530 | KTGWDSHWS |
| 40531 | KVAYMWYNW |
| 40532 | KHGVLPMPA |
| 40533 | KTLHMLESK |
| 40534 | KCGLTPGVE |
| 40535 | KVDTVRNPK |
| 40536 | KAHDITWNL |
| 40537 | KAEMKMGCN |
| 40538 | KTTIVPAWN |
| 40539 | KAIPQGSSC |
| 40540 | KMTNSNENS |
| 40541 | KWYQFGMVA |
| 40542 | KFDVIRQAM |
| 40543 | KQLTFPMLA |
| 40544 | KQEWHVHTE |
| 40545 | KIDQTQWQR |
| 40546 | KRFVDMPEM |
| 40547 | KLYGLNHSC |
| 40548 | KIQPMMVKA |
| 40549 | KMLHKVWID |
| 40550 | KGPIFHSAY |
| 40551 | KGMQMWNC |
| | E |
| 40552 | IWKFKNAHL |
| 40553 | IWNDQWDCP |
| 40554 | IWNWMTEDC |
| 40555 | IWTLNWAQL |
| 40556 | LWEQWSTLS |
| 40557 | LWQFCSICE |
| 40558 | LWVECHCNQ |
| 40559 | MWVELPSDD |
| 40560 | NWIHWNACF |
| 40561 | NWMERDHNH |
| 40562 | NWSYCNVSA |
| 40563 | NWTHQDMFD |
| 40564 | NWYFMSQTD |
| 40565 | QWAKWDWSS |
| 40566 | QWHQFNRMH |
| 40567 | QWTTITKEE |
| 40568 | RWKSQEEIQ |
| 40569 | SWAFANHTH |
| 40570 | SWEQNVVST |
| 40571 | TWTAMEIYG |
| 40572 | SWSPTGVAN |
| 40573 | AWDHTMPYG |
| 40574 | AWGNRYHYC |
| 40575 | AWHDYFRDN |
| 40576 | AWMDCSIMC |
| 40577 | CWQAALHSA |
| 40578 | CWWCALLST |
| 40579 | DWIMDTPKP |
| 40580 | DWIVDTPKP |
| 40581 | DWNYYACYT |
| 40582 | EWICVQSHN |
| 40583 | GWMIAMDGK |
| 40584 | GWNTFIWGQ |
| 40585 | GWTLNPSEG |
| 40586 | HWIPQWARS |
| 40587 | HWISFERHQ |
| 40588 | IWDFKCTQC |
| 40589 | IWKDYLHVG |
| 40590 | LWCNENAMD |
| 40591 | LWQQIPWGK |
| 40592 | MWAAMPVG |
| | H |
| 40593 | MWEQEPNRN |
| 40594 | MWFAENNGL |
| 40595 | MWGINGVAY |
| 40596 | MWRPARYKS |
| 40597 | NWEDSPWCY |
| 40598 | NWEPCWWA |
| | H |
| 40599 | NWGPQQSER |
| 40600 | NWNKTRFTC |
| 40601 | NWVSHDKEA |
| 40602 | PWAETEKPN |
| 40603 | PWIPNQIKQ |
| 40604 | PWTCNENQW |
| 40605 | QWPEMTCCD |
| 40606 | QWTRTGSSF |
| 40607 | RWHNGENCS |
| 40608 | RWNARWELG |
| 40609 | RWRCRMQGN |
| 40610 | SWIDMAMM |
| | Q |
| 40611 | SWTALGSVP |
| 40612 | SWTLRMWAS |
| 40613 | TWLPVGAAH |
| 40614 | TWVNFPTHC |
| 40615 | VWWFAPMEA |
| 40616 | WWAQPWHA |
| | L |
| 40617 | WWTDMSSW |
| | T |
| 40618 | YWWLDHANF |
| 40619 | GWTTNLHYH |
| 40620 | NWVVWPME |
| | N |
| 40621 | RWVCANAQD |
| 40622 | CWECYQFKR |
| 40623 | CWSAIMTGC |
| 40624 | DWMQGGIYN |
| 40625 | EWFEDEVLW |
| 40626 | EWGMHTSNR |
| 40627 | FWSGYNAEK |
| 40628 | GWTHFHWQE |
| 40629 | LWKTILEGV |
| 40630 | RWGMYDSYY |
| 40631 | RWTDTNFRG |
| 40632 | TWAMMGQW |
| | W |
| 40633 | LWKLGNKVF |
| 40634 | TWTSRCELL |
| 40635 | VWNPDLKMN |
| 40636 | EWTKKTADH |
| 40637 | IWNYCVPKH |
| 40638 | YWAFPCCGF |
| 40639 | CWGPIMHPF |
| 40640 | TWKSPRGYM |
| 40641 | AWMCCARHD |
| 40642 | CWWNFGKDC |
| 40643 | SWCAMIYEM |
| 40644 | IVRFYFKSW |
| 40645 | LIRQGDCFI |
| 40646 | LTRDRFTEW |
| 40647 | LYRTSIALV |
| 40648 | MERYGHCHP |
| 40649 | MLRAESGLT |
| 40650 | NNRTYDWWN |
| 40651 | PVRMFTESG |
| 40652 | QGRQILKAI |
| 40653 | QHRYYTCQS |
| 40654 | QLRKHYTLL |
| 40655 | QYRPTWVHV |
| 40656 | RFRWVHFHW |
| 40657 | SGRCNALNT |
| 40658 | TIRDSVFGC |
| 40659 | VAREDEWQN |
| 40660 | VCRTQESSA |
| 40661 | VHRHRAHKF |
| 40662 | VPRWVERAH |
| 40663 | VVRSYNFGE |
| 40664 | NDRHVPCWD |
| 40665 | AFRNKEFYV |
| 40666 | AHRDYACTN |
| 40667 | CMRGDKRNR |
| 40668 | CMRMDGNDR |
| 40669 | CNRPKCPPM |
| 40670 | DARFGLNFG |
| 40671 | DARFYITEG |
| 40672 | DHRETDAEE |
| 40673 | DYRNYFIWP |
| 40674 | EARCTDMCY |
| 40675 | EFRPVLECC |
| 40676 | EIRYYYPES |
| 40677 | ELRWDARPH |
| 40678 | EMRITNARC |
| 40679 | ERRMEQAQT |
| 40680 | ESRAGYMHY |
| 40681 | EYRCVYRKC |
| 40682 | EYRLCPQTS |
| 40683 | FHREQAAHQ |
| 40684 | HSRQSEETI |
| 40685 | IAREPLMAN |
| 40686 | IDRKDRRES |
| 40687 | IIRNRCAEP |
| 40688 | IYRLMHWDN |
| 40689 | LMRSTVKNG |
| 40690 | LPRCVIAAC |
| 40691 | LTREDFTQM |
| 40692 | MLRAMDIDA |
| 40693 | MVRDKNNDH |
| 40694 | NRRMVVAQS |
| 40695 | NTREYPEYI |
| 40696 | PMRMAIEFD |
| 40697 | PSRPCTYHS |
| 40698 | QGRWMYHW |
| | W |
| 40699 | QLREIQMIQ |
| 40700 | SNRCLTYEF |
| 40701 | SQRDEMFAL |
| 40702 | SQRHIECIG |
| 40703 | STRDWMSCM |
| 40704 | TDRMLAGQR |
| 40705 | TFRMSVTGV |
| 40706 | TIRSFNLEA |
| 40707 | TMRTHSSWW |
| 40708 | TYRVFMVHD |
| 40709 | VFRMVMEQY |
| 40710 | VYRLREKQE |
| 40711 | IGRHQMTDK |
| 40712 | ANRADWQHL |
| 40713 | QCRHTDQVK |
| 40714 | ETRGFVKCH |
| 40715 | ETRVREDAV |
| 40716 | QDRRPENRF |
| 40717 | CERSKCPKS |
| 40718 | DIRSYKGFQ |
| 40719 | QVRDIANMC |
| 40720 | VKRLENEWH |
| 40721 | YGRLREAMH |
| 40722 | DDRVKMFHY |
| 40723 | ANRMIWYPY |
| 40724 | GHRPKLCDQ |
| 40725 | VNRGDLSLF |
| 40726 | QSRCDPVSP |
| 40727 | EHRDNTTRP |
| 40728 | GVRNALNLS |
| 40729 | GIRCISKAG |
| 40730 | LTRSGPIGQ |
| 40731 | MTRVSMTWR |
| 40732 | CCRQIWVQY |
| 40733 | STRPIACHS |
| 40734 | NNRALMSVE |
| 40735 | TVRHQMDAY |
| 40736 | NNRRQFGYV |
| 40737 | SSRMQMQGP |
| 40738 | MFEAIVTGL |
| 40739 | MHGAFVDTQ |
| 40740 | MNVAYHGFS |
| 40741 | MTQADPCIG |
| 40742 | NAYAAGNIG |
| 40743 | NHSANPHGY |
| 40744 | NIMAIHHDS |
| 40745 | NMVAMNEPR |
| 40746 | NVWAWKEVE |
| 40747 | QLIAIHDIN |
| 40748 | QTVAHENCD |
| 40749 | QTVAPGGHC |
| 40750 | RADATGVPN |
| 40751 | REDAYLAGH |
| 40752 | RSEAGIRQI |
| 40753 | SGLAFDEDD |
| 40754 | SHVAYKLLD |
| 40755 | TMVAFSSCL |
| 40756 | TSYAMWSDV |
| 40757 | VASAMRHHT |
| 40758 | VTEAFPTSV |
| 40759 | VVDAIAQAD |
| 40760 | WGLANNCHE |
| 40761 | GIWAYGCTS |
| 40762 | AACAIWKGE |
| 40763 | CGDAYDEHC |
| 40764 | DDSAQYWQV |
| 40765 | DHTACKKLC |
| 40766 | DSKADHCVN |
| 40767 | DSMADDQHL |
| 40768 | EASAFPHDQ |
| 40769 | ECHAIDQSD |
| 40770 | ECKARNSCG |
| 40771 | ECTAHGVHC |
| 40772 | EFDARVAHE |
| 40773 | EFFALDSQS |
| 40774 | EHGAVHNAN |
| 40775 | EVAASSAIH |
| 40776 | EVIAGHVGA |
| 40777 | FPSAPTEPG |
| 40778 | GKEAAYAVW |
| 40779 | GMWAQWCS |
| | N |
| 40780 | GVEAYKFPH |
| 40781 | HEQAVQKHI |
| 40782 | HNCAGWDDG |
| 40783 | HQVATQLIK |
| 40784 | HTKAYNHCV |
| 40785 | HVHAVPNFA |
| 40786 | IADAQTMQP |
| 40787 | IFVAPMPGM |
| 40788 | IHVALDQSL |
| 40789 | IRVAKKGAR |
| 40790 | ITGAMMYMG |
| 40791 | ITLACVYHD |
| 40792 | IVMADVYGN |
| 40793 | IVNACMVEK |
| 40794 | LEIALHACW |
| 40795 | LFTAFRQFT |
| 40796 | LTSAVGAGM |
| 40797 | MISAFTGSH |
| 40798 | MISAPPDTW |
| 40799 | MTFAHDTTN |
| 40800 | NEFAIQNAE |
| 40801 | NKEARGALD |
| 40802 | NTTASTDVP |
| 40803 | PCKAKEKAV |
| 40804 | QQDAMEQYF |
| 40805 | RAAAQGPPQ |
| 40806 | RFFAAELQW |
| 40807 | RLEAMYNWG |
| 40808 | SAIATHQYI |
| 40809 | SEYAAMRDS |
| 40810 | SGCAMLNMG |
| 40811 | SKCAHSFEH |
| 40812 | SNWARSSAE |
| 40813 | SRFASWEGM |
| 40814 | SRMAIDVIG |
| 40815 | STSADPHCG |
| 40816 | TQMAHMIAH |
| 40817 | TTCAHSKMG |
| 40818 | TTEADDPTT |
| 40819 | VGEAPGHWQ |
| 40820 | VVGADRCQA |
| 40821 | WEEACKLDT |
| 40822 | WHSAVQHLL |
| 40823 | WVDAFCCQC |
| 40824 | YEQAMEYET |
| 40825 | YNNADCHYH |
| 40826 | YTQAMCITA |
| 40827 | TQHALSWWS |
| 40828 | NVLARCDDI |
| 40829 | VRAAQRSEP |
| 40830 | TQNACKVRT |
| 40831 | AAKALMSCH |
| 40832 | DFHACYKCS |
| 40833 | ICAAPNSAK |
| 40834 | LSMAMQVGC |
| 40835 | MNKAMTTWA |
| 40836 | QCNALNSFV |
| 40837 | RTCAYESGT |
| 40838 | TKNAMEIQE |
| 40839 | TPNAFVFAS |
| 40840 | TQEACHHDP |
| 40841 | CMEAQGECY |
| 40842 | QMQAWYCQE |
| 40843 | GTNAYMAHK |
| 40844 | AVKAPSQMS |
| 40845 | QSDAVHEPA |
| 40846 | QGVACLDQL |
| 40847 | NVYATPVVC |
| 40848 | TNAAYEMWF |
| 40849 | DQLALTFED |
| 40850 | RTEAHLPAP |
| 40851 | LDGYMNHDC |
| 40852 | LILPMGVNN |
| 40853 | LMSVMFAAH |
| 40854 | LQDHMCHNG |
| 40855 | LQEDMSPYA |
| 40856 | LVIKMHQLW |
| 40857 | LYHEMTHAF |
| 40858 | MCAVMDIFG |
| 40859 | MMQRMVYQ |
| | C |
| 40860 | MNSLMQVAW |
| 40861 | MVQRMVYQC |
| 40862 | MVTTMGSTE |
| 40863 | NCCNMQLTF |
| 40864 | NCHVMSINL |
| 40865 | NGCGMVWQR |
| 40866 | NLAGMLPKN |
| 40867 | NMTGMPNGT |
| 40868 | QALKMENDQ |
| 40869 | QFYMMPFSL |
| 40870 | QGFFMMALM |
| 40871 | QKNHMYHQM |
| 40872 | QSDMMCHCM |
| 40873 | QTCSMLKDR |
| 40874 | RATEMAWSW |
| 40875 | SAGFMQEAA |
| 40876 | SHTRMGAFS |
| 40877 | SIAWMHLTQ |
| 40878 | SNMGMKHYE |
| 40879 | STDFMIHHF |
| 40880 | THDSMFFAC |
| 40881 | VAGNMDDHA |
| 40882 | VPYWMNTQQ |
| 40883 | VSVDMNCAS |
| 40884 | VTSLMQMGC |
| 40885 | YDTRMQTLG |
| 40886 | YRGWMNMED |
| 40887 | MSLWMATM |
| | Q |
| 40888 | AFHHMMNTS |
| 40889 | AHTIMMRDF |
| 40890 | AMDLMGDCE |
| 40891 | AMMQMMKA |
| | N |
| 40892 | ATAPMCFYQ |
| 40893 | CCCPMILFN |
| 40894 | CGEDMKICQ |
| 40895 | CMFGMVNYT |
| 40896 | CVATMHCHQ |
| 40897 | DELKMGHNI |
| 40898 | DGTPMHEYE |
| 40899 | DKMVMVFKR |
| 40900 | DKWTMQHW |
| | E |
| 40901 | DMVKMADCY |
| 40902 | DYEDMDQYY |
| 40903 | EGNIMMTQH |
| 40904 | ETSPMMTKP |
| 40905 | FQSQMPKMA |
| 40906 | FSQTMIFGA |
| 40907 | GMIGMARLA |
| 40908 | GVDPMVTHQ |
| 40909 | HKDTMHSFS |
| 40910 | HKFWMPNQH |
| 40911 | HMCWMATDK |
| 40912 | HMYGMQKHE |
| 40913 | INPKMHYAS |
| 40914 | LATTMQAFN |
| 40915 | LCEVMGCDA |
| 40916 | LENTMWKCA |
| 40917 | LGAVMTCHY |
| 40918 | LHQTMACQL |
| 40919 | LNGTMLAMS |
| 40920 | NANDMSSDR |
| 40921 | NCEPMNEWP |
| 40922 | NEPPMGLKY |
| 40923 | NESPMNDTC |
| 40924 | NITEMGAYP |
| 40925 | NTGEMAEYV |
| 40926 | PNILMMFEP |
| 40927 | PVGWMVQW |
| | P |
| 40928 | QCNHMDHVA |
| 40929 | QDNMMVRDY |
| 40930 | QISTMLARC |
| 40931 | QMATMFTCR |
| 40932 | QPISMQGQM |
| 40933 | QQVRMDRLD |
| 40934 | RDEDMHIAE |
| 40935 | RHQHMKYLD |
| 40936 | RTVDMNLKG |
| 40937 | SGLGMPNWT |
| 40938 | SGSCMWGYA |
| 40939 | SMIPMDSMP |
| 40940 | TPNNMWWM |
| | E |
| 40941 | TQVLMHATW |
| 40942 | VAQTMHYHH |
| 40943 | VKDEMHYMR |
| 40944 | VLDQMHAPP |
| 40945 | VVNSMWLMH |
| 40946 | WFHNMLATN |
| 40947 | WGMCMQRPI |
| 40948 | YRTEMNEAH |
| 40949 | YVSQMWMQC |
| 40950 | YHAPMLMCN |
| 40951 | DPVEMCSYA |
| 40952 | DRTTMVGWY |
| 40953 | LPWHMMCM |
| | P |
| 40954 | AFCRMKFGG |
| 40955 | GTEMMGDHA |
| 40956 | PCWPMMIPG |
| 40957 | GTIRMPEHR |
| 40958 | FCSPMFKQE |
| 40959 | AKAGMSYSG |
| 40960 | ACAIMKMWY |
| 40961 | AHFQMNMSP |
| 40962 | CESKMCQEP |
| 40963 | CLYNMWQCE |
| 40964 | DTLPMAMDP |
| 40965 | DYGSMAASW |
| 40966 | EDFSMPFNH |
| 40967 | ESSSMNEEK |
| 40968 | IEVGMTFPE |
| 40969 | IHQYMTAHG |
| 40970 | LDKQMWQFT |
| 40971 | MEAMMKHM |
| | E |
| 40972 | PQGIMWWTL |
| 40973 | TMWMMHSSP |
| 40974 | VGLSMHSMD |
| 40975 | VKGRMCNNF |
| 40976 | GSTHMNQEG |
| 40977 | YDYWMQAPT |
| 40978 | HNIHMWCNI |
| 40979 | MCLNMAKQG |
| 40980 | QTNTMQMLS |
| 40981 | NNCNMNVMK |
| 40982 | VLTTMWKNN |
| 40983 | FHDRMAWAR |
| 40984 | AQWYMWCW |
| | D |
| 40985 | YTELMKKAC |
| 40986 | CISPMRVHQ |
| 40987 | YASTMHSIP |
| 40988 | CEFPMGPSD |
| 40989 | LLPIMHTHW |
| 40990 | FQHRMEEDA |
| 40991 | STALMDANM |
| 40992 | EFGEMLGVD |
| 40993 | VTSHMATAC |
| 40994 | PAYRMECCE |
| 40995 | GAQSMQFSC |
| 40996 | YVIPMVPSS |
| 40997 | AAFVMNGDG |
| 40998 | FTEPMCMQG |
| 40999 | LHDSRNKSP |
| 41000 | LRDFENLIL |
| 41001 | LTEDGNWWS |
| 41002 | LVITFNQGC |
| 41003 | MGLSFNHTR |
| 41004 | MSTERNRAC |
| 41005 | PQQHTNFSK |
| 41006 | QCTVQNQFG |
| 41007 | QMELSNQWN |
| 41008 | QTVDHNITL |
| 41009 | SAKYDNVQI |
| 41010 | SAYSTNGDA |
| 41011 | SCQDVNNYH |
| 41012 | SHGYGNRFA |
| 41013 | SHYQDNIGF |
| 41014 | SSKPSNEMW |
| 41015 | TKKEINDCC |
| 41016 | TMVMNNWV |
| | A |
| 41017 | TQHSNNQAT |
| 41018 | WHKLHNRHV |
| 41019 | AHAFNNVVD |
| 41020 | AKHWFNESG |
| 41021 | AVVQANQVL |
| 41022 | AYPHRNVGL |
| 41023 | CKQQFNGQN |
| 41024 | CMNEWNKLG |
| 41025 | CMVDANHLW |
| 41026 | CNFNNNDHA |
| 41027 | CTLFSNFQY |
| 41028 | DSCKANTQS |
| 41029 | EAWMHNFN |
| | W |
| 41030 | EIYITNCTS |
| 41031 | ERHGFNHCN |
| 41032 | ESDPRNLGP |
| 41033 | ETEERNGFG |
| 41034 | GFGHSNGFL |
| 41035 | GISTFNLWK |
| 41036 | HMTEKNMGE |
| 41037 | HYMMNNYLV |
| 41038 | ICAIVNTCN |
| 41039 | ICSGVNADN |
| 41040 | IQVRWNAEL |
| 41041 | IRYILNPEF |
| 41042 | ISDCFNCAD |
| 41043 | LCAHYNPHN |
| 41044 | LHGNYNYSV |
| 41045 | LNSHINGHM |
| 41046 | LSCCTNVFC |
| 41047 | LSCMRNMSL |
| 41048 | LSDYVNDEG |
| 41049 | LVMPYNSAQ |
| 41050 | LYEERNAQM |
| 41051 | MCNGYNVSF |
| 41052 | MHVFSNVCG |
| 41053 | MQYTINYQD |
| 41054 | NETSLNQHM |
| 41055 | NFVYWNYLP |
| 41056 | NGVNINQCF |
| 41057 | NHVNANLRA |
| 41058 | NKQYFNQKD |
| 41059 | NSHDYNCFH |
| 41060 | NSLHNNEDP |
| 41061 | NTGMDNGQG |
| 41062 | NVLPFNCNF |
| 41063 | QHEYLNQKS |
| 41064 | QSQRLNGQI |
| 41065 | RTEYANHMH |
| 41066 | SFYSTNFQL |
| 41067 | SSGWLNSEN |
| 41068 | STCIDNGRT |
| 41069 | SVWWGNSAA |
| 41070 | TPMWVNMVC |
| 41071 | TVGVINLVR |
| 41072 | TYELLNPGT |
| 41073 | TYHCQNHDH |
| 41074 | VHQWTNMQF |
| 41075 | VPQTYNVEA |
| 41076 | VQDLFNQFC |
| 41077 | VVHCNNTSW |
| 41078 | WDCLLNKWR |
| 41079 | YGMYENFHC |
| 41080 | YRCHNNEKT |
| 41081 | IEEGKNALY |
| 41082 | GSATPNETP |
| 41083 | YVSLCNDGN |
| 41084 | ADHLHNVWI |
| 41085 | CHCHYNPFC |
| 41086 | CVMPSNCQD |
| 41087 | CYDMWNMTT |
| 41088 | DIQYKNHMN |
| 41089 | DKLLSNSWR |
| 41090 | DTESHNETP |
| 41091 | FKMDTNFAP |
| 41092 | ITYEGNISP |
| 41093 | LHLWRNSGE |
| 41094 | MTTHSNCGP |
| 41095 | PTKYHNDMG |
| 41096 | QLSKVNVYE |
| 41097 | RHSDHNYIG |
| 41098 | SNQHNNQSM |
| 41099 | SPGTENRGG |
| 41100 | SQMQVNPLH |
| 41101 | TLTEVNRLA |
| 41102 | TQEHINIWN |
| 41103 | TSHFKNCIM |
| 41104 | TTWEQNVMD |
| 41105 | TVCFENLGG |
| 41106 | YALLHNAMW |
| 41107 | MSARINTPD |
| 41108 | ERNMFNICH |
| 41109 | GRQSINEIA |
| 41110 | STGCYNWPQ |
| 41111 | WGQSYNYWP |
| 41112 | AVKSCNTCK |
| 41113 | QESMCNYWQ |
| 41114 | DKCLNNKFY |
| 41115 | HMAYCNKVC |
| 41116 | GAMLNNNAS |
| 41117 | GKTQYNHHN |
| 41118 | TYDTRNHCL |
| 41119 | NGNHYNAMR |
| 41120 | DYSQVNCEK |
| 41121 | GTMKHNGRA |
| 41122 | EKIRQNAAP |
| 41123 | NLERCNTMM |
| 41124 | SRYVNNGTV |
| 41125 | LRHEGYHSE |
| 41126 | MEYWPIHCQ |
| 41127 | MFLPTDHQF |
| 41128 | MQHEETHDY |
| 41129 | MREQKSHGY |
| 41130 | MTHGHSHSF |
| 41131 | NLDMVTHAE |
| 41132 | NTQGTVHIQ |
| 41133 | NVDNHDHFE |
| 41134 | PQPSEYHWN |
| 41135 | QEHQYEHQS |
| 41136 | QGPECTHNP |
| 41137 | QSKWQDHDY |
| 41138 | QSVWLEHTL |
| 41139 | QSVYDMHTP |
| 41140 | QVDGQCHTT |
| 41141 | RDSRPGHQY |
| 41142 | RQFKQTHFQ |
| 41143 | RSPHVHHQL |
| 41144 | SAHTDRHNS |
| 41145 | TSPCDMHFS |
| 41146 | TYNPWQHFE |
| 41147 | VHCQTDHDG |
| 41148 | WAHYKAHYP |
| 41149 | WQDFYIHYA |
| 41150 | WRYMWPHRQ |
| 41151 | YAVMYQHHA |
| 41152 | YCLQYAHKG |
| 41153 | AFWNPTHFD |
| 41154 | AMIPNLHME |
| 41155 | AMLGYQHQP |
| 41156 | AQDIYEHMQ |
| 41157 | ASYHWTHAG |
| 41158 | AVVIEKHDG |
| 41159 | CMKSYDHGS |
| 41160 | CNVWPCHQE |
| 41161 | DADLAAHWP |
| 41162 | DAQLGMHFI |
| 41163 | DKANCYHWL |
| 41164 | DKIPIDHFA |
| 41165 | DKIPIDHFV |
| 41166 | DVHHLTHSP |
| 41167 | EANKAFHLA |
| 41168 | EHGDEMHPP |
| 41169 | EHMNCMHCE |
| 41170 | GAGMRMHDT |
| 41171 | HKASWCHDQ |
| 41172 | HMMLRDHNN |
| 41173 | ICSIYGHHQ |
| 41174 | IDSQIYHML |
| 41175 | LCAEFPHGR |
| 41176 | LITQPVHQQ |
| 41177 | LQAGGEHNW |
| 41178 | LTLESPHPA |
| 41179 | LYKWIVHNS |
| 41180 | LYYITEHST |
| 41181 | MAVSQSHQF |
| 41182 | MAVWVKHQH |
| 41183 | MSSMLEHSW |
| 41184 | MYGFYGHDM |
| 41185 | NAYQNSHYP |
| 41186 | QLYGRRHWH |
| 41187 | QMMDFQHPA |
| 41188 | QNYWGRHPA |
| 41189 | QSHYRMHSA |
| 41190 | QVNHVRHWS |
| 41191 | RHICFQHCK |
| 41192 | RIEIGQHYV |
| 41193 | RSEVSSHLD |
| 41194 | SANWFTHDK |
| 41195 | SVSLKVHHE |
| 41196 | SYVTTQHST |
| 41197 | TASGIDHPK |
| 41198 | TQAEYSHKS |
| 41199 | TTWGTGHMA |
| 41200 | VFNLYSHSH |
| 41201 | VIKPFAHHG |
| 41202 | YASYKCHGD |
| 41203 | YVQSAQHQQ |
| 41204 | YYASLDHYL |
| 41205 | NFWNQVHCV |
| 41206 | MATNKYHIQ |
| 41207 | QYYNYCHMN |
| 41208 | GCMCIRHAY |
| 41209 | YIYFALHTH |
| 41210 | WFDRSWHIL |
| 41211 | NIAMPAHIP |
| 41212 | HDPLAGHWP |
| 41213 | QRQGQLHYW |
| 41214 | IACISMHNH |
| 41215 | AQEHLDHCL |
| 41216 | ECTMRMHSH |
| 41217 | EDSFSFHTI |
| 41218 | EYCETMHTN |
| 41219 | FMPYCVHSG |
| 41220 | HSKNTVHKR |
| 41221 | IMGIHLHPA |
| 41222 | IRNIAKHAL |
| 41223 | LDKRQDHNF |
| 41224 | MQAEACHRW |
| 41225 | NTHLFHHYL |
| 41226 | QHTSAFHTM |
| 41227 | WHKCLSHQD |
| 41228 | WRKCLSHQD |
| 41229 | NAYYDSHCE |
| 41230 | HDPPPMHWL |
| 41231 | RCQLFDHDE |
| 41232 | LGKVQTHIR |
| 41233 | DVIHTTHSG |
| 41234 | TKGRNDHME |
| 41235 | NSTRWYHTG |
| 41236 | DTDNKPHFA |
| 41237 | QGYSHLHAL |
| 41238 | CQAGTDHFP |
| 41239 | CASSKSHYL |
| 41240 | ALGNRHHQC |
| 41241 | VQYYYGHMQ |
| 41242 | GVFYVGHWQ |
| 41243 | LVQQDWRFW |
| 41244 | MCAYLHMFH |
| 41245 | MEKVKCSFA |
| 41246 | PKNWAPMFT |
| 41247 | QQDIYIDFF |
| 41248 | QTEHLWMFH |
| 41249 | SGHWNDYFH |
| 41250 | STIPIWNFL |
| 41251 | TEQFSRLFA |
| 41252 | TQDSKMGFG |
| 41253 | TVAWATAFE |
| 41254 | VKKLAKYFH |
| 41255 | VKVDKSQFD |
| 41256 | VVNIRGSFQ |
| 41257 | AFEVSESFE |
| 41258 | AGEMDEAFG |
| 41259 | AMELADDFA |
| 41260 | AVDHCFKFE |
| 41261 | DFKKPSLFR |
| 41262 | DMQTICRFP |
| 41263 | GMGWQVSFN |
| 41264 | GPIPSEVFP |
| 41265 | HCYIKEGFQ |
| 41266 | HNMPSGQFT |
| 41267 | HYESRLDFY |
| 41268 | ICKSDPVFD |
| 41269 | LEPCTDEFG |
| 41270 | LIMRISPFD |
| 41271 | LSGSIMEFN |
| 41272 | MVSENSTFW |
| 41273 | MYSRTIPFH |
| 41274 | NKHEVGCFG |
| 41275 | NKYEVGCFG |
| 41276 | NMHWRYQFE |
| 41277 | PYPTHSMFS |
| 41278 | QDSHVHSFF |
| 41279 | QGQYWHYFK |
| 41280 | SCGHQTRFG |
| 41281 | SCGHQTWFG |
| 41282 | SDHYCGTFY |
| 41283 | SHMMSHAFW |
| 41284 | SNSLYRAFL |
| 41285 | SSVCTQIFT |
| 41286 | TGEMDEAFG |
| 41287 | TNDIWWQFS |
| 41288 | TRTELANFF |
| 41289 | VCWLWFYFG |
| 41290 | VGICHPEFW |
| 41291 | WVTNQSDFA |
| 41292 | YPQCAQWFW |
| 41293 | HQKMTYAFT |
| 41294 | VRWGHTCFY |
| 41295 | NSAECREFQ |
| 41296 | WKNNQKWFK |
| 41297 | DIGSYKGFQ |
| 41298 | DSAHYCTFT |
| 41299 | EKSTILPFA |
| 41300 | EKSTILPFT |
| 41301 | EVEYGCWFE |
| 41302 | GNMRQSRFP |
| 41303 | HGLHEDSFY |
| 41304 | TNNYPMPFD |
| 41305 | DMPPLHSFC |
| 41306 | ASTMKSIFE |
| 41307 | SLASEVQFQ |
| 41308 | DLTTDTIFQ |
| 41309 | SADRTAMFH |
| 41310 | PTYPYEWFQ |
| 41311 | NMKPKDVFR |
| 41312 | SATIRFYFN |
| 41313 | FVPWCHSFG |
| 41314 | ITEQYDAFA |
| 41315 | NTMCQLNFC |
| 41316 | LYQKDQRFS |
| 41317 | LFGTHPGQA |
| 41318 | LQELSSQYA |
| 41319 | LTVNLMKIA |
| 41320 | NAIDCMSGA |
| 41321 | NAWIDWSLA |
| 41322 | NQGTSAWGA |
| 41323 | NSLPFGMVA |
| 41324 | PFNKSKGLA |
| 41325 | PNQVGTQGA |
| 41326 | QDQQVHEGA |
| 41327 | QLMIQGSYA |
| 41328 | QSAYLIMVA |
| 41329 | QTTFAERHA |
| 41330 | SDHGRIMTA |
| 41331 | SLAHWDGRA |
| 41332 | SSGWTQQMA |
| 41333 | TFIMLGADA |
| 41334 | VDYYSPYLA |
| 41335 | VGCIAQAAA |
| 41336 | VNDCNWCEA |
| 41337 | VRTPWQTVA |
| 41338 | WFNVKKTAA |
| 41339 | YAPQQHMWA |
| 41340 | YSQKQCPTA |
| 41341 | ACYQQCVSA |
| 41342 | AKSSIEYSA |
| 41343 | ALWDKFEAA |
| 41344 | CDYHTLSPA |
| 41345 | CFENLQVMA |
| 41346 | CRDWDGFNA |
| 41347 | CTTDTPQNA |
| 41348 | CYHECQGCA |
| 41349 | DIFFKCEWA |
| 41350 | DVGGRAVQA |
| 41351 | EATDLCLNA |
| 41352 | EATDLCPNA |
| 41353 | EDNHYQMIA |
| 41354 | EFYNYEIMA |
| 41355 | EGGSNMMNA |
| 41356 | EKAMLLNGA |
| 41357 | EKTCVTREA |
| 41358 | EQAMWMYPA |
| 41359 | EQGHCEVAA |
| 41360 | ESKWYYVMA |
| 41361 | EVERDKVGA |
| 41362 | FDEKRSESA |
| 41363 | FPANYHSNA |
| 41364 | FVSTQMPIA |
| 41365 | GPNDWMFDA |
| 41366 | GYWLWMYVA |
| 41367 | HCDDVLWCA |
| 41368 | HSAYFGVGA |
| 41369 | IEELGKPRA |
| 41370 | INGMKSAMA |
| 41371 | INVPDLLGA |
| 41372 | ITSIRMYQA |
| 41373 | IVQPYHVEA |
| 41374 | LHQPRMDCA |
| 41375 | LIYHTPVMA |
| 41376 | LSSCRGLIA |
| 41377 | LTIRDYGEA |
| 41378 | LVNVRYMHA |
| 41379 | MHEPILKYA |
| 41380 | MICWWKQPA |
| 41381 | MMNHSQLYA |
| 41382 | MQHPYMQM |
| | A |
| 41383 | MSTVSGSYA |
| 41384 | NGFMAAVMA |
| 41385 | NHADYMRNA |
| 41386 | NMWPTFRAA |
| 41387 | NTDEESGMA |
| 41388 | NTMMLQSAA |
| 41389 | QAPLKRAAA |
| 41390 | QCNRCDQYA |
| 41391 | QFIQYGTCA |
| 41392 | QIGLKMTPA |
| 41393 | QLNPWFYYA |
| 41394 | REISVEAVA |
| 41395 | SAKITWASA |
| 41396 | SIVEFESAA |
| 41397 | STHMATQLA |
| 41398 | TKSSIEYSA |
| 41399 | TNLRQHTNA |
| 41400 | TVSDGDWIA |
| 41401 | TVVLTLKEA |
| 41402 | VTSWFHEIA |
| 41403 | VTTVSGGCA |
| 41404 | VVMCKSRDA |
| 41405 | WVMHYYQYA |
| 41406 | YSSPDLRPA |
| 41407 | TVLVPVQAA |
| 41408 | EVGIKYRAA |
| 41409 | EVNWQCQYA |
| 41410 | GSMFNPGAA |
| 41411 | MTNWFPACA |
| 41412 | MVKTSCKPA |
| 41413 | MVKTVYPDA |
| 41414 | PPEEQCGMA |
| 41415 | QFTETWPCA |
| 41416 | RIIWPDVVA |
| 41417 | SSDTPANQA |
| 41418 | TFVECIQYA |
| 41419 | TGHFFTTAA |
| 41420 | VGYGCKGYA |
| 41421 | VSWSRMEQA |
| 41422 | YRLHCCNRA |
| 41423 | MRVQVFSDA |
| 41424 | RTESWSSLA |
| 41425 | QKWMQMDG |
| | A |
| 41426 | TAAGYIVDA |
| 41427 | TQQTADRWA |
| 41428 | FQIRQDNLA |
| 41429 | NTGMEGQMA |
| 41430 | IMTMQMDTA |
| 41431 | EYSSQHPYA |
| 41432 | QPFCFSIGA |
| 41433 | MGVPPLMQA |
| 41434 | VFGSTRSAA |
| 41435 | ATNYTMEPA |
| 41436 | HIHMSDRGA |
| 41437 | GSHDYYQSA |

### Example 16

AAV5 Variants with Tissue Tropism in Lymph Node

This example describes engineered AAV5 variants with tissue tropism in lymph node that were discovered using the methods and systems described in EXAMPLE 1-EXAMPLE 2.

CNS, liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive lymph node tropism. The results are shown in FIG. 18G which shows the positional frequency of each amino acid in the 581 to 589 region of AAV5 VP1 in lymph node tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in lymph node over the frequency of that given amino acid residue occurring at the specified position in variants identified in all other harvested tissues was analyzed to identify a set of sequence rules for capsids that preferentially target lymph node tissue. With reference to TABLE 31 below, and SEQ ID NO: 2 (AAV5 VP1), the following amino acids can, thus, be independently mutated, in any combination, at any one or more positions Xaa1-Xaa9, to provide a VP1 capsid with enhanced lymph node tropism as compared wildtype AAV5 VP1 capsid (SEQ ID NO: 1), where lymph node tropism here refers to properties that are preferred for lymph node transduction over properties that are preferred for transduction of all other harvested tissues.

TABLE 31
Lymph Node Tropism Rules

Xaa1 is selected from A, D, E, Q, S, or T
Xaa1 is selected from D, E, or T
Xaa1 is E
Xaa2 is selected from A, H, I, S, T, or V
Xaa2 is selected from I, T, or V
Xaa2 is V
Xaa3 is selected from A, E, H, I, T, or V
Xaa3 is selected from A, I, T, or V
Xaa3 is T
Xaa4 is selected from A, D, E, or P
Xaa4 is selected from D, or E
Xaa4 is E
Xaa5 is selected from I, L, M, V, or Y
Xaa5 is selected from I, L, V, or Y
Xaa5 is L
Xaa6 is selected from D, E, I, N, or Q
Xaa6 is selected from D, E, or I
Xaa6 is D
Xaa7 is selected from A, E, G, Q, or V
Xaa7 is A, Q, or V
Xaa7 is V
Xaa8 is selected from F, G, M, or W
Xaa8 is selected from For W
Xaa8 is W
Xaa9 is selected from I, P, T, or Y
Xaa9 is I or P

TABLE 32 below provides sequences of the 581 to 589 region for variant AAV VP1 capsid polypeptides that were found in lymph node tissue and comport to one or more of the rules provided in TABLE 31. The present disclosure, thus, provides for rAAVs composed of engineered AAV5 VP1 capsid polypeptides having a SEQ ID NO: 2, wherein the Xaa1 to Xaa9 region is any one of SEQ ID NO: 19118-SEQ ID NO: 20, as disclosed in TABLE 32. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 32
Sequences of the 581 to 589 Region in AAV5
VP1 Capsid Polypeptide that Drive Lymph
Node Tissue Tropism

| SEQ | |
|---|---|
| ID | 581-589 |
| NO | Sequence |
| 19118 | ETTMWNSWA |
| 19119 | ENTMWQSSQ |
| 19120 | ERQAHNHYA |
| 19121 | EAKSRPQYA |
| 19122 | ECQQDDTDI |
| 19123 | EDICHWHHT |
| 19124 | EECIMQRSK |
| 19125 | EEERTCKHC |
| 19126 | EEFARIPGH |
| 19127 | EEPRCVNPC |
| 19128 | EFFCQADKA |
| 19129 | EGAGLYEFA |
| 19130 | EGPELTCDK |
| 19131 | EGTLRYTMS |
| 19132 | EHDTQDISV |
| 19133 | EHHPLGVMG |
| 19134 | EHHYCVPWS |
| 19135 | EHILTAERI |
| 19136 | EIAAKIFCR |
| 19137 | EIILIWKQC |
| 19138 | EIMSYPHTN |
| 19139 | EIQHVSHEH |
| 19140 | EKHGCDKAE |
| 19141 | ELNHKWHSV |
| 19142 | EMALVVSNQ |
| 19143 | EMHQFMECQ |
| 19144 | EMRFTNFCY |
| 19145 | ENIHNNDCT |
| 19146 | ENKEKEGQW |
| 19147 | ENTMAIIEH |
| 19148 | EPILYHEHK |
| 19149 | EPIQIFNDA |
| 19150 | EQHRDTMRP |
| 19151 | EQNACDHYG |
| 19152 | EQVFGFWNY |
| 19153 | ERMETFDCP |
| 19154 | ESCEDEGIL |
| 19155 | ESKASGANG |
| 19156 | ESMLPIMGR |
| 19157 | ESVPLEYAN |
| 19158 | ETCCFTNMG |
| 19159 | ETCGQGGYT |
| 19160 | ETDHCESWY |
| 19161 | ETPIEWDEQ |
| 19162 | EVEVHSTAL |
| 19163 | EVHIAPSAC |
| 19164 | EVKRITEQS |
| 19165 | EVKRWMMVD |
| 19166 | EVMAMVDVA |
| 19167 | EVPQNELNV |
| 19168 | EVRSIEYWN |
| 19169 | EVWHTKILI |
| 19170 | EWAFNYNQN |
| 19171 | EYKMFEKYE |
| 19172 | EDHWHGPGD |
| 19173 | FVALKAVID |
| 19174 | FVAPVDSFY |
| 19175 | FVFEGPMHQ |
| 19176 | FVKVRIWTQ |
| 19177 | FVQPTFIRH |
| 19178 | FVWYGPRNH |
| 19179 | GVACVQETD |
| 19180 | GVANFMLNN |
| 19181 | GVCEENEWM |
| 19182 | GVCQMCCWP |
| 19183 | GVDARHAQR |
| 19184 | GVEFCLTNG |
| 19185 | GVEWPPFMQ |
| 19186 | GVFMTWQWL |
| 19187 | GVHGINVMM |
| 19188 | GVHIMQCQF |
| 19189 | GVHIYACHY |
| 19190 | GVHQVPTWN |
| 19191 | GVLQMDWQE |
| 19192 | GVMQHWTCD |
| 19193 | GVMWDGVAP |
| 19194 | GVQMTVRQA |
| 19195 | GVTYAQRIC |
| 19196 | GVVFCDQKD |
| 19197 | GVVLFQGGA |
| 19198 | GVVNSMQTK |
| 19199 | GVVQQGAMI |
| 19200 | HVFVMDGPI |
| 19201 | HVIWDSMKL |
| 19202 | HVMDHVATS |
| 19203 | IVDFENRHP |
| 19204 | IVDPIFRQT |
| 19205 | IVFEGGIAS |
| 19206 | IVGAPCLVT |
| 19207 | IVGDCQFHV |
| 19208 | IVGSNTYGY |
| 19209 | IVHNVEYSN |
| 19210 | IVHSQPNSV |
| 19211 | IVLQLQQAK |
| 19212 | IVNALEFCK |
| 19213 | IVNNAQMHE |
| 19214 | IVQMHYINQ |
| 19215 | IVQVESCQN |
| 19216 | IVSARDTHN |
| 19217 | KVDGMDIEG |
| 19218 | KVDPPGIFW |
| 19219 | KVDQGLCQP |
| 19220 | KVDWDGNID |
| 19221 | KVEPHHPAD |
| 19222 | KVNPKEFQD |
| 19223 | KVPICAHKQ |
| 19224 | KVQICDFWC |
| 19225 | LVCLCHSCA |
| 19226 | LVDQYMWDN |
| 19227 | LVDRVKHVD |
| 19228 | LVDYWAWNG |
| 19229 | LVEQERMGV |
| 19230 | LVFAKLDQN |
| 19231 | LVGICDLCP |
| 19232 | LVGSTSHVT |
| 19233 | LVMHQQRNY |
| 19234 | LVYQHDTAP |
| 19235 | LVYTELSGA |
| 19236 | MVATESHDK |
| 19237 | MVDDPGQQF |
| 19238 | MVDHWGDWN |
| 19239 | MVDHYICMY |
| 19240 | MVDSFGKYL |
| 19241 | MVECEGEQY |
| 19242 | MVEDSTCHW |
| 19243 | MVEEANVPC |
| 19244 | MVEEGSWNI |
| 19245 | MVEGRQDLT |
| 19246 | MVENCDLPP |
| 19247 | MVEWGGSNQ |
| 19248 | MVGCFYTWT |
| 19249 | MVGYQYPAA |
| 19250 | MVHEAELYA |
| 19251 | MVKEDITEH |
| 19252 | MVKEQWQCT |
| 19253 | MVLPANGFQ |
| 19254 | MVNALTDYG |
| 19255 | MVNPWVVFY |
| 19256 | MVNQAWQTD |
| 19257 | MVQPMPQHH |
| 19258 | MVSFNYEIG |
| 19259 | MVTKEYMCC |
| 19260 | MVTMGYWSS |
| 19261 | MVTPYWNQY |
| 19262 | MVYPKRGEN |
| 19263 | MVYSGQQEF |
| 19264 | NVAFAHCGG |
| 19265 | NVDDHPTMC |
| 19266 | NVDMRNVES |
| 19267 | NVDTPSGNQ |
| 19268 | NVDWDSECS |
| 19269 | NVECCLNRP |
| 19270 | NVEEKWEWW |
| 19271 | NVEFRYFYN |
| 19272 | NVESFPHEQ |
| 19273 | NVEWWGHCA |
| 19274 | NVFATNCWV |
| 19275 | NVGAHFAPF |
| 19276 | NVGGPCYNL |
| 19277 | NVGGQNMEH |
| 19278 | NVGIGHGGN |
| 19279 | NVGIHFAQQ |
| 19280 | NVGNEHIAY |
| 19281 | NVHIALEQH |
| 19282 | NVIQHAIGG |
| 19283 | NVKFSIEHT |
| 19284 | NVLGTLVHV |
| 19285 | NVMDCESCC |
| 19286 | NVSYEQNFM |
| 19287 | PVATALRMP |
| 19288 | PVNLNCTVS |
| 19289 | PVSLCRFGG |
| 19290 | PVTGENHMN |
| 19291 | QVALRGATC |
| 19292 | QVDDCGHLF |
| 19293 | QVDMELPCW |
| 19294 | QVETTMIGH |
| 19295 | QVIQNVWER |
| 19296 | QVIQRRNVM |
| 19297 | QVMIECRQS |
| 19298 | QVMNGSPKT |
| 19299 | QVQIGFEQG |
| 19300 | QVVTWGYEY |
| 19301 | QVYFWGTHS |
| 19302 | RVCDKNLDL |
| 19303 | RVDEKLDHD |
| 19304 | RVDTNMRDR |
| 19305 | RVEHWVNMS |
| 19306 | RVNEWGAYG |
| 19307 | RVPLYPYWT |
| 19308 | RVSPWDQWF |
| 19309 | RVVMPLGSL |
| 19310 | SVAYKWDAF |
| 19311 | SVDACDYDD |
| 19312 | SVDDCMHSA |
| 19313 | SVDNDRAWP |
| 19314 | SVEAKSATC |
| 19315 | SVEFHEDFS |
| 19316 | SVEGANAEV |
| 19317 | SVEGVIIDR |
| 19318 | SVEYYSMPQ |
| 19319 | SVFQTHCDW |
| 19320 | SVGPLQYWK |
| 19321 | SVGWGWMMG |
| 19322 | SVHEWDPCT |
| 19323 | SVHGKVSWF |
| 19324 | SVHTIWPMA |
| 19325 | SVICWTHMA |
| 19326 | SVIYNEHVT |
| 19327 | SVMKQELSV |
| 19328 | SVNMYMLPD |
| 19329 | SVSMASAHG |
| 19330 | SVTACPNWN |
| 19331 | SVTASYGFS |
| 19332 | SVVEDAMRG |
| 19333 | SVVPTNNDM |
| 19334 | SVYAPTKWG |
| 19335 | SVYWPTTEF |
| 19336 | TVADMFFDG |
| 19337 | TVAHVADCY |
| 19338 | TVAPCNQGL |
| 19339 | TVATHCFIH |
| 19340 | TVAVWWRNA |
| 19341 | TVDAIQGWQ |
| 19342 | TVDMRHFFT |
| 19343 | TVDSEHRLC |
| 19344 | TVEAMHLGP |
| 19345 | TVEDGQWSW |
| 19346 | TVEFTYGWC |
| 19347 | TVEPTNYDG |
| 19348 | TVFHSTGVG |
| 19349 | TVGICFCPV |
| 19350 | TVGLLEMTR |
| 19351 | TVGTGCDTC |
| 19352 | TVGWYQHDW |
| 19353 | TVHNCLTMG |
| 19354 | TVLSTTQKD |
| 19355 | TVMHHDGRL |
| 19356 | TVMSWQQHE |
| 19357 | TVMWQTQIG |
| 19358 | TVNIILQGS |
| 19359 | TVQMYQDLE |
| 19360 | TVSESRKHN |
| 19361 | TVTDEDDCN |
| 19362 | TVTIAVQNM |
| 19363 | TVVQWHAMI |
| 19364 | TVWTEVSPP |
| 19365 | VVDPRPDWF |
| 19366 | VVDRWDHWM |
| 19367 | VVDSNFCSM |
| 19368 | VVGCDKSDA |
| 19369 | VVHEAWCDT |
| 19370 | VVHNFLDQS |
| 19371 | VVKEFTGHY |
| 19372 | VVKICGCCR |
| 19373 | VVMLQSGMT |
| 19374 | VVMREWRCF |
| 19375 | VVNQGVACT |
| 19376 | VVQHIWWGI |
| 19377 | VVRWPGSEW |
| 19378 | VVSRNEVGA |
| 19379 | VVTFTNILQ |
| 19380 | VVVCRPVAP |
| 19381 | VVYEKQDHM |
| 19382 | VVYPLAPYR |
| 19383 | VVYSNICVE |
| 19384 | WVASQGAQC |
| 19385 | YVANVPVLW |
| 19386 | YVLGRHCVF |
| 19387 | YVQIKGLQL |
| 19388 | YVTALATYD |
| 19389 | SVTNGSTKH |
| 19390 | LVCIRAGVE |
| 19391 | KVNCRMIIM |
| 19392 | GVWMQGFHP |
| 19393 | AVHTADVCC |
| 19394 | DVKSWAVCD |
| 19395 | SVLHVQALA |
| 19396 | AVSPHWGNY |
| 19397 | CVVKRMAEF |
| 19398 | CVVNMEYCL |
| 19399 | CVWSRHQIC |
| 19400 | DVRLIGHEA |
| 19401 | DVVRQWQAP |
| 19402 | FVEEDLYEE |
| 19403 | FVHMWSRLC |
| 19404 | FVRPLEEFY |
| 19405 | HVFQGDYIA |
| 19406 | HVGNLTAFN |
| 19407 | IVANAQIPG |
| 19408 | IVGYQNCQS |
| 19409 | IVLGQMTCT |
| 19410 | IVSEWEDWQ |
| 19411 | IVSNSVCRV |
| 19412 | KVCVVAGPM |
| 19413 | KVEVKRDYW |
| 19414 | KVVDRETYS |
| 19415 | KVYEIGEAP |
| 19416 | MVEWEEEWD |
| 19417 | MVILRGMSF |
| 19418 | MVLPTKLWD |
| 19419 | MVSVVQEPN |
| 19420 | NVLSEVYGH |
| 19421 | PVKDCILWS |
| 19422 | QVCSDWCEC |
| 19423 | QVDTRCYGG |
| 19424 | QVMMDDSNV |
| 19425 | QVSCNVEPK |
| 19426 | FPTQMKMCN |
| 19427 | FPTWDKPPT |
| 19428 | FTTCFSIMV |
| 19429 | FTTFWVPQQ |
| 19430 | FTTLHNMEN |
| 19431 | FYTCELACV |
| 19432 | GITTIWPDH |
| 19433 | GLTADLWHY |
| 19434 | GMTYVSGHH |
| 19435 | GNTLIVTGP |
| 19436 | GSTTQHDGT |
| 19437 | GWTPHTLLR |
| 19438 | GYTANFYTN |
| 19439 | HGTTIASGP |
| 19440 | HKTIDDIFT |
| 19441 | HNTEPPGMP |
| 19442 | HPTNMSACE |
| 19443 | HSTGSGTFH |
| 19444 | HSTPFVFNS |
| 19445 | IATNQSAIL |
| 19446 | IGTAYPQWR |
| 19447 | IGTIHPQAQ |
| 19448 | IGTVTVGAY |
| 19449 | IITMMCRSD |
| 19450 | IMTSVDSQR |
| 19451 | IRTRELDQY |
| 19452 | ISTEKNESY |
| 19453 | ISTLFSSGD |
| 19454 | ITTKYDCMG |
| 19455 | ITTPFYKVG |
| 19456 | IWTRADLGR |
| 19457 | KMTVRQSDS |
| 19458 | KTTDDTVGE |
| 19459 | KWTQFGWMD |
| 19460 | KWTRIGDSA |
| 19461 | LGTQEQGMP |
| 19462 | LHTRTQCEP |
| 19463 | LNTGYMWNQ |
| 19464 | LPTSVNMMH |
| 19465 | LQTAIPFWP |
| 19466 | LSTTIDKDP |
| 19467 | LTTAVAIRE |
| 19468 | LTTQLFLQE |
| 19469 | LTTQYICGP |
| 19470 | LTTWNTYVN |
| 19471 | LWTQISEFA |
| 19472 | MATEGACNP |
| 19473 | MATMFTNWC |
| 19474 | MITCIDGIF |
| 19475 | MPTLCECGL |
| 19476 | MSTMSMDSY |
| 19477 | MSTTELVAE |
| 19478 | MWTGQLRQC |
| 19479 | MYTQWSKAP |
| 19480 | NATNSQNMY |
| 19481 | NATPYKAEY |
| 19482 | NFTVTNLQM |
| 19483 | NITLGAACE |
| 19484 | NNTNQCQNC |
| 19485 | NQTEMSSNG |
| 19486 | NRTCNAGEW |
| 19487 | NRTGPAAWA |
| 19488 | NSTAFFRDQ |
| 19489 | NSTIQGGAW |
| 19490 | NTTEISRLP |
| 19491 | NYTIRVQDL |
| 19492 | NYTNCEFGN |
| 19493 | PATTADMHY |
| 19494 | PPTHQMWYC |
| 19495 | QATSAMIPS |
| 19496 | QCTCEGEAQ |
| 19497 | QDTELYCCM |
| 19498 | QITQMGRQE |
| 19499 | QLTWQAHSW |
| 19500 | QMTPGWWKT |
| 19501 | QNTCLPVFY |
| 19502 | QTTSAMING |
| 19503 | QYTHGTCFS |
| 19504 | RYTGTWQDC |
| 19505 | SATYTEMQS |
| 19506 | SITHNDHGW |
| 19507 | SKTCEIGKT |
| 19508 | SNTAYFNGS |
| 19509 | SSTAEPNGR |
| 19510 | STTAIDCMR |
| 19511 | SWTACTPCH |
| 19512 | SYTYRLTDQ |
| 19513 | THTEFDVFN |
| 19514 | TKTAFGDMG |
| 19515 | TNTAFGCYG |
| 19516 | TSTEYKMGL |
| 19517 | TTTMLHRIN |
| 19518 | TTTTQDSQR |
| 19519 | TTTYQALNA |
| 19520 | TYTCNKWFR |
| 19521 | VATKFETNT |
| 19522 | VATMAEDNP |
| 19523 | VATTDGPVY |
| 19524 | VCTNLAHGA |
| 19525 | VDTIFTDLH |
| 19526 | VGTPLDTSV |
| 19527 | VITVFMEQN |
| 19528 | VKTPGATDF |
| 19529 | VPTNTFDTH |
| 19530 | VSTAVMPEG |
| 19531 | VSTGLQDAC |
| 19532 | VSTTGPIDG |
| 19533 | VTTEMPMME |
| 19534 | VTTGYQCAS |
| 19535 | VTTTTKVSN |
| 19536 | VWTQEMWDA |
| 19537 | WDTVPKIRD |
| 19538 | WFTGPWAMH |
| 19539 | WRTQCQSEK |
| 19540 | WYTYNMCER |
| 19541 | YATFQDCNA |
| 19542 | YATNQCYNG |
| 19543 | YCTDQSETS |
| 19544 | YITKGNLAR |
| 19545 | YMTFKGYHG |
| 19546 | YPTKDPFKN |
| 19547 | YSTQCNTMK |
| 19548 | YTTDDGMSP |
| 19549 | YTTHRDTVT |
| 19550 | YTTVDNSLQ |
| 19551 | KQTVFVDQE |
| 19552 | RITQGESGM |
| 19553 | ISTKWDYIA |
| 19554 | QQTKNNSTD |
| 19555 | WCTQFDEYT |
| 19556 | KITVHDRIP |
| 19557 | DMTLQSVSS |
| 19558 | PLTIEVNCT |
| 19559 | QCTAHNTCM |
| 19560 | FNLEFSPTL |
| 19561 | FPQESNHDG |
| 19562 | GAVEIDCLY |
| 19563 | GHAEQWFES |
| 19564 | GHGEFMRDA |
| 19565 | GHVEVCTLS |
| 19566 | GIGEEMFWD |
| 19567 | GSPEVSVAS |
| 19568 | GWSEAHKSD |
| 19569 | GYYEVVKMY |
| 19570 | HGYEIQFFQ |
| 19571 | HHDEKLKML |
| 19572 | HMDELIYCG |
| 19573 | HNKEKWPTE |
| 19574 | IACESDESW |
| 19575 | IHYEHGMQC |
| 19576 | ILLEFQAPM |
| 19577 | IQQERNRYH |
| 19578 | ISAEHIFAG |
| 19579 | ISFEWLELN |
| 19580 | ISSEAIFYP |
| 19581 | ITMEILGQN |
| 19582 | KACESIQDL |
| 19583 | KAEEIENMC |
| 19584 | KAIESMLFM |
| 19585 | KAMESHDCC |
| 19586 | KFEEVHASG |
| 19587 | KHPEQYRKY |
| 19588 | KHVEALKAY |
| 19589 | KKYEECPLA |
| 19590 | KMVEDEPSA |
| 19591 | KNEEDYALY |
| 19592 | KPVEIDMEG |
| 19593 | KQYELNNWC |
| 19594 | KSSECGWAC |
| 19595 | KTIEQTIPH |
| 19596 | KTYERYQCA |
| 19597 | LAKELCCIQ |
| 19598 | LANEKDSMC |
| 19599 | LFREKMFNP |
| 19600 | LIVEHDCWF |
| 19601 | LKCEYPPMS |
| 19602 | LLDEVRQAG |
| 19603 | LLWECEKGV |
| 19604 | LTLEQSVPM |
| 19605 | LWYEVMIWY |
| 19606 | LYKENWQFP |
| 19607 | LYQENQHEW |
| 19608 | MFAEYSHIG |
| 19609 | MFEEKNLHT |
| 19610 | MHKECEACR |
| 19611 | MHMEMNYSE |
| 19612 | MIAEFVSNY |
| 19613 | MNEEQEHGA |
| 19614 | MPDEQQQMW |
| 19615 | MQAELTMSA |
| 19616 | MRHEEQRLK |
| 19617 | MRHEIVAMY |
| 19618 | MSAEVLLMT |
| 19619 | MSCELTGMH |
| 19620 | MYVEYTVGM |
| 19621 | NAVEILLCG |
| 19622 | NCNEPKYAA |
| 19623 | NEYECMCWY |
| 19624 | NGDEFTVHG |
| 19625 | NHAEFLSGC |
| 19626 | NHGEYFDWF |
| 19627 | NHYEMQDWE |
| 19628 | NKLENCIGM |
| 19629 | NNHEQEWRN |
| 19630 | NSREAEMLR |
| 19631 | NTHEFKFQF |
| 19632 | NTHEFMMQD |
| 19633 | NYDEKHVCH |
| 19634 | PFFERNIRE |
| 19635 | PKFEFYEMI |
| 19636 | PMEEMMHFC |
| 19637 | PTNELYDNE |
| 19638 | PYPEASVKM |
| 19639 | PYREGFRSP |
| 19640 | QALECTCQR |
| 19641 | QARECEAMD |
| 19642 | QASEHDTVV |
| 19643 | QHNEKPMDA |
| 19644 | QHYEFQKHC |
| 19645 | QIDEGMYSE |
| 19646 | QINEFAEHF |
| 19647 | QMREIQAEQ |
| 19648 | QNPEVHFSK |
| 19649 | QSEECMECR |
| 19650 | QSNEDLADI |
| 19651 | RAMEMQDQG |
| 19652 | RCNEYTGEK |
| 19653 | RIIEQDEIT |
| 19654 | RINEQDECM |
| 19655 | RWMELENAT |
| 19656 | SAGEWGQMW |
| 19657 | SIDEFQENW |
| 19658 | SPDEFGTFR |
| 19659 | SPHEIFVWN |
| 19660 | SSEEWCQCE |
| 19661 | SSVEGIRER |
| 19662 | SWAENKQSF |
| 19663 | TAEEIWYER |
| 19664 | TCCEPSWND |
| 19665 | TGNELYCWT |
| 19666 | THAEFEHMH |
| 19667 | TKMEYQGAT |
| 19668 | TMQEFVVKG |
| 19669 | TMYETLLMG |
| 19670 | TQNEHGKYD |
| 19671 | TREEDDKVN |
| 19672 | TRMEQPSGR |
| 19673 | TSNEMKKHW |
| 19674 | TTEEGQTFR |
| 19675 | TWGENGAWC |
| 19676 | VCEEYYQMR |
| 19677 | VFEEDAYGL |
| 19678 | VFLEGMHFP |
| 19679 | VKNEQTVFF |
| 19680 | VLGELGDMG |
| 19681 | VSPECPSFT |
| 19682 | WGAEKQWAF |
| 19683 | WGHEIMTEW |
| 19684 | WHDEKSRYL |
| 19685 | WIMEENAGQ |
| 19686 | WMIEMWNCM |
| 19687 | YADECAYGR |
| 19688 | YAGEHDMGQ |
| 19689 | YANEPQHHF |
| 19690 | YMVEYAKYG |
| 19691 | YNEEEWFFA |
| 19692 | YQVERGASA |
| 19693 | MGKEYSQCF |
| 19694 | TYEEENGFL |
| 19695 | NTCEGWSHF |
| 19696 | GLFEGSPGA |
| 19697 | MWHELIADQ |
| 19698 | NQREWHGLA |
| 19699 | GDYQLLNSP |
| 19700 | GFIDLNSRR |
| 19701 | GIDWLDQYG |
| 19702 | GIMLLPNGH |
| 19703 | GSLTLLEQT |
| 19704 | GYVSLWSMY |
| 19705 | HCVALMMDR |
| 19706 | HEEVLIAGS |
| 19707 | HGLTLKWDC |
| 19708 | HHESLGMHM |
| 19709 | HMEALMTNV |
| 19710 | HTGFLTKGM |
| 19711 | HTICLAVTP |
| 19712 | HTWQLHNWN |
| 19713 | IDLNLNIQI |
| 19714 | IHDTLGAVA |
| 19715 | KCEVLEVQD |
| 19716 | KEVYLSTAA |
| 19717 | KHVDLSNIP |
| 19718 | LGNQLAYGG |
| 19719 | LHYPLVNDG |
| 19720 | LMDHLCTPN |
| 19721 | LRGCLHLEH |
| 19722 | LTNFLQNDN |
| 19723 | MFLPLHYKD |
| 19724 | MHELLQSVH |
| 19725 | MIAQLHTWA |
| 19726 | MNRWLLTDG |
| 19727 | MPQSLRVCS |
| 19728 | MYEWLAHCW |
| 19729 | NANLLTEQY |
| 19730 | NEMRLECSY |
| 19731 | NHKWLKCSL |
| 19732 | NHVSLNCDP |
| 19733 | NISWLHSFH |
| 19734 | NTCYLYAFF |
| 19735 | PDPVLSIKD |
| 19736 | PHCTLSFIG |
| 19737 | PRKSLWMEP |
| 19738 | PWAVLNQEP |
| 19739 | PYEQLGTTI |
| 19740 | QFGSLVNSW |
| 19741 | QGYKLDNQM |
| 19742 | QKEYLMNSG |
| 19743 | QMWTLPSHN |
| 19744 | QPMVLNQDC |
| 19745 | QQHRLTFMP |
| 19746 | QSQSLTEEA |
| 19747 | QTNALLNGE |
| 19748 | QWNGLNSCL |
| 19749 | RCLDLVNEP |
| 19750 | SADQLHQFA |
| 19751 | SAEDLYYHY |
| 19752 | SCVCLEAHQ |
| 19753 | SDWPLMRYQ |
| 19754 | SEVGLPCYQ |
| 19755 | SFNQLICGL |
| 19756 | SHDDLRMQC |
| 19757 | SPGILQMIG |
| 19758 | SREILDVVA |
| 19759 | SSQHLWHRD |
| 19760 | STVTLHTHQ |
| 19761 | SYDGLDQVA |
| 19762 | SYEILGDWC |
| 19763 | TCLNLSWYG |
| 19764 | TFGFLSDPQ |
| 19765 | THEHLANHN |
| 19766 | TMEPLGWSF |
| 19767 | TSEVLDHFC |
| 19768 | TYMQLSKYC |
| 19769 | VFHTLHTKG |
| 19770 | VGMPLRKEE |
| 19771 | VGNCLHNYW |
| 19772 | VIYQLGEMV |
| 19773 | VREFLNGLN |
| 19774 | WCWQLHDQF |
| 19775 | WDDLLSFWC |
| 19776 | WTLMLHNFM |
| 19777 | NMPSLRTRI |
| 19778 | ASFFLNPLE |
| 19779 | ATMNLSWGL |
| 19780 | FQDPKDTVG |
| 19781 | FTNSDDDVS |
| 19782 | GCEACDFMG |
| 19783 | GDNKIDMHN |
| 19784 | GHMWTDSMA |
| 19785 | GIHVTDLQE |
| 19786 | GMQWPDSME |
| 19787 | GNDIWDFMA |
| 19788 | GQNVMDYHA |
| 19789 | GSSVDDGVS |
| 19790 | GTECIDWTA |
| 19791 | GTNAKDGTP |
| 19792 | GWDRHDGFV |
| 19793 | HDKDFDWAN |
| 19794 | HHDTSDMSC |
| 19795 | HHGPMDWGA |
| 19796 | HIMCYDEWY |
| 19797 | HLYIKDMTD |
| 19798 | HSMVHDCFN |
| 19799 | HSWTRDRLE |
| 19800 | HWVMGDVKL |
| 19801 | HYDQRDEHQ |
| 19802 | HYHQSDWMH |
| 19803 | IAIPEDKQY |
| 19804 | IASLEDMNG |
| 19805 | IEMVRDCGY |
| 19806 | IHLKSDYVW |
| 19807 | IKDWVDGIQ |
| 19808 | IQQTIDMVA |
| 19809 | ITHMIDQDN |
| 19810 | ITWPEDGSI |
| 19811 | KDYASDIHK |
| 19812 | KEFRMDSGS |
| 19813 | KHHTSDTAV |
| 19814 | KIYDHDAWT |
| 19815 | KSLCYDDWM |
| 19816 | KWVNVDDMR |
| 19817 | LADSDDPMD |
| 19818 | LIASRDEAH |
| 19819 | LINMWDFWP |
| 19820 | LQHFIDEGA |
| 19821 | LQKVCDVYT |
| 19822 | LTAKPDCCN |
| 19823 | LTVVNDTVF |
| 19824 | LWLARDLMC |
| 19825 | MASFTDHPQ |
| 19826 | MEVAQDVIN |
| 19827 | MHCAVDAQC |
| 19828 | MHRHMDHEG |
| 19829 | MNCQQDCVR |
| 19830 | MQHVRDQFN |
| 19831 | MSHTTDGDE |
| 19832 | MTGYWDVEY |
| 19833 | MTIPIDEMA |
| 19834 | MTVHRDAWY |
| 19835 | MTYKWDCHV |
| 19836 | MWNIIDRHN |
| 19837 | NAPHADTPS |
| 19838 | NCRHCDPNC |
| 19839 | NCSYHDASP |
| 19840 | NDGKDDTYQ |
| 19841 | NEDMWDYNH |
| 19842 | NFRTFDHSE |
| 19843 | NHLTRDQWG |
| 19844 | NMLNADAMY |
| 19845 | NNSVCDKMS |
| 19846 | NQVGRDVMD |
| 19847 | NSDTGDMNP |
| 19848 | NTMHTDTME |
| 19849 | NTMLTDTGS |
| 19850 | QCMHDDWSS |
| 19851 | QCMWPDIAG |
| 19852 | QEKLQDMYG |
| 19853 | QFMDFDFHY |
| 19854 | QLMPRDGLC |
| 19855 | QQEHADWWR |
| 19856 | QSENMDYWD |
| 19857 | QTFDADGMH |
| 19858 | QTQPHDRHH |
| 19859 | QYHHIDYGK |
| 19860 | QYQNDDRAS |
| 19861 | QYYRADGAK |
| 19862 | RQVMTDRIN |
| 19863 | RSEFADHWW |
| 19864 | SAEYWDMQD |
| 19865 | SAIWGDNSM |
| 19866 | SDETRDKHD |
| 19867 | SFSFCDQRT |
| 19868 | SIEDFDEDK |
| 19869 | SMEPYDTFF |
| 19870 | SNRWQDTYY |
| 19871 | SPEIWDMGA |
| 19872 | SSSQCDVYE |
| 19873 | STLQSDPFE |
| 19874 | STMFKDHFA |
| 19875 | TARNHDHTA |
| 19876 | TCHDVDFTM |
| 19877 | TGGLWDWFE |
| 19878 | THVGIDNIP |
| 19879 | TIRYFDGCG |
| 19880 | TLHDNDSAC |
| 19881 | TMEVSDQHN |
| 19882 | TMGIYDMNE |
| 19883 | TMRTSDVYE |
| 19884 | TNYQVDWDS |
| 19885 | TQMMYDCMQ |
| 19886 | TSKSYDGQC |
| 19887 | TSSVVDLNA |
| 19888 | VCALYDEQS |
| 19889 | VCCYIDCVQ |
| 19890 | VCSMNDSCF |
| 19891 | VDCIIDPQA |
| 19892 | VEKGQDMDN |
| 19893 | VGDQPDAGP |
| 19894 | VGVQDDFMY |
| 19895 | VHYNVDQCA |
| 19896 | VMVKRDVAR |
| 19897 | VNGVTDMDD |
| 19898 | VPPSADDPH |
| 19899 | VPWVSDWRC |
| 19900 | VQLIKDWDQ |
| 19901 | VSMRCDADY |
| 19902 | VSQPFDPYP |
| 19903 | VSRIDDAAC |
| 19904 | VTHWCDHSY |
| 19905 | WDSCSDSWM |
| 19906 | WFRMRDCCG |
| 19907 | WLVIKDVNL |
| 19908 | WMMQVDGYP |
| 19909 | WRKMEDNCM |
| 19910 | YEQGVDRTF |
| 19911 | YGCKSDEGF |
| 19912 | YQIQFDQII |
| 19913 | YSEMGDFYG |
| 19914 | YSSFTDQNV |
| 19915 | YTFYKDCCC |
| 19916 | DNILRDVMA |
| 19917 | QISVYDTKY |
| 19918 | STALMDANM |
| 19919 | MSGPNDTEF |
| 19920 | GIWVRDTYN |
| 19921 | DHLSNDAEM |
| 19922 | ATGLTDHQW |
| 19923 | QSRCVDNTV |
| 19924 | GCAVIEVSA |
| 19925 | GKWYNGVEL |
| 19926 | GRNNAWVHV |
| 19927 | GSMMFGVAQ |
| 19928 | GTSWVLVQY |
| 19929 | GTYLHVVAD |
| 19930 | GWSDMTVVF |
| 19931 | HHSIKNVCH |
| 19932 | IDFITTVHY |
| 19933 | IDHHFVVIE |
| 19934 | IHEHHPVIF |
| 19935 | ILGVHNVEA |
| 19936 | IPHHIFVQD |
| 19937 | IYVMPVVER |
| 19938 | KDENNPVAP |
| 19939 | KEDMTSVGQ |
| 19940 | KEVTSQVSY |
| 19941 | KFVHTQVEC |
| 19942 | KNGCITVQP |
| 19943 | KQFNHMVTH |
| 19944 | KSVDKYVVD |
| 19945 | LCHDKIVHV |
| 19946 | LEVVQVVGG |
| 19947 | MLPTCMVVN |
| 19948 | MMASKFVGR |
| 19949 | MRMPDGVNT |
| 19950 | MWEATCVRS |
| 19951 | NAELYPVEW |
| 19952 | NGEQAFVMV |
| 19953 | NHCYVEVGG |
| 19954 | NPQYGCVME |
| 19955 | NYHDYQVCY |
| 19956 | PGIFGCVGW |
| 19957 | PSNNKCVWC |
| 19958 | QKHCQCVSV |
| 19959 | QTGTGWVGN |
| 19960 | QWGTTNVIT |
| 19961 | RADNTNVSW |
| 19962 | RINHYHVWV |
| 19963 | RSCGEVVFT |
| 19964 | RWDGQEVWK |
| 19965 | SGYYPWVLT |
| 19966 | SHNFKQVMR |
| 19967 | SLHPVFVNS |
| 19968 | SQFWAHVCP |
| 19969 | SRVNCFVIV |
| 19970 | STILMPVHI |
| 19971 | TAVAWCVSS |
| 19972 | TAVLREVFM |
| 19973 | TCYTCCVWY |
| 19974 | THCTKVVDT |
| 19975 | TIEVMSVWN |
| 19976 | TMRTQCVAL |
| 19977 | TPLTCMVCA |
| 19978 | TQFHCPVVK |
| 19979 | TQSSQTVAY |
| 19980 | TSQQGIVSL |
| 19981 | TYADMWVGC |
| 19982 | TYHTVSVCD |
| 19983 | VGSWVEVQN |
| 19984 | VKQYWHVEE |
| 19985 | VMETHWVWA |
| 19986 | VNPTNKVHG |
| 19987 | VTELRYVSH |
| 19988 | YAEVNPVIG |
| 19989 | YHQWPFVDM |
| 19990 | YKKAWMVMQ |
| 19991 | YNPSEEVDG |
| 19992 | YQGTMIVGS |
| 19993 | YSCKERVGV |
| 19994 | SFSYDVVMN |
| 19995 | MFSHGHVMF |
| 19996 | DCVLTMVDK |
| 19997 | QWFTKGVGE |
| 19998 | FPGVFKNWV |
| 19999 | GASWYCSWM |
| 20000 | GFQWDAHWN |
| 20001 | GFVSYHSWD |
| 20002 | GIAVRSAWF |
| 20003 | GISDDAYWW |
| 20004 | GMMLMPSWC |
| 20005 | GNAACNAWP |
| 20006 | GTPRDEQWY |
| 20007 | GYNTANSWQ |
| 20008 | HLDWFNMWQ |
| 20009 | ICEHRIFWD |
| 20010 | IDQNFCMWG |
| 20011 | IHHGCKMWI |
| 20012 | IPMLCRIWM |
| 20013 | IREPMPAWA |
| 20014 | ISAIAGMWG |
| 20015 | ITAATTTWK |
| 20016 | IWYVRCWWA |
| 20017 | KKKNCILWR |
| 20018 | LADSQVFWT |
| 20019 | LPDKAAHWS |
| 20020 | LQPRTGPWK |
| 20021 | LRPHIHAWE |
| 20022 | LSENITGWW |
| 20023 | LSPWNFGWD |
| 20024 | LTVPHCSWQ |
| 20025 | MASCKVAWS |
| 20026 | MCDWREKWT |
| 20027 | MFVCRQFWK |
| 20028 | MGAANYSWY |
| 20029 | MGSIWLEWH |
| 20030 | MIESVSHWD |
| 20031 | MIFPIYSWA |
| 20032 | MPMCHCGWN |
| 20033 | MREVHCTWM |
| 20034 | MTAADVCWG |
| 20035 | MTLDFSTWG |
| 20036 | NGMQICCWY |
| 20037 | NHVIYHDWE |
| 20038 | NIDHQFHWW |
| 20039 | NLKWHKRWT |
| 20040 | NMIQDPYWN |
| 20041 | NMVNAGGWS |
| 20042 | NQMFWNIWG |
| 20043 | NSGPFYCWT |
| 20044 | PLRWDESWG |
| 20045 | QANWTMMWD |
| 20046 | QIKFVEYWG |
| 20047 | QNRYSELWA |
| 20048 | QSYWFQQWA |
| 20049 | QTSSCEEWG |
| 20050 | QYKQVLDWY |
| 20051 | RIDAQQQWF |
| 20052 | RSADSWEWT |
| 20053 | RTIAWEKWQ |
| 20054 | RWDAGRTWF |
| 20055 | SEDQFHHWC |
| 20056 | SIDNKWPWR |
| 20057 | SKFVRYDWG |
| 20058 | SLCADPDWP |
| 20059 | SMWGPRAWT |
| 20060 | SPYNECHWL |
| 20061 | SQGITGRWV |
| 20062 | STEDCVSWY |
| 20063 | TADVEPPWN |
| 20064 | TALRQMAWH |
| 20065 | TFEPYSWWL |
| 20066 | THGCMLTWT |
| 20067 | THHQMFEWT |
| 20068 | TIASFMQWD |
| 20069 | TKAMIWHWG |
| 20070 | TMRHWCRWP |
| 20071 | TRMMMQEWP |
| 20072 | VAVKEAQWH |
| 20073 | VCHWSLCWL |
| 20074 | VFHSTHWWY |
| 20075 | VSEYYWEWN |
| 20076 | VTSTILHWQ |
| 20077 | WILNAPEWQ |
| 20078 | WQCAMVMWI |
| 20079 | YPWCYMDWV |
| 20080 | YTHDTGLWM |
| 20081 | LFRTIPIWQ |
| 20082 | YWCPSQWWR |
| 20083 | KMAGIGCWY |
| 20084 | MYDTMGAWC |
| 20085 | FTPMNRDRI |
| 20086 | GHYWAHEAI |
| 20087 | GIFTTNFEI |
| 20088 | GMDVNHCGI |
| 20089 | GSCITMGSI |
| 20090 | HSDLMVCQI |
| 20091 | HYCYGEFGI |
| 20092 | IACTSLPII |
| 20093 | IAENFQGCI |
| 20094 | ICHAKATNI |
| 20095 | ILKLPWLNI |
| 20096 | MNMTYYTEI |
| 20097 | MSEAIGHKI |
| 20098 | MTDLARTYI |
| 20099 | NCPSDQPSI |
| 20100 | NEVHYEMAI |
| 20101 | NRSKASGDI |
| 20102 | NSWFKPEFI |
| 20103 | NWYKDVIRI |
| 20104 | PAESCNQKI |
| 20105 | RYPHEVNGI |
| 20106 | SCQAVWCGI |
| 20107 | SRETFTKHI |
| 20108 | TCADGMDHI |
| 20109 | TKECSHEVI |
| 20110 | TSRFEMHTI |
| 20111 | VTAMFGQEI |
| 20112 | VTNPKQEAI |
| 20113 | WMGAMVHDI |
| 20114 | YGPKFWIAI |
| 20115 | YMDMRNLVI |
| 20116 | WPNIKHQPI |
| 20117 | YSDPKNMSI |

### Example 17

AAV5 Variants with Tissue Tropism in Skin

This example describes engineered AAV5 variants with tissue tropism in skin that were discovered using the methods and systems described in EXAMPLE 1-EXAMPLE 2.

CNS, liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive skin tropism. The results are shown in FIG. 18J which shows the positional frequency of each amino acid in the 581 to 589 region of AAV5 VP1 in skin tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in skin over the frequency of that given amino acid residue occurring at the specified position in variants identified in all other harvested tissues was analyzed to identify a set of sequence rules for capsids that preferentially target skin tissue. With reference to TABLE 33 below, and SEQ ID NO: 2 (AAV5 VP1), the following amino acids can, thus, be independently mutated, in any combination, at any one or more positions Xaa1-Xaa9, to provide a VP1 capsid with enhanced skin tropism as compared wildtype AAV5 VP1 capsid (SEQ ID NO: 1), where skin tropism here refers to properties that are preferred for skin transduction over properties that are preferred for transduction of all other harvested tissues.

TABLE 33
Skin Tropism Rules

Xaa1 is selected from A, C, K, Q, R, or T
Xaa1 is selected from C, K, or R
Xaa1 is C
Xaa2 is selected from A, C, I, S, T, or V
Xaa2 is selected from A, S, T, or V
Xaa2 is V
Xaa3 is selected from A, C, F, G, M, Q, S, or V
Xaa3 is selected from A, C, F, M, or Q
Xaa3 is C
Xaa4 is selected from C, K, L, P, R, or W
Xaa4 is selected from L, P, or R
Xaa4 is R
Xaa5 is selected from F, H, I, M, V, or Y
Xaa5 is selected from M, V, or Y
Xaa5 is Y
Xaa6 is selected from F, H, I, M, N, Q, or S
Xaa6 is selected from M, N, or Q
Xaa6 is N
Xaa7 is selected from A, H, K, M, N, R, or V
Xaa7 is A, H, K, or R
Xaa7 is K
Xaa8 is selected from A, F, G, H, S, or Y
Xaa8 is selected from A, F, or S
Xaa8 is S
Xaa9 is selected from A, E, G, P, Q, R, or S
Xaa9 is selected from A, Q, or S
Xaa9 is A

TABLE 34 below provides sequences of the 581 to 589 region for variant AAV VP1 capsid polypeptides that were found in skin tissue and comport to one or more of the rules provided in TABLE 33. The present disclosure, thus, provides for rAAVs composed of engineered AAV5 VP1 capsid polypeptides having a SEQ ID NO: 2, wherein the Xaa1 to Xaa9 region is any one of SEQ ID NO: 31991-SEQ ID NO: 32990, as disclosed in TABLE 34. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 34
Sequences of the 581 to 589 Region in AAV5
VP1 Capsid Polypeptide that Drive Skin
Tissue Tropism

| SEQ | |
|---|---|
| ID | 581-589 |
| NO | Sequence |
| 31991 | CAFAYTIQS |
| 31992 | CAFMTHCYA |
| 31993 | CAGPWCHHE |
| 31994 | CAIVCFDSF |
| 31995 | CAMWHQKGM |
| 31996 | CANCTWVAE |
| 31997 | CASWYATDR |
| 31998 | CAVWANWLC |
| 31999 | CAWLATDFK |
| 32000 | CCDHVQSSW |
| 32001 | CCMPMFRNA |
| 32002 | CFAAYRMQG |
| 32003 | CFAKWWGYE |
| 32004 | CFALKINYS |
| 32005 | CFCIMVNGY |
| 32006 | CFCQRPLWP |
| 32007 | CFLRNNAEY |
| 32008 | CFRYAEIGP |
| 32009 | CGAIHSKRD |
| 32010 | CHARGYIMP |
| 32011 | CHCMPLVIF |
| 32012 | CITHTLKDG |
| 32013 | CKVMKMEGE |
| 32014 | CLVWERRFS |
| 32015 | CMSMNHWMR |
| 32016 | CNAKMTDLA |
| 32017 | CPNIAPIPG |
| 32018 | CQLTVHSFG |
| 32019 | CQSSYSMYA |
| 32020 | CQTRFYVMP |
| 32021 | CRGTIAVGI |
| 32022 | CRMFKIDAI |
| 32023 | CRSRYEAQC |
| 32024 | CSIKCAFGC |
| 32025 | CSITHQRGC |
| 32026 | CSQKIAYAK |
| 32027 | CSSPQYFSM |
| 32028 | CSTYYCNGL |
| 32029 | CSVKCAFGC |
| 32030 | CSYPWDNLG |
| 32031 | CTATYTIRD |
| 32032 | CTCQYGWTV |
| 32033 | CTFYYFKHH |
| 32034 | CTKGWMVIG |
| 32035 | CTLRTWSGM |
| 32036 | CTMFYEQSK |
| 32037 | CTMKYQEHK |
| 32038 | CTMQALRQA |
| 32039 | CTTKFEKCW |
| 32040 | CTTTFACPT |
| 32041 | CTWMAFTAP |
| 32042 | CTWPRCDSF |
| 32043 | CTWVTHGQQ |
| 32044 | CVCGKMGHG |
| 32045 | CVDPDDYGY |
| 32046 | CVEIMYMNE |
| 32047 | CVEYWGQCN |
| 32048 | CVGSRPEAA |
| 32049 | CVIWDQAQN |
| 32050 | CVLTQQFNA |
| 32051 | CVMHSTDHH |
| 32052 | CVQRKDRGT |
| 32053 | CVQRKMQAV |
| 32054 | CVQTKINCG |
| 32055 | CVQYNKCRS |
| 32056 | CVRYYPRPA |
| 32057 | CVTMALMDQ |
| 32058 | CVTTSNYTM |
| 32059 | CVTVTYDGR |
| 32060 | CVVHKQKEY |
| 32061 | CVVHTMRWC |
| 32062 | CVVKHLFGH |
| 32063 | CWSYGQTAA |
| 32064 | CAAQHCAQV |
| 32065 | CAAQHYAQV |
| 32066 | CAGIQATGD |
| 32067 | CAIHTRSHS |
| 32068 | CAIMFSAGP |
| 32069 | CAIMLQHMT |
| 32070 | CALPVLVAM |
| 32071 | CALPYAWSR |
| 32072 | CALSMYSQE |
| 32073 | CALTYAWSR |
| 32074 | CAVYFGKCA |
| 32075 | CCRYYVNVE |
| 32076 | CEKLTQNVD |
| 32077 | CEYFKRHPD |
| 32078 | CFHQYHQAH |
| 32079 | CFHRYHQAH |
| 32080 | CFKIWDLGN |
| 32081 | CFMGLSFHW |
| 32082 | CFVEPKTSC |
| 32083 | CFVWEFNYP |
| 32084 | CGDCYGWCQ |
| 32085 | CGGFCTALE |
| 32086 | CHAQIQHGG |
| 32087 | CHAQSIMGP |
| 32088 | CHFMINAGR |
| 32089 | CHHFATESE |
| 32090 | CHHTTNTSQ |
| 32091 | CHVPLNNCD |
| 32092 | CIKACYDHE |
| 32093 | CIKSYNVMM |
| 32094 | CILHKHSFP |
| 32095 | CIMLNARVG |
| 32096 | CIQLNKMGP |
| 32097 | CIVTVPQQA |
| 32098 | CIWMQHFSL |
| 32099 | CKFDMPSFF |
| 32100 | CKNFHWMND |
| 32101 | CKQQINDAY |
| 32102 | CKSHFDQVE |
| 32103 | CKYDITKMA |
| 32104 | CLELVKVYE |
| 32105 | CLNTYHYQG |
| 32106 | CLSSSYNSY |
| 32107 | CMHLVNMNY |
| 32108 | CMQFIRIGS |
| 32109 | CMQFIRVGS |
| 32110 | CMVHNHKGF |
| 32111 | CMYWLTNQE |
| 32112 | CNCHIRHTG |
| 32113 | CNFQTNVHL |
| 32114 | CNTQVYGNY |
| 32115 | CNVDRYNRP |
| 32116 | CPKPFLIQE |
| 32117 | CQELRTQYA |
| 32118 | CQEVYAHMS |
| 32119 | CQSVHYRAP |
| 32120 | CRAAHSVCA |
| 32121 | CRATHSVCA |
| 32122 | CSGCYMKMP |
| 32123 | CSGTMLSQP |
| 32124 | CSHHVDRVM |
| 32125 | CSHQKLMES |
| 32126 | CSMIQNSFQ |
| 32127 | CSTDHALVE |
| 32128 | CSTMVRSQT |
| 32129 | CSTWLNQHM |
| 32130 | CTCNLPYAI |
| 32131 | CTEMLQRKG |
| 32132 | CTGQLMHKE |
| 32133 | CTHGFLTGP |
| 32134 | CTHKVNSWI |
| 32135 | CTIFNTVDQ |
| 32136 | CTIRANWHF |
| 32137 | CTMLANHYQ |
| 32138 | CTPGFWSER |
| 32139 | CTQFTKHQE |
| 32140 | CTTAWQGAW |
| 32141 | CTTVKMWHT |
| 32142 | CTTYRNLLS |
| 32143 | CTYFSMMDR |
| 32144 | CTYMQMAYG |
| 32145 | CVFQCYRSP |
| 32146 | CVFVQQNFQ |
| 32147 | CVHAASLMP |
| 32148 | CVHLGHWQW |
| 32149 | CVLPVLVAM |
| 32150 | CVLWQVHSE |
| 32151 | CVNQHSLAM |
| 32152 | CVPISAAPV |
| 32153 | CVRLIDWCC |
| 32154 | CVVMKFSEH |
| 32155 | CVYWQQNHE |
| 32156 | CWGPKFVDF |
| 32157 | CWMPQQVAT |
| 32158 | CYPVYKDWS |
| 32159 | CYTIKQMDI |
| 32160 | CANHLNQTI |
| 32161 | CWAVTSMSS |
| 32162 | CTCWYNRGD |
| 32163 | CTHPVHDNE |
| 32164 | CYQVTIQHN |
| 32165 | CTLMHSHTR |
| 32166 | CIIGRQEWL |
| 32167 | CSRNRCWSG |
| 32168 | CSIQINNCR |
| 32169 | CGHRCEMNM |
| 32170 | CNSYNKPWM |
| 32171 | CMHLSCDWT |
| 32172 | CYAEEPVWP |
| 32173 | CNSWSTGSL |
| 32174 | CMNHRWSTT |
| 32175 | CWHFFGADR |
| 32176 | CARLEGRGN |
| 32177 | CMEAQGECY |
| 32178 | CTKPIPCWM |
| 32179 | CDCLHHCCM |
| 32180 | CIWSSPDQP |
| 32181 | CADPFSIRG |
| 32182 | CSMPQQCME |
| 32183 | CTMCHDCSP |
| 32184 | CTEISWGGV |
| 32185 | CVGYKSNTP |
| 32186 | CSESYRGPD |
| 32187 | CWECCTDSG |
| 32188 | EVMPVNRKH |
| 32189 | EVTRFSEPG |
| 32190 | GVAYEEDYY |
| 32191 | HVFSHWNCN |
| 32192 | IVADLNMFY |
| 32193 | IVGMQMTGE |
| 32194 | IVNSFQKMS |
| 32195 | IVTTVSRTA |
| 32196 | KVEQYMQCH |
| 32197 | KVTMESFDL |
| 32198 | LVDHMSTGC |
| 32199 | LVGPTDQLP |
| 32200 | MVGQLMKES |
| 32201 | NVFYVGEWP |
| 32202 | NVMLGAGTI |
| 32203 | NVQKQQTDA |
| 32204 | NVQRLCCLQ |
| 32205 | PVSLFGLIH |
| 32206 | QVCAMVQCR |
| 32207 | SVKHKEMVG |
| 32208 | SVNYCPRGP |
| 32209 | SVTCLYTSF |
| 32210 | TVDRCFQYA |
| 32211 | TVFYCTKMH |
| 32212 | TVGIEWLNW |
| 32213 | TVGSWAPGS |
| 32214 | TVMRALVYN |
| 32215 | VVVDLWWHT |
| 32216 | WVGCTNHGQ |
| 32217 | YVVDLKNFG |
| 32218 | AVETEFRER |
| 32219 | AVNCTIYGP |
| 32220 | AVNTQDDLF |
| 32221 | AVRNVEVSR |
| 32222 | AVSTWAMQS |
| 32223 | DVCKVMTRV |
| 32224 | DVGSWLNKC |
| 32225 | DVNHTKEFN |
| 32226 | DVRVDMNHA |
| 32227 | DVYNNVKLG |
| 32228 | EVCRIERYM |
| 32229 | EVDIVINNY |
| 32230 | EVIFMNCPL |
| 32231 | EVNEEEVTY |
| 32232 | EVWWEESKS |
| 32233 | EVYPCMSYG |
| 32234 | FVHCTNALF |
| 32235 | FVTCNQYAP |
| 32236 | GVFYTMASY |
| 32237 | GVKCINSPV |
| 32238 | GVMNHIAWT |
| 32239 | HVCMLEFGT |
| 32240 | HVMQGAPTD |
| 32241 | HVVMKHGES |
| 32242 | IVAKISWSE |
| 32243 | IVCPAGQYH |
| 32244 | IVEPSGHFL |
| 32245 | IVVSMDQWL |
| 32246 | LVGPEGAWN |
| 32247 | LVMQLYCAG |
| 32248 | LVMRMCADG |
| 32249 | LVTQDMKRY |
| 32250 | MVTQPCAWQ |
| 32251 | NVERIQSLG |
| 32252 | NVFIATAQT |
| 32253 | NVGIFWYCG |
| 32254 | PVAWCDKLG |
| 32255 | QVAYAFEAN |
| 32256 | QVHYMMCYE |
| 32257 | QVLSQWFDN |
| 32258 | QVVQKAMAE |
| 32259 | SVMTTFCHN |
| 32260 | TVGITMLPM |
| 32261 | TVHEIMENN |
| 32262 | VVMPKESNE |
| 32263 | YVCGSGMEQ |
| 32264 | YVDKRSTME |
| 32265 | AVETGESAM |
| 32266 | AVATRAMYS |
| 32267 | AVSCGQQRE |
| 32268 | DVAIMQGKG |
| 32269 | DVLMDKRHC |
| 32270 | DVNEPIQCQ |
| 32271 | DVNLDPDVC |
| 32272 | DVYKSPCTT |
| 32273 | EVGINSEGL |
| 32274 | EVGLCQSGR |
| 32275 | EVLKSFMCQ |
| 32276 | EVMFMKKHL |
| 32277 | EVQVTKAQE |
| 32278 | EVSHRVSAA |
| 32279 | EVTQLSCQV |
| 32280 | EVVCSMYYG |
| 32281 | FVASVPDMS |
| 32282 | FVDFNHKQW |
| 32283 | FVDTYEAPG |
| 32284 | FVEPYTDHQ |
| 32285 | FVTMLHWQS |
| 32286 | GVAVIRKMQ |
| 32287 | GVEEFNAQD |
| 32288 | GVFPNMFAP |
| 32289 | GVGMKCYSH |
| 32290 | GVLPVQQGF |
| 32291 | GVQPWESCT |
| 32292 | GVSAYCTSA |
| 32293 | GVWIMRSKR |
| 32294 | GVYYYTKQP |
| 32295 | HVAFQWSGQ |
| 32296 | HVALQNDWG |
| 32297 | IVAHQCQYC |
| 32298 | IVAKYGMTS |
| 32299 | IVDQRSVWV |
| 32300 | IVQPCQSNW |
| 32301 | IVRSALTNT |
| 32302 | KVCFKEDAF |
| 32303 | LVNEMWYHS |
| 32304 | LVQHSVVWM |
| 32305 | LVTGMMSMF |
| 32306 | MVAQHGISL |
| 32307 | MVAQYGISL |
| 32308 | MVEHNRLAN |
| 32309 | MVGMLYKTK |
| 32310 | MVVYRENMG |
| 32311 | MVWEVKFND |
| 32312 | MVWEVKFNG |
| 32313 | NVCAMPMTH |
| 32314 | NVCTMPMTH |
| 32315 | NVMPSPMDR |
| 32316 | NVQAYERAN |
| 32317 | NVQRHHIFQ |
| 32318 | NVVQSFTAK |
| 32319 | PVQHIYWGL |
| 32320 | QVEVDHCWA |
| 32321 | QVGVCDQCQ |
| 32322 | QVMENYELK |
| 32323 | QVMQNQCYT |
| 32324 | QVTMQPYSK |
| 32325 | QVTRDGKGG |
| 32326 | QVVQYLTNH |
| 32327 | RVDVTNHRY |
| 32328 | RVEPTTVPD |
| 32329 | RVQSQWDGC |
| 32330 | SVCPTRYAD |
| 32331 | SVVCTTEFS |
| 32332 | SVWASTVFH |
| 32333 | TVARGPVPG |
| 32334 | TVGNSKMQN |
| 32335 | TVHWDITGC |
| 32336 | TVMSAFDWG |
| 32337 | TVTTKVRDS |
| 32338 | VVCMHHCDM |
| 32339 | VVGQKMTQT |
| 32340 | VVQQIRDFH |
| 32341 | WVEWPQMGS |
| 32342 | WVVCRCAWE |
| 32343 | YVPDRDGCD |
| 32344 | YVQLKQEWY |
| 32345 | YVVGMGKFC |
| 32346 | PVLHIYWGL |
| 32347 | YVHPWFGVS |
| 32348 | KVNACGNLT |
| 32349 | PVNLAQRDK |
| 32350 | EVAGCATAV |
| 32351 | SVKLAIDQK |
| 32352 | LVGKYIDSM |
| 32353 | DVMQNQCKS |
| 32354 | HVVPCCPFH |
| 32355 | DVISRNGRC |
| 32356 | LVDCSNTCQ |
| 32357 | AVEDYWRYF |
| 32358 | GVAQYLHHC |
| 32359 | QVEDGYWSY |
| 32360 | MVFLEQHGR |
| 32361 | LVQKWMRTC |
| 32362 | VVISTKHTH |
| 32363 | DVDVVALPM |
| 32364 | RVTIMFTGT |
| 32365 | AVNACSNSH |
| 32366 | TVWIQSYSN |
| 32367 | PVNESVHIC |
| 32368 | SVRTCHQDE |
| 32369 | DVTQHTSCT |
| 32370 | PVEMKFAVQ |
| 32371 | GVWMQGFHP |
| 32372 | QVHMQRYYG |
| 32373 | KVAHEMMSA |
| 32374 | DVCKATTTE |
| 32375 | AVHNVKDVH |
| 32376 | NVYATPVVC |
| 32377 | NVMGTQAKE |
| 32378 | DVKAFMGGV |
| 32379 | RVNGSTCRH |
| 32380 | SVYMMTEGW |
| 32381 | HVTKLMTSH |
| 32382 | ELCTNWTAN |
| 32383 | EPCQSHEFY |
| 32384 | ETCPYFDFD |
| 32385 | GGCVYWQMD |
| 32386 | GTCTDMGIC |
| 32387 | HTCHFGGFV |
| 32388 | ISCQSMCCE |
| 32389 | ITCQKMAMN |
| 32390 | KDCFNGECE |
| 32391 | KPCMFWMSR |
| 32392 | KTCAVNVYL |
| 32393 | LACQIQWMT |
| 32394 | MTCEYRTSM |
| 32395 | MWCDYGTGW |
| 32396 | QACFIAGQQ |
| 32397 | QDCCKQSVA |
| 32398 | QGCKQMGSM |
| 32399 | SSCYHSTGA |
| 32400 | STCTIRGNP |
| 32401 | TACTMFECQ |
| 32402 | VACWYTKHS |
| 32403 | VICQVQGRA |
| 32404 | VYCLTHAGP |
| 32405 | YICRLWTQC |
| 32406 | YTCVMEHMH |
| 32407 | AMCPTHWHQ |
| 32408 | DRCNFNCSE |
| 32409 | DSCPQIFRD |
| 32410 | EWCLADGMT |
| 32411 | GACKFNGHK |
| 32412 | GRCQYFQMA |
| 32413 | GWCWYMRMG |
| 32414 | IGCKVQSTN |
| 32415 | ISCKAHWQG |
| 32416 | ITCVDNKMC |
| 32417 | KSCEWMLQQ |
| 32418 | KTCEWGQDK |
| 32419 | LCCPFDTDA |
| 32420 | LKCMNHSAW |
| 32421 | LKCVFMRAE |
| 32422 | LSCLATYQS |
| 32423 | MLCEPEGAR |
| 32424 | NYCTYHSTH |
| 32425 | QSCATIIRE |
| 32426 | QSCLQQIQA |
| 32427 | RSCTKEDYW |
| 32428 | RTCWFMGVG |
| 32429 | SGCPQRVAT |
| 32430 | SHCWYWYWN |
| 32431 | SNCPYHTFY |
| 32432 | SNCVTDATA |
| 32433 | TNCAMQMYY |
| 32434 | TTCRTWEAF |
| 32435 | VACRENMLT |
| 32436 | VLCHEADGP |
| 32437 | VYCPCDGPD |
| 32438 | YACHMGHYD |
| 32439 | YPCLMEKEY |
| 32440 | YSCMVNTHQ |
| 32441 | YSCSQAMHG |
| 32442 | AACENKVCF |
| 32443 | AMCLFETMQ |
| 32444 | AQCKLTTVE |
| 32445 | ATCWQPAAK |
| 32446 | DNCKIMFMC |
| 32447 | DPCPNMIWT |
| 32448 | DWCTYRAQD |
| 32449 | EACYMYHNT |
| 32450 | EGCYFWKQV |
| 32451 | EICTASPQA |
| 32452 | ESCLTGGNT |
| 32453 | ETCQKYVAK |
| 32454 | FICQIKSAS |
| 32455 | HACPKHYCR |
| 32456 | HCCFKNGDM |
| 32457 | HMCPFMDQA |
| 32458 | IACVSWFTC |
| 32459 | ILCWHHYSV |
| 32460 | ISCRTQSAC |
| 32461 | ITCITWVNG |
| 32462 | KACDYFMHG |
| 32463 | KHCHSMQFE |
| 32464 | KSCLYFDAY |
| 32465 | MACDYFMHG |
| 32466 | MACPKNISG |
| 32467 | MACSMNVAL |
| 32468 | MMCFNYFDR |
| 32469 | NACSHQHLG |
| 32470 | NPCPNMIWT |
| 32471 | NYCRRTFHH |
| 32472 | QACRLTYWY |
| 32473 | QACWLTYWY |
| 32474 | QMCTLTKAD |
| 32475 | RACHKQLWV |
| 32476 | RPCWPWDHL |
| 32477 | RSCFEMKHI |
| 32478 | RTCNLPYAI |
| 32479 | RYCSTIYYS |
| 32480 | SACPWNFYC |
| 32481 | SGCFWRNTS |
| 32482 | SHCLYWMKH |
| 32483 | SICQTNFAV |
| 32484 | SKCGFDVAA |
| 32485 | SLCTIHGAQ |
| 32486 | TACHISHYD |
| 32487 | TACYKNTCE |
| 32488 | TACYPHSFG |
| 32489 | TICSMGQKD |
| 32490 | TICYMSTSS |
| 32491 | TYCRSGDVQ |
| 32492 | VMCTWQACR |
| 32493 | WHCNFHWHL |
| 32494 | WYCLTQRSD |
| 32495 | WYCMCIIRE |
| 32496 | YACATQQHL |
| 32497 | YACPKHYCR |
| 32498 | YHCKFNWLG |
| 32499 | YHCQNYAAS |
| 32500 | YKCAHDPTG |
| 32501 | IYCWMPMSP |
| 32502 | EHCNHDQGV |
| 32503 | KACMSHQGR |
| 32504 | MSCPAKIVV |
| 32505 | QNCIMLASN |
| 32506 | GTCLFKTHK |
| 32507 | KPCNPVGQE |
| 32508 | RLCDTHKCL |
| 32509 | EMCGGGLSC |
| 32510 | EACMPWAHH |
| 32511 | IMCHARMMA |
| 32512 | AQCHDMWGH |
| 32513 | ESCHLQHGE |
| 32514 | WACIIYMWN |
| 32515 | GRPRTPLGN |
| 32516 | GTIRKEKYY |
| 32517 | IPRRLQLPI |
| 32518 | ITQRDNIAM |
| 32519 | IYERHHCQA |
| 32520 | KDNRRIYKQ |
| 32521 | MALRSPFNL |
| 32522 | MCDRAIRSD |
| 32523 | QMYRLAYSR |
| 32524 | RLSRAHLPQ |
| 32525 | SIERHCRME |
| 32526 | YDWRTMAEV |
| 32527 | YSERYINAY |
| 32528 | AASRSHQSE |
| 32529 | DGVRMHKDW |
| 32530 | ETKRSDHEV |
| 32531 | LCARGHKSG |
| 32532 | MCLREIQMQ |
| 32533 | MPQRYNRET |
| 32534 | NCARGWMII |
| 32535 | TGERTHMCL |
| 32536 | VSRRSDWSE |
| 32537 | YFPRPFKMT |
| 32538 | YMDRMSECY |
| 32539 | AFDRHSDLA |
| 32540 | AGGRIGDSQ |
| 32541 | DFVRTNIGV |
| 32542 | EFVRNPCFE |
| 32543 | ESARYIDFT |
| 32544 | FAQRTTDHR |
| 32545 | FHDRPFTFT |
| 32546 | FQDREQFWY |
| 32547 | GWMRGEYNV |
| 32548 | ICVRWHCGP |
| 32549 | IRVRWHCGP |
| 32550 | KCQRMDTWK |
| 32551 | KGDRFMVDQ |
| 32552 | LAARIGEVE |
| 32553 | LIARYGKQW |
| 32554 | LMARIQNIT |
| 32555 | MADREMAAR |
| 32556 | MCIRSGFIV |
| 32557 | MEDRLAHYH |
| 32558 | NCIRNWQHA |
| 32559 | NGIRIFLFG |
| 32560 | PMSRQLYSD |
| 32561 | QGVRILTAA |
| 32562 | QWMRWLIVT |
| 32563 | TADRWCDAS |
| 32564 | TCTRKDDDM |
| 32565 | TMARFPCIS |
| 32566 | VSERAHCGE |
| 32567 | WEHRFSASM |
| 32568 | WGIRNMELR |
| 32569 | YADRFACYQ |
| 32570 | YANRNFSPE |
| 32571 | YENRGREHN |
| 32572 | AFDRHSDWA |
| 32573 | MEVRECGWD |
| 32574 | MGQRYDIQG |
| 32575 | QAVRRWWFF |
| 32576 | HHLRNVHHC |
| 32577 | KATRDPKFP |
| 32578 | NSTRNTSYY |
| 32579 | MTERYYWCQ |
| 32580 | MTQRVVHWG |
| 32581 | NNDRHQHLK |
| 32582 | NILRYLICD |
| 32583 | MPTRMQKGA |
| 32584 | DIGRFTAYY |
| 32585 | EAWRNSSSG |
| 32586 | AQARTIGLG |
| 32587 | TKGRNDHME |
| 32588 | GQFRSGAYR |
| 32589 | LNARTMRSA |
| 32590 | MMKRNRFIC |
| 32591 | EYSRMNKAS |
| 32592 | SYARKVYDC |
| 32593 | ICIRGARAD |
| 32594 | ERMCYEDSY |
| 32595 | ESGFYATWN |
| 32596 | FGGHYWPCP |
| 32597 | GHGSYLAED |
| 32598 | GNQPYGDNQ |
| 32599 | GQVGYQMHG |
| 32600 | GYHMYQKTS |
| 32601 | ITMSYAHMC |
| 32602 | IWRDYDQCY |
| 32603 | QKRDYQFMP |
| 32604 | QMNSYPMYE |
| 32605 | RADAYTQIA |
| 32606 | SCELYMARD |
| 32607 | ACSCYMHTG |
| 32608 | DISMYMFQD |
| 32609 | DMNMYLGSQ |
| 32610 | ECIWYDNYF |
| 32611 | GFYWYEMAL |
| 32612 | HYAAYEQAM |
| 32613 | IKMLYHWKD |
| 32614 | ISYDYAQAP |
| 32615 | ITMPYAHMC |
| 32616 | KTPWYTWHD |
| 32617 | MASLYPFGQ |
| 32618 | MFTPYRMQA |
| 32619 | NGASYNQIE |
| 32620 | QEMDYRMST |
| 32621 | QHQHYELHQ |
| 32622 | QWSMYNMYD |
| 32623 | RLTCYRMHK |
| 32624 | SAAFYMDVD |
| 32625 | SGLEYMGQQ |
| 32626 | SIQMYRYQA |
| 32627 | TPLWYHRTE |
| 32628 | WCGCYHIFN |
| 32629 | WWFMYWADI |
| 32630 | YPHKYWQMK |
| 32631 | YRHEYAHYS |
| 32632 | TAVLYETEF |
| 32633 | ADHVYGTEQ |
| 32634 | AINEYWDVP |
| 32635 | ASDGYFMEL |
| 32636 | ASGCYATMW |
| 32637 | DIKTYMGLQ |
| 32638 | DKGGYGCHE |
| 32639 | DLVNYGNHG |
| 32640 | DYQWYQVAR |
| 32641 | EMRFYESGN |
| 32642 | EQLQYEPSH |
| 32643 | GILPYVKFQ |
| 32644 | GSTHYTDAN |
| 32645 | ICISYEGGT |
| 32646 | ICQVYSKTG |
| 32647 | IPMQYAHFK |
| 32648 | IPVQYAHFK |
| 32649 | IQDDYQLMT |
| 32650 | LADLYDLSH |
| 32651 | LEQFYEGNI |
| 32652 | LGGDYFRSD |
| 32653 | LKKWYETHG |
| 32654 | MAEPYGLAT |
| 32655 | MCDDYETSQ |
| 32656 | MTKPYMTQC |
| 32657 | MYLGYDMDC |
| 32658 | NQSAYRVAE |
| 32659 | QHEGYVRNG |
| 32660 | QQPSYMVSC |
| 32661 | QTPAYCHHQ |
| 32662 | QYQWYAKIE |
| 32663 | SAHWYCHRT |
| 32664 | THYTYAVAQ |
| 32665 | TNGHYHCKY |
| 32666 | TTTIYRRNG |
| 32667 | TTTIYSRNG |
| 32668 | TYAKYALRG |
| 32669 | VLNTYPCNH |
| 32670 | YIRMYDIAS |
| 32671 | YTMCYSHQS |
| 32672 | YTQNYVFPP |
| 32673 | SIDTYDHRN |
| 32674 | EYSPYQVME |
| 32675 | QHFWYEMES |
| 32676 | QIQLYQNDE |
| 32677 | MCFPYSCAA |
| 32678 | QSEHYVLPC |
| 32679 | RNLNYNTAV |
| 32680 | ATNKYANAI |
| 32681 | LLAKYMVHT |
| 32682 | ITEQYDAFA |
| 32683 | FRLDYDYKN |
| 32684 | LPTQYQQQN |
| 32685 | GQNGYGQCM |
| 32686 | HSDWYMKYP |
| 32687 | NMPAYQIQC |
| 32688 | YISEYVMMV |
| 32689 | NGNHYNAMR |
| 32690 | DPGWYGLAP |
| 32691 | VQMQYERLF |
| 32692 | AYHCYMSWM |
| 32693 | GDVSYDAEA |
| 32694 | MMLGYMEQD |
| 32695 | ESNKWNPYG |
| 32696 | GIHAHNSTY |
| 32697 | KFASANGPL |
| 32698 | LCKTFNQSD |
| 32699 | LGNMENPPP |
| 32700 | LRVLGNMTC |
| 32701 | MFQLPNHFC |
| 32702 | NATQKNGMY |
| 32703 | NPKPNNYQY |
| 32704 | SSDAENRCG |
| 32705 | DAGCSNDNG |
| 32706 | GCLYANIAQ |
| 32707 | GTATTNFDQ |
| 32708 | LLFPRNVGG |
| 32709 | MHRCRNTDQ |
| 32710 | QAMTNNVRC |
| 32711 | TLDVQNVQN |
| 32712 | TSGYCNIGA |
| 32713 | VAAQVNQSQ |
| 32714 | VSNWENVAG |
| 32715 | AASGLNAFK |
| 32716 | AKSCFNGFE |
| 32717 | ASNLMNISF |
| 32718 | ASQWLNDSF |
| 32719 | AWVMNNWWA |
| 32720 | DKSEENRQE |
| 32721 | ETSCVNLFN |
| 32722 | FKESRNQGA |
| 32723 | GAFYINNTF |
| 32724 | GLTLANIGA |
| 32725 | HEPLTNQVS |
| 32726 | HPVDTNAGA |
| 32727 | ITSLCNEFH |
| 32728 | ITSLWNEFH |
| 32729 | MHAERNANM |
| 32730 | MTDQFNKMQ |
| 32731 | MYAERNANM |
| 32732 | MYVWRNDCA |
| 32733 | NFRTINDNM |
| 32734 | NIDIHNHEE |
| 32735 | PLKGTNQIF |
| 32736 | PMVIFNTDY |
| 32737 | QCDYRNMHF |
| 32738 | QGTHHNHVQ |
| 32739 | QKVPVNDHQ |
| 32740 | QMLNHNHHA |
| 32741 | QRMITNWGC |
| 32742 | QTDIRNVSG |
| 32743 | QWQTTNGGA |
| 32744 | RADQSNPCQ |
| 32745 | RHFMINAGR |
| 32746 | RTECCNVAA |
| 32747 | RTSQENKWC |
| 32748 | SAVEGNRHN |
| 32749 | SCFEVNQAS |
| 32750 | SLRNRNADY |
| 32751 | SMVIFNTDY |
| 32752 | SWICCNAST |
| 32753 | SYVHWNWVS |
| 32754 | TAYIHNSEQ |
| 32755 | THNEVNYAQ |
| 32756 | TTDQFNKMQ |
| 32757 | WSHTANKYP |
| 32758 | YILSMNGGE |
| 32759 | YQVKFNVMC |
| 32760 | YRPPCNRKD |
| 32761 | YSTAHNEMP |
| 32762 | DHWQFNTHM |
| 32763 | EAMVINTES |
| 32764 | QHEQSNHIW |
| 32765 | FTSNRNWQA |
| 32766 | RIVDGNACG |
| 32767 | QPQSRNGAR |
| 32768 | KISGLNDAS |
| 32769 | QESMCNYWQ |
| 32770 | TRETMNEWH |
| 32771 | GAMLNNNAS |
| 32772 | RMTPNNCQR |
| 32773 | KIAAWNDFL |
| 32774 | KTGATNLAW |
| 32775 | SQAHQNRTC |
| 32776 | VHHKTNRSY |
| 32777 | STVGQNHCG |
| 32778 | KMTNSNENS |
| 32779 | LPSHCNLGS |
| 32780 | TPQPMNVAT |
| 32781 | KWTPREKDY |
| 32782 | MSQKGFKAG |
| 32783 | RKWMKAKAT |
| 32784 | SGNPTMKNW |
| 32785 | SSVCFPKAG |
| 32786 | TWTMNEKDS |
| 32787 | AHKPAKKWL |
| 32788 | AIWQPSKMD |
| 32789 | ALYYPEKYA |
| 32790 | ETEPLPKQD |
| 32791 | IMTPHWKAC |
| 32792 | ITTEFMKAC |
| 32793 | MIGISMKGC |
| 32794 | MSSALEKCG |
| 32795 | RQIEVTKWG |
| 32796 | TERKMHKAA |
| 32797 | TRASVMKYQ |
| 32798 | AEALHIKAY |
| 32799 | ANLKHAKFG |
| 32800 | DFFYMDKLS |
| 32801 | DIIWAQKAA |
| 32802 | GFNPVIKYS |
| 32803 | GLEWHTKDW |
| 32804 | HQVTIFKDR |
| 32805 | ICWLITKTE |
| 32806 | KMYSSAKSY |
| 32807 | LKHPTWKEE |
| 32808 | LPGYVEKHQ |
| 32809 | MQNACFKDP |
| 32810 | NIYETLKRI |
| 32811 | QKLNIQKQS |
| 32812 | QLMMEEKVS |
| 32813 | RASFNDKAG |
| 32814 | RCVQPQKEG |
| 32815 | SAEHSSKWK |
| 32816 | SLGCFHKWE |
| 32817 | SQWMIWKCR |
| 32818 | TFLCTLKEE |
| 32819 | TFLYTLKEE |
| 32820 | WSAKSTKEQ |
| 32821 | YYSPMVKAH |
| 32822 | ETHQRCKMS |
| 32823 | RTDVMMKVM |
| 32824 | SCSFCPKDC |
| 32825 | ATVHMPKHE |
| 32826 | DWAWTMKIA |
| 32827 | PDLCQDKLN |
| 32828 | LFWQFGKQG |
| 32829 | PRWQSEKCQ |
| 32830 | TFTYEAKFW |
| 32831 | NHDPSIKDQ |
| 32832 | RKWMNAKAT |
| 32833 | IKTTMVKED |
| 32834 | FEEFTTISR |
| 32835 | GGIFEQSSS |
| 32836 | GSFWFTDSK |
| 32837 | IAGPAWFSM |
| 32838 | KQHYAYLSP |
| 32839 | KSTIEPFSI |
| 32840 | MSAECLHSD |
| 32841 | NCIDMWWSS |
| 32842 | PKSEEYTSD |
| 32843 | QHNHMWSSW |
| 32844 | QTMKFTSSW |
| 32845 | RHAPTDISP |
| 32846 | THAVLIRSK |
| 32847 | TQESSRVSS |
| 32848 | TSLKIYHSP |
| 32849 | VEPISSQSY |
| 32850 | WLMKWLESD |
| 32851 | DCIDMWWSS |
| 32852 | DFYTVSMSC |
| 32853 | FMKFIEGSQ |
| 32854 | KTGLPDISA |
| 32855 | LGTFTCSSP |
| 32856 | LSNTCCMSN |
| 32857 | QWQLSWCSC |
| 32858 | QWTQWAMSC |
| 32859 | RCWHIPNSS |
| 32860 | SHGWFHQSQ |
| 32861 | TGAQAANSC |
| 32862 | TWSYNVTSF |
| 32863 | YTVIRCVSN |
| 32864 | ALRVGWTSE |
| 32865 | DKQAMSHSQ |
| 32866 | DTVYNVLSW |
| 32867 | EATDNLTSN |
| 32868 | EDESLHMSW |
| 32869 | EDESRHMSW |
| 32870 | EHVNVTYSP |
| 32871 | ESKSEQPSA |
| 32872 | FLEAWANSM |
| 32873 | FRDGMIVSC |
| 32874 | GIGQPEDSP |
| 32875 | ICATILRSA |
| 32876 | KSGISEMSE |
| 32877 | LGRDCSRSH |
| 32878 | LNMATSLSR |
| 32879 | LSKVQQGSY |
| 32880 | LYQDLLYSE |
| 32881 | NIVESTESP |
| 32882 | NNPVLVASC |
| 32883 | NNTYVMSSG |
| 32884 | QAEYFDASA |
| 32885 | RSMWPPSSG |
| 32886 | TAHCFTVSP |
| 32887 | TASLLWYSN |
| 32888 | TEEKCYISP |
| 32889 | TQLPTMQSL |
| 32890 | TSSPHGASG |
| 32891 | VDRFICASW |
| 32892 | VSSLHDDSE |
| 32893 | DHDFAQFSG |
| 32894 | NNGPIWISS |
| 32895 | PDAMWMASS |
| 32896 | SDRMVSISA |
| 32897 | AAMSSSHSY |
| 32898 | VHSFHVESY |
| 32899 | LRVSQSFSN |
| 32900 | ATMEKCRSI |
| 32901 | LLFPLMSSS |
| 32902 | NTECRQNSD |
| 32903 | YGENISPSP |
| 32904 | TWQSMVNSE |
| 32905 | TSQYSSVST |
| 32906 | GAQSMQFSC |
| 32907 | KTYSAMYSP |
| 32908 | ENTMWQSSQ |
| 32909 | KLKESMESA |
| 32910 | QWMHCQLSR |
| 32911 | MQQHMKYSL |
| 32912 | ECRMAVQSC |
| 32913 | GAANRIQSW |
| 32914 | QRNEMSASR |
| 32915 | DMTLQSVSS |
| 32916 | ENGAISEPA |
| 32917 | HGNLMYTGA |
| 32918 | ISVCKQMIA |
| 32919 | IWQYKDSDA |
| 32920 | LSAFMGRNA |
| 32921 | MTNIAWQKA |
| 32922 | QSAFMGRNA |
| 32923 | QSSLEAGAA |
| 32924 | SEKFIIRTA |
| 32925 | SIGFWMRQA |
| 32926 | TGQWTFATA |
| 32927 | WEFSLADVA |
| 32928 | AIVKMGNPA |
| 32929 | ATNDKVHCA |
| 32930 | DASESHFEA |
| 32931 | DQHEVQNVA |
| 32932 | EWLPCGDLA |
| 32933 | FGRLHQWLA |
| 32934 | GCHSFCEKA |
| 32935 | HCSQMDTYA |
| 32936 | KAESFTYNA |
| 32937 | LITTGEFLA |
| 32938 | MSNSIRWMA |
| 32939 | NAELSCYGA |
| 32940 | NFAYTMLNA |
| 32941 | QNSHVIHEA |
| 32942 | SGTYLYDNA |
| 32943 | TQTKMMDMA |
| 32944 | YMTEWCSWA |
| 32945 | EIVQPQCYA |
| 32946 | AGGQKPAFA |
| 32947 | AISCGKWYA |
| 32948 | ALAWMEFCA |
| 32949 | FFGTPYHMA |
| 32950 | GFELVKAYA |
| 32951 | HTMNGVLYA |
| 32952 | IDSWQPCDA |
| 32953 | KCALIQDIA |
| 32954 | KHAQMSSDA |
| 32955 | KKGDSTREA |
| 32956 | LMMVILDTA |
| 32957 | MAANCEVFA |
| 32958 | MAEYTDAAA |
| 32959 | MWNYGHNYA |
| 32960 | MYGQNWNVA |
| 32961 | NCIHNWQHA |
| 32962 | NHFLASVLA |
| 32963 | NSKIVDYCA |
| 32964 | QCWFRGDYA |
| 32965 | QCWLRGDYA |
| 32966 | RATCGISMA |
| 32967 | RKHGLLFMA |
| 32968 | RMAHTFWDA |
| 32969 | SSFPMWWIA |
| 32970 | SSGLNWRDA |
| 32971 | STGSHGVCA |
| 32972 | WTKWECCVA |
| 32973 | KMLPTCSDA |
| 32974 | KPNPFVAWA |
| 32975 | FGQFCCQVA |
| 32976 | MPEKDGCPA |
| 32977 | DCDWQSAMA |
| 32978 | ALQFVSSTA |
| 32979 | AWDANLAFA |
| 32980 | QKGMMLDCA |
| 32981 | VFGSTRSAA |
| 32982 | RGGWCMRKA |
| 32983 | INENMQWLA |
| 32984 | PPMPVSHFA |
| 32985 | KRTIEHPCA |
| 32986 | RQMCMAHHA |
| 32987 | TSEPHCSYA |
| 32988 | ICNMDFRCA |
| 32989 | AHAWLQHWA |
| 32990 | HIHMSDRGA |

### Example 18

AAV5 Variants with Tissue Tropism in Bone Marrow

This example describes engineered AAV5 variants with tissue tropism in bone marrow that were discovered using the methods and systems described in EXAMPLE 1-EXAMPLE 2.

CNS, liver, skeletal muscle, heart, lung, spleen, lymph node, bone marrow, mammary gland, skin, adrenal gland, thyroid, colon, sciatic nerve, and spinal cord tissues were analyzed to identify amino acid residues in the AAV5 VP1 581-589 region that drive bone marrow tropism. The results are shown in FIG. 18H which shows the positional frequency of each amino acid in the 581 to 589 region of AAV5 VP1 in bone marrow tissue as compared to all other analyzed tissues, wherein the capsid polypeptide has the sequence of SEQ ID NO: 2. The position (“Xaan”, n=1-9) is denoted on the x-axis and amino acid residues are denoted on the y-axis. The frequency of a particular amino acid residue in a particular position is indicated in the heatmap as intensity. Darker squares indicate a higher frequency of a particular amino acid in a particular position and lighter squares indicate a lower frequency of a particular amino acid in a particular position.

The frequency of a given amino acid residue occurring at a specified position corresponding to position 581 to position 589 of SEQ ID NO: 1 (generalized in SEQ ID NO: 2) in variants identified in bone marrow over the frequency of that given amino acid residue occurring at the specified position in variants identified in all other harvested tissues was analyzed to identify a set of sequence rules for capsids that preferentially target bone marrow tissue. With reference to TABLE 35 below, and SEQ ID NO: 2 (AAV5 VP1), the following amino acids can, thus, be independently mutated, in any combination, at any one or more positions Xaa1-Xaa9, to provide a VP1 capsid with enhanced bone marrow tropism as compared wildtype AAV5 VP1 capsid (SEQ ID NO: 1), where bone marrow tropism here refers to properties that are preferred for bone marrow transduction over properties that are preferred for transduction of all other harvested tissues.

TABLE 35
Bone Marrow Tropism Rules

Xaa1 is selected from A, E, G, Q, S, or T
Xaa1 is selected from A, E, or T
Xaa1 is E
Xaa2 is selected from A, I, Q, S, T, V, or Y
Xaa2 is selected from A, S, T
Xaa2 is A
Xaa3 is selected from A, G, I, M, Q, S, or T
Xaa3 is selected from A, Q, or T
Xaa3 is Q
Xaa4 is selected from A, E, P, Q, T, or V
Xaa4 is selected from A, P, or Q
Xaa4 is Q
Xaa5 is selected from F, I, L, M, Q, V, or Y
Xaa5 is selected from F, V, or Y
Xaa5 is V
Xaa6 is selected from F, I, N, Q, S, or V
Xaa6 is selected from I, N, Q, or S
Xaa6 is S
Xaa7 is selected from A, C, M, S, or V
Xaa7 is A, C, or V
Xaa7 is C
Xaa8 is selected from A, C, D, G, M, S, or Y
Xaa8 is selected from A, M, S, or Y
Xaa8 is M
Xaa9 is selected from D, E, G, L, P, S, or Y
Xaa9 is selected from D, E, or P
Xaa9 is P

TABLE 36 below provides sequences of the 581 to 589 region for variant AAV VP1 capsid polypeptides that were found in bone marrow tissue and comport to one or more of the rules provided in TABLE 35. The present disclosure, thus, provides for rAAVs composed of engineered AAV5 VP1 capsid polypeptides having a SEQ ID NO: 2, wherein the Xaa1 to Xaa9 region is any one of SEQ ID NO: 4118-SEQ ID NO: 5117, as disclosed in TABLE 36. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 36
Sequences of the 581 to 589 Region in AAV5 VP1
Capsid Polypeptide that Drive Bone
Marrow Tissue Tropism

| SEQ ID | 581-589 |
|---|---|
| NO | Sequence |
| 4118 | EAEDEIQKA |
| 4119 | EASWPMDWD |
| 4120 | ECIHDQCSS |
| 4121 | EDIDLKGAQ |
| 4122 | EEGPQRMNH |
| 4123 | EGCCYWSHA |
| 4124 | EHWPMAMFN |
| 4125 | EHWQKDYNH |
| 4126 | EIYWMMSNV |
| 4127 | EKKSCQCWH |
| 4128 | ELDSWWRIA |
| 4129 | ELKESMESA |
| 4130 | EMAQTSETP |
| 4131 | ENGHKFNEE |
| 4132 | ENQTRMTSW |
| 4133 | EPTSEVHHD |
| 4134 | EQFNHTWHG |
| 4135 | EQHDWYQKY |
| 4136 | EQIALHCAW |
| 4137 | ERFAFVADH |
| 4138 | ESSRYRMMM |
| 4139 | ETHEFEKLC |
| 4140 | ETRTTAWSA |
| 4141 | EVITWGHMQ |
| 4142 | EVKDKPSIF |
| 4143 | EVWNAHEEG |
| 4144 | EWESSSIIS |
| 4145 | EYTHESHWA |
| 4146 | EIEGMYWPL |
| 4147 | ETAWKWEWS |
| 4148 | EQLLSLCPS |
| 4149 | EKNQSTCET |
| 4150 | EYARGELEN |
| 4151 | EHPEWKCMY |
| 4152 | EMEHYPCYN |
| 4153 | EYSMSEQTN |
| 4154 | EKGGQMCAN |
| 4155 | ECDRLKNER |
| 4156 | EWCPGEPDG |
| 4157 | EEPRILYAG |
| 4158 | EHKLFMKPE |
| 4159 | EKIMVKRAF |
| 4160 | EAKEDWGVR |
| 4161 | EDKYMTKRH |
| 4162 | EGDLSGFQC |
| 4163 | EGKPRCFDW |
| 4164 | EHFQYQNHI |
| 4165 | EHKYFNSGH |
| 4166 | EHWYNRKGL |
| 4167 | EIWMWTQGC |
| 4168 | EKTAGMQTM |
| 4169 | ELPWQRWEN |
| 4170 | ELVPMKLRS |
| 4171 | ENHEEKMAE |
| 4172 | ENLSVNNLF |
| 4173 | ENMDDHYDT |
| 4174 | ENWCIRYAK |
| 4175 | EQHLKPFEA |
| 4176 | EQKHCWSRD |
| 4177 | ERMLVHMRS |
| 4178 | EVQWNNPKP |
| 4179 | HAAGNEFCY |
| 4180 | HACDTQIHP |
| 4181 | HAENGAVWR |
| 4182 | HAISRQQVE |
| 4183 | HALTTDYYN |
| 4184 | HANQIGWCL |
| 4185 | HATSSTGDD |
| 4186 | HAVMHSAVS |
| 4187 | HAYNTFQFG |
| 4188 | IADHTIADP |
| 4189 | IADNIMQAC |
| 4190 | IAGMTADDG |
| 4191 | IAHDGQADS |
| 4192 | IAHIKKVSL |
| 4193 | IAILLHNED |
| 4194 | IANSKMWEA |
| 4195 | IAYCTDAYE |
| 4196 | KACQNSNFW |
| 4197 | KACTDESGF |
| 4198 | KARDLEYIQ |
| 4199 | KAYDTMKCA |
| 4200 | KAYSEILGY |
| 4201 | LAANSVLYF |
| 4202 | LADWYSARI |
| 4203 | LAEIMKCPP |
| 4204 | LAFHLNGTS |
| 4205 | LAGCMGLQE |
| 4206 | LAMAAHEEA |
| 4207 | LAMNQSKAI |
| 4208 | LANLHEWEG |
| 4209 | LASITTPPS |
| 4210 | MAANNECLV |
| 4211 | MAGMMEEGS |
| 4212 | MAGPCSSDT |
| 4213 | MAIIRAEVN |
| 4214 | MAIRWHEVY |
| 4215 | MAIYKCAVN |
| 4216 | MAKYPVNWG |
| 4217 | MANDKHDPQ |
| 4218 | MARQEGWCK |
| 4219 | MASGSVEKM |
| 4220 | MASHIWNQW |
| 4221 | MAVGNLSEA |
| 4222 | NAALHSADW |
| 4223 | NAANQDHTW |
| 4224 | NAEAAWMTF |
| 4225 | NAELMDAHG |
| 4226 | NAFCGNHYL |
| 4227 | NAFNYRLVI |
| 4228 | NAGHYMEYK |
| 4229 | NAGPCWSEE |
| 4230 | NAMPRIQFP |
| 4231 | NAMSNAAML |
| 4232 | NANPHHAVP |
| 4233 | NANQSCANN |
| 4234 | NASHDMHDL |
| 4235 | NAVQTLQLY |
| 4236 | NAWFPMTNT |
| 4237 | PACFRVMFV |
| 4238 | PAMNFITFF |
| 4239 | PAPKNQSTD |
| 4240 | QAACTHPWS |
| 4241 | QADQMPTQE |
| 4242 | QAEGGPNMP |
| 4243 | QAKTSYGGE |
| 4244 | QALVHGAPL |
| 4245 | QAMMAQRAG |
| 4246 | QASQYTVKY |
| 4247 | QATHWPMWR |
| 4248 | RACHPPHLK |
| 4249 | RAEPMIYNF |
| 4250 | RAEQAPVCN |
| 4251 | RAQDTEMML |
| 4252 | RAQLYGMDS |
| 4253 | SAATFKGSP |
| 4254 | SAAYCSYLR |
| 4255 | SAAYFPARG |
| 4256 | SACGAQYPP |
| 4257 | SAEAVVRLW |
| 4258 | SAEVQATTY |
| 4259 | SAGLRVCDR |
| 4260 | SAIFCLKKH |
| 4261 | SAIIVDCHY |
| 4262 | SALDVQRPS |
| 4263 | SALQISMLY |
| 4264 | SANWVQRDM |
| 4265 | SAPSMWWAS |
| 4266 | SAQNHDCWL |
| 4267 | SARPFEFME |
| 4268 | SASNEFDGL |
| 4269 | SAVAHMSET |
| 4270 | TACIAYVDN |
| 4271 | TADFNVRVE |
| 4272 | TAFWMKGPQ |
| 4273 | TAGLSHFKS |
| 4274 | TAGLVTGLG |
| 4275 | TAHCHITYL |
| 4276 | TAHGCPTQH |
| 4277 | TAKTHLFPW |
| 4278 | TALHIDWIP |
| 4279 | TALMSSLQA |
| 4280 | TANLHTIEG |
| 4281 | TANQAYSTI |
| 4282 | TAPTIDYGG |
| 4283 | TATGMKHVY |
| 4284 | TAVHLLDPD |
| 4285 | TAVRYEVCG |
| 4286 | TAWKQNVSD |
| 4287 | TAYYEEFWR |
| 4288 | VAAHEGRKS |
| 4289 | VADRQNEHK |
| 4290 | VAFSLMIMD |
| 4291 | VAGFVSPTG |
| 4292 | VAGNGIPMP |
| 4293 | VAGWATPQL |
| 4294 | VARDHRAGT |
| 4295 | VASLGLYCM |
| 4296 | WAPVHWMKD |
| 4297 | WAYNSVASE |
| 4298 | YAASHMEMP |
| 4299 | YAHIVYRHG |
| 4300 | YAMDCMGGV |
| 4301 | YAQTDGCLE |
| 4302 | YATQNTFSY |
| 4303 | YAVEYGWNQ |
| 4304 | AAHNYEGVA |
| 4305 | CATHMIRTR |
| 4306 | DADDQAVSG |
| 4307 | DADFCPRMP |
| 4308 | DAGLDSAGY |
| 4309 | DAHALQNVI |
| 4310 | DATGKFNFN |
| 4311 | FADKWANQF |
| 4312 | FANLFANTW |
| 4313 | FANLFTNTW |
| 4314 | GANNQNSCE |
| 4315 | GANQHDVGH |
| 4316 | GAQCLFFQN |
| 4317 | HAIHDCYPP |
| 4318 | HAILYFYFP |
| 4319 | LAIQPCDER |
| 4320 | MAGDYACAM |
| 4321 | MASSVVHSC |
| 4322 | MATNREWWG |
| 4323 | MAVWAKAAE |
| 4324 | NARDPACDN |
| 4325 | PADRNNLWQ |
| 4326 | PAECISDSQ |
| 4327 | PATVKPMYW |
| 4328 | QALNRVQFN |
| 4329 | QATGMLYEE |
| 4330 | QATVTKEYW |
| 4331 | RAIHDCYPP |
| 4332 | SARDGISMH |
| 4333 | SAVYIQPDY |
| 4334 | TADVVPCHH |
| 4335 | VADYLNEFT |
| 4336 | VANNGMAVK |
| 4337 | VAWSQEESQ |
| 4338 | YAQQFTMPT |
| 4339 | QAAGETFYG |
| 4340 | AACWTNMQW |
| 4341 | HAPMDNAPF |
| 4342 | NACDWKNDY |
| 4343 | IAMLESWWG |
| 4344 | DAGRFTGTS |
| 4345 | TAPRIDQQE |
| 4346 | VATDSVHES |
| 4347 | MAEQMGCQA |
| 4348 | DARFDQWHH |
| 4349 | PACFDDGEF |
| 4350 | LAEPWATSH |
| 4351 | PAVGFKDEQ |
| 4352 | HANHLMSHE |
| 4353 | LAHCDMMMC |
| 4354 | GADDHHLMT |
| 4355 | YAPKMWAEA |
| 4356 | WANTYSENV |
| 4357 | RAIYNDMTE |
| 4358 | AAMGYAKMS |
| 4359 | AAPIRCYFE |
| 4360 | CAHPEEYHQ |
| 4361 | DAHFPGLAQ |
| 4362 | DAQCFSFGH |
| 4363 | HAVGPVMLE |
| 4364 | GIQDEAWVA |
| 4365 | GQQSFQQVD |
| 4366 | GSQMMQDQA |
| 4367 | GVQLTIQQA |
| 4368 | GVQPALYED |
| 4369 | GYQPDMGMS |
| 4370 | HCQIDVLRV |
| 4371 | HTQFEPGYH |
| 4372 | HVQVDQSKS |
| 4373 | HWQHETMAQ |
| 4374 | INQLKHSAY |
| 4375 | ISQCDTAII |
| 4376 | ISQWCANNK |
| 4377 | KDQCIKQAP |
| 4378 | KIQPNDRTQ |
| 4379 | KVQENEVLG |
| 4380 | LKQQFAEGM |
| 4381 | LVQLNMAGA |
| 4382 | LVQSIWGEE |
| 4383 | LWQLFTMQQ |
| 4384 | LYQQLESSG |
| 4385 | MLQSHTCLG |
| 4386 | MNQFGNMKQ |
| 4387 | MQQPFYRSM |
| 4388 | MYQVWCTDT |
| 4389 | NCQHNEESP |
| 4390 | NDQFRVVDA |
| 4391 | NFQPMHWST |
| 4392 | NIQGVLDYR |
| 4393 | NPQKWNPCI |
| 4394 | NQQCFDYYS |
| 4395 | NVQIATTDS |
| 4396 | NYQPFYVWA |
| 4397 | PHQLHENWG |
| 4398 | PLQLDHECT |
| 4399 | PSQIDHAEQ |
| 4400 | PWQYCELGA |
| 4401 | QCQSEHRDI |
| 4402 | QCQSFGVTQ |
| 4403 | QDQLYGRDE |
| 4404 | QDQQKQYDW |
| 4405 | QHQHLCRTS |
| 4406 | QKQFNAQMG |
| 4407 | QLQWFVKDS |
| 4408 | QNQIEHWCA |
| 4409 | QPQFMVGQI |
| 4410 | QVQFDRCYL |
| 4411 | RFQCEGHLD |
| 4412 | RHQLLEWYD |
| 4413 | RTQPDGQTR |
| 4414 | SDQQWKPTH |
| 4415 | SGQPFSYSK |
| 4416 | SHQQCVMDP |
| 4417 | SIQMMQDHV |
| 4418 | SIQTTFNDD |
| 4419 | STQKYWNYE |
| 4420 | TGQLNTGCV |
| 4421 | THQTARQNV |
| 4422 | TLQWEPKGV |
| 4423 | TNQCICAAP |
| 4424 | TNQNLGNDY |
| 4425 | TSQSMVCCE |
| 4426 | TTQWSQLQS |
| 4427 | TVQSMTSFA |
| 4428 | TVQTPPQYV |
| 4429 | TWQSLCDFE |
| 4430 | VCQLMKYQP |
| 4431 | VDQVFVEDR |
| 4432 | VPQTDLDLC |
| 4433 | VQQIIGTIG |
| 4434 | VSQPCLIVD |
| 4435 | VVQWFAQQV |
| 4436 | VWQQQFVWL |
| 4437 | WDQFCRLMM |
| 4438 | WNQIMEYDY |
| 4439 | WNQPLMWCF |
| 4440 | YCQQTYTEY |
| 4441 | YEQKENQSY |
| 4442 | YGQWFDMSY |
| 4443 | YIQHMHIQR |
| 4444 | YIQTKQIMQ |
| 4445 | YNQAVCQEP |
| 4446 | YPQPFMLMF |
| 4447 | YTQLCHHGV |
| 4448 | AIQSRRSVG |
| 4449 | AKQCLQDYP |
| 4450 | CFQQKHNLF |
| 4451 | CGQSVSHAG |
| 4452 | CQQYTLVQA |
| 4453 | DDQFQDSTC |
| 4454 | FDQPTPDHP |
| 4455 | GEQHQMPMV |
| 4456 | IGQHDVQTT |
| 4457 | IMQQWPAMF |
| 4458 | ISQHTCSKD |
| 4459 | KEQPSQMHG |
| 4460 | MQQANSDYA |
| 4461 | MTQCRDCWL |
| 4462 | MTQDFNCDY |
| 4463 | QRQQMSHDC |
| 4464 | QRQRMSHDC |
| 4465 | RHQLLEWDS |
| 4466 | RVQHCYGDM |
| 4467 | SKQPNADSD |
| 4468 | VCQKYDEWG |
| 4469 | VTQQYWACD |
| 4470 | VTQQYWACN |
| 4471 | WDQPNMHTF |
| 4472 | WGQHKHTVA |
| 4473 | YCQEFEQYP |
| 4474 | YDQWECDIH |
| 4475 | YDQWERDIH |
| 4476 | HDQMMGLER |
| 4477 | TNQCIFKTM |
| 4478 | CHQCIGQDN |
| 4479 | GDQFQDSTC |
| 4480 | THQYFASLA |
| 4481 | IGQHNVQTT |
| 4482 | LKQCQSTMF |
| 4483 | ALQQNPSMT |
| 4484 | MMQCRDCWL |
| 4485 | SCQEWGIQW |
| 4486 | ANQFWKIHC |
| 4487 | GHIQYMRST |
| 4488 | GIIQTGCYK |
| 4489 | GIVQGYEGS |
| 4490 | GKTQFRKCS |
| 4491 | GMYQKCYFA |
| 4492 | GNCQQVQMG |
| 4493 | GQDQCCRIW |
| 4494 | GSSQGRARA |
| 4495 | GSVQEGYDA |
| 4496 | GTRQFEGNC |
| 4497 | GTTQDWRVP |
| 4498 | GVIQDQGMF |
| 4499 | GVVQRATIE |
| 4500 | GVYQKQSMQ |
| 4501 | HDLQYPMSF |
| 4502 | HFHQMIFGG |
| 4503 | ICAQANIAR |
| 4504 | ICDQPGTTG |
| 4505 | IGVQTSFQD |
| 4506 | IHLQWQSMM |
| 4507 | IIDQKYSWC |
| 4508 | INLQGHHWP |
| 4509 | ISCQEEISM |
| 4510 | ISDQNVAHR |
| 4511 | ITLQTANAP |
| 4512 | IVLQYKFME |
| 4513 | IVMQQWFPF |
| 4514 | KCEQIENNP |
| 4515 | KEIQFMTMH |
| 4516 | KLVQNMKRD |
| 4517 | KQDQCMCWY |
| 4518 | KYEQLLGCY |
| 4519 | KYYQAFSKY |
| 4520 | LNWQFVECQ |
| 4521 | LNYQIALCR |
| 4522 | LPHQYNVVY |
| 4523 | LQTQRHWIP |
| 4524 | LSVQGLCSY |
| 4525 | LTSQSNHWG |
| 4526 | LWMQHQHGS |
| 4527 | LWSQYGLWV |
| 4528 | LYLQNMHCE |
| 4529 | MDVQTMNDG |
| 4530 | MSFQETTSI |
| 4531 | MTVQWFDIN |
| 4532 | MWDQTEMFN |
| 4533 | MYSQRNCLY |
| 4534 | NDRQHHHKC |
| 4535 | NNYQKLSFD |
| 4536 | NSIQFSSWR |
| 4537 | NTAQNEGWM |
| 4538 | NTEQMPPLS |
| 4539 | NTVQDMGVC |
| 4540 | NVLQLDAFL |
| 4541 | NWAQAHWYG |
| 4542 | NWDQTMSQD |
| 4543 | PEYQVERSE |
| 4544 | PMIQTDCRW |
| 4545 | PPPQIYLMA |
| 4546 | PWVQTNIGL |
| 4547 | QCFQEGWQD |
| 4548 | QDIQLSHHT |
| 4549 | QEVQFFATD |
| 4550 | QHVQRNYAS |
| 4551 | QQCQTGTPG |
| 4552 | QSCQAIVED |
| 4553 | QTTQQWCMQ |
| 4554 | QYSQIHMQK |
| 4555 | RGYQHEMMG |
| 4556 | RQHQGHYDY |
| 4557 | SNEQLDDDR |
| 4558 | SNIQDDPKG |
| 4559 | SQAQCEMMD |
| 4560 | SSDQQGWVT |
| 4561 | SSLQCFDME |
| 4562 | SSMQMPSGN |
| 4563 | SYVQTEWVY |
| 4564 | TIAQTHWFW |
| 4565 | TIKQCSFCL |
| 4566 | TISQTQGYM |
| 4567 | TQTQINQGR |
| 4568 | TSMQSTQGK |
| 4569 | TTMQRLHFE |
| 4570 | TYAQRHVMG |
| 4571 | VDKQMQIYL |
| 4572 | VHCQADTAN |
| 4573 | VINQNIQCQ |
| 4574 | VMKQGNQEE |
| 4575 | VPAQQSTFV |
| 4576 | VPHQLCHIG |
| 4577 | VQVQGLAIM |
| 4578 | VTAQAESWF |
| 4579 | VTAQQMNLG |
| 4580 | VTVQKQFDH |
| 4581 | VVIQDIRKD |
| 4582 | VVLQQVDPA |
| 4583 | WERQYVPFV |
| 4584 | WIHQQFAEI |
| 4585 | WPCQQQSTW |
| 4586 | WSGQNYNGA |
| 4587 | YFKQFMNCY |
| 4588 | YMNQQNKDE |
| 4589 | YSLQTEVND |
| 4590 | YVSQSSYNY |
| 4591 | YYGQNYMGC |
| 4592 | ARAQIQDEW |
| 4593 | CVMQERQAN |
| 4594 | DLEQAMFMG |
| 4595 | DPCQEWDSE |
| 4596 | FEAQIVLPG |
| 4597 | IHLQWIWAN |
| 4598 | KHDQSGIAN |
| 4599 | LSGQIDEMS |
| 4600 | LYMQAPSEG |
| 4601 | NCLQKMQIW |
| 4602 | NDLQDKSHQ |
| 4603 | NVSQITQDC |
| 4604 | PNNQARGQC |
| 4605 | PVIQPCDER |
| 4606 | RWKQWCWEF |
| 4607 | VSYQTQDML |
| 4608 | YDLQTIKQL |
| 4609 | NITQLQCYC |
| 4610 | FHPQTSDND |
| 4611 | MEWQSEYIS |
| 4612 | DHPQMHFYW |
| 4613 | LVAQQDSCE |
| 4614 | LWKQWCWEF |
| 4615 | LVIQPCDER |
| 4616 | KPVQCACQN |
| 4617 | LIIQHPNMA |
| 4618 | SFLQYHERT |
| 4619 | GLPQSADVI |
| 4620 | DQVQGAHLF |
| 4621 | SGDQHLSEG |
| 4622 | LKSQGCEAY |
| 4623 | SCCQHDNNA |
| 4624 | YHEQCQWHS |
| 4625 | NMMQYQWAI |
| 4626 | MIPQHARGP |
| 4627 | TGRQYYSCC |
| 4628 | GNLYVPSWG |
| 4629 | GVDLVMMLK |
| 4630 | GVMLVGHKY |
| 4631 | HDFHVKRRC |
| 4632 | HNDWVIMAP |
| 4633 | HNVHVTTKW |
| 4634 | ICKLVMKED |
| 4635 | IEIYVNFCT |
| 4636 | IIDYVAHLL |
| 4637 | INELVEWCP |
| 4638 | ISCSVESLS |
| 4639 | KDKEVIVNC |
| 4640 | KDMCVHKYT |
| 4641 | KEWLVDESQ |
| 4642 | KMKMVKEVC |
| 4643 | KVEEVNVQQ |
| 4644 | KWTEVDGSW |
| 4645 | LCEHVQNCM |
| 4646 | LDDKVADHW |
| 4647 | LDKIVGMWS |
| 4648 | LGDNVDCWQ |
| 4649 | LMGDVTHMF |
| 4650 | LRVWVMYLS |
| 4651 | LSCHVQMNS |
| 4652 | LTSDVHIHM |
| 4653 | LYEAVAQME |
| 4654 | MFPLVGMIV |
| 4655 | MMTMVCNAL |
| 4656 | MPASVGSYT |
| 4657 | MWSPVFNWL |
| 4658 | NCEWVTKLA |
| 4659 | NETGVFRQA |
| 4660 | NFCAVEQGV |
| 4661 | NFGYVRAVG |
| 4662 | NIWGVDATE |
| 4663 | NPAPVDQHY |
| 4664 | NQNAVECIQ |
| 4665 | NRDVVRAWH |
| 4666 | NTEHVLVTP |
| 4667 | NVFLVPDMD |
| 4668 | NVVSVPVKS |
| 4669 | PESHVVSVW |
| 4670 | PNYVVQWNF |
| 4671 | PTTIVGWRD |
| 4672 | QCDDVMISE |
| 4673 | QCTFVPFET |
| 4674 | QGKYVGMIM |
| 4675 | QGYYVNSVD |
| 4676 | QMKMVNTSK |
| 4677 | QWCPVQMQC |
| 4678 | QYNVVKCVA |
| 4679 | RCDNVEACF |
| 4680 | RDGPVVPPP |
| 4681 | RIASVQWEE |
| 4682 | RPTLVNAHL |
| 4683 | RQIPVEDSN |
| 4684 | RTDLVAVCG |
| 4685 | RWSHVRVLW |
| 4686 | SFAHVGLPI |
| 4687 | SGHMVVCCR |
| 4688 | SNAPVHTCD |
| 4689 | SPATVAAGC |
| 4690 | SSIGVMGLY |
| 4691 | SSLCVNDQW |
| 4692 | STFRVGDCI |
| 4693 | STTIVGWRD |
| 4694 | TCDYVCAQV |
| 4695 | THTSVGGQC |
| 4696 | TRCDVEGYA |
| 4697 | TTYWVREMS |
| 4698 | TVMTVAAMQ |
| 4699 | TYRLVQQCM |
| 4700 | VFESVVITE |
| 4701 | VFTPVEKSG |
| 4702 | VGDRVQKNS |
| 4703 | VGTTVNVRS |
| 4704 | VHNMVIAFD |
| 4705 | VIGGVTSYW |
| 4706 | VRELVMWGY |
| 4707 | VSGFVFVWR |
| 4708 | VTDYVPMNQ |
| 4709 | VTEIVCESG |
| 4710 | VVGVVIYTR |
| 4711 | VVVAVNADG |
| 4712 | VWELVSHVQ |
| 4713 | YDSGVFTIS |
| 4714 | YYHGVVDSE |
| 4715 | CHAAVTNLY |
| 4716 | DLIIVPWSN |
| 4717 | LEILVAINE |
| 4718 | LMTWVEKGA |
| 4719 | LMTWVEKGD |
| 4720 | NTYGVAFDT |
| 4721 | QYHWVTRII |
| 4722 | RPPHVGPAL |
| 4723 | RQEFVFDCY |
| 4724 | SGKLVMCGY |
| 4725 | SSGPVIILR |
| 4726 | TCFHVAEMF |
| 4727 | PQCHVMADR |
| 4728 | CSDLVGTNC |
| 4729 | IRMNVVLTP |
| 4730 | ISMEVNRGE |
| 4731 | ALDRVSMAC |
| 4732 | FFEGVNINY |
| 4733 | PMYVVPEMC |
| 4734 | GKLDISNLC |
| 4735 | GLCNPSIVT |
| 4736 | GQLYCSRYD |
| 4737 | GSNFGSIDK |
| 4738 | GSNPFSRHG |
| 4739 | GYAKESAKR |
| 4740 | HHMRMSTLP |
| 4741 | HMDDSSICA |
| 4742 | ICREESETP |
| 4743 | IGEKISAMP |
| 4744 | INEGESTYD |
| 4745 | IVNTCSQSW |
| 4746 | KRKEASTRW |
| 4747 | LFEHSSVAN |
| 4748 | LGGLISCPG |
| 4749 | LHISLSKSC |
| 4750 | LISEFSHDK |
| 4751 | LLTHISYME |
| 4752 | LPGIQSTMR |
| 4753 | LVSAYSEKF |
| 4754 | MDIDLSSHA |
| 4755 | MGAFTSINI |
| 4756 | MNVAFSMED |
| 4757 | MTPAMSFSP |
| 4758 | MTTRESICE |
| 4759 | NFDHISAST |
| 4760 | NLRYASNWP |
| 4761 | NMMSKSLDS |
| 4762 | NMSPFSSHS |
| 4763 | NMSPMSESY |
| 4764 | NQYTASNIC |
| 4765 | NTYSWSREW |
| 4766 | QTGAASANR |
| 4767 | QVFCPSVDA |
| 4768 | RGVGMSTDN |
| 4769 | RVAEMSNTP |
| 4770 | SKNCLSFSW |
| 4771 | SMVTDSRWT |
| 4772 | SSGKDSNAA |
| 4773 | STDDESMPG |
| 4774 | TECPKSLTS |
| 4775 | TGKDESHFY |
| 4776 | THFHTSREP |
| 4777 | TMEFTSPCS |
| 4778 | TNHGFSNWV |
| 4779 | TQDWNSTAA |
| 4780 | TTHWQSWIQ |
| 4781 | TYGMCSTFQ |
| 4782 | VCLTHSFSN |
| 4783 | VIEMESGDF |
| 4784 | VNLVDSIRD |
| 4785 | VVNLFSFRS |
| 4786 | WFMEPSPQR |
| 4787 | WMHEDSSQY |
| 4788 | WTFMDSTTD |
| 4789 | YCSPFSGDA |
| 4790 | YLNKRSVNA |
| 4791 | CISMYSEGD |
| 4792 | ISGLMSDYQ |
| 4793 | IWMLPSVTT |
| 4794 | KQHNYSGRA |
| 4795 | KSGLQSITR |
| 4796 | LSEVFSAQR |
| 4797 | LSMVFSKGE |
| 4798 | MHFPWSGEA |
| 4799 | QVPWESKLC |
| 4800 | SKEEDSTML |
| 4801 | VEMCYSWNY |
| 4802 | YYTIASANV |
| 4803 | IPSADSWPM |
| 4804 | LPRTSSQIG |
| 4805 | QWGFGSDET |
| 4806 | AVLLASDQR |
| 4807 | DWNWKSCEW |
| 4808 | TSSNDSTAN |
| 4809 | IHGYHSCYF |
| 4810 | HVMLLSPNQ |
| 4811 | LSELFSAQR |
| 4812 | DIKMFSFHT |
| 4813 | GHTGSWCDR |
| 4814 | GKYRIACDT |
| 4815 | GMADLKCPS |
| 4816 | GMVEAECMD |
| 4817 | GQICILCSE |
| 4818 | GQYFKECYD |
| 4819 | GVNILTCEC |
| 4820 | GWKYEQCTD |
| 4821 | GWTPQLCSS |
| 4822 | HCSSMLCQS |
| 4823 | HNIPFYCMH |
| 4824 | HRLMYDCHK |
| 4825 | HSCPLACMN |
| 4826 | HTIGYQCHD |
| 4827 | HVAEYCCKH |
| 4828 | HYDTHQCWF |
| 4829 | IEWDHLCSH |
| 4830 | IHHGSICCT |
| 4831 | ITETRACAA |
| 4832 | KEEMMQCEE |
| 4833 | KGEPENCST |
| 4834 | KQDMARCWW |
| 4835 | KTDSINCQQ |
| 4836 | LFEPINCYH |
| 4837 | LSEVCLCSE |
| 4838 | MCDANHCNS |
| 4839 | MKGHNDCIS |
| 4840 | MMGVRNCHM |
| 4841 | MSTRCHCRY |
| 4842 | MTYKADCPK |
| 4843 | NDDTMHCAY |
| 4844 | NHTHITCTE |
| 4845 | NMMIMHCHT |
| 4846 | NNTTTRCLM |
| 4847 | NPLMQCCYL |
| 4848 | NRADMICAK |
| 4849 | NSAPSMCST |
| 4850 | NTEPNNCWC |
| 4851 | NTVYDNCGE |
| 4852 | NVKHCHCEI |
| 4853 | NWTYTFCQQ |
| 4854 | PFPMEQCWQ |
| 4855 | QCKWGDCAC |
| 4856 | QVHRPECRH |
| 4857 | RFWLQCCTE |
| 4858 | SFENSICTK |
| 4859 | SFYERACFS |
| 4860 | SGFLTVCDR |
| 4861 | SHCMLNCWA |
| 4862 | SKILFDCTS |
| 4863 | SPLITNCEA |
| 4864 | SQVRTVCGF |
| 4865 | SRNPMMCHP |
| 4866 | STKNDICEG |
| 4867 | STLPSKCGM |
| 4868 | STMGTKCSM |
| 4869 | TDREIHCMN |
| 4870 | TFFPGHCYQ |
| 4871 | TLIEKLCED |
| 4872 | TPMRCYCFG |
| 4873 | TTDTATCKS |
| 4874 | TTHKMTCCP |
| 4875 | TTYKQECTT |
| 4876 | VDEWYVCMW |
| 4877 | VDRHGGCQF |
| 4878 | VFSKFVCQL |
| 4879 | VGSLYNCSG |
| 4880 | VNTLRHCQS |
| 4881 | VPTNLNCCQ |
| 4882 | VREHYKCGW |
| 4883 | YENAMICEM |
| 4884 | YTENKECCP |
| 4885 | AKNYFWCKP |
| 4886 | CISILGCAH |
| 4887 | FTEEGACGM |
| 4888 | GGMDLICHG |
| 4889 | HWVMQMCIG |
| 4890 | IMEPLVCDD |
| 4891 | KGCMDWCDS |
| 4892 | MKYDRPCCT |
| 4893 | NNDHFVCYL |
| 4894 | NTNMRHCGI |
| 4895 | RSIPGGCVA |
| 4896 | SFYSHICRI |
| 4897 | TCHWIQCFN |
| 4898 | TGEAQDCNP |
| 4899 | TVPMDHCWA |
| 4900 | VMKIKRCEG |
| 4901 | VNALFDCCY |
| 4902 | VTMNFHCGC |
| 4903 | VNDYDQCCM |
| 4904 | DYAENWCIE |
| 4905 | AWRPPNCEL |
| 4906 | NPIKFQCAH |
| 4907 | VYANENCAP |
| 4908 | PHEDCECLR |
| 4909 | TSVMWACPG |
| 4910 | GICGTRSMK |
| 4911 | GIMLSQDML |
| 4912 | GIVADRSME |
| 4913 | GLGYGHHMM |
| 4914 | GMVFTNYMR |
| 4915 | GSELGGVMG |
| 4916 | GVLPSYMMM |
| 4917 | HSPPCQIMP |
| 4918 | HWTTEEQMM |
| 4919 | IMCTKARMD |
| 4920 | IMIFEQMMH |
| 4921 | IQGCQMGME |
| 4922 | ISHNNAHMQ |
| 4923 | ITTPYGHMY |
| 4924 | KDLRSMEMC |
| 4925 | KFMGYMVMP |
| 4926 | KFMGYTVMP |
| 4927 | KHWFDPGMW |
| 4928 | KIMEPTFMC |
| 4929 | LCGLNTKMY |
| 4930 | LFHPDMMMP |
| 4931 | LGDDKYIMS |
| 4932 | LNANMKEMF |
| 4933 | LQTSTINMQ |
| 4934 | LSFDFPTMC |
| 4935 | MCALNPGMP |
| 4936 | MCFMQNDMR |
| 4937 | MDWSFGKMG |
| 4938 | MHDGGMEMP |
| 4939 | MILGEMSMY |
| 4940 | MNLNKIMMY |
| 4941 | MPYCEHWMI |
| 4942 | MSIPFCWMQ |
| 4943 | MVKPCGSMF |
| 4944 | NFHRWDRMT |
| 4945 | NIEWFKTMG |
| 4946 | NKEDNHMMM |
| 4947 | NKNEMAMMG |
| 4948 | NNCHYNIMA |
| 4949 | NQFPATEMQ |
| 4950 | NTFLCQKMA |
| 4951 | NTRFNVDME |
| 4952 | PLTLADKMQ |
| 4953 | PWYWPFEMY |
| 4954 | QGNWTMLMQ |
| 4955 | QKECYPNMV |
| 4956 | QSGVDFAMA |
| 4957 | QSLREENMQ |
| 4958 | QVLKGEEMP |
| 4959 | RHDHMMNMA |
| 4960 | RSVVNEKMF |
| 4961 | RVCNNESMF |
| 4962 | SCVVTDNMR |
| 4963 | SDHVLHEMC |
| 4964 | SEREMRHMP |
| 4965 | SFASKIAME |
| 4966 | SLDHMPMMG |
| 4967 | SLTFFQYMD |
| 4968 | SPSSHCVMI |
| 4969 | STSPYEHME |
| 4970 | SVGTRINMA |
| 4971 | THSSLASMV |
| 4972 | TLCSQNWMM |
| 4973 | TNHIQNEMD |
| 4974 | TNNEDTQMH |
| 4975 | TRHYNCTML |
| 4976 | TVVTKGTMP |
| 4977 | TWIEMDYME |
| 4978 | TYPKRMNMM |
| 4979 | VCDPWFAMP |
| 4980 | VDAPQPNMP |
| 4981 | VMYMRDQMW |
| 4982 | VPKWGTGMF |
| 4983 | VSAYEQNMK |
| 4984 | VTWHPLSMH |
| 4985 | VVEPQMRMC |
| 4986 | WITLNQKMV |
| 4987 | WWCHPMHMM |
| 4988 | YDKTYMAMG |
| 4989 | YQFNWGNMT |
| 4990 | YTKETGGMV |
| 4991 | DKIPINGMS |
| 4992 | DVEKTMQMN |
| 4993 | FHFIRHEMV |
| 4994 | FQMAMCSMG |
| 4995 | GRFEEGAMY |
| 4996 | IGVMLNAMQ |
| 4997 | KDRCQYEMA |
| 4998 | MCGFETTMP |
| 4999 | MHDDYRGMQ |
| 5000 | MSRRKEAMP |
| 5001 | QRIWTTYMH |
| 5002 | RRFEEGAMY |
| 5003 | SELFRANMD |
| 5004 | VMCNMMQMT |
| 5005 | WNAPKVMMA |
| 5006 | DKIPINGMP |
| 5007 | SFHPSWWMY |
| 5008 | NCFDSEDMG |
| 5009 | VTTNELGMH |
| 5010 | TTFPQVIMN |
| 5011 | DENWNHNMF |
| 5012 | TSGEMLRML |
| 5013 | LIEVLNHMN |
| 5014 | LFDRCNNMW |
| 5015 | KFHSKERMN |
| 5016 | YMNSLWHMR |
| 5017 | GIVTCAGMC |
| 5018 | GIHICKACP |
| 5019 | GNWSKVSKP |
| 5020 | GWENLFMTP |
| 5021 | HHPTLFHFP |
| 5022 | HPDNSQMSP |
| 5023 | HQTPSELAP |
| 5024 | HSAAPTEGP |
| 5025 | HVFSIFHVP |
| 5026 | IMRLDARCP |
| 5027 | INTGFMNKP |
| 5028 | ISHPSVGEP |
| 5029 | ITMPRMDNP |
| 5030 | KDVEFFHGP |
| 5031 | KSEENYNHP |
| 5032 | KVDDHAFEP |
| 5033 | KVDNEFYVP |
| 5034 | KVWPCNPDP |
| 5035 | LCAIWTLCP |
| 5036 | LFRFDMSPP |
| 5037 | LHCSPEIGP |
| 5038 | LHESLDDFP |
| 5039 | LPCHAIVSP |
| 5040 | LVTLNHNKP |
| 5041 | LYGDAQGGP |
| 5042 | MGGHMHEYP |
| 5043 | MMVCGDRSP |
| 5044 | MPNMWQEWP |
| 5045 | MQEELVHCP |
| 5046 | MRGIEVEQP |
| 5047 | MYIRREGNP |
| 5048 | NCDERPHWP |
| 5049 | NEGPIMYSP |
| 5050 | NEWNPLPKP |
| 5051 | NILCKHKVP |
| 5052 | NISGQLHTP |
| 5053 | NMSGEYVFP |
| 5054 | NQCEIIDFP |
| 5055 | NSTNNGWQP |
| 5056 | NSVKQNAAP |
| 5057 | NTGLSDFPP |
| 5058 | NVTTAWNCP |
| 5059 | NWHWPPVFP |
| 5060 | NYDFYGMIP |
| 5061 | NYGIAYNFP |
| 5062 | PLIIQQFWP |
| 5063 | PSACMAGKP |
| 5064 | QFEDMYKSP |
| 5065 | QHANFAVQP |
| 5066 | QNSPCTAFP |
| 5067 | QSCLFMQIP |
| 5068 | QSTWAERWP |
| 5069 | QTMLYWYLP |
| 5070 | QTWKHMLGP |
| 5071 | RCIWKCEGP |
| 5072 | REDPSALEP |
| 5073 | RHDLYVWSP |
| 5074 | RYDYAKYHP |
| 5075 | SFCTHDHFP |
| 5076 | SGVPYWAHP |
| 5077 | SSIYEPIYP |
| 5078 | STSCSPKWP |
| 5079 | STTSQHHPP |
| 5080 | SVTSQWMPP |
| 5081 | SVVCQQDEP |
| 5082 | TESDFAIGP |
| 5083 | TMTRIVHEP |
| 5084 | TNSSGIFPP |
| 5085 | TSALELQKP |
| 5086 | TWPRGAMHP |
| 5087 | TWSSIDMWP |
| 5088 | VFLVSGMLP |
| 5089 | VGLTMTGVP |
| 5090 | VRNIGGEWP |
| 5091 | WDCTMYHFP |
| 5092 | YPHTPHNCP |
| 5093 | YPHTPYNCP |
| 5094 | AQPNGNEHP |
| 5095 | CISIIAITP |
| 5096 | CISITAITP |
| 5097 | CVLSWCVRP |
| 5098 | DFNDSNWLP |
| 5099 | DMMVSGHVP |
| 5100 | DQLTWHTGP |
| 5101 | DQMDEFLYP |
| 5102 | GIDRPHFVP |
| 5103 | HNNNHAIPP |
| 5104 | LGNWHHNSP |
| 5105 | LHYNNERDP |
| 5106 | MSEVMDFLP |
| 5107 | STAHRPWTP |
| 5108 | TELVRQVAP |
| 5109 | TITALMVDP |
| 5110 | VKLTHRVRP |
| 5111 | VQPVQLFCP |
| 5112 | YTCCENHWP |
| 5113 | YPNDMTFEP |
| 5114 | AQPSMDYVP |
| 5115 | FKVRKFWDP |
| 5116 | QTEEPDDYP |
| 5117 | INEGLLYPP |

### Example 19

AAV5 Variants with Tissue Tropism in CNS as Identified by Machine Learning

This example describes preferred properties of engineered AAV5 variants that display CNS tissue tropism as identified by machine learning methods.

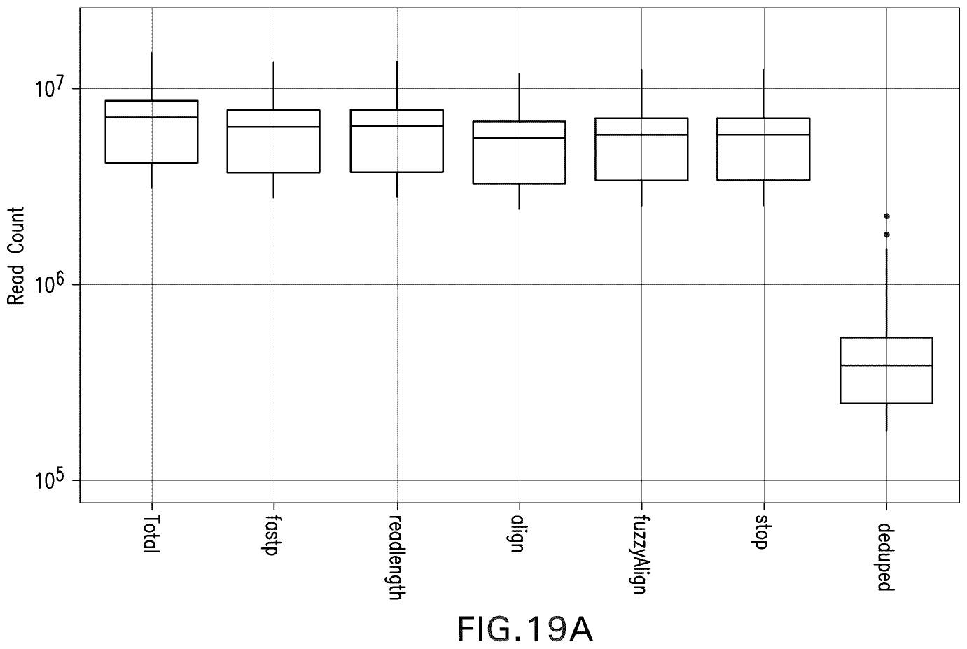

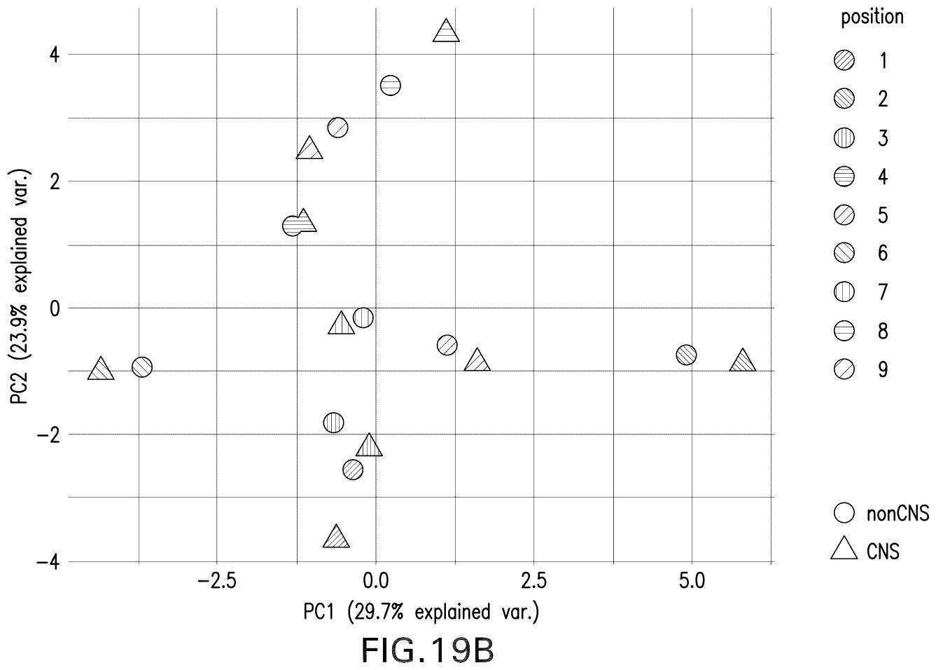

First, sequencing data from CNS tissues was subject to bioinformatics pre-processing. An overview of the bioinformatics pre-processing steps are as follows. First, raw NGS paired end reads were merged, quality-filtered (>Q30 avg), and base-corrected (fastp). Next, reads <150 nt were removed (readlength). Accurate alignment of reads was ensured using fuzzy matching of constant sequences on both 5′ and 3′ of variant region (align, fuzzy Align). Reads with stop codons were removed (stop). Additional relevant filters were used to select variants for subsequent analysis (e.g., “deduped”-shows number of unique UMI-var pairs detected in a given sample). Finally, filtered variants were moved into the machine learning pipeline of the present disclosure. FIG. 19A shows the results at various steps of bioinformatics pre-processing of the NGS data. Stepwise read count for 48 individual samples through filters is shown. FIG. 19B shows a positional comparison of CNS/non-CNS variant sequences. Position 1-9 refers to the 581-589 positions of AAV5 VP1 (SEQ ID NO: 2).

Additional filters were developed taking into consideration the experimental design. First, as the libraries were designed such that a single amino acid residue is encoded by a single codon, each variant amino acid sequence should be represented by roughly one nucleotide sequence. Consequently, the number of sequencing reads for a given nucleotide sequence should approximately match the number of reads of the encoded amino acid sequence (the ratio of sequencing reads for a given nucleotide sequence and its encoded amino acid sequence should be approximately equal to 1). As such, in some cases, DNA variants were eliminated for which this ratio was <0.9. Furthermore, as many errors can arise during library preparation and sequencing, DNA variants were eliminated that were only present once after deduplication of the sequencing reads.

Finally, as variant sequences isolated from tissues must be amplified during library preparation, the abundance of a variant nucleotide sequence in the starting material should be proportional to its abundance after amplification. Thus, variant sequences in the starting material should show greater amplification as compared to variant sequences that arise due to PCR or sequencing errors. Thus, comparing the ratio of sequencing reads of a nucleotide sequence before and after deduplication produced a bimodal distribution, with a higher-ratio group likely representing variants in the starting population, and a lower-ratio group of variants presumably resulting from PCR/sequencing errors. Therefore, as an optional stringent filter, in some embodiments the low-ratio group variants were removed.

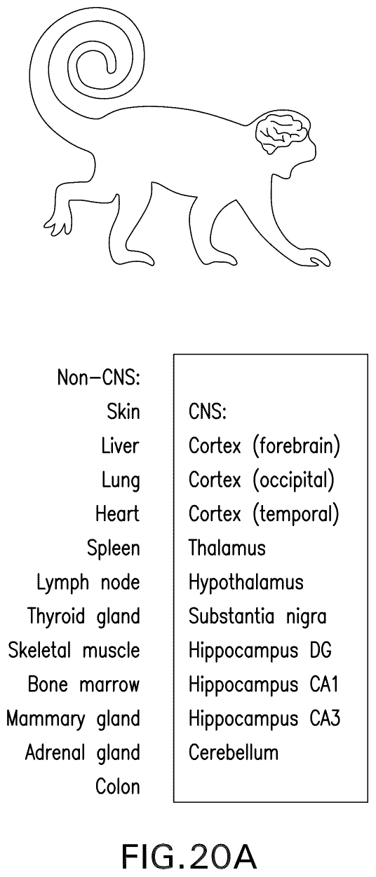

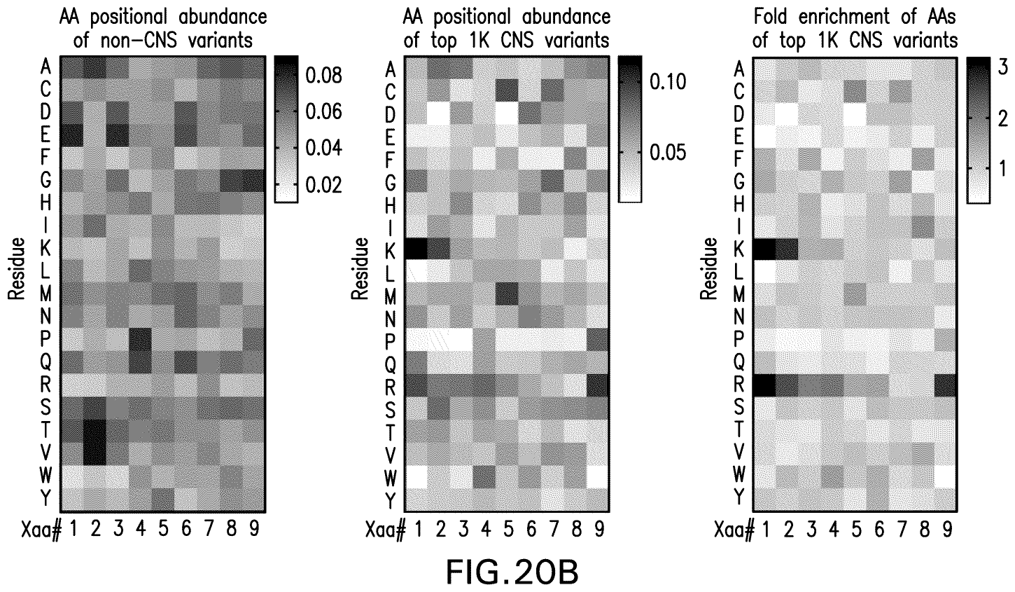

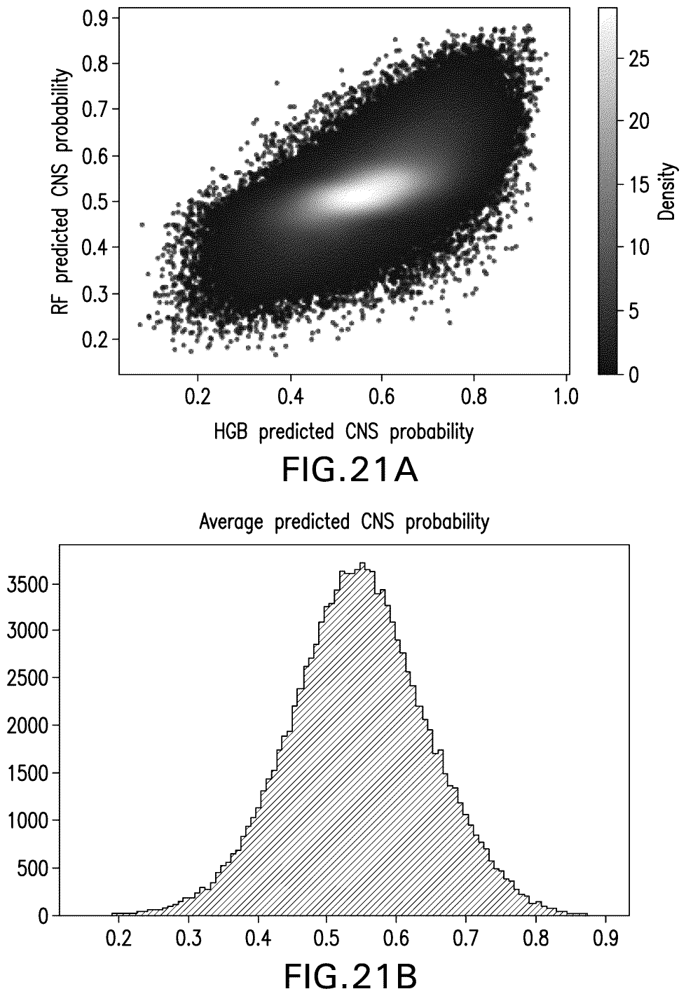

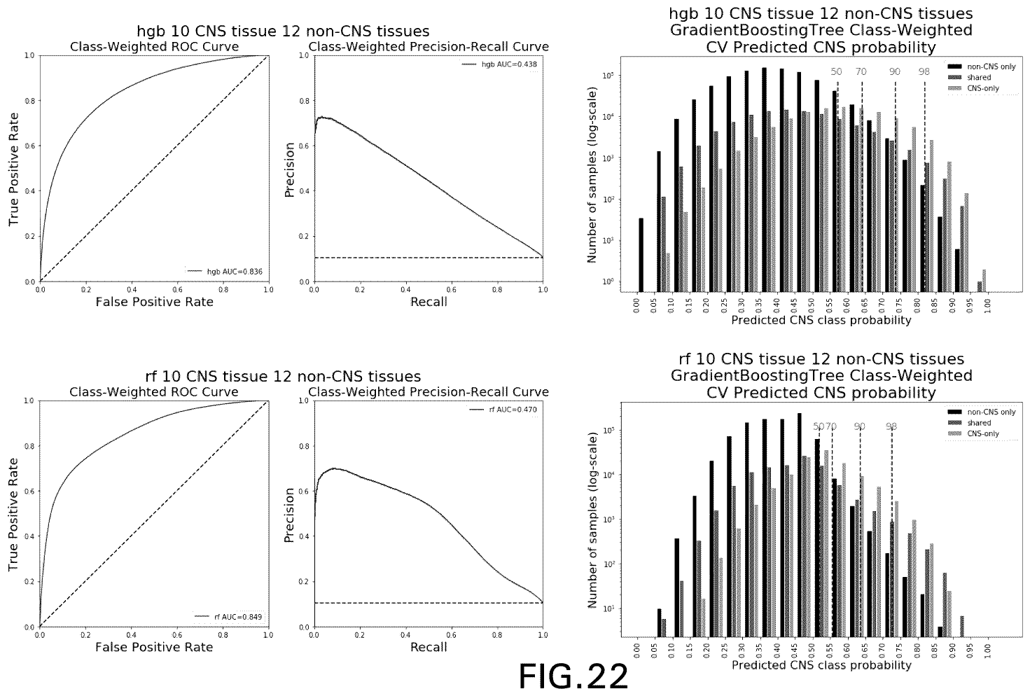

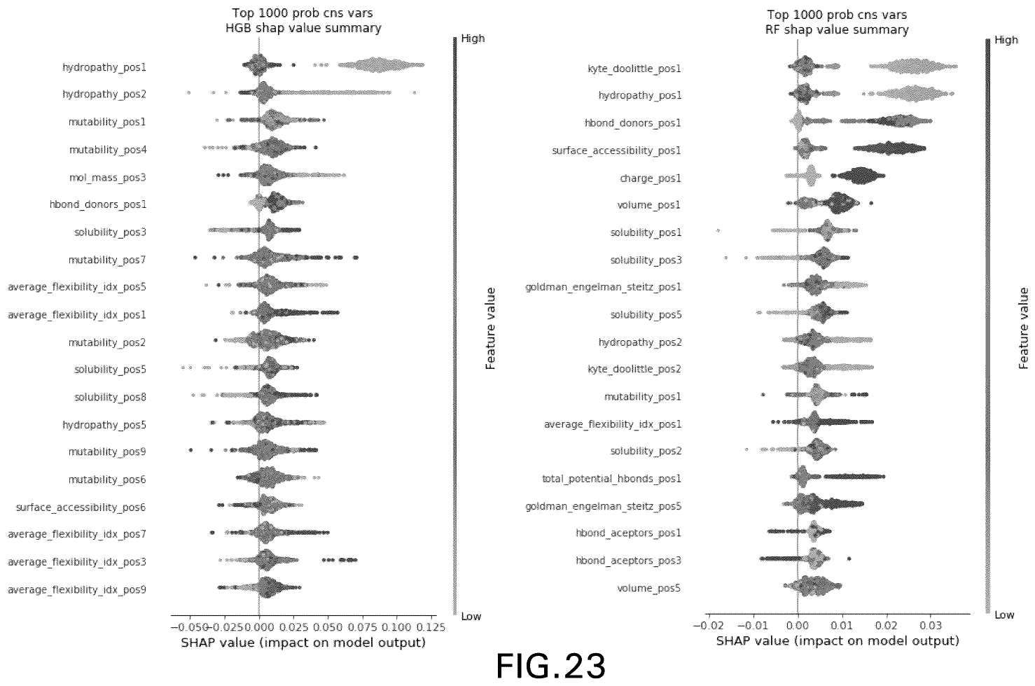

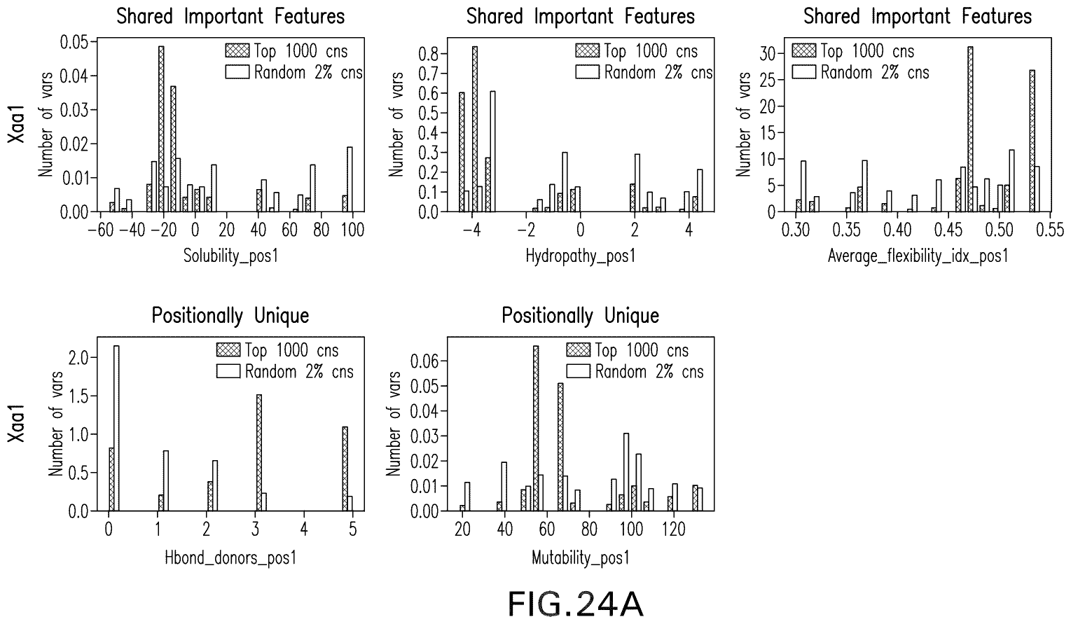

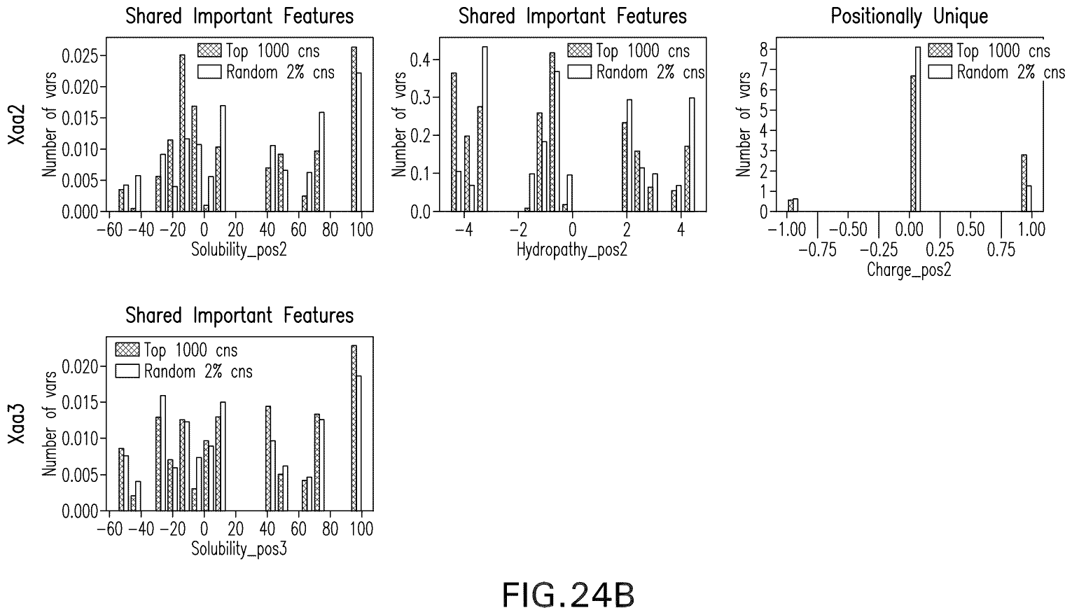

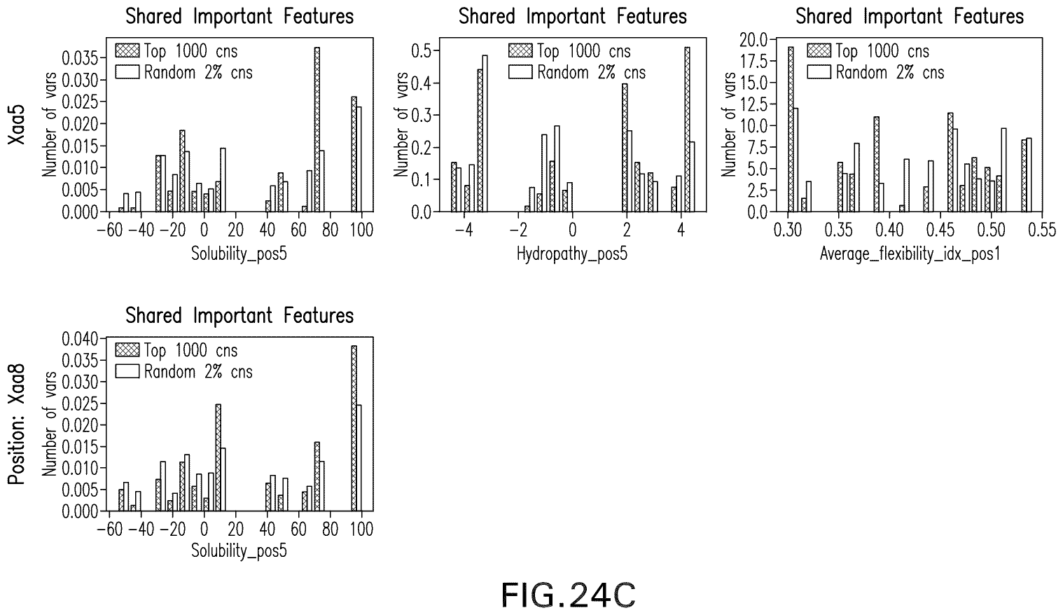

FIG. 20A shows an example of peripheral and CNS tissue samples analyzed by the methods described herein. FIG. 20B shows amino acid positional abundance in the top 1000 predicted/filtered variants recovered from the CNS compared to non-CNS variants. An overview of the machine learning process is as follows. Filtered variants were binary classified as groups of Present (1)/Absent (0) for the targeted tissue(s): those found in both target/non-target tissue(s) (“shared” variants) were assigned as Absent (0). Each 9 aa variant sequence is represented as a 144-feature vector, composed of 16 biophysical properties per position (TABLE 37 below). Featurized variants were input into 2 separate ML models: Random Forest (RF) and Histogram-based Gradient Boosting Tree (HGB). Training and testing was done with cross-validation and all predictions of tissue Presence/Absence were made on hold-out data not used in model training (FIG. 22). Weights were assigned to each training sample to correct for class-imbalance. Machine learning performance was assessed through concordance between two models (FIG. 21A-FIG. 21B) and the average predicted probability of the two models was taken as the prediction. The top predicted variants were outputted. Important features contributing to tissue targeting/de-targeting were extracted (FIG. 23 and FIGS. 24A-C) as Shapley Additive Explanations (SHAP) values, representing the contribution of individual features to model output.

To accommodate potential sparse sampling of recovered variants from tissues, we focus the model on biophysical properties through featurization of the variant sequence. In this embodiment, the biophysical properties listed in TABLE 37 were used to construct a 144-feature vector for each variant as input for the ML. In TABLE 37, amino acid biophysical features are derived from the following amino acid properties. “Charge” refers to the electrostatic property of the amino acid side chain as an acid or base, having a positive or negative charge in an aqueous solvent at neutral pH. “Phosphorylation” refers to whether the functional group of an amino acid residue can have a phosphate group added as a post-translational modification. “Ionic_bond” refers to the capacity of an amino acid residue side chain to participate in electrostatic interactions. “Hydrogen_bond” refers to the capacity of an amino acid residue side chain to participate in hydrogen bond(s), “hbond_donors” refers to the number of amino acid residue side chain atoms that can donate a hydrogen atom to a hydrogen bond under neutral pH conditions, whereas “h-bond_acceptors” refers to the number of amino acid residue side chain atoms that can accept a donor hydrogen atom in a hydrogen bond under neutral pH conditions and “total_potential_h_bonds” refers to the number of amino acid residue side chain atoms that can participate in a hydrogen bond. “Mol_mass”, is the predicted molecular weight of an amino acid residue in unit Daltons. “Volume” refers to the predicted volume of a given amino acid residue in aqueous solution, and is adapted from Zamyatnin, A. A., Protein volume in solution, Prog. Biophys. Mol. Biol., 24:107-123 (1972). “Hydropathy” represents the hydrophobic (repels water) or hydrophilic (attracts water) properties of the side chain of a given amino acid residue, and the values are adapted from Kyte, J. and Doolittle, R. F., J. Mol. Biol., 157:105-132 (1982). “Goldman_engelman_steitz” is a particular measurement of hydrophobicity, as it refers to the free energy transfer from amino acid residues in an alpha-helix from non-aqueous condition to water, and the values for individual amino acids (kcal/residue) are adapted from Engelman, D. M., Steitz, T. A. and Goldman A., Annu. Rev. Biophys. Chem., 15:321-353 (1986). “Flexibility” refers to the symmetric/asymmetric distribution of amino acid residues in polypeptides and is adapted from Bhaskaran, R. & Ponnuswamy, P. R., Int. J. Peptide and Protein Res., 32:4:241-255 (1988). “Mutability” refers to the probability that a given amino acid residue would change in across an evolutionary interval, and is calculated by the relative frequency at which a residue is replaced with another, and in this case Alanine is arbitrarily set at “100” as adapted from Dayhoff, M. O., Schwartz, R. M., & Orcutt, B. C. Atlas of Protein Sequence and Structure, Vol. 5, Suppl. 3 (1978). See also references on the world wide web at: imgt.org/IMGTeducation/Aide-memoire/_UK/aminoacids/charge/and imgt.org/IMGTeducation/Aide-memoire/_UK/aminoacids/abbreviation.html #refs, each of which are incorporated herein by reference in their entirety.

TABLE 37
Biophysical properties used to featurize variant amino acids

| Amino | | | | | | average_{—} | ionic_{—} | hyrdogen_{—} | |
|---|---|---|---|---|---|---|---|---|---|
| Acid | category | charge | hydropathy | solubility | phosphorylation | flexibility_idx | bond | bond | hydrophilicity |
| Y | amphipathic | 0 | −1.3 | 63 | 1 | 0.42 | 0 | 1 | −2.3 |
| W | amphipathic | 0 | −0.9 | 97 | 0 | 0.31 | 0 | 1 | −3.4 |
| D | charged | −1 | −3.5 | −55 | 0 | 0.51 | 1 | 1 | 3 |
| E | charged | −1 | −3.5 | −31 | 0 | 0.5 | 1 | 1 | 3 |
| R | charged | 1 | −4.5 | −14 | 0 | 0.53 | 1 | 1 | 3 |
| K | charged | 1 | −3.9 | −23 | 0 | 0.47 | 1 | 1 | 3 |
| V | hydrophobic | 0 | 4.2 | 76 | 0 | 0.39 | 0 | 0 | −1.5 |
| P | hydrophobic | 0 | −1.6 | −46 | 0 | 0.51 | 0 | 0 | 0 |
| M | hydrophobic | 0 | 1.9 | 74 | 0 | 0.3 | 0 | 0 | −1.3 |
| A | hydrophobic | 0 | 1.8 | 41 | 0 | 0.36 | 0 | 0 | −0.5 |
| G | hydrophobic | 0 | −0.4 | 0 | 0 | 0.54 | 0 | 0 | 0 |
| F | hydrophobic | 0 | 2.8 | 100 | 0 | 0.31 | 0 | 0 | −2.5 |
| I | hydrophobic | 0 | 4.5 | 99 | 0 | 0.46 | 0 | 0 | −1.8 |
| L | hydrophobic | 0 | 3.8 | 97 | 0 | 0.37 | 0 | 0 | −1.8 |
| H | polar | 1 | −3.2 | 8 | 0 | 0.32 | 1 | 1 | −0.5 |
| N | polar | 0 | −3.5 | −28 | 0 | 0.46 | 0 | 1 | 3 |
| Q | polar | 0 | −3.5 | −10 | 0 | 0.49 | 0 | 1 | 0.2 |
| S | polar | 0 | −0.8 | −5 | 1 | 0.51 | 0 | 1 | 0.3 |
| T | polar | 0 | −0.7 | 13 | 1 | 0.44 | 0 | 1 | −0.4 |
| C | polar | 0 | 2.5 | 49 | 0 | 0.35 | 0 | 0 | −1 |

| Amino | Surface_{—} | | hbond_{—} | hbond_{—} | total_{—} | mol_{—} | | goldman_{—} |
|---|---|---|---|---|---|---|---|---|
| Acid | accessibility | mutability | doners | aceptors | potential_hbonds | mass | volume | engelman_steitz |
| Y | 1.089 | 41 | 1 | 1 | 2 | 181 | 193.6 | −0.7 |
| W | 0.808 | 18 | 1 | 0 | 1 | 204 | 227.8 | 1.2 |
| D | 1.283 | 106 | 0 | 4 | 4 | 133 | 111.1 | −9.2 |
| E | 1.445 | 102 | 0 | 4 | 1 | 147 | 138.4 | −8.2 |
| R | 1.475 | 65 | 5 | 0 | 5 | 174 | 173.4 | −12.3 |
| K | 1.545 | 56 | 3 | 0 | 3 | 146 | 168.6 | −8.8 |
| V | 0.606 | 74 | 0 | 0 | 0 | 117 | 140 | 2.6 |
| P | 1.236 | 56 | 0 | 0 | 0 | 115 | 112.7 | −0.2 |
| M | 0.714 | 94 | 0 | 0 | 0 | 149 | 162.9 | 3.4 |
| A | 0.815 | 100 | 0 | 0 | 0 | 89 | 88.6 | 1.6 |
| G | 0.714 | 49 | 0 | 0 | 0 | 75 | 60.1 | 1 |
| F | 0.695 | 41 | 0 | 0 | 0 | 165 | 189.9 | 3.7 |
| I | 0.603 | 96 | 0 | 0 | 0 | 131 | 166.7 | 3.1 |
| L | 0.603 | 40 | 0 | 0 | 0 | 131 | 166.7 | 2.8 |
| H | 1.18 | 66 | 2 | 2 | 4 | 155 | 153.2 | −3 |
| N | 1.296 | 134 | 2 | 2 | 4 | 132 | 114.1 | −4.8 |
| Q | 1.348 | 93 | 2 | 2 | 4 | 146 | 143.8 | −4.1 |
| S | 1.115 | 120 | 1 | 2 | 3 | 105 | 89 | 0.6 |
| T | 1.184 | 97 | 1 | 2 | 3 | 119 | 116.1 | 1.2 |
| C | 0.394 | 20 | 0 | 0 | 0 | 121 | 108.5 | 2 |

Favored biophysical properties and favored amino acid residues at each position in the 581 to 589 region of an AAV5 VP1 capsid polypeptide, which are associated with a higher probability of CNS tissue tropism as predicted by machine learning are described below. Any of the below machine learning-derived positional amino acid preferences described in TABLE 38 can be present alone or in combination with each other or in combination with any of the amino acid preferences observed and described in EXAMPLE 5, TABLE 9.

TABLE 38
Machine Learning-Derived CNS Tissue Tropism Rules

Low amino acid solubility at position Xaa1
Xaa1 is selected from K, R, or Q
Low amino acid hydropathy at position Xaa1
Xaa1 is selected from K or R
High average amino acid flexibility index at position Xaa1
Xaa1 is selected from D, E, R, K, G, I, N, Q, or S
High hydrogen bond donors at position Xaa1
Xaa1 is selected from K or R
Amino acid mutability at position Xaa1
Xaa1 is selected from K, R, P, or H
Low amino acid solubility at position Xaa2
Xaa2 is selected from R, K, Q, or S
Low amino acid hydropathy at position Xaa2
Xaa2 is selected from R, K, D, E, N, Q, H, P, Y, W, S, or T
High amino acid charge at position Xaa2
Xaa2 is selected from R, K, or H
High amino acid solubility at position Xaa3
Xaa3 is selected from A, M, V, W, L, or I
High amino acid solubility at position Xaa5
Xaa5 is selected from C, M, V, W, L, or I
High hydropathy at position Xaa5
Xaa5 is selected from M, V, or I
Low average amino acid flexibility index at position Xaa5
Xaa5 is selected from M, W, F, or C
High amino acid solubility at position Xaa8
Xaa8 is selected from H, V, or I

TABLE 39 below provides sequences of the 581 to 589 region for variant AAV VP1 capsid polypeptides that exhibited CNS tissue tropism and comport to one or more of the rules provided in TABLE 38. The present disclosure, thus, provides for rAAVs composed of engineered AAV5 VP1 capsid polypeptides having a SEQ ID NO: 2, wherein the Xaa1 to Xaa9 region is any one of SEQ ID NO: 8118-SEQ ID NO: 9117, as disclosed in TABLE 39. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 39
Sequences of the 581 to 589 Region in AAV5 VP1
Capsid Polypeptide that Drive CNS
Tissue Tropism

| SEQ ID | 581-589 |
|---|---|
| NO | Sequence |
| 8118 | HWELVRCHD |
| 8119 | SQSRTIIQA |
| 8120 | KMAGIGCWD |
| 8121 | KSIMMSKLD |
| 8122 | ICIRGARTD |
| 8123 | CCKSKEACA |
| 8124 | RVAGEGIQS |
| 8125 | DNYWIIPFA |
| 8126 | TERDHKTIY |
| 8127 | VKHTAVDVL |
| 8128 | RGLSQDCCR |
| 8129 | KMEGNMSEL |
| 8130 | KNPPWICVW |
| 8131 | GVFRPDSFR |
| 8132 | AKCTIQNFQ |
| 8133 | KTVTKEVTP |
| 8134 | RTFYVMGTG |
| 8135 | HWEMIRCHD |
| 8136 | GKVQWSAQA |
| 8137 | VAANSRMMI |
| 8138 | KSPCYMSVQ |
| 8139 | KSGKVWYVL |
| 8140 | PWRARVGAR |
| 8141 | RGLTQDCCR |
| 8142 | RQYFEDSAG |
| 8143 | KSVQVYWHP |
| 8144 | IPINDREWG |
| 8145 | QIRSLITHA |
| 8146 | RMTPHNCQR |
| 8147 | EACYWGHCS |
| 8148 | MMAVIRQKS |
| 8149 | LSAKQRFFC |
| 8150 | KFNTKYQLM |
| 8151 | QNKHNWWIT |
| 8152 | SRLPVHKCP |
| 8153 | IRNRYAQAP |
| 8154 | KVNCRMTIM |
| 8155 | KSYLIANRH |
| 8156 | KTTFDVTCY |
| 8157 | MEDRVFACC |
| 8158 | NLNNLVSCR |
| 8159 | DAIWEHIRL |
| 8160 | KWAIVGYAL |
| 8161 | KFGFMYSEF |
| 8162 | KSTMLEHHA |
| 8163 | GRWMCNEAA |
| 8164 | KDHQLWGVW |
| 8165 | ASSFCKFTS |
| 8166 | PKRIRYRTT |
| 8167 | TKITMPGYQ |
| 8168 | TFHQSDYWA |
| 8169 | GKRVGDTSC |
| 8170 | FAQRDKQDR |
| 8171 | KTYSAMHSP |
| 8172 | TRAYYDSYS |
| 8173 | RMTPNNFQR |
| 8174 | KWGNEGRMS |
| 8175 | TVRHRMDAY |
| 8176 | RQQRNHASS |
| 8177 | TISQIPLVR |
| 8178 | FNWLVRAYI |
| 8179 | MYTGRRCWR |
| 8180 | QCHRGYIYE |
| 8181 | NYFAFWVFI |
| 8182 | AIDCCYSCG |
| 8183 | KIYMQCDRE |
| 8184 | DRKQAGQIY |
| 8185 | RDVGEENSA |
| 8186 | IAKKNFMKT |
| 8187 | RKAYLNYVA |
| 8188 | EMHAFWQRG |
| 8189 | FCKCFVKHC |
| 8190 | RKKVEVVVA |
| 8191 | NTRTGNDRL |
| 8192 | LADCMINHH |
| 8193 | LDTKTLASS |
| 8194 | IVDSKVICV |
| 8195 | RTFMKDMCE |
| 8196 | DAHSPWQVI |
| 8197 | TRHPTITMH |
| 8198 | SSRHRGGCQ |
| 8199 | RCTDHGESI |
| 8200 | SSMCSGSLL |
| 8201 | GKLTFDTER |
| 8202 | RKDQSDLWS |
| 8203 | RSMWSAFKH |
| 8204 | FRLDYNYKN |
| 8205 | RKKVEVVGE |
| 8206 | GYNYYWQHV |
| 8207 | TKLMYNTVD |
| 8208 | KTLHMPESK |
| 8209 | GRPVWCVCM |
| 8210 | MVHRVQGDD |
| 8211 | ATRYQICGE |
| 8212 | DIRWLPYDL |
| 8213 | HMHNLSVKP |
| 8214 | THRTMDDYF |
| 8215 | NYFTFWVFI |
| 8216 | HGCKCQYSF |
| 8217 | VSANEDRAG |
| 8218 | YPVNMMPPL |
| 8219 | GFRRTHVDQ |
| 8220 | CWMLHHRCS |
| 8221 | CVNLMPVNN |
| 8222 | TMRAGLENS |
| 8223 | QGFSTDAKP |
| 8224 | QIVSCDTET |
| 8225 | QKGMMLDYA |
| 8226 | DSHRCLKAA |
| 8227 | NTSEMLMAQ |
| 8228 | GKIARDQLN |
| 8229 | QYWWPTTHD |
| 8230 | KTNFGDAIE |
| 8231 | YIYMMLNLH |
| 8232 | QWFAKGVGE |
| 8233 | QLMSCANVE |
| 8234 | DIEFCMKQY |
| 8235 | DYEKFVKWM |
| 8236 | FCNYAAMSP |
| 8237 | CYWSMTNCT |
| 8238 | KWPVTANLH |
| 8239 | DTRHKWCLE |
| 8240 | FCVKSPRVD |
| 8241 | DAIQWGSWQ |
| 8242 | SALYRRGHV |
| 8243 | FMTVRNWAG |
| 8244 | KISTTWADT |
| 8245 | MHNAHVIIE |
| 8246 | MTGCRETVD |
| 8247 | QYKWHDVGV |
| 8248 | VACDMCLVQ |
| 8249 | KSEWTCCPH |
| 8250 | ARKPCNGWD |
| 8251 | TRYPTIAMH |
| 8252 | TFMFNSKWS |
| 8253 | RAMAYHIYW |
| 8254 | LYNMRMKDR |
| 8255 | DIWWTLTRD |
| 8256 | YHVHMMPPL |
| 8257 | KSGSCAMRV |
| 8258 | TRWTEKRQA |
| 8259 | QLMPCTNVE |
| 8260 | MGAMKSDDF |
| 8261 | AMKSHGCLT |
| 8262 | QNGPILTSR |
| 8263 | YCCNQEPFT |
| 8264 | NWCTMYRYC |
| 8265 | KDPDFYDWP |
| 8266 | LWIKQHVKC |
| 8267 | TRLEIEYMG |
| 8268 | DMTHTGIMS |
| 8269 | SMHMRVCYT |
| 8270 | IANKSNCHK |
| 8271 | VGRFNAYGE |
| 8272 | GLWLNVTPR |
| 8273 | NTHTGNARL |
| 8274 | MVKPCLDEW |
| 8275 | QIKAHSNVF |
| 8276 | LVDRSGRAA |
| 8277 | TSKGKPTCC |
| 8278 | NCYCAYGWP |
| 8279 | IWQSLKKAT |
| 8280 | ARSGINGHA |
| 8281 | NWTRNMQWA |
| 8282 | NCLSRKKFA |
| 8283 | CIMLNGCFP |
| 8284 | KNGATNLAW |
| 8285 | NNCKLAMVH |
| 8286 | KATHCDKYA |
| 8287 | VFNPMLQDT |
| 8288 | ARPEIEYMG |
| 8289 | NSVHVHSSN |
| 8290 | SCPWKCMIV |
| 8291 | CSNDINGRT |
| 8292 | KMGMHDMAT |
| 8293 | KKLVNEGTW |
| 8294 | QLNQWTFAG |
| 8295 | FERRIGWDH |
| 8296 | VWNVMIKMC |
| 8297 | TGGCYRMME |
| 8298 | KPDPRQADQ |
| 8299 | LSCKFGWDG |
| 8300 | KYPFKYYVT |
| 8301 | TMLQKKRQD |
| 8302 | GYAQRSFFM |
| 8303 | ATDGYVAQD |
| 8304 | KMSTYHTYD |
| 8305 | HVADNQVVQ |
| 8306 | AKTFVMSFD |
| 8307 | TCNVSWEHE |
| 8308 | RRTDHCEPI |
| 8309 | RTDYSRLSD |
| 8310 | TDLRKRSNL |
| 8311 | IKAWWHDHQ |
| 8312 | KFKWEACCP |
| 8313 | VNVDCDVQQ |
| 8314 | QYQSTGVVR |
| 8315 | LIHEERKEQ |
| 8316 | ETNVVEDWY |
| 8317 | LACTPRYIF |
| 8318 | HGGCYRMME |
| 8319 | QSVNERHFH |
| 8320 | LWIKQHIKG |
| 8321 | QLMPCANVA |
| 8322 | TQREGETSY |
| 8323 | HNDWQQVFS |
| 8324 | YEDNDVMQI |
| 8325 | ARRAGLENS |
| 8326 | GATWLFRSP |
| 8327 | RTLWALYAI |
| 8328 | DCIICSSKG |
| 8329 | HMALVMQTV |
| 8330 | HSNMLSVHQ |
| 8331 | TNGAQIMHV |
| 8332 | STRPIACQS |
| 8333 | CMYVPADQE |
| 8334 | SPHYFADQW |
| 8335 | GFDLMRQIN |
| 8336 | GHHQCYKAN |
| 8337 | EMNSDYSGI |
| 8338 | SYWHVLLLV |
| 8339 | SADRTVMFH |
| 8340 | RFTVKDGDA |
| 8341 | QCKPGAEGF |
| 8342 | KAIQAFEHD |
| 8343 | ATRYQICDE |
| 8344 | MWWREGAMR |
| 8345 | LFMTYSTRN |
| 8346 | QPPNREAWC |
| 8347 | TPKSAGICC |
| 8348 | KPTYEHRGF |
| 8349 | CNYPIIWDR |
| 8350 | FTGTPPVGM |
| 8351 | QGQTGKYVM |
| 8352 | AHLHNQVIF |
| 8353 | KATVEMQSL |
| 8354 | CVRIGMRGP |
| 8355 | HLSFMMSSF |
| 8356 | TTMWIWFPF |
| 8357 | TTNMKWCAG |
| 8358 | FIQPTWDSY |
| 8359 | RCIWCHMQG |
| 8360 | LVHRVQGGD |
| 8361 | NVRSPAVRD |
| 8362 | LKENWMEWG |
| 8363 | YSASFGCQS |
| 8364 | GLKYWMHGI |
| 8365 | QTGTGHGFE |
| 8366 | QMNYQHEHR |
| 8367 | AHLHNKFIF |
| 8368 | STKQMTKSF |
| 8369 | QEENGKSVT |
| 8370 | RVADEGIQP |
| 8371 | CGLSTHEYL |
| 8372 | RANTESDQP |
| 8373 | QSAEWYLNG |
| 8374 | CWQLFNEQN |
| 8375 | AADGYVTQD |
| 8376 | SANQRHHDW |
| 8377 | KCVEWNAPA |
| 8378 | MHDTMGAWC |
| 8379 | GDNMVQWGS |
| 8380 | DVWCRKLMP |
| 8381 | SYRTLFNGW |
| 8382 | MRQHMKYSL |
| 8383 | PDQYLVVGD |
| 8384 | RELDKENCV |
| 8385 | MTGQCRNGQ |
| 8386 | TSLWQQMAS |
| 8387 | HSMFRHPCP |
| 8388 | GNTKCPSWH |
| 8389 | HECDTVAVN |
| 8390 | ERTGEKGIR |
| 8391 | GSGPVQSEE |
| 8392 | DGWCQKLMP |
| 8393 | QSVNEHHFP |
| 8394 | RAAKYQNSH |
| 8395 | DYERADCWM |
| 8396 | IRNYCRMTA |
| 8397 | LAVWQQKME |
| 8398 | INMSVSKDQ |
| 8399 | ACDKWDCCC |
| 8400 | SQPVCCVCM |
| 8401 | GRALANCAE |
| 8402 | ITTHMNCAD |
| 8403 | MKTTMQDYF |
| 8404 | HCIKVESVG |
| 8405 | HKGTHAFAM |
| 8406 | HYTEMSCDD |
| 8407 | DFWWTLTRD |
| 8408 | SMQYFFKTA |
| 8409 | LNCPSKDGT |
| 8410 | CTHMKNVRT |
| 8411 | ANADNSFCQ |
| 8412 | EERPWEWET |
| 8413 | CSHGTGAMC |
| 8414 | FAIWDYESK |
| 8415 | HLRFIMHEP |
| 8416 | HLMPRSQCF |
| 8417 | QLMVAFLCP |
| 8418 | AHSPFLDHM |
| 8419 | KQAGYDLFH |
| 8420 | DTMQWGSWQ |
| 8421 | SGFFVHTSS |
| 8422 | AKIIAQLCV |
| 8423 | NWTGNMQWE |
| 8424 | QREGNNYLT |
| 8425 | NAFWTQNME |
| 8426 | IKQVFNQIV |
| 8427 | HAVSGPTTG |
| 8428 | NTSGLLMAQ |
| 8429 | MYLSFPVHA |
| 8430 | STLSRHGWG |
| 8431 | QWMWQRLID |
| 8432 | TQGTMDSWY |
| 8433 | RPIHTRQNY |
| 8434 | EWLRQPRMT |
| 8435 | NQQRTNNNG |
| 8436 | QPRTVGVAG |
| 8437 | ASWPCGRNV |
| 8438 | LNSMIMWDN |
| 8439 | ESRSPNNPA |
| 8440 | PTSWLNDLT |
| 8441 | MIRKRFEGQ |
| 8442 | AMWAGLENS |
| 8443 | KLTDIWAHM |
| 8444 | TSYNCGRDP |
| 8445 | HCVKVESVG |
| 8446 | TACGSFHGC |
| 8447 | MSVPKRQGP |
| 8448 | LKQAKQMEP |
| 8449 | DCDVWHANV |
| 8450 | MGDWLGVPT |
| 8451 | FCKHNAQTQ |
| 8452 | TQPKMIGNE |
| 8453 | LSAQFNNQS |
| 8454 | PVLLMRRMF |
| 8455 | ISTICPKIC |
| 8456 | TGSFMGCVD |
| 8457 | MTGCREAVA |
| 8458 | ERGLDNKPR |
| 8459 | NYCINKRAV |
| 8460 | SGQPVDRMF |
| 8461 | QIMSAKVFG |
| 8462 | MFVVNRNWA |
| 8463 | WPDPESEEV |
| 8464 | MNTMLMSNS |
| 8465 | HFVDNQVVQ |
| 8466 | NTRQRYHTW |
| 8467 | TSLVMPSLP |
| 8468 | DYRQCIDWE |
| 8469 | VLRYKCYPS |
| 8470 | HWEIEWVYW |
| 8471 | QVMMGQQTP |
| 8472 | RGCQVKCHT |
| 8473 | EQWKTHQQN |
| 8474 | YSKWARALS |
| 8475 | QCMITISCP |
| 8476 | DVWWQKLMP |
| 8477 | VANTWQVHC |
| 8478 | RMMWQSHCG |
| 8479 | VANITACAN |
| 8480 | PWAVEYSLV |
| 8481 | SYMWPHFPH |
| 8482 | FFEQEAHNR |
| 8483 | LCTGTERQG |
| 8484 | MAVGTVSAK |
| 8485 | QSSSPVMWV |
| 8486 | SSVVMVKQP |
| 8487 | EPRLYCAQA |
| 8488 | YANIHNRHG |
| 8489 | EAYIYLGEA |
| 8490 | PVETARCSG |
| 8491 | NAIPYFIQA |
| 8492 | FCKCFMKHF |
| 8493 | EKLVMYHKC |
| 8494 | YEDCMDMYF |
| 8495 | GIWVWDTYN |
| 8496 | AEWDHKTIY |
| 8497 | TQIQIRQAD |
| 8498 | RCNWQFAQH |
| 8499 | ESRHCKYPY |
| 8500 | SSNCMRRAS |
| 8501 | GVGQFRLLS |
| 8502 | KIDQTQWRR |
| 8503 | HLSFLVSSF |
| 8504 | NAGMSFRNR |
| 8505 | VMNRMEWSV |
| 8506 | LFMGMSDST |
| 8507 | ANVVMRKCQ |
| 8508 | GIYYQWRGQ |
| 8509 | DVHAEHSPQ |
| 8510 | QHPMTFSHE |
| 8511 | EPRLYCTQE |
| 8512 | NAKEQVVNT |
| 8513 | EFCHDDILM |
| 8514 | PLFRSEWLA |
| 8515 | GNMLRNFGS |
| 8516 | HAETDHAGL |
| 8517 | MKMHQLNGC |
| 8518 | SSERFRAEG |
| 8519 | QGGCAPMDS |
| 8520 | REVWITGCG |
| 8521 | SDNGWCAHS |
| 8522 | YCCNQELFA |
| 8523 | TFVYGEWDR |
| 8524 | IFPSKIPCQ |
| 8525 | YVTQMQHLE |
| 8526 | NSRLLIKHP |
| 8527 | KTFPAADMK |
| 8528 | VGHVMIKMC |
| 8529 | GTDHVIVGY |
| 8530 | GAQSMQSSC |
| 8531 | YAVQTNNIM |
| 8532 | WGGMSNNQG |
| 8533 | QMVTQLNWG |
| 8534 | VPKQGAKQE |
| 8535 | DEYQQESFR |
| 8536 | GGTRSGKVV |
| 8537 | CQNTAIRAI |
| 8538 | TIHPPWVTL |
| 8539 | CMVSTPKLA |
| 8540 | RIWWALYAI |
| 8541 | SNTTDVFTW |
| 8542 | WTSSKAVYD |
| 8543 | ESSFQDNVI |
| 8544 | YAHAFHCQP |
| 8545 | RRPPKRGTT |
| 8546 | QMYATMDTY |
| 8547 | EGGQCEKCK |
| 8548 | MGDWLGTPT |
| 8549 | GVKYATLHD |
| 8550 | PWEIEWGYW |
| 8551 | MYLLFPVHE |
| 8552 | HVLNGTGGP |
| 8553 | NIGQSPTQL |
| 8554 | VSCMWCQEN |
| 8555 | TMSVHYGQV |
| 8556 | NAAMITTVC |
| 8557 | HAVNSDCKK |
| 8558 | NIRPCVNQE |
| 8559 | QMECRGGAM |
| 8560 | VGVQCPNGG |
| 8561 | MQGMQTFWT |
| 8562 | QTMGIGGGP |
| 8563 | RSQQDRVMA |
| 8564 | QQGMCVTAI |
| 8565 | AIGPRLIDD |
| 8566 | HGINVELGS |
| 8567 | NVDGRHPMI |
| 8568 | DAARERHWE |
| 8569 | QTGTGHWVE |
| 8570 | TCGQCVNGV |
| 8571 | EGSMFAERQ |
| 8572 | HSCKYLMRV |
| 8573 | MCDWLGAPT |
| 8574 | EVSMFAERP |
| 8575 | ESAITSCAW |
| 8576 | RVCEMRTVD |
| 8577 | AHQGCRPGQ |
| 8578 | ANMISPYGL |
| 8579 | EKHRCAKDR |
| 8580 | TQQRSQATA |
| 8581 | IPMSREFPE |
| 8582 | GNQLITMSG |
| 8583 | YEMTSKCAY |
| 8584 | TIGQIPWVW |
| 8585 | HHGASLGKY |
| 8586 | QMMPQMDCS |
| 8587 | DSWQYYNGL |
| 8588 | VTYYGHYSV |
| 8589 | TTVHMPKHE |
| 8590 | QSHHFLNSA |
| 8591 | TNTMIWNDC |
| 8592 | KAIQASERD |
| 8593 | CRSTGMMRQ |
| 8594 | TSSLTDVIW |
| 8595 | TQAKIECWS |
| 8596 | KYAHSSCED |
| 8597 | NYENNFRAQ |
| 8598 | SPSQKGYGP |
| 8599 | QSHGGVVVQ |
| 8600 | WIQLAILVC |
| 8601 | NTHTGNYRL |
| 8602 | EACYLGHCH |
| 8603 | DEYQQESFP |
| 8604 | YRQQMFERQ |
| 8605 | AQQSTMISL |
| 8606 | AHQTPWAWI |
| 8607 | SISRADLQG |
| 8608 | LNVMSIHRC |
| 8609 | AGRFHKHYA |
| 8610 | GEMTRCFRN |
| 8611 | QVKWTANPD |
| 8612 | KTLSPHVFA |
| 8613 | MGRPGACQA |
| 8614 | FMQPYGHGE |
| 8615 | DCWCYTTKC |
| 8616 | LIRRHCHYP |
| 8617 | QMGVTVVQR |
| 8618 | VCYNQEKAA |
| 8619 | EMNSDRSGI |
| 8620 | HLWMCPPRY |
| 8621 | TASLTDVIW |
| 8622 | LETTMRHGE |
| 8623 | FIGFNNAEA |
| 8624 | QQQRTVFGM |
| 8625 | CYNQFHTYN |
| 8626 | LTISHPKSQ |
| 8627 | AVNRIQQVH |
| 8628 | GWFMIQSMK |
| 8629 | WCNWKFAQH |
| 8630 | MSYLVKGVG |
| 8631 | NTSRELIMP |
| 8632 | TVNPCVNGE |
| 8633 | QMGHQLCVQ |
| 8634 | DERHKWCLE |
| 8635 | MWHELITDQ |
| 8636 | LTRSGPVGQ |
| 8637 | AQFHIHRWQ |
| 8638 | VCTPPLNMQ |
| 8639 | SWSQLQHTA |
| 8640 | VATYIHAMH |
| 8641 | STPQGKQRQ |
| 8642 | TTTITNRLR |
| 8643 | SSLCRTYDN |
| 8644 | MLEKRNHWG |
| 8645 | NSELLRYTF |
| 8646 | CGVGLTQCK |
| 8647 | TGQQIHYKG |
| 8648 | DLEPIMTQA |
| 8649 | PIAQDKVWS |
| 8650 | YSTIQGSGP |
| 8651 | ATTTIQGKA |
| 8652 | ISICAQDIG |
| 8653 | STNPNLTGD |
| 8654 | THELHGHCV |
| 8655 | QEMMIVNCD |
| 8656 | EADTFTLIV |
| 8657 | GMNAYPAHS |
| 8658 | NCRQLGDNM |
| 8659 | ARCSMLDHG |
| 8660 | ASTWMQSYS |
| 8661 | HYCSCHNKY |
| 8662 | VSNVTIGCC |
| 8663 | DNAFMHHHV |
| 8664 | SKVHWDLPC |
| 8665 | NPQHQIDIG |
| 8666 | MWWCESAMR |
| 8667 | FIQCCQDTP |
| 8668 | RMVCDGRID |
| 8669 | ELPRYYGQV |
| 8670 | VTAFTTCHY |
| 8671 | TCSSTEGSG |
| 8672 | EWRTISYGN |
| 8673 | QSTPTLGPK |
| 8674 | GWALIVNDL |
| 8675 | VTILGLNPT |
| 8676 | VTCHWHQGG |
| 8677 | QGSVVHSPS |
| 8678 | NQARENWFH |
| 8679 | HIKVCTLGD |
| 8680 | QYYWFAQFL |
| 8681 | LKVDHKIGD |
| 8682 | DSPAFHQSG |
| 8683 | WSCRSICPP |
| 8684 | QHATTCTHP |
| 8685 | QYQHAVWAP |
| 8686 | LVCSNVDDR |
| 8687 | GKEHMPHHM |
| 8688 | SMSDCEVSF |
| 8689 | DHIWLHWME |
| 8690 | WSTLYLMTS |
| 8691 | MANDQRVDY |
| 8692 | SYWDVLLLV |
| 8693 | KMGTLHNME |
| 8694 | DHHRTSAVR |
| 8695 | MNTPCQQAP |
| 8696 | NSTHNTSYY |
| 8697 | AVSMFAERQ |
| 8698 | STDLSNHFQ |
| 8699 | DSIIKDQSA |
| 8700 | FCGKGANQI |
| 8701 | TQRSTQWSK |
| 8702 | CHYQINKFA |
| 8703 | QATPACPAV |
| 8704 | DVFWPDSFR |
| 8705 | FVIKPNQAT |
| 8706 | CRKSKEVCA |
| 8707 | ASGFGHWWG |
| 8708 | RKRTAGVHF |
| 8709 | TYMYRSDCS |
| 8710 | MSVKKECGG |
| 8711 | WHFLMTFYI |
| 8712 | IADDYFMYH |
| 8713 | RIHDTMSDY |
| 8714 | CNMPQQCME |
| 8715 | TVGMRQAMM |
| 8716 | KKIFCSRDY |
| 8717 | NHDEKMWAP |
| 8718 | LCAVCGGCD |
| 8719 | VHPQTHVTD |
| 8720 | QHEQSNRIW |
| 8721 | EAYATVHMC |
| 8722 | LMHTYVVQQ |
| 8723 | DACAAHDHW |
| 8724 | DIMMKHCRR |
| 8725 | LTQYKALVQ |
| 8726 | MDKHKIGYA |
| 8727 | ESDWLLGTI |
| 8728 | NVGYYKWQS |
| 8729 | IECFPRESI |
| 8730 | LSMPYAPTT |
| 8731 | AVKILMHDH |
| 8732 | THELHGHCS |
| 8733 | NSQTIMLHQ |
| 8734 | HIHMSDRGT |
| 8735 | TGRAWMWHS |
| 8736 | NSHAHGHMK |
| 8737 | DYTIMPYIA |
| 8738 | SVTYHERHP |
| 8739 | CTAMGWVHN |
| 8740 | SVTYHKWHP |
| 8741 | VSCEFGQEY |
| 8742 | KDLEYQRSN |
| 8743 | AKGPHWGQG |
| 8744 | RYHHSCDSH |
| 8745 | ACTWLHGTN |
| 8746 | SIHNVLVQN |
| 8747 | KVDQNTNWC |
| 8748 | FGEFTTDEN |
| 8749 | VWMRFDYVD |
| 8750 | MAKKREVGV |
| 8751 | MDEHFSVGC |
| 8752 | EFRCRLIDS |
| 8753 | NHCHCIAHP |
| 8754 | IIMLGCTGR |
| 8755 | VTLTSKMCQ |
| 8756 | RAECRYQTT |
| 8757 | FDVHKMCIY |
| 8758 | VSADTPYMN |
| 8759 | THRTRHTDC |
| 8760 | DYYLNRASP |
| 8761 | SVSTKPHAE |
| 8762 | VADWIGRTP |
| 8763 | DLYIVMNQA |
| 8764 | NPIPGYFEQ |
| 8765 | SCKWIQSNR |
| 8766 | QWGVISGHA |
| 8767 | ESEFGHWWG |
| 8768 | KKAGAKDAL |
| 8769 | CTCEPRSAW |
| 8770 | YLYHSMMKQ |
| 8771 | ILRTIPNRT |
| 8772 | IEESIQNAD |
| 8773 | AMMPKTETH |
| 8774 | VVTLHSKAR |
| 8775 | VCCSHADSA |
| 8776 | DGSSHFTMQ |
| 8777 | VAYPIQTDA |
| 8778 | YAQQSPHCE |
| 8779 | YMLHAIMES |
| 8780 | AAVITINGL |
| 8781 | LNMVWGQKF |
| 8782 | NALPAQQHH |
| 8783 | QKPTIWDHF |
| 8784 | YQDIRQCIF |
| 8785 | TCTHFEFVD |
| 8786 | TLEPAYWHE |
| 8787 | PVHFWTQSK |
| 8788 | RCKRAVQVV |
| 8789 | WCESWGPQG |
| 8790 | AVVYKMNYH |
| 8791 | KNHMMLYVN |
| 8792 | ANMCGCDSF |
| 8793 | LFSDHGVSY |
| 8794 | TWSCCLHQY |
| 8795 | WTDSDSDCT |
| 8796 | VIDCCYSCD |
| 8797 | PIAQDQVLS |
| 8798 | LCRWYRYAN |
| 8799 | QNIRPGVPT |
| 8800 | RTTDHRADE |
| 8801 | QIHSEHCTT |
| 8802 | QSHDGVMVQ |
| 8803 | DLLANTICT |
| 8804 | SANIVFSVC |
| 8805 | LSMTRELWH |
| 8806 | MLHKYLHMW |
| 8807 | QYAITYQKG |
| 8808 | VQMYWSSKD |
| 8809 | GFNLMHMAG |
| 8810 | LFYSPMHTH |
| 8811 | VKAKKMANP |
| 8812 | NCMPTIAGS |
| 8813 | SHWQMQVMA |
| 8814 | HIMQEGRSS |
| 8815 | HFAMKCHNN |
| 8816 | EIIQAHLVN |
| 8817 | VKNALCQHM |
| 8818 | KTWDAHRSS |
| 8819 | IRGFMQNIY |
| 8820 | VSYAPMDAQ |
| 8821 | SGSMTQHHT |
| 8822 | AICTTASFD |
| 8823 | YPTECNHLT |
| 8824 | QSQHLAYKG |
| 8825 | LKAQAVTVT |
| 8826 | CTVQFELGC |
| 8827 | GTSQLQHMA |
| 8828 | PIYCNALCS |
| 8829 | VSKPDMHVS |
| 8830 | GPLVCISAG |
| 8831 | HKVKFGALH |
| 8832 | TQGWLAAGH |
| 8833 | GPRLIVHKL |
| 8834 | MFGFSYSDG |
| 8835 | TMGMANCGM |
| 8836 | QSAIMSART |
| 8837 | VWGMAYTAC |
| 8838 | DATFMKNAF |
| 8839 | TEGPMHRVW |
| 8840 | FFRICDSYI |
| 8841 | CTALVNEDL |
| 8842 | SQAHQNRAC |
| 8843 | AMIGHGAYA |
| 8844 | HAETHHVGL |
| 8845 | ATVDVKGSH |
| 8846 | SSDGESHYI |
| 8847 | RTLLPNANS |
| 8848 | ITCNWCVMP |
| 8849 | GSLLQGVSD |
| 8850 | FCIMASDMA |
| 8851 | QGAFQMMLP |
| 8852 | FMWTTHRWH |
| 8853 | MFAQQMGVD |
| 8854 | ALVMIILQG |
| 8855 | ATISTESEV |
| 8856 | LTCNWTHHG |
| 8857 | TFKEYQGYH |
| 8858 | IRQPIHMTP |
| 8859 | QTAMWCWQE |
| 8860 | HIEVCTLGA |
| 8861 | QTHLKLECE |
| 8862 | GRNEQGYCH |
| 8863 | TFMQNTYQG |
| 8864 | LTNYHIAQE |
| 8865 | ATTEKCRSI |
| 8866 | ENAQMKCLM |
| 8867 | HKLWYWAFF |
| 8868 | AHAFRHKHF |
| 8869 | KFICTTITC |
| 8870 | QPGIVICKK |
| 8871 | IMGYKCKWL |
| 8872 | QPGYMNKLW |
| 8873 | QCDAALNWS |
| 8874 | ISTVCPKIF |
| 8875 | NDQHAPCTE |
| 8876 | ASSHRHWVY |
| 8877 | AMMLFQTSY |
| 8878 | SKEDLCHQQ |
| 8879 | AKIHGAWNA |
| 8880 | ASAPRVNEQ |
| 8881 | SCPWKCPIV |
| 8882 | PNRYRPFCC |
| 8883 | DHLRDHPLY |
| 8884 | GNGTVGKAN |
| 8885 | EQAGAASMW |
| 8886 | VQKLTLNAK |
| 8887 | GHNHMYTQP |
| 8888 | VSKTPWERD |
| 8889 | QSCHVMNMP |
| 8890 | IVLWFVNTT |
| 8891 | WCQHIPHNG |
| 8892 | CIAREYTGY |
| 8893 | YFVVNHFWS |
| 8894 | STLEDEKPY |
| 8895 | ARCHDMWGH |
| 8896 | MAAIKWYMS |
| 8897 | WMLHGWATL |
| 8898 | MVDCMKWHE |
| 8899 | AGMYSFSGE |
| 8900 | VALQTKIEQ |
| 8901 | GTQNLLHGY |
| 8902 | MTQYKALAQ |
| 8903 | GCATYSCYV |
| 8904 | QGDMGTWVG |
| 8905 | SAVVSMRND |
| 8906 | YPYGKCSNY |
| 8907 | RVINTEWGR |
| 8908 | TTYHHGNTL |
| 8909 | QQTTYDCLD |
| 8910 | LTHLQHSAT |
| 8911 | YMPLAMQYC |
| 8912 | TREPCQQCD |
| 8913 | IQDLQIAIK |
| 8914 | ATTNHDMQQ |
| 8915 | FCNKLTSVW |
| 8916 | EAVCMDTFP |
| 8917 | SAALVADGP |
| 8918 | QPEIFPVMA |
| 8919 | HTESGQDET |
| 8920 | TRNTWQQDF |
| 8921 | GVNTNMNGS |
| 8922 | CQAELIDVR |
| 8923 | TSTMCSWSM |
| 8924 | NAEPQQADI |
| 8925 | IRQMAGGHC |
| 8926 | LNQCAQAGC |
| 8927 | TATCGIEAE |
| 8928 | TNLHNNHYW |
| 8929 | AYSYYSVLP |
| 8930 | STMLLLARD |
| 8931 | VAYYSGHMM |
| 8932 | IQQLSMVGS |
| 8933 | ARVKETCEW |
| 8934 | TFMFNSEGS |
| 8935 | CVPKTIACC |
| 8936 | ACYEFVNAC |
| 8937 | TRMQFNKCH |
| 8938 | SRDIAKSST |
| 8939 | YESCNLTRD |
| 8940 | STQPACAWP |
| 8941 | MVGRNMWAV |
| 8942 | CQMQVLNNI |
| 8943 | YLVHEGHSR |
| 8944 | TGTQFMRWA |
| 8945 | MCMQEDRLA |
| 8946 | LATNHRHWL |
| 8947 | GRWPYFLAC |
| 8948 | IPSFYWGDY |
| 8949 | GDDWIIWQA |
| 8950 | MECIHMRND |
| 8951 | SIWDCNHRS |
| 8952 | NTVFQLYHT |
| 8953 | GYGHTFWDA |
| 8954 | VHEEYSNSH |
| 8955 | GTLACDQKE |
| 8956 | FCCMYCQHP |
| 8957 | ESSMWCTDC |
| 8958 | QTADWCHPV |
| 8959 | MPHGRCNTI |
| 8960 | GITQHFVKH |
| 8961 | AMDCGQSFG |
| 8962 | LAYSQYVGC |
| 8963 | NRNCTDAWH |
| 8964 | RSHFQEGVQ |
| 8965 | MFMAKQNAM |
| 8966 | PIWSVYQDE |
| 8967 | DKRIICQDN |
| 8968 | MFYAMVGPE |
| 8969 | ETCCSEWHV |
| 8970 | MCKNGDSHG |
| 8971 | AIHAHTAAN |
| 8972 | NVSSHQHCW |
| 8973 | CRCCWYQDQ |
| 8974 | DMSDCEVSF |
| 8975 | SMCWFNQVA |
| 8976 | AAPMVGEFY |
| 8977 | SPMSFLNVL |
| 8978 | TKGPKELMA |
| 8979 | EGWTQFRPE |
| 8980 | QAGDSMWAK |
| 8981 | SADTNVVVE |
| 8982 | LKSGQVRFT |
| 8983 | YIHDYSSPN |
| 8984 | QTEIAMGFT |
| 8985 | QLCIPLTWE |
| 8986 | EANCNHVGV |
| 8987 | SGMKGRTQI |
| 8988 | AEAIINDIW |
| 8989 | LTEFTPYIG |
| 8990 | FCAHNFTSD |
| 8991 | SFLMMMNCT |
| 8992 | LCMPADRQS |
| 8993 | MTNSQHQHW |
| 8994 | PKHHVTWED |
| 8995 | VSQPAWQQK |
| 8996 | QAQSHIHYT |
| 8997 | EVLMAMSLF |
| 8998 | GQALDKQYQ |
| 8999 | SNEPLDTCY |
| 9000 | SQAVRKCCD |
| 9001 | QGSPLDSFQ |
| 9002 | LLFPLMGSS |
| 9003 | PNSPGVCSC |
| 9004 | MSTHVLKWV |
| 9005 | FCHAFPDAY |
| 9006 | VIGWHGTNA |
| 9007 | TQGVSTIGV |
| 9008 | NDEQRNTLY |
| 9009 | TSQHKYAEE |
| 9010 | HDCIWMECR |
| 9011 | DFAYRSTGV |
| 9012 | DSATSKSAD |
| 9013 | IPASCGSTR |
| 9014 | SMGWDHMMS |
| 9015 | MASTESDMC |
| 9016 | QMEVHWHMR |
| 9017 | AQWISPWPT |
| 9018 | QENDANRPQ |
| 9019 | LVNQLFHSW |
| 9020 | ACDQVNTFQ |
| 9021 | MKSVFMHEW |
| 9022 | WGIKCLGTS |
| 9023 | VSRSDWTMP |
| 9024 | TSTIPGVGT |
| 9025 | YHTFVECAQ |
| 9026 | ASCYVWGPA |
| 9027 | QHQSVMGFA |
| 9028 | HMQAQIHYW |
| 9029 | KQHELIVRV |
| 9030 | AYCCFNRFQ |
| 9031 | TYMQCMVSG |
| 9032 | CAVNTYQKH |
| 9033 | RNINEYEHY |
| 9034 | SARVNHGRV |
| 9035 | HSHMYDCQQ |
| 9036 | NTECWQNSD |
| 9037 | LNCQKANQN |
| 9038 | TVMMLMDNG |
| 9039 | MTTGLMAYE |
| 9040 | LLYRNMILS |
| 9041 | QSHLSDKMY |
| 9042 | TMAMLGWMN |
| 9043 | GLWHGFYAV |
| 9044 | GGCCGGELN |
| 9045 | LQYSPGQQT |
| 9046 | TNNCYWCAP |
| 9047 | AVLGSNYLK |
| 9048 | MDTKVVYGT |
| 9049 | WADRQGALA |
| 9050 | VITLKNNCH |
| 9051 | AIAEIKIQS |
| 9052 | VTMPMATWH |
| 9053 | VHFIAWRGM |
| 9054 | VIGVEMKCM |
| 9055 | TDAGMFAHA |
| 9056 | ITKFKMQDH |
| 9057 | AMCIVCIAP |
| 9058 | KSGYQCDSM |
| 9059 | LALMIQQAH |
| 9060 | LSAQDTMFH |
| 9061 | YQAQLNNCW |
| 9062 | SCEATMHAI |
| 9063 | TILGIPSSK |
| 9064 | DMTHTDIMP |
| 9065 | LIGPNTKWG |
| 9066 | EARMPWAHH |
| 9067 | HVHMCTPSV |
| 9068 | LNTYYRLED |
| 9069 | INCQTKKEM |
| 9070 | VPEPKGTLN |
| 9071 | STHMAGHYA |
| 9072 | APGMAFGGQ |
| 9073 | SVSAQQTMQ |
| 9074 | RTVYDVQDD |
| 9075 | GCYGNQPPT |
| 9076 | TATWGIEVE |
| 9077 | ECATNVGDR |
| 9078 | WDQKNLMDT |
| 9079 | ERTLTKTGS |
| 9080 | IVNMNHSAM |
| 9081 | FWMKWESYG |
| 9082 | AMRDNSVAH |
| 9083 | CWSYTNKNC |
| 9084 | QANPYNHGI |
| 9085 | IHVKFPPFD |
| 9086 | EYIQHSSAR |
| 9087 | TTTTPCNMP |
| 9088 | CCTGLGSMG |
| 9089 | TVTHMSTWG |
| 9090 | NNMLHGAYR |
| 9091 | WGQHLPMID |
| 9092 | MMKMCEYDG |
| 9093 | GANNMTAMN |
| 9094 | THAAYGTNL |
| 9095 | LSSLPMHYC |
| 9096 | ITEQYDAFV |
| 9097 | YTGIKVMGR |
| 9098 | DVEHVSCPK |
| 9099 | WHCEFTQHH |
| 9100 | LSTQSAKEY |
| 9101 | ISDPNVGEP |
| 9102 | SVCSKWDGT |
| 9103 | NNMWDTIFM |
| 9104 | NHKMVRDAL |
| 9105 | HVGIYEHCH |
| 9106 | SPKMMVSMT |
| 9107 | WMVRQKYMA |
| 9108 | LQWWIRMGK |
| 9109 | CCGIRIEYG |
| 9110 | FAGFTPGWM |
| 9111 | PHKDSMQMT |
| 9112 | DKTVQSLSL |
| 9113 | PWACEHVWH |
| 9114 | EYCEFMSEA |
| 9115 | YGVMCPSHY |
| 9116 | PIIRGRSDS |
| 9117 | EPMEANQCR |

### Example 20

Engineered AAV5 Variants with Tissue Tropism in Liver as Identified by Machine Learning

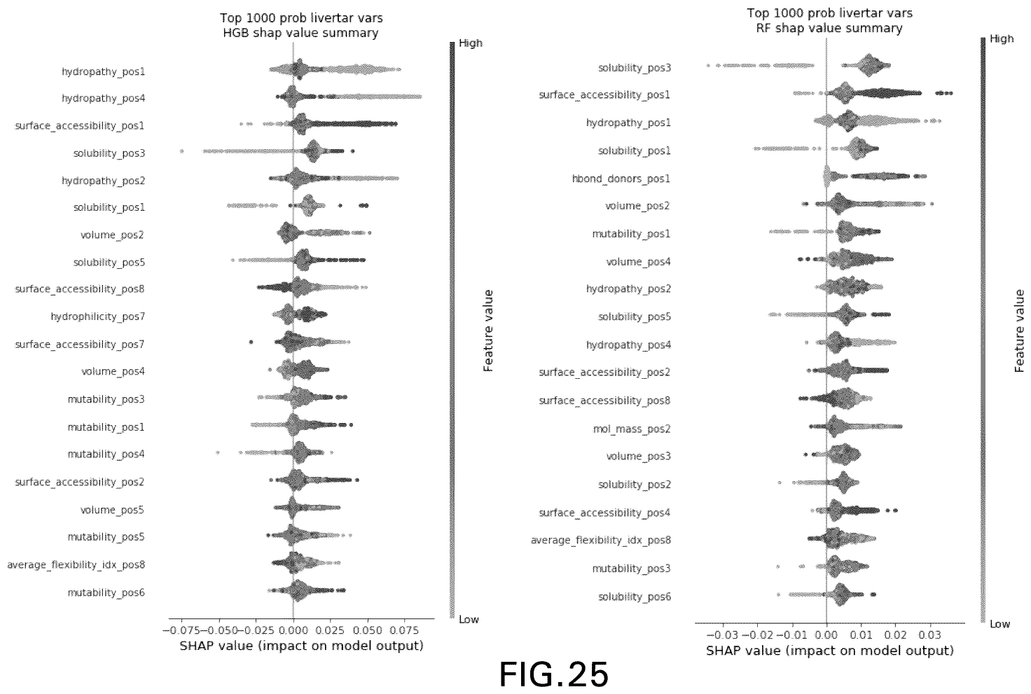

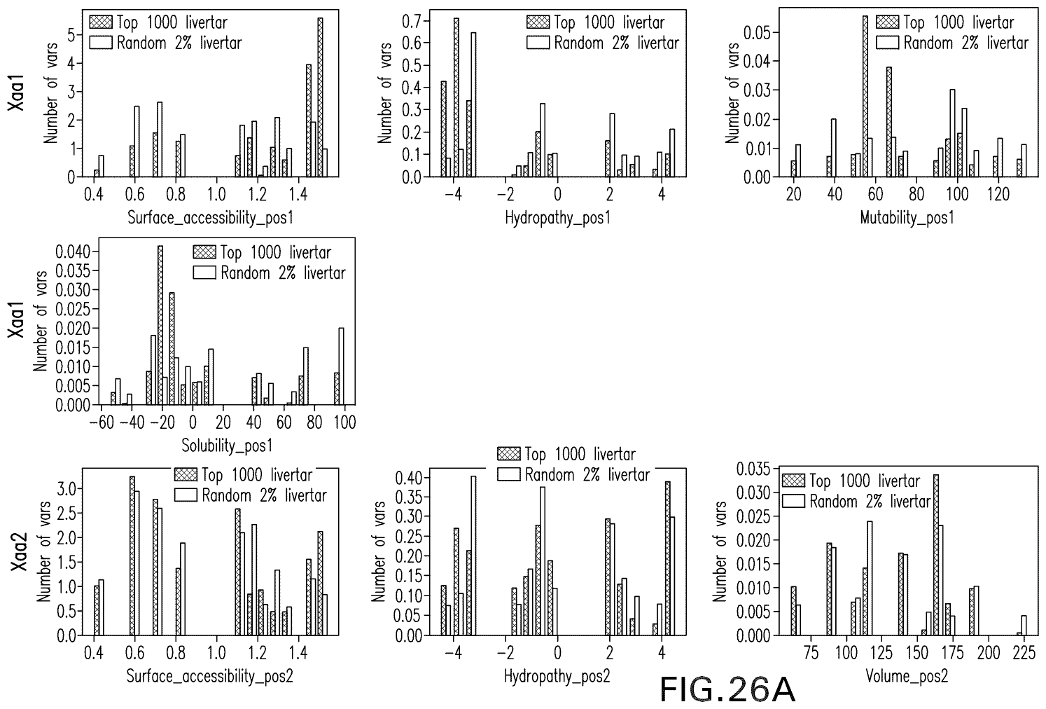

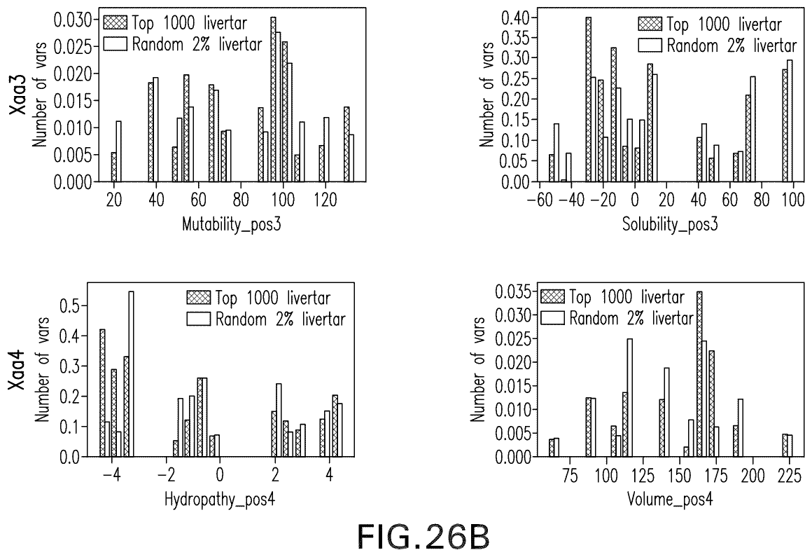

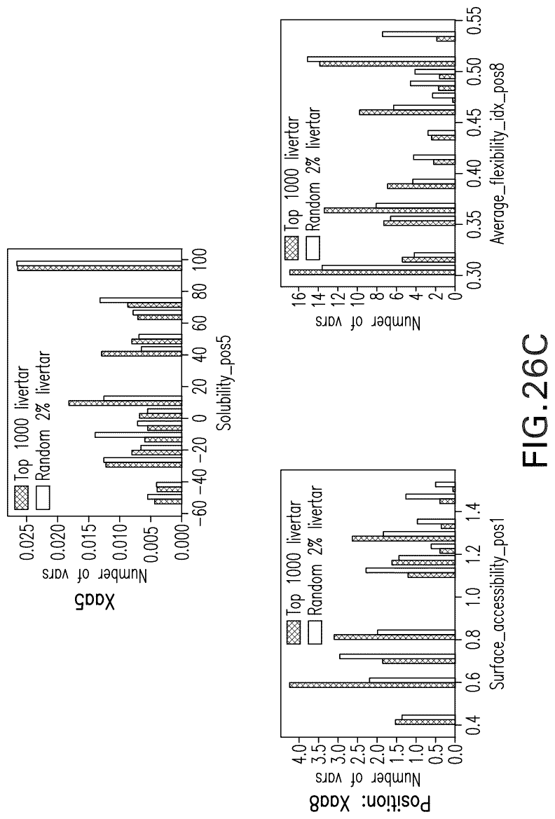

This example describes preferred properties of engineered AAV5 variants that display liver tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display liver tissue tropism. FIG. 25 shows a set of top 20 positional features contributing to model output probability. Shapley Additive Explanations (SHAP) values can be used to interrogate the relative contribution of features to model predictions. As shown in FIGS. 26A-C, these features were further compared between tissue targeting and non-targeting variants. FIGS. 26A-C show a comparison of top predictive features positionally. Features were selected if they were found to be important to both the HGB & RF models. Summaries are shown of the features in the top 1000 ML-predicted liver variants compared to random 2% liver variants.

Favored biophysical properties and favored amino acid residues at each position in the 581 to 589 region of an AAV5 VP capsid polypeptide, which are associated with a higher probability of liver tissue tropism are described below. Any of the below machine learning-derived positional amino acid preferences described in TABLE 40 can be present alone or in combination with each other or in combination with any of the amino acid preferences observed and described in EXAMPLE 4, TABLE 6. Listed below in TABLE 40 are a summary of positional features shared between the top 30 important features for liver tropism extracted from two ML models (HGB & RF).

TABLE 40
Machine Learning-Derived Liver Tissue Tropism Rules

High surface accessibility at position Xaa1
Xaa1 is selected from K, R, or E
Low hydropathy (<−3.5) at position Xaa1
Xaa1 is selected from K or R
Low amino acid mutability at position Xaa1
Xaa1 is selected from H, P, K, or R
Low amino acid solubility at position Xaa1
Xaa1 is selected from Q, K, or R
High surface accessibility at position Xaa2
Xaa2 is selected from E, R, or K
Low hydropathy at position Xaa2
Xaa2 is selected from K orR
High amino acid volume at position Xaa2
Xaa2 is selected from S, L, I, A, R, or K
High mutability at position Xaa3
Xaa3 is selected from N, I, A, M, E, or D
Low solubility at position Xaa3
Xaa3 is selected from N, K, R, or E
Low hydropathy at position Xaa4
Xaa4 is selected from K or R
High amino acid volume at position Xaa4
Xaa4 is selected from K, R, I, or L
Medium amino acid solubility at position Xaa5
Xaa5 is selected from H or T
Low surface accessibility at position Xaa8
Xaa8 is selected from V or C
Low average flexibility index at position Xaa8
Xaa8 is selected from W, V, M, A, F, L, H, or C

TABLE 41 below provides sequences of the 581 to 589 region for variant AAV VP1 capsid polypeptides that exhibited liver tissue tropism and comport to one or more of the rules provided in TABLE 40. The present disclosure, thus, provides for rAAVs composed of engineered AAV5 VP1 capsid polypeptides having a SEQ ID NO: 2, wherein the Xaa1 to Xaa9 region is any one of SEQ ID NO: 44438-SEQ ID NO: 45437, as disclosed in TABLE 41. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 41
Sequences of the 581 to 589 Region in AAV5 VP1
Capsid Polypeptide that Drive Liver
Tissue Tropism

| SEQ ID | 581-589 |
|---|---|
| NO | Sequence |
| 44438 | KAVAGCDFW |
| 44439 | KVQSAVEVT |
| 44440 | TTKPKQSCM |
| 44441 | QATKLQDVA |
| 44442 | RARHTEECA |
| 44443 | TSHKQSDCT |
| 44444 | KSGHTWDMS |
| 44445 | RVVTTSNCT |
| 44446 | VAFPMKWVC |
| 44447 | KDTFTKQWQ |
| 44448 | KAQSDCDFY |
| 44449 | KAVSSLDGV |
| 44450 | LVGRCYDLH |
| 44451 | KDGKCVSCG |
| 44452 | KCEVKQCDH |
| 44453 | SQVIRLACY |
| 44454 | ACNVCAHIR |
| 44455 | TTYKEMVSG |
| 44456 | AAMKLGNLA |
| 44457 | GSNRHMIYA |
| 44458 | AISKFIEGM |
| 44459 | ECGRLYEHE |
| 44460 | KADQFSRDR |
| 44461 | AAYKAQVLR |
| 44462 | ASWLDMCMR |
| 44463 | KYEHTTSPG |
| 44464 | QGLKCEQFE |
| 44465 | TKNTIVKAE |
| 44466 | RSNLLLQHM |
| 44467 | EKRMRAECE |
| 44468 | AIKMTNRCR |
| 44469 | TMAKYNEME |
| 44470 | RAERWGGCV |
| 44471 | NAKLEHAVN |
| 44472 | IMTKVHHVT |
| 44473 | QKRHCQTCT |
| 44474 | TKRDCNTQI |
| 44475 | RGEQKVYCS |
| 44476 | SRRLTTAHY |
| 44477 | SKKLTQQHM |
| 44478 | MGFRTSQYV |
| 44479 | AASRGNDCN |
| 44480 | KYETENGIN |
| 44481 | KAKLETWWD |
| 44482 | GMTKNTMGH |
| 44483 | ARSFVEKCR |
| 44484 | FGKYFWNSF |
| 44485 | VMVKWMRCN |
| 44486 | IVHKCIDIG |
| 44487 | VRQMNLLCR |
| 44488 | KAQYGFAVP |
| 44489 | KSEFVQSNF |
| 44490 | FTKIHWWFT |
| 44491 | TKMQVTSST |
| 44492 | MRATSHIVS |
| 44493 | KSTFAVLWA |
| 44494 | GATFCWNCG |
| 44495 | VKTQYCDMD |
| 44496 | KVGPAADFA |
| 44497 | AASFHAMDR |
| 44498 | EKVMRKPAD |
| 44499 | KVTIGTLAN |
| 44500 | AKMNYANVD |
| 44501 | SKQVCLAHE |
| 44502 | VAKMSFDCC |
| 44503 | RPSYKVKCQ |
| 44504 | ATHKIIGVE |
| 44505 | KSMHTVNEP |
| 44506 | VCARTNNLC |
| 44507 | TKVIYEKAV |
| 44508 | RSGVTYAWA |
| 44509 | RTQTFTMIP |
| 44510 | KCEMNISCH |
| 44511 | KPLQAFAFN |
| 44512 | AELRYQFYM |
| 44513 | DKRVIVKCE |
| 44514 | SNCLLGQCT |
| 44515 | QKQGFAINQ |
| 44516 | SKLLYSTSG |
| 44517 | KITFCWPCH |
| 44518 | RMQYTQSCS |
| 44519 | GTRTSFAMD |
| 44520 | VVKQTTGMD |
| 44521 | SATRAFEVQ |
| 44522 | SNGLQNIAF |
| 44523 | KMQSRLYCY |
| 44524 | TIKPSWLCA |
| 44525 | VSVRMHEMH |
| 44526 | KAMMDHAYD |
| 44527 | LASRAENHS |
| 44528 | KMQVDNANE |
| 44529 | MCMKAQRSG |
| 44530 | TAMTDVRDR |
| 44531 | RINRNNCRI |
| 44532 | MAQLLYSCQ |
| 44533 | QCRRMEGVE |
| 44534 | AMQRALINA |
| 44535 | DRKTQMIST |
| 44536 | NGFIQYDSK |
| 44537 | SKNAANSWD |
| 44538 | QKKFMECAQ |
| 44539 | SLFLEKSCG |
| 44540 | KVLECHGAA |
| 44541 | KIESTSSHC |
| 44542 | KHQFTGLHS |
| 44543 | QRKMNPDCM |
| 44544 | KMSTCMEHN |
| 44545 | AIGRNDQTT |
| 44546 | NAVVMMSNK |
| 44547 | RNIQGISWA |
| 44548 | AKSPITNGM |
| 44549 | SSNRYGDVT |
| 44550 | MRSGFQMST |
| 44551 | IAAEFKACR |
| 44552 | KLNSTMYVG |
| 44553 | LAVRTKQFH |
| 44554 | KQTSPLHIG |
| 44555 | KVANFFDEP |
| 44556 | TKQHFPLGE |
| 44557 | MIVKIERGV |
| 44558 | ACSKLTCCY |
| 44559 | LMYKMHQIL |
| 44560 | TAGKTWEGK |
| 44561 | KFHNSIVVT |
| 44562 | AACRISEVP |
| 44563 | ISTKAHQME |
| 44564 | SQKSHTVWA |
| 44565 | VKFLVQCHD |
| 44566 | SVTIWENFV |
| 44567 | KANPSNGFQ |
| 44568 | TTGWQVQCM |
| 44569 | SYVFAQIGC |
| 44570 | ATRQLFDCR |
| 44571 | GTHRTEMGR |
| 44572 | AQNRIGNQN |
| 44573 | MSNTAVQFV |
| 44574 | QKASYVEFD |
| 44575 | FGMKSKQMS |
| 44576 | IQREVTWCT |
| 44577 | NVNREHRFA |
| 44578 | MDRLLIKMA |
| 44579 | TRFMTDCMF |
| 44580 | VKGQMYSCE |
| 44581 | KCEEQKPCP |
| 44582 | VCLQFYQGR |
| 44583 | MTRASHVMC |
| 44584 | RADQQVRWA |
| 44585 | RADRCSDLY |
| 44586 | TSVTIVECV |
| 44587 | SNRTCAVSV |
| 44588 | VRTYVQACE |
| 44589 | ALMKLWCAM |
| 44590 | QKNPTLDSA |
| 44591 | SMRRNKYWC |
| 44592 | TKAEHVVMH |
| 44593 | VAVKHHTTC |
| 44594 | FCKTSTSFN |
| 44595 | SANLLSDFH |
| 44596 | KSVMDDVVD |
| 44597 | KGETTQNYG |
| 44598 | KAMDGAALT |
| 44599 | KMEVRMDHG |
| 44600 | TKTTTNISI |
| 44601 | TIAREKGYP |
| 44602 | AIKPSTWQQ |
| 44603 | MKFLLELWS |
| 44604 | KGMMKDTEQ |
| 44605 | IMKPFMEMW |
| 44606 | ACVTCTTFK |
| 44607 | AMKSRCRCE |
| 44608 | MCKFSVWAL |
| 44609 | YNKAYGYMA |
| 44610 | QRAHYIDCD |
| 44611 | LAAVWVVDG |
| 44612 | MAHGFQRWC |
| 44613 | STKIQYFCE |
| 44614 | EYVAWMKQW |
| 44615 | KSVQKECYF |
| 44616 | MCAQWCQCN |
| 44617 | RAEAHWHNF |
| 44618 | NKLIQEGCP |
| 44619 | EKAGSGAWN |
| 44620 | SAGMKPMCC |
| 44621 | RKSQKRMIN |
| 44622 | SAAIDRLQR |
| 44623 | AQAYYGVCD |
| 44624 | MAMRVGSHQ |
| 44625 | QKTPTMNEW |
| 44626 | NCTMIQRNY |
| 44627 | KCNSQPSSD |
| 44628 | IKQPYALTA |
| 44629 | GQQLIARCG |
| 44630 | EERPWYCKE |
| 44631 | NRELRMHHE |
| 44632 | TEQIFMKGC |
| 44633 | QKRLGEEVE |
| 44634 | TTYKAMSGD |
| 44635 | SHMFQYSHT |
| 44636 | TLKVHGIGG |
| 44637 | KIQQNMETL |
| 44638 | KAMQPECGA |
| 44639 | ASYRMGDHP |
| 44640 | AFAKLIHTE |
| 44641 | KAEPNMALE |
| 44642 | FRKSYWYTG |
| 44643 | MGGGQASWY |
| 44644 | VLHKAEICL |
| 44645 | YCTQCRGGV |
| 44646 | MCSQCAVLD |
| 44647 | RVNTSSRAT |
| 44648 | IRYAYDGNE |
| 44649 | VATQLIHSY |
| 44650 | IWKQLVEMQ |
| 44651 | KGGTENGWN |
| 44652 | MSQRIWTVG |
| 44653 | NKQMFCKGP |
| 44654 | MTYRSEFMR |
| 44655 | APGKGNRAC |
| 44656 | MFTRFMRCG |
| 44657 | RCMQSFTFH |
| 44658 | TIFRLRKSC |
| 44659 | MMSSQMKCG |
| 44660 | HTARNVRMR |
| 44661 | TGKSWTMLG |
| 44662 | EAAKESRLA |
| 44663 | GVKLQSYMT |
| 44664 | DRRYFTYAG |
| 44665 | LCYMLFEMT |
| 44666 | TIKCWNWSN |
| 44667 | STGMWQCAQ |
| 44668 | KLTECPLCG |
| 44669 | KTFFYHAPC |
| 44670 | RISDAYQCP |
| 44671 | SSKYAHYCA |
| 44672 | IATVWARCP |
| 44673 | TIGRTQFCH |
| 44674 | SSHMEFSAK |
| 44675 | MIQRVPTVA |
| 44676 | SKTDKMEFY |
| 44677 | TTAIRMYCV |
| 44678 | KCQYCSDMY |
| 44679 | TCKPIYHIE |
| 44680 | RVNRKQINI |
| 44681 | SGGKSQIVP |
| 44682 | MFRINTECC |
| 44683 | NAATIYREA |
| 44684 | ASTKQEYWR |
| 44685 | TSALKPACN |
| 44686 | TVNLNGESR |
| 44687 | SGTFAPFGW |
| 44688 | ACAKDRYAS |
| 44689 | KCMSALEQQ |
| 44690 | ALTMLIKGC |
| 44691 | SKTLFHMIQ |
| 44692 | VSVIKFKGE |
| 44693 | KPAEAHSVW |
| 44694 | TRQLYGLHD |
| 44695 | KKVMGMLYH |
| 44696 | MKTPLAEAH |
| 44697 | KVEGCKSHY |
| 44698 | HVAMCSNLM |
| 44699 | KKDTVNENA |
| 44700 | LSGRMAQQA |
| 44701 | ATLVKAFCA |
| 44702 | ACTHLMCRP |
| 44703 | QVTKLPPFS |
| 44704 | SCHMIAEFA |
| 44705 | SGYFRIQLS |
| 44706 | STKIGAKFL |
| 44707 | TSNFNCKMW |
| 44708 | GSQRIGIIP |
| 44709 | QRAGSFGWD |
| 44710 | NMRMWEWTT |
| 44711 | IAVKAWFDH |
| 44712 | KSNKRCGHL |
| 44713 | MGLQRMEFC |
| 44714 | KPSHYSCAF |
| 44715 | KGATDVPSG |
| 44716 | LAIKCMDFW |
| 44717 | ADNFLVKHG |
| 44718 | SRVIRSDSC |
| 44719 | MKQFCTCMS |
| 44720 | NCLLQWGQC |
| 44721 | GAAVLSALH |
| 44722 | KKNTAFAST |
| 44723 | QRTATLEAW |
| 44724 | SKLTVAAVE |
| 44725 | SMYRVMPCV |
| 44726 | HGILQNCFI |
| 44727 | LTVQLLECA |
| 44728 | MMMRQHLYG |
| 44729 | TAHFSFDTA |
| 44730 | QKKLWYGPV |
| 44731 | VGHKLSGNC |
| 44732 | TKLQFTSPH |
| 44733 | VGNFLYRAY |
| 44734 | TSGTSYQCQ |
| 44735 | RWQNAYEVS |
| 44736 | ARVMFDMSR |
| 44737 | SGQFFNVSL |
| 44738 | VSMMLCASL |
| 44739 | KGGLQENFG |
| 44740 | TALAKLHVG |
| 44741 | ACQPSFAGR |
| 44742 | ADKPLYQNE |
| 44743 | DWVLWTRIC |
| 44744 | VANAHWCVG |
| 44745 | SCAKTFAYS |
| 44746 | KIQQDIDMG |
| 44747 | KLKTQELWS |
| 44748 | IVMREAQAS |
| 44749 | TKNFTKGAW |
| 44750 | LCAKEGSHE |
| 44751 | TCLHVWQCG |
| 44752 | TSHVMYQYC |
| 44753 | AAVMQFMGV |
| 44754 | KMDWPNVLN |
| 44755 | TNQRIPNCE |
| 44756 | RSKDGGNCW |
| 44757 | SHTIFLHSF |
| 44758 | TGRLQEAYA |
| 44759 | SFCFCSANF |
| 44760 | TASFFMSVM |
| 44761 | ACQRVEKYT |
| 44762 | RAIAGGECV |
| 44763 | VAVKGWWGT |
| 44764 | KRCVTHECR |
| 44765 | TAYMTFDQA |
| 44766 | SSYRGVACE |
| 44767 | FCGSCVAFE |
| 44768 | AMGINMSGF |
| 44769 | KVVPNFPAQ |
| 44770 | TCSKDGSWW |
| 44771 | ERSPTFESQ |
| 44772 | LKASYHYQR |
| 44773 | FANTLANNA |
| 44774 | ATRQDGMAY |
| 44775 | SPASCQAAA |
| 44776 | AMGFSPHER |
| 44777 | SRYVVNSAH |
| 44778 | MANIWHKGA |
| 44779 | TKTPCWVWS |
| 44780 | AWGSFIHMR |
| 44781 | VYNFSARER |
| 44782 | VIAKMCEQN |
| 44783 | NCNLYYDHL |
| 44784 | TIFTTYSMK |
| 44785 | LAGKAQMWQ |
| 44786 | KMNGCQSTE |
| 44787 | MKQRTWSGK |
| 44788 | MMVVWQNMC |
| 44789 | FCVYFVDGK |
| 44790 | SAMIAGLGK |
| 44791 | DRKLFQFSG |
| 44792 | TSWTCPRFG |
| 44793 | MMKILQHSA |
| 44794 | QIVVWFKEP |
| 44795 | VSRITTIMA |
| 44796 | SAGSCAHMQ |
| 44797 | LSLRLERMC |
| 44798 | KGTPNLVSE |
| 44799 | TMFSCKYIG |
| 44800 | VTNMRLEGV |
| 44801 | AVKLPMCVD |
| 44802 | KINLTQSAT |
| 44803 | SAQTHIKFM |
| 44804 | TAVIRSNTA |
| 44805 | VFNRLIQSI |
| 44806 | QKAHIFDVT |
| 44807 | NSSKLIKYT |
| 44808 | GAGKSNCNP |
| 44809 | RGDTQHCMY |
| 44810 | KKEYNNGWW |
| 44811 | AAYQMTGIY |
| 44812 | TAMHTHKLG |
| 44813 | NHNMCAVVV |
| 44814 | DNRFMFVDT |
| 44815 | KWAENTVSG |
| 44816 | HALLKMCEG |
| 44817 | ISSTCKFGQ |
| 44818 | TRLKLDVWS |
| 44819 | SAYQLTGTA |
| 44820 | ACVASIGMK |
| 44821 | FCRIHMMCW |
| 44822 | QCRKDWGHY |
| 44823 | FCNTLVISA |
| 44824 | AWYRTEQMW |
| 44825 | TMKSWVHGC |
| 44826 | SAAGMNDCL |
| 44827 | KTTTCWADP |
| 44828 | ARRIEEGYG |
| 44829 | LCMPLLTFD |
| 44830 | NGNVVMKEY |
| 44831 | SSLTCSHSV |
| 44832 | QGLKIYCHP |
| 44833 | AGVGSWQNE |
| 44834 | KGAIQQEWW |
| 44835 | AKNPYLWYQ |
| 44836 | ACSILYRHL |
| 44837 | NTWMNLESC |
| 44838 | TGGQLFQCW |
| 44839 | MNVKKSGSL |
| 44840 | MGLPVADWG |
| 44841 | FSTSHFHLW |
| 44842 | GSVKGMSVS |
| 44843 | YSGMFHEGR |
| 44844 | GWKPYAYAE |
| 44845 | ACCLTMCSY |
| 44846 | STRTTSSSM |
| 44847 | AAVPCSMQQ |
| 44848 | STQMQTVGL |
| 44849 | NFFLTAVEH |
| 44850 | YAQSKVRMG |
| 44851 | AGGKAQAVE |
| 44852 | TATLCANFC |
| 44853 | TGTVKMGME |
| 44854 | TTYIRLGGP |
| 44855 | SVFLSQSCR |
| 44856 | AKYEKVGMA |
| 44857 | ITQIWARVI |
| 44858 | QMNRLGNWN |
| 44859 | QSSWHSKVW |
| 44860 | KSDQSVCDQ |
| 44861 | LAASCGHVG |
| 44862 | GATTCKQID |
| 44863 | YKNHFMHMW |
| 44864 | VALVVVMSH |
| 44865 | ACVTNLWGD |
| 44866 | MMMRQEFCE |
| 44867 | IRKYYHNGV |
| 44868 | KFAIAQPVS |
| 44869 | TAMKQPTLP |
| 44870 | SVIIHPSYE |
| 44871 | VGAQFAVQF |
| 44872 | SKNELFVMH |
| 44873 | TACFNALSC |
| 44874 | DCYKSIVGV |
| 44875 | VKFLVQQGQ |
| 44876 | GTQTRFDNS |
| 44877 | KSGPVVCKI |
| 44878 | VAHISSGAL |
| 44879 | ARNLAQGGY |
| 44880 | VFGINGKCY |
| 44881 | FFTQCARVP |
| 44882 | TSWFKDCVG |
| 44883 | STQIWSKMH |
| 44884 | ISGTNRMVP |
| 44885 | GAHTCAKAG |
| 44886 | TIVFSLHRD |
| 44887 | MCAQMMGVE |
| 44888 | TSASCCQLT |
| 44889 | KGRWVDPMA |
| 44890 | MWKEITGQS |
| 44891 | SALITNADQ |
| 44892 | TCTTLTNQA |
| 44893 | SSVHQMLTC |
| 44894 | AGKITWQND |
| 44895 | SGKTHSGEQ |
| 44896 | MGHMCPGCF |
| 44897 | TSTLHFSAD |
| 44898 | KYETENGIK |
| 44899 | RASVTMGSA |
| 44900 | VTQKCREYW |
| 44901 | FTTVCAGCV |
| 44902 | MRVTYPWAD |
| 44903 | AGAFMMPNW |
| 44904 | SKSDKLECR |
| 44905 | GGTYKLNGC |
| 44906 | KTIPVCAHS |
| 44907 | KADQGVKDW |
| 44908 | KSTPLAEGV |
| 44909 | VGKGMNRFN |
| 44910 | KINTECMHD |
| 44911 | MAIIMSELG |
| 44912 | TPKHQQGYY |
| 44913 | FREEYRRGG |
| 44914 | ACNYFSREC |
| 44915 | ATLKVMGHH |
| 44916 | KCTTEGTNA |
| 44917 | AMLMNTACG |
| 44918 | TINRWMVAF |
| 44919 | NGNMIWRGK |
| 44920 | IVNTFWNYC |
| 44921 | SVYLKPTVS |
| 44922 | KHALAVSQP |
| 44923 | VGNQIPRGE |
| 44924 | STHILEGWD |
| 44925 | KSVGGTEHP |
| 44926 | QKNATAVNN |
| 44927 | FCSQHLTGP |
| 44928 | QCRVCQFHA |
| 44929 | VAAQFSRFE |
| 44930 | RSSSETLWS |
| 44931 | HKKEFVMNS |
| 44932 | TKNPTQLER |
| 44933 | TGMVLNGAM |
| 44934 | MKSHQMWVM |
| 44935 | VPKYWQQAC |
| 44936 | LVWRLHQAP |
| 44937 | KSTAPEMMN |
| 44938 | SFVIHSRTA |
| 44939 | SMFIFLHIC |
| 44940 | AMASFWECG |
| 44941 | QSFIMYGDC |
| 44942 | ASLKSMEYR |
| 44943 | VGMANMTCQ |
| 44944 | MCGHHAQLG |
| 44945 | ISQLVWKCV |
| 44946 | TAYRAIGPY |
| 44947 | QRGTYNQMM |
| 44948 | ATVGKVDIQ |
| 44949 | KTATNMNSF |
| 44950 | GCGLWVYSS |
| 44951 | VTTSLMDSA |
| 44952 | KSMMNESAV |
| 44953 | TIIACPRGS |
| 44954 | AAFGMNTFP |
| 44955 | VLYKIWQEK |
| 44956 | TCRRYECSV |
| 44957 | EKAMKSCTF |
| 44958 | AQNLHFYAP |
| 44959 | AKSQCSKGL |
| 44960 | VGTPTCQGV |
| 44961 | GSHSHMLDR |
| 44962 | SAAYRYCEP |
| 44963 | KVNPLNGHW |
| 44964 | GRIQFIMCG |
| 44965 | KATQEVWHK |
| 44966 | KKEAVGIMN |
| 44967 | GASSSYQNS |
| 44968 | EREAHCNQA |
| 44969 | VPHISFKCC |
| 44970 | LAHMAYPSV |
| 44971 | TALVQYNYP |
| 44972 | ESMRGQQWW |
| 44973 | TVAFSMCRP |
| 44974 | RCAMDIPHG |
| 44975 | LAMPCSGLC |
| 44976 | GKQFNMGDP |
| 44977 | NKTLVGIQS |
| 44978 | TANIASVPG |
| 44979 | MIVKTVMDV |
| 44980 | VTWSCSEIA |
| 44981 | IGHGVQTAF |
| 44982 | KCNNHVNSS |
| 44983 | TRVPTQSIF |
| 44984 | TCHVSQDCT |
| 44985 | NTFMSTFLE |
| 44986 | TYQFRNRSE |
| 44987 | SAAWQKRCP |
| 44988 | RKEEFHACN |
| 44989 | GSMFTCYLA |
| 44990 | GESRLNEHW |
| 44991 | IKTPYCSSG |
| 44992 | FANSTQGCA |
| 44993 | AACLNTQYR |
| 44994 | FISFHEGYR |
| 44995 | HQMKAMNLG |
| 44996 | ACMGIKHVH |
| 44997 | IAQQTWQFE |
| 44998 | ERYMFLNME |
| 44999 | HKTPYPHLR |
| 45000 | RAEMLHEAV |
| 45001 | SGQHMPGCM |
| 45002 | GYMLKSDVQ |
| 45003 | YCVQTAFMH |
| 45004 | FRAYFQSAQ |
| 45005 | VSFTWNRCI |
| 45006 | ACASLINQM |
| 45007 | TNRHAEAYI |
| 45008 | SCQPAYGGG |
| 45009 | YTNLVIGVW |
| 45010 | MIFVISVGN |
| 45011 | TFYVQVNDK |
| 45012 | SANKVVHTD |
| 45013 | KCAMALIHK |
| 45014 | MLFWFRCLL |
| 45015 | DIEKLHHVF |
| 45016 | RYVCPLDHY |
| 45017 | SCCQEANGA |
| 45018 | THYFRNMHG |
| 45019 | AFVPFGNDL |
| 45020 | LGKVVYQAY |
| 45021 | ASKLDTALT |
| 45022 | MCKLLATSQ |
| 45023 | SLKAEYSGN |
| 45024 | NTQTVNRMF |
| 45025 | QVHKGWACV |
| 45026 | SQRVWEQVC |
| 45027 | THTKAQIGH |
| 45028 | MGSRHFHFP |
| 45029 | GHYVCSMAY |
| 45030 | DKHGNANWN |
| 45031 | GMSRQSIAD |
| 45032 | KAEHFNVDA |
| 45033 | IKMNHHIQG |
| 45034 | IFGKQHRLH |
| 45035 | ATGTKISVE |
| 45036 | MAKINPLHG |
| 45037 | TTRHCAYAQ |
| 45038 | MIQFDMMHG |
| 45039 | KTKSGAVFT |
| 45040 | AQKLMMGQG |
| 45041 | VSNLTWPGP |
| 45042 | ESKIVRYNN |
| 45043 | SAGRWCQTY |
| 45044 | NGALQMVQW |
| 45045 | GMFIQAVEA |
| 45046 | AGNTTIKFE |
| 45047 | AYFSCMHVN |
| 45048 | LGMTCVAHG |
| 45049 | VAILALRCH |
| 45050 | TKQQVGDCW |
| 45051 | TGLMLQQIC |
| 45052 | NVHFLFNCA |
| 45053 | NYRVAEWVA |
| 45054 | AARIACFDS |
| 45055 | VCVTKQMAC |
| 45056 | DRRPVCCYT |
| 45057 | KNDKGQCYF |
| 45058 | LGIKSQDAA |
| 45059 | QSALMSKYW |
| 45060 | TVGIKFQEM |
| 45061 | YTKFMSGGM |
| 45062 | AFRSPNVMY |
| 45063 | YSSLLALRA |
| 45064 | TQAKLQCAE |
| 45065 | TANHATMCD |
| 45066 | SPQPILDLQ |
| 45067 | TTVLIQICE |
| 45068 | SAASLIPTL |
| 45069 | MKVDSTFIH |
| 45070 | LGAANQMCH |
| 45071 | MSTADMIRI |
| 45072 | KAALMGWEK |
| 45073 | NCVVLQKLH |
| 45074 | DRAPKLSWE |
| 45075 | VCNQFQHWH |
| 45076 | KISFEVTEY |
| 45077 | TFYIWNHCD |
| 45078 | MCKQIIQGG |
| 45079 | SCSVWRCMS |
| 45080 | VSILMYQNQ |
| 45081 | TSMFYEMCN |
| 45082 | SYKHCSQVG |
| 45083 | FAAQWFWTG |
| 45084 | TWATWLEGQ |
| 45085 | SAQVCQFIQ |
| 45086 | NAMSQVSLA |
| 45087 | SNQFFPQSG |
| 45088 | TGTLMGSQG |
| 45089 | MCNYSNSGE |
| 45090 | TCSQPPCCG |
| 45091 | GHYIFNCSQ |
| 45092 | IGHRYYWSM |
| 45093 | GCHTIQFYR |
| 45094 | NCNMYHYFA |
| 45095 | GAQPRVVLS |
| 45096 | KATCYMDAN |
| 45097 | SAKQWTTVE |
| 45098 | NANTNGHMF |
| 45099 | TAGQPGILG |
| 45100 | WGTYWFNMP |
| 45101 | VSSKSVHDV |
| 45102 | SPQSFPQFE |
| 45103 | SKAANNQCC |
| 45104 | MGYQPAVGN |
| 45105 | AEMRTMIQA |
| 45106 | GGTFIQGQG |
| 45107 | KGTDDGQWQ |
| 45108 | GCNIQNMAA |
| 45109 | AGMGIHAMP |
| 45110 | AGQLVQACH |
| 45111 | TRDTFFKMG |
| 45112 | STYVQMDGG |
| 45113 | TSTTKMLSC |
| 45114 | TANATVFHS |
| 45115 | TCEQEWKAR |
| 45116 | SAMPGASMN |
| 45117 | GASYANNVV |
| 45118 | AAFTHMGTF |
| 45119 | AVKNWEVHE |
| 45120 | VASFWHCGA |
| 45121 | AIYPTNFVK |
| 45122 | NPALHNQCQ |
| 45123 | QKMAVCDMP |
| 45124 | KLHQPTWAA |
| 45125 | SAKYHMDVE |
| 45126 | QCVANYIAR |
| 45127 | GWHVQSCAM |
| 45128 | VPITCYNHG |
| 45129 | DKQPHNTGF |
| 45130 | AQNGMAICE |
| 45131 | VLFQTMVED |
| 45132 | TRKAVCQPV |
| 45133 | VALKMFCTY |
| 45134 | GANTEVLAG |
| 45135 | MGAVTMVGS |
| 45136 | MSYLKTTCH |
| 45137 | TMGLHQDHP |
| 45138 | LMHPLGTCA |
| 45139 | TMGLAGKSA |
| 45140 | YGQHWNPLT |
| 45141 | ACYQLPSAN |
| 45142 | ATKEKHPFY |
| 45143 | MMKNTQFCE |
| 45144 | KTNFRCEYY |
| 45145 | MVAKEIDGP |
| 45146 | MGSLQNMEF |
| 45147 | RAQPQERVV |
| 45148 | TKAECMPYQ |
| 45149 | VAMFNRLDR |
| 45150 | KAHDVLSNP |
| 45151 | VTNLLMRAS |
| 45152 | VGWACQAFY |
| 45153 | MTNSSHKWS |
| 45154 | KAAWSEDGR |
| 45155 | ASGLYYSHG |
| 45156 | MTGLQRVAV |
| 45157 | KPSTKMTLR |
| 45158 | IATKIRGFY |
| 45159 | ARGVLNLED |
| 45160 | FMNMMGSCS |
| 45161 | FAHQHPGCP |
| 45162 | NCVSLAAHW |
| 45163 | DSMRCTPVP |
| 45164 | FDRLITQGE |
| 45165 | TGLADQDWW |
| 45166 | NSIASKKSM |
| 45167 | MFYYAFTCG |
| 45168 | NSMVGGMSE |
| 45169 | LVIPWARVE |
| 45170 | FAAMACDMT |
| 45171 | NSTSIFDAC |
| 45172 | IAAHVVMCD |
| 45173 | SAVPVCSCI |
| 45174 | NKNSTNQWE |
| 45175 | TRFEYWSNS |
| 45176 | QMTMWANFY |
| 45177 | SPNPLPWCD |
| 45178 | KAINPLKAT |
| 45179 | SVSIFKGSN |
| 45180 | GNYISSMAC |
| 45181 | YATTTMSML |
| 45182 | KVHTCGMNY |
| 45183 | NWNMWLKSL |
| 45184 | FAAITRFQS |
| 45185 | SVTAKNLCL |
| 45186 | VCTICPVQM |
| 45187 | NIKLTHFEM |
| 45188 | VTQISCVDT |
| 45189 | GAGMEASMD |
| 45190 | AAVSVHKCL |
| 45191 | TAALWKIEC |
| 45192 | TGQPYYNTA |
| 45193 | AILPCSTPK |
| 45194 | EKQVYIVRN |
| 45195 | MAKPAHAED |
| 45196 | KVHEASYFP |
| 45197 | VMIKTWASL |
| 45198 | HSATAVREG |
| 45199 | GQIFMCQHI |
| 45200 | AVSLHEHLQ |
| 45201 | AAMPVMKCW |
| 45202 | LGAMMALHC |
| 45203 | RCGLSSDAT |
| 45204 | LALQIQENR |
| 45205 | TARTPNDFE |
| 45206 | MGTFKLDFN |
| 45207 | TSTRANMLC |
| 45208 | AFKLVSCLE |
| 45209 | ISLPCHEVK |
| 45210 | EKGYIFVID |
| 45211 | TTSVLTGTG |
| 45212 | FARQFMEYW |
| 45213 | FGHFVGLTG |
| 45214 | STMSIVRTP |
| 45215 | ASVTNVKGM |
| 45216 | MSKLHAGIH |
| 45217 | GIAFAVHGI |
| 45218 | MFQTTILES |
| 45219 | MAHSYQSFC |
| 45220 | VTQMIANVQ |
| 45221 | DRFTQQSAK |
| 45222 | VQKYVTQAF |
| 45223 | SASTTRFIG |
| 45224 | VAATAYMDE |
| 45225 | RGEHFKCSY |
| 45226 | HSNMWLDQY |
| 45227 | NKTSNIMYH |
| 45228 | AAYSPMFWR |
| 45229 | SVMPCVDAY |
| 45230 | TAVPILDWT |
| 45231 | SGMLFIYSG |
| 45232 | NRLTIHTWY |
| 45233 | VAVITQQYP |
| 45234 | NSRLIRPHG |
| 45235 | RTIHPMRFI |
| 45236 | SKFEFQNAA |
| 45237 | MGRPVPSHS |
| 45238 | MAHNLVSLS |
| 45239 | KMMCNPTLL |
| 45240 | NIKLTHCNF |
| 45241 | AASSWVLME |
| 45242 | MCLQPYNCY |
| 45243 | VTHSTPQCV |
| 45244 | TSKVVSDAP |
| 45245 | ASSIVFTAA |
| 45246 | KLHSGGYLC |
| 45247 | TAGIIYFYH |
| 45248 | SGKQLQNTY |
| 45249 | RFTMYSEWL |
| 45250 | TSRPISHEI |
| 45251 | SGMLKPMYS |
| 45252 | RTTMGVKME |
| 45253 | TCLTRHSQG |
| 45254 | MADKKWRYC |
| 45255 | SFYIHQEAN |
| 45256 | ASYMKSELA |
| 45257 | SYTRAVECN |
| 45258 | SVQMEGLGW |
| 45259 | GTKHTMQQC |
| 45260 | TSQHLQVLC |
| 45261 | MHNRIQKGG |
| 45262 | VIKSYVSTA |
| 45263 | RLQYAGQFL |
| 45264 | DKQFIAEWP |
| 45265 | SHEHCMKMC |
| 45266 | KTEDYQHHQ |
| 45267 | AVYMAGRYM |
| 45268 | GATPLCDAN |
| 45269 | GAYHVTNQT |
| 45270 | SHQHHPWGA |
| 45271 | GATIVMTGH |
| 45272 | TAVAQVDPV |
| 45273 | TARNLMGSS |
| 45274 | KGNHKMKHE |
| 45275 | RIEDTRHNN |
| 45276 | MCHQWMSCT |
| 45277 | STHYKIWAG |
| 45278 | TMKAMMEVP |
| 45279 | ALAHEAGKR |
| 45280 | SSNIIQYHE |
| 45281 | TQKSGSFNN |
| 45282 | ALKLAQSHY |
| 45283 | VCSHFCNEQ |
| 45284 | MSAWKMRDR |
| 45285 | TVTVWPVCC |
| 45286 | NSKHLNTYV |
| 45287 | VSHWITKVR |
| 45288 | TSMPYKAYG |
| 45289 | VYLQHNCCW |
| 45290 | AANVGMLGS |
| 45291 | KLQMNYEEK |
| 45292 | LGGFHVSAS |
| 45293 | GATFLQFDG |
| 45294 | SQMLSEHLE |
| 45295 | TPGWVNQIA |
| 45296 | TGTFVQMDK |
| 45297 | ATKQTPWAI |
| 45298 | TMCHEMCSL |
| 45299 | VGAACKMVA |
| 45300 | GQVTMQRCC |
| 45301 | TWTIKQIGE |
| 45302 | SARQVLDPD |
| 45303 | VSKSWLNSF |
| 45304 | GGMFLAEGS |
| 45305 | NYVTLGNHM |
| 45306 | ISKHCHDQG |
| 45307 | SAQSALDAL |
| 45308 | FAHGHSKAR |
| 45309 | AYFYLGAEF |
| 45310 | QKTQHPVWN |
| 45311 | NLCVSLESY |
| 45312 | MSQRVSVPS |
| 45313 | KEKLQMNHM |
| 45314 | HKGFTALFQ |
| 45315 | GTRTRIQQE |
| 45316 | ACTNMFAYD |
| 45317 | QSMHCANLK |
| 45318 | LGSHYCNYA |
| 45319 | VAMLEFQSA |
| 45320 | SSHMLELHI |
| 45321 | HGVIPMNWI |
| 45322 | SGAKMAWVA |
| 45323 | SQWMLPCGE |
| 45324 | MPTHMWDAP |
| 45325 | NVMMQTYSR |
| 45326 | TCQGIGSVT |
| 45327 | TCRVAEFVP |
| 45328 | MGVMLTSEW |
| 45329 | AQNLYSAFW |
| 45330 | NHQLTYCSG |
| 45331 | MIKLRHWDV |
| 45332 | HCGPCLKGD |
| 45333 | AAQYQAIGV |
| 45334 | TNKMVGHGH |
| 45335 | RCGVQQGSN |
| 45336 | TFMRNEWTY |
| 45337 | AKYICQNYC |
| 45338 | GCARNNGAI |
| 45339 | GCGKLEIHF |
| 45340 | TAVQTHTGA |
| 45341 | ARMPRESAD |
| 45342 | VMIPRMGCG |
| 45343 | YGYMANKMK |
| 45344 | NSYTNFKGG |
| 45345 | VLYSWWHFD |
| 45346 | KCFEDLCST |
| 45347 | VSNPMSCKK |
| 45348 | SCQHFMDDP |
| 45349 | VAAHWKMSN |
| 45350 | AAVAMQMCE |
| 45351 | ISQHCHCKL |
| 45352 | QWKQAQCCT |
| 45353 | GHRSRLSGA |
| 45354 | TCASLQSNQ |
| 45355 | KSAQMYGES |
| 45356 | RSATDYGNH |
| 45357 | ASGTKWAHK |
| 45358 | SPVTLHNGE |
| 45359 | RKEFWIKFK |
| 45360 | VAARYTAPN |
| 45361 | AVKQTIYMP |
| 45362 | FKILKPGLG |
| 45363 | AAIPFACMG |
| 45364 | MCRLWHDAW |
| 45365 | AQKYILVGQ |
| 45366 | IQIFCFWCG |
| 45367 | NRKGQHVQL |
| 45368 | TKKTCVTWG |
| 45369 | TVNMWGADD |
| 45370 | GFKPEMRMA |
| 45371 | HRKHKFVPI |
| 45372 | KLSWEEYVR |
| 45373 | VIAQQGNAR |
| 45374 | TTAHVYDGM |
| 45375 | VKQMKLEQH |
| 45376 | MCVMSQEHL |
| 45377 | GRMQTSRCF |
| 45378 | TMRGTGAQH |
| 45379 | HYRPFWSAC |
| 45380 | ELFMDHRSW |
| 45381 | FSQPCVNGQ |
| 45382 | RPFTNEGLC |
| 45383 | VMSPFGKFC |
| 45384 | TVKIYGEAS |
| 45385 | AYRYEYCAI |
| 45386 | IGTTWYGDT |
| 45387 | ERARSHECT |
| 45388 | KIESIVGVT |
| 45389 | MGASNMRFV |
| 45390 | VANYCNRPE |
| 45391 | AGQTFIYVH |
| 45392 | LAMMCHADQ |
| 45393 | MHVMSGHLF |
| 45394 | AMRQANAGV |
| 45395 | QYSIWNFDH |
| 45396 | TTHLGMNHF |
| 45397 | MSLHKCDVR |
| 45398 | NFAMSMRDH |
| 45399 | MKYEKNSCW |
| 45400 | GSSYLNQPT |
| 45401 | MSRHCSMHD |
| 45402 | MANATSREN |
| 45403 | EAYRNKNVC |
| 45404 | KNNTKKQWP |
| 45405 | RVAFTLESF |
| 45406 | AYKQIHDST |
| 45407 | NATGAPRAW |
| 45408 | MKHDVKEWN |
| 45409 | FSTQQCCVE |
| 45410 | EKKFHLKYQ |
| 45411 | ALGQFATCD |
| 45412 | GGFRFTAQA |
| 45413 | IALSCQSHL |
| 45414 | MSQFGMLAG |
| 45415 | AMKFVNYFY |
| 45416 | SMHLHMKDL |
| 45417 | ISTLLSYCG |
| 45418 | TEMSLPGME |
| 45419 | NAKLGDACG |
| 45420 | AMKKEGEWG |
| 45421 | SAHSTAVYR |
| 45422 | VTYSCVCTH |
| 45423 | GSNWIYEEA |
| 45424 | VSVIFPQQG |
| 45425 | MVIREWCQC |
| 45426 | MVMRQLFYN |
| 45427 | VSYYMYEAP |
| 45428 | CVIRAHQLC |
| 45429 | YGAAKQAMA |
| 45430 | AVTSSLRDL |
| 45431 | TGQVQGFHY |
| 45432 | DKLMFGEWC |
| 45433 | AASLKPWMY |
| 45434 | TRGLYMWSY |
| 45435 | LSSRSEHPN |
| 45436 | NASMYGAQC |
| 45437 | SIFFFAASP |

### Example 21

AAV5 Variants with Liver-Detargeting Tissue Tropism as Identified by Machine Learning

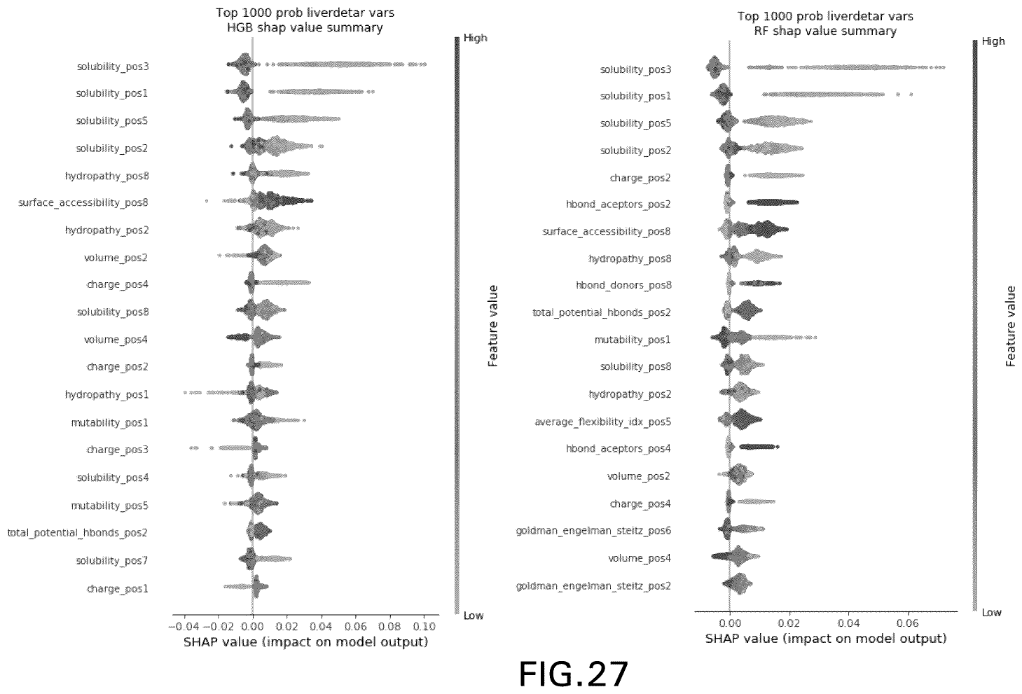

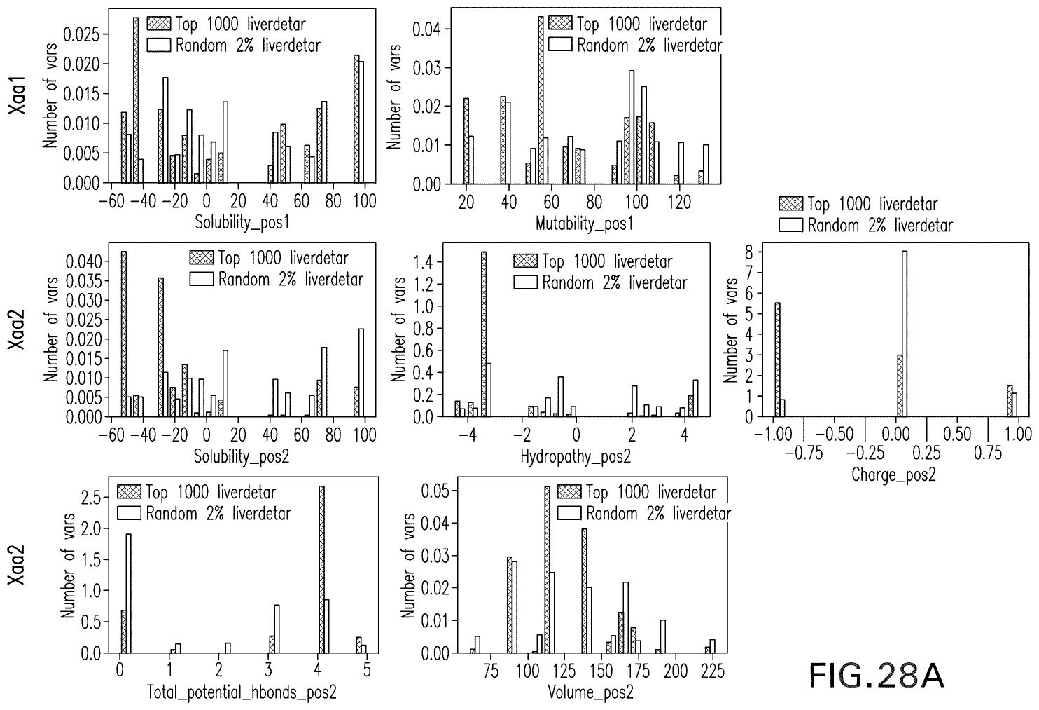

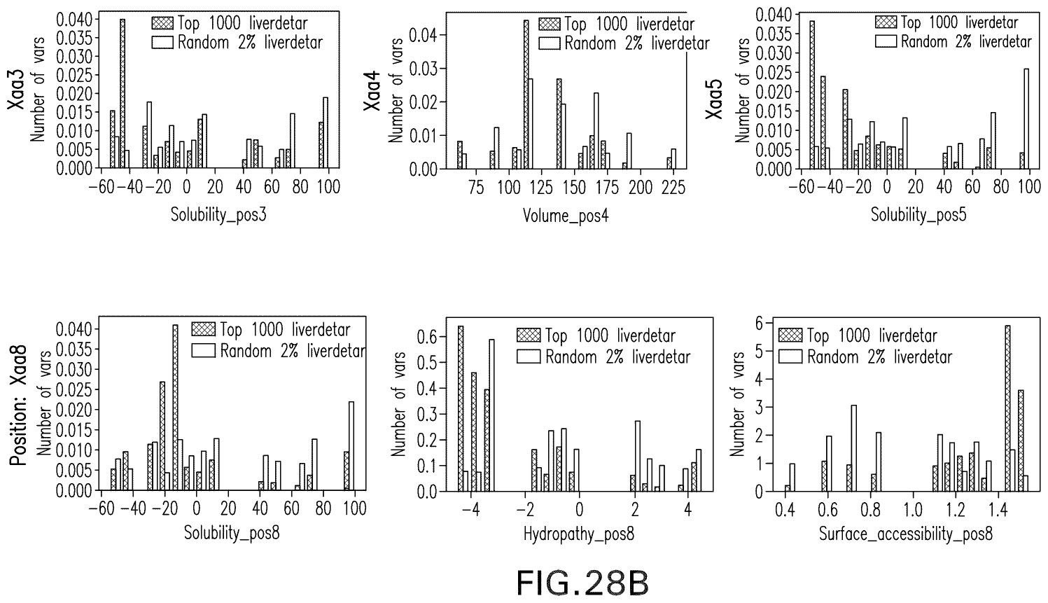

This example describes preferred properties of engineered AAV5 variants that display liver-detargeting tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display liver-detargeting tissue tropism. FIG. 27 shows a set of top 20 positional features contributing to model output probability. Shapley Additive Explanations (SHAP) values can be used to interrogate the relative contribution of features to model predictions. As shown in FIGS. 28A-B, these features were further compared between tissue targeting and non-targeting variants. FIGS. 28A-B show a comparison of top predictive features positionally. Features were selected if they were found to be important to both the HGB & RF models. Summaries are shown of the features in the top 1000 ML-predicted liver-detargeting variants compared to random 2% liver-detargeting variants.

Favored biophysical properties and favored amino acid residues at each position in the 581 to 589 region of an AAV5 VP1 capsid polypeptide, which are associated with a higher probability of liver-detargeting tissue tropism are described below. Any of the below machine learning-derived positional amino acid preferences described in TABLE 42. Listed below in TABLE 42 are a summary of positional features shared between the top 30 important features for liver tropism extracted from two ML models (HGB & RF).

TABLE 42
Machine Learning-Derived Liver-
Detargeting Tissue Tropism Rules

Low solubility at position Xaa1
Xaa1 is selected from D, or P
Low mutability at position Xaa1
Xaa1 is selected from C, K, or L
Low solubility at position Xaa2
Xaa2 is selected from N, K, P, E, or D
Low hydropathy at position Xaa2
Xaa2 is selected from D, E, R, K, H, N, or Q
Low charge at position Xaa2
Xaa2 is selected from D or E
High number of total potential hydrogen bonds at position Xaa2
Xaa2 is selected from H, N, Q, D, E, or R
Medium volume at position Xaa2
Xaa2 is selected from D, E, V, P, N, or T
Low solubility at position Xaa3
Xaa3 is selected from P or D
Medium volume at position Xaa4
Xaa4 is selected from D, E, V, P, N, or T
Low solubility at position Xaa5
Xaa5 is selected from N, P, E, or D
Low solubility at position Xaa8
Xaa8 is selected from K or Q
Low hydropathy at position Xaa8
Xaa8 is selected from K or R
High surface accessibility at position Xaa8
Xaa8 is selected from E, R, or K

TABLE 43 below provides sequences of the 581 to 589 region for variant AAV VP1 capsid polypeptides that exhibited liver-detargeting tissue tropism and comport to one or more of the rules provided in TABLE 42. The present disclosure, thus, provides for rAAVs composed of engineered AAV5 VP1 capsid polypeptides having a SEQ ID NO: 2, wherein the Xaa1 to Xaa9 region is any one of SEQ ID NO: 46438-SEQ ID NO: 47437, as disclosed in TABLE 43. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 43
Sequences of the 581 to 589 Region in AAV5 VP1
Capsid Polypeptide that Drive Liver-
Detargeting Tissue Tropism

| SEQ ID | 581-589 |
|---|---|
| NO | Sequence |
| 46438 | IDGDDEPRA |
| 46439 | PRSDDDQRF |
| 46440 | WDCEDERRH |
| 46441 | DDPDDLECC |
| 46442 | PESDPERTN |
| 46443 | REWWWCLRI |
| 46444 | DDHEEVFKA |
| 46445 | DEDECEGRM |
| 46446 | AEIEKDPRW |
| 46447 | PEPLPEPSK |
| 46448 | YECVERDRF |
| 46449 | PHPQATGPS |
| 46450 | QEHHPRDRW |
| 46451 | CDTEAEFRR |
| 46452 | MEPGEKSRP |
| 46453 | LEMGGKTRT |
| 46454 | CHPHDEHKT |
| 46455 | MDLFPPHRH |
| 46456 | YEDNDVMQI |
| 46457 | PDMKSVVKA |
| 46458 | KWPIEWGHY |
| 46459 | VDRLPPNKS |
| 46460 | MDWDDAIRG |
| 46461 | WDRPPVMRP |
| 46462 | YDWGRVTPN |
| 46463 | REPWQTTEH |
| 46464 | PWKHEPSEF |
| 46465 | FDSHPKIER |
| 46466 | LRDRDGNRM |
| 46467 | RRGMEPTKH |
| 46468 | GDERPGQRN |
| 46469 | PLDRDQATQ |
| 46470 | PKNCRWYRT |
| 46471 | PECGDRGDR |
| 46472 | WDHEPETKH |
| 46473 | EGPDPCNSA |
| 46474 | PECFPKGRN |
| 46475 | CDHDTKHPT |
| 46476 | PADFRDTRV |
| 46477 | CEYDDESQF |
| 46478 | DDEDVSEEI |
| 46479 | HDKPGKPPY |
| 46480 | KNPPWICVW |
| 46481 | PIEKDDNVD |
| 46482 | PKMDKVEPC |
| 46483 | PDPCTEGTC |
| 46484 | ADNCPKNDN |
| 46485 | PDHITKCDE |
| 46486 | DDHDDKLVA |
| 46487 | WEDEPPWKI |
| 46488 | PQPDENYPW |
| 46489 | FDREKRDGN |
| 46490 | GNPHEGQKF |
| 46491 | PEGSMDRRF |
| 46492 | GDWSERSIT |
| 46493 | PERWERPVW |
| 46494 | NDLCQDQRV |
| 46495 | TRPDESPRQ |
| 46496 | WECADRRPT |
| 46497 | YESCNLTRD |
| 46498 | PPPVHNDRS |
| 46499 | VHPEACMRN |
| 46500 | PDQVDRNTP |
| 46501 | REPNSGLVY |
| 46502 | EEPEFTGRG |
| 46503 | TDHNGEIRP |
| 46504 | CDRDPSWRD |
| 46505 | QDRNRVHRQ |
| 46506 | IDPYDCDPT |
| 46507 | PDQDDMMHG |
| 46508 | YEYRPELRA |
| 46509 | CVPEPWDYR |
| 46510 | MDPKHHCES |
| 46511 | QDQCDDKIE |
| 46512 | MHYCDRERK |
| 46513 | PPDRCTAEK |
| 46514 | RNPSQDQKG |
| 46515 | CDTHDVSKD |
| 46516 | DVADVPDTT |
| 46517 | DEKPMDGRG |
| 46518 | PDTSPYCKS |
| 46519 | QLPDKWPKS |
| 46520 | YRCCDDSKT |
| 46521 | PAECISDSQ |
| 46522 | PDPLYWIMN |
| 46523 | LEEGDHMHM |
| 46524 | GDHEPKHEH |
| 46525 | WEVEQRPRH |
| 46526 | CECWPEYKR |
| 46527 | PFPLNPERH |
| 46528 | DVISRNGRC |
| 46529 | PNEDKIKPD |
| 46530 | YPCDDTKPE |
| 46531 | DTDEENSPH |
| 46532 | IDPGMMFRG |
| 46533 | PEQRDRLGF |
| 46534 | DEPSDDDEE |
| 46535 | HHPPKQPPN |
| 46536 | CRWEDYDSR |
| 46537 | CPPQADRIQ |
| 46538 | PQPQFDMMQ |
| 46539 | EFPDGGHKM |
| 46540 | CNCPDQHPD |
| 46541 | WPKCDECRM |
| 46542 | ADNEDARPK |
| 46543 | PICRPDTDV |
| 46544 | APCHDKDRE |
| 46545 | LDPPDSACG |
| 46546 | VETEEKMIL |
| 46547 | MEDTDERSN |
| 46548 | CDRDKRHDH |
| 46549 | GEEADEYHM |
| 46550 | RDIWECIRR |
| 46551 | AECQEDRRG |
| 46552 | TEPDTVDES |
| 46553 | PDPGIRWPM |
| 46554 | PRDVDDSAY |
| 46555 | VEEDQSCKT |
| 46556 | RQYFEDNAG |
| 46557 | YDQWERDIH |
| 46558 | RKPEWSCMV |
| 46559 | LNPQSPEPK |
| 46560 | PPCKMKIDT |
| 46561 | PNDWITDPH |
| 46562 | VERMENPKH |
| 46563 | DAPEPHAQK |
| 46564 | PQEPEEGKT |
| 46565 | HDMDDVLNH |
| 46566 | VDCTPDRRN |
| 46567 | PHQARKNKP |
| 46568 | CEDEGKTWL |
| 46569 | PFPQMNSNA |
| 46570 | ADLRPSVDY |
| 46571 | GEPWDTRIV |
| 46572 | YKPKFDAMG |
| 46573 | PIPQQGGTA |
| 46574 | MDGRDKVDP |
| 46575 | CEDQRQPME |
| 46576 | PDFHHKRDF |
| 46577 | NEGCRDEKS |
| 46578 | LQPNAVGKT |
| 46579 | PKLCSHQTE |
| 46580 | QVPDEGYSW |
| 46581 | NEPDKGYTM |
| 46582 | DLDDVCNKC |
| 46583 | QDTDEGDQC |
| 46584 | FDNPDEYVT |
| 46585 | YPDCEGGKM |
| 46586 | DVNKPDEKW |
| 46587 | EVNDDHAKP |
| 46588 | PHQRNRMDP |
| 46589 | QEPDSNRGV |
| 46590 | SLPQPDTRY |
| 46591 | FEPCPRSCW |
| 46592 | KEPKMEKRG |
| 46593 | PGDHRFYWP |
| 46594 | ADTANILRH |
| 46595 | KPCRDNQDV |
| 46596 | LPPCYCQQY |
| 46597 | PECIEGPNH |
| 46598 | HDNKDVNRN |
| 46599 | GEMTRCFRN |
| 46600 | PDKEKVMPA |
| 46601 | WNSPDDERV |
| 46602 | VDRDDGKDY |
| 46603 | DFEDELSKV |
| 46604 | YEDGDHIVE |
| 46605 | VEGEEILRI |
| 46606 | CEWLDLMRT |
| 46607 | HEAEDWRPF |
| 46608 | KMPCEGEFT |
| 46609 | GDPWEEWRK |
| 46610 | HERWDANPY |
| 46611 | PGCCDLTNI |
| 46612 | PIIKGEPAD |
| 46613 | PDTDDIIHW |
| 46614 | EDLNEKSKS |
| 46615 | EVDDGNVGE |
| 46616 | DDWNEAKRN |
| 46617 | PPDMPMSAD |
| 46618 | QNPWDTDNH |
| 46619 | PDTWAYAPH |
| 46620 | TEPEIRNWF |
| 46621 | FPSDPDGEH |
| 46622 | PPPVVSPKP |
| 46623 | FDPHNPCFR |
| 46624 | ANAEPVHRC |
| 46625 | LVPESQMHG |
| 46626 | WERCPELNQ |
| 46627 | MDEHREQRS |
| 46628 | SDPEKYPWD |
| 46629 | ERDDPRFSN |
| 46630 | FDSSQEWKL |
| 46631 | CRKNPTIHM |
| 46632 | EAEEMGLKH |
| 46633 | CKYLSHERL |
| 46634 | IDPCDELFW |
| 46635 | CKPTSDMKE |
| 46636 | RPWEWWIRK |
| 46637 | FEPQVEANT |
| 46638 | LETAEKSKS |
| 46639 | FDSCSRHRW |
| 46640 | DNTDPDGSW |
| 46641 | ADPQSVCCT |
| 46642 | PHCQTVSDI |
| 46643 | IEEPDTCLV |
| 46644 | PEMQNDMDS |
| 46645 | DHLPEDKRY |
| 46646 | QENDANRPQ |
| 46647 | KQGCEKNPM |
| 46648 | HRCDPDMGR |
| 46649 | DVPRDRKEN |
| 46650 | EDNNNDVKH |
| 46651 | IDFRPPNNT |
| 46652 | DFPDKGELG |
| 46653 | CIPDVAIMM |
| 46654 | MEDHTAPRW |
| 46655 | PENQQNHRD |
| 46656 | CRKSETPWD |
| 46657 | RIPPMMIML |
| 46658 | PQPWNQQAF |
| 46659 | CEIQGGQRY |
| 46660 | VDFHDGFND |
| 46661 | WDDHKLWRW |
| 46662 | RDPFSPDPM |
| 46663 | HLPRDDPVM |
| 46664 | SDIPPNCRK |
| 46665 | DPPRSKDDS |
| 46666 | NDPDTSRTA |
| 46667 | IDWPDNQKV |
| 46668 | YDCENILRT |
| 46669 | VEKPSEEPQ |
| 46670 | LKPLPHFRE |
| 46671 | HDEEEKNKA |
| 46672 | PHGWEENDP |
| 46673 | PLVGMDHRE |
| 46674 | HRPEQLHEG |
| 46675 | PGPHDQGPY |
| 46676 | EVEDRSGWC |
| 46677 | PEPDWVQEC |
| 46678 | CRSEGWIHT |
| 46679 | PETTSRWKW |
| 46680 | RDYQDFICY |
| 46681 | CDFQSSWDQ |
| 46682 | IECFPRESI |
| 46683 | PERASEMFW |
| 46684 | NYPRDHRQH |
| 46685 | FEAHDRPSN |
| 46686 | WEMNDMQDY |
| 46687 | LDTCEMLKQ |
| 46688 | QFPEFDRET |
| 46689 | PVPHPEKEF |
| 46690 | EFDEFNTRG |
| 46691 | GDQAGATRF |
| 46692 | EPPTNDART |
| 46693 | AEVGTEHKV |
| 46694 | DDPSGQLRL |
| 46695 | EDPHSKKEH |
| 46696 | CRKEQGQRT |
| 46697 | PEAGGKSWD |
| 46698 | PWDPEEDGD |
| 46699 | ENPEMMGFE |
| 46700 | MNPNEKGGL |
| 46701 | PDAAERKGV |
| 46702 | EPPDGQVDA |
| 46703 | FDANDCRPS |
| 46704 | FRSHDREEN |
| 46705 | IKCEEPMRL |
| 46706 | FIPDTDNWG |
| 46707 | IDCRSRLEE |
| 46708 | CKEQPTERF |
| 46709 | DDMEDQQQY |
| 46710 | DKPEQLKEH |
| 46711 | PDSHKQPDC |
| 46712 | CDPSVEDRV |
| 46713 | PEGCHHQEE |
| 46714 | FDPCQGGEQ |
| 46715 | NDEDNMSRH |
| 46716 | PECLPKNSC |
| 46717 | AEDASRDLF |
| 46718 | CNPRPQMVG |
| 46719 | CDDHAKRNA |
| 46720 | MRNYPDARF |
| 46721 | DNPPGRRKP |
| 46722 | PDNSDATHQ |
| 46723 | MDCKEWNKQ |
| 46724 | GNQCELNPH |
| 46725 | PNYKKRSTV |
| 46726 | LRCCGMETE |
| 46727 | PPDMQSAKW |
| 46728 | DHPSNDAEM |
| 46729 | RIPGSRPPQ |
| 46730 | WDPNQGGAT |
| 46731 | PSCAPCLEK |
| 46732 | IDSHCKMRE |
| 46733 | CEYDGTMDT |
| 46734 | EVADENGNY |
| 46735 | PQPNIPVRW |
| 46736 | WDYNSIFPG |
| 46737 | IHSNDQPKG |
| 46738 | PHDLKHQRS |
| 46739 | PIPLMMHDE |
| 46740 | FDCLPDREH |
| 46741 | PNDWHIAYG |
| 46742 | LDTKRTFKK |
| 46743 | CDRASDEGK |
| 46744 | DDSSQEKRW |
| 46745 | PEHQDEQSC |
| 46746 | TEPETYPAT |
| 46747 | FKDWDHHTV |
| 46748 | WKTCPQEAY |
| 46749 | EEKDEQERA |
| 46750 | WPNTPWEPH |
| 46751 | CPTGDQQKW |
| 46752 | RPCMQQDPH |
| 46753 | CDAGLISKM |
| 46754 | EASDMSIEK |
| 46755 | AEPRAIWRH |
| 46756 | FHISPKEKW |
| 46757 | PIPPIKTHS |
| 46758 | PDPQNHQFV |
| 46759 | TEVEQLPRA |
| 46760 | IDPNNLTSM |
| 46761 | LGDEPWAKF |
| 46762 | LNHCDKEDH |
| 46763 | WEDHDLHRN |
| 46764 | PALYRRKTD |
| 46765 | NEDIPDPPH |
| 46766 | WEIQDVIRV |
| 46767 | PENWNTGKH |
| 46768 | PDRYPAFEM |
| 46769 | HNPWFSCRW |
| 46770 | INQKETWKF |
| 46771 | PVPYSGPKQ |
| 46772 | YEREPRMFH |
| 46773 | LDEEPVFVG |
| 46774 | PVDADHQLW |
| 46775 | RPPHVGPAL |
| 46776 | ADPTTETPH |
| 46777 | PDWWPRCKP |
| 46778 | YQPRSTPPG |
| 46779 | NNGDDRRQH |
| 46780 | REWKDNCKQ |
| 46781 | PDDWSGTLY |
| 46782 | PEVYPGGTT |
| 46783 | PKCCCKCDW |
| 46784 | PDCRWGEDN |
| 46785 | CDLPMSWRV |
| 46786 | PEDCDDRIS |
| 46787 | QDCEPEYDF |
| 46788 | DVSEDHFND |
| 46789 | PKQLDQPTN |
| 46790 | EFHDMDMVT |
| 46791 | CEHPESKDV |
| 46792 | TEHKDHFET |
| 46793 | PSNEPSVKT |
| 46794 | GEVTGHERN |
| 46795 | PNPQSHLHI |
| 46796 | CPRCDSRPA |
| 46797 | MEDNTVCSV |
| 46798 | QPPRRAMKM |
| 46799 | SLPEEHPFE |
| 46800 | ANPLFKCIS |
| 46801 | DQSEDLDQG |
| 46802 | DDPEYTLCL |
| 46803 | KFPYLPHRN |
| 46804 | PVGNKDSRY |
| 46805 | WDVDHKLRM |
| 46806 | PQHGAETPH |
| 46807 | PHREEDDHC |
| 46808 | CVPNPLGHI |
| 46809 | NDTGPHGRH |
| 46810 | DDTENLREI |
| 46811 | VDPWDPAQP |
| 46812 | CDPWDWEIE |
| 46813 | EQPHDPSST |
| 46814 | PVPCNTVKF |
| 46815 | KMPNELGYY |
| 46816 | KEREERRFW |
| 46817 | DFPEPYYGF |
| 46818 | QDQRRQKRM |
| 46819 | PRHTMLGPS |
| 46820 | DDREKCNWM |
| 46821 | FEQWKDQKV |
| 46822 | LRPCEGEQD |
| 46823 | CPTKPRYPS |
| 46824 | PSESKLMNY |
| 46825 | CDKNWTLQQ |
| 46826 | PSPPPGHNA |
| 46827 | IKPEPNWSS |
| 46828 | PEETRHNDC |
| 46829 | DDVQVEPKW |
| 46830 | PQNGMLHYS |
| 46831 | FDSRHIEKP |
| 46832 | HEKHSLDRY |
| 46833 | ETPDDWNCY |
| 46834 | CDAEAGFRR |
| 46835 | EEFGPTKPM |
| 46836 | DVPQKAHND |
| 46837 | IHPCEGSQT |
| 46838 | VDLQQEDIY |
| 46839 | AFPDNSKTE |
| 46840 | EELASSDKY |
| 46841 | EEYTGECRG |
| 46842 | NIDDKPSMQ |
| 46843 | WPPEPWCCC |
| 46844 | IDPFCAVRI |
| 46845 | DEYCQWIRS |
| 46846 | PQDEWQNRV |
| 46847 | LNPHVSQIW |
| 46848 | MDPYEVTSC |
| 46849 | WDAFEPMRY |
| 46850 | YDFSERKCF |
| 46851 | MDPNHVHAG |
| 46852 | FDCFDRNLC |
| 46853 | YDTDDAQPN |
| 46854 | WDPHKENTM |
| 46855 | RDPNAQGWT |
| 46856 | EWPHIDAGK |
| 46857 | EMDEKLDGK |
| 46858 | CQPPEEGMV |
| 46859 | PPANDVPHQ |
| 46860 | IECSPRETI |
| 46861 | VDIHKEMRV |
| 46862 | PAEHDPNQV |
| 46863 | PKPPPHYEQ |
| 46864 | PGNPENDPN |
| 46865 | DWNLEDDRA |
| 46866 | CEYWHQVRP |
| 46867 | HDPCTQIKP |
| 46868 | CECKPICAT |
| 46869 | PRVDNGNQL |
| 46870 | PHESCECSG |
| 46871 | ADTKRKRRT |
| 46872 | DEPRDWGAQ |
| 46873 | INEDAQEKM |
| 46874 | LNSEPKCRA |
| 46875 | PMPNTGDEE |
| 46876 | TVDEQGIQH |
| 46877 | FDGCSKMPM |
| 46878 | AEPDTIDWD |
| 46879 | DIWDTEVQE |
| 46880 | PQDLKPGNS |
| 46881 | IDRCDVLQH |
| 46882 | HDPWNVLDW |
| 46883 | EDPCSPAAQ |
| 46884 | MNTNENCRK |
| 46885 | CKHVDWDSK |
| 46886 | SEGMDRDRC |
| 46887 | YKELDRDPQ |
| 46888 | PRLQSRKYL |
| 46889 | PGLWIAPDH |
| 46890 | PYLHSKQDK |
| 46891 | PQCHDHRQD |
| 46892 | EQPTQTPFS |
| 46893 | HPPKRHIPC |
| 46894 | VDTQKTERA |
| 46895 | DVDDLVEIM |
| 46896 | CELNGKIDL |
| 46897 | EEENCEQGN |
| 46898 | DEDERTLDI |
| 46899 | YPPSQEIKT |
| 46900 | PHSEEGREI |
| 46901 | EQPECETNV |
| 46902 | PHCNDGSEP |
| 46903 | CECWGCTRG |
| 46904 | PNDFTTHGA |
| 46905 | CDTNPQDQV |
| 46906 | PRWQSEECQ |
| 46907 | VEATPKSPN |
| 46908 | PEEGSPAEY |
| 46909 | DTFQADKRY |
| 46910 | PYEKEKKKD |
| 46911 | DFYHHDDRG |
| 46912 | DTEEPVVNP |
| 46913 | PNEPNEEHK |
| 46914 | VRIYDRCEM |
| 46915 | LRCDPWLPQ |
| 46916 | LEWSEKINP |
| 46917 | VTPEETVVI |
| 46918 | PDPPKQYED |
| 46919 | HPGHDRPDF |
| 46920 | EHGHDDQHR |
| 46921 | DVENQDWAM |
| 46922 | PHSEKYAKH |
| 46923 | PDCLDEGAA |
| 46924 | QDCSDWTKF |
| 46925 | QDHENNLKT |
| 46926 | ADSQDPLQQ |
| 46927 | PLESHESEF |
| 46928 | HDDTHRIVN |
| 46929 | CNEFKHERR |
| 46930 | NEARDGEPF |
| 46931 | PQNPSEWPP |
| 46932 | CRCDTKIRE |
| 46933 | QEPLSKHGV |
| 46934 | INPEIRQIE |
| 46935 | YDLSPKMYY |
| 46936 | EVSDYQQWV |
| 46937 | RPPHKMSKE |
| 46938 | EDRCPGNPG |
| 46939 | LMPDPVQGI |
| 46940 | SIDRDHPKE |
| 46941 | ADPQVEWAM |
| 46942 | CQRQEPDRE |
| 46943 | DAMEDSKTC |
| 46944 | PDPKAWSNY |
| 46945 | PQPKQCMCW |
| 46946 | DMSDEGQKT |
| 46947 | QEPFTESKS |
| 46948 | YECDSEHEV |
| 46949 | PHGTEDWDY |
| 46950 | VRPHDVFYM |
| 46951 | FHPDKLMPM |
| 46952 | QDRRDDHGG |
| 46953 | LKLRDEFNE |
| 46954 | CEVQDEWTG |
| 46955 | EVPNNHHYT |
| 46956 | PEPSWKYRP |
| 46957 | CPWCDTQTY |
| 46958 | KDKCDYMTV |
| 46959 | CATDPIHRV |
| 46960 | CESFPGPVM |
| 46961 | PHVCGEGKI |
| 46962 | LREEELMTR |
| 46963 | WNPANMFNI |
| 46964 | CDPCMECGS |
| 46965 | LHPPPHQCP |
| 46966 | NDFSDEPCM |
| 46967 | IDPPGTESM |
| 46968 | PYPQQSHLE |
| 46969 | VRPFDDGPM |
| 46970 | TEPSKMPIW |
| 46971 | PTMKFKDRD |
| 46972 | CHSGDILDT |
| 46973 | PPKMMDNEF |
| 46974 | PSWGGREEE |
| 46975 | LDQNNGPET |
| 46976 | NETPDAWNV |
| 46977 | WEVPRWPPI |
| 46978 | FDTDQRQSE |
| 46979 | WIPCQFSKI |
| 46980 | PLDHRPHTF |
| 46981 | IWEDMKGKF |
| 46982 | WRIRPQQEI |
| 46983 | PGHFEPTNY |
| 46984 | MEPPDMQCP |
| 46985 | DVPFVNASD |
| 46986 | LRTCPVWRQ |
| 46987 | PIECMGQHW |
| 46988 | VDPSNAAFK |
| 46989 | DPPIDMSKD |
| 46990 | WKPGYGAKP |
| 46991 | CPPSETRVP |
| 46992 | MQDRDTPTI |
| 46993 | RHPEGRRVD |
| 46994 | PFKQGRENP |
| 46995 | LDVTPQPEG |
| 46996 | EVGDMAHQS |
| 46997 | PGHHPLEDH |
| 46998 | HLPVYAPKD |
| 46999 | DVEAMDFGT |
| 47000 | CTPGFWSER |
| 47001 | IDHIGDDRF |
| 47002 | PQSHKLIES |
| 47003 | CNPPCTPFN |
| 47004 | DVCDDHKPT |
| 47005 | PPPLAGWMW |
| 47006 | YDPNMFPYT |
| 47007 | IKPKDQAPL |
| 47008 | ERCHDTEKY |
| 47009 | CKKESTCAL |
| 47010 | PKPVQPHND |
| 47011 | DPELDIPEE |
| 47012 | HEDTHVLRQ |
| 47013 | ESEEHRVMS |
| 47014 | PMEEMHSHE |
| 47015 | DEEVEGMSV |
| 47016 | DEPFFAIRP |
| 47017 | YEMMPNIKY |
| 47018 | EYPENSSHF |
| 47019 | WNPGPVLTH |
| 47020 | GERNGGWRL |
| 47021 | PADIDDDGN |
| 47022 | QRWSDGDKC |
| 47023 | AKICDADQL |
| 47024 | VEPRYDDPC |
| 47025 | PSEHWGCDG |
| 47026 | RNCCGPNPY |
| 47027 | PEQMDRAHG |
| 47028 | MVPDQYQMS |
| 47029 | WVPDLEDVQ |
| 47030 | IDKITWQKH |
| 47031 | DDWTPYLNE |
| 47032 | PDQSEMTKA |
| 47033 | FHPDRRNPT |
| 47034 | QDTNETNDV |
| 47035 | WNEIDEGRW |
| 47036 | DDNQSRGCP |
| 47037 | EIQQHDNVI |
| 47038 | SPPDWWPFD |
| 47039 | MEPDMRQYS |
| 47040 | DIEFLDCET |
| 47041 | LEMQQLTKV |
| 47042 | IEELEEESH |
| 47043 | EDWRKDNNF |
| 47044 | PDMKDFKQE |
| 47045 | WPWKPPYSD |
| 47046 | LQPDRECIG |
| 47047 | WDMMFPCKW |
| 47048 | PHDPGECYP |
| 47049 | PQDHMVNMQ |
| 47050 | DNDEQLGGC |
| 47051 | CKPHQQRIH |
| 47052 | GDMNVWVRY |
| 47053 | CEGAAWPQI |
| 47054 | SVDDFSSHV |
| 47055 | VIPESMNHA |
| 47056 | DDPCMRCNV |
| 47057 | KKPWDDHTR |
| 47058 | SWPQGDYRF |
| 47059 | PVCPMKQRS |
| 47060 | DGEEDWMDP |
| 47061 | MESEESVKP |
| 47062 | QDTESKFRD |
| 47063 | DDMDLIIKH |
| 47064 | ENPSNWHDW |
| 47065 | HHVSMDNKH |
| 47066 | NRDWFEWKM |
| 47067 | CQPESVWHP |
| 47068 | PPCGKGTLC |
| 47069 | PYMLCNSRS |
| 47070 | GEPEAKGNG |
| 47071 | VNLDEMPKA |
| 47072 | ADRCEQIVL |
| 47073 | VNPGQESGE |
| 47074 | CMPDIMTWS |
| 47075 | ENHENCDQD |
| 47076 | MDDMDRRNS |
| 47077 | MDWCEYHRS |
| 47078 | PDHERVEYC |
| 47079 | IRPIICRKG |
| 47080 | PPEHCEHTQ |
| 47081 | LEPPGLDSM |
| 47082 | LKTCPEGNT |
| 47083 | WDKCAWAWD |
| 47084 | MRVCDELEP |
| 47085 | MRHDPFHRR |
| 47086 | LVPDDRLWD |
| 47087 | VWDDKSEAE |
| 47088 | VNPCSIMKA |
| 47089 | GPDEEWNIQ |
| 47090 | PMDEFGHYV |
| 47091 | PGEENEPPA |
| 47092 | ENDEASICM |
| 47093 | PSRTEGDKM |
| 47094 | PDRQQPMQH |
| 47095 | PNQLDKPAM |
| 47096 | EHPNDKIAI |
| 47097 | CNMATSNRS |
| 47098 | PWPDHQRAC |
| 47099 | WPDSRNSPN |
| 47100 | FDDRKRDAR |
| 47101 | LYCEADRKI |
| 47102 | PNENKGQGF |
| 47103 | TYPDVCIGW |
| 47104 | PQQQEGGTE |
| 47105 | PPNFKIYKE |
| 47106 | LPAGDEKGE |
| 47107 | REPLVTEGG |
| 47108 | LCDEGDMPS |
| 47109 | TEDFENHDG |
| 47110 | NEPQSPVGY |
| 47111 | WDGIDRQNT |
| 47112 | MKVTDKLDD |
| 47113 | QDETDEWQR |
| 47114 | QEFIGEERT |
| 47115 | QDDIDLASW |
| 47116 | PTDAALHGN |
| 47117 | PTINDWKTK |
| 47118 | PMDHEKRYH |
| 47119 | YKCDDNFIP |
| 47120 | RDSNCHFDK |
| 47121 | YNDFNRGEW |
| 47122 | PCDQTLIWQ |
| 47123 | DFGCKDEST |
| 47124 | CNLCTHAKC |
| 47125 | ANPDSSVMH |
| 47126 | FRDGDAAEE |
| 47127 | PFPCQQDIF |
| 47128 | VKNAERPRE |
| 47129 | CDFCRQFIK |
| 47130 | PDTSNKTFH |
| 47131 | HERCSELEY |
| 47132 | TDANRLCRK |
| 47133 | PNGCDRSGG |
| 47134 | LESDSFDRD |
| 47135 | PDLQKRQGA |
| 47136 | YEYWPDVSW |
| 47137 | CPWWDFAEC |
| 47138 | DQKDVKGKM |
| 47139 | YELCPLPQH |
| 47140 | DVEDCLICC |
| 47141 | IMPEKMHTD |
| 47142 | PHAEDRDNN |
| 47143 | TVPDVRDCQ |
| 47144 | EQHRDTMRP |
| 47145 | CVMPHNHRL |
| 47146 | PQEDEGVRQ |
| 47147 | SQPNPEINW |
| 47148 | PIDGNSHKD |
| 47149 | PGEPADIKT |
| 47150 | PDHWECIHD |
| 47151 | QQPPLAPDV |
| 47152 | YNPQNHVEG |
| 47153 | GDNAKDFRG |
| 47154 | FNVSWENKP |
| 47155 | PERLNGPTP |
| 47156 | FDPHRCSCH |
| 47157 | DRLRDDYPC |
| 47158 | VSEDDESHQ |
| 47159 | FESTQEPIA |
| 47160 | CKNFPMKNN |
| 47161 | PVPMSHIMQ |
| 47162 | HNGIEKTRE |
| 47163 | KDPCADRWA |
| 47164 | EVVDNMRSQ |
| 47165 | ADYKDWAEH |
| 47166 | PRNLPAYHS |
| 47167 | PMSQICCDM |
| 47168 | YDCWAKEAT |
| 47169 | ENPEVWIEQ |
| 47170 | IERWGVIDV |
| 47171 | DADDRVEAT |
| 47172 | CKDRPIIDC |
| 47173 | NGDDHVFRE |
| 47174 | DYIRIFDRH |
| 47175 | QDHEREEYK |
| 47176 | AEAKERFEM |
| 47177 | PDDEKIEHP |
| 47178 | MNPAVEKGM |
| 47179 | CDPQYDQED |
| 47180 | YEAHSDDRK |
| 47181 | MDPCIEPGR |
| 47182 | CQDEDEAMQ |
| 47183 | PIAQDKVWS |
| 47184 | PRLTDIREV |
| 47185 | VPPFLDIRD |
| 47186 | PEHPDPFSQ |
| 47187 | YWCLDDPRD |
| 47188 | ERVEDGMDH |
| 47189 | RDYAPLLID |
| 47190 | QVPEIQSMP |
| 47191 | PRNGPYCDI |
| 47192 | CDNGEDDHH |
| 47193 | PFDTMDYRS |
| 47194 | PNTHPEYSK |
| 47195 | WNREDKRAQ |
| 47196 | PDWCQDPGM |
| 47197 | PQQPHWCDG |
| 47198 | PPDPPGDNV |
| 47199 | SPPNLRAFP |
| 47200 | GEEDEQSNH |
| 47201 | CHPTHQIRC |
| 47202 | PFPYHLIVC |
| 47203 | PNNQARGQC |
| 47204 | KNPTWVMSD |
| 47205 | YRHEEYWHN |
| 47206 | LEHENGTPQ |
| 47207 | QDPHAHRDD |
| 47208 | VDLCRKQVK |
| 47209 | PYWCDWPNG |
| 47210 | YPCQNHWRT |
| 47211 | MIPCHDRTY |
| 47212 | QPADPPNDS |
| 47213 | PKHADALRM |
| 47214 | LEPCTDEFG |
| 47215 | PDCDPAHHD |
| 47216 | CRPQAIWLH |
| 47217 | HEEIPQQPK |
| 47218 | CEDLNKPMM |
| 47219 | HNPSGPEFS |
| 47220 | GDFIPKQEV |
| 47221 | CKTQERANS |
| 47222 | SDDDREREA |
| 47223 | FEPVSGETP |
| 47224 | PHSDSWCDR |
| 47225 | WHKRDDKTQ |
| 47226 | PYPPMWRGM |
| 47227 | IEDVWECPY |
| 47228 | DYDWSDMKC |
| 47229 | IEMHEKKEE |
| 47230 | CEPRDCNFY |
| 47231 | PTMRESMES |
| 47232 | EVPFKTSTT |
| 47233 | VSPHKTSRN |
| 47234 | ADHCNWSRT |
| 47235 | SDHDNVFED |
| 47236 | SRPGDRGVQ |
| 47237 | QEPDSYACM |
| 47238 | DEECQRVPM |
| 47239 | VDNMSEGRQ |
| 47240 | EQEHDEEKP |
| 47241 | PEFGWWIAM |
| 47242 | PDELMGWQQ |
| 47243 | CECAGWSYE |
| 47244 | MEPFHVNKV |
| 47245 | HKDWHDADL |
| 47246 | PCNCDSWQN |
| 47247 | NEDWEAPEG |
| 47248 | PIEAMHDGW |
| 47249 | MIPQSEERW |
| 47250 | YKTGDEYGL |
| 47251 | PLDKEPMER |
| 47252 | HEDHDPAQE |
| 47253 | GNQDRTNRK |
| 47254 | CQTRDTLEL |
| 47255 | EADEQKTRW |
| 47256 | TEADEEWDT |
| 47257 | GDKWIGCEL |
| 47258 | ECDCRDTPC |
| 47259 | VQPQQMHKT |
| 47260 | PKCCSGMTT |
| 47261 | YNPFYGADM |
| 47262 | CPWCDMKPW |
| 47263 | KDRDTCRAD |
| 47264 | SELTNWVRW |
| 47265 | YNPAACVEL |
| 47266 | CVMPTIYRG |
| 47267 | EIKPYDARN |
| 47268 | EFSGHDEPD |
| 47269 | VDIKPKGAI |
| 47270 | MEQWDPDEW |
| 47271 | VEVTPGFKF |
| 47272 | PPNLCWNQH |
| 47273 | SRVKDCHKA |
| 47274 | ENEKPRTRC |
| 47275 | PHDWSPEFA |
| 47276 | PHQGDREGF |
| 47277 | IESDDIRTQ |
| 47278 | HDDPPGGGF |
| 47279 | EIQDLLCKA |
| 47280 | CVEDHWLHI |
| 47281 | HIAEPKNKP |
| 47282 | HEWKCEKRV |
| 47283 | YEATPHGRQ |
| 47284 | YPPNEHWQC |
| 47285 | LNPAGKIWD |
| 47286 | PEVGSPYPS |
| 47287 | IERHEHNPY |
| 47288 | HDPPKLGCM |
| 47289 | NDDCEESQK |
| 47290 | PVVERFNDP |
| 47291 | PFEEYEQTD |
| 47292 | VRPEKQRVG |
| 47293 | YSDEQGICQ |
| 47294 | HNVPDVRRN |
| 47295 | EADERSKEQ |
| 47296 | PYIWQAMHR |
| 47297 | KDHIAEHRL |
| 47298 | AKPKCPPEG |
| 47299 | DDDIGADSV |
| 47300 | DDPFENIGV |
| 47301 | WDGRTNDKA |
| 47302 | EGDDCMWCR |
| 47303 | VNRRRRDEM |
| 47304 | TEVDDKICY |
| 47305 | EHGDEMHPP |
| 47306 | LEKFPYQPL |
| 47307 | FDEMHTCYD |
| 47308 | KCPDEMNNI |
| 47309 | IDTGDWLVG |
| 47310 | PVPCCHYDP |
| 47311 | PGTEDYSRW |
| 47312 | LNYQPCCRI |
| 47313 | PAEEYHSRE |
| 47314 | PEELYLYRK |
| 47315 | MDVSDQWPE |
| 47316 | WFPDHMSGI |
| 47317 | CPPPSHHID |
| 47318 | LPPMKQDEE |
| 47319 | WRPCAYCQT |
| 47320 | CDDNGCTRD |
| 47321 | RYPEPDAQV |
| 47322 | QVPIMDDFF |
| 47323 | PKLPEYTDS |
| 47324 | MNSEPVTET |
| 47325 | HVPENFVQC |
| 47326 | PDRCHSGDW |
| 47327 | YPPPPNQHH |
| 47328 | IFPYTDKFI |
| 47329 | PGDCYWLER |
| 47330 | HEHRPYFRV |
| 47331 | DLFNHDSPH |
| 47332 | YDDNNWGSA |
| 47333 | HDSGPTFDM |
| 47334 | EIDLEDAQK |
| 47335 | PQEDKQCKP |
| 47336 | DVVHLDWKE |
| 47337 | IVPCLWPIP |
| 47338 | WEPVNAAMA |
| 47339 | QEPLRCIPG |
| 47340 | GNCEPSEPE |
| 47341 | DNVNNKQKG |
| 47342 | PDWFKADDT |
| 47343 | KHPAELDAA |
| 47344 | PTGHQRGTF |
| 47345 | WENEEDKVT |
| 47346 | QNWYDKLPT |
| 47347 | SDARECDIM |
| 47348 | QDSQDQQQW |
| 47349 | DNCDAGKWD |
| 47350 | VKDLADPPG |
| 47351 | PSAPNMWKQ |
| 47352 | ENEETWIFY |
| 47353 | WDNMDQTQG |
| 47354 | EYPCSKMRY |
| 47355 | QCDNESDSM |
| 47356 | SDPDIACVH |
| 47357 | HDDQMHCSS |
| 47358 | CEEETIEQS |
| 47359 | HRAQDPQQF |
| 47360 | HRTDEPTQN |
| 47361 | DVGTGDDRQ |
| 47362 | VDACSHNRS |
| 47363 | FDHQNWFEQ |
| 47364 | VNCPDQHDV |
| 47365 | CDMAHMLPV |
| 47366 | KDDCSMMPQ |
| 47367 | YENKTDMKF |
| 47368 | EYPDNQWSQ |
| 47369 | CDWPIENRA |
| 47370 | VDHVKRCPV |
| 47371 | WRTSDKYER |
| 47372 | PKIQRVTET |
| 47373 | LDDWMMPVS |
| 47374 | HYPRNLVEE |
| 47375 | NEFQEDKSV |
| 47376 | DRGQDNHRI |
| 47377 | PFNDGGIIE |
| 47378 | CYPCLQDKG |
| 47379 | CTPNHCGGK |
| 47380 | SDTEKMHQQ |
| 47381 | GPCCPKYNC |
| 47382 | TLPADKPKG |
| 47383 | FTEEDEIRE |
| 47384 | PDFQKVVSH |
| 47385 | CNPDHMYCQ |
| 47386 | HETCGENVC |
| 47387 | WELAQKHKP |
| 47388 | YYNEPKAKG |
| 47389 | CKTDEFDNH |
| 47390 | KRPEICEWG |
| 47391 | LDPPNSHVN |
| 47392 | PHDNHRHGL |
| 47393 | PIVDWKRQD |
| 47394 | YRDMPGQHN |
| 47395 | YYPMADPSC |
| 47396 | PVNHKDQMT |
| 47397 | EYEWVDEPQ |
| 47398 | PYPGGAVRA |
| 47399 | RKPWVASRV |
| 47400 | NDPWTPISY |
| 47401 | YRFGPFEQE |
| 47402 | RRLPDFDDV |
| 47403 | QPPESMVQL |
| 47404 | DEWTNKRNS |
| 47405 | CRFTDDSSV |
| 47406 | PWNLPWRPG |
| 47407 | YESNTKSDV |
| 47408 | IEQEDQGYH |
| 47409 | EVLDDMKWN |
| 47410 | FNPMVYAHK |
| 47411 | QVDEWGGHE |
| 47412 | PMLQSRGWN |
| 47413 | PYACHHPDF |
| 47414 | PHTNRECDC |
| 47415 | WYDDDLLAN |
| 47416 | PPGCGWYDN |
| 47417 | WQPTHPARF |
| 47418 | EWDDPYGWE |
| 47419 | DQPWFGWQT |
| 47420 | KHPDQWTMT |
| 47421 | YIDRDDSEI |
| 47422 | VDYRTTVKS |
| 47423 | CDDERMDQP |
| 47424 | GVPIIEPRV |
| 47425 | WPKGTKDEA |
| 47426 | DRVHPDHLY |
| 47427 | NEQHNRDQQ |
| 47428 | QEPKETLMI |
| 47429 | KRWMEVDTY |
| 47430 | LNNKDPPPV |
| 47431 | FPHPDSSKH |
| 47432 | PDHSRLEYP |
| 47433 | LEPIQLDMF |
| 47434 | WPPCHITQL |
| 47435 | CPLRDDDPA |
| 47436 | EDENMKSTN |
| 47437 | PTEPDEQYA |

### Example 22

AAV5 Variants with Skeletal or Cardiac Muscle Tissue Tropism as Identified by Machine Learning

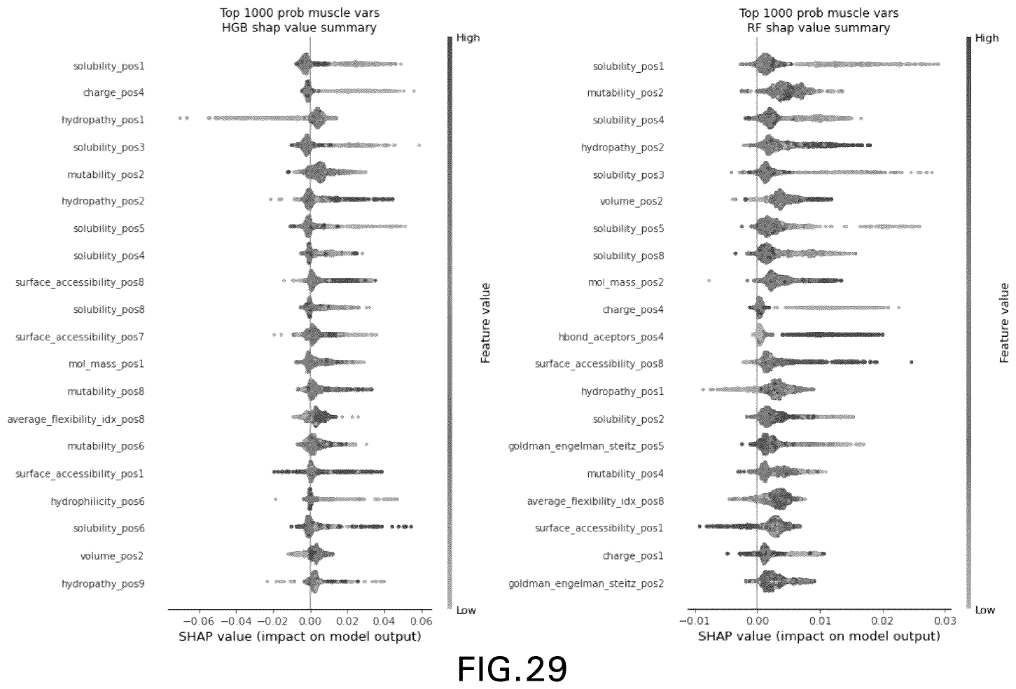

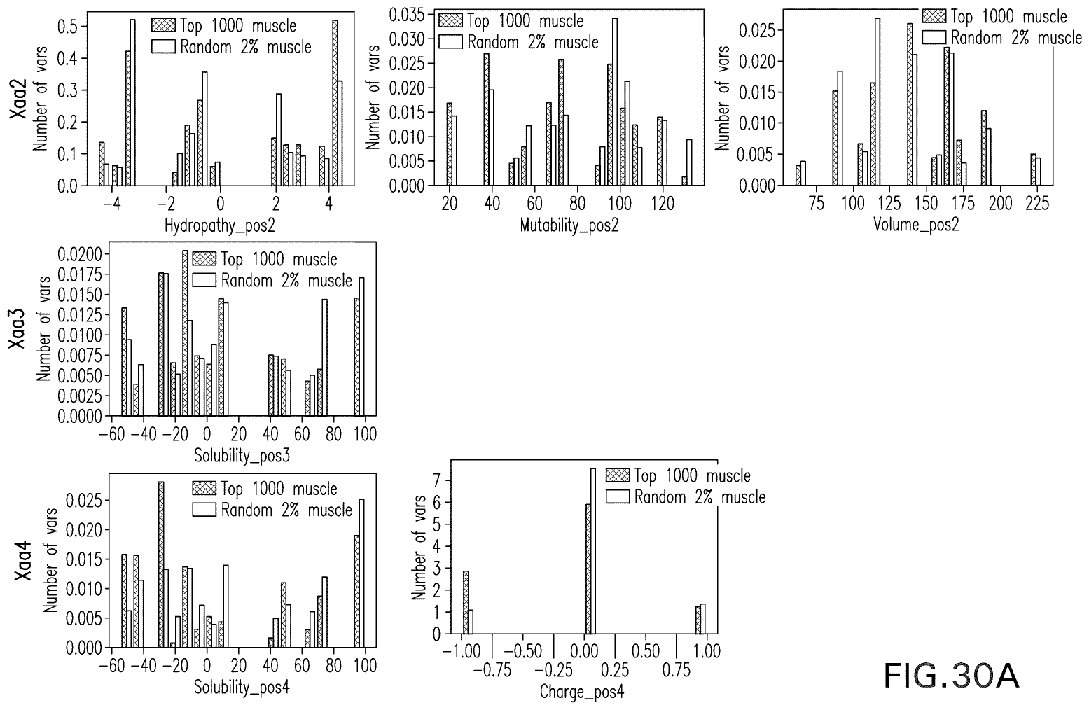

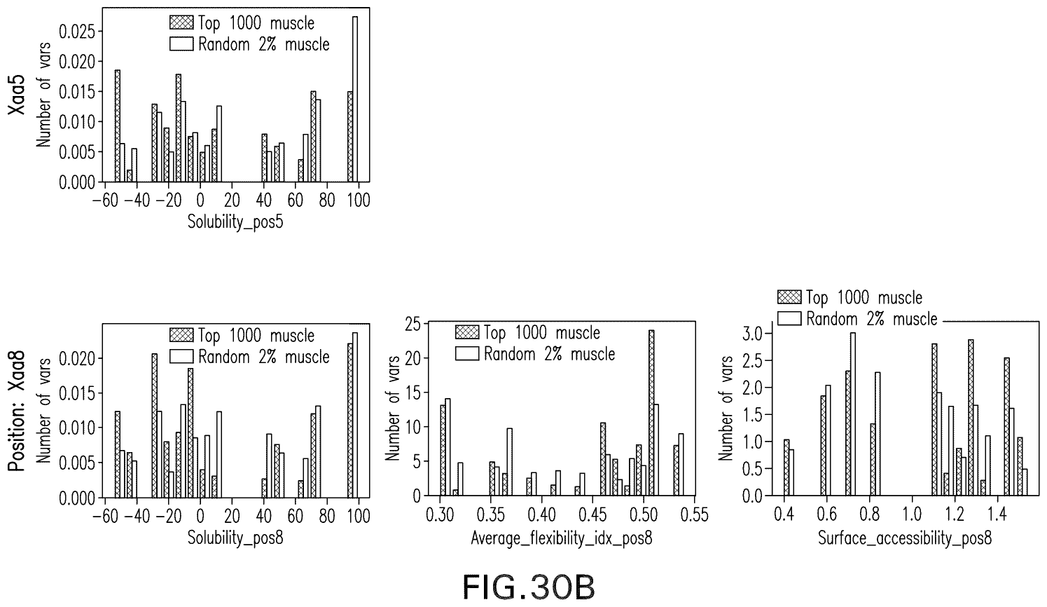

This example describes preferred properties of engineered AAV5 variants that display skeletal muscle tissue tropism or cardiac muscle tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display skeletal muscle tissue tropism or cardiac muscle tissue tropism. FIG. 29 shows a set of top 20 positional features contributing to model output probability. Shapley Additive Explanations (SHAP) values can be used to interrogate the relative contribution of features to model predictions. As shown in FIGS. 30A-B, these features were further compared between tissue targeting and non-targeting variants. FIGS. 30A-B show a comparison of top predictive features positionally. Features were selected if they were found to be important to both the HGB & RF models. Summaries are shown of the features in the top 1000 ML-predicted skeletal muscle or cardiac muscle variants compared to random 2% muscle variants.

Favored biophysical properties and favored amino acid residues at each position in the 581 to 589 region of an AAV5 VP1 capsid polypeptide, which are associated with a higher probability of skeletal muscle tissue tropism or cardiac muscle tissue tropism are described below. Any of the below machine learning-derived positional amino acid preferences described in TABLE 44. Listed below in TABLE 44 are a summary of positional features shared between the top 30 important features for skeletal muscle tissue tropism or cardiac muscle tissue tropism extracted from two ML models (HGB & RF).

TABLE 44
Machine Learning-Derived Skeletal Muscle Tissue
Tropism or Cardiac Muscle Tissue Tropism Rules

Low solubility at position Xaa1
Xaa1 is selected from D, E, R, K, P, N, or Q
Low hydropathy at position Xaa1
Xaa1 is selected from D, E, R, K, Q, N, Y, or P
High surface accessibility at position Xaa1
Xaa1 is selected from E, R, or K
High hydropathy at position Xaa2
Xaa2 is selected from V, I, F, L, or C
Low mutability at position Xaa2
Xaa2 is selected from R, V, I, H, or C
Medium volume at position Xaa2
Xaa2 is selected from E, V, or Q
Low solubility at position Xaa3
Xaa3 is selected from D, R, or Q
Low solubility at position Xaa4
Xaa4 is selected from D, E, P, or N
Low charge at position Xaa4
Xaa4 is selected from D, or E
Low amino acid solubility at position Xaa5
Xaa5 is selected from D, E, R, K, N, or Q
Low solubility at position Xaa8
Xaa8 is selected from D, E, K, P, or N
High flexibility index at position Xaa8
Xaa8 is selected from Q, S, P, E, or D
High surface accessibility at position Xaa8
Xaa8 is selected from S, D, P, N, E, R, or K

TABLE 45 below provides sequences of the 581 to 589 region for variant AAV VP1 capsid polypeptides that exhibited skeletal tissue tropism or muscle tissue tropism and comport to one or more of the rules provided in TABLE 44. The present disclosure, thus, provides for rAAVs composed of engineered AAV5 VP1 capsid polypeptides having a SEQ ID NO: 2, wherein the Xaa1 to Xaa9 region is any one of SEQ ID NO: 25118-SEQ ID NO: 26117, as disclosed in TABLE 45. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 45
Sequences of the 581 to 589 Region in AAV5 VP1
Capsid Polypeptide that Drive Skeletal
Tissue Tropism or Muscle Tissue Tropism

| SEQ ID | 581-589 |
|---|---|
| NO | Sequence |
| 25118 | RDQCRMEAG |
| 25119 | EIREFRASN |
| 25120 | DVCDDHKPT |
| 25121 | EVRIQCEID |
| 25122 | WERCLELNP |
| 25123 | PVMCVWHDN |
| 25124 | CWECCPDSG |
| 25125 | TGCNHNGRF |
| 25126 | PVICVWHDS |
| 25127 | RDQCRMEYG |
| 25128 | KGNPLDGDK |
| 25129 | EINEFRASN |
| 25130 | YSDAKNMSI |
| 25131 | DLLVATEWF |
| 25132 | PYGTIEYDE |
| 25133 | FPKVSECNW |
| 25134 | TQHEDHWLA |
| 25135 | NGVGRPGDN |
| 25136 | AADIFALND |
| 25137 | EDNIQIMMI |
| 25138 | AVCDDHKPN |
| 25139 | TQPEDHWLS |
| 25140 | FPKGSEGNW |
| 25141 | WEQCLELNQ |
| 25142 | TYEALQNSW |
| 25143 | WERCMELNQ |
| 25144 | RYQCRMESG |
| 25145 | SGDWDFGAW |
| 25146 | DLIVATAWF |
| 25147 | VFKPLDNRY |
| 25148 | DSYTDSWST |
| 25149 | PFACDRYEV |
| 25150 | SGDLDFGEW |
| 25151 | KDGRNPDCL |
| 25152 | DLSVATEWF |
| 25153 | SADIFAIND |
| 25154 | CWACCTDSG |
| 25155 | CWECYTDSG |
| 25156 | YKGMEKDPV |
| 25157 | TYESLQTSW |
| 25158 | QTNEEEYGN |
| 25159 | YYNHKIVEQ |
| 25160 | CWEGCTDSG |
| 25161 | AADIFAINA |
| 25162 | PYGPIEYDA |
| 25163 | EAGCKVWPT |
| 25164 | EFGCMYWGY |
| 25165 | DVAPMHQVA |
| 25166 | EVLIQCKID |
| 25167 | NKMIIFDSW |
| 25168 | DVADVPDTT |
| 25169 | HTIMEWSPT |
| 25170 | CGECCTDSG |
| 25171 | DVTTTSWIE |
| 25172 | ECGCMYWCY |
| 25173 | GNEDCNLAF |
| 25174 | HHFNVNLDI |
| 25175 | TQTEDHWLA |
| 25176 | ECGCMYWGC |
| 25177 | YSDPKNMYI |
| 25178 | EIHMFGPSE |
| 25179 | EAMPFRENQ |
| 25180 | YSDPMNMSI |
| 25181 | DLIVATELF |
| 25182 | KEFVQWAAA |
| 25183 | ASDMQFWSM |
| 25184 | DTSDLQVMK |
| 25185 | EVYKNDDGN |
| 25186 | NIDEMNCNA |
| 25187 | YKGMEKTPV |
| 25188 | NTVEKMQTL |
| 25189 | ESYPKLTQF |
| 25190 | RDAWKWKRY |
| 25191 | TIDDKQSWA |
| 25192 | ATMDGHMSM |
| 25193 | WGRCLELNQ |
| 25194 | CQMLCRTPV |
| 25195 | VFKTLDKRY |
| 25196 | EMAELWDTG |
| 25197 | EISSSNLDP |
| 25198 | DLIVATEGF |
| 25199 | FCDPAHHYE |
| 25200 | VMDEYEANP |
| 25201 | ETSEHNRMP |
| 25202 | ECGCMYWGS |
| 25203 | PYGLIEYDE |
| 25204 | EIAGNDTYM |
| 25205 | VDRLFRVEY |
| 25206 | PTEPDEQYA |
| 25207 | SVDQDQPGN |
| 25208 | GWECCTDSG |
| 25209 | SGDWDFGEG |
| 25210 | DMENPNAGP |
| 25211 | VFKPLDKRS |
| 25212 | DVIPRDGAE |
| 25213 | PVICVWHGN |
| 25214 | ITAEVTWQE |
| 25215 | IRTWGCKKQ |
| 25216 | GSHWYDCNP |
| 25217 | RLTEDMSDM |
| 25218 | FDWMANVSV |
| 25219 | THESLQNSW |
| 25220 | FQMLCRPPV |
| 25221 | EIHMVGQSE |
| 25222 | ETADRIHWI |
| 25223 | PYGPIEYYE |
| 25224 | CDHVTKNPT |
| 25225 | EVHTKDEHM |
| 25226 | TVCNHHGRF |
| 25227 | QGDYSWWRY |
| 25228 | GTLDVPTMF |
| 25229 | SVICVWHDN |
| 25230 | HVDITDNDM |
| 25231 | CDHATKHPT |
| 25232 | TYEYLQNSW |
| 25233 | ATSEIQPFV |
| 25234 | EVTEGCPMG |
| 25235 | DNVDLDIQH |
| 25236 | NVLGFDDGG |
| 25237 | SNVMATQNW |
| 25238 | QVTDNAAGM |
| 25239 | AKYLPGLYY |
| 25240 | HNCPWDQQY |
| 25241 | CWECCTYSG |
| 25242 | WNTIMTYES |
| 25243 | PQYVHNWRQ |
| 25244 | HTGMEWSPT |
| 25245 | WDYSMAKKD |
| 25246 | WKHCEREIN |
| 25247 | SIFDYDTFQ |
| 25248 | QTNGEEDGN |
| 25249 | EAALENVNM |
| 25250 | WQKPINCEC |
| 25251 | EIRMFGQSE |
| 25252 | DALEWPMPP |
| 25253 | DIAPDWKGE |
| 25254 | TKMNIFDSW |
| 25255 | MVRPTDQPA |
| 25256 | ETEDQARSC |
| 25257 | IEYWEHETK |
| 25258 | TIVEDAPLA |
| 25259 | YKEMPTILC |
| 25260 | TVLEDSMWA |
| 25261 | AVCDWLIAE |
| 25262 | PSSCDWIPV |
| 25263 | LSTELHHFG |
| 25264 | CTEGENDEH |
| 25265 | PMPDGMFGI |
| 25266 | CTVDGLHFH |
| 25267 | DNYMQLNGV |
| 25268 | DVDGEHEVY |
| 25269 | EVQHELASG |
| 25270 | FTGDFATFA |
| 25271 | TTDQQCTGP |
| 25272 | ESVEKSISN |
| 25273 | AMGEEINYQ |
| 25274 | VAGDIEWYG |
| 25275 | DTQPDTLNP |
| 25276 | AITDDNRMQ |
| 25277 | TNDQQCTGH |
| 25278 | VCEELDREW |
| 25279 | GNGYWMCMS |
| 25280 | HQYVHTWRQ |
| 25281 | SYHMFDYGE |
| 25282 | CMHDEMQTS |
| 25283 | DFNSIDHSG |
| 25284 | DASERNRWQ |
| 25285 | RHNPHDACF |
| 25286 | QYVDQENCN |
| 25287 | DASPQCDRD |
| 25288 | EIKPAAEIT |
| 25289 | DALTRVDGD |
| 25290 | DYQWPDVEE |
| 25291 | DIVENDSLQ |
| 25292 | VIVESEEYM |
| 25293 | GFVDKMVMD |
| 25294 | IVHEWEVNH |
| 25295 | EVMQPEASV |
| 25296 | QTLDKWDGP |
| 25297 | DTFMMQSAH |
| 25298 | DAIPWWWSY |
| 25299 | WERCLVLNQ |
| 25300 | NVEEGMAAD |
| 25301 | EAGPWKRDV |
| 25302 | TIHDVAMMD |
| 25303 | MNLQIGGKG |
| 25304 | NNVRQVGNT |
| 25305 | QFPGGDDEG |
| 25306 | VYVEKMGWY |
| 25307 | TVEHTNHDA |
| 25308 | HQYVHDWRQ |
| 25309 | NLIVATEWF |
| 25310 | MLMHADMTP |
| 25311 | DSSESLNQN |
| 25312 | KCEFYQFCQ |
| 25313 | EVCSQWTQF |
| 25314 | MIDEVWHHW |
| 25315 | PNMPHAEDQ |
| 25316 | TFDDFDMCA |
| 25317 | PWQQHRKMS |
| 25318 | GDPWEEWRK |
| 25319 | EQLEDNDMQ |
| 25320 | MIHSQDMFE |
| 25321 | QVFHQDDVN |
| 25322 | HYKYDANRK |
| 25323 | DMMNIEKMG |
| 25324 | WEHCYRVSW |
| 25325 | DILFWEDDD |
| 25326 | MTDEHLQYT |
| 25327 | LSVDRDYFL |
| 25328 | DVQQIHNKE |
| 25329 | MITNNCNIP |
| 25330 | NISWNESHD |
| 25331 | ETVHPSASN |
| 25332 | TKMIIFDAW |
| 25333 | IIAQWDQVN |
| 25334 | TADDFQGDE |
| 25335 | SQERMAYDG |
| 25336 | EAMQDHTDC |
| 25337 | CDWMANVAV |
| 25338 | DVKPVQKDW |
| 25339 | ANEYQYDTM |
| 25340 | MVVDRMLGW |
| 25341 | MVVEYLTAG |
| 25342 | MPAYSKLKW |
| 25343 | AHEEDEAMQ |
| 25344 | ETRPTVAIQ |
| 25345 | VDNMSEGRQ |
| 25346 | SQESMDYDG |
| 25347 | CVDDLASSF |
| 25348 | EVKPSMRDD |
| 25349 | LCSQPIPEP |
| 25350 | ETRPGMSDS |
| 25351 | ETSFKIHEH |
| 25352 | DTVEGGYIQ |
| 25353 | YYEHPINKK |
| 25354 | SIDWINQQG |
| 25355 | EITPYAKWP |
| 25356 | EMCERNAGI |
| 25357 | DVCTNPWYT |
| 25358 | NDYFRVYRK |
| 25359 | SQEHMDYDG |
| 25360 | LGPWPQTDQ |
| 25361 | AGEFVQANW |
| 25362 | TVKPHDNTE |
| 25363 | QDEMMMACI |
| 25364 | VTWEDVYIN |
| 25365 | NVHESDTMF |
| 25366 | NIAGYEWWM |
| 25367 | WLLGMWNTG |
| 25368 | VINPHDRSV |
| 25369 | TVIPYEMFD |
| 25370 | PSTNLTDSA |
| 25371 | SVYDDFTST |
| 25372 | NIHGVNLQG |
| 25373 | DWEKVPCMK |
| 25374 | TQQVADYTK |
| 25375 | FIGSSDKQS |
| 25376 | CLDELVEED |
| 25377 | ATLDLDSHA |
| 25378 | DTQQEQSQC |
| 25379 | TAEIFAVND |
| 25380 | DVVFNLWAP |
| 25381 | GSMSSTVPH |
| 25382 | NVVMLDQSY |
| 25383 | QQDMIVTKQ |
| 25384 | DFMFLISTH |
| 25385 | FQVDTFMFE |
| 25386 | IFEWGDATY |
| 25387 | LPPGIEWEH |
| 25388 | MDRLAEFKD |
| 25389 | DTVVKNMYH |
| 25390 | EQIPVTWTT |
| 25391 | WHMMSRDGN |
| 25392 | STEEGKGWF |
| 25393 | DVMMALATQ |
| 25394 | SAEPQDANV |
| 25395 | SVTMDEEAH |
| 25396 | AVPTSQAEY |
| 25397 | PCAMHNWCC |
| 25398 | EVYVESQNE |
| 25399 | CIDTYDIDS |
| 25400 | SNMELYCRV |
| 25401 | GTHEHEYGA |
| 25402 | DTLPVQVYQ |
| 25403 | NIFDTNVLT |
| 25404 | VSEDKEAIS |
| 25405 | VMHDIPNFF |
| 25406 | ETMSRHEGM |
| 25407 | ATEIDHWQW |
| 25408 | LSMDVTMAG |
| 25409 | AIHWMDWCQ |
| 25410 | TVHFFSEET |
| 25411 | EMAGPFVTT |
| 25412 | DIHVNNFWN |
| 25413 | EKQMDGINH |
| 25414 | QEDGDFSTF |
| 25415 | EITQTYDHL |
| 25416 | AHSEIMCDQ |
| 25417 | VTKLAEDVY |
| 25418 | PEFMWCVPW |
| 25419 | DTTTDMWVA |
| 25420 | DVFIENQHW |
| 25421 | NVSLQSIHK |
| 25422 | ESGMEQISQ |
| 25423 | PNGCYTDCE |
| 25424 | AVKEFHDCF |
| 25425 | WVADHGDGS |
| 25426 | EIGMTQPCT |
| 25427 | WLYTSNFKD |
| 25428 | DDMEDQQQY |
| 25429 | EATTTSEKD |
| 25430 | QTQDTMNLH |
| 25431 | QVHHNNVDM |
| 25432 | SVVLCNLCQ |
| 25433 | DAPDFMTFC |
| 25434 | FPRIEGQGE |
| 25435 | PQLFKGPVE |
| 25436 | TTEWLDFDA |
| 25437 | FEWVDWGYV |
| 25438 | MRNYPDARF |
| 25439 | EIGSCPEWC |
| 25440 | DNPPGRRKP |
| 25441 | TTTDSPMHF |
| 25442 | SCDESEAYY |
| 25443 | WEHVLDYAK |
| 25444 | DASFVDQDE |
| 25445 | GVNEAEFTI |
| 25446 | MIEVTSHST |
| 25447 | AVQHTEFNL |
| 25448 | DIQGMLEAA |
| 25449 | DVVMTAMDY |
| 25450 | FKDWDHHTV |
| 25451 | NVEHNNMGA |
| 25452 | HVHEIMEEE |
| 25453 | SVMTNDDID |
| 25454 | GPIEFGWQI |
| 25455 | EFANTEHTS |
| 25456 | VTEDMIMNT |
| 25457 | SQISVEFNC |
| 25458 | EFWSMWVSD |
| 25459 | DADELRSAK |
| 25460 | EMRTYNKDY |
| 25461 | SSTELFQAG |
| 25462 | TEPETYPAT |
| 25463 | VMDDESHRG |
| 25464 | DACEEGSSM |
| 25465 | WYTQDIEKE |
| 25466 | IGDHDCQEQ |
| 25467 | DVGNTYNHW |
| 25468 | QSYTTMYQC |
| 25469 | ISGEIDYME |
| 25470 | ACCEFELTQ |
| 25471 | TANEWETME |
| 25472 | AEKMDEIWK |
| 25473 | VTKPKWSNF |
| 25474 | VVVLSDFGE |
| 25475 | GSHWYDYNQ |
| 25476 | GMYESNQGS |
| 25477 | DVDCVWHEE |
| 25478 | LVYDPHFTS |
| 25479 | NDVDLWHVD |
| 25480 | AVEYAGWEK |
| 25481 | FYQADIGWA |
| 25482 | NTEAEDHQD |
| 25483 | RCDCKVHEF |
| 25484 | HLPCGWPAV |
| 25485 | IPVHFDWFF |
| 25486 | NVEGIVTAC |
| 25487 | DVGNMAAIE |
| 25488 | ETLQAQHCD |
| 25489 | NTADIHMKE |
| 25490 | SPLEVWTPA |
| 25491 | KFVWDTWAW |
| 25492 | KCFLCGSTT |
| 25493 | WECADRRPT |
| 25494 | VIDDYFQMC |
| 25495 | GATCTIVRY |
| 25496 | TIDCYAAMD |
| 25497 | PYCQQAANW |
| 25498 | MVYEATELD |
| 25499 | MTVDLYEVL |
| 25500 | EVDKGLAQA |
| 25501 | DSELLPEAH |
| 25502 | AVYDHAPQN |
| 25503 | WHHTHSHNC |
| 25504 | DIMPQWAFM |
| 25505 | DIRHYNISR |
| 25506 | VTLDGNRMG |
| 25507 | IIETQCQAI |
| 25508 | EASHIFTGE |
| 25509 | QIAEACAQH |
| 25510 | ETTCNMGAK |
| 25511 | VFDMTNLRV |
| 25512 | LSPWHQTDQ |
| 25513 | ECSHQDRSN |
| 25514 | TMMDAEVGL |
| 25515 | YLTELIQKT |
| 25516 | EIVELRQTR |
| 25517 | FPMVWFVLF |
| 25518 | SNEWPTMIH |
| 25519 | QISEHAGRS |
| 25520 | VVQEKSINW |
| 25521 | DAQPQRNGS |
| 25522 | EVWTSCMTP |
| 25523 | EATNFTIGR |
| 25524 | VTEPHSFTV |
| 25525 | LHEVQTILH |
| 25526 | EVVTQMEHL |
| 25527 | WRVWQNLPQ |
| 25528 | TSEYMATGE |
| 25529 | KDWRFEDEK |
| 25530 | IQAEQMSGC |
| 25531 | DAKNWLDKN |
| 25532 | MPNFAWEAQ |
| 25533 | DLLNYGMFH |
| 25534 | EQPFDEFKA |
| 25535 | HATGLVNVM |
| 25536 | KEEFNQEPA |
| 25537 | EAYTRVGYE |
| 25538 | MQEEMGPRK |
| 25539 | DFTNQDHDN |
| 25540 | EVRANCESY |
| 25541 | DNTDPDGSW |
| 25542 | TIGPSNFST |
| 25543 | DNANFSGAD |
| 25544 | EYMERDMSI |
| 25545 | FDKLGTMWG |
| 25546 | CVDQLDDVT |
| 25547 | ESGGYMRMC |
| 25548 | VQTMPKPIY |
| 25549 | TYVDNNHMV |
| 25550 | EIVVEFEDQ |
| 25551 | IPDCMNLSQ |
| 25552 | HTVMEGSPT |
| 25553 | PIIPIAEHV |
| 25554 | DVQGSGQWV |
| 25555 | HTNQQANSP |
| 25556 | STDDEYSLA |
| 25557 | TAVEWHYFD |
| 25558 | MAPQSLWNM |
| 25559 | SSGEMDHVY |
| 25560 | TIDDYGPIS |
| 25561 | DQPCKMWAR |
| 25562 | TTEAGTALE |
| 25563 | ETDLKCHSY |
| 25564 | ACVEQEKAD |
| 25565 | SMNEMYQYP |
| 25566 | GIDTTTQAF |
| 25567 | FPQMNEWEC |
| 25568 | DVHTTCYHY |
| 25569 | SQPNPEINW |
| 25570 | EVMLSKMEY |
| 25571 | IVDHCNMYP |
| 25572 | EFHQFGNHM |
| 25573 | TVQMGEWGY |
| 25574 | NMSDGHEVC |
| 25575 | DDNEYHVWS |
| 25576 | DNWMGQDDK |
| 25577 | DEMDAQMGA |
| 25578 | TNDWMRLRV |
| 25579 | IHYDYNTIC |
| 25580 | DTGSFTSTP |
| 25581 | LTEATPKWT |
| 25582 | GDWSERSIT |
| 25583 | TTCPMEKLN |
| 25584 | LCVEDNIQQ |
| 25585 | DVAQYGLGN |
| 25586 | TADMMPMNP |
| 25587 | TDESEFESS |
| 25588 | HDMSYHDPI |
| 25589 | TVVERHEHG |
| 25590 | CVETQWRAD |
| 25591 | SFDIESARQ |
| 25592 | EVEVVKEFQ |
| 25593 | NTQTTDIHG |
| 25594 | PQKGDDWLY |
| 25595 | PCEQMPYIM |
| 25596 | DEYDNVWQA |
| 25597 | DMRDSHAWP |
| 25598 | YTCHEEWPL |
| 25599 | ESGLAPMNL |
| 25600 | PMDHEKRYH |
| 25601 | VPEPDDKPY |
| 25602 | AIIWTTDQP |
| 25603 | QSMPICEGH |
| 25604 | DAGPLSMRS |
| 25605 | VTEVQHMAK |
| 25606 | WEGFYMLTL |
| 25607 | MIMCMDCFP |
| 25608 | DIAGMPHIS |
| 25609 | GVDAGLVSE |
| 25610 | TIDVPMQYA |
| 25611 | MELEGHNLP |
| 25612 | DEWYRSKED |
| 25613 | VWDCPKWRH |
| 25614 | SYDDTVICW |
| 25615 | IVDNKCYYN |
| 25616 | DSTPIMQRC |
| 25617 | YWDCQHSYM |
| 25618 | TKMIIFGSW |
| 25619 | SVVIRQLVD |
| 25620 | EVCIHEQCM |
| 25621 | DCSHEAHRY |
| 25622 | INPELSEGC |
| 25623 | DTSSWGVIP |
| 25624 | HYRDNKSLP |
| 25625 | MNSEPVTET |
| 25626 | QMEWLAAGP |
| 25627 | IFTTADGEF |
| 25628 | MFPSEAKEI |
| 25629 | DVKPGLFYT |
| 25630 | ETDQPQLAA |
| 25631 | DVLLYMSAT |
| 25632 | DIIFKLEAG |
| 25633 | CDAGLISKM |
| 25634 | MFTGPDVRH |
| 25635 | GVMESQYIS |
| 25636 | DCTLQPAMY |
| 25637 | PEAQRVAGI |
| 25638 | DVEMCQKMS |
| 25639 | WHGCYKIMF |
| 25640 | VHMPYTEET |
| 25641 | ENGTHQYND |
| 25642 | QCAYSWWRY |
| 25643 | CEWLGTCNH |
| 25644 | DTCYNNWKD |
| 25645 | LSAQRLVGD |
| 25646 | NLDMYSISQ |
| 25647 | SILFYSETH |
| 25648 | MDYDGMAVY |
| 25649 | LCSDEVCWT |
| 25650 | TIEGKSLYC |
| 25651 | ETSMPMEHT |
| 25652 | VEINAKWGN |
| 25653 | IEPWDCQIP |
| 25654 | EEPEFTGRG |
| 25655 | ENDCRIRMA |
| 25656 | CVMHVQCDH |
| 25657 | AIATGWQDM |
| 25658 | EYHGYLEHT |
| 25659 | IQWVHGCLF |
| 25660 | NDCSDCQTL |
| 25661 | AGMEIIVQC |
| 25662 | DHEMPESTF |
| 25663 | VKCFPCQLE |
| 25664 | NEPELKLQP |
| 25665 | DTCQAMHGD |
| 25666 | EWHFVNIGT |
| 25667 | MVQQYNGSE |
| 25668 | DIERFSTVC |
| 25669 | NIAAHSPGP |
| 25670 | HNPCWPFMI |
| 25671 | IVDMPVRHP |
| 25672 | SADEDKCGW |
| 25673 | SFPEAWPIA |
| 25674 | MIVSQQWVW |
| 25675 | HIEYWDQRD |
| 25676 | SLMYEDDYP |
| 25677 | DAAIYVMLD |
| 25678 | MQKNPPKRV |
| 25679 | TMVDWELGK |
| 25680 | QTGLNEYAP |
| 25681 | DDMWCGGLY |
| 25682 | VIYFVGLIA |
| 25683 | HHACGTREH |
| 25684 | DISSNIYDI |
| 25685 | AIECYKMGS |
| 25686 | EESNRGYQH |
| 25687 | MTEVVVSSM |
| 25688 | HEGHPHLSC |
| 25689 | ESCTGEWWA |
| 25690 | MTDTVQEYA |
| 25691 | PRNGPYCDI |
| 25692 | NKMTEARKH |
| 25693 | DNGQVEETA |
| 25694 | DAYVQQPSF |
| 25695 | FVYEWSNLV |
| 25696 | EVRGQGSHY |
| 25697 | WFDCPPCCA |
| 25698 | WREFSEWTF |
| 25699 | QLFAPDMDW |
| 25700 | TYNDFYTAF |
| 25701 | EVGSMPQDW |
| 25702 | SKELGSVET |
| 25703 | DVTISMQAG |
| 25704 | KYNFNLNKY |
| 25705 | VSDWPQIKA |
| 25706 | DTKSRQWDC |
| 25707 | AYEGECIHY |
| 25708 | MAEFEQWLE |
| 25709 | CELHELFDG |
| 25710 | TAEYIDISY |
| 25711 | EIAQFSGVC |
| 25712 | DWNEEMAHL |
| 25713 | WQEYRMTDQ |
| 25714 | SNENICPTC |
| 25715 | LPVDVLVKT |
| 25716 | SADWRHKSP |
| 25717 | EDGQSMCVS |
| 25718 | MHHEYAMAG |
| 25719 | DDHDINGGP |
| 25720 | MTEDQTWWQ |
| 25721 | WNEPWYVRP |
| 25722 | ECQQDSWHY |
| 25723 | EVQRQMSNK |
| 25724 | DMHSFKWWS |
| 25725 | DHGEIPAFT |
| 25726 | EYDKMMYAE |
| 25727 | DADREDFWS |
| 25728 | QQEAEHKGV |
| 25729 | AHEVTFVRP |
| 25730 | QPIDVSAAI |
| 25731 | DIYLKCFIK |
| 25732 | EIFLENRFP |
| 25733 | FTKTNDHNT |
| 25734 | WQALWCLVH |
| 25735 | AFKALDVQC |
| 25736 | DIHSTNWLA |
| 25737 | NLTDKCTAD |
| 25738 | NSEWWSSVL |
| 25739 | TNEIMWCSP |
| 25740 | MVAPSQALQ |
| 25741 | APGEYMDVE |
| 25742 | ECAKTWENF |
| 25743 | KDLHDAIHN |
| 25744 | LAPCSKIYC |
| 25745 | FMWPPQIVV |
| 25746 | HMQEPSIAS |
| 25747 | MEPVMHYLN |
| 25748 | YCKCPTCNT |
| 25749 | ADGPVLQMF |
| 25750 | EVSSCDDFT |
| 25751 | CNEEMEFSC |
| 25752 | MLTESQQSM |
| 25753 | WPGFEKLPS |
| 25754 | SPYCDDRGN |
| 25755 | QYCLTEASH |
| 25756 | TSEWLWVDS |
| 25757 | DNMVWRGAH |
| 25758 | CDNDYLTAT |
| 25759 | VIEYNSNIY |
| 25760 | HVSAIEKAQ |
| 25761 | QVATFECKQ |
| 25762 | LHIHRDVTI |
| 25763 | NHEETEFGY |
| 25764 | MDSCYDYQS |
| 25765 | EVLGCNYWP |
| 25766 | STEDWATLH |
| 25767 | CQTDKNATE |
| 25768 | ALEQEMVKF |
| 25769 | QVTFASASK |
| 25770 | DDEVYVWGQ |
| 25771 | QVMPRSIER |
| 25772 | AVHTFQMWD |
| 25773 | LVGNADHGQ |
| 25774 | NVGNADHGQ |
| 25775 | EDYSGTKWG |
| 25776 | TVPLKTYGG |
| 25777 | TYMPMDCKW |
| 25778 | FIVQQSIWK |
| 25779 | NEQHYEYWK |
| 25780 | LDVTPQPEG |
| 25781 | ETSLPHWQG |
| 25782 | DSKPVMMAN |
| 25783 | VYRHFDMTP |
| 25784 | DSLAAWSKD |
| 25785 | STIPTPIWL |
| 25786 | VQVQDWLPE |
| 25787 | DWNCAALKP |
| 25788 | EVGPSWEQR |
| 25789 | MHDFLTSAQ |
| 25790 | NLPCSQQAM |
| 25791 | DQRCNKAPG |
| 25792 | PLAFSPVQR |
| 25793 | ETDLAMKAE |
| 25794 | NVEWRQACN |
| 25795 | ETTTCWQYF |
| 25796 | AMPYEEVPA |
| 25797 | ESAQFVAYS |
| 25798 | VLDCAMCNP |
| 25799 | AEPHFQNKS |
| 25800 | MNESFPTWN |
| 25801 | EVQCDQIHP |
| 25802 | MDQCFSYNQ |
| 25803 | EVGSQRTAY |
| 25804 | DHSPVKSAY |
| 25705 | YICQQDLIM |
| 25806 | KIDHEQDCY |
| 25807 | LPVEPIDCE |
| 25808 | GLMVGQWPH |
| 25809 | AHIWDTLIP |
| 25810 | ATIVDGSGN |
| 25811 | TANDYGPTE |
| 25812 | NVELPKIHE |
| 25813 | MLPDHWVIT |
| 25814 | HPFNEAVKH |
| 25815 | IISSTTSTG |
| 25816 | IKEHECRRI |
| 25817 | AVHQINVAA |
| 25818 | LVMEALPMA |
| 25819 | GQIWAAMRR |
| 25820 | MWVCKLFVC |
| 25821 | PIHHGEHVY |
| 25822 | NHQCNGLAQ |
| 25823 | TCEEPYNFA |
| 25824 | LVHEKWEQF |
| 25825 | ALAKFRCWS |
| 25826 | ITSVFDNKA |
| 25827 | DVITHWIGQ |
| 25828 | QQASSSGGF |
| 25829 | EGDDLDRHY |
| 25830 | MPELKFSYC |
| 25831 | HQTHQHNIW |
| 25832 | AFGTLEHEP |
| 25833 | TKPTHDVGS |
| 25834 | QVMQNESER |
| 25835 | SDESNWFGQ |
| 25836 | FSADVNNYD |
| 25837 | LWCCTDTEC |
| 25838 | SMEEWGCES |
| 25839 | MHFCIWMRM |
| 25840 | AHNESDDCG |
| 25841 | SHQFVQPNN |
| 25842 | AIHMFGQSE |
| 25843 | PRKCSNEEV |
| 25844 | HFPLGWLWL |
| 25845 | QQLELDGSL |
| 25846 | INHGWEKVA |
| 25847 | HQHCAEFCH |
| 25848 | DCSHNGMRE |
| 25849 | WINQQTLQC |
| 25850 | QERMFEMTA |
| 25851 | QINMSMHES |
| 25852 | MVTARCWLS |
| 25853 | PLWFEPALN |
| 25854 | EIGYRKVYN |
| 25855 | ESVMEKSMF |
| 25856 | NVNHFGMGD |
| 25857 | SVNVDNAEM |
| 25858 | NEQFQCDVH |
| 25859 | NPNCTKQQY |
| 25860 | QMGPHERHA |
| 25861 | ETRMHMDAL |
| 25862 | LNMYEASLS |
| 25863 | MKVTDKLDD |
| 25864 | ADETCAKKF |
| 25865 | INQWLTCRW |
| 25866 | HNVGDMFCN |
| 25867 | SNQPQGQYC |
| 25868 | LEKVINVLH |
| 25869 | SFVHMFQGP |
| 25870 | DASYYSPVP |
| 25871 | VNDDIYQQG |
| 25872 | LEMMGWPIG |
| 25873 | MEHALCTRT |
| 25874 | ECSCVKRGY |
| 25875 | ESGFPNPYH |
| 25876 | STDWSPSLG |
| 25877 | ETDYIQMGA |
| 25878 | DIVTVIKRT |
| 25879 | LAWDNQQFN |
| 25880 | KTKHRNKFP |
| 25881 | MSNDRCSMG |
| 25882 | FRQSVPHWA |
| 25883 | QVGQEAKNC |
| 25884 | YLEWDSWRC |
| 25885 | MSDFPISTP |
| 25886 | MQNLSSVTE |
| 25887 | STEYRNALN |
| 25888 | VIVGNNVGI |
| 25889 | GDPCDQMTN |
| 25890 | MDEYFCGFT |
| 25891 | EPAQSQHGF |
| 25892 | SFDYVMWGC |
| 25893 | DVFKEHYHQ |
| 25894 | HFVQPACEH |
| 25895 | DVANLKHHV |
| 25896 | VSEQIWNAD |
| 25897 | IVPCLWPIP |
| 25898 | GHQEFKGSE |
| 25899 | ANCSSEIYV |
| 25900 | DSTHDIAHS |
| 25901 | TTDRQCTGH |
| 25902 | VISAMNVYP |
| 25903 | AHQFIQWLH |
| 25904 | ETYRYGSQR |
| 25905 | KGNPLAGDT |
| 25906 | NTMQMPMIG |
| 25907 | MYEVLNNDQ |
| 25908 | ICLHDHFRN |
| 25909 | EMKGTLYAY |
| 25910 | APDFRMITR |
| 25911 | PQWMPVNQI |
| 25912 | EFKTQQFDP |
| 25913 | VNEWLDSIY |
| 25914 | GSIQSVMWE |
| 25915 | DFMCDQFVC |
| 25916 | PCSDSTELD |
| 25917 | DQECWQSSQ |
| 25918 | EEPLPSDQK |
| 25919 | QTMKLEHLK |
| 25920 | PGTHWWWDC |
| 25921 | MTVSWDSWK |
| 25922 | DAWKPHQFY |
| 25923 | THEEYQLGS |
| 25924 | TVIQTECNT |
| 25925 | SNTFIDWQA |
| 25926 | LYDWSCAQS |
| 25927 | WREPNCHGL |
| 25928 | TMAEMCGSP |
| 25929 | SETDWQRKC |
| 25930 | QVIIGYCES |
| 25931 | GFDECMFWY |
| 25932 | PCTQIGEIY |
| 25933 | FMDFFGMAS |
| 25934 | DQMLDNTAY |
| 25935 | SQDCQEVGG |
| 25936 | SDHMSERVD |
| 25937 | DVLQREEIC |
| 25938 | VIALYSLAY |
| 25939 | EWNYGGLKC |
| 25940 | EVQYGKYWA |
| 25941 | DMTMYSNAV |
| 25942 | VTNCHTFNN |
| 25943 | SIPCNHCFE |
| 25944 | MTQGYPSLM |
| 25945 | DIASVRQTE |
| 25946 | IVDSASVCQ |
| 25947 | AIEAKMTQV |
| 25948 | ALAKFQGWS |
| 25949 | PAFLGACGF |
| 25950 | ESEEHRVMS |
| 25951 | CQGCVPFKG |
| 25952 | FIEFDAVTW |
| 25953 | TTCSSNREW |
| 25954 | VCNELQKYF |
| 25955 | VPCYHKGRW |
| 25956 | DVKCEPDRG |
| 25957 | LICWYQQGN |
| 25958 | EMQTHYAAR |
| 25959 | AETDHYIPT |
| 25960 | PQSDDWAMP |
| 25961 | NHQWTQRRY |
| 25962 | FEGNYTATK |
| 25963 | CNCPDQHPD |
| 25964 | IFQCLYTGT |
| 25965 | VETYQINDT |
| 25966 | FFEWLLDTG |
| 25967 | SDALGPEWW |
| 25968 | MTNCKLHEQ |
| 25969 | IYEACFWWL |
| 25970 | WCMQEGTMS |
| 25971 | EIRLEHDGG |
| 25972 | HVELSLWNQ |
| 25973 | CWMEFMAGN |
| 25974 | AWDYWNSQW |
| 25975 | NNITTPDAY |
| 25976 | SVTMHSMEL |
| 25977 | TYDHIKFAP |
| 25978 | LQQMENASP |
| 25979 | CTDDKMCPV |
| 25980 | IDSCNTSLN |
| 25981 | CKEMRMDQW |
| 25982 | EPEHGAMDK |
| 25983 | MVLPYQITN |
| 25984 | ETHLINPGI |
| 25985 | CDHDTKHPT |
| 25986 | CSAEIIDTC |
| 25987 | EVGFYMVPR |
| 25988 | AIWSIEHES |
| 25989 | MVNLGYAEC |
| 25990 | ELEGMSRRE |
| 25991 | VDATTGDYQ |
| 25992 | TVGFGLKAS |
| 25993 | WMEASITGP |
| 25994 | WTDGWHTMC |
| 25995 | SVTLGMKPN |
| 25996 | GHVQESWNC |
| 25997 | STVAADWDH |
| 25998 | ESEIAISWN |
| 25999 | NETLDSKHF |
| 26000 | TFMWLDRVG |
| 26001 | TTDSWMDVE |
| 26002 | DEAEEHGCT |
| 26003 | FTPPKLHYK |
| 26004 | QYLTAEFTS |
| 26005 | MSTNNCNIS |
| 26006 | EAWSCGIYD |
| 26007 | NYDTLLETH |
| 26008 | NVEYVNWHW |
| 26009 | FVMPISAQG |
| 26010 | SQESHMTWH |
| 26011 | GIVALDNAD |
| 26012 | TVMWKDYID |
| 26013 | PWCEDPGCS |
| 26014 | TVIYVTDDA |
| 26015 | TQDCSTMPS |
| 26016 | SMCWLENGN |
| 26017 | MVDLETTYV |
| 26018 | DNTQKQNDP |
| 26019 | NVDRCDSWQ |
| 26020 | TYEPCSTSH |
| 26021 | DFHCQTERQ |
| 26022 | TTAPMEMSL |
| 26023 | NAVEGQSHK |
| 26024 | DNYSIKVGQ |
| 26025 | FMMFHMGLD |
| 26026 | ETRFDDRRA |
| 26027 | NTGWQEAER |
| 26028 | WVFMSWCDV |
| 26029 | PCLITVAPH |
| 26030 | DIAVIGAGA |
| 26031 | EATLITYCS |
| 26032 | TTEDMQCDM |
| 26033 | ITMPRVRPT |
| 26034 | LFVMPAAIE |
| 26035 | ALEHTLFGT |
| 26036 | MGCDVMKAS |
| 26037 | DSWPTLMHD |
| 26038 | CYHEECDGN |
| 26039 | ITTPQTQSH |
| 26040 | IHDWYEVGS |
| 26041 | SMQMTSHYH |
| 26042 | TVAVFSIAM |
| 26043 | QGDEINEQA |
| 26044 | PCSQYYPSC |
| 26045 | PFAAVSMVF |
| 26046 | MQLTNMEIE |
| 26047 | VTESIDYVH |
| 26048 | TIAPYTYMD |
| 26049 | DIQLTHIHG |
| 26050 | MYMTVQQNV |
| 26051 | PFHERGLMQ |
| 26052 | TYVNQGMGP |
| 26053 | FVCELQSAN |
| 26054 | ETKSHPVMV |
| 26055 | ENDEASICM |
| 26056 | VVHMFNDQM |
| 26057 | NTFILETFD |
| 26058 | HDFNWVTKE |
| 26059 | WFVTQGAYM |
| 26060 | FWATSHWGY |
| 26061 | IPYTEKADI |
| 26062 | HDEEEKNKA |
| 26063 | WLHYVEDTL |
| 26064 | NWAFNARHD |
| 26065 | CSGYWMCMS |
| 26066 | DFVLTGTIN |
| 26067 | DVNQNAKYD |
| 26068 | ITDIFHWSC |
| 26069 | HMRTMDAFE |
| 26070 | ESIQKHSVC |
| 26071 | MSEMEKQQF |
| 26072 | LMDEQPMWC |
| 26073 | HWDVQVNNA |
| 26074 | EQNHAQLMP |
| 26075 | DTAKVVELC |
| 26076 | ETSCGTLMC |
| 26077 | EQQWIAPKH |
| 26078 | EVKQATIWC |
| 26079 | TYDPFWMQH |
| 26080 | THDTTTFAF |
| 26081 | WVFQADWNT |
| 26082 | WVTPQCIHL |
| 26083 | LGHDVNGHH |
| 26084 | CHELIKKQW |
| 26085 | SNWSKCAPT |
| 26086 | LNDYTHHQA |
| 26087 | ACEQENIQH |
| 26088 | LYDYMHMGS |
| 26089 | EVPLHTVCQ |
| 26090 | VHGCGKQYH |
| 26091 | MHMDQPTMC |
| 26092 | VKFSNEMKW |
| 26093 | MELHKCNSP |
| 26094 | ELHGTYNSC |
| 26095 | SEAEMDWIG |
| 26096 | AFRTMDEVH |
| 26097 | IIDNVLCNG |
| 26098 | MKMCNCPYQ |
| 26099 | MILVQQLQN |
| 26100 | PSTGNIQFM |
| 26101 | QHPGFGLVI |
| 26102 | IWWEHMGPA |
| 26103 | QWRWHKSAC |
| 26104 | DSESRLCED |
| 26105 | ETSAEFEQV |
| 26106 | FLGSMWPRD |
| 26107 | CVTLEQSSF |
| 26108 | HMTAMCLAG |
| 26109 | NVELRHHGY |
| 26110 | MALDQRSKP |
| 26111 | DFIYDGEDM |
| 26112 | FDRHDKSCC |
| 26113 | TIGQHCADF |
| 26114 | HVANGTDAC |
| 26115 | DSHTRRYDT |
| 26116 | MIPQPVEPF |
| 26117 | TVAHHFVET |

### Example 23

Treatment of a Neurological Disease or Condition with an AAV5-Derived Virion Encapsidating a Therapeutic Payload

This example describes treatment of a neurological disease or condition with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a region from a position corresponding to 581 to a position corresponding to 589 in the VP1 capsid polypeptide and where the polynucleotide sequence encoding for the variant AAV5 Cap encodes for an amino acid sequence of any one of SEQ ID NO: 7118-SEQ ID NO: 10117. The therapeutic payload is a guide RNA targeting an mRNA encoded for by a gene implicated in the neurological disease or condition or a transgene. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared wildtype AAV5 virions encapsidating the therapeutic payload. Upon administration to the subject, a lower dose of the variant AAV5 virions encapsidating the therapeutic payload is administered as compared to wildtype AAV5 virions encapsidating the therapeutic payload. Upon administration to the subject, at least one symptom of neurological disease or condition is alleviated or the subject is cured.

### Example 24

Treatment of Alzheimer's Disease with an AAV5-Derived Virion Encapsidating a Therapeutic Payload

This example describes treatment of Alzheimer's disease with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a region from a position corresponding to 581 to a position corresponding to 589 in the VP1 capsid polypeptide and where the polynucleotide sequence encoding for the variant AAV5 Cap encodes for an amino acid sequence of any one of SEQ ID NO: 7118-SEQ ID NO: 10117. Each such variant is detargeted for all non-CNS tissues, including being detargeted for cardiac tissue. The therapeutic payload is a guide RNA targeting an mRNA encoded for by a gene implicated in Alzheimer's disease or a transgene. The mRNA targeted by the guide RNA is an mRNA encoded for by a gene encoding for amyloid precursor protein (APP), alpha-synuclein (SNCA), Tau protein (MAPT), or Apolipoprotein E (ApoE). The transgene is a gene encoding for amyloid precursor protein (APP), alpha-synuclein (SNCA), Tau protein (MAPT), or Apolipoprotein E (ApoE). The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared wildtype AAV5 virions encapsidating the therapeutic payload. Upon administration to the subject, a lower dose of the variant AAV5 virions encapsidating the therapeutic payload is administered as compared to wildtype AAV5 virions encapsidating the therapeutic payload. Upon administration to the subject, at least one symptom of Alzheimer's disease is alleviated or the subject is cured.

### Example 25

Treatment of Parkinson's Disease with an AAV5-Derived Virion Encapsidating a Therapeutic Payload

This example describes treatment of Parkinson's disease with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a region from a position corresponding to 581 to a position corresponding to 589 in the VP1 capsid polypeptide and where the polynucleotide sequence encoding for the variant AAV5 Cap encodes for an amino acid sequence of any one of SEQ ID NO: 7118-SEQ ID NO: 10117. The therapeutic payload is a guide RNA targeting an mRNA encoded for by a gene implicated in Parkinson's disease or a transgene. The mRNA targeted by the guide RNA is an mRNA encoded for by a gene encoding for leucine-rich repeat kinase 2 (LRRK2). The transgene is LRRK2; GBA1; GBA1+alpha-synuclein; AADC; GDNF; Neurturin; GAD; NTN; hFOXG1; hKCNQ2; hFMR1; anti-Tau/miRNA; EPM2A or EPM2B. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared wildtype AAV5 virions encapsidating the therapeutic payload. Upon administration to the subject, a lower dose of the variant AAV5 virions encapsidating the therapeutic payload is administered as compared to wildtype AAV5 virions encapsidating the therapeutic payload. Upon administration to the subject, at least one symptom of Parkinson's disease is alleviated or the subject is cured.

### Example 26

Treatment of a Tauopathy with an AAV5-Derived Virion Encapsidating a Therapeutic Payload

This example describes treatment of a Tauopathy with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a region from a position corresponding to 581 to a position corresponding to 589 in the VP1 capsid polypeptide and where the polynucleotide sequence encoding for the variant AAV5 Cap encodes for an amino acid sequence of any one of SEQ ID NO: 7118-SEQ ID NO: 10117. The therapeutic payload is a guide RNA targeting an mRNA encoded for by a gene implicated in the Tauopathy or a transgene. The mRNA targeted by the guide RNA is an mRNA encoded for by a gene (MAPT) encoding for Tau protein. The transgene is MAPT. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared wildtype AAV5 virions encapsidating the therapeutic payload. Upon administration to the subject, a lower dose of the variant AAV5 virions encapsidating the therapeutic payload is administered as compared to wildtype AAV5 virions encapsidating the therapeutic payload. Upon administration to the subject, at least one symptom of Tauopathy disease is alleviated or the subject is cured.

### Example 27

Treatment of Alpha-1 Antitrypsin Deficiency (AATD) with an AAV5-Derived Virion Encapsidating a Therapeutic Payload

This example describes treatment of AATD with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a region from a position corresponding to 581 to a position corresponding to 589 in the VP1 capsid polypeptide and where the polynucleotide sequence encoding for the variant AAV5 Cap encodes for an amino acid sequence of any one of SEQ ID NO: 43438-SEQ ID NO: 46437. The therapeutic payload is a guide RNA targeting an mRNA encoded for by a gene implicated in AATD or a transgene. The mRNA targeted by the guide RNA is an mRNA encoded for by a gene encoding for SERPINA1. The transgene is SERPINA1. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared wildtype AAV5 virions encapsidating the therapeutic payload. Upon administration to the subject, a lower dose of the variant AAV5 virions encapsidating the therapeutic payload is administered as compared to wildtype AAV5 virions encapsidating the therapeutic payload. Upon administration to the subject, at least one symptom of AATD is alleviated or the subject is cured.

### Example 28

Treatment of Cystic Fibrosis with an AAV5-Derived Virion Encapsidating a Therapeutic Payload

This example describes treatment of cystic fibrosis with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a region from a position corresponding to 581 to a position corresponding to 589 in the VP1 capsid polypeptide and where the polynucleotide sequence encoding for the variant AAV5 Cap encodes for an amino acid sequence of any one of SEQ ID NO: 16118-SEQ ID NO: 19117. The therapeutic payload is a guide RNA targeting an mRNA encoded for by a gene implicated in cystic fibrosis or a transgene. The mRNA targeted by the guide RNA is an mRNA encoded for by a gene encoding for CFTR. The transgene is CFTR. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared wildtype AAV5 virions encapsidating the therapeutic payload. Upon administration to the subject, a lower dose of the variant AAV5 virions encapsidating the therapeutic payload is administered as compared to wildtype AAV5 virions encapsidating the therapeutic payload. Upon administration to the subject, at least one symptom of cystic fibrosis disease is alleviated or the subject is cured.

### Example 29

Treatment of Duchenne Muscular Dystrophy with an AAV5-Derived Virion Encapsidating a Therapeutic Payload

This example describes treatment of Duchenne muscular dystrophy with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a region from a position corresponding to 581 to a position corresponding to 589 in the VP1 capsid polypeptide and where the polynucleotide sequence encoding for the variant AAV5 Cap encodes for an amino acid sequence of any one of SEQ ID NO: 25118-SEQ ID NO: 26117, SEQ ID NO: 28991-SEQ ID NO: 31990, or SEQ ID NO: 13118-SEQ ID NO: 16117. The therapeutic payload is a guide RNA targeting an mRNA encoded for by a gene implicated in Duchenne muscular dystrophy or a transgene. The mRNA targeted by the guide RNA is an mRNA encoded for by a gene encoding for DMD. The transgene is DMD. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared wildtype AAV5 virions encapsidating the therapeutic payload. Upon administration to the subject, a lower dose of the variant AAV5 virions encapsidating the therapeutic payload is administered as compared to wildtype AAV5 virions encapsidating the therapeutic payload. Upon administration to the subject, at least one symptom of Duchenne muscular dystrophy disease is alleviated or the subject is cured.

### Example 30

Treatment of Frontotemporal Dementia with an AAV5-Derived Virion Encapsidating a Therapeutic Payload

This example describes treatment of frontotemporal dementia (FTD) with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a region from a position corresponding to 581 to a position corresponding to 589 in the VP1 capsid polypeptide and where the polynucleotide sequence encoding for the variant AAV5 Cap encodes for an amino acid sequence of any one of SEQ ID NO: 7118-SEQ ID NO: 10117. The therapeutic payload is a guide RNA targeting an mRNA encoded for by a gene implicated in FTD or a transgene. The mRNA targeted by the guide RNA is an mRNA encoded for by a gene encoding for progranulin. The transgene is progranulin. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared to wildtype AAV5 virions encapsidating the therapeutic payload. Upon administration to the subject, a lower dose of the variant AAV5 virions encapsidating the therapeutic payload is administered as compared to wildtype AAV5 virions encapsidating the therapeutic payload. Upon administration to the subject, at least one symptom of FTD is alleviated or the subject is cured.

### Example 31

AAV5 Variants with Adrenal Gland Tissue Tropism as Identified by Machine Learning

This example describes preferred properties of engineered AAV5 variants that display adrenal gland tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display adrenal gland tissue tropism.

Favored biophysical properties and favored amino acid residues at each position in the 581 to 589 region of an AAV5 VP capsid polypeptide, which are associated with a higher probability of adrenal gland tissue tropism are described below. Any of the below machine learning-derived positional amino acid preferences described in TABLE 46 can be present alone or in combination with each other or in combination with any of the amino acid preferences observed and described in EXAMPLE 7, TABLE 13. Listed below in TABLE 46 are a summary of positional features shared between the top important features for adrenal gland tropism extracted from the ML models.

TABLE 46
Machine Learning-Derived Adrenal Gland Tissue Tropism Rules

Low mol mass at Xaa1
Xaa1 is selected from V, P, S, or C
Low hydropathy at Xaa1
Xaa1 is selected from T, S, W, or Y
Low hydropathy at Xaa2
Xaa2 is selected from R
Low mutability at Xaa2
Xaa2 is selected from C
Low solubility at Xaa2
Xaa2 is selected from K
Low average flexibility at Xaa3
Xaa3 is selected from W, M, or F
High solubility at Xaa3
Xaa3 is selected from M
High surface accessibility at Xaa4
Xaa4 is selected from K or R
High average flexibility at Xaa4
Xaa4 is selected from K, I, or N
Medium mutability at Xaa5
Xaa5 is selected from R or H
High goldman engelman steitz at Xaa5
Xaa5 is selected from V or L
Low hydropathy at Xaa5
Xaa5 is selected from R
High volume at Xaa5
Xaa5 is selected from Y, R or F
High solubility at Xaa6
Xaa6 is selected from Y, V, M, A, or C
Medium mutability at Xaa7
Xaa7 is selected from V, H, or R
Low solubility at Xaa7
Xaa7 is selected from R
High average flexibility at Xaa8
Xaa8 is selected from K, I, or N
High mol mass at Xaa8
Xaa8 is selected from R, or Y
High mutability at Xaa9
Xaa9 is selected from N

TABLE 47 below provides sequences of the 581 to 589 region for variant AAV VP1 capsid polypeptides that exhibited adrenal gland tissue tropism and comport to one or more of the rules provided in TABLE 46. The present disclosure, thus, provides for rAAVs composed of engineered AAV5 VP1 capsid polypeptides having a SEQ ID NO: 2, wherein the Xaa1 to Xaa9 region is any one of SEQ ID NO: 2118-SEQ ID NO: 3117, as disclosed in TABLE 47. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 47
Sequences of the 581 to 589 Region in AAV5 VP1
Capsid Polypeptide that Drive Adrenal
Gland Tissue Tropism

| SEQ ID | 581-589 |
|---|---|
| NO | Sequence |
| 2118 | AAGGFLSGC |
| 2119 | AAVQKERYN |
| 2120 | ACYFMMRQN |
| 2121 | AFGHAMKQF |
| 2122 | AIGCIQNGG |
| 2123 | AIGCVQNGG |
| 2124 | AIGGFPNGG |
| 2125 | AIGYFQNGG |
| 2126 | AILKLTKVL |
| 2127 | AILKMTKVM |
| 2128 | AILKTTKVL |
| 2129 | AIMKMTKVL |
| 2130 | ALGHRHHQC |
| 2131 | ALNKTLAEG |
| 2132 | ANMFRNGML |
| 2133 | ANMFRNGMR |
| 2134 | ANYIQYVWE |
| 2135 | AQMQYERLF |
| 2136 | ARAPNEGVI |
| 2137 | ARQWMCVHC |
| 2138 | ARTLYMINR |
| 2139 | ARVIEHKAG |
| 2140 | ASMFWHYQV |
| 2141 | ATCKHTDHL |
| 2142 | ATCYFVSSQ |
| 2143 | ATFKHTDHF |
| 2144 | ATFKHTDHW |
| 2145 | ATLKHTDHL |
| 2146 | ATNKYANAT |
| 2147 | ATVKHTDHL |
| 2148 | AVGLNERNG |
| 2149 | AVGNRHHQC |
| 2150 | AWGMQDQRY |
| 2151 | AWMCRGVYC |
| 2152 | AWMCRHVYC |
| 2153 | AWMCRNFYC |
| 2154 | AWMCRNVDC |
| 2155 | AWMCRNVHC |
| 2156 | AWMCRNVNC |
| 2157 | AWMCRNVYY |
| 2158 | AWMCRSVYC |
| 2159 | AWMCRTVYC |
| 2160 | AWMFRNVYC |
| 2161 | AWMRRNVYC |
| 2162 | AWRCRNVYC |
| 2163 | AWVCRNVYC |
| 2164 | AWVMKDQQY |
| 2165 | AWVMKNQRY |
| 2166 | AYCHRAHNG |
| 2167 | CAGIHLNEG |
| 2168 | CAGIHLNES |
| 2169 | CAGIHMNEA |
| 2170 | CARFVARWP |
| 2171 | CARFVEGWP |
| 2172 | CARFVERWH |
| 2173 | CARFVERWR |
| 2174 | CAVIHLNEA |
| 2175 | CAVPPSRYP |
| 2176 | CAVPTARYP |
| 2177 | CAVPTSRYL |
| 2178 | CAVPTSRYT |
| 2179 | CAVQKERYN |
| 2180 | CAVVMMRQP |
| 2181 | CCAKFKMCT |
| 2182 | CCEDMKIWQ |
| 2183 | CCVFRHHYC |
| 2184 | CDIDYDLGF |
| 2185 | CECIVQMAT |
| 2186 | CECLVQMGT |
| 2187 | CFDIIQRYD |
| 2188 | CFKFKFRLV |
| 2189 | CFKFKVRLA |
| 2190 | CFKSKFSWQ |
| 2191 | CFNIIERYD |
| 2192 | CGEDMKMWQ |
| 2193 | CGKDIKIWQ |
| 2194 | CGKDMQIWQ |
| 2195 | CHGRVAHHE |
| 2196 | CHLRDCSPI |
| 2197 | CHMDYDLGF |
| 2198 | CHRFIQPSY |
| 2199 | CHRFIQRSY |
| 2200 | CHRYIQQSY |
| 2201 | CHSRDCSPL |
| 2202 | CIKSMFSWQ |
| 2203 | CILQTRKGN |
| 2204 | CKIGTQKGQ |
| 2205 | CKIVAQKGQ |
| 2206 | CKIVIQKGQ |
| 2207 | CKIVTQKSQ |
| 2208 | CKLVTQKGQ |
| 2209 | CKMVTQKGQ |
| 2210 | CKMYMHEAR |
| 2211 | CKMYMQESR |
| 2212 | CKMYMRESR |
| 2213 | CLMALNRSG |
| 2214 | CLMAMKRSG |
| 2215 | CLMGLKRSG |
| 2216 | CLSPFARAM |
| 2217 | CLVALKRSG |
| 2218 | CMKRYMMLP |
| 2219 | CPDEPKWFP |
| 2220 | CRKQGHGEK |
| 2221 | CSIVRWMSP |
| 2222 | CSKFIVAWE |
| 2223 | CSKFIVGWE |
| 2224 | CSKFIVVWE |
| 2225 | CSSFRWMSP |
| 2226 | CSSVRWMGP |
| 2227 | CSSVRWMST |
| 2228 | CTFVMMRQP |
| 2229 | CTGQRGFTN |
| 2230 | CTGVMMRQP |
| 2231 | CTIRGNSSC |
| 2232 | CTLRGNASC |
| 2233 | CTVGMMRQP |
| 2234 | CTVLMMRQP |
| 2235 | CTVLMTRQP |
| 2236 | CTVVLMRQP |
| 2237 | CTVVMMREP |
| 2238 | CTVVMVRQP |
| 2239 | CVDTYRMWQ |
| 2240 | CVHQRMHVT |
| 2241 | CVIQTSRGT |
| 2242 | CVKFKVRLV |
| 2243 | CVLEFSRQW |
| 2244 | CVLKFSREW |
| 2245 | CVLKFSRHW |
| 2246 | CVLKFSRQG |
| 2247 | CVLKFSRQS |
| 2248 | CVLKFSRRW |
| 2249 | CVLKVSRQW |
| 2250 | CVLKYSRQW |
| 2251 | CVLQTSRGI |
| 2252 | CVMQTSRGI |
| 2253 | CVNKLSYEN |
| 2254 | CVRHTENWR |
| 2255 | CVSLFARAM |
| 2256 | CVSPFARAK |
| 2257 | CVSPFARAT |
| 2258 | CVSPFARGM |
| 2259 | CVSPFARSM |
| 2260 | CVSPFARVM |
| 2261 | CVSPFGRAM |
| 2262 | CVSPFVRAM |
| 2263 | CVSQFARAM |
| 2264 | CVSSFARAM |
| 2265 | CVTTRHKQG |
| 2266 | CVWPFKFDF |
| 2267 | CVWPMKFDF |
| 2268 | CWDVTCQEN |
| 2269 | CYGRVAHLE |
| 2270 | CYRFIQQSY |
| 2271 | CYVFRHHDC |
| 2272 | DASSNDRNT |
| 2273 | DCESPEKQN |
| 2274 | DCESSEKPN |
| 2275 | DCQSPEKPN |
| 2276 | DEDVCKFVF |
| 2277 | DEVACKFVF |
| 2278 | DEVDCKFVF |
| 2279 | DEVVCKFMF |
| 2280 | DEVVCKIVF |
| 2281 | DFKLFHNRE |
| 2282 | DFKLFHNRP |
| 2283 | DFKLFNNRA |
| 2284 | DFKVFHNRA |
| 2285 | DFQLFHNRA |
| 2286 | DFRLFHNRA |
| 2287 | DFRWFIRMN |
| 2288 | DMKRPTRYN |
| 2289 | DMQRPPRYN |
| 2290 | DMQRPSRYN |
| 2291 | DNNMIVRCV |
| 2292 | DNQWMNSLE |
| 2293 | DNSMIVRCA |
| 2294 | DQPHLHRIG |
| 2295 | DRCLVEWVS |
| 2296 | DRCLVKWVA |
| 2297 | DRMDFHFFF |
| 2298 | DSKQIAKAM |
| 2299 | DVCNVLGWK |
| 2300 | DVDVVARPM |
| 2301 | DVKLFHNRA |
| 2302 | DVRGLNREY |
| 2303 | DVRNFFDHC |
| 2304 | DWHQYDGCE |
| 2305 | DWNQHDGCE |
| 2306 | DWNQYAGCE |
| 2307 | DWNQYDGGE |
| 2308 | DWNQYNGCE |
| 2309 | DWSQYDGCE |
| 2310 | DYKQTAKAM |
| 2311 | ECAHVIKFN |
| 2312 | ECHKMRCLE |
| 2313 | ECHKMRCVE |
| 2314 | ECNKMRCIE |
| 2315 | EETKKVKHW |
| 2316 | EHKKNINMH |
| 2317 | EKRHISRAP |
| 2318 | EQRHISRAR |
| 2319 | EQRHISRTP |
| 2320 | ERVIEHRAG |
| 2321 | ERVLEHKAG |
| 2322 | EYDVNMRQM |
| 2323 | FGKDAFVSS |
| 2324 | FGKDTFASS |
| 2325 | FGKDTFLSS |
| 2326 | FGKDTFVSA |
| 2327 | FGKDTVVSS |
| 2328 | FGMDTFVSS |
| 2329 | FMMHRVMSD |
| 2330 | FMMHRVMSG |
| 2331 | FMMMAKTQA |
| 2332 | FQKLRDVCF |
| 2333 | FREQGHGEK |
| 2334 | FRKEGHGEK |
| 2335 | FRKPGHGEK |
| 2336 | FRKQAHGEK |
| 2337 | FRKQGHGAK |
| 2338 | FRKQGHGEE |
| 2339 | FRKQGHGQK |
| 2340 | FRKQGNGEK |
| 2341 | FRKQGPGEK |
| 2342 | FRKQVHGEK |
| 2343 | FRNQVHGEK |
| 2344 | FSSCCRQKG |
| 2345 | FTSNRNWEA |
| 2346 | FTSNRNWQD |
| 2347 | FTSNRNWQP |
| 2348 | GAHHRCRMT |
| 2349 | GAWMQGFHP |
| 2350 | GCAKFQMCT |
| 2351 | GCAKTRFEN |
| 2352 | GCDKNRFEN |
| 2353 | GCDKTRFAN |
| 2354 | GCDKTRFES |
| 2355 | GCDKTRFEY |
| 2356 | GCHKMRCIE |
| 2357 | GEGIRKMNN |
| 2358 | GEGIRQMNT |
| 2359 | GEGVRQMNN |
| 2360 | GEVVCKFVF |
| 2361 | GFDRQDAHR |
| 2362 | GFKLFHNRA |
| 2363 | GMKTRCDDD |
| 2364 | GMKTRCDYA |
| 2365 | GMKTRCDYV |
| 2366 | GMKTRCHYD |
| 2367 | GMKTRCNYD |
| 2368 | GMKTRCVYD |
| 2369 | GMQTRCDYD |
| 2370 | GMRTRCDYD |
| 2371 | GRAFVKIDT |
| 2372 | GRICMQGMK |
| 2373 | GRIFMLGMK |
| 2374 | GRIFVKIDT |
| 2375 | GRLFVKIDT |
| 2376 | GRWFQIGTE |
| 2377 | GTFKHTDHL |
| 2378 | GVAYRESQA |
| 2379 | GVDKLSYEN |
| 2380 | GVHHRWRMT |
| 2381 | GVKPRWGLF |
| 2382 | GVKTRCDYD |
| 2383 | GVWMQGIHP |
| 2384 | GWEKKINFQ |
| 2385 | GWEKKINGQ |
| 2386 | GWEKKLNVQ |
| 2387 | GWEKKVNVQ |
| 2388 | GWEKTINVQ |
| 2389 | GWFFKNYYC |
| 2390 | GWKKKINVQ |
| 2391 | GWMCRNVYC |
| 2392 | GWQKKINVQ |
| 2393 | HACCLLQQY |
| 2394 | HACSLLKQY |
| 2395 | HACSLLQQF |
| 2396 | HIEWVSMQR |
| 2397 | HIQWVSMHR |
| 2398 | HMEWVSMHR |
| 2399 | HRADIMCWN |
| 2400 | HREAIMCWN |
| 2401 | HREDIMCGN |
| 2402 | HREDIMCWD |
| 2403 | HREDIMCWI |
| 2404 | HREDIRCWN |
| 2405 | HRGDIMCWN |
| 2406 | HRKDIMCWN |
| 2407 | HRQDIMCWN |
| 2408 | HRTETECWC |
| 2409 | HSCSLLQQY |
| 2410 | HVGALTRSD |
| 2411 | IARHIDHDL |
| 2412 | IAWHVDHDL |
| 2413 | ICHLRHIAA |
| 2414 | ICHLRHIPV |
| 2415 | ICHLRHISG |
| 2416 | ICHLRHNAG |
| 2417 | ICHLRHVAG |
| 2418 | ICTTLIQGM |
| 2419 | ICTTQIKGM |
| 2420 | ICYKWWVMF |
| 2421 | IDAALDMSW |
| 2422 | IEVKYWNRH |
| 2423 | IEYVRSCPW |
| 2424 | IFMAFDHLE |
| 2425 | IGMDKPVDA |
| 2426 | IHMKPQESP |
| 2427 | IHMKQQDSP |
| 2428 | IHMKQQESA |
| 2429 | IHMKQQEST |
| 2430 | IHMKQQQSP |
| 2431 | IHMKQRESP |
| 2432 | IHMKTIVGE |
| 2433 | IHMRQQESP |
| 2434 | IILHNMKSW |
| 2435 | IMLALMHED |
| 2436 | IMLHRVMSD |
| 2437 | IMMAHWFAH |
| 2438 | IMMAQWFAQ |
| 2439 | IMMAQWFGH |
| 2440 | IMMAQWFSH |
| 2441 | IMMAQWIAH |
| 2442 | IMMHRMMSD |
| 2443 | IMMHRVMSE |
| 2444 | IMMHRVMSV |
| 2445 | IMMHRVMSY |
| 2446 | IMMHRVMYD |
| 2447 | IMMLRVMSD |
| 2448 | IMMNRVMSD |
| 2449 | IMMQRVMSD |
| 2450 | IMMVQWFAH |
| 2451 | IMMYRVMSD |
| 2452 | IMVALMHEG |
| 2453 | IVCRMWHQP |
| 2454 | IWHKWWVMF |
| 2455 | IWYKWWAMF |
| 2456 | IWYKWWLMF |
| 2457 | IWYKWWVMC |
| 2458 | IWYKWWVMV |
| 2459 | IWYKWWVMY |
| 2460 | KAIPENSNR |
| 2461 | KALEVHFQK |
| 2462 | KAMEFHFQK |
| 2463 | KAMPEPVTE |
| 2464 | KAQDFATEN |
| 2465 | KAQEFATED |
| 2466 | KAQEFATEH |
| 2467 | KAQKFATEN |
| 2468 | KAQQFATEN |
| 2469 | KAREFATEN |
| 2470 | KASTEAGDK |
| 2471 | KATTEASDK |
| 2472 | KCDKPMLEP |
| 2473 | KCDKPMLQP |
| 2474 | KCDTFRGME |
| 2475 | KCEPFRGME |
| 2476 | KCETFLGME |
| 2477 | KCGILYYQG |
| 2478 | KCGNLYYQG |
| 2479 | KCGSLNYQG |
| 2480 | KCGSLYCQG |
| 2481 | KCGSLYHQG |
| 2482 | KCGSLYSQG |
| 2483 | KCGSLYYQV |
| 2484 | KCGSMYYQG |
| 2485 | KCGTLYYQG |
| 2486 | KCYFMMRQN |
| 2487 | KDCQYIGAC |
| 2488 | KDCQYIGSW |
| 2489 | KDCQYLGAW |
| 2490 | KFELNQTAV |
| 2491 | KFELNRKAV |
| 2492 | KFETFRGME |
| 2493 | KFGLNQKAV |
| 2494 | KFIDVWDAR |
| 2495 | KGEYQADAP |
| 2496 | KHDPMCGQL |
| 2497 | KHEQMCVQL |
| 2498 | KHERMCVQL |
| 2499 | KHETMCVQL |
| 2500 | KHKINAVER |
| 2501 | KHKPMCVQL |
| 2502 | KHKYEDPMV |
| 2503 | KHKYEDTMG |
| 2504 | KHQINTVER |
| 2505 | KHQPMCVQL |
| 2506 | KHSSHMEAC |
| 2507 | KIHYHFEDK |
| 2508 | KINYHFEDE |
| 2509 | KINYHFEDQ |
| 2510 | KISGLNDAT |
| 2511 | KKDEANGQT |
| 2512 | KKDPMQTYP |
| 2513 | KMGTEVSQQ |
| 2514 | KMRMIFRKD |
| 2515 | KNEPMCVQL |
| 2516 | KPVEYASAP |
| 2517 | KQDFHHGLA |
| 2518 | KQLYHIPTC |
| 2519 | KQQYVTMNA |
| 2520 | KQRDISRAP |
| 2521 | KQRHISRAP |
| 2522 | KQSAHMEAC |
| 2523 | KQSPHMEAC |
| 2524 | KQSSHMEAF |
| 2525 | KRDLANGQF |
| 2526 | KRIIEHPCA |
| 2527 | KRMDFHFFF |
| 2528 | KRSSHMEAC |
| 2529 | KRVIEHKAG |
| 2530 | KRYFWMSYY |
| 2531 | KSMLEDRIC |
| 2532 | KSMWEARIC |
| 2533 | KSMWEDRIG |
| 2534 | KSMWEDRTC |
| 2535 | KSMWEERIC |
| 2536 | KSMWENRIC |
| 2537 | KSMWEYRIC |
| 2538 | KSVWEDRIC |
| 2539 | KTFFWMSYY |
| 2540 | KTFSESTCR |
| 2541 | KTMEVHFQK |
| 2542 | KTQWDEAIV |
| 2543 | KTYFADTIC |
| 2544 | KTYFADTIS |
| 2545 | KTYFADTMG |
| 2546 | KTYFADTVG |
| 2547 | KTYFAETIG |
| 2548 | KTYFANTIG |
| 2549 | KTYFSDTIG |
| 2550 | KTYFWMSYC |
| 2551 | KTYFWMSYS |
| 2552 | KTYFWTSYY |
| 2553 | KTYFWVSYY |
| 2554 | KVDVFTVKD |
| 2555 | KVDVITFKD |
| 2556 | KVDVITVQD |
| 2557 | KVDVVTVKD |
| 2558 | KVILDSFRR |
| 2559 | KVMPEPVPE |
| 2560 | KVMPEPVTA |
| 2561 | KVMPEPVTV |
| 2562 | KVMPVPVTE |
| 2563 | KVMSEPVTE |
| 2564 | KVTWMDLED |
| 2565 | KVVLDSFRQ |
| 2566 | KVYMEPANW |
| 2567 | KYEFLIKYI |
| 2568 | KYEFLLKHI |
| 2569 | KYEFLLKYL |
| 2570 | KYEFLLRYI |
| 2571 | KYEILNCNP |
| 2572 | KYEVLLKYI |
| 2573 | KYEYLLKYI |
| 2574 | KYIAVWDAR |
| 2575 | KYIDFWDAR |
| 2576 | KYIDIWDAR |
| 2577 | KYIDVWEAR |
| 2578 | KYKAPTINP |
| 2579 | KYKHPTINP |
| 2580 | KYKPLTINP |
| 2581 | KYKPPTMNP |
| 2582 | KYKRPTINP |
| 2583 | KYQPPTINP |
| 2584 | LAVDKSRLL |
| 2585 | LCGLMPRPA |
| 2586 | LCGVMPRPG |
| 2587 | LFKPEDEDR |
| 2588 | LFKPEDPDR |
| 2589 | LFKPEEQDR |
| 2590 | LHRDPMPIM |
| 2591 | LHRDQMPFM |
| 2592 | LHRDQMPLM |
| 2593 | LIMAHWLHM |
| 2594 | LIMAYWLDM |
| 2595 | LIMSHWLDM |
| 2596 | LKRPQGEAE |
| 2597 | LLMAHWLDM |
| 2598 | LQKYYMNQP |
| 2599 | LQNYRDEDG |
| 2600 | LQPWWLGMI |
| 2601 | LQTYHHVDG |
| 2602 | LQTYQPVDG |
| 2603 | LRFLSDADP |
| 2604 | LRKEGHGEK |
| 2605 | LRYLSDTDP |
| 2606 | LSTNRLVNA |
| 2607 | LSTNRMVSA |
| 2608 | LSTNRMVTA |
| 2609 | LYRDQMPIM |
| 2610 | MCGHASKAV |
| 2611 | MCGHASRAE |
| 2612 | MCGPASKAE |
| 2613 | MCHLRHIAG |
| 2614 | MCRANGNMP |
| 2615 | MCSLRHWQP |
| 2616 | MIGMWNRSN |
| 2617 | MISMLNRSN |
| 2618 | MRSIYMNIS |
| 2619 | MRWILTIME |
| 2620 | MSAQRHVQT |
| 2621 | MSAQRYAQT |
| 2622 | MSAQRYVQA |
| 2623 | MSMLRMHEA |
| 2624 | NAMCFGRAR |
| 2625 | NAMCFGREK |
| 2626 | NAMCFGRTK |
| 2627 | NAMCIGRAK |
| 2628 | NAMGFGRAK |
| 2629 | NATCFGRAK |
| 2630 | NCNKCINRT |
| 2631 | NCNKCKHRT |
| 2632 | NCNKCKYRT |
| 2633 | NCRHTILYV |
| 2634 | NCSKCAKYD |
| 2635 | NCVKLGCQS |
| 2636 | NELQDQCGM |
| 2637 | NEVVCKFVF |
| 2638 | NFSKCAKYV |
| 2639 | NFSKCTKYD |
| 2640 | NFSKGAKYD |
| 2641 | NGMDKAVDA |
| 2642 | NGMDKPFDA |
| 2643 | NGMDKPIDA |
| 2644 | NGMDKPVDG |
| 2645 | NGMDKPVDS |
| 2646 | NGMDKPVNA |
| 2647 | NGMDQPVDA |
| 2648 | NGMDRPVDA |
| 2649 | NGMDTPVDA |
| 2650 | NGVDKPVDA |
| 2651 | NGWESEQHM |
| 2652 | NHMKQQESP |
| 2653 | NLDMSRRAF |
| 2654 | NLDMSRRDC |
| 2655 | NMIKRCCQG |
| 2656 | NMIKRCSKG |
| 2657 | NMIKRCSQA |
| 2658 | NMIKRCSQC |
| 2659 | NMIKRCSQS |
| 2660 | NMIKRCSQV |
| 2661 | NMIKWCSQG |
| 2662 | NMVKRCSQG |
| 2663 | NNQWKNSLE |
| 2664 | NNQWTNSLE |
| 2665 | NPCKINVGT |
| 2666 | NQCKINFGT |
| 2667 | NQCKINVGN |
| 2668 | NQCKINVGP |
| 2669 | NQCKLNVGT |
| 2670 | NQLKDVAAS |
| 2671 | NQWKINVGT |
| 2672 | NRAETECWC |
| 2673 | NRCLVEWVA |
| 2674 | NRMDFHFFV |
| 2675 | NRMDFNFFF |
| 2676 | NRNKCKNRT |
| 2677 | NRPFNYFQR |
| 2678 | NRTLYMINR |
| 2679 | NSSYFEFSN |
| 2680 | NSVKLGCQN |
| 2681 | NTIKRCSQG |
| 2682 | NVDMSRRDF |
| 2683 | NVMCFGRAK |
| 2684 | NVRNFFAHC |
| 2685 | NVRNFFEHC |
| 2686 | NVSKCAKYD |
| 2687 | NVWPNIAEM |
| 2688 | NVWTNISEM |
| 2689 | NWNGRWNQC |
| 2690 | NWNKCKNRT |
| 2691 | NWPCHDNRH |
| 2692 | NWSYHDNRH |
| 2693 | NWTGRWHQC |
| 2694 | NYCDRAHNG |
| 2695 | PAVQKERYN |
| 2696 | PCFSIARYY |
| 2697 | PCYFMMRQN |
| 2698 | PKVTCRYRM |
| 2699 | PPLHSRCGM |
| 2700 | PQLHSRCRM |
| 2701 | PQLLSRCGM |
| 2702 | PQLNSRCGM |
| 2703 | PQLRSRCGM |
| 2704 | PWVMKDQRY |
| 2705 | QAYCCMHQD |
| 2706 | QCDEFKMRM |
| 2707 | QCHKMRCIE |
| 2708 | QESNRCWCD |
| 2709 | QHKERMLFD |
| 2710 | QHKERMVFD |
| 2711 | QHQKNINMH |
| 2712 | QLDEFKMRM |
| 2713 | QMSWVQNWE |
| 2714 | QMYHNHHTA |
| 2715 | QMYWAQNWE |
| 2716 | QMYWGQNWE |
| 2717 | QMYWIQNWE |
| 2718 | QMYWVQNLE |
| 2719 | QMYWVQSWE |
| 2720 | QPVTRMTNE |
| 2721 | QQLHSRCGM |
| 2722 | QQRHISRAP |
| 2723 | QRDLAHGQF |
| 2724 | QRDLANGKS |
| 2725 | QRDLANGLF |
| 2726 | QRDLANGQI |
| 2727 | QRDLANGQS |
| 2728 | QRDLANGQV |
| 2729 | QRDLANGQY |
| 2730 | QRDMANGQF |
| 2731 | QRDPANGQF |
| 2732 | QSAFRQSDS |
| 2733 | QSTFRQCDS |
| 2734 | QSTMRQYYA |
| 2735 | QSYCCMHQY |
| 2736 | QTYWVQNWE |
| 2737 | QVSGMFHPA |
| 2738 | QVYCCMHQY |
| 2739 | QVYWVQNWE |
| 2740 | QWDEFKKRM |
| 2741 | QWDEFKMRR |
| 2742 | QWDEFKVRM |
| 2743 | QWDVFKMRM |
| 2744 | RACSLLQQY |
| 2745 | RCDKTRFEN |
| 2746 | RCEAFNWRE |
| 2747 | RCELFNWRE |
| 2748 | RCEPFHWRE |
| 2749 | RCEPFNLRE |
| 2750 | RCEPFNWHE |
| 2751 | RCGHASKAE |
| 2752 | RCGPFNWRE |
| 2753 | RCGQVRNEM |
| 2754 | RCKLFDHDE |
| 2755 | RCVEDHFHK |
| 2756 | RCVLDHFHK |
| 2757 | RCVQDHFHE |
| 2758 | RCVQDHFHR |
| 2759 | RCVQDHFHT |
| 2760 | RCVQDHFNK |
| 2761 | RCVQDHIHK |
| 2762 | RCVQDHSHK |
| 2763 | RCVQDNFHK |
| 2764 | RCVQHHFHK |
| 2765 | RCVRDHFHK |
| 2766 | RCYFMMRQN |
| 2767 | RDLSLHLMR |
| 2768 | RECLIERCG |
| 2769 | RECLIERCI |
| 2770 | RECLIKRCS |
| 2771 | RECLVERCS |
| 2772 | RIERVDVEN |
| 2773 | RKDEANGQA |
| 2774 | RMDEANGQT |
| 2775 | RQCLIERCS |
| 2776 | RQMWMAHHA |
| 2777 | RRDAKVANG |
| 2778 | RRDEANGQT |
| 2779 | RREDIMCWN |
| 2780 | RRKDIMKER |
| 2781 | RRYRELTEE |
| 2782 | RSEPFNWRE |
| 2783 | RTDAIHHQV |
| 2784 | RTDAIHPQI |
| 2785 | RWVQDHFHK |
| 2786 | RYCQVWGCA |
| 2787 | RYDLFSYHQ |
| 2788 | RYDLVSHHQ |
| 2789 | RYDLVSYHR |
| 2790 | RYDLVSYYQ |
| 2791 | RYNLVSYHQ |
| 2792 | RYRQEWGCA |
| 2793 | SAGQKERYN |
| 2794 | SAIQKERYN |
| 2795 | SALYGSKAI |
| 2796 | SAVQQERYN |
| 2797 | SAVQRERYN |
| 2798 | SAVRNERYN |
| 2799 | SAWYGSKSI |
| 2800 | SCKGFHLQN |
| 2801 | SCKSHQMHE |
| 2802 | SCRHIYREA |
| 2803 | SCRHLYHEA |
| 2804 | SCRHNYHEA |
| 2805 | SCRRIYHEA |
| 2806 | SFDRQDVHR |
| 2807 | SFDRQGAHR |
| 2808 | SFGHAMKQC |
| 2809 | SFKGFHLEN |
| 2810 | SFKGFHVQN |
| 2811 | SFMGFHLQN |
| 2812 | SFVRQDAHR |
| 2813 | SGQKFIFCP |
| 2814 | SIGCFQNGG |
| 2815 | SIIFSNTCM |
| 2816 | SILLPGIQG |
| 2817 | SIMHEGVQS |
| 2818 | SIMLPGIQA |
| 2819 | SLNQAIHLE |
| 2820 | SMGPTVHEM |
| 2821 | SMNQAVHLE |
| 2822 | SMNQPIHLE |
| 2823 | SMRSQHADY |
| 2824 | SNCALHNAW |
| 2825 | SNCAQHNSW |
| 2826 | SNCAQYNAW |
| 2827 | SNCSQHNAW |
| 2828 | SNCVQHNAW |
| 2829 | SNGAQHNAW |
| 2830 | SNGFEWACE |
| 2831 | SNGFEWVCA |
| 2832 | SNGFEWVGE |
| 2833 | SNGFEWVSE |
| 2834 | SNGFEWVYE |
| 2835 | SNMFRNGMQ |
| 2836 | SNVFEWVCE |
| 2837 | SRDEAKSWV |
| 2838 | SRICMLGMK |
| 2839 | SRIILDYYS |
| 2840 | SRPFNYIQR |
| 2841 | SRSFNYFQR |
| 2842 | SRWCQIGTE |
| 2843 | SRWFKIGTE |
| 2844 | SRWFQIGTK |
| 2845 | SSGDRRSDS |
| 2846 | SSKGFHLQN |
| 2847 | SSKQDFYGL |
| 2848 | SSKSHRMHE |
| 2849 | SSMLEGVQS |
| 2850 | SSVDRRSDG |
| 2851 | SSVHALYFP |
| 2852 | SSVHTLYYP |
| 2853 | SSVQKERYN |
| 2854 | STCYFVASL |
| 2855 | STDPYTSHH |
| 2856 | STFKHTDHL |
| 2857 | STGFEWVCE |
| 2858 | STSYFVASQ |
| 2859 | SVGPTVHQM |
| 2860 | SVSKFQADK |
| 2861 | SVVQKERYN |
| 2862 | SWEKKINVQ |
| 2863 | SWGLIHGWN |
| 2864 | SWGLIHWWN |
| 2865 | SWGLIHYWN |
| 2866 | SWGLVHCWN |
| 2867 | SWKWDKYCN |
| 2868 | SWMCRNVYC |
| 2869 | SWRHIYHEA |
| 2870 | SWRTRGNFE |
| 2871 | SWSKCNMHV |
| 2872 | SWVLIHCWN |
| 2873 | SYDRQDAHR |
| 2874 | SYVFRHHYC |
| 2875 | TACATYRSF |
| 2876 | TACMTYRSF |
| 2877 | TACVAYRSF |
| 2878 | TACVNYRSF |
| 2879 | TACVTYRGL |
| 2880 | TACVTYRIF |
| 2881 | TAFVTYRSF |
| 2882 | TAFVTYRSL |
| 2883 | TAGHEGHSC |
| 2884 | TAGHQGHIC |
| 2885 | TAGVTYRSF |
| 2886 | TAITMWQYM |
| 2887 | TAMCFGRAK |
| 2888 | TAMSMCMYG |
| 2889 | TAMTICMYG |
| 2890 | TANDHRVDA |
| 2891 | TATDRRVDA |
| 2892 | TAVQKERYN |
| 2893 | TAVSICMYG |
| 2894 | TAVTMWQSM |
| 2895 | TAWVTYRSF |
| 2896 | TAYVTYRSF |
| 2897 | TCCFMMRQN |
| 2898 | TCCSIARYY |
| 2899 | TCDFMMRQN |
| 2900 | TCFFMMRQN |
| 2901 | TCFGIARYY |
| 2902 | TCFNIARYY |
| 2903 | TCFSIARCY |
| 2904 | TCFSIARYD |
| 2905 | TCFSIARYF |
| 2906 | TCFSIARYS |
| 2907 | TCFSIDRYY |
| 2908 | TCFSIGRYY |
| 2909 | TCFSITRYY |
| 2910 | TCFSIVRYY |
| 2911 | TCFSNARYY |
| 2912 | TCFSTARYY |
| 2913 | TCFSVARYY |
| 2914 | TCFTIARYY |
| 2915 | TCFTVARYY |
| 2916 | TCGSLYYQG |
| 2917 | TCHFALHGC |
| 2918 | TCHFMMRQN |
| 2919 | TCHYALHAC |
| 2920 | TCNKCKNRT |
| 2921 | TCRYALHGC |
| 2922 | TCSFMMRQN |
| 2923 | TCSRFDNQA |
| 2924 | TCVSIARYY |
| 2925 | TCWALFRGE |
| 2926 | TCWDLFRGG |
| 2927 | TCYFMMGQN |
| 2928 | TCYFMMREN |
| 2929 | TCYFMMRKN |
| 2930 | TCYFMMRPN |
| 2931 | TCYFMMRQD |
| 2932 | TCYFMMRQK |
| 2933 | TCYFMMRQT |
| 2934 | TCYFMMRRN |
| 2935 | TCYFMVRQN |
| 2936 | TCYSIARYY |
| 2937 | TCYSMMRQN |
| 2938 | TCYVMMRQN |
| 2939 | TCYYMMRQN |
| 2940 | TDCVTYRSF |
| 2941 | TFDLSNKVT |
| 2942 | TFGWFMRIP |
| 2943 | TFKGFHLQN |
| 2944 | TFSKCAKYD |
| 2945 | TFYFMMRQN |
| 2946 | TGCVTYRSF |
| 2947 | TGGLNERNG |
| 2948 | TGGTFVRTM |
| 2949 | TGMDKPVDA |
| 2950 | TGQATERAL |
| 2951 | TGTDHRVDA |
| 2952 | TGWSTCMIP |
| 2953 | TITKPINLF |
| 2954 | TITKPSHLF |
| 2955 | TLNKTLAES |
| 2956 | TNTCECVDR |
| 2957 | TNYIQYVWD |
| 2958 | TPLMMCRAS |
| 2959 | TPMMMCQAS |
| 2960 | TRCYFVASQ |
| 2961 | TRDAKVSNG |
| 2962 | TREWMCVHC |
| 2963 | TRHMGVSAG |
| 2964 | TRKWMCVHC |
| 2965 | TRLWMCVHC |
| 2966 | TRMDFHFFF |
| 2967 | TRPWMCVHC |
| 2968 | TRQRMCVHC |
| 2969 | TRQWMCAHC |
| 2970 | TRQWMCIHC |
| 2971 | TRQWMCLHC |
| 2972 | TRQWMCVHG |
| 2973 | TRQWMCVHW |
| 2974 | TRQWMCVYC |
| 2975 | TRQWMGVHC |
| 2976 | TRQWMWVHC |
| 2977 | TRTLYMIDR |
| 2978 | TRTLYRINR |
| 2979 | TRTPYMINR |
| 2980 | TRWFQIGTE |
| 2981 | TRYFMMRQN |
| 2982 | TSCDRAHNA |
| 2983 | TSCVTYRSF |
| 2984 | TTCFFVASQ |
| 2985 | TTCFIVASQ |
| 2986 | TTCYFVASE |
| 2987 | TTCYFVASP |
| 2988 | TTCYFVASW |
| 2989 | TTCYFVGSQ |
| 2990 | TTCYFVSSQ |
| 2991 | TTCYFVVSQ |
| 2992 | TTCYIVASE |
| 2993 | TTCYLVASP |
| 2994 | TTFKHTDHL |
| 2995 | TTGYFVASQ |
| 2996 | TTRYFVASQ |
| 2997 | TTWYFVASQ |
| 2998 | TTYYFVASQ |
| 2999 | TVGLNERNA |
| 3000 | TVGLNERNS |
| 3001 | TVGLNKRNG |
| 3002 | TVGVNERNG |
| 3003 | TVKMHFAAP |
| 3004 | TVKTNFTSK |
| 3005 | TVQSNFTSK |
| 3006 | TWALAAKYE |
| 3007 | TWFSIARYY |
| 3008 | TWKWDEYCN |
| 3009 | TWKWDKYCH |
| 3010 | TWKWDKYYN |
| 3011 | TWKWDRYCN |
| 3012 | TWNKCNMHV |
| 3013 | TWPYHDNRH |
| 3014 | TWQTKDFYN |
| 3015 | TWSKCNLHV |
| 3016 | TWSKCNMRV |
| 3017 | TWSKCNVHV |
| 3018 | TWSKCSMHV |
| 3019 | TWSKCTMHV |
| 3020 | TWSKFNMHV |
| 3021 | TWSRCNMHV |
| 3022 | TWTYCEQRH |
| 3023 | TYCDRADNG |
| 3024 | TYCDRAHDV |
| 3025 | TYCDRAHNA |
| 3026 | TYCDRTHNG |
| 3027 | TYFDRAHTG |
| 3028 | TYWDRAHNG |
| 3029 | TYYDRAHNG |
| 3030 | VACRMWHQP |
| 3031 | VAEKRMDRS |
| 3032 | VARHVDHDL |
| 3033 | VAWSNEDQK |
| 3034 | VCHTFLRLG |
| 3035 | VFCRMWHQP |
| 3036 | VGEKRMDRS |
| 3037 | VHEQLEGCK |
| 3038 | VILKMTKVL |
| 3039 | VLGNRHHQC |
| 3040 | VLKKFGHSF |
| 3041 | VLMQYERLF |
| 3042 | VLNKTLAES |
| 3043 | VLPKFGHSF |
| 3044 | VLQKFGHSV |
| 3045 | VLRKFGHSF |
| 3046 | VMKTRCDYD |
| 3047 | VMMHRVMSD |
| 3048 | VPIEYMTEK |
| 3049 | VPIPYMTEK |
| 3050 | VPIQYMAEK |
| 3051 | VPMQYMTEK |
| 3052 | VQEQLESCK |
| 3053 | VQKLRDLCC |
| 3054 | VQMQYARLF |
| 3055 | VQMQYERLY |
| 3056 | VQMQYQRLF |
| 3057 | VQMQYVRLF |
| 3058 | VQTYQHVDG |
| 3059 | VQVQYERLF |
| 3060 | VRIQYMTEK |
| 3061 | VRMQYERLF |
| 3062 | VSAECYFDM |
| 3063 | VSDEFYFDM |
| 3064 | VSDVCYFDM |
| 3065 | VSGECYFDM |
| 3066 | VTWMVRTSN |
| 3067 | VVCRMWYQP |
| 3068 | VVEKRMERS |
| 3069 | VVFRMWHQP |
| 3070 | VVWMQGFHP |
| 3071 | VVYRMWHQP |
| 3072 | VWRYFCVSL |
| 3073 | VWYKWWVMF |
| 3074 | WATKWDVHR |
| 3075 | WCNTRLAMA |
| 3076 | WCTSRLAMA |
| 3077 | WCTTRLTMA |
| 3078 | WCTTRLVMA |
| 3079 | WWMHGRGGR |
| 3080 | YACSLLQQY |
| 3081 | YAVQKERYN |
| 3082 | YCHKTAVAA |
| 3083 | YCHQQDRIT |
| 3084 | YCNKTAVAA |
| 3085 | YCYKNAVAA |
| 3086 | YCYKTAVAV |
| 3087 | YCYRTAVAA |
| 3088 | YGEAQITSP |
| 3089 | YIKQICADA |
| 3090 | YIKQICSDS |
| 3091 | YINKNPHQM |
| 3092 | YLHKNPHQM |
| 3093 | YLNKNHHQM |
| 3094 | YLNKNLHQM |
| 3095 | YLNKNPHQR |
| 3096 | YLNKNPHQV |
| 3097 | YLNQNPHQM |
| 3098 | YLTKNPHQM |
| 3099 | YMIKRCSQG |
| 3100 | YPTQRLHHL |
| 3101 | YRDEAKCWV |
| 3102 | YRDEAKSWA |
| 3103 | YRDEAQSWV |
| 3104 | YRDEDKSWV |
| 3105 | YRGEAKSWV |
| 3106 | YRHEAKSWV |
| 3107 | YRKQGHGEK |
| 3108 | YRMEYGTCK |
| 3109 | YRMEYGTWE |
| 3110 | YSAQLRQQM |
| 3111 | YSVDRRSDS |
| 3112 | YWCPNQWWR |
| 3113 | YWCPSKWWR |
| 3114 | YWCPSQLWR |
| 3115 | YWCPSQWLR |
| 3116 | YWGLIHCWN |
| 3117 | YWGPSQWWR |

### Example 32

AAV5 Variants with Bone Marrow Tissue Tropism as Identified by Machine Learning

This example describes preferred properties of engineered AAV5 variants that display bone marrow tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display bone marrow tissue tropism.

Favored biophysical properties and favored amino acid residues at each position in the 581 to 589 region of an AAV5 VP capsid polypeptide, which are associated with a higher probability of bone marrow tissue tropism are described below. Any of the below machine learning-derived positional amino acid preferences described in TABLE 48 can be present alone or in combination with each other or in combination with any of the amino acid preferences observed and described in EXAMPLE 18, TABLE 35. Listed below in TABLE 48 are a summary of positional features shared between the top important features for bone marrow tropism extracted from the ML models.

TABLE 48
Machine Learning-Derived Bone Marrow Tissue Tropism Rules

High hydropathy at Xaa1
Xaa1 is selected from V, I, or L
Low mutability at Xaa1
Xaa1 is selected from Y, L, F, or C
Low hydropathy at Xaa2
Xaa2 is selected from Y or W
High mol mass at Xaa2
Xaa2 is selected from W
Low surface accessibility at Xaa2
Xaa2 is selected from W or A
Low hydrophilicity at Xaa2
Xaa2 is selected from W
Low mutability at Xaa2
Xaa2 is selected from C
Low average flexibility at Xaa2
Xaa2 is selected from W, M, or F
Low average flexibility at Xaa5
Xaa5 is selected from W, M, or F
Low average flexibility at Xaa6
Xaa6 is selected from W, M, or F
Low mutability at Xaa6
Xaa6 is selected from Y, F, L, or C
High solubility at Xaa6
Xaa6 is selected from W, F, I, or L
Low surface accessibility at Xaa7
Xaa7 is selected from C
Or
High surface accessibility at Xaa7
Xaa7 is selected from D or N
Low mutability at Xaa7
Xaa7 is selected from C
High solubility at Xaa7
Xaa7 is selected from C
Or
Low solubility at Xaa7
Xaa7 is selected from D
Low charge at Xaa8
Xaa8 is selected from D or E
High mutability at Xaa8
Xaa8 is selected from D, E, A, or T
High mol mass at Xaa9
Xaa9 is selected from H or F
Low mutability at Xaa9
Xaa9 is selected from Y, F, or L

TABLE 49 below provides sequences of the 581 to 589 region for variant AAV VP1 capsid polypeptides that exhibited bone marrow tissue tropism and comport to one or more of the rules provided in TABLE 48. The present disclosure, thus, provides for rAAVs composed of engineered AAV5 VP1 capsid polypeptides having a SEQ ID NO: 2, wherein the Xaa1 to Xaa9 region is any one of SEQ ID NO: 5118-SEQ ID NO: 6117, as disclosed in TABLE 49. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 49
Sequences of the 581 to 589 Region in AAV5 VP1
Capsid Polypeptide that Drive Bone
Marrow Tissue Tropism

| SEQ ID | 581-589 |
|---|---|
| NO | Sequence |
| 5118 | AADVVPCHH |
| 5119 | ACFHVAEMF |
| 5120 | AEMHMVADF |
| 5121 | AESLCNELC |
| 5122 | AKKSCQCWH |
| 5123 | ANDYDQCCM |
| 5124 | ANGHKFNEE |
| 5125 | ANNEFIQPF |
| 5126 | ANQCIFKTM |
| 5127 | AQCLCNELC |
| 5128 | AQGLCNELC |
| 5129 | AQHDWYQKY |
| 5130 | AQPNMDYVP |
| 5131 | AQPSMDCVP |
| 5132 | AQPSMDYAP |
| 5133 | AQPSMDYVA |
| 5134 | AQPSMEYVP |
| 5135 | AQSLCNELF |
| 5136 | AQSLCNELG |
| 5137 | AQSLCNQLC |
| 5138 | AQSLFNELC |
| 5139 | AQSVCNELC |
| 5140 | AQTLCNELC |
| 5141 | ARAKIQDEW |
| 5142 | ARALIQDEW |
| 5143 | ARARIQDEW |
| 5144 | ARGQIQDEW |
| 5145 | ARPSMDYVP |
| 5146 | ARVQIQDEW |
| 5147 | AWESSSIIS |
| 5148 | AYAENWCIE |
| 5149 | CANHMIRTR |
| 5150 | CATHMIRTK |
| 5151 | CCEHKEYLN |
| 5152 | CCEPKGYLN |
| 5153 | CCRKDNKKS |
| 5154 | CCRMANKKS |
| 5155 | CCRMDDKKS |
| 5156 | CCRMDHKKS |
| 5157 | CCRMDIKKS |
| 5158 | CCRMDNKES |
| 5159 | CCRMDNKIS |
| 5160 | CCRMDNKKA |
| 5161 | CCRMDNKRS |
| 5162 | CCRMDNQKS |
| 5163 | CCRRDNKKS |
| 5164 | CDAEAEFQR |
| 5165 | CDAEDEFRR |
| 5166 | CDAKAEFRR |
| 5167 | CDAVAEFRR |
| 5168 | CDDEAEFRR |
| 5169 | CDGEAEFLR |
| 5170 | CDGEAEFRR |
| 5171 | CDGVAEFRR |
| 5172 | CFKQKHNLF |
| 5173 | CFLQKHNLF |
| 5174 | CFQEKHNLF |
| 5175 | CFQPKHNLF |
| 5176 | CFQQKHNIF |
| 5177 | CFQQKHNLI |
| 5178 | CFQQNHNLF |
| 5179 | CFQRKHNLF |
| 5180 | CGYHEFMVG |
| 5181 | CHCDAVECF |
| 5182 | CHFDAMECF |
| 5183 | CHKCIGQDN |
| 5184 | CHQCIGPDN |
| 5185 | CHQCIGQDS |
| 5186 | CHQCIGQDT |
| 5187 | CHQCVGQDN |
| 5188 | CHWDAMECF |
| 5189 | CIKIQLIDQ |
| 5190 | CIKIQMIDK |
| 5191 | CIKIRMIDQ |
| 5192 | CIKVQMIDQ |
| 5193 | CINITAITP |
| 5194 | CISITVITP |
| 5195 | CMAHIWEAH |
| 5196 | CMAHIWEGD |
| 5197 | CMDHIWEGH |
| 5198 | CMSHIWEGH |
| 5199 | CPQCIGQDN |
| 5200 | CRCDAMECF |
| 5201 | CWYDEFMVG |
| 5202 | CWYHEFMVA |
| 5203 | CWYHEFMVD |
| 5204 | CWYHEFMVR |
| 5205 | CWYHEFMVS |
| 5206 | CWYHEFMVV |
| 5207 | CWYHEFVVG |
| 5208 | CWYHEHMVG |
| 5209 | CWYHEVMVG |
| 5210 | CWYHKFMVG |
| 5211 | CWYLEFMVG |
| 5212 | CWYNEFMVG |
| 5213 | CWYPEFMVG |
| 5214 | CWYREFMVG |
| 5215 | CWYYEFMVG |
| 5216 | DACDWKNDY |
| 5217 | DACQEWDSE |
| 5218 | DADFCARMP |
| 5219 | DADFCPRMA |
| 5220 | DADFCPRMS |
| 5221 | DADFCPRMT |
| 5222 | DARFDQWHY |
| 5223 | DCAENWCIE |
| 5224 | DCIHDQCSS |
| 5225 | DDDCWVFYH |
| 5226 | DDDFWVFYH |
| 5227 | DDDHWVFYH |
| 5228 | DDDYWAFYH |
| 5229 | DDDYWGFYH |
| 5230 | DDGSWVFYH |
| 5231 | DFAENWCIE |
| 5232 | DGDYWVFYH |
| 5233 | DGGCWPREC |
| 5234 | DHAENWCIE |
| 5235 | DHELTGSEA |
| 5236 | DITQLQCYC |
| 5237 | DKNEMFMKW |
| 5238 | DLDCDWLHS |
| 5239 | DMDCEWLHS |
| 5240 | DPCQDWDSE |
| 5241 | DPCQEWNSE |
| 5242 | DPCQKWDSE |
| 5243 | DPCQVWDSE |
| 5244 | DPCREWDSE |
| 5245 | DPGQEWDSE |
| 5246 | DQCQEWDSE |
| 5247 | DRCQEWDSE |
| 5248 | DSCQEWDSE |
| 5249 | DSRFDQWHH |
| 5250 | DTCQEWDSE |
| 5251 | DYAEHWCIE |
| 5252 | DYAEIWCIE |
| 5253 | DYAEKWCIA |
| 5254 | DYAENGCIE |
| 5255 | DYAENWCIA |
| 5256 | DYAENWCIG |
| 5257 | DYAENWCME |
| 5258 | DYAENWCNE |
| 5259 | DYAENWCVE |
| 5260 | DYAENWWIE |
| 5261 | DYAETWCIE |
| 5262 | DYAQNWCIE |
| 5263 | DYAVNWCIE |
| 5264 | DYDENWCIE |
| 5265 | DYGENWCIE |
| 5266 | DYPENWCIE |
| 5267 | DYTENWCIE |
| 5268 | DYVENWCIE |
| 5269 | EAEDEIQKS |
| 5270 | EAEDEIQRA |
| 5271 | EAGDEIQKA |
| 5272 | EATGMLYEE |
| 5273 | EAWNAHEEG |
| 5274 | ECESSSIIS |
| 5275 | ECIHAQCSS |
| 5276 | ECIHDKCSS |
| 5277 | ECIHDQCCS |
| 5278 | ECIHDQCSA |
| 5279 | ECIHDQCSP |
| 5280 | ECIHDQCST |
| 5281 | ECIHDQCTS |
| 5282 | ECIHHQCSS |
| 5283 | ECIHVQCSS |
| 5284 | EDFHEFAQT |
| 5285 | EDGHKFNEE |
| 5286 | EEKSCQCWH |
| 5287 | EGIHDQCSS |
| 5288 | EHAEWKCMY |
| 5289 | EHHEWKCMY |
| 5290 | EHPEGKCMY |
| 5291 | EHPELKCMY |
| 5292 | EHPEWKCLC |
| 5293 | EHPEWKGMY |
| 5294 | EHPEWTCMY |
| 5295 | EHREWKCMY |
| 5296 | EHSEWKCMY |
| 5297 | EHTEWKCMY |
| 5298 | EINQSTCET |
| 5299 | EKDQSTCET |
| 5300 | EKESCQCWH |
| 5301 | EKHQSTCET |
| 5302 | EKIMVKRPF |
| 5303 | EKIMVKRTF |
| 5304 | EKIMVKWAF |
| 5305 | EKKACQCWH |
| 5306 | EKKPCQCWH |
| 5307 | EKKSCKCWH |
| 5308 | EKKSCLCWH |
| 5309 | EKKSCPCWH |
| 5310 | EKKSCQCWD |
| 5311 | EKKSCQCWN |
| 5312 | EKKSCRCWH |
| 5313 | EKKSFQCWH |
| 5314 | EKKSWQCWH |
| 5315 | EKKSYQCWH |
| 5316 | EKKTCQCWH |
| 5317 | EKLLSLCPS |
| 5318 | EKNESTCET |
| 5319 | EKNQCTCET |
| 5320 | EKNQGTCET |
| 5321 | EKNQSTCEA |
| 5322 | EKNQSTCEP |
| 5323 | EKNQSTCES |
| 5324 | EKNQSTCGT |
| 5325 | EKNVWNNKW |
| 5326 | EKQSCQCWH |
| 5327 | EKRSCQCWH |
| 5328 | EKTQSTCET |
| 5329 | EKTSCQCWH |
| 5330 | ELPEWKCMY |
| 5331 | ENAHKFNEE |
| 5332 | ENGDKFNEE |
| 5333 | ENGHEFNEE |
| 5334 | ENGHKFDEE |
| 5335 | ENGHKFNAE |
| 5336 | ENGHKFNEA |
| 5337 | ENGHKFNEK |
| 5338 | ENGHKFNKE |
| 5339 | ENGHKFNQE |
| 5340 | ENGHKFSEE |
| 5341 | ENGHKFTEE |
| 5342 | ENGHQFNEE |
| 5343 | ENGHRFNEE |
| 5344 | ENPEWKCMY |
| 5345 | ENVHKFNEE |
| 5346 | EQHDWCQKY |
| 5347 | EQHDWYKKY |
| 5348 | EQHDWYLKY |
| 5349 | EQHHWYQKY |
| 5350 | EQHNWYQKY |
| 5351 | EQKSCQCWH |
| 5352 | EQLLSLCHS |
| 5353 | EQLLSLCLS |
| 5354 | EQLLSLCPN |
| 5355 | EQLLSLCSS |
| 5356 | EQLLTLCPS |
| 5357 | EQLSSLCPS |
| 5358 | EQNDWYQKY |
| 5359 | EQPLSLCPS |
| 5360 | EQVLSLCPS |
| 5361 | ERIHDQCSS |
| 5362 | ERKSCQCWH |
| 5363 | ERNQSTCET |
| 5364 | ESEDEIQKA |
| 5365 | ESGHKFNEE |
| 5366 | ESIGMVCSD |
| 5367 | ETALKWEWS |
| 5368 | ETAWKWDWS |
| 5369 | ETGHKFNEE |
| 5370 | ETSWKWEWS |
| 5371 | EVPWESKLC |
| 5372 | EVWDAHEEG |
| 5373 | EVWNSHEEG |
| 5374 | EWEGSSIIS |
| 5375 | EWESNSIIS |
| 5376 | EWESSSIIA |
| 5377 | EWESSSTIS |
| 5378 | EWETSSIIS |
| 5379 | EWGFGSDET |
| 5380 | EWIHDQCSS |
| 5381 | EWLPWINER |
| 5382 | FEMAMCSMG |
| 5383 | FKIRKFWDP |
| 5384 | FKMAMCSMG |
| 5385 | FKVGKFWDP |
| 5386 | FKVIKFWDP |
| 5387 | FKVRKFLDP |
| 5388 | FKVRKFWDS |
| 5389 | FQMAMGSMG |
| 5390 | FQMSMCSMG |
| 5391 | FQVRKFWDP |
| 5392 | FRIIWGADM |
| 5393 | FRILWQCEG |
| 5394 | FRMAMCSMG |
| 5395 | FRPIWGADM |
| 5396 | FRPIWGGDM |
| 5397 | FRTIWGADL |
| 5398 | FRTIWGADV |
| 5399 | FRTIWGPDM |
| 5400 | FRTIWGTDM |
| 5401 | FRVRKFWDP |
| 5402 | FVDSMLHKW |
| 5403 | FWDRQWDDF |
| 5404 | FWLGLFPTK |
| 5405 | FWLGVFPAK |
| 5406 | FWMGLFPAK |
| 5407 | GATHMIRTR |
| 5408 | GCEAKEYLN |
| 5409 | GCLTWAFRG |
| 5410 | GCVTWAFRC |
| 5411 | GCVTWDFRG |
| 5412 | GDHDMLNSL |
| 5413 | GEQEARNCS |
| 5414 | GEREGRNCS |
| 5415 | GEYLRHTVT |
| 5416 | GEYLRNTIT |
| 5417 | GEYLRSTVT |
| 5418 | GEYQRNTVT |
| 5419 | GGAAMWGRY |
| 5420 | GGAAMWRKY |
| 5421 | GGAGMWRRY |
| 5422 | GGASMWRRY |
| 5423 | GGATMWRRY |
| 5424 | GGDAMWRRY |
| 5425 | GGEERVGEA |
| 5426 | GGGAMWRRY |
| 5427 | GIEGMYWPL |
| 5428 | GILIMAYSW |
| 5429 | GLDCEWLHS |
| 5430 | GLLYDFNPE |
| 5431 | GLLYDFNQG |
| 5432 | GLPHSADVI |
| 5433 | GLPQSADGI |
| 5434 | GLPRSADVI |
| 5435 | GLSVMTYTS |
| 5436 | GMHHAVWAS |
| 5437 | GMNHAVGAS |
| 5438 | GMNHAVWAN |
| 5439 | GMNHAVWTS |
| 5440 | GMNHSVWAS |
| 5441 | GMNNAVWAS |
| 5442 | GMNQAVWAS |
| 5443 | GMNYAVWAS |
| 5444 | GMPQSADVI |
| 5445 | GMSHAVWAS |
| 5446 | GMTHAVWAS |
| 5447 | GNGHKFNEE |
| 5448 | GQSLCNELC |
| 5449 | GVPQSADVI |
| 5450 | GWMCEVPWT |
| 5451 | GWMWEVHWT |
| 5452 | GWMWEVPWR |
| 5453 | GWMWEVTWT |
| 5454 | HDEMMGLER |
| 5455 | HDIMFAHQM |
| 5456 | HDIMFSHQM |
| 5457 | HDIMFTHKM |
| 5458 | HDIMFTHKR |
| 5459 | HDIMFTHQR |
| 5460 | HDIMFTYQM |
| 5461 | HDKMMGLER |
| 5462 | HDLMFTHQM |
| 5463 | HDQMMDLER |
| 5464 | HDQMMGMER |
| 5465 | HNIMFTHQM |
| 5466 | IADVVPCHH |
| 5467 | IAMLESWWS |
| 5468 | ICFHVAEMF |
| 5469 | IDMLESWWG |
| 5470 | IGEAQDCNP |
| 5471 | IGMIRATRE |
| 5472 | IGMIRDARE |
| 5473 | IGMIRDTHE |
| 5474 | IGMIWDTRE |
| 5475 | IGMLRDTRE |
| 5476 | IGVMLNAMK |
| 5477 | IGVMLNDMQ |
| 5478 | ILHMMHRGA |
| 5479 | IMAPLVCDD |
| 5480 | IMAPLVCDG |
| 5481 | IMEALVCDD |
| 5482 | IMEHLVCDD |
| 5483 | IMEHMVCDD |
| 5484 | IMEPLACDD |
| 5485 | IMEPLVCAD |
| 5486 | IMEPLVCDA |
| 5487 | IMEPLVCDN |
| 5488 | IMEPLVCDV |
| 5489 | IMEPLVCGD |
| 5490 | IMEPLVCND |
| 5491 | IMEPPVCDD |
| 5492 | IMEPRVCDD |
| 5493 | IMEPVVCDD |
| 5494 | IMERLVCDD |
| 5495 | IMERMVCDD |
| 5496 | IMKPLVCDD |
| 5497 | IMKQWPAMF |
| 5498 | IMPQWPAMF |
| 5499 | IMQEWPAMF |
| 5500 | IMQPLVCDD |
| 5501 | IMQPWPAMF |
| 5502 | IMQQWLAMF |
| 5503 | IMQQWPAKF |
| 5504 | IMQQWPAMY |
| 5505 | IMQQWPDMF |
| 5506 | IMQQWPSMF |
| 5507 | IMQQWPTMF |
| 5508 | IMRQWPAMF |
| 5509 | IMVPLVCDD |
| 5510 | INNEFIQPF |
| 5511 | IPDLMHNVH |
| 5512 | IPGLMSDYQ |
| 5513 | IRQQWPAMF |
| 5514 | IRTIWGADM |
| 5515 | ISALMSDYQ |
| 5516 | ISDLMHDVH |
| 5517 | ISDLMHNAH |
| 5518 | ISDLMHNGH |
| 5519 | ISDLMHNVN |
| 5520 | ISDLMHSVH |
| 5521 | ISDLMHYVH |
| 5522 | ISDLMNNVH |
| 5523 | ISDLVHNVH |
| 5524 | ISDMMHNVH |
| 5525 | ISELMSDYQ |
| 5526 | ISGLMSDDQ |
| 5527 | ISGLMSDHQ |
| 5528 | ISGLMSDYE |
| 5529 | ISGLMSDYK |
| 5530 | ISGLTSDYQ |
| 5531 | ISGLVSDYQ |
| 5532 | ISNLMHNVH |
| 5533 | ISQHTCNKD |
| 5534 | ISQHTCSED |
| 5535 | ISQQTCSKD |
| 5536 | ISRHTCSKD |
| 5537 | ISSADSWPM |
| 5538 | ISVLMSDYQ |
| 5539 | ITCIEAQEN |
| 5540 | ITCIEGLEN |
| 5541 | ITCIEGQED |
| 5542 | ITCIEGREN |
| 5543 | ITCLEGQEN |
| 5544 | ITEPLVCDD |
| 5545 | ITQHTCSKD |
| 5546 | IWARQWDDF |
| 5547 | IWDHQWDDF |
| 5548 | IWDMMHRGA |
| 5549 | IWDRKWDDF |
| 5550 | IWDRLWDDF |
| 5551 | IWDRPWDDF |
| 5552 | IWDRQLDDF |
| 5553 | IWDRQWADF |
| 5554 | IWDRQWDAF |
| 5555 | IWDRQWDDY |
| 5556 | IWDRQWGDF |
| 5557 | IWDRQWNDF |
| 5558 | IWDRQWYDF |
| 5559 | IWDSQWDDF |
| 5560 | IWHRQWDDF |
| 5561 | IWKLPSVTT |
| 5562 | IWKMPPIDM |
| 5563 | IWMLPSITT |
| 5564 | IWMLPSVAT |
| 5565 | IWMLPSVIT |
| 5566 | IWVRQWDDF |
| 5567 | IWYRQWDDF |
| 5568 | KCCLGKNSR |
| 5569 | KFHSKERMS |
| 5570 | KINIFAYTY |
| 5571 | KINIFEYTC |
| 5572 | KINVFEYTY |
| 5573 | KITIFEYTY |
| 5574 | KKDVWNNKW |
| 5575 | KKHVWNNKW |
| 5576 | KKNAWNNKW |
| 5577 | KKNEMFMKW |
| 5578 | KKNEWNNKW |
| 5579 | KKNGWNNKW |
| 5580 | KKNVWDNKW |
| 5581 | KKNVWHNKW |
| 5582 | KKNVWNNEW |
| 5583 | KKNVWNSKW |
| 5584 | KKNVWNTKW |
| 5585 | KKNVWNYKW |
| 5586 | KKNVWSNKW |
| 5587 | KKNVWYNKW |
| 5588 | KKSVWNNKW |
| 5589 | KKYVWNNKW |
| 5590 | KLLPWINER |
| 5591 | KLNIFEYTY |
| 5592 | KNDHFVCYL |
| 5593 | KNGHKFNEE |
| 5594 | KNNEFIQPF |
| 5595 | KPVQCACEN |
| 5596 | KQHDWYQKY |
| 5597 | KRLPWINER |
| 5598 | KTNVWNNKW |
| 5599 | KWGFGSDET |
| 5600 | KWLPCINER |
| 5601 | KWLPGINER |
| 5602 | KWLPWIHER |
| 5603 | KWLPWIKER |
| 5604 | KWLPWISER |
| 5605 | KWLPWVNER |
| 5606 | KWLQWINER |
| 5607 | KWLRWINER |
| 5608 | KWMPWINER |
| 5609 | KWPPWINER |
| 5610 | KWQPWINER |
| 5611 | KWRPWINER |
| 5612 | LADSMLHKW |
| 5613 | LAGQMDEMS |
| 5614 | LAIQPCDER |
| 5615 | LATGMLYEE |
| 5616 | LDDSMLHKW |
| 5617 | LDQMMGLER |
| 5618 | LGIQPCDER |
| 5619 | LIDSMLHKW |
| 5620 | LILIMAYSW |
| 5621 | LPAWQQHGR |
| 5622 | LQGGFDHLK |
| 5623 | LQGGFDHLP |
| 5624 | LQGGFDHLS |
| 5625 | LSASMEYEE |
| 5626 | LSGLIDEMS |
| 5627 | LSGQIAEMS |
| 5628 | LSGQIDEMA |
| 5629 | LSGQIDEMP |
| 5630 | LSGQIDEMT |
| 5631 | LSGQIDEMY |
| 5632 | LSMVFSRGE |
| 5633 | LSVQIDEMS |
| 5634 | LSVVFSKGE |
| 5635 | LVDIMLHKW |
| 5636 | LVDSMLHEW |
| 5637 | LVDSMLHRW |
| 5638 | LVDSMLRKW |
| 5639 | LVDSMPHKW |
| 5640 | LVDTMLHKW |
| 5641 | LVHSMLHKW |
| 5642 | LVIEPCDER |
| 5643 | LVIKPCDER |
| 5644 | LVIQACDER |
| 5645 | LVIQHCDER |
| 5646 | LVIQPCAER |
| 5647 | LVIQPCDEG |
| 5648 | LVIQPCDEH |
| 5649 | LVIQPCDEP |
| 5650 | LVIQPCDKR |
| 5651 | LVIQPCNER |
| 5652 | LVIQPYDER |
| 5653 | LVIQSCDER |
| 5654 | LVIRPCDER |
| 5655 | LVLQPCDER |
| 5656 | LVMQPCDER |
| 5657 | LVNSMLHKW |
| 5658 | LVTQPCDER |
| 5659 | LVVQPCDER |
| 5660 | LWAAMEYEE |
| 5661 | LWAFLEYEE |
| 5662 | LWAFMEYEE |
| 5663 | LWASMAYEE |
| 5664 | LWASMEFEE |
| 5665 | LWASMEHEE |
| 5666 | LWASMESEE |
| 5667 | LWASMEYDK |
| 5668 | LWASMEYEA |
| 5669 | LWASMEYEQ |
| 5670 | LWASMEYEV |
| 5671 | LWASMGYEE |
| 5672 | LWASMKYEE |
| 5673 | LWASVEYEE |
| 5674 | LWENYHSEF |
| 5675 | LWEQWCWEF |
| 5676 | LWESKEMCS |
| 5677 | LWGSMEYEE |
| 5678 | LWKEWCWEF |
| 5679 | LWKKWCWEF |
| 5680 | LWKMPPIDM |
| 5681 | LWKPRHYDF |
| 5682 | LWKPWCWEF |
| 5683 | LWKQGCWEF |
| 5684 | LWKQLCWEF |
| 5685 | LWKQSCWEF |
| 5686 | LWKQWCGEF |
| 5687 | LWKQWCLEF |
| 5688 | LWKQWCSEF |
| 5689 | LWKQWCWEC |
| 5690 | LWKQWCWEI |
| 5691 | LWKQWCWES |
| 5692 | LWKQWCWEV |
| 5693 | LWKQWCWEY |
| 5694 | LWKQWCWGF |
| 5695 | LWKQWCWKF |
| 5696 | LWKQWFWEF |
| 5697 | LWKQWGWEC |
| 5698 | LWKRWCWEF |
| 5699 | LWMQWCWEF |
| 5700 | LWPSMEYEE |
| 5701 | LWQQWCWEF |
| 5702 | LWRQWCWEF |
| 5703 | LWTQWCWEF |
| 5704 | LWTSMEYEV |
| 5705 | LWVRQWDDF |
| 5706 | LWVSMEYEE |
| 5707 | LYEIRFSDM |
| 5708 | LYEIRFVDM |
| 5709 | LYKIRFADM |
| 5710 | LYKQAPSEG |
| 5711 | LYMPAPSEG |
| 5712 | LYMQATSEG |
| 5713 | LYVIRFADM |
| 5714 | LYVQAPSEG |
| 5715 | MAEPMGCQA |
| 5716 | MAEQMGCRA |
| 5717 | MAEQMVCQA |
| 5718 | MAGDCACAM |
| 5719 | MDHPNDHFF |
| 5720 | MDHTNDLFF |
| 5721 | MEWQSEYVS |
| 5722 | MGEQMGCQA |
| 5723 | MGEVMDFLP |
| 5724 | MKNVWNNKW |
| 5725 | MLGHANDGL |
| 5726 | MLGHASDAL |
| 5727 | MMEPLVCDD |
| 5728 | MSAVMDFLP |
| 5729 | MSEIMDFLP |
| 5730 | MSEVMDFMP |
| 5731 | MSEVMDFPP |
| 5732 | MSEVMDFQP |
| 5733 | MSEVMDFRP |
| 5734 | MSGVMDFLP |
| 5735 | MSQVMDFLP |
| 5736 | MSWNWWALY |
| 5737 | MSWNWWNLY |
| 5738 | MSWNWWTVY |
| 5739 | MSWTWWTLY |
| 5740 | MTEQMGCQA |
| 5741 | MWASMEYEE |
| 5742 | MWDRQWDDF |
| 5743 | MWKQWCWEF |
| 5744 | NACDLKNDY |
| 5745 | NACDWKSDY |
| 5746 | NACDWKTDY |
| 5747 | NACDWKYDY |
| 5748 | NACDWRNDY |
| 5749 | NADVVPCHH |
| 5750 | NAYDWKNDY |
| 5751 | NCFHVAEMF |
| 5752 | NFFDSEDMG |
| 5753 | NGGCWAREC |
| 5754 | NGGCWLREC |
| 5755 | NGGCWPWEC |
| 5756 | NGGCWSREC |
| 5757 | NGGCWTREC |
| 5758 | NGGFWPREC |
| 5759 | NGGGWPREC |
| 5760 | NINQLQCYC |
| 5761 | NITQLQCCC |
| 5762 | NKHEMFMKW |
| 5763 | NKNAMFMKW |
| 5764 | NKNGMFMKW |
| 5765 | NKNVWNNKW |
| 5766 | NLDCEWLHS |
| 5767 | NMEPLVCDD |
| 5768 | NMTQLQCYC |
| 5769 | NNDDFVCYL |
| 5770 | NNDHCVCYL |
| 5771 | NNDHFICYL |
| 5772 | NNDHFLCYL |
| 5773 | NNDHFVCFL |
| 5774 | NNDHFVCHL |
| 5775 | NNDHFVCNL |
| 5776 | NNDHFVCYM |
| 5777 | NNDHIVCYL |
| 5778 | NNDHSVCYL |
| 5779 | NNDHVVCYL |
| 5780 | NNDHYVCYL |
| 5781 | NNDRFVCYL |
| 5782 | NNHHFVCYL |
| 5783 | NPCQEWDSE |
| 5784 | NRGMNAWTA |
| 5785 | NSCDWKNDY |
| 5786 | NTCIEGQEN |
| 5787 | NVGQITQDC |
| 5788 | NWFDSEDMG |
| 5789 | NYAENWCIE |
| 5790 | NYRVHGGAG |
| 5791 | NYRVHGSAA |
| 5792 | NYRVRGSAG |
| 5793 | PATGMLYEE |
| 5794 | PKELFPTCM |
| 5795 | PLCHVMADR |
| 5796 | PMDWVAECR |
| 5797 | PNNEFIQPF |
| 5798 | PQCHVMDDR |
| 5799 | PQCHVMSDR |
| 5800 | PQCHVTADR |
| 5801 | PQCLVMADR |
| 5802 | PQCRVMADR |
| 5803 | PQSLCNELC |
| 5804 | PQYHVMADR |
| 5805 | PVIQPCDER |
| 5806 | PWGFGSDET |
| 5807 | PWKIDFWYH |
| 5808 | PWKVDFWDH |
| 5809 | PWLSEFMHW |
| 5810 | QAAGMLYEE |
| 5811 | QANGMLYEE |
| 5812 | QATRMLYEE |
| 5813 | QATVMLYEE |
| 5814 | QDFHEFAET |
| 5815 | QDFHEFAPT |
| 5816 | QDFHEFAQA |
| 5817 | QDFHEFDQT |
| 5818 | QDFHEFPQT |
| 5819 | QDFPEFAQT |
| 5820 | QDIHEFAQT |
| 5821 | QFMCEIECR |
| 5822 | QFMFEIECK |
| 5823 | QHPEWKCMY |
| 5824 | QKGISEFQR |
| 5825 | QKNVWNNKW |
| 5826 | QMDWVAGCR |
| 5827 | QMDWVGECR |
| 5828 | QNCFFMHWW |
| 5829 | QNGHKFNEE |
| 5830 | QNWFFLHWW |
| 5831 | QPTGMLYEE |
| 5832 | QQCHVMADR |
| 5833 | QQHDWYQKY |
| 5834 | QVPWESRLC |
| 5835 | QVTGMLYEE |
| 5836 | QVWNAHEEG |
| 5837 | QWAFGSDET |
| 5838 | QWESKEMGS |
| 5839 | QWESKEMYS |
| 5840 | QWGCGSDET |
| 5841 | QWGFASDET |
| 5842 | QWGFESDET |
| 5843 | QWGFGADET |
| 5844 | QWGFGSDEA |
| 5845 | QWGFGSDEN |
| 5846 | QWGFGSDEP |
| 5847 | QWGFGSGET |
| 5848 | QWGFGSHET |
| 5849 | QWGFGSNET |
| 5850 | QWGFGSVET |
| 5851 | QWGFVSDET |
| 5852 | QWGIGSDET |
| 5853 | QWGSGSDET |
| 5854 | QWGVGSDET |
| 5855 | QWGYGSDET |
| 5856 | QWKQWCWEF |
| 5857 | QWVFGSDET |
| 5858 | RDFHEFAQT |
| 5859 | RDIMFTHQM |
| 5860 | RHELLEWDS |
| 5861 | RHQLLEWAS |
| 5862 | RHQLMEWDS |
| 5863 | RHQLVEWDS |
| 5864 | RHRLLEWDS |
| 5865 | RKNVWNNKW |
| 5866 | RMDWVAECR |
| 5867 | RNNEFIQPF |
| 5868 | RRQLLEWDS |
| 5869 | RSSTFEHHK |
| 5870 | RSTTFERHK |
| 5871 | RVIQPCDER |
| 5872 | RVTRDERWG |
| 5873 | RWGFGSDEA |
| 5874 | RWKQWCWEF |
| 5875 | SCFHVAEMF |
| 5876 | SCILRLISS |
| 5877 | SCYSHICRI |
| 5878 | SFDPSWWMY |
| 5879 | SFFLRLISS |
| 5880 | SFHPIWWMY |
| 5881 | SFHPSWWMS |
| 5882 | SFHPSWWVY |
| 5883 | SFHPTWWMY |
| 5884 | SFHRSWWMY |
| 5885 | SFHSSWWMY |
| 5886 | SFHTSWWMY |
| 5887 | SFILRLICS |
| 5888 | SFILRLISF |
| 5889 | SFILRPISS |
| 5890 | SFNPSWWMY |
| 5891 | SFNSHICRI |
| 5892 | SFSSHICRI |
| 5893 | SFYNHICRI |
| 5894 | SFYPSWWMY |
| 5895 | SFYSHICHI |
| 5896 | SFYSHICPI |
| 5897 | SFYSHICRM |
| 5898 | SFYSLICRI |
| 5899 | SFYSNICRI |
| 5900 | SFYSPICRI |
| 5901 | SFYSYICRI |
| 5902 | SGFPMWDYW |
| 5903 | SGIPMWDHW |
| 5904 | SGIPMWDYR |
| 5905 | SGIPMWEYW |
| 5906 | SGIPMWNYW |
| 5907 | SGIPVWDYW |
| 5908 | SGIQMWDYW |
| 5909 | SGIRMWDYW |
| 5910 | SGNPMWDYW |
| 5911 | SILIMAYSW |
| 5912 | SIYSHICRI |
| 5913 | SMNSLWHMR |
| 5914 | SQSLCNELC |
| 5915 | SRAQIQDEW |
| 5916 | SRTIWGADM |
| 5917 | SSILRLISS |
| 5918 | SSIPMWDYW |
| 5919 | SVPMDHCWA |
| 5920 | SWDRQWDDF |
| 5921 | SWKIDFWDH |
| 5922 | SYAVLQRTM |
| 5923 | SYILRLISS |
| 5924 | SYYSHICRI |
| 5925 | TADAVPCHH |
| 5926 | TADVVLCHH |
| 5927 | TADVVPCDH |
| 5928 | TAEAQDCNP |
| 5929 | TAEVVPCHH |
| 5930 | TANVVPCHH |
| 5931 | TCEAQDCNP |
| 5932 | TCEGREVDS |
| 5933 | TCEGRKLDS |
| 5934 | TCFDSEDMG |
| 5935 | TCFHAAEMF |
| 5936 | TCFHFAEMF |
| 5937 | TCFHGAEMF |
| 5938 | TCFHIAEMF |
| 5939 | TCFHVAAMF |
| 5940 | TCFHVDEMF |
| 5941 | TCFHVTEMF |
| 5942 | TCFNVAEMF |
| 5943 | TCFPVAEMF |
| 5944 | TCFRVAEMF |
| 5945 | TCGRDEFAF |
| 5946 | TCGRDLFAF |
| 5947 | TCGRDQCAF |
| 5948 | TCGRDQFAY |
| 5949 | TCGRDQFSF |
| 5950 | TCIHVAEMF |
| 5951 | TFFHVAEMF |
| 5952 | TFHPSWWMY |
| 5953 | TGDVVPCHH |
| 5954 | TGEAEDCNP |
| 5955 | TGEAQDCNA |
| 5956 | TGEAQDCSP |
| 5957 | TGFHVAEMF |
| 5958 | TGGCWPREC |
| 5959 | TGIPMWDYW |
| 5960 | TGKAQDCNP |
| 5961 | TGMSELADE |
| 5962 | TGPMDHCWA |
| 5963 | THFPWSGEA |
| 5964 | THQLLEWDS |
| 5965 | TMEPLVCDD |
| 5966 | TMSVVQCER |
| 5967 | TNDEFIQPF |
| 5968 | TNDHFVCYL |
| 5969 | TNHCIFKTM |
| 5970 | TNHEFIQPF |
| 5971 | TNIEFIQPF |
| 5972 | TNMSELADE |
| 5973 | TNNAVIQPF |
| 5974 | TNNECIQPF |
| 5975 | TNNEFFQPF |
| 5976 | TNNEFIEPF |
| 5977 | TNNEFIQLF |
| 5978 | TNNEFIQQF |
| 5979 | TNNEFIQRF |
| 5980 | TNNEFIQSF |
| 5981 | TNNEFIRPF |
| 5982 | TNNEFLQPF |
| 5983 | TNNEFVQPF |
| 5984 | TNNEIIQPF |
| 5985 | TNNEVIQPF |
| 5986 | TNNQFIQPF |
| 5987 | TNQCLFKTM |
| 5988 | TNSEFIQPF |
| 5989 | TNTEFIQPF |
| 5990 | TNYEFIQPF |
| 5991 | TRAQIQDEW |
| 5992 | TSDVVPCHH |
| 5993 | TSMNELADE |
| 5994 | TSNEFIQPF |
| 5995 | TVPMDHCWP |
| 5996 | TVPMDHCWS |
| 5997 | TWFHVAEMF |
| 5998 | TWLPWINER |
| 5999 | TYAVLKRTM |
| 6000 | VATDSVNES |
| 6001 | VGEERVGEP |
| 6002 | VGGERVGEA |
| 6003 | VGVMLNAMQ |
| 6004 | VKKIKRCEG |
| 6005 | VMEPLVCDD |
| 6006 | VMYPWISCL |
| 6007 | VNDDDQCCM |
| 6008 | VNDHDQCCM |
| 6009 | VNDNDQCCM |
| 6010 | VNDYAQCCM |
| 6011 | VNDYDKCCM |
| 6012 | VNDYDQCCR |
| 6013 | VNDYDQCCV |
| 6014 | VNDYDQCFM |
| 6015 | VNDYDQCWM |
| 6016 | VNDYDQCYM |
| 6017 | VNDYEQCCM |
| 6018 | VNDYVQCCM |
| 6019 | VNGHKFNEE |
| 6020 | VNGYDQCCM |
| 6021 | VNHYDQCCM |
| 6022 | VNNYDQCCM |
| 6023 | VQKLFNSTV |
| 6024 | VQLLSLCPS |
| 6025 | VSDLMHNVH |
| 6026 | VSGQIDEMS |
| 6027 | VVIQPCDER |
| 6028 | VWAAMEYEE |
| 6029 | VWDCQWDDF |
| 6030 | VWEMPPIDM |
| 6031 | VWESSSIIS |
| 6032 | VWKKPPIDM |
| 6033 | VWKMLPIDM |
| 6034 | VWKMPPIDT |
| 6035 | VWKMPPIDV |
| 6036 | VWKMPPLDM |
| 6037 | VWKMPPMDM |
| 6038 | VWKMPPVDM |
| 6039 | VWKMPQIDM |
| 6040 | VWKMPSIDM |
| 6041 | VWKMSPIDM |
| 6042 | VWKQWCWEF |
| 6043 | VWKRPPIDM |
| 6044 | VWKTPPIDM |
| 6045 | VWKVPPIDM |
| 6046 | VWQMPPIDM |
| 6047 | VWTMPPIDM |
| 6048 | WAKCQFGAT |
| 6049 | WCRMDNKKS |
| 6050 | WCRWPFYSE |
| 6051 | WHCDAMECF |
| 6052 | WILIMASSW |
| 6053 | WILIMAYAW |
| 6054 | WILIMAYSL |
| 6055 | WILIMAYTW |
| 6056 | WILIMEYSW |
| 6057 | WILIMSYSW |
| 6058 | WILIMTYSW |
| 6059 | WILIMVYSW |
| 6060 | WILSMAYSW |
| 6061 | WIMIMAYSW |
| 6062 | WIPIMAYSW |
| 6063 | WIQIMAYSW |
| 6064 | WIRIMAYSW |
| 6065 | WIVIMAYSW |
| 6066 | WLIHLWTQF |
| 6067 | WLIPLWAQF |
| 6068 | WLIPLWPQF |
| 6069 | WLIPLWTEF |
| 6070 | WLIPMWTQF |
| 6071 | WLIPPWTQF |
| 6072 | WLIPVWTQF |
| 6073 | WLIRLWTQF |
| 6074 | WLLIMAYSW |
| 6075 | WLLPLWTQF |
| 6076 | WPSIWKNEQ |
| 6077 | WPSIWNNER |
| 6078 | WQMRMQNKF |
| 6079 | WSKCQFGAA |
| 6080 | WSKCQFGAM |
| 6081 | WSKCRFGAT |
| 6082 | WSKFQFGAT |
| 6083 | WSKWQFGAT |
| 6084 | WSLIMAYSW |
| 6085 | WSQCQFGAT |
| 6086 | WSRCQFGAT |
| 6087 | WTIACIQRG |
| 6088 | WTNDCIQRG |
| 6089 | WVDSMLHKW |
| 6090 | WVLIMAYSW |
| 6091 | YASYKAMED |
| 6092 | YCFDSEDMG |
| 6093 | YDIMFTHQM |
| 6094 | YDQMMGLER |
| 6095 | YFAADERWG |
| 6096 | YFQQKHNLF |
| 6097 | YFTADEQWG |
| 6098 | YGGCWPREC |
| 6099 | YISDSVSHG |
| 6100 | YKVRKFWDP |
| 6101 | YPNGMTFEP |
| 6102 | YQEHFHNLI |
| 6103 | YQENFHNWI |
| 6104 | YQNDMTFEP |
| 6105 | YRSHEKFQF |
| 6106 | YRSPEKFEF |
| 6107 | YRSPEQFQF |
| 6108 | YRTPEKFQF |
| 6109 | YSDEMDWSK |
| 6110 | YSNEMDWNK |
| 6111 | YSSSKAMED |
| 6112 | YVGDSVSHG |
| 6113 | YVSDCVSHG |
| 6114 | YVSDGVSHG |
| 6115 | YVSDSVNHG |
| 6116 | YVSDSVSHS |
| 6117 | YVSDSVSRG |

### Example 33

AAV5 Variants with Colon Tissue Tropism as Identified by Machine Learning

This example describes preferred properties of engineered AAV5 variants that display colon tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display colon tissue tropism.

Favored biophysical properties and favored amino acid residues at each position in the 581 to 589 region of an AAV5 VP capsid polypeptide, which are associated with a higher probability of colon tissue tropism are described below. Any of the below machine learning-derived positional amino acid preferences described in TABLE 50 can be present alone or in combination with each other or in combination with any of the amino acid preferences observed and described in EXAMPLE 14, TABLE 27. Listed below in TABLE 50 are a summary of positional features shared between the top important features for colon tropism extracted from the ML models.

TABLE 50
Machine Learning-Derived Colon Tissue Tropism Rules

High mol mass at Xaa1
Xaa1 is selected from Y or W
High solubility at Xaa1
Xaa1 is selected from W, F, I, or L
Low solubility at Xaa2
Xaa2 is selected from D
Low mutability at Xaa2
Xaa2 is selected from P or K
Medium mol mass at Xaa2
Xaa2 is selected from D, E, N, K, M, Q, I, or L
Low hydropathy at Xaa2
Xaa2 is selected from D, E, R, K, H, N, or Q
Low mutability at Xaa3
Xaa3 is selected from K, V, P, or C
High solubility at Xaa3
Xaa3 is selected from W, F, I, or L
High average flexibility at Xaa5
Xaa5 is selected from S, P, G, R, E, or D
High surface accessibility at Xaa5
Xaa5 is selected from D or N
Low hydropathy at Xaa6
Xaa6 is selected from R
Low mutability at Xaa6
Xaa6 is selected from Y, R, F, or L
Low solubility at Xaa6
Xaa6 is selected from R or Q
High surface accessibility at Xaa6
Xaa6 is selected from E, R, or K
High average flexibility at Xaa6
Xaa6 is selected from G or R
Low solubility at Xaa8
Xaa8 is selected from D

TABLE 51 below provides sequences of the 581 to 589 region for variant AAV VP1 capsid polypeptides that exhibited colon tissue tropism and comport to one or more of the rules provided in TABLE 50. The present disclosure, thus, provides for rAAVs composed of engineered AAV5 VP1 capsid polypeptides having a SEQ ID NO: 2, wherein the Xaa1 to Xaa9 region is any one of SEQ ID NO: 11118-SEQ ID NO: 12117, as disclosed in TABLE 51. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 51
Sequences of the 581 to 589 Region in AAV5 VP1
Capsid Polypeptide that Drive Colon
Tissue Tropism

| SEQ ID | 581-589 |
|---|---|
| NO | Sequence |
| 11118 | ACCKVRRDC |
| 11119 | ACPVEFVPW |
| 11120 | ACYIDAAPN |
| 11121 | ACYLRNEDR |
| 11122 | ADASRTELD |
| 11123 | ADASRTEPD |
| 11124 | ADASRTKRD |
| 11125 | ADERPNATD |
| 11126 | ADIMKEIIF |
| 11127 | ADIMKESIV |
| 11128 | ADIMKVSIF |
| 11129 | ADIMMESIF |
| 11130 | ADIMQESIF |
| 11131 | ADIMRESIF |
| 11132 | ADINRWSYV |
| 11133 | ADITKESIF |
| 11134 | ADLHNNHEV |
| 11135 | ADLNNNHED |
| 11136 | ADRDGPHWY |
| 11137 | ADRGGPHWD |
| 11138 | AECCLEPKH |
| 11139 | AEDGADMIT |
| 11140 | AEGCKFYNG |
| 11141 | AELMMGSII |
| 11142 | AEQQHRLIE |
| 11143 | AFCRGHYKA |
| 11144 | AFCRGHYPA |
| 11145 | AFCRGHYRA |
| 11146 | AFCRGLYQA |
| 11147 | AFCRGRYQA |
| 11148 | AFDTFYAFS |
| 11149 | AFHHDRKYY |
| 11150 | AFKEFSLCC |
| 11151 | AFLYAAGWC |
| 11152 | AFNLDRKYY |
| 11153 | AFNQDRKYY |
| 11154 | AFPQNEHQR |
| 11155 | AFVTFYAFR |
| 11156 | AGPVEFFPW |
| 11157 | AGRQEKPDG |
| 11158 | AGRQEKPYV |
| 11159 | AGWQKCYDS |
| 11160 | AGWQMCDDS |
| 11161 | AGWQTCYDS |
| 11162 | AHDLATEWE |
| 11163 | AIDYLIDIL |
| 11164 | AINHEKTDW |
| 11165 | AKDPKPIRK |
| 11166 | ALNHDRKYY |
| 11167 | ALPDTNHAD |
| 11168 | ALSNDNAIL |
| 11169 | ALSNHNAVL |
| 11170 | AMHMTRGKC |
| 11171 | AMIWPQVLD |
| 11172 | AMQKTRGKC |
| 11173 | ANCDMKDPW |
| 11174 | ANCDMTDRW |
| 11175 | ANCNKRHNW |
| 11176 | ANNHEKTDL |
| 11177 | ANNHEKTEW |
| 11178 | ANNHVKTDW |
| 11179 | APPPHEVKI |
| 11180 | APPPHEVKS |
| 11181 | APPPREVKF |
| 11182 | APPQMCDFI |
| 11183 | AQFKDRSSF |
| 11184 | ARPKNLPLS |
| 11185 | AVTNHCIDR |
| 11186 | AVWQMCYDS |
| 11187 | AWDGSIEFL |
| 11188 | AYDAPRWFM |
| 11189 | AYFQARLLL |
| 11190 | AYIMKESIF |
| 11191 | AYNHDRKYY |
| 11192 | CDKMCAPQA |
| 11193 | CDPHGMVGC |
| 11194 | CDRKGAGNC |
| 11195 | CDRMYAPQA |
| 11196 | CERKGAGHC |
| 11197 | CERKGAGNG |
| 11198 | CERKGSGNC |
| 11199 | CERRGAGNC |
| 11200 | CFCKCGMPS |
| 11201 | CFDCSRAHP |
| 11202 | CFDRGKAFW |
| 11203 | CGFPGFSPV |
| 11204 | CGRWNFQYL |
| 11205 | CGSSGHLIY |
| 11206 | CHPLTAPSV |
| 11207 | CIHTCESII |
| 11208 | CINPCESII |
| 11209 | CLLDGKSDL |
| 11210 | CMCPPRLEV |
| 11211 | CMKFAFWEC |
| 11212 | CMKIAFWGC |
| 11213 | CMKMAFWEC |
| 11214 | CMPTARICM |
| 11215 | CQIWAKNEY |
| 11216 | CQIWGKNEF |
| 11217 | CQLWAKNEF |
| 11218 | CQPHWEKGD |
| 11219 | CRKMMQRIS |
| 11220 | CSGSGHLIY |
| 11221 | CWDERNVFC |
| 11222 | CWFMGEGCH |
| 11223 | CWLIWKLDR |
| 11224 | CWVLWKLDR |
| 11225 | DCKCDMFNC |
| 11226 | DCPLSNSYE |
| 11227 | DDKIAVGWP |
| 11228 | DDKIAVRWH |
| 11229 | DDVDCCDRK |
| 11230 | DFIMKYHAM |
| 11231 | DFNHDRKYY |
| 11232 | DFPQNEHQR |
| 11233 | DFQEFSLCC |
| 11234 | DGFMDFKHH |
| 11235 | DGMRDFKPG |
| 11236 | DGVVPVTNL |
| 11237 | DGYMDFKHL |
| 11238 | DGYMDFKRH |
| 11239 | DHALATEWE |
| 11240 | DHDLATGWE |
| 11241 | DHDLTTEWE |
| 11242 | DHDLVTEWE |
| 11243 | DHVLATEWE |
| 11244 | DKDCDLTSR |
| 11245 | DKDGDRFYI |
| 11246 | DKDHSRIDG |
| 11247 | DKHDSRIDG |
| 11248 | DKHHGRIDG |
| 11249 | DKHHSRFDG |
| 11250 | DKHHSRIDC |
| 11251 | DKHHSRIDD |
| 11252 | DKHHSRIDR |
| 11253 | DKHHSRIDV |
| 11254 | DKHHSRIHG |
| 11255 | DKHHSRITG |
| 11256 | DKHHSRIYG |
| 11257 | DKHHSRMDG |
| 11258 | DKHHSRNDG |
| 11259 | DKHHSRVDG |
| 11260 | DKHLSRIDG |
| 11261 | DKHNSRIDG |
| 11262 | DKHPSRIDG |
| 11263 | DKHRSRIDG |
| 11264 | DKLHSRIDG |
| 11265 | DKNHSRIDG |
| 11266 | DKPHSRIDG |
| 11267 | DKYHSRIDG |
| 11268 | DMHHSRIDG |
| 11269 | DNGFKLQKS |
| 11270 | DNQCSDKGL |
| 11271 | DPPNDMGAV |
| 11272 | DQHHSRIDG |
| 11273 | DRDCNLTSR |
| 11274 | DRHHSRIDG |
| 11275 | DSRCPAAYF |
| 11276 | DTFDRIFCS |
| 11277 | DWDHMFEME |
| 11278 | DWSHIFGRG |
| 11279 | DYNWHACSI |
| 11280 | EAIHRQACT |
| 11281 | EDIMKESIF |
| 11282 | EDNKWHGWH |
| 11283 | EEAPGFFCF |
| 11284 | EEAPGFICY |
| 11285 | EEATGFICF |
| 11286 | EEDPGFICF |
| 11287 | EEGPGFICF |
| 11288 | EEGPGFIFF |
| 11289 | EETPGFICF |
| 11290 | EFKEFSLCC |
| 11291 | EFPIDRRFP |
| 11292 | EGDEHEFAG |
| 11293 | EGDEPEFAG |
| 11294 | EGDEQEFVG |
| 11295 | EGFPFEGIS |
| 11296 | EGFRFEGII |
| 11297 | EGHNYEAML |
| 11298 | EGIYFVKHD |
| 11299 | EGQNYEALL |
| 11300 | EGQNYEAVL |
| 11301 | EGQNYESML |
| 11302 | EGQNYETML |
| 11303 | EGRNYEAML |
| 11304 | EIAYDYFPM |
| 11305 | EIILKDMCH |
| 11306 | EIILKDMWN |
| 11307 | EIIVKDMWH |
| 11308 | EKAPGFICF |
| 11309 | EKCGPCARY |
| 11310 | EKCMCHDIS |
| 11311 | EKHDGLMRR |
| 11312 | EKPDGLMRQ |
| 11313 | EKPEPRYKE |
| 11314 | EKPQPWYKE |
| 11315 | EKPSITCRE |
| 11316 | EMCMDVPSV |
| 11317 | EQCLAAAGE |
| 11318 | EQCLAAAVE |
| 11319 | EQCLAAGAE |
| 11320 | EQCLAAPAE |
| 11321 | EQCLAASAE |
| 11322 | EQCVAAAAE |
| 11323 | EQDIYGAIV |
| 11324 | EQPVGQSHR |
| 11325 | EQTEGQSHR |
| 11326 | ESINRQACT |
| 11327 | EVAPGFICF |
| 11328 | EVEYDYFPM |
| 11329 | EVIYFVQHD |
| 11330 | EVLYFVKHD |
| 11331 | EVWCQVEPN |
| 11332 | EWACAHAMR |
| 11333 | EWDQPNYIE |
| 11334 | EWVCAHAMR |
| 11335 | EYFQARLWL |
| 11336 | EYRQPQSIA |
| 11337 | FACPVLWGQ |
| 11338 | FANPVLWGQ |
| 11339 | FASPILWGQ |
| 11340 | FASPVLWGR |
| 11341 | FCWRDNEPE |
| 11342 | FDGPSVRGE |
| 11343 | FDKMYAPQA |
| 11344 | FDMCIEYSN |
| 11345 | FDMWGVGIP |
| 11346 | FDNHANTWA |
| 11347 | FDSPVLWGQ |
| 11348 | FEQTRFPMQ |
| 11349 | FEREFHYYD |
| 11350 | FEREFKYYD |
| 11351 | FERPPKLGI |
| 11352 | FEWEFQYYD |
| 11353 | FHDNARLFL |
| 11354 | FHLREKDDC |
| 11355 | FHYDCQDKV |
| 11356 | FHYDCRDNV |
| 11357 | FIAHKGQQW |
| 11358 | FIEFHVILV |
| 11359 | FIGKGYAIF |
| 11360 | FIGMGYAIC |
| 11361 | FIVHKGRQW |
| 11362 | FIVLKGQQW |
| 11363 | FIVMGYAIF |
| 11364 | FIVNKGQQW |
| 11365 | FLERYQHMD |
| 11366 | FLFMEPRNG |
| 11367 | FLGCYCHYA |
| 11368 | FLGCYCLYA |
| 11369 | FLGCYCPDA |
| 11370 | FLGCYCPSA |
| 11371 | FLGCYCPYG |
| 11372 | FLGCYCPYS |
| 11373 | FLGCYCRYA |
| 11374 | FLGCYFPYA |
| 11375 | FLGCYGPYA |
| 11376 | FLGCYRPYA |
| 11377 | FLGFYCPYA |
| 11378 | FLGMGYAIF |
| 11379 | FLGNTWLGP |
| 11380 | FLGWYCPYA |
| 11381 | FLGYYCPYA |
| 11382 | FLRNAWLGP |
| 11383 | FLRNTWLDP |
| 11384 | FLRNTWLGA |
| 11385 | FLRNTWLGS |
| 11386 | FLRNTWLSP |
| 11387 | FLRNTWLVP |
| 11388 | FMCNIGCFT |
| 11389 | FMGCYCPYA |
| 11390 | FNVCTMWAQ |
| 11391 | FPDCKNKAF |
| 11392 | FPLRENDDC |
| 11393 | FPTCWRYAK |
| 11394 | FPTIGLMAC |
| 11395 | FPYCKNKPF |
| 11396 | FQRNTWLGP |
| 11397 | FRIRCRVNK |
| 11398 | FRIRRRVNK |
| 11399 | FRRNTWLGP |
| 11400 | FSEQHAVND |
| 11401 | FSEQHAVNT |
| 11402 | FSEQHVVNN |
| 11403 | FSERHAVNN |
| 11404 | FSGWFRVCD |
| 11405 | FSPVERHNG |
| 11406 | FTSPVLWGQ |
| 11407 | FVCMEPRNG |
| 11408 | FVFMDPRNG |
| 11409 | FVFVEPRNG |
| 11410 | FVKGEFHFH |
| 11411 | FVQPFLHDG |
| 11412 | FWCRPRLHY |
| 11413 | FWPGHNPHN |
| 11414 | FWSRMIPLV |
| 11415 | FWTGHNPHI |
| 11416 | FWTGHNPPN |
| 11417 | FWTGRNPHN |
| 11418 | FYPGERHNG |
| 11419 | GCDGYALAI |
| 11420 | GCDPYNMVD |
| 11421 | GDDDCCDRK |
| 11422 | GDHHGMVGC |
| 11423 | GDKIKFSDR |
| 11424 | GDKMGICHC |
| 11425 | GDKMGIHHC |
| 11426 | GDKMYAPQA |
| 11427 | GDPHGMGGC |
| 11428 | GDPHGMLGC |
| 11429 | GDPPGMVGC |
| 11430 | GDPQGMVGC |
| 11431 | GDVDCCDRT |
| 11432 | GDYHYRVNW |
| 11433 | GEAPGFICF |
| 11434 | GENKVNDPA |
| 11435 | GEYHCRVNW |
| 11436 | GEYKLNDPA |
| 11437 | GEYKVNDPP |
| 11438 | GEYRVNDPA |
| 11439 | GFCRGHYQA |
| 11440 | GFMCNLCGP |
| 11441 | GFQTKFHLC |
| 11442 | GGDGYAIAI |
| 11443 | GGRHGYLHD |
| 11444 | GGWQMCYDS |
| 11445 | GGYKVNDPA |
| 11446 | GIIHHCVSE |
| 11447 | GIIRHCFSE |
| 11448 | GIIRHCLSE |
| 11449 | GIIRHCVIE |
| 11450 | GIIRNCVSE |
| 11451 | GKHHSRIDG |
| 11452 | GLRDGNSIF |
| 11453 | GMIPYNWSW |
| 11454 | GMPATELWL |
| 11455 | GMRRDGLDV |
| 11456 | GMRWDRDQL |
| 11457 | GMSSHTPIY |
| 11458 | GNCNKRHNW |
| 11459 | GPKKGGQAM |
| 11460 | GPKQGGQSM |
| 11461 | GPVLPDVGG |
| 11462 | GPVMPDVGA |
| 11463 | GRSSHPPIY |
| 11464 | GSKSGGADD |
| 11465 | GSKYGGDDD |
| 11466 | GSNSGGDDD |
| 11467 | GVRRVKIEW |
| 11468 | GWIEDKREV |
| 11469 | GWSEEKREV |
| 11470 | HDDWKKQPN |
| 11471 | HGAERFDRF |
| 11472 | HGAERVDRV |
| 11473 | HGLVDFDPH |
| 11474 | HKHHSRIDG |
| 11475 | HNHTRKDPV |
| 11476 | HVANEEVGG |
| 11477 | HVGNEEVSG |
| 11478 | HWRVQLPWN |
| 11479 | ICHANNVRV |
| 11480 | ICKARIEKY |
| 11481 | ICKPRLEKY |
| 11482 | ICPANNVRG |
| 11483 | ICPANNVRL |
| 11484 | ICPVNNVRV |
| 11485 | ICRPRIEKY |
| 11486 | ICSANNVRV |
| 11487 | ICSIDNNNY |
| 11488 | ICSMDNKNY |
| 11489 | IDHDNWDMR |
| 11490 | IEKDEMPVC |
| 11491 | IERPPKLGT |
| 11492 | IFLNIFELR |
| 11493 | IFLNIFEML |
| 11494 | IGANGWQCC |
| 11495 | IGLVDFDPH |
| 11496 | IHAPKLLWL |
| 11497 | IHCPDSWSQ |
| 11498 | IHCPEAWSQ |
| 11499 | IHCPESWSL |
| 11500 | IHGPCTPWD |
| 11501 | IHHEGRSVC |
| 11502 | IHPPKLLWL |
| 11503 | IHSPKLLWL |
| 11504 | IHTNMRVIS |
| 11505 | IHTNMRVIV |
| 11506 | IHTPRLLWL |
| 11507 | IIKFEWQEV |
| 11508 | IIKFEWQKF |
| 11509 | IIKFEWQQV |
| 11510 | IIKFEWQRV |
| 11511 | IKDQNKEWN |
| 11512 | ILRNTWLGP |
| 11513 | IMNEAHYRF |
| 11514 | IMNKDHYRF |
| 11515 | IMNRAHYRF |
| 11516 | INGSNTLTN |
| 11517 | IPECWWRWH |
| 11518 | IPEFWLRWH |
| 11519 | IPEIWWRWH |
| 11520 | IPKDEMPVC |
| 11521 | IPPLKTEDN |
| 11522 | IPRNVYMCD |
| 11523 | IPRTVYMCD |
| 11524 | IQKDEMPFC |
| 11525 | IQKDEMPVW |
| 11526 | IQKYEMPVC |
| 11527 | IQRDEMPVC |
| 11528 | IRPNDSSFH |
| 11529 | IRPNEGSFH |
| 11530 | IRPNESSFP |
| 11531 | ISGKEMNST |
| 11532 | ISNEMSKIL |
| 11533 | IVADNTVCA |
| 11534 | IVDDHTVCA |
| 11535 | IVDTNTATK |
| 11536 | IVPRFLVEA |
| 11537 | IWPANNVRV |
| 11538 | KDDWAGYQM |
| 11539 | KDIREIWDI |
| 11540 | KDIRGIWDI |
| 11541 | KDIRVIWDI |
| 11542 | KDMRDIWDI |
| 11543 | KDSWAGYQM |
| 11544 | KDWMPSYAL |
| 11545 | KDYWAEYQM |
| 11546 | KDYWAGCQM |
| 11547 | KDYWAGYKM |
| 11548 | KDYWAGYRM |
| 11549 | KDYWAVYQM |
| 11550 | KEGCHGRMG |
| 11551 | KEGCHVRVG |
| 11552 | KELSCDQNW |
| 11553 | KFLLAFEVT |
| 11554 | KFLYAAGWC |
| 11555 | KGIVTDRIL |
| 11556 | KHENKKDVL |
| 11557 | KIMQFCWDF |
| 11558 | KKFLCWEWS |
| 11559 | KLCNPVVFV |
| 11560 | KMCAYQPDT |
| 11561 | KMCHDERQV |
| 11562 | KMCPDERKV |
| 11563 | KMCPDERQL |
| 11564 | KMCTDERQV |
| 11565 | KMFPDERQV |
| 11566 | KMGPDERQV |
| 11567 | KMLIDEPTL |
| 11568 | KMLIDQATL |
| 11569 | KMLMDEATL |
| 11570 | KMLNDEATL |
| 11571 | KMRIDEATL |
| 11572 | KMWPDERQV |
| 11573 | KNFEELDND |
| 11574 | KNFEKLDNE |
| 11575 | KNFEKLDYD |
| 11576 | KNFEKLNND |
| 11577 | KNKCIENRW |
| 11578 | KPCPVRPPH |
| 11579 | KPDDEFDCD |
| 11580 | KPVYLYCSD |
| 11581 | KQCLAAAAE |
| 11582 | KSEWQLMYG |
| 11583 | KTADEFDCD |
| 11584 | KWERYRDFI |
| 11585 | KYKREEYEK |
| 11586 | LCKLPEIRK |
| 11587 | LDAKMVQPS |
| 11588 | LDCDSVWSP |
| 11589 | LDLKMVQPS |
| 11590 | LEGEGMWAF |
| 11591 | LERKPRNDC |
| 11592 | LERKPRNYF |
| 11593 | LERKPRSYC |
| 11594 | LERQPRNYC |
| 11595 | LESEGMWSF |
| 11596 | LHHEGRSVC |
| 11597 | LKHPPQPKV |
| 11598 | LKQINRHPC |
| 11599 | LMDAIDSIW |
| 11600 | LMVAIDSIW |
| 11601 | LNYCDQYGD |
| 11602 | LNYCDQYVG |
| 11603 | LNYERRDNY |
| 11604 | LPAYTTPIV |
| 11605 | LPFGNKFPD |
| 11606 | LPGPVTVSW |
| 11607 | LPKPHFDKQ |
| 11608 | LPKPYFDKK |
| 11609 | LPLLKTEDN |
| 11610 | LPLTDAPMV |
| 11611 | LPPLETEDN |
| 11612 | LPPLKTEAN |
| 11613 | LPPLKTEDD |
| 11614 | LPPLKTEDT |
| 11615 | LPPLKTKDN |
| 11616 | LPPLKTVDN |
| 11617 | LPPLRTEDN |
| 11618 | LQRLGYSPD |
| 11619 | LQTYTTPIV |
| 11620 | LRIDPKDSF |
| 11621 | LSICHPVMP |
| 11622 | LSICKPVMP |
| 11623 | LWDHGASYK |
| 11624 | LWHHGASYM |
| 11625 | LYDQSRSQL |
| 11626 | LYPCKMPMN |
| 11627 | LYYPSRSQL |
| 11628 | LYYQSRSKL |
| 11629 | MCKPRIEKY |
| 11630 | MCLIDGKDW |
| 11631 | MCSIDNKNY |
| 11632 | MDKHGCCCL |
| 11633 | MDKLGCCCW |
| 11634 | MDMQGGKVC |
| 11635 | MDQHGCCCW |
| 11636 | MDWCVQQSS |
| 11637 | MDWCVQQST |
| 11638 | MEADHKSDP |
| 11639 | MKGCFVHHS |
| 11640 | MKGYFIHHS |
| 11641 | MKGYFVHHY |
| 11642 | MMPCMVNFG |
| 11643 | MNKCIENRG |
| 11644 | MNKWIENRW |
| 11645 | MQFVDRLAM |
| 11646 | MQFVDRWAL |
| 11647 | MRCCNVPWS |
| 11648 | MRDRMKDEE |
| 11649 | MVIHPAISR |
| 11650 | MWDHRNETM |
| 11651 | MWDNRNESM |
| 11652 | NAMKRSPDV |
| 11653 | NAMQRSPDF |
| 11654 | NCINATDGD |
| 11655 | NCLVDFDPH |
| 11656 | NCMNATDWD |
| 11657 | NCNNELTDR |
| 11658 | NDMAQSYRE |
| 11659 | NDMEQSDRE |
| 11660 | NDMEQSYHE |
| 11661 | NELAYQDWS |
| 11662 | NELEYQHWS |
| 11663 | NFCPENAWQ |
| 11664 | NFEDQESHV |
| 11665 | NFGDLESHV |
| 11666 | NFITQEMYS |
| 11667 | NFNTQEMYS |
| 11668 | NFTTKEMYS |
| 11669 | NFTTMNMTC |
| 11670 | NFTTQEMYY |
| 11671 | NFWMDMHRI |
| 11672 | NGGTWANVE |
| 11673 | NGHNELTDR |
| 11674 | NGLDDFDPH |
| 11675 | NGLVDCDPH |
| 11676 | NGLVDFAPH |
| 11677 | NGLVDFDPL |
| 11678 | NGLVDFDRH |
| 11679 | NGLVDFDTH |
| 11680 | NGLVDFVPH |
| 11681 | NGLVHFDPH |
| 11682 | NGNNELADR |
| 11683 | NGNNELTDK |
| 11684 | NGNNGLTDR |
| 11685 | NGRVDFDPH |
| 11686 | NGWEGSEDD |
| 11687 | NGWSDVDRQ |
| 11688 | NHNVPACII |
| 11689 | NIEQMLAIG |
| 11690 | NKHHSRIDG |
| 11691 | NMHSEQTPL |
| 11692 | NMKFSVGMN |
| 11693 | NMKISVDMN |
| 11694 | NMKTESAHK |
| 11695 | NMKTKSADK |
| 11696 | NMKTKSAHE |
| 11697 | NMKTKSAHT |
| 11698 | NMKTKSAPK |
| 11699 | NMKTKSARK |
| 11700 | NMKTKSDHK |
| 11701 | NMKTKTAHK |
| 11702 | NMKTRSAHK |
| 11703 | NMPWNMEWN |
| 11704 | NMPWNMPWN |
| 11705 | NMPWNMQLN |
| 11706 | NMPWNMQWK |
| 11707 | NMPWNMQWS |
| 11708 | NMPWNVQWN |
| 11709 | NMQTKSAHK |
| 11710 | NNHMVAKIA |
| 11711 | NPCHGYLKM |
| 11712 | NPPCARECR |
| 11713 | NPPCARGGR |
| 11714 | NPPCARKWR |
| 11715 | NPPCARQWR |
| 11716 | NPRCGIFTL |
| 11717 | NQGLPCCQI |
| 11718 | NQGMHCCQI |
| 11719 | NQGMPCCRI |
| 11720 | NQNNLEYPI |
| 11721 | NRHPPAAIS |
| 11722 | NRNPPAAII |
| 11723 | NRNPPEAIS |
| 11724 | NRNSFHQFE |
| 11725 | NRPWQMRWA |
| 11726 | NRTSFHKFE |
| 11727 | NSITANCSK |
| 11728 | NSITENCAK |
| 11729 | NSITENCSN |
| 11730 | NTKVKDEDN |
| 11731 | NVLGPPSFN |
| 11732 | NVNNELTDR |
| 11733 | NVTANYCCF |
| 11734 | NYDHFIAAS |
| 11735 | NYGKDMHKL |
| 11736 | NYNNFIAAS |
| 11737 | NYRESRSDL |
| 11738 | PAIHNHMCT |
| 11739 | PAIRNHMCP |
| 11740 | PAYKNSGGD |
| 11741 | PDFVAEQQP |
| 11742 | PETLWANIW |
| 11743 | PETLWTDIW |
| 11744 | PETPWTNIW |
| 11745 | PFCRGHYQA |
| 11746 | PFISPKQVS |
| 11747 | PFISPREVS |
| 11748 | PFIYTDLGP |
| 11749 | PFLYTDWGP |
| 11750 | PFNNWFAAG |
| 11751 | PGWQMCYDS |
| 11752 | PINFPYEWL |
| 11753 | PKFAFNQCY |
| 11754 | PKGMINWPS |
| 11755 | PKVRINWPS |
| 11756 | PKVVINWPS |
| 11757 | PMCMEPFPM |
| 11758 | PMNRDTLIS |
| 11759 | PPPLNRKWD |
| 11760 | PQAYTTPIV |
| 11761 | PQLYFTTDK |
| 11762 | PQTKKDCPA |
| 11763 | PRKMMQRIS |
| 11764 | PSDLKLVNN |
| 11765 | PSTIKHVDP |
| 11766 | PSTIKRIDP |
| 11767 | PWDGSIEFL |
| 11768 | QCLLWNDCK |
| 11769 | QELFHGKIA |
| 11770 | QFPRHEPIQ |
| 11771 | QFPRQEPLQ |
| 11772 | QFYNRSDDL |
| 11773 | QIDQFCPIV |
| 11774 | QINPFYMWF |
| 11775 | QINQFCQIV |
| 11776 | QMCPDERQV |
| 11777 | QPCLVRPPH |
| 11778 | QPCTVRPPH |
| 11779 | QSMWETDRN |
| 11780 | QWCWMAVSI |
| 11781 | QWNMFQVIF |
| 11782 | RAAKVIPPT |
| 11783 | RADKFIPPT |
| 11784 | RADKVITPT |
| 11785 | RAYKVIPPT |
| 11786 | RCDLKEMWK |
| 11787 | RCYLKEMRK |
| 11788 | RDCHMRITD |
| 11789 | RDCWMVFII |
| 11790 | RDFVAGQQP |
| 11791 | RDFVGEQQP |
| 11792 | RDGKWALEN |
| 11793 | RDGKWVREN |
| 11794 | RDYHMLITD |
| 11795 | RGFHHADNV |
| 11796 | RGFNHADNG |
| 11797 | RHIYRKGWG |
| 11798 | RLRDGNSIF |
| 11799 | RMCPYEAWK |
| 11800 | RMCTYEPWK |
| 11801 | RPEFNAFQA |
| 11802 | RSFIDIVGT |
| 11803 | RTQPIGVDP |
| 11804 | RYCWMVVII |
| 11805 | SADYNFGFS |
| 11806 | SCGWDHGNL |
| 11807 | SCPANNVRV |
| 11808 | SCPVEFFPW |
| 11809 | SDASRTERD |
| 11810 | SDKMGIYHC |
| 11811 | SDLHNNHED |
| 11812 | SDNWGAQCC |
| 11813 | SDPHGMVGC |
| 11814 | SFGPSQVCC |
| 11815 | SFIGNAQWC |
| 11816 | SFIGNARWC |
| 11817 | SFLAHNNFH |
| 11818 | SFLANNHFH |
| 11819 | SFLANNTFH |
| 11820 | SFNHDRKYY |
| 11821 | SFYEWPHCE |
| 11822 | SGICCCDNA |
| 11823 | SGIGCCDNS |
| 11824 | SGWKMCYDS |
| 11825 | SGWQMCYDS |
| 11826 | SHDQALMFE |
| 11827 | SHFYEDARS |
| 11828 | SIIRHCVSE |
| 11829 | SIQPFLHDG |
| 11830 | SLNGYTLVC |
| 11831 | SLPEYSNVK |
| 11832 | SNPWPMGKV |
| 11833 | SPHHDKDPF |
| 11834 | SPMINLWPS |
| 11835 | SRKMMKRIS |
| 11836 | SRKMMPRIS |
| 11837 | SRQCPLEIG |
| 11838 | SSDLKLVNS |
| 11839 | SSGGDHGNL |
| 11840 | SSIHHQCCT |
| 11841 | SSIQHHCCT |
| 11842 | SSVWDHGNL |
| 11843 | SVQLFLHDG |
| 11844 | SVQPFLHDA |
| 11845 | SVQPFLYDG |
| 11846 | SVQPVLHDG |
| 11847 | SYDNWDFCC |
| 11848 | TCSIDNKNY |
| 11849 | TCTWIGAGS |
| 11850 | TDASRTERD |
| 11851 | TDIMKESIF |
| 11852 | TDYWAGYQM |
| 11853 | TFPDGEMYQ |
| 11854 | TGLVDFDPH |
| 11855 | TGTWIGTGS |
| 11856 | TGWQMCYDS |
| 11857 | TIDDHSFDQ |
| 11858 | TINCKWCFC |
| 11859 | TINPHKSDW |
| 11860 | TISCKWCVC |
| 11861 | TNICYDQIK |
| 11862 | TNICYDQIR |
| 11863 | TNNHEKTDW |
| 11864 | TPCCLHHPA |
| 11865 | TPPPHEVKF |
| 11866 | TQMFAMPPS |
| 11867 | TQPPLMNIL |
| 11868 | TRNPPAAIS |
| 11869 | TRPSAASIR |
| 11870 | TRPSAASVG |
| 11871 | TRPSASSIG |
| 11872 | TSICYDQIQ |
| 11873 | TSPPDQVPV |
| 11874 | TWDGSIEFL |
| 11875 | VADHRPMYS |
| 11876 | VAIVDGWIE |
| 11877 | VAPSESSWE |
| 11878 | VCLVDGKDW |
| 11879 | VCPANNVRV |
| 11880 | VCTIYYMCG |
| 11881 | VDCNKRHNW |
| 11882 | VDHCTAIGA |
| 11883 | VDIFDGWIE |
| 11884 | VDPFCAVRI |
| 11885 | VDPMGEAGT |
| 11886 | VDVDCCDRK |
| 11887 | VEFADNPWN |
| 11888 | VERGNKIAS |
| 11889 | VERSNKISS |
| 11890 | VFCRGHYQA |
| 11891 | VGTKEGPDP |
| 11892 | VHHEGRSFC |
| 11893 | VHHEGRSVF |
| 11894 | VHHKGRSVC |
| 11895 | VHLRENDDC |
| 11896 | VHPEGRSVC |
| 11897 | VHQEGRSVC |
| 11898 | VHREGRSVC |
| 11899 | VHYEGRSVC |
| 11900 | VISRECYEE |
| 11901 | VKCKDKYQS |
| 11902 | VKCQDKYQY |
| 11903 | VKHHSRIDG |
| 11904 | VLGLPPYPE |
| 11905 | VLLYMRNNQ |
| 11906 | VMAAEQLNQ |
| 11907 | VMIWPQILD |
| 11908 | VMPTDCILV |
| 11909 | VNCDKRHNW |
| 11910 | VNCNKRHNL |
| 11911 | VNCNKRHNM |
| 11912 | VNCNKRRNW |
| 11913 | VNCNKRYNW |
| 11914 | VNCNNRHNC |
| 11915 | VNCNRRHNW |
| 11916 | VNCSKRHNW |
| 11917 | VNCYKRHNW |
| 11918 | VNGNKRHNW |
| 11919 | VNLQDLSDR |
| 11920 | VNPSDQTNH |
| 11921 | VNSSDQNNH |
| 11922 | VPDFDPFWS |
| 11923 | VPIFDGWIE |
| 11924 | VPNFAAFCM |
| 11925 | VPPACRHKK |
| 11926 | VPPGCRHQK |
| 11927 | VPTFDPFWS |
| 11928 | VQFVDRWAM |
| 11929 | VQTKKDCPA |
| 11930 | VRFDMCKWT |
| 11931 | VRFEMMSWT |
| 11932 | VTDFHTDML |
| 11933 | VTDFNTDMQ |
| 11934 | VVFINYNDQ |
| 11935 | VVFTNYNDK |
| 11936 | VVPPGNVIL |
| 11937 | VVPPGNVLP |
| 11938 | VVYRDTCNG |
| 11939 | VWDHRNESM |
| 11940 | VYNWHACAI |
| 11941 | WAEDAYRRF |
| 11942 | WCACVFEYW |
| 11943 | WCERVFEYW |
| 11944 | WCEWVFEYW |
| 11945 | WCFKKELCN |
| 11946 | WCKLPEIRR |
| 11947 | WCQCVFEYW |
| 11948 | WCQLPEIRK |
| 11949 | WCYLMNLES |
| 11950 | WDHNCQATI |
| 11951 | WDHNYQDTI |
| 11952 | WDNNCQDTI |
| 11953 | WDQNCQDTI |
| 11954 | WERMGQIEC |
| 11955 | WERMGQIED |
| 11956 | WERMGQIEH |
| 11957 | WERMGQIES |
| 11958 | WERMGQIQY |
| 11959 | WERMGQMEY |
| 11960 | WEWDRPFMK |
| 11961 | WFFHYKPWM |
| 11962 | WFMKCNWWT |
| 11963 | WGCCEEMDS |
| 11964 | WINTGPLRH |
| 11965 | WKIKPDHRV |
| 11966 | WKLKNCKSD |
| 11967 | WLFEGKLDT |
| 11968 | WLFYGCEPN |
| 11969 | WLPHGPRME |
| 11970 | WLPNRRIDD |
| 11971 | WLQDGNSIF |
| 11972 | WLRDGDSIF |
| 11973 | WLRDGNSIS |
| 11974 | WLRDGNSIY |
| 11975 | WLREGNSIF |
| 11976 | WLRGGNSIF |
| 11977 | WLRVGNSIF |
| 11978 | WMGWDRDQL |
| 11979 | WMPNRRIDA |
| 11980 | WMPNRRIDG |
| 11981 | WMRCDRDQL |
| 11982 | WMRLDRDQL |
| 11983 | WMRWARDQL |
| 11984 | WMRWDQDQL |
| 11985 | WMRWDRAQL |
| 11986 | WMRWDREQL |
| 11987 | WMSDMLKAV |
| 11988 | WMWWDRDQL |
| 11989 | WNIPNRSQA |
| 11990 | WNNEHLKVW |
| 11991 | WNNFYCGCN |
| 11992 | WPFPVQFCD |
| 11993 | WPHHAKDPF |
| 11994 | WPHHDKDPV |
| 11995 | WPHHNKDPF |
| 11996 | WPRDGNSIF |
| 11997 | WPSEGLIGA |
| 11998 | WPVPVQVCD |
| 11999 | WQIWAKNEF |
| 12000 | WRFDGCEPN |
| 12001 | WRFYGCEPT |
| 12002 | WRFYGCERN |
| 12003 | WRFYGCESN |
| 12004 | WRGCMFMSK |
| 12005 | WRNCMFMSK |
| 12006 | WRSYGCEPN |
| 12007 | WSSEGLIGA |
| 12008 | WVWNGCRDM |
| 12009 | WYAQCWKAF |
| 12010 | WYCWMVFII |
| 12011 | WYDAGHKCK |
| 12012 | WYDAGNNCK |
| 12013 | WYDAVNKCK |
| 12014 | WYDDGNKCK |
| 12015 | WYECVFEYW |
| 12016 | WYYAGNKCK |
| 12017 | YAHYCWDPI |
| 12018 | YAVAVGISS |
| 12019 | YDFGGRVQT |
| 12020 | YDHHKIIWA |
| 12021 | YDHHKIIWV |
| 12022 | YDINYYPEE |
| 12023 | YDINYYPKD |
| 12024 | YDINYYPNE |
| 12025 | YDIPKSTCS |
| 12026 | YDNSKYCKN |
| 12027 | YDTNYYPKE |
| 12028 | YDYGGRVQK |
| 12029 | YEAKSLQPN |
| 12030 | YEENVLFMG |
| 12031 | YEINYYPKE |
| 12032 | YETGMWLNR |
| 12033 | YFDCSRAHS |
| 12034 | YFDCSRANP |
| 12035 | YFDCSRGHP |
| 12036 | YFDCSRTHP |
| 12037 | YFDRSRAHP |
| 12038 | YFDWSRAHP |
| 12039 | YGCNRSYQD |
| 12040 | YHANNMEPW |
| 12041 | YHDLATEWE |
| 12042 | YHDNALLFL |
| 12043 | YHENVLFMG |
| 12044 | YHINNMEPW |
| 12045 | YHPHHMYWR |
| 12046 | YHPRTALMN |
| 12047 | YHQHNACMR |
| 12048 | YHTIDMEPW |
| 12049 | YHTINMEPW |
| 12050 | YHTNNMEPL |
| 12051 | YHTNNMERW |
| 12052 | YHTNNMETW |
| 12053 | YHTNNMVPW |
| 12054 | YHTNNREPW |
| 12055 | YHTNNVEPW |
| 12056 | YHTSNMEPW |
| 12057 | YHTTNMEPW |
| 12058 | YHTYNMEPW |
| 12059 | YIVHKGQQW |
| 12060 | YIWLVMFFS |
| 12061 | YKAGDRFYI |
| 12062 | YKDCDRFYI |
| 12063 | YKDDDRFYI |
| 12064 | YKDGDRIYI |
| 12065 | YKDSDRFYI |
| 12066 | YKHHSRIDG |
| 12067 | YKYGDRFYI |
| 12068 | YLPSAELIK |
| 12069 | YMWICPPDQ |
| 12070 | YMWIHPPDQ |
| 12071 | YMWISPPDQ |
| 12072 | YMWIYAPDQ |
| 12073 | YMWIYPPDK |
| 12074 | YMWIYPPDL |
| 12075 | YMWIYPPDP |
| 12076 | YMWIYPPGQ |
| 12077 | YMWIYPTDQ |
| 12078 | YMWIYQPDQ |
| 12079 | YMWNYPPDQ |
| 12080 | YMWSYPPDQ |
| 12081 | YMWTYPPDQ |
| 12082 | YNMAHGDEP |
| 12083 | YNMVDGDEP |
| 12084 | YNMVHGDEA |
| 12085 | YNMVHGDES |
| 12086 | YNMVHGDET |
| 12087 | YNMVHGDGP |
| 12088 | YNMVHSDEP |
| 12089 | YNPRIALMN |
| 12090 | YNPRSALMN |
| 12091 | YNTVHGDEP |
| 12092 | YNVCTLWAQ |
| 12093 | YNVCTMLAQ |
| 12094 | YNVVHGDEP |
| 12095 | YPEDRLNMR |
| 12096 | YPLGSAGSD |
| 12097 | YPQHNACMQ |
| 12098 | YPTNNMEPW |
| 12099 | YQAAARQNA |
| 12100 | YQADPRQNA |
| 12101 | YQATPRQNA |
| 12102 | YQAYGRGHV |
| 12103 | YQDAPRQNA |
| 12104 | YQIFQYPLH |
| 12105 | YQLFQYPWH |
| 12106 | YQLLHTIEN |
| 12107 | YQLLHTIEQ |
| 12108 | YRTLQCKWE |
| 12109 | YRWIYPPDQ |
| 12110 | YRYLWPIVR |
| 12111 | YTGLNELQN |
| 12112 | YTICDVPDP |
| 12113 | YTPNFRIGD |
| 12114 | YVIPFEMDD |
| 12115 | YVIPVEMDN |
| 12116 | YYDCSRAHP |
| 12117 | YYPCVQYRQ |

### Example 34

AAV5 Variants with Heart Tissue Tropism as Identified by Machine Learning

This example describes preferred properties of engineered AAV5 variants that display heart tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display heart tissue tropism.

Favored biophysical properties and favored amino acid residues at each position in the 581 to 589 region of an AAV5 VP capsid polypeptide, which are associated with a higher probability of heart tissue tropism are described below. Any of the below machine learning-derived positional amino acid preferences described in TABLE 52 can be present alone or in combination with each other or in combination with any of the amino acid preferences observed and described in EXAMPLE 13, TABLE 25. Listed below in TABLE 52 are a summary of positional features shared between the top important features for heart tropism extracted from the ML models.

TABLE 52
Machine Learning-Derived Heart Tissue Tropism Rules

Low solubility at Xaa1
Xaa1 is selected from N or E
Low hydropathy at Xaa1
Xaa1 is selected from H, N, Q, P, Y, D, or E
High mutability at Xaa1
Xaa1 is selected from A or E
High hydropathy at Xaa2
Xaa2 is selected from V or I
Medium mutability at Xaa2
Xaa2 is selected from V
Medium volume at Xaa2
Xaa2 is selected from V, E, or Q
High solubility at Xaa2
Xaa2 is selected from V or M
Low solubility at Xaa3
Xaa3 is selected from R or Q
Low surface accessibility at Xaa4
Xaa4 is selected from C
High solubility at Xaa4
Xaa4 is selected from C
Low charge at Xaa4
Xaa4 is selected from D, E, Y, W, V, P, M,
A, G, F, I, L, N, Q, S, T, or C
High hydropathy at Xaa4
Xaa4 is selected from C
High surface accessibility at Xaa5
Xaa5 is selected from D, E, R, K, N, or Q
Low solubility at Xaa5
Xaa5 is selected from D
Low mutability at Xaa6
Xaa6 is selected from C
Low solubility at Xaa6
Xaa6 is selected from D
High surface accessibility at Xaa8
Xaa8 is selected from D or N
High average flexibility at Xaa8
Xaa8 is selected from D, R, P, G, or S
Medium mol mass at Xaa9
Xaa9 is selected from N, D, L, or I

TABLE 53 below provides sequences of the 581 to 589 region for variant AAV VP1 capsid polypeptides that exhibited heart tissue tropism and comport to one or more of the rules provided in TABLE 52. The present disclosure, thus, provides for rAAVs composed of engineered AAV5 VP1 capsid polypeptides having a SEQ ID NO: 2, wherein the Xaa1 to Xaa9 region is any one of SEQ ID NO: 14118-SEQ ID NO: 15117, as disclosed in TABLE 53. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 53
Sequences of the 581 to 589 Region in AAV5 VP1
Capsid Polypeptide that Drive Heart
Tissue Tropism

| SEQ ID | 581-589 |
|---|---|
| NO | Sequence |
| 14118 | AADFFAIND |
| 14119 | AADIFAISD |
| 14120 | AADIFGIND |
| 14121 | AADIFVIND |
| 14122 | AADIYAIND |
| 14123 | AADMFAIND |
| 14124 | AAYIFAIND |
| 14125 | ACDCHHWDQ |
| 14126 | ACIEWNWGE |
| 14127 | AGDCCLAER |
| 14128 | AHECCLPNR |
| 14129 | AIQPRDMYS |
| 14130 | AKECTGLKN |
| 14131 | AKGMEKLRT |
| 14132 | AKNGDVLEM |
| 14133 | ALPCDNWRG |
| 14134 | APRCESCNM |
| 14135 | AQIDCITGL |
| 14136 | ASDYCTTER |
| 14137 | ATMNLSWAL |
| 14138 | AVCDDHKPN |
| 14139 | AVDWHCEVL |
| 14140 | AVESKDIPQ |
| 14141 | AVGDEQPTD |
| 14142 | CCECGHWPN |
| 14143 | CDDCYLINR |
| 14144 | CDICKHEEY |
| 14145 | CDNCYAEPH |
| 14146 | CDWMASVSV |
| 14147 | CDWMPNVSV |
| 14148 | CDWMSNVSV |
| 14149 | CEYGWILKR |
| 14150 | CGDWDFGEW |
| 14151 | CKCCRMEKC |
| 14152 | CKFRDFAES |
| 14153 | CKMLCRPPV |
| 14154 | CKPGDFVRK |
| 14155 | CLECCTDSA |
| 14156 | CPDCGPTGP |
| 14157 | CPECSVDPW |
| 14158 | CQMLCRSPM |
| 14159 | CRDCCLRGW |
| 14160 | CRECSNERV |
| 14161 | CRECVHNRC |
| 14162 | CRICRATIW |
| 14163 | CSDPKNMSI |
| 14164 | CWECCNDSG |
| 14165 | CWECCPDSG |
| 14166 | CWECCTDGG |
| 14167 | CWECCTDNG |
| 14168 | CWECCTDRV |
| 14169 | CWECCTDSV |
| 14170 | CWECCTDSW |
| 14171 | CWECFTDSG |
| 14172 | CWECGTDSG |
| 14173 | CWECYTDSG |
| 14174 | CWEWCTDSG |
| 14175 | CWGCCTDSG |
| 14176 | CWVCCTDSG |
| 14177 | CYWCGWDNR |
| 14178 | DAMDQREQY |
| 14179 | DAVDIVKDE |
| 14180 | DCNQFNWSE |
| 14181 | DDDDFAEKG |
| 14182 | DDEILKEDG |
| 14183 | DDIVGKPKW |
| 14184 | DDVEKDETN |
| 14185 | DERERKTKD |
| 14186 | DFKALDVPC |
| 14187 | DFKVLDVQC |
| 14188 | DGCDDHKPN |
| 14189 | DGKIEPIRF |
| 14190 | DGMRCKEKK |
| 14191 | DGVGRPGDN |
| 14192 | DHRIDNIDA |
| 14193 | DIDDLNFAT |
| 14194 | DIDFMECAF |
| 14195 | DIEEWEMAE |
| 14196 | DIEGQNNGM |
| 14197 | DIEHIRDPP |
| 14198 | DIEMFNENG |
| 14199 | DIEMNITAL |
| 14200 | DIGDIWMSQ |
| 14201 | DIIEYQKVE |
| 14202 | DIIPKDAAP |
| 14203 | DIKVLDSQP |
| 14204 | DILCICETS |
| 14205 | DINNNNCGY |
| 14206 | DIQPFDYYL |
| 14207 | DIQPNDGCS |
| 14208 | DIRDEDNIG |
| 14209 | DIRDSWTES |
| 14210 | DITEEDVTL |
| 14211 | DITHHNDGK |
| 14212 | DIVDHDHAG |
| 14213 | DIVECIQYN |
| 14214 | DIYNDSWST |
| 14215 | DLCRPNPKN |
| 14216 | DLIVATELF |
| 14217 | DLIVVTEWF |
| 14218 | DLPNSQPPH |
| 14219 | DLSEPTEWF |
| 14220 | DLTVATEWF |
| 14221 | DMDCPEDQQ |
| 14222 | DMFQLKPQN |
| 14223 | DMVDLDVNP |
| 14224 | DNVNMNDAI |
| 14225 | DQRCNKAPG |
| 14226 | DRCDSIILA |
| 14227 | DSDAKNMSI |
| 14228 | DSDPKNMS |
| 14229 | DSDSKNMSI |
| 14230 | DSDTKNMSI |
| 14231 | DSNDQDTPE |
| 14232 | DSSEVDMNF |
| 14233 | DSYDDSWST |
| 14234 | DSYNDGWST |
| 14235 | DSYNDSWSP |
| 14236 | DTAPKQEAE |
| 14237 | DTIQKKMDH |
| 14238 | DTIWADINN |
| 14239 | DVADWNGYD |
| 14240 | DVAERVEQI |
| 14241 | DVAGLDKNA |
| 14242 | DVAPMDQVE |
| 14243 | DVAPMHEVE |
| 14244 | DVAPMHQDE |
| 14245 | DVAPMHQIE |
| 14246 | DVAPMHRVE |
| 14247 | DVAPMNQVE |
| 14248 | DVCDDHKAN |
| 14249 | DVCDDHKPH |
| 14250 | DVCDDHKPI |
| 14251 | DVCDDHKPS |
| 14252 | DVCDDHKPT |
| 14253 | DVCDDHKQN |
| 14254 | DVCDDHKRN |
| 14255 | DVCDDHKSN |
| 14256 | DVCDDHKTN |
| 14257 | DVCDDHQPN |
| 14258 | DVCDDHTPN |
| 14259 | DVCDDPKPN |
| 14260 | DVCPFPDGK |
| 14261 | DVCPVDVQN |
| 14262 | DVDDIHVSF |
| 14263 | DVDEISDGV |
| 14264 | DVDIHEWPP |
| 14265 | DVDMIDTCP |
| 14266 | DVDPWDVTE |
| 14267 | DVDQMHHND |
| 14268 | DVDTVEMES |
| 14269 | DVEEKDNKA |
| 14270 | DVEETEAAY |
| 14271 | DVEHCNTNA |
| 14272 | DVEHQDWGG |
| 14273 | DVEIHKWGG |
| 14274 | DVETTDNLK |
| 14275 | DVEWDNMKQ |
| 14276 | DVGDDHKPN |
| 14277 | DVGEDEFQP |
| 14278 | DVGEWTMNN |
| 14279 | DVGILDFNG |
| 14280 | DVGPMDCQP |
| 14281 | DVHEYDHPE |
| 14282 | DVHFFENSR |
| 14283 | DVIPRDGAE |
| 14284 | DVKDQSIML |
| 14285 | DVKEIDELC |
| 14286 | DVMPMPEAQ |
| 14287 | DVNEDGFNM |
| 14288 | DVNENEWTD |
| 14289 | DVRDSMKRD |
| 14290 | DVSDFIQHF |
| 14291 | DVSEGDDST |
| 14292 | DVSQIPITF |
| 14293 | DVTEYQWST |
| 14294 | DVTPNSWIE |
| 14295 | DVTPTSWSE |
| 14296 | DVTTLNESQ |
| 14297 | DVVDFSIVK |
| 14298 | DVVEQRECP |
| 14299 | DVYDDHKPN |
| 14300 | DVYEKMWAN |
| 14301 | DYFDMNLTD |
| 14302 | DYGPEDFSM |
| 14303 | EAAPRDSDD |
| 14304 | EAECKMWPT |
| 14305 | EAELGNVNM |
| 14306 | EAGCRMWPT |
| 14307 | EARIQCKID |
| 14308 | EARMDIWPI |
| 14309 | EAVNRPNMK |
| 14310 | ECGCMYWCY |
| 14311 | ECGCMYWGC |
| 14312 | ECGCMYWVY |
| 14313 | EDDWQRMLY |
| 14314 | EDRIQCKID |
| 14315 | EDRMDKIFT |
| 14316 | EDTGEREAW |
| 14317 | EDYAACFEF |
| 14318 | EEGMQMISQ |
| 14319 | EERCYDGSW |
| 14320 | EFHEFRASN |
| 14321 | EFRIQCKID |
| 14322 | EIAIYNFAN |
| 14323 | EIAPNMCPP |
| 14324 | EIAPREVVN |
| 14325 | EICPVDRTQ |
| 14326 | EICSFEEGH |
| 14327 | EIDDDMLEE |
| 14328 | EIDDTDQRW |
| 14329 | EIDDVGEPG |
| 14330 | EIDEFSASN |
| 14331 | EIDQKDISQ |
| 14332 | EIDRGDSYK |
| 14333 | EIEALMKER |
| 14334 | EIEEDDEWY |
| 14335 | EIEEFMKMR |
| 14336 | EIEQDNEPG |
| 14337 | EIEQSFLDS |
| 14338 | EIEVTAWAC |
| 14339 | EIEYIDAGG |
| 14340 | EIGCVDSAK |
| 14341 | EIGMQIAQE |
| 14342 | EIGTFDNSE |
| 14343 | EIHDQQIVF |
| 14344 | EIHDVDFGM |
| 14345 | EIHEFFHNR |
| 14346 | EIHEFGASN |
| 14347 | EIHEFPASN |
| 14348 | EIHEFRAAN |
| 14349 | EIHEFRAPN |
| 14350 | EIHEFRASI |
| 14351 | EIHEFRAST |
| 14352 | EIHEFRAYN |
| 14353 | EIHEFRDSN |
| 14354 | EIHEFRGSN |
| 14355 | EIHEFRSSN |
| 14356 | EIHEFSASN |
| 14357 | EIHEISTHD |
| 14358 | EIHGMDTCP |
| 14359 | EIHMFEQSE |
| 14360 | EIHPWHRTN |
| 14361 | EIIDTSCNC |
| 14362 | EIISMNDFM |
| 14363 | EILDYCSYN |
| 14364 | EILVRCEVD |
| 14365 | EIMPDSIMC |
| 14366 | EIMPNHNMI |
| 14367 | EINDQRPWP |
| 14368 | EINEFRASN |
| 14369 | EINPKSEDE |
| 14370 | EIPEFRASN |
| 14371 | EIQDIVKID |
| 14372 | EIQPLNAVN |
| 14373 | EIRCHHKFA |
| 14374 | EIRDMAHHG |
| 14375 | EIRDTHEMQ |
| 14376 | EIREFRASN |
| 14377 | EIREQCIGP |
| 14378 | EIRFEGNME |
| 14379 | EIRIQCKID |
| 14380 | EIRPWDCKW |
| 14381 | EIRTIDWQW |
| 14382 | EISDIHDLR |
| 14383 | EISEYCQPS |
| 14384 | EISPYNDVE |
| 14385 | EISQIPPGW |
| 14386 | EITCLCVHS |
| 14387 | EITDFQYEN |
| 14388 | EITDKDVCT |
| 14389 | EITEFWGNY |
| 14390 | EITEITMSK |
| 14391 | EITPKDSHT |
| 14392 | EIVDFDNYF |
| 14393 | EIVEQCGNR |
| 14394 | EIVHQDDVN |
| 14395 | EIYEFQMWD |
| 14396 | EIYEFRASN |
| 14397 | EKQMDGINH |
| 14398 | EKYFKMFDN |
| 14399 | ELRCTKLQN |
| 14400 | ELRIQCKID |
| 14401 | ELSDSMDSN |
| 14402 | EMHECPMAW |
| 14403 | EMKCIDVED |
| 14404 | EMRTYNKDY |
| 14405 | ENYPWEQLE |
| 14406 | EPRCENYME |
| 14407 | EQCRGEPDE |
| 14408 | EQKDNMMMN |
| 14409 | ERRCAWFNA |
| 14410 | ESDEREEAD |
| 14411 | ESDPINEAN |
| 14412 | ESGCKMWPT |
| 14413 | ESHEIRDHN |
| 14414 | ESQEAEWGW |
| 14415 | ESRCGDKGC |
| 14416 | ESRCVDNTV |
| 14417 | ESTDFDELN |
| 14418 | ESTELRTGN |
| 14419 | ESTNIPMAF |
| 14420 | ESVPKHEYM |
| 14421 | ETAGKSIHH |
| 14422 | ETEDYRTSN |
| 14423 | ETEEIQKGH |
| 14424 | ETHEFRASN |
| 14425 | ETMDKLWAN |
| 14426 | ETMNQSWGL |
| 14427 | ETNEEEDGN |
| 14428 | ETQMFEIGE |
| 14429 | EVACFDDKN |
| 14430 | EVACLGVIN |
| 14431 | EVAETDNFF |
| 14432 | EVAHENFRM |
| 14433 | EVAHLDATK |
| 14434 | EVAPECKFE |
| 14435 | EVCDLIHDN |
| 14436 | EVCEEEFDP |
| 14437 | EVDDKYHED |
| 14438 | EVDEGHNNE |
| 14439 | EVDGRNRGQ |
| 14440 | EVDPLEIAL |
| 14441 | EVECRHETC |
| 14442 | EVEELWNPF |
| 14443 | EVEEMNWGK |
| 14444 | EVEEQIELI |
| 14445 | EVESLPCSL |
| 14446 | EVEWSCRFE |
| 14447 | EVGGMGEVK |
| 14448 | EVGIQCKID |
| 14449 | EVGIQCKVD |
| 14450 | EVGPLNEWG |
| 14451 | EVGTHDCLD |
| 14452 | EVHDFHYIP |
| 14453 | EVHDKNTQF |
| 14454 | EVHEARPYF |
| 14455 | EVHEFRASN |
| 14456 | EVHELENIN |
| 14457 | EVHMIPEMQ |
| 14458 | EVHTEDYGK |
| 14459 | EVHVSEIMK |
| 14460 | EVIEWCNYE |
| 14461 | EVIMQCMMK |
| 14462 | EVIPTCHFG |
| 14463 | EVKPKDEAH |
| 14464 | EVKQWNEVM |
| 14465 | EVLIQCKID |
| 14466 | EVMHWNIEW |
| 14467 | EVMMFSCYE |
| 14468 | EVMSLESFE |
| 14469 | EVMYRCLMP |
| 14470 | EVPEIEHDK |
| 14471 | EVPIQCKID |
| 14472 | EVPTRDCCD |
| 14473 | EVQCDQIHP |
| 14474 | EVQDDSKAN |
| 14475 | EVQDDYESN |
| 14476 | EVQDLDMMD |
| 14477 | EVQDTDCSP |
| 14478 | EVQEDCMFE |
| 14479 | EVQIQCKID |
| 14480 | EVRCFHELE |
| 14481 | EVREKEPND |
| 14482 | EVRESDFRE |
| 14483 | EVRFEMRYE |
| 14484 | EVRIECKID |
| 14485 | EVRIHCKLD |
| 14486 | EVRIPCKID |
| 14487 | EVRIQCEID |
| 14488 | EVRIQCKFD |
| 14489 | EVRIQCKFG |
| 14490 | EVRIQCKIN |
| 14491 | EVRIQCKIV |
| 14492 | EVRIQCKIY |
| 14493 | EVRIQCKLD |
| 14494 | EVRIQCKLV |
| 14495 | EVRIQCKMD |
| 14496 | EVRIQCQID |
| 14497 | EVRIQCTID |
| 14498 | EVRIQFKID |
| 14499 | EVRIQWKID |
| 14500 | EVRIRCKID |
| 14501 | EVRIRCKMD |
| 14502 | EVRIYCKID |
| 14503 | EVRMQCKID |
| 14504 | EVRNKCKID |
| 14505 | EVRNQCKID |
| 14506 | EVRSECKID |
| 14507 | EVRSKCKID |
| 14508 | EVRSQCKID |
| 14509 | EVRTKCKID |
| 14510 | EVRTKNEAW |
| 14511 | EVRTQCKID |
| 14512 | EVRTQIIMD |
| 14513 | EVRVQCKID |
| 14514 | EVRYNPFHW |
| 14515 | EVSELDGAW |
| 14516 | EVSEVKENG |
| 14517 | EVSGTDELI |
| 14518 | EVSLWEIGC |
| 14519 | EVTDPNMLD |
| 14520 | EVTDYSGMF |
| 14521 | EVTEGCPMG |
| 14522 | EVTPMQDID |
| 14523 | EVTQIWLFP |
| 14524 | EVVDCNWLM |
| 14525 | EVVEFHWQQ |
| 14526 | EVVVRNMPT |
| 14527 | EVWIQCKID |
| 14528 | EVYDHDTYI |
| 14529 | EVYHICGAN |
| 14530 | EWIWCYDRG |
| 14531 | EWTMFIEAH |
| 14532 | FCDCGHWPN |
| 14533 | FCECGHWPN |
| 14534 | FDCHTFGYR |
| 14535 | FDECFPEYW |
| 14536 | FDKMFREIL |
| 14537 | FDRCRNANW |
| 14538 | FDRPGRNIL |
| 14539 | FEDCGWDKT |
| 14540 | FEIGGKTRT |
| 14541 | FENCRDAGR |
| 14542 | FERCEGDNH |
| 14543 | FHCICAMGH |
| 14544 | FHSLCAMGH |
| 14545 | FKFEPWEYW |
| 14546 | FKHSLRQWT |
| 14547 | FKHSQREWT |
| 14548 | FKHSRREWT |
| 14549 | FKKEDAGQF |
| 14550 | FKQDDRSEV |
| 14551 | FKVLPWWYH |
| 14552 | FLRCKKWHI |
| 14553 | FMRPCDMGM |
| 14554 | FNRCGRPKN |
| 14555 | FNVDCGTWF |
| 14556 | FPAFRGGSF |
| 14557 | FPIDFVPFR |
| 14558 | FPKASECNW |
| 14559 | FPKECECNW |
| 14560 | FPKGSECTW |
| 14561 | FPKHDWPTS |
| 14562 | FPPFCWSGM |
| 14563 | FPPWPLDLV |
| 14564 | FPRFGDDRM |
| 14565 | FPRGSECNW |
| 14566 | FPSCHOCNI |
| 14567 | FQPWPKYWC |
| 14568 | FQWRSEMKN |
| 14569 | FRHSLREWT |
| 14570 | FRMENRNAN |
| 14571 | FRQSPWPVV |
| 14572 | FRVTPKPEA |
| 14573 | FSDPKNMSI |
| 14574 | FWECCTDSG |
| 14575 | FWGCNWDPK |
| 14576 | FWQCPWQED |
| 14577 | GGDWDFGEW |
| 14578 | GKSADLLKQ |
| 14579 | GPDCECQRG |
| 14580 | GQPTPWLDH |
| 14581 | GRDCKYINW |
| 14582 | GRGCQIEKY |
| 14583 | GRPCCGYNY |
| 14584 | GRPCEPILQ |
| 14585 | GRYCECVEH |
| 14586 | GSDEFMAYG |
| 14587 | GTMNLSWGL |
| 14588 | GVCDDHKPN |
| 14589 | GWECCTDSG |
| 14590 | HARCVDNTV |
| 14591 | HCECGHWPN |
| 14592 | HDCKLKPIM |
| 14593 | HDRCFASYC |
| 14594 | HDRCFDSTA |
| 14595 | HGVGRSGDN |
| 14596 | HHFHVSLDI |
| 14597 | HHFQVNLDI |
| 14598 | HHFYVNLDI |
| 14599 | HHYHVNLDI |
| 14600 | HKGMEKNPV |
| 14601 | HKYIDWIDG |
| 14602 | HPFHVNLDI |
| 14603 | HPKCSEQYR |
| 14604 | HPRCATDHA |
| 14605 | HQRCPDRAD |
| 14606 | HRQCNLLDI |
| 14607 | HRSCDRMIM |
| 14608 | HSDPKNMSI |
| 14609 | HTPDRTEFE |
| 14610 | HTRCVDHDT |
| 14611 | HVDDLNDWA |
| 14612 | HVKCLDYQI |
| 14613 | IDICGIEHR |
| 14614 | IDRMGMILN |
| 14615 | IDYISFVDR |
| 14616 | IGIGRSGDN |
| 14617 | IIAQRDQLN |
| 14618 | IIGDFSQLG |
| 14619 | IIKEMDIHW |
| 14620 | IIPDREDIS |
| 14621 | IITQRDQVN |
| 14622 | IMDCDTNKY |
| 14623 | INDISFVDR |
| 14624 | INFISFVDR |
| 14625 | INYISFDDR |
| 14626 | INYISFIDR |
| 14627 | INYISFVAR |
| 14628 | INYISFVDP |
| 14629 | INYISFVDS |
| 14630 | INYISFVNR |
| 14631 | INYSSFVDR |
| 14632 | ISYISFVDR |
| 14633 | ITYISFVDR |
| 14634 | IVDQWNMGI |
| 14635 | IWDCMVEKF |
| 14636 | KDDCGTQER |
| 14637 | KDDFDPDQG |
| 14638 | KDEIEDFKS |
| 14639 | KDKFEWLTH |
| 14640 | KDWRGWEER |
| 14641 | KDWWEDDKQ |
| 14642 | KDWYAWEEW |
| 14643 | KEGCETDKS |
| 14644 | KEMCQWDAQ |
| 14645 | KRDCPWHNL |
| 14646 | KSRCADNTV |
| 14647 | KVIEFDMDI |
| 14648 | LAECMEEMC |
| 14649 | LCDYSWWRY |
| 14650 | LCPCRLWDW |
| 14651 | LCTDKMDGD |
| 14652 | LDECCYDTR |
| 14653 | LDEYARPQQ |
| 14654 | LDRCLVIVQ |
| 14655 | LDWAEHFNC |
| 14656 | LEFGGKTRT |
| 14657 | LEIGAKTRT |
| 14658 | LEIGGKTKT |
| 14659 | LERCLGLNQ |
| 14660 | LESAGKTRT |
| 14661 | LHECRKEIH |
| 14662 | LKDCHGDGR |
| 14663 | LKGCPCDTM |
| 14664 | LNCQCWPER |
| 14665 | LNGCDSRMR |
| 14666 | LTDGWDPER |
| 14667 | LWQECDEAW |
| 14668 | MDKEIETWP |
| 14669 | MDPQDEMKF |
| 14670 | MDRCPPERT |
| 14671 | MDSCYDRVR |
| 14672 | MDWFKGERS |
| 14673 | MEGSCKQER |
| 14674 | MEILGTMKQ |
| 14675 | MEQQPGPKR |
| 14676 | MGDCQTLSY |
| 14677 | MHPRPRPEV |
| 14678 | MIDFREDVQ |
| 14679 | MIEPYEMHY |
| 14680 | MINDMKMFF |
| 14681 | MKWINIIDW |
| 14682 | MMFENHORN |
| 14683 | MNRQRDHIR |
| 14684 | MPKFEDVDH |
| 14685 | MQIECTWSH |
| 14686 | MQLDCTWHN |
| 14687 | MRGCMDKTR |
| 14688 | MSDEWMCNN |
| 14689 | MTRCVDADY |
| 14690 | MVEEQEEND |
| 14691 | MVEICCSNK |
| 14692 | MVMIWDHDQ |
| 14693 | MVNPIDWQC |
| 14694 | MVRCVEGDD |
| 14695 | MYECGNIGE |
| 14696 | NCECGHWPN |
| 14697 | NERWDHDQL |
| 14698 | NESIDDIEQ |
| 14699 | NHWNQELDF |
| 14700 | NIALRDSMN |
| 14701 | NIDAWDNHN |
| 14702 | NIVDDQYGN |
| 14703 | NKPFDAFKW |
| 14704 | NKWHPMIVI |
| 14705 | NLDCRPVNG |
| 14706 | NMFPLKPQN |
| 14707 | NMFQLKPQH |
| 14708 | NMFQLKPQT |
| 14709 | NMIQLKPQN |
| 14710 | NNMLIRPNF |
| 14711 | NSDPKNMPI |
| 14712 | NSDPKNMSI |
| 14713 | NVNEWEECE |
| 14714 | NVREYDRYE |
| 14715 | NVVEQHEYN |
| 14716 | NWEFNARND |
| 14717 | PAEGGNVSD |
| 14718 | PAFCQRPEQ |
| 14719 | PCAMHNWGF |
| 14720 | PCDMHNWGC |
| 14721 | PCGMHNWGC |
| 14722 | PCGPIEYDE |
| 14723 | PCPCDHGGK |
| 14724 | PCSQPIHEP |
| 14725 | PCTMPYVEN |
| 14726 | PCTMTYVED |
| 14727 | PDCLRPVMK |
| 14728 | PDHCDPMPE |
| 14729 | PDHCVKNER |
| 14730 | PDICVWHDN |
| 14731 | PDIDMDMDY |
| 14732 | PDLRPRDAC |
| 14733 | PDRFPQIKS |
| 14734 | PDTEKEMRF |
| 14735 | PDTYVRERY |
| 14736 | PEAGDQFKT |
| 14737 | PEEMKYPLK |
| 14738 | PEVFTDIRP |
| 14739 | PEWGTSCNW |
| 14740 | PFACDGYEM |
| 14741 | PGGCRGIRY |
| 14742 | PGICGEPGK |
| 14743 | PGICVWHDN |
| 14744 | PGKCANERM |
| 14745 | PGPCVMEIW |
| 14746 | PHFHVNLDI |
| 14747 | PHPCMFPTM |
| 14748 | PICQGMMEN |
| 14749 | PIICVWHDN |
| 14750 | PKGSNMFDW |
| 14751 | PKKVFCPEE |
| 14752 | PKQCKVHEQ |
| 14753 | PKSCKFIDT |
| 14754 | PKYFECIYY |
| 14755 | PMDCGKHKK |
| 14756 | PMFHPRQYP |
| 14757 | PMMISYIQR |
| 14758 | PMRCGDDAK |
| 14759 | PNCKQWVDN |
| 14760 | PNDLKFETF |
| 14761 | PNICPLDEQ |
| 14762 | PNKLSGGEE |
| 14763 | PNRCHDDRG |
| 14764 | PPVRPWEEM |
| 14765 | PQFFSWSEY |
| 14766 | PQHAKCGEF |
| 14767 | PQRVLPMEY |
| 14768 | PREFEWYPH |
| 14769 | PRGCDCLOS |
| 14770 | PRRCDNWKT |
| 14771 | PSGPIQYDE |
| 14772 | PSKCEWTDN |
| 14773 | PSRCFDNTV |
| 14774 | PSRCVDNTV |
| 14775 | PTMNLSWGL |
| 14776 | PTNEEEDGN |
| 14777 | PVACVWHDN |
| 14778 | PVFCVWHDN |
| 14779 | PVGFDPPPF |
| 14780 | PVHDFDAAY |
| 14781 | PVHSHNIDN |
| 14782 | PVICDWHDN |
| 14783 | PVICGWHDN |
| 14784 | PVICGWHEN |
| 14785 | PVICLWHDN |
| 14786 | PVICVWHAN |
| 14787 | PVICVWHDH |
| 14788 | PVICVWHDS |
| 14789 | PVICVWHDT |
| 14790 | PVICVWHGN |
| 14791 | PVICVWHHN |
| 14792 | PVICVWHNN |
| 14793 | PVICVWPDN |
| 14794 | PVICVWYDN |
| 14795 | PVIFVWHDN |
| 14796 | PVIYIWHDN |
| 14797 | PVLCVWHDK |
| 14798 | PVLCVWHDN |
| 14799 | PVMCVWHDN |
| 14800 | PVNCVWHDN |
| 14801 | PVQCNWERE |
| 14802 | PVRCTKPRD |
| 14803 | PVSCVWHDN |
| 14804 | PVTCVWHDN |
| 14805 | PVVCVWHDN |
| 14806 | PWLDDSEME |
| 14807 | PWLWHKSAC |
| 14808 | PWQCKGPIN |
| 14809 | PWQFCYPWT |
| 14810 | PWRVQKHGV |
| 14811 | PWRWHKSAW |
| 14812 | PWRWHKSDC |
| 14813 | PWRWHKSPC |
| 14814 | PWRWHKSSC |
| 14815 | PWRWHKSTC |
| 14816 | PWRWHKSVC |
| 14817 | PWRWHRSAC |
| 14818 | PWRWRKSAC |
| 14819 | PWRWYKSAC |
| 14820 | PWSCQPEQA |
| 14821 | PWTYDKEFY |
| 14822 | PWVFTYPSN |
| 14823 | PYGPFEYDE |
| 14824 | PYGWRNIDQ |
| 14825 | PYIMEFHAF |
| 14826 | PYIMEFHPC |
| 14827 | PYIMEFHPV |
| 14828 | PYIMEFHTC |
| 14829 | PYSMEFHPF |
| 14830 | QANEEEDGN |
| 14831 | QCDCSWWRY |
| 14832 | QCDYSWWRD |
| 14833 | QCDYSWWRH |
| 14834 | QCDYSWWRN |
| 14835 | QDGTGEPKG |
| 14836 | QFRCVDNKV |
| 14837 | QIEFIWDAH |
| 14838 | QIEMTEISQ |
| 14839 | QISEGEVLN |
| 14840 | QKNEEEDGN |
| 14841 | QKPCKVEQV |
| 14842 | QKRCCWHRC |
| 14843 | QLDSWVEKR |
| 14844 | QMEEIQIGH |
| 14845 | QNLEIIGAG |
| 14846 | QPICRRDKR |
| 14847 | QPMEWKMVR |
| 14848 | QPRCLDNTV |
| 14849 | QPRCYRNIS |
| 14850 | QSDEYNIAF |
| 14851 | QSRCFDNTV |
| 14852 | QSRCLDNTL |
| 14853 | QSRCVDITV |
| 14854 | QSRCVDNPG |
| 14855 | QSRCVDNRV |
| 14856 | QSRCVDNSV |
| 14857 | QSRCVDTTV |
| 14858 | QTHEEDDGN |
| 14859 | QTHEEEDGN |
| 14860 | QTNDKEDGN |
| 14861 | QTNEEEAGN |
| 14862 | QTNEEEDAN |
| 14863 | QTNEEEDGH |
| 14864 | QTNEEEDGT |
| 14865 | QTNEEEDSN |
| 14866 | QTNEEEDVN |
| 14867 | QTNEGEDGN |
| 14868 | QTNEQEDGN |
| 14869 | QTSVEEDGN |
| 14870 | QTTEEEDGN |
| 14871 | QTYEEEDGN |
| 14872 | QVGDDRSFA |
| 14873 | QVRIQCKID |
| 14874 | QWDDDQWST |
| 14875 | QWPDDQWST |
| 14876 | QYRCSDSAF |
| 14877 | RDCCDAWSR |
| 14878 | RDECRMESG |
| 14879 | RDKCRMESG |
| 14880 | RDPCRMESG |
| 14881 | RDPLNDSRY |
| 14882 | RDQCRMEAG |
| 14883 | RDQCRMECG |
| 14884 | RDQCRMEDG |
| 14885 | RDQCRMEFG |
| 14886 | RDQCRMEHG |
| 14887 | RDQCRMESA |
| 14888 | RDQCRMESD |
| 14889 | RDQCRMESR |
| 14890 | RDQCRMESS |
| 14891 | RDQCRMESV |
| 14892 | RDQCRMETC |
| 14893 | RDQCRMEYG |
| 14894 | RDQCRMKSG |
| 14895 | RDQCRVESG |
| 14896 | RDSHDEFEW |
| 14897 | REDCWPPEN |
| 14898 | REHIGFMKF |
| 14899 | REIGGKTRT |
| 14900 | REKCHQHQC |
| 14901 | RSRCVDNTV |
| 14902 | RWGCDLPQI |
| 14903 | RYQCRMESG |
| 14904 | SADIFAIND |
| 14905 | SCECGHWPI |
| 14906 | SCECGHWPN |
| 14907 | SDPGEILKR |
| 14908 | SGDGDFGEW |
| 14909 | SGDLDFGEW |
| 14910 | SGDWAFGEW |
| 14911 | SGDWDFGKW |
| 14912 | SGDWNFGEW |
| 14913 | SGRCPFSSK |
| 14914 | SIEECCEEM |
| 14915 | SMFQLKPQN |
| 14916 | SNDACIQSF |
| 14917 | SNECWNEMA |
| 14918 | SNGDCIQAF |
| 14919 | SNRCHDDRG |
| 14920 | SNVDCIQSF |
| 14921 | SPERMDYDG |
| 14922 | SPKGSECNW |
| 14923 | SQQWCEQPD |
| 14924 | SRPCCKDIR |
| 14925 | SSDPKNMSI |
| 14926 | STMDLSWGL |
| 14927 | STMNLSWGF |
| 14928 | SVDYEDEVW |
| 14929 | SVEPKSMTG |
| 14930 | SVETLDQSL |
| 14931 | SVICVWHDN |
| 14932 | SVLRRDEVN |
| 14933 | SVMDIDCHQ |
| 14934 | SWLDNILPK |
| 14935 | SYFWCFMDC |
| 14936 | TADIFAIND |
| 14937 | TAEIFAVND |
| 14938 | TDKYCDQKR |
| 14939 | TFSEIRDQC |
| 14940 | THPWEMTKE |
| 14941 | THRCHDDRG |
| 14942 | TIDSWFQER |
| 14943 | TIGEINDAN |
| 14944 | TKFHDGDWC |
| 14945 | TKMLDRDAE |
| 14946 | TNRCHDDRC |
| 14947 | TNRCHDDRD |
| 14948 | TNRCHDDRV |
| 14949 | TNRCHDHRG |
| 14950 | TNRCHDYRG |
| 14951 | TPPWESIRK |
| 14952 | TQPFSKDED |
| 14953 | TREHQIISP |
| 14954 | TRPCGTLSH |
| 14955 | TTHEGHEDN |
| 14956 | TVICVWHDN |
| 14957 | TVPCMWQTY |
| 14958 | TVWEQDLLE |
| 14959 | TWAPWCCDC |
| 14960 | TWAPWCCYY |
| 14961 | TWGPWCCYF |
| 14962 | TWPPWCCYF |
| 14963 | TWRCGDIGH |
| 14964 | TYRCHDDRG |
| 14965 | VADCKQPRF |
| 14966 | VDCSKILRR |
| 14967 | VDDRPKPEH |
| 14968 | VDRLFRMEF |
| 14969 | VDRQVRMEY |
| 14970 | VDSHKIIRR |
| 14971 | VEIRGKTRT |
| 14972 | VHEGKTFDI |
| 14973 | VHODKIIGE |
| 14974 | VICEFEEGN |
| 14975 | VITEDDQAE |
| 14976 | VNEWEADAE |
| 14977 | VQLCDLLRR |
| 14978 | VRYSDMFWW |
| 14979 | WAEHDFPKG |
| 14980 | WCDDDMRRQ |
| 14981 | WCTEEWEEK |
| 14982 | WDCSMTKKH |
| 14983 | WDFSGRIDN |
| 14984 | WDHREERTH |
| 14985 | WDICSGLRQ |
| 14986 | WDKCIPMHW |
| 14987 | WDLVDPPKY |
| 14988 | WDMNGKDGS |
| 14989 | WDPRRPVKK |
| 14990 | WDRCLELNK |
| 14991 | WDSLEWDRA |
| 14992 | WDYSMPKKH |
| 14993 | WDYSMSKKH |
| 14994 | WDYSMTKKN |
| 14995 | WDYSMTKKP |
| 14996 | WEFTMAPEV |
| 14997 | WEKCGWPPI |
| 14998 | WELCMELNQ |
| 14999 | WEPCLELNQ |
| 15000 | WERCLELDQ |
| 15001 | WERCLELHQ |
| 15002 | WERCLELNL |
| 15003 | WERCLELNP |
| 15004 | WERCLEQNH |
| 15005 | WERCLEVNQ |
| 15006 | WERCLGLNQ |
| 15007 | WERCLGLTQ |
| 15008 | WERCLQLNQ |
| 15009 | WERCLVLNQ |
| 15010 | WERCMELNQ |
| 15011 | WERCQELNH |
| 15012 | WGAFRGVCE |
| 15013 | WHGFHKIMF |
| 15014 | WIAPRHMGL |
| 15015 | WKAFRGGIG |
| 15016 | WKDCKQMKM |
| 15017 | WKFGDKLIM |
| 15018 | WKGAPALER |
| 15019 | WKHCEREIN |
| 15020 | WKIDPCVLQ |
| 15021 | WKLGDGIQD |
| 15022 | WKRPPGGEH |
| 15023 | WKSHEWDPH |
| 15024 | WLECNPPEQ |
| 15025 | WLHPYSYCA |
| 15026 | WNLQDKPGE |
| 15027 | WNPARKDDI |
| 15028 | WNSCPWQLP |
| 15029 | WPCHDAPIW |
| 15030 | WPIGEKMVF |
| 15031 | WPKERVPKG |
| 15032 | WPNDDMTRM |
| 15033 | WQFTGKPTL |
| 15034 | WQHIMKPQI |
| 15035 | WQMLCRPPV |
| 15036 | WQRCLELNQ |
| 15037 | WRQFPCAEQ |
| 15038 | WRSCPKGLV |
| 15039 | WSRCVDNTV |
| 15040 | WTIEPEWYF |
| 15041 | WTKDDEQLE |
| 15042 | WVHPFSYCA |
| 15043 | WVHPYTYCA |
| 15044 | WVHQYSYCA |
| 15045 | WVHTYSYCA |
| 15046 | WVLDISHCG |
| 15047 | WVRCLELKQ |
| 15048 | WVYPYSYCA |
| 15049 | WWRVPCESH |
| 15050 | WYDWIHERT |
| 15051 | WYICQKDSM |
| 15052 | WYKCQDVDR |
| 15053 | YADPKNMSI |
| 15054 | YCECEHWPN |
| 15055 | YCECGHCPI |
| 15056 | YCECGHSPN |
| 15057 | YCECGHWPD |
| 15058 | YCECGHWPH |
| 15059 | YCECGHWPI |
| 15060 | YCECGHWPY |
| 15061 | YCECGHWQN |
| 15062 | YCECGHWSN |
| 15063 | YCECGHWTS |
| 15064 | YCECGNWPN |
| 15065 | YCECGRWPI |
| 15066 | YCECGRWPN |
| 15067 | YCECGYWPN |
| 15068 | YCEGGHWPN |
| 15069 | YCGCGIHQQ |
| 15070 | YDESVRISR |
| 15071 | YEEFPEIIM |
| 15072 | YEETRVEAR |
| 15073 | YELYQDIDN |
| 15074 | YERCHHQGD |
| 15075 | YGPCCHFQF |
| 15076 | YHKFGDMDI |
| 15077 | YHRCDEFFL |
| 15078 | YIRPEQPVC |
| 15079 | YKDRDICRL |
| 15080 | YKEHEAVQF |
| 15081 | YKFYPGLPT |
| 15082 | YKGMEKNAG |
| 15083 | YKGMEKNRV |
| 15084 | YKGMEKTPV |
| 15085 | YKMCDHDDC |
| 15086 | YKPCMTIDV |
| 15087 | YKQHEGEQF |
| 15088 | YPHCRLLGP |
| 15089 | YPHMDCSEE |
| 15090 | YRDCEQMEV |
| 15091 | YRDCRQFNR |
| 15092 | YRVLPRIKD |
| 15093 | YSDAKNMSI |
| 15094 | YSDGKNMSI |
| 15095 | YSDHQNMSI |
| 15096 | YSDPKDMSI |
| 15097 | YSDPKNISM |
| 15098 | YSDPKNMFI |
| 15099 | YSDPKNMPI |
| 15100 | YSDPKNMSV |
| 15101 | YSDPKNMTL |
| 15102 | YSDPKNMTT |
| 15103 | YSDPKNMYI |
| 15104 | YSDPKTMSI |
| 15105 | YSDTKKMSI |
| 15106 | YSGPKNMSI |
| 15107 | YSHPKHMSI |
| 15108 | YSYPKNMTI |
| 15109 | YVDPYEMYG |
| 15110 | YVFGQKDEM |
| 15111 | YVIEQKDEM |
| 15112 | YVIGQKDEV |
| 15113 | YVLDSWRTS |
| 15114 | YWECGHWPN |
| 15115 | YWGCPDQVR |
| 15116 | YYNHKIFEP |
| 15117 | YYNPKIVEP |

### Example 35

AAV5 Variants with Lymph Node Tissue Tropism as Identified by Machine Learning

This example describes preferred properties of engineered AAV5 variants that display lymph node tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display lymph node tissue tropism.

Favored biophysical properties and favored amino acid residues at each position in the 581 to 589 region of an AAV5 VP capsid polypeptide, which are associated with a higher probability of lymph node tissue tropism are described below. Any of the below machine learning-derived positional amino acid preferences described in TABLE 54 can be present alone or in combination with each other or in combination with any of the amino acid preferences observed and described in EXAMPLE 16, TABLE 31. Listed below in TABLE 54 are a summary of positional features shared between the top important features for lymph node tropism extracted from the ML models.

TABLE 54
Machine Learning-Derived Lymph Node Tissue Tropism Rules

High average flexibility at Xaa1
Xaa1 is selected from D, E, P, G, Q, S, or R
High hbond donors at Xaa1
Xaa1 is selected from R
High mol mass at Xaa1
Xaa1 is selected from Y, W, R, or F
Low solubility at Xaa2
Xaa2 is selected from N or E
Low average flexibility at Xaa3
Xaa3 is selected from W, M, or F
Low mutability at Xaa3
Xaa3 is selected from R, H, K, P, Y, F, L, or C
Low mutability at Xaa4
Xaa4 is selected from C
High mutability at Xaa5
Xaa5 is selected from N
Medium mol mass at Xaa5
Xaa5 is selected from D, I, L, or N
High mol mass at Xaa6
Xaa6 is selected from Y, W, R, or F
High average flexibility at Xaa6
Xaa6 is selected from G or R
High average flexibility at Xaa7
Xaa7 is selected from D, E, K, P, I, N, Q, or S
Low solubility at Xaa7
Xaa7 is selected from N or E
Low solubility at Xaa8
Xaa8 is selected from N, E, or D
Medium mutability at Xaa8
Xaa8 is selected from R or H
Low mutability at Xaa9
Xaa9 is selected from P or K
High average flexibility at Xaa9
Xaa9 is selected from D, E, P, or S
High solubility at Xaa9
Xaa9 is selected from M or V

TABLE 55 below provides sequences of the 581 to 589 region for variant AAV VP1 capsid polypeptides that exhibited lymph node tissue tropism and comport to one or more of the rules provided in TABLE 54. The present disclosure, thus, provides for rAAVs composed of engineered AAV5 VP1 capsid polypeptides having a SEQ ID NO: 2, wherein the Xaa1 to Xaa9 region is any one of SEQ ID NO: 20118-SEQ ID NO: 21117, as disclosed in TABLE 55. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 55
Sequences of the 581 to 589 Region in AAV5
VP1 Capsid Polypeptide that Drive Lymph
Node Tissue Tropism

| SEQ ID | 581-589 |
|---|---|
| NO | Sequence |
| 20118 | ACKLDHMPP |
| 20119 | ADCCSCKAN |
| 20120 | ADDCTHKNH |
| 20121 | ADSCTHKNH |
| 20122 | ADYCAHKNH |
| 20123 | ADYCPHKNH |
| 20124 | ADYCTHKTH |
| 20125 | ADYCTHRNH |
| 20126 | ADYGTHKNH |
| 20127 | ADYWTHKNH |
| 20128 | AECCSIFSS |
| 20129 | AERLCIEWD |
| 20130 | AESKCDIGT |
| 20131 | AESRCDVGT |
| 20132 | AEWSWAEHS |
| 20133 | AFWGTDVWG |
| 20134 | AFWSPDVWG |
| 20135 | AGFRIDFRC |
| 20136 | AGGWLFFWH |
| 20137 | AGNNEDTQL |
| 20138 | AGQWGSLNP |
| 20139 | AGSNEDKQL |
| 20140 | AGTDQTMHM |
| 20141 | AHRWSWSES |
| 20142 | AISQDMFMQ |
| 20143 | AISQDMLMQ |
| 20144 | AMLCNRVCE |
| 20145 | ANCCSCKAD |
| 20146 | ANCCSCKDN |
| 20147 | ANCCSCKPN |
| 20148 | ANCCSCKTN |
| 20149 | ANCCSCKTS |
| 20150 | ANCCSGKAN |
| 20151 | ANCYSCKAN |
| 20152 | ASASHDFMG |
| 20153 | ASCEDEGIL |
| 20154 | ASGSNDFMG |
| 20155 | ATDRVVVNF |
| 20156 | ATSRCDVQT |
| 20157 | AVPWEWQEG |
| 20158 | CCAALIQSG |
| 20159 | CCDALIQSS |
| 20160 | CCDDLIQSG |
| 20161 | CCDPLIQSG |
| 20162 | CDMWWYQDP |
| 20163 | CEKYNLPKI |
| 20164 | CKDRPIIDC |
| 20165 | CLREAVVGI |
| 20166 | CVLNMEYCL |
| 20167 | CVQRLLFKC |
| 20168 | CVVNMEYYL |
| 20169 | DAAIKMDES |
| 20170 | DCIFFHCPK |
| 20171 | DDFGPNHRH |
| 20172 | DDHQPMQSR |
| 20173 | DDRGFTRSM |
| 20174 | DDWSWAEHS |
| 20175 | DECIMQRSK |
| 20176 | DEFDPNHRH |
| 20177 | DEFGPNHGH |
| 20178 | DEFGPNHIH |
| 20179 | DEFGPNHKH |
| 20180 | DEFGPNHRR |
| 20181 | DEFGPNHRY |
| 20182 | DEFGPNYRH |
| 20183 | DEFGPSHRH |
| 20184 | DEFGTNHRH |
| 20185 | DEIGPNHRH |
| 20186 | DEMGYDQLD |
| 20187 | DERCYDQLD |
| 20188 | DEWAWAEHS |
| 20189 | DEWPWAEHS |
| 20190 | DEWSGAEHS |
| 20191 | DEWSLAEHS |
| 20192 | DEWSWAAHS |
| 20193 | DEWSWAEHA |
| 20194 | DEWSWAERS |
| 20195 | DEWSWAGHS |
| 20196 | DEWSWAQHS |
| 20197 | DEWSWSEHS |
| 20198 | DEWSWTEHS |
| 20199 | DEWSWVEHS |
| 20200 | DEWTWAEHS |
| 20201 | DEWWSGHAH |
| 20202 | DEWWSGHAP |
| 20203 | DEYGPNHRH |
| 20204 | DFHSRTNIN |
| 20205 | DFLDKSTFF |
| 20206 | DFLHGTSIM |
| 20207 | DFLHVTSTM |
| 20208 | DFLPVTSIM |
| 20209 | DFLYVTSIM |
| 20210 | DGEWGSLNP |
| 20211 | DGFRIEFRC |
| 20212 | DGLKNDDRK |
| 20213 | DGNDQTMHM |
| 20214 | DGPDQTMHM |
| 20215 | DGQWGSLNL |
| 20216 | DGQWGSLNQ |
| 20217 | DGQWGSLNS |
| 20218 | DGTDPTMHM |
| 20219 | DGTDQTMHV |
| 20220 | DGTGQTMHM |
| 20221 | DGTNQTMHM |
| 20222 | DHQGPKLSK |
| 20223 | DMYMQQIWR |
| 20224 | DNNSPQEAS |
| 20225 | DNRCYKYWN |
| 20226 | DPICKVEDW |
| 20227 | DPIWLIKEC |
| 20228 | DPIWRVEDW |
| 20229 | DPIWTVEDW |
| 20230 | DPKWANCDC |
| 20231 | DPMWEREMF |
| 20232 | DPSWKVEDW |
| 20233 | DPVWKVEDW |
| 20234 | DQDAGGYDS |
| 20235 | DQWSWAEHS |
| 20236 | DRQWGSLNP |
| 20237 | DSKNQNCFK |
| 20238 | DSLHVTSIM |
| 20239 | DSNYQGTKH |
| 20240 | DSPNQNCFK |
| 20241 | DSQNENCFK |
| 20242 | DSQNQKCFK |
| 20243 | DSQNQNRFK |
| 20244 | DSRNQNCFK |
| 20245 | DTFGLRFDD |
| 20246 | DTFGLRIDD |
| 20247 | DTFGQRVDD |
| 20248 | DTFWLRVDD |
| 20249 | DTQNQNCFK |
| 20250 | DTRCDKYWN |
| 20251 | DTRCSKYWN |
| 20252 | DTRCYKYWT |
| 20253 | DVQWGSLNP |
| 20254 | DWTDQTMHM |
| 20255 | EAHTNSIGK |
| 20256 | EANWVYIEA |
| 20257 | EARTNSICK |
| 20258 | ECEQDDTDI |
| 20259 | ECGLWNFSG |
| 20260 | ECKITESLT |
| 20261 | EDHLHGPGD |
| 20262 | EDHWLGPGD |
| 20263 | EDHWRGPGD |
| 20264 | EDRWHGPGD |
| 20265 | EDYWHGPGD |
| 20266 | EEAHSVKFV |
| 20267 | EEANSVKFA |
| 20268 | EEARCVNPC |
| 20269 | EEAYSVKFA |
| 20270 | EEDHSVKFA |
| 20271 | EEFARIPGN |
| 20272 | EEFDRIPGH |
| 20273 | EEFSRIPGH |
| 20274 | EEPETYTQM |
| 20275 | EEPRCGNPC |
| 20276 | EEPRCVDPC |
| 20277 | EEPRCVNPG |
| 20278 | EEPRCVNPW |
| 20279 | EEPRCVNPY |
| 20280 | EEPRCVNTC |
| 20281 | EEPRCVSPC |
| 20282 | EEPRCVTPC |
| 20283 | EFCENLTAI |
| 20284 | EFCENMTSI |
| 20285 | EFFCEADKA |
| 20286 | EFFCPADKA |
| 20287 | EFFCQADKT |
| 20288 | EFFCQADKV |
| 20289 | EFFCQANKA |
| 20290 | EFFCQVDKA |
| 20291 | EFFFQADKA |
| 20292 | EFQQDDTDI |
| 20293 | EGIKFQTEL |
| 20294 | EGKYMDYFM |
| 20295 | EGNSNWVFQ |
| 20296 | EGPQNELNV |
| 20297 | EGVGLYEFA |
| 20298 | EHDNQDISV |
| 20299 | EHDTQAISV |
| 20300 | EHDTQDIIV |
| 20301 | EHDTQDSSV |
| 20302 | EHHALGVMG |
| 20303 | EHHDCVPWS |
| 20304 | EHHHSFGCP |
| 20305 | EHHNLGYHK |
| 20306 | EHHNVGYHT |
| 20307 | EHHPLGAMG |
| 20308 | EHHPVGVMG |
| 20309 | EHHTLGVMG |
| 20310 | EHLNLGYHT |
| 20311 | EHRNLGYHT |
| 20312 | EHVRGMEER |
| 20313 | EHVRGMVDR |
| 20314 | EHYTQDISV |
| 20315 | EIAHKLDDI |
| 20316 | EIALKLDDL |
| 20317 | EINEDYAKI |
| 20318 | EKDQRGIGS |
| 20319 | EKRCHAIAL |
| 20320 | ELAGKAFDH |
| 20321 | ELYVILIWW |
| 20322 | EMPIEWDEQ |
| 20323 | EMRFNNFCY |
| 20324 | ENDTQDISV |
| 20325 | ENTMSIIEH |
| 20326 | ENTMTIIEH |
| 20327 | EPIKIFNDA |
| 20328 | EPIQIFDDA |
| 20329 | EPIQIFNDS |
| 20330 | EPIQIFSDA |
| 20331 | EPIQIVNDA |
| 20332 | EPIQMFNDA |
| 20333 | EPIQSFNDA |
| 20334 | EPIWKVEDW |
| 20335 | EPLQIFNDA |
| 20336 | EPNIHAKCV |
| 20337 | EPPLSPSAS |
| 20338 | EPVFGFWNY |
| 20339 | EQHNLGYHT |
| 20340 | EQIASYDRV |
| 20341 | ERDTQDISV |
| 20342 | ESCEDEGLL |
| 20343 | ESCEDEGML |
| 20344 | ESCEDEGTL |
| 20345 | ESCEDEGVL |
| 20346 | ESCEDGGIL |
| 20347 | ESCEDQGIL |
| 20348 | ESCQDEGIL |
| 20349 | ESKYVFPNQ |
| 20350 | ESKYVIPNL |
| 20351 | ESKYVIPYQ |
| 20352 | ESKYVISNQ |
| 20353 | ESKYVVPNQ |
| 20354 | ESMERWGYD |
| 20355 | ESTNDNITI |
| 20356 | ESTTDNIAI |
| 20357 | ETCGPGGYT |
| 20358 | ETHYNEFCK |
| 20359 | ETRCYKYWN |
| 20360 | ETVETHRDR |
| 20361 | EVGGVRIGL |
| 20362 | EVKRWMMAD |
| 20363 | EVKRWMMDD |
| 20364 | EVKRWMMGD |
| 20365 | EVKRWMMVG |
| 20366 | EVKRWMTVD |
| 20367 | EVPKNELNV |
| 20368 | EVQRWMMVD |
| 20369 | EVRRWMMVD |
| 20370 | EWAQPMIMD |
| 20371 | EWIQPMIMD |
| 20372 | EWTQPLIMD |
| 20373 | EWTQPMIMG |
| 20374 | FATPKEGFD |
| 20375 | FCDGCAPED |
| 20376 | FCDRCAAED |
| 20377 | FCDRCAPEG |
| 20378 | FCDRCAPEV |
| 20379 | FCDRCAPEY |
| 20380 | FCDRCAPKD |
| 20381 | FCDRCATED |
| 20382 | FCDRCDPED |
| 20383 | FCDRCTPED |
| 20384 | FCGRCAPED |
| 20385 | FFFFPENED |
| 20386 | FFICPENED |
| 20387 | FFIFAENED |
| 20388 | FFIFHENED |
| 20389 | FFIFLENED |
| 20390 | FFIFPEDED |
| 20391 | FFIFPEHED |
| 20392 | FFIFPEIED |
| 20393 | FFIFPENAD |
| 20394 | FFIFPENEA |
| 20395 | FFIFPENEV |
| 20396 | FFIFPENGD |
| 20397 | FFIFPENQD |
| 20398 | FFIFPENVD |
| 20399 | FFIFPETED |
| 20400 | FFIFPEYED |
| 20401 | FFIFRENED |
| 20402 | FFIFSENED |
| 20403 | FFIFTENED |
| 20404 | FFIIPENED |
| 20405 | FFIVPENED |
| 20406 | FFIYPENED |
| 20407 | FFLFPENED |
| 20408 | FFNINGSVE |
| 20409 | FFVFPENED |
| 20410 | FGLKNDDRK |
| 20411 | FHALWAECF |
| 20412 | FIDAKIWDV |
| 20413 | FIIFPENED |
| 20414 | FIYKCLFGF |
| 20415 | FLVFMIIEG |
| 20416 | FLVFMLIEV |
| 20417 | FNGNDMLMV |
| 20418 | FPEHKDQGI |
| 20419 | FQEHKDKGI |
| 20420 | FQEHKDQGV |
| 20421 | FTNAAPVQD |
| 20422 | FWESDRVMM |
| 20423 | FWKSDRVMV |
| 20424 | FWKTDRVMM |
| 20425 | FYGPNWEFH |
| 20426 | FYIFPENED |
| 20427 | FYLPNWEFH |
| 20428 | FYQQFFEGI |
| 20429 | FYREFFEGI |
| 20430 | FYRKFFEGI |
| 20431 | FYRPFFEGI |
| 20432 | FYRPNWECH |
| 20433 | FYRPNWEFD |
| 20434 | FYRPNWEFL |
| 20435 | FYRPNWEFY |
| 20436 | FYRPNWEIH |
| 20437 | FYRPNWEVH |
| 20438 | FYRQFFAGI |
| 20439 | FYRQFFEGF |
| 20440 | FYRQFFEGL |
| 20441 | FYRQFFEGV |
| 20442 | FYRQFFQGI |
| 20443 | FYRQYFEGI |
| 20444 | GARKNGFMQ |
| 20445 | GARMNGFMR |
| 20446 | GARMNGFTQ |
| 20447 | GARTNGFMQ |
| 20448 | GCQQDDTDI |
| 20449 | GDCACKFSA |
| 20450 | GDCECKFAA |
| 20451 | GDCECKFSS |
| 20452 | GDCECTFSA |
| 20453 | GDCGCKFSA |
| 20454 | GDHLGNVWY |
| 20455 | GDHWHGPGD |
| 20456 | GDIPHRVAC |
| 20457 | GDMWWYQDP |
| 20458 | GDPADRNTC |
| 20459 | GDPGDRNTC |
| 20460 | GDPLDRNTC |
| 20461 | GDPVDHNTC |
| 20462 | GDPVDRNAC |
| 20463 | GDRLDREVF |
| 20464 | GDYCTHKNH |
| 20465 | GDYECKFSA |
| 20466 | GDYLGNIWY |
| 20467 | GEAQQRVVK |
| 20468 | GECECKFSA |
| 20469 | GEFGPNHRH |
| 20470 | GEFMEQGCG |
| 20471 | GEFWNWSRP |
| 20472 | GEFWTWSRT |
| 20473 | GEIPDRVAC |
| 20474 | GEIPHRVSC |
| 20475 | GEIPNRVAC |
| 20476 | GEIPPRVAC |
| 20477 | GEPVDRNTC |
| 20478 | GEWSWAEHS |
| 20479 | GFLHVTSIM |
| 20480 | GFPDEGSLQ |
| 20481 | GFPDEKSLQ |
| 20482 | GFPDERNLQ |
| 20483 | GFPDERSLE |
| 20484 | GFPDERSLK |
| 20485 | GFTDERSLQ |
| 20486 | GLELGHVWG |
| 20487 | GPFHQDARA |
| 20488 | GPFKQDARA |
| 20489 | GPFLQDARA |
| 20490 | GPFQEDARA |
| 20491 | GPFQQDARS |
| 20492 | GPFQQDDRA |
| 20493 | GPFQQDGRA |
| 20494 | GPVQQDARA |
| 20495 | GSISNVKAN |
| 20496 | GSQCGVPND |
| 20497 | GTFSNVKAN |
| 20498 | GTISNVKDN |
| 20499 | GTISNVKPN |
| 20500 | GTISNVKSN |
| 20501 | GTISNVKTN |
| 20502 | GTISNVNAN |
| 20503 | GTISNVRAN |
| 20504 | GTKPDTFHG |
| 20505 | GTVSNVKAN |
| 20506 | GVDMCGLAE |
| 20507 | GVISCPDCI |
| 20508 | GVISCPECL |
| 20509 | GYAGVESLG |
| 20510 | GYAGVGILG |
| 20511 | HANQDPWDG |
| 20512 | HATLDPWDG |
| 20513 | HFCANRHIA |
| 20514 | HFCDNRHIA |
| 20515 | HFCFNRHIA |
| 20516 | HFCLNRHIA |
| 20517 | HFCVNRHIE |
| 20518 | HFCVNRHNA |
| 20519 | HFCVNRNIA |
| 20520 | HFCVNRPIA |
| 20521 | HFFVNRHIA |
| 20522 | HFGVNRHIA |
| 20523 | HGLDLGCAV |
| 20524 | HGLKNDDRK |
| 20525 | HGTDQTMHM |
| 20526 | HIRLWPFEG |
| 20527 | HPMDDACKQ |
| 20528 | HSANRDHFV |
| 20529 | HSEDRDHFV |
| 20530 | HSEHRDHFV |
| 20531 | HSENRDHFG |
| 20532 | HSENRDHIV |
| 20533 | HSENRDPFV |
| 20534 | HSENRDYFV |
| 20535 | HSGNRDHFV |
| 20536 | HSKNRDHFV |
| 20537 | HTHYNEFCK |
| 20538 | HTKYECYRF |
| 20539 | HTSLLHFDH |
| 20540 | HVCVNRHIA |
| 20541 | HWPTHWNIP |
| 20542 | HWWFRKRWV |
| 20543 | HWWYRKRWE |
| 20544 | HYTKSISHS |
| 20545 | IDMWWYQDP |
| 20546 | IFDTRWSNV |
| 20547 | IFNYSNHND |
| 20548 | IFPYSNHND |
| 20549 | IINCNGMNW |
| 20550 | IINRTGMNW |
| 20551 | IIRLWPFEG |
| 20552 | ILGQWISYV |
| 20553 | IPDWLWKHV |
| 20554 | IPYKNQNNF |
| 20555 | ISEWNFQDW |
| 20556 | IVCQNNANS |
| 20557 | IYRPNWEFH |
| 20558 | KDHNREHII |
| 20559 | KDNECVSHT |
| 20560 | KDPETYTQM |
| 20561 | KEPEAYTQM |
| 20562 | KEPENYTQM |
| 20563 | KEPETYIQM |
| 20564 | KEPETYTKM |
| 20565 | KEPGTYTQM |
| 20566 | KEPKTYTQM |
| 20567 | KERETYTQM |
| 20568 | KESETYTQM |
| 20569 | KETETYTQM |
| 20570 | KGIDMAMEL |
| 20571 | KILTDIFDS |
| 20572 | KIWNFTKMM |
| 20573 | KIWNVTKVM |
| 20574 | KIWSVTKMM |
| 20575 | KKPETYTQM |
| 20576 | KMQCLTFCD |
| 20577 | KNIDYLKWN |
| 20578 | KNNEYLKWN |
| 20579 | KPHHWNKKT |
| 20580 | KPPHWNKET |
| 20581 | KPPHWNNKT |
| 20582 | KQPETYTQM |
| 20583 | KRNEDVLEQ |
| 20584 | KSMERWAYD |
| 20585 | KSMGRWGYD |
| 20586 | KTHYNEFCK |
| 20587 | KTIWGWSDC |
| 20588 | KTWNVTKMM |
| 20589 | KYISEWMEL |
| 20590 | LAMDCRPEH |
| 20591 | LDDPDTSGQ |
| 20592 | LFCVNRHIA |
| 20593 | LGICCYRDP |
| 20594 | LGMDWGMTD |
| 20595 | LGSLNGAMA |
| 20596 | LGSSNGAMD |
| 20597 | LGSSNGAVA |
| 20598 | LHCALGQHS |
| 20599 | LHCELGEHS |
| 20600 | LHCELGQHP |
| 20601 | LHCGLGQHS |
| 20602 | LHCKLGQHS |
| 20603 | LHCVLGQHS |
| 20604 | LHGKVSMHR |
| 20605 | LHWELGQHS |
| 20606 | LIFPNGDFQ |
| 20607 | LIPLWPFEG |
| 20608 | LIQLWPFEG |
| 20609 | LIRLWQFEG |
| 20610 | LIRLWRFEG |
| 20611 | LKMDCRPEH |
| 20612 | LLICCYRDP |
| 20613 | LLINDWISP |
| 20614 | LLVYFHVPN |
| 20615 | LNVADLPIN |
| 20616 | LNVDDMPIN |
| 20617 | LNVHDLPIN |
| 20618 | LNVVDLPIN |
| 20619 | LPADLVDDW |
| 20620 | LPADLVQDW |
| 20621 | LPADLVYDW |
| 20622 | LPDDLVHDW |
| 20623 | LPPHWIGHY |
| 20624 | LPSDLVHDW |
| 20625 | LPVDLVHDW |
| 20626 | LQMANRDPF |
| 20627 | LQMANRDSC |
| 20628 | LQMANRDTF |
| 20629 | LQMDNRDSF |
| 20630 | LQMPNRDSF |
| 20631 | LQMSNRDSF |
| 20632 | LQTHSICRD |
| 20633 | LQTKSICRD |
| 20634 | LRCELGQHS |
| 20635 | LRMDCRPEH |
| 20636 | LSEWNFHDW |
| 20637 | LSICCYRDP |
| 20638 | LSMDCRPEH |
| 20639 | LTMDCGPEH |
| 20640 | LTMDCRAEH |
| 20641 | LTMDCRPED |
| 20642 | LTMDCRPER |
| 20643 | LTMDCRPEY |
| 20644 | LTMDCRPGH |
| 20645 | LTMDCRPKH |
| 20646 | LTMDCRPKR |
| 20647 | LTMDGRPEH |
| 20648 | LTMGCRPEH |
| 20649 | LTMPNSEGK |
| 20650 | LTMPNSEQK |
| 20651 | LTPDWIGHY |
| 20652 | LTPHWIGHS |
| 20653 | LTPHWIVHY |
| 20654 | LWFCCYRDP |
| 20655 | LWICCYQDP |
| 20656 | LWICCYRDA |
| 20657 | LWICFYRDP |
| 20658 | LWMCCYRDP |
| 20659 | LWNCCYRDP |
| 20660 | LWVCCYRDP |
| 20661 | MAGINGMAY |
| 20662 | MAGLNGMAN |
| 20663 | MAGLNGMAS |
| 20664 | MAGLNGMEY |
| 20665 | MAGLNGVAY |
| 20666 | MAGVNGMAY |
| 20667 | MAHNGNWNR |
| 20668 | MDMSKAWMI |
| 20669 | MELGKDWIQ |
| 20670 | MFENWNLGV |
| 20671 | MFETKKRVV |
| 20672 | MGGLNGMAY |
| 20673 | MGIDKAMEL |
| 20674 | MGIDMAMEF |
| 20675 | MHCELGQHS |
| 20676 | MHDDNLNWN |
| 20677 | MHSPLIQYG |
| 20678 | MIEDRDYFI |
| 20679 | MIETKKRVI |
| 20680 | MISPLIQYG |
| 20681 | MISWWKVDF |
| 20682 | MKDAGEWLV |
| 20683 | MKFIQMIWV |
| 20684 | MLSALIQYG |
| 20685 | MLSHLIQYG |
| 20686 | MLSPLIQYC |
| 20687 | MLSTLIQYG |
| 20688 | MNFIQMILV |
| 20689 | MNFIQMLWV |
| 20690 | MNFISVHMC |
| 20691 | MNICVLDGP |
| 20692 | MNICVLDGT |
| 20693 | MNSAQTMVY |
| 20694 | MNSTQTMAY |
| 20695 | MNVDDLPIN |
| 20696 | MSHCLYVFG |
| 20697 | MTCNDMAFY |
| 20698 | MTIDSMAPC |
| 20699 | MTKWDRLHD |
| 20700 | MTKWDRWRD |
| 20701 | MWICCYRDP |
| 20702 | MWYLWWNCL |
| 20703 | MYLDCCDFK |
| 20704 | MYLDCRNFK |
| 20705 | MYMDCCNFK |
| 20706 | MYVDCCNFK |
| 20707 | NATEGLGWV |
| 20708 | NCVLFECCG |
| 20709 | NCVLYEFCG |
| 20710 | NCVVYECCG |
| 20711 | NEWSWAEHS |
| 20712 | NGARADAKK |
| 20713 | NGLDLGCGV |
| 20714 | NGTDQTMHM |
| 20715 | NGTRVDAKK |
| 20716 | NKVNWNHSS |
| 20717 | NNGFDRYID |
| 20718 | NRILDVEHD |
| 20719 | NSENRDHFV |
| 20720 | NTAEGLGWV |
| 20721 | NTIEGLGWV |
| 20722 | NTTEGLGWG |
| 20723 | NTTQGLGWV |
| 20724 | NVMEKYECR |
| 20725 | NVMEKYECS |
| 20726 | NWSAPQFSD |
| 20727 | NWSGPQFTD |
| 20728 | NWSTPQFTD |
| 20729 | PECCGIFSS |
| 20730 | PECCSICSS |
| 20731 | PECCSIFAS |
| 20732 | PECCSIFCS |
| 20733 | PECCSIFSA |
| 20734 | PECCSIFST |
| 20735 | PECCSIFYS |
| 20736 | PECCSIVSS |
| 20737 | PECCSIYSS |
| 20738 | PECCSLFSS |
| 20739 | PECCSSFSS |
| 20740 | PECCSTFSS |
| 20741 | PECCSVFSS |
| 20742 | PECCTIFSS |
| 20743 | PECWSIFSS |
| 20744 | PECYSIFSS |
| 20745 | PEELGYCYV |
| 20746 | PEELLYCYV |
| 20747 | PEELWYCHV |
| 20748 | PEEQWYCYV |
| 20749 | PEFCSIFSS |
| 20750 | PEKGMVEHQ |
| 20751 | PERENLERC |
| 20752 | PEWCSIFSS |
| 20753 | PFAHMESDA |
| 20754 | PFEHMASDA |
| 20755 | PFEHMDSDA |
| 20756 | PHQWSWSES |
| 20757 | PHRWSWIES |
| 20758 | PHRWSWSEP |
| 20759 | PILWTNHSN |
| 20760 | PIPWTNHSN |
| 20761 | PIQWTNHIN |
| 20762 | PIQWTNHNN |
| 20763 | PIQWTNPSN |
| 20764 | PISWCTQDL |
| 20765 | PISWCTQHV |
| 20766 | PLINQCREL |
| 20767 | PMLCNRGCE |
| 20768 | PMLCNRLCE |
| 20769 | PMLCNRVGE |
| 20770 | PMLWNRVCE |
| 20771 | PMMCNRVCE |
| 20772 | PPLGAWWYG |
| 20773 | PPPNTFHDS |
| 20774 | PQCCSIFSS |
| 20775 | PRNNDDWRT |
| 20776 | PRQLWEVWQ |
| 20777 | PSRDCDSWE |
| 20778 | PTIWGWSDC |
| 20779 | PTMDCRPEH |
| 20780 | PTRCLPLWT |
| 20781 | PVEWAYWHM |
| 20782 | QAMRCLFDH |
| 20783 | QARENLERC |
| 20784 | QECCSIFSS |
| 20785 | QEGENLERC |
| 20786 | QEPETYTQM |
| 20787 | QEPHPFSEQ |
| 20788 | QERENFERC |
| 20789 | QERENLDRC |
| 20790 | QERENVERC |
| 20791 | QIGCCMLMW |
| 20792 | QIIEFEERV |
| 20793 | QITCCMLMW |
| 20794 | QIWNVTKMM |
| 20795 | QLTCPLLDD |
| 20796 | QMLCNRVCE |
| 20797 | QMPMPPVAD |
| 20798 | QNHYNEFCK |
| 20799 | QNIEYLKWN |
| 20800 | QSDKNWYGM |
| 20801 | QSDQNWYSM |
| 20802 | QSKRCLFDH |
| 20803 | QTDYNEFCK |
| 20804 | QTHNNEFCK |
| 20805 | QTHYNEFCE |
| 20806 | QTHYNEFCR |
| 20807 | QTIGGWSDC |
| 20808 | QTIWGWGDC |
| 20809 | QTIWGWIDC |
| 20810 | QTIWGWNDC |
| 20811 | QTIWGWSDF |
| 20812 | QTIWVWSDC |
| 20813 | QTNYNEFCK |
| 20814 | QVCSDWCEG |
| 20815 | RAHECHQCD |
| 20816 | RAHVCDQCD |
| 20817 | RAHVCHHCD |
| 20818 | RAHVCHPCD |
| 20819 | RAHVCHQCA |
| 20820 | RAHVCNQCD |
| 20821 | RAHVCQQCD |
| 20822 | RARPHQGQQ |
| 20823 | RDQLDREVF |
| 20824 | RDRLDRAVF |
| 20825 | RDRLDREAF |
| 20826 | RDRLDREGF |
| 20827 | RDRLDRELF |
| 20828 | RDRLDREVC |
| 20829 | RDRLDREVS |
| 20830 | RDRLDREVV |
| 20831 | RDRLDREVY |
| 20832 | RDRLDRQVF |
| 20833 | RDRLDWEVF |
| 20834 | RDRLGREGF |
| 20835 | RDRLGREVF |
| 20836 | RDRMDREVF |
| 20837 | RDRRDREVF |
| 20838 | RDRSYEQMD |
| 20839 | RDRVDREVF |
| 20840 | RECIPMNSA |
| 20841 | REFWTWSRP |
| 20842 | REPETYTQM |
| 20843 | RERPQQGQQ |
| 20844 | RFCVNRHIA |
| 20845 | RFPDERSLQ |
| 20846 | RGGPQQGQQ |
| 20847 | RGHVCHQCD |
| 20848 | RGRPEQGQQ |
| 20849 | RGRPQEGQQ |
| 20850 | RGRPQQGEQ |
| 20851 | RGRPQQGKQ |
| 20852 | RGRPQQGQR |
| 20853 | RGRSQQGQQ |
| 20854 | RHFLAWSEI |
| 20855 | RHIDFFFGG |
| 20856 | RHNDYETHN |
| 20857 | RHRWSWSES |
| 20858 | RHSDYETHN |
| 20859 | RHTDYETHT |
| 20860 | RHWWGAACV |
| 20861 | RIDHNGGWF |
| 20862 | RIGHNGGGF |
| 20863 | RIRLWPFEG |
| 20864 | RIWNVTKMM |
| 20865 | RKPVEWHWE |
| 20866 | RLDLFIGRT |
| 20867 | RLGLFMGRT |
| 20868 | RLINDWIAP |
| 20869 | RLINDWIFP |
| 20870 | RLINDWIST |
| 20871 | RLNCLENHS |
| 20872 | RLNNDWISP |
| 20873 | RLNSLDNHS |
| 20874 | RLSNDWISP |
| 20875 | RLVNDWISP |
| 20876 | RMGSKEQED |
| 20877 | RMQCLTYCD |
| 20878 | RNPISVCSV |
| 20879 | RNPVDWHWE |
| 20880 | RNPVEWHWK |
| 20881 | RNPVEWNWE |
| 20882 | RPHVCHQCD |
| 20883 | RPWYHENDG |
| 20884 | RPWYHEYGG |
| 20885 | RRPNAHVDC |
| 20886 | RSDVCDNYD |
| 20887 | RSHVCHQCD |
| 20888 | RSMERWGYD |
| 20889 | RTDAQIGWQ |
| 20890 | RTDELIGWQ |
| 20891 | RTDEQIGWR |
| 20892 | RTDQQIGWQ |
| 20893 | RVGCLNVWG |
| 20894 | RVHVCHQCD |
| 20895 | RVMMDWQPD |
| 20896 | RWICCYRDP |
| 20897 | RYADMMSEY |
| 20898 | RYCLAWSEI |
| 20899 | RYFFAWSEI |
| 20900 | RYFLAWIEI |
| 20901 | RYFLAWSAI |
| 20902 | RYFLAWSES |
| 20903 | RYFLAWTEI |
| 20904 | RYFLDWSEI |
| 20905 | RYFSAWSEI |
| 20906 | RYFWAWSEI |
| 20907 | RYILAWSEI |
| 20908 | RYVLAWSEI |
| 20909 | SAPRLIEDR |
| 20910 | SCDRCAPED |
| 20911 | SCHTDDAKP |
| 20912 | SCKFDHMPP |
| 20913 | SCKLAHMPP |
| 20914 | SCKLDHMAP |
| 20915 | SCKLDHMSP |
| 20916 | SCKWDHMPP |
| 20917 | SCNPEDSIV |
| 20918 | SCNPEGSIG |
| 20919 | SDDGDHCTI |
| 20920 | SDDGYDIFG |
| 20921 | SDHGMHRDG |
| 20922 | SDMCWYQDP |
| 20923 | SDMGWYQDP |
| 20924 | SDMSWYQDP |
| 20925 | SDMWCYQDP |
| 20926 | SDMWGYQDP |
| 20927 | SDMWLYQDP |
| 20928 | SDMWWHQDP |
| 20929 | SDMWWYEDP |
| 20930 | SDMWWYKDP |
| 20931 | SDMWWYPDP |
| 20932 | SDMWWYQAP |
| 20933 | SDMWWYQDA |
| 20934 | SDMWWYQDH |
| 20935 | SDMWWYQDR |
| 20936 | SDMWWYQDT |
| 20937 | SDMWWYQHP |
| 20938 | SDMWWYQVP |
| 20939 | SDMWWYQYP |
| 20940 | SDMWWYRDP |
| 20941 | SDTWWYQDP |
| 20942 | SECCSIFSS |
| 20943 | SEGEHQCKA |
| 20944 | SEMWWYQDP |
| 20945 | SFWGPDVWG |
| 20946 | SGLKNDDRK |
| 20947 | SGMWWYQDP |
| 20948 | SMLCNRVCE |
| 20949 | SNCCSCKAN |
| 20950 | SNDCNLFDL |
| 20951 | SNDCSLCDL |
| 20952 | SNMWWYQDP |
| 20953 | SNNCNLCDL |
| 20954 | SPFQQDARA |
| 20955 | SSGSHDFMG |
| 20956 | SVFDNAFNN |
| 20957 | SVFDNGFDN |
| 20958 | SVLPDWGDS |
| 20959 | SVPRLIEDG |
| 20960 | SWKLDHMPP |
| 20961 | TCKLDHMPP |
| 20962 | TCNPEDSIG |
| 20963 | TCSELPERI |
| 20964 | TCVELPERI |
| 20965 | TDHNREHIS |
| 20966 | TDHNREHMI |
| 20967 | TDIDKQRDK |
| 20968 | TDVGDYWHA |
| 20969 | TDVMLAEWT |
| 20970 | TENDIGCAT |
| 20971 | TGGWMFFWH |
| 20972 | TGIDMAMEL |
| 20973 | TGLDFYRWF |
| 20974 | THNDIGCAT |
| 20975 | TKLNIRDRW |
| 20976 | TKTGNNVMH |
| 20977 | TKWNNLHIS |
| 20978 | TMLCNRVCE |
| 20979 | TMPMPPVAD |
| 20980 | TNCCSCKAN |
| 20981 | TNGFDKYID |
| 20982 | TNIDGLWPI |
| 20983 | TNNNDRFQV |
| 20984 | TNNVSEFMP |
| 20985 | TRLNDEDRG |
| 20986 | TSGSHDFMG |
| 20987 | TSMERWGYD |
| 20988 | TSPADHKWR |
| 20989 | TSSADHKWR |
| 20990 | TSTNDRFQV |
| 20991 | TTLNLRDRW |
| 20992 | TTMNIRDRW |
| 20993 | TTMRGFYHC |
| 20994 | TTTGNNVLH |
| 20995 | TTWNNLHII |
| 20996 | TYITKMDLV |
| 20997 | VCDRCAPED |
| 20998 | VEQLCIEWD |
| 20999 | VERLCIEWV |
| 21000 | VESNCIDDV |
| 21001 | VFQWINYYM |
| 21002 | VGTDQTMHM |
| 21003 | VHCELGQHS |
| 21004 | VHISNMEGD |
| 21005 | VLGQWIGYV |
| 21006 | VLGRWISYV |
| 21007 | VMEITNLWH |
| 21008 | VMGITHLWH |
| 21009 | VNCCSCKAN |
| 21010 | VNVHNWRCS |
| 21011 | VPHNFEFYT |
| 21012 | VSADKCNLN |
| 21013 | VSAGKCNVN |
| 21014 | VSCEDEGIL |
| 21015 | VSQNQNCFK |
| 21016 | VTKWDRWHD |
| 21017 | VWKSDRVMM |
| 21018 | VYRPNWEFH |
| 21019 | VYRQFFEGI |
| 21020 | VYTVNDGCV |
| 21021 | WEDSMFCQY |
| 21022 | WEHSMFCKY |
| 21023 | WEHSMFCRY |
| 21024 | WELSMFCQY |
| 21025 | WLYIWEKWP |
| 21026 | WLYTWEKWT |
| 21027 | WLYTWGKWP |
| 21028 | WLYTWVKWP |
| 21029 | WNPSNMFNI |
| 21030 | WRKAIPYVM |
| 21031 | WRKGITYVM |
| 21032 | WRKGTPYVM |
| 21033 | WRKGVPYVM |
| 21034 | WRKVIPYVM |
| 21035 | WRQGIPYVM |
| 21036 | WRRGIPYVM |
| 21037 | WVVNFIRQV |
| 21038 | WWIIEEHFK |
| 21039 | WWIIEEHGK |
| 21040 | WWVIEEHCK |
| 21041 | WYFLAWSEI |
| 21042 | YALKNDDRK |
| 21043 | YCHEYDEIV |
| 21044 | YCIFEYNGR |
| 21045 | YCIGCVDAI |
| 21046 | YCLKNDDRK |
| 21047 | YCNEFDEIV |
| 21048 | YCNEYDEII |
| 21049 | YCPAANWNW |
| 21050 | YCPEANWNL |
| 21051 | YCPEANWSW |
| 21052 | YCPEASWNW |
| 21053 | YCPETNWNW |
| 21054 | YCPGCGLMW |
| 21055 | YCPVANWNW |
| 21056 | YCREANWNW |
| 21057 | YCTEYDEIV |
| 21058 | YDHGMHRAG |
| 21059 | YDHGMHRDA |
| 21060 | YDHGMHRYG |
| 21061 | YDHGWKMAC |
| 21062 | YDIHPRFFH |
| 21063 | YDLCEHRWD |
| 21064 | YDPGMHRDG |
| 21065 | YEWSWAEHS |
| 21066 | YFCVNRHIA |
| 21067 | YFIFPENED |
| 21068 | YGIIMKIDL |
| 21069 | YGIRLKIDL |
| 21070 | YGIRNGKIG |
| 21071 | YGKISTIIN |
| 21072 | YGLENDDRK |
| 21073 | YGLKDDDRK |
| 21074 | YGLKNDARK |
| 21075 | YGLKNDDCK |
| 21076 | YGLKNDDRE |
| 21077 | YGLKNDDRR |
| 21078 | YGLKNDDSK |
| 21079 | YGLKNDGRK |
| 21080 | YGLKNDNRK |
| 21081 | YGLKNDYRK |
| 21082 | YGLMNDDRK |
| 21083 | YGLQNDDRK |
| 21084 | YGMKNDDRK |
| 21085 | YGMKNDDRR |
| 21086 | YGRKNDDRK |
| 21087 | YGVGYLMPA |
| 21088 | YGVKNDDRK |
| 21089 | YHLIAIGPS |
| 21090 | YHLIAIWPP |
| 21091 | YHLIAKWPS |
| 21092 | YHLIDIWPS |
| 21093 | YHPGCDCAC |
| 21094 | YLVGYLMPA |
| 21095 | YMKHFKNPS |
| 21096 | YMKHFRDPS |
| 21097 | YMKHFRNAS |
| 21098 | YMKHFRNSS |
| 21099 | YMKHIRNPS |
| 21100 | YMKHVRNPS |
| 21101 | YMKNFRNPS |
| 21102 | YMKQFRNPS |
| 21103 | YMKRFRNPS |
| 21104 | YMMHFRNPS |
| 21105 | YMRHFRNPS |
| 21106 | YNISIMEAG |
| 21107 | YQPGCDCSC |
| 21108 | YQPGCDCTC |
| 21109 | YSENRDHFV |
| 21110 | YSERKGEYG |
| 21111 | YTFGLRVDD |
| 21112 | YTNVDGFPI |
| 21113 | YTPGNNVTR |
| 21114 | YVFDNAFDN |
| 21115 | YVKISTVIN |
| 21116 | YVLKNDDRK |
| 21117 | YWNEYDEIV |

### Example 36

AAV5 Variants with Mammary Gland Tissue Tropism as Identified by Machine Learning

This example describes preferred properties of engineered AAV5 variants that display mammary gland tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display mammary gland tissue tropism.

Favored biophysical properties and favored amino acid residues at each position in the 581 to 589 region of an AAV5 VP capsid polypeptide, which are associated with a higher probability of mammary gland tissue tropism are described below. Any of the below machine learning-derived positional amino acid preferences described in TABLE 56 can be present alone or in combination with each other or in combination with any of the amino acid preferences observed and described in EXAMPLE 11, TABLE 21. Listed below in TABLE 56 are a summary of positional features shared between the top important features for mammary gland tropism extracted from the ML models.

TABLE 56
Machine Learning-Derived Mammary Gland Tissue Tropism Rules

Low surface accessibility at Xaa1
Xaa1 is selected from C
Medium mol mass at Xaa1
Xaa1 is selected from C
High surface accessibility at Xaa2
Xaa2 is selected from D, N, or Q
Low hydropathy at Xaa2
Xaa2 is selected from D, E, R, K, H, N, or Q
High average flexibility at Xaa3
Xaa3 is selected from D, E, R, P, G, or S
Medium mutability at Xaa3
Xaa3 is selected from R or H
High mutability at Xaa4
Xaa4 is selected from M, I, Q, or T
High solubility at Xaa4
Xaa4 is selected from W, F, I, or L
High surface accessibility at Xaa4
Xaa4 is selected from E, R, or K
High solubility at Xaa5
Xaa5 is selected from W, F, I, or L
Low mutability at Xaa5
Xaa5 is selected from Y, F, or L
High hydropathy at Xaa6
Xaa6 is selected from V, I, or L
Medium mol mass at Xaa6
Xaa6 is selected from D, I, L, or N
Low surface accessibility at Xaa8
Xaa8 is selected from C
Low mutability at Xaa8
Xaa8 is selected from C, R, or H
Medium mutability at Xaa9
Xaa9 is selected from R or H

TABLE 57 below provides sequences of the 581 to 589 region for variant AAV VP1 capsid polypeptides that exhibited mammary gland tissue tropism and comport to one or more of the rules provided in TABLE 56. The present disclosure, thus, provides for rAAVs composed of engineered AAV5 VP1 capsid polypeptides having a SEQ ID NO: 2, wherein the Xaa1 to Xaa9 region is any one of SEQ ID NO: 23118-SEQ ID NO: 24117, as disclosed in TABLE 57. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 57
Sequences of the 581 to 589 Region in AAV5
VP1 Capsid Polypeptide that Drive Mammary
Gland Tissue Tropism

| SEQ ID | 581-589 |
|---|---|
| NO | Sequence |
| 23118 | AAAQFGKHT |
| 23119 | AASKVKLHH |
| 23120 | ACDRFLAMY |
| 23121 | ACDRFMTMY |
| 23122 | ACDRFMVMY |
| 23123 | ACDRIMAMY |
| 23124 | ACDRVMAMY |
| 23125 | ACDRYIAEY |
| 23126 | ACDRYMAMY |
| 23127 | ADQCKWWCM |
| 23128 | ADQCQWWCV |
| 23129 | AELMALAPC |
| 23130 | AELMVLAQC |
| 23131 | AFDRFMAMY |
| 23132 | AFTIKDDWW |
| 23133 | AGAKYHDWL |
| 23134 | AGGKLKTIG |
| 23135 | AGGKLQTLV |
| 23136 | AIGQFMMHL |
| 23137 | AIQQFMMHL |
| 23138 | AIRLFMMHL |
| 23139 | AIRQFMMHR |
| 23140 | AIRQFMMHV |
| 23141 | AIRQFTMHL |
| 23142 | AIRRFMMHL |
| 23143 | AIWPAAWMD |
| 23144 | AKLMALAQC |
| 23145 | AKVNCVYYG |
| 23146 | AKVSTLNGM |
| 23147 | ALREFWTWW |
| 23148 | ALRQFMMHL |
| 23149 | ANCKHDHCH |
| 23150 | ANCKPDHCH |
| 23151 | ANCKQHHCH |
| 23152 | ANCRQDHCH |
| 23153 | ANRKQDHCH |
| 23154 | ANRQFMMHL |
| 23155 | ANWKQDHCH |
| 23156 | ANYKQDHCH |
| 23157 | AQISTLNGM |
| 23158 | AQNGLGTVQ |
| 23159 | ARGKLQTLG |
| 23160 | ARGPMLAVK |
| 23161 | ARVSTLNGM |
| 23162 | ASARLYYCW |
| 23163 | ASDIFDGHQ |
| 23164 | ASDKMDGGS |
| 23165 | ASDKMDIGS |
| 23166 | ASERLHYCW |
| 23167 | ASERLYYCL |
| 23168 | ASKQWDHKH |
| 23169 | ASNKMDVGS |
| 23170 | ASQRLYYCW |
| 23171 | ASRQFMMHL |
| 23172 | ASVRLYYCW |
| 23173 | ASYKDQRYY |
| 23174 | ATQKITGTS |
| 23175 | ATRQFMMHL |
| 23176 | ATTQWGMCN |
| 23177 | AVHPMAYCF |
| 23178 | AWDRFMAMY |
| 23179 | CAHKEHVCH |
| 23180 | CAHWSYDCY |
| 23181 | CAHWTYECY |
| 23182 | CAHWTYNCY |
| 23183 | CAHWTYYCY |
| 23184 | CAHWYYDQR |
| 23185 | CANKENVCH |
| 23186 | CANKGHVCH |
| 23187 | CANWTYDCY |
| 23188 | CATKEHVCH |
| 23189 | CEGWGTYCL |
| 23190 | CEGWVTYCI |
| 23191 | CFCILEKMT |
| 23192 | CFCTYEIEH |
| 23193 | CFCTYKIQH |
| 23194 | CFCTYKNEH |
| 23195 | CFGFVNSVT |
| 23196 | CFGTYKIEH |
| 23197 | CFGVVNSVN |
| 23198 | CFRTYDLLS |
| 23199 | CFWMYDLLS |
| 23200 | CFYTYKIEH |
| 23201 | CGHWTYDCY |
| 23202 | CGLIMEMCC |
| 23203 | CGLIMEMCL |
| 23204 | CGLIMEMWW |
| 23205 | CGLIMEVCW |
| 23206 | CGLIMQMCW |
| 23207 | CGLIMVMCW |
| 23208 | CGLLMEMCW |
| 23209 | CGMIMEMCW |
| 23210 | CGPIMEMCW |
| 23211 | CGVIMEMCW |
| 23212 | CHFQYKNQA |
| 23213 | CHFRYQNQA |
| 23214 | CHMSFNQCY |
| 23215 | CHRKWEKQE |
| 23216 | CIALRLAWD |
| 23217 | CICSSPDHP |
| 23218 | CICSSPEQP |
| 23219 | CIDLQLAWD |
| 23220 | CIDLRLAWG |
| 23221 | CIDLRLAWH |
| 23222 | CIDLRLDWD |
| 23223 | CIDLRLVWD |
| 23224 | CIDLWLAWD |
| 23225 | CIEWGGMMG |
| 23226 | CIEWWDMMG |
| 23227 | CIEWWGKMG |
| 23228 | CIEWWGMMA |
| 23229 | CIEWWGMVG |
| 23230 | CIEWWSMMG |
| 23231 | CIGAHWHIP |
| 23232 | CIGINLDHH |
| 23233 | CIGLRLAWD |
| 23234 | CIGLRLDWD |
| 23235 | CIGRFDHKQ |
| 23236 | CIGWWGMMG |
| 23237 | CIGYKWFPG |
| 23238 | CIHADGKYS |
| 23239 | CIIPKLKER |
| 23240 | CIIPKMEER |
| 23241 | CIIPKMKAR |
| 23242 | CIIPKMKEK |
| 23243 | CIIPKMKKR |
| 23244 | CIIPKMMER |
| 23245 | CIIPKMRER |
| 23246 | CIIPKVKER |
| 23247 | CIIPRMKER |
| 23248 | CIIRKMKER |
| 23249 | CIISKMKER |
| 23250 | CIKNDWQRI |
| 23251 | CIKPDWQRI |
| 23252 | CIKTDWERI |
| 23253 | CIKTDWQGI |
| 23254 | CIKTDWQRN |
| 23255 | CIKTDWRRI |
| 23256 | CILPKMKER |
| 23257 | CILSSPEQP |
| 23258 | CIMGHCRQH |
| 23259 | CIMPKMKER |
| 23260 | CIMSDCRQH |
| 23261 | CIMSHCREH |
| 23262 | CIMSHCRKH |
| 23263 | CIMSHCRQD |
| 23264 | CIMSHCRQY |
| 23265 | CIMSHFRQH |
| 23266 | CIMSHGRQH |
| 23267 | CIMSHNRQH |
| 23268 | CIMSHWRQH |
| 23269 | CIMSHYRQH |
| 23270 | CIMSRCRQH |
| 23271 | CIMTDWQRI |
| 23272 | CINADGKYP |
| 23273 | CINRFDHKQ |
| 23274 | CINSDGKYS |
| 23275 | CIPSDRHFG |
| 23276 | CIPTYRHLV |
| 23277 | CIQWWGMMG |
| 23278 | CIRSHCRQH |
| 23279 | CIRSSTDQP |
| 23280 | CIRTDWQRI |
| 23281 | CISRFDHKL |
| 23282 | CISRYDHKQ |
| 23283 | CITADGKYS |
| 23284 | CITTMNVKT |
| 23285 | CITTYSHFG |
| 23286 | CIVLRLAWD |
| 23287 | CIVPKMKER |
| 23288 | CIVWWGMMG |
| 23289 | CKHPSWYMD |
| 23290 | CKKWTNQAH |
| 23291 | CKPWTNQAH |
| 23292 | CKQWTNQAQ |
| 23293 | CKVHKHWDR |
| 23294 | CKVHRNWDR |
| 23295 | CKVRKNWDR |
| 23296 | CLAIFRLQS |
| 23297 | CLGATWVAA |
| 23298 | CLGSTWAAA |
| 23299 | CLGSTWFAA |
| 23300 | CLGSTWGAA |
| 23301 | CLGSTWVAT |
| 23302 | CMCLKKFYY |
| 23303 | CMCLKKYYY |
| 23304 | CMCLRKCYY |
| 23305 | CMCVKKCYY |
| 23306 | CMENYHQQY |
| 23307 | CMMSHCRQH |
| 23308 | CQDFRHDCA |
| 23309 | CQDFRHECS |
| 23310 | CQGKWEKQE |
| 23311 | CQKRIIMQA |
| 23312 | CQPKWEKQE |
| 23313 | CQQRLIMQA |
| 23314 | CQREWEKQE |
| 23315 | CQRKWEKQA |
| 23316 | CQRRWEKQE |
| 23317 | CRGKSHEHT |
| 23318 | CRGKSHNHT |
| 23319 | CRGKSRDHT |
| 23320 | CRGQSHDHT |
| 23321 | CRLIMEMCW |
| 23322 | CRRKSHDHT |
| 23323 | CSIWEDRSG |
| 23324 | CSMERRNEL |
| 23325 | CSMSHCRQH |
| 23326 | CSPNFQDQK |
| 23327 | CSRATWGAE |
| 23328 | CTDRMVKAA |
| 23329 | CTDRMVKAT |
| 23330 | CTGNYHQQY |
| 23331 | CTHKDIEMD |
| 23332 | CTHKQSIAV |
| 23333 | CTHRMVKAS |
| 23334 | CTLFSGRTA |
| 23335 | CTQSFVMYQ |
| 23336 | CTVFSGRAA |
| 23337 | CTVSSGRTA |
| 23338 | CVAIEMSVA |
| 23339 | CVCLKKCYY |
| 23340 | CVCTYKIEH |
| 23341 | CVFREIKTA |
| 23342 | CVGINLEHH |
| 23343 | CVGINLYHH |
| 23344 | CVGLNLDHH |
| 23345 | CVHGQYYQE |
| 23346 | CVLIMEMCW |
| 23347 | CVLYYNQQC |
| 23348 | CVMCYNDCK |
| 23349 | CVMSHCRQH |
| 23350 | CVQGQYNQE |
| 23351 | CVQGQYYQA |
| 23352 | CVQGRYYQE |
| 23353 | CVQTMNGRS |
| 23354 | CVVINLDHH |
| 23355 | CVYREIKTS |
| 23356 | CVYREIQTA |
| 23357 | CVYREITTA |
| 23358 | CVYRELKTA |
| 23359 | CVYREVKTA |
| 23360 | CWAIFRLRS |
| 23361 | CWAIFRMQS |
| 23362 | CWAIVRLQS |
| 23363 | CWAQLNTDP |
| 23364 | CWAQLNTDS |
| 23365 | CWDIFRLQS |
| 23366 | CWDIYNACD |
| 23367 | CWGIFRLQS |
| 23368 | CWGQLNTDT |
| 23369 | CWPQLNTDT |
| 23370 | CWSIFRLQS |
| 23371 | CWVMMKTQE |
| 23372 | CYCTYKIEH |
| 23373 | DAAKFGKHT |
| 23374 | DASKVKLHR |
| 23375 | DDLGAGRSE |
| 23376 | DDNWHAWFM |
| 23377 | DDVGAGRSD |
| 23378 | DDVMWQLIH |
| 23379 | DDWMPQWVD |
| 23380 | DEFIWLVMH |
| 23381 | DEISNQNSY |
| 23382 | DGGVTTYGG |
| 23383 | DHVIIKKNY |
| 23384 | DIRPSVSDS |
| 23385 | DKEWSVHMG |
| 23386 | DLDAQGAMW |
| 23387 | DLGMINNMA |
| 23388 | DMDAKGAMW |
| 23389 | DMDAQGSMW |
| 23390 | DMSIATWNP |
| 23391 | DPDKQGQGI |
| 23392 | DPKWEKYAM |
| 23393 | DPKWEKYGT |
| 23394 | DQAWSVHMG |
| 23395 | DQEWSVHMC |
| 23396 | DQEWSVNMG |
| 23397 | DQKWEKYGL |
| 23398 | DQKWEKYGM |
| 23399 | DQKWSVHMG |
| 23400 | DQVWSVHMG |
| 23401 | DREWSVHMG |
| 23402 | DSKWLDRPV |
| 23403 | DSSKVKLHH |
| 23404 | DTDKKGQGI |
| 23405 | DTDKQGQGF |
| 23406 | DTDKQGQGL |
| 23407 | DTDKQGQGM |
| 23408 | DTDKQGQGV |
| 23409 | DTDKQSQGI |
| 23410 | DTNKQGQGI |
| 23411 | DVDYRSKAW |
| 23412 | DVDYRYEAW |
| 23413 | DVFIDWSGR |
| 23414 | DVKTDTSRD |
| 23415 | DVRQILLCH |
| 23416 | DVRQILVCH |
| 23417 | DVRQIMMCH |
| 23418 | DVRQLLMCH |
| 23419 | DYFQYNEAR |
| 23420 | DYYQYNEAP |
| 23421 | EAISYVENV |
| 23422 | ECDIFDGHQ |
| 23423 | ECDILDGHQ |
| 23424 | ECDRYIAEH |
| 23425 | ECDRYIAQY |
| 23426 | ECDRYISEY |
| 23427 | ECGFIRLNE |
| 23428 | ECKPIAGDR |
| 23429 | ECYHVLANT |
| 23430 | EDIHMIAGH |
| 23431 | EDTHMIAGP |
| 23432 | EDTHMIVGH |
| 23433 | EEFDTIAAP |
| 23434 | EEFDTLAAP |
| 23435 | EEFDTVAAR |
| 23436 | EEFDTVATP |
| 23437 | EEFDTVGAP |
| 23438 | EEYDTVAAP |
| 23439 | EFDIDQCCT |
| 23440 | EGDIFDGHQ |
| 23441 | EGIWQGMDA |
| 23442 | EHVIIEKNY |
| 23443 | EHVILKKNY |
| 23444 | EHVIMKKNY |
| 23445 | EIKVVNSKD |
| 23446 | EIKYRRCRA |
| 23447 | EIKYRRFMA |
| 23448 | EIKYRRSMA |
| 23449 | EIREGEECC |
| 23450 | EIRVVNSRD |
| 23451 | EISPAAWMD |
| 23452 | EKMESPECL |
| 23453 | EKTWLVVWC |
| 23454 | EMDAQGAMW |
| 23455 | EMRMNIQCE |
| 23456 | EPEGALDKT |
| 23457 | EPEGELDRT |
| 23458 | EPEGELYKT |
| 23459 | EPEGEVDKT |
| 23460 | EQEAYILRI |
| 23461 | ESDIFDAHQ |
| 23462 | ESDIFDGHE |
| 23463 | ESDIFDGHK |
| 23464 | ESDIFDGHP |
| 23465 | ESDIFDGHR |
| 23466 | ESDIFDGRQ |
| 23467 | ESDIFDVHQ |
| 23468 | ESDIFHGHQ |
| 23469 | ESDIFVGHQ |
| 23470 | ESDISDGHQ |
| 23471 | ESDIVDGHP |
| 23472 | ESDIVDGHQ |
| 23473 | ESDPMQLFT |
| 23474 | ESDPMQRFT |
| 23475 | ESDVFEGHQ |
| 23476 | ESKSCGLKG |
| 23477 | ESKSCGLRG |
| 23478 | ESNIFDGHQ |
| 23479 | ESTSCGLTG |
| 23480 | ESVDWIKCE |
| 23481 | ESWPAAWMD |
| 23482 | ETDIFDGHQ |
| 23483 | EVEAIRGIR |
| 23484 | EVEIFRLEG |
| 23485 | EVIAYVENV |
| 23486 | FDWMPQWVD |
| 23487 | FGGQLRVMY |
| 23488 | FGRERVWDS |
| 23489 | FGSLYRRRG |
| 23490 | FIGLRYKDQ |
| 23491 | FKLWKLFQA |
| 23492 | FKMDMTKRS |
| 23493 | FKMWKLFKA |
| 23494 | FKRWKLFQA |
| 23495 | FNIPCLFSR |
| 23496 | FNIQCLCSR |
| 23497 | FNIQCLFTR |
| 23498 | FNIQCLISR |
| 23499 | FNIQCLYSR |
| 23500 | FNIRCLFSR |
| 23501 | FNLQCLFSR |
| 23502 | FQTSMYCDW |
| 23503 | FRGHAMWDA |
| 23504 | FRGHGMWYA |
| 23505 | FRGHGTWDA |
| 23506 | FRGHGVWDA |
| 23507 | FRGLGMWDA |
| 23508 | FSEQNVSEA |
| 23509 | FSIQCLFSR |
| 23510 | FTEQIGRQM |
| 23511 | FTQQIDRQM |
| 23512 | FVCLRYKDQ |
| 23513 | FVGWYLNSP |
| 23514 | FVGWYWDSP |
| 23515 | FVGWYWNPP |
| 23516 | FVGWYWNSH |
| 23517 | FVGWYWNSR |
| 23518 | FVMGYNDCK |
| 23519 | GCGLVQRFM |
| 23520 | GHFTVLNWG |
| 23521 | GNCKQDHCH |
| 23522 | GRHTAVATG |
| 23523 | GSERLYYCW |
| 23524 | GSVMVAITG |
| 23525 | GYGRMNKAS |
| 23526 | HASICQGGD |
| 23527 | HAYIYETWP |
| 23528 | HDCDMQASE |
| 23529 | HDKWGWWRE |
| 23530 | HDRKACMIA |
| 23531 | HDVMWQLIH |
| 23532 | HDWMPQWVD |
| 23533 | HEAKLVPKA |
| 23534 | HEFIWLEMH |
| 23535 | HEYIYETLP |
| 23536 | HEYIYVTWP |
| 23537 | HFDFNHQCQ |
| 23538 | HFLEVAWGS |
| 23539 | HHRHIANNG |
| 23540 | HKHMILYHF |
| 23541 | HMDIQMVMP |
| 23542 | HMDIQMYMP |
| 23543 | HMDIQVFMP |
| 23544 | HMSIATWYP |
| 23545 | HMTFYLSRD |
| 23546 | HPSICEGGD |
| 23547 | HSEMAMLMQ |
| 23548 | HSEMVMWMQ |
| 23549 | HSGELYDAF |
| 23550 | HSGELYDCF |
| 23551 | HSGELYDSF |
| 23552 | HSGIHCCCG |
| 23553 | HSNFFHLND |
| 23554 | HSNFFHMTD |
| 23555 | HVDGNIACN |
| 23556 | HVDSNIACT |
| 23557 | HVDSNISCN |
| 23558 | HVDSNLACN |
| 23559 | HVDSNVACN |
| 23560 | HVNSNIACN |
| 23561 | HYCKIIPCH |
| 23562 | HYCKIIPSH |
| 23563 | HYCKIIPYN |
| 23564 | HYCKIITYH |
| 23565 | HYCKILPYH |
| 23566 | HYCKLIPYH |
| 23567 | HYCKMIPYH |
| 23568 | HYCKNIPYH |
| 23569 | HYCKTIPYH |
| 23570 | HYCMIIPYH |
| 23571 | HYCQIIPYH |
| 23572 | HYCRIIPYH |
| 23573 | HYFKIIPYH |
| 23574 | HYGKIIPYH |
| 23575 | HYYKIIPYH |
| 23576 | ICKIRSRLD |
| 23577 | ICTIGSRLD |
| 23578 | IDRKACMIA |
| 23579 | IGVQFLKDH |
| 23580 | IIGDKTQDD |
| 23581 | IIRDKTEDD |
| 23582 | IIRDKTQDG |
| 23583 | IIRDKTQND |
| 23584 | IIRDKTRDD |
| 23585 | IIRDRTQDD |
| 23586 | IIRDTTQDD |
| 23587 | IIREKTQDD |
| 23588 | IIRGKTQDD |
| 23589 | IIRYKTQDD |
| 23590 | IMRDKTQDD |
| 23591 | IPRTYQEMQ |
| 23592 | IQHQSRRHM |
| 23593 | IQLKAVPSC |
| 23594 | IQLKAVTCC |
| 23595 | IQLKTVTSC |
| 23596 | IRGHGMWDA |
| 23597 | IRLKAVTSC |
| 23598 | IRRHQMTDK |
| 23599 | ISRDKTQDD |
| 23600 | ITRTYQEME |
| 23601 | ITRTYQEMK |
| 23602 | ITRTYQERQ |
| 23603 | IVRDKTQDD |
| 23604 | KANMWLACV |
| 23605 | KCGFIRLNE |
| 23606 | KCVIGKGRR |
| 23607 | KDFWEHECF |
| 23608 | KDFWEHLCF |
| 23609 | KDFWEHQCV |
| 23610 | KDFWERQCF |
| 23611 | KDFWEYQCF |
| 23612 | KDFWGHQCF |
| 23613 | KDFWKHQCF |
| 23614 | KDRMGINQC |
| 23615 | KDRMGLNQR |
| 23616 | KDRMGLNQW |
| 23617 | KDRMVLNQC |
| 23618 | KDTHMIAGH |
| 23619 | KDVTRLRCP |
| 23620 | KDVWEHQCF |
| 23621 | KEFDTVAAP |
| 23622 | KFDIDQCCT |
| 23623 | KKFWDTVIY |
| 23624 | KKHMILCHF |
| 23625 | KQTQIRRHM |
| 23626 | KSDIFDGHQ |
| 23627 | KSVDWIKCE |
| 23628 | KYAYILDCA |
| 23629 | KYAYSLYCA |
| 23630 | KYGYSLDCA |
| 23631 | KYTYSLDCA |
| 23632 | KYWNYAEAK |
| 23633 | LADIEYSHD |
| 23634 | LAGKEGAHF |
| 23635 | LCASRGLDA |
| 23636 | LCGAIIKAS |
| 23637 | LCGLIIKAS |
| 23638 | LCGPIIKAA |
| 23639 | LCGPIIKAP |
| 23640 | LCGPIIKAY |
| 23641 | LCGPIIKPS |
| 23642 | LCGPIIKSS |
| 23643 | LCGPIIRAS |
| 23644 | LCGPILKAS |
| 23645 | LCGPIVKAS |
| 23646 | LCGPLIKAS |
| 23647 | LCGQIIKAS |
| 23648 | LCTSRGLDP |
| 23649 | LDRKACLIA |
| 23650 | LDRKACMIG |
| 23651 | LDRKACMIS |
| 23652 | LDRKACMMA |
| 23653 | LDRKACMVA |
| 23654 | LDRKACVIA |
| 23655 | LDRKAFMIA |
| 23656 | LDRKAWMIA |
| 23657 | LDRKDCMIA |
| 23658 | LDRKPCMIA |
| 23659 | LDRKSCMIA |
| 23660 | LDRKVCMIA |
| 23661 | LDRQACMIA |
| 23662 | LDRRACMIA |
| 23663 | LFTDYWEQG |
| 23664 | LGILYRRRG |
| 23665 | LGSPYRRRG |
| 23666 | LIRDKTQDD |
| 23667 | LKAQFHWGA |
| 23668 | LKDQFHWCA |
| 23669 | LKDRFHWGA |
| 23670 | LKMDMTKHS |
| 23671 | LKVDMTKRS |
| 23672 | LLFFMKFAK |
| 23673 | LQMDMTKRS |
| 23674 | LQPQMRRHM |
| 23675 | LQPQNRRHM |
| 23676 | LQPQSRRHV |
| 23677 | LRCQMGDHW |
| 23678 | LRMDMTKRS |
| 23679 | LRVIQGDNY |
| 23680 | LTHKRESEW |
| 23681 | LTPKRESEC |
| 23682 | LTRKRESEC |
| 23683 | LWFYMKFAK |
| 23684 | MANKWLACV |
| 23685 | MANRWLACV |
| 23686 | MASMWLACV |
| 23687 | MATMWLACV |
| 23688 | MCGLRYYWA |
| 23689 | MCGPIIKAS |
| 23690 | MCLLIRACE |
| 23691 | MCLWILACE |
| 23692 | MCLWIRPCE |
| 23693 | MCRHIIPDN |
| 23694 | MEHQSRRHM |
| 23695 | MEPESRRHM |
| 23696 | MEPQSRRHI |
| 23697 | MEPQSRRHM |
| 23698 | MFEKYFQSM |
| 23699 | MFMKDELIA |
| 23700 | MFMKNELMA |
| 23701 | MHKHFEDHK |
| 23702 | MHKHFKGHK |
| 23703 | MHKHYKDHK |
| 23704 | MHKRFKDHK |
| 23705 | MHLLKDFCT |
| 23706 | MHQHFKDHK |
| 23707 | MHRHFKDHK |
| 23708 | MIDWGNKCT |
| 23709 | MKPQGRRHM |
| 23710 | MKPQSRRHL |
| 23711 | MLAGWWQDT |
| 23712 | MLGEWWPGC |
| 23713 | MLNWGNKCT |
| 23714 | MNKHFKDHK |
| 23715 | MPKHFKDHK |
| 23716 | MQHLSRRHM |
| 23717 | MQHQCRRHM |
| 23718 | MQHRSRRHM |
| 23719 | MQLQCRRHM |
| 23720 | MQLRSRRHM |
| 23721 | MQPDSRRHM |
| 23722 | MQPHIRRHM |
| 23723 | MQPHSRRDM |
| 23724 | MQPLSRRHM |
| 23725 | MQPPSRRRM |
| 23726 | MQPQFRRHM |
| 23727 | MQPQSRRDM |
| 23728 | MQPQSRRHK |
| 23729 | MQPQSRRNM |
| 23730 | MQPRGRRHM |
| 23731 | MQPRSRRHM |
| 23732 | MQRRSRRHM |
| 23733 | MQTESRRHM |
| 23734 | MQTQSLRHM |
| 23735 | MQTQSRRNM |
| 23736 | MRIIQGDNY |
| 23737 | MRPQCRRHM |
| 23738 | MRPQSRRHK |
| 23739 | MRPQSRRHM |
| 23740 | MRPQSRRHR |
| 23741 | MRVIQGDNC |
| 23742 | MSNMWLACV |
| 23743 | MSPKPAYWG |
| 23744 | MSTKPAYWA |
| 23745 | MSTKPEYWG |
| 23746 | MTAKMAGRM |
| 23747 | MTNMWLACV |
| 23748 | MVAIKQYHT |
| 23749 | MVGVTRDAS |
| 23750 | MVVIKQYQT |
| 23751 | MVVIKRYHT |
| 23752 | MWFFMKFAK |
| 23753 | MWGEWWPGG |
| 23754 | MWVIQGDNY |
| 23755 | MYKHFKDHK |
| 23756 | NDGMVHNCW |
| 23757 | NDHWHAWFM |
| 23758 | NDKWGWWRQ |
| 23759 | NDLMWQLIH |
| 23760 | NDNWDAWFM |
| 23761 | NDNWHAWFR |
| 23762 | NDNWHAWFV |
| 23763 | NDNWHAWIM |
| 23764 | NDNWHEWFM |
| 23765 | NDVMWELIH |
| 23766 | NDVMWHLIH |
| 23767 | NDVMWQLIR |
| 23768 | NDVMWQLIY |
| 23769 | NDVMWQLVH |
| 23770 | NDWMPQWVD |
| 23771 | NEISNQNTY |
| 23772 | NFDFNHQCH |
| 23773 | NFGIYHLQG |
| 23774 | NKHCIAMPC |
| 23775 | NKYCIAMAC |
| 23776 | NKYCIAMPF |
| 23777 | NKYCITMPC |
| 23778 | NKYCLAMPC |
| 23779 | NMDAQGAMW |
| 23780 | NNGEWDVCY |
| 23781 | NPKWEKYGM |
| 23782 | NQDWLLSVP |
| 23783 | NQEWSVHMG |
| 23784 | NVDVRESEW |
| 23785 | NWHEKRHRG |
| 23786 | NYKPSFLTR |
| 23787 | PCAIEESHD |
| 23788 | PCDRFMAMY |
| 23789 | PEDFENHDG |
| 23790 | PLRAEMSFH |
| 23791 | QCDRVWLNQ |
| 23792 | QCEFIRLNE |
| 23793 | QCGFIRLDG |
| 23794 | QCGFIRLHE |
| 23795 | QCGFIRLNG |
| 23796 | QCGFIRLNQ |
| 23797 | QCGFIRVNE |
| 23798 | QCGFLRLNE |
| 23799 | QCHIIVAPE |
| 23800 | QCHIMVARE |
| 23801 | QCHINVARE |
| 23802 | QCPIIVARE |
| 23803 | QDFWEHQCF |
| 23804 | QEFDTVAAP |
| 23805 | QEHMILCHF |
| 23806 | QETWLVVWC |
| 23807 | QFGFIRLNE |
| 23808 | QFHIDQCCT |
| 23809 | QGGFIRLNA |
| 23810 | QGGFIRLNG |
| 23811 | QIGCNVMIS |
| 23812 | QIGFNVMLS |
| 23813 | QIKYRRCMA |
| 23814 | QIWPAAWMD |
| 23815 | QKDMIICHF |
| 23816 | QKDMILCHF |
| 23817 | QKDWGRRVT |
| 23818 | QKEAYILRI |
| 23819 | QKHMIICHF |
| 23820 | QKHMILCGF |
| 23821 | QKHMILCRF |
| 23822 | QKHMILCYF |
| 23823 | QKHMLLCHF |
| 23824 | QKHMMLCHF |
| 23825 | QKHMSLCHF |
| 23826 | QKHMTLCHF |
| 23827 | QKHRILCHF |
| 23828 | QKHRLLCHF |
| 23829 | QKHTILCHF |
| 23830 | QKHVILCHF |
| 23831 | QKIWLVVWC |
| 23832 | QKKMILCHF |
| 23833 | QKNMILCHF |
| 23834 | QKPMILCHF |
| 23835 | QKPWLVVWC |
| 23836 | QKRMILCHF |
| 23837 | QKTWLIVWC |
| 23838 | QKTWLVGWC |
| 23839 | QKTWLVVWF |
| 23840 | QKTWLVVWV |
| 23841 | QKTWLVVWY |
| 23842 | QNYWCVCGA |
| 23843 | QPDPMKQWS |
| 23844 | QPKEWVIKD |
| 23845 | QPREWVIKE |
| 23846 | QQDPMKQWT |
| 23847 | QQEACILRI |
| 23848 | QQEAFILRI |
| 23849 | QQEAYILKI |
| 23850 | QQEAYILRL |
| 23851 | QQEAYILRV |
| 23852 | QQEEYILRI |
| 23853 | QQEGYILRI |
| 23854 | QQESYILRI |
| 23855 | QQEVYILRI |
| 23856 | QQHMILCHF |
| 23857 | QSDDWIKCE |
| 23858 | QSDIFDGHQ |
| 23859 | QSDRVWLNP |
| 23860 | QSEMAMWMQ |
| 23861 | QSGWLLEAK |
| 23862 | QSIDWIKCE |
| 23863 | QSLDWIKCE |
| 23864 | QSVDWIKGE |
| 23865 | QSVDWIKYE |
| 23866 | QSVDWIQCE |
| 23867 | QSVDWIRCE |
| 23868 | QSVDWLKCE |
| 23869 | QSVDWMKCE |
| 23870 | QSVDWVKCE |
| 23871 | QSVVWIKCE |
| 23872 | QSVYWIKCE |
| 23873 | QVKYRTCDW |
| 23874 | QVTSDGKIV |
| 23875 | QYSRMNKAS |
| 23876 | RAKTCIYKY |
| 23877 | RAKTCLYKC |
| 23878 | RCDGLWCHF |
| 23879 | RCGFIRLNE |
| 23880 | RCGFIRLNG |
| 23881 | RCMKLVGTA |
| 23882 | RCRNCGSNG |
| 23883 | RDFWEHQCF |
| 23884 | RDQCVASCA |
| 23885 | REAKVFEIK |
| 23886 | REAKVFEPK |
| 23887 | REPKVFELK |
| 23888 | RESKVFELK |
| 23889 | RETKVFELK |
| 23890 | RFKLLMTGR |
| 23891 | RKTWLVVWC |
| 23892 | RLKPSAVQR |
| 23893 | RLRNCGSNG |
| 23894 | RLVRDSKNT |
| 23895 | RLVRDSKTN |
| 23896 | RMIQPEHVV |
| 23897 | RMKWYLLCV |
| 23898 | RMKWYLTCV |
| 23899 | RMPIELMHG |
| 23900 | RNNWKVDSQ |
| 23901 | RNNWKVNSR |
| 23902 | RNYWCMCGA |
| 23903 | RPATVHASF |
| 23904 | RPEMVHASF |
| 23905 | RPERVHASF |
| 23906 | RQEAYILRI |
| 23907 | RQGCLLAWQ |
| 23908 | RQPQSRRHM |
| 23909 | RRDELLIPD |
| 23910 | RRIELLIPD |
| 23911 | RRKWYLPCV |
| 23912 | RRNELLLPD |
| 23913 | RRSELLIPD |
| 23914 | RRTELLIPD |
| 23915 | RSVDWIKCE |
| 23916 | RVADLMYIG |
| 23917 | RVGDVMYIG |
| 23918 | RVSDVMYIG |
| 23919 | RVTDVMYIG |
| 23920 | RVVRDSKNN |
| 23921 | RWGFIRLNE |
| 23922 | RWRHCGSNG |
| 23923 | RWRICGSNG |
| 23924 | RWRNCGGNG |
| 23925 | RWRNCGNNG |
| 23926 | RWRNCGSNA |
| 23927 | RWRNCSSNG |
| 23928 | RWRNGGSNG |
| 23929 | RWRSCGSNG |
| 23930 | RYCKIIPYH |
| 23931 | RYWNYAEAK |
| 23932 | SAGIFYAHK |
| 23933 | SAYIFYAHK |
| 23934 | SCGLVERFM |
| 23935 | SDQCQWWCM |
| 23936 | SGGLVQRFM |
| 23937 | SGNIYESGQ |
| 23938 | SIRDKTQDD |
| 23939 | SIRQFMMHL |
| 23940 | SIRQYMMHL |
| 23941 | SKGCIIAMQ |
| 23942 | SQVSTLNGM |
| 23943 | SSHAANFCG |
| 23944 | SSHATTFCG |
| 23945 | SSKYEMKFL |
| 23946 | STDKDIEMD |
| 23947 | STHKDIEMG |
| 23948 | STHKDIEMN |
| 23949 | STHKDIVMD |
| 23950 | STTQWGVCN |
| 23951 | SWHEKRHRG |
| 23952 | SWSMQSKST |
| 23953 | SYGQIEGYQ |
| 23954 | TCAVFVIHC |
| 23955 | TCDRFMAMY |
| 23956 | TCFIGKGRR |
| 23957 | TCGIGKGRR |
| 23958 | TCIIGKGRR |
| 23959 | TCIIGQGRR |
| 23960 | TCLIGKGRR |
| 23961 | TCVIAKGRR |
| 23962 | TCVIEKGRR |
| 23963 | TCVIGKGQR |
| 23964 | TCVIGQGRR |
| 23965 | TCVIGRGRR |
| 23966 | TCVIVKGRR |
| 23967 | TEDFENHDA |
| 23968 | TIRQFMMHL |
| 23969 | TKGCIIDMQ |
| 23970 | TKLNCVYYG |
| 23971 | TKVNCIYYG |
| 23972 | TLRAAMSFH |
| 23973 | TLRAEMSFR |
| 23974 | TLRAEMSIH |
| 23975 | TLRAVMSFH |
| 23976 | TNCKQDHCH |
| 23977 | TRVNCVYYG |
| 23978 | TSERLYYCW |
| 23979 | TSHATNFCG |
| 23980 | TSNKDQRYY |
| 23981 | TSSKDQRYY |
| 23982 | TSYKDERYY |
| 23983 | TSYKNQRYY |
| 23984 | TTTQWGMCN |
| 23985 | TVLAWGLCN |
| 23986 | TVRADMSFH |
| 23987 | TYKPSFWTR |
| 23988 | TYWNYAESK |
| 23989 | VASKTLWET |
| 23990 | VCFKLFHVG |
| 23991 | VDRKACMIA |
| 23992 | VFTDYWEKG |
| 23993 | VFTDYWERG |
| 23994 | VFVGKNAAW |
| 23995 | VGSLYRRRG |
| 23996 | VIRDKTQDD |
| 23997 | VLDAIHWHW |
| 23998 | VQPESRRHM |
| 23999 | VRFTVLNWG |
| 24000 | VRIQPQHNK |
| 24001 | VRLKPQHNK |
| 24002 | VRLQPKHNK |
| 24003 | VRLQPQHDK |
| 24004 | VRLQPQHDR |
| 24005 | VRLQPQHSR |
| 24006 | VRLQPQLNK |
| 24007 | VRLQPQRNK |
| 24008 | VSDPMQQFT |
| 24009 | VVHQMAYCF |
| 24010 | VWDWEHKCT |
| 24011 | VWFFMKFAK |
| 24012 | VWNGEHKCT |
| 24013 | WAAIEYSHD |
| 24014 | WADIDYSHD |
| 24015 | WADIEYGHD |
| 24016 | WADIEYSHA |
| 24017 | WAGIEYSHD |
| 24018 | WAYIEYSHD |
| 24019 | WCGPIIKAS |
| 24020 | WDFHSIFPG |
| 24021 | WDYHIIFPG |
| 24022 | WDYHSICPG |
| 24023 | WDYHSIFAG |
| 24024 | WDYHSIFPC |
| 24025 | WDYHSIYPG |
| 24026 | WDYHSVFPG |
| 24027 | WDYHTIFPG |
| 24028 | WQHGREADH |
| 24029 | WSNFALWCQ |
| 24030 | WSSFAVWCQ |
| 24031 | WSSFTLWCQ |
| 24032 | YAQSFSQGI |
| 24033 | YAWMPQWVD |
| 24034 | YCFINSAYP |
| 24035 | YCPRLVEAS |
| 24036 | YDCDMKASE |
| 24037 | YDCDMQASG |
| 24038 | YDCDVQASE |
| 24039 | YDCVMQASE |
| 24040 | YDFDMQASE |
| 24041 | YDGMPQWVD |
| 24042 | YDLMPKWVD |
| 24043 | YDLMPQWVD |
| 24044 | YDWDMQASE |
| 24045 | YDWMAQWVD |
| 24046 | YDWMHQWID |
| 24047 | YDWMPEWVD |
| 24048 | YDWMPHWGD |
| 24049 | YDWMPHWVE |
| 24050 | YDWMPKWVD |
| 24051 | YDWMPPWVD |
| 24052 | YDWMPQLVD |
| 24053 | YDWMPQWAD |
| 24054 | YDWMPQWDA |
| 24055 | YDWMPQWDD |
| 24056 | YDWMPQWFD |
| 24057 | YDWMPQWGA |
| 24058 | YDWMPQWGD |
| 24059 | YDWMPQWID |
| 24060 | YDWMPQWIG |
| 24061 | YDWMPQWIV |
| 24062 | YDWMPQWLG |
| 24063 | YDWMPQWVA |
| 24064 | YDWMPQWVH |
| 24065 | YDWMPQWVV |
| 24066 | YDWMPQWVY |
| 24067 | YDWMRQWVD |
| 24068 | YDWMSQWVD |
| 24069 | YDWMTQWVD |
| 24070 | YDWRPQWVD |
| 24071 | YDWVPQWVD |
| 24072 | YDYDMQASE |
| 24073 | YEAPTILNP |
| 24074 | YEWMPQCVD |
| 24075 | YFCILDKMT |
| 24076 | YFCIMEKMT |
| 24077 | YFCIQEKMT |
| 24078 | YFCSWNTQE |
| 24079 | YFDFNHQCQ |
| 24080 | YGHEKRHRG |
| 24081 | YGLIMEMCW |
| 24082 | YGWMPQWVD |
| 24083 | YHGHIANNG |
| 24084 | YHMGFNQCY |
| 24085 | YHMNFNQCY |
| 24086 | YHMSFKQCY |
| 24087 | YHMTFNQCY |
| 24088 | YHRHIADNG |
| 24089 | YHRHTANNG |
| 24090 | YHRNIANNG |
| 24091 | YIRDGGSHD |
| 24092 | YIRDGVSHG |
| 24093 | YIRDGVSHH |
| 24094 | YIRVGGNNQ |
| 24095 | YIRVGVSHD |
| 24096 | YLHEKRHRG |
| 24097 | YLRSLKCGD |
| 24098 | YNWMPQWVD |
| 24099 | YQASIYCDW |
| 24100 | YQTSIYCDW |
| 24101 | YSDVLIYAN |
| 24102 | YSDVLVCAN |
| 24103 | YSDVMICAN |
| 24104 | YVDSNIACN |
| 24105 | YVLESNYCE |
| 24106 | YVRDGVSHD |
| 24107 | YVRQILMCH |
| 24108 | YVVESNYCQ |
| 24109 | YVWMPQWVD |
| 24110 | YWHEQRHRG |
| 24111 | YWHERRHRG |
| 24112 | YWREKRHRG |
| 24113 | YYCKIIPYH |
| 24114 | YYFQYNEAP |
| 24115 | YYKPSFWTR |
| 24116 | YYWHVSAFG |
| 24117 | YYWHVSTFW |

### Example 37

AAV5 Variants with Lung Tissue Tropism as Identified by Machine Learning

This example describes preferred properties of engineered AAV5 variants that display lung tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display lung tissue tropism.

Favored biophysical properties and favored amino acid residues at each position in the 581 to 589 region of an AAV5 VP capsid polypeptide, which are associated with a higher probability of lung tissue tropism are described below. Any of the below machine learning-derived positional amino acid preferences described in TABLE 58 can be present alone or in combination with each other or in combination with any of the amino acid preferences observed and described in EXAMPLE 12, TABLE 23. Listed below in TABLE 58 are a summary of positional features shared between the top important features for lung tropism extracted from the ML models.

TABLE 58
Machine Learning-Derived Lung Tissue Tropism Rules

High mutability at Xaa1
Xaa1 is selected from D, E, M, A, I, Q, or T
High mol mass at Xaa2
Xaa2 is selected from F
Low mutability at Xaa2
Xaa2 is selected from Y, F, or L
Low mutability at Xaa3
Xaa3 is selected from K, V, P, or H
Low hydropathy at Xaa3
Xaa3 is selected from K or R
Low mutability at Xaa4
Xaa4 is selected from K or P
High average flexibility at Xaa4
Xaa4 is selected from D, E, P, or S
Low average flexibility at Xaa5
Xaa5 is selected from W, M, or F
High solubility at Xaa5
Xaa5 is selected from W, F, I, or L
Medium mutability at Xaa6
Xaa6 is selected from R or H
High surface accessibility at Xaa6
Xaa6 is selected from T
Low mutability at Xaa7
Xaa7 is selected from C
High solubility at Xaa7
Xaa7 is selected from W, V, M, F, I, or L
High mutability at Xaa8
Xaa8 is selected from D, E, M, A, I, Q, or T
Low hydropathy at Xaa8
Xaa8 is selected from R or K
High average flexibility at Xaa9
Xaa9 is selected from R or G

TABLE 59 below provides sequences of the 581 to 589 region for variant AAV VP1 capsid polypeptides that exhibited lung tissue tropism and comport to one or more of the rules provided in TABLE 58. The present disclosure, thus, provides for rAAVs composed of engineered AAV5 VP1 capsid polypeptides having a SEQ ID NO: 2, wherein the Xaa1 to Xaa9 region is any one of SEQ ID NO: 17118-SEQ ID NO: 18117, as disclosed in TABLE 59. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 59
Sequences of the 581 to 589 Region in AAV5
VP1 Capsid Polypeptide that Drive Lung
Tissue Tropism

| SEQ ID | 581-589 |
|---|---|
| NO | Sequence |
| 17118 | AAKAIHYWM |
| 17119 | AAMPFYWFE |
| 17120 | AANKMNAER |
| 17121 | ACSTVMDFS |
| 17122 | ACSTVTDEN |
| 17123 | ADADMMWMA |
| 17124 | ADCSSSDWS |
| 17125 | AEKAIHYWM |
| 17126 | AFCCDHNAA |
| 17127 | AFEDYGSWE |
| 17128 | AFKAPRRGV |
| 17129 | AFKATHRGV |
| 17130 | AFKATRGGV |
| 17131 | AFKATRLGV |
| 17132 | AFKATRQGV |
| 17133 | AFKATRRAV |
| 17134 | AFKATRRGA |
| 17135 | AFKATRRGM |
| 17136 | AFKATRRSV |
| 17137 | AFKSTRRGV |
| 17138 | AFKTTRRGV |
| 17139 | AFMPKESKE |
| 17140 | AFNPQEMWA |
| 17141 | AFNQQEVWA |
| 17142 | AFQSTDWGW |
| 17143 | AFVHWPIHG |
| 17144 | AFVNWPLHG |
| 17145 | AFVNWTIHG |
| 17146 | AFVTWPIHG |
| 17147 | AGDFSGYYG |
| 17148 | AGETNNGGY |
| 17149 | AGSASNWAD |
| 17150 | AGVFRKITM |
| 17151 | AGYASHWAD |
| 17152 | AGYASNWGD |
| 17153 | AHCHFSNMQ |
| 17154 | AHMVENDTP |
| 17155 | AHWDFSNMQ |
| 17156 | AIHDQLEAM |
| 17157 | AKAFIEHTY |
| 17158 | AKEQGAVGR |
| 17159 | AKGAYNQGN |
| 17160 | AKGFLEHTY |
| 17161 | AKGSYNQGN |
| 17162 | AKGTYNHGN |
| 17163 | AKHFYQKFH |
| 17164 | AKISAAWDR |
| 17165 | AKNAMHNFF |
| 17166 | AKNYMHCYW |
| 17167 | AKRDHHYFA |
| 17168 | AKTDNHVLH |
| 17169 | ALTYCHWTD |
| 17170 | ALTYCNWAD |
| 17171 | AMKNLKRTG |
| 17172 | AMVCQAIVF |
| 17173 | AMVCQIIVF |
| 17174 | AMVCRVIVF |
| 17175 | ANSSNHSQE |
| 17176 | AQHNSESNI |
| 17177 | ARHVNFEMS |
| 17178 | ASKMSHKEA |
| 17179 | ASKRSGKQP |
| 17180 | ASMEHMKKA |
| 17181 | ASRIYCRHE |
| 17182 | ASRSLERQA |
| 17183 | ASYFMEITR |
| 17184 | ATEQWDFCR |
| 17185 | ATSVFCVFR |
| 17186 | ATTSNNLTR |
| 17187 | ATTSSNLTR |
| 17188 | ATTSYNLAR |
| 17189 | AVDPTFKPH |
| 17190 | AVEFLAHQY |
| 17191 | AVKCTEYSA |
| 17192 | AVNPAFKPH |
| 17193 | AVYPLFAPE |
| 17194 | AWKMMHCDI |
| 17195 | AWKMMHCHN |
| 17196 | AWKMMHFDN |
| 17197 | AWKMMHGDN |
| 17198 | AWKMMNCDN |
| 17199 | AWQMMHCDN |
| 17200 | AWRMMHCDN |
| 17201 | AWYASNWAD |
| 17202 | AYKNCHRHG |
| 17203 | AYRMFNNVQ |
| 17204 | CAKACDTSS |
| 17205 | CAKDCDASS |
| 17206 | CAKNCDTSS |
| 17207 | CARDCDTSS |
| 17208 | CDYVQREWD |
| 17209 | CDYVQRKWD |
| 17210 | CDYVQRRWD |
| 17211 | CERRALKMI |
| 17212 | CESIMHPDQ |
| 17213 | CFKWTIVNG |
| 17214 | CFRQDRIIF |
| 17215 | CIESFIENH |
| 17216 | CIWQHNVTQ |
| 17217 | CKAHFGFME |
| 17218 | CKDHFGFMV |
| 17219 | CKDHFGVME |
| 17220 | CKEAFDVNE |
| 17221 | CLRQDRIIS |
| 17222 | CPEAFDVNE |
| 17223 | CQDHFGFME |
| 17224 | CSKDCDTSS |
| 17225 | CSPPIVMDR |
| 17226 | CSYFMEITR |
| 17227 | CTAPIVMDR |
| 17228 | CTKDCDTSS |
| 17229 | CTPPIAMDR |
| 17230 | CTPPIFMDR |
| 17231 | CTPPIIMDR |
| 17232 | CTPPILMDR |
| 17233 | CTPPIVTDR |
| 17234 | CTPPTVMDR |
| 17235 | CTSFACKSF |
| 17236 | CVKDCDTSS |
| 17237 | DADNYMIYQ |
| 17238 | DADYYMIYR |
| 17239 | DARVSNIRQ |
| 17240 | DCRWYGNNS |
| 17241 | DCTNNELKR |
| 17242 | DCTNNKVKR |
| 17243 | DCTNTKLKR |
| 17244 | DEPCEELAF |
| 17245 | DFIQSDTEF |
| 17246 | DFVQSDTGF |
| 17247 | DGCSLYAFS |
| 17248 | DGDYYMIYQ |
| 17249 | DGTNNKLKR |
| 17250 | DHCGPIAWP |
| 17251 | DHPYQHIEG |
| 17252 | DICCNYDAQ |
| 17253 | DIGMRPSGW |
| 17254 | DKHFYEKFH |
| 17255 | DKNYMHCHW |
| 17256 | DKNYMHWYW |
| 17257 | DKNYMHYYW |
| 17258 | DKNYTHCYW |
| 17259 | DKRDNHYFA |
| 17260 | DKRDYHYFA |
| 17261 | DKRNHHYFA |
| 17262 | DKRYHHYFA |
| 17263 | DKSYMHCYW |
| 17264 | DKTYMHCYW |
| 17265 | DMSHTFVAA |
| 17266 | DNAFVHCNQ |
| 17267 | DNAIVHCDQ |
| 17268 | DNAIVHCNE |
| 17269 | DNAIVHCNL |
| 17270 | DNAMVHCNQ |
| 17271 | DNAVVHCNQ |
| 17272 | DNGIVHCNQ |
| 17273 | DRCMQAKHG |
| 17274 | DRCMQTKHA |
| 17275 | DRNYMHCYW |
| 17276 | DSAIVHCNQ |
| 17277 | DSKEEGLAF |
| 17278 | DSMAMSARA |
| 17279 | DSMGMTARA |
| 17280 | DSREEGLTF |
| 17281 | DSRQLHHLG |
| 17282 | DSRQLHHTG |
| 17283 | DSRQLHRIG |
| 17284 | DSRQLHYIG |
| 17285 | DSRRLHHIG |
| 17286 | DTAIVHCNQ |
| 17287 | DTNQLNDLS |
| 17288 | DTTPNESDR |
| 17289 | DVCTVFVTA |
| 17290 | DVDYYMIYQ |
| 17291 | DVVRVQKMA |
| 17292 | DWASNNWWP |
| 17293 | DWMTRHKVI |
| 17294 | DWMTRNKVI |
| 17295 | DWMTRYKLI |
| 17296 | DWMTRYKVL |
| 17297 | DWSANNWWP |
| 17298 | EAEYWSCGR |
| 17299 | EAEYWTCGR |
| 17300 | EAHYTNECR |
| 17301 | EARDSCWWG |
| 17302 | EARYCCWWG |
| 17303 | EARYSCWSG |
| 17304 | EARYTCWWG |
| 17305 | ECKQQGVRR |
| 17306 | EFCEMQSLC |
| 17307 | EFCHALVCQ |
| 17308 | EFEDYGGWE |
| 17309 | EFEMSVFSN |
| 17310 | EFFIFGAID |
| 17311 | EFFIIGAIG |
| 17312 | EFFIIGAIV |
| 17313 | EFFIIGAVD |
| 17314 | EFFIIGTID |
| 17315 | EFFIIVAID |
| 17316 | EFFIVGAID |
| 17317 | EFGHAMVCQ |
| 17318 | EFITSVQCS |
| 17319 | EFKKEFEAF |
| 17320 | EFKMSGFSN |
| 17321 | EFKMSVFSS |
| 17322 | EFKMSVFTN |
| 17323 | EFKMSVISN |
| 17324 | EFKMSVVSN |
| 17325 | EFKMTVFSN |
| 17326 | EFKQEFVAF |
| 17327 | EFKQEYEAF |
| 17328 | EFRMSVFSN |
| 17329 | EFTRKSGKF |
| 17330 | EFTRRSCKF |
| 17331 | EHGCTHMGN |
| 17332 | EHKQQGVRR |
| 17333 | EHQPFSMFS |
| 17334 | EHRQMHDAY |
| 17335 | EHRQMHDSF |
| 17336 | EHRQMHDSS |
| 17337 | EHSCNHMGN |
| 17338 | EHSYTHMGN |
| 17339 | EHTRQDVCD |
| 17340 | EKDHFGVMQ |
| 17341 | EKHHFDVMQ |
| 17342 | EKLMYDKDL |
| 17343 | EKLNHETGY |
| 17344 | EKLSHEAGY |
| 17345 | EKLTHEAGY |
| 17346 | EKMGYVHKG |
| 17347 | EMKDLKRTG |
| 17348 | EMKNLKRAG |
| 17349 | EMKNLKRNG |
| 17350 | EMKNLKRTC |
| 17351 | EMKNLKRTS |
| 17352 | EMKNLRRTG |
| 17353 | EMRPQKMSR |
| 17354 | ENAQFLVWP |
| 17355 | ENAQILVWA |
| 17356 | ENAQVLVWA |
| 17357 | ENAQYLVWA |
| 17358 | ENDSVSAIA |
| 17359 | ENEFIYCHP |
| 17360 | ENEWTYHQG |
| 17361 | ENGQFLVWA |
| 17362 | ENHRITGKS |
| 17363 | ENHSVSAIG |
| 17364 | ENSQASAFG |
| 17365 | ENSQFLVWA |
| 17366 | ENSQNSAFG |
| 17367 | ENSQTSPFG |
| 17368 | ENVMHSEFR |
| 17369 | ENVQFLVWA |
| 17370 | ENYRITGQS |
| 17371 | EPRPCNFWC |
| 17372 | EPRSCNFWG |
| 17373 | EPRSCNFWW |
| 17374 | EPRSCNYWC |
| 17375 | EQMGYVHKA |
| 17376 | ERAEFEMKQ |
| 17377 | ERNDFHWYM |
| 17378 | ERSCTHMGN |
| 17379 | ERSDFHWCM |
| 17380 | ERSEFEMKE |
| 17381 | ERSGFHWYM |
| 17382 | ESKMNHKEA |
| 17383 | ESKMSHKQA |
| 17384 | ESMFRDSDK |
| 17385 | ESMGICHNS |
| 17386 | ETIRDMSGF |
| 17387 | ETMAQSCYR |
| 17388 | ETRSCNFWC |
| 17389 | EVASQFFEH |
| 17390 | EVFKIDMIP |
| 17391 | EVGEMGILR |
| 17392 | EVGRIGWMS |
| 17393 | EVQKWFRKS |
| 17394 | EVRYSCWWG |
| 17395 | EVTDEHCCW |
| 17396 | EVTNEHCCL |
| 17397 | EVTNEHGCW |
| 17398 | EVVKIDMIS |
| 17399 | EVVKIDMTP |
| 17400 | EWKMMHCDN |
| 17401 | EWWRYMAYY |
| 17402 | EYICFNCYH |
| 17403 | EYKEQGVRR |
| 17404 | EYKKQGVRR |
| 17405 | EYKMSHKEA |
| 17406 | EYKPQGVRR |
| 17407 | EYKQEGVRR |
| 17408 | EYKQKGVRR |
| 17409 | EYKQPGVRR |
| 17410 | EYKQQCVRR |
| 17411 | EYKQQGLRR |
| 17412 | EYKQQSVRR |
| 17413 | EYQQQGVRR |
| 17414 | EYRQQGVRR |
| 17415 | EYTKQGVRR |
| 17416 | FARKKCDTW |
| 17417 | FCKLQKLKA |
| 17418 | FCKLRKLKG |
| 17419 | FCKLRKLRA |
| 17420 | FCKMRKLKA |
| 17421 | FCKSRKLKA |
| 17422 | FEAPFNLSF |
| 17423 | FEAPINLGF |
| 17424 | FEAPINMSF |
| 17425 | FEVPINLSF |
| 17426 | FFKLRKLKA |
| 17427 | FGAPINLSF |
| 17428 | FGKLRKLKA |
| 17429 | FGRYRLLKG |
| 17430 | FGRYRLWEG |
| 17431 | FGRYRLWKA |
| 17432 | FGRYRLWQV |
| 17433 | FGRYRLWRG |
| 17434 | FHKKFHSKF |
| 17435 | FHKQFHSEF |
| 17436 | FHKQFHSRF |
| 17437 | FHKQYHSKF |
| 17438 | FLKQFHSKF |
| 17439 | FNIDCFNAA |
| 17440 | FNKQFHSKF |
| 17441 | FNVDCFSAA |
| 17442 | FRKQFHSKF |
| 17443 | GDYVQRQWD |
| 17444 | GERWAHNML |
| 17445 | GERWPHHML |
| 17446 | GERWPHNMW |
| 17447 | GERWPNNML |
| 17448 | GERWTHNML |
| 17449 | GFDAEHGYQ |
| 17450 | GGNMRSSMI |
| 17451 | GHIHARDGA |
| 17452 | GIKQTLVRR |
| 17453 | GIRLKGDIM |
| 17454 | GIRLRGNIM |
| 17455 | GKRWPHNML |
| 17456 | GLRQDRIIF |
| 17457 | GNQPTHSPR |
| 17458 | GSETNNGGY |
| 17459 | GSIIESSAR |
| 17460 | GSIIKSSAR |
| 17461 | GSIIQGSAR |
| 17462 | GSISQSSAR |
| 17463 | GSIVQSSAR |
| 17464 | GSKMSHKEA |
| 17465 | GSMIQSSAR |
| 17466 | GSNIQSSAR |
| 17467 | GSQPTHSPR |
| 17468 | GTPPIVMDR |
| 17469 | GTRLKGNIM |
| 17470 | GVEFLAHRY |
| 17471 | GVKDNNRRL |
| 17472 | GWDKLMMLA |
| 17473 | GWHCFSFVV |
| 17474 | GWKMMHCDN |
| 17475 | GWNCFSFVV |
| 17476 | GYEPTHSPR |
| 17477 | GYKDCHRHG |
| 17478 | GYKPTHSPR |
| 17479 | GYQMFNNVQ |
| 17480 | GYQPTHSHR |
| 17481 | GYQPTNSPR |
| 17482 | GYRPTHSPR |
| 17483 | HATGVGKDR |
| 17484 | HAVTHGWHR |
| 17485 | HDAQFLPDE |
| 17486 | HGDHFDRNS |
| 17487 | HIEMRNSHM |
| 17488 | HIEMRNTHM |
| 17489 | HIEMRNVHM |
| 17490 | HIKCTHEDN |
| 17491 | HIQCTHEDD |
| 17492 | HIQCTHKDN |
| 17493 | HIQCTHQDN |
| 17494 | HMQCTHEDS |
| 17495 | HRIYIMKKM |
| 17496 | HRIYIMKQM |
| 17497 | HRNYIMKEM |
| 17498 | HSAGVGKDR |
| 17499 | HSEVKCVER |
| 17500 | HSNGVGKDR |
| 17501 | HSPTYKWKR |
| 17502 | HSTGIGKDR |
| 17503 | HSTGLGKDR |
| 17504 | HSTVVGKDR |
| 17505 | HTDGLFTDD |
| 17506 | HTKMTHGTA |
| 17507 | HTNCLFTDD |
| 17508 | HTRSKDFWN |
| 17509 | HVAANYDTF |
| 17510 | HVASNYDTS |
| 17511 | HVGTHGWHR |
| 17512 | HVITHGWHR |
| 17513 | HVVHLDDYW |
| 17514 | HVVTHGWQR |
| 17515 | HVVTLGWHR |
| 17516 | HVVTNGWHR |
| 17517 | IAIKQRDGA |
| 17518 | IANKQQLAR |
| 17519 | IAVKQPDGA |
| 17520 | IAVPCASFY |
| 17521 | IAVPSCWAR |
| 17522 | ICHDCMNYR |
| 17523 | ICVPSCWAR |
| 17524 | IEHDFNSAF |
| 17525 | IFHDCMNSR |
| 17526 | IFHDFMNYR |
| 17527 | IFSPQQLGA |
| 17528 | IGEKPVTDR |
| 17529 | IGGMFSRNA |
| 17530 | IGGMYSRHA |
| 17531 | IGRDIDTAF |
| 17532 | IGRDVDTSF |
| 17533 | IHEWIANVE |
| 17534 | IHEWLANVQ |
| 17535 | IHGLIPMDS |
| 17536 | IHKQFHSKF |
| 17537 | IHQWIANVQ |
| 17538 | IIANHSDMA |
| 17539 | IIANHSHMS |
| 17540 | IIANHSHMV |
| 17541 | IIKPTRAPA |
| 17542 | IIVNHSHMA |
| 17543 | IKHDFHSAF |
| 17544 | IKHDFNNAF |
| 17545 | IKHDFNSAC |
| 17546 | IKHDFNSDF |
| 17547 | IKHDFNSGF |
| 17548 | IKHDFNSPF |
| 17549 | IKHDFNSSF |
| 17550 | IKHDINSAF |
| 17551 | IKHDYNSAF |
| 17552 | IKHNFNSAF |
| 17553 | IKNAMHNFF |
| 17554 | IKNDFNSAF |
| 17555 | IKRDFNSAF |
| 17556 | IKYDFNSAF |
| 17557 | ILLYSCVKD |
| 17558 | IMLYSCVQD |
| 17559 | INATPNLRT |
| 17560 | INRENRRGC |
| 17561 | INVDCFNAA |
| 17562 | IQHDFNSAF |
| 17563 | IRADLDVDH |
| 17564 | IRHDFNSAF |
| 17565 | ISAIADLDR |
| 17566 | ISAIADMDQ |
| 17567 | ISAPPNLRT |
| 17568 | ISAPSCWAR |
| 17569 | ISDPSCWAR |
| 17570 | ISEDIKDNG |
| 17571 | ISEDIKENA |
| 17572 | ISEDIKENS |
| 17573 | ISGPSCWAR |
| 17574 | ISIPSCWAR |
| 17575 | ISLPSCWAR |
| 17576 | ISNKQQLAR |
| 17577 | ISVHSCWAR |
| 17578 | ISVPGCWAR |
| 17579 | ISVPICWAR |
| 17580 | ISVPRCWAR |
| 17581 | ISVPSCWER |
| 17582 | ISVPSCWGR |
| 17583 | ISVPSCWSR |
| 17584 | ISVPTCWAR |
| 17585 | ISVSSCWAR |
| 17586 | ISVTSCWAR |
| 17587 | ITNKEQLAR |
| 17588 | ITNKQELAR |
| 17589 | ITNKQLLAR |
| 17590 | ITNKQPLAR |
| 17591 | ITNKQQLER |
| 17592 | ITNKQQLGR |
| 17593 | ITNKQQLPR |
| 17594 | ITNKQQLSR |
| 17595 | ITNKQQQAR |
| 17596 | ITNKRQLAR |
| 17597 | ITSKQQLAR |
| 17598 | ITTKQQLSR |
| 17599 | ITTQFMSKH |
| 17600 | IVAISCYDN |
| 17601 | IVGISCYDD |
| 17602 | IVGNSCYDN |
| 17603 | IVGTSCYDN |
| 17604 | IVKDNNRRL |
| 17605 | IWEKPATDR |
| 17606 | IWEKPVADR |
| 17607 | IWEKPVTAR |
| 17608 | IWEKSVTDR |
| 17609 | IWEKTVTDR |
| 17610 | IYSPNRQDR |
| 17611 | IYVPSCWAR |
| 17612 | KAEYWNCGR |
| 17613 | KCEMISLSR |
| 17614 | KEFCCCCKA |
| 17615 | KFGSFWPTG |
| 17616 | KGDHFDRNS |
| 17617 | KHFCCCCKA |
| 17618 | KHVMHSEFR |
| 17619 | KHWRHTMFL |
| 17620 | KKDHFDVMQ |
| 17621 | KKEPCNSGH |
| 17622 | KLREVGRLC |
| 17623 | KMEWFPHNS |
| 17624 | KMKNLKRTG |
| 17625 | KMRQQKMSR |
| 17626 | KNAPTNQPC |
| 17627 | KNEWIYHQG |
| 17628 | KNEWNYHQG |
| 17629 | KNEWTYHQA |
| 17630 | KNEWTYHQC |
| 17631 | KNVKHSEFR |
| 17632 | KNVMHSAFR |
| 17633 | KNVMHSQFR |
| 17634 | KNVMHSVFR |
| 17635 | KPLIMWPDF |
| 17636 | KPLLMLPDF |
| 17637 | KPMLMWPDF |
| 17638 | KPRSCNFWC |
| 17639 | KPVLMWPDF |
| 17640 | KQAPCNSGH |
| 17641 | KQFCCCCRA |
| 17642 | KQFCCCRKA |
| 17643 | KQFCCCWKA |
| 17644 | KQFCFCCKA |
| 17645 | KQFCWCCKA |
| 17646 | KRFCCCCKA |
| 17647 | KSESAASDN |
| 17648 | KSEVKCVER |
| 17649 | KSKMSHKEA |
| 17650 | KSRIMAPLW |
| 17651 | KTDYCYQCL |
| 17652 | KTIRDMSGF |
| 17653 | KVGEMGIIR |
| 17654 | KVGEMGLLR |
| 17655 | KVGEMSILR |
| 17656 | KVGNEKSCI |
| 17657 | KVHTFFANE |
| 17658 | KVQPVRRFT |
| 17659 | KWEKPVTDR |
| 17660 | KYKQQGVRR |
| 17661 | LAAVSVFRR |
| 17662 | LACACQDAA |
| 17663 | LAETEKLWN |
| 17664 | LASANHGGN |
| 17665 | LCMHTMWSR |
| 17666 | LFDDVMVGR |
| 17667 | LFGESMVGT |
| 17668 | LGKVINERQ |
| 17669 | LGNFIDMPA |
| 17670 | LHYAFDQCM |
| 17671 | LKIPFNMTF |
| 17672 | LKKERGRAC |
| 17673 | LKKKRGRSC |
| 17674 | LQKKRGRAC |
| 17675 | LQSFRSMMD |
| 17676 | LQSPLLWER |
| 17677 | LRAPYNFWW |
| 17678 | LRIYIMKEM |
| 17679 | LRKKRGRAC |
| 17680 | LRTPFNFWW |
| 17681 | LRTPYDFWW |
| 17682 | LRTSYNFWW |
| 17683 | LRTTYNFWW |
| 17684 | LRYSFDQCM |
| 17685 | LSTGVGKDR |
| 17686 | LSYVQGDWR |
| 17687 | LTAVSGFRR |
| 17688 | LTFVQGDWR |
| 17689 | LTPVSVFRR |
| 17690 | LTRCNEKGN |
| 17691 | LTTGIWKNW |
| 17692 | LTYAQGDWR |
| 17693 | LTYVEGDWR |
| 17694 | LTYVKGDWR |
| 17695 | LTYVQGNWR |
| 17696 | LTYVQRDWR |
| 17697 | LVEFYSENL |
| 17698 | LVHELNQIK |
| 17699 | LVHNSPFDF |
| 17700 | LVKDEDNNC |
| 17701 | LVKMDWKEA |
| 17702 | LVNELNQIK |
| 17703 | LVRVINERQ |
| 17704 | LYCRMHNWA |
| 17705 | LYCRMNDWA |
| 17706 | MASIFQYPR |
| 17707 | MCDDLMVGR |
| 17708 | MCKSYERGA |
| 17709 | MCMHKMWSR |
| 17710 | MCYGFWCEI |
| 17711 | MFDDLMVAR |
| 17712 | MFDDLMVCR |
| 17713 | MFDDLMVGH |
| 17714 | MFDDLMVSR |
| 17715 | MFDDVMVGR |
| 17716 | MFDEVMVGR |
| 17717 | MFDNLMVGR |
| 17718 | MFDYLMVGR |
| 17719 | MFGDLMVGR |
| 17720 | MFHDCMNYR |
| 17721 | MFNDLMVGR |
| 17722 | MGIRDWKRV |
| 17723 | MGIREWRRV |
| 17724 | MGIWKSQRH |
| 17725 | MGTREWKRV |
| 17726 | MHNDKLMDE |
| 17727 | MHNDKWMDA |
| 17728 | MHSPNRQDR |
| 17729 | MHYSFDQCM |
| 17730 | MIHWDYHMK |
| 17731 | MIHWDYNME |
| 17732 | MIHWDYQME |
| 17733 | MILWDYHME |
| 17734 | MINWDYHME |
| 17735 | MIRSETQWA |
| 17736 | MIWSNRKFM |
| 17737 | MIWYNRKCM |
| 17738 | MIWYNRKVM |
| 17739 | MIWYNRQFM |
| 17740 | MIYWDYHME |
| 17741 | MKDEHDDMP |
| 17742 | MKDEQDDTP |
| 17743 | MKETMDCSY |
| 17744 | MKKAHDRSA |
| 17745 | MKKKRGRAC |
| 17746 | MKLDYDVGC |
| 17747 | MKNEKNDNP |
| 17748 | MKWDYDVGG |
| 17749 | MMKPWACLE |
| 17750 | MMKPWVFLE |
| 17751 | MMKQWVCLE |
| 17752 | MMMMRATMD |
| 17753 | MMMMRVAMD |
| 17754 | MNTALFVNA |
| 17755 | MQIKVSWNR |
| 17756 | MQKAHDRAA |
| 17757 | MQKEHDRSA |
| 17758 | MQKPHDRSA |
| 17759 | MQKTHDRSA |
| 17760 | MQKWTEIGC |
| 17761 | MQLKGSWNR |
| 17762 | MQNEKNDHP |
| 17763 | MQNEKNDTP |
| 17764 | MQNEKNENP |
| 17765 | MQTKGSWNR |
| 17766 | MRDEQDDMP |
| 17767 | MRNDKWMDE |
| 17768 | MRNEKNDNP |
| 17769 | MRTPYNFWW |
| 17770 | MSRTCADWT |
| 17771 | MTAVSVFRR |
| 17772 | MTKQNKIHR |
| 17773 | MTSIFQHPR |
| 17774 | MTYVQGDWR |
| 17775 | MVDGLMKEY |
| 17776 | MVIREWKRV |
| 17777 | MVKCTEYSA |
| 17778 | MVKVINERQ |
| 17779 | MVWYNRKFM |
| 17780 | MWKCKSTYA |
| 17781 | MYNPNRQDR |
| 17782 | MYSANRQDR |
| 17783 | MYSPNHQDR |
| 17784 | MYSPNREDR |
| 17785 | MYSPNRHDR |
| 17786 | MYSPNRKDR |
| 17787 | MYSPNRQAR |
| 17788 | MYSQNRQDR |
| 17789 | MYSTNRQDR |
| 17790 | MYTPNRQDR |
| 17791 | NASNCFWCF |
| 17792 | NASTCFWCC |
| 17793 | NCRCYGNNS |
| 17794 | NESPYHMCL |
| 17795 | NFKTYRFCE |
| 17796 | NFVQYVHNY |
| 17797 | NGSNCFWCC |
| 17798 | NHAPAKNKA |
| 17799 | NICCNYDAK |
| 17800 | NICCNYDAP |
| 17801 | NKNAMHNFF |
| 17802 | NKNYMHCYW |
| 17803 | NLEMLAMFP |
| 17804 | NLEMLAMFR |
| 17805 | NMRSTEMGV |
| 17806 | NNAPIWLHG |
| 17807 | NNGVFHWKD |
| 17808 | NNPYQHIEG |
| 17809 | NNSVFHWKD |
| 17810 | NNVKIQYMG |
| 17811 | NNWAFHWKD |
| 17812 | NNWGFHWKD |
| 17813 | NNWVCHWKD |
| 17814 | NNWVFHWKA |
| 17815 | NNWVFHWKH |
| 17816 | NNWVFPWKD |
| 17817 | NNWVIHWKD |
| 17818 | NPRSKDFWN |
| 17819 | NQCGLKMYP |
| 17820 | NSAIKEEMA |
| 17821 | NSAIRQEMA |
| 17822 | NSDMKETGD |
| 17823 | NSRQLHHIG |
| 17824 | NSRSKDFWN |
| 17825 | NSSNCFWCC |
| 17826 | NTEPMWSCS |
| 17827 | NTRPRRLHM |
| 17828 | NTRRMRLHM |
| 17829 | NTRSKDFWY |
| 17830 | NTSNCFWCC |
| 17831 | NVMPLIEYR |
| 17832 | NVMTLIQYR |
| 17833 | PAEINKMHC |
| 17834 | PQAKIDMEF |
| 17835 | PQSPLLWER |
| 17836 | PQVKVDMEF |
| 17837 | PRRLLHFSQ |
| 17838 | PRYMGTKDA |
| 17839 | PSRLEHYGL |
| 17840 | PTKMTHGTS |
| 17841 | PTSFAETDY |
| 17842 | PVHTFFANE |
| 17843 | QAHTFFANE |
| 17844 | QAISDMPFP |
| 17845 | QFFWSLHAV |
| 17846 | QFGTFWPTG |
| 17847 | QGEPLMRYS |
| 17848 | QHQAFSMFS |
| 17849 | QHQPFTMFS |
| 17850 | QHSCTHMGN |
| 17851 | QICPSFDAD |
| 17852 | QINPSFDAD |
| 17853 | QIYPSFEAD |
| 17854 | QIYTSFDAD |
| 17855 | QKATTDMYW |
| 17856 | QKSPLLWER |
| 17857 | QKTPTDMYW |
| 17858 | QMKNLKRTG |
| 17859 | QMVYAQKNM |
| 17860 | QNAWIECQL |
| 17861 | QNTWIQCQL |
| 17862 | QNVMHSEFR |
| 17863 | QPRSCNFWC |
| 17864 | QQIPLLWER |
| 17865 | QQQPFSMFS |
| 17866 | QQSPPLWER |
| 17867 | QQSPQLWDR |
| 17868 | QQSPQLWER |
| 17869 | QQSTLLWER |
| 17870 | QRHDLEVDL |
| 17871 | QSIRDMSGF |
| 17872 | QSQVKCVER |
| 17873 | QSRIMAPLW |
| 17874 | QSSFAETDY |
| 17875 | QTIRDMGGF |
| 17876 | QTIRDMNGF |
| 17877 | QTTFAETDY |
| 17878 | QTWAAHCTR |
| 17879 | QTWSAHCSR |
| 17880 | QTWTAHCSR |
| 17881 | QYVCFNCYH |
| 17882 | RACIPKFKE |
| 17883 | RACVPKFKQ |
| 17884 | RAEILQLQQ |
| 17885 | RARIPKFKQ |
| 17886 | RASIFQHPR |
| 17887 | RCDEWAEDQ |
| 17888 | RCDEWAKDK |
| 17889 | RCDEWAKDR |
| 17890 | RCDEWAKNQ |
| 17891 | RCDEWARDQ |
| 17892 | RCDEWVKDQ |
| 17893 | RCVPWMNQW |
| 17894 | RDAQFLPDQ |
| 17895 | RFDDLMVGR |
| 17896 | RGDEWAKDQ |
| 17897 | RGEILELQQ |
| 17898 | RGEILQLQR |
| 17899 | RHQPCECYE |
| 17900 | RHQPFSMFS |
| 17901 | RHWRHAMFL |
| 17902 | RHWRLTMFL |
| 17903 | RIMVHEWGD |
| 17904 | RLDHAETRA |
| 17905 | RMLAYKQRH |
| 17906 | RVDHAESRA |
| 17907 | RVVDLVDVY |
| 17908 | RWEKPVTDR |
| 17909 | SAEAAYENP |
| 17910 | SDCSYSDWS |
| 17911 | SEVHLEHNY |
| 17912 | SFCCDHNAG |
| 17913 | SFCCDHNAS |
| 17914 | SFCCDHNGA |
| 17915 | SFDERGVWC |
| 17916 | SFGSCNTCG |
| 17917 | SFHDEEGLF |
| 17918 | SFKATRRGV |
| 17919 | SFKLTIVNG |
| 17920 | SFKWPIVNG |
| 17921 | SFKWTILNG |
| 17922 | SFKWTIVDG |
| 17923 | SFKWTIVNA |
| 17924 | SFKWTIVND |
| 17925 | SFKWTIVNV |
| 17926 | SFKWTIVTG |
| 17927 | SGKDNNRRL |
| 17928 | SHIHKDSCW |
| 17929 | SIMKYDEMG |
| 17930 | SIMKYDQMV |
| 17931 | SIMLNECNT |
| 17932 | SIRLKGNIM |
| 17933 | SKISATWDR |
| 17934 | SKLHKQYCC |
| 17935 | SKYDTDSAC |
| 17936 | SLEMRPRQI |
| 17937 | SLKDNNRRL |
| 17938 | SLKMRPRKI |
| 17939 | SLKMRPRQS |
| 17940 | SLKMRPRQT |
| 17941 | SLQMRPRQI |
| 17942 | SLTASNWRD |
| 17943 | SMVCQVIVF |
| 17944 | SNDKNIHNG |
| 17945 | SNWVFHWKD |
| 17946 | SPREKNDYW |
| 17947 | SPREKNRYW |
| 17948 | SRIHKDICW |
| 17949 | SRINKDSCW |
| 17950 | SRLHKDSCW |
| 17951 | SRTMKDWWG |
| 17952 | SSAIKQEMA |
| 17953 | SSEQTGPHM |
| 17954 | SSHFMEITR |
| 17955 | SSIIQSSAR |
| 17956 | SSKQAGPHM |
| 17957 | SSRPRMKQP |
| 17958 | SSTASNWRD |
| 17959 | SSYFMAITR |
| 17960 | SSYFMEIAR |
| 17961 | SSYFMEVTR |
| 17962 | SSYFMKITR |
| 17963 | SSYFTEITR |
| 17964 | SSYIMEITR |
| 17965 | SSYVMEITR |
| 17966 | SSYYMEITR |
| 17967 | STDDKFWLG |
| 17968 | STDNRFWLG |
| 17969 | STDTKFWLG |
| 17970 | STEAAHENP |
| 17971 | STEASYENP |
| 17972 | STESAYENP |
| 17973 | STQQVFWNL |
| 17974 | STRPMRLHM |
| 17975 | STSILDKLI |
| 17976 | STSSLDKLV |
| 17977 | STTAYNLTR |
| 17978 | SVHAKMEGC |
| 17979 | SVHSKMQGC |
| 17980 | SVHSTMEGC |
| 17981 | SVKDINRRL |
| 17982 | SVKDNNGRL |
| 17983 | SVKDNNQRL |
| 17984 | SVKDNNRIL |
| 17985 | SVKDNNRKL |
| 17986 | SVKDNNRTL |
| 17987 | SVKDTNRRL |
| 17988 | SVKNNNRRL |
| 17989 | SVMQSIDSS |
| 17990 | SVNSKMEGC |
| 17991 | SVRDNNRRL |
| 17992 | SWAASNWRD |
| 17993 | SWKMMHCDN |
| 17994 | SWTSSNWRD |
| 17995 | SWTTSNWRD |
| 17996 | SWTVSNWRD |
| 17997 | SYQMFNNVQ |
| 17998 | TAQYLNREI |
| 17999 | TAREDYQHA |
| 18000 | TARLLEKYN |
| 18001 | TCMGFTSFP |
| 18002 | TFFVTASWS |
| 18003 | TFHQCERSV |
| 18004 | TFKNYRFCE |
| 18005 | TFKTYRYCE |
| 18006 | TFPNPVHRG |
| 18007 | TFRTYRFCE |
| 18008 | TFSNPVHRV |
| 18009 | TFVNWPIHG |
| 18010 | TKIFTENDQ |
| 18011 | TKNAMHHFF |
| 18012 | TKNDMHNFF |
| 18013 | TKNGMHNFF |
| 18014 | TKNPMHNFF |
| 18015 | TKTAMHNFF |
| 18016 | TLTTEMIWA |
| 18017 | TNWVFHWKD |
| 18018 | TPEAEHHKI |
| 18019 | TRGMRDFCY |
| 18020 | TRMVLEWTY |
| 18021 | TSEVFTVNC |
| 18022 | TSQYVNREI |
| 18023 | TSVPSCWAR |
| 18024 | TTEQWDVCR |
| 18025 | TTEVEHHKI |
| 18026 | TVKCTGYSA |
| 18027 | TVKDENAAC |
| 18028 | TVKEENAAF |
| 18029 | TVKEENATC |
| 18030 | TVKEENSAC |
| 18031 | TVKEENTAC |
| 18032 | TVRCTEYSA |
| 18033 | TWKMMHCDN |
| 18034 | VARPWAMNS |
| 18035 | VATQSCKWM |
| 18036 | VAVTITDFD |
| 18037 | VAVTVRDFD |
| 18038 | VCKWMGRQQ |
| 18039 | VCSTVMDFN |
| 18040 | VEADNNDAD |
| 18041 | VFNQQEMWA |
| 18042 | VGKWMGKQQ |
| 18043 | VGVTVTDFD |
| 18044 | VHALIPMDS |
| 18045 | VHGLIPMDT |
| 18046 | VHRQMHDSY |
| 18047 | VICATQDAA |
| 18048 | VICATQDPS |
| 18049 | VICPTQDPA |
| 18050 | VICTTQDPA |
| 18051 | VIHDQLETM |
| 18052 | VINMNRRWS |
| 18053 | VKKKRGRAC |
| 18054 | VKNYMHCYW |
| 18055 | VKSFRSMMD |
| 18056 | VMRWPWDDT |
| 18057 | VPRSCNFWC |
| 18058 | VRTPYNFWW |
| 18059 | VSETNNGGY |
| 18060 | VSINTNQPG |
| 18061 | VSKMSHKEA |
| 18062 | VSKPIEVGY |
| 18063 | VSMSSIYDK |
| 18064 | VSMSSNSDK |
| 18065 | VSNQCCKAM |
| 18066 | VSRALERQA |
| 18067 | VSRSLERKA |
| 18068 | VSRSLERQG |
| 18069 | VSRSLERQT |
| 18070 | VSRSLKRQA |
| 18071 | VSRSLQRQA |
| 18072 | VSRSQERQA |
| 18073 | VSVPSCWAR |
| 18074 | VSVTVTDFD |
| 18075 | VTEHQQRGV |
| 18076 | VTKCNEKGN |
| 18077 | VTNKQQLAR |
| 18078 | VTNQCCKTM |
| 18079 | VTSQCCKAM |
| 18080 | VTTGIWKTW |
| 18081 | VVGGDKAAY |
| 18082 | VVKVINERQ |
| 18083 | VVSQTYRNR |
| 18084 | VWKQYILVI |
| 18085 | VWMQHMCLD |
| 18086 | VWQQYILII |
| 18087 | WCRQTMMDL |
| 18088 | WDAMDDKKA |
| 18089 | WETDMDMDA |
| 18090 | WFRRHDYFY |
| 18091 | WFRRHSYFY |
| 18092 | WGDKKATDR |
| 18093 | WGDKMAADA |
| 18094 | WGDKPATDR |
| 18095 | WGDKRATDR |
| 18096 | WKNDMDMDA |
| 18097 | WKTEMDMDA |
| 18098 | WNCVMPDNA |
| 18099 | WNTDMDMDA |
| 18100 | WVDKQATDR |
| 18101 | YCRCYGNNS |
| 18102 | YIMVHEWGD |
| 18103 | YMILQHDAL |
| 18104 | YMTLEHDAL |
| 18105 | YQSGGILRM |
| 18106 | YQSGGIRRM |
| 18107 | YQTGGIHRM |
| 18108 | YSHTYKWKR |
| 18109 | YSLTYKWKR |
| 18110 | YSPTYMWKR |
| 18111 | YSQLFTFFE |
| 18112 | YSSTYKWKR |
| 18113 | YSTTYGGRH |
| 18114 | YVTEVNSMI |
| 18115 | YWVDFVMPF |
| 18116 | YWVNFVVPF |
| 18117 | YWVTFVMPF |

### Example 38

AAV5 Variants with Sciatic Nerve Tissue Tropism as Identified by Machine Learning

This example describes preferred properties of engineered AAV5 variants that display sciatic nerve tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display sciatic nerve tissue tropism.

Favored biophysical properties and favored amino acid residues at each position in the 581 to 589 region of an AAV5 VP capsid polypeptide, which are associated with a higher probability of sciatic nerve tissue tropism are described below. Any of the below machine learning-derived positional amino acid preferences described in TABLE 60 can be present alone or in combination with each other or in combination with any of the amino acid preferences observed and described in EXAMPLE 8, TABLE 15. Listed below in TABLE 60 are a summary of positional features shared between the top important features for sciatic nerve tropism extracted from the ML models.

TABLE 60
Machine Learning-Derived Sciatic Nerve Tissue Tropism Rules

High average flexibility at Xaa1
Xaa1 is selected from G or R
Low solubility at Xaa1
Xaa1 is selected from R or Q
Low mutability at Xaa1
Xaa1 is selected from C, L, F, Y, R, K, P, or H
High volume at Xaa1
Xaa1 is selected from Y or F
High surface accessibility at Xaa2
Xaa2 is selected from E, R, or K
Medium mutability at Xaa3
Xaa3 is selected from H or R
Medium average flexibility at Xaa3
Xaa3 is selected from V or Y
High mutability at Xaa4
Xaa4 is selected from N
High average flexibility at Xaa4
Xaa4 is selected from I, N, G, or R
Low solubility at Xaa4
Xaa4 is selected from N
Low mutability at Xaa6
Xaa6 is selected from C, L, F, or Y
High volume at Xaa6
Xaa6 is selected from K, M, I, or L
Low mutability at Xaa7
Xaa7 is selected from L, F, or Y
Medium mol mass at Xaa7
Xaa7 is selected from D, I, L, or N
High surface accessibility at Xaa8
Xaa8 is selected from S, Y, T, D, P, H, or N
Low mutability at Xaa9
Xaa9 is selected from C, H, or R
Medium solubility at Xaa9
Xaa9 is selected from Q, T, or C
Low surface accessibility at Xaa9
Xaa9 is selected from C

TABLE 61 below provides sequences of the 581 to 589 region for variant AAV VP1 capsid polypeptides that exhibited sciatic nerve tissue tropism and comport to one or more of the rules provided in TABLE 60. The present disclosure, thus, provides for rAAVs composed of engineered AAV5 VP1 capsid polypeptides having a SEQ ID NO: 2, wherein the Xaa1 to Xaa9 region is any one of SEQ ID NO: 26991-SEQ ID NO: 27990, as disclosed in TABLE 61. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 61
Sequences of the 581 to 589 Region in AAV5
VP1 Capsid Polypeptide that Drive Sciatic
Nerve Tissue Tropism

| SEQ | 581-589 |
|---|---|
| ID NO | Sequence |
| 26991 | AAYLYNVPM |
| 26992 | AIRIAMCDW |
| 26993 | AIRTAMCDW |
| 26994 | AIRTTMCHW |
| 26995 | ANGCAIEYH |
| 26996 | ANGCTIENH |
| 26997 | ANGGAIENH |
| 26998 | ANGGAIENN |
| 26999 | ARYRMRYHG |
| 27000 | ASGCAIENH |
| 27001 | ATFGWMNWF |
| 27002 | ATSRQAVWA |
| 27003 | ATYLYNIPM |
| 27004 | AVLLWCEVQ |
| 27005 | AYPRNAKYI |
| 27006 | AYSRNAKYT |
| 27007 | CADWPCMSL |
| 27008 | CAGWACMSL |
| 27009 | CAKFNNAIH |
| 27010 | CAKFTNAIY |
| 27011 | CAKFTNTIH |
| 27012 | CAKFTTAIH |
| 27013 | CANWACMSL |
| 27014 | CCCEALMPK |
| 27015 | CCCEALMSQ |
| 27016 | CCCEAMMPQ |
| 27017 | CCCEAVMPQ |
| 27018 | CCCEVLMPQ |
| 27019 | CCCPDATNN |
| 27020 | CCCSDATNN |
| 27021 | CCGQVEFGW |
| 27022 | CCSGALMPQ |
| 27023 | CDGQVEFGW |
| 27024 | CEDPLCQSS |
| 27025 | CEDWACMSL |
| 27026 | CEHPLCQSS |
| 27027 | CEIPLCQSS |
| 27028 | CENPLCKSS |
| 27029 | CENPLCPSS |
| 27030 | CENPLCQSN |
| 27031 | CENPLGQSS |
| 27032 | CENPLROSS |
| 27033 | CENPLWQSS |
| 27034 | CENPQCQSS |
| 27035 | CENSLCQSS |
| 27036 | CETPLCQSS |
| 27037 | CEVNEEYDW |
| 27038 | CFAEPITDC |
| 27039 | CFGAEGYAG |
| 27040 | CFNEPITDC |
| 27041 | CFTAPITDC |
| 27042 | CFTEPIADC |
| 27043 | CFTEPINDC |
| 27044 | CFTEPITDR |
| 27045 | CFTEPITNC |
| 27046 | CFTESITDC |
| 27047 | CFTETITDC |
| 27048 | CGGKVEFGW |
| 27049 | CGGQAEFGW |
| 27050 | CGGQFEFGW |
| 27051 | CGGQIEFGW |
| 27052 | CGGQIEVGW |
| 27053 | CGGQVEAGW |
| 27054 | CGGQVECGW |
| 27055 | CGGQVEFDW |
| 27056 | CGGQVEFSL |
| 27057 | CGGQVEFSW |
| 27058 | CGGQVEFVW |
| 27059 | CGGQVEIGW |
| 27060 | CGGQVKFGW |
| 27061 | CGGQVQFGW |
| 27062 | CGKFTNAIH |
| 27063 | CGVQVEFGW |
| 27064 | CHPQHHYSH |
| 27065 | CHPQHRHSH |
| 27066 | CHPQLHHSH |
| 27067 | CKDTRKMRE |
| 27068 | CKEHRKMRE |
| 27069 | CKEPGKMRE |
| 27070 | CKEPRKMRA |
| 27071 | CKEPRKMRK |
| 27072 | CKEPRQMRE |
| 27073 | CKEPRRMRE |
| 27074 | CKETRKMRE |
| 27075 | CKKPRKMRE |
| 27076 | CKVNEEYDW |
| 27077 | CQIRFDRHH |
| 27078 | CONPLCQSS |
| 27079 | CQVNAEYDW |
| 27080 | CQVNEAYDW |
| 27081 | CQVNEEDDW |
| 27082 | CQVNEEFDW |
| 27083 | CQVNEEYNW |
| 27084 | CRCKDAWNM |
| 27085 | CRCNAAWNM |
| 27086 | CRCNDAGNM |
| 27087 | CRCNDAWHR |
| 27088 | CRCNDAWNK |
| 27089 | CRCNDAWNR |
| 27090 | CRCNDAWNV |
| 27091 | CRCNDAWTM |
| 27092 | CRCNDEWNM |
| 27093 | CRCNEAWNM |
| 27094 | CRCNGAWNM |
| 27095 | CRCSDAWNM |
| 27096 | CRFNDAWNM |
| 27097 | CRGQVEFGW |
| 27098 | CRVNEEYDW |
| 27099 | CSSCNHRVF |
| 27100 | CVFGWNNYQ |
| 27101 | CVGQVEFDW |
| 27102 | CVGQVEFGW |
| 27103 | CVKFTNAIH |
| 27104 | CVWSVGTEW |
| 27105 | CWAADSKPC |
| 27106 | CYPQHHHSH |
| 27107 | DDLDAWQTS |
| 27108 | DDLDLWQTS |
| 27109 | DDLDVGQTS |
| 27110 | DDLDVWETS |
| 27111 | DDLDVWKTS |
| 27112 | DDLDVWRTS |
| 27113 | DDMDVWQTS |
| 27114 | DDQDVWQTS |
| 27115 | DDRDVWQTS |
| 27116 | DFEMWDWVG |
| 27117 | DFEVWDWFG |
| 27118 | DFNHIGWWQ |
| 27119 | DFSHIAWWQ |
| 27120 | DFTHIAWWQ |
| 27121 | DFYFSGFLI |
| 27122 | DFYISGFLI |
| 27123 | DFYLSGFLI |
| 27124 | DPADKPSNS |
| 27125 | DPQDKPSNS |
| 27126 | DRIMISIQP |
| 27127 | DRVMISIQQ |
| 27128 | DRVMVSIQP |
| 27129 | DVMMPALKV |
| 27130 | DVMMRGLKV |
| 27131 | DVMMSGLKV |
| 27132 | DWRMVCVHC |
| 27133 | EACLYNIPM |
| 27134 | EAYFYNIPM |
| 27135 | EAYLCNIPM |
| 27136 | EAYLYHIPM |
| 27137 | EAYLYMIPM |
| 27138 | EAYLYNFPM |
| 27139 | EAYLYNIRM |
| 27140 | EAYLYNISM |
| 27141 | EAYLYNVPM |
| 27142 | EAYLYSIPM |
| 27143 | EAYMYNIPM |
| 27144 | EEYLYNIPM |
| 27145 | EFKRMTCNR |
| 27146 | EGSRKWFAC |
| 27147 | EGYLYNIPM |
| 27148 | EIGIMRHGH |
| 27149 | EIILDDEQR |
| 27150 | EIILDDQKR |
| 27151 | EIIVDDQQR |
| 27152 | EIIVDEQQR |
| 27153 | EIMLDDQQR |
| 27154 | EIVLDDQQR |
| 27155 | EKMIDTCLQ |
| 27156 | EKMRHTCLQ |
| 27157 | EKMTHPCLQ |
| 27158 | EKMTHTCLE |
| 27159 | EKMTHTCLK |
| 27160 | EKMTHTCLR |
| 27161 | EKMTHTCVQ |
| 27162 | EKMTHTCVR |
| 27163 | EKMTHTWLQ |
| 27164 | EPYLYNIPM |
| 27165 | EQMTHTCLP |
| 27166 | EQMTHTCLQ |
| 27167 | ERMTHTCLR |
| 27168 | ESHVRPLNM |
| 27169 | ETMKHKKRE |
| 27170 | ETMQYKKRE |
| 27171 | EWTRRMAAP |
| 27172 | EWTRRMEAS |
| 27173 | EWTRRMEAT |
| 27174 | EWTRRMEDP |
| 27175 | FAAANQNHM |
| 27176 | FAPIVKRHH |
| 27177 | FDLSFCYHQ |
| 27178 | FEKNSSQMR |
| 27179 | FGGQVELGW |
| 27180 | FHPYFPTYK |
| 27181 | FHVRFDRHH |
| 27182 | FIAANQNHM |
| 27183 | FKEPRKMRE |
| 27184 | FKIRFDRHH |
| 27185 | FKMDCGSCG |
| 27186 | FKMDCHGCG |
| 27187 | FKMDCHSCC |
| 27188 | FKMDCHSFG |
| 27189 | FKMDCHSGG |
| 27190 | FKMDCHTCG |
| 27191 | FKMDGHSCG |
| 27192 | FKMDWHSCG |
| 27193 | FKMGCHSCG |
| 27194 | FKMVCHSCG |
| 27195 | FKRDCHSCG |
| 27196 | FKRICLEAG |
| 27197 | FKRICWEAA |
| 27198 | FKRICWEGG |
| 27199 | FKRICWEPG |
| 27200 | FKRICWEVG |
| 27201 | FKRICWGAG |
| 27202 | FKRICWQAG |
| 27203 | FKRMCWEAG |
| 27204 | FKRTCWEAG |
| 27205 | FKRVCWEAG |
| 27206 | FKVDCHSCG |
| 27207 | FKVNRDDSW |
| 27208 | FKVRFDRHH |
| 27209 | FLIRFDRHH |
| 27210 | FNAGPLAYN |
| 27211 | FNLPFCYHQ |
| 27212 | FNLSFCYHL |
| 27213 | FNLSICYHQ |
| 27214 | FPGVMEAEI |
| 27215 | FPGVMQGEI |
| 27216 | FPHIVKRHH |
| 27217 | FPIRFDRHH |
| 27218 | FPIRFDRHL |
| 27219 | FPIRFDRHR |
| 27220 | FPIRFDRPH |
| 27221 | FPPIVKQHH |
| 27222 | FPPIVKRHY |
| 27223 | FPPTVKRHH |
| 27224 | FQIGFDRHH |
| 27225 | FQIKFDRHH |
| 27226 | FQILFDRHH |
| 27227 | FQIPFDRHH |
| 27228 | FQIQFDRHH |
| 27229 | FQIQIDRHH |
| 27230 | FQIRFARHH |
| 27231 | FQIRFDGHH |
| 27232 | FQIRFDRDH |
| 27233 | FQIRFDRHC |
| 27234 | FQIRFDRHD |
| 27235 | FQIRFDRHR |
| 27236 | FQIRFDRNR |
| 27237 | FQIRFDRPH |
| 27238 | FQIRFDRRQ |
| 27239 | FQIRFDRYH |
| 27240 | FQIRFGLHH |
| 27241 | FQIRFHRHH |
| 27242 | FQIRFNRHH |
| 27243 | FQIRFYRHH |
| 27244 | FQLRFDRHH |
| 27245 | FQMRFDRHQ |
| 27246 | FQSRFDRHH |
| 27247 | FQSRFDRHR |
| 27248 | FQTRFDRHH |
| 27249 | FQVRFDRHH |
| 27250 | FRIRFDRHH |
| 27251 | FRIRFDRHY |
| 27252 | FSAGALAYN |
| 27253 | FSAGGLAYN |
| 27254 | FSAGPLAYC |
| 27255 | FSAGPLAYY |
| 27256 | FSAGPLDYN |
| 27257 | FSAGPLSYI |
| 27258 | FSAGPLVYN |
| 27259 | FSAGPMDYN |
| 27260 | FSAGSLAYN |
| 27261 | FSAGTLAYS |
| 27262 | FSDGPLAYN |
| 27263 | FSIRFDRHH |
| 27264 | FSPGPLAYN |
| 27265 | FSTGPLSYN |
| 27266 | FSVGPLAYN |
| 27267 | FTPIVKRHH |
| 27268 | FTPQKDNSC |
| 27269 | FTPQKDTSC |
| 27270 | FTQEKDTSC |
| 27271 | FTQQKDASC |
| 27272 | FVAANQNNM |
| 27273 | GAHNELVGY |
| 27274 | GASRQAVWA |
| 27275 | GAYLYNIPM |
| 27276 | GAYLYNLPM |
| 27277 | GCCSDATNN |
| 27278 | GEVVVCILR |
| 27279 | GFAADSKPC |
| 27280 | GFNHIAWWQ |
| 27281 | GFTGMSDQD |
| 27282 | GFYLTREKK |
| 27283 | GGGQVEIGW |
| 27284 | GIANDDDRE |
| 27285 | GIANDDDWA |
| 27286 | GIANDDDWE |
| 27287 | GIANLDDWE |
| 27288 | GIANVADWA |
| 27289 | GIANVADWE |
| 27290 | GIANVDAWV |
| 27291 | GIANVDDLE |
| 27292 | GIANVDDWK |
| 27293 | GIANVDDWR |
| 27294 | GIANVDDWT |
| 27295 | GIANVDEWK |
| 27296 | GIANVDHWE |
| 27297 | GIANVDNWE |
| 27298 | GIANVDYWE |
| 27299 | GIANVEAWE |
| 27300 | GIANVGDWG |
| 27301 | GIDNFDDWE |
| 27302 | GIDNVDDLE |
| 27303 | GIDNVDDWG |
| 27304 | GIDNVDEWE |
| 27305 | GIDNVDGWE |
| 27306 | GIDNVDYWE |
| 27307 | GIGNVDDWE |
| 27308 | GIPNVDGWE |
| 27309 | GISNVDDWE |
| 27310 | GISNVDDWG |
| 27311 | GISRQAVWA |
| 27312 | GITNVDDWE |
| 27313 | GITNVDGWE |
| 27314 | GIVNVDDWE |
| 27315 | GIWRSREHY |
| 27316 | GIWRTREHH |
| 27317 | GKANDDDWE |
| 27318 | GKANVDDRE |
| 27319 | GLPNVDDWE |
| 27320 | GNWRTREHY |
| 27321 | GRANVDDCE |
| 27322 | GRCNDAWNM |
| 27323 | GSHARPLNM |
| 27324 | GSHVGPLNM |
| 27325 | GSHVGTLNM |
| 27326 | GSHVQPLNM |
| 27327 | GSHVRPLHM |
| 27328 | GSHVRPLSM |
| 27329 | GSHVRSLNM |
| 27330 | GTANVDDLE |
| 27331 | GTHNELVGY |
| 27332 | GTSRQALWA |
| 27333 | GTSRQAVWS |
| 27334 | GTSRRAVWA |
| 27335 | GVTDLQNIC |
| 27336 | GVVNVDDWE |
| 27337 | GVYRSMFQE |
| 27338 | GWAADAKPC |
| 27339 | GWAADSEHC |
| 27340 | GWAADSEPC |
| 27341 | GWAADSKAC |
| 27342 | GWAADSKHC |
| 27343 | GWAADSKLC |
| 27344 | GWAADSKRC |
| 27345 | GWAADSKSC |
| 27346 | GWAADSKTC |
| 27347 | GWAADSRPC |
| 27348 | GWAADSTPC |
| 27349 | GWAADTKPC |
| 27350 | GWAAEAKPC |
| 27351 | GWAAGSTPC |
| 27352 | GWAANSKPC |
| 27353 | GWADDSKPC |
| 27354 | GWADDSKTC |
| 27355 | GWAGDSKPC |
| 27356 | GWASDSKPC |
| 27357 | GWATDSKPC |
| 27358 | GWAVDSKPC |
| 27359 | GWAVNSKPC |
| 27360 | GWDDDSKPC |
| 27361 | GWGVDSKPC |
| 27362 | GWHQKCMGD |
| 27363 | GWSADSKPC |
| 27364 | GWTADSKPC |
| 27365 | GWYKKCMGD |
| 27366 | GWYQKCMGN |
| 27367 | GWYQKCVGD |
| 27368 | GWYQKYMGD |
| 27369 | HFNINIYNC |
| 27370 | HFYVSGFLI |
| 27371 | HGSRNWFAC |
| 27372 | HLACLKMEM |
| 27373 | HLDCLKMQM |
| 27374 | HLDCLQMEM |
| 27375 | HLDCLRMEM |
| 27376 | HLNCLKMEM |
| 27377 | HMDCLKMEM |
| 27378 | HQDCLKMEM |
| 27379 | HWRMACVHC |
| 27380 | HWRMGCVHC |
| 27381 | HWRMVCVHG |
| 27382 | HWRMVCVYC |
| 27383 | IRDWQAYWL |
| 27384 | IRDWQCYWL |
| 27385 | IRDWQGHWL |
| 27386 | IWGIYVHYC |
| 27387 | IWGIYVLYF |
| 27388 | IWGIYVYYF |
| 27389 | KAGCFVDVW |
| 27390 | KARFHRERS |
| 27391 | KDLDVWQTS |
| 27392 | KDROIDQYH |
| 27393 | KENNTNRID |
| 27394 | KGAENIHWG |
| 27395 | KGAPWAYNC |
| 27396 | KGDAYIHWG |
| 27397 | KGDENIHWC |
| 27398 | KGDEYIHWA |
| 27399 | KGDEYIHWC |
| 27400 | KGDEYIQGG |
| 27401 | KGDEYIRWG |
| 27402 | KGDEYIYWG |
| 27403 | KGDEYVHWG |
| 27404 | KGDGYIHWG |
| 27405 | KGDVHIHWG |
| 27406 | KGDVYIHWG |
| 27407 | KGGAYIHWG |
| 27408 | KGGCFVDVW |
| 27409 | KGGEYIHWC |
| 27410 | KGGLWAYNC |
| 27411 | KGGPLAYNC |
| 27412 | KGGPWAHNC |
| 27413 | KGGPWGYNC |
| 27414 | KGGQWAYNC |
| 27415 | KGGRWAYNC |
| 27416 | KGGVYIHWG |
| 27417 | KGHEYIHWG |
| 27418 | KGNEYIHWG |
| 27419 | KGPEYIHWG |
| 27420 | KGTEYIHWG |
| 27421 | KGVAYIHWG |
| 27422 | KPRFHRERS |
| 27423 | KSGRGKLTW |
| 27424 | KSRLGKLTW |
| 27425 | KSRRAKLTW |
| 27426 | KSRRGKLAW |
| 27427 | KSRRGKLKW |
| 27428 | KSRRGKLPL |
| 27429 | KSRRGKLPW |
| 27430 | KSRRGKLRW |
| 27431 | KSRRGKLSW |
| 27432 | KSRRGKLTL |
| 27433 | KSRRGKMTW |
| 27434 | KSRRGKRTW |
| 27435 | KSRRGKVTW |
| 27436 | KSRRVKLTW |
| 27437 | KSWGWVDTA |
| 27438 | KTGHFNQRG |
| 27439 | KTRFHREPS |
| 27440 | KTRFHRERN |
| 27441 | KTRFHRERT |
| 27442 | KTRRGRLTW |
| 27443 | KTWFHRERS |
| 27444 | KVGCSVDVW |
| 27445 | KVGCVVDVW |
| 27446 | LEKNGSQMR |
| 27447 | LEKNSAQMR |
| 27448 | LEKNSSEMR |
| 27449 | LEKNSSKMR |
| 27450 | LEKNSSLMR |
| 27451 | LEKNSSPMR |
| 27452 | LEKNSSQMK |
| 27453 | LEKNSSQTR |
| 27454 | LEKNSSQVR |
| 27455 | LEKNTSQMR |
| 27456 | LEQNSSQMR |
| 27457 | LERNSSQMR |
| 27458 | LLDCLKMEM |
| 27459 | LMENHEFQD |
| 27460 | LQDNINYSY |
| 27461 | LYLPLCHRI |
| 27462 | MASIDKQNV |
| 27463 | MCTGELDGT |
| 27464 | MCTGELSGT |
| 27465 | MCTGEWNGT |
| 27466 | MCTGKLNGT |
| 27467 | MCTRELNGT |
| 27468 | MEKNSSQMR |
| 27469 | MEMTQEWMA |
| 27470 | MEQNLQLTM |
| 27471 | MFTGELNGT |
| 27472 | MICGTHDNV |
| 27473 | MIFGTHDHV |
| 27474 | MIFGTHDNF |
| 27475 | MIFGTHDNI |
| 27476 | MIIGTHDNV |
| 27477 | MMVTKEFNI |
| 27478 | MMVTKEFNL |
| 27479 | MNLCYSSMM |
| 27480 | MQIRFDRHH |
| 27481 | MQMAQEWMA |
| 27482 | MQMMQMCVP |
| 27483 | MQMTQEWMG |
| 27484 | MQMTQKWMA |
| 27485 | MTAYFKALC |
| 27486 | MWTGELNGT |
| 27487 | NDLDVWQTS |
| 27488 | NELLWCEVQ |
| 27489 | NEMLWCEVQ |
| 27490 | NFCHSGFLI |
| 27491 | NFDLSGFLI |
| 27492 | NFEMWDWFG |
| 27493 | NFHINIYNC |
| 27494 | NFHLSGFLI |
| 27495 | NFNIHIYNC |
| 27496 | NFNINIFNC |
| 27497 | NFNINISNC |
| 27498 | NFNINTYNC |
| 27499 | NFNINVYNC |
| 27500 | NFNITIYNC |
| 27501 | NFNLSGFLI |
| 27502 | NFNMNIYNC |
| 27503 | NFSINIYNC |
| 27504 | NFSTNIYNC |
| 27505 | NFTINIYNC |
| 27506 | NFYCSGFLI |
| 27507 | NFYFSGFLL |
| 27508 | NFYHSGFLI |
| 27509 | NFYISGFLI |
| 27510 | NFYLGGFLI |
| 27511 | NFYLIGFLI |
| 27512 | NFYLSGFLM |
| 27513 | NFYLSGFVI |
| 27514 | NFYLTGFLI |
| 27515 | NFYLTGYLI |
| 27516 | NFYVSGFLI |
| 27517 | NKIDVQWLQ |
| 27518 | NMECEKFLT |
| 27519 | NMECERFLT |
| 27520 | NMECQQFLT |
| 27521 | NQIDVQWIQ |
| 27522 | NQIDVQWLK |
| 27523 | NQLDVQWLQ |
| 27524 | NQPCINTYW |
| 27525 | NQPCVNTCW |
| 27526 | NSRRGRLTW |
| 27527 | NVMMPGLKV |
| 27528 | PELLWCEVQ |
| 27529 | PEVMVCILR |
| 27530 | PIFGWMNWF |
| 27531 | PKYAWSTSP |
| 27532 | PKYHWHMYH |
| 27533 | PRLQIRKYL |
| 27534 | PSCVTQESI |
| 27535 | PSTTVMNVM |
| 27536 | PTFGWMDWF |
| 27537 | PTFGWMNLF |
| 27538 | PTFGWMNWV |
| 27539 | PTFGWMNWY |
| 27540 | PTFVWMNWF |
| 27541 | PTYGWMNWF |
| 27542 | PWGDMKDPQ |
| 27543 | PWGDMKDSH |
| 27544 | PWGVMKDPH |
| 27545 | PWGYMKDPH |
| 27546 | QAYLYNIPM |
| 27547 | QCRLHMQCP |
| 27548 | QCRQSHEQE |
| 27549 | QCSRNWFAC |
| 27550 | QDSRNWFAC |
| 27551 | QGGRNWFAC |
| 27552 | QGSGNWFAC |
| 27553 | QGSRNWFGC |
| 27554 | QGSRNWFSC |
| 27555 | QGSRNWYAC |
| 27556 | QGTRNWFAC |
| 27557 | QIGRFAMAH |
| 27558 | QIGRFAMPH |
| 27559 | QIGRFAMYH |
| 27560 | QIGRFTMSH |
| 27561 | QNSRHRYAW |
| 27562 | QSRRGKLTW |
| 27563 | QSSRNWFAC |
| 27564 | QTLGWAMGT |
| 27565 | RAEGWFKYD |
| 27566 | RAHNGLVGY |
| 27567 | RANMCDCFE |
| 27568 | RANNWTRRH |
| 27569 | RASICDYFE |
| 27570 | RASMCDFFE |
| 27571 | RASMCDGFE |
| 27572 | RASMCDYFE |
| 27573 | RASMWDCFE |
| 27574 | RCHNELVGY |
| 27575 | RDSMCDCFE |
| 27576 | RDVIVCILR |
| 27577 | REAMVCILR |
| 27578 | REDMACILR |
| 27579 | REDMGCILR |
| 27580 | REDMLCILR |
| 27581 | REDMVCMLR |
| 27582 | REDMVGILR |
| 27583 | REDTVCILR |
| 27584 | REFMVCILR |
| 27585 | REGMLCILR |
| 27586 | REGMVCILR |
| 27587 | REGVVCILR |
| 27588 | REIMVCILR |
| 27589 | RELMVCILR |
| 27590 | RETMVCILR |
| 27591 | REVKVCILR |
| 27592 | REVLVCILK |
| 27593 | REVMACILR |
| 27594 | REVMCCILR |
| 27595 | REVMFCILR |
| 27596 | REVMGCILR |
| 27597 | REVMGCMLR |
| 27598 | REVMICILR |
| 27599 | REVMLCILR |
| 27600 | REVMVCIII |
| 27601 | REVMVCIIK |
| 27602 | REVMVCIIR |
| 27603 | REVMVCIIT |
| 27604 | REVMVCILG |
| 27605 | REVMVCILK |
| 27606 | REVMVCIPR |
| 27607 | REVMVCIRR |
| 27608 | REVMVCIVI |
| 27609 | REVMVCIVR |
| 27610 | REVMVCLLR |
| 27611 | REVMVCTLR |
| 27612 | REVMVCVLK |
| 27613 | REVMVCVLR |
| 27614 | RFIGMSDQD |
| 27615 | RFNGMSDQD |
| 27616 | RFPGMSDQD |
| 27617 | RFTGMGDQD |
| 27618 | RFTGMNDQD |
| 27619 | RFTGMSAQD |
| 27620 | RFTGMSDED |
| 27621 | RFTGMSDKD |
| 27622 | RFTGMSDKV |
| 27623 | RFTGMSDQY |
| 27624 | RFTGMSDRD |
| 27625 | RFTGMSGQD |
| 27626 | RFTGMSHQD |
| 27627 | RFTGMSYQD |
| 27628 | RFTGMTDQD |
| 27629 | RFTGVSDQD |
| 27630 | RGHNGKGPM |
| 27631 | RGSMCDCFE |
| 27632 | RGVMQQMNH |
| 27633 | RIANVDEWE |
| 27634 | RIHNELVGY |
| 27635 | RIPNVDDWE |
| 27636 | RLDCLKMEM |
| 27637 | RMSGMLCTV |
| 27638 | RMSGMLFTL |
| 27639 | RMSGMVCTL |
| 27640 | RNHNEIVGY |
| 27641 | RNHNELVGC |
| 27642 | RNHNELVGY |
| 27643 | RNSRHKYAW |
| 27644 | RPDLNEIVC |
| 27645 | RPDLNEVEC |
| 27646 | RPGLNEIEC |
| 27647 | RPHNELVGY |
| 27648 | RPHNGLVGY |
| 27649 | RSHNGLVGY |
| 27650 | RSHVQPLNM |
| 27651 | RSRRGKLTW |
| 27652 | RSRRGKMTW |
| 27653 | RSSMCDCFE |
| 27654 | RSWRGKLTW |
| 27655 | RTDNEIVGY |
| 27656 | RTDNELVGD |
| 27657 | RTDNGLVGY |
| 27658 | RTEGWFEYD |
| 27659 | RTEGWFKSD |
| 27660 | RTEGWFKYA |
| 27661 | RTEGWFKYE |
| 27662 | RTEGWVKYD |
| 27663 | RTHIELVGY |
| 27664 | RTHNALVCY |
| 27665 | RTHNALVSY |
| 27666 | RTHNALVVY |
| 27667 | RTHNDLVGH |
| 27668 | RTHNDLVVY |
| 27669 | RTHNEFVGY |
| 27670 | RTHNEIFGY |
| 27671 | RTHNEIIGY |
| 27672 | RTHNEIVGF |
| 27673 | RTHNEIVGS |
| 27674 | RTHNEIVGY |
| 27675 | RTHNELFGH |
| 27676 | RTHNELFGS |
| 27677 | RTHNELFGY |
| 27678 | RTHNELGGY |
| 27679 | RTHNELHGY |
| 27680 | RTHNELIGY |
| 27681 | RTHNELLCY |
| 27682 | RTHNELLGF |
| 27683 | RTHNELVAH |
| 27684 | RTHNELVCD |
| 27685 | RTHNELVCH |
| 27686 | RTHNELVCS |
| 27687 | RTHNELVCY |
| 27688 | RTHNELVFY |
| 27689 | RTHNELVGE |
| 27690 | RTHNELVGF |
| 27691 | RTHNELVGG |
| 27692 | RTHNELVGH |
| 27693 | RTHNELVGQ |
| 27694 | RTHNELVGV |
| 27695 | RTHNELVIY |
| 27696 | RTHNELVRH |
| 27697 | RTHNELVRY |
| 27698 | RTHNELVSF |
| 27699 | RTHNELVSY |
| 27700 | RTHNELVVF |
| 27701 | RTHNELVVY |
| 27702 | RTHNEPVGY |
| 27703 | RTHNEVVGC |
| 27704 | RTHNEVVGD |
| 27705 | RTHNEVVGS |
| 27706 | RTHNGLVGF |
| 27707 | RTHNGLVGS |
| 27708 | RTHNKLGGY |
| 27709 | RTHNKLVGN |
| 27710 | RTHNKLVGY |
| 27711 | RTHNQLVGC |
| 27712 | RTHNQLVGY |
| 27713 | RTHNVLVGS |
| 27714 | RTHNWLVGY |
| 27715 | RTHSELVGY |
| 27716 | RTHSQLVGY |
| 27717 | RTHTKLVGY |
| 27718 | RTMGQFYYE |
| 27719 | RTNNEIVGY |
| 27720 | RTNNELAGY |
| 27721 | RTNNELIGY |
| 27722 | RTNNELVGS |
| 27723 | RTPNALVGY |
| 27724 | RTQGWFKYD |
| 27725 | RTQNELVGS |
| 27726 | RTQNELVVY |
| 27727 | RTQNGLVGY |
| 27728 | RTRNELFGY |
| 27729 | RTRNELLGY |
| 27730 | RTRNELVGY |
| 27731 | RTRNELVVY |
| 27732 | RTRNKLVGY |
| 27733 | RTRSELVGY |
| 27734 | RTYNELVGY |
| 27735 | RVGMQQMNH |
| 27736 | RVMAEMVWE |
| 27737 | RVSGMLCTL |
| 27738 | RVVMQPMNH |
| 27739 | RVVMVCILR |
| 27740 | RWAADSKPC |
| 27741 | RWDNGKGPM |
| 27742 | RWGDMKDPH |
| 27743 | RWHNGKAPM |
| 27744 | RWHNGKEPM |
| 27745 | RWHNGKGHM |
| 27746 | RWHNGKGPK |
| 27747 | RWHNGKGRM |
| 27748 | RWHNGKGSM |
| 27749 | RWHNGKVPM |
| 27750 | RWHNGQGPM |
| 27751 | RWHNGRGPM |
| 27752 | RWHTGKGPM |
| 27753 | RWNNGKGPM |
| 27754 | RWRMVCVHC |
| 27755 | RYGNVAHHA |
| 27756 | RYSNVAHYA |
| 27757 | RYTGMSDQD |
| 27758 | SATNWECSK |
| 27759 | SCCADATNN |
| 27760 | SCCSDAANK |
| 27761 | SCCSDAANN |
| 27762 | SCCSDANNN |
| 27763 | SCCSDATDN |
| 27764 | SCCSDATNH |
| 27765 | SCCSDATQN |
| 27766 | SCCSDATSN |
| 27767 | SCCYDATNN |
| 27768 | SCTTRVWIC |
| 27769 | SCWSDATNN |
| 27770 | SCYSDATNN |
| 27771 | SEVMVCVLR |
| 27772 | SEYHWHMYH |
| 27773 | SFCSDATNN |
| 27774 | SFCSDATNS |
| 27775 | SFYLSGFLI |
| 27776 | SKCHWHMYH |
| 27777 | SKFAWSTSP |
| 27778 | SKFHWHMYH |
| 27779 | SKYAWSTAP |
| 27780 | SKYAWSTSQ |
| 27781 | SKYAWSTSS |
| 27782 | SKYAWSTTP |
| 27783 | SKYDWSTSP |
| 27784 | SKYGWSTSP |
| 27785 | SKYHWDMYH |
| 27786 | SKYHWHIYP |
| 27787 | SKYHWHMYL |
| 27788 | SKYHWHMYR |
| 27789 | SKYHWNMYH |
| 27790 | SKYHWPMYH |
| 27791 | SKYHWQIYH |
| 27792 | SKYHWRMYH |
| 27793 | SKYLWHMYH |
| 27794 | SKYNWHMYH |
| 27795 | SKYNWHMYQ |
| 27796 | SKYPWSTSP |
| 27797 | SKYQWHMYH |
| 27798 | SKYRWHMYH |
| 27799 | SKYSWSTSP |
| 27800 | SKYTWSTSP |
| 27801 | SKYVWSTSP |
| 27802 | SKYYWHMYH |
| 27803 | SPTNWECSN |
| 27804 | SRYAWSTSQ |
| 27805 | SRYHWHMYH |
| 27806 | STFGWMNWF |
| 27807 | STMTDAPCG |
| 27808 | STTNWECSN |
| 27809 | SVCSDATNN |
| 27810 | SVMMSPHLM |
| 27811 | SWAADSKPC |
| 27812 | SYARKAYDC |
| 27813 | SYPRRVYDC |
| 27814 | TALLWCEVL |
| 27815 | TAYMTAMYC |
| 27816 | TCCSDATNN |
| 27817 | TDLLWCELQ |
| 27818 | TDLLWCEVH |
| 27819 | TDRQIAQYH |
| 27820 | TDRQIDQYL |
| 27821 | TDRQIDQYR |
| 27822 | TDRQIGQYH |
| 27823 | TDRQIHQYH |
| 27824 | TDRQVDQYH |
| 27825 | TELFWCDVQ |
| 27826 | TELFWCEVL |
| 27827 | TELLLCEVH |
| 27828 | TELLWCAVQ |
| 27829 | TELLWCEIK |
| 27830 | TELLWCEIQ |
| 27831 | TELLWCELQ |
| 27832 | TELLWCELR |
| 27833 | TELLWCEVA |
| 27834 | TELLWCEVE |
| 27835 | TELLWCEVG |
| 27836 | TELLWCGVQ |
| 27837 | TELLWCKVQ |
| 27838 | TELLWCQVQ |
| 27839 | TELLWCVVQ |
| 27840 | TELLWFEVQ |
| 27841 | TELLWGEVQ |
| 27842 | TELLWREVQ |
| 27843 | TELLWSEIQ |
| 27844 | TELSWCEVQ |
| 27845 | TELVWCEVK |
| 27846 | TEMLWCEVK |
| 27847 | TEMLWCEVL |
| 27848 | TEMLWCEVQ |
| 27849 | TERLWCEVL |
| 27850 | TFNINIYNC |
| 27851 | TFWMPDDNC |
| 27852 | TFWMPGNNC |
| 27853 | TFYLSGFLI |
| 27854 | TGLLWCEVQ |
| 27855 | TISHDEAHK |
| 27856 | TKLLWCEVK |
| 27857 | TKYAWSTSP |
| 27858 | TKYHWHMYH |
| 27859 | TMGEVVINL |
| 27860 | TMSEVVINL |
| 27861 | TNGCAIENH |
| 27862 | TPGIHRHCC |
| 27863 | TPGISRHCC |
| 27864 | TPGITRHCC |
| 27865 | TPGIYRHCC |
| 27866 | TPGLNRHCC |
| 27867 | TOLLWCEVQ |
| 27868 | TRRSWECSP |
| 27869 | TRYRMPYHG |
| 27870 | TRYRMRYHC |
| 27871 | TRYRMRYHS |
| 27872 | TRYRMRYNG |
| 27873 | TSCGYHDNN |
| 27874 | TSCGYNDNS |
| 27875 | TSCRYNDNN |
| 27876 | TSRSAAADL |
| 27877 | TSRTEAADL |
| 27878 | TSSHDEAHR |
| 27879 | TTFGWMNWF |
| 27880 | TTGINRHCC |
| 27881 | TTMPDAPCR |
| 27882 | TTSHDEAHK |
| 27883 | TVLLWCDVQ |
| 27884 | TVLLWCEVH |
| 27885 | TVLLWCEVQ |
| 27886 | TWEIQIDCI |
| 27887 | TWETQIDCI |
| 27888 | TWGSQIDCI |
| 27889 | TWQSQIDCI |
| 27890 | TYPRNAKYT |
| 27891 | VEDKINYSY |
| 27892 | VEKNSSQMR |
| 27893 | VGYRSMFQE |
| 27894 | VIGIIRHGH |
| 27895 | VISNVDDWE |
| 27896 | VLYRSMFQE |
| 27897 | VQAQINYSY |
| 27898 | VQDEINYSY |
| 27899 | VQDKFNYSY |
| 27900 | VQDKINFSY |
| 27901 | VQDKINYGY |
| 27902 | VQDKINYNY |
| 27903 | VQDKINYSH |
| 27904 | VQDKISYSY |
| 27905 | VQDKITYSY |
| 27906 | VQDKLNYSH |
| 27907 | VQDKLNYSY |
| 27908 | VQDKRNYSY |
| 27909 | VQDKSNYSY |
| 27910 | VQDKVNYSY |
| 27911 | VQDMLNYSY |
| 27912 | VQDMVNYSY |
| 27913 | VQDNINYSH |
| 27914 | VQDQINYSY |
| 27915 | VQDRINYSY |
| 27916 | VQDRVNYSY |
| 27917 | VQDTINFSY |
| 27918 | VQDTINYSF |
| 27919 | VQDTINYSY |
| 27920 | VQDTVNYSY |
| 27921 | VQENINYSY |
| 27922 | VQGRINYSY |
| 27923 | VQGTINYSY |
| 27924 | VQNKINYSY |
| 27925 | VQVKINYSY |
| 27926 | VQVRINYSY |
| 27927 | VQVTINYSY |
| 27928 | VRDKINYSY |
| 27929 | VRDWQGYWL |
| 27930 | VSRRGKLTW |
| 27931 | VTMPDAPCG |
| 27932 | VWEANPPFR |
| 27933 | VWEDNAPFR |
| 27934 | VWEDNPPVR |
| 27935 | VWENNPPFR |
| 27936 | WEKNSSQMR |
| 27937 | WEVMVCILR |
| 27938 | WHAMVNSGP |
| 27939 | WHDMGNSGP |
| 27940 | WMCDFSCIQ |
| 27941 | WMCEFSCIK |
| 27942 | WRDMVNSGP |
| 27943 | YAEVAVQKE |
| 27944 | YANSIQMSF |
| 27945 | YAQVAVQQE |
| 27946 | YGGQVEFGW |
| 27947 | YHGVICKEL |
| 27948 | YHPHFPTYK |
| 27949 | YHPYFPAYK |
| 27950 | YHPYFPTHK |
| 27951 | YHPYFPTNK |
| 27952 | YHPYFPTYQ |
| 27953 | YHPYFPTYR |
| 27954 | YHPYFSTYK |
| 27955 | YHPYVPTYR |
| 27956 | YIANPCANC |
| 27957 | YIDNPCGNC |
| 27958 | YIDNPCTNC |
| 27959 | YIDTPCANC |
| 27960 | YIHNPCANC |
| 27961 | YKLNRDDSW |
| 27962 | YKMDCHSCG |
| 27963 | YKVNGDDSW |
| 27964 | YKVNLDDSW |
| 27965 | YKVNQDDSW |
| 27966 | YKVNRDASW |
| 27967 | YKVNRDGSW |
| 27968 | YKVNRDVSW |
| 27969 | YKVNRDYSW |
| 27970 | YKVNRNDSW |
| 27971 | YNGGICKEL |
| 27972 | YNGVFCKEL |
| 27973 | YNGVICKVL |
| 27974 | YNGVICQEL |
| 27975 | YNGVICREL |
| 27976 | YNGVIGKEL |
| 27977 | YNGVLCKEL |
| 27978 | YNGVMCKEL |
| 27979 | YNGVTCKEL |
| 27980 | YNGVVCKEL |
| 27981 | YQIRFDRHH |
| 27982 | YRVNRDDSW |
| 27983 | YSAGPLAYN |
| 27984 | YSNSIKMSL |
| 27985 | YSNSVQMSF |
| 27986 | YSNTIQMSL |
| 27987 | YTGVICQEL |
| 27988 | YWRMVCVHC |
| 27989 | YYMHWINSG |
| 27990 | YYMHWVNAG |

### Example 39

AAV5 Variants with Skeletal Muscle Tissue Tropism as Identified by Machine Learning

This example describes preferred properties of engineered AAV5 variants that display skeletal muscle tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display skeletal muscle tissue tropism.

Favored biophysical properties and favored amino acid residues at each position in the 581 to 589 region of an AAV5 VP capsid polypeptide, which are associated with a higher probability of skeletal muscle tissue tropism are described below. Any of the below machine learning-derived positional amino acid preferences described in TABLE 62 can be present alone or in combination with each other or in combination with any of the amino acid preferences observed and described in EXAMPLE 9, TABLE 17. Listed below in TABLE 62 are a summary of positional features shared between the top important features for skeletal muscle tropism extracted from the ML models.

TABLE 62
Machine Learning-Derived Skeletal Muscle Tissue Tropism Rules

High average flexibility at Xaa1
Xaa1 is selected from G or R
Or
Low average flexibility at Xaa1
Xaa1 is selected from W, M, F, or H
High mol mass at Xaa1
Xaa1 is selected from R, F, or W
Low hydropathy at Xaa2
Xaa2 is selected from K or R
Low mutability at Xaa2
Xaa2 is selected from C, R, or H
High average flexibility at Xaa2
Xaa2 is selected from G or R
High average flexibility at Xaa3
Xaa3 is selected from G or R
High hydrophilicity at Xaa4
Xaa4 is selected from D, E, R, K, or N
Low mutability at Xaa4
Xaa4 is selected from C, R, or H
Low mol mass at Xaa5
Xaa5 is selected from A
Low average flexibility at Xaa5
Xaa5 is selected from A or L
High mutability at Xaa5
Xaa5 is selected from D, A, or E
Low average flexibility at Xaa6
Xaa6 is selected from W, M, or F
Low mutability at Xaa6
Xaa6 is selected from C
High mol mass at Xaa6
Xaa6 is selected from W
Low goldman engelman steitz at Xaa7
Xaa7 is selected from R
High average flexibility at Xaa7
Xaa7 is selected from D, R, P, G, or S
High mutability at Xaa7
Xaa7 is selected from R, H, or N
Low solubility at Xaa7
Xaa7 is selected from R or Q
High hydrophilicity at Xaa8
Xaa8 is selected from D, E, R, K, or N
Low mutability at Xaa9
Xaa9 is selected from Y, F, or L

TABLE 63 below provides sequences of the 581 to 589 region for variant AAV VP1 capsid polypeptides that exhibited skeletal muscle tissue tropism and comport to one or more of the rules provided in TABLE 62. The present disclosure, thus, provides for rAAVs composed of engineered AAV5 VP1 capsid polypeptides having a SEQ ID NO: 2, wherein the Xaa1 to Xaa9 region is any one of SEQ ID NO: 29991-SEQ ID NO: 30990, as disclosed in TABLE 63. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 63
Sequences of the 581 to 589 Region in AAV5 VP1
Capsid Polypeptide that Drive Skeletal
Muscle Tissue Tropism

| SEQ | 581-589 |
|---|---|
| ID NO | Sequence |
| 29991 | AAGDIGSVM |
| 29992 | AAGNIGSVM |
| 29993 | AANLLWRLW |
| 29994 | AANLLWRLY |
| 29995 | ACAWIESFT |
| 29996 | ACPDHHRWL |
| 29997 | ACPNVWWHM |
| 29998 | ACQCKVIGQ |
| 29999 | ACQCRVIWQ |
| 30000 | ACTWPGVGQ |
| 30001 | ACWDTHQSI |
| 30002 | ACWDTKQSI |
| 30003 | ACWDTLQSI |
| 30004 | ACWDTQKSI |
| 30005 | ACWDTQQGI |
| 30006 | ACWRHVQWN |
| 30007 | AFGEFDNHS |
| 30008 | AFIHIDREC |
| 30009 | AGGHCTYVA |
| 30010 | AGWIGACMA |
| 30011 | AHWNAIMRW |
| 30012 | AIFEPHRWH |
| 30013 | AIFEPRRWP |
| 30014 | AIIEPRRWH |
| 30015 | AIKWRSQDK |
| 30016 | AIRVDDSTI |
| 30017 | AIVSMDDWH |
| 30018 | AKREIENCH |
| 30019 | AMHKRWTND |
| 30020 | ANNVYCVKK |
| 30021 | APANTPWRK |
| 30022 | APRPPRSAR |
| 30023 | ARDNESNLI |
| 30024 | ARSGAQSMM |
| 30025 | ARSGFASNT |
| 30026 | ARSSFASNN |
| 30027 | ARSSFASNS |
| 30028 | ARSSFPSNT |
| 30029 | ARSSFSSNT |
| 30030 | ARSSFTSNT |
| 30031 | ARSSVASNT |
| 30032 | ASTEVFYMI |
| 30033 | ATGIDNNAT |
| 30034 | AWSCVHADV |
| 30035 | AWWARCKQQ |
| 30036 | AWWTPCKQQ |
| 30037 | AWWTRCKQL |
| 30038 | CADVRENAN |
| 30039 | CCEIWLASA |
| 30040 | CCELWLASS |
| 30041 | CCRHWRPTT |
| 30042 | CCRYWRPTP |
| 30043 | CGGKIDIGL |
| 30044 | CHQENFIFR |
| 30045 | CHQENFVFH |
| 30046 | CHQENFVFP |
| 30047 | CIETGLLNG |
| 30048 | CIRQYDAIH |
| 30049 | CITYEGWAD |
| 30050 | CKVSESAKN |
| 30051 | CPPICEGGT |
| 30052 | CTETTVRWP |
| 30053 | CTKTTVRWP |
| 30054 | CTPTTVRWP |
| 30055 | CTQSSVRWP |
| 30056 | CTQTAVRWP |
| 30057 | CTQTTIRWP |
| 30058 | CTQTTVRWR |
| 30059 | CTQTTVRYP |
| 30060 | CTRSTVRWP |
| 30061 | CTRTTVRWP |
| 30062 | CVGMDSYNG |
| 30063 | CYGHLTAMC |
| 30064 | DARASPDCL |
| 30065 | DARPQWDIP |
| 30066 | DARSSPDCI |
| 30067 | DCEEFDRTT |
| 30068 | DCLVQKAAL |
| 30069 | DCNVNPACF |
| 30070 | DCWAIHSSW |
| 30071 | DCWDTQQSI |
| 30072 | DEVHGFLSG |
| 30073 | DEVYSFLSG |
| 30074 | DFHSYCMSF |
| 30075 | DFIHVGDWE |
| 30076 | DFTHVGDWD |
| 30077 | DGDPNFRIQ |
| 30078 | DHGYRNNSM |
| 30079 | DILIDHDFF |
| 30080 | DKQHDCRAS |
| 30081 | DKQQDCRAY |
| 30082 | DQQHDCRAY |
| 30083 | DRSSLCADE |
| 30084 | DRYSLCADA |
| 30085 | DSGRIYAWI |
| 30086 | DSNVQFCID |
| 30087 | DSRSSPDCL |
| 30088 | DSTLQFCID |
| 30089 | DTRKWMKWH |
| 30090 | DVPIDHDFF |
| 30091 | DVVGDAACP |
| 30092 | DWWTRCKQQ |
| 30093 | EARLQWDIP |
| 30094 | EARPHWDIP |
| 30095 | EARPQWDIL |
| 30096 | EARPQWDIS |
| 30097 | EARPQWDIT |
| 30098 | EARPQWDLP |
| 30099 | EARPQWDSP |
| 30100 | EARPQWDVP |
| 30101 | ECWQHVQWN |
| 30102 | ECWRHVKWN |
| 30103 | ECWRHVQLN |
| 30104 | ECWRHVQWI |
| 30105 | ECWRHVQWK |
| 30106 | ECWRHVQWS |
| 30107 | ECWRQVQWN |
| 30108 | ECYRKVVFR |
| 30109 | ECYSIYCEQ |
| 30110 | EDNIRMMMI |
| 30111 | EFAMDWVSD |
| 30112 | EFWGSMRNG |
| 30113 | EFWSSMRTG |
| 30114 | EGRPQWDIP |
| 30115 | EILAAWTPP |
| 30116 | EKCFQWTSP |
| 30117 | EKCFQWTSS |
| 30118 | EKCYQWTSA |
| 30119 | EKDLSHATF |
| 30120 | EKDLSQDTF |
| 30121 | EKTSRHWKT |
| 30122 | EKVHCMWLH |
| 30123 | EQEGDYVDM |
| 30124 | EQGAVWTPP |
| 30125 | EQGTVWTPP |
| 30126 | EQVRAFRQI |
| 30127 | ERAGFKMMD |
| 30128 | ERAGFQMMH |
| 30129 | ERAGVKMMH |
| 30130 | ERAGYKMMH |
| 30131 | ERASFKMMH |
| 30132 | ERDGFKMMH |
| 30133 | ERGGFKMMH |
| 30134 | ERVGFKMMH |
| 30135 | ESRPQWDIP |
| 30136 | ETCEMFTPG |
| 30137 | ETGNDNNAT |
| 30138 | ETPINSEKG |
| 30139 | EVGTDPSKN |
| 30140 | EVRPQWDIP |
| 30141 | FAGKIDIGL |
| 30142 | FAGPNSYWP |
| 30143 | FAMFASPDD |
| 30144 | FAVMIHYHN |
| 30145 | FAVMVHYPN |
| 30146 | FAVMVRYHN |
| 30147 | FCAAEWGCL |
| 30148 | FCPAEWACL |
| 30149 | FCPAEWGCV |
| 30150 | FCPAEWGGL |
| 30151 | FCPAEWGYL |
| 30152 | FCPAKWGCL |
| 30153 | FCPEHWFDI |
| 30154 | FCPEHWFNM |
| 30155 | FCPEHWENN |
| 30156 | FCPEHWFNS |
| 30157 | FCPEHWLNI |
| 30158 | FCPEHWVNI |
| 30159 | FCPQHWFNI |
| 30160 | FCSAEWGCL |
| 30161 | FCTAEWGCL |
| 30162 | FCTEHWFNI |
| 30163 | FDLFASPDD |
| 30164 | FDMFASPAD |
| 30165 | FDMFASPDA |
| 30166 | FDMFASPGD |
| 30167 | FDVFASPDD |
| 30168 | FFELSKTVF |
| 30169 | FFELSQTEF |
| 30170 | FFRDCPYES |
| 30171 | FFRDYPYEY |
| 30172 | FGGKFDIGL |
| 30173 | FGGKIAIGL |
| 30174 | FGGKIDIGV |
| 30175 | FGGKIDIVL |
| 30176 | FGGKIDVGL |
| 30177 | FGGKIHIGL |
| 30178 | FGGKINIGL |
| 30179 | FGGKLDIGL |
| 30180 | FGGTIDIGL |
| 30181 | FGLVHQKNA |
| 30182 | FGRKIDIGL |
| 30183 | FGVVHQKDA |
| 30184 | FHHLTEYHG |
| 30185 | FHHQPEYHG |
| 30186 | FHHQTEYRG |
| 30187 | FHHQTGYHG |
| 30188 | FHHQTQYHG |
| 30189 | FHKWGINLG |
| 30190 | FHKWWIHLG |
| 30191 | FHKWWINLA |
| 30192 | FHKWWINLS |
| 30193 | FHKWWINQG |
| 30194 | FHKWWISLG |
| 30195 | FHKWWITLG |
| 30196 | FHKWWLNLG |
| 30197 | FHRQTEYHG |
| 30198 | FLKWWINLG |
| 30199 | FMGATIPED |
| 30200 | FMGVTIPAD |
| 30201 | FMGVTIPEG |
| 30202 | FMGVTIPKD |
| 30203 | FMGVTIPVD |
| 30204 | FMKCENFNS |
| 30205 | FMKCENVNT |
| 30206 | FMKCESVNS |
| 30207 | FMKGENVNS |
| 30208 | FMTCENVNS |
| 30209 | FMWHANSCG |
| 30210 | FMWHANSGA |
| 30211 | FMWYANSCA |
| 30212 | FNINAQDWD |
| 30213 | FNVTAQDWD |
| 30214 | FPHHLWVGG |
| 30215 | FPYHLWIGG |
| 30216 | FPYHLWVAG |
| 30217 | FPYHVWVGG |
| 30218 | FPYRLWVGG |
| 30219 | FRECLQIVN |
| 30220 | FRECLQLVT |
| 30221 | FRECMQIVT |
| 30222 | FREGLQIVT |
| 30223 | FRGCLQIVT |
| 30224 | FRGIETEQG |
| 30225 | FRGIETGEG |
| 30226 | FRGIETGQV |
| 30227 | FRGIETVQG |
| 30228 | FRGTETGQG |
| 30229 | FRGVETGQG |
| 30230 | FRKCLQIVT |
| 30231 | FRRWQSNTT |
| 30232 | FTETTVRWP |
| 30233 | FTQPTVRWP |
| 30234 | FVLVHQKDA |
| 30235 | FWKFALLSF |
| 30236 | FWKLALHSF |
| 30237 | FWPQIQYRM |
| 30238 | FYGKSAPVG |
| 30239 | FYGKTALVG |
| 30240 | FYGKTAPVA |
| 30241 | FYGPVTKSC |
| 30242 | FYGQLTKSC |
| 30243 | FYGQTAPVG |
| 30244 | FYGQVTESC |
| 30245 | FYGQVTMSC |
| 30246 | FYGQVTQSC |
| 30247 | FYGQVTTSC |
| 30248 | FYKWWINLG |
| 30249 | GARPQWDIP |
| 30250 | GARSSPDCL |
| 30251 | GASWHDREH |
| 30252 | GATEVFYMI |
| 30253 | GCDMDAYSN |
| 30254 | GCDVDAYSN |
| 30255 | GDCGASNCY |
| 30256 | GFHNTELPI |
| 30257 | GGAKALRFN |
| 30258 | GGARALRFI |
| 30259 | GGARALRFS |
| 30260 | GGGCEDWPM |
| 30261 | GGPEIKHYI |
| 30262 | GHWNAIMRW |
| 30263 | GIDWEDVWE |
| 30264 | GIKWRSQDK |
| 30265 | GIRDDDSTI |
| 30266 | GIRGDDSTI |
| 30267 | GIRVDDNTI |
| 30268 | GIRVDDSTK |
| 30269 | GIRVDDSTV |
| 30270 | GMDEVEREG |
| 30271 | GMEEGEREG |
| 30272 | GMEELEREG |
| 30273 | GNIYHFNNV |
| 30274 | GPDGAKLNK |
| 30275 | GPDGAKRHK |
| 30276 | GPDGARRNK |
| 30277 | GPDKCTVNL |
| 30278 | GPGLKAWPC |
| 30279 | GPLATTDPV |
| 30280 | GPTEVFYMI |
| 30281 | GRDWAFCDY |
| 30282 | GRHWACCDY |
| 30283 | GRHWAFCDD |
| 30284 | GRHWAFCDF |
| 30285 | GRHWAFCDH |
| 30286 | GRHWAFCDN |
| 30287 | GRHWAFCGY |
| 30288 | GRHWAFCHY |
| 30289 | GRHWAFFDY |
| 30290 | GRHWAFGDY |
| 30291 | GRHWAVCDY |
| 30292 | GRHWDFCDY |
| 30293 | GRHWSFCDY |
| 30294 | GRHWTFCDY |
| 30295 | GRPWAFCDY |
| 30296 | GRQWAVCDY |
| 30297 | GRRWAFCDY |
| 30298 | GRVSGHVHW |
| 30299 | GRYSLCADE |
| 30300 | GRYWAFCDY |
| 30301 | GSAEVFYMI |
| 30302 | GSNEVFYMI |
| 30303 | GSPEVFYMI |
| 30304 | GSTELFYMI |
| 30305 | GSTEVFCMI |
| 30306 | GSTEVFYMV |
| 30307 | GSTEVFYVI |
| 30308 | GSTKVFYMI |
| 30309 | GVEPMSRAA |
| 30310 | GVRDAVHWC |
| 30311 | GWSARSVNM |
| 30312 | HAISVGEWD |
| 30313 | HARGIWDFH |
| 30314 | HCAHYWMQD |
| 30315 | HCIRHFCEV |
| 30316 | HCLLOKAAL |
| 30317 | HCSCHFCEV |
| 30318 | HCSHHFCEV |
| 30319 | HCSRHFCEA |
| 30320 | HCSRHFCKV |
| 30321 | HCSRHFCQV |
| 30322 | HCSRHFGEV |
| 30323 | HCSRHYCEV |
| 30324 | HCTRHFCEV |
| 30325 | HCTRYWMQD |
| 30326 | HCYRKVVFR |
| 30327 | HDIDGWQMA |
| 30328 | HDIWQWSIK |
| 30329 | HDMLQWSIK |
| 30330 | HDMWQWTIK |
| 30331 | HDQVKCLRK |
| 30332 | HDTWQWSIK |
| 30333 | HEIDGWQTA |
| 30334 | HEIDVWQMA |
| 30335 | HEVDGWQMA |
| 30336 | HEVHSFLSG |
| 30337 | HKRNLYALQ |
| 30338 | HLHFHWGLW |
| 30339 | HLWRTFEAQ |
| 30340 | HLYSDYWFG |
| 30341 | HLYSEYWFD |
| 30342 | HRDAHLLME |
| 30343 | HRYSLCADE |
| 30344 | HSHFHWGLW |
| 30345 | HTCSEIVDG |
| 30346 | HTCSELVEG |
| 30347 | HTILSHDPW |
| 30348 | HTIWSHDQW |
| 30349 | HTLLLSREP |
| 30350 | IACMKCPNI |
| 30351 | IANNDLRFG |
| 30352 | ICEMGPYHG |
| 30353 | ICMSQEKRC |
| 30354 | IDVGAYVWS |
| 30355 | IFEQCQRMR |
| 30356 | IFRDCPYEY |
| 30357 | IGIVFQRDF |
| 30358 | IGLVFQRDF |
| 30359 | IGVVFPRDF |
| 30360 | IGVVFQRDC |
| 30361 | IGVVFQRDI |
| 30362 | IGVVFQRDV |
| 30363 | IGVVFQRDY |
| 30364 | IGVVFQRVF |
| 30365 | IHVEYFDAW |
| 30366 | IHWNAIMRW |
| 30367 | IIKWRSQDK |
| 30368 | IINKDHDKF |
| 30369 | IINKHHDRF |
| 30370 | IINKRHDKF |
| 30371 | INQMARHPK |
| 30372 | INVEYFDTW |
| 30373 | INVEYFEAW |
| 30374 | INVEYFVAW |
| 30375 | IRTSFHVNA |
| 30376 | ISNNDLRFV |
| 30377 | ISNNDLRVG |
| 30378 | ISNNDVRFG |
| 30379 | ISTNDLRFG |
| 30380 | ITCKFTQNG |
| 30381 | ITCKMDFWQ |
| 30382 | ITTGSRPTK |
| 30383 | IVFPSLARG |
| 30384 | IWPQSQYRM |
| 30385 | KAHKTVQDK |
| 30386 | KAMSVPWGK |
| 30387 | KANGSMDHH |
| 30388 | KANYACDWD |
| 30389 | KCFSMDFSG |
| 30390 | KCMPGCNDW |
| 30391 | KCMPNVGWR |
| 30392 | KCNVNPACF |
| 30393 | KCQWVGREN |
| 30394 | KCVSMAFSG |
| 30395 | KCVSMDFAG |
| 30396 | KCVSMDFTG |
| 30397 | KCVSMGFSG |
| 30398 | KCVSMVFSG |
| 30399 | KCVSMYFSG |
| 30400 | KCVSVDFSG |
| 30401 | KCWRHVQWN |
| 30402 | KDHNRWNRV |
| 30403 | KDPNRWNRL |
| 30404 | KDVGDDAHG |
| 30405 | KDYNRWNRL |
| 30406 | KEHNRWNRL |
| 30407 | KFAKDWVSD |
| 30408 | KFAMDWDSD |
| 30409 | KFAMDWVSV |
| 30410 | KFVSMDFSG |
| 30411 | KHEADYVDM |
| 30412 | KHEPRQSPR |
| 30413 | KHKARQSPR |
| 30414 | KHKLRQSPR |
| 30415 | KHMPRQSPR |
| 30416 | KHSELWREN |
| 30417 | KKCFQWTSA |
| 30418 | KKVRAFRQI |
| 30419 | KMKDVEENG |
| 30420 | KMKPVQRVG |
| 30421 | KMMSFSEVD |
| 30422 | KQEGDDVDM |
| 30423 | KQEGDSVDM |
| 30424 | KQVRAFREI |
| 30425 | KQVRAFRPI |
| 30426 | KQVRAFRQL |
| 30427 | KQVRAFRQM |
| 30428 | KQVRAFRQS |
| 30429 | KQVRAFRQV |
| 30430 | KQVRAFRRI |
| 30431 | KQVRDFRQI |
| 30432 | KQVRPFRQI |
| 30433 | KQVRSFRQI |
| 30434 | KQVRVFRQI |
| 30435 | KRVHFEGGD |
| 30436 | KSMTELDQP |
| 30437 | KTCELFTPG |
| 30438 | KTCEMFTPA |
| 30439 | KTCEMFTPS |
| 30440 | KTCERFTPG |
| 30441 | KTCGMFTPG |
| 30442 | KTELAMVID |
| 30443 | KTELDRDSM |
| 30444 | KTEWAMAID |
| 30445 | KTEWAMVLD |
| 30446 | KTEWAMVVD |
| 30447 | KTISGFQDK |
| 30448 | KTKWAMVID |
| 30449 | KTMSGPWGK |
| 30450 | KVNYACDWH |
| 30451 | KVTYACDWD |
| 30452 | KWDSIFVEH |
| 30453 | KWDSIFVQR |
| 30454 | KYAANWLKD |
| 30455 | KYADNWLKA |
| 30456 | KYADNWVKD |
| 30457 | KYADYWLKD |
| 30458 | KYSDNWLKD |
| 30459 | KYSEVWREN |
| 30460 | KYSKLWREN |
| 30461 | KYWMRHVVM |
| 30462 | LATSMCYQS |
| 30463 | LCHFLRTCK |
| 30464 | LDKIEHDWN |
| 30465 | LFCIVQSRE |
| 30466 | LIDRIWFNW |
| 30467 | LSGPIHYEP |
| 30468 | LSGSIHYES |
| 30469 | LSVKMDRAK |
| 30470 | LSVQMDRSK |
| 30471 | LSVRMDRAK |
| 30472 | LTGQEAGKG |
| 30473 | LVCIRADTD |
| 30474 | LVGKYWPRD |
| 30475 | LVGPYWPRD |
| 30476 | MADHCCANS |
| 30477 | MADHCCTDS |
| 30478 | MAFSSMHQG |
| 30479 | MASLRGSDE |
| 30480 | MCYQNIAFQ |
| 30481 | MFGECYNNG |
| 30482 | MFKYYPYCD |
| 30483 | MFLECYNNG |
| 30484 | MFQSYPYCD |
| 30485 | MFQYYQYCD |
| 30486 | MFRECYDNG |
| 30487 | MFRECYKNG |
| 30488 | MFRECYNSG |
| 30489 | MFREWYNNG |
| 30490 | MFRKCYNNG |
| 30491 | MGDGLWVKV |
| 30492 | MHFKYPQRG |
| 30493 | MNLVKIRNS |
| 30494 | MPVKKEYGG |
| 30495 | MRHWAFCDY |
| 30496 | MRIHFEGGD |
| 30497 | MRVHFEGGG |
| 30498 | MRVHFQGGD |
| 30499 | MRVRFEGGD |
| 30500 | MSSLKGSDE |
| 30501 | MSWLGAAGI |
| 30502 | MTLLMGHES |
| 30503 | MVCIHADTD |
| 30504 | MWLAKARQG |
| 30505 | MWVGKARQG |
| 30506 | MYKYGQDWY |
| 30507 | NAINVGEWD |
| 30508 | NAISVGKWD |
| 30509 | NALSVGEWD |
| 30510 | NARSSPDCL |
| 30511 | NCSRHFCEV |
| 30512 | NCWAIHGSW |
| 30513 | NCWAILSSW |
| 30514 | NDLGDDAHG |
| 30515 | NDVADDAHG |
| 30516 | NDVGDDDHG |
| 30517 | NDVGDEAHG |
| 30518 | NDVGGDAHG |
| 30519 | NDVGNDAHG |
| 30520 | NEIDGWQMA |
| 30521 | NEIMMWRAN |
| 30522 | NETMMWRTN |
| 30523 | NFLDYHDMV |
| 30524 | NFMDYNDMV |
| 30525 | NFMEYHDMV |
| 30526 | NHQADEHDA |
| 30527 | NIRPSDASG |
| 30528 | NIRPSDASS |
| 30529 | NIRPSDTSA |
| 30530 | NIRQSDASA |
| 30531 | NIRRSDASA |
| 30532 | NKRNLYAMQ |
| 30533 | NKRNLYAVQ |
| 30534 | NMGIASPWH |
| 30535 | NMVDENRFW |
| 30536 | NMVHEHRFW |
| 30537 | NMVHESRFW |
| 30538 | NMVHQNRFW |
| 30539 | NMVRENRFW |
| 30540 | NSISVGEWD |
| 30541 | NTSCWKLEN |
| 30542 | NTVIRYRHS |
| 30543 | NVNEDVQWQ |
| 30544 | NWVNGFVDH |
| 30545 | NWWAIHSSW |
| 30546 | NYVLFVDHE |
| 30547 | PAWDLVEML |
| 30548 | PAWDLVGMF |
| 30549 | PAWDLVGMH |
| 30550 | PAWDLVGMI |
| 30551 | PAWDPVGML |
| 30552 | PAWELVGMV |
| 30553 | PAWHLVGML |
| 30554 | PCDFDNQPH |
| 30555 | PCHFDNQPH |
| 30556 | PCLDHHRWL |
| 30557 | PCNFDSQPH |
| 30558 | PCNVDNQPH |
| 30559 | PCPDHHRLL |
| 30560 | PCPDHHRRL |
| 30561 | PCPDHHRWV |
| 30562 | PCPDHNRWL |
| 30563 | PCPDNHRWL |
| 30564 | PCPDPHRWL |
| 30565 | PCPEHHRWL |
| 30566 | PCPNVWWHM |
| 30567 | PCQCKVIWQ |
| 30568 | PCTDHHRWL |
| 30569 | PCTFDNQPH |
| 30570 | PCYRKVVFR |
| 30571 | PDGERCERF |
| 30572 | PDWRKIKKF |
| 30573 | PFMKDGECE |
| 30574 | PFMKDVKCE |
| 30575 | PGDPQEDTV |
| 30576 | PGIDLWHMA |
| 30577 | PGIEWWHMA |
| 30578 | PGWIGACMA |
| 30579 | PHFDKCQHF |
| 30580 | PIDWEDVWE |
| 30581 | PKAMAKICE |
| 30582 | PKGMAKICA |
| 30583 | PKGMAKIFE |
| 30584 | PKGMAKIWE |
| 30585 | PKGMARICE |
| 30586 | PKGMSKICE |
| 30587 | PKGMVKICE |
| 30588 | PKGNRTNQI |
| 30589 | PKVMAKICE |
| 30590 | PMHQRWTND |
| 30591 | PMMKKLCCG |
| 30592 | PNGEKCERF |
| 30593 | PQHISADPT |
| 30594 | PQHISSDPT |
| 30595 | PQLISPDPT |
| 30596 | PQRISPDPT |
| 30597 | PRDDESNLI |
| 30598 | PRDKIWFMN |
| 30599 | PRDNVSNLI |
| 30600 | PRFCISLKC |
| 30601 | PRGNESNLI |
| 30602 | PRMSYEIKG |
| 30603 | PRTSYEFKG |
| 30604 | PSKPFSKEE |
| 30605 | PSQPFSEEE |
| 30606 | PTWDLVGML |
| 30607 | PWCECHRLF |
| 30608 | PWSWCEFNK |
| 30609 | PWSWCEYNQ |
| 30610 | PWWTRCKQQ |
| 30611 | PYGEKCERF |
| 30612 | PYTHWKLNN |
| 30613 | QARIEDMAK |
| 30614 | QCEWQKMEH |
| 30615 | QCFLCGSAT |
| 30616 | QCHFLRNCK |
| 30617 | QCMPGCNDL |
| 30618 | QCPVECDHV |
| 30619 | QCVSMDFSG |
| 30620 | QCYREVVFR |
| 30621 | QFAMDWVSD |
| 30622 | QIRIEDMAK |
| 30623 | QKGNRTDQI |
| 30624 | QKGNRTTQI |
| 30625 | QKRGIEAVD |
| 30626 | QKRIEDMAK |
| 30627 | QLNICQATF |
| 30628 | QMHNTFCGW |
| 30629 | QMHSTFCSW |
| 30630 | QRDKVWFMN |
| 30631 | QRDRIWFMN |
| 30632 | QSVDVGLMH |
| 30633 | QTGDWWQYG |
| 30634 | QTGNWWEYG |
| 30635 | QTGNWWKYG |
| 30636 | QTGNWWQYA |
| 30637 | QTGTWWQYG |
| 30638 | QTISGFKDK |
| 30639 | QTISGFQYK |
| 30640 | QTIVAWHHS |
| 30641 | QTLSGFQDK |
| 30642 | QTQIEDMAK |
| 30643 | QTRIDDMAK |
| 30644 | QTRIEDMTK |
| 30645 | QTRIQDMAK |
| 30646 | QTRRLEPYH |
| 30647 | QTRVEDMAK |
| 30648 | QVCIHADTD |
| 30649 | RADVDMTSI |
| 30650 | RCEIWLASS |
| 30651 | RCISNEGMQ |
| 30652 | RCISNEWME |
| 30653 | RCISNEWMP |
| 30654 | RDHNRWNRL |
| 30655 | REEIARPWY |
| 30656 | REKIARPWY |
| 30657 | REQIACPWY |
| 30658 | REQIARPGY |
| 30659 | REQISRPWY |
| 30660 | REQLARPWY |
| 30661 | RETLEQVAY |
| 30662 | RFNLPNFYD |
| 30663 | RFNWPIFYD |
| 30664 | RFNWPNVYD |
| 30665 | RFPFRAQVW |
| 30666 | RFRFLAQVW |
| 30667 | RFRFRAKVW |
| 30668 | RFRFRAQGW |
| 30669 | RFRFRAQIW |
| 30670 | RFRFRTQVW |
| 30671 | RFRFRVQVW |
| 30672 | RFRIRAQVW |
| 30673 | RFRVRAQVW |
| 30674 | RFTWPNFYD |
| 30675 | RHDWQHRTV |
| 30676 | RHELTMSHH |
| 30677 | RHELTMSQY |
| 30678 | RHQPLHYFH |
| 30679 | RHVPLHYFH |
| 30680 | RIDFNVDCY |
| 30681 | RIDINVDCH |
| 30682 | RIDINVDWY |
| 30683 | RIDINVNCY |
| 30684 | RIDINVYCY |
| 30685 | RIDISVDCY |
| 30686 | RIDLNVDCY |
| 30687 | RIDVNVDCY |
| 30688 | RIGINVDCY |
| 30689 | RIMFFDKFT |
| 30690 | RIMYFDEFT |
| 30691 | RIMYFDKCT |
| 30692 | RIMYFDRFT |
| 30693 | RINWPNFYD |
| 30694 | RITYFDKFT |
| 30695 | RKDTPKAGH |
| 30696 | RLARDNNES |
| 30697 | RLARENNEA |
| 30698 | RLELDNNEA |
| 30699 | RLEQYNNEA |
| 30700 | RLERANNEA |
| 30701 | RLERDDNEA |
| 30702 | RLERDHNEA |
| 30703 | RLERDINEA |
| 30704 | RLERDNIEA |
| 30705 | RLERDNNAA |
| 30706 | RLERDNNAV |
| 30707 | RLERDNNED |
| 30708 | RLERDNNEP |
| 30709 | RLERDNNES |
| 30710 | RLERDNNET |
| 30711 | RLERDNTEA |
| 30712 | RLERDTNEA |
| 30713 | RLERHNNEA |
| 30714 | RLERNNNEA |
| 30715 | RLGRDNNEA |
| 30716 | RLKRDNNEA |
| 30717 | RLKRVNNEA |
| 30718 | RLORDNNEA |
| 30719 | RLYSDYWFD |
| 30720 | RMGYEVDWQ |
| 30721 | RMMYFDKFT |
| 30722 | RNLDYNTAV |
| 30723 | RNLNYNTGV |
| 30724 | RNPNYNTAV |
| 30725 | RNVNYNTAV |
| 30726 | RPERDNNEA |
| 30727 | RQQIARPWY |
| 30728 | RRCCISLKC |
| 30729 | RRDSDSVCK |
| 30730 | RRFCVSLKC |
| 30731 | RTECVPDTK |
| 30732 | RTLDSEYFF |
| 30733 | RTLDSEYWF |
| 30734 | RTQCAPDTK |
| 30735 | RTQCVPATK |
| 30736 | RTQCVPDAK |
| 30737 | RTQWVPDTK |
| 30738 | RTVDSEYLF |
| 30739 | RVCPFCCCF |
| 30740 | RVCTFCGCF |
| 30741 | RVDINVDCY |
| 30742 | RVDNDMADG |
| 30743 | RVERDNNEA |
| 30744 | RVKRDNNEA |
| 30745 | RWEKMAYSW |
| 30746 | RYCDAWPDV |
| 30747 | SAWDLVGML |
| 30748 | SCENWGRQH |
| 30749 | SCETGGRQH |
| 30750 | SCETWGRHH |
| 30751 | SCETWGRPH |
| 30752 | SCETWSRQH |
| 30753 | SCETWVRQH |
| 30754 | SCEWSKQCK |
| 30755 | SCMDGTTGV |
| 30756 | SCMHGTTGV |
| 30757 | SCMNGATGV |
| 30758 | SCMNGPTGV |
| 30759 | SCMNGTAGV |
| 30760 | SCMNGTTVV |
| 30761 | SCMNVTTGV |
| 30762 | SCPWPTGEE |
| 30763 | SCQTWGRQH |
| 30764 | SCRHWRPTP |
| 30765 | SCVTWGRQH |
| 30766 | SCWDTQQSI |
| 30767 | SDKWYRVSF |
| 30768 | SFQHAQWMG |
| 30769 | SGWIAACMA |
| 30770 | SGWIRACMA |
| 30771 | SGWNGACMA |
| 30772 | SHFESHQWC |
| 30773 | SHGESHQWC |
| 30774 | SIMMPDYSC |
| 30775 | SITDANLGF |
| 30776 | SKIYAVDQS |
| 30777 | SKRDIENCH |
| 30778 | SKRDVQSMM |
| 30779 | SKREIENCQ |
| 30780 | SKREIETCH |
| 30781 | SKRELENCH |
| 30782 | SKRKIENCH |
| 30783 | SKWACHPKM |
| 30784 | SKWNCHPKM |
| 30785 | SLNELKRDQ |
| 30786 | SMAKHFTSS |
| 30787 | SMHKIQYRA |
| 30788 | SQEFVCAQE |
| 30789 | SRDNESNLI |
| 30790 | SREQVNCDA |
| 30791 | SRISFVCSD |
| 30792 | SRKQVNCDA |
| 30793 | SRLQVNCDA |
| 30794 | SRLSFVCSD |
| 30795 | SRQPVNCDA |
| 30796 | SRQQANCDA |
| 30797 | SRQQVHCDA |
| 30798 | SRQQVNCDV |
| 30799 | SRQQVNCEA |
| 30800 | SRQQVNGDA |
| 30801 | SRQRVNCDA |
| 30802 | SRSMSSSME |
| 30803 | SRSMYSGME |
| 30804 | SRVAFVCSD |
| 30805 | SSAIQCDAA |
| 30806 | SSGIECDAA |
| 30807 | SSGIQCDAT |
| 30808 | SSGIQCEAA |
| 30809 | SSGLQCDAA |
| 30810 | SSILTWTWI |
| 30811 | SSRIQCDAA |
| 30812 | STDNHWGHG |
| 30813 | STQATVRWP |
| 30814 | STQEDFDMF |
| 30815 | STQTSVRWP |
| 30816 | SVQWYRVSF |
| 30817 | SWCDCHRLF |
| 30818 | SWCECHRLL |
| 30819 | SWCECHRLV |
| 30820 | SWCECHRMF |
| 30821 | SWCECHRSF |
| 30822 | SWCECHRVF |
| 30823 | SWCGCHRLF |
| 30824 | SWCVCHRLF |
| 30825 | SWNELKRNQ |
| 30826 | SWSWCEFNQ |
| 30827 | SYTHLKLNN |
| 30828 | SYTPWKLNN |
| 30829 | TAWDLVGML |
| 30830 | TAWRKIKKF |
| 30831 | TCKWVGREN |
| 30832 | TCMFRVYDN |
| 30833 | TCPNVWWHK |
| 30834 | TCPNVWWHL |
| 30835 | TCPNVWWRM |
| 30836 | TCQWFGREN |
| 30837 | TDWPKIKKF |
| 30838 | TDWRKFKKF |
| 30839 | TDWRKLKKF |
| 30840 | TDWRKMKKF |
| 30841 | TDWRKVKKF |
| 30842 | TDWRRIKKF |
| 30843 | TEVKGWESD |
| 30844 | TFMDYHDMV |
| 30845 | TGFHNTFKD |
| 30846 | THQGDEHDA |
| 30847 | TISDIPGEP |
| 30848 | TISDIPGKP |
| 30849 | TLDECCRRG |
| 30850 | TLPCYQRYT |
| 30851 | TMPCYQRYT |
| 30852 | TNVEYFDAW |
| 30853 | TNWREMNAR |
| 30854 | TNWRKIKKF |
| 30855 | TPRPPRSGR |
| 30856 | TPRPSRSAR |
| 30857 | TPRTPRSAR |
| 30858 | TRSGAQAMM |
| 30859 | TSGTCMPIK |
| 30860 | TWMVVHRNN |
| 30861 | TYQWVGREN |
| 30862 | VAEMHWMVN |
| 30863 | VAIGFHWHA |
| 30864 | VAWAVSYVC |
| 30865 | VAWGVSYVG |
| 30866 | VCDIDAYSN |
| 30867 | VCEMGPYDG |
| 30868 | VCEMGPYNG |
| 30869 | VCEMGPYRG |
| 30870 | VCEMGQYHG |
| 30871 | VCERGPYHG |
| 30872 | VCQMGPYHG |
| 30873 | VCVAIMKDQ |
| 30874 | VCVAIMKGK |
| 30875 | VCVAIMQDK |
| 30876 | VCVMGPYHG |
| 30877 | VCWDTQQSI |
| 30878 | VCWMAGAQM |
| 30879 | VDKAWWNLI |
| 30880 | VDKVWWNLV |
| 30881 | VDRSYGPSG |
| 30882 | VFGDFDNHS |
| 30883 | VFVACHVNG |
| 30884 | VGDLNFRIQ |
| 30885 | VHLNADTKF |
| 30886 | VHWDAIMRW |
| 30887 | VHWNAIMRG |
| 30888 | VHWNAIMRL |
| 30889 | VHWNALMRW |
| 30890 | VHWNASMRW |
| 30891 | VHWQQCGHQ |
| 30892 | VIDRIWFNW |
| 30893 | VIKGRSQDK |
| 30894 | VIKLRSQDK |
| 30895 | VIKWGSQDK |
| 30896 | VIKWRGQDK |
| 30897 | VIKWRSLDK |
| 30898 | VIKWRSRDK |
| 30899 | VIQWRSQDK |
| 30900 | VIRWRSQDK |
| 30901 | VLPCQRKRV |
| 30902 | VLWNAIMRW |
| 30903 | VMQNVWCSW |
| 30904 | VMSCHSRDH |
| 30905 | VPKNVNRDM |
| 30906 | VPKTANRDM |
| 30907 | VPKTVDRDM |
| 30908 | VPTTVNRDM |
| 30909 | VQDKCTVNL |
| 30910 | VRHWAFCDY |
| 30911 | VSVQMDRAK |
| 30912 | VTGKMDFWQ |
| 30913 | VTKNVWCSW |
| 30914 | VTKWRSQDQ |
| 30915 | VTMIMMDCH |
| 30916 | VTRDLKKEQ |
| 30917 | VVDMCRKGK |
| 30918 | VVRDLPNQY |
| 30919 | VWFGKCVNC |
| 30920 | VWLKSLDRM |
| 30921 | VWPKGMREM |
| 30922 | VWSCVHTDV |
| 30923 | WDVVAADGP |
| 30924 | WDVVATDVP |
| 30925 | WERAWLFGG |
| 30926 | WERFWHEYW |
| 30927 | WHGRLMITF |
| 30928 | WHQENFVFR |
| 30929 | WKDPFNRKI |
| 30930 | WLANGWFRE |
| 30931 | WLANSWFRG |
| 30932 | WLDNSWFRE |
| 30933 | WLPNSWFRE |
| 30934 | WLSNSWFRE |
| 30935 | WMCRNHHFD |
| 30936 | WMCRNHPFN |
| 30937 | WMCRNHPVD |
| 30938 | WMCRSHPFD |
| 30939 | WRCLCARID |
| 30940 | WRCMCARIA |
| 30941 | WRCMCARLD |
| 30942 | WRCMCARMD |
| 30943 | WRCMCARTD |
| 30944 | WRCMCARVD |
| 30945 | WRCMCSRID |
| 30946 | WRCMCTRID |
| 30947 | WRCWYAPLT |
| 30948 | WRELSNTGY |
| 30949 | WREWSHTGY |
| 30950 | WREWSNAGY |
| 30951 | WREWSNTAY |
| 30952 | WREWSNTCY |
| 30953 | WREWSNTVY |
| 30954 | WRGWSNTGY |
| 30955 | WRQWSNTGY |
| 30956 | WRRFASGWE |
| 30957 | WRRFASWLE |
| 30958 | WRRFSSWWE |
| 30959 | WRRFVSWWE |
| 30960 | WRTADEGDA |
| 30961 | WRTADESDT |
| 30962 | WRTAHESDA |
| 30963 | WRTGDESDA |
| 30964 | WTETTVRWP |
| 30965 | WTQTTVRWP |
| 30966 | WVANSWFRE |
| 30967 | WVCRNHPFD |
| 30968 | WYGHLNAMC |
| 30969 | WYSWRVRNR |
| 30970 | YARGIWDIH |
| 30971 | YARGIWDVH |
| 30972 | YARGTWDFH |
| 30973 | YCSRHFCEV |
| 30974 | YDRGIWDFH |
| 30975 | YEVHSFLSG |
| 30976 | YHDLCFAWY |
| 30977 | YHDLFFDWY |
| 30978 | YHDLFFGWY |
| 30979 | YKREIENCH |
| 30980 | YMGIASPWH |
| 30981 | YMWWEEGTL |
| 30982 | YMWWQAGTL |
| 30983 | YRQQVNCDA |
| 30984 | YRRLQSNTT |
| 30985 | YRRWESNTT |
| 30986 | YRRWQSNTI |
| 30987 | YRRWQSNTS |
| 30988 | YVKFNVNIN |
| 30989 | YVKFSVDIN |
| 30990 | YVKISVNIN |

### Example 40

AAV5 Variants with Skin Tissue Tropism as Identified by Machine Learning

This example describes preferred properties of engineered AAV5 variants that display skin tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display skin tissue tropism.

Favored biophysical properties and favored amino acid residues at each position in the 581 to 589 region of an AAV5 VP capsid polypeptide, which are associated with a higher probability of skin tissue tropism are described below. Any of the below machine learning-derived positional amino acid preferences described in TABLE 64 can be present alone or in combination with each other or in combination with any of the amino acid preferences observed and described in EXAMPLE 17, TABLE 33. Listed below in TABLE 64 are a summary of positional features shared between the top important features for skin tropism extracted from the ML models.

TABLE 64
Machine Learning-Derived Skin Tissue Tropism Rules

Low surface accessibility at Xaa1
Xaa1 is selected from C
Low volume at Xaa1
Xaa1 is selected from C
Low mutability at Xaa1
Xaa1 is selected from C
High surface accessibility at Xaa2
Xaa2 is selected from R or K
High average flexibility at Xaa2
Xaa2 is selected from K, I, or N
Low mutability at Xaa2
Xaa2 is selected from P or K
High hydropathy at Xaa3
Xaa3 is selected from I or V
Low mutability at Xaa4
Xaa4 is selected from L, F, or Y
Low average flexibility at Xaa4
Xaa4 is selected from W, H, F, or M
High average flexibility at Xaa5
Xaa5 is selected from G, R, K, I, or N
High average flexibility at Xaa6
Xaa6 is selected from G, R, K, I, or N
High surface accessibility at Xaa8
Xaa8 is selected from M, G, or F
Low average flexibility at Xaa8
Xaa8 is selected from H, F, M, or W
Low mutability at Xaa8
Xaa8 is selected from L, F, or Y
High average flexibility at Xaa9
Xaa9 is selected from D, E, R, K, P, or G
High mutability at Xaa9
Xaa9 is selected from D, E, R, V, A, or H

TABLE 65 below provides sequences of the 581 to 589 region for variant AAV VP1 capsid polypeptides that exhibited skin tissue tropism and comport to one or more of the rules provided in TABLE 64. The present disclosure, thus, provides for rAAVs composed of engineered AAV5 VP1 capsid polypeptides having a SEQ ID NO: 2, wherein the Xaa1 to Xaa9 region is any one of SEQ ID NO: 32991-SEQ ID NO: 33990, as disclosed in TABLE 65. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 65
Sequences of the 581 to 589 Region in AAV5
VP1 Capsid Polypeptide that Drive Skin
Tissue Tropism

| SEQ | 581-589 |
|---|---|
| ID NO | Sequence |
| 32991 | AADAETPWF |
| 32992 | AADPRADCP |
| 32993 | AADPRGDCP |
| 32994 | AADPRIDCP |
| 32995 | AADPRVDCA |
| 32996 | AADPRVDCL |
| 32997 | AADPRVDCR |
| 32998 | AADPRVECP |
| 32999 | AADPTKSWC |
| 33000 | AADSETPWY |
| 33001 | AASPYITYR |
| 33002 | AEHCYRHKH |
| 33003 | AEPFPCDWS |
| 33004 | AFFYMDKLS |
| 33005 | AIVCRVNYP |
| 33006 | AKRDMDPAQ |
| 33007 | AKRDMDPAS |
| 33008 | AKRNMDPAP |
| 33009 | ANETNDNFH |
| 33010 | ANVGCIMIL |
| 33011 | ASARYIDFT |
| 33012 | ASWCSDERC |
| 33013 | ATAGCIMIL |
| 33014 | ATDPRVDCP |
| 33015 | ATVGFIMIL |
| 33016 | AVATRAMYY |
| 33017 | AVDPRVDCP |
| 33018 | CAFQCYRSP |
| 33019 | CAFVQQNFQ |
| 33020 | CAGMQATGD |
| 33021 | CAHHLNOTI |
| 33022 | CAIFNTVDQ |
| 33023 | CAIHTQSHS |
| 33024 | CAIMCSAGP |
| 33025 | CAIMFSAGQ |
| 33026 | CAIMFSAGT |
| 33027 | CAIMLKHMT |
| 33028 | CAIMLQHMA |
| 33029 | CAIMLQHMI |
| 33030 | CALHTRSHS |
| 33031 | CALMFSAGP |
| 33032 | CALPVRVAM |
| 33033 | CALPYAGSR |
| 33034 | CALSMYSHE |
| 33035 | CANHLNKTI |
| 33036 | CANHLNQTM |
| 33037 | CANHLNRTI |
| 33038 | CANHLSQTI |
| 33039 | CANHVNQTI |
| 33040 | CAPISAAPV |
| 33041 | CARLIDWCC |
| 33042 | CASMFSAGP |
| 33043 | CATHLNQTI |
| 33044 | CATVKMWHT |
| 33045 | CAVMFSAGP |
| 33046 | CAVMKFSEH |
| 33047 | CAVYFAKCA |
| 33048 | CAVYFGKCV |
| 33049 | CAVYFGRCA |
| 33050 | CAVYYGKCA |
| 33051 | CAYFKRHPD |
| 33052 | CAYFSMMDR |
| 33053 | CAYHLNQTI |
| 33054 | CCLYYVNVE |
| 33055 | CCTIKQMDI |
| 33056 | CCVWEFNYP |
| 33057 | CDHQFIDQA |
| 33058 | CEHFKRHPD |
| 33059 | CERLTQNVD |
| 33060 | CEYFKRDPD |
| 33061 | CEYFKRHPA |
| 33062 | CEYFKRHPG |
| 33063 | CEYFKRHTD |
| 33064 | CEYFKRRPD |
| 33065 | CEYFKRYPD |
| 33066 | CEYIKRHPD |
| 33067 | CFHPYHQAH |
| 33068 | CFHQYNQAH |
| 33069 | CFLWEFNYP |
| 33070 | CFMALSFHW |
| 33071 | CFMGLSFHL |
| 33072 | CFMVLSFHW |
| 33073 | CFPVYKDWS |
| 33074 | CFVGLSFHW |
| 33075 | CFVLEFNYP |
| 33076 | CFVWAFNYP |
| 33077 | CFVWEFDYP |
| 33078 | CFVWEFNDP |
| 33079 | CFVWEFNHP |
| 33080 | CFVWEFNSP |
| 33081 | CFVWEFSYP |
| 33082 | CFVWGFNYP |
| 33083 | CFVWGINYP |
| 33084 | CFVWQFNYP |
| 33085 | CFVWVFNYP |
| 33086 | CFYQYHQAH |
| 33087 | CHAPSIMGP |
| 33088 | CHCHIRHTG |
| 33089 | CHFMFNAGR |
| 33090 | CHFMIHAGR |
| 33091 | CHFMINAAR |
| 33092 | CHFMINACR |
| 33093 | CHFMINAGH |
| 33094 | CHFMINDGR |
| 33095 | CHFMINSGR |
| 33096 | CHFMINTGR |
| 33097 | CHFMINVGR |
| 33098 | CHFMLNAGR |
| 33099 | CHFMSNAGR |
| 33100 | CHFMTNAGR |
| 33101 | CHFMVNAGR |
| 33102 | CHFVINAGR |
| 33103 | CHHATNTSQ |
| 33104 | CHHFATASE |
| 33105 | CHHFATETE |
| 33106 | CHHFATQSE |
| 33107 | CHIMINAGR |
| 33108 | CHPVYKDWS |
| 33109 | CHSMINAGR |
| 33110 | CHTIKQMDI |
| 33111 | CHVMINAGR |
| 33112 | CHYMINAGR |
| 33113 | CIKACNDHE |
| 33114 | CIKACYDRE |
| 33115 | CIKACYDYE |
| 33116 | CIKACYHHE |
| 33117 | CIKACYNHE |
| 33118 | CIKAYNVMM |
| 33119 | CIKAYYDHE |
| 33120 | CIKGCYDHE |
| 33121 | CIKPCYDHE |
| 33122 | CIKSCYDHE |
| 33123 | CIKSYNVMV |
| 33124 | CIKTYNVMM |
| 33125 | CIKYYNVMM |
| 33126 | CILHKHNFP |
| 33127 | CILHKHSSP |
| 33128 | CILHKHSVP |
| 33129 | CILHKNSFP |
| 33130 | CILQKHSFP |
| 33131 | CILYKHSFP |
| 33132 | CIMHKHSFP |
| 33133 | CIQACYDHE |
| 33134 | CIQFTKHQE |
| 33135 | CIQLSKMGP |
| 33136 | CIQLTKMGP |
| 33137 | CIRSYNVMM |
| 33138 | CITACYDHE |
| 33139 | CITMVRSQT |
| 33140 | CKAHCPGQY |
| 33141 | CKCHFDQVE |
| 33142 | CKDFHWMND |
| 33143 | CKFDITKMA |
| 33144 | CKFDMASFF |
| 33145 | CKFDMPCFF |
| 33146 | CKFDMPGFF |
| 33147 | CKFDMPSCF |
| 33148 | CKFDMPSFI |
| 33149 | CKFDMPSFV |
| 33150 | CKFDMPSFY |
| 33151 | CKFDMPSVF |
| 33152 | CKFDMPTFF |
| 33153 | CKFDMQGFF |
| 33154 | CKFDMQSFF |
| 33155 | CKFDMSSFF |
| 33156 | CKFDMTSFF |
| 33157 | CKFHMPSFF |
| 33158 | CKFNMPSFF |
| 33159 | CKFYIPSFF |
| 33160 | CKGHFDQVE |
| 33161 | CKHFHWMND |
| 33162 | CKIDMPSFF |
| 33163 | CKIHFDQVE |
| 33164 | CKNFHLMND |
| 33165 | CKNFHRMND |
| 33166 | CKNFHWMDD |
| 33167 | CKNFHWMHD |
| 33168 | CKNFHWMNE |
| 33169 | CKNFHWMNH |
| 33170 | CKNFHWMSD |
| 33171 | CKNFHWRND |
| 33172 | CKNFLWMND |
| 33173 | CKNFPWMND |
| 33174 | CKNFYWMND |
| 33175 | CKNHFDQVE |
| 33176 | CKNIHWMND |
| 33177 | CKPQINDAY |
| 33178 | CKQQINDAC |
| 33179 | CKQQINDAD |
| 33180 | CKQQINDAF |
| 33181 | CKQQINDAH |
| 33182 | CKQQINDAN |
| 33183 | CKQQINDAS |
| 33184 | CKQQINDDY |
| 33185 | CKQQINDGY |
| 33186 | CKQQINDSY |
| 33187 | CKQQINHAY |
| 33188 | CKQQLNDAY |
| 33189 | CKRQINDAY |
| 33190 | CKSFHWMND |
| 33191 | CKSHFAQVE |
| 33192 | CKSHFDKVE |
| 33193 | CKSHFNQVE |
| 33194 | CKSHIDQVE |
| 33195 | CKSHVDQVE |
| 33196 | CKTFHWMND |
| 33197 | CKTHFDQVE |
| 33198 | CKTQCPGQY |
| 33199 | CKVDMPSFF |
| 33200 | CKYDFTKMA |
| 33201 | CKYDIPKMA |
| 33202 | CKYDISKMA |
| 33203 | CKYDITKMG |
| 33204 | CKYDITKMS |
| 33205 | CKYDITKMT |
| 33206 | CKYDITKMV |
| 33207 | CKYDITMMA |
| 33208 | CKYDITQMA |
| 33209 | CKYDLTKMA |
| 33210 | CKYDMPSFF |
| 33211 | CKYDNTKMA |
| 33212 | CKYDTTKMA |
| 33213 | CKYDVTKMA |
| 33214 | CKYFHWMND |
| 33215 | CLELRTQYA |
| 33216 | CLELVKAYE |
| 33217 | CLELVKDYE |
| 33218 | CMFHNHKGF |
| 33219 | CMFQCYRSP |
| 33220 | CMFVQQNFQ |
| 33221 | CMHLSCDWA |
| 33222 | CMHLVNMNS |
| 33223 | CMKACYDHE |
| 33224 | CMKFIRVGS |
| 33225 | CMPFIRVGS |
| 33226 | CMQFIRAGS |
| 33227 | CMQFIRIGS |
| 33228 | CMQFIRLGS |
| 33229 | CMQFIRVAS |
| 33230 | CMQFIRVGG |
| 33231 | CMQFIRVGN |
| 33232 | CMQFLRVGS |
| 33233 | CMQFNRVGS |
| 33234 | CMQFSRVGS |
| 33235 | CMQFTRVGS |
| 33236 | CMQFVRVGS |
| 33237 | CMQVIRVGS |
| 33238 | CMQYIRVGS |
| 33239 | CMRFIRVGS |
| 33240 | CMSWLTNQE |
| 33241 | CMVHNHKGS |
| 33242 | CMVHNHQGF |
| 33243 | CMYWLNNQE |
| 33244 | CMYWLTNKE |
| 33245 | CNCNLPYAI |
| 33246 | CNCPIRHTG |
| 33247 | CNCQIRHTG |
| 33248 | CNFMINAGR |
| 33249 | CNFQTNVHM |
| 33250 | CNFQTSVHL |
| 33251 | CNHKFIDQA |
| 33252 | CNHKVNSWI |
| 33253 | CNHPFIDQA |
| 33254 | CNHQFIDQG |
| 33255 | CNHQFIDQS |
| 33256 | CNHQFIDQT |
| 33257 | CNHQFIDQV |
| 33258 | CNHQFLDQA |
| 33259 | CNPQFIDQA |
| 33260 | CNYQTNVHL |
| 33261 | CPLPFLIKE |
| 33262 | CPKPVLIQE |
| 33263 | CQELRTQHA |
| 33264 | CQELRTQYT |
| 33265 | CQEVHAHMS |
| 33266 | CQEVYAHMT |
| 33267 | CQKWRSIWK |
| 33268 | CQQVYAHMS |
| 33269 | CQSHFDQVE |
| 33270 | CRAAHPVCA |
| 33271 | CRAAHSICA |
| 33272 | CRAAYSVCA |
| 33273 | CRATHSVCA |
| 33274 | CRAVHSVCA |
| 33275 | CRFMINAGR |
| 33276 | CRTAHSVCA |
| 33277 | CRVAHSVCA |
| 33278 | CSAMVRSQT |
| 33279 | CSGCHMKMP |
| 33280 | CSGCYMKMS |
| 33281 | CSHPVHDNE |
| 33282 | CSLHKHSFP |
| 33283 | CSLPYAWSR |
| 33284 | CSMIQNSFP |
| 33285 | CSTMVRSQA |
| 33286 | CSVCYMKMP |
| 33287 | CSVWEFNYP |
| 33288 | CTAMLQRKG |
| 33289 | CTAVKMWHT |
| 33290 | CTCGYNRGD |
| 33291 | CTCHIRHTG |
| 33292 | CTCNLPSAI |
| 33293 | CTCNVPYAI |
| 33294 | CTCWYNRGE |
| 33295 | CTEVLQRKG |
| 33296 | CTGCYMKMP |
| 33297 | CTGIQATGD |
| 33298 | CTGLLMHKE |
| 33299 | CTGQLMHEE |
| 33300 | CTGQLMHQE |
| 33301 | CTGQLMRKE |
| 33302 | CTGWYNRGD |
| 33303 | CTHAVHDNE |
| 33304 | CTHGFLTAP |
| 33305 | CTHGFLTSP |
| 33306 | CTHGFLTVP |
| 33307 | CTHGVLTGP |
| 33308 | CTHKVNTWI |
| 33309 | CTHPVHDHE |
| 33310 | CTHPVNDNE |
| 33311 | CTHQFIDQA |
| 33312 | CTHQVHDNE |
| 33313 | CTHTVHDNE |
| 33314 | CTHVFLTGP |
| 33315 | CTIFHTVDQ |
| 33316 | CTIFNNVDQ |
| 33317 | CTIFNTVDK |
| 33318 | CTIFTTVDQ |
| 33319 | CTIMFSAGP |
| 33320 | CTIRANLHF |
| 33321 | CTIYRNLLS |
| 33322 | CTKACYDHE |
| 33323 | CTKMLQRKG |
| 33324 | CTKSYNVMM |
| 33325 | CTLKVNSWI |
| 33326 | CTLMHIHTR |
| 33327 | CTLMHSHAR |
| 33328 | CTLMHSHNR |
| 33329 | CTLMHSHTI |
| 33330 | CTLMLSHTR |
| 33331 | CTMLANHDQ |
| 33332 | CTMLANHHQ |
| 33333 | CTMLANHYK |
| 33334 | CTMLANHYP |
| 33335 | CTMLANHYR |
| 33336 | CTMLANNYQ |
| 33337 | CTMLANYYQ |
| 33338 | CTMMANHYQ |
| 33339 | CTMVANHYQ |
| 33340 | CTMWANHYQ |
| 33341 | CTNFHWMND |
| 33342 | CTNVKMWHT |
| 33343 | CTNYHLKAG |
| 33344 | CTNYHVKAV |
| 33345 | CTPVKMWHT |
| 33346 | CTQFIKHQE |
| 33347 | CTQFPKHOE |
| 33348 | CTQFTQHQE |
| 33349 | CTQFTTHQE |
| 33350 | CTQLNKMGP |
| 33351 | CTTAWQGTW |
| 33352 | CTTFKMWHT |
| 33353 | CTTGWQGAW |
| 33354 | CTTVKMLHT |
| 33355 | CTTVKMWHA |
| 33356 | CTTVKMWHI |
| 33357 | CTTVKMWNT |
| 33358 | CTTVQMWHT |
| 33359 | CTTYRNLMS |
| 33360 | CTTYRNLPS |
| 33361 | CTTYRNVLS |
| 33362 | CTVFNTVDQ |
| 33363 | CTVLANHYQ |
| 33364 | CTYFSMMDP |
| 33365 | CTYFSMTDR |
| 33366 | CTYKVNSWI |
| 33367 | CTYVSMMDR |
| 33368 | CVAMKFSEH |
| 33369 | CVEPYTDHQ |
| 33370 | CVFQCNRSP |
| 33371 | CVFQCYGSP |
| 33372 | CVFQCYRGP |
| 33373 | CVFQCYRNP |
| 33374 | CVFQCYRTP |
| 33375 | CVFQYYRSP |
| 33376 | CVFVPQNFQ |
| 33377 | CVFVQENFQ |
| 33378 | CVFVQQHFQ |
| 33379 | CVFVQQSFQ |
| 33380 | CVFVQQTFQ |
| 33381 | CVFVRONFQ |
| 33382 | CVGMKFSEH |
| 33383 | CVHLVHWQW |
| 33384 | CVHQYHQAH |
| 33385 | CVLMKFSEH |
| 33386 | CVLVQQNFP |
| 33387 | CVMGLSFHW |
| 33388 | CVRMIDWCC |
| 33389 | CVSISAAPV |
| 33390 | CVVMKFNEH |
| 33391 | CVVMKFSED |
| 33392 | CVVMKFSER |
| 33393 | CVVMKFSKH |
| 33394 | CVVMKFSVH |
| 33395 | CVVMKFTEH |
| 33396 | CVVMKYSEH |
| 33397 | CVVMTFSEH |
| 33398 | CVVVQQNFQ |
| 33399 | CVVWEFNYP |
| 33400 | CVYQCYRSP |
| 33401 | CVYWLTNQE |
| 33402 | CWAVTSMGS |
| 33403 | CWMPQQVAA |
| 33404 | CWVVTSMSS |
| 33405 | CYAIKQMDI |
| 33406 | CYHTTNTSQ |
| 33407 | CYHVYKDWS |
| 33408 | CYMGLSFHW |
| 33409 | CYPFYKDWS |
| 33410 | CYPLYKDWS |
| 33411 | CYPVSKDWS |
| 33412 | CYPVYKAWS |
| 33413 | CYPVYKDWT |
| 33414 | CYPVYKNWS |
| 33415 | CYPVYQDWS |
| 33416 | CYPVYTDWS |
| 33417 | CYSVYKDWS |
| 33418 | CYTIKHMDI |
| 33419 | CYTIKKMDI |
| 33420 | CYTIKQMAI |
| 33421 | CYTIKQMDL |
| 33422 | CYTIKQMDM |
| 33423 | CYTIKQMDS |
| 33424 | CYTIKQMGI |
| 33425 | CYTIKQMHI |
| 33426 | CYTIKQMNI |
| 33427 | CYTIKQVDI |
| 33428 | CYTMKQMDI |
| 33429 | CYTVYKDWS |
| 33430 | DEPCPCDWS |
| 33431 | DEPFPCDWA |
| 33432 | DEPFPCDWT |
| 33433 | DESFPCDWS |
| 33434 | DFVRTNLGV |
| 33435 | DHANQRVDR |
| 33436 | DIFERSMMF |
| 33437 | DKAGYGCHE |
| 33438 | DKEDPQQWL |
| 33439 | DKEEPEQWL |
| 33440 | DKGGNGCHE |
| 33441 | DKGGYGCNE |
| 33442 | DKGGYGGHE |
| 33443 | DKGGYSCHE |
| 33444 | DKSEESRQE |
| 33445 | DKSPWGANH |
| 33446 | DKVGYGCHE |
| 33447 | DQDGRCYKG |
| 33448 | DQDGRCYKQ |
| 33449 | DSEPSRGDH |
| 33450 | DSQPSRGDY |
| 33451 | DYAHQRVDR |
| 33452 | DYANERVDR |
| 33453 | DYANKRVDR |
| 33454 | DYANQRGDR |
| 33455 | DYATQRVDR |
| 33456 | DYCRRTFHH |
| 33457 | DYQPCHVHM |
| 33458 | DYSNQRVDR |
| 33459 | DYTNQRVDR |
| 33460 | EDESRHISW |
| 33461 | EDEYRHMSW |
| 33462 | EDKSRHMSW |
| 33463 | EFVTRKAAD |
| 33464 | EFVTRKAAT |
| 33465 | EGEDRQAFW |
| 33466 | EHCNHDQGG |
| 33467 | EIFERSMMY |
| 33468 | EKAPWGANH |
| 33469 | ESARYIDFA |
| 33470 | ESARYLDFT |
| 33471 | FAEPYTDHQ |
| 33472 | FAERTTDHR |
| 33473 | FAQRATDHR |
| 33474 | FAQRNTDHR |
| 33475 | FAQRTTHHR |
| 33476 | FAQRTTNHR |
| 33477 | FFGTPYHMS |
| 33478 | FGHWAQDAS |
| 33479 | FGQLIIKHR |
| 33480 | FGQRTTDHR |
| 33481 | FHDRHFTFT |
| 33482 | FHDRPFNFT |
| 33483 | FHDRPFTFA |
| 33484 | FICLIKSAS |
| 33485 | FICQIKSAA |
| 33486 | FKCAHDPTG |
| 33487 | FKMQMQYDS |
| 33488 | FKVQMQYDN |
| 33489 | FRAGMIVSC |
| 33490 | FRDAMIVSC |
| 33491 | FRDGMIFSC |
| 33492 | FRDGMTVSC |
| 33493 | FRDRPFTFT |
| 33494 | FRHGMIVSC |
| 33495 | FRNGMIVSC |
| 33496 | FTQQRCAQC |
| 33497 | FTQQRCGQC |
| 33498 | FTQRTTDHR |
| 33499 | FTRQRCVQC |
| 33500 | FVEPYTDHR |
| 33501 | FVEPYTHHQ |
| 33502 | FVQPYTDHQ |
| 33503 | FYWCCNREC |
| 33504 | FYWCYTREC |
| 33505 | FYWFCTREC |
| 33506 | GAAVIRKMQ |
| 33507 | GFAVIRKMQ |
| 33508 | GGWIMRSKR |
| 33509 | GIAVIRKMQ |
| 33510 | GILPSVKFQ |
| 33511 | GILPYGKFQ |
| 33512 | GILPYVKVQ |
| 33513 | GKCGFDVAA |
| 33514 | GKFDMPSFF |
| 33515 | GLAVIRKMQ |
| 33516 | GLEHCRPGT |
| 33517 | GLEWHTKNW |
| 33518 | GLQWHTKDW |
| 33519 | GNETSDNFH |
| 33520 | GSQWLNDSF |
| 33521 | GVAAIRKMQ |
| 33522 | GVAGIRKMQ |
| 33523 | GVAIIRKMQ |
| 33524 | GVALIRKMQ |
| 33525 | GVAVFRKMQ |
| 33526 | GVAVIRKME |
| 33527 | GVAVIRKMR |
| 33528 | GVEWHTKDW |
| 33529 | GVFPNMFAH |
| 33530 | GVFPNMFSP |
| 33531 | GVTVIRKMQ |
| 33532 | GVVVIRKMQ |
| 33533 | GVWIVRSKR |
| 33534 | GYANQRVDR |
| 33535 | HCCFKNGDL |
| 33536 | HCCFKNVDM |
| 33537 | HCCFKSGDM |
| 33538 | HCCFKTGDM |
| 33539 | HCCFKYGDM |
| 33540 | HCCFRNGDM |
| 33541 | HKVNLKGDE |
| 33542 | HKVTIFKDR |
| 33543 | HQLEFDFVG |
| 33544 | HQMEFDFVG |
| 33545 | HRVTIFKDR |
| 33546 | HYCRSGDVQ |
| 33547 | IAAQIDDRE |
| 33548 | IAAQSDDRQ |
| 33549 | IAARSDDRE |
| 33550 | IADQSDDRE |
| 33551 | IAVCVIRDN |
| 33552 | ICARWHCGP |
| 33553 | ICLLITKTE |
| 33554 | ICLRWHCGP |
| 33555 | ICVRWHCGS |
| 33556 | ICVRWHCGT |
| 33557 | ICWLFTKTE |
| 33558 | ICWLIAKTE |
| 33559 | ICWLIKKTE |
| 33560 | ICWLIPKTE |
| 33561 | ICWLISKTE |
| 33562 | ICWLITKNE |
| 33563 | ICWLITKTG |
| 33564 | ICWLITKTQ |
| 33565 | ICWSITKTE |
| 33566 | ICWWITKTE |
| 33567 | ISACVIRDN |
| 33568 | ISCRTQSDC |
| 33569 | ISVCVIRDT |
| 33570 | IVQQIRDFH |
| 33571 | IWECPRAGS |
| 33572 | IWECPRLGS |
| 33573 | KADRFMVDQ |
| 33574 | KAEHEEMWN |
| 33575 | KAEYFDASA |
| 33576 | KCAFNGIDE |
| 33577 | KCDYRNMHF |
| 33578 | KELLCSDDA |
| 33579 | KGDRFMVDP |
| 33580 | KGKPWGHHN |
| 33581 | KHCHSMQFG |
| 33582 | KHCRSMQFE |
| 33583 | KHCYSMQFE |
| 33584 | KIVCRVNYP |
| 33585 | KKLNIQKQS |
| 33586 | KLCFKEDAF |
| 33587 | KMCSSAKSY |
| 33588 | KMCTLTKAD |
| 33589 | KMFSSAKSY |
| 33590 | KMYPSAKSY |
| 33591 | KMYSSVKSY |
| 33592 | KQCHSMQFE |
| 33593 | KSAMPMNDD |
| 33594 | KSARCTLHM |
| 33595 | KSCLYFDAC |
| 33596 | KSCLYFDAF |
| 33597 | KSCLYFDSY |
| 33598 | KSSWTDSFG |
| 33599 | KSVRCKLHM |
| 33600 | KVCFKEDAI |
| 33601 | KVCFKEDGF |
| 33602 | KVCFKEDPF |
| 33603 | KVCFKEDSF |
| 33604 | KVCYKEDAF |
| 33605 | KWCLSIDYR |
| 33606 | KWFLSIDYP |
| 33607 | KWFLSIDYS |
| 33608 | KWFLSMDYR |
| 33609 | KWFPSIDYR |
| 33610 | KWFVSIDYR |
| 33611 | KWVLSIDYR |
| 33612 | KYARCKLHM |
| 33613 | LAAQSDDRE |
| 33614 | LAARIGEVK |
| 33615 | LAARIGKVE |
| 33616 | LAARSGEVE |
| 33617 | LCVRWHCGP |
| 33618 | LCWLITKTE |
| 33619 | LGIRNMELR |
| 33620 | LKHPAWKEE |
| 33621 | LKHPIWKEE |
| 33622 | LKHPNWKEE |
| 33623 | LKHPPWKEE |
| 33624 | LKHPSWKEE |
| 33625 | LKHPTWREE |
| 33626 | LKHTTWKEE |
| 33627 | LKLPTWKEE |
| 33628 | LKPPTWKEE |
| 33629 | MAANCEVFG |
| 33630 | MANENRGIR |
| 33631 | MANENRGVH |
| 33632 | MANENRVIH |
| 33633 | MASENRGIH |
| 33634 | MATPERQMP |
| 33635 | MAVNCEVFA |
| 33636 | MCIRSGFVV |
| 33637 | MCIRSGIIV |
| 33638 | MCLRSGFIV |
| 33639 | MCVRSGFIV |
| 33640 | MCWLITKTE |
| 33641 | MEDRLAHYY |
| 33642 | MEDRVAHYH |
| 33643 | MKHPTWKEE |
| 33644 | MKIPNIQTI |
| 33645 | MKMPRWGMP |
| 33646 | MKMTRWGML |
| 33647 | MKMTRWGMQ |
| 33648 | MKMTRWGMS |
| 33649 | MKTPTIQTI |
| 33650 | MKVTRWGMP |
| 33651 | MQNACFKNP |
| 33652 | MSVCVIRDN |
| 33653 | MVAHNRLAN |
| 33654 | MVANCEVFA |
| 33655 | MVEHNRLAD |
| 33656 | MVEHNRLAH |
| 33657 | MVEHNRLDN |
| 33658 | MVEHNRLGN |
| 33659 | MVEHNRVAN |
| 33660 | MVVYREHMG |
| 33661 | MVVYRENMA |
| 33662 | MVVYRETMG |
| 33663 | MYAERNADM |
| 33664 | MYVWRNDFA |
| 33665 | MYVWRNDGA |
| 33666 | NAIRIFLFG |
| 33667 | NCCFKNGDM |
| 33668 | NCIHNWKHA |
| 33669 | NCIHNWQHT |
| 33670 | NFFYMDKLS |
| 33671 | NFQHVRMLE |
| 33672 | NFYETLKRI |
| 33673 | NGIRIFLFA |
| 33674 | NGIRIFLFV |
| 33675 | NGIRNFLFG |
| 33676 | NGIRSFLFG |
| 33677 | NGLRIFLFG |
| 33678 | NGVRIFLFG |
| 33679 | NKCAHDPTG |
| 33680 | NKCGFDVAA |
| 33681 | NSCRRTFHH |
| 33682 | NYANQRVDR |
| 33683 | NYCRRTFHL |
| 33684 | NYCRRTFHP |
| 33685 | NYCRRTFHQ |
| 33686 | NYCRRTFHR |
| 33687 | NYCRRTFHY |
| 33688 | NYCRRTFYH |
| 33689 | PANHARNPE |
| 33690 | PANHARNPQ |
| 33691 | PANHARNPR |
| 33692 | PANHARTPK |
| 33693 | PVCPTRYAD |
| 33694 | PVNHARNPK |
| 33695 | QAESRDVLD |
| 33696 | QAKSRDGLD |
| 33697 | QAKSRDVLA |
| 33698 | QAKSRDVLN |
| 33699 | QAKSRDVMD |
| 33700 | QCAYRNMHF |
| 33701 | QCCFKNGDM |
| 33702 | QCDYRDMHF |
| 33703 | QCDYRNMDF |
| 33704 | QCDYRNMNF |
| 33705 | QCHYRNMHF |
| 33706 | QCNYRNMHF |
| 33707 | QCVYRNMHF |
| 33708 | QFDYRNMHF |
| 33709 | QGGRILTSA |
| 33710 | QGKSRDVLD |
| 33711 | QGNDAIVWQ |
| 33712 | QHCNHDQGV |
| 33713 | QKIPVNDHQ |
| 33714 | QKLDIQKQS |
| 33715 | QKLKIQKQS |
| 33716 | QKLNIKKQS |
| 33717 | QKLNIQKQP |
| 33718 | QKLNMQKQS |
| 33719 | QKLNVQKQS |
| 33720 | QKLPVNDHQ |
| 33721 | QKVHVNDHQ |
| 33722 | QKVNIQKQS |
| 33723 | QKVPLNDHQ |
| 33724 | QKVPVSDHQ |
| 33725 | QKVPVTDHQ |
| 33726 | QKVQVNDHQ |
| 33727 | QMCTLTKTD |
| 33728 | QQVTIFKDR |
| 33729 | QSARYIDFT |
| 33730 | QTKGRDGAC |
| 33731 | QVKSRDVLD |
| 33732 | QWTRWLIVT |
| 33733 | RACFEMKHI |
| 33734 | RADVTNHRY |
| 33735 | RAQSQWDGC |
| 33736 | RASCGISMA |
| 33737 | RAVTPCHYN |
| 33738 | RCCFKNGDM |
| 33739 | RCVQPQKEA |
| 33740 | RCVQPQKKG |
| 33741 | RDLMYTDKA |
| 33742 | RHKWRSIWK |
| 33743 | RINPDIIAP |
| 33744 | RISHDIIAP |
| 33745 | RISPDIIAL |
| 33746 | RISPDIIAS |
| 33747 | RISPDIIGP |
| 33748 | RISPDILAP |
| 33749 | RISPDTIAP |
| 33750 | RISPEIIAP |
| 33751 | RISPNIIAP |
| 33752 | RISRDIIAP |
| 33753 | RIVCRVNYP |
| 33754 | RKHGLLFMG |
| 33755 | RKHGLLFMT |
| 33756 | RKHGLLFMV |
| 33757 | RKHGMLFMA |
| 33758 | RKHPTWKEE |
| 33759 | RKLNIQKQS |
| 33760 | RLHGMGDVP |
| 33761 | RMAHNFWDA |
| 33762 | RMAHTFWNA |
| 33763 | RMAPTFWDA |
| 33764 | RMARTFWDA |
| 33765 | RMHVLVIYT |
| 33766 | RMSPDIIAP |
| 33767 | RMYSSAKSY |
| 33768 | RPCFEMKHI |
| 33769 | RQEWRSIWK |
| 33770 | RQKLRSIWK |
| 33771 | RQKWRIIWK |
| 33772 | RQKWRNIWK |
| 33773 | RQKWRSFWK |
| 33774 | RQKWRSIWE |
| 33775 | RQKWRSIWQ |
| 33776 | RQKWRSIWR |
| 33777 | RQKWRSIWT |
| 33778 | RQKWRSMWK |
| 33779 | RQKWRSVWK |
| 33780 | RQQWRSIWK |
| 33781 | RQRWRSIWK |
| 33782 | RQTWRSIWK |
| 33783 | RSATPCHYN |
| 33784 | RSCFEMKHL |
| 33785 | RSCFEMRHI |
| 33786 | RSCFVMKHI |
| 33787 | RSCYEMKHI |
| 33788 | RSMWPPSSV |
| 33789 | RSVTPCHSN |
| 33790 | RSVTPCHYS |
| 33791 | RSVTPCHYT |
| 33792 | RSVTPCHYY |
| 33793 | RTHCHAKAD |
| 33794 | RTHGNAKAD |
| 33795 | RTHGPAKAD |
| 33796 | RTSWKWEYY |
| 33797 | RVAVTNHRY |
| 33798 | RVCFKEDAF |
| 33799 | RVDATNHRY |
| 33800 | RVDVTNHRF |
| 33801 | RVEPTTVAD |
| 33802 | RVESQWDGC |
| 33803 | RVQSQWHGC |
| 33804 | RWFLSIDYR |
| 33805 | SADPRVDCP |
| 33806 | SADSETPWF |
| 33807 | SCWLITKTE |
| 33808 | SEHCYRHQH |
| 33809 | SEWMIWKCR |
| 33810 | SGCFWRHTS |
| 33811 | SGCFWRNAS |
| 33812 | SGCFWRNTP |
| 33813 | SGCFWRSTS |
| 33814 | SGCFWRTTS |
| 33815 | SGIRIFLFG |
| 33816 | SHCLYWMRH |
| 33817 | SHCMYWMKH |
| 33818 | SHCVYWMKH |
| 33819 | SHDVRMHML |
| 33820 | SHFLYWMKH |
| 33821 | SIVCRVNYP |
| 33822 | SKCGFDVAD |
| 33823 | SKCGFDVAG |
| 33824 | SKCGFDVAP |
| 33825 | SKCGFDVAV |
| 33826 | SKCGFNVAA |
| 33827 | SKCSFDVAA |
| 33828 | SKRDMDPAP |
| 33829 | SLGNRNADY |
| 33830 | SLQNRNADY |
| 33831 | SLRDRNADY |
| 33832 | SMDTSDWWE |
| 33833 | SQWMIWKCQ |
| 33834 | SVCPNRYAD |
| 33835 | SVCPTRHAD |
| 33836 | SVCPTRYAE |
| 33837 | SVCPTRYAH |
| 33838 | SVCPTRYAN |
| 33839 | SVRNRNADY |
| 33840 | TAAWRTDDR |
| 33841 | TADPRVDCP |
| 33842 | TADRWFDAS |
| 33843 | TADRWGDAS |
| 33844 | TAELRTDDR |
| 33845 | TAEPKMDHE |
| 33846 | TAEWRNDDR |
| 33847 | TAEWRTDAR |
| 33848 | TAEWRTDDW |
| 33849 | TAEWRTDYR |
| 33850 | TAGWRTDDR |
| 33851 | TAKWRTDDR |
| 33852 | TCILRQSPT |
| 33853 | TCLLRQSQT |
| 33854 | TCPRKDDDM |
| 33855 | TCTRKDDDK |
| 33856 | TCTRKDDDT |
| 33857 | TCWLITKTE |
| 33858 | TFVCRVNYP |
| 33859 | TGDRFMVDQ |
| 33860 | TGGRIGDSQ |
| 33861 | TIVCRINYP |
| 33862 | TIVCRVDYP |
| 33863 | TIVCRVHYP |
| 33864 | TIVCRVNYH |
| 33865 | TIVCRVNYR |
| 33866 | TIVCRVNYS |
| 33867 | TKKKLQCES |
| 33868 | TKKKMQCEA |
| 33869 | TKKKMQCEY |
| 33870 | TKKKMQCQS |
| 33871 | TKQKMQCES |
| 33872 | TLVCRVNYP |
| 33873 | TMKLGDMDG |
| 33874 | TSEWRTDDR |
| 33875 | TVAMVMDFC |
| 33876 | TVEHNRLAN |
| 33877 | TVVCRVNYP |
| 33878 | VAAQSDDRE |
| 33879 | VAQQIRDFH |
| 33880 | VAQRTTDHR |
| 33881 | VCMHLRQGG |
| 33882 | VCMHVRQGA |
| 33883 | VCMPVRQGG |
| 33884 | VCWLITKTE |
| 33885 | VCWMITKTE |
| 33886 | VCWVITKTE |
| 33887 | VDDKHITGY |
| 33888 | VDDKNITCY |
| 33889 | VDDKYITCY |
| 33890 | VEDKHITCY |
| 33891 | VEPFPCDWS |
| 33892 | VGGDYFRSD |
| 33893 | VKILSEMMT |
| 33894 | VKLLSEMMN |
| 33895 | VKMLSEMMN |
| 33896 | VPVDNLWND |
| 33897 | VVAVIRKMQ |
| 33898 | VVCMNHCDM |
| 33899 | VVEHNRLAN |
| 33900 | VVEPYTDHQ |
| 33901 | VVQQIRDEN |
| 33902 | VVQQIRDFP |
| 33903 | VVQQIRDFR |
| 33904 | VVQQLRDFH |
| 33905 | VVQQSRDFH |
| 33906 | VWECPRVGS |
| 33907 | WAEYRKTHA |
| 33908 | WAEYRKTHH |
| 33909 | WAEYRQTHD |
| 33910 | WAIRNMELR |
| 33911 | WAKYRKTHD |
| 33912 | WEHRFPASM |
| 33913 | WFAARQHFP |
| 33914 | WFIARQHFP |
| 33915 | WFVARKHFP |
| 33916 | WFVARQHFA |
| 33917 | WFVARQHFT |
| 33918 | WFVARQNFP |
| 33919 | WFVGRQHFP |
| 33920 | WGIRNMELG |
| 33921 | WGIRNMELQ |
| 33922 | WGIRNMEPR |
| 33923 | WGIRNMGLR |
| 33924 | WGIRNMKLR |
| 33925 | WGIRNMVLR |
| 33926 | WGIRNTELR |
| 33927 | WGNRNMELR |
| 33928 | WGSRNMELR |
| 33929 | WGVRNMELR |
| 33930 | WLVCRCAWE |
| 33931 | WSAKSTKKQ |
| 33932 | WSAKTTKEQ |
| 33933 | WSIRNMELR |
| 33934 | WVVCRCDWE |
| 33935 | WVVCRCSWE |
| 33936 | WVVCRCTWE |
| 33937 | YAARFACYQ |
| 33938 | YACAIQQHL |
| 33939 | YADRFAGYQ |
| 33940 | YANLGREHN |
| 33941 | YAPDRDGCD |
| 33942 | YDEQWNDNS |
| 33943 | YDIMNISPF |
| 33944 | YDIMTISPC |
| 33945 | YDMMTISPF |
| 33946 | YENLAREHN |
| 33947 | YFASWIRNH |
| 33948 | YFNSWIRNH |
| 33949 | YFQSMGHMW |
| 33950 | YFRAMGHMW |
| 33951 | YFTGWIRNH |
| 33952 | YGCFWRNTS |
| 33953 | YHCQNYVAS |
| 33954 | YIALRATFQ |
| 33955 | YIDKIAGWL |
| 33956 | YIGLRATFE |
| 33957 | YIGLRATFK |
| 33958 | YIGLRATFP |
| 33959 | YIGVRATFQ |
| 33960 | YINKIAGWW |
| 33961 | YIPDRDGCD |
| 33962 | YIVLRATFQ |
| 33963 | YKCAHDPNG |
| 33964 | YKCAHDPTE |
| 33965 | YKCAYDPTG |
| 33966 | YKCGHDPTG |
| 33967 | YKCPHDPTG |
| 33968 | YKCSHDPTG |
| 33969 | YKCTHDPTG |
| 33970 | YKCVHDPTG |
| 33971 | YKFDMPSFF |
| 33972 | YKGAHDPTG |
| 33973 | YKGGYGCHE |
| 33974 | YLGLRATFQ |
| 33975 | YQDGRCYKE |
| 33976 | YSGQHFRTL |
| 33977 | YTMCKANWN |
| 33978 | YVHDRDGCD |
| 33979 | YVPDRAGCD |
| 33980 | YVPDRDGCA |
| 33981 | YVPDRDGCC |
| 33982 | YVPDRDGCG |
| 33983 | YVPDRDGYD |
| 33984 | YVPDRDVCD |
| 33985 | YVPDRNGCD |
| 33986 | YVPDRVGCD |
| 33987 | YVPYRDGCD |
| 33988 | YVRDRDGCD |
| 33989 | YVTDRDGCD |
| 33990 | YYCRRTFHH |

### Example 41

AAV5 Variants with Spinal Cord Tissue Tropism as Identified by Machine Learning

This example describes preferred properties of engineered AAV5 variants that display spinal cord tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display spinal cord tissue tropism.

Favored biophysical properties and favored amino acid residues at each position in the 581 to 589 region of an AAV5 VP capsid polypeptide, which are associated with a higher probability of spinal cord tissue tropism are described below. Any of the below machine learning-derived positional amino acid preferences described in TABLE 66 can be present alone or in combination with each other or in combination with any of the amino acid preferences observed and described in EXAMPLE 10, TABLE 19. Listed below in TABLE 66 are a summary of positional features shared between the top important features for spinal cord tropism extracted from the ML models.

TABLE 66
Machine Learning-Derived Spinal Cord Tissue Tropism Rules

High volume at Xaa1
Xaa1 is selected from F, W, or Y
Low mutability at Xaa1
Xaa1 is selected from Y, F, L, or C
High solubility at Xaa1
Xaa1 is selected from W, F, I, or L
Low average flexibility at Xaa1
Xaa1 is selected from F, M, or W
Low hydropathy at Xaa2
Xaa2 is selected from P or Y
Low hydrophilicity at Xaa3
Xaa3 is selected from Y, W, V, M, F, I, or L
High solubility at Xaa3
Xaa3 is selected from W, F, I, or L
High volume at Xaa6
Xaa6 is selected from W, R, K, M, I, or L
High mol mass at Xaa6
Xaa6 is selected from W
High mol mass at Xaa8
Xaa8 is selected from W, E, K, M, H, or Q
High volume at Xaa8
Xaa8 is selected from W, K, M, I, or L
High goldman engelman steitz at Xaa8
Xaa8 is selected from V or L
High hydropathy at Xaa9
Xaa9 is selected from V or I
High solubility at Xaa9
Xaa9 is selected from W, F, I, or L

TABLE 67 below provides sequences of the 581 to 589 region for variant AAV VP1 capsid polypeptides that exhibited spinal cord tissue tropism and comport to one or more of the rules provided in TABLE 66. The present disclosure, thus, provides for rAAVs composed of engineered AAV5 VP1 capsid polypeptides having a SEQ ID NO: 2, wherein the Xaa1 to Xaa9 region is any one of SEQ ID NO: 35438-SEQ ID NO: 36437, as disclosed in TABLE 67. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 67
Sequences of the 581 to 589 Region in AAV5 VP1
Capsid Polypeptide that Drive Spinal Cord Tissue
Tropism

| SEQ | |
|---|---|
| ID | 581-589 |
| NO | Sequence |
| 35438 | ACLLFLVGI |
| 35439 | ACYWLLNWF |
| 35440 | ACYWWLNWF |
| 35441 | ACYWWMNWF |
| 35442 | ADHPENFSD |
| 35443 | ADWLRRNIW |
| 35444 | AEFRPKISR |
| 35445 | AFIQQNNQD |
| 35446 | AFIQQSDQD |
| 35447 | AFIQQSNED |
| 35448 | AFIQQSTQD |
| 35449 | AFIQRSNQD |
| 35450 | AFVQQSNQD |
| 35451 | AFWYSMAFP |
| 35452 | AGDRTISMW |
| 35453 | AHFRAYTQY |
| 35454 | AHKWFCEGS |
| 35455 | AHKWVCEDS |
| 35456 | AKNVFEEHV |
| 35457 | ALFAPNGEV |
| 35458 | ALFYMEKMP |
| 35459 | AMVLFLVDI |
| 35460 | AMWIRGIWG |
| 35461 | ANDMCNYYG |
| 35462 | AQGNKTYVI |
| 35463 | AQKKCLSFG |
| 35464 | AQKWFCEDS |
| 35465 | AQQKCLSFG |
| 35466 | AQVNKTYLI |
| 35467 | AQVNKTYVI |
| 35468 | AQVTKTYVI |
| 35469 | ATMFSVIHQ |
| 35470 | ATWVHYDMY |
| 35471 | AVIQQSNQD |
| 35472 | AWFDMEKMP |
| 35473 | AWFHMEKMP |
| 35474 | AWFNMEKMP |
| 35475 | AWFYKEKMP |
| 35476 | AWFYMEEMP |
| 35477 | AWFYMEKMS |
| 35478 | AWFYMENMP |
| 35479 | AWFYMGKMP |
| 35480 | AWFYREKMP |
| 35481 | AWLQGGIYN |
| 35482 | AWSYMEKMP |
| 35483 | CAVKQECSR |
| 35484 | CCCSEIGVN |
| 35485 | CHVMSVPCA |
| 35486 | CKNWYINMH |
| 35487 | CQWTPEIFQ |
| 35488 | CTQCYGDMP |
| 35489 | CWAEFRMRD |
| 35490 | CWGEFRMRV |
| 35491 | CWTEFRMRV |
| 35492 | CWVEFRMRV |
| 35493 | CWWTPEIFQ |
| 35494 | DAKSPCDSV |
| 35495 | DAKSPCESF |
| 35496 | DAVNDLTWP |
| 35497 | DCIPDWSST |
| 35498 | DCYWWLNWF |
| 35499 | DEANKTYVI |
| 35500 | DEDNKTYVI |
| 35501 | DEVNKTYVI |
| 35502 | DFILDWSST |
| 35503 | DFIPDWSSI |
| 35504 | DFIRDWSST |
| 35505 | DFITDWSST |
| 35506 | DFVPDWSST |
| 35507 | DFWYGMAFP |
| 35508 | DFWYSMACP |
| 35509 | DFWYSMAVP |
| 35510 | DFWYSMTFP |
| 35511 | DFWYSRAFP |
| 35512 | DFWYSVAFP |
| 35513 | DFWYTMAFP |
| 35514 | DHVNKPYVI |
| 35515 | DIIPDWSST |
| 35516 | DIRIVTDGR |
| 35517 | DIWYSMAFP |
| 35518 | DKGNKTYVI |
| 35519 | DKNVFEEDV |
| 35520 | DKNVFEQHV |
| 35521 | DKNVYEEHV |
| 35522 | DNVNKTYVI |
| 35523 | DPGNKTYVI |
| 35524 | DPVNKTYVL |
| 35525 | DPVNKTYVV |
| 35526 | DPVTKTYVI |
| 35527 | DQAEVLSDH |
| 35528 | DQANKTYVI |
| 35529 | DQDNKTYVI |
| 35530 | DQDTKTYVI |
| 35531 | DQGNKTYVI |
| 35532 | DQGSKTYVI |
| 35533 | DQINKPYVI |
| 35534 | DQINKTYVI |
| 35535 | DQINKTYVS |
| 35536 | DQLNKTYVI |
| 35537 | DQVDKTYVI |
| 35538 | DQVNKNYVI |
| 35539 | DQVNKPYEI |
| 35540 | DQVNKTHVI |
| 35541 | DQVNKTNVI |
| 35542 | DQVNKTSVI |
| 35543 | DQVNKTYAI |
| 35544 | DQVNKTYEI |
| 35545 | DQVNKTYML |
| 35546 | DQVNKTYVF |
| 35547 | DQVNKTYVN |
| 35548 | DQVNKTYVV |
| 35549 | DQVNMTYVI |
| 35550 | DQVNNTYMI |
| 35551 | DQVNQTYVI |
| 35552 | DQVNRTYVI |
| 35553 | DQVSKTYVI |
| 35554 | DQVTKTYVI |
| 35555 | DQVTQTYVI |
| 35556 | DRNVFEEHV |
| 35557 | DRVNKTYLI |
| 35558 | DRVNKTYVS |
| 35559 | DVNLKITTH |
| 35560 | DVNLQIATH |
| 35561 | DVYYCPEAI |
| 35562 | DWMQGGIYH |
| 35563 | DWMQGGIYY |
| 35564 | DYAQYYRTW |
| 35565 | DYDPDIKDP |
| 35566 | DYDPDLKDQ |
| 35567 | DYGPDIKDQ |
| 35568 | ECLLFLIGI |
| 35569 | ECMLFLVGI |
| 35570 | ECVSKWIVF |
| 35571 | EDWIRRNIW |
| 35572 | EDWLRCNIW |
| 35573 | EDWLRRDIW |
| 35574 | EFIQQSNQD |
| 35575 | EHFRAYAQY |
| 35576 | EHFRAYIQY |
| 35577 | EHFRDYTQY |
| 35578 | EHFRSYTQY |
| 35579 | EHWLRRNIW |
| 35580 | EIIPQFAYF |
| 35581 | EINREVREN |
| 35582 | EINRVVRQN |
| 35583 | EISREVRQN |
| 35584 | EKELKNSTQ |
| 35585 | ELIYKFKTD |
| 35586 | ELIYKIETD |
| 35587 | ELIYKMKTD |
| 35588 | ELVNKTYVI |
| 35589 | ELVYKIKTD |
| 35590 | EMINACFQT |
| 35591 | EMKCIGAAI |
| 35592 | EMKCMGAGI |
| 35593 | EMKCTGAGI |
| 35594 | EMKWLGAGI |
| 35595 | EMKYIGAGI |
| 35596 | EMWIGGIWG |
| 35597 | EMWIRGVWG |
| 35598 | EMWVRGVWG |
| 35599 | EQEIKNSTQ |
| 35600 | EQELKDSTQ |
| 35601 | EQELKNSTL |
| 35602 | EQELKTSTQ |
| 35603 | EQEVKNSTQ |
| 35604 | EQQLKNSTQ |
| 35605 | ERFRAYTQY |
| 35606 | ETFHNTATI |
| 35607 | ETWVHYDMY |
| 35608 | EYGTKVKMD |
| 35609 | EYSTKVKMD |
| 35610 | EYVTKVKMD |
| 35611 | FAEASITRW |
| 35612 | FASAFANTD |
| 35613 | FASAFSNSD |
| 35614 | FASAFVNSD |
| 35615 | FASVFANSD |
| 35616 | FCMEWEVMD |
| 35617 | FFMAWEVMD |
| 35618 | FFMDWEVMD |
| 35619 | FFMEWEGMD |
| 35620 | FFMEWELMD |
| 35621 | FFMEWEVKD |
| 35622 | FFMEWEVRD |
| 35623 | FFMEWEVTD |
| 35624 | FFMEWEVVD |
| 35625 | FFMEWKVMD |
| 35626 | FFMEWQVMD |
| 35627 | FFYECYSNK |
| 35628 | FIIGCMLIW |
| 35629 | FIIHCMLIW |
| 35630 | FIIRAMLIW |
| 35631 | FIIRCMLIL |
| 35632 | FIIRCMLVW |
| 35633 | FIIRCMVIW |
| 35634 | FIIRCVLIW |
| 35635 | FIIRCVLVW |
| 35636 | FIIRGMLIW |
| 35637 | FIIRWMLIW |
| 35638 | FILRCLLIW |
| 35639 | FILRCMLIC |
| 35640 | FILRCMLIW |
| 35641 | FIVGGLAFA |
| 35642 | FIVRCMLIW |
| 35643 | FKFISYIGD |
| 35644 | FKHWYINMH |
| 35645 | FKICFSPTV |
| 35646 | FKNGYINMH |
| 35647 | FKNHYEGGP |
| 35648 | FKNLYINMH |
| 35649 | FKNWFINMH |
| 35650 | FKNWHINMH |
| 35651 | FKNWSINMH |
| 35652 | FKNWYIHMH |
| 35653 | FKNWYINMD |
| 35654 | FKNWYINMF |
| 35655 | FKNWYINML |
| 35656 | FKNWYINMN |
| 35657 | FKNWYINMP |
| 35658 | FKNWYINMR |
| 35659 | FKNWYINMY |
| 35660 | FKNWYINRH |
| 35661 | FKNWYINTH |
| 35662 | FKNWYINVH |
| 35663 | FKNWYISMH |
| 35664 | FKNWYLHMH |
| 35665 | FKNWYNNMH |
| 35666 | FKNWYVNMH |
| 35667 | FKSWYINMH |
| 35668 | FKVGGLAFA |
| 35669 | FKYWYINMH |
| 35670 | FLIRCMLIW |
| 35671 | FMIRCMLIW |
| 35672 | FPEASLTRW |
| 35673 | FQNWYINMH |
| 35674 | FRNWYINMH |
| 35675 | FRNWYMNMH |
| 35676 | FTFGGLAFA |
| 35677 | FTIGGLAFA |
| 35678 | FTNTKKQAF |
| 35679 | FTNWYINMD |
| 35680 | FTNWYINMH |
| 35681 | FTNWYINMN |
| 35682 | FTVAGLAFA |
| 35683 | FTVGGLAFG |
| 35684 | FTVGGLAFP |
| 35685 | FTVGGLAFT |
| 35686 | FTVGGLAFV |
| 35687 | FTVGGLGFA |
| 35688 | FTVGGLSFA |
| 35689 | FTVGGLTFA |
| 35690 | FTVGGMAFA |
| 35691 | FWCHIIGIY |
| 35692 | FWCNIIGIY |
| 35693 | FWGNIIGIY |
| 35694 | FWSNFIGIY |
| 35695 | FWSNIIGFY |
| 35696 | FWSNIIGIC |
| 35697 | FWSNIIGIH |
| 35698 | FWSNIIGIN |
| 35699 | FWSNIIGLY |
| 35700 | FWSNIIGMY |
| 35701 | FWSNIIGVY |
| 35702 | FWSNIMGIY |
| 35703 | FWSNLIGIY |
| 35704 | FWSNMIGIY |
| 35705 | FWSNVIGIY |
| 35706 | FWSSIIGIY |
| 35707 | FWSTIIGIY |
| 35708 | FWTNIIGIY |
| 35709 | FYMEWEVMD |
| 35710 | GCYWWLNWF |
| 35711 | GDEYYDIMG |
| 35712 | GDHTENFSD |
| 35713 | GEFRPKISR |
| 35714 | GFIQQSNQD |
| 35715 | GHVMTVPCA |
| 35716 | GIKYKDILS |
| 35717 | GINREVRQN |
| 35718 | GKNVFEEHV |
| 35719 | GQGNKTYVI |
| 35720 | GSASRNQRD |
| 35721 | GSSPGKGWM |
| 35722 | GSSSRNQRD |
| 35723 | GTWVHYDMF |
| 35724 | GWAEFRMRV |
| 35725 | GWAEFRVRV |
| 35726 | GWSEFRMRV |
| 35727 | HPVNKTYVI |
| 35728 | HQGNKTYVI |
| 35729 | HRFQEKGSR |
| 35730 | HRFQEKGTH |
| 35731 | HRFREKGSH |
| 35732 | HTLLWYGIW |
| 35733 | HTLLWYGIY |
| 35734 | HVYCCPEAI |
| 35735 | ICHCCMAWG |
| 35736 | ICHCCMVWG |
| 35737 | ICHCCMYWG |
| 35738 | ICHWCMDWG |
| 35739 | ICYCCMDWG |
| 35740 | ICYWWLNCF |
| 35741 | ICYWWLNWF |
| 35742 | IDRWWVIDV |
| 35743 | IHIGTNSYA |
| 35744 | IHTATNSYA |
| 35745 | IHTGTNSYV |
| 35746 | IIYECYSNK |
| 35747 | IKHIAAESR |
| 35748 | IKHIATETR |
| 35749 | IKHISTESR |
| 35750 | IKNWYINMH |
| 35751 | IKNWYINMP |
| 35752 | IKSQTLTDC |
| 35753 | IMFHCREMG |
| 35754 | IMVHCRQMG |
| 35755 | IMVHCRVMG |
| 35756 | IRNMFEQYT |
| 35757 | IRSHTLTYC |
| 35758 | IRSQTITDC |
| 35759 | IRSQTLTDW |
| 35760 | IRSQTLTDY |
| 35761 | IRSQTLTNC |
| 35762 | IRSQTVTDC |
| 35763 | IRSRTLTDC |
| 35764 | IRWLRLHKK |
| 35765 | ISWIIRADI |
| 35766 | ISWINRADI |
| 35767 | ISWITRADV |
| 35768 | ISWITRAGI |
| 35769 | ISWITRTDI |
| 35770 | ITSQTLTDC |
| 35771 | ITWLHYDMY |
| 35772 | IWFYMEKMP |
| 35773 | IWSNIIGIY |
| 35774 | IWYWWLNWF |
| 35775 | KAVWDPSSM |
| 35776 | KCWDCHDAG |
| 35777 | KCWDCHEGG |
| 35778 | KEDFGWKHV |
| 35779 | KESRSQTGD |
| 35780 | KEVFGWKHM |
| 35781 | KGVWDPGSM |
| 35782 | KGVWDPSST |
| 35783 | KHIGSYTQF |
| 35784 | KHNMLYHMD |
| 35785 | KHYKWGHGQ |
| 35786 | KINREVRQN |
| 35787 | KKWSCHRGH |
| 35788 | KKYQDTHTF |
| 35789 | KMWIRGIWG |
| 35790 | KRIKMPPPR |
| 35791 | KRIKMPPTK |
| 35792 | KWHAGPGVV |
| 35793 | KWHCGPGVV |
| 35794 | KWHGGPGAV |
| 35795 | KWHGGPGDV |
| 35796 | KWHGGPGFV |
| 35797 | KWHGGPGVM |
| 35798 | KWHGGQGVV |
| 35799 | KWHGGRGVV |
| 35800 | KWLGGPGVV |
| 35801 | KWLYGWPIK |
| 35802 | KWYGGPGVV |
| 35803 | KYNMMYHMD |
| 35804 | KYNMRYHMD |
| 35805 | KYNMVYHMD |
| 35806 | KYNVLYHMD |
| 35807 | KYSMLYHMD |
| 35808 | LATMSLTWW |
| 35809 | LCYWWLNWF |
| 35810 | LEKWGHMHT |
| 35811 | LKICFGPTV |
| 35812 | LKIGFSPTV |
| 35813 | LPEIYLSFC |
| 35814 | LPKRYLSFC |
| 35815 | LPQIYLSFC |
| 35816 | LPTIYLSFC |
| 35817 | LQICFSPTV |
| 35818 | LRICFSPTV |
| 35819 | LRWVRLHKK |
| 35820 | LSKIYLSFC |
| 35821 | LSSCYYTTY |
| 35822 | LSWITRADI |
| 35823 | LVFLRGQHY |
| 35824 | LWFYMEEMP |
| 35825 | LWFYMEKMP |
| 35826 | LWSNMIGIY |
| 35827 | MFRIEAVCP |
| 35828 | MKFQDTHTF |
| 35829 | MKRDAAFCA |
| 35830 | MKREAAVCA |
| 35831 | MKRETAFCP |
| 35832 | MKRETAFCS |
| 35833 | MKRETAFCT |
| 35834 | MKRETAVCA |
| 35835 | MKRETGFCA |
| 35836 | MKYKDTHTF |
| 35837 | MKYQDTHTC |
| 35838 | MKYQDTHTI |
| 35839 | MKYWDTHTF |
| 35840 | MYFFVWRFM |
| 35841 | MYIFGWRFM |
| 35842 | MYIFVWGFM |
| 35843 | MYIFVWRFT |
| 35844 | MYIFVWRIM |
| 35845 | MYIFVWRVM |
| 35846 | MYIFYWRFM |
| 35847 | MYIIVWRFM |
| 35848 | MYISVWRFM |
| 35849 | MYIVVWRFM |
| 35850 | MYIYFWRFM |
| 35851 | MYIYVWRFM |
| 35852 | MYLFVWRFM |
| 35853 | NANDVQWYD |
| 35854 | NAPDVQWYD |
| 35855 | NATDFQWYD |
| 35856 | NATDGQWHD |
| 35857 | NATDVEWYD |
| 35858 | NATDVKWYD |
| 35859 | NATDVLWYD |
| 35860 | NATDVRWYD |
| 35861 | NCDRSLAEY |
| 35862 | NCHIDRVQD |
| 35863 | NCHVDHVQD |
| 35864 | NCKNLTYYI |
| 35865 | NFIPDWSST |
| 35866 | NFWYSMAFP |
| 35867 | NIDAHERYR |
| 35868 | NKNVFEEHV |
| 35869 | NLCRHLFQC |
| 35870 | NLCRHLFQF |
| 35871 | NNYKFQQDG |
| 35872 | NNYKGQQDG |
| 35873 | NNYKLQQDG |
| 35874 | NNYKVQEDG |
| 35875 | NPAYGRGHI |
| 35876 | NPELGKCEI |
| 35877 | NPGWRYMWF |
| 35878 | NPGWRYMWW |
| 35879 | NQAYGRGHI |
| 35880 | NQVNKTYVI |
| 35881 | NRAYGRGHV |
| 35882 | NSWITRADI |
| 35883 | NSWRHICDH |
| 35884 | NVAAHERYR |
| 35885 | NVCRHLFQF |
| 35886 | NVDAHERYK |
| 35887 | NVDAPERYR |
| 35888 | NVDVHERYR |
| 35889 | NVGAHERYR |
| 35890 | NVYYCPEAI |
| 35891 | NWCKHLFQF |
| 35892 | NWCRDLFQF |
| 35893 | NWCRHLFEC |
| 35894 | NWCRHLFEF |
| 35895 | NWCRHLFKF |
| 35896 | NWCRHLFKY |
| 35897 | NWCRHLFQC |
| 35898 | NWCRHLFQI |
| 35899 | NWCRHLFQV |
| 35900 | NWCRHLFQY |
| 35901 | NWCRHLFRF |
| 35902 | NWCRHLIQF |
| 35903 | NWCRHLVQF |
| 35904 | NWCRHRFQF |
| 35905 | NWCRILFQF |
| 35906 | NWCRLLFQF |
| 35907 | NWCRNLFQF |
| 35908 | NWCRQLFQV |
| 35909 | NWCRVLFHF |
| 35910 | NWFRHLFQF |
| 35911 | NWGRHLFQF |
| 35912 | NWWRHLFQF |
| 35913 | NYAQHYRTW |
| 35914 | NYSQYYRTW |
| 35915 | PADRTISMW |
| 35916 | PDDRTISMW |
| 35917 | PGDRTISKW |
| 35918 | PGDRTITMW |
| 35919 | PGDRTLSMW |
| 35920 | PGNRTISMW |
| 35921 | PHKWFCEDS |
| 35922 | PPDIGTQFI |
| 35923 | PPHIGTEFI |
| 35924 | PPHIGTKFI |
| 35925 | PPHLGTQFI |
| 35926 | PPHMGTQFI |
| 35927 | PPHTGTQFI |
| 35928 | PPLIGTQFI |
| 35929 | PRFQEKGSH |
| 35930 | PRHIGTQFI |
| 35931 | PWFYLEKMP |
| 35932 | PWFYMEEMP |
| 35933 | QCLLFLVGI |
| 35934 | QEKWGHKHT |
| 35935 | QEKWGHMHA |
| 35936 | QEKWGHVHT |
| 35937 | QTIIGNVWV |
| 35938 | QWFYMEKMP |
| 35939 | RAHWSDIAD |
| 35940 | RAPADWACA |
| 35941 | RDGSNWLCI |
| 35942 | RDPADWACG |
| 35943 | RDPADWACS |
| 35944 | RDPADWTCA |
| 35945 | RDPADWVCA |
| 35946 | RDPSDWACA |
| 35947 | RDPTDWACA |
| 35948 | RDTADWACA |
| 35949 | REDFGWKHM |
| 35950 | RHIGSYTQF |
| 35951 | RHIHRYWEC |
| 35952 | RHKWFCEAS |
| 35953 | RHPADWACA |
| 35954 | RHVHPYWEC |
| 35955 | RHVHQYWEC |
| 35956 | RHVHRFWEC |
| 35957 | RHVHRYWVC |
| 35958 | RIMHSWYND |
| 35959 | RKEQDHWDK |
| 35960 | RKVESYCIT |
| 35961 | RKWSCHRAH |
| 35962 | RKWSCHRCH |
| 35963 | RKWSCHRGL |
| 35964 | RKWSCHRGY |
| 35965 | RKWSCHRVH |
| 35966 | RKWSCNRGH |
| 35967 | RKWYCHRGH |
| 35968 | RPHIGTQFI |
| 35969 | RPVQEDGCG |
| 35970 | RQNDTHRAN |
| 35971 | RQVQRYWEC |
| 35972 | RQWSCHRGH |
| 35973 | RRFQEKGSH |
| 35974 | RRIESYCIT |
| 35975 | RRIKMPPPK |
| 35976 | RTVKEDGCG |
| 35977 | RVPADWACA |
| 35978 | RWSYMKAGK |
| 35979 | RWSYMQAGI |
| 35980 | RWSYMQTGK |
| 35981 | RWSYMQVGK |
| 35982 | RWSYMRAGK |
| 35983 | RYIFVWRLM |
| 35984 | RYNMLYHMD |
| 35985 | RYPADWACA |
| 35986 | SAAWQFGES |
| 35987 | SAAWQFGVS |
| 35988 | SDDMCNYYG |
| 35989 | SDMIKLAEI |
| 35990 | SGADHYHLK |
| 35991 | SGFYMEMMP |
| 35992 | SGFYMEQMP |
| 35993 | SGFYMERMP |
| 35994 | SHKWFCEDS |
| 35995 | SHYWPMCWS |
| 35996 | SLFYMDKMP |
| 35997 | SLFYMEKML |
| 35998 | SLFYMEKMP |
| 35999 | SLLRFQPSI |
| 36000 | SLMLFLPSI |
| 36001 | SLMRCLPSI |
| 36002 | SLMRFLASI |
| 36003 | SLMRFLHSI |
| 36004 | SLMRFLPAI |
| 36005 | SLMRFLPST |
| 36006 | SLMRFLPSV |
| 36007 | SLMRFLPYI |
| 36008 | SLMRFLRSI |
| 36009 | SLMRFLSSI |
| 36010 | SLMRFLTSI |
| 36011 | SLMRFPPSI |
| 36012 | SLMRFVPSI |
| 36013 | SLMRVLPSI |
| 36014 | SLVRFLLSI |
| 36015 | SLVRFLPSI |
| 36016 | SMHDSNFNV |
| 36017 | SMHDYNFNI |
| 36018 | SMHDYTFNV |
| 36019 | SNDKCNYYG |
| 36020 | SNDMCDYYG |
| 36021 | SNDMCNHYG |
| 36022 | SNDMCNSYG |
| 36023 | SNNMCNYYG |
| 36024 | SPYWLMRMG |
| 36025 | SQDCGRPEI |
| 36026 | SRMRFLPSI |
| 36027 | SRMRLLPSI |
| 36028 | SSASVNHTS |
| 36029 | SSDMCNYYG |
| 36030 | SSFYMEKMP |
| 36031 | SSSPGKGWK |
| 36032 | SSWITRADI |
| 36033 | STDMCNYYG |
| 36034 | SVADHYHRK |
| 36035 | SVADHYNLK |
| 36036 | SVADYYHLK |
| 36037 | SVAHHYHLK |
| 36038 | SVARQMHSW |
| 36039 | SVMRFLPSI |
| 36040 | SVSDHYHLK |
| 36041 | SWCFMEKMP |
| 36042 | SWCRHLFEF |
| 36043 | SWCRHLFQF |
| 36044 | SWCYIEKMP |
| 36045 | SWCYMEEMP |
| 36046 | SWCYMEQMP |
| 36047 | SWCYMVKMP |
| 36048 | SWFCVEKMP |
| 36049 | SWFDMEKMP |
| 36050 | SWFFMEKMP |
| 36051 | SWFFMVKMP |
| 36052 | SWFFVEKMP |
| 36053 | SWFHMEKMP |
| 36054 | SWFHMEKMQ |
| 36055 | SWFNMEQMP |
| 36056 | SWFSKEKMP |
| 36057 | SWFSVEKMP |
| 36058 | SWFYIERMP |
| 36059 | SWFYKETMP |
| 36060 | SWFYMAQMP |
| 36061 | SWFYMDKMT |
| 36062 | SWFYMDMMP |
| 36063 | SWFYMEAMP |
| 36064 | SWFYMEDMP |
| 36065 | SWFYMEEVP |
| 36066 | SWFYMEGMP |
| 36067 | SWFYMEKIR |
| 36068 | SWFYMEKMA |
| 36069 | SWFYMEKMI |
| 36070 | SWFYMEKMR |
| 36071 | SWFYMEKMT |
| 36072 | SWFYMEKRP |
| 36073 | SWFYMEKVP |
| 36074 | SWFYMEMMP |
| 36075 | SWFYMERMP |
| 36076 | SWFYMETMP |
| 36077 | SWFYMEWMP |
| 36078 | SWFYMKKMP |
| 36079 | SWFYMQKMP |
| 36080 | SWFYMVKMP |
| 36081 | SWFYRDKMP |
| 36082 | SWFYREKLP |
| 36083 | SWFYVEEMP |
| 36084 | SWFYVEKIP |
| 36085 | SWFYVEKMP |
| 36086 | SWIYMDKMP |
| 36087 | SWIYMEKMT |
| 36088 | SWIYMEQMP |
| 36089 | SWTYMEKMP |
| 36090 | SWVCMEKMP |
| 36091 | SWVYMEKMQ |
| 36092 | SWVYMEKMS |
| 36093 | SWVYMEMMP |
| 36094 | SWVYMEQMP |
| 36095 | SWVYMKKMP |
| 36096 | SWYCMEKMP |
| 36097 | SWYYDERSN |
| 36098 | SWYYMEEMP |
| 36099 | SWYYMEKMP |
| 36100 | TAEKFICFP |
| 36101 | TCARSLAEY |
| 36102 | TCDRSLAQY |
| 36103 | TCDRSLGEY |
| 36104 | TCDRSLPEY |
| 36105 | TCDRSLTEY |
| 36106 | TCDRSLVEY |
| 36107 | TCDRTLAEY |
| 36108 | TCKNLTYSI |
| 36109 | TCKNMTYYI |
| 36110 | TCKSLTYYI |
| 36111 | TCNRSLAEY |
| 36112 | TCRNLTYYI |
| 36113 | TCTNLTYYI |
| 36114 | TDKWFCVDS |
| 36115 | THIASYTQF |
| 36116 | THICSYTQF |
| 36117 | THIGSYTEF |
| 36118 | THIGTYTQF |
| 36119 | THIVSYTQF |
| 36120 | THKWCCEDS |
| 36121 | THKWFCDYS |
| 36122 | THKWFCEDC |
| 36123 | THKWFCEDI |
| 36124 | THKWFCEDN |
| 36125 | THKWFCEDT |
| 36126 | THKWFCEHS |
| 36127 | THKWFCENI |
| 36128 | THKWFCENS |
| 36129 | THKWFCEVS |
| 36130 | THKWFCEYS |
| 36131 | THKWFCGDI |
| 36132 | THKWFCQDS |
| 36133 | THKWFCVDS |
| 36134 | THKWICEDI |
| 36135 | THKWVCEDS |
| 36136 | THRWFCEDS |
| 36137 | THRWFCEES |
| 36138 | THRWLCEDS |
| 36139 | TLMRFLPSI |
| 36140 | TPKWFCEDN |
| 36141 | TPKWFCEDS |
| 36142 | TPKWVCEDS |
| 36143 | TRKWFCEDS |
| 36144 | TSERFICFP |
| 36145 | TTDHFICFP |
| 36146 | TTELYICFP |
| 36147 | TWCRHLFQF |
| 36148 | TWFHMEKMP |
| 36149 | TWFYMDKMP |
| 36150 | TWFYMEKMP |
| 36151 | TWFYMEKMQ |
| 36152 | TWFYMEQMP |
| 36153 | TWLCGWPIK |
| 36154 | TWLFGWPIK |
| 36155 | TWLGGCKSV |
| 36156 | TWLYGWPFK |
| 36157 | TWLYGWPIQ |
| 36158 | TWLYGWPIR |
| 36159 | TWLYGWPIT |
| 36160 | TWLYGWPVK |
| 36161 | TWLYGWSIK |
| 36162 | TWLYGWTIK |
| 36163 | TWMYGWPIK |
| 36164 | TWPVGCKSV |
| 36165 | TWRGGCKSV |
| 36166 | TYAQYYRTW |
| 36167 | VAFRPKISR |
| 36168 | VCCWWLNWF |
| 36169 | VCSWWLNWF |
| 36170 | VCYWWLDWF |
| 36171 | VCYWWLHWI |
| 36172 | VCYWWLIWF |
| 36173 | VCYWWLNLF |
| 36174 | VCYWWLNWC |
| 36175 | VCYWWLNWI |
| 36176 | VCYWWLNWY |
| 36177 | VCYWWLSWL |
| 36178 | VCYWWLTWF |
| 36179 | VCYWWMNWF |
| 36180 | VEFGPKISR |
| 36181 | VEFKPKISR |
| 36182 | VEFRHKISR |
| 36183 | VEFRPEISR |
| 36184 | VEFRPKFSR |
| 36185 | VEFRPKLSR |
| 36186 | VEFRPKMSR |
| 36187 | VEFRPKTSR |
| 36188 | VEFRPKVSR |
| 36189 | VEFRPRISR |
| 36190 | VEFRPTISR |
| 36191 | VEFRRKISR |
| 36192 | VEFRSKISR |
| 36193 | VEFRTKISR |
| 36194 | VEFTPKISR |
| 36195 | VEYRPKISR |
| 36196 | VFIQQSNQD |
| 36197 | VFYWWLNWF |
| 36198 | VGYWWLNWF |
| 36199 | VIWVHYDMY |
| 36200 | VKFRPKISR |
| 36201 | VKKCHTCGA |
| 36202 | VKNWYINMH |
| 36203 | VKWVRLHKK |
| 36204 | VNWVHYDMY |
| 36205 | VQVNKTYVI |
| 36206 | VRWLRLHKK |
| 36207 | VRWVRIHKK |
| 36208 | VRWVRLHEK |
| 36209 | VRWVRLHKQ |
| 36210 | VRWVRLHKR |
| 36211 | VRWVRLHKT |
| 36212 | VRWVRLHRK |
| 36213 | VRWVRLHTK |
| 36214 | VRYWWLNCF |
| 36215 | VSWITRADI |
| 36216 | VTVGGLAFA |
| 36217 | VTWMHYDMY |
| 36218 | VTWVHYAMY |
| 36219 | VTWVHYDMF |
| 36220 | VTWVHYDMH |
| 36221 | VTWVHYNMY |
| 36222 | VTWVHYVMY |
| 36223 | VTWVHYYMY |
| 36224 | VVFLRDQHY |
| 36225 | VWSNIIGIY |
| 36226 | VWYWWLNWF |
| 36227 | VWYWWLNWL |
| 36228 | VYYWWLNWF |
| 36229 | WANFKTLNR |
| 36230 | WANFQTLNK |
| 36231 | WAPTPWRTH |
| 36232 | WCCKMWSWW |
| 36233 | WCMSQQYED |
| 36234 | WDGKFWETM |
| 36235 | WDIKFWETM |
| 36236 | WDLKFWETM |
| 36237 | WDMSQQYED |
| 36238 | WDNLRNTQI |
| 36239 | WDNSSNTQI |
| 36240 | WDNWINTQI |
| 36241 | WDNWSDTQI |
| 36242 | WDNWSHTQI |
| 36243 | WDNWSITQI |
| 36244 | WDNWSNTEI |
| 36245 | WDNWSNTKI |
| 36246 | WDNWSNTKK |
| 36247 | WDNWSNTLI |
| 36248 | WDNWSNTPI |
| 36249 | WDNWSNTQM |
| 36250 | WDNWSNTQV |
| 36251 | WDNWSNTRI |
| 36252 | WDNWSTTQI |
| 36253 | WDNWTNTQI |
| 36254 | WDNWTNTQM |
| 36255 | WDSWSNTQI |
| 36256 | WDVKFWESM |
| 36257 | WDVKYWETM |
| 36258 | WDVMFWETM |
| 36259 | WENWSNSQI |
| 36260 | WFCKKWSWW |
| 36261 | WFCKMLSWW |
| 36262 | WFCKMWGWG |
| 36263 | WFCKMWGWW |
| 36264 | WFCKMWIWW |
| 36265 | WFCKMWSGW |
| 36266 | WFCKMWSLW |
| 36267 | WFCKMWSWG |
| 36268 | WFCKMWSWL |
| 36269 | WFCKMWTWW |
| 36270 | WFCKVWSWW |
| 36271 | WFCNLWSWW |
| 36272 | WFCQMWSWW |
| 36273 | WFCRMWSWW |
| 36274 | WFCTLWSWW |
| 36275 | WFCTMWSWW |
| 36276 | WFFKMWSWW |
| 36277 | WFGKMWSWW |
| 36278 | WFNMPHDRH |
| 36279 | WFWKMWSWW |
| 36280 | WFYKMWSWW |
| 36281 | WGNWSNTQI |
| 36282 | WGVKFWETM |
| 36283 | WHCSVYTCS |
| 36284 | WHRDEMLTP |
| 36285 | WHREEMLTT |
| 36286 | WHVKFWETM |
| 36287 | WHWDEMLTT |
| 36288 | WIAAMTYWC |
| 36289 | WICKMWSWW |
| 36290 | WIHAMTYWC |
| 36291 | WIHMPHDRH |
| 36292 | WINMAHDRH |
| 36293 | WINMHHDRH |
| 36294 | WINMPDDRH |
| 36295 | WINMPHARH |
| 36296 | WINMPHDGH |
| 36297 | WINMPHDKH |
| 36298 | WINMPHDRD |
| 36299 | WINMPHDRL |
| 36300 | WINMPHDRN |
| 36301 | WINMPHDRP |
| 36302 | WINMPHDRR |
| 36303 | WINMPHDRY |
| 36304 | WINMPHGRH |
| 36305 | WINMPLDRH |
| 36306 | WINMPNDRH |
| 36307 | WINMPPDRH |
| 36308 | WINMPRDRH |
| 36309 | WINMTHDRH |
| 36310 | WINRPHDRH |
| 36311 | WINVPHDRH |
| 36312 | WIPAKTYWC |
| 36313 | WIPAMAYWC |
| 36314 | WIPAMPYWC |
| 36315 | WIPAMTYLC |
| 36316 | WIPAMTYWF |
| 36317 | WIPAMTYWW |
| 36318 | WIPAMTYWY |
| 36319 | WIPARTYWC |
| 36320 | WIPAVTYWC |
| 36321 | WIPGMTYWC |
| 36322 | WIPSMTYWC |
| 36323 | WIPTMTYWC |
| 36324 | WIPVVTYWC |
| 36325 | WISAMTYWC |
| 36326 | WISMPHDRH |
| 36327 | WITAMTYWC |
| 36328 | WITMPHDRH |
| 36329 | WKNVCPSRQ |
| 36330 | WKSVCPSRQ |
| 36331 | WKTVCPSRH |
| 36332 | WLCQMWSWW |
| 36333 | WLPAMTYWC |
| 36334 | WMLLGLTWF |
| 36335 | WMLLGMAWC |
| 36336 | WMLLGMHWC |
| 36337 | WMLLGMNWC |
| 36338 | WMLLGMPWC |
| 36339 | WMLLGMTLC |
| 36340 | WMLLGMTWF |
| 36341 | WMLLGMTWG |
| 36342 | WMLLGMTWW |
| 36343 | WMLLGMTWY |
| 36344 | WMLLGTTWC |
| 36345 | WMLLGVTWC |
| 36346 | WMLLVMTWC |
| 36347 | WMLNQSRGW |
| 36348 | WMLQGMTWC |
| 36349 | WMMLGMTWC |
| 36350 | WMMMGLTWC |
| 36351 | WMNMPHDRH |
| 36352 | WMPNQSRVW |
| 36353 | WMVLGMNWC |
| 36354 | WMVLGMTWC |
| 36355 | WNCSVYTCI |
| 36356 | WNNWSNTQI |
| 36357 | WRLLGMTWC |
| 36358 | WSCKMWSWW |
| 36359 | WTLLGMTWC |
| 36360 | WVCKMWGWW |
| 36361 | WVCKMWSWW |
| 36362 | WVNLCADLI |
| 36363 | WVPAMTYWC |
| 36364 | WVPAPWRTH |
| 36365 | WVPPPWRTH |
| 36366 | WVPTHWRTH |
| 36367 | WVPTPGRTH |
| 36368 | WVPTPWQTH |
| 36369 | WVPTPWRSH |
| 36370 | WVPTPWRTN |
| 36371 | WVPTPWRTQ |
| 36372 | WVPTPWRTY |
| 36373 | WVPTTWRTH |
| 36374 | WVTLCAVLI |
| 36375 | WVTTPWRTH |
| 36376 | WYMSKQYED |
| 36377 | WYMSQEYED |
| 36378 | WYMSQQNED |
| 36379 | WYMSQQSED |
| 36380 | WYMSQQYAD |
| 36381 | WYMSQQYKD |
| 36382 | WYMSQQYQD |
| 36383 | WYMSQRYED |
| 36384 | WYMTQQYED |
| 36385 | WYTSQQYED |
| 36386 | WYVKFWETM |
| 36387 | WYVSQQYED |
| 36388 | YAQPDWFMT |
| 36389 | YAQRDWFMA |
| 36390 | YAWFYWADY |
| 36391 | YDQPDWFMA |
| 36392 | YDYWPMCWS |
| 36393 | YFMEWEVMD |
| 36394 | YFWYSMAFP |
| 36395 | YHYWHMCWS |
| 36396 | YHYWPMCWA |
| 36397 | YHYWPMCWC |
| 36398 | YHYWPMCWP |
| 36399 | YHYWPMCWT |
| 36400 | YHYWPMCWY |
| 36401 | YHYWPMGWS |
| 36402 | YIIRCMLIW |
| 36403 | YKNWYINMH |
| 36404 | YLEAKTIHA |
| 36405 | YLESKTIHP |
| 36406 | YPELGKGEI |
| 36407 | YPELGQCEI |
| 36408 | YPQPDWFMA |
| 36409 | YPYWPMCWS |
| 36410 | YSWFNWADY |
| 36411 | YSWFYWADC |
| 36412 | YSWFYWADF |
| 36413 | YSWFYWADS |
| 36414 | YSWFYWAVY |
| 36415 | YSWFYWPDY |
| 36416 | YSWFYWSDY |
| 36417 | YSWFYWTDY |
| 36418 | YSWFYWVDY |
| 36419 | YSWIYWADY |
| 36420 | YSWVYWADY |
| 36421 | YSWYYWADY |
| 36422 | YTALMWVWA |
| 36423 | YTELGKCEI |
| 36424 | YTLLWYGIC |
| 36425 | YTNFEAVLI |
| 36426 | YTNFEDALI |
| 36427 | YTNFEDVII |
| 36428 | YTNFEGVLI |
| 36429 | YTNFENVLI |
| 36430 | YTNIEDVLI |
| 36431 | YTNLDDVLI |
| 36432 | YTWFYWADY |
| 36433 | YWSHIIGIY |
| 36434 | YWSNIIGIY |
| 36435 | YYAQYYRTW |
| 36436 | YYWFYWADY |
| 36437 | YYWFYWSDY |

### Example 42

AAV5 Variants with Spleen Tissue Tropism as Identified by Machine Learning

This example describes preferred properties of engineered AAV5 variants that display spleen tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display spleen tissue tropism.

Favored biophysical properties and favored amino acid residues at each position in the 581 to 589 region of an AAV5 VP capsid polypeptide, which are associated with a higher probability of spleen tissue tropism are described below. Any of the below machine learning-derived positional amino acid preferences described in TABLE 68 can be present alone or in combination with each other or in combination with any of the amino acid preferences observed and described in EXAMPLE 6, TABLE 11. Listed below in TABLE 68 are a summary of positional features shared between the top important features for spleen tropism extracted from the ML models.

TABLE 68
Machine Learning-Derived Spleen Tissue Tropism Rules

Low solubility at Xaa1
Xaa1 is selected from D or P
Or
High solubility
Xaa1 is selected from F, I, or L
Low hydropathy at Xaa1
Xaa1 is selected from Y or P
Low mutability at Xaa1
Xaa1 is selected from C, K, or P
Low solubility at Xaa2
Xaa2 is selected from D, Q, or R
Low hydropathy at Xaa2
Xaa2 is selected from D, E, R, K, H, N, or Q
Low charge at Xaa2
Xaa2 is selected from D or E
Low volume at Xaa2
Xaa2 is selected from T, N, P, or D
High average flexibility at Xaa2
Xaa2 is selected from D, E, R, P, G, Q, or S
Low solubility at Xaa3
Xaa3 is selected from D, E, P, or N
Low hydropathy at Xaa3
Xaa3 is selected from D, E, H, N, Q, or P
Low hydropathy at Xaa4
Xaa4 is selected from K or R
Low solubility at Xaa5
Xaa5 is selected from D, E, P, or N
High average flexibility at Xaa5
Xaa5 is selected from D, E, R, P, G, Q, or S
Low mutability at Xaa6
Xaa6 is selected from C
High surface accessibility at Xaa8
Xaa8 is selected from E, R, or K
Low solubility at Xaa8
Xaa8 is selected from E, P, R, K, N, or Q
Medium volume at Xaa8
Xaa8 is selected from E, D, R, K, V, P, M, I, L, H, N, Q, or T
Medium mol mass at Xaa9
Xaa9 is selected from E, D, K, M, I, L, H, or N

TABLE 69 below provides sequences of the 581 to 589 region for variant AAV VP1 capsid polypeptides that exhibited spleen tissue tropism and comport to one or more of the rules provided in TABLE 68. The present disclosure, thus, provides for rAAVs composed of engineered AAV5 VP1 capsid polypeptides having a SEQ ID NO: 2, wherein the Xaa1 to Xaa9 region is any one of SEQ ID NO: 38438-SEQ ID NO: 39437, as disclosed in TABLE 69. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 69
Sequences of the 581 to 589 Region in AAV5 VP1
Capsid Polypeptide that Drive Spleen Tissue
Tropism

| SEQ | |
|---|---|
| ID | 581-589 |
| NO | Sequence |
| 38438 | ADDMCCSPN |
| 38439 | ADEDDCDDP |
| 38440 | ADEMEEEHG |
| 38441 | ADEMWPIRF |
| 38442 | ADHCNWSRT |
| 38443 | ADHCSWSRT |
| 38444 | ADMCECVKG |
| 38445 | ADNRTKPLC |
| 38446 | ADPDQKAMN |
| 38447 | ADQRDCWTT |
| 38448 | ADREPWPRN |
| 38449 | ADSRVRPKW |
| 38450 | AECKCCVKM |
| 38451 | AENQDDWQF |
| 38452 | AGDMWRHKM |
| 38453 | AGDTDMPED |
| 38454 | AGWEEAWCM |
| 38455 | AHDCDEPAL |
| 38456 | AIENNYCPM |
| 38457 | AIPHWDDKH |
| 38458 | APDFNREKN |
| 38459 | APDHWMEKM |
| 38460 | ARKCDFPAL |
| 38461 | CDAKRTPGM |
| 38462 | CDAWGELRG |
| 38463 | CDCKEQEED |
| 38464 | CDCKGRIPH |
| 38465 | CDDFCMCHC |
| 38466 | CDDHNHPRA |
| 38467 | CDDKQSHKE |
| 38468 | CDDKRFSKK |
| 38469 | CDDRERQQN |
| 38470 | CDDWGWDQM |
| 38471 | CDEIEMQGT |
| 38472 | CDELPMAGH |
| 38473 | CDEPHFWRC |
| 38474 | CDERRWDRP |
| 38475 | CDIRCENMG |
| 38476 | CDMCREWKW |
| 38477 | CDNKCWPHD |
| 38478 | CDNKPKNLS |
| 38479 | CDNRRNWWE |
| 38480 | CDPCNEFNK |
| 38481 | CDPMDACQE |
| 38482 | CDPNDMREH |
| 38483 | CDPRFVEAM |
| 38484 | CDPRTLPFY |
| 38485 | CDPWDWDIE |
| 38486 | CDPWDWEIE |
| 38487 | CDQFCEWRQ |
| 38488 | CDQFHWDKK |
| 38489 | CDRRCFDKH |
| 38490 | CDSCCGMHV |
| 38491 | CDTWPWSKK |
| 38492 | CDWCDQFRH |
| 38493 | CDYCDPRKF |
| 38494 | CDYRCLWYC |
| 38495 | CECYEPFEG |
| 38496 | CEDMDQWPW |
| 38497 | CELPDRCPQ |
| 38498 | CERVPVFSF |
| 38499 | CFDHRPLPC |
| 38500 | CFPCCWAKF |
| 38501 | CGAWWWNHM |
| 38502 | CGDEWAPRL |
| 38503 | CGDIDHPKS |
| 38504 | CGDWAWDRC |
| 38505 | CGEKCWHDI |
| 38506 | CGHDSKPRC |
| 38507 | CGHWCCSDY |
| 38508 | CGIEDKPEN |
| 38509 | CGLCRYYDM |
| 38510 | CGPCCLWGM |
| 38511 | CGPEPCVRQ |
| 38512 | CGPWNEPEY |
| 38513 | CGTHCPLRQ |
| 38514 | CGYDCQPRF |
| 38515 | CHKFDRELW |
| 38516 | CHNHPMPPL |
| 38517 | CKDEFWPLM |
| 38518 | CKDFRMSPL |
| 38519 | CKDYCRDEL |
| 38520 | CKPYEWWVM |
| 38521 | CKYTDDEEQ |
| 38522 | CMTDWSGMM |
| 38523 | CNCKDYVEV |
| 38524 | CNDQHMEKD |
| 38525 | CNPPEEPVR |
| 38526 | CNTQPSPKD |
| 38527 | CPEYRMARF |
| 38528 | CPPPCSYLL |
| 38529 | CPQWCEYYE |
| 38530 | CQCDDNWEI |
| 38531 | CQERERGKW |
| 38532 | CQSECAKRG |
| 38533 | CRDCRDGEN |
| 38534 | CRDEGLEYE |
| 38535 | CRDGDEPRG |
| 38536 | CRDWGWICA |
| 38537 | CRNDNIERK |
| 38538 | CRPLDWKPD |
| 38539 | CRYWCSYKD |
| 38540 | CWDGCHRRL |
| 38541 | CWDHGAPEL |
| 38542 | CWEDCQYHE |
| 38543 | CYDDWQDRI |
| 38544 | DCDMFCMFN |
| 38545 | DDARRCHRK |
| 38546 | DDCFQRCRN |
| 38547 | DDDCNYTRN |
| 38548 | DDDEDLNYD |
| 38549 | DDDHPRPRS |
| 38550 | DDDNWHHKN |
| 38551 | DDDPGDAYW |
| 38552 | DDECEMYYN |
| 38553 | DDEDGGHYM |
| 38554 | DDEPKGIRM |
| 38555 | DDEVDGDHP |
| 38556 | DDGCNYTRN |
| 38557 | DDGPDTNRK |
| 38558 | DDGPDTYRR |
| 38559 | DDGPETYRK |
| 38560 | DDGVMDSVD |
| 38561 | DDHEEGFKA |
| 38562 | DDHQENTKT |
| 38563 | DDKGPRNSM |
| 38564 | DDKHDRIHH |
| 38565 | DDLKCWNNY |
| 38566 | DDMHDNMVH |
| 38567 | DDNCNYTRN |
| 38568 | DDNHPTPTL |
| 38569 | DDNREPQDT |
| 38570 | DDNWDVFRD |
| 38571 | DDPCVEFET |
| 38572 | DDPFDVTLQ |
| 38573 | DDPFWCQRL |
| 38574 | DDPQEVHGF |
| 38575 | DDPQRKGYC |
| 38576 | DDPWGDRHT |
| 38577 | DDQLEGWID |
| 38578 | DDQPDNNIY |
| 38579 | DDQRLLDDI |
| 38580 | DDQTDYPPT |
| 38581 | DDQYRIEQH |
| 38582 | DDRANVPKH |
| 38583 | DDSGPKSQI |
| 38584 | DDSIEHFKG |
| 38585 | DDSWPIPLG |
| 38586 | DDTEPMMNK |
| 38587 | DDTEPMPVR |
| 38588 | DDTNEIEEY |
| 38589 | DDWKGWLRF |
| 38590 | DDWKPCEFA |
| 38591 | DDWMRPKRC |
| 38592 | DDYGHDDKN |
| 38593 | DECSESMRN |
| 38594 | DEGEEEPKL |
| 38595 | DEHKDPDEA |
| 38596 | DENKDEFWM |
| 38597 | DEQCRTHRK |
| 38598 | DEQHGGEMD |
| 38599 | DESCEWMNQ |
| 38600 | DESEERIQD |
| 38601 | DESSDDDEE |
| 38602 | DGCFDAQIN |
| 38603 | DGDHDARIH |
| 38604 | DGETDELRM |
| 38605 | DGFLDGEWW |
| 38606 | DGFMDGEWC |
| 38607 | DGKTSREVC |
| 38608 | DGPKDRIFN |
| 38609 | DGPVPWMMC |
| 38610 | DGSDGMSTN |
| 38611 | DGVFPRCGN |
| 38612 | DHCKWWLDV |
| 38613 | DHDCKCEEA |
| 38614 | DHDGDVPVF |
| 38615 | DHHCDRPQG |
| 38616 | DHQCEFPMV |
| 38617 | DHTCNWPPN |
| 38618 | DICFRVSMM |
| 38619 | DIEDTQMSW |
| 38620 | DIEQWIAAM |
| 38621 | DKDFWSAPN |
| 38622 | DKHLEWPNH |
| 38623 | DKKNECNWH |
| 38624 | DKTCDCEMT |
| 38625 | DLSDQYPEL |
| 38626 | DLWCMDAEW |
| 38627 | DLWTHWNPC |
| 38628 | DMDEGSSSN |
| 38629 | DMDKDGPRE |
| 38630 | DMWDWTYRM |
| 38631 | DNDTWQPRI |
| 38632 | DNEWDCAGN |
| 38633 | DNHREAWQD |
| 38634 | DNKRRVPEC |
| 38635 | DNQCEDPKP |
| 38636 | DPEMEYMGM |
| 38637 | DPKPRHDND |
| 38638 | DQDICGWVW |
| 38639 | DQDRRCDQP |
| 38640 | DQECDDHGI |
| 38641 | DQHGWRRPG |
| 38642 | DQKCERGRI |
| 38643 | DQNHRVDRH |
| 38644 | DQRHDHRQM |
| 38645 | DQSSGWPRD |
| 38646 | DQWNDECFF |
| 38647 | DRERWWPET |
| 38648 | DRKFERQTM |
| 38649 | DRKKPVPEW |
| 38650 | DRNQWLPRH |
| 38651 | DRPRPYCRN |
| 38652 | DRQYPPPNH |
| 38653 | DRSPWCPQF |
| 38654 | DRSQRCNRG |
| 38655 | DRTGRCMPP |
| 38656 | DRYLGWQFC |
| 38657 | DVDCCCVYN |
| 38658 | DWDCKTSRN |
| 38659 | DWKPADNKC |
| 38660 | DWNSDWPEY |
| 38661 | DWQRHPPKC |
| 38662 | DWRPPCFRF |
| 38663 | DWSKEIMGM |
| 38664 | DWTCNGHRH |
| 38665 | DYDWSDMKC |
| 38666 | DYENDEDGV |
| 38667 | DYKCCRPEY |
| 38668 | EDDFKPARD |
| 38669 | EDEGDLWFY |
| 38670 | EDEPDGKMG |
| 38671 | EDEQSGERE |
| 38672 | EDGHKVGFM |
| 38673 | EDHDIPRRN |
| 38674 | EDNRWIPPL |
| 38675 | EDPGDPDMM |
| 38676 | EDPHEEFDR |
| 38677 | EDQCCFELP |
| 38678 | EDTQPLFRH |
| 38679 | EEAHRGERC |
| 38680 | EEDHEQMCN |
| 38681 | EFDHWTMRM |
| 38682 | EFEHELDIW |
| 38683 | EFPVPLDFW |
| 38684 | EGAEGWTQD |
| 38685 | EGDPPMPVF |
| 38686 | EHDCQDDVN |
| 38687 | EHEWDAPSW |
| 38688 | EHKYRDMKH |
| 38689 | EHPMELEFC |
| 38690 | EIDFGTPET |
| 38691 | EIDYWWCPH |
| 38692 | EMDLCHYSN |
| 38693 | EMHPCFWIF |
| 38694 | EPSRCRPHV |
| 38695 | EQKQEPEFW |
| 38696 | ERCEGIQFC |
| 38697 | ERDAGDTIN |
| 38698 | ERHGEEIRD |
| 38699 | ERHYDIMLY |
| 38700 | ERKFYENRM |
| 38701 | ERQKDRLPD |
| 38702 | ESRYCMWGM |
| 38703 | ETRELITMM |
| 38704 | EVDEDPAFW |
| 38705 | EVDFCSAIM |
| 38706 | EVEHDVAVW |
| 38707 | EVTMGCMIN |
| 38708 | FDCDDVIER |
| 38709 | FDDCDAGRG |
| 38710 | FDDDDKAKC |
| 38711 | FDDYDLTVE |
| 38712 | FDETDFEAM |
| 38713 | FDFPDCHGW |
| 38714 | FDHDDEHKC |
| 38715 | FDHHEKWNN |
| 38716 | FDHRPNCHD |
| 38717 | FDNPDQRNA |
| 38718 | FDNRDSDKG |
| 38719 | FDNRRHWWE |
| 38720 | FDPFQFCVG |
| 38721 | FDPHHAKQQ |
| 38722 | FDPIYEMVE |
| 38723 | FDSFDEHHN |
| 38724 | FDWISKCSW |
| 38725 | FDWQWEDKC |
| 38726 | FDYKWMNAY |
| 38727 | FEDWGWPKG |
| 38728 | FEHRDNWKM |
| 38729 | FERWDRKEH |
| 38730 | FERYDKPYM |
| 38731 | FGEMDGGCY |
| 38732 | FGGCDGTPW |
| 38733 | FGWLDCCNQ |
| 38734 | FGWPDEQQW |
| 38735 | FHETQTPKT |
| 38736 | FKACDSECD |
| 38737 | FKCGNWGII |
| 38738 | FKDYRCGWC |
| 38739 | FKEGDAKKD |
| 38740 | FKGCSGELV |
| 38741 | FKMCPCVKG |
| 38742 | FKMSDILPH |
| 38743 | FKPCMCDPM |
| 38744 | FKPDPCFDD |
| 38745 | FKPTNIEKG |
| 38746 | FKWWDTMNQ |
| 38747 | FKYTDHGIQ |
| 38748 | FLNDWSNWF |
| 38749 | FNPFDCDIG |
| 38750 | FNTFDCEIG |
| 38751 | FRCNERRWN |
| 38752 | FRCYPNQRD |
| 38753 | FRDRDGNRM |
| 38754 | FRGGEMAKN |
| 38755 | FRNENEFEP |
| 38756 | FRPKCGPIH |
| 38757 | FRPKECALQ |
| 38758 | FRQCGEEYH |
| 38759 | FRQLDRERQ |
| 38760 | FRQWQTMET |
| 38761 | FRSEEKAEN |
| 38762 | FRSRNPPKH |
| 38763 | FRSYGIRLI |
| 38764 | FRTWPDDET |
| 38765 | FWEHADEEG |
| 38766 | FYDGPREMW |
| 38767 | FYVGPAMPM |
| 38768 | GADWDEPKE |
| 38769 | GDCIDGKPH |
| 38770 | GDDHDIEFM |
| 38771 | GDDHKSHRK |
| 38772 | GDDLDCWKN |
| 38773 | GDEDHGSKM |
| 38774 | GDELHFLRM |
| 38775 | GDERPGRRN |
| 38776 | GDLCRYWRD |
| 38777 | GDPDDDEIW |
| 38778 | GEAIDNEKW |
| 38779 | GEEEDRMIH |
| 38780 | GESCDEKSD |
| 38781 | GIPCWQDEW |
| 38782 | GKPEDGFDD |
| 38783 | GPMRRRINI |
| 38784 | GRCQPWYFH |
| 38785 | GRDCDEAPD |
| 38786 | GRDFYWKIE |
| 38787 | GRECSAQRM |
| 38788 | GRHGDDLEV |
| 38789 | GRPQCWYKH |
| 38790 | GRTCDYHDM |
| 38791 | HDATPTPRY |
| 38792 | HDCCEWWRA |
| 38793 | HDCWAEWIA |
| 38794 | HDDGDGFQF |
| 38795 | HDDIEAIRQ |
| 38796 | HDEGPEFKD |
| 38797 | HDELWVDRM |
| 38798 | HDESGEWLE |
| 38799 | HDHKWKPPE |
| 38800 | HDKWENPGQ |
| 38801 | HDLQPGMNN |
| 38802 | HDPCTQIKP |
| 38803 | HDPWNVLDW |
| 38804 | HDYEPKCMG |
| 38805 | HEFKDDMPH |
| 38806 | HENKEEQKD |
| 38807 | HEQWDNIPC |
| 38808 | HFECRTMPN |
| 38809 | HFEFWNGGM |
| 38810 | HGDHRGNRE |
| 38811 | HGSMDSVYW |
| 38812 | HHQHNEIEL |
| 38813 | HKECPRQRH |
| 38814 | HKGFEPERQ |
| 38815 | HKPCHGTEF |
| 38816 | HMEDWMLEM |
| 38817 | HNDTWQPRL |
| 38818 | HNEGERWPN |
| 38819 | HQCMWDWLT |
| 38820 | HRCCETTIN |
| 38821 | HRCPIDSRI |
| 38822 | HRECGVWDF |
| 38823 | HRSDDDQRF |
| 38824 | HRSDPPQKM |
| 38825 | HTCRLDVNM |
| 38826 | HVEEWAEKM |
| 38827 | HWSIDAPRK |
| 38828 | IDAKPYGRE |
| 38829 | IDAWEEHYE |
| 38830 | IDCCSFSPT |
| 38831 | IDCQRWCQQ |
| 38832 | IDCTPWIQT |
| 38833 | IDDDPEPTR |
| 38834 | IDDKKWIHA |
| 38835 | IDDRIWNCD |
| 38836 | IDDWLMDKP |
| 38837 | IDEWDCIDS |
| 38838 | IDFHDCFTK |
| 38839 | IDFNDWIPW |
| 38840 | IDGDDEHRA |
| 38841 | IDHKEAFFE |
| 38842 | IDIHEREHF |
| 38843 | IDKWEWNRD |
| 38844 | IDLQDCNPW |
| 38845 | IDMTDMVLW |
| 38846 | IDPEGESIS |
| 38847 | IDPFDWEGM |
| 38848 | IDPFWTFKF |
| 38849 | IDPPEQDIM |
| 38850 | IDPYDCDPT |
| 38851 | IECNDDCPE |
| 38852 | IEKFDDPPA |
| 38853 | IEPWEHMSN |
| 38854 | IEQKGWRRD |
| 38855 | IESDPCVAG |
| 38856 | IEWDPWIHL |
| 38857 | IEWFRWMMW |
| 38858 | IFDPRVKAW |
| 38859 | IGENMWIWH |
| 38860 | IHPMRIGPW |
| 38861 | IHTVPDWVM |
| 38862 | IIVEVEICH |
| 38863 | IKDGPEIDP |
| 38864 | IKFWDGLRD |
| 38865 | IKPCDEPVD |
| 38866 | IKPIGFWVN |
| 38867 | IKVKPDNIM |
| 38868 | IKWPDEHIW |
| 38869 | INPYGTPLN |
| 38870 | IPWCDYFFA |
| 38871 | IQDCNSIRY |
| 38872 | IQERMSIED |
| 38873 | IQLGEFWKE |
| 38874 | IQWMDMDPW |
| 38875 | IRDFGERRI |
| 38876 | IRDIDYLPW |
| 38877 | IRDMSCLPS |
| 38878 | IRGDEEDRN |
| 38879 | IRGDEMLED |
| 38880 | IRGREDMLQ |
| 38881 | IRHCDGDCE |
| 38882 | IRHEDGCTY |
| 38883 | IRPCAPARR |
| 38884 | IRPDGEEIQ |
| 38885 | IRPNSSLEL |
| 38886 | IRPTGTTPN |
| 38887 | IRSCPMKCP |
| 38888 | ITCDWYYQM |
| 38889 | IVSPADIVP |
| 38890 | IWERDPDGE |
| 38891 | IYCMPYIRA |
| 38892 | KDCNHRAEN |
| 38893 | KDDCAAWNC |
| 38894 | KDEDDIWID |
| 38895 | KDHGHQLPP |
| 38896 | KDHLGGKRN |
| 38897 | KDHMGCPPH |
| 38898 | KDHWHPPKY |
| 38899 | KDKMKWLKE |
| 38900 | KDMCYLSKK |
| 38901 | KDNFWMYKT |
| 38902 | KDNGNKMDE |
| 38903 | KDRTQIWKQ |
| 38904 | KDSPWFERT |
| 38905 | KDSQRHDKE |
| 38906 | KDVFWENKF |
| 38907 | KDWSELYEY |
| 38908 | KDYIPQLRG |
| 38909 | KDYNDQEEN |
| 38910 | KEKIQDYPE |
| 38911 | KEWFDWCIT |
| 38912 | KPLKCWEPQ |
| 38913 | KRDYWCWPM |
| 38914 | LCSFDDSII |
| 38915 | LCSFGDSIS |
| 38916 | LDCMRWGFN |
| 38917 | LDCPPAPKM |
| 38918 | LDCRPEHPD |
| 38919 | LDDEESLVF |
| 38920 | LDDWDFKPW |
| 38921 | LDECEIECN |
| 38922 | LDEFKMEPQ |
| 38923 | LDGFDTNLE |
| 38924 | LDGTDAVRL |
| 38925 | LDHKWPDYI |
| 38926 | LDIRWYEHV |
| 38927 | LDLWRNWKI |
| 38928 | LDPCAWVKV |
| 38929 | LDPFAQPFS |
| 38930 | LDPTDPGRW |
| 38931 | LDRLEAPTG |
| 38932 | LDRNRVHRQ |
| 38933 | LDSCDPRLK |
| 38934 | LDTCEMLKQ |
| 38935 | LDTGEMLKQ |
| 38936 | LDTKRWPKY |
| 38937 | LDWKDKERG |
| 38938 | LDYRDIARD |
| 38939 | LEAGEKWNF |
| 38940 | LEAHEGEKF |
| 38941 | LEDLRLPRM |
| 38942 | LENKWCYKM |
| 38943 | LENRPELND |
| 38944 | LESCSPIVS |
| 38945 | LEWIHDWYH |
| 38946 | LEWYECVVT |
| 38947 | LGDCPGEPH |
| 38948 | LGLCDREMQ |
| 38949 | LGSFDDSIS |
| 38950 | LHDWWMREM |
| 38951 | LHQRDRIEF |
| 38952 | LKCGSLIDV |
| 38953 | LKLADWWTT |
| 38954 | LKLRDEVNE |
| 38955 | LKMERCMIA |
| 38956 | LKPFEDLLN |
| 38957 | LKQWDPWLC |
| 38958 | LLDCRGPEV |
| 38959 | LNDHEMPGM |
| 38960 | LNRCDAPKL |
| 38961 | LNWPDYHCM |
| 38962 | LQCREGLPM |
| 38963 | LQDEPEPPH |
| 38964 | LQDFRFCKQ |
| 38965 | LQDPACDPS |
| 38966 | LQHRDSVES |
| 38967 | LRCKWWRTV |
| 38968 | LRDFWECRL |
| 38969 | LRDWDGNRM |
| 38970 | LREVDYPSM |
| 38971 | LREWKWCPD |
| 38972 | LRPCEGGQD |
| 38973 | LRPDIAWEC |
| 38974 | LRPGGGLKK |
| 38975 | LRTIWSPRM |
| 38976 | LRTNDCNFH |
| 38977 | LRVMDPLEG |
| 38978 | LRYNDLKIH |
| 38979 | LTGCSDEVQ |
| 38980 | LWADGWELK |
| 38981 | LYQCTDPEK |
| 38982 | LYQPGEPKS |
| 38983 | MDACEGQNN |
| 38984 | MDARDECYQ |
| 38985 | MDCRREHRH |
| 38986 | MDDFEAIRW |
| 38987 | MDDSEVLKY |
| 38988 | MDEEFWPFN |
| 38989 | MDERDAHEE |
| 38990 | MDERWWIKI |
| 38991 | MDFEEMWVW |
| 38992 | MDHKDGYRQ |
| 38993 | MDHLHKPKT |
| 38994 | MDNHKKPRA |
| 38995 | MDPDPKCGM |
| 38996 | MDPSHGKIN |
| 38997 | MEGFDGWRM |
| 38998 | MEMYRWCRH |
| 38999 | MFDCRFPRY |
| 39000 | MFDFWNLTM |
| 39001 | MGECDWPMH |
| 39002 | MGMEDDWKH |
| 39003 | MHPWDALDM |
| 39004 | MHYFRYCKN |
| 39005 | MKDFWTEKW |
| 39006 | MMNHHDTRP |
| 39007 | MNPMAYTRN |
| 39008 | MQPFQMHRG |
| 39009 | MRCDGWIQF |
| 39010 | MRCDLKKKE |
| 39011 | MRDWEASKM |
| 39012 | MRQFGCPTE |
| 39013 | NDCMHNDRW |
| 39014 | NDDEPGWKQ |
| 39015 | NDDEPPSRL |
| 39016 | NDDFDCQEA |
| 39017 | NDDLDCCVK |
| 39018 | NDDRCFIRA |
| 39019 | NDEIDAQYH |
| 39020 | NDEIECIGH |
| 39021 | NDMFPSFDC |
| 39022 | NDPGNRPRE |
| 39023 | NDPRRPDSN |
| 39024 | NDRCEKPDI |
| 39025 | NDRPEADEH |
| 39026 | NDSMGEPKS |
| 39027 | NDSYGQPKV |
| 39028 | NDTGPHGRH |
| 39029 | NDTNRALKM |
| 39030 | NDYIDEFPY |
| 39031 | NDYVEEHPM |
| 39032 | NECCDRHPM |
| 39033 | NEDHENPSN |
| 39034 | NEDQEEDPC |
| 39035 | NEYRPPPQV |
| 39036 | NGYFDAMRH |
| 39037 | NHCDENWYW |
| 39038 | NHCKDWEEH |
| 39039 | NKPIWYVEM |
| 39040 | NPENHKCRF |
| 39041 | NQEKTEPKC |
| 39042 | NQEMEYHPS |
| 39043 | NQTCDQEEM |
| 39044 | NRDWFEWKM |
| 39045 | NRDWRFFRI |
| 39046 | NREQDELKW |
| 39047 | NREWPDYRD |
| 39048 | NRGVDRLKW |
| 39049 | NRPEGRGEC |
| 39050 | NRVLEIERK |
| 39051 | NRYFDAMRH |
| 39052 | NTDFECVGN |
| 39053 | NVCEQDIYE |
| 39054 | PDALDPCDP |
| 39055 | PDAYDDRNH |
| 39056 | PDCEPGNVE |
| 39057 | PDDCPWPNN |
| 39058 | PDDFDKFMW |
| 39059 | PDDFEGGPL |
| 39060 | PDDHESPRD |
| 39061 | PDDHETPTN |
| 39062 | PDDIHRFPK |
| 39063 | PDEQSLPFD |
| 39064 | PDEVPRCRA |
| 39065 | PDKLDRFVG |
| 39066 | PDLFEKCRI |
| 39067 | PDLRDHWQM |
| 39068 | PDNAQGPQC |
| 39069 | PDNEDARPK |
| 39070 | PDPADKCTY |
| 39071 | PDPEGEDEP |
| 39072 | PDPGIRWPM |
| 39073 | PDPHEDGNP |
| 39074 | PDPLGWLAH |
| 39075 | PDPPKKYED |
| 39076 | PDPPKQYED |
| 39077 | PDPQQMSRR |
| 39078 | PDPRYWIMN |
| 39079 | PDPSDKGTY |
| 39080 | PDPSPAKGV |
| 39081 | PDPSPYCKS |
| 39082 | PDPWPWMDD |
| 39083 | PDQDDMMHG |
| 39084 | PDQFENSFK |
| 39085 | PDQFIRWPM |
| 39086 | PDQHDPSNL |
| 39087 | PDQSHIDLM |
| 39088 | PDRCDLTDF |
| 39089 | PDRCNYVRH |
| 39090 | PDSGEQPVA |
| 39091 | PDSPGVCRC |
| 39092 | PDTMWREHG |
| 39093 | PDTYHYCEW |
| 39094 | PDVNQGPWL |
| 39095 | PDVSERMMD |
| 39096 | PECGDRGDR |
| 39097 | PEDCPDGKA |
| 39098 | PEDEWGFEY |
| 39099 | PEEDEHDVC |
| 39100 | PEEDSPWIM |
| 39101 | PEFGWWIAM |
| 39102 | PEHHFRWPH |
| 39103 | PEHKDHFET |
| 39104 | PEKLDLINY |
| 39105 | PEKYDIWPV |
| 39106 | PEMEDASLG |
| 39107 | PENDHGMEA |
| 39108 | PESFPAERC |
| 39109 | PESNDKRES |
| 39110 | PETGKAPHI |
| 39111 | PETIRGGKC |
| 39112 | PEVNEGIHL |
| 39113 | PEYPKKGKC |
| 39114 | PEYYDDDHS |
| 39115 | PFPRDWHDA |
| 39116 | PGAIDWQIM |
| 39117 | PGCCDRLVE |
| 39118 | PGDFGTAED |
| 39119 | PGGMCWWER |
| 39120 | PGGNDCLLF |
| 39121 | PGPKPWKEI |
| 39122 | PGPNDVNHC |
| 39123 | PGSHGPPVC |
| 39124 | PGTMDPPVN |
| 39125 | PGYNRLEPS |
| 39126 | PHCEEGPED |
| 39127 | PHDFWVIED |
| 39128 | PHDNDAYFW |
| 39129 | PHDRRKNDN |
| 39130 | PHEIEYYMN |
| 39131 | PHEWDMCAE |
| 39132 | PHNNHWIEP |
| 39133 | PHNQWMPKS |
| 39134 | PHRHGYRKD |
| 39135 | PHRQDRMIW |
| 39136 | PHTCCGPEE |
| 39137 | PHTDRLDIN |
| 39138 | PHWFNWPMC |
| 39139 | PHYNTVPEN |
| 39140 | PKCSNWLNM |
| 39141 | PKCTDWNGV |
| 39142 | PKDNPIEPK |
| 39143 | PKDTKGPET |
| 39144 | PKEQPCAEY |
| 39145 | PKEWDKPPH |
| 39146 | PKHEDCCPE |
| 39147 | PKKYDRDVM |
| 39148 | PKPWETSRC |
| 39149 | PKQGPAIFN |
| 39150 | PKSCGDGND |
| 39151 | PKTCNCKVT |
| 39152 | PKYHESAKS |
| 39153 | PLQDGCEMH |
| 39154 | PMHYWKSEM |
| 39155 | PMPLCCWSL |
| 39156 | PNALDMCVE |
| 39157 | PNEPWCPVV |
| 39158 | PNESNEIEH |
| 39159 | PNNAWIDKQ |
| 39160 | PNNEENDAM |
| 39161 | PNNNDQDKT |
| 39162 | PNPHDRPRW |
| 39163 | PNQLDKPAM |
| 39164 | PNQRNEIEH |
| 39165 | PNWGIEPGF |
| 39166 | PQCKPLVPM |
| 39167 | PQGCDKYLW |
| 39168 | PQHYDKWEI |
| 39169 | PQLPELEKW |
| 39170 | PQNGHICKG |
| 39171 | PQNYDQDKV |
| 39172 | PQPNRTSPH |
| 39173 | PQSEPAPYD |
| 39174 | PQSFWGSEM |
| 39175 | PRCDPCMGV |
| 39176 | PREGDWEGM |
| 39177 | PREMWECQC |
| 39178 | PREQRPSKS |
| 39179 | PRGLPTPMN |
| 39180 | PRNCPEDWS |
| 39181 | PRNYEAAEC |
| 39182 | PRRMPLVPH |
| 39183 | PRSDDDQRF |
| 39184 | PRSGDDQRF |
| 39185 | PRTDGCPWA |
| 39186 | PRYQDRCMW |
| 39187 | PTPDGSDKN |
| 39188 | PTVPQCPRH |
| 39189 | PVPHSCERR |
| 39190 | PWFHKRYKN |
| 39191 | PWNISCPKE |
| 39192 | PWPKWCWPA |
| 39193 | PWQFGHAKQ |
| 39194 | PWTYRGEDF |
| 39195 | PYDQRTDDA |
| 39196 | PYGPMCTMG |
| 39197 | PYKMNHPRE |
| 39198 | PYNCRRCPD |
| 39199 | PYNPNVDPH |
| 39200 | QDAMDPWRI |
| 39201 | QDCVERIKS |
| 39202 | QDEIDPSEH |
| 39203 | QDEIRMPFN |
| 39204 | QDEMDGYDQ |
| 39205 | QDFLPVLKW |
| 39206 | QDHCEPFEP |
| 39207 | QDHKDECNE |
| 39208 | QDHLADWRN |
| 39209 | QDKHEPGKW |
| 39210 | QDKMPCWQF |
| 39211 | QDMGEKLTH |
| 39212 | QDMHDKEPA |
| 39213 | QDPCSWLKN |
| 39214 | QDPHHNHRH |
| 39215 | QDPQWWMRC |
| 39216 | QDRYGVPEN |
| 39217 | QDTDEGDQC |
| 39218 | QENTPGYEG |
| 39219 | QEPTEITPM |
| 39220 | QKPSDEKRC |
| 39221 | QKQCDRTAW |
| 39222 | QMFMPYCPF |
| 39223 | QQPWCWWEK |
| 39224 | QRCDDWIHS |
| 39225 | QRCKPRCPM |
| 39226 | QRDPEDFET |
| 39227 | QRMHDCCDL |
| 39228 | QVECSWDKY |
| 39229 | RDAKDGGRP |
| 39230 | RDAYWAEDN |
| 39231 | RDCFWCIGE |
| 39232 | RDCHKGIKN |
| 39233 | RDDCWPFNP |
| 39234 | RDDLRLWPF |
| 39235 | RDDLWLWPF |
| 39236 | RDHNEKINY |
| 39237 | RDHNEQINC |
| 39238 | RDIKPWFEM |
| 39239 | RDLEKGDEG |
| 39240 | RDPAWIWDY |
| 39241 | RDTMFEWKF |
| 39242 | REEFPLCNQ |
| 39243 | REEKDWFKH |
| 39244 | RELDCMWEN |
| 39245 | REPHDAKNN |
| 39246 | REPYWCLGY |
| 39247 | REQLWVHPG |
| 39248 | REYPKAHRE |
| 39249 | RHDTWHDEG |
| 39250 | RKDYWSDDG |
| 39251 | RLEWWIHPM |
| 39252 | RNDFWPAGN |
| 39253 | RNQDWGDRQ |
| 39254 | RPEMRCFRN |
| 39255 | RPKCNLIEM |
| 39256 | RPNCYKAEH |
| 39257 | RPWEWWIRK |
| 39258 | RRPEWLKNY |
| 39259 | SDCHENDPH |
| 39260 | SDDWRDFDN |
| 39261 | SDENDVDDC |
| 39262 | SDENEMEPH |
| 39263 | SDEWPYYRQ |
| 39264 | SDNREWMRY |
| 39265 | SDPCKVLRH |
| 39266 | SDSADDPKF |
| 39267 | SDSKGRERV |
| 39268 | SDWDPSWDT |
| 39269 | SDYSELPQH |
| 39270 | SEHEPDITN |
| 39271 | SEKFGIPRD |
| 39272 | SEPPDWNTK |
| 39273 | SGPRTWIKK |
| 39274 | SHDIPVPIM |
| 39275 | SHHNPGPRT |
| 39276 | SHYPNRPKG |
| 39277 | SKDWPPFKA |
| 39278 | SKQYDPPKP |
| 39279 | SMPLWQDSN |
| 39280 | SMPMWWPKH |
| 39281 | SNERACEEV |
| 39282 | SQPTGWEEN |
| 39283 | SRDGDEPNH |
| 39284 | SREFWWDGT |
| 39285 | SREWDCGSH |
| 39286 | SRPPDWPRT |
| 39287 | TDDCYGPKG |
| 39288 | TDDHERGDC |
| 39289 | TDDNGEIRP |
| 39290 | TDDWYGPKC |
| 39291 | TDEIPEMRY |
| 39292 | TDFSDRLKW |
| 39293 | TDHCNWSRT |
| 39294 | TDHKDGFRI |
| 39295 | TDHNGEIRT |
| 39296 | TDNRWMDKW |
| 39297 | TDPPDGFDM |
| 39298 | TDSCEDCKI |
| 39299 | TEFECFWNT |
| 39300 | TESLEKGPM |
| 39301 | TREGPQLKC |
| 39302 | TRLKELLIW |
| 39303 | TWDRECEYD |
| 39304 | VDEFNVWKH |
| 39305 | VDFFDWLDH |
| 39306 | VDHITKWKF |
| 39307 | VDPCDDGNA |
| 39308 | VDQHERARN |
| 39309 | VDQRDMATD |
| 39310 | VDWFARWDS |
| 39311 | VEFLDEIRH |
| 39312 | VEKMREWPF |
| 39313 | VEPYEGVRM |
| 39314 | VLDCDNPVD |
| 39315 | VNPMAYTRN |
| 39316 | VRDCRWCIV |
| 39317 | VREFEEDKN |
| 39318 | VRPYADSRN |
| 39319 | VRRDDPPRP |
| 39320 | VRSCGGHDI |
| 39321 | VRSWDTDFE |
| 39322 | VYWREEIRI |
| 39323 | WDCEDGWQQ |
| 39324 | WDDHERGQV |
| 39325 | WDDRPALKG |
| 39326 | WDDRQCEQH |
| 39327 | WDEKPAGQV |
| 39328 | WDFCKCADI |
| 39329 | WDFNDGITP |
| 39330 | WDNFNPPKQ |
| 39331 | WDNWDDREP |
| 39332 | WDPGEGNNF |
| 39333 | WDPLEHRLH |
| 39334 | WDPSKVNIM |
| 39335 | WDQCGWHRQ |
| 39336 | WDRASWLEQ |
| 39337 | WDRCDCEIP |
| 39338 | WDRCPMPRN |
| 39339 | WDSCEGREP |
| 39340 | WDSHDFEIH |
| 39341 | WDSKRLNPP |
| 39342 | WDWIEVYRC |
| 39343 | WDYRNLNRQ |
| 39344 | WECQSRDED |
| 39345 | WEDEPPWKI |
| 39346 | WEEQRIPSE |
| 39347 | WEEWDMMRC |
| 39348 | WELPCEWTY |
| 39349 | WEPHPYQTN |
| 39350 | WFDSRKEEW |
| 39351 | WFECWTYPM |
| 39352 | WGCKENWNM |
| 39353 | WGDCDPRVG |
| 39354 | WGDCDYCEK |
| 39355 | WGHKPCKGY |
| 39356 | WGPCAFGQE |
| 39357 | WGPKPCRGY |
| 39358 | WGSCECHKC |
| 39359 | WGTKPCKGY |
| 39360 | WHANEVLKK |
| 39361 | WHDTNLEPH |
| 39362 | WHSWWTNIM |
| 39363 | WKACPLRET |
| 39364 | WKAYPWQPT |
| 39365 | WKCAEFRTC |
| 39366 | WKCPDKLDN |
| 39367 | WKDAEKKDD |
| 39368 | WKDHTVPDH |
| 39369 | WKDNESSEF |
| 39370 | WKDRDQCLH |
| 39371 | WKLVDLGIG |
| 39372 | WKSCGMDSL |
| 39373 | WKTTEAPRD |
| 39374 | WLDDRLEFF |
| 39375 | WNDYDEGEC |
| 39376 | WNPADGKQN |
| 39377 | WNPWDQLEF |
| 39378 | WPCPWFDHM |
| 39379 | WPPEPWCCC |
| 39380 | WPPWEWYED |
| 39381 | WPSCGCTVE |
| 39382 | WPVDSWIEN |
| 39383 | WQANHWDRT |
| 39384 | WQEDQVGRQ |
| 39385 | WQPWRHERH |
| 39386 | WQQFWWNEC |
| 39387 | WQYYPCIDK |
| 39388 | WRAPGPLEN |
| 39389 | WRCATIPIA |
| 39390 | WRCCQFWII |
| 39391 | WRCDDRDNC |
| 39392 | WRCPRLIDW |
| 39393 | WRDHPEDIQ |
| 39394 | WRDMDFYIN |
| 39395 | WRDQGFSTN |
| 39396 | WREMPMSEL |
| 39397 | WRHWDGLNL |
| 39398 | WRNQGDLVP |
| 39399 | WRNRPCLNE |
| 39400 | WRQCEFGVE |
| 39401 | WRSDGPIER |
| 39402 | WRSEEVEHC |
| 39403 | WRSRDHDHP |
| 39404 | WRTCDMPYF |
| 39405 | WRYAEWWNQ |
| 39406 | WRYTERIVH |
| 39407 | WWDCEPDTT |
| 39408 | WWDTDGNFG |
| 39409 | WYDNAGNKN |
| 39410 | WYPDEADHA |
| 39411 | WYQDWHPRM |
| 39412 | YDCCCGWKD |
| 39413 | YDCIPRCKP |
| 39414 | YDDAEMRGH |
| 39415 | YDEMYYCVG |
| 39416 | YDHRCYVRG |
| 39417 | YDNKPLPKH |
| 39418 | YDSCDCSRF |
| 39419 | YDTRWQPRC |
| 39420 | YDWKAADGW |
| 39421 | YDYRSYPVA |
| 39422 | YEAKETERP |
| 39423 | YEVDEEIKI |
| 39424 | YEVKEEPVS |
| 39425 | YGCFDRCMR |
| 39426 | YGDFEWCKR |
| 39427 | YGPEDLHKW |
| 39428 | YHDCRLHRP |
| 39429 | YKDARWPLW |
| 39430 | YKECHWHKE |
| 39431 | YKTVDVGPH |
| 39432 | YPNKDGERN |
| 39433 | YPWFDGWQW |
| 39434 | YQYFELPKW |
| 39435 | YRHEEYWHN |
| 39436 | YRHEPEDEG |
| 39437 | YRPGPTIRT |

### Example 43

AAV5 Variants with Thyroid Gland Tissue Tropism as Identified by Machine Learning

This example describes preferred properties of engineered AAV5 variants that display thyroid gland tissue tropism as identified by machine learning methods. The same bioinformatics pre-process, machine learning methods, and analyses as described in EXAMPLE 19 and FIGS. 19-FIG. 22 were carried out to identify preferred properties of engineered AAV5 variants that display thyroid gland tissue tropism.

Favored biophysical properties and favored amino acid residues at each position in the 581 to 589 region of an AAV5 VP capsid polypeptide, which are associated with a higher probability of thyroid gland tissue tropism are described below. Any of the below machine learning-derived positional amino acid preferences described in TABLE 70 can be present alone or in combination with each other or in combination with any of the amino acid preferences observed and described in EXAMPLE 15, TABLE 29. Listed below in TABLE 70 are a summary of positional features shared between the top important features for thyroid gland tropism extracted from the ML models.

TABLE 70
Machine Learning-Derived Thyroid Gland Tissue Tropism Rules

High mutability at Xaa1
Xaa1 is selected from N
Low surface accessibility at Xaa2
Xaa2 is selected from F, G, or M
High solubility at Xaa3
Xaa3 is selected from F
Low mutability at Xaa3
Xaa3 is selected from Y, F, L, or C
Medium mol mass at Xaa3
Xaa3 is selected from D, E, R, K, V, P, M, I, L, N, Q, T, or C
Low surface accessibility at Xaa3
Xaa3 is selected from V, I, L, or C
High goldman engelman steitz at Xaa4
Xaa4 is selected from L or V
Low surface accessibility at Xaa4
Xaa4 is selected from V, M, A, G, F, I, or L
Low mol mass at Xaa4
Xaa4 is selected from D, A, G, I, L, or N
High solubility at Xaa5
Xaa5 is selected from C, L, F, M, V, or Y
Or
Low solubility at Xaa5
Xaa5 is selected from D
Low average flexibility at Xaa5
Xaa5 is selected from F, M, or W
Low average flexibility at Xaa6
Xaa6 is selected from F, M, or W
High mutability at Xaa7
Xaa7 is selected from N
Low volume at Xaa7
Xaa7 is selected from P, N, or T
Low average flexibility at Xaa8
Xaa8 is selected from F, M, or W
Low surface accessibility at Xaa8
Xaa8 is selected from M, G, or F
Low mutability at Xaa9
Xaa9 is selected from R, K, P, H, or C
Low hydropathy at Xaa9
Xaa9 is selected from R

TABLE 71 below provides sequences of the 581 to 589 region for variant AAV VP1 capsid polypeptides that exhibited thyroid gland tissue tropism and comport to one or more of the rules provided in TABLE 70. The present disclosure, thus, provides for rAAVs composed of engineered AAV5 VP1 capsid polypeptides having a SEQ ID NO: 2, wherein the Xaa1 to Xaa9 region is any one of SEQ ID NO: 41438-SEQ ID NO: 42437, as disclosed in TABLE 71. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 71
Sequences of the 581 to 589 Region in AAV5 VP1
Capsid Polypeptide that Drive Thyroid Gland Tissue
Tropism

| SEQ | |
|---|---|
| ID | 581-589 |
| NO | Sequence |
| 41438 | AACAVWKGE |
| 41439 | AANWFTHDK |
| 41440 | ACWLWFYFG |
| 41441 | ADSADRVYN |
| 41442 | AFHHMMNTP |
| 41443 | AFKKPSLFR |
| 41444 | AFYNYEIMA |
| 41445 | AHAIMMRDF |
| 41446 | AHHALSFWR |
| 41447 | AHHEVSFWR |
| 41448 | AHNIMMRDF |
| 41449 | AHTIMMRDV |
| 41450 | AHTIMMRHF |
| 41451 | AHTIMMRYF |
| 41452 | AIHEPAENR |
| 41453 | AIHEPAGDR |
| 41454 | AIHKPAEDR |
| 41455 | AIHVPAEDR |
| 41456 | AKIPIDHFA |
| 41457 | AKSSIEFSA |
| 41458 | AKSSIEHSA |
| 41459 | AKSSIEYSG |
| 41460 | AMHEPAEDR |
| 41461 | AMMKMMKAN |
| 41462 | AMMLMMKAN |
| 41463 | AMMQMMKTN |
| 41464 | AMMQMMRAN |
| 41465 | ANERHWTSF |
| 41466 | ANVRHWTYF |
| 41467 | ARAMYVESF |
| 41468 | ARSSIEYSA |
| 41469 | ARTMYGESF |
| 41470 | ASDTMNCGS |
| 41471 | ASYHWTHAS |
| 41472 | ASYHWTHVG |
| 41473 | ATERHWTYF |
| 41474 | ATRDWMSCM |
| 41475 | AVDEVGSSN |
| 41476 | AVHEPAEDR |
| 41477 | AVILSSMYS |
| 41478 | AVTVDGNNR |
| 41479 | AWMDCPIMC |
| 41480 | AYDEKKNQM |
| 41481 | AYDKKKNQM |
| 41482 | AYDQRKNQM |
| 41483 | AYLINMLWM |
| 41484 | AYNQKKNQM |
| 41485 | CFENLQIMA |
| 41486 | CGGQFWQGI |
| 41487 | CKDEFIVGN |
| 41488 | CKIPIDHFA |
| 41489 | CKQKFNGQN |
| 41490 | CKQQFIGQN |
| 41491 | CKQQFTGQN |
| 41492 | CKQRFNGQN |
| 41493 | CMESYDHGS |
| 41494 | CMFGMVNYP |
| 41495 | CMFGTVNYT |
| 41496 | CMKSFDHGS |
| 41497 | CMQKDGNDR |
| 41498 | CMRGDKRKR |
| 41499 | CMRGDQRNR |
| 41500 | CMRGDTRNR |
| 41501 | CMRKDGNDR |
| 41502 | CMRMDCNDR |
| 41503 | CMRMDGNNR |
| 41504 | CMRMVGNDR |
| 41505 | CMRSDKRNR |
| 41506 | CMRVDGNDR |
| 41507 | CMVDANHVW |
| 41508 | CNFNNNDHV |
| 41509 | CTLFTNFQY |
| 41510 | CTLISNFQY |
| 41511 | CVGQFWEGI |
| 41512 | CVGQFWKGI |
| 41513 | CVGQFWRGI |
| 41514 | CVVQFWQGI |
| 41515 | DANDMSSDR |
| 41516 | DARFGINFG |
| 41517 | DARFGLNFA |
| 41518 | DARFYIAEG |
| 41519 | DARFYISEG |
| 41520 | DCVIDWCQM |
| 41521 | DDSARYWQV |
| 41522 | DDSSWDNLF |
| 41523 | DELKMGHNS |
| 41524 | DFEYTCNQH |
| 41525 | DFEYTCNQS |
| 41526 | DFQKPSLFR |
| 41527 | DGAHFRSCF |
| 41528 | DGIIDWCQM |
| 41529 | DGSQAGPDK |
| 41530 | DGVIDWCKM |
| 41531 | DGVIDWRQM |
| 41532 | DGVVDWCQM |
| 41533 | DHIACKKLC |
| 41534 | DHTACKKVC |
| 41535 | DHTACKRLC |
| 41536 | DHTGCKKLC |
| 41537 | DIFFKCEWV |
| 41538 | DIFFKCVWA |
| 41539 | DIHHLCNIF |
| 41540 | DIHHWCNIC |
| 41541 | DIHPWCNIF |
| 41542 | DINAKKNIC |
| 41543 | DINGKKNIR |
| 41544 | DINGKKNIW |
| 41545 | DINGKKNVC |
| 41546 | DINGRKNIC |
| 41547 | DIQHWCNIF |
| 41548 | DIYHFNHKS |
| 41549 | DIYHWCNIF |
| 41550 | DKEDDHRRA |
| 41551 | DKIAIDHFA |
| 41552 | DKIPIAHFA |
| 41553 | DKIPIDHFT |
| 41554 | DKIPMDHFA |
| 41555 | DKIPVDHFA |
| 41556 | DKISIDHFA |
| 41557 | DKLPIDHFA |
| 41558 | DKMLMVFKR |
| 41559 | DKMPIDHFA |
| 41560 | DKVPIDHFA |
| 41561 | DLHHWCNIF |
| 41562 | DLNGKKNIC |
| 41563 | DQLKMGHNI |
| 41564 | DRCFRDDFE |
| 41565 | DRCFRDDMV |
| 41566 | DRWTMQHWE |
| 41567 | DSGKANTQS |
| 41568 | DSKADHFVN |
| 41569 | DSKADHGVN |
| 41570 | DSKADNCVN |
| 41571 | DSKADPCVN |
| 41572 | DSKADRCVN |
| 41573 | DSKADYCVN |
| 41574 | DSMADDPHL |
| 41575 | DSMGDDQHL |
| 41576 | DSRFNESLG |
| 41577 | DTVPSHLGC |
| 41578 | DVLKMGHNI |
| 41579 | DVVIDWCQM |
| 41580 | DWIVDPPKP |
| 41581 | DWIVDTAKP |
| 41582 | DWIVDTPKA |
| 41583 | DWIVDTPKL |
| 41584 | DWIVDTPQP |
| 41585 | DWMDCSIMC |
| 41586 | ECERAKGEW |
| 41587 | ECERPKGEW |
| 41588 | ECERTKGGW |
| 41589 | ECERTKGKW |
| 41590 | ECERTKGQW |
| 41591 | ECERTRGEW |
| 41592 | ECKRTKGEW |
| 41593 | EDDGDDNIP |
| 41594 | EEIAGHVGA |
| 41595 | EFHQDMFEC |
| 41596 | EFSGYTNQE |
| 41597 | EFYDYEIMA |
| 41598 | EFYNYEIMT |
| 41599 | EFYNYGIMA |
| 41600 | EFYNYQIMA |
| 41601 | EHGAVHNAS |
| 41602 | EHGAVLNAN |
| 41603 | EHHGFNHCN |
| 41604 | EHMTCMHCE |
| 41605 | EIHQDMFEF |
| 41606 | EKAMLLNGT |
| 41607 | EKAMLMNGA |
| 41608 | EKAMVLNGA |
| 41609 | EKEDDHRRP |
| 41610 | EKFMVMNSC |
| 41611 | ELMWWQWDR |
| 41612 | EMSSEMEKL |
| 41613 | ERFMVMNSF |
| 41614 | ERFMVMNSW |
| 41615 | ERFMVMSSC |
| 41616 | ERGAVHNAN |
| 41617 | ERHAFNHCN |
| 41618 | ERHGFNHCK |
| 41619 | ERHGFNHCS |
| 41620 | ERHGFNHWN |
| 41621 | ERHGINHCN |
| 41622 | ERHGVNHCN |
| 41623 | ERQTLAMDR |
| 41624 | ESMWYYVMA |
| 41625 | ESSWPLQWN |
| 41626 | ESTISPNHK |
| 41627 | ESVDHWGDW |
| 41628 | ETAMGTNLY |
| 41629 | ETAMLLNGA |
| 41630 | ETAYRENPC |
| 41631 | ETGPSMFSF |
| 41632 | ETGYGENPC |
| 41633 | ETGYQENPC |
| 41634 | ETGYRENSC |
| 41635 | EVSKFWRHT |
| 41636 | EVTVDGNNR |
| 41637 | EVVAGHVGA |
| 41638 | EYCHWWYKL |
| 41639 | EYERLGDSW |
| 41640 | EYMNCMHCE |
| 41641 | FAEPCMAIW |
| 41642 | FCERNMMAQ |
| 41643 | FCNRNVNRP |
| 41644 | FKFWTDNAD |
| 41645 | FKFWTDNTE |
| 41646 | FKFWTDNTN |
| 41647 | FKSIQCSWG |
| 41648 | FKSVQCSWA |
| 41649 | FMVEFAYMD |
| 41650 | FNQTMIFGA |
| 41651 | FNVPDLLGA |
| 41652 | FPANYYSNA |
| 41653 | FPGNYHSNA |
| 41654 | FPKFCKCIS |
| 41655 | FSDTMNCGS |
| 41656 | FSQTMIFCA |
| 41657 | FTRDWMSCM |
| 41658 | FVEPCMAIL |
| 41659 | FVEPCMAIS |
| 41660 | FVEPCMTIW |
| 41661 | FVERCMAIW |
| 41662 | FVQPCMAIW |
| 41663 | GAYFFDKYK |
| 41664 | GAYFFDKYS |
| 41665 | GAYFFHKYN |
| 41666 | GCQGFFNMK |
| 41667 | GCQGFFNMP |
| 41668 | GDAALYNWF |
| 41669 | GDGSWDNLF |
| 41670 | GDSSLDNLF |
| 41671 | GDSSWDNVF |
| 41672 | GDSVLYNWF |
| 41673 | GDTSWDNLF |
| 41674 | GELKMGHNI |
| 41675 | GFTVDGNNR |
| 41676 | GGPKAANDW |
| 41677 | GIHHWCNIF |
| 41678 | GKMVMVFKR |
| 41679 | GMRGDKRNR |
| 41680 | GRFMVMNSC |
| 41681 | GSSWPLLWN |
| 41682 | GSYHWTHAG |
| 41683 | GTADCRNSS |
| 41684 | GTGEWMHMA |
| 41685 | GVAKAANDW |
| 41686 | GVNVDGNNR |
| 41687 | GVPKAANDL |
| 41688 | GVPRAANDW |
| 41689 | GVPVDGNNR |
| 41690 | GVSVDGNNR |
| 41691 | GVTGDGNNR |
| 41692 | GVTIDGNNR |
| 41693 | GVTLDGNNR |
| 41694 | GVTVDANNR |
| 41695 | GVTVDGNDR |
| 41696 | GWMDCSIMC |
| 41697 | GYLINMLLM |
| 41698 | GYLISMLWM |
| 41699 | GYLIYMLWM |
| 41700 | GYLTNMLWM |
| 41701 | GYRINMLWM |
| 41702 | HAGSGSNVL |
| 41703 | HAGTGSNVL |
| 41704 | HHICFQHCK |
| 41705 | HIHHWCNIF |
| 41706 | HKISLFKVN |
| 41707 | HNLMGWYGP |
| 41708 | HQTLDMERF |
| 41709 | HQTLDMKKF |
| 41710 | HQTLDMKRV |
| 41711 | HRCFRDDME |
| 41712 | HSAYFRVGA |
| 41713 | HTGPGSNVL |
| 41714 | HVEYVIWPG |
| 41715 | HVEYVIWPV |
| 41716 | HVKYVIWPD |
| 41717 | HWISFERHP |
| 41718 | HYEICHISK |
| 41719 | IADIVWQGQ |
| 41720 | IAHPFKCGG |
| 41721 | ICAIANTCN |
| 41722 | IDDDNWEQP |
| 41723 | IDDGDDNIP |
| 41724 | IEIALHACW |
| 41725 | IFAAPMPGM |
| 41726 | IFHSNPVVR |
| 41727 | IFHSTPIVR |
| 41728 | IFVAPMPGR |
| 41729 | IFVASMPGM |
| 41730 | IFWMKCGWR |
| 41731 | IGNRNVNRP |
| 41732 | IINWLYNEP |
| 41733 | IISIRMYQA |
| 41734 | IKFWTDNTD |
| 41735 | IMIEFAYMD |
| 41736 | IMLEFAYMD |
| 41737 | IMVEFADMD |
| 41738 | IMVEFSYMD |
| 41739 | IMVEFTYMD |
| 41740 | INDCCTMPD |
| 41741 | INDCCTMSD |
| 41742 | INVPDILGA |
| 41743 | INVPDLLGG |
| 41744 | INVPDLLGS |
| 41745 | IPKFCKCSS |
| 41746 | IPKVCKCIS |
| 41747 | IPQFCKCIS |
| 41748 | IPWEYAMND |
| 41749 | IPWPYAMND |
| 41750 | ISDCFTCAD |
| 41751 | ISGMKSAMA |
| 41752 | ITETPGNTH |
| 41753 | ITGAKMYMG |
| 41754 | ITGAMMHMG |
| 41755 | ITGATMYMG |
| 41756 | ITGIRMYQA |
| 41757 | ITSIGMYQA |
| 41758 | ITSIRMDQA |
| 41759 | ITSIRMFQA |
| 41760 | ITSIRMYKA |
| 41761 | ITSIRMYRA |
| 41762 | ITSIWMYQA |
| 41763 | ITSLRMYQA |
| 41764 | ITSNRMYQA |
| 41765 | IVGADRCQA |
| 41766 | IVNACMAEK |
| 41767 | IVQSFWNIP |
| 41768 | IVVAVNQSL |
| 41769 | IWDFKCNQC |
| 41770 | IWEDYLHVG |
| 41771 | IWKDYLHMG |
| 41772 | IWKDYLHVA |
| 41773 | IWKDYLYVG |
| 41774 | IYEERNAQM |
| 41775 | IYICGPMIN |
| 41776 | IYIREPMIN |
| 41777 | IYIRGPMLN |
| 41778 | IYIRVPMIN |
| 41779 | IYLRGPMIN |
| 41780 | IYRLREKQE |
| 41781 | IYTRGPMIN |
| 41782 | IYVRGPMIN |
| 41783 | KAFGWFHFD |
| 41784 | KAIGWFHFH |
| 41785 | KAIGWFHFN |
| 41786 | KAIGWFYFD |
| 41787 | KALGWFHFD |
| 41788 | KAMGWFHFD |
| 41789 | KAVAVNQSL |
| 41790 | KDAGDDNIP |
| 41791 | KDDADDNIP |
| 41792 | KDDGDDNIL |
| 41793 | KDDGDDNIQ |
| 41794 | KDDGDNNIP |
| 41795 | KDDGGDNIP |
| 41796 | KDGGDDNIP |
| 41797 | KDNGDDNIP |
| 41798 | KDRMLAGQR |
| 41799 | KDSHVHSFF |
| 41800 | KDYGDDNIP |
| 41801 | KECDPQHWW |
| 41802 | KEMRKQGMW |
| 41803 | KEYAPQHWW |
| 41804 | KEYDLQHWW |
| 41805 | KEYDPEHWW |
| 41806 | KEYDPQNWW |
| 41807 | KEYDPQYWW |
| 41808 | KEYDPRHWW |
| 41809 | KFSGYTNQE |
| 41810 | KFYNYEIMA |
| 41811 | KGRWMYHWW |
| 41812 | KHCHWWYKL |
| 41813 | KIEMLCCPG |
| 41814 | KIEMLCCQR |
| 41815 | KIEMLCCQV |
| 41816 | KIHQDMFEC |
| 41817 | KIKMLCCQG |
| 41818 | KIMWWQWDR |
| 41819 | KKRRDTHIY |
| 41820 | KLEMLCCQG |
| 41821 | KLYGRRHWH |
| 41822 | KMQNENRQM |
| 41823 | KNDGDDNIP |
| 41824 | KPEWTMMYD |
| 41825 | KPQWAMMYD |
| 41826 | KPYEDIARM |
| 41827 | KQEMQGVCR |
| 41828 | KQYDPQHWW |
| 41829 | KRETLAMDR |
| 41830 | KRFMVMNSC |
| 41831 | KRHGFNHCN |
| 41832 | KSFYMCQCG |
| 41833 | KSFYMCRCA |
| 41834 | KSFYMFRCG |
| 41835 | KSSMLEHSW |
| 41836 | KSTMCDNDA |
| 41837 | KTEMSDMNW |
| 41838 | KTEMSDMYW |
| 41839 | KTYVWEHES |
| 41840 | KVLICQNGQ |
| 41841 | KVVAVDQSL |
| 41842 | KVVAVNRSL |
| 41843 | KWEQEPNRN |
| 41844 | KWRQAEYDE |
| 41845 | KWTRTGSSF |
| 41846 | KYCHWWYRL |
| 41847 | KYCLWWYKL |
| 41848 | KYCNWWYKL |
| 41849 | KYRHWWYKL |
| 41850 | KYWHWWYKL |
| 41851 | LAETPGNTH |
| 41852 | LAIALHACW |
| 41853 | LAIPQPVNG |
| 41854 | LATTMQGEN |
| 41855 | LATTMQPFN |
| 41856 | LATTMRAFN |
| 41857 | LCAEFPHCR |
| 41858 | LCAEYPHGR |
| 41859 | LCTEFPHGR |
| 41860 | LDALPMTQA |
| 41861 | LEIAMHACW |
| 41862 | LEINWWYPG |
| 41863 | LELALHACW |
| 41864 | LELNWWYPG |
| 41865 | LEVNWWFPG |
| 41866 | LEVNWWYAG |
| 41867 | LEVNWWYPA |
| 41868 | LEVNWWYPV |
| 41869 | LEVNWWYQG |
| 41870 | LFAEFPHGR |
| 41871 | LFTAFRKFT |
| 41872 | LFTAFRQFA |
| 41873 | LFTAFRQFN |
| 41874 | LFWMKCGWR |
| 41875 | LGDYVNDEG |
| 41876 | LGERTMDRI |
| 41877 | LHTIDANRR |
| 41878 | LIMRIPPED |
| 41879 | LIMRISSFD |
| 41880 | LIMRLSPFD |
| 41881 | LIVRISPFD |
| 41882 | LKNTMWKCA |
| 41883 | LKSCIQFSC |
| 41884 | LKVNWWYPG |
| 41885 | LLKFIHLNE |
| 41886 | LLKIYESNQ |
| 41887 | LMEFTEDCN |
| 41888 | LMEFTEDGD |
| 41889 | LMEFTEDGS |
| 41890 | LMEFTEEGN |
| 41891 | LMEITEDGN |
| 41892 | LMEPYGNCD |
| 41893 | LMRGTVKNG |
| 41894 | LMRSTVRNG |
| 41895 | LMSCIKFSC |
| 41896 | LMSCIPFSC |
| 41897 | LMSCIQISC |
| 41898 | LMSCLQFSC |
| 41899 | LNGMKSAMA |
| 41900 | LNVPDLLGA |
| 41901 | LPKFCKCIS |
| 41902 | LPRCFIAAC |
| 41903 | LPRCVIAAY |
| 41904 | LPRYVIAAC |
| 41905 | LSCCANVFC |
| 41906 | LSCCNNVFC |
| 41907 | LSCCTNGFC |
| 41908 | LSCCTNIFC |
| 41909 | LSDFVNDEG |
| 41910 | LSDHSQAAV |
| 41911 | LSDYVNEEG |
| 41912 | LSDYVNNEG |
| 41913 | LSDYVTDEG |
| 41914 | LSLSGITQL |
| 41915 | LSSCRGMIA |
| 41916 | LSYHTPVMA |
| 41917 | LTAREKKCG |
| 41918 | LTDYVNDEG |
| 41919 | LTEIPGNTH |
| 41920 | LTEKPGNTH |
| 41921 | LTEPPGNTH |
| 41922 | LTERPGNTH |
| 41923 | LTETPGNTQ |
| 41924 | LTQEDFTQM |
| 41925 | LTQTPGNTH |
| 41926 | LTRADFTQM |
| 41927 | LTREDFAQM |
| 41928 | LTREDFNQM |
| 41929 | LTREDFSQM |
| 41930 | LTREDITQM |
| 41931 | LTRQDFTQM |
| 41932 | LTRVDFTQM |
| 41933 | LTSIRMYQA |
| 41934 | LVGADRCQA |
| 41935 | LWCNENAMA |
| 41936 | LWCNENAME |
| 41937 | LWCNENAMG |
| 41938 | LWCNENAMN |
| 41939 | LWCNENPMD |
| 41940 | LWCTENAMD |
| 41941 | LWDFKCTQC |
| 41942 | LWGNENAMD |
| 41943 | LYAERNAQM |
| 41944 | LYEERHAQM |
| 41945 | LYEERNSQM |
| 41946 | LYEQRNAQM |
| 41947 | LYHHDILQT |
| 41948 | LYKERNAQM |
| 41949 | LYQERNAQM |
| 41950 | LYRWIVHNS |
| 41951 | LYTEYITKH |
| 41952 | LYYIAEHST |
| 41953 | LYYIPEHST |
| 41954 | MAGYLEKPF |
| 41955 | MAVTQSHQF |
| 41956 | MFVAPMPGM |
| 41957 | MGAYIMWDC |
| 41958 | MHVFSNVFG |
| 41959 | MIANYEGEM |
| 41960 | MIGNYEGEM |
| 41961 | MIHLPFGKR |
| 41962 | MIHLPFGQR |
| 41963 | MIHMPFGER |
| 41964 | MIVIYEGEM |
| 41965 | MIVNYEGEV |
| 41966 | MIVNYEGKM |
| 41967 | MIVRCSNTW |
| 41968 | MIVSYEGEM |
| 41969 | MLEQEPNRN |
| 41970 | MLFAENNGL |
| 41971 | MSSMLEHAW |
| 41972 | MSSMLERSW |
| 41973 | MTARRESAY |
| 41974 | MTARRESSY |
| 41975 | MTARRESTS |
| 41976 | MTARRGSTY |
| 41977 | MWEKEPNRN |
| 41978 | MWEQEANRN |
| 41979 | MWEQEPNQN |
| 41980 | MWFAANNGL |
| 41981 | MWGWPGDSF |
| 41982 | NAALYGGWI |
| 41983 | NACTGTWWR |
| 41984 | NAELFGGWI |
| 41985 | NAELSGGWI |
| 41986 | NAELYGGWV |
| 41987 | NAEQYGGWI |
| 41988 | NAEVYGGWI |
| 41989 | NAGLYGGWI |
| 41990 | NALVLQNVR |
| 41991 | NANDMGSDR |
| 41992 | NANDMSGDR |
| 41993 | NANDMSSDH |
| 41994 | NANDMSSNR |
| 41995 | NANDMSSYR |
| 41996 | NANVLSSDR |
| 41997 | NANVMSSDR |
| 41998 | NAQHAWENG |
| 41999 | NAVLYGGWI |
| 42000 | NAYENSHYP |
| 42001 | NAYLVHIPR |
| 42002 | NAYQCSHYP |
| 42003 | NAYQNSNYP |
| 42004 | NAYQTSHYP |
| 42005 | NCALERMPS |
| 42006 | NCAPARMPS |
| 42007 | NCAQERMPS |
| 42008 | NCLVNLCTY |
| 42009 | NCPPERMPS |
| 42010 | NCVVHLCTY |
| 42011 | NCVVNLCKY |
| 42012 | NCVVNPCTY |
| 42013 | NEAPMGLKY |
| 42014 | NEHPMGLKY |
| 42015 | NERPMGLKY |
| 42016 | NESPMGLKY |
| 42017 | NFWDQVHCV |
| 42018 | NFWNQVHCG |
| 42019 | NFWNQVHFV |
| 42020 | NFWSQVHCV |
| 42021 | NGAPERMPS |
| 42022 | NGCFVYWWL |
| 42023 | NGFMPAVMA |
| 42024 | NGRLLWQRY |
| 42025 | NGRLWWERY |
| 42026 | NGRLWWKRY |
| 42027 | NGRLWWQRC |
| 42028 | NGRLWWQRF |
| 42029 | NGVIINQCF |
| 42030 | NGVINDCEF |
| 42031 | NGVNMNQCF |
| 42032 | NGVSINQCF |
| 42033 | NGWLWWQRY |
| 42034 | NGYLVDIPR |
| 42035 | NGYLVNIPR |
| 42036 | NGYLVYIPR |
| 42037 | NHADYMRDA |
| 42038 | NHADYMRHA |
| 42039 | NHADYMRKA |
| 42040 | NHADYMRNV |
| 42041 | NHTACKKLC |
| 42042 | NHVDANLRA |
| 42043 | NHVNANRRA |
| 42044 | NHVNDNLRA |
| 42045 | NHVNTNLRA |
| 42046 | NIMSYSSWN |
| 42047 | NKMVMVFKR |
| 42048 | NLKWDWQNH |
| 42049 | NLKWREHHP |
| 42050 | NLKWRETHP |
| 42051 | NMCEYANVQ |
| 42052 | NMCKCANVQ |
| 42053 | NMCKFANVQ |
| 42054 | NMCKYANVP |
| 42055 | NMCKYANVR |
| 42056 | NMCKYPNVQ |
| 42057 | NMCKYSNVQ |
| 42058 | NMCRYANVQ |
| 42059 | NMHWQYQFE |
| 42060 | NMHWRYKFE |
| 42061 | NMIPMDSMP |
| 42062 | NMLPFNCNF |
| 42063 | NMPWRYQFE |
| 42064 | NMYWRYQFE |
| 42065 | NNCNMNVMR |
| 42066 | NNMHSDWCV |
| 42067 | NNMWCQKCP |
| 42068 | NNVNANLRA |
| 42069 | NPSQMDMDT |
| 42070 | NQAHCILNC |
| 42071 | NQLQMWYRV |
| 42072 | NQTHCIINC |
| 42073 | NRFASWEGM |
| 42074 | NSDEESGME |
| 42075 | NSLHNNEDA |
| 42076 | NSLHNNEHP |
| 42077 | NSLWCQKCP |
| 42078 | NSMWCEKCP |
| 42079 | NSMWCQECP |
| 42080 | NSMWCQKCL |
| 42081 | NSMWCQKCR |
| 42082 | NSMWCQKCT |
| 42083 | NSMWCQQCP |
| 42084 | NSMWCRKCP |
| 42085 | NSRHNNEDP |
| 42086 | NSSCIQECW |
| 42087 | NSSLIQECW |
| 42088 | NSSWIEECW |
| 42089 | NSSWIQECL |
| 42090 | NSSWIQQCW |
| 42091 | NSSWLQECW |
| 42092 | NSVHNNEDP |
| 42093 | NTADCRNSS |
| 42094 | NTDEESGMP |
| 42095 | NTKYAERSG |
| 42096 | NTQFAERSG |
| 42097 | NTQYADRSG |
| 42098 | NTQYAERCG |
| 42099 | NTQYAERPG |
| 42100 | NTQYAERSA |
| 42101 | NTQYSERSG |
| 42102 | NTRFADQTE |
| 42103 | NTRFPDQAE |
| 42104 | NTRFPDQTA |
| 42105 | NTRFSDQTE |
| 42106 | NTSWIQECW |
| 42107 | NTYQNSHYP |
| 42108 | NVFVAYCKY |
| 42109 | NVIVAYCKS |
| 42110 | NVLAFNCNF |
| 42111 | NVLPFNCHF |
| 42112 | NVLPFNGNF |
| 42113 | NVLPVNCNF |
| 42114 | NVLSHTMME |
| 42115 | NVLVHTMME |
| 42116 | NVMGHTMME |
| 42117 | NVNDMSSDR |
| 42118 | NVQCNRANG |
| 42119 | NVQCTRADG |
| 42120 | NVQCTRSNG |
| 42121 | NVYVAYCKS |
| 42122 | NWADSPWCY |
| 42123 | NWEHCWWAH |
| 42124 | NWENSPWCY |
| 42125 | NWEPCWWAY |
| 42126 | NWEPCWWDH |
| 42127 | NWEPCWWSH |
| 42128 | NWEPCWWTH |
| 42129 | NWERCWWAH |
| 42130 | NWVDSPWCY |
| 42131 | PDRMLAGQR |
| 42132 | PHICFQHCK |
| 42133 | PHTIMMRDF |
| 42134 | PIAKFANMS |
| 42135 | PLDWKLNNR |
| 42136 | PLDWPLNNR |
| 42137 | PLYGRRHWH |
| 42138 | PNILMMFEA |
| 42139 | PNIVMMFEP |
| 42140 | PNMLMMFEP |
| 42141 | PSPHEWMAF |
| 42142 | PSRPCTYHT |
| 42143 | PWAQTEKPN |
| 42144 | PWGETEKPN |
| 42145 | PWIPNQIKR |
| 42146 | PWTETEKPN |
| 42147 | QAWWKIFWH |
| 42148 | QEMRKKGMW |
| 42149 | QEMRKRGMW |
| 42150 | QEMRQQGMW |
| 42151 | QFSGYTNQV |
| 42152 | QFYNYEIMA |
| 42153 | QGQWMYHWW |
| 42154 | QGRWMYNWW |
| 42155 | QGRWMYYWW |
| 42156 | QGRWVYHWW |
| 42157 | QIHQDMFEC |
| 42158 | QIMRISPFD |
| 42159 | QIYGRRHWH |
| 42160 | QKAMLLNGA |
| 42161 | QLYGRRHLH |
| 42162 | QMSSEMEQL |
| 42163 | QNSNVMGCM |
| 42164 | QRHGFNHCN |
| 42165 | QSKWYYVMA |
| 42166 | QSRAGYMHY |
| 42167 | QSVDHWGDL |
| 42168 | QVHEWVFPR |
| 42169 | QVLICKNGQ |
| 42170 | QVLICQNAQ |
| 42171 | QVLICQNGL |
| 42172 | QVLICRNGQ |
| 42173 | QVLVCQNGQ |
| 42174 | QVVICQNGQ |
| 42175 | QVVIDHPKY |
| 42176 | QWIRTGSSF |
| 42177 | QWTRAGSSF |
| 42178 | QYCHWWYKL |
| 42179 | RAIGWFHFD |
| 42180 | REISVKAVA |
| 42181 | REISVQAVA |
| 42182 | REMRKQGMW |
| 42183 | RFFAAELQL |
| 42184 | RFFAAEPQW |
| 42185 | RFFAAQLQW |
| 42186 | RFFADELQW |
| 42187 | RFFAVELQW |
| 42188 | RHICFKHCK |
| 42189 | RHMCFQHCK |
| 42190 | RMATMFTCR |
| 42191 | RMHDACFDN |
| 42192 | RMHDACYDT |
| 42193 | RMHDGCYDN |
| 42194 | RMHDTCYDN |
| 42195 | RMNDACYDN |
| 42196 | RMRDACYDN |
| 42197 | RMYDACYDN |
| 42198 | RPEYANHMH |
| 42199 | RQTLDMKRF |
| 42200 | RSFYMCRCG |
| 42201 | RTEYADHMH |
| 42202 | RTKYANHMH |
| 42203 | RTYLWEHES |
| 42204 | RWFAENNGL |
| 42205 | RWRCRMRGN |
| 42206 | RYFAAELQW |
| 42207 | SANDMSSDR |
| 42208 | SANWCTHDK |
| 42209 | SANWFAHDK |
| 42210 | SASWFTHDK |
| 42211 | SATWFTHDK |
| 42212 | SCNIVEQVH |
| 42213 | SDAVLYNWF |
| 42214 | SEDIDYVHL |
| 42215 | SEYAAMRDP |
| 42216 | SEYAAMRDT |
| 42217 | SEYAAMRDY |
| 42218 | SEYAGMRDS |
| 42219 | SFTLLVGDY |
| 42220 | SGCAMHNMG |
| 42221 | SGCAMLDMG |
| 42222 | SGCAMLHMG |
| 42223 | SGCAMLNFG |
| 42224 | SGCAMLNMA |
| 42225 | SGCAMLNME |
| 42226 | SGCAMLNMV |
| 42227 | SGCAMLNVG |
| 42228 | SGCAMLYMG |
| 42229 | SGCAMPNMG |
| 42230 | SGCAMVNMG |
| 42231 | SGCGMLNMG |
| 42232 | SGCVMLNMG |
| 42233 | SGDYYDGMP |
| 42234 | SGFASWEGM |
| 42235 | SGLGMPNWP |
| 42236 | SGWAMLNMG |
| 42237 | SHMMNHAFW |
| 42238 | SHMMSNAFW |
| 42239 | SIKMCQSTV |
| 42240 | SIKVCESTV |
| 42241 | SIKVCQSNV |
| 42242 | SIKVCRSTV |
| 42243 | SIPSIGSEE |
| 42244 | SIVDFESAA |
| 42245 | SKSSIEYSA |
| 42246 | SLHSIGSEE |
| 42247 | SLPSIGNEE |
| 42248 | SLPSLGSEE |
| 42249 | SLPSVGSEE |
| 42250 | SMCKYANVQ |
| 42251 | SMIPMDSMT |
| 42252 | SMIPMDSVP |
| 42253 | SMSPMDSMP |
| 42254 | SMVPMDSMP |
| 42255 | SNERHWTYF |
| 42256 | SNMMSHAFW |
| 42257 | SNWWFMFEA |
| 42258 | SPQHCIGMP |
| 42259 | SRFASWDGM |
| 42260 | SRFASWEGV |
| 42261 | SSAYYDGMP |
| 42262 | SSDFYDGMP |
| 42263 | SSDYYAGMP |
| 42264 | SSDYYDGMT |
| 42265 | STAACRNSS |
| 42266 | STADCRNNS |
| 42267 | STADCRSSS |
| 42268 | STADGRNSS |
| 42269 | STADYRNSS |
| 42270 | STAGCRNSS |
| 42271 | STGDCRNSS |
| 42272 | STPDCRNSS |
| 42273 | STSADPNCG |
| 42274 | STSADPYCG |
| 42275 | STSGDPHCG |
| 42276 | STTDCRNSS |
| 42277 | STTFASSWE |
| 42278 | STWDWMSCM |
| 42279 | SVIQNEMRI |
| 42280 | SVLGHTMME |
| 42281 | SVLQNEMRI |
| 42282 | SVNMRFAKH |
| 42283 | SVNMRFTEH |
| 42284 | SVNWFTHDK |
| 42285 | SVVQNEMPI |
| 42286 | SVVQTEMRI |
| 42287 | SWIDMPMMQ |
| 42288 | SWTALGSVT |
| 42289 | SWTALSSVP |
| 42290 | SWTAVGSVP |
| 42291 | SWTIVEQVH |
| 42292 | SYLINMLWM |
| 42293 | SYMMSHAFW |
| 42294 | TACAGTWWR |
| 42295 | TASAIDHPK |
| 42296 | TASGIDHPQ |
| 42297 | TASGIDHPT |
| 42298 | TASGMDHPK |
| 42299 | TAVINDCEF |
| 42300 | TAWIYWNEF |
| 42301 | TDDGDDNIP |
| 42302 | TDRMMAGQR |
| 42303 | TFVAPMPGM |
| 42304 | TGVINNCEF |
| 42305 | TGVLNDCEF |
| 42306 | TGVMNDCEF |
| 42307 | TGVTNDCEF |
| 42308 | TGWVYWNEF |
| 42309 | THHELSFWR |
| 42310 | TIHEPAEDR |
| 42311 | TKVNHIMRP |
| 42312 | TNDIWLQFS |
| 42313 | TNDIWWQFA |
| 42314 | TNDIWWQFC |
| 42315 | TNDIWWQFF |
| 42316 | TNDIWWQVS |
| 42317 | TNDIWWQYS |
| 42318 | TNKMLACSS |
| 42319 | TQDIYEHMQ |
| 42320 | TRFASWEGM |
| 42321 | TSEFVAISM |
| 42322 | TTEMSDMHW |
| 42323 | TTSADPHCG |
| 42324 | TTWVYWNEF |
| 42325 | TVSGIDHPK |
| 42326 | TVVINDCEF |
| 42327 | TVWVYWNEF |
| 42328 | TYDQKKNQM |
| 42329 | VCAEFPHGR |
| 42330 | VCAIVNTCN |
| 42331 | VCNRNVNRP |
| 42332 | VCWLWFYCG |
| 42333 | VCWLWFYFA |
| 42334 | VCWLWFYFV |
| 42335 | VCWVWFYFG |
| 42336 | VCWWWFYFG |
| 42337 | VFHSNPIVR |
| 42338 | VFVAPMPGM |
| 42339 | VFWMKCGWK |
| 42340 | VFWMQCGWR |
| 42341 | VGIPERAMP |
| 42342 | VGWLWFYFG |
| 42343 | VHKWDTLRD |
| 42344 | VHMGEKMWS |
| 42345 | VHMGQKMWN |
| 42346 | VIARCSNTW |
| 42347 | VIIRCSNTW |
| 42348 | VILRCSNTW |
| 42349 | VIVNYEGEM |
| 42350 | VIVRCANTW |
| 42351 | VIVRCPNTW |
| 42352 | VIVRCSNTG |
| 42353 | VIVRGSNTW |
| 42354 | VKFWTDNTD |
| 42355 | VLVRCSNTW |
| 42356 | VMEFTEDGN |
| 42357 | VNDCCTMRD |
| 42358 | VPDLFNQFC |
| 42359 | VPHPFKCGG |
| 42360 | VSDFKIYMG |
| 42361 | VSDTLNCGS |
| 42362 | VSFLPDEYC |
| 42363 | VSFLPNQYC |
| 42364 | VSYHWTHAG |
| 42365 | VTAFKIYMG |
| 42366 | VTAREEKCG |
| 42367 | VTDFEIYMG |
| 42368 | VTGAMMYMG |
| 42369 | VTIHERRCN |
| 42370 | VTSEWMHMA |
| 42371 | VTVHEHRCN |
| 42372 | VVICKRFVG |
| 42373 | VVTVDGNNR |
| 42374 | VWFAENNGL |
| 42375 | VWMDCSIMC |
| 42376 | VYEERNAQM |
| 42377 | VYRLRERQE |
| 42378 | VYRVREKQE |
| 42379 | WDCLLHKWR |
| 42380 | WDCLLNRWR |
| 42381 | WDCLVNKWR |
| 42382 | WECLLNKWR |
| 42383 | WGEACKLDT |
| 42384 | WHCLLNKWR |
| 42385 | WHGMRMIRA |
| 42386 | WHGMRMIRT |
| 42387 | WHGMRMLRP |
| 42388 | WHGMRMVRP |
| 42389 | WKVIYHQPG |
| 42390 | WKVIYHQRG |
| 42391 | WMEHYGNCD |
| 42392 | WMEPYDNCD |
| 42393 | WMEPYGNCE |
| 42394 | WMEPYGNCN |
| 42395 | WMEPYGNYD |
| 42396 | WMESYGNCD |
| 42397 | WMGPYGNCD |
| 42398 | WMKSYDHGS |
| 42399 | WMSKQMFKG |
| 42400 | WMSQQMFKG |
| 42401 | WNCLLNKWR |
| 42402 | WSSVAHPGN |
| 42403 | WVGQFWQGI |
| 42404 | WWAPPWHAL |
| 42405 | WWTDMSSWK |
| 42406 | WWTDTSSWT |
| 42407 | WWTNMSSWT |
| 42408 | WYGMRMIRP |
| 42409 | YGYLVHIPR |
| 42410 | YHEICHISK |
| 42411 | YHTACKKLC |
| 42412 | YKFWTDNTD |
| 42413 | YLKDVDFDS |
| 42414 | YPQCVQWFW |
| 42415 | YPRCAQWFW |
| 42416 | YQHWAMVQR |
| 42417 | YQHWTMDQR |
| 42418 | YVEYVIWPD |
| 42419 | YVSQMWMRC |
| 42420 | YVSQVWMQC |
| 42421 | YWLLDHANF |
| 42422 | YYAICHISK |
| 42423 | YYEFCHISK |
| 42424 | YYEICDISK |
| 42425 | YYEICHICK |
| 42426 | YYEICHIST |
| 42427 | YYEICHLSK |
| 42428 | YYEICNISK |
| 42429 | YYEICPISK |
| 42430 | YYEICQISK |
| 42431 | YYEICRISK |
| 42432 | YYEICYISK |
| 42433 | YYELCHISK |
| 42434 | YYGICHISK |
| 42435 | YYKICHISK |
| 42436 | YYQICHISK |
| 42437 | YYVICHISK |

### Example 44

Treatment of Rett Syndrome with an AAV5-Derived Virion Encapsidating a Therapeutic Payload

This example describes treatment of Rett syndrome with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a region from a position corresponding to 581 to a position corresponding to 589 in the VP1 capsid polypeptide and where the polynucleotide sequence encoding for the variant AAV5 Cap encodes for an amino acid sequence of any one of SEQ ID NO: 7118-SEQ ID NO: 10117. The therapeutic payload is a guide RNA targeting an mRNA encoded for by a gene implicated in Rett syndrome, a suppressor tRNA targeting a premature termination codon (PTC) in a gene implicated in Rett syndrome, or a transgene. The mRNA targeted by the guide RNA is an mRNA encoded for by a gene encoding for MECP2. The suppressor tRNA targets a PTC in MECP2. The transgene is MECP2. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared wildtype AAV5 virions encapsidating the therapeutic payload. Upon administration to the subject, a lower dose of the variant AAV5 virions encapsidating the therapeutic payload is administered as compared to wildtype AAV5 virions encapsidating the therapeutic payload. Upon administration to the subject, at least one symptom of Rett syndrome is alleviated or the subject is cured.

### Example 45

### Determination of Enriched Variants

This example illustrates determination of enriched variants in each of the following tissues: adrenal gland, bone marrow, CNS tissues, colon, heart, lung, lymph node, mammary gland, skeletal muscle or cardiac muscle, sciatic nerve, skeletal muscle, skin, spinal cord, spleen, and thyroid gland.

Capsid variants may be analyzed with respect to their observed abundance. Following filtering as described in EXAMPLE 19, variants are scored based on the number of repeated observations between animals, or tissue sections, or CNS regions, or sequencing samples. This results in a distribution of variant frequency and a variant may be ranked by its observation relative to the distribution of observational frequencies of all other variants through statistical methods.

Additionally, variant “enrichment” within a tissue may be calculated using the following equation, or variations of this equation:

$$Enrichment score = \frac{\begin{matrix}variant 1 UMIcount in tissue 1 / \\ sum of variants UMIcount in tissue 1\end{matrix}}{\begin{matrix}variant 1 UMIcount in all tissues / \\ sum of variants UMIcount in all tissues\end{matrix}}$$

$$\frac{VAR jk / {\sum }_{j = 1}^{n} VAR jk}{{\sum }_{k = 1}^{m} VAR jk / {\sum }_{k = 1}^{m} {\sum }_{j = 1}^{n} VAR jk}$$

where VAR is the UMI count for variant j in tissue k for all n variants in all m tissues.

As with observations, this results in a distribution of variant enrichment frequency within each tissue and a variant may be ranked by its enrichment relative to the distribution of enrichment frequencies of all other variants through statistical methods.

The resulting observational and enrichment relative scores may then be summed, or averaged, or otherwise integrated, and variants are ranked by this integrated observation/enrichment score to identify candidates likely to infect a given tissue.

To generate the list of capsid variants present in each tissue based on the integrated observational and enrichment scores, preference was given to variants observed. Variants were ranked by the integrated score and up to 1000 of the top variants were included.

Enriched sequences for each tissue are described in the tables below. Also encompassed herein are rAAVs composed of engineered AAV5 VP2 capsid polypeptides and engineered AAV5 VP3 capsid polypeptides having the sequences disclosed in the below table at the regions in AAV5 VP2 (amin acid residues 445 to 453) and AAV5 VP3 (amino acid residues 389-397) corresponding to the amino acids in the AAV5 VP1 581 to 589 region.

TABLE 72
Sequences of the 581 to 589 Region in
AAV5 VP1 Capsid Polypeptide that Drive Adrenal
Gland Tissue Tropism

| | 581-589 |
|---|---|
| SEQ ID NO | Sequence |
| 3118 | KIEYGHWIH |
| 3119 | TIDVKRWGQ |
| 3120 | CAIPRHVFP |
| 3121 | CSYKNQQHQ |
| 3122 | AAQKGQHYS |
| 3123 | AEMPTVNNI |
| 3124 | AERTKELHS |
| 3125 | AGWHNTFWG |
| 3126 | AHDAYTKNS |
| 3127 | ANVHSGQCQ |
| 3128 | AQVFSVGWT |
| 3129 | ASHRYILAE |
| 3130 | ATMTFHFSP |
| 3131 | ATVLEASPD |
| 3132 | AVHLKAACP |
| 3133 | AVMHKNADY |
| 3134 | AVMSIPFQS |
| 3135 | AVQSCWTGT |
| 3136 | CFHRCLFIL |
| 3137 | CHLSTCRWP |
| 3138 | CIHMLKLAN |
| 3139 | CIHQYGRVH |
| 3140 | CILRHIKEN |
| 3141 | CKMRQRPFR |
| 3142 | CLIRNVAGG |
| 3143 | CPHKFLMER |
| 3144 | CPRCYHRAQ |
| 3145 | CSGSKMRMG |
| 3146 | CTANRAMML |
| 3147 | CTQRRMTFP |
| 3148 | CTSYRLSTP |
| 3149 | CVMSYINWH |
| 3150 | CVNYNLGND |
| 3151 | CYKWIDMCN |
| 3152 | DHEWYCSLA |
| 3153 | DILRYHTQL |
| 3154 | DSFQTENSM |
| 3155 | DTWIMSQVH |
| 3156 | DVKAPHGCV |
| 3157 | EDGRADWHV |
| 3158 | EHDCYMAVV |
| 3159 | ETALLEFRR |
| 3160 | EVCQDYQQL |
| 3161 | EWKRRLKRR |
| 3162 | FCHLIMRYY |
| 3163 | FCNYVATDD |
| 3164 | FHLRSHWMP |
| 3165 | GECRESCVT |
| 3166 | GKSDCEHMY |
| 3167 | GMHETSVND |
| 3168 | GPPLFTMIG |
| 3169 | GTTQARIGS |
| 3170 | GTVCNNECR |
| 3171 | GYIQVHVFN |
| 3172 | GYQRAYVPF |
| 3173 | HALTWMRAK |
| 3174 | HFPYNFWYY |
| 3175 | HKIYGSTPY |
| 3176 | HSQPDKTGA |
| 3177 | HSYNWDEGG |
| 3178 | HVMTRNNVY |
| 3179 | IIQKTQHNT |
| 3180 | IKVQMECHM |
| 3181 | ILYNHWVIG |
| 3182 | IMEEARTYC |
| 3183 | IMSNYCLTA |
| 3184 | INKVQTFGL |
| 3185 | IRCCTKPLT |
| 3186 | KCATYPSHD |
| 3187 | KIGMEEVMS |
| 3188 | KILIRDCGV |
| 3189 | KKKYWKMED |
| 3190 | KMLYSSYTQ |
| 3191 | KNEWPCIGF |
| 3192 | KNKTSEYAC |
| 3193 | KTFFHLSFA |
| 3194 | KWADQRNSF |
| 3195 | LCMLAQIME |
| 3196 | LLQKMWAPT |
| 3197 | LMNRRADER |
| 3198 | LVHCFLKSA |
| 3199 | LYCLCKNWC |
| 3200 | LYGLKMMYN |
| 3201 | MANMVWQNW |
| 3202 | MGAMALELA |
| 3203 | MSKIKQRLN |
| 3204 | MVNRPATCV |
| 3205 | MVTPHDHWH |
| 3206 | NCDSKVVGH |
| 3207 | NCLKEIVVD |
| 3208 | NHHCCTPSP |
| 3209 | NHRWNFGKS |
| 3210 | NLVDCECRV |
| 3211 | NTCEGWSHF |
| 3212 | PHMPYSDQQ |
| 3213 | PQYSVCGGP |
| 3214 | PSARSEQQD |
| 3215 | QAGGSGFNE |
| 3216 | QISAYGRPL |
| 3217 | QQNGSNNFM |
| 3218 | QSTPTLGPK |
| 3219 | QVGTHNHMI |
| 3220 | QWVHKQNLD |
| 3221 | QYADFKAQQ |
| 3222 | QYYMTIHHF |
| 3223 | RAETTKQLM |
| 3224 | RCWITEVSY |
| 3225 | RELSVQQTI |
| 3226 | RGKGHGTDL |
| 3227 | RGLKCYMRN |
| 3228 | RVHTIDLTH |
| 3229 | RYQPVENIE |
| 3230 | SCCWRGSME |
| 3231 | SHVCQEYMN |
| 3232 | SLPQTDQHV |
| 3233 | SNAEMSQLF |
| 3234 | SNKLMMDVM |
| 3235 | SPRCGGPFP |
| 3236 | SVCMHIGSQ |
| 3237 | SVGWSFEPK |
| 3238 | TDLEQGMDQ |
| 3239 | TFDLWQWGH |
| 3240 | TTEWKPSIL |
| 3241 | TYLHQPEDS |
| 3242 | TYSNDGLFN |
| 3243 | VAKITMNTW |
| 3244 | VCQVGFLND |
| 3245 | VGQPTEKSF |
| 3246 | VHCKVWGQC |
| 3247 | VMEGQRGWT |
| 3248 | VSAYRCSFE |
| 3249 | VVMHCAKEF |
| 3250 | VYHRLCRDT |
| 3251 | VYHRNATWV |
| 3252 | WPEQCNVNL |
| 3253 | YAQSVSESN |
| 3254 | YCERCQDFH |
| 3255 | YDEYPRNAT |
| 3256 | YDNYWAYNP |
| 3257 | YGVCLWNNG |
| 3258 | YITQQYASH |
| 3259 | YNPQQGKSK |
| 3260 | YPYAILVDI |
| 3261 | YWQWNTSGN |
| 3262 | YWVPDCYNF |
| 3263 | GVHATCMHA |
| 3264 | ADRQCLQGP |
| 3265 | ASDPNRWAG |
| 3266 | ATGTYNLQK |
| 3267 | CIHRYSKSS |
| 3268 | CIHWYGKSS |
| 3269 | CIHWYSKSA |
| 3270 | CIHWYSKSS |
| 3271 | CIHWYSKSY |
| 3272 | CLVSYSQGQ |
| 3273 | CLVSYSRGQ |
| 3274 | CMHWYSKSS |
| 3275 | CMQTDKLMN |
| 3276 | CMQTGKLMN |
| 3277 | CMQTVELMN |
| 3278 | CMQTVKLMK |
| 3279 | CMQTVKLMN |
| 3280 | CMQTVKLMS |
| 3281 | CMQTVKLMT |
| 3282 | CMQTVRLMN |
| 3283 | CSMARRYWG |
| 3284 | CSMTRGYWG |
| 3285 | CSMTRRYWG |
| 3286 | CSMTRRYWS |
| 3287 | CSPGMHLSP |
| 3288 | CSPSMHLSP |
| 3289 | CSPSMHPSP |
| 3290 | CSPSVHLSP |
| 3291 | CSVTRRYWG |
| 3292 | CTMYRNVSW |
| 3293 | CVGWQSTAP |
| 3294 | CVGWRSAAP |
| 3295 | CVGWRSTAP |
| 3296 | CVGWWSTAP |
| 3297 | CVHWYSKSS |
| 3298 | CVIPPRGCV |
| 3299 | CVIPPRGWV |
| 3300 | CVQWILNSA |
| 3301 | CVQWIRNSA |
| 3302 | CVQWIRNSV |
| 3303 | CYPSMHLSP |
| 3304 | DHDEIFVAR |
| 3305 | DHDEMFVAR |
| 3306 | DHNEIFVAR |
| 3307 | DIQCANEGY |
| 3308 | EGIHCWKEM |
| 3309 | EGIHCWKGM |
| 3310 | EIDPNRWAG |
| 3311 | ESAPNRWAG |
| 3312 | ESDPNRGAG |
| 3313 | ESDPNRLAG |
| 3314 | ESDPNRWAC |
| 3315 | ESDPNRWAD |
| 3316 | ESDPNRWAG |
| 3317 | ESDPNRWSG |
| 3318 | FSLFCGQYS |
| 3319 | FSLFCRQYS |
| 3320 | GICFALNAP |
| 3321 | GIQCANEGY |
| 3322 | GLTIDHQLV |
| 3323 | GLTTDHQLV |
| 3324 | GLTTDPQLV |
| 3325 | GLTTNHQLV |
| 3326 | GVQCANEGY |
| 3327 | IICFALNAP |
| 3328 | IMLSCHNMK |
| 3329 | IMLSCHNMR |
| 3330 | IMLSCRNMK |
| 3331 | IMLSYHNMK |
| 3332 | KGEAMCQSL |
| 3333 | KGEAMYQSL |
| 3334 | KGEAVCQSL |
| 3335 | KGESMCQSL |
| 3336 | KGGAMCQSL |
| 3337 | KSDPNRWAG |
| 3338 | KSFAGTTHM |
| 3339 | KSVAGTTHM |
| 3340 | KVEAMCQSL |
| 3341 | LEWYPNHHN |
| 3342 | LEWYPNHLN |
| 3343 | LEWYPNHRN |
| 3344 | LEWYPNHSN |
| 3345 | LEWYPYHRN |
| 3346 | LICFALNAP |
| 3347 | MVYCNRKSN |
| 3348 | QFHVPQSDS |
| 3349 | QFHVPQSGS |
| 3350 | QFRVPQSDS |
| 3351 | QNNNLYTQV |
| 3352 | SAGMLAQMC |
| 3353 | SAGMRAQMC |
| 3354 | SAGMRTQMC |
| 3355 | SAGMWAQMC |
| 3356 | SAGVRAQMC |
| 3357 | SAVMRAQMC |
| 3358 | SEQYVAYSG |
| 3359 | SKQYVAYSE |
| 3360 | SKQYVAYSG |
| 3361 | SKQYVTYSG |
| 3362 | TASKYIGRT |
| 3363 | TATEYIGRT |
| 3364 | TATKYIGRT |
| 3365 | TKPSMEVNC |
| 3366 | VEANSACQN |
| 3367 | VEANSECQN |
| 3368 | VEANSEFQN |
| 3369 | VEANSGCQN |
| 3370 | VICFALNAP |
| 3371 | VICFALNAS |
| 3372 | VICFALNTP |
| 3373 | VSLFCRQYS |
| 3374 | WCVHAYWQV |
| 3375 | WRVHAYWQV |
| 3376 | WRVHSYWQV |
| 3377 | YIHWYSKSS |
| 3378 | WGQSYNYWP |
| 3379 | SIHSIVKGE |
| 3380 | GMLEFMNMT |
| 3381 | QMYWVQNWE |
| 3382 | STDPHTSHH |
| 3383 | TLTEVNRLA |
| 3384 | SCASCRFPL |
| 3385 | ETHWDIDSN |
| 3386 | EAAHQTNIW |
| 3387 | MGTEWTQSY |
| 3388 | QQSFFFGEQ |
| 3389 | CVDTNRMWQ |
| 3390 | DVCKATTTE |
| 3391 | TTYPHNTHG |
| 3392 | KGLITENNY |
| 3393 | LHKAYVTDS |
| 3394 | KQATNNNGW |
| 3395 | CFARTLSYD |
| 3396 | MPYPLNVKE |
| 3397 | EQTHTWLCR |
| 3398 | ELKYVMMTA |
| 3399 | LARTTTNSE |
| 3400 | TLTTDKTYS |
| 3401 | TRSYFDQSC |
| 3402 | PYIMEFHPF |
| 3403 | KIRDCDGNV |
| 3404 | EIRQFVKGA |
| 3405 | APFFTFTNH |
| 3406 | TQCKQLTVM |
| 3407 | CQNTAVRAI |
| 3408 | KAVLFLKDC |
| 3409 | DVTQHTSCT |
| 3410 | VCQYCELGG |
| 3411 | AVVCKMNYH |
| 3412 | PCQQLMCFM |
| 3413 | TVNRAMREE |
| 3414 | EVLKFCKEW |
| 3415 | SMSHNGQCL |
| 3416 | TNMLAMRGD |
| 3417 | MLAFDPMGR |
| 3418 | ASFFLNPLE |
| 3419 | QKAIVAQND |
| 3420 | ISMRSECED |
| 3421 | ISWISHASP |
| 3422 | CVEQYVEQG |
| 3423 | STTAYHQDY |
| 3424 | GWDMLSVAK |
| 3425 | MITNNCNIS |
| 3426 | TCHPNMSVP |
| 3427 | MEHTMDFGV |
| 3428 | KEFQLQMEP |
| 3429 | TIVVIPIKS |
| 3430 | SSIYKEIQE |
| 3431 | GTRIYGKVS |
| 3432 | EFVLGCQGI |
| 3433 | QSGMLTTFA |
| 3434 | YVDHWTLGD |
| 3435 | SRYVNNGTV |
| 3436 | AAQYMGIMK |
| 3437 | AEDTPYTIY |
| 3438 | DCVLTMVDK |
| 3439 | MTIKMEQQY |
| 3440 | NSQLSMTFG |
| 3441 | NMPSLRTRI |
| 3442 | EIHMFGQSE |
| 3443 | MVMMREPSN |
| 3444 | MNANETHGA |
| 3445 | WTTSIAVDT |
| 3446 | NDVWQPHPN |
| 3447 | GLSMVLHFV |
| 3448 | RCADNPYFR |
| 3449 | AFLHGQMWQ |
| 3450 | NANYWFYYD |
| 3451 | AIYTTASFD |
| 3452 | EAGCKMWPT |
| 3453 | DARFDQWHH |
| 3454 | ATGTYNLQE |
| 3455 | TTTNQFMFQ |
| 3456 | VCRLDNGPQ |
| 3457 | EYACMLMTQ |
| 3458 | YDYWMQAPT |
| 3459 | SHAQPIASH |
| 3460 | DFNGSNWLP |
| 3461 | RVNGSTCRH |
| 3462 | HIHMSDRGA |
| 3463 | NAYYDSHCE |
| 3464 | THVKRFDYE |
| 3465 | KKHKGWCRA |
| 3466 | MPWLDKPPC |
| 3467 | RRTDHGEPI |
| 3468 | GNECFHNVE |
| 3469 | YPTMTFPEE |
| 3470 | KVNCRMIIM |
| 3471 | WPNIKHQPI |
| 3472 | QCTAHNTCM |
| 3473 | DMTLQSVSS |
| 3474 | YFPSIQCYE |
| 3475 | RYPCCQSPF |
| 3476 | WKATPGGQC |
| 3477 | GHLFNNNEW |
| 3478 | MNSFYRAEW |
| 3479 | QWFTKGVGE |
| 3480 | INYISFVDR |
| 3481 | KMAGIGCWY |
| 3482 | LEIGGKTRT |
| 3483 | NGVGRSGDN |
| 3484 | AGMYKCQED |
| 3485 | YSDPKNMSI |
| 3486 | DVKSWAVCD |
| 3487 | TVNWYSGFG |
| 3488 | AAAGFSAPS |
| 3489 | AAARDRRQT |
| 3490 | AACAQVYHS |
| 3491 | AACGVLWSE |
| 3492 | AAEREKAHG |
| 3493 | AAGFVTCLD |
| 3494 | AAGHHANLG |
| 3495 | AAGYARTYD |
| 3496 | AANLNEGAK |
| 3497 | AANPHMHMR |
| 3498 | AASHLIVHE |
| 3499 | AATITYAPS |
| 3500 | AATMAPWMA |
| 3501 | AAVKKLSDD |
| 3502 | AAYLPSLVH |
| 3503 | ACCRMTTQQ |
| 3504 | ACDFIGQNE |
| 3505 | ACELQEMAM |
| 3506 | ACGQAIHHS |
| 3507 | ACIFRHQFY |
| 3508 | ACLEVYCQD |
| 3509 | ACLSSPIFE |
| 3510 | ACMTVYRSC |
| 3511 | ACQPSPSYT |
| 3512 | ACTQRQEEM |
| 3513 | ACYCYFMFI |
| 3514 | ACYRQHATK |
| 3515 | ADIWIQFDA |
| 3516 | ADRLINSSK |
| 3517 | ADYQTQTYN |
| 3518 | AECLSTKWV |
| 3519 | AEQWKLGSI |
| 3520 | AERDCEKGP |
| 3521 | AERMFHQHF |
| 3522 | AFCSMNATS |
| 3523 | AFIEQVMNC |
| 3524 | AFMAIMWTL |
| 3525 | AFQQPHKAN |
| 3526 | AGDRYTSED |
| 3527 | AGISLWCHN |
| 3528 | AGLTMQKCE |
| 3529 | AGSSCNTFH |
| 3530 | AHAEFAVDP |
| 3531 | AHAQYQSVA |
| 3532 | AHMPREWMA |
| 3533 | AHTAIGSCP |
| 3534 | AHVCLSGQW |
| 3535 | AHVDYNTCD |
| 3536 | AHVGVELAE |
| 3537 | AHYHVHSHG |
| 3538 | AIAHGSDYE |
| 3539 | AIDHRTMCY |
| 3540 | AIEKQPNFE |
| 3541 | AIESSMHCC |
| 3542 | AIGKSNQGL |
| 3543 | AIIHQSTAW |
| 3544 | AILDQNSLQ |
| 3545 | AINTIPMAQ |
| 3546 | AISQIMSNE |
| 3547 | AITAHLAHD |
| 3548 | AITQMEWIN |
| 3549 | AIVPESAAQ |
| 3550 | AKANRCEAE |
| 3551 | AKMPQCAWA |
| 3552 | AKMQNMACA |
| 3553 | AKNLTMLCR |
| 3554 | AKSYANDWA |
| 3555 | ALHHFVKAQ |
| 3556 | ALLMQTFAH |
| 3557 | ALMVFKSMW |
| 3558 | ALMVNQGNA |
| 3559 | ALNLSWWPM |
| 3560 | ALTAITTSS |
| 3561 | ALTNHMICP |
| 3562 | ALTYRDSMY |
| 3563 | AMLKYAYPG |
| 3564 | AMSWTQKGG |
| 3565 | AMVGYHCRE |
| 3566 | ANFPVNTLE |
| 3567 | ANGSVNCEE |
| 3568 | ANGTLAECG |
| 3569 | ANMYSNVTS |
| 3570 | ANTSGWTSG |
| 3571 | APDQANVYE |
| 3572 | APMSYSDMD |
| 3573 | APRVHSGNC |
| 3574 | APSPKAVYF |
| 3575 | AQEFIPSHP |
| 3576 | AQGTQCANL |
| 3577 | AQKQADINA |
| 3578 | AQRMAKIWA |
| 3579 | ARELLHWYG |
| 3580 | ARMPVSCLP |
| 3581 | ARSRTLSDG |
| 3582 | ARTECHHCG |
| 3583 | ARYKLMPHH |
| 3584 | ASCRLAQCC |
| 3585 | ASEGAGSWE |
| 3586 | ASEPITFVS |
| 3587 | ASFGYMIDD |
| 3588 | ASGIIKSNG |
| 3589 | ASGPIGTAA |
| 3590 | ASHGDGVGT |
| 3591 | ASKPVELGH |
| 3592 | ASLMKPEAV |
| 3593 | ASMAYCAQE |
| 3594 | ASMKRQENL |
| 3595 | ASMTDFVGV |
| 3596 | ASMWQHFVL |
| 3597 | ASQGVWQSH |
| 3598 | ASSTAWQWP |
| 3599 | ASTLCNRGQ |
| 3600 | ASTWQGHTD |
| 3601 | ASVESRIRI |
| 3602 | ATAAENIYE |
| 3603 | ATAVTANAN |
| 3604 | ATCKPDSCD |
| 3605 | ATDQFGHHD |
| 3606 | ATGKMMFPS |
| 3607 | ATHCINRAH |
| 3608 | ATIGRSCWD |
| 3609 | ATMPWDGNE |
| 3610 | ATMQESDRI |
| 3611 | ATSPSEHTN |
| 3612 | ATSSQVATI |
| 3613 | ATTFTRHNV |
| 3614 | ATTIWHNHM |
| 3615 | ATVFMQETT |
| 3616 | ATVHMERDC |
| 3617 | ATVMTVHQG |
| 3618 | ATYNPGVNW |
| 3619 | AVCSMHGTI |
| 3620 | AVDIIAWDT |
| 3621 | AVEEMVAKG |
| 3622 | AVEKCMTLH |
| 3623 | AVEPHCFGH |
| 3624 | AVGCYTMSG |
| 3625 | AVKMFIDRN |
| 3626 | AVMAQKIAD |
| 3627 | AVQKEYQYL |
| 3628 | AVSQFQCSQ |
| 3629 | AVVEHAVNR |
| 3630 | AVVRSVTRD |
| 3631 | AWDSKSNWD |
| 3632 | AWEMYHVID |
| 3633 | AWGTMNHWL |
| 3634 | AWHMYEMSE |
| 3635 | AWTEYSLSC |
| 3636 | AWYHHCHYD |
| 3637 | AYAFVGRDN |
| 3638 | AYDRMGGTY |
| 3639 | AYEQMPESC |
| 3640 | AYHNVKEWN |
| 3641 | AYSSTLHAE |
| 3642 | AYYEPRGWN |
| 3643 | CAAQEQRSR |
| 3644 | CAAWRQTQE |
| 3645 | CAAWYYGRT |
| 3646 | CAFRTAQMA |
| 3647 | CALIKHQQP |
| 3648 | CAMFRNVAQ |
| 3649 | CAMKGNVAQ |
| 3650 | CAMLKMKYD |
| 3651 | CAMPFRQSV |
| 3652 | CAMSRMVFS |
| 3653 | CAMWTSASP |
| 3654 | CAMYYHRAC |
| 3655 | CAQWIKEQQ |
| 3656 | CASAFVSYQ |
| 3657 | CAVGRHMAL |
| 3658 | CAYAYANLC |
| 3659 | CCEFKMWRG |
| 3660 | CCMWKNSWE |
| 3661 | CCNSRNVFP |
| 3662 | CCTKLQVLA |
| 3663 | CCTLKRCYD |
| 3664 | CDGQLDFHW |
| 3665 | CFFRVRLCI |
| 3666 | CFHIRNIDR |
| 3667 | CFHPTDQGW |
| 3668 | CFKYTFMQH |
| 3669 | CGAKMSRAA |
| 3670 | CGTIVHWST |
| 3671 | CGTTIAYCP |
| 3672 | CHFTQHKSD |
| 3673 | CHHWFNHAT |
| 3674 | CHLTTNVNE |
| 3675 | CIAIITRGP |
| 3676 | CIARKMVDD |
| 3677 | CIFPRNMAT |
| 3678 | CIGSIPMAA |
| 3679 | CIHAIPRER |
| 3680 | CIHARMVGM |
| 3681 | CIHDHKECV |
| 3682 | CINWTRKAP |
| 3683 | CIRLLCRDK |
| 3684 | CIRYEMGAA |
| 3685 | CITQRQAMW |
| 3686 | CIVLRSMGQ |
| 3687 | CIVVYSRSQ |
| 3688 | CIWKHSHFV |
| 3689 | CKAHYHDNF |
| 3690 | CKDAMNTGG |
| 3691 | CKFLLNQAR |
| 3692 | CKIASNEAF |
| 3693 | CKMEFMRVE |
| 3694 | CLCLTWAFG |
| 3695 | CLGYEKQCP |
| 3696 | CLHYTIVNN |
| 3697 | CLMEVQWGT |
| 3698 | CMDEVHMCT |
| 3699 | CMMPYSRTN |
| 3700 | CMYQSRAAY |
| 3701 | CNTPYWQQA |
| 3702 | CPFLMNRAF |
| 3703 | CQQRYNVGF |
| 3704 | CSAYYMKCS |
| 3705 | CSCWYQVQF |
| 3706 | CSDNLYCAM |
| 3707 | CSECWCKYW |
| 3708 | CSHDFTQYQ |
| 3709 | CSIQWWQWG |
| 3710 | CSKLHDHIN |
| 3711 | CSLQQMSIR |
| 3712 | CSNMYVASE |
| 3713 | CSQPMSMPN |
| 3714 | CSQSFQWSK |
| 3715 | CSSMWFLWG |
| 3716 | CSSPMSLQD |
| 3717 | CSTSRMASY |
| 3718 | CSVNRHHYW |
| 3719 | CSYELITNG |
| 3720 | CTFHTMRGH |
| 3721 | CTFIRHAMQ |
| 3722 | CTFLRSTFA |
| 3723 | CTFNQIRGP |
| 3724 | CTFQPNRVC |
| 3725 | CTGFMGWCM |
| 3726 | CTHPNALSP |
| 3727 | CTIAIRDSS |
| 3728 | CTIMAMQSH |
| 3729 | CTIRKQQMT |
| 3730 | CTKFEHRSL |
| 3731 | CTKHMLQGE |
| 3732 | CTMHMDAKY |
| 3733 | CTMWCYGCP |
| 3734 | CTNVRMKYE |
| 3735 | CTRQTTFHG |
| 3736 | CTVSKNYGQ |
| 3737 | CTYQYQAGM |
| 3738 | CTYTPLRAP |
| 3739 | CVCYHNSSQ |
| 3740 | CVDQHHLTP |
| 3741 | CVFAFGMKH |
| 3742 | CVFAHNRGF |
| 3743 | CVFMDRMAY |
| 3744 | CVFPRHHMF |
| 3745 | CVGLVMRPQ |
| 3746 | CVGPFGSMM |
| 3747 | CVHCYNRGY |
| 3748 | CVHIRNIGW |
| 3749 | CVHTVIRGW |
| 3750 | CVIMIGELS |
| 3751 | CVIQRMHVY |
| 3752 | CVKWCNRCS |
| 3753 | CVLASRADR |
| 3754 | CVLMRWAWP |
| 3755 | CVMPRHRFP |
| 3756 | CVMSHLSGK |
| 3757 | CVMTYWEAH |
| 3758 | CVMVWAEKG |
| 3759 | CVNHARQMS |
| 3760 | CVNPTTTFG |
| 3761 | CVPSYYNNR |
| 3762 | CVSYMFSQC |
| 3763 | CVTSLYGQY |
| 3764 | CVVEYPWTW |
| 3765 | CVVGKAKLA |
| 3766 | CWCCWNNPG |
| 3767 | CWEWTMINC |
| 3768 | CWNDEPEKC |
| 3769 | CWNYVHRAQ |
| 3770 | CWRVDRNHS |
| 3771 | CWSNVHRGS |
| 3772 | CWSVRHAWQ |
| 3773 | CWWLWNARE |
| 3774 | CYCCLRCHW |
| 3775 | CYCTNEHPS |
| 3776 | CYPACICTQ |
| 3777 | CYVYRHVIC |
| 3778 | CYYLSIVVC |
| 3779 | DAASDMFDP |
| 3780 | DAGYFTLHA |
| 3781 | DAIRWNKMV |
| 3782 | DALMREAGH |
| 3783 | DAMYYNFIT |
| 3784 | DAQSSPTAF |
| 3785 | DARFKPMMC |
| 3786 | DASMYYECS |
| 3787 | DATHMMCMH |
| 3788 | DCEPREGYW |
| 3789 | DFGAEKASE |
| 3790 | DFHYNSDWW |
| 3791 | DFLIHGYSG |
| 3792 | DFMKMQQHG |
| 3793 | DFYTMAEHF |
| 3794 | DGFKMQFDR |
| 3795 | DGTDWDQFS |
| 3796 | DHSFHWAYD |
| 3797 | DHSHNHNFF |
| 3798 | DHVPISTYF |
| 3799 | DIERSSRSM |
| 3800 | DIKCTMTES |
| 3801 | DIMCGQEHK |
| 3802 | DITINSFGS |
| 3803 | DKCRKDWHF |
| 3804 | DKKFLECVI |
| 3805 | DKLLMPFDR |
| 3806 | DKLYVDVMA |
| 3807 | DKMIAMVAD |
| 3808 | DKNDKWHAM |
| 3809 | DKYLCYTTP |
| 3810 | DLYVSQKAH |
| 3811 | DMAQPQAAE |
| 3812 | DMEWEHCHL |
| 3813 | DMFMIHAVQ |
| 3814 | DMHMWVHGM |
| 3815 | DMMYVRKDA |
| 3816 | DMYQFPMHG |
| 3817 | DNQMKNEMR |
| 3818 | DNRCIIGHN |
| 3819 | DNSTQIIGE |
| 3820 | DPCWIWKTI |
| 3821 | DPGRFQIGN |
| 3822 | DPPKEPKEG |
| 3823 | DPRDYDHHS |
| 3824 | DPTGEGDII |
| 3825 | DQGVMRQNE |
| 3826 | DQMGHTWQG |
| 3827 | DQMIQVEFT |
| 3828 | DQQPYVSGL |
| 3829 | DQRYGTSFS |
| 3830 | DRCYFGMGH |
| 3831 | DRNWYRHPG |
| 3832 | DRRPVDCMN |
| 3833 | DSASIPNYE |
| 3834 | DSFRKGTFA |
| 3835 | DSFTYTHGW |
| 3836 | DSIFPPHTI |
| 3837 | DSMLNGMGW |
| 3838 | DSQAAPTCP |
| 3839 | DSRTVFVDN |
| 3840 | DSVDWFTSG |
| 3841 | DTFGVACYL |
| 3842 | DTGMDAKYP |
| 3843 | DTKSYQAPA |
| 3844 | DTKWLHWMH |
| 3845 | DTTNRNMYS |
| 3846 | DVAEAWKHA |
| 3847 | DVAWHHWTF |
| 3848 | DVDRGRSMS |
| 3849 | DVDRIMPQP |
| 3850 | DVDWTYMTL |
| 3851 | DVHFKMTMA |
| 3852 | DVLCLHNHF |
| 3853 | DVLFTDVGF |
| 3854 | DVTFRWGIC |
| 3855 | DVTLMENAR |
| 3856 | DVWFNHTVS |
| 3857 | DVYPPQGLM |
| 3858 | DVYQLHAMQ |
| 3859 | DWAKSDRIT |
| 3860 | DWFIQYHRG |
| 3861 | DWSWYFQGS |
| 3862 | DYAAISSMA |
| 3863 | DYAKQDSSC |
| 3864 | DYNLAGLLP |
| 3865 | DYRQWDQQY |
| 3866 | DYVQVNQQP |
| 3867 | EAASADCQD |
| 3868 | EAAYGCKIG |
| 3869 | EADKYPYCP |
| 3870 | EADWNIGDT |
| 3871 | EAERIVRGP |
| 3872 | EAFCLHGWA |
| 3873 | EAGFWTQQA |
| 3874 | EAGSFQHCA |
| 3875 | EAHNVFDNP |
| 3876 | EAHPHWWVT |
| 3877 | EANDSLKAN |
| 3878 | EANLSNVRA |
| 3879 | ECFPSIQNL |
| 3880 | ECGLSHDCG |
| 3881 | ECHVRDSHL |
| 3882 | ECIKQSECA |
| 3883 | ECIQVVRGS |
| 3884 | ECKDVEDIF |
| 3885 | ECMLCPEAS |
| 3886 | ECQTSPVAY |
| 3887 | ECRAGDPVN |
| 3888 | ECSLKDHHS |
| 3889 | ECTANTHYY |
| 3890 | ECWMQNEHA |
| 3891 | EDCALHQSE |
| 3892 | EDHWLFWKQ |
| 3893 | EETILIDCH |
| 3894 | EEVMLGCLQ |
| 3895 | EFAADAHGM |
| 3896 | EFAGITDMG |
| 3897 | EFCEYAQAT |
| 3898 | EFHQQVNTW |
| 3899 | EFMHAYEAT |
| 3900 | EFSECHAFD |
| 3901 | EFSNPCSFG |
| 3902 | EFVKAFMQI |
| 3903 | EGHGAVLNP |
| 3904 | EGKWIGLQT |
| 3905 | EGMKSWNST |
| 3906 | EGRSSENND |
| 3907 | EGSPYFMAK |
| 3908 | EHALEHTQL |
| 3909 | EHELLKQYE |
| 3910 | EHGMILRLG |
| 3911 | EHLFLIHEA |
| 3912 | EHRQKMAST |
| 3913 | EHRTAFFYS |
| 3914 | EHVFEEVHS |
| 3915 | EIDNVCRQA |
| 3916 | EIGCAHAEE |
| 3917 | EIGRNCALF |
| 3918 | EILLSMMEQ |
| 3919 | EILQPQVMI |
| 3920 | EIMCKGTMG |
| 3921 | EIQQVGRDF |
| 3922 | EIRHWGWLV |
| 3923 | EISNVRCAP |
| 3924 | EIVPTMHCK |
| 3925 | EIVYQHISQ |
| 3926 | EKHNFTQMV |
| 3927 | EKKFNHQIN |
| 3928 | EKLYSVSVA |
| 3929 | EKRLESAGW |
| 3930 | EKSNLFTVR |
| 3931 | EKYMYHDVQ |
| 3932 | ELDFIIMPG |
| 3933 | ELNGAWWKA |
| 3934 | EMGAVGSFK |
| 3935 | EMHRNELAM |
| 3936 | EMMVKPSYC |
| 3937 | EMRIQTAQQ |
| 3938 | EMVLCVTHE |
| 3939 | ENDQIAHEQ |
| 3940 | ENGYMHQYL |
| 3941 | ENIKTPFGT |
| 3942 | ENTYQDTIP |
| 3943 | EPKDVSHMT |
| 3944 | EPQSFNHWS |
| 3945 | EQARIDTIG |
| 3946 | EQDQTKTMH |
| 3947 | EQELPVTLK |
| 3948 | EQPQKPSAW |
| 3949 | EQTKVMHCC |
| 3950 | EQVSKGVYA |
| 3951 | ERGPTDNLH |
| 3952 | ERHESHQEF |
| 3953 | ERINKFNAD |
| 3954 | ERRSAESLQ |
| 3955 | ESAQSEGEF |
| 3956 | ESDIGHPYK |
| 3957 | ESDIMRSPM |
| 3958 | ESEQSQDNH |
| 3959 | ESGPKWRFP |
| 3960 | ESNGFVNGV |
| 3961 | ESSRTGSHA |
| 3962 | ESTESHFCE |
| 3963 | ESTQMCTCC |
| 3964 | ESVKFGKMP |
| 3965 | ESYTLYQHL |
| 3966 | ETACNHTAW |
| 3967 | ETASQQHDA |
| 3968 | ETCNRGRLF |
| 3969 | ETFAMNAAW |
| 3970 | ETHFYEVKF |
| 3971 | ETLAKGYDW |
| 3972 | ETLRMFGVY |
| 3973 | ETMPNKLQY |
| 3974 | ETNIDNRAM |
| 3975 | ETRINIEAH |
| 3976 | ETTNAAWFR |
| 3977 | ETVSGMHER |
| 3978 | EVCFDVCGE |
| 3979 | EVCMAVVDN |
| 3980 | EVDHQFHFC |
| 3981 | EVGPGSHCE |
| 3982 | EVNHMMCDM |
| 3983 | EVQKCMFVE |
| 3984 | EVRWIPWHY |
| 3985 | EVSLFSCEF |
| 3986 | EVYITGHEI |
| 3987 | EWKFCWDAI |
| 3988 | EWQHVGATC |
| 3989 | EYCRSNFQH |
| 3990 | EYESCICAS |
| 3991 | EYLRFIAHD |
| 3992 | FACMRQMAY |
| 3993 | FATGKHHYL |
| 3994 | FATHTYDYW |
| 3995 | FAVPSKVTM |
| 3996 | FCYRMTDDR |
| 3997 | FDMEGFREL |
| 3998 | FERTVHTDK |
| 3999 | FGDWKHGDG |
| 4000 | FGPGSHGNN |
| 4001 | FKGPYHYQS |
| 4002 | FLFYLNMHE |
| 4003 | FLMRRNGAD |
| 4004 | FLTERCQTE |
| 4005 | FMNHFMMWT |
| 4006 | FMQKECVEE |
| 4007 | FNADIAHCD |
| 4008 | FNMLVLETW |
| 4009 | FNSSFMGQA |
| 4010 | FPHKWNAMS |
| 4011 | FPVDMQWMY |
| 4012 | FQGPIAPME |
| 4013 | FSAMFRRHC |
| 4014 | FSGNLSTYA |
| 4015 | FSKLTGQDF |
| 4016 | FSLKTESRI |
| 4017 | FTDDDVNRF |
| 4018 | FTGLCDIWQ |
| 4019 | FTRALLDMG |
| 4020 | FVRTKEDLV |
| 4021 | GADDKGTHC |
| 4022 | GADIYIETS |
| 4023 | GADKLFMYG |
| 4024 | GADNVQKIC |
| 4025 | GADPFEGQN |
| 4026 | GAEIEQLAF |
| 4027 | GAEYDQRVW |
| 4028 | GAHFIMSSL |
| 4029 | GAQFCTQAR |
| 4030 | GARHNGIFT |
| 4031 | GARQFNCLE |
| 4032 | GASKLSHER |
| 4033 | GASRYQWGN |
| 4034 | GATDCGSCP |
| 4035 | GAVECSEYK |
| 4036 | GAVPHPVCA |
| 4037 | GAVQKLIQC |
| 4038 | GAVSANWQS |
| 4039 | GCASIGIHE |
| 4040 | GCDWLMCEL |
| 4041 | GCERMERVW |
| 4042 | GCFFSQRGY |
| 4043 | GCFYTQRAY |
| 4044 | GCGKLGVDR |
| 4045 | GCHQLMNFG |
| 4046 | GCIRSISWP |
| 4047 | GCIRVDSGF |
| 4048 | GCMHYVLTE |
| 4049 | GCMQSIHMQ |
| 4050 | GCRFVILDH |
| 4051 | GCTKYGRHN |
| 4052 | GCVVKNTHS |
| 4053 | GDKWISIQD |
| 4054 | GEMCNVSGN |
| 4055 | GFDQRGMEG |
| 4056 | GGTEIAMCF |
| 4057 | GHAMFNSRD |
| 4058 | GHFQTMMAQ |
| 4059 | GHITCATMS |
| 4060 | GHKQCFHQT |
| 4061 | GHNCCHMIE |
| 4062 | GHVQESWMW |
| 4063 | GIAQKNRNP |
| 4064 | GIEHLQNTS |
| 4065 | GIMCSGMCP |
| 4066 | GIMSVMGIG |
| 4067 | GINHRHVSA |
| 4068 | GKKDQVKGG |
| 4069 | GKTELHVEV |
| 4070 | GLGFKHSMG |
| 4071 | GLVEIHYTE |
| 4072 | GLYPHQAMF |
| 4073 | GMAVINEVE |
| 4074 | GMGKYNQWW |
| 4075 | GMGMVEIVG |
| 4076 | GMWVNPMAL |
| 4077 | GNQHWDVCG |
| 4078 | GQCYCYVQP |
| 4079 | GQSGLHVCY |
| 4080 | GRIQRMESD |
| 4081 | GSAQEPIWA |
| 4082 | GSILSIGFH |
| 4083 | GSSWIDQQG |
| 4084 | GSTRCVKWV |
| 4085 | GTEHHPFFD |
| 4086 | GTERLPAHQ |
| 4087 | GTEYERHTF |
| 4088 | GTFWRDSQL |
| 4089 | GTGKHAWCY |
| 4090 | GTHYDASAG |
| 4091 | GTIRLDTQY |
| 4092 | GTLEKRAIF |
| 4093 | GTLSYMMWW |
| 4094 | GTSTIRAEH |
| 4095 | GTYIELACF |
| 4096 | GVCFKLTDN |
| 4097 | GVFQCPQSV |
| 4098 | GVHQKNCPE |
| 4099 | GVKAREIEI |
| 4100 | GVNDLRHVA |
| 4101 | GVNHRMDHF |
| 4102 | GVRVFMSDV |
| 4103 | GVSMCYNWN |
| 4104 | GVSSTHQHR |
| 4105 | GVVANLNST |
| 4106 | GVVKMENMA |
| 4107 | GVYTTEQCV |
| 4108 | GWEYHPAFG |
| 4109 | GWTNYALMT |
| 4110 | GYHWTNTAS |
| 4111 | GYLPLEIIA |
| 4112 | GYMPCMCQD |
| 4113 | GYVQDFHGP |
| 4114 | HAEQALSVA |
| 4115 | HAGSCTLND |
| 4116 | HAGSWQYGP |
| 4117 | HAMPFCGRS |

TABLE 73
Sequences of the 581 to 589 Region in
AAV5 VP1 Capsid Polypeptide that Drive Bone
Marrow Tissue Tropism

| | 581-589 |
|---|---|
| SEQ ID NO | Sequence |
| 6118 | DVDVVFRWV |
| 6119 | GNMHNFSAW |
| 6120 | QVVKFTGYH |
| 6121 | VQMNLDAAP |
| 6122 | YSFATGNAA |
| 6123 | MVDPYGCEG |
| 6124 | CEMQVQLCV |
| 6125 | AVDHYSNGA |
| 6126 | AAQTLECHK |
| 6127 | ADHLACALQ |
| 6128 | ADSDVHSEP |
| 6129 | AHENYRYHQ |
| 6130 | AHLNCDKSM |
| 6131 | AHRCGCFKC |
| 6132 | AILKMTKVL |
| 6133 | AKTGGEPDS |
| 6134 | AKVNMMKQF |
| 6135 | ANNTYPVCA |
| 6136 | AQWISPWPT |
| 6137 | ASKFEIACF |
| 6138 | ATCLIVCEA |
| 6139 | ATNNADCGV |
| 6140 | CARANCVLS |
| 6141 | CFDQPLYID |
| 6142 | CMRHGDRCA |
| 6143 | CRIDHMETY |
| 6144 | CVMEDNEAP |
| 6145 | CWEEGMPFY |
| 6146 | CWILHHRCG |
| 6147 | CYEHDHGQA |
| 6148 | CYNDNWVDV |
| 6149 | DAHKSMKSQ |
| 6150 | DCLHCWDFC |
| 6151 | DENWRDTCG |
| 6152 | DFLEEHSHM |
| 6153 | DFNFLRQQA |
| 6154 | DFTQLRMSG |
| 6155 | DGSKNQHSY |
| 6156 | DIAQLILTT |
| 6157 | DIGRNVGEC |
| 6158 | DILGKGEPC |
| 6159 | DIMSYATGI |
| 6160 | DKNYPTHKQ |
| 6161 | DLGCVQENF |
| 6162 | DQECNEIGF |
| 6163 | DTDCHSYMD |
| 6164 | DTNDYTSCD |
| 6165 | DVIRSESRR |
| 6166 | DVSYHKKMH |
| 6167 | DVVSMKWGV |
| 6168 | ELASSWLVI |
| 6169 | ELLEFNAMQ |
| 6170 | ENAYKTYPL |
| 6171 | ERNRLHHRY |
| 6172 | ESHALHMCN |
| 6173 | ESSYWDKPK |
| 6174 | EVVRQKVIC |
| 6175 | EYSWSAQYN |
| 6176 | FAAMCNKWD |
| 6177 | FCSTYNAGT |
| 6178 | FEQSEHKPT |
| 6179 | FSHAHNWMT |
| 6180 | FSHRFDAIF |
| 6181 | FSTPARINN |
| 6182 | FTAQLAVWM |
| 6183 | GDMCEDMCT |
| 6184 | GHDFQKHAE |
| 6185 | GLHQFSICM |
| 6186 | GPAVPEQYG |
| 6187 | GRMLVDRHA |
| 6188 | GTGPMHHKW |
| 6189 | GYDPPEGMV |
| 6190 | GYYSMHHEI |
| 6191 | HATTVEFCI |
| 6192 | HESKTAHRP |
| 6193 | HFHQQRKTY |
| 6194 | HGEPFCMAP |
| 6195 | HHQQCKLDW |
| 6196 | HKMECAQSM |
| 6197 | HPHQIPIDI |
| 6198 | HRPEQLHEG |
| 6199 | HSWMRKSQC |
| 6200 | HWCIMKGMR |
| 6201 | ICEYFRVQA |
| 6202 | ICEYRYCIF |
| 6203 | IDAWGHFSE |
| 6204 | IGYSTWPRE |
| 6205 | ILNTCLVDQ |
| 6206 | IMDVRVKRS |
| 6207 | IMVMINTPY |
| 6208 | INTNYPCLN |
| 6209 | IPCILELVV |
| 6210 | IPVMPCNCE |
| 6211 | IWILRARGD |
| 6212 | KDDTQSHMF |
| 6213 | KEQRPPNAG |
| 6214 | KFVQYQHYV |
| 6215 | KGRWIFEYA |
| 6216 | KHDGEIRVL |
| 6217 | KHIVGQDGG |
| 6218 | KIYLNQILR |
| 6219 | KLQMGQDNS |
| 6220 | KLSDHEMMY |
| 6221 | KQNDTVKIP |
| 6222 | KTDRMAMMM |
| 6223 | LAFTMMWPW |
| 6224 | LATFWGHQD |
| 6225 | LCAPTQWYM |
| 6226 | LCGKAGLYH |
| 6227 | LDLCMLFLF |
| 6228 | LEMQRGMET |
| 6229 | LFDKDIARK |
| 6230 | LFKFHSGLQ |
| 6231 | LGLTEGQSM |
| 6232 | LNGWWDCTR |
| 6233 | LPCTSIMWY |
| 6234 | LPEHIQQMH |
| 6235 | LPENRNKRH |
| 6236 | LPKSKYMFQ |
| 6237 | LQANEWNMC |
| 6238 | LQQPIVHIE |
| 6239 | LVMCPPFFG |
| 6240 | LYDVVHRIW |
| 6241 | MGHYCHARR |
| 6242 | MHQFRCHYD |
| 6243 | MMAPLKVFD |
| 6244 | MNMEDRCTF |
| 6245 | MNNRWSDCD |
| 6246 | MTEPVLLDT |
| 6247 | MVQHIEQWE |
| 6248 | NCWLPDNPI |
| 6249 | NFKQHDTFC |
| 6250 | NGFYWVFVM |
| 6251 | NLCPNTVDV |
| 6252 | NMGCPMMTE |
| 6253 | NNSHLYHDN |
| 6254 | NWDSPSNKS |
| 6255 | NYFPTHAAW |
| 6256 | PDQVDRNTP |
| 6257 | PEKAKMVWH |
| 6258 | PPADPATES |
| 6259 | PQTMMCKRW |
| 6260 | PSMLWSVVV |
| 6261 | QEGNGFHNT |
| 6262 | QEKLWEQGS |
| 6263 | QEVRSEHYS |
| 6264 | QFGPGCFKP |
| 6265 | QQIWLCQEI |
| 6266 | QQLCVDESH |
| 6267 | QRPTCKNTA |
| 6268 | QSMVSCYEG |
| 6269 | QTRKNEIGI |
| 6270 | QWMLRKEVQ |
| 6271 | QWTACQATN |
| 6272 | QYMLAEDWR |
| 6273 | RADGLATVV |
| 6274 | RCNMMGKGA |
| 6275 | RDRYCDPHQ |
| 6276 | RDRYCDPLQ |
| 6277 | RKFWLAGKF |
| 6278 | RLQIIMYRV |
| 6279 | RMKGAKRWY |
| 6280 | RNEYQSACW |
| 6281 | RRNMPIPSW |
| 6282 | RSMYANNDV |
| 6283 | RYNNCPNLW |
| 6284 | SAMSRAVYY |
| 6285 | SEKTQEVCL |
| 6286 | SGEGITKMY |
| 6287 | SILITPYAN |
| 6288 | SLCWEVDLM |
| 6289 | SLYITPYHT |
| 6290 | SMYTNCTFF |
| 6291 | SQKQICGDT |
| 6292 | SWLPENPVH |
| 6293 | SYAYSHSAT |
| 6294 | TNVYQNVEA |
| 6295 | TPPCCVIQF |
| 6296 | TQEACHHDP |
| 6297 | TRSYFDQSC |
| 6298 | TSNTFQRHG |
| 6299 | TVKYHQDQD |
| 6300 | TVNNDPNGI |
| 6301 | VCHAFTCNL |
| 6302 | VCTIRAECP |
| 6303 | VDTIRFPRM |
| 6304 | VEKTHNKWM |
| 6305 | VESMNHASM |
| 6306 | VGICRLKAV |
| 6307 | VHYYACHRF |
| 6308 | VKEPATYKY |
| 6309 | VNAGEVYRS |
| 6310 | VPSHMPHHP |
| 6311 | VTDAAMFMG |
| 6312 | VTNPLEGRI |
| 6313 | VVEMNGDDD |
| 6314 | VVGVPARAQ |
| 6315 | VVQYRLDPP |
| 6316 | VYTSTQPQE |
| 6317 | WTHTVTSDW |
| 6318 | WWDHLPELM |
| 6319 | YAFNTPWMT |
| 6320 | YVEFEDQNG |
| 6321 | YVYDAQRQD |
| 6322 | YWRYHYYYM |
| 6323 | QNKAFLDAV |
| 6324 | FDHNAKVLD |
| 6325 | YNNIMQIMV |
| 6326 | FWLGLFPAK |
| 6327 | NNDHYDSES |
| 6328 | MCQNVEKEY |
| 6329 | QMDWVAECR |
| 6330 | MSSRKEFLT |
| 6331 | SCRMVYGRA |
| 6332 | AVHKQTAAW |
| 6333 | DCAWMQKEW |
| 6334 | QKNVSTKYD |
| 6335 | DYVDQCQVS |
| 6336 | TAVLYETEF |
| 6337 | VPDYVPMNW |
| 6338 | TSRDGGWAY |
| 6339 | LPLPMQSMC |
| 6340 | DGGRANTLH |
| 6341 | QADNGTMRV |
| 6342 | MFSHGHVMF |
| 6343 | PYHNPWECR |
| 6344 | TTYPHNTHG |
| 6345 | EQTYYKITW |
| 6346 | TSLCAKPHF |
| 6347 | FCHEVFSHT |
| 6348 | CKVDIHDYM |
| 6349 | ETSTHCCKV |
| 6350 | PAYRMECCE |
| 6351 | GMVELGSCW |
| 6352 | EAMTCTKNH |
| 6353 | DNVFSNSPN |
| 6354 | HILIMQGVS |
| 6355 | MHGIARMQQ |
| 6356 | HMDHCQCHE |
| 6357 | EKFWFKTMN |
| 6358 | QLDTYMEGR |
| 6359 | NMKPKDVFR |
| 6360 | QPGVRTDDY |
| 6361 | WCNWQFAQH |
| 6362 | GILVQGGSM |
| 6363 | ILKQASCKG |
| 6364 | AIIQWACWA |
| 6365 | WPTMLSAHM |
| 6366 | DFNGSNWLP |
| 6367 | FTEPMCMQG |
| 6368 | KTDKAGLME |
| 6369 | KWEQRSKMS |
| 6370 | TLHYFHGYQ |
| 6371 | MNHQCMLWD |
| 6372 | IYCLCQDSD |
| 6373 | TLNSRHPEC |
| 6374 | ACTKQKTVQ |
| 6375 | KTCLCIFTK |
| 6376 | SSNFNHHID |
| 6377 | QFMMGQQTP |
| 6378 | IAVASHAHG |
| 6379 | KTGMGDLFL |
| 6380 | RITQGESGM |
| 6381 | STVGQNHCG |
| 6382 | KVNCRMIIM |
| 6383 | YVDHWTLGD |
| 6384 | TMPDRDPTY |
| 6385 | QKAIVAQND |
| 6386 | CMEVTCFVV |
| 6387 | LESVKMDCN |
| 6388 | VLKSNLTGM |
| 6389 | AWMCCARHD |
| 6390 | RNDNTCHAY |
| 6391 | MYDTMGAWC |
| 6392 | ILEAIVNFW |
| 6393 | AHAWLQHWA |
| 6394 | ATGLTDHQW |
| 6395 | DVKSWAVCD |
| 6396 | PDLWYEKSS |
| 6397 | SECEVLCSL |
| 6398 | RYPCCQSPF |
| 6399 | MNSFYRAEW |
| 6400 | RTRISQMFG |
| 6401 | RDVGEDNSA |
| 6402 | ATGTYNLQE |
| 6403 | DQEAQVVTG |
| 6404 | PMELWRTWY |
| 6405 | ASMHEETCL |
| 6406 | DMTLQSVSS |
| 6407 | EVAFTTSSD |
| 6408 | SPQQPSVFH |
| 6409 | KSTYYNLKW |
| 6410 | SQERMDYDG |
| 6411 | MNLQIGSKG |
| 6412 | EIHMFGQSE |
| 6413 | MPWLDKPPC |
| 6414 | QCTAHNTCM |
| 6415 | DQRHGNVSP |
| 6416 | EAGCKMWPT |
| 6417 | ATMNLSWGL |
| 6418 | QAKMILDGT |
| 6419 | INYISFVDR |
| 6420 | WKATPGGQC |
| 6421 | YCECGHWPN |
| 6422 | SNVACIQAF |
| 6423 | QSRCVDNTV |
| 6424 | YSDPKNMSI |
| 6425 | ASKQGTHDY |
| 6426 | NHSLCWDSK |
| 6427 | HSMMPGWPS |
| 6428 | EWMQNWISM |
| 6429 | AAADVEGTT |
| 6430 | AAAPWCEFR |
| 6431 | AAAWPEWTM |
| 6432 | AACNEDHHW |
| 6433 | AACSRHYES |
| 6434 | AAELYKCEN |
| 6435 | AAFEGEACF |
| 6436 | AAFEWMRAP |
| 6437 | AAGWRDGRR |
| 6438 | AAHCEYSTI |
| 6439 | AAKTFYWAR |
| 6440 | AALASDHDF |
| 6441 | AAQFLHIVK |
| 6442 | AASCGYHGW |
| 6443 | AASQAQSLT |
| 6444 | AAVCTQFSL |
| 6445 | AAVGSAQIE |
| 6446 | AAWDIELNE |
| 6447 | AAWVSGKLY |
| 6448 | ACALDGASN |
| 6449 | ACEFGSLNQ |
| 6450 | ACFHANNWH |
| 6451 | ACHVQLSDA |
| 6452 | ACKHEISHA |
| 6453 | ACLNMWEER |
| 6454 | ACPSAELCY |
| 6455 | ACTSSQVKG |
| 6456 | ACVFKCWWP |
| 6457 | ADCQPQSFN |
| 6458 | ADWLYMQSC |
| 6459 | ADYDMLTAY |
| 6460 | AEIMCSQDH |
| 6461 | AEIQNWFAK |
| 6462 | AEQHTLGMG |
| 6463 | AFDTMVCGT |
| 6464 | AFFTNMSPS |
| 6465 | AFGPYWMEK |
| 6466 | AFHLWKEDQ |
| 6467 | AFKSFDSHI |
| 6468 | AFRAICAHG |
| 6469 | AFWIEQMDY |
| 6470 | AGDLWEIGS |
| 6471 | AGEHSRSER |
| 6472 | AGFPFEKFA |
| 6473 | AGGHIHADH |
| 6474 | AGQFRKLPY |
| 6475 | AGWPTSREM |
| 6476 | AHMGQVWWF |
| 6477 | AHSQLVSDR |
| 6478 | AHVLNTFGN |
| 6479 | AIEFWDDAL |
| 6480 | AIFHACNSG |
| 6481 | AIFRSEITY |
| 6482 | AIGNLTGAQ |
| 6483 | AIQASMDAF |
| 6484 | AIRCESDAA |
| 6485 | AISVGEVTT |
| 6486 | AISVRHQHH |
| 6487 | AITDALLYR |
| 6488 | AITSASAKN |
| 6489 | AIVGWKTPP |
| 6490 | AIWIEEFFH |
| 6491 | AKAHVCTIP |
| 6492 | AKDQLNITR |
| 6493 | AKEVTTCNW |
| 6494 | AKHTGVTFG |
| 6495 | AKKMWHCDQ |
| 6496 | AKYGPWENN |
| 6497 | AKYQWQSYP |
| 6498 | ALAWKNYDK |
| 6499 | ALCTENAER |
| 6500 | ALGPLHQGD |
| 6501 | ALMPSMFLS |
| 6502 | ALQQNSLGE |
| 6503 | ALVYAHWWG |
| 6504 | ALYEADQFD |
| 6505 | ALYQIKWGF |
| 6506 | AMDVIIEGA |
| 6507 | AMEYLRLQH |
| 6508 | AMGTTEVQV |
| 6509 | AMHLQERMP |
| 6510 | AMHNREGAI |
| 6511 | AMIQCAMPP |
| 6512 | AMWWGQAMG |
| 6513 | ANAAIREHL |
| 6514 | ANAERCNLA |
| 6515 | ANCRKDPMP |
| 6516 | ANENCSKGS |
| 6517 | ANGPHTDCI |
| 6518 | ANILWYHAS |
| 6519 | ANKCLDMRD |
| 6520 | ANLMSAKME |
| 6521 | ANPLCELQQ |
| 6522 | APRLTEACL |
| 6523 | APSWRNHMC |
| 6524 | APVLITNQQ |
| 6525 | AQDHRHSDW |
| 6526 | AQDWSATQG |
| 6527 | AQEKRLYGQ |
| 6528 | AQFSHLHSY |
| 6529 | AQHSHHECY |
| 6530 | AQSMPYSRH |
| 6531 | AQWNQLYGS |
| 6532 | ARATWPRTA |
| 6533 | ARAVDGLNN |
| 6534 | ARFIREHEW |
| 6535 | ARLWIPGEP |
| 6536 | ARRMDDDWI |
| 6537 | ASCAFAEQY |
| 6538 | ASCTYYHKW |
| 6539 | ASEHYMEVF |
| 6540 | ASGIEGEQG |
| 6541 | ASGWIQAKT |
| 6542 | ASIEKAEMH |
| 6543 | ASMDHNLDD |
| 6544 | ASMTKHNNF |
| 6545 | ASNHRLEKY |
| 6546 | ASQPFNPWV |
| 6547 | ASSARSIFE |
| 6548 | ASTAPVADQ |
| 6549 | ASTNSVWHV |
| 6550 | ASVMQLLCP |
| 6551 | ASWLYDNNC |
| 6552 | ATAMPQVRY |
| 6553 | ATCCYTHFD |
| 6554 | ATDDQMIKG |
| 6555 | ATDHSHPWP |
| 6556 | ATDPCACNI |
| 6557 | ATDVVDECH |
| 6558 | ATELYMNGH |
| 6559 | ATERWLEGN |
| 6560 | ATHDAMVTN |
| 6561 | ATKCYEYTW |
| 6562 | ATMPQECGA |
| 6563 | ATNHSHPWP |
| 6564 | ATQFGMTED |
| 6565 | ATQSVWMLN |
| 6566 | ATQTDYNIP |
| 6567 | ATSLPAHYD |
| 6568 | ATTLHERTH |
| 6569 | ATTYWHRGG |
| 6570 | ATYYWQFVW |
| 6571 | AVELAPCTN |
| 6572 | AVFWTHQEE |
| 6573 | AVMPNHWQW |
| 6574 | AVMYPMRLH |
| 6575 | AVNLKQFKT |
| 6576 | AVNPAWKHD |
| 6577 | AVNWKQDGY |
| 6578 | AVRDHELNN |
| 6579 | AVSMACLNT |
| 6580 | AVVDWQALE |
| 6581 | AVVPLMESA |
| 6582 | AVYEALRCL |
| 6583 | AVYFVPCRP |
| 6584 | AWEMEMEYE |
| 6585 | AWNPSGFMG |
| 6586 | AWTRCIVCA |
| 6587 | AWVMSVMLA |
| 6588 | AWVNCISGS |
| 6589 | AYAETSTHP |
| 6590 | AYDNHYLAP |
| 6591 | AYQHHEGVV |
| 6592 | AYTESQHNL |
| 6593 | CAADCGKGW |
| 6594 | CADPQERCD |
| 6595 | CAQNLNLWL |
| 6596 | CASAVQEDC |
| 6597 | CASEHSCWH |
| 6598 | CATFQNDVL |
| 6599 | CCELLIENT |
| 6600 | CDGRRLSMH |
| 6601 | CDVQQPTMM |
| 6602 | CDYHFKNWG |
| 6603 | CEPLVEIHG |
| 6604 | CFDYMELSP |
| 6605 | CFEDCFYCH |
| 6606 | CFEPQCVIN |
| 6607 | CFVPDTEWG |
| 6608 | CGQQNFRIP |
| 6609 | CHADLFWEQ |
| 6610 | CHNTANHIQ |
| 6611 | CHSYFWMPD |
| 6612 | CIATQAFMY |
| 6613 | CIHQDAPPH |
| 6614 | CISQAVMPP |
| 6615 | CKERCGYAN |
| 6616 | CKLQKCYYM |
| 6617 | CLCYFCCDA |
| 6618 | CLHCPIRRR |
| 6619 | CLSACDLLV |
| 6620 | CMMMYNTCR |
| 6621 | CMMQCMDMM |
| 6622 | CMNPRSYYF |
| 6623 | CNAKQQAAW |
| 6624 | CNCASRTEE |
| 6625 | CNDLRNQWE |
| 6626 | CNNPTTRRW |
| 6627 | CNWSKSSIA |
| 6628 | CPGVLQSCD |
| 6629 | CPQQCIWYG |
| 6630 | CQVEKVENE |
| 6631 | CQVPAWYDP |
| 6632 | CRVNDVMQN |
| 6633 | CSCTTDTDP |
| 6634 | CSDWYVECK |
| 6635 | CSEVLEDNA |
| 6636 | CSLPHVVHY |
| 6637 | CSQRIHSHC |
| 6638 | CSSDYSTLS |
| 6639 | CTCQAKIFS |
| 6640 | CTDRFAIGA |
| 6641 | CTFPNVHEA |
| 6642 | CTHSAFHYV |
| 6643 | CTIWKQCGH |
| 6644 | CTMCYYQNN |
| 6645 | CTQMVIHQE |
| 6646 | CTTAIGPSG |
| 6647 | CTVILPKVG |
| 6648 | CTWFDCSQE |
| 6649 | CVDTHHANV |
| 6650 | CVHATFDYT |
| 6651 | CVTTDHCAW |
| 6652 | CWENTNVTC |
| 6653 | CWEVVNTFR |
| 6654 | CWFIKRACA |
| 6655 | CWYVWDLWP |
| 6656 | DACFVNQPN |
| 6657 | DACSHTWAD |
| 6658 | DAEDTPATV |
| 6659 | DAGKFRKYV |
| 6660 | DAHQENLNW |
| 6661 | DAKDMMMTC |
| 6662 | DALFNHAAV |
| 6663 | DALSYMQME |
| 6664 | DANEFILMH |
| 6665 | DANNTLQHS |
| 6666 | DANYYFVWQ |
| 6667 | DAWPNQQMW |
| 6668 | DCAQNWSGS |
| 6669 | DCEWMSTGA |
| 6670 | DCGFQTAHT |
| 6671 | DCHKWDTNQ |
| 6672 | DCKMHQAAM |
| 6673 | DCKWLALYP |
| 6674 | DCMQDPAYR |
| 6675 | DCMVMSQGH |
| 6676 | DCNFRHQDP |
| 6677 | DCPEWGVGC |
| 6678 | DCQMILGDY |
| 6679 | DCQPMEWWE |
| 6680 | DCRHFQSQE |
| 6681 | DCTEIVSGM |
| 6682 | DCTMYCKQS |
| 6683 | DCTPYQGGH |
| 6684 | DCYSKRFTP |
| 6685 | DCYYLKYSA |
| 6686 | DDEYNLTGL |
| 6687 | DDPNYAWEA |
| 6688 | DDQLYAWGM |
| 6689 | DDSLLFHMD |
| 6690 | DEASDLQYA |
| 6691 | DEHPHPEHC |
| 6692 | DEIQIGGSF |
| 6693 | DEMTFEQLF |
| 6694 | DEQHICMQT |
| 6695 | DESEMTSGG |
| 6696 | DEWWWSREE |
| 6697 | DFASRCNYV |
| 6698 | DFCFNESYW |
| 6699 | DFHCYSTER |
| 6700 | DFMTLVTHQ |
| 6701 | DFQAYSWNH |
| 6702 | DFRHVDKHA |
| 6703 | DFTQGMCTP |
| 6704 | DGDAQSCAY |
| 6705 | DGDCLQRHL |
| 6706 | DGFNYELQP |
| 6707 | DGGHMMNQD |
| 6708 | DGHNFDGHN |
| 6709 | DGIWTHKAD |
| 6710 | DGQFIFSQN |
| 6711 | DGYNMLWDP |
| 6712 | DHCEMHDCW |
| 6713 | DHFHMELTK |
| 6714 | DHQSYYTQK |
| 6715 | DIEFYCAYP |
| 6716 | DIEMFGSEM |
| 6717 | DIEYQSMGS |
| 6718 | DIGWLAPTA |
| 6719 | DIICDCDMF |
| 6720 | DILLDSRHS |
| 6721 | DITLIMSVH |
| 6722 | DKGQLCSLQ |
| 6723 | DKIWIWQAS |
| 6724 | DKLARSVSC |
| 6725 | DKMELNAHW |
| 6726 | DKQQAKDQL |
| 6727 | DKQRPMCVC |
| 6728 | DKQWFWFGH |
| 6729 | DKTSCMSFR |
| 6730 | DKTYLTRME |
| 6731 | DLAVMGKCT |
| 6732 | DLMWYEGAS |
| 6733 | DLQTSYHDQ |
| 6734 | DMARALKFF |
| 6735 | DMCTMAYTG |
| 6736 | DMEYSVCHS |
| 6737 | DMKMYEFGP |
| 6738 | DMLMNLMEP |
| 6739 | DMNFTEQYA |
| 6740 | DMTWTMGRQ |
| 6741 | DNACHTPFW |
| 6742 | DNAVLDGVP |
| 6743 | DNMPQPVCH |
| 6744 | DNRFIQDAR |
| 6745 | DNSSEGSWF |
| 6746 | DNVYKGPWA |
| 6747 | DPCPYIDHQ |
| 6748 | DPYPQGRQK |
| 6749 | DQADCNWTW |
| 6750 | DQASYDIHG |
| 6751 | DQCPLRSGT |
| 6752 | DQKSQMQTR |
| 6753 | DQLEFICWP |
| 6754 | DQNSYGCIP |
| 6755 | DQYVPATGY |
| 6756 | DRAVKFSIL |
| 6757 | DRGDQVMQT |
| 6758 | DRGFAHLCQ |
| 6759 | DRSTQDKFD |
| 6760 | DRTVQVRVD |
| 6761 | DRTYTYCWS |
| 6762 | DRVMMQRND |
| 6763 | DRWGMEEHQ |
| 6764 | DRWQIPSKL |
| 6765 | DSATFKSAD |
| 6766 | DSECKNCMC |
| 6767 | DSGGNMQVY |
| 6768 | DSGKQPADQ |
| 6769 | DSHCEVRFE |
| 6770 | DSIQRLSAG |
| 6771 | DSNTGQKAY |
| 6772 | DSVEWTVHE |
| 6773 | DSVRMLGKP |
| 6774 | DSWTGWNFA |
| 6775 | DTCKYLWTP |
| 6776 | DTCSRKHWM |
| 6777 | DTDEYQKAQ |
| 6778 | DTEWKYKHN |
| 6779 | DTKMQHTCT |
| 6780 | DTKWYPSTG |
| 6781 | DTMLGPQMQ |
| 6782 | DTMTCNRHQ |
| 6783 | DTNHATMAC |
| 6784 | DTNITDQWR |
| 6785 | DTQYTRDFV |
| 6786 | DTSWDRIFV |
| 6787 | DTYMEADSI |
| 6788 | DVAEFTGVV |
| 6789 | DVETTKAIN |
| 6790 | DVFKHLQCT |
| 6791 | DVGACFIQD |
| 6792 | DVGPMNGPR |
| 6793 | DVLPAVECP |
| 6794 | DVLTSQELY |
| 6795 | DVMHDSRCS |
| 6796 | DVMPIECQT |
| 6797 | DVNIVCMDD |
| 6798 | DVRSMYVMG |
| 6799 | DVTHGDPAK |
| 6800 | DVTKHALAP |
| 6801 | DVVDGEASQ |
| 6802 | DVVHGGTQL |
| 6803 | DVVKNQGIG |
| 6804 | DVVQNDCEF |
| 6805 | DVYWDECVF |
| 6806 | DWACQEDAG |
| 6807 | DWCWYTCSW |
| 6808 | DWNYRHWFN |
| 6809 | DWVETAQES |
| 6810 | DWYMHDQPM |
| 6811 | DYEQSQKWW |
| 6812 | DYGTAGYPR |
| 6813 | DYIMHHHCG |
| 6814 | DYLCINGFS |
| 6815 | DYNSKMDFH |
| 6816 | DYRPENTGC |
| 6817 | DYTTAIYTD |
| 6818 | DYVRRAWMP |
| 6819 | EAAWVVRKR |
| 6820 | EADNTQRSS |
| 6821 | EADSKFVDQ |
| 6822 | EAFAYDHKD |
| 6823 | EAGFDAPGR |
| 6824 | EAGIDKCCH |
| 6825 | EAHMIGRHT |
| 6826 | EAICVEREY |
| 6827 | EAKLQQEIE |
| 6828 | EAMGCILRG |
| 6829 | EAMHGDAIW |
| 6830 | EARAHQNEI |
| 6831 | EASANWLKC |
| 6832 | EASGLHKIF |
| 6833 | EASNRMIDN |
| 6834 | EATLGGWCD |
| 6835 | EATPFVRFR |
| 6836 | EATVWTWNP |
| 6837 | EAWHFYEWP |
| 6838 | EAYVNKDQW |
| 6839 | ECDFPMGTE |
| 6840 | ECEVSHFID |
| 6841 | ECHHLWCDY |
| 6842 | ECHQMNEDQ |
| 6843 | ECMQRYIGV |
| 6844 | ECNQNWDEF |
| 6845 | ECQNQFGWM |
| 6846 | ECRCIPTVW |
| 6847 | ECRSEMGLP |
| 6848 | ECTRGELTH |
| 6849 | ECVHKHSGS |
| 6850 | ECYHMELMT |
| 6851 | EDTRFHQWT |
| 6852 | EEAEQEEWN |
| 6853 | EEMHDDGDA |
| 6854 | EESQRDMIT |
| 6855 | EFAAEMTPA |
| 6856 | EFCKANGPL |
| 6857 | EFDGCQYFA |
| 6858 | EFEQFTRYA |
| 6859 | EFHQITGSS |
| 6860 | EFMMPMIYD |
| 6861 | EFMPTVDTK |
| 6862 | EFQWWNMSK |
| 6863 | EFTHWMQMQ |
| 6864 | EFTQLAYTY |
| 6865 | EGCWHGHTF |
| 6866 | EGHAFNTFN |
| 6867 | EGKPRLHDA |
| 6868 | EGKTDVMSY |
| 6869 | EGWTVMVDQ |
| 6870 | EHCNHDMNT |
| 6871 | EHCQEMRWH |
| 6872 | EHEAMLVND |
| 6873 | EHEFLGAKS |
| 6874 | EHFRACVVF |
| 6875 | EHFSTYMRE |
| 6876 | EHGYSHTLA |
| 6877 | EHQEQQLVH |
| 6878 | EHSYIMANP |
| 6879 | EHTNLEKYA |
| 6880 | EHTTWHQYT |
| 6881 | EIATHWQKG |
| 6882 | EICHYNEQY |
| 6883 | EIDAMIPMT |
| 6884 | EIDLVTRWE |
| 6885 | EIEEQGWSG |
| 6886 | EIFLNPQYY |
| 6887 | EIGYRHRDC |
| 6888 | EIGYWCGCA |
| 6889 | EIHQVTHYE |
| 6890 | EKHLLNCDS |
| 6891 | EKKFGMSGD |
| 6892 | EKMVAEFQC |
| 6893 | EKQFGVKNR |
| 6894 | EKTWHQRNM |
| 6895 | ELATLVDWN |
| 6896 | ELAWFMNTE |
| 6897 | ELCKTVFLA |
| 6898 | ELGAAVMVC |
| 6899 | ELGFWHGKG |
| 6900 | ELLNPDNPI |
| 6901 | ELMLGNMFW |
| 6902 | ELMMNLIGM |
| 6903 | ELRCRDSDQ |
| 6904 | ELRPITWVD |
| 6905 | ELTKKVKAS |
| 6906 | EMIHRHQVD |
| 6907 | EMKKLCDAC |
| 6908 | EMKPWCQAL |
| 6909 | EMMLVHQPS |
| 6910 | EMNAVFSTD |
| 6911 | EMTKNDSFM |
| 6912 | EMYFMSEGT |
| 6913 | ENIQSIADV |
| 6914 | ENKEYPDSN |
| 6915 | ENMCNQGIG |
| 6916 | ENRLCVGTV |
| 6917 | ENVVAIQTL |
| 6918 | ENYKEIPYG |
| 6919 | EPTASKHDR |
| 6920 | EPTNIPDGF |
| 6921 | EPVRRTVMG |
| 6922 | EPWMPSIAL |
| 6923 | EPWSIGKAA |
| 6924 | EPYCEGVAW |
| 6925 | EQETHEALG |
| 6926 | EQHVIAMDH |
| 6927 | EQIKQLNWA |
| 6928 | EQMKYYWFE |
| 6929 | EQNEQKDMW |
| 6930 | EQVPFCFTE |
| 6931 | ERACSMADR |
| 6932 | ERCHYPYIK |
| 6933 | ERETLGDQR |
| 6934 | ERGMQVWPP |
| 6935 | ERKYIMQFD |
| 6936 | ERRSHTETA |
| 6937 | ERYRGVIAC |
| 6938 | ESCILEEHM |
| 6939 | ESCIMQCMI |
| 6940 | ESCLFHMRQ |
| 6941 | ESENNKFGH |
| 6942 | ESEPNKHNP |
| 6943 | ESFPNKIQG |
| 6944 | ESGVEDMQN |
| 6945 | ESHHKGDHM |
| 6946 | ESKSHWTIG |
| 6947 | ESKVGYELG |
| 6948 | ESLTHEDGY |
| 6949 | ESRAVLSMP |
| 6950 | ESRLTMTCP |
| 6951 | ESVNRYFKQ |
| 6952 | ETAYTTRTE |
| 6953 | ETDHRKHYC |
| 6954 | ETECWHKSH |
| 6955 | ETEMICEFQ |
| 6956 | ETFACPSCR |
| 6957 | ETGMFKFSG |
| 6958 | ETGWKQNIE |
| 6959 | ETHFCIRNS |
| 6960 | ETHQMGMDR |
| 6961 | ETILDQHGG |
| 6962 | ETISRVGMQ |
| 6963 | ETLFNSYGR |
| 6964 | ETLPLYWQC |
| 6965 | ETNKQLNLW |
| 6966 | ETQQNIWSW |
| 6967 | ETQSPMCCL |
| 6968 | ETSCTCEVC |
| 6969 | ETSGQGVLW |
| 6970 | ETTWNVICP |
| 6971 | EVADPMTWV |
| 6972 | EVCPGSIRS |
| 6973 | EVERKVNQE |
| 6974 | EVGNPTQYD |
| 6975 | EVHYCGNCG |
| 6976 | EVIMSHLIT |
| 6977 | EVKYFWHCA |
| 6978 | EVLMDWSWE |
| 6979 | EVLQLLKAD |
| 6980 | EVLYGGQQW |
| 6981 | EVNSGRDWW |
| 6982 | EVQGFIDDR |
| 6983 | EVQHIHFPY |
| 6984 | EVQKILFLQ |
| 6985 | EVQRNGIGM |
| 6986 | EVSHFKVDE |
| 6987 | EVSIQPQMN |
| 6988 | EVSYTIGDI |
| 6989 | EVVHLMMRG |
| 6990 | EVWSPMHNG |
| 6991 | EWGWAWLVG |
| 6992 | EWKMANVMQ |
| 6993 | EWRHAWNCW |
| 6994 | EWRMFINTA |
| 6995 | EWTPFMNEG |
| 6996 | EWYLRHHMI |
| 6997 | EWYQIRHID |
| 6998 | EYAIKICNH |
| 6999 | EYASTACYD |
| 7000 | EYDHIDVWL |
| 7001 | EYEGYKSAA |
| 7002 | EYEPIASGP |
| 7003 | EYEWLMAAA |
| 7004 | EYFDNRCAQ |
| 7005 | EYFHVVGVD |
| 7006 | EYFPTARTF |
| 7007 | EYKSRGREV |
| 7008 | EYMERWFND |
| 7009 | EYSDSMVWP |
| 7010 | EYVRWNKQC |
| 7011 | EYYLLGCAD |
| 7012 | EYYRKQCAI |
| 7013 | FAASSEIYG |
| 7014 | FAAYDMVSE |
| 7015 | FACPEALGT |
| 7016 | FADPHTWFS |
| 7017 | FAEDIHYGA |
| 7018 | FAIEPGLCT |
| 7019 | FATIVVHEE |
| 7020 | FATVSPGWI |
| 7021 | FAYENRLCD |
| 7022 | FCFIEDLAM |
| 7023 | FCFMYPFHW |
| 7024 | FCFYNRCWQ |
| 7025 | FCHLEQCMI |
| 7026 | FCSCCHCRT |
| 7027 | FCVINELGT |
| 7028 | FCYQKLNYN |
| 7029 | FDMQLTAYA |
| 7030 | FDQPIQQCP |
| 7031 | FEHVIRTMW |
| 7032 | FEKWRHPRH |
| 7033 | FGAKYHDGQ |
| 7034 | FGEPANVNA |
| 7035 | FGGLCWLVV |
| 7036 | FGGLVIDAS |
| 7037 | FGKQRNEDY |
| 7038 | FGLWQQAGY |
| 7039 | FHAEWKGPP |
| 7040 | FHTRVNACD |
| 7041 | FICQQGDSL |
| 7042 | FIRGSPMNM |
| 7043 | FIRMIIVDD |
| 7044 | FKAEYQSGH |
| 7045 | FLINPAWQG |
| 7046 | FLWGIRVLA |
| 7047 | FMGKTDVNM |
| 7048 | FMLTMDFWA |
| 7049 | FMNVYFEHA |
| 7050 | FMQVGGINT |
| 7051 | FMTSLRDRF |
| 7052 | FNCEGQLYE |
| 7053 | FNDEYPCSQ |
| 7054 | FNDSEPEVI |
| 7055 | FNFQKTMDC |
| 7056 | FNLEEWWSP |
| 7057 | FNLQYAGLQ |
| 7058 | FNVPFAKDD |
| 7059 | FPRESSNLL |
| 7060 | FQAGLANCA |
| 7061 | FQFTAMWYM |
| 7062 | FQGHSRDGM |
| 7063 | FQLYQNHGA |
| 7064 | FQMTKQTQS |
| 7065 | FQMVEQQPQ |
| 7066 | FQNLLDGCW |
| 7067 | FQTRPSCSF |
| 7068 | FRVRWWHYR |
| 7069 | FRWNIDVTY |
| 7070 | FSANMNADI |
| 7071 | FSDGLNHSD |
| 7072 | FSQHFNRYP |
| 7073 | FSQLMNTPP |
| 7074 | FTFDQPSGY |
| 7075 | FTHPSTDQQ |
| 7076 | FTILLFVHN |
| 7077 | FTLLNSEGF |
| 7078 | FTMRYQKLH |
| 7079 | FVETTQVMN |
| 7080 | FVKSFNNDV |
| 7081 | FVQSSCQQS |
| 7082 | FVVDKYIYA |
| 7083 | FWNNNLFQS |
| 7084 | FWRGITCNF |
| 7085 | FYRLDRCEM |
| 7086 | GACDVLSGE |
| 7087 | GADEPQATL |
| 7088 | GADYFTECH |
| 7089 | GAGHYITAW |
| 7090 | GANAIWTEE |
| 7091 | GAYEYASKM |
| 7092 | GCASEHGRS |
| 7093 | GCCQGSPFA |
| 7094 | GCELIPIRP |
| 7095 | GCEMKYQSF |
| 7096 | GCLELEHYE |
| 7097 | GCTIAEGAC |
| 7098 | GCTQGEQAT |
| 7099 | GCVGAGCDM |
| 7100 | GDCFKQTFH |
| 7101 | GDGNRPTDI |
| 7102 | GDHCSLSEE |
| 7103 | GDHKTIVMF |
| 7104 | GDKYCGVHT |
| 7105 | GDLFWKKIH |
| 7106 | GDYTKMVNQ |
| 7107 | GECTYVGGD |
| 7108 | GFAQYGMAL |
| 7109 | GFEQYDCEM |
| 7110 | GFFCFVTQW |
| 7111 | GFMEHPNSA |
| 7112 | GFMEKCYDG |
| 7113 | GFNALQQAP |
| 7114 | GFNCEKPDA |
| 7115 | GGGHCWQNA |
| 7116 | GGHMKMQME |
| 7117 | GGRAFAGYK |

TABLE 74
Sequences of the 581 to 589 Region in
AAV5 VP1 Capsid Polypeptide that Drive CNS
Tissue Tropism

| | 581-589 |
|---|---|
| SEQ ID NO | Sequence |
| 9118 | AAMESCAEI |
| 9119 | EKYQIHWDR |
| 9120 | VTGTYNLQE |
| 9121 | ATGTYSLQE |
| 9122 | ATGTYNLRE |
| 9123 | ATDTYNLQE |
| 9124 | MWTKFNEYG |
| 9125 | QTLGGHMWT |
| 9126 | TLCHTDNMV |
| 9127 | GIAYEVLWD |
| 9128 | SRLPVHECP |
| 9129 | TTGTYNLQE |
| 9130 | MWTEFNEDG |
| 9131 | TFVYGERDR |
| 9132 | GQQTIEMSY |
| 9133 | NYAMRRDSY |
| 9134 | KPLASIGQY |
| 9135 | AAFVMNGDG |
| 9136 | RVAGEGIQP |
| 9137 | TCGHTRFFI |
| 9138 | ESAEPRMIP |
| 9139 | TTSQNSEAM |
| 9140 | NTQVISRWT |
| 9141 | AGCNTQNGH |
| 9142 | VGPLYAYYS |
| 9143 | QHSRAYMWY |
| 9144 | DIRIVSDGK |
| 9145 | TMAQKGLWF |
| 9146 | QCEYSNCCY |
| 9147 | NGTQCLNMD |
| 9148 | MTDNNGPLF |
| 9149 | CPEHNQVGV |
| 9150 | EMYVQRHSL |
| 9151 | LINSIVCGD |
| 9152 | ACKPCNGWD |
| 9153 | PLTIEVNCT |
| 9154 | SKGWADLCP |
| 9155 | DMTLQSVSS |
| 9156 | RVTIMFTGT |
| 9157 | RMFWDTSDR |
| 9158 | SMQYFFKTE |
| 9159 | SFKGFHLQN |
| 9160 | TTYPHNTHG |
| 9161 | KSTMLEHHD |
| 9162 | TCKWYEESI |
| 9163 | DICQKQSPN |
| 9164 | ILEAIVNFW |
| 9165 | DYEVCGNWS |
| 9166 | EWNQVWWHD |
| 9167 | SPNGRGLCG |
| 9168 | TSMEFTQHQ |
| 9169 | ASFFLNPLE |
| 9170 | MITNNCNIS |
| 9171 | MAEQMERDF |
| 9172 | VLTTMWKNN |
| 9173 | IATHNHILN |
| 9174 | MYDTMGAWC |
| 9175 | CKQLFCELG |
| 9176 | MWTEFNENG |
| 9177 | ECVSDYACC |
| 9178 | AGFSQFDCF |
| 9179 | RGKPILNQQ |
| 9180 | KSAQVYWHP |
| 9181 | AIGPALCLT |
| 9182 | QNKHNWWIM |
| 9183 | MASQGGQVR |
| 9184 | CGHLLRRAE |
| 9185 | WGDNKNFMG |
| 9186 | FVFAMIPGF |
| 9187 | TFMFNSEWS |
| 9188 | MAQPYGRTA |
| 9189 | CKNHYMMAA |
| 9190 | NSGVLIGME |
| 9191 | LAESKKRNK |
| 9192 | AFNGANSWT |
| 9193 | YTELMKKAC |
| 9194 | ETNDNMILH |
| 9195 | QFMMGQQTP |
| 9196 | VKAWWHDHQ |
| 9197 | GKIAHDQLN |
| 9198 | QVHMQRYYG |
| 9199 | MFVVNQNWA |
| 9200 | ILINDREWG |
| 9201 | DVTQTRYCY |
| 9202 | QTGVMAAFG |
| 9203 | ESRQVQDCY |
| 9204 | IMNPTAQNN |
| 9205 | CCCVPRSIY |
| 9206 | NRIQMTDFQ |
| 9207 | QPYWVAVDP |
| 9208 | KMLHKVWID |
| 9209 | QWMHCQLSR |
| 9210 | GHMFETFWG |
| 9211 | NCPMYDMIS |
| 9212 | CCCQFSFWN |
| 9213 | TLEPAYRHE |
| 9214 | NLSNLVSCR |
| 9215 | DRKQADQIY |
| 9216 | QPKMWMMAS |
| 9217 | KEFQLQMEP |
| 9218 | QMKGATEFC |
| 9219 | LETMMRHGE |
| 9220 | LCKFANGAA |
| 9221 | FAQQIINAS |
| 9222 | IMYLKRCCT |
| 9223 | EKRLEGWVY |
| 9224 | NVWSPAVRD |
| 9225 | DEYQQESFQ |
| 9226 | EICMRSGFI |
| 9227 | AVKAPSQMS |
| 9228 | QTDILRDVQ |
| 9229 | DNYWIVPFA |
| 9230 | INMPVSKDQ |
| 9231 | ENQSTMSHT |
| 9232 | FNTPYAHIG |
| 9233 | LESVKMDCN |
| 9234 | WCEAVGDYA |
| 9235 | SMSHNGQCL |
| 9236 | HAETDHVGL |
| 9237 | QCTLEISYV |
| 9238 | SGQPVDCMF |
| 9239 | AAAWSCMQW |
| 9240 | RTVPTQEMS |
| 9241 | LTPRSEGLP |
| 9242 | GDVSYDAEA |
| 9243 | QHKWHDVGV |
| 9244 | TAFQSLQQM |
| 9245 | REFAISGGN |
| 9246 | PHAETHWYT |
| 9247 | NLAPMIQVC |
| 9248 | STGSSKDQD |
| 9249 | HFEVRSVQR |
| 9250 | TMRLFWDMG |
| 9251 | NFVNHQPLP |
| 9252 | GKGQWTAQE |
| 9253 | VHGQIDLSL |
| 9254 | YISEYVMMV |
| 9255 | GVRNALNLS |
| 9256 | HMEQCWCNE |
| 9257 | MFFPGFVAH |
| 9258 | QCFLWNDCQ |
| 9259 | LIHWLKHDL |
| 9260 | KCAWIEVTP |
| 9261 | YPVHMMPPL |
| 9262 | PCCWEQSTN |
| 9263 | CSMPQQCME |
| 9264 | RRTDHGEPI |
| 9265 | GSKIIMHTE |
| 9266 | SQMKYAHNQ |
| 9267 | EFNWLLWSG |
| 9268 | AEDTPYTIY |
| 9269 | RTAEDCPFQ |
| 9270 | AKCTILNFQ |
| 9271 | WFSIIMSKL |
| 9272 | QTAEWYCGA |
| 9273 | MHGIARMQQ |
| 9274 | VTKYCDHEQ |
| 9275 | DFNGSNWLP |
| 9276 | IHFTSQSCL |
| 9277 | QVKWTADPD |
| 9278 | CIQPTWDSY |
| 9279 | DVKAFMGGV |
| 9280 | DPGWYGLAP |
| 9281 | DHLSNDAEM |
| 9282 | NSIGWDSAS |
| 9283 | TNFPTSMHG |
| 9284 | RQTTYDCLD |
| 9285 | SAKCCINAV |
| 9286 | VHHKTNRSY |
| 9287 | FGSMQANDR |
| 9288 | AVETGESAM |
| 9289 | MVENGLRLQ |
| 9290 | AIEHGAWKC |
| 9291 | SMMEQKEQQ |
| 9292 | AFLHGQMWQ |
| 9293 | IEFTHSRKV |
| 9294 | NWKLPTCMT |
| 9295 | EHKGCMQAI |
| 9296 | ATGTYNLQE |
| 9297 | NGNHYNAMR |
| 9298 | QHEQSNHIW |
| 9299 | IGRLNASTP |
| 9300 | MSNLEYYEA |
| 9301 | TSCCYESSS |
| 9302 | WINEANCLM |
| 9303 | VESAMINQA |
| 9304 | EPRLYCAQE |
| 9305 | WPNIKHQPI |
| 9306 | FTHSCCCLS |
| 9307 | SFSDTHLGW |
| 9308 | SDSKVQWGM |
| 9309 | WDGWVTTHF |
| 9310 | QTNGHPYFP |
| 9311 | QTSHQDQDC |
| 9312 | HMGIAYNDH |
| 9313 | TFVRKPSLM |
| 9314 | QQLCYMSDE |
| 9315 | KVAEGQNML |
| 9316 | LHYRPTEQC |
| 9317 | KTGMGDLFL |
| 9318 | AQCHDMWGH |
| 9319 | ATGLTDHQW |
| 9320 | QWAPLQTWA |
| 9321 | SPHNCQHMG |
| 9322 | ISMRSECED |
| 9323 | KIAAWNDFL |
| 9324 | RFDQFWDTY |
| 9325 | HQCNCLLSW |
| 9326 | MAMWIADTH |
| 9327 | AVHTADVCC |
| 9328 | SCAPDTQFG |
| 9329 | CWILHHRCS |
| 9330 | QVGIEFVVC |
| 9331 | TIVVIPIKS |
| 9332 | MPWLDKPPC |
| 9333 | YMSWYQIRN |
| 9334 | AVYLDHVFS |
| 9335 | KACMSHQGR |
| 9336 | TSIEGLKTM |
| 9337 | AWSYTGACY |
| 9338 | QCVDCPPYC |
| 9339 | VTCNWCVMP |
| 9340 | KCADLIIRS |
| 9341 | NVCDNGHEI |
| 9342 | MMKRNRFIC |
| 9343 | SRVMIHVYV |
| 9344 | WKATPGGQC |
| 9345 | WTTSIAVDT |
| 9346 | DTHHVHGWQ |
| 9347 | MGYAKHDSH |
| 9348 | GCEEVGRCQ |
| 9349 | KTMGIGGGP |
| 9350 | RVWSLEWHL |
| 9351 | EQPNMGDYG |
| 9352 | FGEFTMDEN |
| 9353 | KFGFMYNEF |
| 9354 | ERFHSYPLG |
| 9355 | FLYTFMFQH |
| 9356 | GSVMLMVIR |
| 9357 | KMAGIGCWY |
| 9358 | RTRISQMFG |
| 9359 | AQARTIGLG |
| 9360 | MIRPSINGS |
| 9361 | TKGRNDHME |
| 9362 | ATIAQWCVH |
| 9363 | RYNNCPNLW |
| 9364 | FKIEWSQDI |
| 9365 | KKHKGWCRA |
| 9366 | GAANRIQSW |
| 9367 | NWTGNMQWA |
| 9368 | CMEVTCFVV |
| 9369 | RVEMGCEPR |
| 9370 | SQIQIRQAD |
| 9371 | KIGCMYSSQ |
| 9372 | MWHELIADQ |
| 9373 | PQEAQIGAP |
| 9374 | KTQIHMGDE |
| 9375 | LPSHCNLGS |
| 9376 | KVESTTQLC |
| 9377 | AYAMRNYTS |
| 9378 | IIRQSEQYH |
| 9379 | AHAWLQHWA |
| 9380 | HEVMMCPAP |
| 9381 | EVAFTTSSD |
| 9382 | PLNQEQIPQ |
| 9383 | TSVIRLFWI |
| 9384 | QFAQLWCAK |
| 9385 | GIWVRDTYN |
| 9386 | LCTSTERQG |
| 9387 | DRVNLMWKD |
| 9388 | CYMNFIVDG |
| 9389 | QISKPSVQC |
| 9390 | GITMHPMHH |
| 9391 | LPPPEEKWL |
| 9392 | TTTNQFMFQ |
| 9393 | RCGEIMGLD |
| 9394 | MQLRIHTNS |
| 9395 | ICHIRTRGN |
| 9396 | MSGPNDTEF |
| 9397 | NTQYSAKND |
| 9398 | KQKTQNRNA |
| 9399 | TRETMNEWH |
| 9400 | ERQAHNHYA |
| 9401 | SKGWADPCP |
| 9402 | RTEAHLPAP |
| 9403 | GNECFHNVE |
| 9404 | ECIFAPRMS |
| 9405 | YFPSIQCYE |
| 9406 | SSIYKEIQE |
| 9407 | THVKRFDYE |
| 9408 | LYMMALLPT |
| 9409 | SGALKNREE |
| 9410 | EASIQASWN |
| 9411 | ACIPQMHSA |
| 9412 | LHSYQWRLG |
| 9413 | ACVERLHHK |
| 9414 | TVNWYSGFG |
| 9415 | IAKQNFMKT |
| 9416 | FTQAIHHPD |
| 9417 | YHCCFAMQS |
| 9418 | NLERCNTMM |
| 9419 | HMHNLPVKP |
| 9420 | HCWVNEKCE |
| 9421 | FADKWTNQF |
| 9422 | RQAWPKDGM |
| 9423 | CSESYRGPD |
| 9424 | VLKSNLTGM |
| 9425 | NAYYDSHCE |
| 9426 | YPAQYTAFS |
| 9427 | GLFEGSPGA |
| 9428 | VTMIHNAFH |
| 9429 | IYMSTGNTP |
| 9430 | YIGMKSCSG |
| 9431 | MFMTNVNSF |
| 9432 | IAQSEADYW |
| 9433 | AVHNVKDVH |
| 9434 | HIHMSDRGA |
| 9435 | GLFHPQFGQ |
| 9436 | GPRHIEEDG |
| 9437 | AYHCYMSWM |
| 9438 | ITASIKDQW |
| 9439 | PAHINDQYM |
| 9440 | AAMSSSHSY |
| 9441 | MNSFYRAEW |
| 9442 | QATGMHRWQ |
| 9443 | SSRMQMQGP |
| 9444 | MEHTMDFGV |
| 9445 | YLEMYSKDW |
| 9446 | QWFTKGVGE |
| 9447 | KNCIMLASN |
| 9448 | AYAPSKNAM |
| 9449 | GLSMVLHFV |
| 9450 | SPQQPSVFH |
| 9451 | PGGCYRMME |
| 9452 | KTCLCIFTK |
| 9453 | ACHTGQSGM |
| 9454 | DIEFLDCEN |
| 9455 | WTLQVMKIN |
| 9456 | DVKSWAVCD |
| 9457 | LCAVCGDCD |
| 9458 | DQEAQVVTG |
| 9459 | HFQPVFMQP |
| 9460 | GHLFNNNEW |
| 9461 | YDYWMQAPT |
| 9462 | ASMHEETCL |
| 9463 | DCVLTMVDK |
| 9464 | MLPLEMKNL |
| 9465 | IMTIIPYGP |
| 9466 | LIEKQRWML |
| 9467 | VSVQSRAQL |
| 9468 | THSSCWYQD |
| 9469 | VFNLMLQDK |
| 9470 | HIKVEHEEV |
| 9471 | MMLGYMEQD |
| 9472 | TQCKQLTVM |
| 9473 | QSSHCKQMV |
| 9474 | WGGMPNNQG |
| 9475 | FLTCDQFEW |
| 9476 | RVNGSTCRH |
| 9477 | ETHAAMNSM |
| 9478 | DAKLTMHSA |
| 9479 | RYPCCQSPF |
| 9480 | QGENAQHHN |
| 9481 | TSNHNMALE |
| 9482 | NPGWQTIGQ |
| 9483 | CAWHMKQGN |
| 9484 | NVMGTQAKE |
| 9485 | TYIQYPTNA |
| 9486 | DGMHRELYS |
| 9487 | QSAHPEPMC |
| 9488 | QAKMILDGT |
| 9489 | VHVHCMIGS |
| 9490 | TAALGHSFC |
| 9491 | DTRRNLCCD |
| 9492 | ENTMWQSSQ |
| 9493 | NQREWHGLA |
| 9494 | PDLWYEKSS |
| 9495 | ESSQEIKTC |
| 9496 | YVDHWTLGD |
| 9497 | NHSLCWDSK |
| 9498 | SECEVLCSL |
| 9499 | DQRHGNVSP |
| 9500 | MCFPYSCAA |
| 9501 | ASKQGTHDY |
| 9502 | GNKHCMLGT |
| 9503 | GTKLIWPYQ |
| 9504 | QCTAHNTCM |
| 9505 | ACDQVNTVQ |
| 9506 | IECSPRESI |
| 9507 | RCADNPYFR |
| 9508 | QESMCNYWQ |
| 9509 | AGMYKCQED |
| 9510 | SQAHQNRTC |
| 9511 | MGRQKDNVY |
| 9512 | NANYWFYYD |
| 9513 | EFVLGCQGI |
| 9514 | QRSVFHQRS |
| 9515 | PIWQGYWPW |
| 9516 | HSMMPGWPS |
| 9517 | GVHHRCRMT |
| 9518 | IQAVMASGW |
| 9519 | IAKIGNCVW |
| 9520 | CVDTNRMWQ |
| 9521 | ARWFSTVEQ |
| 9522 | VFKPLDKRY |
| 9523 | EVRIQCKID |
| 9524 | NGVGRSGDN |
| 9525 | CVEQYVEQG |
| 9526 | LSAQRLVCD |
| 9527 | KGNPLDGDT |
| 9528 | PVICVWHDN |
| 9529 | EIHMFGQSE |
| 9530 | YCECGHWPN |
| 9531 | TYESLQNSW |
| 9532 | INYISFVDR |
| 9533 | MWGCKLFVC |
| 9534 | CQMLCRPPV |
| 9535 | RDQCRMESG |
| 9536 | WVHPYSYCA |
| 9537 | QSRCVDNTV |
| 9538 | GRALGKKQG |
| 9539 | SNVACIQAF |
| 9540 | HQYVHNWRQ |
| 9541 | SVLHVQALA |
| 9542 | ATMNLSWGL |
| 9543 | WERCLELNQ |
| 9544 | TKMIIFDSW |
| 9545 | CWECCTDSG |
| 9546 | YSDPKNMSI |
| 9547 | FPKGSECNW |
| 9548 | EAGCKMWPT |
| 9549 | KDGRNPHCL |
| 9550 | LEIGGKTRT |
| 9551 | YKGMEKNPV |
| 9552 | DLIVATEWF |
| 9553 | WHTIMTYES |
| 9554 | DTAFMHHHV |
| 9555 | FQWRTEMNN |
| 9556 | SGDWDFGEW |
| 9557 | MNLQIGSKG |
| 9558 | CNGYWMCMS |
| 9559 | SQERMDYDG |
| 9560 | SVGYIYDQS |
| 9561 | GTWSDPWPN |
| 9562 | YPTMTFPEE |
| 9563 | AAQYMGIMK |
| 9564 | HTVMEWSPT |
| 9565 | GQHTTSHAG |
| 9566 | RYQSHCWLH |
| 9567 | LSTMFPEQT |
| 9568 | NIGVIPHIQ |
| 9569 | PWTTPENYT |
| 9570 | YNCPWDQQY |
| 9571 | QWHDDQWST |
| 9572 | DYAQPTGPD |
| 9573 | FKHSLREWT |
| 9574 | AARMVGEFY |
| 9575 | GSMPSTVPH |
| 9576 | ALAKFQCWS |
| 9577 | AADIFAIND |
| 9578 | QHHWKWSTE |
| 9579 | AAGAFLSGC |
| 9580 | FDVQFMRVD |
| 9581 | ALQYCQGKD |
| 9582 | NERDQCQWP |
| 9583 | CRMITMMNE |
| 9584 | EWHARHHYW |
| 9585 | STSWLNDLT |
| 9586 | DEGYIMFAP |
| 9587 | QMVTQLNWE |
| 9588 | VVTQYVDAP |
| 9589 | TVIRDSRSY |
| 9590 | DEAKKSCWQ |
| 9591 | AVNEAQDNR |
| 9592 | NERSGSVLE |
| 9593 | TYASTRNFH |
| 9594 | IRRSMQQQN |
| 9595 | GFQRTHVDQ |
| 9596 | HKHRMCKAR |
| 9597 | TAGQQPTFM |
| 9598 | VQMNLDAAP |
| 9599 | KDHQLWEVW |
| 9600 | GDCIIVISW |
| 9601 | RDVGEDNSA |
| 9602 | PWMHTMLNP |
| 9603 | ETSTHCCKV |
| 9604 | QVNMFMMPC |
| 9605 | NADMSFRNR |
| 9606 | FRLDYDYKN |
| 9607 | TNAAYEMWF |
| 9608 | TTTITNRLC |
| 9609 | KIQPMMVKA |
| 9610 | LFRAYAWFQ |
| 9611 | DVIRDMIAE |
| 9612 | ALTHDDGRT |
| 9613 | KTYSAMYSP |
| 9614 | NCMWISSTQ |
| 9615 | TILPKKTLC |
| 9616 | VTTHMNCAD |
| 9617 | PPRFLQTTG |
| 9618 | VSVPKRQGP |
| 9619 | VFGSTRSAA |
| 9620 | TSDQCGNED |
| 9621 | GAKYIQNTD |
| 9622 | MRYHWDEAG |
| 9623 | SSEYSQFAI |
| 9624 | GVFWPDSFR |
| 9625 | GAYSLHDWR |
| 9626 | MTALPPSKN |
| 9627 | HGFKCQYSF |
| 9628 | YGGSEHWGP |
| 9629 | TTAYHRSDH |
| 9630 | NTHTGNDRL |
| 9631 | YPQPCSCHS |
| 9632 | VSGFDRVAF |
| 9633 | SRLYKKRLF |
| 9634 | CCKSKEVCA |
| 9635 | TYDTRNHCL |
| 9636 | SFKSVSQYN |
| 9637 | QAAVTKEYW |
| 9638 | EIIQAHLIN |
| 9639 | KTGATNLAW |
| 9640 | GQFRSGAYR |
| 9641 | PMMIIPYVD |
| 9642 | EPLVCISAG |
| 9643 | KGGLSTTNA |
| 9644 | KSTYYNLKW |
| 9645 | DVGIGVDQG |
| 9646 | MCDNFPSAH |
| 9647 | IKHITRNTY |
| 9648 | GNMHNFSAW |
| 9649 | CSIQINNCR |
| 9650 | MREKLVHDC |
| 9651 | EQPHISIKG |
| 9652 | EMSISHGCH |
| 9653 | RSDEMYVTP |
| 9654 | SFSYDVVMN |
| 9655 | GDMCEDMCT |
| 9656 | TVTQGSIGH |
| 9657 | RKDQSDLWP |
| 9658 | MAAITQNVA |
| 9659 | NSTPMACME |
| 9660 | PLSSEWRWA |
| 9661 | CHLKICMHS |
| 9662 | DVWWTLTRD |
| 9663 | DATTSTECH |
| 9664 | AMLQKKRQD |
| 9665 | TIAMPSWTY |
| 9666 | NGYVVDNHS |
| 9667 | GHHQCYKAD |
| 9668 | SANQRYHDW |
| 9669 | DCAWMQKEW |
| 9670 | KVNCRMIIM |
| 9671 | KCRYKDCQC |
| 9672 | NTSELLMAQ |
| 9673 | QGKQLYHMC |
| 9674 | NWYTTWIDE |
| 9675 | QAGNSMWAK |
| 9676 | RMYATEHCS |
| 9677 | NDMQMNWVH |
| 9678 | MHDGYRGMQ |
| 9679 | WVEPEQCFG |
| 9680 | TRCCEPYFQ |
| 9681 | LSAKQQFFC |
| 9682 | ECTKSMQSG |
| 9683 | NCHQLGDNM |
| 9684 | PCQQLMCFM |
| 9685 | YTKYNWKAA |
| 9686 | GTIRMPEHR |
| 9687 | VCHNPDPFT |
| 9688 | SLTQYQWRG |
| 9689 | TLNSRHPEC |
| 9690 | IMTMQMDTA |
| 9691 | YMHFRQYPI |
| 9692 | QTMCITIHP |
| 9693 | QEAGYTRHN |
| 9694 | MNHQCMLWD |
| 9695 | PWYNWDRRC |
| 9696 | DASQIPITF |
| 9697 | IHRVVCRDH |
| 9698 | HHMPIWLGL |
| 9699 | AEVPYVMQQ |
| 9700 | CVLSRSTMR |
| 9701 | AKRECTHIP |
| 9702 | TISQIPWVR |
| 9703 | STRPIACHS |
| 9704 | AWKMAHNSF |
| 9705 | RNDNTCHAY |
| 9706 | IEHCSNRYA |
| 9707 | AVEDYWRYF |
| 9708 | KHIVGQDGG |
| 9709 | ERFKTFCEE |
| 9710 | LVQKWMRTC |
| 9711 | DARDQQMGT |
| 9712 | NMPSLRTRI |
| 9713 | GSQQTTLSC |
| 9714 | GYFYALFFM |
| 9715 | GFRILDACL |
| 9716 | ETDCMEGGH |
| 9717 | YPDTADIFW |
| 9718 | QFMGVSSHS |
| 9719 | VHPGFEKLA |
| 9720 | NTFEQCTEH |
| 9721 | NNRRQFGYV |
| 9722 | QQSFFFGEQ |
| 9723 | QSMVACYTI |
| 9724 | DSMVYNRPQ |
| 9725 | TRDAKVANG |
| 9726 | SVKDPMDWG |
| 9727 | TMTVRMEQP |
| 9728 | ACNTLEHMA |
| 9729 | KIRDCDGNV |
| 9730 | IAYHYDQQN |
| 9731 | QTNEEEDGN |
| 9732 | NHILTPVVF |
| 9733 | QSYPTMYQC |
| 9734 | ECGCMYWGY |
| 9735 | EGVNRPNMK |
| 9736 | HATGLVNFM |
| 9737 | NMFQLKPQN |
| 9738 | NDVWQPHPN |
| 9739 | TQPEDHWLA |
| 9740 | IWITCPYAA |
| 9741 | TCHPNMSVP |
| 9742 | SNVMATKNW |
| 9743 | VDRLFRMEY |
| 9744 | FNIQCLFSR |
| 9745 | PYGPIEYDE |
| 9746 | DYFQYNEAP |
| 9747 | TTCYFVASQ |
| 9748 | AVVCKMNYH |
| 9749 | QVSFVGCNH |
| 9750 | ATGTYNLQA |
| 9751 | RKWMNAKAT |
| 9752 | ENHPTWEKS |
| 9753 | KAVLFLKDC |
| 9754 | ARYMYEKDA |
| 9755 | SHAQPIASH |
| 9756 | ATGTHNLQE |
| 9757 | IPRPFFTMH |
| 9758 | SCQLLNDGG |
| 9759 | DGEFLHEVL |
| 9760 | LFMTYSARN |
| 9761 | QVSFNARYA |
| 9762 | DILAFDGSC |
| 9763 | NTGVKFCAC |
| 9764 | IGQYFNFHF |
| 9765 | AEHMVVDHE |
| 9766 | MQHLPPAMT |
| 9767 | RAEQKFVLL |
| 9768 | QGKEFICGP |
| 9769 | KCNDIVLYH |
| 9770 | FQCKMFRIW |
| 9771 | STWSANRMT |
| 9772 | QCGCAPMDS |
| 9773 | CCLWSYWAF |
| 9774 | QFCICCDGM |
| 9775 | CVFSRNNYQ |
| 9776 | LTCMHASTP |
| 9777 | GYAQRGFFM |
| 9778 | HLMPRNQCF |
| 9779 | DIATMRNTW |
| 9780 | GKVQWSAQE |
| 9781 | AHQTAWAWI |
| 9782 | GCQCTGHPI |
| 9783 | EPLYSWVGI |
| 9784 | EIYSQLTDL |
| 9785 | KMEVNMSEL |
| 9786 | NVTTMFGQK |
| 9787 | CKVDIHDYM |
| 9788 | ENCMSRMKP |
| 9789 | HPLPNTEIC |
| 9790 | KIGVNSHYD |
| 9791 | GGDGRVHNK |
| 9792 | SIRYDFYVP |
| 9793 | SNTPMPYHG |
| 9794 | HVTMNQLYS |
| 9795 | QHLMTFSHE |
| 9796 | GREITQVMS |
| 9797 | GRWMCNEAE |
| 9798 | TNMLAMRGD |
| 9799 | MYTGMRCWR |
| 9800 | LAAIQHCAM |
| 9801 | RQIGTGNME |
| 9802 | AQQQILNSN |
| 9803 | CASSKSHYL |
| 9804 | CLYHSMMKQ |
| 9805 | FKPFAQQWN |
| 9806 | KAVSYHHDG |
| 9807 | ICHTEVMFN |
| 9808 | QCQSQEWFH |
| 9809 | WCTQFDEYT |
| 9810 | PACKEWCFC |
| 9811 | IHNRYAQAP |
| 9812 | ASSRFGSYG |
| 9813 | EFGEMLGVD |
| 9814 | CEMQVQLCV |
| 9815 | DFMWYHICS |
| 9816 | EFDWLLGTI |
| 9817 | IATKSNCHK |
| 9818 | QPFCFSIGA |
| 9819 | QFHVWFEQK |
| 9820 | EMSPIDWHY |
| 9821 | ARAYYDSYS |
| 9822 | TVRHQMDAY |
| 9823 | VKFRITTEC |
| 9824 | SDHQYRGCL |
| 9825 | TSSDHYALP |
| 9826 | GTDLSNHFQ |
| 9827 | RAERYFKDE |
| 9828 | SKGDLCHQQ |
| 9829 | EAQPSEING |
| 9830 | TVCGSFHGC |
| 9831 | QTPKYFVHQ |
| 9832 | KTDKAGLME |
| 9833 | VPTGRRVKL |
| 9834 | GAQSMQFSC |
| 9835 | TQEACHHDP |
| 9836 | MKTIMQDYF |
| 9837 | MFSHGHVMF |
| 9838 | SVSTKPHVE |
| 9839 | RAIYNDMTE |
| 9840 | MFTPCQEGL |
| 9841 | KNAVLNASD |
| 9842 | DVYMAVVGS |
| 9843 | CIMLNGCFQ |
| 9844 | RHWYIALYA |
| 9845 | CWQLFNKQN |
| 9846 | KPCNPVGQE |
| 9847 | SSVVMAKQP |
| 9848 | NAKYSMLYG |
| 9849 | GMWAQDSSG |
| 9850 | YPKRCAEGD |
| 9851 | VKHTAVDVP |
| 9852 | GEGVEQMDG |
| 9853 | MCRPVMMIE |
| 9854 | GTNAYMAHK |
| 9855 | NIQLLHSYC |
| 9856 | MWAETADPA |
| 9857 | QIKTHSNVF |
| 9858 | RLEITRYIW |
| 9859 | VTMTTKSVW |
| 9860 | TMPDRDPTY |
| 9861 | TVWIQSYSN |
| 9862 | CVKPFLTGW |
| 9863 | LIWSVYQDE |
| 9864 | MPHHSPDCW |
| 9865 | HGYQMAYAQ |
| 9866 | QVYMMEDNM |
| 9867 | VTNWLSLQS |
| 9868 | MVFLEQHGR |
| 9869 | KRTDLHAAD |
| 9870 | QTGTGHWFE |
| 9871 | TMGLGMNAI |
| 9872 | VLKSCDRHG |
| 9873 | TYWCPLMMN |
| 9874 | ANGTRECQS |
| 9875 | DSQYHVKCG |
| 9876 | MALPLHVLS |
| 9877 | TIWVHEYGM |
| 9878 | QTQICHSWM |
| 9879 | TEEVQFVVH |
| 9880 | DSATFKSAD |
| 9881 | CGCQVKCHT |
| 9882 | EAYITYNKA |
| 9883 | CTHHNLKAA |
| 9884 | AWSDLMNCW |
| 9885 | MTIKMEQQY |
| 9886 | KATHCDKYV |
| 9887 | AHEDPNTWC |
| 9888 | AWMCCARHD |
| 9889 | QWEEVWWEA |
| 9890 | NAIPYFVQA |
| 9891 | ITSHWLSTN |
| 9892 | KSPCCMSVQ |
| 9893 | AGYQCGYNE |
| 9894 | ETNGVEDWY |
| 9895 | SMKQMTKSF |
| 9896 | YACWLQDAL |
| 9897 | KWEQRSKMS |
| 9898 | KTQTHTSEP |
| 9899 | GNVLLGCQP |
| 9900 | HAKQANGMV |
| 9901 | KCEGEEVGR |
| 9902 | HSTNLFEAK |
| 9903 | GWADFRELQ |
| 9904 | ETEIRQMDG |
| 9905 | KITVHDRIP |
| 9906 | VCGWWHQYW |
| 9907 | IADDYFMCH |
| 9908 | GVWMPHGWR |
| 9909 | MSLQHHFCT |
| 9910 | YNNDMKQRR |
| 9911 | QGVACLDQL |
| 9912 | RITQGESGM |
| 9913 | ASGHMTMCM |
| 9914 | NTNRAHYWH |
| 9915 | TIKQQMEMM |
| 9916 | CQNTAVRAI |
| 9917 | AVAEIRPEP |
| 9918 | YSFATGNAA |
| 9919 | SYLWRSVYC |
| 9920 | AVDHYSNGA |
| 9921 | ASIATMDRI |
| 9922 | NHKMVQDAL |
| 9923 | QRALPHPIK |
| 9924 | LTHNTPFPK |
| 9925 | SIAQDQVWS |
| 9926 | CCRQIWVQY |
| 9927 | CWGPIMHPF |
| 9928 | GNKSPDECF |
| 9929 | GHRPKLCDQ |
| 9930 | QVACHMTNP |
| 9931 | QYVQCAAKD |
| 9932 | QDEPMNLSC |
| 9933 | GMRTHSVMN |
| 9934 | GTNHRDCLV |
| 9935 | LMKYQWIGN |
| 9936 | KIDQTQWQR |
| 9937 | TLHYFHGYQ |
| 9938 | QSQWWDTCN |
| 9939 | WKPEQRHTP |
| 9940 | IYCLCQDSD |
| 9941 | DMKQKAEYT |
| 9942 | CIPTYRHFG |
| 9943 | VRQFGTWAT |
| 9944 | ICIRGARAD |
| 9945 | TSSKHLVFD |
| 9946 | MIKLINCVN |
| 9947 | IESNTNKFP |
| 9948 | GIKKPNEDN |
| 9949 | AMTRCKAGH |
| 9950 | HGNMVLVIG |
| 9951 | LIQSYPHQC |
| 9952 | GGALARSSI |
| 9953 | QKAIVAQND |
| 9954 | KTLPFAIYH |
| 9955 | KIQPTNHHE |
| 9956 | KMGMHDMAM |
| 9957 | QENDERIWY |
| 9958 | APFFTFTNH |
| 9959 | YSDMFHKDH |
| 9960 | RMVLAYGNF |
| 9961 | GQDCWGLAM |
| 9962 | PRWQSEKCQ |
| 9963 | NTAKLGWGL |
| 9964 | MVMMREPSN |
| 9965 | MDGRDEIIH |
| 9966 | VCRLDNGPQ |
| 9967 | MTGIGKCWN |
| 9968 | EIHTGRPGS |
| 9969 | QIIYRCRST |
| 9970 | KCLPYMEMF |
| 9971 | PNGYRPFCC |
| 9972 | QGQSGTQYA |
| 9973 | LVSQWDYDM |
| 9974 | ESCHLQHGE |
| 9975 | TSEPHCSYA |
| 9976 | LSVQAQWDS |
| 9977 | FVPWCHSFG |
| 9978 | MKEDNKFAV |
| 9979 | KTGWDSHWS |
| 9980 | TGMTFNSFR |
| 9981 | NGYLVHACQ |
| 9982 | QVEWFMAQY |
| 9983 | YTWPYPETH |
| 9984 | IQCKYIHFT |
| 9985 | SNVNLMNSR |
| 9986 | ATKFFCSSE |
| 9987 | MREMPMVGA |
| 9988 | SMQEYQKHI |
| 9989 | KSMPPGQGD |
| 9990 | ECRMAVQSC |
| 9991 | VRWGHTCFY |
| 9992 | VKRLENEWH |
| 9993 | TNRCHDDRG |
| 9994 | TTKNAISFE |
| 9995 | EAELENVNM |
| 9996 | IQNPAMWGT |
| 9997 | CDWMANVSV |
| 9998 | EQTHTWLCR |
| 9999 | AKYLPELYY |
| 10000 | EIHEFRASN |
| 10001 | WLIGMWNTG |
| 10002 | WHVQSKRWT |
| 10003 | TTDQQCTGH |
| 10004 | NISPYEMAV |
| 10005 | YYNHKIVEP |
| 10006 | AKPTHDVGS |
| 10007 | HTNQQANAP |
| 10008 | VTKLAENVY |
| 10009 | DVCDDHKPN |
| 10010 | IAHWKCHYP |
| 10011 | YWEKVPCMK |
| 10012 | NWEFNARHD |
| 10013 | VRLQPQHNK |
| 10014 | KCTEYVHTC |
| 10015 | VPTPWMGIS |
| 10016 | HHFHVNLDI |
| 10017 | DSRTQGGLC |
| 10018 | VRADNMPVM |
| 10019 | LLPIMHTHW |
| 10020 | ISLVWMHYP |
| 10021 | MWTEFNEHG |
| 10022 | MWAEFNEYG |
| 10023 | QVHIGSWPP |
| 10024 | YEMTSKCTY |
| 10025 | HMSQAGVVS |
| 10026 | DQSMMTIGQ |
| 10027 | MPSWYIAQA |
| 10028 | GAGPVQSEE |
| 10029 | FGGMTPYGV |
| 10030 | VCSNVRSYC |
| 10031 | HGNSFLVHW |
| 10032 | MWTEFNEYW |
| 10033 | DITSLGCIM |
| 10034 | NRPLMSCCF |
| 10035 | EFWWMNYMG |
| 10036 | TSQYSSVST |
| 10037 | MGTEWTQSY |
| 10038 | MWTEFDEYG |
| 10039 | KQLFMKFWN |
| 10040 | EFKCVSDCL |
| 10041 | KTIAQTYYG |
| 10042 | VQMQYERLF |
| 10043 | MDVQQIISG |
| 10044 | NPIKFQCAH |
| 10045 | NSVHVHASN |
| 10046 | CAEGQRALT |
| 10047 | QAIDMEFPL |
| 10048 | FNESGWPTW |
| 10049 | NARVMAKRE |
| 10050 | LNDPHCSQN |
| 10051 | YFADAWLKT |
| 10052 | NIHYHMRRN |
| 10053 | QMEVHWHMW |
| 10054 | REEWKNASN |
| 10055 | ISTKFGKEI |
| 10056 | AIHQESMIQ |
| 10057 | QEYHIYFPF |
| 10058 | TLTHCYSKD |
| 10059 | GCLTSECAY |
| 10060 | AWFNTYSVH |
| 10061 | GYGHTFRDA |
| 10062 | FIDEKQCHE |
| 10063 | HMSFCNFKV |
| 10064 | SVETARCSG |
| 10065 | SSEHYSASF |
| 10066 | LMKTSPSYW |
| 10067 | YGNSVTCTS |
| 10068 | KHITWNHCL |
| 10069 | DTSKTGKHV |
| 10070 | LTQYKALAQ |
| 10071 | TRIVEPDAN |
| 10072 | LADCMLNHH |
| 10073 | VPMWAHWQN |
| 10074 | SVTPDSITT |
| 10075 | QRNNLFVHD |
| 10076 | IHGIDCYEI |
| 10077 | GIDLMRQIN |
| 10078 | MKNQPTGAS |
| 10079 | QKSMLNSIQ |
| 10080 | ICNMDFRCA |
| 10081 | KTVLKMQDM |
| 10082 | RLGMNWKQI |
| 10083 | TPKSEGICC |
| 10084 | GVITVDAKG |
| 10085 | AIGPDRSNN |
| 10086 | EQEISDQCR |
| 10087 | MQYRGMSAS |
| 10088 | MISQMGLKN |
| 10089 | AFARQEYAN |
| 10090 | TYLHQPEDS |
| 10091 | TKLSMMGCE |
| 10092 | EELRYTCHT |
| 10093 | GSCCLNNET |
| 10094 | KKTLYEMAC |
| 10095 | RWGVMDLCN |
| 10096 | NVEDVGMIE |
| 10097 | QSWWHSDTC |
| 10098 | AADGYVAQD |
| 10099 | ITTPLHHHN |
| 10100 | NAQRLAVGM |
| 10101 | HVIGMGAGE |
| 10102 | RQYFEDNAG |
| 10103 | KSELNRFTY |
| 10104 | SALYRWGHV |
| 10105 | HRAGMSNIP |
| 10106 | MARQCHSFH |
| 10107 | ICKHLNVWP |
| 10108 | TNGKSSSYS |
| 10109 | HAVNFDCKK |
| 10110 | QISAWNEFF |
| 10111 | MMTPMAVEP |
| 10112 | KISTTWADK |
| 10113 | EVDQYYSLA |
| 10114 | GTMSFPTGC |
| 10115 | LMQWYNRCW |
| 10116 | GTGHIGNAT |
| 10117 | GGSYVSAPD |

TABLE 75
Sequences of the 581 to 589 Region in
AAV5 VP1 Capsid Polypeptide that Drive
Colon Tissue Tropism

| | 581-589 |
|---|---|
| SEQ ID NO | Sequence |
| 12118 | AQWYMWCWD |
| 12119 | ASHYHEKVW |
| 12120 | IANWRWGPD |
| 12121 | RVNGSTCRH |
| 12122 | YNRLMHQLW |
| 12123 | ETTMMIVSP |
| 12124 | STAKTGTTT |
| 12125 | LPPLKTEDN |
| 12126 | QTQICHSWM |
| 12127 | QSLWYWQQE |
| 12128 | KMGMHDMAM |
| 12129 | IRPQYAKMD |
| 12130 | MNANETHGA |
| 12131 | MSVAAGRGM |
| 12132 | QTMCITIHP |
| 12133 | CMEAQGECY |
| 12134 | FPQTMHAQA |
| 12135 | KPLASIGQY |
| 12136 | CCCQFSFWN |
| 12137 | DAELMWQIE |
| 12138 | ENTMWQSSQ |
| 12139 | KIDQTQWQR |
| 12140 | EQEISDQCR |
| 12141 | GKIAHDQLN |
| 12142 | MCFPYSCAA |
| 12143 | LSVQAQWDS |
| 12144 | MMNQCQVWN |
| 12145 | TQEACHHDP |
| 12146 | DFNGSNWLP |
| 12147 | HFEVRSVQR |
| 12148 | MAEQMERDF |
| 12149 | TGMAMSEQS |
| 12150 | PCCWEQSTN |
| 12151 | NWYTTWIDE |
| 12152 | CCKSKEVCA |
| 12153 | LESVKMDCN |
| 12154 | PHVSGEGKI |
| 12155 | TYDTRNHCL |
| 12156 | MAAITQNVA |
| 12157 | PDLWYEKSS |
| 12158 | AHAWLQHWA |
| 12159 | MAMWIADTH |
| 12160 | LCAVCGDCD |
| 12161 | RITQGESGM |
| 12162 | GNKSPDECF |
| 12163 | KNCIMLASN |
| 12164 | EKRLEGWVY |
| 12165 | LETMMRHGE |
| 12166 | EQPNMGDYG |
| 12167 | QSQWWDTCN |
| 12168 | LMKYQWIGN |
| 12169 | QISKPSVQC |
| 12170 | AIEHGAWKC |
| 12171 | SLTQYQWRG |
| 12172 | TIVVIPIKS |
| 12173 | CSMPQQCME |
| 12174 | QTAEWYCGA |
| 12175 | GNECFHNVE |
| 12176 | VGPLYAYYS |
| 12177 | WTLQVMKIN |
| 12178 | TSEPHCSYA |
| 12179 | AVHTADVCC |
| 12180 | QHSRAYMWY |
| 12181 | HMEQCWCNE |
| 12182 | AYAPSKNAM |
| 12183 | DNYWIVPFA |
| 12184 | QRSVFHQRS |
| 12185 | HEVMMCPAP |
| 12186 | KTMGIGGGP |
| 12187 | TLNSRHPEC |
| 12188 | PLNQEQIPQ |
| 12189 | AQCHDMWGH |
| 12190 | KACMSHQGR |
| 12191 | TTTNQFMFQ |
| 12192 | VLKSNLTGM |
| 12193 | MFMTNVNSF |
| 12194 | SSRMQMQGP |
| 12195 | RYPCCQSPF |
| 12196 | NARVMAKRE |
| 12197 | GLSMVLHFV |
| 12198 | VSVQSRAQL |
| 12199 | DMTLQSVSS |
| 12200 | MTDNNGPLF |
| 12201 | PLTIEVNCT |
| 12202 | YVDHWTLGD |
| 12203 | NQREWHGLA |
| 12204 | SECEVLCSL |
| 12205 | CAWHMKQGN |
| 12206 | ATGTYNLQE |
| 12207 | KCADLIIRS |
| 12208 | QSRCVDNTV |
| 12209 | ATMNLSWGL |
| 12210 | SVLHVQALA |
| 12211 | KMAGIGCWY |
| 12212 | YDYWMQAPT |
| 12213 | HSMMPGWPS |
| 12214 | MFWTTPSHW |
| 12215 | TKHCFYGGC |
| 12216 | AMNWWAAPQ |
| 12217 | AMNWWDAPH |
| 12218 | AMNWWDAPP |
| 12219 | AMNWWGAPQ |
| 12220 | CDRPDVNWF |
| 12221 | CDRPDVNWV |
| 12222 | CHSLHQVWA |
| 12223 | CHSLHQVWD |
| 12224 | CHSLHQVWG |
| 12225 | CHSLRQVWD |
| 12226 | CKTMKDIER |
| 12227 | DMQQNDPCC |
| 12228 | DVDFRAWAT |
| 12229 | DVHLFPTEQ |
| 12230 | ECHYRMFQI |
| 12231 | ECPYRMFQI |
| 12232 | EHHWGDIID |
| 12233 | ESAHECEHD |
| 12234 | ESAHECGHD |
| 12235 | ESAHEWEHD |
| 12236 | EYHYRMFQI |
| 12237 | FAVECVDCT |
| 12238 | FEVECVDCT |
| 12239 | FEVGCVDCT |
| 12240 | FLGFKRYGG |
| 12241 | FLSFERYGG |
| 12242 | FLSFKRYGC |
| 12243 | FLSFKRYGG |
| 12244 | FLSFKRYGS |
| 12245 | FLSFQRYGG |
| 12246 | FLSFRRYGG |
| 12247 | FNLTAHEDN |
| 12248 | GLEINMLNC |
| 12249 | HCAKFECMF |
| 12250 | IANTNPRGR |
| 12251 | IEAPKVNEY |
| 12252 | IEDPEVNEY |
| 12253 | IEDPKVNAY |
| 12254 | IEDPKVNEC |
| 12255 | IEDPKVNEH |
| 12256 | IEDPKVSEY |
| 12257 | IEDPRVNEY |
| 12258 | IEDPTVNEY |
| 12259 | IEDRKVNEY |
| 12260 | IGDPKVNEY |
| 12261 | IYTYQWDWI |
| 12262 | IYTYQWDWT |
| 12263 | IYTYQWGWT |
| 12264 | KCHYRMFQI |
| 12265 | KDTEGLEAP |
| 12266 | KDTEGLGAP |
| 12267 | LASQKRDCD |
| 12268 | LEDPKVNEY |
| 12269 | LINHWMHDN |
| 12270 | LINKWMHDN |
| 12271 | LINQWMHNN |
| 12272 | LLNQWMHDN |
| 12273 | LSSQKRDCA |
| 12274 | LSSQKRDCG |
| 12275 | LSSQKRDCN |
| 12276 | LSSQKRDGD |
| 12277 | LSSQRRDCD |
| 12278 | MCATQHCEA |
| 12279 | MCEQYWTED |
| 12280 | MCEQYWTGD |
| 12281 | MGEQYWTED |
| 12282 | MGRMELDGV |
| 12283 | MHMQQNQMM |
| 12284 | MHMRQNQMM |
| 12285 | MHMWQNQMM |
| 12286 | MRATQHCEA |
| 12287 | MSRMELDGV |
| 12288 | MSRMEQDGV |
| 12289 | MSRMGLDGV |
| 12290 | MSWMELDGV |
| 12291 | NDDPGQAVG |
| 12292 | NVHLFPNEQ |
| 12293 | NVHLFPTAQ |
| 12294 | NVHLFPTEQ |
| 12295 | NVHLFPTGQ |
| 12296 | NVHLFPTKQ |
| 12297 | PACYSDVLN |
| 12298 | PEFLGEVDA |
| 12299 | PEFPGAVDA |
| 12300 | PEFPGEGDA |
| 12301 | PEFPGEVDA |
| 12302 | PEFPGEVDS |
| 12303 | PEFPGEVDV |
| 12304 | PEFPGEVYA |
| 12305 | PGFPGEVDA |
| 12306 | PKFPGEVDA |
| 12307 | PQCLHHHWY |
| 12308 | PQCLHNHWY |
| 12309 | PQCLHSHWY |
| 12310 | PQCLRNHWY |
| 12311 | PQCLYNHWY |
| 12312 | QPVGLFAFA |
| 12313 | QSKSEGIHI |
| 12314 | QTDILDYAW |
| 12315 | QTDILDYDW |
| 12316 | QTDILDYYW |
| 12317 | RDRPDVNWV |
| 12318 | RLEINMLHC |
| 12319 | RLEINMLNC |
| 12320 | RLEINMRNC |
| 12321 | RLGINMLNC |
| 12322 | SCHQCDLCD |
| 12323 | SCHQCNLCD |
| 12324 | SCHQCSLCD |
| 12325 | SCIFRHWLT |
| 12326 | SCSPDAWET |
| 12327 | SCSPDDWGT |
| 12328 | SCSPDGWET |
| 12329 | SCSPGDWET |
| 12330 | SLQRWFCRT |
| 12331 | SLQRWVCRT |
| 12332 | STIHAIEQG |
| 12333 | SYHQCNLCD |
| 12334 | TMNWWDAPQ |
| 12335 | TSVQSVDRG |
| 12336 | TSVRSVDRD |
| 12337 | TSVRSVDRG |
| 12338 | VQCIHHDYH |
| 12339 | VQCIHHDYR |
| 12340 | VQCIHHGYR |
| 12341 | VQCIHHNYR |
| 12342 | VQCIHPDYR |
| 12343 | VQCIHRDYR |
| 12344 | VQCIRHDYR |
| 12345 | WKPPLSDAP |
| 12346 | WPSMQCAAH |
| 12347 | WPSMQCAAN |
| 12348 | WPSMQCAAR |
| 12349 | LSSQKRDCD |
| 12350 | IEDPKVNEY |
| 12351 | SCSPDDWET |
| 12352 | AMNWWDAPQ |
| 12353 | LINQWMHDN |
| 12354 | AAEQRFNPE |
| 12355 | AAILMSNAG |
| 12356 | ACMMMWPYV |
| 12357 | ADTGSTAVI |
| 12358 | AESVEDYHT |
| 12359 | AFELHYHHQ |
| 12360 | AFKFKMLGQ |
| 12361 | AFKWSHIHL |
| 12362 | AGVQYTWCG |
| 12363 | AIDQFSGCK |
| 12364 | AIEYRKYDR |
| 12365 | AIFMMSGPG |
| 12366 | AKWMITIGC |
| 12367 | ALDWCTRQA |
| 12368 | ALICVNVFG |
| 12369 | AMHTQGSLG |
| 12370 | AMTQEQMAD |
| 12371 | ANNSCYRPY |
| 12372 | APLPWWVVP |
| 12373 | ARRYNPELQ |
| 12374 | ARSPWMHMS |
| 12375 | ASDAKPAQL |
| 12376 | ASDDQRDSK |
| 12377 | ASHPCYSMH |
| 12378 | ATDIWCAFC |
| 12379 | ATNDSPHLA |
| 12380 | ATSWNTAIG |
| 12381 | ATYVYERFE |
| 12382 | AWCITYGLG |
| 12383 | AYERYDCYD |
| 12384 | AYTKMAAQG |
| 12385 | AYVAVASQW |
| 12386 | AYVPEFRDT |
| 12387 | CATRKDEMF |
| 12388 | CEECQEMLT |
| 12389 | CFRICVSEY |
| 12390 | CGEDMKIWQ |
| 12391 | CGGCLNLGT |
| 12392 | CGGTHGMMG |
| 12393 | CHVDRAMHH |
| 12394 | CHVSVPVDC |
| 12395 | CIANVDNFD |
| 12396 | CIEYTHRLA |
| 12397 | CILNTCSFC |
| 12398 | CIPPLSDKF |
| 12399 | CKAPAQDFR |
| 12400 | CKAPTRSYR |
| 12401 | CKSIGCMDE |
| 12402 | CNDMTAQYV |
| 12403 | CPDQYRAYP |
| 12404 | CQGFVKGQS |
| 12405 | CRINMERVS |
| 12406 | CTKKDNMDN |
| 12407 | CTNRHTPFI |
| 12408 | CWATEWQMP |
| 12409 | CWEQEWPFW |
| 12410 | CWQNKINHI |
| 12411 | CYVMRRIYC |
| 12412 | DADQAAKAC |
| 12413 | DAGMCEKED |
| 12414 | DANIASSGR |
| 12415 | DCMVFINYL |
| 12416 | DDCTNGSME |
| 12417 | DDVNNVAIA |
| 12418 | DDWQKLGVP |
| 12419 | DELYTGPCA |
| 12420 | DFNTKTSSL |
| 12421 | DGEFQAYWE |
| 12422 | DGGMIRGVS |
| 12423 | DGNKREWLS |
| 12424 | DGSPKNTSS |
| 12425 | DHIQMHRFN |
| 12426 | DINANMDKR |
| 12427 | DIQSNCSEA |
| 12428 | DISEQNRMC |
| 12429 | DISQHPIGS |
| 12430 | DIYLSNGTY |
| 12431 | DKCMLMDAS |
| 12432 | DNENCVQKT |
| 12433 | DQNEEGTKP |
| 12434 | DRIGYCNNW |
| 12435 | DRQGCNAPR |
| 12436 | DSMWRTKEL |
| 12437 | DSMYASSMP |
| 12438 | DSRQVKKFN |
| 12439 | DSTWLIAQQ |
| 12440 | DTGVYSSWE |
| 12441 | DTSADKSGW |
| 12442 | DTWISHWWC |
| 12443 | DVKSENHDW |
| 12444 | DVNVAASWV |
| 12445 | DVVQEFWAK |
| 12446 | DWHHEHNPN |
| 12447 | DWQRVKTMW |
| 12448 | DWSTIEWTA |
| 12449 | DYWSYPNYF |
| 12450 | DYYESWWMH |
| 12451 | EAHQSTLST |
| 12452 | EAKFLGGCW |
| 12453 | EANHYIEIQ |
| 12454 | EAPKVTVWG |
| 12455 | EASMKWWSN |
| 12456 | ECDHHEEHY |
| 12457 | ECQCCECQD |
| 12458 | ECRMRLDNS |
| 12459 | EDRNCSECL |
| 12460 | EEKPNYHTA |
| 12461 | EFDYYCLKT |
| 12462 | EFITKNWEH |
| 12463 | EHECPNSGM |
| 12464 | EHETTMAEA |
| 12465 | EICSHLCWA |
| 12466 | EIILGGYHN |
| 12467 | EKGWSEPGN |
| 12468 | EKQQIEIAS |
| 12469 | EMMSQSACA |
| 12470 | ENESSHTNP |
| 12471 | ENKVCVYMM |
| 12472 | EPYQKDCEL |
| 12473 | EQQAPDHVK |
| 12474 | ERGVMCHFW |
| 12475 | ESENPMGPY |
| 12476 | ESHMMRISE |
| 12477 | ETQTHYTSC |
| 12478 | ETWLMHHHV |
| 12479 | ETYIGGCCM |
| 12480 | EVGWIPPYM |
| 12481 | EVNYVKCND |
| 12482 | EVTNRTIPR |
| 12483 | EWKCSQNLT |
| 12484 | EWKYSQNLT |
| 12485 | EYNQCTFGM |
| 12486 | EYYQSVKMS |
| 12487 | FAQLHAVGN |
| 12488 | FASAMDVTL |
| 12489 | FDRHRWQPL |
| 12490 | FEHFDLSAS |
| 12491 | FLGNIKSYE |
| 12492 | FLQNVGFFN |
| 12493 | FMMAFKLDC |
| 12494 | FMQPIKQMD |
| 12495 | FNPWDKYGW |
| 12496 | FQPHIRDVG |
| 12497 | FQYRPAEHV |
| 12498 | FRVLQGNSV |
| 12499 | FTETTHCGH |
| 12500 | FTSGHGYME |
| 12501 | FVHKTEGED |
| 12502 | FVHTVTVNP |
| 12503 | FYFPIGMRP |
| 12504 | GDGRAETWQ |
| 12505 | GEDCPSAEE |
| 12506 | GELVRRLSQ |
| 12507 | GFCYPNKMI |
| 12508 | GFTQDPVGH |
| 12509 | GGDTGEYLF |
| 12510 | GHEPNKHFQ |
| 12511 | GHQVTEYMF |
| 12512 | GHYVIHKTN |
| 12513 | GIITPSMGS |
| 12514 | GMNPQLECP |
| 12515 | GQPINGKQE |
| 12516 | GSARQDGKP |
| 12517 | GSCLGISDM |
| 12518 | GSMQIAQEG |
| 12519 | GSMQSPTRR |
| 12520 | GSNSHLPLQ |
| 12521 | GSNSHLPLR |
| 12522 | GSTSKDSSM |
| 12523 | GTHPCGCPI |
| 12524 | GTSFNVHGA |
| 12525 | GVDHFFWVS |
| 12526 | GVHSAFYQT |
| 12527 | GVIWIHDEN |
| 12528 | GYNHVDSWS |
| 12529 | HCHLCSLRG |
| 12530 | HDERMWAMD |
| 12531 | HEPRTICTF |
| 12532 | HEQIGPEYR |
| 12533 | HFGYMHCSQ |
| 12534 | HNEHKVGYG |
| 12535 | HPLKIPIGK |
| 12536 | HPPSMIRVV |
| 12537 | HQNHPPAFD |
| 12538 | HSFEIHDEG |
| 12539 | HSNPRCVFF |
| 12540 | HTDQDRHAE |
| 12541 | HTSPCWGSY |
| 12542 | HVEMPAPGC |
| 12543 | HWIHMVNQQ |
| 12544 | ICFWTIAQW |
| 12545 | ICKPEGGFK |
| 12546 | IDNMPKCRP |
| 12547 | IDVHNGFDQ |
| 12548 | IDVRPHFAD |
| 12549 | IEMSEQPNC |
| 12550 | IETRMESMG |
| 12551 | IFSHWFAGW |
| 12552 | IGCLYTSNW |
| 12553 | IGDQAMECS |
| 12554 | IKCEPWVSG |
| 12555 | ILAQENRWA |
| 12556 | IMEWKNTMV |
| 12557 | IMMHRVMSD |
| 12558 | IMRDMVLPV |
| 12559 | IMTNYHYLD |
| 12560 | IMVKRTVNA |
| 12561 | INCKPSVQF |
| 12562 | INVDKDQFL |
| 12563 | IQCSTNSWE |
| 12564 | IQFVTWRFE |
| 12565 | IQGPSWHKN |
| 12566 | IQQVDMMCP |
| 12567 | IRWVEPMCQ |
| 12568 | ISPCPTVVA |
| 12569 | ISPNMQWYP |
| 12570 | IVFQGDTGT |
| 12571 | IVTVNCAGE |
| 12572 | IWCNGQMDD |
| 12573 | IYTYFIEWF |
| 12574 | KAFPIDLIA |
| 12575 | KDDHCAFWN |
| 12576 | KEGVDQEFF |
| 12577 | KEKQNAKCL |
| 12578 | KHHPTDADN |
| 12579 | KICMYHLID |
| 12580 | KIFWTTEFH |
| 12581 | KKTEEHHCQ |
| 12582 | KMMHLPQEE |
| 12583 | KMVQDEMWC |
| 12584 | KQEPMHCSA |
| 12585 | KSDQVWQGE |
| 12586 | KSTIFKMRH |
| 12587 | KTECFFSMH |
| 12588 | KTFPAADVK |
| 12589 | KVEFTATVM |
| 12590 | KVNIYSDTL |
| 12591 | LAERWEQNN |
| 12592 | LAHEPYWSP |
| 12593 | LALCDAVND |
| 12594 | LALHSQFCE |
| 12595 | LASPKQMVA |
| 12596 | LCAPYNREV |
| 12597 | LCGCCMMHY |
| 12598 | LCGNDKKCD |
| 12599 | LCHFTKGVY |
| 12600 | LDQGMYFHK |
| 12601 | LECWNKIAR |
| 12602 | LGYHVKDFM |
| 12603 | LIAFLVDRI |
| 12604 | LIMPLTTQM |
| 12605 | LMKMWTSMG |
| 12606 | LPDFWRTRN |
| 12607 | LPIFVQGDF |
| 12608 | LRAILDCGW |
| 12609 | LSATMVWAQ |
| 12610 | LSIQDNRGQ |
| 12611 | LSTSSQLTP |
| 12612 | LSVAVQESN |
| 12613 | LSWMACAQA |
| 12614 | LSYVMDTAA |
| 12615 | LTQFSCVSH |
| 12616 | LWVWWMKDH |
| 12617 | LYEEHHACW |
| 12618 | MAASGMLCY |
| 12619 | MADGRPRMP |
| 12620 | MATCYHDHW |
| 12621 | MCAQPCNLP |
| 12622 | MCVHKFLTG |
| 12623 | MDGPWMGKL |
| 12624 | MEHVPTRDQ |
| 12625 | MEMPLMDVI |
| 12626 | MGTQYQFTG |
| 12627 | MHAETDIQM |
| 12628 | MHFTSWPVD |
| 12629 | MKIMCENVR |
| 12630 | MNMEKLQID |
| 12631 | MNRIMMVAD |
| 12632 | MNSPRRECI |
| 12633 | MQIILEFPR |
| 12634 | MQTAHPVYL |
| 12635 | MRNHGDFDF |
| 12636 | MSILASVRV |
| 12637 | MSIRGNWGY |
| 12638 | MSKPCRVCE |
| 12639 | MTDPGMKRR |
| 12640 | MWVQRETGG |
| 12641 | NAAIWEHVH |
| 12642 | NACTKVKMT |
| 12643 | NAYNFTKDC |
| 12644 | NCENKQNVI |
| 12645 | NCPCHTDAY |
| 12646 | NCREHPEEG |
| 12647 | NDIECEMAS |
| 12648 | NEYEKLFPR |
| 12649 | NFMSMYGFE |
| 12650 | NHFPILIDH |
| 12651 | NHKLHTGPG |
| 12652 | NHWMGEKEC |
| 12653 | NIGFAIVDP |
| 12654 | NIGKERMEN |
| 12655 | NIGTHMGHT |
| 12656 | NIYVTRAHC |
| 12657 | NKSPTWTES |
| 12658 | NLHTPAGCM |
| 12659 | NLVCRESSC |
| 12660 | NPCIKASYF |
| 12661 | NPNGPLHYD |
| 12662 | NQMPKEKWD |
| 12663 | NRTGVTPST |
| 12664 | NRVDAMIHY |
| 12665 | NRWMMGMKP |
| 12666 | NSTEQEIIQ |
| 12667 | NTMTSAPVC |
| 12668 | NTNMAWKQF |
| 12669 | NVTELHNPK |
| 12670 | NVTETGPDA |
| 12671 | NYASEEFLT |
| 12672 | NYMGIDEQK |
| 12673 | NYSMPVDWS |
| 12674 | PCVEPNAAM |
| 12675 | PDARGMHFL |
| 12676 | PEHPRDWLM |
| 12677 | PHAGQGNKG |
| 12678 | PHGPITNPE |
| 12679 | PIAIKDQGW |
| 12680 | PIASMFNGP |
| 12681 | PKILFMFSF |
| 12682 | PLMIARYDP |
| 12683 | PMAPDWRNS |
| 12684 | PMTLPLECW |
| 12685 | PNEWYLWAC |
| 12686 | PPNMTSCDW |
| 12687 | PQDQSCAGQ |
| 12688 | PQHWKSVGA |
| 12689 | PTAMWLTHM |
| 12690 | PVATYNVSR |
| 12691 | PVEPSCQMK |
| 12692 | PVEVMQWKP |
| 12693 | PYEPQTINC |
| 12694 | PYVDKWANH |
| 12695 | QAAPVHAKG |
| 12696 | QAEATHAEF |
| 12697 | QATCTRYGH |
| 12698 | QCGCCKMKW |
| 12699 | QCIQIDMER |
| 12700 | QDDEYHMTP |
| 12701 | QEIAKKWSN |
| 12702 | QETGPKSST |
| 12703 | QFKDSRGAG |
| 12704 | QGDPVKFMS |
| 12705 | QGKAKQIQH |
| 12706 | QGTHSQNDD |
| 12707 | QIAQQPCMW |
| 12708 | QIDNVNQDP |
| 12709 | QITKKDNLM |
| 12710 | QKLVHKESF |
| 12711 | QNEKYGKTL |
| 12712 | QQGPLQRIM |
| 12713 | QQKRVCDQM |
| 12714 | QRPWHDLTY |
| 12715 | QSAQSHSHV |
| 12716 | QSNHEFRWF |
| 12717 | QVAVDQSPF |
| 12718 | QVYALSKHW |
| 12719 | QWDRCLVQV |
| 12720 | QWFVQGITL |
| 12721 | QYKVKERGM |
| 12722 | QYNSHFLYG |
| 12723 | RCDQHDDQW |
| 12724 | RCTDCSYFY |
| 12725 | RDQQIHHED |
| 12726 | REVKPMHVH |
| 12727 | RNDVCCEMQ |
| 12728 | RNLYENQGC |
| 12729 | RPCSKSHEF |
| 12730 | RPESEHPEG |
| 12731 | RPREGEWPG |
| 12732 | RSLHYGNSV |
| 12733 | RSPPCDSFP |
| 12734 | RTNDHPDKH |
| 12735 | RVDCETPYH |
| 12736 | RVGNERLHH |
| 12737 | RWYHYLLRN |
| 12738 | RYCLLDEIE |
| 12739 | SCKHDDMGK |
| 12740 | SCKWIQSNR |
| 12741 | SCLDHQDID |
| 12742 | SCNCKKNKI |
| 12743 | SDKGRQHYN |
| 12744 | SDNNAPEFQ |
| 12745 | SEGVYNQVY |
| 12746 | SFHVAQGDY |
| 12747 | SFIAPCDLS |
| 12748 | SGCTWDMSP |
| 12749 | SGPDTVFFT |
| 12750 | SHASGIECT |
| 12751 | SHESTNAGA |
| 12752 | SHMQDKNTT |
| 12753 | SHVEPVMGF |
| 12754 | SIRWFYTDC |
| 12755 | SIVNHNEAR |
| 12756 | SIWGCNVGG |
| 12757 | SKHEWDMHV |
| 12758 | SLDRFNQSG |
| 12759 | SLILMHEQT |
| 12760 | SLNWWFWGG |
| 12761 | SMGPLWDPL |
| 12762 | SMHTVINKG |
| 12763 | SNDCRNEFT |
| 12764 | SNSCEYQIM |
| 12765 | SPCFYPDCS |
| 12766 | SPICLQHDR |
| 12767 | SQKNSRHLS |
| 12768 | SSALITRKE |
| 12769 | SSSFWENKP |
| 12770 | SSSSHLAAD |
| 12771 | SSVCKTACW |
| 12772 | STARDLYLM |
| 12773 | STSHPDNSI |
| 12774 | SVFDVWCDQ |
| 12775 | SVMQGSSIQ |
| 12776 | SVWSTRMQK |
| 12777 | SWEILHDCH |
| 12778 | SWHWMTTQN |
| 12779 | SWYGNEEDQ |
| 12780 | SYHDHEQGI |
| 12781 | SYQLPILHS |
| 12782 | SYSYVNDEA |
| 12783 | TCKGMDNAI |
| 12784 | TCTEPIVEC |
| 12785 | TCVQMEHVK |
| 12786 | TCYWDNCWY |
| 12787 | TDRWTRVMS |
| 12788 | TEDQYNNIH |
| 12789 | TERWGWLPH |
| 12790 | TFWCAQLGV |
| 12791 | TFWIRMAAG |
| 12792 | TGLYYEMQE |
| 12793 | TGQNGWSKT |
| 12794 | TGVVDTAYS |
| 12795 | THSKSDCAY |
| 12796 | THTVRDSHI |
| 12797 | TIDRCNWHY |
| 12798 | TILHGMLLE |
| 12799 | TITHEQPGP |
| 12800 | TIVPYEYNQ |
| 12801 | TMDPCRKSC |
| 12802 | TMGVYSFDW |
| 12803 | TMMLVVLRG |
| 12804 | TNGIGGKKE |
| 12805 | TQLHWNDGR |
| 12806 | TQQALPLAC |
| 12807 | TSAPDRLSS |
| 12808 | TTEQIRDID |
| 12809 | TTVSCVCKG |
| 12810 | TVLRRCNYE |
| 12811 | TVYAWLLSM |
| 12812 | TWFVGQSTR |
| 12813 | TWQRTENNK |
| 12814 | TYLMNQFHM |
| 12815 | VCKMPMYQH |
| 12816 | VCRCTKYTA |
| 12817 | VDPNLQYPW |
| 12818 | VDWFTAPAP |
| 12819 | VFCNYQKKA |
| 12820 | VGSKWHVLG |
| 12821 | VGYSTVGEW |
| 12822 | VHHDLGESK |
| 12823 | VHHHGYPTS |
| 12824 | VHKEWHWQC |
| 12825 | VHMYPMTQV |
| 12826 | VKPNETDLH |
| 12827 | VLGFDHDPQ |
| 12828 | VLLMDGRTF |
| 12829 | VLRYVNSAG |
| 12830 | VMELMMHNH |
| 12831 | VPLVNDGRC |
| 12832 | VPTICMATY |
| 12833 | VQNEADMNP |
| 12834 | VRAMPFDPD |
| 12835 | VRNTEMDWF |
| 12836 | VSAPFCHIY |
| 12837 | VSMWYDRDD |
| 12838 | VTFMITMFH |
| 12839 | VVDHFFWVS |
| 12840 | VVITDGGQV |
| 12841 | VYMIFSGWQ |
| 12842 | WALAPELWY |
| 12843 | WAMDNMNRC |
| 12844 | WDHRYQTGH |
| 12845 | WDQCTMINQ |
| 12846 | WDSPQTNFT |
| 12847 | WFHTRHPQE |
| 12848 | WGPCKHPWQ |
| 12849 | WGVVKMVLC |
| 12850 | WHDKLRNNK |
| 12851 | WIDGIVDIC |
| 12852 | WINGPASRY |
| 12853 | WIQLNHQEV |
| 12854 | WITAFDSIG |
| 12855 | WITVKEFST |
| 12856 | WLISNSPQW |
| 12857 | WRCPPEQWW |
| 12858 | WVSADTYEH |
| 12859 | YASLWESGW |
| 12860 | YCMTVICSE |
| 12861 | YDECQKIFV |
| 12862 | YDGCMWMPF |
| 12863 | YDQKGGQCW |
| 12864 | YEDIIWNMH |
| 12865 | YENVGSKAW |
| 12866 | YGHRRDEAR |
| 12867 | YHSHLDAGQ |
| 12868 | YKLNCFARP |
| 12869 | YLDCPHTQF |
| 12870 | YPMAKNRGD |
| 12871 | YPRHWTLQH |
| 12872 | YQHVWSQER |
| 12873 | YQNPDAPAL |
| 12874 | YQQQLMMNE |
| 12875 | YSDAVYCSM |
| 12876 | YTDALGMEP |
| 12877 | YTPLPAVNG |
| 12878 | YWLCYPKLC |
| 12879 | EKLTADAAI |
| 12880 | INSESEWNP |
| 12881 | QTTEFALVQ |
| 12882 | SGLYSQCGG |
| 12883 | SDVHMFCML |
| 12884 | QSNLMEFHS |
| 12885 | EAQNIHDPH |
| 12886 | HTTLNYMSN |
| 12887 | AATPQKNQE |
| 12888 | AFCRGHYQA |
| 12889 | AGAPPLAVH |
| 12890 | AGWQMCFDS |
| 12891 | AGWQMCYGS |
| 12892 | AHPILQGWD |
| 12893 | AHYQQSTDR |
| 12894 | AIGHGAWKC |
| 12895 | AMCMLSHIH |
| 12896 | ANELSWHPR |
| 12897 | AQGCKFYNG |
| 12898 | ATVTRARSM |
| 12899 | CACHMKQGN |
| 12900 | CDSLSLLQN |
| 12901 | CFCQCGMPS |
| 12902 | CHHSGESHQ |
| 12903 | CKGRANWYM |
| 12904 | CKWLAIPCR |
| 12905 | CQVGLQNCW |
| 12906 | CTKMHTMIE |
| 12907 | CWALNGQLG |
| 12908 | DALPERRYF |
| 12909 | DAMQKISCV |
| 12910 | DFIYHHQAP |
| 12911 | DGVVTVTNL |
| 12912 | DHDLATEWE |
| 12913 | DKHHSRIDG |
| 12914 | DMNMYLGSQ |
| 12915 | DSKITQFNH |
| 12916 | DVETNNWRC |
| 12917 | DVHDSQMCY |
| 12918 | DYNDFNWYF |
| 12919 | ELGTYDGMG |
| 12920 | EMQMEERLP |
| 12921 | EPGRMHHSW |
| 12922 | EQCLAAAAE |
| 12923 | ESRPMIQPF |
| 12924 | EVCGHQKIQ |
| 12925 | EVHVTACGN |
| 12926 | EVVRIENPM |
| 12927 | EWFCYCGTC |
| 12928 | EYHKTQWGH |
| 12929 | FALIYHKDE |
| 12930 | FASPVLWGQ |
| 12931 | FEGFIERQH |
| 12932 | FGHRYDSAY |
| 12933 | FGNPMQTCP |
| 12934 | FGVNDMDSA |
| 12935 | FHCICWTHM |
| 12936 | FKYDCYQRN |
| 12937 | FLGCYCPYA |
| 12938 | FLRNTWLGP |
| 12939 | FMPYCVHSG |
| 12940 | FPSWMMKAP |
| 12941 | FSTAHMFGW |
| 12942 | FWCRPRLHD |
| 12943 | FYHHLPEEP |
| 12944 | GAGRMNCNV |
| 12945 | GANMMHMMN |
| 12946 | GCLISECAY |
| 12947 | GDKMGIYHC |
| 12948 | GEMPYCNRQ |
| 12949 | GFKVEQPYE |
| 12950 | GHTNCFAMS |
| 12951 | GIDEAKADY |
| 12952 | GKYWLQRSV |
| 12953 | GLFHPQFVQ |
| 12954 | GNISTFCWQ |
| 12955 | GQHHEETIY |
| 12956 | GQILFRSHT |
| 12957 | GQNPKMSAE |
| 12958 | GSCCLHIHH |
| 12959 | GTQHEKMYQ |
| 12960 | GTTQCTVHT |
| 12961 | GYDSSEAFE |
| 12962 | GYVTEGFDE |
| 12963 | HCPCKCTWL |
| 12964 | HERHATLMR |
| 12965 | HLLGNLEYM |
| 12966 | HNVCTMWAQ |
| 12967 | HQIQFCPCP |
| 12968 | HTQEELITP |
| 12969 | HTYCYWQSC |
| 12970 | IAAKVQNHD |
| 12971 | ICAAPNSAK |
| 12972 | IDHDNWDML |
| 12973 | IIGQTSIQD |
| 12974 | IKEHIPYWD |
| 12975 | IMNKAHYRF |
| 12976 | IQAGHQATM |
| 12977 | IVDMNTATK |
| 12978 | KCFTHAIVG |
| 12979 | KDWIPSYAL |
| 12980 | KEGCHVRMG |
| 12981 | KMQGGNGYG |
| 12982 | KNAYNMTIQ |
| 12983 | KPEILHFHD |
| 12984 | KTAENVWAM |
| 12985 | KTESIVKLQ |
| 12986 | KTIQHGTAD |
| 12987 | KYSDFCCPV |
| 12988 | KYTEALSCG |
| 12989 | LAMCALDNF |
| 12990 | LAMRALDNF |
| 12991 | LCSTEGMHT |
| 12992 | LCSTKGMHT |
| 12993 | LDTKRTFKK |
| 12994 | LGEATWQAV |
| 12995 | LMMKCCAMH |
| 12996 | LPDRDWHKW |
| 12997 | LQTKKDCPA |
| 12998 | LVVEKPSES |
| 12999 | MACKKAQDV |
| 13000 | MAKRVELVT |
| 13001 | MCHSFPTRP |
| 13002 | MEVEAEKPN |
| 13003 | MMKYYDAIG |
| 13004 | MMMPQMWSG |
| 13005 | MNRNVEMDH |
| 13006 | MTDKLYQED |
| 13007 | MTNWLMRGR |
| 13008 | MVNNWDWFY |
| 13009 | NCDFMVASI |
| 13010 | NCQPYGEPT |
| 13011 | NCSHYTQHY |
| 13012 | NGLLDFDPH |
| 13013 | NGLVDFDPH |
| 13014 | NIAPGGTIF |
| 13015 | NLEAMDCNY |
| 13016 | NMCTTFRLL |
| 13017 | NMKTKSAHK |
| 13018 | NNHRLNDAN |
| 13019 | NTMQSPAWV |
| 13020 | NVTPTDREG |
| 13021 | PCGRTCFNG |
| 13022 | PMMSFIFHH |
| 13023 | PQTAAQKSD |
| 13024 | PTEVTVSDM |
| 13025 | PTQWAMGDY |
| 13026 | QAAYCRCTF |
| 13027 | QATHDLMHQ |
| 13028 | QATLHHCTN |
| 13029 | QCLLWNDCQ |
| 13030 | QGQEKEWYY |
| 13031 | QHMELQEHD |
| 13032 | QPCIPVQIA |
| 13033 | QQLYFTTDK |
| 13034 | QVIPVTYRK |
| 13035 | QVKMGVIDQ |
| 13036 | QVLATMNNK |
| 13037 | QWRQMDRMQ |
| 13038 | RDDQPPMVW |
| 13039 | RDFVAEQQP |
| 13040 | REERYFKDE |
| 13041 | RIEMHQSVT |
| 13042 | RKAEQEDVY |
| 13043 | RLVTYPHTA |
| 13044 | RNATKIKLT |
| 13045 | RNCLTDAWD |
| 13046 | RSDQECSKA |
| 13047 | RTCKTIGYY |
| 13048 | RTEPTFVDP |
| 13049 | RTYQYEYEV |
| 13050 | SCWRETMKE |
| 13051 | SLENEARRC |
| 13052 | SLHIHGNGP |
| 13053 | SMGMCELPI |
| 13054 | SNAHMQVGC |
| 13055 | SNAYHAWKN |
| 13056 | SQDLFCNAA |
| 13057 | SSGWDHGNL |
| 13058 | SSTWQNVDK |
| 13059 | STHPLNLYH |
| 13060 | SVQPFLHDG |
| 13061 | TADRQLVHS |
| 13062 | TCGKMHGPN |
| 13063 | TLWDNTNTP |
| 13064 | TPSWFNVGA |
| 13065 | TRGHHCAED |
| 13066 | TVTCPEVRF |
| 13067 | VNQLHIHVQ |
| 13068 | VPWYEGPYE |
| 13069 | VRFEMMSCT |
| 13070 | VTKCGWMLQ |
| 13071 | WDNSKGYNV |
| 13072 | WNNEDLKVW |
| 13073 | YATQMRHLE |
| 13074 | YCLMDKEDT |
| 13075 | YDGKTFLHW |
| 13076 | YFDCSRAHP |
| 13077 | YGTKCERCY |
| 13078 | YGTKSERCY |
| 13079 | YHDLFFAWY |
| 13080 | YHTNNMEPW |
| 13081 | YMHVPPSIH |
| 13082 | YMWIYPPDQ |
| 13083 | YNVCTMWAQ |
| 13084 | YTQLYCSHL |
| 13085 | YVPCERCEW |
| 13086 | YVQSYWNES |
| 13087 | YWERHCMTI |
| 13088 | YYGWLLVMV |
| 13089 | AGWQMCYDS |
| 13090 | VNCNKRHNW |
| 13091 | PYCSIKQAS |
| 13092 | CQTHWEKGD |
| 13093 | EMISNNCHP |
| 13094 | RQRYMGMQF |
| 13095 | SYLFSKHSE |
| 13096 | KAISLHAHG |
| 13097 | GIGEMQCTE |
| 13098 | ILERYQHMD |
| 13099 | IQKDEMPVC |
| 13100 | GTQFWMSVE |
| 13101 | QMSWGKYHQ |
| 13102 | KDYWAGYQM |
| 13103 | ICDLYSQER |
| 13104 | EEAPGFICF |
| 13105 | DLPNDMGAV |
| 13106 | GMHATELWL |
| 13107 | LMHPQMTMC |
| 13108 | AAKKGRDLH |
| 13109 | RHIYRKGWR |
| 13110 | GEFADNPWN |
| 13111 | ERQMKLRCA |
| 13112 | TDSSDQLQI |
| 13113 | YNMVHGDEP |
| 13114 | NIKMLVHCG |
| 13115 | MKGYFVHHS |
| 13116 | MLRTQIHCA |
| 13117 | CKDNTFVSY |

TABLE 76
Sequences of the 581 to 589 Region in
AAV5 VP1 Capsid Polypeptide that Drive
Heart Tissue Tropism

| | 581-589 |
|---|---|
| SEQ ID NO | Sequence |
| 15118 | ADIDQIMCQ |
| 15119 | ADIDQIVCQ |
| 15120 | ADIDQVMCQ |
| 15121 | AHKYDWEYL |
| 15122 | ARSDWRCMC |
| 15123 | AWCEGNCLE |
| 15124 | CCCIMSVMC |
| 15125 | CCCIMSVMG |
| 15126 | CSRIMALSN |
| 15127 | DNRTEGYFT |
| 15128 | ECHLLSDYD |
| 15129 | EDWHGWNEH |
| 15130 | ELYMQMHWP |
| 15131 | ENRLRLAHE |
| 15132 | EQTMFNSPP |
| 15133 | FAMVVTTEY |
| 15134 | FFPHDGTIV |
| 15135 | FYQMFHASG |
| 15136 | GFPPRWKIP |
| 15137 | GHCCSNGPT |
| 15138 | GKQQCPAQT |
| 15139 | GKQQCPTQT |
| 15140 | GNRTEGYFT |
| 15141 | HDCALYGRF |
| 15142 | HLNPMTGSN |
| 15143 | HNVMCDACC |
| 15144 | ICKYPMIPH |
| 15145 | ICKYPMISH |
| 15146 | ICMYPMIPH |
| 15147 | IDEFWYGPF |
| 15148 | IDPRRNNDL |
| 15149 | KINPSMPMD |
| 15150 | KPDTSVGIG |
| 15151 | KPDTSVGIV |
| 15152 | KWPCHWHHE |
| 15153 | KWPCHWHHG |
| 15154 | LCINKYFNY |
| 15155 | LEPKMKLAD |
| 15156 | LEPKMKLAE |
| 15157 | LVCEVACKD |
| 15158 | MFTQNAGKI |
| 15159 | MSCPKFHIV |
| 15160 | NEDSLWNMH |
| 15161 | NEDSPWNMH |
| 15162 | NEDSPWSMH |
| 15163 | NNRCKDQTE |
| 15164 | PFTQHVAYI |
| 15165 | PFTQHVAYS |
| 15166 | PQQFAWNND |
| 15167 | PTAIQAYMR |
| 15168 | QHVSTEPPY |
| 15169 | QIADGWGWE |
| 15170 | QMCATMNHE |
| 15171 | QMQHCMDYL |
| 15172 | QVGWKECVM |
| 15173 | RCQQFHGQI |
| 15174 | RDINIDPCC |
| 15175 | SAMVVTTEY |
| 15176 | SFTQHVAYS |
| 15177 | SKQQCPAQT |
| 15178 | SNRTEGYFT |
| 15179 | TDIDQIMCQ |
| 15180 | TDRHDGVFA |
| 15181 | TEYQNARTM |
| 15182 | TITCEADNW |
| 15183 | VFRNRWLEW |
| 15184 | VIEVEQACT |
| 15185 | VIKVEQACT |
| 15186 | VPNPKNFGW |
| 15187 | VSVVQNEAL |
| 15188 | YCSIIGRSC |
| 15189 | YFALCPPYT |
| 15190 | YHAFYWQPY |
| 15191 | AADHWVCQF |
| 15192 | AAFQCAKQG |
| 15193 | AAGMHWNAA |
| 15194 | AAHMGHGWP |
| 15195 | AANQEQECL |
| 15196 | AAPCPDRFV |
| 15197 | AASDSNRIH |
| 15198 | ACCDKTRPE |
| 15199 | ACMFWHRCG |
| 15200 | ADGRGMDCP |
| 15201 | AEENWRQCA |
| 15202 | AEHDLKIVS |
| 15203 | AEVEQYAEA |
| 15204 | AFDPTFIDT |
| 15205 | AFEAFDYDH |
| 15206 | AFFLTPAEP |
| 15207 | AFGNLEVHM |
| 15208 | AGAPLIHQQ |
| 15209 | AGAQQYWAD |
| 15210 | AGECQLLMT |
| 15211 | AHNCNGHVP |
| 15212 | AHNWPAMYT |
| 15213 | AHNYCGYYV |
| 15214 | AHQPDQYCK |
| 15215 | AIAHCVQIE |
| 15216 | AIEGLTGRD |
| 15217 | AIEYMNMGF |
| 15218 | AIKDMRLAL |
| 15219 | AISSHWQWP |
| 15220 | AKNEECDCN |
| 15221 | AKVEMEEWN |
| 15222 | ALAWQMRAQ |
| 15223 | AMNDELKMQ |
| 15224 | AMVIIYTDF |
| 15225 | AMVLMFLYQ |
| 15226 | ANNQHRARA |
| 15227 | APFMSEDCS |
| 15228 | APLMTAFDP |
| 15229 | APMSQWHTV |
| 15230 | APNGYTMFY |
| 15231 | APSAHHTHL |
| 15232 | APYWDCFDR |
| 15233 | AQGPMSVQL |
| 15234 | ARPIFPWIV |
| 15235 | ASTVVYAEM |
| 15236 | ASWLADNLC |
| 15237 | ATAFRHRIN |
| 15238 | ATARLNWDA |
| 15239 | ATDQYWFPT |
| 15240 | ATEIKGSHS |
| 15241 | ATESKWDQD |
| 15242 | ATHIHYHGL |
| 15243 | ATIEGDGVL |
| 15244 | ATIRVITIG |
| 15245 | ATNYVVRHT |
| 15246 | ATSVMAWWD |
| 15247 | ATWRICSSG |
| 15248 | AVDFCQTYD |
| 15249 | AVHAAYCYW |
| 15250 | AVHAGPQAI |
| 15251 | AVKPREAWM |
| 15252 | AVMYNDTYD |
| 15253 | AVPCQLSGA |
| 15254 | AVQERFLQN |
| 15255 | AVQSQTKNM |
| 15256 | AVQWVEHEK |
| 15257 | AVRCDPYRF |
| 15258 | AVSTTVDWY |
| 15259 | AVTTPDVYV |
| 15260 | AVVAMNHHT |
| 15261 | AWGEMNFSV |
| 15262 | AWMPFIDSG |
| 15263 | AWPDIMWAS |
| 15264 | AWRGSKMSN |
| 15265 | AWSVQGPYG |
| 15266 | AWVDQEIPA |
| 15267 | AWVGAEHAG |
| 15268 | AYEHITDGR |
| 15269 | AYTDTNAGY |
| 15270 | AYVEKMRFD |
| 15271 | AYYHEMAVP |
| 15272 | CAAMQQGRD |
| 15273 | CAEAIIKNF |
| 15274 | CAHSNKGGA |
| 15275 | CAMQPCLAV |
| 15276 | CAVDKCMPH |
| 15277 | CCSSFRDMV |
| 15278 | CCWLANGGG |
| 15279 | CDMLTRMQY |
| 15280 | CDPWSWTQI |
| 15281 | CDSTRFCCV |
| 15282 | CEVARWLDY |
| 15283 | CEWKHHAKF |
| 15284 | CFHDPYEKQ |
| 15285 | CGCYSPFLE |
| 15286 | CGSDCNPAY |
| 15287 | CGYQLFECF |
| 15288 | CHNEDLCVW |
| 15289 | CHTMQDIKD |
| 15290 | CIGCPNEWC |
| 15291 | CIQHIAESE |
| 15292 | CISEMDDQR |
| 15293 | CISFSNKWV |
| 15294 | CKAAGPWTD |
| 15295 | CKDSFASVW |
| 15296 | CKPRLCEVP |
| 15297 | CKYMIHPDC |
| 15298 | CLYPLQLEW |
| 15299 | CMDQYRIQF |
| 15300 | CNACQAIEC |
| 15301 | CNKAVQKTS |
| 15302 | CNMPQITGA |
| 15303 | CNPAKSKDG |
| 15304 | CPCKGRRLG |
| 15305 | CPCLYCNLA |
| 15306 | CPEFGFICE |
| 15307 | CQIPKKIGM |
| 15308 | CQTAFYVGH |
| 15309 | CRDRDMAHQ |
| 15310 | CRLKKYYLK |
| 15311 | CSPRKSHFC |
| 15312 | CSRFMDSQW |
| 15313 | CSVDKEDMD |
| 15314 | CTKLSGLNA |
| 15315 | CTKWQRTLG |
| 15316 | CTLEFPSFL |
| 15317 | CVGIMTNGK |
| 15318 | CVGWAMGLG |
| 15319 | CVHKQGENE |
| 15320 | CVIQSNTHV |
| 15321 | CVQPTYADH |
| 15322 | CVTLEMQQG |
| 15323 | CWAMDWGDT |
| 15324 | CWQFTRCAS |
| 15325 | CYYHTCLGG |
| 15326 | DACFYGNFE |
| 15327 | DAENCMLGW |
| 15328 | DAGYCQSGV |
| 15329 | DALSFQSSA |
| 15330 | DANRKDLRG |
| 15331 | DAQAGWRFN |
| 15332 | DASSGFTWT |
| 15333 | DATDWKCAW |
| 15334 | DAVKAEVMW |
| 15335 | DAWAKGYEW |
| 15336 | DAWSANEAW |
| 15337 | DAYWMQWWL |
| 15338 | DCASFPAVD |
| 15339 | DCQGWTAWD |
| 15340 | DCVKAIQGW |
| 15341 | DCVQMNVNV |
| 15342 | DDCWQDRHV |
| 15343 | DDVWKELDV |
| 15344 | DEAYRMPLQ |
| 15345 | DEGAAAKAI |
| 15346 | DEGTSQTKT |
| 15347 | DEHDGCKKS |
| 15348 | DEMYVSSQF |
| 15349 | DFAQRNSAC |
| 15350 | DFGMKNYLA |
| 15351 | DFKFEMTDR |
| 15352 | DFLYSPIGI |
| 15353 | DFSTPYQFG |
| 15354 | DGCTSKDNQ |
| 15355 | DGIHSLTGW |
| 15356 | DGNQLCRLT |
| 15357 | DGNYIRVDV |
| 15358 | DGPCAQYRG |
| 15359 | DGPMFATDI |
| 15360 | DHHGAGYNR |
| 15361 | DHISDYQHY |
| 15362 | DIILRIRLK |
| 15363 | DIKAILSKD |
| 15364 | DILGVHLGS |
| 15365 | DIQWQHEWM |
| 15366 | DISCNLQFL |
| 15367 | DKMLKTDLM |
| 15368 | DKNSCCLNP |
| 15369 | DKPVGRYGH |
| 15370 | DKQFVEDNQ |
| 15371 | DKRFQKSYS |
| 15372 | DLMMHYEHK |
| 15373 | DLQMSVNTG |
| 15374 | DLSQMNKNY |
| 15375 | DMGEVQVGI |
| 15376 | DMNPDACVR |
| 15377 | DMQVLNTDD |
| 15378 | DMSEVDKVF |
| 15379 | DMTIAAWAC |
| 15380 | DMTKVTPFL |
| 15381 | DNACVHCAE |
| 15382 | DNCWLNDSA |
| 15383 | DNEPNNRKS |
| 15384 | DNISWSAWD |
| 15385 | DPHQGIAGE |
| 15386 | DPPIVQQWH |
| 15387 | DQKWDTLKP |
| 15388 | DQRRKTNIR |
| 15389 | DQTTLRLMG |
| 15390 | DQYWAWADS |
| 15391 | DRLEDKESQ |
| 15392 | DRMQFFVRN |
| 15393 | DRMTCDTDR |
| 15394 | DSEWTIDEH |
| 15395 | DSGEVRTAS |
| 15396 | DSKVFEWKC |
| 15397 | DSQLTNACG |
| 15398 | DTELVHNGW |
| 15399 | DTLSSDAFL |
| 15400 | DTMCYAWCK |
| 15401 | DTQPWVPCG |
| 15402 | DTRESGFWV |
| 15403 | DTRIKMMLP |
| 15404 | DVAKKNMDG |
| 15405 | DVASFGHEI |
| 15406 | DVCMLVDLP |
| 15407 | DVCMSNELT |
| 15408 | DVDCCEFCT |
| 15409 | DVDFHWQGM |
| 15410 | DVDHKWQYD |
| 15411 | DVDPMPCFY |
| 15412 | DVGIMDYWS |
| 15413 | DVLQHQGAL |
| 15414 | DVMMCFHWR |
| 15415 | DVPDKPWYH |
| 15416 | DVQLDQEWD |
| 15417 | DVQRKTSGE |
| 15418 | DVRFISEQG |
| 15419 | DVRPPAVNE |
| 15420 | DWPCDDFRK |
| 15421 | DYLSFCWKD |
| 15422 | DYNKDPKLV |
| 15423 | EACMHRTVL |
| 15424 | EADFVGPNS |
| 15425 | EAEEHCTFW |
| 15426 | EAEEYEPWK |
| 15427 | EAFQEDAHP |
| 15428 | EANSEYNVY |
| 15429 | EATKSVETR |
| 15430 | EAVTARVQH |
| 15431 | ECDDYDCHY |
| 15432 | ECTRSGKWG |
| 15433 | EDHVMTKKD |
| 15434 | EDIEKNTFE |
| 15435 | EDKQAQTCQ |
| 15436 | EDRSSCVWM |
| 15437 | EEEPLSEMA |
| 15438 | EEKMSVDRK |
| 15439 | EETVSKRKK |
| 15440 | EFIFHIHDQ |
| 15441 | EFMQSFFIE |
| 15442 | EGEDYMDIE |
| 15443 | EGNQFWMFS |
| 15444 | EGQTSNIWE |
| 15445 | EGVTTLQSS |
| 15446 | EHDTMEGFG |
| 15447 | EHGQEEMIM |
| 15448 | EHTAMWMHG |
| 15449 | EICNKGQPD |
| 15450 | EIEGVKANV |
| 15451 | EIGGNPIYD |
| 15452 | EIGYCMTCD |
| 15453 | EIKLPMEQD |
| 15454 | EIKPMVASH |
| 15455 | EILNHTRLW |
| 15456 | EIMDTKQCS |
| 15457 | EIMTMEFQD |
| 15458 | EINTPFFMS |
| 15459 | EINVTQTHQ |
| 15460 | EIQQMDDAP |
| 15461 | EIVATYGQN |
| 15462 | EIVELATQY |
| 15463 | EKHAFSYSF |
| 15464 | EKHMTMAGI |
| 15465 | EKMAREWQT |
| 15466 | EKMWPLNWG |
| 15467 | EKVMQMHMQ |
| 15468 | ELWKAMPPE |
| 15469 | EMEINPTVE |
| 15470 | EMHMTNTGW |
| 15471 | EMTKYLNHA |
| 15472 | ENHCWSAKE |
| 15473 | ENNAERWMF |
| 15474 | ENVQCELMQ |
| 15475 | ENWTWKPEE |
| 15476 | EPMYFIVCS |
| 15477 | EPVNFNIQV |
| 15478 | EQDGHNINE |
| 15479 | EQEQFHICG |
| 15480 | EQMLWNQIS |
| 15481 | EQQWNSVNE |
| 15482 | ERIMHLHES |
| 15483 | ERMPLTCMS |
| 15484 | ERSEHPCMF |
| 15485 | ESCWPQVTG |
| 15486 | ESEPIVYGW |
| 15487 | ESHNTTEGH |
| 15488 | ESLENPNDR |
| 15489 | ETDNFSDCA |
| 15490 | ETFVTCTGA |
| 15491 | ETHHGQMFC |
| 15492 | ETHMYERRE |
| 15493 | ETHVVQNDN |
| 15494 | ETILAPSGT |
| 15495 | ETLMTVKKC |
| 15496 | ETVRQGNPE |
| 15497 | EVARKVQNE |
| 15498 | EVAWMGRQN |
| 15499 | EVDPWGHME |
| 15500 | EVHVNWKDE |
| 15501 | EVIDHGHME |
| 15502 | EVLEWRHYQ |
| 15503 | EVNISGEYV |
| 15504 | EVTEPVKAI |
| 15505 | EVTKHLVAC |
| 15506 | EVWTPCFPI |
| 15507 | EWCNEWCGP |
| 15508 | EWDEWLISF |
| 15509 | EWIHLYCCF |
| 15510 | EWIPYAVGN |
| 15511 | EYDYIFNHH |
| 15512 | EYEADTITC |
| 15513 | EYMGFRCDR |
| 15514 | EYMSIMMNH |
| 15515 | EYNPTNHIL |
| 15516 | EYPAEFAQC |
| 15517 | EYQQLARHW |
| 15518 | FACMPNEFL |
| 15519 | FAFDTQMVF |
| 15520 | FAPPTHTLE |
| 15521 | FCHNIFTEC |
| 15522 | FDHSERQYH |
| 15523 | FEKTRIAAW |
| 15524 | FEMGQKSLS |
| 15525 | FFEFRPSTK |
| 15526 | FGMVMPMWA |
| 15527 | FGPDSTHLC |
| 15528 | FGQLLMTDG |
| 15529 | FGWYYFWDP |
| 15530 | FHSCSRMGL |
| 15531 | FIDTACSHG |
| 15532 | FIGDTYAGG |
| 15533 | FIGMQSALE |
| 15534 | FITMRQEGP |
| 15535 | FIVILNNSD |
| 15536 | FNPDTDQDH |
| 15537 | FPFGTMHGS |
| 15538 | FPFLTKSPK |
| 15539 | FPQLPQNEA |
| 15540 | FSMDKQQFQ |
| 15541 | FTCEYTYAY |
| 15542 | FTELSMVQD |
| 15543 | FTKQADELP |
| 15544 | FTNFEYSME |
| 15545 | FTQGGEVYA |
| 15546 | FTSWMDVQD |
| 15547 | FTTQPSVAW |
| 15548 | FVPQVWQIP |
| 15549 | FVSSYYAIH |
| 15550 | FVSTESVWN |
| 15551 | FYIMMEKML |
| 15552 | GACCKQIGE |
| 15553 | GAKWQAQPD |
| 15554 | GATQHECWC |
| 15555 | GAYDTVWGW |
| 15556 | GCATWHNLY |
| 15557 | GCMLRSVTG |
| 15558 | GCNLLSVSN |
| 15559 | GDAFSCVFP |
| 15560 | GDDPMFFEK |
| 15561 | GEHTGMMLY |
| 15562 | GFMAVVQSW |
| 15563 | GGRCYFWED |
| 15564 | GGRLYKPEW |
| 15565 | GHASWAMWY |
| 15566 | GHGHKWIYS |
| 15567 | GHMYLLDME |
| 15568 | GHTCPCKWD |
| 15569 | GIDTDDVGH |
| 15570 | GIGMFCVTN |
| 15571 | GKARRCATW |
| 15572 | GKDVKQCCD |
| 15573 | GKLGLDCQD |
| 15574 | GKVVDAHMG |
| 15575 | GLQNKGYPT |
| 15576 | GLSIEISTP |
| 15577 | GLSLHKSDH |
| 15578 | GMADMVHGP |
| 15579 | GMASHSHYI |
| 15580 | GMQTIEQFN |
| 15581 | GNEFIECRQ |
| 15582 | GNHWGWSIS |
| 15583 | GPEMTVAQT |
| 15584 | GQTPCSSWG |
| 15585 | GQVDNMMAT |
| 15586 | GRELEIEPQ |
| 15587 | GSDRATGKE |
| 15588 | GSEFCILNE |
| 15589 | GSKPSSKQS |
| 15590 | GSSHFNDLG |
| 15591 | GTCHGPVLM |
| 15592 | GTCQVQLHS |
| 15593 | GTEIKTLHG |
| 15594 | GTEVIGLNT |
| 15595 | GVFLGYAQR |
| 15596 | GVISDGKYA |
| 15597 | GVKDFDVCT |
| 15598 | GVMVNPCNM |
| 15599 | GVSAEGKAQ |
| 15600 | GVSGMQITG |
| 15601 | GVTMNTECE |
| 15602 | GVVATDCEI |
| 15603 | GVYMESAVR |
| 15604 | GWETPLVWF |
| 15605 | GWWNHYLFW |
| 15606 | GYIYHKMYT |
| 15607 | HAAETEIHQ |
| 15608 | HCDTHVNLE |
| 15609 | HCKSGQQML |
| 15610 | HCTCYDAIE |
| 15611 | HDKWKWSDR |
| 15612 | HENYYREGA |
| 15613 | HFLQMEHGH |
| 15614 | HFMYDMNFQ |
| 15615 | HILKHVHTP |
| 15616 | HISFHTIPG |
| 15617 | HITGEYIAP |
| 15618 | HKESSEGAT |
| 15619 | HLNNCHWRR |
| 15620 | HMGQMYWHM |
| 15621 | HMVDINILA |
| 15622 | HNGVMMHSH |
| 15623 | HNKACITAL |
| 15624 | HPNNFNRAH |
| 15625 | HPVHYNMCS |
| 15626 | HSCTMMGGD |
| 15627 | HSMGIFSNL |
| 15628 | HSMVQWNST |
| 15629 | HVEMANAGK |
| 15630 | HVIPHNRSE |
| 15631 | HVMPWLCQW |
| 15632 | HVMTLVDYD |
| 15633 | HVNTEQKYY |
| 15634 | HWNCNCRNA |
| 15635 | HWPQRLNEH |
| 15636 | HWSWFASFV |
| 15637 | HWTNQDSNK |
| 15638 | HYHQYTRLP |
| 15639 | IADVIHSKW |
| 15640 | IAETLIGVL |
| 15641 | IAHGASAYL |
| 15642 | IAYSLLANY |
| 15643 | ICMFEEEQW |
| 15644 | ICPPFQRQV |
| 15645 | ICPYAEVSD |
| 15646 | ICSNPWNKE |
| 15647 | IDCQGKCDW |
| 15648 | IEDDPLHAE |
| 15649 | IEHRFMIAG |
| 15650 | IEPIHNWKK |
| 15651 | IESVTHLCD |
| 15652 | IGELDEACD |
| 15653 | IGFMDNTSC |
| 15654 | IGIPIHMWD |
| 15655 | IGQSIVTMQ |
| 15656 | IHMFRMWPE |
| 15657 | IHPQIAEGM |
| 15658 | IIDVFPRHT |
| 15659 | IIFFLCRDP |
| 15660 | IKQPANDTD |
| 15661 | IKYGMRHTN |
| 15662 | ILVMVWLGQ |
| 15663 | IMRWHYGMV |
| 15664 | INASSCDYW |
| 15665 | INRNYWDFI |
| 15666 | INSELPVDV |
| 15667 | INYSSVATV |
| 15668 | IPQPTMMQD |
| 15669 | IQEYIQDAD |
| 15670 | IRESWIGQF |
| 15671 | IRYVTAYYK |
| 15672 | ISEIMPSNP |
| 15673 | ISMYQSTQV |
| 15674 | ISNWRCSQK |
| 15675 | ITCSNEKDF |
| 15676 | ITENRGYYH |
| 15677 | ITSNELPME |
| 15678 | IVEFMHCDD |
| 15679 | IVEMYAKNN |
| 15680 | IVHDEPRHN |
| 15681 | IVHDIWGQA |
| 15682 | IVKYRAYPG |
| 15683 | IVNKNCSQP |
| 15684 | IVPFGCGGF |
| 15685 | IWKCLNTQN |
| 15686 | IWTCCKGGG |
| 15687 | IWVDYEITR |
| 15688 | IYEARTIVR |
| 15689 | KALPGMSAG |
| 15690 | KAPGVCTRS |
| 15691 | KCDMKRGQF |
| 15692 | KCELMSNQT |
| 15693 | KCWYKFITN |
| 15694 | KDALFDFPC |
| 15695 | KDDGMCYGR |
| 15696 | KDEDWNWCD |
| 15697 | KDFWAMSMV |
| 15698 | KDIGKAAGY |
| 15699 | KDVMFREMH |
| 15700 | KECEQLMFP |
| 15701 | KFDHDSEFY |
| 15702 | KFEPRYGDC |
| 15703 | KGIHEPISC |
| 15704 | KHDSDWMWY |
| 15705 | KHIQDARWF |
| 15706 | KHWREMITV |
| 15707 | KIDDDSRVY |
| 15708 | KIQYDDFDG |
| 15709 | KIVDIDWWA |
| 15710 | KLMWFASTD |
| 15711 | KLSNDRFIY |
| 15712 | KMQTVMRAL |
| 15713 | KNDMLKKFG |
| 15714 | KPIPQTVWL |
| 15715 | KPQAHYYTI |
| 15716 | KQLLTRMYM |
| 15717 | KQPEPTPLA |
| 15718 | KRMQEMTCR |
| 15719 | KSASPTQPY |
| 15720 | KSPAIHTEE |
| 15721 | KSSGVWMCC |
| 15722 | KTEWYDMKN |
| 15723 | KVEENLRSM |
| 15724 | KVIPEDQNC |
| 15725 | KWDSTCYRW |
| 15726 | KWTEFDRCD |
| 15727 | KYEEQYHFH |
| 15728 | KYQPDGWCY |
| 15729 | LAAGVWDQE |
| 15730 | LACKREAMW |
| 15731 | LAVCKTHEP |
| 15732 | LCHPGEHWQ |
| 15733 | LCVTGFDTH |
| 15734 | LDESWSVAG |
| 15735 | LDPNRCMDQ |
| 15736 | LGFWETQIM |
| 15737 | LGHMQASTP |
| 15738 | LGNQYWWHF |
| 15739 | LHEIHLETN |
| 15740 | LHLPQLLHD |
| 15741 | LHSAWMHLA |
| 15742 | LIAHWIHSF |
| 15743 | LIAPYFGQL |
| 15744 | LICTMSDMN |
| 15745 | LIDHIQNQH |
| 15746 | LKEMTWINW |
| 15747 | LKQFRHMDG |
| 15748 | LLYMAHCPP |
| 15749 | LMDGCTPFM |
| 15750 | LMEVYRSKA |
| 15751 | LMHYGMVWP |
| 15752 | LMSKENNCN |
| 15753 | LNGECVVGY |
| 15754 | LNTDVAEAF |
| 15755 | LNVGKKGYG |
| 15756 | LQQCEPMKS |
| 15757 | LQSEHNEDE |
| 15758 | LSICDQYDV |
| 15759 | LSMRCMDQW |
| 15760 | LSNFSWQYG |
| 15761 | LSVKHVHYS |
| 15762 | LTCMWGVYP |
| 15763 | LTTLVEAIP |
| 15764 | LTTQWNAKG |
| 15765 | LTVFSNGGV |
| 15766 | LTWVFGPFS |
| 15767 | LVDTWNNHR |
| 15768 | LWCKTWHNY |
| 15769 | LWDGEAWCW |
| 15770 | LWNMIKPLY |
| 15771 | LYELQQFGA |
| 15772 | LYHWWGYIE |
| 15773 | LYQSTFDWR |
| 15774 | MAADRWPLG |
| 15775 | MAGQSAAYW |
| 15776 | MAHIEGVAN |
| 15777 | MASDTDYSF |
| 15778 | MAVMIANAA |
| 15779 | MCGLTKFRP |
| 15780 | MCPPYQQAE |
| 15781 | MCSSQHSYS |
| 15782 | MDDDNDPVF |
| 15783 | MDKDSEIMG |
| 15784 | MEEYNTTTC |
| 15785 | MEGGHPYRT |
| 15786 | MESKYWHSD |
| 15787 | MFHDTLGMF |
| 15788 | MFMRLGTLP |
| 15789 | MFNMYEGSQ |
| 15790 | MFNWAEMED |
| 15791 | MFTDNTFKC |
| 15792 | MGIQTNYEA |
| 15793 | MGNMPASMA |
| 15794 | MGRTFFEEF |
| 15795 | MGRWENIWC |
| 15796 | MHDMLMGQM |
| 15797 | MHFQAWSGA |
| 15798 | MHIGVFTQA |
| 15799 | MIAFWSTWR |
| 15800 | MIAWWGSPG |
| 15801 | MIKPLTCNK |
| 15802 | MIKQTGAEW |
| 15803 | MINNSKTPY |
| 15804 | MIRTQCCWI |
| 15805 | MISQMMSNA |
| 15806 | MIYWTVDQQ |
| 15807 | MKASKDGGK |
| 15808 | MKEEWGHIP |
| 15809 | MLSWVYPCM |
| 15810 | MMAMKTDSH |
| 15811 | MMCQIMHSC |
| 15812 | MMNGMMGGN |
| 15813 | MMQEIGAEA |
| 15814 | MMRGPESDR |
| 15815 | MMVQQMFFL |
| 15816 | MNESTHIHP |
| 15817 | MPCCWWVVT |
| 15818 | MPCPLPKYR |
| 15819 | MPGYQIHNA |
| 15820 | MPKDWVPDA |
| 15821 | MPLTHSMHN |
| 15822 | MPNAFYQAC |
| 15823 | MPSQNGTKH |
| 15824 | MQLGEHSWY |
| 15825 | MQLLGDSEY |
| 15826 | MQSQEDHSP |
| 15827 | MRVWRYCDR |
| 15828 | MSSCKKMDA |
| 15829 | MTGEKTGNA |
| 15830 | MTLSWWGIG |
| 15831 | MTMLKMAGR |
| 15832 | MTNGNTVGA |
| 15833 | MTNIMQQGK |
| 15834 | MVDGDLPMK |
| 15835 | MVDKGFAGQ |
| 15836 | MVESELMMC |
| 15837 | MVGCCCHFG |
| 15838 | MVGPAQIEL |
| 15839 | MVHFTSHPI |
| 15840 | MVLTDAHTA |
| 15841 | MVQCMGQLA |
| 15842 | MVQPGHFES |
| 15843 | MVVASSASD |
| 15844 | MWDSVYYPF |
| 15845 | MYAPRCQTE |
| 15846 | MYGYTMNSG |
| 15847 | MYSCWTVNT |
| 15848 | MYSNLVHWS |
| 15849 | MYSYPHRRS |
| 15850 | MYTTHCHGM |
| 15851 | NAGLVNLGK |
| 15852 | NAQEEPESW |
| 15853 | NARYDKDNL |
| 15854 | NAVEQYVHH |
| 15855 | NCKEKEDCQ |
| 15856 | NCMASGWRG |
| 15857 | NCWINTRLP |
| 15858 | NDAKNLEGW |
| 15859 | NEKEYSWAA |
| 15860 | NEKHMSYCF |
| 15861 | NERHTFCVD |
| 15862 | NEWLMYLLT |
| 15863 | NFALATTTE |
| 15864 | NFTHREKEC |
| 15865 | NGKCCHPGP |
| 15866 | NHALMHGFQ |
| 15867 | NHCINSTEL |
| 15868 | NHESKDASW |
| 15869 | NIGFQMTMG |
| 15870 | NIGYANLWC |
| 15871 | NIQFQVGNY |
| 15872 | NIQWITDVG |
| 15873 | NIVHYCNPS |
| 15874 | NKSDEQLVQ |
| 15875 | NLTFCLFMG |
| 15876 | NMGVYQFLG |
| 15877 | NMVEYKQTA |
| 15878 | NNDCENPPL |
| 15879 | NNEFDVQHS |
| 15880 | NNMEIMKTC |
| 15881 | NPKQTHWDY |
| 15882 | NPRGEFMAH |
| 15883 | NQLQPICKP |
| 15884 | NSAHELHSP |
| 15885 | NSGTCPAPH |
| 15886 | NSSTVCNNR |
| 15887 | NSVMQMCVD |
| 15888 | NTFMPNGAN |
| 15889 | NTHMAHKSE |
| 15890 | NTQTLMTNG |
| 15891 | NTSLVRESE |
| 15892 | NVECVQQQW |
| 15893 | NVHDDHGAR |
| 15894 | NVMAGHDHE |
| 15895 | NVMFEQDDR |
| 15896 | NVQMQTLSW |
| 15897 | NVWTCDVCS |
| 15898 | NWFKMQAGM |
| 15899 | NYAETPCYS |
| 15900 | NYNFYATHV |
| 15901 | NYQNNMTFN |
| 15902 | PDVLHPNND |
| 15903 | PFRTDVRGS |
| 15904 | PGGTPRPEY |
| 15905 | PGKTWLWEL |
| 15906 | PGLDSTMHY |
| 15907 | PHPKSHKQH |
| 15908 | PIKYKHRQP |
| 15909 | PIRREWHLW |
| 15910 | PKEQYEWKD |
| 15911 | PKVDKRHKL |
| 15912 | PLASMWQHD |
| 15913 | PLMLLSKAM |
| 15914 | PLTFRGHNH |
| 15915 | PMEKFYCGC |
| 15916 | PNTERTYEC |
| 15917 | PQHAGRHHE |
| 15918 | PTDFIWPMM |
| 15919 | PTDQDQMLE |
| 15920 | PTKMHNHPW |
| 15921 | PVKLETHES |
| 15922 | PWGRSLVHC |
| 15923 | PWPSMACQK |
| 15924 | PYAPKNCCV |
| 15925 | QAIGRDQSF |
| 15926 | QAMWGNGQG |
| 15927 | QASVHVMAD |
| 15928 | QDHALVYRN |
| 15929 | QDYYSKPEW |
| 15930 | QEGIRTDVS |
| 15931 | QEMPLPMKS |
| 15932 | QFAEWWTMA |
| 15933 | QFILSHRRD |
| 15934 | QFLPCNFRC |
| 15935 | QFMTCQNYP |
| 15936 | QFVSWYTYR |
| 15937 | QGEMPDMFK |
| 15938 | QGNSPYHEA |
| 15939 | QGQPEVVVW |
| 15940 | QGQPTDASG |
| 15941 | QGYHFAWND |
| 15942 | QHALNQAVA |
| 15943 | QHIHEIGKK |
| 15944 | QIAMATMFR |
| 15945 | QIDNVRHHP |
| 15946 | QIMANACQN |
| 15947 | QITDMQDMQ |
| 15948 | QLVEAQLPP |
| 15949 | QLVEKEDMN |
| 15950 | QMSPAGCTE |
| 15951 | QNKVTQREC |
| 15952 | QPSPGHAYP |
| 15953 | QQDAAFHWT |
| 15954 | QQGFIVYWH |
| 15955 | QQIHMGHQA |
| 15956 | QQTLFRTGS |
| 15957 | QQYRASNFA |
| 15958 | QSCVTLQTN |
| 15959 | QSEFLVKHI |
| 15960 | QSMDCNWDG |
| 15961 | QSPSDIQCT |
| 15962 | QTADHRKGM |
| 15963 | QTATMAVWN |
| 15964 | QTDHFDSCR |
| 15965 | QTRAIVLLD |
| 15966 | QTYAQTHRT |
| 15967 | QVDWLKSSF |
| 15968 | QVEDELANY |
| 15969 | QVEMKDCDQ |
| 15970 | QVFPCDYEN |
| 15971 | QVMRCDFDD |
| 15972 | QVNLENHSC |
| 15973 | QVSVDCSQC |
| 15974 | QVVVIVWQA |
| 15975 | QVWINNVNI |
| 15976 | QWGTMMKNQ |
| 15977 | QWMTGMCWE |
| 15978 | QYAHISAHS |
| 15979 | QYQSVSNFS |
| 15980 | RCAQYEVAG |
| 15981 | RCLKRKTCF |
| 15982 | RCTGFDDFE |
| 15983 | RDETSFRHC |
| 15984 | RDSDKERCK |
| 15985 | RFHTDYRRH |
| 15986 | RFVMRPKES |
| 15987 | RGEQGVLTL |
| 15988 | RGLFDNWIF |
| 15989 | RGTYSYSAR |
| 15990 | RHLDQIPWP |
| 15991 | RHSWHPHFE |
| 15992 | RHYFYQDDD |
| 15993 | RIHEGSISG |
| 15994 | RKKGELCSR |
| 15995 | RKWCEFDAI |
| 15996 | RMCCSDSGE |
| 15997 | RMEQQGVMN |
| 15998 | RMESHIAQT |
| 15999 | RMHMPWCHG |
| 16000 | RNPHMDQSM |
| 16001 | RPIHDFGVY |
| 16002 | RTDQMELAT |
| 16003 | RTETKSTYF |
| 16004 | RTYWLDDGK |
| 16005 | RVDQEGGRD |
| 16006 | RYECYRSPF |
| 16007 | SAESRQGES |
| 16008 | SATPVGKEY |
| 16009 | SAVHRNDVA |
| 16010 | SAVHYTYMD |
| 16011 | SCAFVDEWS |
| 16012 | SCDAVDGLW |
| 16013 | SCDLKHDSG |
| 16014 | SCTDFNSMG |
| 16015 | SDHYNPEPY |
| 16016 | SEEESKEGG |
| 16017 | SEFCAWKCD |
| 16018 | SELVRWKHS |
| 16019 | SEQNRGSHT |
| 16020 | SERKDQRMW |
| 16021 | SEWQIFFFN |
| 16022 | SFRIANQTN |
| 16023 | SFYSTGIWG |
| 16024 | SGPDMTAGN |
| 16025 | SHRWVMKEH |
| 16026 | SHSHQPSAS |
| 16027 | SICGAVQNF |
| 16028 | SIKGRDWGD |
| 16029 | SIMVSTHNN |
| 16030 | SIQPLAATT |
| 16031 | SKMEEALRM |
| 16032 | SLRLELRPQ |
| 16033 | SMKHRMQAF |
| 16034 | SMLQFLSDD |
| 16035 | SMYFKPTQD |
| 16036 | SNEMCGFAM |
| 16037 | SNLQIQDAK |
| 16038 | SNSPTCYAY |
| 16039 | SNTMRLNAE |
| 16040 | SNYAPFYCD |
| 16041 | SPHCYESTP |
| 16042 | SPMVKKSGT |
| 16043 | SPMYEMSAF |
| 16044 | SPNHDAGQN |
| 16045 | SQDTEHCWA |
| 16046 | SQGCQVKQC |
| 16047 | SQMPHSKAA |
| 16048 | SQSNFIIME |
| 16049 | SRHWRPQSC |
| 16050 | SSGLYWKKN |
| 16051 | SSLNYSPGA |
| 16052 | STAAYQFEE |
| 16053 | STAEDQADH |
| 16054 | STAPDNTAF |
| 16055 | STDALWQEN |
| 16056 | STDCIFFQH |
| 16057 | STDSMDAEA |
| 16058 | STEHLGRND |
| 16059 | STEPSGIHQ |
| 16060 | STGQDQEHT |
| 16061 | STHGQASAI |
| 16062 | STKQVEALG |
| 16063 | STVNTSSPE |
| 16064 | SVCLDIPCI |
| 16065 | SVDYTNALT |
| 16066 | SVENYERCQ |
| 16067 | SVRFDGNDW |
| 16068 | SVTVNNVHC |
| 16069 | SWAVLHQAQ |
| 16070 | SWDMEWDGP |
| 16071 | SWDVPLYNP |
| 16072 | SYCNLQFMR |
| 16073 | SYEKSVNMG |
| 16074 | SYQAVFGSI |
| 16075 | SYVQRHCVF |
| 16076 | TAFSGWKEQ |
| 16077 | TAGLGIESV |
| 16078 | TAMDCMCGT |
| 16079 | TAMKFPGGI |
| 16080 | TASPAFDES |
| 16081 | TASSWLMHQ |
| 16082 | TATGMNRCE |
| 16083 | TATSWWAFK |
| 16084 | TAVWMDKTS |
| 16085 | TCFMGWRCL |
| 16086 | TCRMGMEIG |
| 16087 | TDQTCSKCI |
| 16088 | TEEICQLRE |
| 16089 | TEKDNEEHQ |
| 16090 | TEKMSHCSE |
| 16091 | TESQSGGQP |
| 16092 | TFARRADYA |
| 16093 | TFPVGYNYT |
| 16094 | TGEWQQGKP |
| 16095 | THMTIDDAS |
| 16096 | TIGVDPSCQ |
| 16097 | TIKHILNHK |
| 16098 | TIKNYCQGR |
| 16099 | TIKYDLHFA |
| 16100 | TIMPRHRHA |
| 16101 | TITTFWDFE |
| 16102 | TKRPFKGQW |
| 16103 | TLHHNSDAK |
| 16104 | TLIYNAQVT |
| 16105 | TMDQKNDMD |
| 16106 | TMGTAAKSH |
| 16107 | TMPVDVMIP |
| 16108 | TMPWQGNHC |
| 16109 | TMQNTEPGI |
| 16110 | TMREFGEGD |
| 16111 | TNADSWDQE |
| 16112 | TNGVPPWWS |
| 16113 | TNHINFGWI |
| 16114 | TNHSFWIQG |
| 16115 | TPDCYSMGA |
| 16116 | TPNCGINHE |
| 16117 | TQCNTDQRT |

TABLE 77
Sequences of the 581 to 589 Region in
AAV5 VP1 Capsid Polypeptide that Drive
Lung Tissue Tropism

| | 581-589 |
|---|---|
| SEQ ID NO | Sequence |
| 18118 | QTNTMQMLS |
| 18119 | GPRHIEEDG |
| 18120 | RKADVEYQG |
| 18121 | SCDMQQAHQ |
| 18122 | DILMFEWFD |
| 18123 | DIRMFAWFD |
| 18124 | DIRMFELFD |
| 18125 | DIRMFEWFD |
| 18126 | DIRMFEWFG |
| 18127 | DIRMFEWFN |
| 18128 | DIRMFEWFY |
| 18129 | DIRMFEWVD |
| 18130 | DIRMFKWFD |
| 18131 | DIRMFVWFD |
| 18132 | DIRMIEWFD |
| 18133 | DIRMVEWFD |
| 18134 | DIRVFEWFD |
| 18135 | DMRMFEWFD |
| 18136 | EGKQAYFFP |
| 18137 | ELKQAYFFP |
| 18138 | EPKRLLRTV |
| 18139 | EPKRLLWTV |
| 18140 | EWEQAYFFP |
| 18141 | EWKPAYFFP |
| 18142 | EWKQACFFP |
| 18143 | EWKQASFFP |
| 18144 | EWKQAYFFA |
| 18145 | EWKQAYFFP |
| 18146 | EWKRAYFFP |
| 18147 | EWRQAYFFP |
| 18148 | GIRMFEWFD |
| 18149 | GMAHRFRGR |
| 18150 | GMAHWFRGR |
| 18151 | GMTHRFRGR |
| 18152 | HIESFTYCN |
| 18153 | HTESFAYCN |
| 18154 | HTESFTYCN |
| 18155 | HTESFTYCS |
| 18156 | HTESFTYCT |
| 18157 | HTKSFTYCN |
| 18158 | HTQFINGRG |
| 18159 | HTQFTDGRG |
| 18160 | HTQFTNGRG |
| 18161 | MSHSRIQEM |
| 18162 | NIRMFEWFD |
| 18163 | NISFPNQSE |
| 18164 | NTESFTYCN |
| 18165 | QLDARWYRC |
| 18166 | QLNARWYRC |
| 18167 | QLNARWYRW |
| 18168 | QLNAWWYRC |
| 18169 | QLSARWYRC |
| 18170 | RLDYRHYSK |
| 18171 | RLNYRHCSK |
| 18172 | RLNYRHYSK |
| 18173 | RLNYRHYSR |
| 18174 | RLNYRHYST |
| 18175 | RLNYRPYSK |
| 18176 | RLNYWHYSK |
| 18177 | SKNLCTMAG |
| 18178 | SKNLYTMAC |
| 18179 | SKNLYTMAD |
| 18180 | SKNLYTMAG |
| 18181 | SKNLYTMAV |
| 18182 | SKNLYTMSG |
| 18183 | SKNLYTMVG |
| 18184 | SKNLYTVAG |
| 18185 | TMTSTLQST |
| 18186 | VWKQAYFFP |
| 18187 | GTKLIWPYQ |
| 18188 | CSTLAKHDE |
| 18189 | DVIHTTHSG |
| 18190 | DFMWYHICS |
| 18191 | DVEFCMKQY |
| 18192 | MMLGYMEQD |
| 18193 | DFNGSNWLP |
| 18194 | AGRFKFPQP |
| 18195 | AMYQCPSMD |
| 18196 | ANWKHWQWR |
| 18197 | ATVNSDTLT |
| 18198 | AVGHFEYVL |
| 18199 | AVWTMLEHC |
| 18200 | CQVWWENRD |
| 18201 | CQYAVTKMG |
| 18202 | CSGVDCTGA |
| 18203 | DCSSCMKDL |
| 18204 | DRKLGDAHG |
| 18205 | DTARWDVMT |
| 18206 | DVVNCTVHT |
| 18207 | EICSRGRTI |
| 18208 | ELGHCLLYE |
| 18209 | ELQSFMQYG |
| 18210 | ERFKTFCEE |
| 18211 | FASSHWYQG |
| 18212 | FYLWDTRET |
| 18213 | GEANMAPSR |
| 18214 | GMSEQIAIT |
| 18215 | GWSTDWTET |
| 18216 | IASKFNICC |
| 18217 | IFVYCEKMP |
| 18218 | ITSSPNHIF |
| 18219 | IVDTSARYQ |
| 18220 | KANWVESME |
| 18221 | KTGWGGITG |
| 18222 | KWKVITTCY |
| 18223 | LLCTCGWCE |
| 18224 | LSYENDAFQ |
| 18225 | MCSHNLPKS |
| 18226 | MHTMTNAMK |
| 18227 | MNTHEQWGG |
| 18228 | MSLMPHGWH |
| 18229 | MTDLMYIQP |
| 18230 | MVECLYYHG |
| 18231 | MVFLQCQQN |
| 18232 | MWNTSNLFH |
| 18233 | NVNLQMMMT |
| 18234 | NYNGYSDAQ |
| 18235 | PSTKPPKCK |
| 18236 | QDWSDFNDC |
| 18237 | QNVQLENSQ |
| 18238 | SKTKKRFAW |
| 18239 | SRYDTNCWW |
| 18240 | STHNRLDWQ |
| 18241 | TGDEIVGTY |
| 18242 | TSIPPRHNP |
| 18243 | VAAYNFPTC |
| 18244 | VMEGVRNME |
| 18245 | WTHMYENID |
| 18246 | WYNQGAEYH |
| 18247 | WYWRLDCLW |
| 18248 | YATIPGVFP |
| 18249 | YCHRSDCDR |
| 18250 | YMDEMITEL |
| 18251 | AAMPTHHTM |
| 18252 | AANIMISQA |
| 18253 | AANVTQDTS |
| 18254 | AAPKYSAHQ |
| 18255 | AAQPHFVVA |
| 18256 | AASDWCRYS |
| 18257 | AASWQEFFP |
| 18258 | AAYFVGENC |
| 18259 | ACEWKQHWA |
| 18260 | ACGKMHGPN |
| 18261 | ACGVVLKAA |
| 18262 | ACKQFKKHA |
| 18263 | ACLQQDIAM |
| 18264 | ADAHQRTVS |
| 18265 | ADGQMLQAH |
| 18266 | ADRQSQMHL |
| 18267 | AFLELAMYP |
| 18268 | AFTEGQLNE |
| 18269 | AGNRWWWSI |
| 18270 | AHASWKCWF |
| 18271 | AIGTLAPNL |
| 18272 | AILMQQGAG |
| 18273 | AIMHPPYCE |
| 18274 | AIQSGNWSS |
| 18275 | AKLEQMDSA |
| 18276 | AKTHCHCWW |
| 18277 | ALHHCIKKA |
| 18278 | ALIQFMMDQ |
| 18279 | AMAYSWTMA |
| 18280 | AMCFNKCWQ |
| 18281 | AMSQQHDHQ |
| 18282 | AMSTLGECP |
| 18283 | APQFYVSYD |
| 18284 | APTKVHCQG |
| 18285 | AQNPFDIDA |
| 18286 | AQNREGAKL |
| 18287 | AQTFCSFDR |
| 18288 | ARSGINGHE |
| 18289 | ASGCLQQIS |
| 18290 | ASIDVASWG |
| 18291 | ASTPWCWFE |
| 18292 | ATDNSDTLT |
| 18293 | ATGIDPLGA |
| 18294 | ATMIKFECT |
| 18295 | ATYISAHDA |
| 18296 | AVEHIERSF |
| 18297 | AVISPERYH |
| 18298 | AVNDWNNDW |
| 18299 | AVTRPSNKM |
| 18300 | AWDPMTSTF |
| 18301 | AWGGFNAEM |
| 18302 | AWILFWWFV |
| 18303 | AYCSEERSP |
| 18304 | AYENVQHPN |
| 18305 | AYEPFASSF |
| 18306 | AYNRMMLTC |
| 18307 | CAFPVNITA |
| 18308 | CAGVTSHGE |
| 18309 | CAICNAQCP |
| 18310 | CAQSVHFHK |
| 18311 | CAYEVYDGK |
| 18312 | CFCKNGTYL |
| 18313 | CGEWLNECS |
| 18314 | CHHFNMPSQ |
| 18315 | CILHVTDNG |
| 18316 | CKSNWHIFK |
| 18317 | CLMSNITHI |
| 18318 | CPFQHLGPK |
| 18319 | CQQYWNAAW |
| 18320 | CSAVYLANH |
| 18321 | CVDEYYNSS |
| 18322 | CVIDWKAMS |
| 18323 | CVTKTMWTG |
| 18324 | CWRSGSMNL |
| 18325 | CWSITNQDH |
| 18326 | CYASMSASY |
| 18327 | CYDWYEGAE |
| 18328 | CYSENDHQV |
| 18329 | DAAHMGYEP |
| 18330 | DAGHSFQIF |
| 18331 | DAGPIWYSS |
| 18332 | DANQRATNS |
| 18333 | DANSLNSWP |
| 18334 | DARSMAPVE |
| 18335 | DAYSVHHYV |
| 18336 | DCNSLINVA |
| 18337 | DCRHTILYV |
| 18338 | DDMLMQFQH |
| 18339 | DFGCMDYGP |
| 18340 | DFHSYLKQQ |
| 18341 | DGFQQGEGI |
| 18342 | DINRSGNQC |
| 18343 | DKWCCEMTA |
| 18344 | DLEYIWHWG |
| 18345 | DLIGSGPWF |
| 18346 | DMHECIGCS |
| 18347 | DMHRQHLIE |
| 18348 | DMLKFHFNY |
| 18349 | DMMDNQIAS |
| 18350 | DNIRYPCHS |
| 18351 | DNQFFNGDY |
| 18352 | DPVWLPFMF |
| 18353 | DQFARHKKY |
| 18354 | DRPKKNLCC |
| 18355 | DRPSTGWYL |
| 18356 | DSDIVNDYC |
| 18357 | DTKVKKEFG |
| 18358 | DTMLRLCYP |
| 18359 | DTNWMQWNH |
| 18360 | DTSLNDGWM |
| 18361 | DVCCQFSAY |
| 18362 | DVLVATQVQ |
| 18363 | DYQRKTRYS |
| 18364 | EACKCSVVP |
| 18365 | EAFYLQKPG |
| 18366 | EAIQDAHQM |
| 18367 | EANKGASQD |
| 18368 | ECCNMSPPG |
| 18369 | ECGRFAPME |
| 18370 | ECHKCEVCV |
| 18371 | EDRPMAMME |
| 18372 | EEDKYNVVQ |
| 18373 | EEKKLTVIM |
| 18374 | EFGTYDNIG |
| 18375 | EFRYGPQYG |
| 18376 | EFVIAEETS |
| 18377 | EGANYHWDH |
| 18378 | EGAQQESVG |
| 18379 | EGARYTKFH |
| 18380 | EGGMGPQQS |
| 18381 | EGKWMSVHH |
| 18382 | EGRPQYTDP |
| 18383 | EGSFVLSTE |
| 18384 | EHAPMVHAL |
| 18385 | EHQRMKFRN |
| 18386 | EHVQKVNYP |
| 18387 | EHYCVTAWY |
| 18388 | EIDLWRTPT |
| 18389 | EIHDEASCY |
| 18390 | EIHKEMGFG |
| 18391 | EIMFYMRKQ |
| 18392 | EINIKKKQM |
| 18393 | EINKMAALG |
| 18394 | EIRTVESAG |
| 18395 | EISWIPREN |
| 18396 | EKHSVENYG |
| 18397 | EKIDWQSCV |
| 18398 | EMAICAEHS |
| 18399 | EMGRLPNHN |
| 18400 | EMVDQIVDF |
| 18401 | EMYPEAAAK |
| 18402 | ENYPPSQCA |
| 18403 | EPNHPESLA |
| 18404 | EQKVGNQLS |
| 18405 | ERCMSEWSF |
| 18406 | ERWSGTRRG |
| 18407 | ERYHFMTHQ |
| 18408 | ESKYNRKYN |
| 18409 | ESRDAVCPD |
| 18410 | ETCHTYAQH |
| 18411 | ETHGDRKSR |
| 18412 | ETICCDSAH |
| 18413 | ETMGVHYCE |
| 18414 | ETRMQRRSQ |
| 18415 | ETWPLIDDA |
| 18416 | EVADCLKHF |
| 18417 | EVCNEMPEP |
| 18418 | EVELDERLS |
| 18419 | EVELYGCIC |
| 18420 | EVESQEHLH |
| 18421 | EVTRETDYM |
| 18422 | EYCMLEDYQ |
| 18423 | EYNRRYLLE |
| 18424 | EYQNYQLVT |
| 18425 | EYTRTQWAH |
| 18426 | FHHLNTPEM |
| 18427 | FIFEEQRWA |
| 18428 | FKETWLFPT |
| 18429 | FLCCPKECP |
| 18430 | FQRLEMKQH |
| 18431 | FQRWEMKQH |
| 18432 | FSGKYDQRG |
| 18433 | FSQYPSAFA |
| 18434 | FSRPFEQYH |
| 18435 | FVAQMWDDH |
| 18436 | FYRQLDEAG |
| 18437 | GASSKSLDA |
| 18438 | GDIYYKCWV |
| 18439 | GDLWNNHLE |
| 18440 | GFEDRGNQE |
| 18441 | GFMLRMSTD |
| 18442 | GFSLFMMNR |
| 18443 | GGMTVLIEY |
| 18444 | GHQLMFFKF |
| 18445 | GIKEQRRFV |
| 18446 | GIRMDLCTF |
| 18447 | GITHDFMAG |
| 18448 | GIVIKPHGE |
| 18449 | GIYPQFTWQ |
| 18450 | GKEFALSAF |
| 18451 | GLATLGLSG |
| 18452 | GLGMVHRMH |
| 18453 | GMMWRSKWI |
| 18454 | GMRCWRHRL |
| 18455 | GMRYSEWHW |
| 18456 | GMVDVCDCC |
| 18457 | GQHTIHKNS |
| 18458 | GQTVQPCST |
| 18459 | GSCCLNNET |
| 18460 | GSLTMPDAH |
| 18461 | GSYYVNWDQ |
| 18462 | GTCLQDNYN |
| 18463 | GTGWEASAD |
| 18464 | GTGWGASAD |
| 18465 | GTLSSSIVE |
| 18466 | GTNQVCLFT |
| 18467 | GVDWRHQQN |
| 18468 | GVISEGISA |
| 18469 | GVMVIAGMG |
| 18470 | GVNQYDEMI |
| 18471 | GVVQMVIGD |
| 18472 | GWMDLMSAR |
| 18473 | GYDHHGTEH |
| 18474 | HCMEYIHHA |
| 18475 | HDCEFLKDE |
| 18476 | HEVERRRAK |
| 18477 | HGMQLEAFF |
| 18478 | HHNIKEAWM |
| 18479 | HIESYMCRD |
| 18480 | HIRHRDENG |
| 18481 | HKNRLLRSV |
| 18482 | HKSRATHTG |
| 18483 | HNHILIQTS |
| 18484 | HPMDGLIQV |
| 18485 | HSEHQGRMV |
| 18486 | HSFWIAQMQ |
| 18487 | HSGIGLTMQ |
| 18488 | HSWLENSAC |
| 18489 | HVEYVQKYE |
| 18490 | HVFQVSDED |
| 18491 | HWHNVISKD |
| 18492 | IADTPDALQ |
| 18493 | IEDISKKWA |
| 18494 | IFQPCFRDW |
| 18495 | IGQSTQYCA |
| 18496 | IHQSVIQKG |
| 18497 | IISRYDNDD |
| 18498 | IKYSIDHEN |
| 18499 | IMELTHHNA |
| 18500 | INAVPWQLL |
| 18501 | IPAGMTPWC |
| 18502 | IQAPTENNN |
| 18503 | IRPMYETEE |
| 18504 | ISAPGCVFC |
| 18505 | ISDRMNYMN |
| 18506 | ISERVHQTE |
| 18507 | ISQFGANVH |
| 18508 | ISSDKKIMG |
| 18509 | ITAPRMVET |
| 18510 | ITGCIQKAK |
| 18511 | ITKWSLGNP |
| 18512 | IVRTMDNEQ |
| 18513 | IYATNIAWP |
| 18514 | IYMHKIALF |
| 18515 | IYSQIGRFD |
| 18516 | KCAGWMPSS |
| 18517 | KDVEECNSL |
| 18518 | KEYPRIGRM |
| 18519 | KGSNDGADK |
| 18520 | KHQSTCGQD |
| 18521 | KMDFWAVPI |
| 18522 | KQSELGIGC |
| 18523 | KSSNYHSWP |
| 18524 | KTEQGGDAE |
| 18525 | KTTDECWHV |
| 18526 | KVEDVGDYA |
| 18527 | KVTEAAKTM |
| 18528 | KWCMFFDQH |
| 18529 | KYELSQCFG |
| 18530 | KYHQAGQLC |
| 18531 | LADSSMLWE |
| 18532 | LADVQFLYV |
| 18533 | LADWSYEAS |
| 18534 | LATDVPVSE |
| 18535 | LCFQSVHEE |
| 18536 | LCTAVFRDQ |
| 18537 | LFMHTMWGA |
| 18538 | LFNCTVWLE |
| 18539 | LFRYQRKTQ |
| 18540 | LGDWAPASI |
| 18541 | LGNSFKRAR |
| 18542 | LGQQAMPLK |
| 18543 | LHFKIEQVD |
| 18544 | LHNNMNAFG |
| 18545 | LIALDMCHP |
| 18546 | LIQSYVNTG |
| 18547 | LIRAKHRTM |
| 18548 | LKKEVQRYQ |
| 18549 | LKSEVSIME |
| 18550 | LMYWKNGMS |
| 18551 | LNAWVNRHS |
| 18552 | LNHKYERMT |
| 18553 | LNKYNPHKT |
| 18554 | LQDLTQLCK |
| 18555 | LRQCPLNKL |
| 18556 | LSDQCLDLA |
| 18557 | LSNQQWYAG |
| 18558 | LSRSGHDQE |
| 18559 | LSTDNNIQF |
| 18560 | LSTMEWKTN |
| 18561 | LSTNPQHCG |
| 18562 | LTAMKKQNA |
| 18563 | LTEVKWCNC |
| 18564 | LTGYLNDMT |
| 18565 | LVDKNMFIN |
| 18566 | LVTQWVPAY |
| 18567 | LWAWSGEPK |
| 18568 | LWNDMHFHG |
| 18569 | MAGRDDGRA |
| 18570 | MAIIRLAEM |
| 18571 | MAKSSRNDQ |
| 18572 | MAPKFSSYG |
| 18573 | MASFPMENK |
| 18574 | MCQHEQATF |
| 18575 | MCTSVTMNG |
| 18576 | MEHSTHDDD |
| 18577 | MENYQWLAP |
| 18578 | MFGCIIGDF |
| 18579 | MGSKRKPWT |
| 18580 | MGVKVDGTN |
| 18581 | MHDGYRGMQ |
| 18582 | MHHCYQTFA |
| 18583 | MHIAQMLGP |
| 18584 | MHSNLHWYG |
| 18585 | MHSPCSGPW |
| 18586 | MIDVLSWLE |
| 18587 | MIESQGDLQ |
| 18588 | MILPSMAWC |
| 18589 | MISCGGDDH |
| 18590 | MKDHWDFYG |
| 18591 | MMIGTYMGE |
| 18592 | MMNQTCCHM |
| 18593 | MMVVRGKVE |
| 18594 | MNSCNNYCM |
| 18595 | MPVERNVTP |
| 18596 | MQEDCMSGE |
| 18597 | MQNEDVPHT |
| 18598 | MQQECVDMW |
| 18599 | MQQGYGIFA |
| 18600 | MRFILLADW |
| 18601 | MSQCIVMHR |
| 18602 | MTMQDINGQ |
| 18603 | MVWNTSMDP |
| 18604 | MYTMQQSGI |
| 18605 | NAAPISHIF |
| 18606 | NCENEWRLN |
| 18607 | NCERRKSAG |
| 18608 | NCGSLPVTE |
| 18609 | NERPYEGMG |
| 18610 | NFFSLHMMS |
| 18611 | NGCECHECW |
| 18612 | NGFQTRMRV |
| 18613 | NGWMNQSAG |
| 18614 | NIQWQWMTG |
| 18615 | NLNVMFSME |
| 18616 | NQEGQGMFT |
| 18617 | NSAHIMKDG |
| 18618 | NSMHGDCQW |
| 18619 | NTCPVESAH |
| 18620 | NTDNNWTSG |
| 18621 | NTNLLEITH |
| 18622 | NTTYRTEYF |
| 18623 | NVVFKQSAH |
| 18624 | NVWVIKDIC |
| 18625 | NVYFSWMWT |
| 18626 | NWTCLMSIP |
| 18627 | NYPLQWGVE |
| 18628 | PATDQIDEC |
| 18629 | PATRVFADI |
| 18630 | PDWMDWNHY |
| 18631 | PFVKSNIAL |
| 18632 | PKQLFWYWW |
| 18633 | PLFAITTIE |
| 18634 | PLTMGHGEC |
| 18635 | PQHNCGYNN |
| 18636 | PSENNKRKK |
| 18637 | PVDWIIAAE |
| 18638 | QAIFVRQLN |
| 18639 | QCGHDHQYT |
| 18640 | QCKYSERGT |
| 18641 | QFAEKPGSW |
| 18642 | QFEGGDSMG |
| 18643 | QGTGRYAWG |
| 18644 | QGYRKLMNQ |
| 18645 | QHHTYMCIS |
| 18646 | QIMFNHQNL |
| 18647 | QIVGNQKRE |
| 18648 | QKMEMFEGS |
| 18649 | QLETFTVHN |
| 18650 | QNDVMMGNH |
| 18651 | QQSFFFGEQ |
| 18652 | QQSSMATEY |
| 18653 | QQWDVAMMC |
| 18654 | QRVDQQSLG |
| 18655 | QSEWWRRHA |
| 18656 | QSHSMMYFG |
| 18657 | QSHSTDYCS |
| 18658 | QSQPDNKGM |
| 18659 | QSQYKAECK |
| 18660 | QSSGEERGT |
| 18661 | QSSWDGQDD |
| 18662 | QSTQTSYIS |
| 18663 | QSTSDECNL |
| 18664 | QTDIWMMWA |
| 18665 | QTDSVMECT |
| 18666 | QTTHFRYFH |
| 18667 | QTTQKSEER |
| 18668 | QVDTRYMNE |
| 18669 | QVGMVAISQ |
| 18670 | QVLWANNHD |
| 18671 | QVSFEWYKC |
| 18672 | QWPLNPYGK |
| 18673 | QWRILDVVK |
| 18674 | QYAMSYAGE |
| 18675 | QYYEHEFLD |
| 18676 | RADHTIDFS |
| 18677 | RCDTSQWWQ |
| 18678 | REGWPGHKY |
| 18679 | RENLCNVPP |
| 18680 | RGGLIHFST |
| 18681 | RHLDIQNHD |
| 18682 | RHMESLNSA |
| 18683 | RIHLWENNP |
| 18684 | RLCWMTPCC |
| 18685 | RLENFHEAH |
| 18686 | RMELRMIFP |
| 18687 | RMEPLHCCE |
| 18688 | RPYPAMFKG |
| 18689 | RQHHVGNEV |
| 18690 | RQPPLKWKR |
| 18691 | RSNEKGMNG |
| 18692 | RTDFLACHC |
| 18693 | RTINFSLMI |
| 18694 | RTMYWPSVN |
| 18695 | RVDFTPIVH |
| 18696 | RVGMDPRDG |
| 18697 | RWAEMHFID |
| 18698 | RWAYLEEFR |
| 18699 | SAGWDGKVC |
| 18700 | SAVNYFMFG |
| 18701 | SAYVVCSHD |
| 18702 | SCDPRHVDE |
| 18703 | SEANMAPSR |
| 18704 | SELDQYTMA |
| 18705 | SFLTTAYEA |
| 18706 | SGHPRHMCY |
| 18707 | SHMPFSDMC |
| 18708 | SHMQMNWVD |
| 18709 | SHTECHTWG |
| 18710 | SIISDEYFT |
| 18711 | SITPRDEKP |
| 18712 | SKIKPADPN |
| 18713 | SLSVKFKTN |
| 18714 | SMQQAHGWK |
| 18715 | SPGSCQAFE |
| 18716 | SQNQYIKAA |
| 18717 | SREICQLEY |
| 18718 | SSPWKDFRQ |
| 18719 | SSQIKTSSF |
| 18720 | STACAESGW |
| 18721 | STHPYMLSK |
| 18722 | STHQPDCFR |
| 18723 | STMCMVRDS |
| 18724 | STTHLQYYC |
| 18725 | SVGPQLYLT |
| 18726 | SVGTWQNMG |
| 18727 | SVHKAENWW |
| 18728 | SVLMQMGSE |
| 18729 | SVTTCHMPL |
| 18730 | TADHNLFDY |
| 18731 | TADWEDQTP |
| 18732 | TATEGVWGC |
| 18733 | TCCYDQRGP |
| 18734 | TCDSNATWY |
| 18735 | TCEHRLMAT |
| 18736 | TCGTTANFE |
| 18737 | TFWYTGHWG |
| 18738 | TGEYTNSWD |
| 18739 | THGCGQIMP |
| 18740 | THLSCRMWG |
| 18741 | TIGIPYSHD |
| 18742 | TKKDLDRQW |
| 18743 | TLALDCKWP |
| 18744 | TLCIREIMM |
| 18745 | TLTMMQGCS |
| 18746 | TMFAVTVKN |
| 18747 | TMGFLWYEQ |
| 18748 | TNKMLGRAP |
| 18749 | TPGRNNQGM |
| 18750 | TPIGVQKLA |
| 18751 | TPSQQAEQC |
| 18752 | TQAPIWENL |
| 18753 | TQEHSHEVS |
| 18754 | TSDVRLTEE |
| 18755 | TSGHMRNMF |
| 18756 | TSIDETELF |
| 18757 | TSIWINAVK |
| 18758 | TSQQFITAY |
| 18759 | TSSWLQCYI |
| 18760 | TSTWPGVIA |
| 18761 | TTAISLIGE |
| 18762 | TTAYSPNDR |
| 18763 | TTNLEQMIP |
| 18764 | TTQDGQMYS |
| 18765 | TTQSMQSGS |
| 18766 | TVCMFWVWC |
| 18767 | TVDPFKRIW |
| 18768 | TVEEAVWAN |
| 18769 | TVMMPMRLD |
| 18770 | TVNICLQHQ |
| 18771 | TWNCTFTGG |
| 18772 | VAHQSPNMM |
| 18773 | VAQPCGQYF |
| 18774 | VCIDHRAYS |
| 18775 | VCQQWLTQN |
| 18776 | VDYPRKCDK |
| 18777 | VGEPLAMFM |
| 18778 | VGQWFFKAG |
| 18779 | VIDCCYSCG |
| 18780 | VIEQHIQQF |
| 18781 | VKWDKKTKQ |
| 18782 | VLFTIGKDS |
| 18783 | VLHRTSYKH |
| 18784 | VMEFDKPSA |
| 18785 | VMKGVDKFH |
| 18786 | VNRCCYPNY |
| 18787 | VPEHMDRDQ |
| 18788 | VQGLLIFVS |
| 18789 | VQHVRYWHS |
| 18790 | VSFPTNTMQ |
| 18791 | VSLPHSKHL |
| 18792 | VSNHWMYHL |
| 18793 | VTAWWDTAE |
| 18794 | VTEVYERST |
| 18795 | VTGFSFTPN |
| 18796 | VTGTMLKDA |
| 18797 | VTKMPDFAP |
| 18798 | VTQYNPKMG |
| 18799 | VTRQVIESQ |
| 18800 | VTWSTHQWL |
| 18801 | VVWDVDYYF |
| 18802 | VWDALQNPM |
| 18803 | VYVQEGNCS |
| 18804 | WAHTNMVYT |
| 18805 | WFCFEDSPS |
| 18806 | WFMCDYNGD |
| 18807 | WGWSPWKKI |
| 18808 | WKTKVENPE |
| 18809 | WWCWMAGPV |
| 18810 | WWTHKYFYL |
| 18811 | YAHEEDQNP |
| 18812 | YAWISEYEA |
| 18813 | YDVTKLGEQ |
| 18814 | YEAWKVNID |
| 18815 | YHCCWTSHP |
| 18816 | YHNIATPIP |
| 18817 | YPNTWCVVV |
| 18818 | YQGPAVLCA |
| 18819 | YRTHCFKGH |
| 18820 | YSCHFNQKT |
| 18821 | YWNGRLKWD |
| 18822 | ITTSNVEND |
| 18823 | CCFPHYFVG |
| 18824 | MFELHDHQF |
| 18825 | RLCDTHKCL |
| 18826 | NNRALMSVE |
| 18827 | DSCIKGLAC |
| 18828 | MREKLVHDC |
| 18829 | TTYHHRNTL |
| 18830 | TWNTWQQDF |
| 18831 | DSASPIMGM |
| 18832 | MMAMKQGFY |
| 18833 | KACMSHQGR |
| 18834 | TIGEMVNPS |
| 18835 | NWNHCHQWR |
| 18836 | SEDKDQGAQ |
| 18837 | CAVAGGEWM |
| 18838 | NTMLSWGSP |
| 18839 | CSIRGFSAF |
| 18840 | TIDWCEPHC |
| 18841 | SFSHTDLEA |
| 18842 | AIEHGAWKC |
| 18843 | TLTTDKTYS |
| 18844 | KDGRNPHCL |
| 18845 | WHCVAPNSF |
| 18846 | ACIPQMHSA |
| 18847 | NYAMRRDSY |
| 18848 | IYMSTGNTP |
| 18849 | GYRNYFIWP |
| 18850 | MFTPCQEGL |
| 18851 | GSTWGASWH |
| 18852 | GQFRSGAYR |
| 18853 | VYILEGGIH |
| 18854 | NTQVISRWT |
| 18855 | CYMNFIVDG |
| 18856 | NHKMVQDAL |
| 18857 | DVYMAVVGS |
| 18858 | GITMHPMHH |
| 18859 | QMTSSTNCA |
| 18860 | MHGIARMQQ |
| 18861 | RVWSLEWHL |
| 18862 | EVYCYQLQS |
| 18863 | QENDERIWY |
| 18864 | AMTRCKAGH |
| 18865 | KPLASIGQY |
| 18866 | GAANRIQSW |
| 18867 | GHMFETFWG |
| 18868 | EAQPSEING |
| 18869 | ECLEVKRTC |
| 18870 | FGDHLRVMY |
| 18871 | WTHCTNNQI |
| 18872 | NAYYDSHCE |
| 18873 | EKYQIHWDR |
| 18874 | QALVGNAAE |
| 18875 | QSSSSYLWG |
| 18876 | LESVKMDCN |
| 18877 | QRSVFHQRS |
| 18878 | NDVWQPHPN |
| 18879 | EDQWISNPF |
| 18880 | DHLSNDAEM |
| 18881 | LHKAYVTDS |
| 18882 | AVDHYSNGA |
| 18883 | DMKQKAEYT |
| 18884 | IMYLKRCCT |
| 18885 | DQRHGNVSP |
| 18886 | RVTIMFTGT |
| 18887 | TTYPHNTHG |
| 18888 | PPRFLQTTG |
| 18889 | YPTMTFPEE |
| 18890 | GCEEVGRCQ |
| 18891 | MAAITQNVA |
| 18892 | WPTMLSAHM |
| 18893 | KCLPYMEMF |
| 18894 | AHAWLQHWA |
| 18895 | PDLWYEKSS |
| 18896 | DTRRNLCCD |
| 18897 | LIEKQRWML |
| 18898 | ENQSTMSHT |
| 18899 | QWMHCQLSR |
| 18900 | ICHIRTRGN |
| 18901 | YISEYVMMV |
| 18902 | QATGMHRWQ |
| 18903 | MFMTNVNSF |
| 18904 | KTVETYAAH |
| 18905 | HIKVEHEEV |
| 18906 | KMAGIGCWY |
| 18907 | QCTAHNTCM |
| 18908 | RTRISQMFG |
| 18909 | MGYAKHDSH |
| 18910 | PIWQGYWPW |
| 18911 | RCGEIMGLD |
| 18912 | LPSHCNLGS |
| 18913 | PAHINDQYM |
| 18914 | DRVNLMWKD |
| 18915 | QCVDCPPYC |
| 18916 | AVHTADVCC |
| 18917 | NQREWHGLA |
| 18918 | HIHMSDRGA |
| 18919 | PCCWEQSTN |
| 18920 | AFLHGQMWQ |
| 18921 | PLTIEVNCT |
| 18922 | IAKIGNCVW |
| 18923 | YFPSIQCYE |
| 18924 | NMPSLRTRI |
| 18925 | CKNHYMMAA |
| 18926 | SECEVLCSL |
| 18927 | ERQAHNHYA |
| 18928 | ERFHSYPLG |
| 18929 | MAEQMERDF |
| 18930 | DQEAQVVTG |
| 18931 | ACVERLHHK |
| 18932 | NHSLCWDSK |
| 18933 | MYDTMGAWC |
| 18934 | QRNEMSASR |
| 18935 | GHLFNNNEW |
| 18936 | MLPLEMKNL |
| 18937 | ATGTYNLQE |
| 18938 | YVDHWTLGD |
| 18939 | IAQSEADYW |
| 18940 | LINSIVCGD |
| 18941 | LVQKWMRTC |
| 18942 | AAMESCAEI |
| 18943 | DMTLQSVSS |
| 18944 | AEDTPYTIY |
| 18945 | HFQPVFMQP |
| 18946 | LSPWPQTDQ |
| 18947 | AGMYKCQED |
| 18948 | PHAETHWYT |
| 18949 | YPDTADIFW |
| 18950 | VTMIHNAFH |
| 18951 | DLIVATEWF |
| 18952 | HSMMPGWPS |
| 18953 | IWITCPYAA |
| 18954 | PYGPIEYDE |
| 18955 | ATMNLSWGL |
| 18956 | NGVGRSGDN |
| 18957 | RCADNPYFR |
| 18958 | WERCLELNQ |
| 18959 | SNVACIQAF |
| 18960 | INYISFVDR |
| 18961 | LEIGGKTRT |
| 18962 | YDYWMQAPT |
| 18963 | QSAHPEPMC |
| 18964 | ASMHEETCL |
| 18965 | DMIDPVRHG |
| 18966 | ADRSNSSCF |
| 18967 | AEYSTNLHS |
| 18968 | AFSYCMAHM |
| 18969 | AIDYKTDWT |
| 18970 | AKACHLWGK |
| 18971 | ALQMAYEWS |
| 18972 | ANDIAEHWG |
| 18973 | ANVCIITHC |
| 18974 | APFMMQCFS |
| 18975 | APYPSWPTY |
| 18976 | AQWRIMFIA |
| 18977 | ARDCWWSDG |
| 18978 | ARDRPHRGS |
| 18979 | ASCRCLNWH |
| 18980 | ASQRSGRQP |
| 18981 | ATEHVRSWW |
| 18982 | ATIMFIEIS |
| 18983 | ATKCVLGLG |
| 18984 | ATMYMERAD |
| 18985 | ATYEHSCTK |
| 18986 | AVNHAQCGF |
| 18987 | CARLGGRGN |
| 18988 | CCTILNMSG |
| 18989 | CFEFLWEHE |
| 18990 | CLVRIQKDH |
| 18991 | CQAIINADT |
| 18992 | CSTMPKLPE |
| 18993 | CTMSPGYDQ |
| 18994 | CVVMAVDNT |
| 18995 | CWGWAPPDD |
| 18996 | CWPQSCKPE |
| 18997 | DGENMDEMA |
| 18998 | DIGRFIAYY |
| 18999 | DIKLVEAYA |
| 19000 | DKDEFQMAP |
| 19001 | DNDMYHSMQ |
| 19002 | DNNFKDKEE |
| 19003 | DSFYPQLQN |
| 19004 | DSQLISTLA |
| 19005 | DTINWIASQ |
| 19006 | DTLKSDNYI |
| 19007 | DTRGKAFFP |
| 19008 | DVITVYWDS |
| 19009 | DVTDDQIHM |
| 19010 | DVVKSWQMG |
| 19011 | DYSLLWMYE |
| 19012 | ECAFIHVDE |
| 19013 | ECMQYFPEW |
| 19014 | EDRLIVRMD |
| 19015 | EHITEQDLW |
| 19016 | EIGMEYGHS |
| 19017 | EINNAIASR |
| 19018 | EIQQMCVKW |
| 19019 | EIYMCKEGN |
| 19020 | EMSWKMKPT |
| 19021 | ENHPPWLHC |
| 19022 | ERDSLWFGK |
| 19023 | ESPQIHIYS |
| 19024 | ETVQDVGLH |
| 19025 | EVSWVPTTK |
| 19026 | EVVNSQFNL |
| 19027 | EYEWVDEPQ |
| 19028 | EYFKLRQFG |
| 19029 | EYSNETHQE |
| 19030 | FEADNTMVH |
| 19031 | FGHFITKKM |
| 19032 | FIKLTTLPS |
| 19033 | FLEAMQRTP |
| 19034 | FLQYSMSGN |
| 19035 | FMDECLRVD |
| 19036 | FNSSEQQQM |
| 19037 | FSHKISVWC |
| 19038 | FTHWVNEQL |
| 19039 | FVKVVWGHK |
| 19040 | GDNMDQTQG |
| 19041 | GFKKFHFTF |
| 19042 | GKERFHRID |
| 19043 | GMSLKNYEE |
| 19044 | GNYHLDQDE |
| 19045 | GPNGDVARY |
| 19046 | GQQTMEMSY |
| 19047 | GTMHALKDE |
| 19048 | GTQSERDIQ |
| 19049 | GVEFVPIGN |
| 19050 | HCIFKDPKI |
| 19051 | HEVDRPAPC |
| 19052 | HITNYGYCG |
| 19053 | HNPATECRC |
| 19054 | HPLTQMVLE |
| 19055 | HPLTQMVLG |
| 19056 | HRRRAKVWV |
| 19057 | HTRYWGFAL |
| 19058 | IAGMHEFQQ |
| 19059 | IFILIEWHH |
| 19060 | IKTITYHST |
| 19061 | ILWSQHEVL |
| 19062 | INISTPVNC |
| 19063 | ISHQCHQIH |
| 19064 | ISTQMSWDG |
| 19065 | ITFCCRAAK |
| 19066 | ITQYTTYEG |
| 19067 | IVAEHDPGF |
| 19068 | IVMPALMCK |
| 19069 | IYKLFQHGM |
| 19070 | IYRLFQHGM |
| 19071 | KADNRNWSC |
| 19072 | KAQPSEING |
| 19073 | KCLPYMKMF |
| 19074 | KCTAEKAQC |
| 19075 | KCTVEKAQC |
| 19076 | KEREERRFW |
| 19077 | KKYFPTGIG |
| 19078 | KLDYHGVRQ |
| 19079 | KMVQVEHYT |
| 19080 | KVMTAEIAD |
| 19081 | LACACQDAP |
| 19082 | LDGSCYHWG |
| 19083 | LKATCTAMD |
| 19084 | LTTVYNWQH |
| 19085 | LVEMGCEPR |
| 19086 | LWTMDTVRP |
| 19087 | LYVGWMNCL |
| 19088 | MCYHTGHFA |
| 19089 | MGGMIVMCQ |
| 19090 | MHCRTFSHN |
| 19091 | MHMRTNEGS |
| 19092 | MILTEGIIE |
| 19093 | MLHMMPWID |
| 19094 | MMAMKQRFY |
| 19095 | MMGGKFGTT |
| 19096 | MNNEVWKAS |
| 19097 | MNQHWPPQI |
| 19098 | MPLSMKAKK |
| 19099 | MSDTMDMGE |
| 19100 | MSIMCCSDC |
| 19101 | MSNVCICTG |
| 19102 | MTGHNESFT |
| 19103 | MTVAGQSRD |
| 19104 | MTWTRNSGT |
| 19105 | MVDREKKKE |
| 19106 | MVSSETGSH |
| 19107 | MWKRHCGMG |
| 19108 | MWMQMLSAM |
| 19109 | MYCLEFMND |
| 19110 | MYTGMRCWH |
| 19111 | NAPNDLKNY |
| 19112 | NASCQYRGA |
| 19113 | NASCQYRGV |
| 19114 | NATHMYNAH |
| 19115 | NGCIVAEAA |
| 19116 | NGYIVHACQ |
| 19117 | NGYQARTKS |

TABLE 78
Sequences of the 581 to 589 Region in AAV5
VP1 Capsid Polypeptide that Drive Lymph
Node Tissue Tropism

| SEQ ID | 581-589 |
|---|---|
| NO | Sequence |
| 21118 | AVDCHNSWI |
| 21119 | VQQYHITQE |
| 21120 | AAAKWDDVW |
| 21121 | AACKEKRNN |
| 21122 | AACTMAVAR |
| 21123 | AACYDVNKA |
| 21124 | AADLKGYIG |
| 21125 | AAELNSSQS |
| 21126 | AAENCQCPF |
| 21127 | AAEWKVGQA |
| 21128 | AAGEEWSAL |
| 21129 | AAMSCADAS |
| 21130 | AANESTHDC |
| 21131 | AAPYLTGRD |
| 21132 | AASPITVLK |
| 21133 | AASQMMNHG |
| 21134 | AATMNGQCM |
| 21135 | AAVGYFHTS |
| 21136 | AAVMESFWR |
| 21137 | AAVMSLCTV |
| 21138 | ACATTNFYD |
| 21139 | ACDPSHKHQ |
| 21140 | ACDPYSINF |
| 21141 | ACETWQWRE |
| 21142 | ACEWWFNKE |
| 21143 | ACIMQMTQD |
| 21144 | ACKLLDSHL |
| 21145 | ACMPICMSA |
| 21146 | ACTYRMTWH |
| 21147 | ACVDLVVDK |
| 21148 | ACVKHLYDW |
| 21149 | ADAWETPYP |
| 21150 | ADCHTQNVA |
| 21151 | ADCRYNLAC |
| 21152 | ADCSDQMSQ |
| 21153 | ADFDGILWY |
| 21154 | ADIQFARMP |
| 21155 | ADLDINQYW |
| 21156 | ADLHKRHNW |
| 21157 | ADNLTPHQW |
| 21158 | AEHKWPEEL |
| 21159 | AEQFVDVAG |
| 21160 | AESPVAYQQ |
| 21161 | AEWVEQAVH |
| 21162 | AFEHQYCHL |
| 21163 | AFEIVHGAN |
| 21164 | AFETLRNVK |
| 21165 | AFGEFVILA |
| 21166 | AFGHFPGIG |
| 21167 | AFHMMEKWE |
| 21168 | AFISKWVHP |
| 21169 | AFITQVPWY |
| 21170 | AFMNTYVAD |
| 21171 | AFMPWMHTY |
| 21172 | AFMQTICDM |
| 21173 | AFQYSNQAH |
| 21174 | AFRDISKMI |
| 21175 | AFRLLGYSY |
| 21176 | AFRNFTHCP |
| 21177 | AFSLNNCTH |
| 21178 | AFTCCGYEN |
| 21179 | AGDQKYVGC |
| 21180 | AGEEYTTDF |
| 21181 | AGEITKSMF |
| 21182 | AGHRDRAET |
| 21183 | AGLYLTNED |
| 21184 | AGNAMDFCF |
| 21185 | AGNEVQTMP |
| 21186 | AGQYKLIFW |
| 21187 | AGYCSQWRF |
| 21188 | AHAECKFWS |
| 21189 | AHAKWWDYQ |
| 21190 | AHALNEWYA |
| 21191 | AHAMVVNQG |
| 21192 | AHCYVLRHE |
| 21193 | AHELSYSLP |
| 21194 | AHFDYDIFW |
| 21195 | AHGLFMCMN |
| 21196 | AHHEVCWQH |
| 21197 | AHHLQICHT |
| 21198 | AHLWVQGSH |
| 21199 | AHMHTHAIM |
| 21200 | AHNQINDNL |
| 21201 | AHPHFEENG |
| 21202 | AHSWWHRAE |
| 21203 | AIAECIGFT |
| 21204 | AICQLLGDF |
| 21205 | AIEPKEDWM |
| 21206 | AIGHGENYR |
| 21207 | AIHETIAGT |
| 21208 | AIHYLWQDQ |
| 21209 | AIMPVRDGQ |
| 21210 | AIPCEMNAN |
| 21211 | AIQLSQDLM |
| 21212 | AISVMHYDM |
| 21213 | AITDTSCCN |
| 21214 | AITTQGDFY |
| 21215 | AITYQGQGV |
| 21216 | AIVPITFNM |
| 21217 | AIVYKNWAK |
| 21218 | AIWRANEST |
| 21219 | AIYSLDFEQ |
| 21220 | AKFFPHTNT |
| 21221 | AKGLNLDIM |
| 21222 | AKHLFDAAP |
| 21223 | AKHRCMSEW |
| 21224 | AKMWECNPK |
| 21225 | AKNSLYNYH |
| 21226 | AKYWNPELQ |
| 21227 | ALHWWQCLT |
| 21228 | ALINQPDSA |
| 21229 | ALMMYFENQ |
| 21230 | ALTEQCVYG |
| 21231 | ALVLWDMLH |
| 21232 | AMAGPQFSA |
| 21233 | AMAHKGMDA |
| 21234 | AMAQPVKCA |
| 21235 | AMHITQYKT |
| 21236 | AMHQNIFGI |
| 21237 | AMIHIAMMP |
| 21238 | AMQQWVSGC |
| 21239 | AMSLQYKNE |
| 21240 | AMTSRGSYP |
| 21241 | AMVFNMQLG |
| 21242 | AMVSSWLVG |
| 21243 | AMYVMYHGM |
| 21244 | ANDCRGMSE |
| 21245 | ANDLLTMQF |
| 21246 | ANFGSHAAM |
| 21247 | ANHFESSRS |
| 21248 | ANHPNMLIP |
| 21249 | ANMIRKGYG |
| 21250 | ANSFEVCGM |
| 21251 | ANSYYHQYC |
| 21252 | ANVEQAMCA |
| 21253 | ANVPMFRMD |
| 21254 | ANYFPTKFP |
| 21255 | APGCVNQLH |
| 21256 | APLTSYPTL |
| 21257 | APPVFYMPQ |
| 21258 | APTVINMLK |
| 21259 | APWQFNHQW |
| 21260 | AQCPMTWSL |
| 21261 | AQEVTITHE |
| 21262 | AQLLLIVMA |
| 21263 | AQRADWTYL |
| 21264 | AQWPLHRDT |
| 21265 | AQYGRNFAG |
| 21266 | ARMQEPIQV |
| 21267 | ARNANCPRM |
| 21268 | ARQINEHQI |
| 21269 | ARVDDGQMT |
| 21270 | ASANNPVFF |
| 21271 | ASASYEGEQ |
| 21272 | ASAVAKEWD |
| 21273 | ASDGIEKPC |
| 21274 | ASDHLNPFV |
| 21275 | ASDPWNWWP |
| 21276 | ASGWIQLMC |
| 21277 | ASMAYLKGI |
| 21278 | ASNFHSGLY |
| 21279 | ASNFVMEFP |
| 21280 | ASNKICNFK |
| 21281 | ASREIVKHP |
| 21282 | ASTHASESQ |
| 21283 | ASTSQCQFQ |
| 21284 | ASTTIGQEM |
| 21285 | ASVELGGYS |
| 21286 | ASVKDFTVQ |
| 21287 | ASYGDGPMW |
| 21288 | ATAEGEDCT |
| 21289 | ATECAWYHG |
| 21290 | ATFICLTSI |
| 21291 | ATHEYASWD |
| 21292 | ATHPVMLDA |
| 21293 | ATICSGWDR |
| 21294 | ATIGWERYN |
| 21295 | ATIMVCRFE |
| 21296 | ATKELAVQW |
| 21297 | ATKLDFHPT |
| 21298 | ATLTFQQHH |
| 21299 | ATMLRKEVW |
| 21300 | ATMSPECMR |
| 21301 | ATNWEGIDS |
| 21302 | ATNYVTNHD |
| 21303 | ATRVGGSTW |
| 21304 | ATSAHSCSS |
| 21305 | ATSINCNAW |
| 21306 | ATVDYWQAW |
| 21307 | ATWTDSWKV |
| 21308 | ATYCEQYMY |
| 21309 | AVAGVVWDN |
| 21310 | AVAMGGQDD |
| 21311 | AVANWFQHM |
| 21312 | AVCEGEITP |
| 21313 | AVCSKELDH |
| 21314 | AVDHMAMES |
| 21315 | AVDQINMEQ |
| 21316 | AVELSMRAV |
| 21317 | AVEPYCSML |
| 21318 | AVESTAIES |
| 21319 | AVFHQGSGA |
| 21320 | AVITWSQYH |
| 21321 | AVKPSVEPW |
| 21322 | AVMPQGHYG |
| 21323 | AVMTAQGWV |
| 21324 | AVNPFPCQT |
| 21325 | AVQSGIETC |
| 21326 | AVSAAMQMD |
| 21327 | AVSDKPYQH |
| 21328 | AVSFYQYGC |
| 21329 | AVTHKWCEE |
| 21330 | AVTTLLYPI |
| 21331 | AWAKDVMQC |
| 21332 | AWVISQNEN |
| 21333 | AYASSQDSL |
| 21334 | AYENTFPNP |
| 21335 | AYEPFWCWG |
| 21336 | AYHHVDYHW |
| 21337 | AYNQNIGTG |
| 21338 | AYTDLFNQC |
| 21339 | CADFMVSWG |
| 21340 | CADWCNTQP |
| 21341 | CADYQYQQD |
| 21342 | CAIGYWEHD |
| 21343 | CAMMDDRGD |
| 21344 | CAQLFWLPQ |
| 21345 | CASDLMENR |
| 21346 | CASDSLVNG |
| 21347 | CAVNWNVKE |
| 21348 | CAVQFNYCV |
| 21349 | CAYQAMDFF |
| 21350 | CCCNRCHIY |
| 21351 | CCFSTFECY |
| 21352 | CCGDTRCET |
| 21353 | CCGLPARLL |
| 21354 | CCIRSFVCA |
| 21355 | CCKTYQRAG |
| 21356 | CCMTYNNQG |
| 21357 | CCPGNGDIW |
| 21358 | CCPMRHANF |
| 21359 | CCSGNTGMY |
| 21360 | CDHGCWCKG |
| 21361 | CDHVVCMAP |
| 21362 | CDWAAYHCW |
| 21363 | CEGIQVVDF |
| 21364 | CEVTINGAE |
| 21365 | CFAELSNVA |
| 21366 | CGDHYRKGH |
| 21367 | CGHLQQFIL |
| 21368 | CGIGVNMHH |
| 21369 | CGIWNNNAF |
| 21370 | CGRQMEDAA |
| 21371 | CGTQSFIWG |
| 21372 | CGWCGLSKI |
| 21373 | CHAIFCRCA |
| 21374 | CHDGSFNPH |
| 21375 | CHGQYVTWG |
| 21376 | CHHRMHRAQ |
| 21377 | CHMGINRLN |
| 21378 | CHSPFNHWG |
| 21379 | CHWYCSKVP |
| 21380 | CIDEGVSPD |
| 21381 | CIETSDQER |
| 21382 | CIEYTPMGR |
| 21383 | CIGFYMCAG |
| 21384 | CIGVINGMN |
| 21385 | CIHYVQHCQ |
| 21386 | CIILRSNDG |
| 21387 | CILCQPHKW |
| 21388 | CIWKNVPPW |
| 21389 | CKDAYRSHA |
| 21390 | CKFMYLMKE |
| 21391 | CKLTMKRQN |
| 21392 | CKSTHDMAW |
| 21393 | CLHCGTFST |
| 21394 | CLHCHSLQC |
| 21395 | CLMLQSHHT |
| 21396 | CLRAVDNCF |
| 21397 | CMQSESMYC |
| 21398 | CNATWGVFA |
| 21399 | CNAVKNNDM |
| 21400 | CNEVYTLIH |
| 21401 | CNNNYTNWA |
| 21402 | CNNYSMVHR |
| 21403 | CNRLHDTWT |
| 21404 | CNRSEIQFR |
| 21405 | CNVFRDPYL |
| 21406 | CPCFYINYP |
| 21407 | CPETRHVVH |
| 21408 | CPKDCRIVI |
| 21409 | CPLSWIQQE |
| 21410 | CPQPSNRTW |
| 21411 | CPQQIQHYN |
| 21412 | CPTEVREKK |
| 21413 | CQGFGVPWD |
| 21414 | CQHLHTSYW |
| 21415 | CQKALMQLG |
| 21416 | CQTCLQFCD |
| 21417 | CQVEFQMTT |
| 21418 | CRDHYDRSC |
| 21419 | CRFEHQYNC |
| 21420 | CRKLQSIEA |
| 21421 | CRTNGHYYM |
| 21422 | CRVEPVDSD |
| 21423 | CSACTPFSK |
| 21424 | CSADMNSMP |
| 21425 | CSADMQQDI |
| 21426 | CSDASHHFG |
| 21427 | CSEAEGDWY |
| 21428 | CSFNVMNTA |
| 21429 | CSKPDCQWD |
| 21430 | CSMQNNRMY |
| 21431 | CSMYYCWTG |
| 21432 | CSNSQDFGA |
| 21433 | CSTEMQMNC |
| 21434 | CSYQRTEYF |
| 21435 | CTATPEIIH |
| 21436 | CTEININQG |
| 21437 | CTFVRELQC |
| 21438 | CTGDVDQSE |
| 21439 | CTGLAQHWW |
| 21440 | CTGQRHASP |
| 21441 | CTHHYMEQH |
| 21442 | CTKHMDGQA |
| 21443 | CTNDWCRSD |
| 21444 | CTNLISSYC |
| 21445 | CTQVIEECP |
| 21446 | CTTYQDEMN |
| 21447 | CTWWIPLVY |
| 21448 | CTYTELQYM |
| 21449 | CVDQLSLGH |
| 21450 | CVHAISCVG |
| 21451 | CVIGRWLYM |
| 21452 | CVISMAYQW |
| 21453 | CVLKYNHHY |
| 21454 | CVMMQAGGE |
| 21455 | CVSYSGQHD |
| 21456 | CVVNAHQMR |
| 21457 | CVVRAQSAM |
| 21458 | CVVTMSMSD |
| 21459 | CWMFPDYWN |
| 21460 | CWMSFSAYE |
| 21461 | CYESTEGEH |
| 21462 | CYHEYCCNM |
| 21463 | CYKYGPPSQ |
| 21464 | CYMVFMFKA |
| 21465 | CYQDIEAGY |
| 21466 | CYQFKKLTN |
| 21467 | CYQSEKNGY |
| 21468 | CYSGVAMAS |
| 21469 | CYVTKSWDH |
| 21470 | CYWFMYKPK |
| 21471 | DAAQTIQYP |
| 21472 | DAAYSNWFF |
| 21473 | DACKFSSNI |
| 21474 | DADEPWLDR |
| 21475 | DADRAIMHE |
| 21476 | DAEDCHTHK |
| 21477 | DAEGQVCYM |
| 21478 | DAETVDSQF |
| 21479 | DAFLIQELC |
| 21480 | DAGKPQYSI |
| 21481 | DAHDYDSNF |
| 21482 | DAHNYWGWQ |
| 21483 | DAHWVFKTN |
| 21484 | DAIPYKQPT |
| 21485 | DAKFCGQGH |
| 21486 | DALYEMGTT |
| 21487 | DAMPSDRWW |
| 21488 | DANFTMMTD |
| 21489 | DANNKKYHV |
| 21490 | DARHYFGLQ |
| 21491 | DARPDYEMQ |
| 21492 | DARTIVLTH |
| 21493 | DASDCCYSY |
| 21494 | DASLWSVPM |
| 21495 | DASMHGTVP |
| 21496 | DASPTRLVY |
| 21497 | DASVNRVIE |
| 21498 | DATKIPMQQ |
| 21499 | DATQNHFQY |
| 21500 | DAVDVAMAM |
| 21501 | DAVYYLFQA |
| 21502 | DAWHSTCNS |
| 21503 | DAYYDHRGC |
| 21504 | DCGWLAYEW |
| 21505 | DCKCPQPDL |
| 21506 | DCPWKAFHA |
| 21507 | DCRGLSSIF |
| 21508 | DCSVFWIGA |
| 21509 | DCTFTQQQW |
| 21510 | DDKPWVIMA |
| 21511 | DDLQPMERT |
| 21512 | DDVNGAVIM |
| 21513 | DDVNKMRKE |
| 21514 | DDVWMWQWV |
| 21515 | DDYKKWRHP |
| 21516 | DDYKPHCKF |
| 21517 | DECYNLFTV |
| 21518 | DEDDYNMPY |
| 21519 | DEHLITMQT |
| 21520 | DEQWSPTTI |
| 21521 | DEYVPEAYF |
| 21522 | DFAACQDYL |
| 21523 | DFAFENWMG |
| 21524 | DFAWQECWC |
| 21525 | DFEKVSEQA |
| 21526 | DFGCQEQMT |
| 21527 | DFGPRCCFG |
| 21528 | DFGVQKSNE |
| 21529 | DFHDQYVID |
| 21530 | DFIWQQCSN |
| 21531 | DFRFGTRFG |
| 21532 | DFSRMAGGP |
| 21533 | DFTAFKNDM |
| 21534 | DGCPSGNWP |
| 21535 | DGELGKFDI |
| 21536 | DGFDFEKIG |
| 21537 | DGGFQQAEW |
| 21538 | DGHSDLLLS |
| 21539 | DGKHLDTDP |
| 21540 | DGKKTMHET |
| 21541 | DGQLHMFYA |
| 21542 | DGSGSEWHR |
| 21543 | DGTADDKRY |
| 21544 | DGTETIYNM |
| 21545 | DGTYANHMG |
| 21546 | DGTYKCPEP |
| 21547 | DHCNFMAPY |
| 21548 | DHDWLQNMG |
| 21549 | DHEVVQNMP |
| 21550 | DHKLIQMHT |
| 21551 | DHLICQNGP |
| 21552 | DHMWVNWSQ |
| 21553 | DHPFNVKQE |
| 21554 | DHQLTMEGM |
| 21555 | DHSMYATQE |
| 21556 | DICDKNHEG |
| 21557 | DIEFDITCD |
| 21558 | DIGEFWTAH |
| 21559 | DIHRFAEQF |
| 21560 | DIHVITKGI |
| 21561 | DIKQHQLWG |
| 21562 | DILDVVKQG |
| 21563 | DIMINDLYS |
| 21564 | DIQHLTESG |
| 21565 | DIQQHNIAA |
| 21566 | DIQVPLAYR |
| 21567 | DIRRMVASL |
| 21568 | DISMESWMP |
| 21569 | DITETQCGS |
| 21570 | DIYYSMRGR |
| 21571 | DKACMLWDS |
| 21572 | DKCAIEFAS |
| 21573 | DKGALEFWI |
| 21574 | DKGEWQCYK |
| 21575 | DKGFIQHVA |
| 21576 | DKMTLPFSP |
| 21577 | DKPCSICGC |
| 21578 | DKRWSANQW |
| 21579 | DKWPPLEPH |
| 21580 | DLAWQYQPA |
| 21581 | DLEDAHVGL |
| 21582 | DLHGIGVLA |
| 21583 | DLIAGNWPA |
| 21584 | DLPSPRRID |
| 21585 | DLRVMHWHH |
| 21586 | DLVCAQWRE |
| 21587 | DMAAFHYAG |
| 21588 | DMAANHTFP |
| 21589 | DMALYHSNV |
| 21590 | DMDCYTMME |
| 21591 | DMDSPFPWD |
| 21592 | DMDTYYGHG |
| 21593 | DMFTFAHNS |
| 21594 | DMGFVNRHE |
| 21595 | DMIHYPLYG |
| 21596 | DMLMLVTHD |
| 21597 | DMMGFWDKP |
| 21598 | DMNELAWAY |
| 21599 | DMRVGNGYF |
| 21600 | DMSVIQDGA |
| 21601 | DMTRNPEWE |
| 21602 | DNAACIEMS |
| 21603 | DNAEMNSFD |
| 21604 | DNCYSCEAT |
| 21605 | DNEGWLMAE |
| 21606 | DNEVWLSAV |
| 21607 | DNHALEFDC |
| 21608 | DNMQRKWAG |
| 21609 | DNQEPSIRS |
| 21610 | DNQSKESQA |
| 21611 | DNSDFKWHW |
| 21612 | DNVPYCYYQ |
| 21613 | DNYINQEAA |
| 21614 | DPAHTTKIC |
| 21615 | DPDRTPSQH |
| 21616 | DPEEHNNYG |
| 21617 | DPEFQQWTH |
| 21618 | DPIDEVNAQ |
| 21619 | DPNRIDNQY |
| 21620 | DPSEVRARV |
| 21621 | DQCFMTYDT |
| 21622 | DQDNPSCWL |
| 21623 | DQEPNWNGV |
| 21624 | DQFDSHSQG |
| 21625 | DQGCKLRRC |
| 21626 | DQGMGSCKD |
| 21627 | DQHGASTVF |
| 21628 | DQIPMKSMM |
| 21629 | DQKMYVDED |
| 21630 | DQKSVQCYE |
| 21631 | DQLEGGESN |
| 21632 | DQLGLRDNT |
| 21633 | DQQPALDAQ |
| 21634 | DQWSILTFS |
| 21635 | DRAKECQCE |
| 21636 | DRLQCDECS |
| 21637 | DRNTAMTGG |
| 21638 | DRQINWQQE |
| 21639 | DRSDKGCLY |
| 21640 | DRSVHVRAS |
| 21641 | DRVEERAYG |
| 21642 | DRYLFTHWH |
| 21643 | DRYQWAKRH |
| 21644 | DSALQFIDR |
| 21645 | DSAMFGQYK |
| 21646 | DSANVGWCT |
| 21647 | DSAPGKMWP |
| 21648 | DSAPLQSQL |
| 21649 | DSAPPPNVI |
| 21650 | DSDEGSEHI |
| 21651 | DSDLYEGWM |
| 21652 | DSEWLHWHA |
| 21653 | DSGFAMWTP |
| 21654 | DSHFYDQWQ |
| 21655 | DSHPIAHGF |
| 21656 | DSMKGTMFQ |
| 21657 | DSMMHDCHF |
| 21658 | DSMNGVEAS |
| 21659 | DSNEEQRQG |
| 21660 | DSPEQFGLD |
| 21661 | DSQHTAGWP |
| 21662 | DSQLIKQWM |
| 21663 | DSQPMFACV |
| 21664 | DSREAVAVD |
| 21665 | DSRQDHWFE |
| 21666 | DSVHSYEQK |
| 21667 | DSVPNLKAY |
| 21668 | DSYPNQACT |
| 21669 | DTCTTCVSL |
| 21670 | DTCVTAANH |
| 21671 | DTDTRCIPC |
| 21672 | DTEGDHREG |
| 21673 | DTELSMVEP |
| 21674 | DTFFHGAFE |
| 21675 | DTGIWAYVG |
| 21676 | DTHRIIMDD |
| 21677 | DTILEQYGP |
| 21678 | DTIQWDQGI |
| 21679 | DTKMVPYMP |
| 21680 | DTKPENNWI |
| 21681 | DTMHCLSCD |
| 21682 | DTMPTMDLM |
| 21683 | DTQWQEWCE |
| 21684 | DTRDNWLIR |
| 21685 | DTRTPAVWP |
| 21686 | DTSQTSNNT |
| 21687 | DTSRCLAYW |
| 21688 | DTWTLDFEV |
| 21689 | DTYAEIMYG |
| 21690 | DTYPLVIGS |
| 21691 | DVAMTSLIA |
| 21692 | DVCDSIGYC |
| 21693 | DVCEQRNWL |
| 21694 | DVDDSQCEY |
| 21695 | DVDDWFHQS |
| 21696 | DVDHICEGH |
| 21697 | DVDMDTNRG |
| 21698 | DVENHYMGS |
| 21699 | DVEPMKNDF |
| 21700 | DVESRYDCG |
| 21701 | DVFCMLASH |
| 21702 | DVFIQQAYS |
| 21703 | DVFMGARFE |
| 21704 | DVFPCQKTE |
| 21705 | DVFQCEELS |
| 21706 | DVGFNRISR |
| 21707 | DVGIQHRPL |
| 21708 | DVGLFHGGF |
| 21709 | DVGPQKNIV |
| 21710 | DVHMADGNN |
| 21711 | DVILTYDRA |
| 21712 | DVIRTHDGV |
| 21713 | DVKCMQNTF |
| 21714 | DVKGNNGGG |
| 21715 | DVKIVWSGE |
| 21716 | DVKLAGMIH |
| 21717 | DVKPAMNQA |
| 21718 | DVKPGWMGD |
| 21719 | DVKWMESWL |
| 21720 | DVKYLTHQW |
| 21721 | DVLLLEHER |
| 21722 | DVLTNLNGF |
| 21723 | DVNFVGEPE |
| 21724 | DVNSYKLYG |
| 21725 | DVQTIRMEH |
| 21726 | DVRERDSDD |
| 21727 | DVRTEPTIT |
| 21728 | DVRTVTQHG |
| 21729 | DVSQSDWQP |
| 21730 | DVTAQRAWK |
| 21731 | DVTAYSESI |
| 21732 | DVTEDVVSP |
| 21733 | DVTENREVW |
| 21734 | DVTIYWNDY |
| 21735 | DVTLNEGVV |
| 21736 | DVTPEDACV |
| 21737 | DVVALQASK |
| 21738 | DVVANLTSS |
| 21739 | DVVEQFHWT |
| 21740 | DVVIVDCAN |
| 21741 | DVVVSQMAM |
| 21742 | DVWAQSQVL |
| 21743 | DVWLQIDGQ |
| 21744 | DVWWVEGEY |
| 21745 | DWASPKSYV |
| 21746 | DWCMSMVWT |
| 21747 | DWDQQRQYF |
| 21748 | DWGAMWVCG |
| 21749 | DWGHRIFEH |
| 21750 | DWHHPPVDH |
| 21751 | DWKPIYEAS |
| 21752 | DWPIRSAPI |
| 21753 | DYCTGACDC |
| 21754 | DYDVASFYH |
| 21755 | DYEGPIRPN |
| 21756 | DYEHYWHET |
| 21757 | DYKLHPIGY |
| 21758 | DYKMDVMPC |
| 21759 | DYNMMHWAA |
| 21760 | DYQRCTFLP |
| 21761 | EACSNSHEK |
| 21762 | EADCVHRVW |
| 21763 | EADVSLMHC |
| 21764 | EAEAEVIIR |
| 21765 | EAECTWASL |
| 21766 | EAELKTNFM |
| 21767 | EAESGGAYK |
| 21768 | EAEYRNWNP |
| 21769 | EAGMFVHRA |
| 21770 | EAGQWLGSP |
| 21771 | EAHAWEKTE |
| 21772 | EAHEEWSFD |
| 21773 | EAHVNVYKY |
| 21774 | EAKCMHDFS |
| 21775 | EAMIWNNTM |
| 21776 | EANQILSAH |
| 21777 | EANSSQSQF |
| 21778 | EANWEHSCI |
| 21779 | EAQTTGSTW |
| 21780 | EASLIKEDK |
| 21781 | EASSLYSQQ |
| 21782 | EASWSLHGQ |
| 21783 | EATGESFWF |
| 21784 | EAWHPGCCQ |
| 21785 | EAWMCVEAQ |
| 21786 | EAWSMNAPY |
| 21787 | EAYFDTAEC |
| 21788 | ECAPWVVLS |
| 21789 | ECCIGDNMM |
| 21790 | ECEMMDYSK |
| 21791 | ECFATGEFM |
| 21792 | ECHKCSKLN |
| 21793 | ECKLSVCGY |
| 21794 | ECMHAEWQA |
| 21795 | ECMLIGTDG |
| 21796 | ECPDYMRKD |
| 21797 | ECQMHRTFA |
| 21798 | ECRPWGMHE |
| 21799 | ECSESAEAK |
| 21800 | ECTVMQRGA |
| 21801 | ECWFCDIWR |
| 21802 | EDLHQWAQH |
| 21803 | EDMFCPTKA |
| 21804 | EEDFRNEAM |
| 21805 | EEDMQRTVQ |
| 21806 | EEEAGKWMC |
| 21807 | EELEQIWHG |
| 21808 | EESLDVVMK |
| 21809 | EFCYEPDWD |
| 21810 | EFETFWEVG |
| 21811 | EFGTMHYAP |
| 21812 | EFGTSQKDA |
| 21813 | EFGYSPGVS |
| 21814 | EFIHAENWY |
| 21815 | EFKARMVTP |
| 21816 | EFLVNTSWL |
| 21817 | EFMCSCMLN |
| 21818 | EFMTYETNV |
| 21819 | EFNSIMHWW |
| 21820 | EFQWRACFQ |
| 21821 | EFRMASSDA |
| 21822 | EFSDVQRTA |
| 21823 | EFSITMAPW |
| 21824 | EFSKMSGTA |
| 21825 | EFVEKWLWG |
| 21826 | EGAELEWDH |
| 21827 | EGAQLDVMS |
| 21828 | EGDMTTNQY |
| 21829 | EGEELRVAY |
| 21830 | EGEEYTLPS |
| 21831 | EGFLGFGQN |
| 21832 | EGFYLEGDP |
| 21833 | EGKEWIFYE |
| 21834 | EGMKTQQTF |
| 21835 | EGMWPHSAF |
| 21836 | EGQRLTWAQ |
| 21837 | EGRQHFVYA |
| 21838 | EGSQWRTSI |
| 21839 | EGTMSNGWQ |
| 21840 | EGVRLNLYG |
| 21841 | EGWFMKKQD |
| 21842 | EGYEYHTQR |
| 21843 | EHATESWAG |
| 21844 | EHATIENWP |
| 21845 | EHDGVWMYQ |
| 21846 | EHEDTIAHD |
| 21847 | EHFPQQSGD |
| 21848 | EHGPCMSGC |
| 21849 | EHIGCGYWQ |
| 21850 | EHIRFGHYW |
| 21851 | EHKPDGANY |
| 21852 | EHLKLLIAC |
| 21853 | EHMSICDAP |
| 21854 | EHNSEDCIC |
| 21855 | EHPMENTYQ |
| 21856 | EHQFMWQDH |
| 21857 | EHRDHDAWA |
| 21858 | EHVKCNHFF |
| 21859 | EIAGSIMHL |
| 21860 | EIAKWNCEL |
| 21861 | EIAQLQFDQ |
| 21862 | EICAFDVGW |
| 21863 | EICVIECEQ |
| 21864 | EIDYHICSG |
| 21865 | EIEMAVAYA |
| 21866 | EIETSAREQ |
| 21867 | EIFQYMHHH |
| 21868 | EIGIGAPDY |
| 21869 | EIHHGHAVP |
| 21870 | EIIAQKWTQ |
| 21871 | EIIMYEQHM |
| 21872 | EIIPTSMFG |
| 21873 | EIITVRDDE |
| 21874 | EIIYRFVDA |
| 21875 | EILSDDDMP |
| 21876 | EIMHRCHFW |
| 21877 | EIMIQVIRD |
| 21878 | EIMMEHDNK |
| 21879 | EIMWPLAGN |
| 21880 | EINLGHQHN |
| 21881 | EIQMILTAV |
| 21882 | EIRSHDTGH |
| 21883 | EISWQDRDL |
| 21884 | EITFNQYGH |
| 21885 | EITWESNIA |
| 21886 | EIVDLTVME |
| 21887 | EIVEHDYRE |
| 21888 | EIVYMMCPC |
| 21889 | EKAGNLERK |
| 21890 | EKDQYKMGS |
| 21891 | EKEQFSHKG |
| 21892 | EKGMIDLVY |
| 21893 | EKGMNMHAG |
| 21894 | EKHQLSVQQ |
| 21895 | EKQWLSRQD |
| 21896 | EKSGYHFSC |
| 21897 | EKVICLYRG |
| 21898 | EKVTLNMDM |
| 21899 | EKYMNFMCP |
| 21900 | ELADQNQIQ |
| 21901 | ELAWIWQIH |
| 21902 | ELCCNDMFK |
| 21903 | ELERTVKEF |
| 21904 | ELGYSEKVC |
| 21905 | ELLTCIECG |
| 21906 | ELNFCWQQR |
| 21907 | ELYMNNHTE |
| 21908 | EMAELNQHV |
| 21909 | EMDQVPWGF |
| 21910 | EMEFPDMCM |
| 21911 | EMEWQDSWW |
| 21912 | EMGFGKQGM |
| 21913 | EMGMPSYMK |
| 21914 | EMKEAWLSA |
| 21915 | EMMVLCENE |
| 21916 | EMPTTCKNC |
| 21917 | EMQCYQTYH |
| 21918 | EMSECSITA |
| 21919 | EMTPQQFWH |
| 21920 | EMWICHFGG |
| 21921 | EMWSRGMVN |
| 21922 | EMYPFSKQL |
| 21923 | ENAEIKDHM |
| 21924 | ENAIKHHDS |
| 21925 | ENAQRHNYA |
| 21926 | ENDAEVPDH |
| 21927 | ENEAMAKWF |
| 21928 | ENFMLRPCN |
| 21929 | ENHASAHVR |
| 21930 | ENHIEFWWP |
| 21931 | ENLESRLQH |
| 21932 | ENLHCDCLY |
| 21933 | ENLRMGKWE |
| 21934 | ENVKLDQCD |
| 21935 | ENWHLVAHR |
| 21936 | ENYCQCCLE |
| 21937 | ENYRHHTGP |
| 21938 | EPEWLGHEE |
| 21939 | EPGPMQCWD |
| 21940 | EPGQFFHDC |
| 21941 | EPLDNVGCG |
| 21942 | EPMNGKVME |
| 21943 | EPNAQMTAA |
| 21944 | EPQEVENMT |
| 21945 | EPTLVGEVP |
| 21946 | EPTNIWAVH |
| 21947 | EPTSATWVY |
| 21948 | EPVHLPEWM |
| 21949 | EQFGCGQWL |
| 21950 | EQKMKQIAQ |
| 21951 | EQKSTQTFH |
| 21952 | EQLDSHWWK |
| 21953 | EQLDVKYWH |
| 21954 | EQLIEWSSH |
| 21955 | EQNMLDADC |
| 21956 | EQQPMASFT |
| 21957 | EQQYLAQGK |
| 21958 | EQTATMRWD |
| 21959 | EQVYYWNTP |
| 21960 | EQWYDEIPH |
| 21961 | EQYYFNQLR |
| 21962 | ERFYQHAAK |
| 21963 | ERGYNMAQH |
| 21964 | ERIQWTHMG |
| 21965 | ERTAPSSRG |
| 21966 | ERTMVHQYW |
| 21967 | ERVDQENCH |
| 21968 | ERVTDEHEI |
| 21969 | ERYHMRWEP |
| 21970 | ESANYMNLF |
| 21971 | ESCYMMMNW |
| 21972 | ESGMLFYFM |
| 21973 | ESKAHAQTP |
| 21974 | ESKTTSCAF |
| 21975 | ESLRAENMP |
| 21976 | ESMGFEECG |
| 21977 | ESMKLIQCD |
| 21978 | ESMNIMTYK |
| 21979 | ESNDNGNQW |
| 21980 | ESNPVCVLK |
| 21981 | ESRCAWLSD |
| 21982 | ESRKEIGYA |
| 21983 | ESRRHHWAH |
| 21984 | ESSDISNYL |
| 21985 | ESTLLLCHG |
| 21986 | ESTNLQCVL |
| 21987 | ESVTSLDQQ |
| 21988 | ESYETQLAC |
| 21989 | ESYFSETQP |
| 21990 | ESYIVFDNT |
| 21991 | ESYKCIRDC |
| 21992 | ETAHYSMRE |
| 21993 | ETANQEWER |
| 21994 | ETASKNVQT |
| 21995 | ETCILAVDG |
| 21996 | ETDEIYIVG |
| 21997 | ETDQQYDQY |
| 21998 | ETEEIVNMA |
| 21999 | ETEGYLFSP |
| 22000 | ETEPPDTQG |
| 22001 | ETESMMFCN |
| 22002 | ETHTYDDCS |
| 22003 | ETIAMQYRC |
| 22004 | ETKCYAMLG |
| 22005 | ETKIYDCNG |
| 22006 | ETKLAIDEF |
| 22007 | ETKSVLDEH |
| 22008 | ETMCPKGPL |
| 22009 | ETMNQIGSC |
| 22010 | ETNLFEHST |
| 22011 | ETPGTKQCS |
| 22012 | ETQRDRHTN |
| 22013 | ETRDYLEAD |
| 22014 | ETRQMDGNN |
| 22015 | ETSMRFESL |
| 22016 | ETSRMNVDA |
| 22017 | ETTCYAYME |
| 22018 | ETVERQCEQ |
| 22019 | EVAETSHSY |
| 22020 | EVANGKGDH |
| 22021 | EVCLGQRYA |
| 22022 | EVDRHLGMQ |
| 22023 | EVEACKYEY |
| 22024 | EVECTHRGE |
| 22025 | EVEVWSVYH |
| 22026 | EVFPYGCQN |
| 22027 | EVGMIVHPD |
| 22028 | EVGTCYFSK |
| 22029 | EVHKNDKMH |
| 22030 | EVHSMDTQW |
| 22031 | EVIQTSDNR |
| 22032 | EVKFEMLCQ |
| 22033 | EVKHKQDWA |
| 22034 | EVKNKGGFC |
| 22035 | EVKPCLVVQ |
| 22036 | EVKTRMCAG |
| 22037 | EVLKAQREV |
| 22038 | EVLLSTDEA |
| 22039 | EVLPYEWCE |
| 22040 | EVMIRKLDM |
| 22041 | EVMYSERTW |
| 22042 | EVNMEMQLA |
| 22043 | EVQGEKCLN |
| 22044 | EVQQFHRDQ |
| 22045 | EVQRWSGYH |
| 22046 | EVRGAEANH |
| 22047 | EVSKLATTY |
| 22048 | EVSQFGIMN |
| 22049 | EVTFWTCEG |
| 22050 | EVTIRSHYQ |
| 22051 | EVTLIPDSW |
| 22052 | EVVHSMSDG |
| 22053 | EVVNVPMRT |
| 22054 | EVYRNTMKC |
| 22055 | EWAGKQTDM |
| 22056 | EWAYMGSYP |
| 22057 | EWDDTSNSG |
| 22058 | EWHFPKAMN |
| 22059 | EWNDYPSWY |
| 22060 | EWRHVELHY |
| 22061 | EYAGHHCFY |
| 22062 | EYCMCMWGA |
| 22063 | EYCNVMIPC |
| 22064 | EYDDVPEPQ |
| 22065 | EYEQDCRFQ |
| 22066 | EYHDKHWAG |
| 22067 | EYHTEWIGM |
| 22068 | EYIGEELHK |
| 22069 | EYILWHWCT |
| 22070 | EYISTQADA |
| 22071 | EYKYYTYIF |
| 22072 | EYMATEHGN |
| 22073 | EYMHYYGSI |
| 22074 | EYPLESTYK |
| 22075 | EYPLPVTLQ |
| 22076 | EYSQLHPSP |
| 22077 | EYTCRLLYD |
| 22078 | EYWIGQQSC |
| 22079 | EYWQISAWS |
| 22080 | FACEIQYSD |
| 22081 | FAEDCQRFD |
| 22082 | FALNGIEKW |
| 22083 | FASRYDGHH |
| 22084 | FAVFCWAER |
| 22085 | FAYCESEEQ |
| 22086 | FCDACLGGY |
| 22087 | FCELYFHCA |
| 22088 | FCLDPNFYY |
| 22089 | FCTGASHAH |
| 22090 | FDDCTWQGP |
| 22091 | FDHATMQFG |
| 22092 | FDRCNESYC |
| 22093 | FEPHGCQIE |
| 22094 | FEQADMMQM |
| 22095 | FEQYIRNIK |
| 22096 | FERLFMGWT |
| 22097 | FEVMRAIRC |
| 22098 | FFTKISWSW |
| 22099 | FGTAVMVWE |
| 22100 | FGWDAYCRV |
| 22101 | FHQYHYQAM |
| 22102 | FHWWGAVVK |
| 22103 | FICDVQMGD |
| 22104 | FIEFYMGEQ |
| 22105 | FIEGEMHIR |
| 22106 | FIGLQWSYP |
| 22107 | FIPISHQHP |
| 22108 | FIRSYNVEC |
| 22109 | FIRVSWWLA |
| 22110 | FIYTDFRMT |
| 22111 | FKEELMKYQ |
| 22112 | FKFEQENNS |
| 22113 | FKKPMVCHV |
| 22114 | FKLVSGDQW |
| 22115 | FKQIDHYPR |
| 22116 | FMAELKTDD |
| 22117 | FMKSKLETV |

TABLE 79
Sequences of the 581 to 589 Region in AAV5
VP1 Capsid Polypeptide that Drive Mammary
Gland Tissue Tropism

| SEQ ID | 581-589 |
|---|---|
| NO | Sequence |
| 24118 | FGDHLRVMY |
| 24119 | LPTQYQQQN |
| 24120 | TKKIIESSS |
| 24121 | IMYLKRCCT |
| 24122 | TSDQCGNED |
| 24123 | GQFPHYGIF |
| 24124 | YTELMKKAC |
| 24125 | LGMHYKDNC |
| 24126 | KMGTMDFNH |
| 24127 | QWDDEWDMD |
| 24128 | THACMYHEY |
| 24129 | LEQCHDHCA |
| 24130 | TKVNCVYYG |
| 24131 | QELGVNCAA |
| 24132 | QKDWGPRVT |
| 24133 | APMTCKEFL |
| 24134 | ASWFPVLFP |
| 24135 | CTYAAMMSY |
| 24136 | CVQCECNQN |
| 24137 | DMFRPDVRC |
| 24138 | DTDNKPHFA |
| 24139 | DTHIFIRHA |
| 24140 | EFHFKCRTD |
| 24141 | EHHAMMLHE |
| 24142 | FSHMPTSGG |
| 24143 | FVCPRAFNM |
| 24144 | FWMNCWVIV |
| 24145 | GQYIAVNYS |
| 24146 | HYEMPQAMQ |
| 24147 | KCGNEGRMS |
| 24148 | LQKGWANCE |
| 24149 | LSTLITMMD |
| 24150 | LTDFHPKML |
| 24151 | MAMKLIEGN |
| 24152 | NFCKQGNTC |
| 24153 | NIRCGNDAS |
| 24154 | NQQPGQHSQ |
| 24155 | NVVCVRYKS |
| 24156 | QATMDAWVH |
| 24157 | QIPKAWNRY |
| 24158 | QTTTNDSSQ |
| 24159 | SADRTAMFH |
| 24160 | SARTDCQCK |
| 24161 | SDSKVQWGM |
| 24162 | SEDHFGFWV |
| 24163 | SHWFFHFDL |
| 24164 | STSWLNDLT |
| 24165 | TEFSQQYKC |
| 24166 | VAFEYGERA |
| 24167 | VCGWWHQYW |
| 24168 | VHTEMSNID |
| 24169 | VTQRPLHHS |
| 24170 | WLYLAFAQN |
| 24171 | WWKPCNCQY |
| 24172 | YATQMQHLE |
| 24173 | YMGSTNIWM |
| 24174 | GVGPSHGER |
| 24175 | GVMHHGIFD |
| 24176 | AISMYWSSW |
| 24177 | AVQCYIHPS |
| 24178 | CSTIHAGHP |
| 24179 | DIGMYWSSW |
| 24180 | DISMCWSSW |
| 24181 | DISMDWSSW |
| 24182 | DISMYWGSW |
| 24183 | DISMYWSGW |
| 24184 | DISMYWSSL |
| 24185 | DISMYWSSW |
| 24186 | DMSMYWSSW |
| 24187 | GISMYWSSW |
| 24188 | KPMINEIVY |
| 24189 | LHALINQGS |
| 24190 | LHALVNQGS |
| 24191 | LHALVNQGY |
| 24192 | LRALVNQGS |
| 24193 | MCLRDHNGL |
| 24194 | MPMINEIVY |
| 24195 | MYLCDHNGL |
| 24196 | MYLRDDNGL |
| 24197 | MYLRDHDGL |
| 24198 | MYLRDHNCL |
| 24199 | MYLRDHNDL |
| 24200 | MYLRDHNGL |
| 24201 | MYLRDHNSL |
| 24202 | MYLRDHNVL |
| 24203 | MYLRDHSGL |
| 24204 | MYLRDHTGL |
| 24205 | MYLRDRNGL |
| 24206 | MYLRGHNGL |
| 24207 | MYLRNHNGL |
| 24208 | NISMYWSSW |
| 24209 | QCYAIAHLI |
| 24210 | RCCAIAHLI |
| 24211 | RCYAIADLI |
| 24212 | RCYAIAHLI |
| 24213 | RCYAIAHLL |
| 24214 | RCYAIAHLV |
| 24215 | RCYAIAHVI |
| 24216 | RCYAIAHWI |
| 24217 | RCYAIALLI |
| 24218 | RCYAIAPLI |
| 24219 | RCYAIARLI |
| 24220 | RCYAIGHLI |
| 24221 | RCYAISHLI |
| 24222 | RCYAITHLI |
| 24223 | RCYAIVHLI |
| 24224 | RCYAVAHLI |
| 24225 | RCYDIAHLI |
| 24226 | RCYSIAHLI |
| 24227 | RCYTIAHLI |
| 24228 | RGYAIAHLI |
| 24229 | RWYAIAHLI |
| 24230 | SIQCYIHPS |
| 24231 | SVQCCIHPS |
| 24232 | SVQCYIHPS |
| 24233 | SVQCYIRPS |
| 24234 | SVQCYMHPS |
| 24235 | SVQCYVHPS |
| 24236 | SVQFYIHPS |
| 24237 | TPMIDEIVY |
| 24238 | TPMINEIFY |
| 24239 | TPMINEIVC |
| 24240 | TPMINEIVY |
| 24241 | TPMINEMVY |
| 24242 | TPMINEVVY |
| 24243 | TPMINGIVY |
| 24244 | TPMISEIVY |
| 24245 | TPMVNEIVY |
| 24246 | VYLRDHNGL |
| 24247 | YSTIHAGHP |
| 24248 | RVTIMFTGT |
| 24249 | GTRINTCNG |
| 24250 | AVGSLRTFQ |
| 24251 | CNTPYEFWA |
| 24252 | DLRLMACCG |
| 24253 | RRRFGISGL |
| 24254 | VTYYSMTQQ |
| 24255 | MASRKSMID |
| 24256 | GMVELGSCW |
| 24257 | TVTTTKWRD |
| 24258 | QHTQQLVQN |
| 24259 | SGNMYESGE |
| 24260 | QDFRMNAGA |
| 24261 | GLSMVLHFV |
| 24262 | AMTRCKAGH |
| 24263 | GSNRNPIMM |
| 24264 | ASAGRNWTA |
| 24265 | TYWTKQSEV |
| 24266 | CIWSSPDQP |
| 24267 | NYLQMCWFN |
| 24268 | TTYPHNTHG |
| 24269 | TCTNSEPHP |
| 24270 | ECQYEMNDD |
| 24271 | THAQMSLRD |
| 24272 | KYASMDIYS |
| 24273 | AQSKSLMRC |
| 24274 | TVMQCGTMP |
| 24275 | TAFQSLQQM |
| 24276 | HMDEVCSKD |
| 24277 | YVSLCNDGN |
| 24278 | RVEYVTDSH |
| 24279 | MCQNVEKEY |
| 24280 | NSTPMACME |
| 24281 | TSEPHCSYA |
| 24282 | DPGWYGLAP |
| 24283 | CWGPIMHPF |
| 24284 | LSVQAQWDS |
| 24285 | IAKIGNCVW |
| 24286 | KAVSYHHDG |
| 24287 | SLTQYQWRG |
| 24288 | MATCYMYAD |
| 24289 | APFFTFTNH |
| 24290 | TRMQAVDHE |
| 24291 | FWMQTEHGT |
| 24292 | GCEEVGRCQ |
| 24293 | IKHITRNTY |
| 24294 | WKWYGLPHH |
| 24295 | ESTFWQHTF |
| 24296 | SQMKYAHNQ |
| 24297 | AEVPYVMQQ |
| 24298 | TCGHTRFFI |
| 24299 | NTSGELIMP |
| 24300 | DRVNLMWKD |
| 24301 | AVHTADVCC |
| 24302 | EFVLGCQGI |
| 24303 | DVKAFMGGV |
| 24304 | TTSQNSEAM |
| 24305 | YASSCQNAM |
| 24306 | QSMVACYTI |
| 24307 | MGYAKHDSH |
| 24308 | SSRMQMQGP |
| 24309 | SQIQIRQAD |
| 24310 | VHHKTNRSY |
| 24311 | RVNGSTCRH |
| 24312 | NGNHYNAMR |
| 24313 | WTTSIAVDT |
| 24314 | FTEPMCMQG |
| 24315 | DVKSWAVCD |
| 24316 | AYHCYMSWM |
| 24317 | RYPCCQSPF |
| 24318 | AHAWLQHWA |
| 24319 | HQCNCLLSW |
| 24320 | TNFPTSMHG |
| 24321 | QHLMTFSHE |
| 24322 | QRNEMSASR |
| 24323 | WDGWVTTHF |
| 24324 | PAHINDQYM |
| 24325 | VLKSNLTGM |
| 24326 | ATGTYNLQE |
| 24327 | LINSIVCGD |
| 24328 | ACHTGQSGM |
| 24329 | DHLSNDAEM |
| 24330 | SECEVLCSL |
| 24331 | VFNLMLQDK |
| 24332 | ILINDREWG |
| 24333 | QWFTKGVGE |
| 24334 | NVMGTQAKE |
| 24335 | YSDPKNMSI |
| 24336 | LEIGGKTRT |
| 24337 | ASMHEETCL |
| 24338 | WERCLELNQ |
| 24339 | MPWLDKPPC |
| 24340 | ATMNLSWGL |
| 24341 | WVHPYSYCA |
| 24342 | QSRCVDNTV |
| 24343 | NRIQMTDFQ |
| 24344 | SNVACIQAF |
| 24345 | INYISFVDR |
| 24346 | EVRIQCKID |
| 24347 | SVLHVQALA |
| 24348 | CWILHHRCS |
| 24349 | KMAGIGCWY |
| 24350 | LCAVCGDCD |
| 24351 | HSMMPGWPS |
| 24352 | DQEAQVVTG |
| 24353 | ISLVWMHYP |
| 24354 | AACNGLEYQ |
| 24355 | AADCYHWFR |
| 24356 | AAEWDRFAE |
| 24357 | AAGLQVDSW |
| 24358 | AAMASAFVY |
| 24359 | AAMLGWESE |
| 24360 | AASPKTMNE |
| 24361 | ACDRFMAMY |
| 24362 | ACSVSICTE |
| 24363 | ACYDYYQQV |
| 24364 | ACYSYEFEE |
| 24365 | ADCFIFAEY |
| 24366 | AELEIMWDD |
| 24367 | AFGVYPSVS |
| 24368 | AFNHVHEGT |
| 24369 | AFTIEDDWW |
| 24370 | AGVQENVSV |
| 24371 | AILTVCIGA |
| 24372 | AINMSGSGN |
| 24373 | AKNDNLCAD |
| 24374 | AKRQKVVLS |
| 24375 | AKWRGTLIN |
| 24376 | ALCDYMSWH |
| 24377 | AMEYRSWHT |
| 24378 | ANCIRPFTS |
| 24379 | ANCKQDHCH |
| 24380 | ANICTIESD |
| 24381 | ANIRGRSMK |
| 24382 | ANQVTGSWK |
| 24383 | ANTQYPAHM |
| 24384 | ANWPMACDQ |
| 24385 | APFRKQMPG |
| 24386 | APWHSNDMR |
| 24387 | AQNVLDTVQ |
| 24388 | AQNVLGTVQ |
| 24389 | AQVSTLNGM |
| 24390 | AREDPCAPR |
| 24391 | ARGWDYKCP |
| 24392 | ASERLYYCW |
| 24393 | ASILQFTVD |
| 24394 | ASKPTRKVT |
| 24395 | ASQYQQSLS |
| 24396 | ATAINQRQA |
| 24397 | ATEQCLNYL |
| 24398 | ATIRQTLLN |
| 24399 | ATTCITSDD |
| 24400 | ATTKGMSGW |
| 24401 | ATTLINQEW |
| 24402 | AVGHFYTNS |
| 24403 | AVHTADVFC |
| 24404 | AVIFMSVCE |
| 24405 | AVTQQVDEN |
| 24406 | AWCNIDMST |
| 24407 | AWSCIMSYD |
| 24408 | AYENFHWYD |
| 24409 | AYHDSGHEW |
| 24410 | AYYQLQMSS |
| 24411 | CADVPITMA |
| 24412 | CAEQWAAML |
| 24413 | CAHWTYDCY |
| 24414 | CAHWYYEQR |
| 24415 | CANKEHVCH |
| 24416 | CANWKDHGY |
| 24417 | CAQHRDTAF |
| 24418 | CASLKVFDS |
| 24419 | CASTYTGIN |
| 24420 | CATAMFRYQ |
| 24421 | CATKSMMYG |
| 24422 | CAWLSQTAS |
| 24423 | CCMPLQSFG |
| 24424 | CEGFEMRVE |
| 24425 | CEGWGTYCI |
| 24426 | CEHGNENWP |
| 24427 | CFGFVNSVN |
| 24428 | CFMWNWHHE |
| 24429 | CFRMYDLLS |
| 24430 | CFTNNVVWT |
| 24431 | CGCSEVTPH |
| 24432 | CGLIMEMCW |
| 24433 | CGWWFNAAE |
| 24434 | CHAEYSHEQ |
| 24435 | CHFQYQNQA |
| 24436 | CHLLLTDCY |
| 24437 | CHMMDWQCG |
| 24438 | CHMQFMEAG |
| 24439 | CHTEYSHEQ |
| 24440 | CIDLRLAWD |
| 24441 | CIEWWGMMG |
| 24442 | CIIPKMKER |
| 24443 | CILHRYVIE |
| 24444 | CILLKKCMA |
| 24445 | CINADGKYS |
| 24446 | CIQAVIGGT |
| 24447 | CIRLTEYLG |
| 24448 | CIRTMNGSW |
| 24449 | CISRFDHKQ |
| 24450 | CKQWTNQAH |
| 24451 | CKVHKNWDR |
| 24452 | CLGSTWVAA |
| 24453 | CMCLKKCYY |
| 24454 | CMQQYDNGQ |
| 24455 | CMREQMNAC |
| 24456 | CNKDINQSC |
| 24457 | CNWLFMTQW |
| 24458 | CPGPMMQMA |
| 24459 | CPKPIGCGG |
| 24460 | CQDFRHECA |
| 24461 | CQQRIIMQA |
| 24462 | CQRKWEKQE |
| 24463 | CRTELGSMY |
| 24464 | CSAKVPMPT |
| 24465 | CSAQIKAAC |
| 24466 | CSCAMYNFH |
| 24467 | CSDQYQTQN |
| 24468 | CSGRLGSLY |
| 24469 | CSHHLMTFG |
| 24470 | CSHNFQDQK |
| 24471 | CSIWEDKSG |
| 24472 | CSMECRNEL |
| 24473 | CSTTMNVKT |
| 24474 | CSVITQTTG |
| 24475 | CTDKLLNAG |
| 24476 | CTDRMVKAS |
| 24477 | CTENYHQQY |
| 24478 | CTGQYMGYI |
| 24479 | CTHQQSIAV |
| 24480 | CTLKQNMPA |
| 24481 | CTQLSVNMQ |
| 24482 | CTQSFVLYQ |
| 24483 | CTVFSGRTA |
| 24484 | CTVRQWHKP |
| 24485 | CVAIDMSVA |
| 24486 | CVEYLKQLY |
| 24487 | CVGDREPWG |
| 24488 | CVGINLDHH |
| 24489 | CVGKQIMAE |
| 24490 | CVIHPPNFD |
| 24491 | CVLYYNQHC |
| 24492 | CVQGQYYQE |
| 24493 | CVQHNLVGY |
| 24494 | CVQTMNGKS |
| 24495 | CVYREIKTA |
| 24496 | CWAIFRLQS |
| 24497 | CWAQLNTDT |
| 24498 | CWEMMKTQE |
| 24499 | CWNIYNACD |
| 24500 | CWSSWQKRD |
| 24501 | CYDGWWCPS |
| 24502 | CYKFVGAAD |
| 24503 | CYRDVRARG |
| 24504 | CYVYWTTCP |
| 24505 | DAAQFGKHT |
| 24506 | DACWGHEYA |
| 24507 | DAGTNSFCG |
| 24508 | DAGVTTYGG |
| 24509 | DASKVKLHH |
| 24510 | DASSYLHLC |
| 24511 | DCAKFPFMI |
| 24512 | DCEDIKKWE |
| 24513 | DCGQFSIIE |
| 24514 | DDHWDWLCY |
| 24515 | DDLGAGRSD |
| 24516 | DDLSMTNDC |
| 24517 | DEEFYPPAW |
| 24518 | DEFIWLEMH |
| 24519 | DETVFRNPV |
| 24520 | DFEYFSTKD |
| 24521 | DGFQYDAHC |
| 24522 | DGVQVDWGR |
| 24523 | DHKEYMDKG |
| 24524 | DHRQWKVLA |
| 24525 | DICHYIRNE |
| 24526 | DIMRYINTA |
| 24527 | DIRTSVSDS |
| 24528 | DITQMVVGC |
| 24529 | DIVIKIGMG |
| 24530 | DKGCKCQWG |
| 24531 | DKKPVEDDM |
| 24532 | DKVVQQCKD |
| 24533 | DLEHKHQFF |
| 24534 | DLRCDPQMW |
| 24535 | DMDAQGAMW |
| 24536 | DMYFTRHTW |
| 24537 | DMYSMVKHN |
| 24538 | DNFPWCTGN |
| 24539 | DQCVLSRGH |
| 24540 | DQEWSVHMG |
| 24541 | DQFCTYDYT |
| 24542 | DQGPSESDL |
| 24543 | DQKVMFECY |
| 24544 | DQQCAQMGV |
| 24545 | DRQCCKLTF |
| 24546 | DSHRLPGNW |
| 24547 | DSMHYYMMH |
| 24548 | DSNYEEKVI |
| 24549 | DSQYMCVHR |
| 24550 | DSRQFENQC |
| 24551 | DSTGRPMMG |
| 24552 | DSVAHSSQK |
| 24553 | DTDRGLVYE |
| 24554 | DTHAVEFAA |
| 24555 | DTHWWNILC |
| 24556 | DTYGIQDFQ |
| 24557 | DVDFEWSQW |
| 24558 | DVEPEYCAG |
| 24559 | DVFIDWSGQ |
| 24560 | DVIMGPIAN |
| 24561 | DVINRYECG |
| 24562 | DVKQQPLAI |
| 24563 | DVKTDTSPD |
| 24564 | DVLLADTEP |
| 24565 | DVRDNEPER |
| 24566 | DVRQILMCH |
| 24567 | DVWQNSGAL |
| 24568 | DWNQVDNYY |
| 24569 | DWPAQKGWA |
| 24570 | DWQPMLHMN |
| 24571 | DYLAMNLHF |
| 24572 | DYMHMLTAQ |
| 24573 | DYMQNQYGT |
| 24574 | EAEIFRLEG |
| 24575 | EALMPMLGA |
| 24576 | EASTQWLMQ |
| 24577 | EATCDPPYN |
| 24578 | EATIMDQVV |
| 24579 | EATKQSWTH |
| 24580 | EAYLTIDKL |
| 24581 | ECDRYIAEY |
| 24582 | ECGLWWGSE |
| 24583 | ECKPIAGDK |
| 24584 | ECPNVNTAQ |
| 24585 | ECVANPFQP |
| 24586 | ECYHVLANS |
| 24587 | EDTHMIAGH |
| 24588 | EEFDTVAAP |
| 24589 | EFMLSSKDI |
| 24590 | EFYHFMYKD |
| 24591 | EGVESMLYA |
| 24592 | EHDWITVYE |
| 24593 | EHVIIKKNY |
| 24594 | EICVVTGEQ |
| 24595 | EIEMMGRGE |
| 24596 | EIKVVNSRD |
| 24597 | EIKYRRCMA |
| 24598 | EIRECEECC |
| 24599 | EITDWGVVP |
| 24600 | EITYRRCMA |
| 24601 | EIYMRHNFP |
| 24602 | EKAHSWQDP |
| 24603 | EKISICSQY |
| 24604 | EKMESPECI |
| 24605 | EKPYHEEKF |
| 24606 | ELLFMNAMS |
| 24607 | EMARNIEQN |
| 24608 | EMRMNIQWE |
| 24609 | ENCEPMHCE |
| 24610 | ENCHITKMD |
| 24611 | ENSYTHDTI |
| 24612 | ENTLFMRYH |
| 24613 | EPKQEEWYV |
| 24614 | EPVTFVGQA |
| 24615 | EPYFCCCVP |
| 24616 | ERDHVNISC |
| 24617 | ERSWYYDQY |
| 24618 | ESCEGILTF |
| 24619 | ESDPMQQFT |
| 24620 | ESEYNSSYN |
| 24621 | ESKSCGLTG |
| 24622 | ESNLTPWSQ |
| 24623 | ESRWMDEHT |
| 24624 | ESTHKFMQD |
| 24625 | ESYSTAEMA |
| 24626 | ETEWAWVTQ |
| 24627 | ETLPIEIRS |
| 24628 | ETSKWHAVC |
| 24629 | ETVLHQWAA |
| 24630 | EVAMLEEDK |
| 24631 | EVCLRHYWQ |
| 24632 | EVDCKVSHV |
| 24633 | EVDVHDEWV |
| 24634 | EVEAIRGIQ |
| 24635 | EVEFMSYRW |
| 24636 | EVISYVENV |
| 24637 | EVLHRDLHM |
| 24638 | EVMCSDVPK |
| 24639 | EVNCTHLYN |
| 24640 | EYARKTKGW |
| 24641 | EYPYRYEFS |
| 24642 | EYRWMPNHL |
| 24643 | FACQCKGCS |
| 24644 | FAKYMNHGG |
| 24645 | FDEPMIHQW |
| 24646 | FEYIWVYWY |
| 24647 | FFCPKHKWP |
| 24648 | FICLRYKDQ |
| 24649 | FIWVCHTYK |
| 24650 | FKMWKLFQA |
| 24651 | FLELMQIGL |
| 24652 | FLRYMVCAH |
| 24653 | FMEQDIGHA |
| 24654 | FNGSICDTY |
| 24655 | FNNNDVHPI |
| 24656 | FPSAYQHQH |
| 24657 | FQSMGEIWC |
| 24658 | FRGHGMWDA |
| 24659 | FRLCSDVRC |
| 24660 | FSNLVEVSK |
| 24661 | FSTCTSCCA |
| 24662 | FTCPYKFGM |
| 24663 | FTEQIDRQM |
| 24664 | FTMASAQAD |
| 24665 | FVCLGRKGP |
| 24666 | FVGWYWNSP |
| 24667 | FWKTKSGGA |
| 24668 | FWQMKSCLP |
| 24669 | FWVLDMCQA |
| 24670 | FWYPKDRMV |
| 24671 | GACERRDAM |
| 24672 | GADQCMLWE |
| 24673 | GAIYFNKLG |
| 24674 | GCAPLVQDD |
| 24675 | GCAYQAVNP |
| 24676 | GCISTLNGM |
| 24677 | GCMKLVGTA |
| 24678 | GEVVPHISW |
| 24679 | GFHLEEKYD |
| 24680 | GFKQYATYI |
| 24681 | GGHFDGWQR |
| 24682 | GGMWLQNVS |
| 24683 | GGWFMGVMG |
| 24684 | GHREFQMPY |
| 24685 | GHVAYPPDQ |
| 24686 | GIARMFRWV |
| 24687 | GIGYKWFPG |
| 24688 | GLMPVMNYT |
| 24689 | GNATKIKLN |
| 24690 | GNRRCWVPF |
| 24691 | GQGGNCKSH |
| 24692 | GSHEYWEMA |
| 24693 | GSLFQNYQW |
| 24694 | GSYMACRMD |
| 24695 | GTCNFQMDS |
| 24696 | GTDCFWWHE |
| 24697 | GTSFVGNPL |
| 24698 | GTYVCQNHD |
| 24699 | GVAHPEAVC |
| 24700 | GVTEHNEVC |
| 24701 | GVVDESYDP |
| 24702 | GVVGLNTCV |
| 24703 | GWCNRYFWD |
| 24704 | GWIRYKNSE |
| 24705 | GYFGKCKRI |
| 24706 | HADMEQGNS |
| 24707 | HADSNIACN |
| 24708 | HAESMVCHH |
| 24709 | HASLNLNPS |
| 24710 | HCIWNWSDA |
| 24711 | HEAKQVPKA |
| 24712 | HERCSELEY |
| 24713 | HEYIYETWP |
| 24714 | HFYVGSPED |
| 24715 | HHQYIDCFV |
| 24716 | HIDPDHWHG |
| 24717 | HKLYEDRIN |
| 24718 | HKNDLAAVQ |
| 24719 | HKSDMWWTA |
| 24720 | HLPAPELYL |
| 24721 | HMDIQMFMP |
| 24722 | HNFCMYWPQ |
| 24723 | HNHSADSFA |
| 24724 | HPELLDRFE |
| 24725 | HPSSTWSIL |
| 24726 | HQNKFEWSP |
| 24727 | HRNEQLIRA |
| 24728 | HSGELYDGF |
| 24729 | HSMLETTGK |
| 24730 | HSNFFHMND |
| 24731 | HSYQQCAVG |
| 24732 | HTPQVLKGE |
| 24733 | HTTAHQNWA |
| 24734 | HTTFALVMH |
| 24735 | HVDSNIACN |
| 24736 | HVKSPTFQT |
| 24737 | HVKVHEMMI |
| 24738 | HVMPKQGFN |
| 24739 | HYCKIIPYH |
| 24740 | HYEYCFGIF |
| 24741 | IAGTYTNWY |
| 24742 | IAMDNQNTD |
| 24743 | IAQDTGTLH |
| 24744 | ICKIGSRLD |
| 24745 | IEGHWAQFP |
| 24746 | IEIATVNVH |
| 24747 | IFMVHDSDY |
| 24748 | IFVGKNAAW |
| 24749 | IGIQFLKDH |
| 24750 | IGTYGWIEA |
| 24751 | IHDASTKDQ |
| 24752 | IHMHEVGVY |
| 24753 | IIKDVGDFG |
| 24754 | IIPQVDQYY |
| 24755 | IIRDKTQDD |
| 24756 | IKCDKCQDQ |
| 24757 | IKGRFDWDC |
| 24758 | IMEYVHKPG |
| 24759 | IQAATNLWQ |
| 24760 | IQGEVTVTA |
| 24761 | IQGPEAIMK |
| 24762 | IQGVQWMNH |
| 24763 | IREEMHEQF |
| 24764 | ISGLAQMGM |
| 24765 | ISICYHMFP |
| 24766 | ISMQICAGW |
| 24767 | ITCQSNEQH |
| 24768 | ITGTRFAEV |
| 24769 | ITMPAEAFQ |
| 24770 | ITTKYQIDH |
| 24771 | ITTPQLNSV |
| 24772 | IVDHLPRHG |
| 24773 | IVGLATTAM |
| 24774 | IVHPTLNTP |
| 24775 | IVMREHGAC |
| 24776 | IWADLECGR |
| 24777 | IWDLAGDAM |
| 24778 | IYNQRQMCP |
| 24779 | KACDRQIYE |
| 24780 | KACEMYASF |
| 24781 | KADAKVPCC |
| 24782 | KADVRSAQH |
| 24783 | KASNDYLCR |
| 24784 | KCDGLWCHF |
| 24785 | KDRMGLNQC |
| 24786 | KDVTKLRCP |
| 24787 | KEPITAEHK |
| 24788 | KFAGAGIGW |
| 24789 | KGKLEYRIR |
| 24790 | KGLDVPHWG |
| 24791 | KHCEILCNW |
| 24792 | KIAEWEDWM |
| 24793 | KICSQLDSP |
| 24794 | KKFWDTVIC |
| 24795 | KKNCREPEQ |
| 24796 | KMQAMGVNW |
| 24797 | KPTKYEWLH |
| 24798 | KREAQMVGN |
| 24799 | KSLIQQSWD |
| 24800 | KSMVIRHAQ |
| 24801 | KTCHPEGMP |
| 24802 | KTNMHAKSV |
| 24803 | KTNRTYMWR |
| 24804 | KVEACQSVE |
| 24805 | KVGAHVAQE |
| 24806 | KVGIHDSLV |
| 24807 | KVKWKDKDL |
| 24808 | KVYYIISHQ |
| 24809 | KWIMWIEYN |
| 24810 | KYHNVPSKI |
| 24811 | KYQYYQEGP |
| 24812 | LAGCLCYTN |
| 24813 | LAGCLWYTN |
| 24814 | LAICHSNLA |
| 24815 | LANMPNMGF |
| 24816 | LATQACSCN |
| 24817 | LCASRGLDP |
| 24818 | LCGPIIKAS |
| 24819 | LCIRLWTSA |
| 24820 | LCPNWWRYQ |
| 24821 | LDRKACMIA |
| 24822 | LEAELMGHC |
| 24823 | LEVDMHASY |
| 24824 | LFEQKMVWS |
| 24825 | LFSPYEACC |
| 24826 | LFVVTTTNN |
| 24827 | LGSLYRRRG |
| 24828 | LGYHVQEAG |
| 24829 | LHCQMGDHW |
| 24830 | LHEIQKPNH |
| 24831 | LHSQADGLY |
| 24832 | LHTPQHDCH |
| 24833 | LKDQFHWGA |
| 24834 | LKELIQFGN |
| 24835 | LKMDMTKRS |
| 24836 | LLPPMMMGQ |
| 24837 | LNLYWFWSA |
| 24838 | LNPMTQNHE |
| 24839 | LPLQVSNMA |
| 24840 | LPRLNLVDR |
| 24841 | LQDVIVDCE |
| 24842 | LQTPQHHGA |
| 24843 | LRYYIEMDR |
| 24844 | LSQMLAHTA |
| 24845 | LSVWAIRND |
| 24846 | LTHKRESEC |
| 24847 | LTMGFEIGW |
| 24848 | LTNPWHYFA |
| 24849 | LTVIIFDDA |
| 24850 | LVAMVPQSY |
| 24851 | LVDQGKCMQ |
| 24852 | LWHWLWLKT |
| 24853 | LWTRMVYFG |
| 24854 | LYAHGNCYP |
| 24855 | LYASVMQSE |
| 24856 | LYKMVTKLH |
| 24857 | MAPTVVRKK |
| 24858 | MATPKEVMQ |
| 24859 | MCAGWCDDQ |
| 24860 | MCGLRYYWP |
| 24861 | MCLWIRACE |
| 24862 | MCPNHKPCW |
| 24863 | MESLCVYFY |
| 24864 | MFMKNELIA |
| 24865 | MGCQSQKPK |
| 24866 | MGMFNTMAE |
| 24867 | MHETHTWGA |
| 24868 | MHMSTLDAK |
| 24869 | MHVQQQRYG |
| 24870 | MIYKDYNHA |
| 24871 | MIYPCMTSC |
| 24872 | MKHESDGGF |
| 24873 | MLAGWWQET |
| 24874 | MMIVQCSNK |
| 24875 | MMKWTEYMV |
| 24876 | MMTLKNTMD |
| 24877 | MNFNSLAQW |
| 24878 | MNLPKSHDC |
| 24879 | MNMYYTGEV |
| 24880 | MNNIYECIP |
| 24881 | MNSHNHTST |
| 24882 | MNTPTFHQI |
| 24883 | MPTMMGSYM |
| 24884 | MQFRTNNAN |
| 24885 | MQNISCPQC |
| 24886 | MQPRYNCEG |
| 24887 | MQSFVGVMW |
| 24888 | MRALSVSQG |
| 24889 | MRTMFYTSP |
| 24890 | MRVIQGDNY |
| 24891 | MSFTYVGGS |
| 24892 | MSTKPAYWG |
| 24893 | MSTKPVYWG |
| 24894 | MTDLQLIER |
| 24895 | MTEILQITG |
| 24896 | MTEPFMQDL |
| 24897 | MTIGYFVQA |
| 24898 | MTNRMQALQ |
| 24899 | MTRFKIYHE |
| 24900 | MTTHCNTAI |
| 24901 | MVNCAVTYQ |
| 24902 | MVSDTQHGK |
| 24903 | MVSYDVLGK |
| 24904 | MVSYNVLGK |
| 24905 | MVTTQQIVC |
| 24906 | MVVIKQYHT |
| 24907 | MVVVTRDAS |
| 24908 | MWGEWWPGC |
| 24909 | MWQEFQWQY |
| 24910 | MYIPRNGEP |
| 24911 | NACMVTGCG |
| 24912 | NAEWQRDTF |
| 24913 | NASPCQIFG |
| 24914 | NCFVMYCGR |
| 24915 | NDKWGWWRE |
| 24916 | NDVMWQLIH |
| 24917 | NFDFNHQCQ |
| 24918 | NFEIYHLQC |
| 24919 | NFGIYHLQC |
| 24920 | NFNWTRADC |
| 24921 | NFRTINAHP |
| 24922 | NGDPVFRMA |
| 24923 | NIEGIEFNA |
| 24924 | NIVGATEWD |
| 24925 | NKDDTCFQF |
| 24926 | NKYCIAMPC |
| 24927 | NLGETITNA |
| 24928 | NMIHLWTTE |
| 24929 | NMNQNPTMS |
| 24930 | NMYWCMQIH |
| 24931 | NNRETMIHG |
| 24932 | NNREWDVCY |
| 24933 | NPPDVGCPS |
| 24934 | NPQSLCYSC |
| 24935 | NQHWLLSVP |
| 24936 | NQMPCWVFF |
| 24937 | NSCFIAENC |
| 24938 | NSGIATADH |
| 24939 | NSIQYGQAP |
| 24940 | NSKSPSEGC |
| 24941 | NSQPWYRHS |
| 24942 | NSTAHAFAY |
| 24943 | NSTAHPMMW |
| 24944 | NTHVCNLHE |
| 24945 | NTLFWDGNP |
| 24946 | NVEERNRHF |
| 24947 | NVEKQVAMG |
| 24948 | NVHHTTQIP |
| 24949 | NVMSALDLS |
| 24950 | NVTQWDEDL |
| 24951 | NVVQCNGPH |
| 24952 | NWFGINSWH |
| 24953 | PANYSMPNC |
| 24954 | PDGYRRSHG |
| 24955 | PDWCQDPGM |
| 24956 | PEELYLYRK |
| 24957 | PEYRQWMLI |
| 24958 | PFLFTVIHN |
| 24959 | PFVHDDMAR |
| 24960 | PGEENEPPA |
| 24961 | PIWTQHHTM |
| 24962 | PLQQYDGCS |
| 24963 | PQHCNTMWG |
| 24964 | PQIDSLQDY |
| 24965 | PRNDDNCLP |
| 24966 | PVIWLPVDS |
| 24967 | PWDKEEPQG |
| 24968 | PYAIEESHD |
| 24969 | PYIRRVHVS |
| 24970 | QAACAMATT |
| 24971 | QADKQFYGN |
| 24972 | QAGYQSALY |
| 24973 | QANGENLDH |
| 24974 | QCDRVWLNP |
| 24975 | QCIPKQKDY |
| 24976 | QCQTLDCCD |
| 24977 | QCWYCELCP |
| 24978 | QESEVHVFG |
| 24979 | QFQLVTIQG |
| 24980 | QGAFHTAWT |
| 24981 | QGEKCVPDF |
| 24982 | QGIMTSCQT |
| 24983 | QGVRMVGNS |
| 24984 | QHCMHSMVE |
| 24985 | QHQHDLPTG |
| 24986 | QIAVVCSQR |
| 24987 | QIAVVCSQW |
| 24988 | QIFKALVGE |
| 24989 | QIGWDDAIT |
| 24990 | QIISETGWE |
| 24991 | QKFCNIHEY |
| 24992 | QKQTSISFS |
| 24993 | QKTWLVVWC |
| 24994 | QMATPVDMT |
| 24995 | QMGCTFQTP |
| 24996 | QMGLLDTYP |
| 24997 | QMYMDFTQP |
| 24998 | QNYWCMCGA |
| 24999 | QPKEWVIKE |
| 25000 | QPRNDCNEH |
| 25001 | QQAMYTSSG |
| 25002 | QQDPMKQWS |
| 25003 | QQEVEQTMG |
| 25004 | QQEYFDDMQ |
| 25005 | QQSAVGAHY |
| 25006 | QQVYEMKWH |
| 25007 | QRVMSGKPL |
| 25008 | QSDPQPVIW |
| 25009 | QSGIHCCCG |
| 25010 | QSGWFLEAK |
| 25011 | QSHFCFENA |
| 25012 | QSIPEWSCA |
| 25013 | QSNGQKDSQ |
| 25014 | QSVDWIKCE |
| 25015 | QTDQYWKMG |
| 25016 | QTGWLHSYG |
| 25017 | QTKTGEIQK |
| 25018 | QTKTGKSAT |
| 25019 | QVHTKWDGM |
| 25020 | QVQYRTCDW |
| 25021 | QWTEKTFEG |
| 25022 | QYEPLGRAM |
| 25023 | QYKSTQRRN |
| 25024 | QYYMLSESA |
| 25025 | RAKTCIYKC |
| 25026 | RAMCILHNQ |
| 25027 | RAMPTAAQL |
| 25028 | RDLPIQFGP |
| 25029 | RDMDMQMWC |
| 25030 | REAKVFELK |
| 25031 | REGCYNTQE |
| 25032 | RFELLMTGR |
| 25033 | RGFWVEKSG |
| 25034 | RGSAKDAWG |
| 25035 | RISSEGMWC |
| 25036 | RLHIDDEDH |
| 25037 | RLVRDSKNN |
| 25038 | RMEELINDK |
| 25039 | RMVTWDQSE |
| 25040 | RNMCRHNDI |
| 25041 | RPETVHASF |
| 25042 | RQGCLLAWP |
| 25043 | RQICNEPWN |
| 25044 | RRNELLIPD |
| 25045 | RSEMVNTQM |
| 25046 | RSQWHNSID |
| 25047 | RSSEYLTQQ |
| 25048 | RTLVKGAQC |
| 25049 | RTQGCGPSR |
| 25050 | RVADVMYIG |
| 25051 | RVECRPWGE |
| 25052 | RVMDNACDM |
| 25053 | RWDYYWTGM |
| 25054 | RWRNCGSNG |
| 25055 | SACIFYAHK |
| 25056 | SAEMWVKDE |
| 25057 | SALPCNTAQ |
| 25058 | SARTEERES |
| 25059 | SATTMPRTD |
| 25060 | SCFINSAYP |
| 25061 | SCGLVQRFM |
| 25062 | SCGQVEHYC |
| 25063 | SCQMYTYGI |
| 25064 | SCRLRFNYA |
| 25065 | SCTTCHSWQ |
| 25066 | SFASMYNIK |
| 25067 | SFMYKTQAL |
| 25068 | SFYKNGMSN |
| 25069 | SGGFCNYAT |
| 25070 | SIGPASNAL |
| 25071 | SIHMAEGVE |
| 25072 | SIHRWNWMG |
| 25073 | SINPESQGS |
| 25074 | SISPSMGEK |
| 25075 | SIVHPPTTS |
| 25076 | SKGCIIDMQ |
| 25077 | SKLLVTRNH |
| 25078 | SKRLVTRNH |
| 25079 | SLKDVDLWH |
| 25080 | SLYKHEIWW |
| 25081 | SMFPFNMGP |
| 25082 | SMTDCDFHQ |
| 25083 | SNHPNMHTY |
| 25084 | SQGNTRYSY |
| 25085 | SRCLRHQEC |
| 25086 | SRMDMNHGL |
| 25087 | SRTMYKWMP |
| 25088 | SRTNIEWGG |
| 25089 | SRYKCQHRN |
| 25090 | SSDQLIMWG |
| 25091 | SSHATNFCG |
| 25092 | SSHTTNFCG |
| 25093 | SSTGMQSST |
| 25094 | SSTTHSRMQ |
| 25095 | STFPKWTSE |
| 25096 | STHFRTEDR |
| 25097 | STLVYPCGP |
| 25098 | STSAQPNFD |
| 25099 | STTQWGMCN |
| 25100 | STVSQMNYF |
| 25101 | SVCPSHMWM |
| 25102 | SVFSFHSDT |
| 25103 | SWDVRVTAQ |
| 25104 | SWSMQNKST |
| 25105 | SWVSADEVP |
| 25106 | SYGQIECYQ |
| 25107 | SYLRAHHKD |
| 25108 | TAALMGNMA |
| 25109 | TALDNSTGI |
| 25110 | TAQLMQTAC |
| 25111 | TAYLNNWVP |
| 25112 | TAYPEYCGN |
| 25113 | TCAPQGKQE |
| 25114 | TCAVFVFHC |
| 25115 | TCEEYYNPF |
| 25116 | TCELMQVMH |
| 25117 | TCQNIAMQL |

TABLE 80
Sequences of the 581 to 589 Region in AAV5
VP1 Capsid Polypeptide that Drive Sciatic
Nerve Tissue Tropism

| SEQ ID | 581-589 |
|---|---|
| NO | Sequence |
| 27991 | EIILDDQQR |
| 27992 | LDDNAWRHR |
| 27993 | LGGKPYALD |
| 27994 | GSQQTTLSC |
| 27995 | TWESQIDCI |
| 27996 | VVGLKPMHM |
| 27997 | NFNINIYNC |
| 27998 | ANHATGGQS |
| 27999 | EFKRMTCNK |
| 28000 | NKHQGMHRM |
| 28001 | KGGPWAYNC |
| 28002 | VLIPIGQHY |
| 28003 | DPFECTVWP |
| 28004 | TVQWNFAHM |
| 28005 | TAYMTDMYC |
| 28006 | SATNWECSN |
| 28007 | KVDAEGFHF |
| 28008 | VWEDNPPFR |
| 28009 | SVLMSPHLM |
| 28010 | LEKNSSQMR |
| 28011 | CRCNDAWNM |
| 28012 | AAPFMWRDL |
| 28013 | ACKYPPHVR |
| 28014 | ANKMFMEQA |
| 28015 | ANKMFMGQA |
| 28016 | ANKMVMEQA |
| 28017 | AWSEFQWFD |
| 28018 | CYRCLPPEA |
| 28019 | DIDRHAHFD |
| 28020 | DKGYSLYQP |
| 28021 | DRDWSDVRR |
| 28022 | DRGWSDVRR |
| 28023 | EHGYMRCTK |
| 28024 | EHGYMRCTR |
| 28025 | EHGYMRCTT |
| 28026 | EHGYMRYTK |
| 28027 | EIECLQTVA |
| 28028 | EIEFLQTVA |
| 28029 | EIEFLQTVS |
| 28030 | EIEFLQTVV |
| 28031 | EIEILQTVA |
| 28032 | EIEVLQTVA |
| 28033 | EIFLDDQQR |
| 28034 | EIILDAQQR |
| 28035 | EIILDDEQR |
| 28036 | EIILDDQQC |
| 28037 | EIILDDQQH |
| 28038 | EIILDDQRR |
| 28039 | EIILDGQQR |
| 28040 | EIILDVQQR |
| 28041 | EIILGDQQR |
| 28042 | ELALGVLWK |
| 28043 | FPPFCALCT |
| 28044 | FPPFYALCT |
| 28045 | FPPFYAPCT |
| 28046 | FPPFYTLCT |
| 28047 | FTAYPQAVM |
| 28048 | GAIVFDLCD |
| 28049 | GAWDYMYEC |
| 28050 | GCFWAANSC |
| 28051 | GCFWAANSG |
| 28052 | GCFWATNSG |
| 28053 | GIILDDQQR |
| 28054 | GMPTNHQYI |
| 28055 | GMPTNRQYI |
| 28056 | GMSTNRQYI |
| 28057 | GNLIDWQHI |
| 28058 | GNLIDWRHI |
| 28059 | HADFERHSQ |
| 28060 | HADFGRHSQ |
| 28061 | HLSFMDLGS |
| 28062 | HLSFMNLCS |
| 28063 | HLSFMNLDS |
| 28064 | HLSFMNLGI |
| 28065 | HLSFMNLGS |
| 28066 | HLSFMSLGS |
| 28067 | HLSFVNLGS |
| 28068 | IALTVTRFP |
| 28069 | IQHWMSHSH |
| 28070 | IQHWMSHSR |
| 28071 | ISHCHFDHK |
| 28072 | ISHSHFDHK |
| 28073 | ISHYHFDHE |
| 28074 | ISHYHFDHK |
| 28075 | ISHYHFDHR |
| 28076 | ISHYHFDQK |
| 28077 | ISHYHFGHK |
| 28078 | ITQCTMMGV |
| 28079 | KLPGSHAER |
| 28080 | KLPGSHTER |
| 28081 | KLPGSRAER |
| 28082 | KYASVLEQA |
| 28083 | KYASVPEQA |
| 28084 | LDLKWWCTF |
| 28085 | MFFQKPKWS |
| 28086 | MIALRMEGN |
| 28087 | MIDLRMEGN |
| 28088 | MIDLRMEGT |
| 28089 | MIDLRMGGN |
| 28090 | MIDLRVEGN |
| 28091 | MIDLWMEGN |
| 28092 | MQEQQKAYE |
| 28093 | MQEQQKTYA |
| 28094 | MQEQQKTYE |
| 28095 | MQEQQRTYE |
| 28096 | MQQSEQCYP |
| 28097 | MQQSEQCYQ |
| 28098 | MQQSEQFYQ |
| 28099 | MQQSGQCYQ |
| 28100 | MREQQKTYE |
| 28101 | MSFQEPKWS |
| 28102 | MSFQKPKWS |
| 28103 | MSFQKPMWS |
| 28104 | MSFQKPRWS |
| 28105 | MSFQKTKWS |
| 28106 | NCLPWGQFC |
| 28107 | NCLPWSQFC |
| 28108 | NCLSWSQFC |
| 28109 | NLRECTIMN |
| 28110 | NLREGTIMN |
| 28111 | NLWECTIMN |
| 28112 | NRGCSDRIS |
| 28113 | NWLPWSQFC |
| 28114 | NYIEACAMN |
| 28115 | PREICWSHS |
| 28116 | PVCSTHFWQ |
| 28117 | PVSMGTKRE |
| 28118 | PVSMGTRRE |
| 28119 | QCRHEGSNG |
| 28120 | QCRHEWSNG |
| 28121 | QCYTENDTK |
| 28122 | QHNYVSVVE |
| 28123 | QHNYVSVVG |
| 28124 | QHVGLQAEK |
| 28125 | RENTFDQIR |
| 28126 | RHGHGHHYD |
| 28127 | RHSHGHHYD |
| 28128 | RHSHGHHYG |
| 28129 | RHSHGHHYY |
| 28130 | RHSHGPHYD |
| 28131 | RLSFMNLGS |
| 28132 | SIEVRPPLC |
| 28133 | SIEVWPPLC |
| 28134 | SKAKYKTQM |
| 28135 | SMEFEDQCV |
| 28136 | SMEFGDQCV |
| 28137 | SMEVEDQCV |
| 28138 | SVEVRPPLC |
| 28139 | TFCFVQTLV |
| 28140 | TFCFVQTRV |
| 28141 | TFKFNWMDN |
| 28142 | TFKFNWMDS |
| 28143 | TFKFNWMDT |
| 28144 | TFKFNWMGN |
| 28145 | TFKFNWMYN |
| 28146 | TFKVNWMDN |
| 28147 | TFRFNWMDN |
| 28148 | TFTFNWMDN |
| 28149 | THCPFQHDI |
| 28150 | THCQFPHDI |
| 28151 | THCQFQHDI |
| 28152 | THCQFQHDM |
| 28153 | TNKMFMEQA |
| 28154 | VAIVFDLCD |
| 28155 | VAIVFGLCD |
| 28156 | VAWDCMYEC |
| 28157 | VAWDDMYEC |
| 28158 | VAWDYMYEC |
| 28159 | VAWDYMYEG |
| 28160 | VAWGYMYEC |
| 28161 | WANLRQMDL |
| 28162 | WGNLRQMDL |
| 28163 | WSSREYLHK |
| 28164 | WVNLRQMDL |
| 28165 | WVNLRQMGL |
| 28166 | WVNLWQMDL |
| 28167 | YYYSSKSSV |
| 28168 | YYYSSTSSV |
| 28169 | WSSREYWHR |
| 28170 | WSSRGYWHK |
| 28171 | WSSREDWHK |
| 28172 | MHNPLWFSS |
| 28173 | ACVDHMFER |
| 28174 | AGYTIHTVA |
| 28175 | AGYTIHTVE |
| 28176 | AIHLPSACW |
| 28177 | AITMDAQPN |
| 28178 | ATIDHNSPG |
| 28179 | AVGSEDSSD |
| 28180 | CAHIMDELG |
| 28181 | CDINVLIMM |
| 28182 | CDINVLIMV |
| 28183 | CDKLDKPAV |
| 28184 | CDKLDKPTV |
| 28185 | CFAHMQIAN |
| 28186 | CISNHNSTY |
| 28187 | CLSMFVGNT |
| 28188 | CVKLYVNED |
| 28189 | CVKLYVNGD |
| 28190 | CVRKCDLCG |
| 28191 | DMPWVDYMG |
| 28192 | DMTFEAYPT |
| 28193 | DMTFEPYPT |
| 28194 | DMTFETYPT |
| 28195 | DNDVEMAAK |
| 28196 | DSDNGPNIF |
| 28197 | EAHPHMMSQ |
| 28198 | EAQPHMMSQ |
| 28199 | ECGRYGVFS |
| 28200 | EFMEVMDQV |
| 28201 | EGGFQDFCE |
| 28202 | EGGFRDFWE |
| 28203 | EMLRCCDCT |
| 28204 | EQLDVQEVY |
| 28205 | EVDVSNDNC |
| 28206 | EVDVSNNNC |
| 28207 | EVSDGTPTV |
| 28208 | FALWSNCAE |
| 28209 | FALWSNCGE |
| 28210 | FALWSNCVE |
| 28211 | FDCHNASKP |
| 28212 | FLTEMQWHA |
| 28213 | FLTGMQWHA |
| 28214 | FVADWIWPA |
| 28215 | FVADWIWPT |
| 28216 | FVADWIWSA |
| 28217 | GCFTFWDHQ |
| 28218 | GCLQVNQME |
| 28219 | GCNMIPKHD |
| 28220 | GDMPERETG |
| 28221 | GHVSTSNQG |
| 28222 | GQHSLVTMY |
| 28223 | GVQFRYSMP |
| 28224 | GVRVQDSHH |
| 28225 | GVTHKAKHE |
| 28226 | GVWVQDSHH |
| 28227 | HSDYEMNDD |
| 28228 | HYTEGYRQT |
| 28229 | IADHFLAHF |
| 28230 | IADLCNCEN |
| 28231 | IGAPVDTIN |
| 28232 | IGTAFRNSG |
| 28233 | KCESDYLPE |
| 28234 | KTNWWDGQC |
| 28235 | LAIRKIANH |
| 28236 | LAIRKITNH |
| 28237 | LARYCEKTP |
| 28238 | LCMESIMMI |
| 28239 | LCMESLMMI |
| 28240 | LCTESLMMI |
| 28241 | LDRRSSAYS |
| 28242 | LFSAWNDFA |
| 28243 | LFSAWNDFV |
| 28244 | LMCYMQKIT |
| 28245 | LMGYMQKIT |
| 28246 | LMSTMFNTF |
| 28247 | LQVHWWLLL |
| 28248 | LTIPLHAWS |
| 28249 | LYGQVVAID |
| 28250 | MAIFDSAAV |
| 28251 | MFWSGNKQG |
| 28252 | MQCDFLHDT |
| 28253 | MQKDVEQMW |
| 28254 | MSMCYGIKG |
| 28255 | NAMTQGNHQ |
| 28256 | NAMTQINHQ |
| 28257 | NAMTQVNHQ |
| 28258 | NAMTQVNHR |
| 28259 | NHNPTNRNC |
| 28260 | NKPCKYMYK |
| 28261 | NPTTVCLKM |
| 28262 | NTHEGTDGN |
| 28263 | NTTPNCNSW |
| 28264 | NTWPYPENS |
| 28265 | NYLQFGHKQ |
| 28266 | PGTQPYMGW |
| 28267 | PQEIKSTSC |
| 28268 | PRECHQSHQ |
| 28269 | QASFKQMGD |
| 28270 | QASFKQMGG |
| 28271 | QATCYDTSP |
| 28272 | QFTHNQAAN |
| 28273 | QGIQIGKCQ |
| 28274 | QHVAFNRWH |
| 28275 | QILDQQEPP |
| 28276 | QINNVVDNR |
| 28277 | QISMNVAFR |
| 28278 | QMAEFTYYG |
| 28279 | QMFDTWKCW |
| 28280 | QTSSYDAHC |
| 28281 | QTSSYDTHC |
| 28282 | QVTGIPWFY |
| 28283 | QWEDRTQMS |
| 28284 | RATCYDTSP |
| 28285 | REMEYQGER |
| 28286 | RPPNRVATG |
| 28287 | SAMPETTIF |
| 28288 | SAMPGTTIF |
| 28289 | SQPMLKVNA |
| 28290 | SSSDLCQNR |
| 28291 | TCMPFGTSQ |
| 28292 | TFKLTTGGE |
| 28293 | THDDCQWWE |
| 28294 | TIHLPSACW |
| 28295 | TIKLTTGGE |
| 28296 | VIMWNYEHY |
| 28297 | VSVSIYSGW |
| 28298 | VTMVKRKGT |
| 28299 | YDDWYSNVY |
| 28300 | YPMPMCCQP |
| 28301 | YPMPMCCQS |
| 28302 | WCRTPQREN |
| 28303 | GMTNEVQAA |
| 28304 | EGGFRDFCE |
| 28305 | RYEISERRC |
| 28306 | SAISCQCAK |
| 28307 | EVQRNIYSP |
| 28308 | HCTEGYRQT |
| 28309 | SAPEFSVIC |
| 28310 | MFRQFYIQG |
| 28311 | STVGQNHCG |
| 28312 | EFCHQHSAI |
| 28313 | TGWNFPVSC |
| 28314 | GCLTSECAY |
| 28315 | TMFYCHGPN |
| 28316 | HITPMITMW |
| 28317 | AITRHVADT |
| 28318 | QRSVFHQRS |
| 28319 | VKRLENEWH |
| 28320 | MMAMKQGFY |
| 28321 | EGRTISYGN |
| 28322 | STCYPFAMG |
| 28323 | QIIYRCRST |
| 28324 | GTAVSRQSK |
| 28325 | GCEEVGRCQ |
| 28326 | QHCFRAMED |
| 28327 | MNSFYRAEW |
| 28328 | VCHNPDPFT |
| 28329 | NGYLVHACQ |
| 28330 | NPGWQTIGQ |
| 28331 | LCAVCGDCD |
| 28332 | ATLMSDGGG |
| 28333 | YFPSIQCYE |
| 28334 | GAANRIQSW |
| 28335 | MFMTNVNSF |
| 28336 | VSVQSRAQL |
| 28337 | PLTIEVNCT |
| 28338 | KTMGIGGGP |
| 28339 | NRIQMTDFQ |
| 28340 | DFNGSNWLP |
| 28341 | FAIWKRNES |
| 28342 | HFQPVFMQP |
| 28343 | DQEAQVVTG |
| 28344 | SECEVLCSL |
| 28345 | AAMESCAEI |
| 28346 | NQREWHGLA |
| 28347 | NYVNQGMGP |
| 28348 | NISPYEMAV |
| 28349 | ATGTYNLQE |
| 28350 | HATGLVNFM |
| 28351 | NAIPYFVQA |
| 28352 | CVEQYVEQG |
| 28353 | AVAEIRPEP |
| 28354 | ASKQGTHDY |
| 28355 | SSEYSQFAI |
| 28356 | TMTVRMEQP |
| 28357 | MWGCKLFVC |
| 28358 | AGMYKCQED |
| 28359 | TYESLQNSW |
| 28360 | TKMIIFDSW |
| 28361 | LCKFANGAA |
| 28362 | INYISFVDR |
| 28363 | RQAWPKDGM |
| 28364 | QSAHPEPMC |
| 28365 | YCECGHWPN |
| 28366 | SQERMDYDG |
| 28367 | WINEANCLM |
| 28368 | SNVACIQAF |
| 28369 | YTKYNWKAA |
| 28370 | NYAMRRDSY |
| 28371 | PVICVWHDN |
| 28372 | SFKSVSQYN |
| 28373 | ENTMWQSSQ |
| 28374 | NAYYDSHCE |
| 28375 | KCADLIIRS |
| 28376 | EKRLEGWVY |
| 28377 | KDGRNPHCL |
| 28378 | QSRCVDNTV |
| 28379 | ATMNLSWGL |
| 28380 | YSDPKNMSI |
| 28381 | PDLWYEKSS |
| 28382 | TFVRKPSLM |
| 28383 | PYGPIEYDE |
| 28384 | EPRLYCAQE |
| 28385 | DRKQADQIY |
| 28386 | KGNPLDGDT |
| 28387 | GLFEGSPGA |
| 28388 | DIEFLDCEN |
| 28389 | EIHMFGQSE |
| 28390 | WGGMPNNQG |
| 28391 | SVLHVQALA |
| 28392 | ASMHEETCL |
| 28393 | MHGIARMQQ |
| 28394 | EAGCKMWPT |
| 28395 | KMAGIGCWY |
| 28396 | AVYLDHVFS |
| 28397 | GHLFNNNEW |
| 28398 | SNSHPFQSH |
| 28399 | LSAQRLVCD |
| 28400 | TVNWYSGFG |
| 28401 | YIGMKSCSG |
| 28402 | NHSLCWDSK |
| 28403 | PIWQGYWPW |
| 28404 | AELFYDAMY |
| 28405 | CQVNEEYDW |
| 28406 | DTEAIMVVD |
| 28407 | ESWRTLCQI |
| 28408 | GHHNTPDMQ |
| 28409 | GWDKKQNCG |
| 28410 | IKADVDAME |
| 28411 | LTTTVPTKQ |
| 28412 | MAEPVKRCK |
| 28413 | MCHKVMDII |
| 28414 | MSECKNPAY |
| 28415 | TEVEISASF |
| 28416 | TTVAGPHTT |
| 28417 | VSFWYDCHH |
| 28418 | AACYDYKDH |
| 28419 | AAKLPVAKG |
| 28420 | AEFLTYEQK |
| 28421 | AFWQDLYYV |
| 28422 | AGGGSVQCN |
| 28423 | AHLNCDKSM |
| 28424 | AMATCQMQH |
| 28425 | AMGRLHDSK |
| 28426 | AMMLTDLML |
| 28427 | APHWMEISS |
| 28428 | AVMWTVLHF |
| 28429 | CADWACMSL |
| 28430 | CAVKQASAM |
| 28431 | CCWFVYEST |
| 28432 | CFTESITDC |
| 28433 | CIIVRSTAA |
| 28434 | CIKYNSSIG |
| 28435 | CQSNKQQFC |
| 28436 | CVFSWNNYQ |
| 28437 | CYEITMNQG |
| 28438 | DCLTSECAY |
| 28439 | DFETWDWFG |
| 28440 | DFNHIAWWQ |
| 28441 | DHHGIDTTL |
| 28442 | DIWLWDTFA |
| 28443 | DRPNHSTDG |
| 28444 | DSMFIIMNF |
| 28445 | DSMFTIMNF |
| 28446 | DVASWCQCP |
| 28447 | DYENCFESY |
| 28448 | DYGYVSINF |
| 28449 | DYTTHWQYA |
| 28450 | EEKYQPCLK |
| 28451 | EFKRMTRNK |
| 28452 | EGTQTHSAT |
| 28453 | EIKFPACGL |
| 28454 | EKLNKTWAG |
| 28455 | ENDHRDYNI |
| 28456 | ENYMCYHSL |
| 28457 | EQAPLDVMF |
| 28458 | ETMEHKKRE |
| 28459 | ETMQHKKRA |
| 28460 | ETMQHKKRE |
| 28461 | EVADENGNY |
| 28462 | EVCMAVVDN |
| 28463 | FKRICWEAG |
| 28464 | FNLSSCYHQ |
| 28465 | FTEYEHRAS |
| 28466 | FVAANQNHM |
| 28467 | GAAVSRKGY |
| 28468 | GAEQHTNWP |
| 28469 | GDIRTYPAE |
| 28470 | GFTQNYAQN |
| 28471 | GLIHSTHTV |
| 28472 | GMTNEDQAA |
| 28473 | GNPHLIEHC |
| 28474 | GRAHGNVRI |
| 28475 | GRVLLMAQD |
| 28476 | GSFPCYSLG |
| 28477 | GSFPCYSLS |
| 28478 | GSTEVFYMI |
| 28479 | GTAVSKQSK |
| 28480 | GTNHSAEVY |
| 28481 | GTRATKHHV |
| 28482 | HAPPHGTWA |
| 28483 | HAVNDHSWM |
| 28484 | HCKVKECQY |
| 28485 | HDEWLGHGM |
| 28486 | HIDRPPCCM |
| 28487 | HWMEQPPYG |
| 28488 | IQCNLCEAW |
| 28489 | IQYNLCEAW |
| 28490 | IWGICVHYF |
| 28491 | KENTTNRID |
| 28492 | KHHMFVDKH |
| 28493 | KKDEWHCYP |
| 28494 | KMCWWPEAM |
| 28495 | KQNASDQVH |
| 28496 | KQVDPLDFW |
| 28497 | KSDFRVLHQ |
| 28498 | KSEYVVGVP |
| 28499 | KTRFHRERS |
| 28500 | KVDAEGSHF |
| 28501 | KVMVVDSVY |
| 28502 | LATMWFCAD |
| 28503 | LCYHFWKVN |
| 28504 | LEMHYKDNC |
| 28505 | LFRSHWDAQ |
| 28506 | LKASLREQW |
| 28507 | LMENYEIQD |
| 28508 | LMQKWMRTC |
| 28509 | LYLPHCHRI |
| 28510 | LYLSHCHRI |
| 28511 | LYMWQNQHV |
| 28512 | MATCYMYVD |
| 28513 | MCSYFNNRE |
| 28514 | MEFYGRAGN |
| 28515 | MESGTLLGV |
| 28516 | MFCEFPKCG |
| 28517 | MFGTKMSQY |
| 28518 | MFREFPKCG |
| 28519 | MIDLGNRGF |
| 28520 | MIDWGGEWG |
| 28521 | MMLHKQKDY |
| 28522 | MMWKENAGW |
| 28523 | MSECKNHAY |
| 28524 | MSNYHITHE |
| 28525 | MTAHKENHP |
| 28526 | MTAQSPLIP |
| 28527 | MTDYFKALC |
| 28528 | NCDHWFGTL |
| 28529 | NDKSWLKHC |
| 28530 | NETDVGTDV |
| 28531 | NQREWHGLS |
| 28532 | NRGYANVAW |
| 28533 | NTFVKNEFW |
| 28534 | NTLTMQSYL |
| 28535 | NTLTMRSYL |
| 28536 | NTNYWFYYD |
| 28537 | NVEGTRMRP |
| 28538 | NVEHSHCVG |
| 28539 | PKLCSHQTE |
| 28540 | PRLQSRKYL |
| 28541 | PSCLTQESI |
| 28542 | PSTTIMNVM |
| 28543 | PTFGWMNWF |
| 28544 | QCERYHAAD |
| 28545 | QCRLDMQCP |
| 28546 | QFEDYWAIN |
| 28547 | QGQKQNTTM |
| 28548 | QHFVQGTYP |
| 28549 | QICPQSMMP |
| 28550 | QKDERHCYP |
| 28551 | QMFMQMSGH |
| 28552 | QNSRHKYAW |
| 28553 | QTLGCEMGT |
| 28554 | QTLGWEMGT |
| 28555 | QTVETYAAH |
| 28556 | QVGQYQNTI |
| 28557 | RANDGYYIN |
| 28558 | RDGFQEFTP |
| 28559 | RFTGMSDQD |
| 28560 | RNMSRQEGM |
| 28561 | RRFVQGTYP |
| 28562 | RTMGMDWFT |
| 28563 | RVAMQQMNH |
| 28564 | SAWQWAQMN |
| 28565 | SCAVCGDCD |
| 28566 | SCTARAWIC |
| 28567 | SESQEGVYG |
| 28568 | SGALKNREG |
| 28569 | SKYHWHMYH |
| 28570 | SRDAAAYSA |
| 28571 | STEWAALGV |
| 28572 | STFISSGIR |
| 28573 | SYIPFDTQK |
| 28574 | TAAGYIVDV |
| 28575 | TAYMIDMYC |
| 28576 | TCNVSWKHA |
| 28577 | TCSYFNNRE |
| 28578 | TGKVDGMQY |
| 28579 | TLPKMYMWP |
| 28580 | TMRTTMCIK |
| 28581 | TMRTTMCIT |
| 28582 | TPGINRHCC |
| 28583 | TTFITETMD |
| 28584 | TTHQTAFEW |
| 28585 | TVNEALTCH |
| 28586 | TVRWCHWCL |
| 28587 | TWAMHPSHE |
| 28588 | VAWSCDKFA |
| 28589 | VEDMGQVEA |
| 28590 | VGQNFNTGR |
| 28591 | VGTDCATSL |
| 28592 | VPSLNTSAG |
| 28593 | VQKITDEVC |
| 28594 | VSDDYECGI |
| 28595 | VTQWKYGDT |
| 28596 | VTRNEIWFP |
| 28597 | VTYTNGMAE |
| 28598 | VVTVHVRGH |
| 28599 | WQIGGCPEE |
| 28600 | WTHTYATGR |
| 28601 | YAEVAVQQE |
| 28602 | YAMPLYFWE |
| 28603 | YAPQNEKPE |
| 28604 | DFKFHSTGS |
| 28605 | MMVTKEVNL |
| 28606 | RTEGWFKYD |
| 28607 | TSMWGLETS |
| 28608 | FPGVMEGEI |
| 28609 | TAKQHFMVW |
| 28610 | FKMDCHSCG |
| 28611 | ATWPERWQN |
| 28612 | LMENYEFQD |
| 28613 | ESYHYKLMT |
| 28614 | QPIHFQQRD |
| 28615 | VVNQQEFYY |
| 28616 | AQKITDEVC |
| 28617 | DVRSSMDHC |
| 28618 | FNLSFCYHQ |
| 28619 | RWHNGKGPM |
| 28620 | RYSNVAHHA |
| 28621 | FAMEQVKCD |
| 28622 | DRVMISIQP |
| 28623 | AGSGSVQCN |
| 28624 | LPTLRVADK |
| 28625 | DTDDWTCVF |
| 28626 | SWHNNWTMD |
| 28627 | WHDMVNSGP |
| 28628 | RQCTDEECW |
| 28629 | RVVMQQMNH |
| 28630 | DRLNHSTDG |
| 28631 | FLIQGDLTF |
| 28632 | FPPIVKRHH |
| 28633 | LCEGQQWHA |
| 28634 | SGSIYHHMC |
| 28635 | VVMWQFDSQ |
| 28636 | RPDLNEIEC |
| 28637 | SADNKIGGP |
| 28638 | HAYQTMSEQ |
| 28639 | CFTEPITDC |
| 28640 | MTFVCNFGT |
| 28641 | TSCGYNDNN |
| 28642 | HPMFPNTED |
| 28643 | AFAAYCSRE |
| 28644 | CAKFTNAIH |
| 28645 | NHQDNSAYP |
| 28646 | IWGIYVHYF |
| 28647 | YWWGFCWGL |
| 28648 | KVEGYGWTR |
| 28649 | YDQYGDRTM |
| 28650 | EIGIIRHGH |
| 28651 | VDHTLDFYH |
| 28652 | TVVINACLE |
| 28653 | LRHWFFSNM |
| 28654 | AMTRQNGWM |
| 28655 | WMCEFSCIQ |
| 28656 | LTTKDDKEQ |
| 28657 | PECKGAVDP |
| 28658 | FQTPVANVC |
| 28659 | QVGQYQNMI |
| 28660 | QNADQAMSG |
| 28661 | RFVLCDYME |
| 28662 | HSEMHNHHA |
| 28663 | TAFANAHTF |
| 28664 | KVGCFVDVW |
| 28665 | NWRMVHGRW |
| 28666 | GVNDLQNIC |
| 28667 | CVVNRGSIT |
| 28668 | DTHTCSGFS |
| 28669 | DFEMWDWFG |
| 28670 | TRRSWECAP |
| 28671 | SCTTRAWIC |
| 28672 | MIEPNDHGK |
| 28673 | KKDERHCYP |
| 28674 | CHCQMEAEW |
| 28675 | RVEMQYWEN |
| 28676 | DWTINDGWS |
| 28677 | TVREDCCIN |
| 28678 | SSQKYKDWE |
| 28679 | CVWSVGAEW |
| 28680 | DTAATMEPK |
| 28681 | EPRPETEGC |
| 28682 | MVTERKPWS |
| 28683 | SYDKEHAVW |
| 28684 | RANTWTRRH |
| 28685 | HLTILHCPE |
| 28686 | YCGWKEFRR |
| 28687 | MASFVEHCI |
| 28688 | KAMGMLEWA |
| 28689 | EEKDPHMDS |
| 28690 | MDNNYHQFG |
| 28691 | WIQWVRKMG |
| 28692 | NEWSRWLTG |
| 28693 | FQNVEIEKF |
| 28694 | QNMFGNQPY |
| 28695 | CKQRNCDGN |
| 28696 | LCNNADMRC |
| 28697 | VKQSMPMKG |
| 28698 | MANLKHQLA |
| 28699 | EDFVTTSEL |
| 28700 | LKRTVGVCQ |
| 28701 | ALILYARKH |
| 28702 | ASIKFEGQE |
| 28703 | SQKQFSMQH |
| 28704 | CDNAGEFGQ |
| 28705 | NRCQFGKDW |
| 28706 | VTNIFNGPR |
| 28707 | GYKCGCVCC |
| 28708 | ACEQIFEGI |
| 28709 | AHGWSQGAS |
| 28710 | AHGWSQGES |
| 28711 | AHRWSQGES |
| 28712 | AIHEVQSSF |
| 28713 | AILKITKVL |
| 28714 | AMINPKNDH |
| 28715 | AMINPNNDH |
| 28716 | AMTNPNNDH |
| 28717 | ANEEMMAAD |
| 28718 | APAPTLREW |
| 28719 | AQSPGIHQE |
| 28720 | AQSPGMHQE |
| 28721 | ATDQPANLC |
| 28722 | ATDQPVNLC |
| 28723 | ATGTQLNQD |
| 28724 | AVHNVKDVH |
| 28725 | CASSYPRDL |
| 28726 | CASYYPRAL |
| 28727 | CASYYPRDL |
| 28728 | CEFEKMDYG |
| 28729 | CEFKKMDYG |
| 28730 | CEFKKVDYG |
| 28731 | CFCKYDMQM |
| 28732 | CFCKYEMQM |
| 28733 | CFCTYDMQM |
| 28734 | CGMCHECTL |
| 28735 | CGTAYWTAY |
| 28736 | CGTTYWTAY |
| 28737 | CPKAWEKAA |
| 28738 | CPKDWAKAA |
| 28739 | CPKDWEKAA |
| 28740 | CPKDWGKAA |
| 28741 | CPKGWEKAA |
| 28742 | CQMLLHAFK |
| 28743 | CQMLLRAFK |
| 28744 | CQTETILCG |
| 28745 | CQTKTILCG |
| 28746 | CQTKTMLCG |
| 28747 | CTKECMLWP |
| 28748 | CTKEYMLWP |
| 28749 | CVECNQPVR |
| 28750 | CVMCHACTL |
| 28751 | CVMCHECTL |
| 28752 | DAGCMGGWF |
| 28753 | DCARQHYYA |
| 28754 | DICLEPAQT |
| 28755 | DICLETAQT |
| 28756 | DICLETTQT |
| 28757 | DICSETAQT |
| 28758 | DKSHVDQDK |
| 28759 | DLYTYIMEN |
| 28760 | DLYTYIMGN |
| 28761 | DLYTYLMEN |
| 28762 | DLYTYMMEN |
| 28763 | DLYTYVMEN |
| 28764 | DMEEHTHSC |
| 28765 | DNLCIGAFG |
| 28766 | DNLCIGASG |
| 28767 | DNPCIGAFG |
| 28768 | DQRMLLKEL |
| 28769 | DQRMLLKGL |
| 28770 | DSCSIKCDE |
| 28771 | DSCSITCDE |
| 28772 | DSCTHHEGF |
| 28773 | DSHTANFRG |
| 28774 | DSHTVNFRG |
| 28775 | DSYTHHEGF |
| 28776 | DYASLAGQA |
| 28777 | DYASLEGQA |
| 28778 | DYASLEGRA |
| 28779 | DYASLGGQA |
| 28780 | DYASLVGQA |
| 28781 | DYGPKDFFA |
| 28782 | DYGPKEFFA |
| 28783 | EAIFAMTST |
| 28784 | EAYAMKQNT |
| 28785 | EDQVASHWI |
| 28786 | EFMCVDQLE |
| 28787 | EFMCVDQSE |
| 28788 | EGVIISLLE |
| 28789 | EMMQTCVQH |
| 28790 | EMMRTCVQH |
| 28791 | EQICSEIDQ |
| 28792 | EQVMDTWQL |
| 28793 | ESLSGGEHP |
| 28794 | ESLSNGEHP |
| 28795 | ESLSSGEHP |
| 28796 | ESLYSGEHP |
| 28797 | ESQSSGEHP |
| 28798 | ETQYWYARQ |
| 28799 | EVFLESQDE |
| 28800 | EVFRESQDE |
| 28801 | EVKYKGNYN |
| 28802 | FADLILSDG |
| 28803 | FGDKMTVTS |
| 28804 | FSPNTPRSD |
| 28805 | FSPNTQRSD |
| 28806 | FWSHVGLQE |
| 28807 | FWSPVGLQE |
| 28808 | GASYFSQPV |
| 28809 | GCTWEQHAE |
| 28810 | GCTWEQHGE |
| 28811 | GCTWEQHVE |
| 28812 | GKSHVDQDK |
| 28813 | GKSHVEQDK |
| 28814 | GKSHVNQDK |
| 28815 | GKSPVDQDK |
| 28816 | GQDCWGLAM |
| 28817 | GRRYPECTT |
| 28818 | GRWYPECTT |
| 28819 | GSSYFGQPV |
| 28820 | GSSYFIQPV |
| 28821 | GSSYFNQPV |
| 28822 | GSSYFSQPV |
| 28823 | GTSYFSQPV |
| 28824 | GTYGSSFYM |
| 28825 | GVAHYHHHP |
| 28826 | GVAHYHHHQ |
| 28827 | GVAHYHHRQ |
| 28828 | GVAHYQHHQ |
| 28829 | GVAHYRHHQ |
| 28830 | GVAPYHHHQ |
| 28831 | GVFECSTCQ |
| 28832 | GYTWEQHVE |
| 28833 | HAVWRQSSH |
| 28834 | HCWGHAFFF |
| 28835 | HCWGHEFFF |
| 28836 | HCWGHGFFF |
| 28837 | HCYSTSEME |
| 28838 | HCYSTSETE |
| 28839 | HTDDLKKVI |
| 28840 | HTIITNQPY |
| 28841 | HVFSANVDK |
| 28842 | HVFSANVNK |
| 28843 | HVVWRQSSH |
| 28844 | IAHAAMHDG |
| 28845 | IAHEAMHDG |
| 28846 | IAHEAMRDG |
| 28847 | IAHEAMYDG |
| 28848 | IAHGAMHDG |
| 28849 | ICFQHQIRK |
| 28850 | ICFQYQIRK |
| 28851 | ICMAKNGWW |
| 28852 | ICMDKHGWW |
| 28853 | ICMDKNGWW |
| 28854 | ICMDKSGWW |
| 28855 | IIGFMPPTG |
| 28856 | ISNPLMTQM |
| 28857 | ITTAYPECD |
| 28858 | IVAIAHGEH |
| 28859 | IVASAHGEH |
| 28860 | KAIPQGSSC |
| 28861 | KARQDDVNT |
| 28862 | KHDIYHTYT |
| 28863 | KHNPSRHGI |
| 28864 | KQVNFNAKN |
| 28865 | KSDTQCRFF |
| 28866 | KSDTQWRFF |
| 28867 | KTVPECESS |
| 28868 | KTVQECESS |
| 28869 | KTVQEGESS |
| 28870 | KTVQEWESS |
| 28871 | LAAMQQGPW |
| 28872 | LAMRRFDNF |
| 28873 | LCFQHQIRK |
| 28874 | LFNKNDCSM |
| 28875 | LFNKNGCSM |
| 28876 | LFNKNGCSV |
| 28877 | LFRAYAWFQ |
| 28878 | LGRKPYALD |
| 28879 | LGSDFNARD |
| 28880 | LLTGQNSSH |
| 28881 | LMWIALEAW |
| 28882 | LPWIHTNAN |
| 28883 | LSHKPADPT |
| 28884 | LSMQWAVIH |
| 28885 | LSMQWAVLH |
| 28886 | LSMQWVVLH |
| 28887 | LTCYHNELS |
| 28888 | LVMRRFDNF |
| 28889 | MADLIVEHD |
| 28890 | MAEHRKQEH |
| 28891 | MCHRASDQN |
| 28892 | MCQRASDQN |
| 28893 | MDGQAWKEA |
| 28894 | MGHKPVLSS |
| 28895 | MIRADIRLI |
| 28896 | MIRAVIRLI |
| 28897 | MIREAIRLI |
| 28898 | MIREDIRLI |
| 28899 | MIREGIRLI |
| 28900 | MIREVIRLI |
| 28901 | MIREVMRLI |
| 28902 | MIRGVIRLI |
| 28903 | MIRVVIRLI |
| 28904 | MLTGQNSSH |
| 28905 | MLYICMMAT |
| 28906 | MNMCFGQST |
| 28907 | MSMQWAVLH |
| 28908 | MTTVIYDSC |
| 28909 | MVEMDHWAK |
| 28910 | MVETDHWAK |
| 28911 | MYQRASDQN |
| 28912 | NATSIYDID |
| 28913 | NATSIYDVD |
| 28914 | NFEMQPRAD |
| 28915 | NFMLVNGPL |
| 28916 | NNEPYQKFN |
| 28917 | NNEPYRKFN |
| 28918 | NNFNDAAGL |
| 28919 | NNFNDEAGL |
| 28920 | NPDYRNEMP |
| 28921 | NTRPFEKDI |
| 28922 | PERRIGQWY |
| 28923 | PGICKANWT |
| 28924 | PGIRKANWT |
| 28925 | PMFPDSMSS |
| 28926 | PMSPDSMSS |
| 28927 | QCSYCQQVY |
| 28928 | QHVPVGPPA |
| 28929 | QHVQVGPPA |
| 28930 | QIMWFPPEE |
| 28931 | QLCSPPMLD |
| 28932 | QLCYPPMLD |
| 28933 | QLERTGVHP |
| 28934 | QLHYYNVGH |
| 28935 | QMFQGLWSH |
| 28936 | QPERTGVHP |
| 28937 | QQERFPHSW |
| 28938 | QRVQVGPPA |
| 28939 | QSDTQCRFF |
| 28940 | RHVQVGPPA |
| 28941 | RIHYELYIC |
| 28942 | RPEAVICWN |
| 28943 | RPEDVICWN |
| 28944 | SAAAVPSWH |
| 28945 | SAADVPSWH |
| 28946 | SAAVKSEPP |
| 28947 | SADVKSEPP |
| 28948 | SAHMYHAGY |
| 28949 | SATQNWNEH |
| 28950 | SATVKSEPP |
| 28951 | SDTWAMYHH |
| 28952 | SGTWAMYHH |
| 28953 | SHATFMMGN |
| 28954 | SKSHVDQDK |
| 28955 | SRDWPASMN |
| 28956 | SRDWPESMN |
| 28957 | SRDWSESMN |
| 28958 | SSPNTQRSD |
| 28959 | STHIYWICP |
| 28960 | STHIYWICS |
| 28961 | STHVYWICP |
| 28962 | STVPQMRWT |
| 28963 | SVSPHTPMH |
| 28964 | SVSQHTPMH |
| 28965 | SYCPVVQWD |
| 28966 | SYCQVVQWD |
| 28967 | TDPLVMMEH |
| 28968 | TECLCVYNF |
| 28969 | TLSSGARDM |
| 28970 | TMMFEQNSK |
| 28971 | TTDQPANLC |
| 28972 | TVETDHWAK |
| 28973 | VAEHRKQEH |
| 28974 | VAEHRQQEH |
| 28975 | VDGQAWKEA |
| 28976 | VKEQTYAAN |
| 28977 | VLAGLECQY |
| 28978 | VNEEMMAAD |
| 28979 | VTDRNAWVG |
| 28980 | VTEHRKQEH |
| 28981 | VTGTQLNQD |
| 28982 | VTNRWKRKS |
| 28983 | VTNRWKRMS |
| 28984 | VVECASHSG |
| 28985 | VVNQFEHWM |
| 28986 | WIHSELYIC |
| 28987 | WIHYELYIC |
| 28988 | WSFSERPGS |
| 28989 | WTGYSWSVA |
| 28990 | YAVMSHPAG |

TABLE 81
Sequences of the 581 to 589 Region in AAV5 VP1
Capsid Polypeptide that Drive Skeletal
Muscle Tissue Tropism

| SEQ | |
|---|---|
| ID | 581-589 |
| NO | Sequence |
| 30991 | ACIFVMLPL |
| 30992 | ACIFVVLPL |
| 30993 | AGKEVSAGM |
| 30994 | AKKGMRHDQ |
| 30995 | AKKGMWHDQ |
| 30996 | APSKPWLHF |
| 30997 | ARLCKRKQA |
| 30998 | AWVEHAAFM |
| 30999 | AYIFVVLPL |
| 31000 | CAWDKHHKM |
| 31001 | CAWDKHHKV |
| 31002 | CDPHWAWCE |
| 31003 | CDPHWDWCE |
| 31004 | CDPHWDWWE |
| 31005 | CDPHWGWCE |
| 31006 | CFVHTSCQN |
| 31007 | CPWFGLCNW |
| 31008 | CPWFSLCNW |
| 31009 | CPWFSLWNW |
| 31010 | CRTCDEDGA |
| 31011 | CRTCDEDVA |
| 31012 | DDCQFDNEN |
| 31013 | DDFGTLPRM |
| 31014 | DENIKIIYG |
| 31015 | DENIKVIYG |
| 31016 | DIDPLRNSG |
| 31017 | DIDQLRNSC |
| 31018 | DIDQLRNSG |
| 31019 | DKLKFVKQW |
| 31020 | DKPKFVKQW |
| 31021 | DLRSPGFYA |
| 31022 | DMTPKNMQM |
| 31023 | DRAMYHKVV |
| 31024 | DRAMYRKVG |
| 31025 | DRAMYRKVV |
| 31026 | DRTMYRKVV |
| 31027 | DVYQFWTSF |
| 31028 | EACYMCRNV |
| 31029 | EAYDVAAVN |
| 31030 | EAYDVATVN |
| 31031 | EFECFHNGP |
| 31032 | EFGCFHNGP |
| 31033 | ELNLPVRWY |
| 31034 | ERRMEVLNA |
| 31035 | ETYDVAAVN |
| 31036 | EVECFHNGP |
| 31037 | FTDTHEMVL |
| 31038 | FYPRPELTG |
| 31039 | GCIQFESTG |
| 31040 | GDGYLEDSW |
| 31041 | GMAPKNMQM |
| 31042 | GMTPKNMQM |
| 31043 | GNKDCYGSM |
| 31044 | GRNFQESLQ |
| 31045 | GTMLTTEDL |
| 31046 | GVWQIHARE |
| 31047 | GVWQIPARE |
| 31048 | GVWRIHARE |
| 31049 | GYTTMQTLH |
| 31050 | HLDAIWPLW |
| 31051 | HLDEIWPLW |
| 31052 | HLDEIWPSW |
| 31053 | HLDESWPLW |
| 31054 | HMQWMGFRG |
| 31055 | HSRCSHYDW |
| 31056 | HTPHDDYRS |
| 31057 | HYMSMTTYE |
| 31058 | IAYTLQFQW |
| 31059 | ICNEQEFVA |
| 31060 | ICNEQEVVA |
| 31061 | ICNKQEFVA |
| 31062 | IDHCGCTQP |
| 31063 | IFRCFGVEY |
| 31064 | ILARWSEKE |
| 31065 | IPAFPLRMV |
| 31066 | IPEFPLRMV |
| 31067 | IPEFPLRRV |
| 31068 | IVMYPFCST |
| 31069 | KENLKTNWW |
| 31070 | KQEGDYVDM |
| 31071 | KVKACEGSK |
| 31072 | KVKSCAGSK |
| 31073 | KVKSCEESK |
| 31074 | KVKSCEGAK |
| 31075 | KVKSCEGSK |
| 31076 | KVKSWEGSK |
| 31077 | KVRSCEGSK |
| 31078 | KWMQDITYF |
| 31079 | LALNAPNFC |
| 31080 | LELPDSIKV |
| 31081 | LELPVSIKV |
| 31082 | LLPLFTPYV |
| 31083 | LMWIGQVVE |
| 31084 | LMWTGQVVE |
| 31085 | LQAQEQFHI |
| 31086 | LRNCICNDH |
| 31087 | LRTMVQNDH |
| 31088 | LRTMVRNDH |
| 31089 | LTGWFLIWV |
| 31090 | LVLNAPNFC |
| 31091 | LWTMVQNDH |
| 31092 | MAGYNDSCM |
| 31093 | MAGYNDSWM |
| 31094 | MAGYNGSCM |
| 31095 | MAGYSDSCM |
| 31096 | MIEWQFCTS |
| 31097 | MMIGNQMHC |
| 31098 | MPGYNDSCM |
| 31099 | MRGNCEIKR |
| 31100 | MTGYNDSCM |
| 31101 | NAPCMWMPD |
| 31102 | NAPCMWMPY |
| 31103 | NAPCMWMSY |
| 31104 | NDFHIEPIH |
| 31105 | NEDPGAGCN |
| 31106 | NEDPGAGCY |
| 31107 | NGFHIEPIH |
| 31108 | NGFRIEPIH |
| 31109 | NLQSMFCEP |
| 31110 | NQPSSDDRQ |
| 31111 | NVAQPTIHR |
| 31112 | NVTQPTIHR |
| 31113 | PFECEANEQ |
| 31114 | PFGCEANEQ |
| 31115 | PHEQKMLDR |
| 31116 | PPCGLKCMN |
| 31117 | PPCGLKCMS |
| 31118 | PQAQEQFHI |
| 31119 | QFFHTLWKY |
| 31120 | QFSHTLWKY |
| 31121 | QNWHMKNKM |
| 31122 | QNWHMKNNM |
| 31123 | QSTQICHNR |
| 31124 | RFWPRKVQP |
| 31125 | RFWQRKVQP |
| 31126 | RKDDQMYAL |
| 31127 | RKEGCTSHG |
| 31128 | RSGMARYAY |
| 31129 | RTMLTTEDL |
| 31130 | SHYFCTMST |
| 31131 | SPPCCKKIL |
| 31132 | SWKLKLCQT |
| 31133 | SWRLKLCQT |
| 31134 | TCCLPWQRI |
| 31135 | TGKEASAGM |
| 31136 | TGKEDSAGM |
| 31137 | TGKEGSAGM |
| 31138 | TGKEVSAGM |
| 31139 | TKKGMRHDQ |
| 31140 | TSFSFPDCM |
| 31141 | TVSCGPADM |
| 31142 | TVSCGPTDM |
| 31143 | TWVEHAAFM |
| 31144 | VDLHHPIYR |
| 31145 | VVKFSWKPW |
| 31146 | VVPQPPLET |
| 31147 | VVQFSWKPW |
| 31148 | WNPHHDFEA |
| 31149 | WNPHHDFED |
| 31150 | WNPHHDFEG |
| 31151 | WNPHRDFED |
| 31152 | YGNCRGHRL |
| 31153 | YGNCWGHRL |
| 31154 | YIDQLRNSG |
| 31155 | YNPYRFINK |
| 31156 | YNPYRFMNK |
| 31157 | AENLPRWGM |
| 31158 | CIETGILNG |
| 31159 | CTQTTVRWP |
| 31160 | EKVHCMWWH |
| 31161 | ETHEMNVCM |
| 31162 | FCPAEWGCL |
| 31163 | GRHWAFCDY |
| 31164 | HCSRHFCEV |
| 31165 | HQQSEAFVS |
| 31166 | IACMGEVQG |
| 31167 | KAHQTVQDK |
| 31168 | KCVSMDFSG |
| 31169 | MYEYGQDWY |
| 31170 | NAISVGEWD |
| 31171 | NKRNLYALQ |
| 31172 | NMVHENRFW |
| 31173 | PAWDLVGML |
| 31174 | PKGMAKICE |
| 31175 | QKKGIEAVD |
| 31176 | RFRFRAQVW |
| 31177 | RLERDNNEA |
| 31178 | VIKWRSQDK |
| 31179 | WLANSWFRE |
| 31180 | YMWWQEGTL |
| 31181 | YVVGDAACP |
| 31182 | KFAMDWVSD |
| 31183 | LNELPRAEY |
| 31184 | ISLQEMMGK |
| 31185 | VGECVASAP |
| 31186 | MREMPMVGA |
| 31187 | AAANYDQIE |
| 31188 | AACCPFAIM |
| 31189 | AAGMSGQFE |
| 31190 | AANEKPTHQ |
| 31191 | AAVQFVLSC |
| 31192 | ACMSRAMFD |
| 31193 | ACQCKVIWQ |
| 31194 | ACTWPGVGP |
| 31195 | ADQSWYIKG |
| 31196 | AEIYNCEFK |
| 31197 | AEVEADQRM |
| 31198 | AFANALACY |
| 31199 | AFEYFGNPI |
| 31200 | AFNRIGRYT |
| 31201 | AFYYLGHDS |
| 31202 | AGSTAMQSQ |
| 31203 | AGTSFDMMP |
| 31204 | AHMENELEM |
| 31205 | AHNYSLAEQ |
| 31206 | AHRQLDAEW |
| 31207 | AIAWWHEPM |
| 31208 | AIDWEDVWE |
| 31209 | AIEMQRQTD |
| 31210 | AIFEPRRWH |
| 31211 | AIQLHFEWG |
| 31212 | AKGNTNNNA |
| 31213 | ALVHKDLYG |
| 31214 | AMMELMWTD |
| 31215 | ANHHCGLAC |
| 31216 | ANHLFVIEH |
| 31217 | APDDENCDF |
| 31218 | AQATESSQP |
| 31219 | AQVEYTYET |
| 31220 | AQYPTLEYA |
| 31221 | ASFFSSVAQ |
| 31222 | ATDALSTSL |
| 31223 | ATDNHWGHG |
| 31224 | ATENVMWTC |
| 31225 | ATGLHITTC |
| 31226 | ATHERDFFV |
| 31227 | ATHEVITPA |
| 31228 | ATNWGPMSP |
| 31229 | ATQEFLGLN |
| 31230 | ATVATYINT |
| 31231 | AVGEVDAYS |
| 31232 | AVIYSSYNW |
| 31233 | AVMYYGYES |
| 31234 | AVWIEPRQW |
| 31235 | AVYLNEFNM |
| 31236 | AWFCPLLYN |
| 31237 | AWSARSVNM |
| 31238 | AYGWENQFE |
| 31239 | AYHELTSCA |
| 31240 | CADSKMTST |
| 31241 | CAHFAMNNH |
| 31242 | CAVCPALSA |
| 31243 | CCRHWRPTP |
| 31244 | CEHDMCWPT |
| 31245 | CEQVQIGDE |
| 31246 | CFICCHCFQ |
| 31247 | CGEHDKFKS |
| 31248 | CGGCEDWPM |
| 31249 | CHMYHRREG |
| 31250 | CHPHDEHKT |
| 31251 | CHQENFVFR |
| 31252 | CIASFTDGK |
| 31253 | CKVNESAKN |
| 31254 | CNPRPQMVG |
| 31255 | CNTKFLSQS |
| 31256 | CQKTFELHG |
| 31257 | CQMPERWTN |
| 31258 | CSGRGEIEF |
| 31259 | CTCDYTAQY |
| 31260 | CTCEGGSTA |
| 31261 | CVLGHIHHG |
| 31262 | CVMQRAADL |
| 31263 | CVQPHVDPM |
| 31264 | CVTCTNVYG |
| 31265 | CWSQEANRG |
| 31266 | CWTDNDDMD |
| 31267 | CYGHLNAMC |
| 31268 | DAEHSPNIM |
| 31269 | DAKHHQMVH |
| 31270 | DAKPYQRNI |
| 31271 | DATATGQWS |
| 31272 | DAWTMASAM |
| 31273 | DCEEFDRTP |
| 31274 | DCKWEHHSD |
| 31275 | DCPQDEQMN |
| 31276 | DDEPKTDWC |
| 31277 | DDMWQRDWF |
| 31278 | DDPAGHCYV |
| 31279 | DEHKRLDIC |
| 31280 | DEVPNKCDC |
| 31281 | DFKPKQHDH |
| 31282 | DFNWQDSGY |
| 31283 | DFSSINCQS |
| 31284 | DGMCNGGHP |
| 31285 | DGVSDHQRP |
| 31286 | DHTDMLCFG |
| 31287 | DIFPNCKTD |
| 31288 | DIFPSMNMS |
| 31289 | DIFQLTDDS |
| 31290 | DIHEWQVMS |
| 31291 | DIHGSQEFA |
| 31292 | DINENMSVE |
| 31293 | DIVCKFASP |
| 31294 | DLWRTFEAQ |
| 31295 | DNHDCGRSH |
| 31296 | DNHGYDRHY |
| 31297 | DNRCCWHMN |
| 31298 | DNRWWVAFS |
| 31299 | DNVIMTTVG |
| 31300 | DPSNCMRIM |
| 31301 | DQEVCTAMD |
| 31302 | DQLNVNESG |
| 31303 | DQNIFLSID |
| 31304 | DRSDGGFVG |
| 31305 | DSGEGLHNK |
| 31306 | DSLLMREAY |
| 31307 | DSRRIYAWI |
| 31308 | DSYCVNHFH |
| 31309 | DTAYVWCEM |
| 31310 | DTNRCMMQS |
| 31311 | DTQLDDIAG |
| 31312 | DTYPMQSAD |
| 31313 | DVDDLVEIM |
| 31314 | DVEDHDSQQ |
| 31315 | DVIANIHSR |
| 31316 | DVMHNHVKA |
| 31317 | DYRQYLEVQ |
| 31318 | EADKQYDDQ |
| 31319 | EAGMKQLCA |
| 31320 | EAIQFPSRG |
| 31321 | EAQDKWAGA |
| 31322 | EAQSIACPT |
| 31323 | EAYMMVCSH |
| 31324 | ECLFLRNVE |
| 31325 | ECNRMAMAQ |
| 31326 | ECNVNPACF |
| 31327 | ECSDWTHMQ |
| 31328 | ECWRHVQWN |
| 31329 | ECYSIYCEE |
| 31330 | EDNIRIMMI |
| 31331 | EDYHYTHTE |
| 31332 | EEAYVNCSV |
| 31333 | EFDEFNTRG |
| 31334 | EFESGLLDH |
| 31335 | EFHSYCMSF |
| 31336 | EFIHIDREC |
| 31337 | EFKCVDATE |
| 31338 | EFMEAHCGC |
| 31339 | EFMPEISTG |
| 31340 | EFRHLKHGG |
| 31341 | EGLENNDMF |
| 31342 | EHAGCQLWS |
| 31343 | EHEQLPSCY |
| 31344 | EHFFDDIHG |
| 31345 | EHKLVMCFP |
| 31346 | EHKWYTTLG |
| 31347 | EIHHFEYWS |
| 31348 | EIIHVVGND |
| 31349 | EILKNYTEA |
| 31350 | EIRPQDTEM |
| 31351 | EKAKRISGW |
| 31352 | EKPMMQNEC |
| 31353 | EKYSIIAHS |
| 31354 | ELAYLWPPH |
| 31355 | ELHSVNYML |
| 31356 | EMCVQHNTS |
| 31357 | EMEAKAFQD |
| 31358 | EMFYCGAYH |
| 31359 | EMMASAQSN |
| 31360 | EMMPLHQQP |
| 31361 | ENFSFYKCT |
| 31362 | ENIHYACSN |
| 31363 | ENLVINHIS |
| 31364 | ENNKWYSAW |
| 31365 | ENRCFSDEQ |
| 31366 | EPHWFFAES |
| 31367 | EPQATQKAI |
| 31368 | EQRIVQQAT |
| 31369 | ERCESHRAS |
| 31370 | ERQTCWRGS |
| 31371 | ESADMACSQ |
| 31372 | ESHSYVIAA |
| 31373 | ESRCVLEQG |
| 31374 | ESTNSHWGA |
| 31375 | ESVDVGLMH |
| 31376 | ETEPHTFFS |
| 31377 | ETGIDNNAT |
| 31378 | ETNLECGQK |
| 31379 | ETQYCRGQQ |
| 31380 | EVAHKLKHA |
| 31381 | EVDEVSPSA |
| 31382 | EVGTNPSKN |
| 31383 | EVRVDPCCL |
| 31384 | EVSHDAQCT |
| 31385 | EVTEVQTKA |
| 31386 | EWAMWKPYA |
| 31387 | EWSDEHLKA |
| 31388 | EWVREDGLP |
| 31389 | EYGVVKTPP |
| 31390 | EYMNYEHES |
| 31391 | EYSQNHSEV |
| 31392 | FAAHYGQND |
| 31393 | FAAPNSYWP |
| 31394 | FAKTDVWCG |
| 31395 | FANQGAIHP |
| 31396 | FCPEHWFNI |
| 31397 | FDMFASPDD |
| 31398 | FDNSCPKRW |
| 31399 | FFALNPNYP |
| 31400 | FFELSQTVF |
| 31401 | FGGKIDIGL |
| 31402 | FHCRGYLGQ |
| 31403 | FHHAMTEWQ |
| 31404 | FKPVNAPQL |
| 31405 | FLESWEKDQ |
| 31406 | FLPPNCIEW |
| 31407 | FLWLMQAIN |
| 31408 | FMEQYMMVD |
| 31409 | FMGQQNVAC |
| 31410 | FPQHMNVCV |
| 31411 | FRAQLPTCS |
| 31412 | FRGIETGQG |
| 31413 | FSGCYCMRI |
| 31414 | FYGQVTKSC |
| 31415 | FYQTIGQQE |
| 31416 | GAFMDMLIS |
| 31417 | GAIDYHTQM |
| 31418 | GAMLAQLSC |
| 31419 | GASWNDREH |
| 31420 | GEPELPNNA |
| 31421 | GFFIYQNRI |
| 31422 | GGARALRFN |
| 31423 | GGIQLDREW |
| 31424 | GHRKAFLAT |
| 31425 | GIFQYMSKH |
| 31426 | GIGPIMSAM |
| 31427 | GIIQVRMEW |
| 31428 | GINQEYTKC |
| 31429 | GITLTELQF |
| 31430 | GLRHDPAQF |
| 31431 | GMAYLQNEG |
| 31432 | GMQWMDRQA |
| 31433 | GMVQKQHGR |
| 31434 | GNVVWLARP |
| 31435 | GPDGAKRNK |
| 31436 | GPIGMDWED |
| 31437 | GPISGRYSV |
| 31438 | GPVVSPCKP |
| 31439 | GQFEDLLVL |
| 31440 | GQGLKAWPC |
| 31441 | GQVAFDKDY |
| 31442 | GRHDWMDKE |
| 31443 | GSLFSHFWV |
| 31444 | GSYNRLHDN |
| 31445 | GTDRNYAQC |
| 31446 | GTEQVSAEA |
| 31447 | GVHHNEHCR |
| 31448 | GVKFFEYDQ |
| 31449 | GVMVWNQLR |
| 31450 | GVYMEHNFE |
| 31451 | GVYSNCHDG |
| 31452 | GWYCMRQKI |
| 31453 | GYCGLMQYP |
| 31454 | GYTDDGVGY |
| 31455 | HADHAMAYF |
| 31456 | HASLQCMKT |
| 31457 | HCEGGQTIV |
| 31458 | HDLSYIDMA |
| 31459 | HEDRKHAGE |
| 31460 | HEDVGWCSR |
| 31461 | HEEFEYWNL |
| 31462 | HEHKRSSTE |
| 31463 | HEIDGWQMA |
| 31464 | HFDEITAGT |
| 31465 | HFERSPYWI |
| 31466 | HFQPLFEYN |
| 31467 | HGTEVCHWS |
| 31468 | HHEEHDIFA |
| 31469 | HHSFTIPYQ |
| 31470 | HILHLYICW |
| 31471 | HNFYPVTGG |
| 31472 | HPEEFRGFT |
| 31473 | HPGTYAIHA |
| 31474 | HPHAGGINK |
| 31475 | HPVWVRSDH |
| 31476 | HQETFFHGP |
| 31477 | HRDPHLLME |
| 31478 | HSFLDNNAD |
| 31479 | HSGMDHAQY |
| 31480 | HSMQGAPLY |
| 31481 | HSRWFEDYE |
| 31482 | HSTCPCVVR |
| 31483 | HSYPTAFYY |
| 31484 | HTGMEHMLY |
| 31485 | HTKQMDVDM |
| 31486 | HVIDHTGIL |
| 31487 | HVKINGTGK |
| 31488 | HWYMMEDYV |
| 31489 | HYFTKYKVD |
| 31490 | IAGLFLKTY |
| 31491 | IAMISLYNG |
| 31492 | IANCNTKHL |
| 31493 | IAVQQFYAA |
| 31494 | IAYQLTEIT |
| 31495 | ICFTPMHDS |
| 31496 | ICLPKMYFG |
| 31497 | IDIDMCYDY |
| 31498 | IDPPHKMLD |
| 31499 | IFGCTSAAE |
| 31500 | IGCHRMLNP |
| 31501 | IGNCDTFTK |
| 31502 | IGVVFQRDF |
| 31503 | IHHALQCHK |
| 31504 | IHVTSKTVK |
| 31505 | IIQGPVKAN |
| 31506 | ILMSMYING |
| 31507 | ILVNQQNGQ |
| 31508 | IMEITNVHE |
| 31509 | IMELTHHNA |
| 31510 | INEMARHPK |
| 31511 | INYTYLWAE |
| 31512 | IPGYAEFCE |
| 31513 | IPGYLEIHR |
| 31514 | IPTYRNRDH |
| 31515 | IQGVLPAMA |
| 31516 | IQMDFMLWR |
| 31517 | IRPTQRPCA |
| 31518 | ISGKSHGDR |
| 31519 | ISQWEHSCW |
| 31520 | ISTDCAEFK |
| 31521 | ITETEAALY |
| 31522 | ITKHHMRNC |
| 31523 | ITSEGFSFT |
| 31524 | IVANGHWLQ |
| 31525 | IVEQCQRMR |
| 31526 | IVFQSLARG |
| 31527 | IVTMGITEI |
| 31528 | IYEIMDYLA |
| 31529 | IYFMFYCSV |
| 31530 | IYPQCALQW |
| 31531 | KATYMEFDT |
| 31532 | KCEEFAHPQ |
| 31533 | KCFLCGSAT |
| 31534 | KCFWAELWA |
| 31535 | KEAVLNHWT |
| 31536 | KHELVTRMY |
| 31537 | KHKPRQSPR |
| 31538 | KHVCLVSWY |
| 31539 | KMEWLAAGP |
| 31540 | KMMAFSEVD |
| 31541 | KNNEPPMFF |
| 31542 | KSYCFMCPT |
| 31543 | KTCEMFTPG |
| 31544 | KTTILEDCC |
| 31545 | KTVEWWGRW |
| 31546 | KVVDCGRYQ |
| 31547 | KWADAEYGH |
| 31548 | KWCYHLFCQ |
| 31549 | LANVWVCME |
| 31550 | LAPEIECRH |
| 31551 | LATSKCSAM |
| 31552 | LATSMCYQA |
| 31553 | LATYGEWHN |
| 31554 | LCDYMHMGS |
| 31555 | LCEENSVDP |
| 31556 | LCFHYMLAQ |
| 31557 | LCWGPLHIS |
| 31558 | LCYFGSRWY |
| 31559 | LCYRTEKWD |
| 31560 | LDSQWMYYN |
| 31561 | LEEAVAAGN |
| 31562 | LESTSFCTY |
| 31563 | LEYDWNGWW |
| 31564 | LFCIVQSRQ |
| 31565 | LGWSNYECA |
| 31566 | LHIRYQSAP |
| 31567 | LHNSYWSLA |
| 31568 | LHWCSQHGH |
| 31569 | LIGFRTMFG |
| 31570 | LILGAKFMG |
| 31571 | LITWDGPTF |
| 31572 | LKFWASEKG |
| 31573 | LKYQFWTYD |
| 31574 | LMMPYMGTH |
| 31575 | LNITLVMGC |
| 31576 | LNPHVSQIW |
| 31577 | LPCKEVCCR |
| 31578 | LPGTPPDNC |
| 31579 | LPKWKEEIY |
| 31580 | LPPMKQDEE |
| 31581 | LQHHKYSSM |
| 31582 | LRCIDPPCQ |
| 31583 | LRNNNCSDI |
| 31584 | LRRWDDFQT |
| 31585 | LRSIEEKLS |
| 31586 | LRSLVAEDF |
| 31587 | LSDRCDVSG |
| 31588 | LSEVMMQPG |
| 31589 | LSHIKVEQD |
| 31590 | LSKNCEYWE |
| 31591 | LSTGNIQFM |
| 31592 | LTKPMPIIL |
| 31593 | LTMISMFSD |
| 31594 | LTRPRVQKL |
| 31595 | LTSRSDFDL |
| 31596 | LVDMCRKGK |
| 31597 | LVGQYWPRD |
| 31598 | LVGSYQHFQ |
| 31599 | LVTRGVSPL |
| 31600 | LWDQTQIAI |
| 31601 | LYAVENQHW |
| 31602 | LYCQQAANW |
| 31603 | LYDHYAAAS |
| 31604 | LYNWMKLIH |
| 31605 | MADLKWEDS |
| 31606 | MAFVSKYIM |
| 31607 | MAHDIPVPV |
| 31608 | MAHQLNHNS |
| 31609 | MATDISGWF |
| 31610 | MAWTHCHKP |
| 31611 | MCLCNMVIQ |
| 31612 | MCSARRQDT |
| 31613 | MCTQPSVKP |
| 31614 | MCYQNIAFP |
| 31615 | MDEQRQHEE |
| 31616 | MDFPSCEMC |
| 31617 | MDHQLCIQY |
| 31618 | MDPILRCLH |
| 31619 | MDPRAHGKV |
| 31620 | MEQWDPDEW |
| 31621 | MFELFQEGW |
| 31622 | MFGGFVKMK |
| 31623 | MFIKWRRYW |
| 31624 | MFMWWNYFP |
| 31625 | MFRISRFMA |
| 31626 | MGILKGEFS |
| 31627 | MGTSCCNYE |
| 31628 | MHLQKHIGC |
| 31629 | MHYLVMRDA |
| 31630 | MIDPKMGDR |
| 31631 | MIHKKDGMY |
| 31632 | MIMQCNCSM |
| 31633 | MKQLSNDGE |
| 31634 | MMDLAYHWQ |
| 31635 | MMEYTHSFE |
| 31636 | MNLPDPSVG |
| 31637 | MNVPWYKDR |
| 31638 | MPCFSVAAA |
| 31639 | MPHPEIRPG |
| 31640 | MQEPMRCAI |
| 31641 | MSFLFFVFM |
| 31642 | MSLLMGHES |
| 31643 | MTEVMREIR |
| 31644 | MTIILPMTS |
| 31645 | MTKPWSIIE |
| 31646 | MTKQTLCHL |
| 31647 | MTQLCEPWL |
| 31648 | MVIQAIWPV |
| 31649 | MVIQDGHEY |
| 31650 | MVMVMEAHN |
| 31651 | MVQWEWCDN |
| 31652 | MYLEKDLSG |
| 31653 | MYQLCHAFA |
| 31654 | NAEFDNNRG |
| 31655 | NAIPWINMD |
| 31656 | NAVAQIVSD |
| 31657 | NCCPGFAIA |
| 31658 | NCPWPTGEE |
| 31659 | NCRMHNAWF |
| 31660 | NDDRCSIEL |
| 31661 | NEQWWVWYR |
| 31662 | NETMMWRAN |
| 31663 | NFHYIGTLG |
| 31664 | NFIFMQDMT |
| 31665 | NFMDYHDMV |
| 31666 | NGMLETDHV |
| 31667 | NIYMPHTCL |
| 31668 | NMATANTCV |
| 31669 | NMDGKNCRT |
| 31670 | NNEMPGWRA |
| 31671 | NNMPMDAEH |
| 31672 | NNMSAHHSK |
| 31673 | NNYHYPHIG |
| 31674 | NNYLELHRY |
| 31675 | NPSSHNQLN |
| 31676 | NQNIWKHNV |
| 31677 | NQRPYDQWA |
| 31678 | NRGCYEMIN |
| 31679 | NRTFKAECH |
| 31680 | NSIFCLENQ |
| 31681 | NSNQDQWRI |
| 31682 | NTCCWKLEN |
| 31683 | NTVIRYRQS |
| 31684 | NVCLEPMDP |
| 31685 | NVEWQVRGP |
| 31686 | NVQWADIGG |
| 31687 | NYKQPVQAP |
| 31688 | NYNITKFFG |
| 31689 | NYWQAQEFY |
| 31690 | PAEVWTHSL |
| 31691 | PCCQEHCQR |
| 31692 | PCNFDNQPH |
| 31693 | PDAAYWFDG |
| 31694 | PDCPYKCYC |
| 31695 | PDGEKCERF |
| 31696 | PDNYIQNHH |
| 31697 | PDQWDFEQY |
| 31698 | PEFMTEMCT |
| 31699 | PFHMALAIE |
| 31700 | PFYHMCAAP |
| 31701 | PGCMIVSQN |
| 31702 | PGELGDENF |
| 31703 | PHNWRSVSW |
| 31704 | PMMQKLCCG |
| 31705 | PNNIGGMKP |
| 31706 | PPGSHKGIN |
| 31707 | PPPDRTFAA |
| 31708 | PQEFREMCP |
| 31709 | PQLKFGQLY |
| 31710 | PQNGMLHYS |
| 31711 | PRAENDESR |
| 31712 | PTITDRFFQ |
| 31713 | PTTNWHYVQ |
| 31714 | PTYHWDSKT |
| 31715 | PVEHVEKEM |
| 31716 | PWHEYAGFT |
| 31717 | PWLAIPRGG |
| 31718 | PWQSGSDGD |
| 31719 | PYASQECIS |
| 31720 | PYPAIELGK |
| 31721 | PYSPHKQSA |
| 31722 | QANVNGHDF |
| 31723 | QCAFIPNSV |
| 31724 | QCEHDRQIM |
| 31725 | QCPVECEHV |
| 31726 | QCYRKVVFR |
| 31727 | QDIWPVCRE |
| 31728 | QEDDFRNKS |
| 31729 | QEKIAMRTN |
| 31730 | QFTSQNEGP |
| 31731 | QFVGRERQV |
| 31732 | QGFYTHTGY |
| 31733 | QHMPRHIWH |
| 31734 | QHWQFCTNA |
| 31735 | QIADQTGIV |
| 31736 | QIGPTISEY |
| 31737 | QIMNYCYFN |
| 31738 | QISCLEHGL |
| 31739 | QISYHIQHH |
| 31740 | QITQHPCEQ |
| 31741 | QITSMKCAN |
| 31742 | QIVQSRSAV |
| 31743 | QIYNWEKMT |
| 31744 | QKGNRTNQI |
| 31745 | QLIVCDTGH |
| 31746 | QMGSRMEMC |
| 31747 | QMHSTFCGW |
| 31748 | QMPIGGCSE |
| 31749 | QNSHKHWDH |
| 31750 | QPCVDWPYF |
| 31751 | QQAPGQMFY |
| 31752 | QQDRLWTTG |
| 31753 | QQKDQAWRH |
| 31754 | QQMIRQLSE |
| 31755 | QQMWGCACC |
| 31756 | QQNVLEHHF |
| 31757 | QQSPVEQCY |
| 31758 | QRQNVQQTW |
| 31759 | QSDAILKYY |
| 31760 | QSDLQHWWY |
| 31761 | QSKELSCMK |
| 31762 | QSKSGQHIA |
| 31763 | QTCTSQDYS |
| 31764 | QTLESMIYH |
| 31765 | QTMFYEIGG |
| 31766 | QTTQTDCTK |
| 31767 | QTVEMSATV |
| 31768 | QVDEWGGHE |
| 31769 | QVDNREGDV |
| 31770 | QVKCMQMWP |
| 31771 | QVVHNEFIH |
| 31772 | QVVSTDAPI |
| 31773 | QVWITAKEE |
| 31774 | QYDAFAVLA |
| 31775 | QYMDQDVAI |
| 31776 | RADEDMTSI |
| 31777 | RADHWGSWP |
| 31778 | RENIHCEGL |
| 31779 | RHELTMSHY |
| 31780 | RHEPLHYFH |
| 31781 | RHWLNGCSN |
| 31782 | RIDINVDCY |
| 31783 | RIELMACTT |
| 31784 | RIKYEHCQL |
| 31785 | RIMYFDKFT |
| 31786 | RITYFDDQC |
| 31787 | RNGFRWTVL |
| 31788 | RPEAYWVPV |
| 31789 | RRDSDSVFK |
| 31790 | RRFCISLKC |
| 31791 | RSAEGISHW |
| 31792 | RSHHHQRMM |
| 31793 | RTIEEINTS |
| 31794 | RTVEIESWV |
| 31795 | RVDDENGGN |
| 31796 | RVDHDMADG |
| 31797 | RVESQACFQ |
| 31798 | RVSVDESDK |
| 31799 | RWEQMAYSW |
| 31800 | SAGVVVQSI |
| 31801 | SATMVADKE |
| 31802 | SAYMALHPL |
| 31803 | SCEWPKQCK |
| 31804 | SCTHWNTWR |
| 31805 | SDAPEHDGQ |
| 31806 | SDLKIETAY |
| 31807 | SDQWYRVSF |
| 31808 | SHMVVTSQG |
| 31809 | SHVESHQWC |
| 31810 | SIDVTSCAF |
| 31811 | SIHPLNQEE |
| 31812 | SIRVYPELS |
| 31813 | SISPIRDMD |
| 31814 | SIVSIVCPN |
| 31815 | SKMAGFPHW |
| 31816 | SKWTCHPKM |
| 31817 | SLKMVDVAV |
| 31818 | SMFKYEARD |
| 31819 | SMHEKQHFE |
| 31820 | SNLDFPNQE |
| 31821 | SNPIMAKGV |
| 31822 | SNPQSVCRG |
| 31823 | SNPRTQTMV |
| 31824 | SNSPHPAVF |
| 31825 | SNVILDFEN |
| 31826 | SNVVKQASD |
| 31827 | SNYRALYGH |
| 31828 | SRDGWPNCI |
| 31829 | SREPIIKGH |
| 31830 | SSGGIWYFD |
| 31831 | SSMEMWTCN |
| 31832 | SSRFAPPPE |
| 31833 | SSRPPSGWV |
| 31834 | SSSQCCTPI |
| 31835 | STHHFSYGE |
| 31836 | SVEAGIKEG |
| 31837 | SVKEEMFSP |
| 31838 | SVLESDICK |
| 31839 | SVNDTVDYF |
| 31840 | SVWDMVKMQ |
| 31841 | SWCPEYWEI |
| 31842 | SWHYLAMQC |
| 31843 | SYHAHQPHC |
| 31844 | SYKQVTAPR |
| 31845 | SYLSFGAPW |
| 31846 | TAAEQKADN |
| 31847 | TAHCTDWQH |
| 31848 | TASMMECAD |
| 31849 | TATTHQRCP |
| 31850 | TCPNVWWHM |
| 31851 | TCVIRNMMI |
| 31852 | TEAPWWSGH |
| 31853 | TEEVDSMAC |
| 31854 | TEGNWDSCW |
| 31855 | TEKAIVYYP |
| 31856 | TFGYMATQV |
| 31857 | TGFHNTFRD |
| 31858 | THCHRHHID |
| 31859 | THIHSGVYG |
| 31860 | THKWDADAK |
| 31861 | TIATVRTCT |
| 31862 | TIDLVYLDK |
| 31863 | TIIPIDWTE |
| 31864 | TILPTCKQA |
| 31865 | TIPCYQRYT |
| 31866 | TISDIPGQP |
| 31867 | TKFMPRGYI |
| 31868 | TLFYHDWYM |
| 31869 | TLSSHWKER |
| 31870 | TMTKRDDFW |
| 31871 | TNTAFMNGT |
| 31872 | TNTSMPAAW |
| 31873 | TNWREMNPR |
| 31874 | TPWFWQETA |
| 31875 | TQTELYCCP |
| 31876 | TRHCHECLG |
| 31877 | TSDEAEHYT |
| 31878 | TSFMCQKFP |
| 31879 | TSGPCMPIK |
| 31880 | TSQHAIWNA |
| 31881 | TSTLVSNNV |
| 31882 | TTEFVRIKG |
| 31883 | TTNDDNCNN |
| 31884 | TTSEKNFDD |
| 31885 | TTTRDEAFM |
| 31886 | TVCTTKSMG |
| 31887 | TVRCMIQLG |
| 31888 | TVSDDWCWI |
| 31889 | TWITTGQFV |
| 31890 | TWMFVHRNN |
| 31891 | TWSDNHAAQ |
| 31892 | TWTNKLCDM |
| 31893 | TYEDHVDRF |
| 31894 | TYIWSMLDR |
| 31895 | TYQNELACY |
| 31896 | VATMAHYGY |
| 31897 | VCIGYDHTP |
| 31898 | VCPLPNQLH |
| 31899 | VCSQTTLWH |
| 31900 | VDKVWWNLI |
| 31901 | VETCRDCEG |
| 31902 | VFGEFDNHS |
| 31903 | VFHFQTKGM |
| 31904 | VFKFDFSYP |
| 31905 | VGDPNFRIQ |
| 31906 | VHAQTIQRE |
| 31907 | VHHTEPERQ |
| 31908 | VHWPQCGHQ |
| 31909 | VIHAPTHKT |
| 31910 | VIPPSIFNN |
| 31911 | VIYHTSYYW |
| 31912 | VKECRRTDQ |
| 31913 | VKSEWYDEQ |
| 31914 | VLGDWSNIC |
| 31915 | VLIPILVAQ |
| 31916 | VLVNVDQLG |
| 31917 | VMPNWKEQT |
| 31918 | VMSCYSRDH |
| 31919 | VMTFRQKAC |
| 31920 | VPGPMIYEF |
| 31921 | VPKTVNRDM |
| 31922 | VQSVRRITG |
| 31923 | VRMICTSSC |
| 31924 | VRVWDYKGT |
| 31925 | VSALQQTQQ |
| 31926 | VSCHWGLAN |
| 31927 | VSRLRQNHW |
| 31928 | VTDHAWTMP |
| 31929 | VTMLMAGMQ |
| 31930 | VTSYWEFQE |
| 31931 | VTYHHHNFL |
| 31932 | VVNFVGAEG |
| 31933 | VVWMNSNHD |
| 31934 | VWFYPQWDS |
| 31935 | VWSCVHADV |
| 31936 | VYWFPDQMM |
| 31937 | WCNGHGSIF |
| 31938 | WDLYLKTMG |
| 31939 | WEAGECDLM |
| 31940 | WEKFWHEYW |
| 31941 | WEWPMTWTT |
| 31942 | WHIFVVIGI |
| 31943 | WIPHCKCPA |
| 31944 | WKAHTEHLG |
| 31945 | WKCWYAPLT |
| 31946 | WLIWGIIEQ |
| 31947 | WMCRNHPFD |
| 31948 | WQMTTRYYM |
| 31949 | WSSTQDQTN |
| 31950 | WTVGDEQPM |
| 31951 | WVEPQTTIY |
| 31952 | WVEPSSKPP |
| 31953 | WWQTFCYGE |
| 31954 | YADMDQAVQ |
| 31955 | YAMSRKQTI |
| 31956 | YATVEEDLQ |
| 31957 | YEHWYEHDD |
| 31958 | YERLFDEQT |
| 31959 | YFRDQEEGQ |
| 31960 | YFSLLMENC |
| 31961 | YHSDYHHNN |
| 31962 | YIEPNHHSG |
| 31963 | YIQRGTDSM |
| 31964 | YLVRQWAIM |
| 31965 | YMSKHEADI |
| 31966 | YNNNHLMMC |
| 31967 | YQQQMTFHN |
| 31968 | YQYFPDNKK |
| 31969 | YRHGSVDGP |
| 31970 | YSCNRLTRF |
| 31971 | YTQLNASQW |
| 31972 | YTTFQGYWA |
| 31973 | YVAEPYYHG |
| 31974 | YVCNICPTP |
| 31975 | YVGQTSSSE |
| 31976 | YVGTIPTFS |
| 31977 | AAEENEAAD |
| 31978 | AAGPDALRP |
| 31979 | AAGTVCLPM |
| 31980 | AAGTVEFGH |
| 31981 | AAGWGGATS |
| 31982 | AAMEVMVKC |
| 31983 | AAMHESCNW |
| 31984 | AAPCCLYYT |
| 31985 | ACAWIENFT |
| 31986 | ACHATTLRY |
| 31987 | ACKQDSELV |
| 31988 | ACMSRAMFN |
| 31989 | ACTRPMVLA |
| 31990 | ACVEHYVAQ |

TABLE 82
Sequences of the 581 to 589 Region in AAV5 VP1
Capsid Polypeptide that Drive Skin Tissue
Tropism

| SEQ | |
|---|---|
| ID | 581-589 |
| NO | Sequence |
| 33991 | NMWYSFVMI |
| 33992 | FWLGLFPAK |
| 33993 | DSQYHVKCG |
| 33994 | AIEWKSTVT |
| 33995 | AIEWRSTVT |
| 33996 | ANAHQAESF |
| 33997 | ANAHQAGSF |
| 33998 | ANLEQSLMA |
| 33999 | ANLEQSLMT |
| 34000 | ANLKQSLMA |
| 34001 | ANTHQAESF |
| 34002 | ATKNRWPIA |
| 34003 | ATKNRWPIV |
| 34004 | CCHGWHNGA |
| 34005 | CCHGWHNGS |
| 34006 | CCHGWYNGS |
| 34007 | CECEKLGTS |
| 34008 | CEGEKLGTS |
| 34009 | CEYEKLGTS |
| 34010 | CFHGWHNGS |
| 34011 | CIALLEQYM |
| 34012 | CNATYQQND |
| 34013 | CSCQWYISV |
| 34014 | CSCRWYISV |
| 34015 | CSYRWYISV |
| 34016 | CYLWCAYQH |
| 34017 | CYLWCDYQH |
| 34018 | CYLWCDYQR |
| 34019 | CYLWCDYRH |
| 34020 | CYLWCVYQH |
| 34021 | CYLWYDYQH |
| 34022 | DHPPVKEVS |
| 34023 | DNLNFNSPT |
| 34024 | DRPPVKEVS |
| 34025 | DTYQNYMFM |
| 34026 | DVHPSNNVQ |
| 34027 | DVHPSNTVQ |
| 34028 | EGPWMFAQG |
| 34029 | EGPWMFTQG |
| 34030 | EYQCWGRFT |
| 34031 | EYQCWGWFT |
| 34032 | GYELQLQMN |
| 34033 | HMYDNELNP |
| 34034 | IAEPQWHKQ |
| 34035 | ITEPQWHKQ |
| 34036 | MCFMAFRFD |
| 34037 | MDYCCEHFP |
| 34038 | MHYCCEHFP |
| 34039 | MLFLKRNIA |
| 34040 | MRFMAFRFD |
| 34041 | MRFMAFRFG |
| 34042 | MRFMAFWFD |
| 34043 | PTPPMQHTA |
| 34044 | QAAHEPQIC |
| 34045 | RHIVHKRTE |
| 34046 | RMYDNELNP |
| 34047 | RRIVHKRTA |
| 34048 | RRIVHKRTE |
| 34049 | RRIVHKRTG |
| 34050 | RSKCPVEIH |
| 34051 | RSKCPVEIR |
| 34052 | TIEWKSTVT |
| 34053 | TNAHQAESF |
| 34054 | TNLEQSLMA |
| 34055 | TTHPMQHTA |
| 34056 | TTPPMQHTA |
| 34057 | WCHGWHNGS |
| 34058 | WPAQGRITT |
| 34059 | YSCRWYISV |
| 34060 | AAVQMSSLS |
| 34061 | ACANTDQRG |
| 34062 | ACMSILEKE |
| 34063 | AEHQNNCAF |
| 34064 | AELPEGWMC |
| 34065 | AGFYEHRSN |
| 34066 | AHGCTNAMS |
| 34067 | AIRISSLVT |
| 34068 | AIVHHGKEP |
| 34069 | AKNYQFQVG |
| 34070 | ALDSCMVSE |
| 34071 | AMIHRNDHC |
| 34072 | ARGMCQDLQ |
| 34073 | ASEVHHAEF |
| 34074 | ASHCMILHT |
| 34075 | ASVDNSGSP |
| 34076 | ASWCLNHMA |
| 34077 | ATSTRQQHA |
| 34078 | AVDPAWHGC |
| 34079 | AVSAFQQDN |
| 34080 | AVSMKAFAI |
| 34081 | CAAHVVLTF |
| 34082 | CAKAKKMTA |
| 34083 | CAQQIYSQT |
| 34084 | CCARIAVAE |
| 34085 | CDTESLAML |
| 34086 | CEHCCITQF |
| 34087 | CEHCCITRF |
| 34088 | CFAFQNMDD |
| 34089 | CFQSNITAN |
| 34090 | CFRSADNWW |
| 34091 | CGAHIMSGH |
| 34092 | CIQWWEMWN |
| 34093 | CKQYIVTDV |
| 34094 | CLNRIQTAS |
| 34095 | CMMPWQSQS |
| 34096 | CMTLILAQE |
| 34097 | CMVWTNVSP |
| 34098 | CNRSIQCMV |
| 34099 | CPGRYWKVG |
| 34100 | CPKLIRQFA |
| 34101 | CPNVWARPW |
| 34102 | CQCRYFDWC |
| 34103 | CSAFTAHQS |
| 34104 | CTFDCNGYW |
| 34105 | CTFSTSEQP |
| 34106 | CTSWVWMQG |
| 34107 | CVAWHAHSA |
| 34108 | CVKQTFTDS |
| 34109 | CVLPLFQET |
| 34110 | CVMAGAIHR |
| 34111 | CVNPMAVAC |
| 34112 | CVQYTRIGR |
| 34113 | CVSPENGMM |
| 34114 | CVWLKQMYE |
| 34115 | CWMNTHIGE |
| 34116 | CWQYQMVTA |
| 34117 | DASPVSRSV |
| 34118 | DFVLRSDPA |
| 34119 | DGTHMDTWL |
| 34120 | DIVGPNTGF |
| 34121 | DKGFKTNSL |
| 34122 | DKHLFDMCR |
| 34123 | DLYTISTRD |
| 34124 | DMFMWNTQE |
| 34125 | DMQCYKRPG |
| 34126 | DMTNCQRCR |
| 34127 | DQRWFNQHA |
| 34128 | DTYLNVMFE |
| 34129 | DVAKKRTAG |
| 34130 | DVCRKGEQF |
| 34131 | DVPLNECDT |
| 34132 | DWRRNTEKQ |
| 34133 | DYAVMKIAM |
| 34134 | EAEEGMGSW |
| 34135 | EARMWHHQW |
| 34136 | ECLESGIQT |
| 34137 | EGWLMMKTG |
| 34138 | EHIHGKFFL |
| 34139 | EIERDTSHE |
| 34140 | EIHTSEMDL |
| 34141 | EIKSWHQVY |
| 34142 | EKNSPWQFA |
| 34143 | ELCWHHGVA |
| 34144 | ENKHHEAFY |
| 34145 | ERMNMIRYA |
| 34146 | ESKIEEAME |
| 34147 | ESVHDNGGH |
| 34148 | ETHHNACSP |
| 34149 | ETHRMTWTT |
| 34150 | ETHTVDADM |
| 34151 | EVNIECCNG |
| 34152 | EVWTWQDAH |
| 34153 | FITGRHSGG |
| 34154 | FPCMSWKQA |
| 34155 | FVFPKHVTL |
| 34156 | FVVPISAYQ |
| 34157 | GACNYMAHM |
| 34158 | GACVMFVGR |
| 34159 | GAQCFIMTP |
| 34160 | GHCACWVAG |
| 34161 | GHFLTTCSF |
| 34162 | GIGFVDMQL |
| 34163 | GIVDCIAAM |
| 34164 | GNDKFLVHN |
| 34165 | GQVATISQF |
| 34166 | GSSQIVKAE |
| 34167 | GTCPYRAIS |
| 34168 | GTFMSSRQS |
| 34169 | GTTAKTEQI |
| 34170 | GTVNFEKMS |
| 34171 | GVNDYRDAG |
| 34172 | GYAMKYLNA |
| 34173 | HAYWCQMFM |
| 34174 | HCDVIVGDW |
| 34175 | HCVPHVGYQ |
| 34176 | HFMCCWFKR |
| 34177 | HHAASATCF |
| 34178 | HIQGMNMWG |
| 34179 | HKMRAIWFA |
| 34180 | HMGMMQCQE |
| 34181 | HNMCPCVDY |
| 34182 | HSGPFCRHG |
| 34183 | HSSPFCRHG |
| 34184 | HSSSKEACF |
| 34185 | HTNWKDGYG |
| 34186 | HTYPAHWLQ |
| 34187 | HWCMLINVE |
| 34188 | IDIDRGHYG |
| 34189 | IFGCIQSAR |
| 34190 | IGFQYGSVE |
| 34191 | IGVSLLNDP |
| 34192 | IIKESVYCQ |
| 34193 | IISMTNRGP |
| 34194 | IMFYTNERV |
| 34195 | IMSKTPSFL |
| 34196 | INVEGGFEY |
| 34197 | IPMEWEHAG |
| 34198 | IRVHDNRQQ |
| 34199 | ISVDCMQRI |
| 34200 | ITAPKWQEK |
| 34201 | ITVEAVHHC |
| 34202 | IWDVRQDMD |
| 34203 | KFDTFIGYQ |
| 34204 | KFWCGHMEE |
| 34205 | KMTSVSWSV |
| 34206 | KNDLEAFSA |
| 34207 | KNGDTMAVG |
| 34208 | KPKCTYPLA |
| 34209 | KTDILDTIV |
| 34210 | KTQEFEVQP |
| 34211 | KVESTTQLC |
| 34212 | KWSYNHFRV |
| 34213 | LAKCGRWSV |
| 34214 | LATLLAQIY |
| 34215 | LCWFLLGVP |
| 34216 | LENCMWGGN |
| 34217 | LFWPDCHFP |
| 34218 | LGLQEWNTP |
| 34219 | LGMPCAWCP |
| 34220 | LIYPWKPGA |
| 34221 | LPEKWNIWK |
| 34222 | LQMNYKAQN |
| 34223 | LSAYLTEAG |
| 34224 | LSIPKEQCW |
| 34225 | LSMWRWESE |
| 34226 | LSNFFGAMQ |
| 34227 | LSSTFPHWH |
| 34228 | LTAKPCTAP |
| 34229 | LTGLQEMAA |
| 34230 | LVQFIYPFE |
| 34231 | LVQFTYPFE |
| 34232 | LWSLRSANF |
| 34233 | LYLMDIAMC |
| 34234 | LYLMDITMC |
| 34235 | LYNLFMHTE |
| 34236 | MAIVDPRSC |
| 34237 | MAQKKCKCP |
| 34238 | MAYCYLMHN |
| 34239 | MDGSYHWDQ |
| 34240 | MFMCYINDN |
| 34241 | MGVEVTQAM |
| 34242 | MIDRHWTAS |
| 34243 | MIRPSINGS |
| 34244 | MITLLPNWP |
| 34245 | MLFKAQAFF |
| 34246 | MLFMAQAFF |
| 34247 | MMCTFDFAG |
| 34248 | MMEIQESCE |
| 34249 | MPEHGMDNW |
| 34250 | MPPWYRHHL |
| 34251 | MQLEANKYH |
| 34252 | MRCIEVKGF |
| 34253 | MSDHLSNGA |
| 34254 | MTHDRAMQC |
| 34255 | MTQCNTEHL |
| 34256 | MTTVHWHSS |
| 34257 | MVLHEMFVY |
| 34258 | MVNPCLLGN |
| 34259 | MVTCAGPTV |
| 34260 | NAGSSGQGA |
| 34261 | NCKYRMELE |
| 34262 | NCVRYKNMD |
| 34263 | NFQMEMDAF |
| 34264 | NFSLTNNGQ |
| 34265 | NILVLCQNN |
| 34266 | NKLDQDNWK |
| 34267 | NKYCFPTSW |
| 34268 | NLMAHGVAP |
| 34269 | NMPNTHCNG |
| 34270 | NQVCWPRIV |
| 34271 | NRLSFMHYG |
| 34272 | NTEFSHLFP |
| 34273 | NVNVNKHNH |
| 34274 | NVNVNKHNR |
| 34275 | NVRDTMRGF |
| 34276 | NVRVEFIQC |
| 34277 | NVVHVGDVK |
| 34278 | PACNCPKTH |
| 34279 | PIWPGVLQH |
| 34280 | PTIKTQFSC |
| 34281 | PTMSLFNEQ |
| 34282 | QAKYVTESA |
| 34283 | QAMKHAFYQ |
| 34284 | QATIWNNRE |
| 34285 | QCCQQPCHN |
| 34286 | QCCQTLFQS |
| 34287 | QCFDNQDEH |
| 34288 | QEAGECKWC |
| 34289 | QHQCYYNQQ |
| 34290 | QIMCNTTGH |
| 34291 | QKSWIVGEK |
| 34292 | QPIFYDRSQ |
| 34293 | QQDGDQQPL |
| 34294 | QQKAISSLP |
| 34295 | QSNNALMGN |
| 34296 | QTFSFAPGY |
| 34297 | QVMDLVYPT |
| 34298 | RAEPFPEEK |
| 34299 | RCEHDKLCL |
| 34300 | RCWFSELHE |
| 34301 | REVYGLFMD |
| 34302 | RGLMCEMMN |
| 34303 | RHMFHEAGW |
| 34304 | RKPPFGPPY |
| 34305 | RLMSGAIVE |
| 34306 | RNKHDMIGC |
| 34307 | RRDCMDSAF |
| 34308 | RVAAPWLPE |
| 34309 | RVILAMGPY |
| 34310 | SAHQFQDYQ |
| 34311 | SAPQFQDYQ |
| 34312 | SDMPHPPLS |
| 34313 | SGCRYNQEQ |
| 34314 | SGLQLMWHH |
| 34315 | SHKFMDLMM |
| 34316 | SHQLIRREN |
| 34317 | SIRYDFYVP |
| 34318 | SLCSMLCTR |
| 34319 | SLCWMHAHH |
| 34320 | SMASVSEIK |
| 34321 | SMEQYWTSN |
| 34322 | SMGVPQAAN |
| 34323 | SMMPKCKAS |
| 34324 | SMQCEVVGP |
| 34325 | SMQCEVVGS |
| 34326 | SMQEYQKHI |
| 34327 | SMYQFADPM |
| 34328 | SQDIESLWP |
| 34329 | SRMQFHNQE |
| 34330 | SRWCPKVPF |
| 34331 | SSGEGKAED |
| 34332 | SSRPKYFCV |
| 34333 | STCHVGKMQ |
| 34334 | STCSIDAQM |
| 34335 | SVCWSNKVP |
| 34336 | SVDRQFVHS |
| 34337 | SVQLWGYQP |
| 34338 | SVRYECSHG |
| 34339 | SYQSCESMQ |
| 34340 | SYSWKQYGV |
| 34341 | TALGIYAHK |
| 34342 | TCIRAMTSC |
| 34343 | TCRPSDWAW |
| 34344 | TCYQNQRSH |
| 34345 | TEWEQNWTP |
| 34346 | TFGTTAIHN |
| 34347 | TGNASNDCW |
| 34348 | THFSKMSDQ |
| 34349 | TKCSWNHIS |
| 34350 | TLCIVPPLI |
| 34351 | TMSTCQQML |
| 34352 | TNQLYRGFW |
| 34353 | TSFMPPSEA |
| 34354 | TSGFFHMYY |
| 34355 | TSHCIPEVE |
| 34356 | TTIGWHVCQ |
| 34357 | TYCKSYRTR |
| 34358 | TYKCWIADN |
| 34359 | VAVDLSWCG |
| 34360 | VFASEWCGS |
| 34361 | VGTKHDVWY |
| 34362 | VKKRDNQST |
| 34363 | VKKRVNQST |
| 34364 | VMKHQHSGR |
| 34365 | VQGYKDSNE |
| 34366 | VSCYTHRQD |
| 34367 | VTFCVHRYQ |
| 34368 | VTTSRWELI |
| 34369 | VYILEGGIH |
| 34370 | WFRITDTCG |
| 34371 | WIITKSTEF |
| 34372 | WMTCFPMQN |
| 34373 | WRDFFNFSH |
| 34374 | WTKMQQNDT |
| 34375 | WVAVLNLNA |
| 34376 | WWQLMNLLP |
| 34377 | YCGIYWNSF |
| 34378 | YEHIDWTFC |
| 34379 | YLHKRCWPS |
| 34380 | YRLLTCASA |
| 34381 | YSALNSYAC |
| 34382 | YSQLFQMTN |
| 34383 | YTFWINNHA |
| 34384 | DTSPVEYAH |
| 34385 | GMVDDKWLY |
| 34386 | QKNVSTKYD |
| 34387 | YCHQEDRIT |
| 34388 | SQETFQKMD |
| 34389 | SRYVNNGTV |
| 34390 | DGDFLQNHH |
| 34391 | ICHTEVMFN |
| 34392 | QTANVKAMY |
| 34393 | SAICQNYDA |
| 34394 | MALTVYWKG |
| 34395 | DTSKTGKHV |
| 34396 | TQCKQLTVM |
| 34397 | KTMGIGGGP |
| 34398 | RVMMGNNQA |
| 34399 | MNSFYRAEW |
| 34400 | AAGHIGSVM |
| 34401 | AAILTDYDE |
| 34402 | AATGFTFFE |
| 34403 | ACDQQQRES |
| 34404 | AHAPQFGPC |
| 34405 | APEIHDYCA |
| 34406 | AYMWEEQAT |
| 34407 | CSMPSVNSD |
| 34408 | CSSCNCHCS |
| 34409 | CTKAYRKPG |
| 34410 | DCMLVPVSG |
| 34411 | DEPFPCDWS |
| 34412 | DGRQVWFFQ |
| 34413 | DHSQNTEPT |
| 34414 | DKFSLPSQD |
| 34415 | DNVTTKKVQ |
| 34416 | DQCPGHISV |
| 34417 | DYCNKKRPV |
| 34418 | EAYANMFHG |
| 34419 | EDEQHLWND |
| 34420 | EEGTVPEVL |
| 34421 | ELPRNYPDP |
| 34422 | EQMPLTNVP |
| 34423 | EQPSVNYLR |
| 34424 | EQTGDFVHH |
| 34425 | ERQARNHYA |
| 34426 | EVNCDWHWQ |
| 34427 | FGPCLMNER |
| 34428 | FGQLIIKHP |
| 34429 | FKMFNWPAY |
| 34430 | FSGVYKQYE |
| 34431 | GDSYYMWKA |
| 34432 | GERGHGERY |
| 34433 | GFHDCSMMI |
| 34434 | GIHEIVVNQ |
| 34435 | GKCIMQMCM |
| 34436 | GLSMVMHFV |
| 34437 | GSMQHRRDC |
| 34438 | GSMRHQRDC |
| 34439 | HGYEHMMSG |
| 34440 | HTERWHMLG |
| 34441 | HTHDWYSAG |
| 34442 | HVYEHMMSG |
| 34443 | IAGISMHNH |
| 34444 | IDDPTMFEV |
| 34445 | IHWHCWPKW |
| 34446 | IIQQYGCST |
| 34447 | IMQPPWHTG |
| 34448 | KEPMPRGFL |
| 34449 | KICHEDISV |
| 34450 | KQALYDTFK |
| 34451 | KQMPLTNVP |
| 34452 | LAYPHERDG |
| 34453 | LHYDTMKNE |
| 34454 | LLANYMVHT |
| 34455 | LPIEQVKWF |
| 34456 | LWSWYDQPS |
| 34457 | MCWSWQDAY |
| 34458 | MQTPQAVMS |
| 34459 | MSMKYLEQT |
| 34460 | MTFQDAATY |
| 34461 | MTIDIDQWC |
| 34462 | MTVIWIWSH |
| 34463 | NNVEQHCQV |
| 34464 | PEYQFECIF |
| 34465 | PGWSVANCS |
| 34466 | PKVNLKGDE |
| 34467 | PQVMWPGWF |
| 34468 | PSTHHTQGM |
| 34469 | PVEELYGTF |
| 34470 | QFFACGVYD |
| 34471 | QPTSHMNMN |
| 34472 | QTIWFIDDN |
| 34473 | RDLMYTDQA |
| 34474 | RIEWEMISD |
| 34475 | SCATAHARE |
| 34476 | SHEKYWYFS |
| 34477 | SKCIMQMCM |
| 34478 | SMFMGRRTE |
| 34479 | SPYSCWMQE |
| 34480 | SRYLYYNYH |
| 34481 | SSYYFMGTP |
| 34482 | SVNRMDQDH |
| 34483 | TCELTMCYL |
| 34484 | THSSCWYQA |
| 34485 | TIRPVMWGH |
| 34486 | TKPPLGCSS |
| 34487 | TQTNKTFMK |
| 34488 | TVCEPKCAA |
| 34489 | TVICIPSGS |
| 34490 | VIWYCFCTL |
| 34491 | VMGIVEPSK |
| 34492 | VMWNQNKTE |
| 34493 | VVHNVKDVH |
| 34494 | WTSILDHSS |
| 34495 | WVASSYKPG |
| 34496 | WYQPRFGRQ |
| 34497 | YDAVWRKYT |
| 34498 | YDEHWNDNS |
| 34499 | YDIMTISPF |
| 34500 | YHKDYMCLG |
| 34501 | YTGRFTCMS |
| 34502 | AATTYNQHA |
| 34503 | ACEQQQRES |
| 34504 | AGDWYIALI |
| 34505 | AGHLQFTCP |
| 34506 | AGVRTQCDN |
| 34507 | ALCSCNQLF |
| 34508 | AMRKIGVWH |
| 34509 | AQGHKCYCS |
| 34510 | AQTNKTFMK |
| 34511 | ASMPVWKHY |
| 34512 | ATIMMHQFG |
| 34513 | AYVEHNASR |
| 34514 | CKTHCPGQY |
| 34515 | CNHQFIDQA |
| 34516 | CSCSYQDMM |
| 34517 | CVNMEMMTA |
| 34518 | CYEQVNSND |
| 34519 | DCADMGYRR |
| 34520 | DCEWMIRLG |
| 34521 | DDEYPAHWD |
| 34522 | DHLPMNQER |
| 34523 | DHQWTDAWV |
| 34524 | DIGYRSTTF |
| 34525 | DRAYRPWND |
| 34526 | DSKSYWMDH |
| 34527 | DVGCLQNMY |
| 34528 | EAYTNMFHG |
| 34529 | ECRMYWFED |
| 34530 | EMMHKMDAG |
| 34531 | ESRQVHVGN |
| 34532 | ESSYVNGIG |
| 34533 | FASYDNTHL |
| 34534 | FGEDGGFKG |
| 34535 | FGPRENNHN |
| 34536 | FHGNYQHDS |
| 34537 | FLCLPQGHI |
| 34538 | FQKVVEWQA |
| 34539 | FQRPNFSLT |
| 34540 | FQSLNKNQS |
| 34541 | FVTQQHSHE |
| 34542 | FWNHHDVAR |
| 34543 | GLCSLNNMP |
| 34544 | GSMQHQRDC |
| 34545 | HEWWGPVSH |
| 34546 | HKELILQPT |
| 34547 | HVQHWMNDR |
| 34548 | IADIDLTSF |
| 34549 | ICQPDMKQS |
| 34550 | IPKYKQFFD |
| 34551 | ISAQCPHVH |
| 34552 | ISHWQQRAE |
| 34553 | ISKAQQHNP |
| 34554 | KHDLTQQRQ |
| 34555 | KHVITGGWW |
| 34556 | KKPWDDHTR |
| 34557 | KLESMAMSM |
| 34558 | KLVCVDAYQ |
| 34559 | KMNTGDNYH |
| 34560 | KQPTPVNGI |
| 34561 | KSARCKLHM |
| 34562 | KSLSGEKNY |
| 34563 | LDMWGDFLL |
| 34564 | LDQSMDIAP |
| 34565 | LQSHSQCFR |
| 34566 | LTWGANAYT |
| 34567 | LVCFADIMQ |
| 34568 | LYRLVAQMN |
| 34569 | MDQLVINSK |
| 34570 | MIKTVAKKE |
| 34571 | MILYFWKSH |
| 34572 | MLAPIDCNT |
| 34573 | MLGPLWKDQ |
| 34574 | MNTRKDPPL |
| 34575 | MTGWSSKQS |
| 34576 | MTYQHVAGA |
| 34577 | MVCGNQRNN |
| 34578 | MVLADNEFI |
| 34579 | NNDRWAIPQ |
| 34580 | NPGWYGLAP |
| 34581 | NQKGHDYSQ |
| 34582 | NQVEVIYQS |
| 34583 | NTSYVTMCT |
| 34584 | PAFVCESPQ |
| 34585 | PKPPLGCSS |
| 34586 | QCEMKHHLA |
| 34587 | QDPHKCRRS |
| 34588 | QPAAKNKWN |
| 34589 | QQNTEVKWE |
| 34590 | QVVVRVQDH |
| 34591 | SCEHCNKPY |
| 34592 | SCHKYTDGN |
| 34593 | SDPDIACVH |
| 34594 | SFCTMQHSV |
| 34595 | SHQWSRQMM |
| 34596 | SNTYAGFQW |
| 34597 | SRMGLELVN |
| 34598 | SSMIMCGYV |
| 34599 | TGTNLHQCQ |
| 34600 | TMHIKRNSH |
| 34601 | TMQCISHLS |
| 34602 | TMRGSNHEP |
| 34603 | TSMKTHFDR |
| 34604 | TSNHENRAD |
| 34605 | TWNEPHQNQ |
| 34606 | VDDQHPTQK |
| 34607 | VIFMPIVHC |
| 34608 | VLVDHPVNE |
| 34609 | WYQSYLNWS |
| 34610 | YAQHEFRLD |
| 34611 | YERQMATPG |
| 34612 | YHPLQSDFG |
| 34613 | YPHHEQHAT |
| 34614 | ATQFYQACI |
| 34615 | GFHDRSMMI |
| 34616 | NCWVNEKCE |
| 34617 | QGSKQMYQA |
| 34618 | RTHGHAKAD |
| 34619 | TSKSISHMD |
| 34620 | YTQFCAAMW |
| 34621 | AQDINRLQA |
| 34622 | IAQSEADYW |
| 34623 | DAVIHTGFA |
| 34624 | DHLNEEQTK |
| 34625 | LHQFEAYYK |
| 34626 | KQQMMIVGN |
| 34627 | YKTSWGQHV |
| 34628 | VDNTDPWIM |
| 34629 | ANIPIEMVC |
| 34630 | TGNHEDPDN |
| 34631 | MMAMKQGFY |
| 34632 | ALTHDDGRT |
| 34633 | AEHCYRHQH |
| 34634 | IEFGVKISG |
| 34635 | KELLCSHDA |
| 34636 | LGDQKLVDE |
| 34637 | HCTETHHTN |
| 34638 | TVGFRFDRA |
| 34639 | GGAEIVSAD |
| 34640 | YIQFEAYAN |
| 34641 | KVHFAKVRI |
| 34642 | LIAYKSHAH |
| 34643 | SPQQPSVFH |
| 34644 | PAEAKQRFD |
| 34645 | MKVSNWVFP |
| 34646 | TIAFNGAVC |
| 34647 | MNTNVMQKD |
| 34648 | PANHARNPK |
| 34649 | YYTKHEQDP |
| 34650 | RQAWPKDGM |
| 34651 | QTAEWYCGA |
| 34652 | THVKRFDYE |
| 34653 | EISICTAKF |
| 34654 | CSECQAIHS |
| 34655 | KERMQVSKA |
| 34656 | FHNAPEHHF |
| 34657 | EPRLYCAQE |
| 34658 | DIEFLDCEN |
| 34659 | DATKEMSWK |
| 34660 | QQAMWLHSS |
| 34661 | CADPQERCD |
| 34662 | TTYPHNTHG |
| 34663 | AVNQWMHSL |
| 34664 | CTNYHVKAG |
| 34665 | WPNIKHQPI |
| 34666 | KNCIMLASN |
| 34667 | PLTIEVNCT |
| 34668 | ATGTYNLQE |
| 34669 | WATWPEPLQ |
| 34670 | YVDHWTLGD |
| 34671 | GLFEGSPGA |
| 34672 | SVLHVQALA |
| 34673 | NGVGRSGDN |
| 34674 | MNLQIGSKG |
| 34675 | NWTGNMQWA |
| 34676 | EVRIQCKID |
| 34677 | SNVACIQAF |
| 34678 | ATMNLSWGL |
| 34679 | QSAHPEPMC |
| 34680 | DHSQNPEPT |
| 34681 | EECPNCEAP |
| 34682 | GWNYMFGSA |
| 34683 | HHTMPIDWN |
| 34684 | GTMPMTGQC |
| 34685 | AQAAAVICW |
| 34686 | DQLAHNAQE |
| 34687 | INMPCPNTK |
| 34688 | RMWMEQYQA |
| 34689 | IMQMHPASG |
| 34690 | IPSHMDAHQ |
| 34691 | HTDSDCRHS |
| 34692 | SYLPRHHKG |
| 34693 | ASDCDGKTW |
| 34694 | QIIMLQHEN |
| 34695 | EICHEDISV |
| 34696 | KTTPAPDQG |
| 34697 | SWYLYYNYH |
| 34698 | LSIRNGMCD |
| 34699 | QFMYCAKGN |
| 34700 | HSSCKNCPW |
| 34701 | DGDYACFKN |
| 34702 | QFLGGPKIQ |
| 34703 | RAVHARQYQ |
| 34704 | EHTQRMDMA |
| 34705 | IVTMQMDSW |
| 34706 | QEINDLKMH |
| 34707 | YTYLHQSSG |
| 34708 | DDRRNTKWE |
| 34709 | VCCSIGPEH |
| 34710 | ALKSFNWCS |
| 34711 | TNDWGEKNA |
| 34712 | CWPEAMIDD |
| 34713 | GAETEPKDS |
| 34714 | CPTYKNKDQ |
| 34715 | HVGAGGMPG |
| 34716 | VTHMTCVEG |
| 34717 | AYMWEGQAT |
| 34718 | KCGNVLCEV |
| 34719 | EHDWRMFHQ |
| 34720 | GPKEHNLCD |
| 34721 | ANSLEGGFE |
| 34722 | YLHERTDKD |
| 34723 | VAEAGYCYK |
| 34724 | KECCFPPCE |
| 34725 | IGEVFDAFS |
| 34726 | AFDGEMSWE |
| 34727 | IEMPTESKH |
| 34728 | YRLHCCNRA |
| 34729 | DAMAYGQFM |
| 34730 | ITELMGCLH |
| 34731 | RGMAHHHLC |
| 34732 | TGGWQRGAF |
| 34733 | FCWSMCEAC |
| 34734 | FCIKDQHVR |
| 34735 | PVTHPQTSK |
| 34736 | RSGVQCCAE |
| 34737 | IWQQCNQKN |
| 34738 | KQSDKWGDD |
| 34739 | MIVHQVSQI |
| 34740 | TSNKKGEAA |
| 34741 | TMFYCHGPN |
| 34742 | LCMNDDRLN |
| 34743 | DCLKKNHSL |
| 34744 | RMDCKWEDL |
| 34745 | TGWNFPVSC |
| 34746 | GSQQMQASQ |
| 34747 | NSKACRGCT |
| 34748 | QCLWVDSNV |
| 34749 | EFCHQHSAI |
| 34750 | HQNLLICNE |
| 34751 | FAGFTPVWM |
| 34752 | NSPHSGVGD |
| 34753 | DKTCYLTCP |
| 34754 | CHGFSGCQT |
| 34755 | NSRLLIKHL |
| 34756 | NCKPDQIAM |
| 34757 | TAMVSECKD |
| 34758 | YDYYKMREG |
| 34759 | SAPEFSVIC |
| 34760 | REAWGIRDS |
| 34761 | KIETRPTCT |
| 34762 | HCTEGYRQT |
| 34763 | MCLNMAKQG |
| 34764 | QNPNCKVMW |
| 34765 | YFHWHDCIG |
| 34766 | KYINFDHHQ |
| 34767 | SFFHQTINP |
| 34768 | KAGYQEGSC |
| 34769 | IACISMHNH |
| 34770 | QPGVRTDDY |
| 34771 | QCQNWRHLS |
| 34772 | LIEMCCSSL |
| 34773 | PTEFRVCPV |
| 34774 | DVCNQSGMS |
| 34775 | RYEISERRC |
| 34776 | NCERWFQIE |
| 34777 | DRREKQLMK |
| 34778 | ENQARCIFE |
| 34779 | ESTHMLYEM |
| 34780 | TWHQGWAEY |
| 34781 | EGGFRDFCE |
| 34782 | AFARQEYAN |
| 34783 | ASSFCKFAS |
| 34784 | ASSRFGSYG |
| 34785 | CHQPKNSDR |
| 34786 | CIAKQIARC |
| 34787 | CMYHEQPGR |
| 34788 | CPQCDVMTS |
| 34789 | CSHETGAMC |
| 34790 | CSNCRQIMG |
| 34791 | CSVCLKYEQ |
| 34792 | CTLKTQAET |
| 34793 | CVKFSHHHA |
| 34794 | DANHCMWWQ |
| 34795 | DCVKSSDGI |
| 34796 | DGCPMTRDL |
| 34797 | DGLFEGHIP |
| 34798 | DIKWQHGAH |
| 34799 | DMYDHEWLM |
| 34800 | DPKCFVWSY |
| 34801 | DWIVMSQKA |
| 34802 | EFNWLLWSG |
| 34803 | EIAHDTSMS |
| 34804 | EIGHKQMAI |
| 34805 | EIVQPQYYA |
| 34806 | EMHKVPSGM |
| 34807 | EVTAVEHIW |
| 34808 | EWTKKTADH |
| 34809 | FLHISSWYQ |
| 34810 | FNMNTNDSG |
| 34811 | GCKKSEWIT |
| 34812 | GGSMLGNGY |
| 34813 | GNKLKNDQM |
| 34814 | HCLAGESYV |
| 34815 | HFHEVAWGS |
| 34816 | HPETMIYGW |
| 34817 | ICNAMLGSE |
| 34818 | IFNHLLPTE |
| 34819 | IRVMFGKHN |
| 34820 | KAIWQGTEN |
| 34821 | KFICTTIAC |
| 34822 | KICPTEYAS |
| 34823 | KIVNQENMK |
| 34824 | KNTMCDNDA |
| 34825 | KSDKVWYVL |
| 34826 | KTQTHTSEP |
| 34827 | KWEQRSKMS |
| 34828 | LMAQWEQTG |
| 34829 | LMAYCYANN |
| 34830 | LMHGVMDHV |
| 34831 | LTQKNLSWQ |
| 34832 | LWPQVMEQR |
| 34833 | MAGHYFNSR |
| 34834 | MAIDNLECK |
| 34835 | MAIISQIEF |
| 34836 | MATISQIEF |
| 34837 | MIHTPDAGC |
| 34838 | MIKFPAKQQ |
| 34839 | MMGSNHCSS |
| 34840 | MNSFYRAEL |
| 34841 | MSPFFWEQG |
| 34842 | MTSTQIRAG |
| 34843 | MYMEQACYD |
| 34844 | NADEKYNNT |
| 34845 | NAPMMACQI |
| 34846 | NGLFEGHIP |
| 34847 | NWDRGDKCI |
| 34848 | QCMVMTEAD |
| 34849 | QDFRMNAGA |
| 34850 | QDFWMNAGA |
| 34851 | QILPHMYAY |
| 34852 | QNNVITVDW |
| 34853 | QPFLHRPCA |
| 34854 | QPFLHRPCV |
| 34855 | QQPRKINLC |
| 34856 | QTMCITIHP |
| 34857 | QWDEFKMRM |
| 34858 | QYQTHPMMH |
| 34859 | QYWWPATHD |
| 34860 | RCEPFNWRE |
| 34861 | REVHVNQNN |
| 34862 | RGSQQMETH |
| 34863 | RKKVEVVVE |
| 34864 | RLKLSAVQR |
| 34865 | RSTWGHIQR |
| 34866 | RVNWEQRMN |
| 34867 | RYQPFSRQW |
| 34868 | SALAHHANF |
| 34869 | SALAHRANF |
| 34870 | SAMCSGSLL |
| 34871 | SANQAHGEF |
| 34872 | SANQRYHDW |
| 34873 | SAVQEERYN |
| 34874 | SAVQKERYN |
| 34875 | SDHQYRGCL |
| 34876 | SMTTWWMFP |
| 34877 | SNSHPFQSH |
| 34878 | SWRWHHWHW |
| 34879 | SYVMNLLGQ |
| 34880 | TAHIMTDVI |
| 34881 | TIDGMMGDH |
| 34882 | TLPKMHMWP |
| 34883 | TQQYTWRDA |
| 34884 | TSYLRKIHS |
| 34885 | TTAGKFAAQ |
| 34886 | TWARYKVGE |
| 34887 | TYDTFGCME |
| 34888 | TYRTRHTDC |
| 34889 | VKHLFYWLP |
| 34890 | VLKSCDRHG |
| 34891 | VVKHGVTTN |
| 34892 | WGATSEFMG |
| 34893 | WHCVAPNSF |
| 34894 | WNDLVRCEE |
| 34895 | WWQPVEYAW |
| 34896 | YHAPMLMCN |
| 34897 | YLAMMMSGD |
| 34898 | YLAMTMSGD |
| 34899 | YQNHYQKKW |
| 34900 | YSVVMRTSY |
| 34901 | YWDACYDQK |
| 34902 | DADCYVPAD |
| 34903 | EGKVPGQFA |
| 34904 | ACDLVKKDQ |
| 34905 | ACEAYQDEK |
| 34906 | AFSQQDYSF |
| 34907 | AGQLNFCSI |
| 34908 | AHNVRCATA |
| 34909 | ALCWKQMDH |
| 34910 | AMCWYYHMQ |
| 34911 | ANVISPQSN |
| 34912 | ASCNTHHEV |
| 34913 | ASGTKYRCN |
| 34914 | ASTMFIDVE |
| 34915 | ATHNVHMFH |
| 34916 | ATMAYRHVN |
| 34917 | AVDCSDHSY |
| 34918 | AVEQWVNHD |
| 34919 | AVVIAQKVV |
| 34920 | AWELVVHQA |
| 34921 | AWNFIRVGN |
| 34922 | CAANVTKMG |
| 34923 | CAAPTFADK |
| 34924 | CAEHLTPSE |
| 34925 | CAGLLGFHW |
| 34926 | CAGVQTTWW |
| 34927 | CAHYFAHAG |
| 34928 | CAKWFDVQI |
| 34929 | CAQSWCSFE |
| 34930 | CCFLINWDR |
| 34931 | CCGWYHEFG |
| 34932 | CCMEVFMYN |
| 34933 | CFTMPVHWG |
| 34934 | CHIQLHAGI |
| 34935 | CIHYKEQYA |
| 34936 | CIHYKEQYE |
| 34937 | CIIHQSKYG |
| 34938 | CIMSRQQDC |
| 34939 | CIRRDDHHH |
| 34940 | CIRWFDWLG |
| 34941 | CIWYTQMGT |
| 34942 | CKETVETVY |
| 34943 | CKMQLFSHG |
| 34944 | CLGWCTACP |
| 34945 | CLTALMKLS |
| 34946 | CLVRYFRDG |
| 34947 | CMTKTSKMS |
| 34948 | CQCSIMSIE |
| 34949 | CRGHIWSMD |
| 34950 | CSACIASAT |
| 34951 | CSLKGPPTH |
| 34952 | CSSSLFSAC |
| 34953 | CSTPITAAV |
| 34954 | CSTPITTAV |
| 34955 | CTCAGVVTC |
| 34956 | CTNLYAMAC |
| 34957 | CTQHMYVKD |
| 34958 | CTWKMMWGG |
| 34959 | CTWTTGEAS |
| 34960 | CVCLAEYVV |
| 34961 | CVFYLPHSP |
| 34962 | CVMSRWNYD |
| 34963 | CVQHFHWDK |
| 34964 | CVRFLNRVA |
| 34965 | CVTRCMARD |
| 34966 | CVVYHNHHT |
| 34967 | CWAQMNHVF |
| 34968 | CWMGIKRQD |
| 34969 | CWMLQHKMM |
| 34970 | CWSINYNYY |
| 34971 | CWTIRHGGP |
| 34972 | CWYLWYNMP |
| 34973 | CYYMLQAAD |
| 34974 | DDDTENDFM |
| 34975 | DGGQTHVSY |
| 34976 | DIHNYLMSE |
| 34977 | DISMNQDVN |
| 34978 | DKRCNKWDP |
| 34979 | DMRLQDHVR |
| 34980 | DNFFTQYLA |
| 34981 | DQESLNAHY |
| 34982 | DREQNEMLG |
| 34983 | DSSSSIGHW |
| 34984 | DTCRRLINE |
| 34985 | DVDWANSLL |
| 34986 | DVRLIGHEA |
| 34987 | DYARCQSVW |
| 34988 | EAEPNGFGL |
| 34989 | EAGDVRRFG |
| 34990 | EAGPHSDTW |

TABLE 83
Sequences of the 581 to 589 Region in AAV5 VP1
Capsid Polypeptide that Drive Spinal
Cord Tissue Tropism

| SEQ | |
|---|---|
| ID | 581-589 |
| NO | Sequence |
| 36438 | GCLQIERRH |
| 36439 | EVTRNHHFN |
| 36440 | CCKPQCKCQ |
| 36441 | MVFLEQHGR |
| 36442 | YITCQYSHH |
| 36443 | ILVPYYTGS |
| 36444 | AWSDLMNCW |
| 36445 | MIEPYTSYN |
| 36446 | NWYTTWIDE |
| 36447 | MMKRNRFIC |
| 36448 | ACVERLHHK |
| 36449 | ECRMAVQSC |
| 36450 | APFFTFTNH |
| 36451 | ASLVKWDFE |
| 36452 | ATMWGDCDY |
| 36453 | AWKMAHNSF |
| 36454 | CHHDVNVPI |
| 36455 | CIMLNGCFQ |
| 36456 | DLCAGNKSQ |
| 36457 | DLCEGNKSQ |
| 36458 | DLCGGNKSQ |
| 36459 | DPMKIFVSH |
| 36460 | EKEWRPLQW |
| 36461 | ESNGMYFDR |
| 36462 | GNCVYHCYE |
| 36463 | HITCQYSHH |
| 36464 | KHMPTLQWQ |
| 36465 | KSTEQMHRA |
| 36466 | KVNCRMIIM |
| 36467 | LNLARYEAP |
| 36468 | MGMWKVWAQ |
| 36469 | MTGDSGKFR |
| 36470 | NIHAVFVQS |
| 36471 | NIHDVFVQS |
| 36472 | NITLVMGQH |
| 36473 | QCCVTYVNS |
| 36474 | QEFYCWIDG |
| 36475 | QNEGRGSIV |
| 36476 | RTAMFESRL |
| 36477 | SHARKVYDC |
| 36478 | SIYQKWVTN |
| 36479 | SKCPWFHNG |
| 36480 | SKNIRNYDI |
| 36481 | SQCVMQFPL |
| 36482 | SYARKVYDC |
| 36483 | TAALGHSFC |
| 36484 | TQGMYVESY |
| 36485 | TTLLVQAEW |
| 36486 | VIIYPQFKC |
| 36487 | YGGMEFEDC |
| 36488 | YGGVEFEDC |
| 36489 | YQTLCMHQD |
| 36490 | YSKVFYFFS |
| 36491 | YTQAMCITA |
| 36492 | ADQHTSPTS |
| 36493 | AIILLCHAE |
| 36494 | ATRCWWREI |
| 36495 | CIIARSTAA |
| 36496 | CNIHWTMAE |
| 36497 | CNNRLDVFN |
| 36498 | CVHERANAN |
| 36499 | DIRPNMFWS |
| 36500 | DNAVNSHVF |
| 36501 | DNYYYWGSL |
| 36502 | EDFVTTSEL |
| 36503 | ETANGMCST |
| 36504 | EVKQGFTQG |
| 36505 | EWHYRNKDA |
| 36506 | GEPDHSPAN |
| 36507 | GIKSCWQNS |
| 36508 | GITEQPLAR |
| 36509 | GSKGMAFCS |
| 36510 | GVNEFLDTN |
| 36511 | HMGYVNNQS |
| 36512 | HQHEAPMDI |
| 36513 | HTTPCARSR |
| 36514 | KSDGQMRQS |
| 36515 | KTYSAMYSP |
| 36516 | LFYMKEDMR |
| 36517 | LPTLRVADK |
| 36518 | MHMSYHWHE |
| 36519 | MHMSYHWRE |
| 36520 | MMLSDDRTP |
| 36521 | NIDQWKHQQ |
| 36522 | QISMAHFLP |
| 36523 | QMEQWHRGG |
| 36524 | QNWAALACP |
| 36525 | QVSVSVIAW |
| 36526 | SITEQPLAR |
| 36527 | SMAICMHDN |
| 36528 | SMQSAEPSC |
| 36529 | STHQSEFTT |
| 36530 | STVYRWDMP |
| 36531 | SVMLKANEV |
| 36532 | SVYHCWRSQ |
| 36533 | TAEVRWPCP |
| 36534 | TASFAQRRS |
| 36535 | TDDPPLLTY |
| 36536 | TFCESMPWV |
| 36537 | TIRYIGMSG |
| 36538 | TKKQMEWQM |
| 36539 | TQQTGWVYN |
| 36540 | TTYIPASAA |
| 36541 | VCHNPDPFT |
| 36542 | VSSDKQVLE |
| 36543 | WECQWDMSM |
| 36544 | WFCYLSPMI |
| 36545 | WFEPSNLIK |
| 36546 | LGMHYKDNC |
| 36547 | ATGTYDLQE |
| 36548 | APKAPKMYH |
| 36549 | DMVTTRSPF |
| 36550 | DQVNKTYVI |
| 36551 | DWSVWIMET |
| 36552 | FLTTVLRVC |
| 36553 | HLTTIPGDR |
| 36554 | IFYECYSNK |
| 36555 | MYIFVWRFM |
| 36556 | PEKVWSDLK |
| 36557 | SWFYMEKMP |
| 36558 | TWLYGWPIK |
| 36559 | VMPIAWQQD |
| 36560 | VQKCHTCGA |
| 36561 | VQKCRTCGA |
| 36562 | WMLLGMTWC |
| 36563 | FTNTKEQAF |
| 36564 | YSWFYWADY |
| 36565 | VRWVRLHKK |
| 36566 | VCYWWLNWF |
| 36567 | THKWFCEDS |
| 36568 | DPYSSKYES |
| 36569 | EDWLRRNIW |
| 36570 | KHYKWGHCQ |
| 36571 | ESVPLRETT |
| 36572 | SSSPGKGWM |
| 36573 | GSTSRNQRD |
| 36574 | AHTQYQGQN |
| 36575 | KFDVIRQAM |
| 36576 | GTIPKQHEF |
| 36577 | QKAIVTQND |
| 36578 | MDYHCQVED |
| 36579 | ERQSTHGQY |
| 36580 | KCWDCHEAG |
| 36581 | NHDEKLWAP |
| 36582 | DGMHRELYS |
| 36583 | AKMWQLENP |
| 36584 | LTNRTQESW |
| 36585 | STALMDANM |
| 36586 | KQAGYDFFH |
| 36587 | SPQQPSVFH |
| 36588 | TTYPHNTHG |
| 36589 | CMEAQGECY |
| 36590 | ISLVWMHYP |
| 36591 | NNGPIWISS |
| 36592 | AHLHNQFMP |
| 36593 | RTEAHLPAP |
| 36594 | EGASPLACT |
| 36595 | DANHCMWWQ |
| 36596 | EKYQIHWDR |
| 36597 | KQEWHVHTE |
| 36598 | KLYGLNHSC |
| 36599 | QSSSSYLWG |
| 36600 | DIRIVSDGK |
| 36601 | MALPLHVLS |
| 36602 | QTMCITIHP |
| 36603 | RHWYIALYA |
| 36604 | LSAKQQFFC |
| 36605 | GEGVEQMDG |
| 36606 | RQIGTGNME |
| 36607 | IMTIIPYGP |
| 36608 | VNDDYECGI |
| 36609 | NTQYSAKND |
| 36610 | SRTICVHGN |
| 36611 | KHIVGQDGG |
| 36612 | KCLPYMEMF |
| 36613 | KAYMMMEGP |
| 36614 | SVTYHEWHP |
| 36615 | YSDMFHKDH |
| 36616 | GNECFHNVE |
| 36617 | IAKIGNCVW |
| 36618 | SGNQIVQAP |
| 36619 | ASFFLNPLE |
| 36620 | DMKQKAEYT |
| 36621 | MMEATHKLS |
| 36622 | GCEEVGRCQ |
| 36623 | CIHPVLVTM |
| 36624 | YDWGRVTLN |
| 36625 | QKAIVAQND |
| 36626 | TFTYEAKFW |
| 36627 | ISWISHASP |
| 36628 | PCQQLMCFM |
| 36629 | VSVQSRAQL |
| 36630 | NVCDNGHEI |
| 36631 | HFQPVFMQP |
| 36632 | HMEQCWCNE |
| 36633 | LPPPEEKWL |
| 36634 | ESAEPRMIP |
| 36635 | ICIRGARAD |
| 36636 | KKHKGWCRA |
| 36637 | AEVPYVMQQ |
| 36638 | MAEQMERDF |
| 36639 | ITASIKDQW |
| 36640 | ETDCMEGGH |
| 36641 | QFAQLWCAK |
| 36642 | KACMSHQGR |
| 36643 | GLFEGSPGA |
| 36644 | AYHCYMSWM |
| 36645 | GQQTIEMSY |
| 36646 | ATGTYNLQE |
| 36647 | GHLFNNNEW |
| 36648 | FAQQIINAS |
| 36649 | AGMYKCQED |
| 36650 | LEIGGKTRT |
| 36651 | NWTGNMQWA |
| 36652 | DIEFLDCEN |
| 36653 | KMAGIGCWY |
| 36654 | QCTAHNTCM |
| 36655 | LHGIINQDN |
| 36656 | AAGREYWLY |
| 36657 | AAVPVNKLT |
| 36658 | ADQSYNEYY |
| 36659 | AELCNNIIK |
| 36660 | AEYTHDCAM |
| 36661 | AGHVFWDGE |
| 36662 | AIGPSWMGV |
| 36663 | AIRPHHAKS |
| 36664 | AQKMEDQSL |
| 36665 | AQKMKDQSL |
| 36666 | ATEMFVQHD |
| 36667 | ATIAQWCIH |
| 36668 | AYQWFMSQP |
| 36669 | CDSIYHHMP |
| 36670 | CHFKYPRIE |
| 36671 | CSYIESWQI |
| 36672 | CTGHFSCQQ |
| 36673 | CTYTMATNR |
| 36674 | DAHKFARYP |
| 36675 | DAKSPCDSF |
| 36676 | DAVNDLTCP |
| 36677 | DDTEMSYCY |
| 36678 | DFWYSMAFP |
| 36679 | DGAWVKMMY |
| 36680 | DQAELLSDH |
| 36681 | DQFRALDLR |
| 36682 | DVASYCQLQ |
| 36683 | EAGMWPSKA |
| 36684 | EAWMVMNDQ |
| 36685 | ECRSNDYDR |
| 36686 | EDVFYQCNH |
| 36687 | EFRRNELGA |
| 36688 | EFWVCTRNH |
| 36689 | EGEARCHWH |
| 36690 | EHFRAYTQY |
| 36691 | EIKCSGGYA |
| 36692 | EKRESFGCG |
| 36693 | ENEMYMKQD |
| 36694 | EQDRGPMQC |
| 36695 | ESMKFRTFS |
| 36696 | FASAFANSD |
| 36697 | FMHSYEKFD |
| 36698 | FNIAESTPM |
| 36699 | FNPNFVTKT |
| 36700 | FNVFIDRSN |
| 36701 | FQRRTQLIT |
| 36702 | FTVFYRLQW |
| 36703 | FTVGGLAFA |
| 36704 | GDEYYDTMG |
| 36705 | GDLSRDQED |
| 36706 | GFRTQDMIE |
| 36707 | GPCMETQHH |
| 36708 | GSEQKIHIN |
| 36709 | GTQSYLVKN |
| 36710 | HATFTWRCL |
| 36711 | HFWITEQMD |
| 36712 | HGCVETNEH |
| 36713 | HMLKMEMFN |
| 36714 | HNDQYEHDE |
| 36715 | HVDNDNMHE |
| 36716 | HVYYCPEAI |
| 36717 | IACPMGIHD |
| 36718 | IAESYRMMH |
| 36719 | IDSWMMIPS |
| 36720 | IGEPQAYMT |
| 36721 | INHGVNHQW |
| 36722 | ISMRSECKD |
| 36723 | ISWITRADI |
| 36724 | IVNLQHYHG |
| 36725 | IVSTSGNND |
| 36726 | IWEDMKGKF |
| 36727 | KAHLVLAGD |
| 36728 | KAMTDAANW |
| 36729 | KCAITETSD |
| 36730 | KFTTAMTEF |
| 36731 | KIVESHGTN |
| 36732 | KKTEEMPVT |
| 36733 | KPCTQAAEP |
| 36734 | KSYSCEQTS |
| 36735 | KTHVMHWEV |
| 36736 | KTYSVMYSP |
| 36737 | KVCNHELFG |
| 36738 | LAAMSLTWW |
| 36739 | LDGVFFEFF |
| 36740 | LFRSHTCHH |
| 36741 | LRTQLLLQD |
| 36742 | LSDSYVRKF |
| 36743 | LVKGREQFN |
| 36744 | LVQCHHVHC |
| 36745 | MAMWARTEP |
| 36746 | MDSQHNKWQ |
| 36747 | MEQFDHCPI |
| 36748 | MFRIEAACP |
| 36749 | MGAHFMNPP |
| 36750 | MKTFWDATF |
| 36751 | MVGPSWDTT |
| 36752 | MYYLMQKTG |
| 36753 | NAMLWRMHN |
| 36754 | NCCQEHNVG |
| 36755 | NEGKWFWAS |
| 36756 | NHDEKLWVP |
| 36757 | NHPVGWDND |
| 36758 | NHVTMCVLR |
| 36759 | NIVDCDDVR |
| 36760 | NLGWQEWDS |
| 36761 | NNPFSRYMP |
| 36762 | NSMWLCTNQ |
| 36763 | NSVMMTMIA |
| 36764 | NSVVMTMIA |
| 36765 | NTIKVEDLG |
| 36766 | NWGDQRRHV |
| 36767 | NYAQYYRAW |
| 36768 | NYCTYHSTH |
| 36769 | PADHKQTSV |
| 36770 | PFCESACHI |
| 36771 | PGDRTISMW |
| 36772 | PGMMYQFAW |
| 36773 | PHVCGEGKI |
| 36774 | PKGWADPCP |
| 36775 | PNWGLDMAP |
| 36776 | PTCIIMKGN |
| 36777 | PTFHRIGSY |
| 36778 | QAARMQKYQ |
| 36779 | QAVSAVDKC |
| 36780 | QDLEWIAGC |
| 36781 | QHHPINCAT |
| 36782 | QHMVTSHNP |
| 36783 | QHNTSYPSF |
| 36784 | QPTLPDEHE |
| 36785 | QTVPPMVQE |
| 36786 | QWIVFTCII |
| 36787 | QWSDIMNCD |
| 36788 | QYAHHYNIE |
| 36789 | QYVDMYTQQ |
| 36790 | RCDTHRSCT |
| 36791 | RDSSNWLCI |
| 36792 | RNYLPLQFD |
| 36793 | RSMLGCHTN |
| 36794 | RTEACTSLP |
| 36795 | RTQWCIADP |
| 36796 | RTYYEENGR |
| 36797 | SATDMTMLT |
| 36798 | SDYEKFLAY |
| 36799 | SEHWHEDAW |
| 36800 | SGNSQNAFM |
| 36801 | SHQHMWHQT |
| 36802 | SIETMDTNA |
| 36803 | SILNAMSLQ |
| 36804 | SNYFNNFGF |
| 36805 | SNYFNNFWF |
| 36806 | SPPNLRAFP |
| 36807 | SPSIDRFWV |
| 36808 | SPYWVMRMG |
| 36809 | SQKVHIDNN |
| 36810 | STHYASTYL |
| 36811 | SVADHYHLK |
| 36812 | SVAMQMHSW |
| 36813 | SVCWIWWTF |
| 36814 | SVKPHYSCW |
| 36815 | SVVYEAIWA |
| 36816 | SWHAEVVNQ |
| 36817 | TAIHMNWRQ |
| 36818 | TAPLVYPHC |
| 36819 | TASDAENYQ |
| 36820 | TCDRSLAEY |
| 36821 | TCDWSLAEY |
| 36822 | TCGYTHIFS |
| 36823 | TCQHNQHVA |
| 36824 | TCSPDEQLD |
| 36825 | TEALAQFMA |
| 36826 | TEKEFMYIC |
| 36827 | THIGSYTQF |
| 36828 | THQYWYGQE |
| 36829 | TIKGQHGMT |
| 36830 | TISDHGAEW |
| 36831 | TLLFAHDYA |
| 36832 | TRATSLTPC |
| 36833 | TTIAQWCVH |
| 36834 | TTNQNSEAM |
| 36835 | TWHNPRNDK |
| 36836 | VCQYCELRG |
| 36837 | VRMVPESEW |
| 36838 | VTNWLSLQY |
| 36839 | VVLPWGLRE |
| 36840 | WKFMFEKWR |
| 36841 | WKTVCPSRQ |
| 36842 | WLAPCAKCG |
| 36843 | WNCSVYTCS |
| 36844 | WSPLYQTVR |
| 36845 | YIHTVENND |
| 36846 | YQNHYQKKW |
| 36847 | IKHIATESR |
| 36848 | VMGTGRMQH |
| 36849 | NWGDQRQHV |
| 36850 | LVFLRDQHY |
| 36851 | CRWTPEIFQ |
| 36852 | KRIKMPPPK |
| 36853 | SWYYDEQSN |
| 36854 | NNYKVQQDG |
| 36855 | HHQLHLKDS |
| 36856 | QDDVLDVTE |
| 36857 | VKNHYEGGP |
| 36858 | KPSQCLPHN |
| 36859 | DYDPDIKDQ |
| 36860 | CDSIYRHMP |
| 36861 | GAMMGNSLP |
| 36862 | CTQGYGDMP |
| 36863 | MIYTGWDMM |
| 36864 | IGKEFIVMP |
| 36865 | TVNTYEHCW |
| 36866 | ECASVDISY |
| 36867 | VNSCRHAWY |
| 36868 | AVFAPNGEV |
| 36869 | KIESRNHYS |
| 36870 | NSQYAHEPP |
| 36871 | AIGHMTQLP |
| 36872 | QIIPQFAYF |
| 36873 | YFMTWGKCD |
| 36874 | IKHGGWGFH |
| 36875 | ECVSKWVVF |
| 36876 | SPPNWRAFP |
| 36877 | AFIQQSNQD |
| 36878 | VSNQFKYND |
| 36879 | QMHRLFNDE |
| 36880 | SKMEEVMDP |
| 36881 | MEQPTCMME |
| 36882 | QPGTQEVLQ |
| 36883 | GCQGIPRMQ |
| 36884 | YSDNLERCG |
| 36885 | YANQAMDYM |
| 36886 | TWPGGCKSV |
| 36887 | AVDDCESER |
| 36888 | KGVWDPSSM |
| 36889 | MTGIVLQNN |
| 36890 | QVNYYTKGM |
| 36891 | QIGSHSFSC |
| 36892 | EINREVRQN |
| 36893 | QTIIGNVWG |
| 36894 | EIGLNGAGT |
| 36895 | RTVQEDGCG |
| 36896 | NCHIDHVQD |
| 36897 | EIKCSVKLE |
| 36898 | WVPTPWRTH |
| 36899 | CTSTMHAFQ |
| 36900 | MSANMMTKG |
| 36901 | LFNYEGRTQ |
| 36902 | CPNLPGMIN |
| 36903 | SAAWQFGGS |
| 36904 | MMATAPELQ |
| 36905 | NYAQYYRTW |
| 36906 | SQDCGRPES |
| 36907 | HEAQKEDCN |
| 36908 | EMDSITQFH |
| 36909 | FHMNSWLSR |
| 36910 | YWIPTHVLN |
| 36911 | WYEMMVGYH |
| 36912 | WDVKFWETM |
| 36913 | SGTSIHTYL |
| 36914 | ECVEWNAPD |
| 36915 | MIEGHAAPR |
| 36916 | PILLCHAPE |
| 36917 | VFLPIARDQ |
| 36918 | FPEASITRW |
| 36919 | PKGVSCPNL |
| 36920 | KHDGEIRVL |
| 36921 | DSDPITVDL |
| 36922 | SDCEKFLAY |
| 36923 | DNVVMWWNQ |
| 36924 | KDFNFEAQG |
| 36925 | PYGQCMRTF |
| 36926 | TNKPKNVLM |
| 36927 | TLGMLLENI |
| 36928 | DVNLQITTH |
| 36929 | ENDYVHGCM |
| 36930 | IDLSHCRCT |
| 36931 | NRTAGSCAC |
| 36932 | LAKPKCKLT |
| 36933 | ATGLPARFP |
| 36934 | NHAWTKPWQ |
| 36935 | CCTQFKCVS |
| 36936 | AHVFEPQRV |
| 36937 | ALFCYNDHV |
| 36938 | AMMPCVGWQ |
| 36939 | AQLCCHNET |
| 36940 | ASCEWSWRL |
| 36941 | AVHWTQKRE |
| 36942 | AVTKCMKTP |
| 36943 | AWAEWCEYS |
| 36944 | AWKDIPSAA |
| 36945 | AWMHAQLSR |
| 36946 | AWMHTQLSR |
| 36947 | AWTEWCEYS |
| 36948 | AYYQTICAA |
| 36949 | CCDLWDDMS |
| 36950 | CCGPSATAQ |
| 36951 | CFCMKEWPN |
| 36952 | CIPPPGYKD |
| 36953 | CKEAWADCT |
| 36954 | CKNVETQMA |
| 36955 | CSAAFQLQP |
| 36956 | CSTKAHRQY |
| 36957 | CTEQIHRHV |
| 36958 | CTQQEASMM |
| 36959 | CVLQENANA |
| 36960 | CVTYLDAWA |
| 36961 | CWGFACLLI |
| 36962 | CWWFACLLI |
| 36963 | DADSSIQLG |
| 36964 | DADSTIQLG |
| 36965 | DAGIGMKFY |
| 36966 | DELWWIANG |
| 36967 | DFAYQCYLN |
| 36968 | DFRTFNQES |
| 36969 | DHEHYTWFG |
| 36970 | DHHTCLEQY |
| 36971 | DKRAWDWLR |
| 36972 | DLMKIDKWD |
| 36973 | DMHMLNCCE |
| 36974 | DMHMLNCCG |
| 36975 | DPLKHCYDN |
| 36976 | DRHNHDVAI |
| 36977 | DRHNHDVTI |
| 36978 | DSRQQMSLE |
| 36979 | DTEYQTNAN |
| 36980 | DVAHKDDML |
| 36981 | DVQQANGRY |
| 36982 | DVTMAEVIG |
| 36983 | DVWTLPECS |
| 36984 | DYEVCGNWS |
| 36985 | DYEYVMSIK |
| 36986 | DYNESDMQN |
| 36987 | EEMLVTRAS |
| 36988 | EHETVWHNP |
| 36989 | EHNIYNNGL |
| 36990 | EIMQPMQGM |
| 36991 | ELNCLSRYV |
| 36992 | ENGPVRRIG |
| 36993 | ENQRMRQIN |
| 36994 | ERTDTNLWS |
| 36995 | ERTDTNVWS |
| 36996 | ESGSTRNDG |
| 36997 | ESYEHQMHR |
| 36998 | ETHRTSNNC |
| 36999 | ETHYPKCQE |
| 37000 | ETHYPKYQE |
| 37001 | ETKFRLFPK |
| 37002 | EVMIAAALP |
| 37003 | EVMIAAALS |
| 37004 | EVVGYPIIC |
| 37005 | EWDDWYSQS |
| 37006 | FAQLKWMMS |
| 37007 | FICQIPETK |
| 37008 | FWHCINACP |
| 37009 | FWPCINACP |
| 37010 | GAPRYHESR |
| 37011 | GATRQEHAF |
| 37012 | GATWQEHAF |
| 37013 | GELWWIANG |
| 37014 | GFVQKVNDT |
| 37015 | GICWPNWYH |
| 37016 | GQNIVEMSW |
| 37017 | GVYKTNQPQ |
| 37018 | GYEGFEIVD |
| 37019 | GYEGSEIVD |
| 37020 | GYEYVMSIK |
| 37021 | HCSPDDMKC |
| 37022 | HDFPFWSKG |
| 37023 | HIHFDEWNC |
| 37024 | HKGFLQEWI |
| 37025 | HPAGYEKWW |
| 37026 | HQKSEWPNT |
| 37027 | HSDRDEIEE |
| 37028 | HSDRDEIEG |
| 37029 | HVLHQDFFI |
| 37030 | HVLHQDFFT |
| 37031 | HWNTGMLTN |
| 37032 | HWYVVLRDW |
| 37033 | IAKDMHEMS |
| 37034 | IAVLHSSQR |
| 37035 | IFALIPDAN |
| 37036 | ILDNDQVTY |
| 37037 | ILQCHEAIG |
| 37038 | IMLMCTPRK |
| 37039 | IPGFKHSDW |
| 37040 | ISTTFQWYS |
| 37041 | ITGAVQPFL |
| 37042 | KGGACDACI |
| 37043 | KKGWEFMYG |
| 37044 | KKGWEVMYG |
| 37045 | KVDPIRHGQ |
| 37046 | KVPTDGANS |
| 37047 | KYVRWFQAW |
| 37048 | LAESHYPFE |
| 37049 | LDTKWDQHI |
| 37050 | LDTKWDQHV |
| 37051 | LGGMHPCNN |
| 37052 | LGGMHPYNN |
| 37053 | LINKFPQQE |
| 37054 | LISKFPQQE |
| 37055 | LMEMTLRMG |
| 37056 | LNNHMKKYP |
| 37057 | LRISKWMTG |
| 37058 | LVCDSGYAA |
| 37059 | LVRIWHETM |
| 37060 | LWECYGTHG |
| 37061 | MAFMQGLGW |
| 37062 | MDGQACLNQ |
| 37063 | MDNQEDRCE |
| 37064 | MFALIPDAN |
| 37065 | MHAQRMKYP |
| 37066 | MHTQRMKYP |
| 37067 | MLMYLGHLT |
| 37068 | MLMYLGHRT |
| 37069 | MNEDCMQTS |
| 37070 | MNHEHGPIG |
| 37071 | MPRHKHSQT |
| 37072 | MSDDVVLAL |
| 37073 | MTVQNVPEM |
| 37074 | MVDGPAASG |
| 37075 | MYYVTQMCN |
| 37076 | NAWMYVNMS |
| 37077 | NAYPWHLFC |
| 37078 | NAYSWHLFC |
| 37079 | NIIKCVECC |
| 37080 | NIIKCVEGC |
| 37081 | NLRIHAHRT |
| 37082 | NPMRPSVEQ |
| 37083 | NVPIRALQK |
| 37084 | PIALYANCS |
| 37085 | PIELYANCS |
| 37086 | PIGLYANCS |
| 37087 | PLTTDCSPN |
| 37088 | QGFADGRPT |
| 37089 | QHLEINCVS |
| 37090 | QLVYNMWDC |
| 37091 | QMCMNISHY |
| 37092 | QMCPSCCVT |
| 37093 | QPDYEEPPS |
| 37094 | QPWVPSILH |
| 37095 | QVETVPYLS |
| 37096 | QYGHKQFEY |
| 37097 | QYRVDSYPF |
| 37098 | RCQDKVMPY |
| 37099 | RCSHDDMKC |
| 37100 | RCSPDVMKC |
| 37101 | RFDMWWLFI |
| 37102 | RHKDTEWFI |
| 37103 | RIEMFWCNN |
| 37104 | RIEMFWCTN |
| 37105 | RIKMFWCNN |
| 37106 | RLDCNQFWE |
| 37107 | SALTEPPFP |
| 37108 | SAPTELPFP |
| 37109 | SCGQFEWDN |
| 37110 | SGSWEAFGF |
| 37111 | SGSWEDFGF |
| 37112 | SGSWEGFGF |
| 37113 | SHVFEPQRV |
| 37114 | SIEMFWCNN |
| 37115 | SMHTWQTAK |
| 37116 | SMHTWQTTK |
| 37117 | SNERRHMCA |
| 37118 | SPMGVAMNG |
| 37119 | SSIPWNYYT |
| 37120 | SVTYLDAWA |
| 37121 | TCAGFCAPE |
| 37122 | TCLHDYGYG |
| 37123 | TLFCYNDHV |
| 37124 | TNAYQYHVG |
| 37125 | TPEHGLMVG |
| 37126 | TQCEIFEAH |
| 37127 | TRKVAEEMW |
| 37128 | TRPVEMQPG |
| 37129 | TVEPEYFQA |
| 37130 | TVEPEYFQD |
| 37131 | TVEPEYFRD |
| 37132 | TVFMNAHWA |
| 37133 | TVNEGEYGP |
| 37134 | TWSYDPASS |
| 37135 | VAEPSWQNN |
| 37136 | VCTLNNHTM |
| 37137 | VGNETHLGD |
| 37138 | VGNKTHLGD |
| 37139 | VHITVCRYG |
| 37140 | VIYCHPMVP |
| 37141 | VMDWWLDEN |
| 37142 | VPHTTCHTH |
| 37143 | VPHTTYHTH |
| 37144 | VQKWGLMQQ |
| 37145 | VSGSTWNDG |
| 37146 | VTEFIMHCG |
| 37147 | VTEVIMHCG |
| 37148 | VTHTVHQET |
| 37149 | VVVVNGVTE |
| 37150 | VYQRFCGKR |
| 37151 | WINPTEMGA |
| 37152 | WIVLSDTIG |
| 37153 | WKHCWMLMQ |
| 37154 | WLRILRAPH |
| 37155 | WLRILRAPY |
| 37156 | WSQEQITFD |
| 37157 | YNGKWPRLT |
| 37158 | YNMCSAGSE |
| 37159 | YNMYSAGSE |
| 37160 | YSGCWWQHG |
| 37161 | YSNRIPIGF |
| 37162 | YVPPNYTEQ |
| 37163 | LHWPITVQP |
| 37164 | VLNKNWTMC |
| 37165 | ISDCMDNYW |
| 37166 | YCVNMQYDV |
| 37167 | NSEREYAIR |
| 37168 | RAQSHMEDY |
| 37169 | EKFSANKFQ |
| 37170 | RSGQGEGHM |
| 37171 | GSHQLMWIG |
| 37172 | YMWGTELGC |
| 37173 | VTNLYEGSG |
| 37174 | EMWHHLSTP |
| 37175 | WWRATFGQA |
| 37176 | NWQWIAVQD |
| 37177 | HNLYGVYWG |
| 37178 | NQSANWLQN |
| 37179 | TGGQNYAHE |
| 37180 | CSEPDPAWN |
| 37181 | AFSSERVYA |
| 37182 | CCILWESGT |
| 37183 | CPQCDVMTS |
| 37184 | QSSFWPAPT |
| 37185 | SKWHFYLRQ |
| 37186 | VRFTRTSLQ |
| 37187 | KAEMKMGCN |
| 37188 | MANHSISAD |
| 37189 | IDRCCWDYA |
| 37190 | EPGLLWHTQ |
| 37191 | HTHDVRVHK |
| 37192 | WKPAAYSNC |
| 37193 | CGHTACSQC |
| 37194 | DICLTHDHC |
| 37195 | REIRDDYIY |
| 37196 | VYTPKYQDL |
| 37197 | KCESWKDFF |
| 37198 | QNEPEKQVK |
| 37199 | LASYESVEY |
| 37200 | HMHFDEWNC |
| 37201 | KPFHVEMSL |
| 37202 | GRHYIAAGY |
| 37203 | TTMTHSTLD |
| 37204 | AEICHKRED |
| 37205 | DAAEELRNA |
| 37206 | LIAHDHKCF |
| 37207 | YTKIESVHL |
| 37208 | ALMMDDITP |
| 37209 | TQSHWNKMS |
| 37210 | KTFQYMGPT |
| 37211 | SNHESMHMD |
| 37212 | SCGQFEWGN |
| 37213 | VEPYMFAPM |
| 37214 | TTETRNWVL |
| 37215 | TAQQFAHSG |
| 37216 | EANPYTATK |
| 37217 | FQGMCQLRE |
| 37218 | IIIQRSRTY |
| 37219 | DFTEEGEAY |
| 37220 | MVKLDAQDA |
| 37221 | EIMQPLQGM |
| 37222 | VPNRNCQFS |
| 37223 | CIHSVHFAA |
| 37224 | MFAKWMSGS |
| 37225 | ESESWIHMP |
| 37226 | SALTELPFP |
| 37227 | FGLQMMYIV |
| 37228 | PAPMSEYNF |
| 37229 | ECALAQYGS |
| 37230 | SHMGKHHHM |
| 37231 | ITAKQEVTS |
| 37232 | AADCGSFDH |
| 37233 | AADCGSFYH |
| 37234 | ADHTENFSD |
| 37235 | AEDCGSFDH |
| 37236 | AEDTPYNIY |
| 37237 | AESPQRWAL |
| 37238 | AEWSEGECA |
| 37239 | AEWSEWECA |
| 37240 | AEWSEWEGA |
| 37241 | AGQKHWHYS |
| 37242 | AIDHHTEVV |
| 37243 | ATFTFNEDN |
| 37244 | AVCMEEREP |
| 37245 | AWCWQVILM |
| 37246 | CAPKQECSR |
| 37247 | CDVWWFPNL |
| 37248 | CNRQKYTAK |
| 37249 | CNSSESHYY |
| 37250 | CWTMLMFST |
| 37251 | CYQHLNTMN |
| 37252 | DANHCMWWR |
| 37253 | DDHCCTTPT |
| 37254 | DEPTLEEDM |
| 37255 | DGTLAILCT |
| 37256 | DGTLAIMCA |
| 37257 | DGTLAIMCT |
| 37258 | DGYQKYQEQ |
| 37259 | DIACLMNST |
| 37260 | DIEGYGHSW |
| 37261 | DISQTTITK |
| 37262 | DKLPMAMDR |
| 37263 | DKNVFEEHV |
| 37264 | DNCQVENHL |
| 37265 | DPPHRNCTI |
| 37266 | DQEKCLSFG |
| 37267 | EARMPDDWS |
| 37268 | EARMPNDWP |
| 37269 | EFGFKIHRY |
| 37270 | EIFHACNSG |
| 37271 | ELQYYKEHT |
| 37272 | ELYGLNHSC |
| 37273 | EMGIRGIWG |
| 37274 | ESCHLQHEE |
| 37275 | ESCHPQHGE |
| 37276 | EVCTDDGNV |
| 37277 | EYATKVKMD |
| 37278 | FHCAMEGRT |
| 37279 | FSRHWECVQ |
| 37280 | FSRRWECVQ |
| 37281 | FWSNIIGIY |
| 37282 | GFNMNNMVA |
| 37283 | GHLDPFDCG |
| 37284 | GLCIYDHRH |
| 37285 | GMSMSQSHK |
| 37286 | GNECFHNVA |
| 37287 | GNHTFNQDQ |
| 37288 | GQNLNRGVC |
| 37289 | GTAGPCRSS |
| 37290 | GWCWQVILM |
| 37291 | GYNKWGRNE |
| 37292 | HEGLIMRLA |
| 37293 | HIGNKHIEC |
| 37294 | HMEQCRCNE |
| 37295 | HMYATEHCS |
| 37296 | IACRTHQTE |
| 37297 | IARRTHQTE |
| 37298 | ICHCCMDWG |
| 37299 | ICIRGARAA |
| 37300 | ICIRGVRAD |
| 37301 | ICTFYQPSP |
| 37302 | IHSPFMPYH |
| 37303 | ITTARNQYN |
| 37304 | IVSPFQTGT |
| 37305 | IYRLCQDSD |
| 37306 | KAEVKSPAP |
| 37307 | KATMTNCCF |
| 37308 | KCEGEEAGR |
| 37309 | KEDFGWKHM |
| 37310 | KKEQDHRDK |
| 37311 | KQAGYGFFH |
| 37312 | KYEVPGKYG |
| 37313 | LGSPFYERG |
| 37314 | LHSYQWPLG |
| 37315 | LKVVQAYEC |
| 37316 | LNNRTQESW |
| 37317 | LQLIVVSQT |
| 37318 | LQYPTACGC |
| 37319 | LSAKRQFFC |
| 37320 | LTNRTQESR |
| 37321 | MAIQVFMYD |
| 37322 | MAIQVIMSD |
| 37323 | MGTILQEVA |
| 37324 | MHAEDVTSV |
| 37325 | MISAPPDTY |
| 37326 | MKRGTAFCA |
| 37327 | MKYQDTHTF |
| 37328 | MTIQVFMSD |
| 37329 | MVASVAAEE |
| 37330 | MYMHHALSP |
| 37331 | NGKTNPCQK |
| 37332 | NGTQCLNMG |
| 37333 | NGTQCPNMD |
| 37334 | NISWIQDYM |
| 37335 | NMMTLWSHL |
| 37336 | NPGWRYMWC |
| 37337 | NQDEKLWAP |
| 37338 | NSEKVYGMF |
| 37339 | NSNHYNAMR |
| 37340 | NTEPTLVTR |
| 37341 | NTEYSAKND |
| 37342 | NVDAHEQYR |
| 37343 | NVDRQHAQG |
| 37344 | NVRNGTNTC |
| 37345 | NVYVTPVVC |
| 37346 | NYRCFDPMH |
| 37347 | PMMIIPYVA |
| 37348 | PNGHHWMIP |
| 37349 | QAPVIPIMT |
| 37350 | QHNSCKDAW |
| 37351 | QMNCLKLGP |
| 37352 | QNIATMRGF |
| 37353 | QNICLGVPT |
| 37354 | QSFPCMFKD |
| 37355 | QSPVPNRYA |
| 37356 | QTFSEGWRL |
| 37357 | QVNKGAPQE |
| 37358 | RADWSDIAD |
| 37359 | RCNLYLEYS |
| 37360 | RDGDANCSL |
| 37361 | REKWGHMHT |
| 37362 | REQHIAGAD |
| 37363 | RGLVQDCCR |
| 37364 | RHVHRYWEC |
| 37365 | RVKAYAPTT |
| 37366 | RWSYMQADK |
| 37367 | SAMYGSTWA |
| 37368 | SAVYTAACN |
| 37369 | SGMTWNFHA |
| 37370 | SHIQLNAWW |
| 37371 | SIFMEVAWC |
| 37372 | SIQRNCAFW |
| 37373 | SLMRFLPSI |
| 37374 | SMGWAILEG |
| 37375 | SMPVGTEID |
| 37376 | SSASVNHTN |
| 37377 | SSDMQVCWT |
| 37378 | SSDNSHQGR |
| 37379 | SSKFHERQH |
| 37380 | STDNTGTAC |
| 37381 | STGSCAPLY |
| 37382 | TAVKSEAST |
| 37383 | TDHTENFSD |
| 37384 | TFANPPENA |
| 37385 | THGIYGTHD |
| 37386 | TISWLDTEV |
| 37387 | TISWPDTEV |
| 37388 | TKDAWNGLC |
| 37389 | TMSNYGCPQ |
| 37390 | TQIMRVISV |
| 37391 | TTQRDGQWY |
| 37392 | TVGPAGISE |
| 37393 | VDGLQCCCP |
| 37394 | VDIMRNDTG |
| 37395 | VEFRPKISR |
| 37396 | VGIMWNDTG |
| 37397 | VNWFVRAYI |
| 37398 | VSYWFQWGS |
| 37399 | VTATSPGIE |
| 37400 | VVTKPAYFV |
| 37401 | WADLWQYQW |
| 37402 | WATPPLQRC |
| 37403 | WRIRPQQEI |
| 37404 | WRMFESAPG |
| 37405 | YAQPDWVMA |
| 37406 | YCMWYATPP |
| 37407 | YHVPYQHLP |
| 37408 | YHWHINSFD |
| 37409 | YPELGKCEI |
| 37410 | YTGHTNNGR |
| 37411 | YTGLMWVWA |
| 37412 | AMVLFLVDN |
| 37413 | CALKQECSR |
| 37414 | CHNQMWQIC |
| 37415 | CISQTFRYY |
| 37416 | CWAEFRMRV |
| 37417 | CWMLGRDCC |
| 37418 | DGAAPLTFC |
| 37419 | DIAMNPRMC |
| 37420 | DITLWMATE |
| 37421 | DLMWHVNAI |
| 37422 | EMCHSMGDF |
| 37423 | EMCVMSFGY |
| 37424 | FFGWKKSNY |
| 37425 | FGREGCPTE |
| 37426 | FVELHCHTE |
| 37427 | GDTVMLVPS |
| 37428 | GGPFKVASS |
| 37429 | GTFHNTATI |
| 37430 | HTRVVESTG |
| 37431 | IAKIGNCVC |
| 37432 | IHTGTNSYA |
| 37433 | IMVHCREMG |
| 37434 | IPQVCQFGQ |
| 37435 | IRSQTLTDC |
| 37436 | IVHKLESVN |
| 37437 | KEARSQTGD |

TABLE 84
Sequences of the 581 to 589 Region in
AAV5 VP1 Capsid Polypeptide that Drive
Spleen Tissue Tropism

| SEQ ID | 581-589 |
|---|---|
| NO | Sequence |
| 39438 | CTTYYCWQE |
| 39439 | TMMYYESGQ |
| 39440 | YNMVHGDEP |
| 39441 | WGSLEWDRA |
| 39442 | DSGTKKRMH |
| 39443 | QHELEGNRF |
| 39444 | TEESLVRTY |
| 39445 | DDYYQLHES |
| 39446 | LLEHPEKDL |
| 39447 | LWQHTCIHG |
| 39448 | SDFTLGYHH |
| 39449 | MIEQWDACS |
| 39450 | PQYDCWKGS |
| 39451 | NHYPDQEAE |
| 39452 | EQPHCKWTA |
| 39453 | SHNNNPSGE |
| 39454 | EVADIVSLA |
| 39455 | LVELSIKQQ |
| 39456 | LWEHTFDCD |
| 39457 | VQSFENQAC |
| 39458 | VFRVKCAAD |
| 39459 | QSDWISIPC |
| 39460 | RANTWTRRH |
| 39461 | KVPSQLVFE |
| 39462 | ENCMSRMKP |
| 39463 | RLTEDMPDM |
| 39464 | NNGVHIFCE |
| 39465 | SHMSYGQVQ |
| 39466 | DVCDDHKPN |
| 39467 | GDNMVQWVS |
| 39468 | GYASIVKRD |
| 39469 | DNYMQLNCV |
| 39470 | DVAPMHQVE |
| 39471 | QLRCEYQHT |
| 39472 | EASIQASWN |
| 39473 | PCAMHNWGC |
| 39474 | MNANETHGA |
| 39475 | CECHWAYPS |
| 39476 | GFQRTHVDQ |
| 39477 | MEPHYWEWP |
| 39478 | RYMNAAKFF |
| 39479 | MNEYIMSMH |
| 39480 | IYMSTGNTP |
| 39481 | QQTKNNSTD |
| 39482 | EIYSQLTDL |
| 39483 | VTKLAENVY |
| 39484 | WDYSMTKKH |
| 39485 | FKHSLREWT |
| 39486 | ALAKFQCWS |
| 39487 | FCDLAHHYE |
| 39488 | PWTTPENYT |
| 39489 | NLAPMIQVC |
| 39490 | EAELENVNM |
| 39491 | YMALDLIPY |
| 39492 | TTKNAISFE |
| 39493 | STLMPDEMA |
| 39494 | LSAQRLVCD |
| 39495 | EPMPMPVWN |
| 39496 | HYVFGMSST |
| 39497 | GHNVTAMAI |
| 39498 | LWSRFISMD |
| 39499 | VPTPWMGIS |
| 39500 | IAYHYDQQN |
| 39501 | IEHCSNRYA |
| 39502 | FAQQIINAS |
| 39503 | YVLYSWRTS |
| 39504 | VTNWLSLQS |
| 39505 | QMVTQLNWE |
| 39506 | IQNPAMWGT |
| 39507 | EFAHEWYVN |
| 39508 | PYGQCMRTF |
| 39509 | INYISFVDR |
| 39510 | GVNDLQNIC |
| 39511 | CSKATWGAE |
| 39512 | DYVDQCQVS |
| 39513 | MCGCGWAKG |
| 39514 | NAELQQADI |
| 39515 | ESSQEIKTC |
| 39516 | AIRQFMMHL |
| 39517 | QTQICHSWM |
| 39518 | WFDRSWHIL |
| 39519 | FRAVYYYHS |
| 39520 | TFHQSDYWE |
| 39521 | CGCQVKCHT |
| 39522 | WNWLKVMHE |
| 39523 | MASQGGQVR |
| 39524 | DVEFCMKQY |
| 39525 | INMPVSKDQ |
| 39526 | QMCVWMTRQ |
| 39527 | SSEYSQFAI |
| 39528 | DSIIKDQSD |
| 39529 | EHPEWKCMY |
| 39530 | WKATPGGQC |
| 39531 | STARHNMQL |
| 39532 | RYQSHCWLH |
| 39533 | TFAKSDWLA |
| 39534 | NSQLSMTFG |
| 39535 | RFDQFWDTY |
| 39536 | QTSHQDQDC |
| 39537 | DHELTRSEA |
| 39538 | MITNNCNIS |
| 39539 | FNESGWPTW |
| 39540 | MWGCKLFVC |
| 39541 | SKGWADLCP |
| 39542 | HIHDTMSDY |
| 39543 | WHTIMTYES |
| 39544 | SFVHMFQGH |
| 39545 | WVHPYSYCA |
| 39546 | QVSFNARYA |
| 39547 | FSTSYCHED |
| 39548 | WLIGMWNTG |
| 39549 | NSTMEHSRW |
| 39550 | KYEFLLKYI |
| 39551 | HCDSPAATP |
| 39552 | DIEFLDCEN |
| 39553 | YSDPKNMSI |
| 39554 | ACIPQMHSA |
| 39555 | NCMHLQGCQ |
| 39556 | SRVMIHVYV |
| 39557 | SMFTHQLAQ |
| 39558 | DVTPTSWIE |
| 39559 | WCEAVGDYA |
| 39560 | GDMYYWKLQ |
| 39561 | GWDMLSVAK |
| 39562 | NMEPLKLQA |
| 39563 | CWECCTDSG |
| 39564 | GNECFHNVE |
| 39565 | LSTMFPEQT |
| 39566 | TAFQSLQQM |
| 39567 | NGVGRSGDN |
| 39568 | QAWPKTEYI |
| 39569 | YNNDMKQRR |
| 39570 | MLHTSECVQ |
| 39571 | GSHWYDCNQ |
| 39572 | PTNYGWMWR |
| 39573 | AHEDPNTWC |
| 39574 | QSMVACYTI |
| 39575 | PWRWHKSAC |
| 39576 | WINEANCLM |
| 39577 | LSPWPQTDQ |
| 39578 | LETMMRHGE |
| 39579 | LGSPFYECG |
| 39580 | ACQFYISRP |
| 39581 | VFKPLDKRY |
| 39582 | TFVRKPSLM |
| 39583 | NDMQMNWVH |
| 39584 | HAKQANGMV |
| 39585 | VKFRITTEC |
| 39586 | CVEQYVEQG |
| 39587 | MQPMESATW |
| 39588 | VEDRVFACC |
| 39589 | EVRIQCKID |
| 39590 | KOKTQNRNA |
| 39591 | KVAEGQNML |
| 39592 | YPTMTFPEE |
| 39593 | ASKQGTHDY |
| 39594 | YVIGQKDEM |
| 39595 | PHASGYTCI |
| 39596 | EKRLEGWVY |
| 39597 | NLSNLVSCR |
| 39598 | YTELMKKAC |
| 39599 | QCTAHNTCM |
| 39600 | TWAPWCCYF |
| 39601 | ICHIRTRGN |
| 39602 | CIQPTWDSY |
| 39603 | EQELKNSTQ |
| 39604 | NGYLVHACQ |
| 39605 | NADMSFRNR |
| 39606 | DQRHGNVSP |
| 39607 | TWYMNWMWH |
| 39608 | KGNPLDGDT |
| 39609 | VTMIHNAFH |
| 39610 | SFKSVSQYN |
| 39611 | AFGLTTGHT |
| 39612 | CVLSRSTMR |
| 39613 | LCKFANGAA |
| 39614 | QSYPTMYQC |
| 39615 | QPYWVAVDP |
| 39616 | CKYKTKSIR |
| 39617 | QCTLEISYV |
| 39618 | EICMRSGFI |
| 39619 | WVDQNVAQD |
| 39620 | YYNHKIVEP |
| 39621 | QHEQSNHIW |
| 39622 | YVDHWTLGD |
| 39623 | NIQLLHSYC |
| 39624 | IMNPTAQNN |
| 39625 | ATMNLSWGL |
| 39626 | HTNYTVGYE |
| 39627 | PWMHTMLNP |
| 39628 | EAGPRKRDV |
| 39629 | LEIGGKTRT |
| 39630 | ARWFSTVEQ |
| 39631 | KSDAQRNVA |
| 39632 | WERCLELNQ |
| 39633 | RCADNPYFR |
| 39634 | QTDILRDVQ |
| 39635 | MDGRDEIIH |
| 39636 | ASDMQFWST |
| 39637 | CSIQINNCR |
| 39638 | PIWQGYWPW |
| 39639 | GAANRIQSW |
| 39640 | SGDWDFGEW |
| 39641 | TQCKQLTVM |
| 39642 | MHGIARMQQ |
| 39643 | LMKTSPSYW |
| 39644 | KMHGNERCF |
| 39645 | RYPCCQSPF |
| 39646 | AVYLDHVFS |
| 39647 | ITAARNQYN |
| 39648 | VGNVMIKMC |
| 39649 | HHFHVNLDI |
| 39650 | NRIQMTDFQ |
| 39651 | QISKPSVQC |
| 39652 | DVMQYQNHM |
| 39653 | MQHTDWTSG |
| 39654 | CWILHHRCS |
| 39655 | FSWPIHEPD |
| 39656 | RDQCRMESG |
| 39657 | QIKTHSNVF |
| 39658 | DSAKQWHHQ |
| 39659 | SVLHVQALA |
| 39660 | ETNDNMILH |
| 39661 | FQWRTEMNN |
| 39662 | FQTCDFWAT |
| 39663 | TVNWYSGFG |
| 39664 | YNFDYMKDG |
| 39665 | KCADLIIRS |
| 39666 | CCLWSYWAF |
| 39667 | FQTLILSGL |
| 39668 | ILINDREWG |
| 39669 | EPEGELDKT |
| 39670 | MYAGWLNHG |
| 39671 | FLTCDQFEW |
| 39672 | NMFQLKPQN |
| 39673 | PYGPIEYDE |
| 39674 | GGDGRVHNK |
| 39675 | VDRLFRMEY |
| 39676 | QWFTKGVGE |
| 39677 | QSRCVDNTV |
| 39678 | QIIYRCRST |
| 39679 | KDGRNPHCL |
| 39680 | AIGPALCLT |
| 39681 | TQPEDHWLA |
| 39682 | TGMTFNSFR |
| 39683 | NAIPYFVQA |
| 39684 | DVKSWAVCD |
| 39685 | VLPMGPVDP |
| 39686 | CDHVTKHPT |
| 39687 | SNVACIQAF |
| 39688 | THVKRFDYE |
| 39689 | VFNLMLQDK |
| 39690 | ECGCMYWGY |
| 39691 | MYTGMRCWR |
| 39692 | MDNCPPMWN |
| 39693 | ATMWMQWWD |
| 39694 | TNSCECVDR |
| 39695 | GHLFNNNEW |
| 39696 | ASMHEETCL |
| 39697 | VCDGHTAFP |
| 39698 | TISQIPWVR |
| 39699 | KNCIMLASN |
| 39700 | QGKQLYHMC |
| 39701 | YFPSIQCYE |
| 39702 | KMAGIGCWY |
| 39703 | GGALARSSI |
| 39704 | PDLWYEKSS |
| 39705 | NVWTNIAEM |
| 39706 | HIKVEHEEV |
| 39707 | EHVQCSADM |
| 39708 | LYMMALLPT |
| 39709 | ASIATMDRI |
| 39710 | YKGMEKNPV |
| 39711 | QMKGATEFC |
| 39712 | AADIFAIND |
| 39713 | NSGVLIGME |
| 39714 | TSNHNMALE |
| 39715 | LIEKQRWML |
| 39716 | SGFFVHTSN |
| 39717 | QSSHCKQMV |
| 39718 | RDYIERREP |
| 39719 | RQAWPKDGM |
| 39720 | QHHWKWSTE |
| 39721 | YCECGHWPN |
| 39722 | HMGIAYNDH |
| 39723 | LCAVCGDCD |
| 39724 | TPLMMCQAS |
| 39725 | TYESLQNSW |
| 39726 | GRALGKKQG |
| 39727 | YTKYNWKAA |
| 39728 | TATCGIEVE |
| 39729 | MPWLDKPPC |
| 39730 | TRQWMCVHC |
| 39731 | DQEAQVVTG |
| 39732 | MVENGLRLQ |
| 39733 | EAGCKMWPT |
| 39734 | AIGCFQNGG |
| 39735 | GLFEGSPGA |
| 39736 | YLNKNPHQM |
| 39737 | CQMLCRPPV |
| 39738 | VNLPYGYAK |
| 39739 | LMTPQVKQA |
| 39740 | SQERMDYDG |
| 39741 | NTHTGNDRL |
| 39742 | QGHKTMHVF |
| 39743 | FGGMTPYGV |
| 39744 | SECEVLCSL |
| 39745 | EIHMFGQSE |
| 39746 | QAKMILDGT |
| 39747 | VSDECYFDM |
| 39748 | REWMFTLMA |
| 39749 | CNGYWMCMS |
| 39750 | QIRSLITHD |
| 39751 | QFAQLWCAK |
| 39752 | RYNNCPNLW |
| 39753 | EVAFTTSSD |
| 39754 | YVKMCSMRN |
| 39755 | HTVMEWSPT |
| 39756 | TFMFNSEWS |
| 39757 | AGMYKCQED |
| 39758 | FCILASDMA |
| 39759 | DLIVATEWF |
| 39760 | QGFSTDVKP |
| 39761 | FMIMAKTQA |
| 39762 | QSAHPEPMC |
| 39763 | YDYWMQAPT |
| 39764 | VVCRMWHQP |
| 39765 | GSKIIMHTE |
| 39766 | NHSLCWDSK |
| 39767 | TSSKHLVFD |
| 39768 | TKMIIFDSW |
| 39769 | ENTMWQSSQ |
| 39770 | PVICVWHDN |
| 39771 | NWTGNMQWA |
| 39772 | MNLQIGSKG |
| 39773 | DRKQADQIY |
| 39774 | GGNAGRDTT |
| 39775 | WGGMPNNQG |
| 39776 | HSMMPGWPS |
| 39777 | DSDTWAMHE |
| 39778 | EGIMNHAPT |
| 39779 | FQVMDHANP |
| 39780 | NSNSMNCVS |
| 39781 | QQVLGLAGN |
| 39782 | QSSPDQRAL |
| 39783 | AAPIAHSMR |
| 39784 | ADPTTETPH |
| 39785 | AFNEMEQGD |
| 39786 | AHAPWCWAV |
| 39787 | AMTFSVSCS |
| 39788 | ANYALHYDR |
| 39789 | AREFWHCRD |
| 39790 | ATNIKESVN |
| 39791 | AVHAFPWSF |
| 39792 | AVKDMKKWD |
| 39793 | CDEDLYVTP |
| 39794 | CHIQVDHHF |
| 39795 | CMDGQLKWF |
| 39796 | CPWNSQVTL |
| 39797 | CQQYAAWAV |
| 39798 | CRIASFKAI |
| 39799 | CSAYCHACE |
| 39800 | CSSDRDGLP |
| 39801 | DDGHHVPIM |
| 39802 | DDMFAQYFS |
| 39803 | DHSMWTWAE |
| 39804 | DICNATCWV |
| 39805 | DITQTDMWG |
| 39806 | DIWTAKENV |
| 39807 | DLAMTMVNP |
| 39808 | DSGPQTQMV |
| 39809 | DTAINQCWI |
| 39810 | DVEPSECSW |
| 39811 | DVQNANQMF |
| 39812 | DVRYELPYR |
| 39813 | EAVGMPLCN |
| 39814 | EDWYWMNLC |
| 39815 | EFKVYQNCH |
| 39816 | EKYTFCIFL |
| 39817 | ELLYTLMFH |
| 39818 | EMEAQLLGE |
| 39819 | EPCLVNISH |
| 39820 | EQNASMTMH |
| 39821 | ERDHIPCYA |
| 39822 | ESSNTQPWH |
| 39823 | ETASDPQTI |
| 39824 | ETKYSYEVG |
| 39825 | ETTDGYGQV |
| 39826 | ETVWNDEQS |
| 39827 | EVALQMFSQ |
| 39828 | EVCQNINEA |
| 39829 | EVMPITGYG |
| 39830 | FHELRHVDT |
| 39831 | FKDWNTVGM |
| 39832 | FKTRETTEI |
| 39833 | FMHHTVRSF |
| 39834 | FMPAKSVAD |
| 39835 | FMRSVQWNA |
| 39836 | FSGMETRPP |
| 39837 | FTLVATNIH |
| 39838 | FVSSVTQVA |
| 39839 | GAGDMPMLD |
| 39840 | GDQGPRQQL |
| 39841 | GFHPIPWFP |
| 39842 | GIEMDLCNG |
| 39843 | GIGPQLNWA |
| 39844 | GIRFFEKGI |
| 39845 | GMHMPCDKN |
| 39846 | GQAKYWMGT |
| 39847 | GYKPDRLHD |
| 39848 | HMVINQMDM |
| 39849 | HPRQGWMHS |
| 39850 | IAGYRHNTD |
| 39851 | IFHNYDLTG |
| 39852 | INDARQHFG |
| 39853 | INVAMQKEG |
| 39854 | IRGDEMLED |
| 39855 | ISNYVGFHG |
| 39856 | IVLHMSEGY |
| 39857 | KAMPEQLWP |
| 39858 | KAVSYQNPF |
| 39859 | KMHRRIWGA |
| 39860 | KNTETMYSE |
| 39861 | KNWPMDKHL |
| 39862 | KVNEMVAEV |
| 39863 | KVTEYCDCG |
| 39864 | LIICTWKAG |
| 39865 | LIMEAMLFS |
| 39866 | LLRRNDELK |
| 39867 | LMDCHSVIP |
| 39868 | LMQMDKPNG |
| 39869 | LPHMCKCKG |
| 39870 | LTMADGLMR |
| 39871 | LTNEVREEN |
| 39872 | LTWISHGVN |
| 39873 | MAMEYIEMY |
| 39874 | MARIYPGTC |
| 39875 | MAYMEHHVG |
| 39876 | MEMEYEEFM |
| 39877 | MKQDSTQGM |
| 39878 | MPLCGYNVR |
| 39879 | MTDQPPSKV |
| 39880 | MVWNVAMMD |
| 39881 | NDELCVYCW |
| 39882 | NGVEQIKHE |
| 39883 | NMVMFVMDN |
| 39884 | NQICHQYDA |
| 39885 | NSTVHWQQN |
| 39886 | NTQWYMMNA |
| 39887 | NVEDNRKVF |
| 39888 | NYRNQKNCH |
| 39889 | QRATTKRHA |
| 39890 | QVHPVNHWG |
| 39891 | QVQVVDEWS |
| 39892 | QYQHERGWL |
| 39893 | RGGYIVAGI |
| 39894 | RGYWDVREG |
| 39895 | SAECMTVSS |
| 39896 | SIWHYQHMQ |
| 39897 | SIYKYTALN |
| 39898 | SMLLSNQNQ |
| 39899 | SNQCLPYDC |
| 39900 | SSAVIMKFP |
| 39901 | STHTVSWDM |
| 39902 | STQPSAKQV |
| 39903 | SVDYVEGYC |
| 39904 | TAMAYTSCE |
| 39905 | TCQHAPSPM |
| 39906 | TFEQIPCWT |
| 39907 | TFHVCQTMG |
| 39908 | TMQVMEVIS |
| 39909 | TNLRPMTER |
| 39910 | TNNTMRHEF |
| 39911 | TRFSSQGKM |
| 39912 | TSHAQTQMD |
| 39913 | TSRTRNCTH |
| 39914 | TYALDMVST |
| 39915 | TYGLYDDSY |
| 39916 | VAMYYESEM |
| 39917 | VDNKFMEHK |
| 39918 | VDPCANEGH |
| 39919 | VIEEMTMVC |
| 39920 | VIMPMQPRC |
| 39921 | VMLHLIHYP |
| 39922 | VPCIWVLKD |
| 39923 | VSTCCQANG |
| 39924 | VVECSGRAN |
| 39925 | VWVVRNHAD |
| 39926 | WCAGATMQW |
| 39927 | WEKLSQRNR |
| 39928 | WEMNDMQDY |
| 39929 | WINIPCHCI |
| 39930 | WKCIVPGQD |
| 39931 | YMVWSMMMI |
| 39932 | AAAMCGYQV |
| 39933 | AAAPSDDEP |
| 39934 | AADREKSWM |
| 39935 | AADTQSYIR |
| 39936 | AADTYVNHM |
| 39937 | AADYDDQQW |
| 39938 | AAEEGHTWS |
| 39939 | AAFQPDVNF |
| 39940 | AAHEKQCAA |
| 39941 | AAHTWHVAW |
| 39942 | AAHYNDGCT |
| 39943 | AAISMLWVE |
| 39944 | AAKEATSEG |
| 39945 | AAKWKAKCA |
| 39946 | AANPIESVD |
| 39947 | AAPLKRGLP |
| 39948 | AASHTEGFL |
| 39949 | AASPCNDFW |
| 39950 | AASQCFCMP |
| 39951 | AATFKSRHA |
| 39952 | AAWHANLSR |
| 39953 | ACDVKKHFG |
| 39954 | ACHAYPSAW |
| 39955 | ACMEAIMTT |
| 39956 | ACTDFMSAC |
| 39957 | ACVEYIDSA |
| 39958 | ADAVQLMCP |
| 39959 | ADCDYGRKF |
| 39960 | ADCIFESDG |
| 39961 | ADMYANENE |
| 39962 | ADPKYVPTT |
| 39963 | ADQCNPLCA |
| 39964 | ADQCTMLRF |
| 39965 | AEMCKTLIT |
| 39966 | AEPCKLKHF |
| 39967 | AEPRHDKAC |
| 39968 | AFKGHYMAA |
| 39969 | AFNSGMAKI |
| 39970 | AFVENMWNF |
| 39971 | AGALPQAWA |
| 39972 | AGCFFTGIK |
| 39973 | AGDHFLLVG |
| 39974 | AGRMRFAKM |
| 39975 | AGSYYGVIN |
| 39976 | AHCVPWSWT |
| 39977 | AHEATSCMV |
| 39978 | AHGCGFAWP |
| 39979 | AHKQMDWHT |
| 39980 | AHLPLMLGT |
| 39981 | AHQGVAGTY |
| 39982 | AHVDNNLLF |
| 39983 | AIAAENDCT |
| 39984 | AIAEHWEGR |
| 39985 | AIAEYAQQG |
| 39986 | AIAWYMGEA |
| 39987 | AIECSWGWI |
| 39988 | AIKMDGDFQ |
| 39989 | AILPMLMQS |
| 39990 | AINIMDQEA |
| 39991 | AIPDYQIYE |
| 39992 | AISHNMKTP |
| 39993 | AISSCARQI |
| 39994 | AITMSMPTW |
| 39995 | AITVGCGWA |
| 39996 | AIVEYWSSC |
| 39997 | AIYYCLSDW |
| 39998 | AKCTWRSYH |
| 39999 | AKEDQHQFQ |
| 40000 | AKEYMMLSY |
| 40001 | AKLKQAWSG |
| 40002 | AKMDWKLPM |
| 40003 | AKQQPEKQY |
| 40004 | AKSCWPSVW |
| 40005 | AKTHNPNTD |
| 40006 | ALCSRDSSN |
| 40007 | ALLAMQLAT |
| 40008 | AMLHTMGHW |
| 40009 | AMMNWQMMN |
| 40010 | AMMTVEYFN |
| 40011 | AMREWRVFP |
| 40012 | AMTSMIQSG |
| 40013 | AMYGKAAGD |
| 40014 | AMYNPYTQK |
| 40015 | ANCQPSRVC |
| 40016 | ANHTKLKMY |
| 40017 | ANMENWVQS |
| 40018 | ANMSATYYE |
| 40019 | APAPNVRYS |
| 40020 | APNMMNWIA |
| 40021 | APVIGWGAY |
| 40022 | AQDNNNCAA |
| 40023 | AQEPVQGNY |
| 40024 | AQHVAIFNL |
| 40025 | AQMYRWVMT |
| 40026 | AQPLEHHYN |
| 40027 | AQQEVKCGY |
| 40028 | AQSHGTEMF |
| 40029 | AQTWGGAGS |
| 40030 | ARDHGKWNW |
| 40031 | ARDLYHRSE |
| 40032 | ARITWNVCT |
| 40033 | ARKQIVGMD |
| 40034 | ARQHMRRQP |
| 40035 | ASADVHYAQ |
| 40036 | ASANRKQMD |
| 40037 | ASAWQMMMP |
| 40038 | ASCEMTQTN |
| 40039 | ASDQFMADE |
| 40040 | ASHKDGWAT |
| 40041 | ASHNFSDEG |
| 40042 | ASNMHANAH |
| 40043 | ASRDYDLAP |
| 40044 | ASSSCNGGH |
| 40045 | ASTLGCDCC |
| 40046 | ASVTRVAVL |
| 40047 | ASVVSDECN |
| 40048 | ATAFGGEYY |
| 40049 | ATCYLMKLD |
| 40050 | ATDDECCVG |
| 40051 | ATDDVRCLW |
| 40052 | ATDGYSKWD |
| 40053 | ATDPERSQW |
| 40054 | ATDTWDRAS |
| 40055 | ATEYQPNTW |
| 40056 | ATFSCDPWN |
| 40057 | ATGEEDVNW |
| 40058 | ATGFGTEDS |
| 40059 | ATHEMKGMN |
| 40060 | ATIQHEAGM |
| 40061 | ATKFFRMET |
| 40062 | ATLTWNYSQ |
| 40063 | ATNFDVGHE |
| 40064 | ATNSNYEHR |
| 40065 | ATNWMRDQT |
| 40066 | ATSEQRIVV |
| 40067 | ATSLAHCVC |
| 40068 | ATTDNYHDR |
| 40069 | ATTHETVLY |
| 40070 | ATTNSSGSH |
| 40071 | AVAMLVDVW |
| 40072 | AVAWKPRMP |
| 40073 | AVCNDDHSA |
| 40074 | AVCRPSTSY |
| 40075 | AVDCYGLFS |
| 40076 | AVELETQWW |
| 40077 | AVFYPYITD |
| 40078 | AVGHNGSHR |
| 40079 | AVLNSWPAC |
| 40080 | AVMPYCELE |
| 40081 | AVQDKVCTC |
| 40082 | AVVLTDWNE |
| 40083 | AWAFHHAVP |
| 40084 | AWANYIRHE |
| 40085 | AWEFVNYPP |
| 40086 | AWEVNGHYH |
| 40087 | AWWFEAPMV |
| 40088 | AYAPEPMCP |
| 40089 | AYLNFYDGP |
| 40090 | AYLQFQIQG |
| 40091 | AYMTSNSFG |
| 40092 | AYSRATTHI |
| 40093 | AYVAKKNMN |
| 40094 | CANEHNHTA |
| 40095 | CAPVSTRKA |
| 40096 | CAQSIPEAW |
| 40097 | CATCHTLHM |
| 40098 | CCCEENYRR |
| 40099 | CCEQFELGS |
| 40100 | CCLPGWGSL |
| 40101 | CCNLSDEHH |
| 40102 | CCRSGDCMP |
| 40103 | CCTVTQAPE |
| 40104 | CCYHTQLLH |
| 40105 | CDMALVGGD |
| 40106 | CDNNWPIKQ |
| 40107 | CDWSNIYDV |
| 40108 | CDYLLAQCC |
| 40109 | CEDFCMVNR |
| 40110 | CEFRPVVDH |
| 40111 | CEHTGSFQS |
| 40112 | CFDRSIGYE |
| 40113 | CFHYLMNTW |
| 40114 | CFMDGWPFV |
| 40115 | CGAICDAVV |
| 40116 | CGEPPHIQD |
| 40117 | CGRHWDPWF |
| 40118 | CGTGMNGAF |
| 40119 | CHTLKSGEH |
| 40120 | CHYPYCVPL |
| 40121 | CIGFAMESG |
| 40122 | CIQRYTTEQ |
| 40123 | CIRVQEFGG |
| 40124 | CIVEEMAHW |
| 40125 | CIYEQERKM |
| 40126 | CKNGPAYVP |
| 40127 | CLFYDEAMG |
| 40128 | CLHVPHNSL |
| 40129 | CLLVAECLQ |
| 40130 | CLQFKCRLS |
| 40131 | CLQHTSMKC |
| 40132 | CLSDNYHNQ |
| 40133 | CLVSIKDCH |
| 40134 | CMACTDIYM |
| 40135 | CMGNEDMST |
| 40136 | CMIEKNMDY |
| 40137 | CMPLSMGTR |
| 40138 | CMQNSIKEC |
| 40139 | CMRDCNHYG |
| 40140 | CNASKFWQD |
| 40141 | CNQQIQFFG |
| 40142 | CNRQGCMHE |
| 40143 | CNVYRFHEA |
| 40144 | CPCHEIHPQ |
| 40145 | CPFFHNKLA |
| 40146 | CPQCLPVTE |
| 40147 | CPYCARQHE |
| 40148 | CPYQDVFQH |
| 40149 | CQFEYQIFQ |
| 40150 | CQHLVRQGL |
| 40151 | CQLSDSFLF |
| 40152 | CQMGFCCHS |
| 40153 | CQQFYERVD |
| 40154 | CQQGSPNDW |
| 40155 | CQVESIGPH |
| 40156 | CRCWSQGHQ |
| 40157 | CREDKLEAR |
| 40158 | CRIPMPARC |
| 40159 | CRVWEPLRM |
| 40160 | CSEMMQIHC |
| 40161 | CSESGLWCS |
| 40162 | CSKGDCQPH |
| 40163 | CSLLFVSCV |
| 40164 | CSMYWDWLF |
| 40165 | CSTHIAQSE |
| 40166 | CSTSFSQRG |
| 40167 | CSYNDMDRC |
| 40168 | CTCMTKCVD |
| 40169 | CTELVQFIG |
| 40170 | CTFSIACDH |
| 40171 | CTGIPKCVK |
| 40172 | CTNQHKLSS |
| 40173 | CVCQYHQTN |
| 40174 | CVDPGVSWP |
| 40175 | CVIHMKTKI |
| 40176 | CVKQYMFAQ |
| 40177 | CVMDAMAIP |
| 40178 | CVMTEDFQF |
| 40179 | CVSESNIWH |
| 40180 | CVYRHQREY |
| 40181 | CWDSTLYGY |
| 40182 | CYEPYHRHV |
| 40183 | CYFFARNEM |
| 40184 | CYGHRQIKD |
| 40185 | CYKLSNYIV |
| 40186 | CYQWHHDGT |
| 40187 | DACKTINQF |
| 40188 | DACLYWAPW |
| 40189 | DADAWQMVT |
| 40190 | DAGMPWFMN |
| 40191 | DAGPSVVVQ |
| 40192 | DAKCGRMVA |
| 40193 | DAMPSCEFS |
| 40194 | DAQLLVMRM |
| 40195 | DAQWPLQRD |
| 40196 | DARFTFVES |
| 40197 | DCAEWLVRD |
| 40198 | DCGRAQMMA |
| 40199 | DCHGAFDRE |
| 40200 | DCSLMGSDP |
| 40201 | DCSRHHMAQ |
| 40202 | DCSTORTIW |
| 40203 | DCWSEQQPM |
| 40204 | DCYHIDMGC |
| 40205 | DDAPLKITA |
| 40206 | DDDKKHACR |
| 40207 | DDGQCDRKG |
| 40208 | DEHCDSPAI |
| 40209 | DEMKWLNCS |
| 40210 | DFCKQCNGL |
| 40211 | DFDWEVNPT |
| 40212 | DFEPNEEHY |
| 40213 | DFLVFSVQG |
| 40214 | DFSENGHTF |
| 40215 | DFSERRIVV |
| 40216 | DFSQFNQGH |
| 40217 | DFTLSFDAH |
| 40218 | DGPLTCEQA |
| 40219 | DGPMSKQEL |
| 40220 | DGQCPNAWV |
| 40221 | DGTHNHLQG |
| 40222 | DGTYLMFHA |
| 40223 | DGVDKEWYE |
| 40224 | DHQANRTCC |
| 40225 | DHTWQHEGH |
| 40226 | DICAQSPGA |
| 40227 | DIDSVQEVP |
| 40228 | DIDVGHSFT |
| 40229 | DIFLNKCTY |
| 40230 | DIGTRVCWH |
| 40231 | DIHNDIQHG |
| 40232 | DILPVQAPG |
| 40233 | DIMPYASQG |
| 40234 | DIQIQTYQS |
| 40235 | DIQLYNNAF |
| 40236 | DISAMAGWY |
| 40237 | DIWQASTRA |
| 40238 | DKESWCEVT |
| 40239 | DKHTYEHGM |
| 40240 | DKRCFQQSN |
| 40241 | DKSSTARGM |
| 40242 | DKYDTDWAF |
| 40243 | DKYSNKCLR |
| 40244 | DLEGTEQSL |
| 40245 | DLEYIWRQD |
| 40246 | DLIMGRKKQ |
| 40247 | DLMLNFHLD |
| 40248 | DLQHFMSYP |
| 40249 | DLTQRISMS |
| 40250 | DLVNCNNAN |
| 40251 | DMAFMHSIC |
| 40252 | DMCKKMLSW |
| 40253 | DMIGSQAEL |
| 40254 | DMVYLAGNA |
| 40255 | DNEDVLHMG |
| 40256 | DNGWFENMA |
| 40257 | DNQCNEITL |
| 40258 | DNRDQHWRT |
| 40259 | DNRNERRQS |
| 40260 | DNTVNTIIW |
| 40261 | DNVGRVNSV |
| 40262 | DNWKPTRIG |
| 40263 | DPEQAYTKH |
| 40264 | DPLLKSEWG |
| 40265 | DPNNYKGYY |
| 40266 | DPPLHWVTM |
| 40267 | DQAFEWFIY |
| 40268 | DQCKTLFYT |
| 40269 | DQGQVGCFV |
| 40270 | DQGYMPTKT |
| 40271 | DQLNQQAMS |
| 40272 | DQPFMNRQH |
| 40273 | DQRICGEKC |
| 40274 | DQWLMQCKN |
| 40275 | DRCNKIWPD |
| 40276 | DRGWQNWTY |
| 40277 | DRKQAQRNG |
| 40278 | DRNTIRKMA |
| 40279 | DRPHGQMMT |
| 40280 | DSKLLCCSC |
| 40281 | DSKMAGQAN |
| 40282 | DSKQAFVMT |
| 40283 | DSKTIQVYW |
| 40284 | DSLIQDFLH |
| 40285 | DSLLANQDG |
| 40286 | DSQYQAIQF |
| 40287 | DSRHLACYT |
| 40288 | DSRILQAGD |
| 40289 | DSRMCPPYN |
| 40290 | DSRQPNAGL |
| 40291 | DSRVDSDDY |
| 40292 | DSVNSCAFP |
| 40293 | DTDWIDCMG |
| 40294 | DTEFRVMDW |
| 40295 | DTISEQMRT |
| 40296 | DTMLKFVST |
| 40297 | DTNIDLKPP |
| 40298 | DTRIDSVYE |
| 40299 | DTRQWDDGL |
| 40300 | DTSQYGNHV |
| 40301 | DTVQLMGSQ |
| 40302 | DTYELESQL |
| 40303 | DVAQWFEAS |
| 40304 | DVARCMDCQ |
| 40305 | DVASKDDWP |
| 40306 | DVCKYQHNP |
| 40307 | DVCYAGLTE |
| 40308 | DVDDRMEMC |
| 40309 | DVFNAGPPF |
| 40310 | DVHVPCVIG |
| 40311 | DVIMCNCPK |
| 40312 | DVMSMFTDV |
| 40313 | DVRSGNFYP |
| 40314 | DVSICVTCT |
| 40315 | DVSWHYAQG |
| 40316 | DVTDNGTCC |
| 40317 | DVVNEDTGP |
| 40318 | DVWEHSHPS |
| 40319 | DVWTPWNAS |
| 40320 | DWAKYWHFP |
| 40321 | DWESDYYQN |
| 40322 | DWFQCHCAA |
| 40323 | DWILELMST |
| 40324 | DWKLFNAWK |
| 40325 | DWMEGSENG |
| 40326 | DYELTPSWY |
| 40327 | DYGESKLEP |
| 40328 | DYGMPECVD |
| 40329 | DYILLTTWE |
| 40330 | DYRWIKGLA |
| 40331 | DYSQFGGGQ |
| 40332 | EAAFIDAHO |
| 40333 | EADWDSCWM |
| 40334 | EAEWVLCCK |
| 40335 | EAGPLKDCC |
| 40336 | EAHMRRYSK |
| 40337 | EAHSVRYLM |
| 40338 | EALACQDPG |
| 40339 | EAMRRSNLH |
| 40340 | EAMRRYRSP |
| 40341 | EANDYNQNC |
| 40342 | EANRIDAVS |
| 40343 | EANRVLCMN |
| 40344 | EAPLPKWQC |
| 40345 | EASCYWEGR |
| 40346 | EASMGRNGH |
| 40347 | EAYPQKECA |
| 40348 | EAYTVSGYA |
| 40349 | ECANHEKTY |
| 40350 | ECDFRMIWD |
| 40351 | ECEFPDVYA |
| 40352 | ECGEFTCML |
| 40353 | ECKVYSGGK |
| 40354 | ECQRPHPIQ |
| 40355 | ECRMIWNHK |
| 40356 | ECRPWVWPG |
| 40357 | ECSAFHWQP |
| 40358 | ECSCEEIPC |
| 40359 | ECSQMISSE |
| 40360 | ECTMTNASN |
| 40361 | ECWNWESFI |
| 40362 | ECWNYLMSW |
| 40363 | EDCLFPACF |
| 40364 | EDDPVSVAD |
| 40365 | EDFHCFPLS |
| 40366 | EDIAPAHGT |
| 40367 | EDIKQQMVC |
| 40368 | EDKRDAWAR |
| 40369 | EDMTVNWSS |
| 40370 | EDQLCHRTH |
| 40371 | EDQWVQMCA |
| 40372 | EDWHTFYWK |
| 40373 | EECVTGLHA |
| 40374 | EEEPLYVDN |
| 40375 | EFDQIGLFN |
| 40376 | EFLLHHCAP |
| 40377 | EFLPKSDQH |
| 40378 | EFMSHSTGA |
| 40379 | EFNPAMWIV |
| 40380 | EFPGKHWVW |
| 40381 | EFRQMGACG |
| 40382 | EFVVLPEAI |
| 40383 | EFYQQAYQH |
| 40384 | EGEQPSMAI |
| 40385 | EGHGCDPWF |
| 40386 | EGIKKEGMP |
| 40387 | EGLNDDSRA |
| 40388 | EGLSMNEYW |
| 40389 | EGQTSSECF |
| 40390 | EGRLTMKHH |
| 40391 | EGYQHPNEF |
| 40392 | EHDPYMKVF |
| 40393 | EHDWPADRI |
| 40394 | EHEQDCEQI |
| 40395 | EHPIDWRKE |
| 40396 | EHQLAHESA |
| 40397 | EHQLPQVDF |
| 40398 | EHRFRVKLA |
| 40399 | EHRQEQTRL |
| 40400 | EIASGSMYW |
| 40401 | EIATNYYSQ |
| 40402 | EICQINHSG |
| 40403 | EIEMDGAHW |
| 40404 | EIEVFWKMH |
| 40405 | EIGIRLGOD |
| 40406 | EIGWHMNYV |
| 40407 | EIGYRSMMT |
| 40408 | EIHEGQLAQ |
| 40409 | EILKPGAHM |
| 40410 | EIMISIGHT |
| 40411 | EIMTRYGGH |
| 40412 | EISCKKWNQ |
| 40413 | EIVYSVFGL |
| 40414 | EIWASQQVN |
| 40415 | EKEFVHSCD |
| 40416 | EKEPLHCDG |
| 40417 | EKFMLWFWG |
| 40418 | EKGCVGPVS |
| 40419 | EKHTMTWHN |
| 40420 | EKKFRTADA |
| 40421 | EKMCEWLTD |
| 40422 | EKMFTLSDS |
| 40423 | EKYGTQREN |
| 40424 | EKYHVDHHA |
| 40425 | EKYTYNMGT |
| 40426 | ELCKGNCFL |
| 40427 | ELFSFGHRE |
| 40428 | ELTLRMNWG |
| 40429 | EMAIRWDYT |
| 40430 | EMAMNGRTG |
| 40431 | EMCGSSKVY |
| 40432 | EMESWDYCQ |
| 40433 | EMGEHHSTL |
| 40434 | EMHMIKKHY |
| 40435 | EMIKTAWAS |
| 40436 | EMTNAETFT |
| 40437 | ENESCMHIE |

TABLE 85
Sequences of the 581 to 589 Region in
AAV5 VP1 Capsid Polypeptide that Drive
Thyroid Gland Tissue Tropism

| SEQ ID | 581-589 |
|---|---|
| NO | Sequence |
| 42438 | KGVWCKIDV |
| 42439 | QQFPHNRQC |
| 42440 | DLKERMRGF |
| 42441 | ISTKWDYIA |
| 42442 | ISWISHASP |
| 42443 | DKTVQTSAR |
| 42444 | RMYATEHCS |
| 42445 | GLWLNVAPR |
| 42446 | SMHYYDTFE |
| 42447 | FNIHGDLLC |
| 42448 | WMSEQMFKG |
| 42449 | ILNMKACDV |
| 42450 | VFKRVVGFA |
| 42451 | QMQNENRQM |
| 42452 | RTREPFWWW |
| 42453 | MQYRGMSAS |
| 42454 | VSDTMNCGS |
| 42455 | EAEPWDYWA |
| 42456 | IAWADGLEF |
| 42457 | IAWCDGLEF |
| 42458 | IAWSDGLEF |
| 42459 | IAWSDGMEF |
| 42460 | IAWYDGLEF |
| 42461 | IVWSDGLEF |
| 42462 | MAWSDGLEF |
| 42463 | MWIEFNEYG |
| 42464 | MWPEFNEYG |
| 42465 | MWTECNEYG |
| 42466 | MWTEFDEYG |
| 42467 | MWTEFNAYG |
| 42468 | MWTEFNECG |
| 42469 | MWTEFNESG |
| 42470 | MWTEFNEYE |
| 42471 | MWTEFNEYV |
| 42472 | MWTEFNGYG |
| 42473 | MWTEFNKYG |
| 42474 | MWTEFNVYG |
| 42475 | MWTEFSEYG |
| 42476 | MWTEFTEYG |
| 42477 | MWTESNEYG |
| 42478 | MWTEVNEYG |
| 42479 | MWTGFNEYG |
| 42480 | SAWSDGLEF |
| 42481 | VWTEFNEYG |
| 42482 | MWAEFNEYG |
| 42483 | MWTEFNEYW |
| 42484 | MWTEFNENG |
| 42485 | MWTEFNEHG |
| 42486 | LFLQLYRHC |
| 42487 | LWRSLQDGN |
| 42488 | QISVYDTKY |
| 42489 | AMHCQPAFA |
| 42490 | NAIPYFVQA |
| 42491 | GLNICKNQQ |
| 42492 | MMVQWGAMA |
| 42493 | MTSTSSRGE |
| 42494 | DGQNRDQMC |
| 42495 | DKIDTCCFL |
| 42496 | NTQVISRWT |
| 42497 | GTMCREQMI |
| 42498 | KTCLCIFTK |
| 42499 | EASIQASWN |
| 42500 | HACSLLQQY |
| 42501 | RVTIMFTGT |
| 42502 | LWGWPLHKL |
| 42503 | TKQFVHNED |
| 42504 | AQIQPAMFP |
| 42505 | KGNSVTHVQ |
| 42506 | LIHWLKHDL |
| 42507 | ICHTEVMEN |
| 42508 | RVMMGNNQA |
| 42509 | SPQQPSVFH |
| 42510 | LTLRQGYIT |
| 42511 | EFVLGCQGI |
| 42512 | YSSHHSMDY |
| 42513 | SVTYHEWHP |
| 42514 | GCLQMPCHE |
| 42515 | MVENGLRLQ |
| 42516 | SRCTVMDPG |
| 42517 | IVQPMCYQQ |
| 42518 | DVKAFMGGV |
| 42519 | AYAPSKNAM |
| 42520 | NVYMRTCNM |
| 42521 | NAAYQMAAY |
| 42522 | LVQKWMRTC |
| 42523 | EKHFMQVDR |
| 42524 | LEIVMHSLP |
| 42525 | NQREWHGLA |
| 42526 | LADGCCCVN |
| 42527 | AVETGESAM |
| 42528 | RCADNPYFR |
| 42529 | ETHTVSREC |
| 42530 | QAAWIPFAG |
| 42531 | AVEDYWRYF |
| 42532 | QYVQCAAKD |
| 42533 | ACIPQMHSA |
| 42534 | AVTGHVIGA |
| 42535 | WGDARMIGP |
| 42536 | TGMAMSEQS |
| 42537 | VFNLMLQDK |
| 42538 | VTCNWCVMP |
| 42539 | DANHCMWWQ |
| 42540 | QISKPSVQC |
| 42541 | WTTSIAVDT |
| 42542 | QTSHQDGDC |
| 42543 | QAKMILDGT |
| 42544 | IHNRYAQAP |
| 42545 | NWYTTWIDE |
| 42546 | CMEVTCFVV |
| 42547 | AYRVKEVAY |
| 42548 | APVHPTQSM |
| 42549 | QKAIVAQND |
| 42550 | AQGFKPSDG |
| 42551 | NANYWFYYD |
| 42552 | TRETMNEWH |
| 42553 | GVWMPHGWR |
| 42554 | AVHNVKDVH |
| 42555 | ETSTHCCKV |
| 42556 | FQDERTTHH |
| 42557 | KSNLCYHVQ |
| 42558 | DTRRNLCCD |
| 42559 | AEHMVVDHE |
| 42560 | DVYTMHELH |
| 42561 | MKEDNKFAV |
| 42562 | MFFPGFVAH |
| 42563 | RFDQFWDTY |
| 42564 | HFQPVFMQP |
| 42565 | DIEFLDCEN |
| 42566 | QCLQTNPYN |
| 42567 | RTMDCEYTG |
| 42568 | MSGPNDTEF |
| 42569 | IGDEYATHT |
| 42570 | SFSDTHLGW |
| 42571 | YFPSIQCYE |
| 42572 | IRLMTPDEN |
| 42573 | ICHIRTRGN |
| 42574 | MCDNFPSAH |
| 42575 | SSIYKEIQE |
| 42576 | PMMIIPYVD |
| 42577 | PLTIEVNCT |
| 42578 | NVCDNGHEI |
| 42579 | KIAAWNDFL |
| 42580 | HQCNCLLSW |
| 42581 | GAANRIQSW |
| 42582 | DVWWTLTRD |
| 42583 | CSESYRGPD |
| 42584 | TISQIPWVR |
| 42585 | MFMTNVNSF |
| 42586 | GKGQWTAQE |
| 42587 | SGALKNREE |
| 42588 | LHSYQWRLG |
| 42589 | DVKSWAVCD |
| 42590 | LPSHCNLGS |
| 42591 | GQQTIEMSY |
| 42592 | QWFTKGVGE |
| 42593 | QRNEMSASR |
| 42594 | YPAQYTAFS |
| 42595 | DHLSNDAEM |
| 42596 | DQEAQVVTG |
| 42597 | ASMHEETCL |
| 42598 | TTYPHNTHG |
| 42599 | QFAQLWCAK |
| 42600 | MTDNNGPLF |
| 42601 | RYPCCQSPF |
| 42602 | CAWHMKQGN |
| 42603 | TSVIRLFWI |
| 42604 | TTTNQFMFQ |
| 42605 | MMLGYMEQD |
| 42606 | GLFEGSPGA |
| 42607 | AEDTPYTIY |
| 42608 | QHKWHDVGV |
| 42609 | NRIQMTDFQ |
| 42610 | YVDHWTLGD |
| 42611 | ACVERLHHK |
| 42612 | DQRHGNVSP |
| 42613 | ATGTYNLQE |
| 42614 | EAGCKMWPT |
| 42615 | KQKTQNRNA |
| 42616 | ASKQGTHDY |
| 42617 | TYESLQNSW |
| 42618 | MNLQIGSKG |
| 42619 | GRALGKKQG |
| 42620 | EVRIQCKID |
| 42621 | PHAETHWYT |
| 42622 | WERCLELNQ |
| 42623 | AGMYKCQED |
| 42624 | TVNWYSGFG |
| 42625 | ATMNLSWGL |
| 42626 | QSSHCKQMV |
| 42627 | YSDPKNMSI |
| 42628 | GHLFNNNEW |
| 42629 | DINTCRVCY |
| 42630 | MSDQKEHMS |
| 42631 | AGCSISWTP |
| 42632 | AVIVTADMQ |
| 42633 | AWQQNTFWY |
| 42634 | AWTQASRGN |
| 42635 | AWYKAWSHV |
| 42636 | DFFLMKIAD |
| 42637 | DLDFWNGQS |
| 42638 | DVQEAMNLV |
| 42639 | DVRLATAWH |
| 42640 | DVYATPVVC |
| 42641 | ECVEGQSCW |
| 42642 | ETKYWWVNF |
| 42643 | EVTQCDFFV |
| 42644 | FTGASFHIA |
| 42645 | GCMYIRHAY |
| 42646 | GIDAHAIIY |
| 42647 | GTMCRERMI |
| 42648 | GYMCIRHAY |
| 42649 | HAYSLLQQY |
| 42650 | HLAPYCQQA |
| 42651 | HLTMWLVMT |
| 42652 | IHDLVPAYD |
| 42653 | IKQFWTKKM |
| 42654 | ITHRYGWNL |
| 42655 | KGMHSMPGI |
| 42656 | KLTIDTACR |
| 42657 | KQAGMLLGW |
| 42658 | KSNDSDNEF |
| 42659 | LMWTRNTAH |
| 42660 | LNIQCTNQA |
| 42661 | LRHANRRTY |
| 42662 | LVKAHCAWS |
| 42663 | MAELCMEGH |
| 42664 | MYMPATMAV |
| 42665 | NAEDVDQGE |
| 42666 | NCCFVYWWL |
| 42667 | NDRHVPCRD |
| 42668 | NLMTDWWDI |
| 42669 | PPEQFQSGK |
| 42670 | PVQKWMRTC |
| 42671 | QACLQIMWN |
| 42672 | QGYRSSEMQ |
| 42673 | QKALNVHHA |
| 42674 | QMHMVHKCE |
| 42675 | QNMLMQAQV |
| 42676 | QYIDTYHWT |
| 42677 | QYQTHPMMH |
| 42678 | RNKSPDECF |
| 42679 | RTAHRDDFA |
| 42680 | RVEYPPFTS |
| 42681 | SFLVMNRMG |
| 42682 | TDLWYEKSS |
| 42683 | TEAKRTIDW |
| 42684 | TGCYHVHMD |
| 42685 | TICKCKISN |
| 42686 | TIQQCMHLG |
| 42687 | TPQNNAMSM |
| 42688 | TRRTCPIAM |
| 42689 | VRWGYTCFY |
| 42690 | YFEHMMITT |
| 42691 | YFHMIDQIP |
| 42692 | YNDFNRGEW |
| 42693 | YSNCQPTHM |
| 42694 | ADGWWMDSL |
| 42695 | AHLEMMNQF |
| 42696 | ALLMFSLSN |
| 42697 | AMLDSHSAF |
| 42698 | AQSRMHTMD |
| 42699 | ARTMYVESF |
| 42700 | ASEYFPVFK |
| 42701 | ASIWPEGKD |
| 42702 | AVICKRFVG |
| 42703 | AVIMTADMQ |
| 42704 | AVKANDTVH |
| 42705 | AWDANLAFV |
| 42706 | CAHTDKVHH |
| 42707 | CEIVVSHVE |
| 42708 | CLRSDIGKQ |
| 42709 | CNEKGYHWH |
| 42710 | CTLSFMEDK |
| 42711 | CVRFTPTHN |
| 42712 | DCAWQTNTS |
| 42713 | DCMCNFAGM |
| 42714 | DFFPMKIAD |
| 42715 | DHRYMMSSV |
| 42716 | DISLKLCHQ |
| 42717 | DITWPNHSY |
| 42718 | EEGATSGWP |
| 42719 | EIQPMMRKP |
| 42720 | EMTLQSVSS |
| 42721 | EPKYWWVNF |
| 42722 | ESILPGKIL |
| 42723 | FASLCNNGL |
| 42724 | FASLSGPPN |
| 42725 | FTCNHLAFY |
| 42726 | GEEADEYHM |
| 42727 | GGMAHAIIN |
| 42728 | GLENVVVGL |
| 42729 | GNDFFVNIY |
| 42730 | HASSEHVQD |
| 42731 | HMGPYMESE |
| 42732 | IAMRTLNQK |
| 42733 | IAMTAILCD |
| 42734 | IATWGEQQM |
| 42735 | IECEWPQDE |
| 42736 | IECKWPQDK |
| 42737 | ITPKFMFPS |
| 42738 | KASEHLPGF |
| 42739 | KGLTIQKYA |
| 42740 | KMCTHDLAC |
| 42741 | KPQPTGIFT |
| 42742 | KTDFVFLPQ |
| 42743 | KVVPAEHQY |
| 42744 | LAGPKLWEQ |
| 42745 | LAHMIEITA |
| 42746 | LCGHCGESC |
| 42747 | LCKYCFTAD |
| 42748 | LRSLWEAWH |
| 42749 | LTKFLHKGD |
| 42750 | MGYKRAASR |
| 42751 | MMFRPNSAH |
| 42752 | MNKFREEYG |
| 42753 | MPQGAVNSD |
| 42754 | MSSFMASRE |
| 42755 | MTNNKMMYE |
| 42756 | NCVVNLCTY |
| 42757 | NEPLFMMGS |
| 42758 | NGVWCKIDV |
| 42759 | NLSPINWHL |
| 42760 | NOLPEMIET |
| 42761 | NRDGVPQSY |
| 42762 | NSSMIEWDC |
| 42763 | NTAHLQDGW |
| 42764 | NTRLVFGIT |
| 42765 | NTSIWERDQ |
| 42766 | NTWVPHEFY |
| 42767 | NVFVAYCKS |
| 42768 | NVMRVPTGQ |
| 42769 | PRNQLGPGI |
| 42770 | PSSQFKCDQ |
| 42771 | PTIACGNMV |
| 42772 | PWKYCCMDQ |
| 42773 | PYVDKWANH |
| 42774 | QIHRAGAKQ |
| 42775 | QNRLVNRQH |
| 42776 | QRDFAWQFC |
| 42777 | QRQGQPHYW |
| 42778 | QSFSYDAKE |
| 42779 | RFKTCRYMY |
| 42780 | RKVRIDGDT |
| 42781 | RNEKGYHWH |
| 42782 | RPEDVPVAP |
| 42783 | RSFMCGLIG |
| 42784 | RYKSRGALT |
| 42785 | SATILANEV |
| 42786 | SIVDCQGAF |
| 42787 | SLVPVSKTR |
| 42788 | SWTCCATQC |
| 42789 | SYDEICSTV |
| 42790 | TAILNQEIH |
| 42791 | TFSPARHPK |
| 42792 | TGHPLSNCL |
| 42793 | TLGYVWSVG |
| 42794 | TNERFTIQC |
| 42795 | TQDIWYNYV |
| 42796 | VCYCSSTAK |
| 42797 | VDQPTTHWG |
| 42798 | VHGQYPCQE |
| 42799 | VHNWFLTWW |
| 42800 | VLTTIWKNN |
| 42801 | VNAQQNWGV |
| 42802 | WANTQWQDD |
| 42803 | WGYNYQCAF |
| 42804 | WLKYKKTWI |
| 42805 | WPDQNMFAV |
| 42806 | WYTFLQFIT |
| 42807 | YCNAYMIGM |
| 42808 | MEHVVSVQP |
| 42809 | NTRFPDQTE |
| 42810 | ACLSPDHDR |
| 42811 | AMCMLSHIH |
| 42812 | ANRNCVTPH |
| 42813 | ARMINDNDP |
| 42814 | ATWSADVTA |
| 42815 | CNFTVCNMS |
| 42816 | CTAEMAPMT |
| 42817 | CTDGKHASW |
| 42818 | DEISDPEEF |
| 42819 | DGAQDKWAC |
| 42820 | DLIYKGQNG |
| 42821 | DRPKRQVLY |
| 42822 | DSHIDQFGF |
| 42823 | DTEKGQAWW |
| 42824 | EDILEITKH |
| 42825 | EMAGMREFQ |
| 42826 | ENNPSHTQL |
| 42827 | EPYKQMGAG |
| 42828 | ERTLTKAGS |
| 42829 | ETVMMEVRD |
| 42830 | EVTKCEPPS |
| 42831 | EVTKCGPPS |
| 42832 | GQKSYDGCH |
| 42833 | GVDNQDFSW |
| 42834 | HAKCNRNIK |
| 42835 | HQHDDRPDM |
| 42836 | HVMEWLQCP |
| 42837 | IAGPIEIGM |
| 42838 | IKQYPQNKP |
| 42839 | IQAVMASGW |
| 42840 | ITDEYFMNM |
| 42841 | KACSMQQEW |
| 42842 | KGVALCDYQ |
| 42843 | KIYMQCDQE |
| 42844 | KPEWNHTSA |
| 42845 | LANHAKMGL |
| 42846 | LFSKEKHHR |
| 42847 | LMSPYNFLT |
| 42848 | LMYQDDMFE |
| 42849 | LQHQLHVYE |
| 42850 | LSQCQSYVA |
| 42851 | LTKPCWPWA |
| 42852 | MAAGNTTCA |
| 42853 | MCSSDLEGC |
| 42854 | MDCCARQAP |
| 42855 | MPSCINGPY |
| 42856 | MTNRCAKYK |
| 42857 | MVTFRENPA |
| 42858 | NASVGAWAQ |
| 42859 | NEQVATPMA |
| 42860 | NIGYMNVLF |
| 42861 | NLRECTIMN |
| 42862 | NSLLRHGEQ |
| 42863 | QLHQCPAVG |
| 42864 | QSQPDHVSW |
| 42865 | QYECESDNS |
| 42866 | RNDYSDQYD |
| 42867 | RSDQHSPHM |
| 42868 | SCORNQQTN |
| 42869 | SFHNCMLDP |
| 42870 | SSTNNQTVD |
| 42871 | STCVTEMFY |
| 42872 | TCFNQNNTW |
| 42873 | TFFQAVVIA |
| 42874 | TMASWECYQ |
| 42875 | TMDSIQWFE |
| 42876 | TQKAHKNPG |
| 42877 | TTDSKLLGH |
| 42878 | TVEDRRKQG |
| 42879 | VADCPHHFD |
| 42880 | VHPYAESVD |
| 42881 | VVMVQHRTA |
| 42882 | VVPHPFPAY |
| 42883 | WWEACPAAS |
| 42884 | YCTTLSMES |
| 42885 | YFVMQWFHY |
| 42886 | YMVQDRDEW |
| 42887 | YMYSNMQTM |
| 42888 | GTSEWMHMA |
| 42889 | TVGTAKSQI |
| 42890 | DEKFGQVED |
| 42891 | GTPDMRLTL |
| 42892 | STAFASSWE |
| 42893 | HIQCQGDSW |
| 42894 | NNDIRIFGN |
| 42895 | WAPLPYNWC |
| 42896 | LVDGYRMRW |
| 42897 | DLCVHANVR |
| 42898 | TLLPHWQAT |
| 42899 | SIPMGNNWG |
| 42900 | FNTLGLLFD |
| 42901 | ENMHKMGML |
| 42902 | RNSHGWRLD |
| 42903 | QCARPWYRS |
| 42904 | MWGWPGDAF |
| 42905 | KVIRKDSCV |
| 42906 | MSPNTKDCT |
| 42907 | NGRLWWQRY |
| 42908 | TYSQPPVSG |
| 42909 | HFTHTESAH |
| 42910 | SRFWNRWDF |
| 42911 | CLGPWNVKS |
| 42912 | CCMSKMPDE |
| 42913 | MTHPYWEDS |
| 42914 | THNVKTDYP |
| 42915 | LDYHFPFEA |
| 42916 | WETLEHWRG |
| 42917 | DANWRTWQH |
| 42918 | CVVPYYRFT |
| 42919 | HHEPYGSNC |
| 42920 | THHHVLEVP |
| 42921 | AAMILESHK |
| 42922 | CMRGKWDRL |
| 42923 | VTMIYDLHQ |
| 42924 | LDHHAEVLQ |
| 42925 | MFYPFQCGN |
| 42926 | FASHQMNMM |
| 42927 | PPESQESST |
| 42928 | PDPKAWSNY |
| 42929 | IRDLVPAYD |
| 42930 | QCPCSRLIC |
| 42931 | TICKCTISN |
| 42932 | DEEFCEMSA |
| 42933 | LPRPGILME |
| 42934 | PYGKAAEKL |
| 42935 | EKMRYPPMI |
| 42936 | SFRCCWDWH |
| 42937 | KPPGSRWIT |
| 42938 | LACVAHWPS |
| 42939 | MSHEMFWVD |
| 42940 | KNTMCDNDA |
| 42941 | AVKMPVEQS |
| 42942 | FTANEDQCH |
| 42943 | CRRGQLLYE |
| 42944 | DKIMMAEMD |
| 42945 | PIAFGCERA |
| 42946 | ETGYRENPC |
| 42947 | RFADYCKEE |
| 42948 | NWVFYTNFP |
| 42949 | WECQIQDMK |
| 42950 | DIYHFNHRS |
| 42951 | NSFANNFVM |
| 42952 | DAQWSFEGA |
| 42953 | RINCNYGQM |
| 42954 | CHPPMTNMG |
| 42955 | WPDSNMTGV |
| 42956 | QTIMRHAQT |
| 42957 | VERLCACQA |
| 42958 | VMMYNDSIS |
| 42959 | AWDANLAFA |
| 42960 | DAGKFGVDI |
| 42961 | NQSVCYSSV |
| 42962 | LRHDVFSQA |
| 42963 | DFAAQQRAQ |
| 42964 | SAGPFFDHY |
| 42965 | CMASVNYFD |
| 42966 | CWARMQGYM |
| 42967 | CFVDSVFMY |
| 42968 | APRNVHCGT |
| 42969 | GRCFMPPGR |
| 42970 | KNHAHEFIE |
| 42971 | IIAVSMRGR |
| 42972 | LPRMPECSV |
| 42973 | TTMVQMGAW |
| 42974 | TLVSCDQGN |
| 42975 | SADRMISHE |
| 42976 | MPHHSPDCW |
| 42977 | TPSQMDMDT |
| 42978 | SCNFEFLWM |
| 42979 | TTVDYAGTG |
| 42980 | WKCCQDQLL |
| 42981 | SWFAGQWEW |
| 42982 | EITDKYWGE |
| 42983 | CRGSLSNAN |
| 42984 | KAYMMMEGP |
| 42985 | VPLGFNFAG |
| 42986 | AVSNWLETS |
| 42987 | MWTVARMFP |
| 42988 | ITHKYGWNL |
| 42989 | QGRWACWYS |
| 42990 | NHASFKRFM |
| 42991 | RNMALHHPT |
| 42992 | MWTKFNEYG |
| 42993 | HLLKKDASD |
| 42994 | GQAGVHNLS |
| 42995 | SFFREDYSF |
| 42996 | RCTDCSYFY |
| 42997 | IKRAYDNNG |
| 42998 | SQQHCQHDQ |
| 42999 | QGDFFNICP |
| 43000 | CMQKYIPDV |
| 43001 | PSLWKEKVW |
| 43002 | YGTAMDSLY |
| 43003 | IHQCIVCAP |
| 43004 | HLAPYCQQE |
| 43005 | LMQSGQKDF |
| 43006 | EVDQYYSLA |
| 43007 | KIQMAVSLD |
| 43008 | LINQWMHDN |
| 43009 | HTHDVRVHK |
| 43010 | MPASNWVAE |
| 43011 | RTVHRDDFA |
| 43012 | TSRDGGWAY |
| 43013 | KNETFIAST |
| 43014 | EVAGCATAV |
| 43015 | MWTEFNEDG |
| 43016 | QPGHTYNAP |
| 43017 | MESHHHAHA |
| 43018 | DCVCCSELV |
| 43019 | TKQPVHFLG |
| 43020 | AMNWWDAPQ |
| 43021 | YFMTCSTYP |
| 43022 | GSFMSCEKN |
| 43023 | VDQHGWNDM |
| 43024 | HHSWQPQKY |
| 43025 | QFCTSCASN |
| 43026 | KMLLVMDGD |
| 43027 | ERTCIATMW |
| 43028 | GVDNRQWLF |
| 43029 | NEAPYSTWQ |
| 43030 | SGNIDVHTM |
| 43031 | AADNYSWVV |
| 43032 | AAHRGECMA |
| 43033 | AAVPRMWAM |
| 43034 | AAWGYNQFF |
| 43035 | AAWVDKWWV |
| 43036 | AAYMSSECL |
| 43037 | ACCCHENVL |
| 43038 | ACEHPLNYD |
| 43039 | ACGVTQMNP |
| 43040 | ACQYDVETL |
| 43041 | AEDVDEGCD |
| 43042 | AEIRNVACW |
| 43043 | AFFMDHMWH |
| 43044 | AGINCQWCP |
| 43045 | AHFPFFVYW |
| 43046 | AIFSREENG |
| 43047 | AIGIDHQAF |
| 43048 | AIGNFHMAY |
| 43049 | AIIWGAIQE |
| 43050 | AIQMPDVTY |
| 43051 | AKDMHVANE |
| 43052 | AKLSNCAIP |
| 43053 | AKMGFDRFT |
| 43054 | AKSHHDDSA |
| 43055 | ALEYIRQGT |
| 43056 | ALHHYPVYH |
| 43057 | ALHVVMDVG |
| 43058 | AMDYLMEHV |
| 43059 | AMGNQRNYY |
| 43060 | AMHAQQNRE |
| 43061 | AMIASHHCQ |
| 43062 | AMKFAGECA |
| 43063 | AMKFAGEGA |
| 43064 | AMMHRNNET |
| 43065 | ANAYQINGQ |
| 43066 | ANCMDFHIT |
| 43067 | ANFYNEEAT |
| 43068 | ANHHQTYDT |
| 43069 | ANQISVRGV |
| 43070 | APCWEGSMR |
| 43071 | APESARPAN |
| 43072 | APIWNDSLM |
| 43073 | APQVSKRFA |
| 43074 | APSTFANWG |
| 43075 | APVNMENPI |
| 43076 | AQQCSRLMF |
| 43077 | AQRPCDDWQ |
| 43078 | AQTMSHCFW |
| 43079 | ARAAQLSYW |
| 43080 | ARQCNPNCT |
| 43081 | ASLDQAEQN |
| 43082 | ATENEHAMP |
| 43083 | ATNQRKAYT |
| 43084 | ATTNMFVDT |
| 43085 | ATVKYNCHW |
| 43086 | AVAFGSQED |
| 43087 | AVGFCQYSS |
| 43088 | AVNHTPIWA |
| 43089 | AVNYGHEGG |
| 43090 | AVTDKCDNP |
| 43091 | AVTSYDNMQ |
| 43092 | AWTLNNNHW |
| 43093 | AYTDIWNFW |
| 43094 | CAAMAYEKG |
| 43095 | CAEVQGETS |
| 43096 | CASGFGRLA |
| 43097 | CAWVMQWHH |
| 43098 | CCHFDEFYM |
| 43099 | CDNSENFLT |
| 43100 | CDTRWYCLC |
| 43101 | CEAEKVEQP |
| 43102 | CEGVANPWE |
| 43103 | CFPLCSFST |
| 43104 | CFTPHESMQ |
| 43105 | CGESWAGEN |
| 43106 | CGPNVSMMP |
| 43107 | CGVVESGMN |
| 43108 | CHKPYHLGI |
| 43109 | CHYEYSKFG |
| 43110 | CIETVHKHF |
| 43111 | CIWPYQAIQ |
| 43112 | CKPLECQLV |
| 43113 | CLCLIVDCE |
| 43114 | CLLGEVMFD |
| 43115 | CLTDSQDLP |
| 43116 | CMHWGWIPM |
| 43117 | CMKIFKFNS |
| 43118 | CMMTYMHLG |
| 43119 | CMNCRHQCL |
| 43120 | CMQKIDHSW |
| 43121 | CMSVSTRWY |
| 43122 | CMVCPGREE |
| 43123 | CNDRYMFAH |
| 43124 | CPCTFADEQ |
| 43125 | CPKPSDGSF |
| 43126 | CPLTSWHFE |
| 43127 | CQHRTHCCM |
| 43128 | CQIPEGHEG |
| 43129 | CQVKRNATG |
| 43130 | CSLRLIAES |
| 43131 | CTALCYSRQ |
| 43132 | CTVIDINKN |
| 43133 | CTWLTQHQF |
| 43134 | CVAIENVNF |
| 43135 | CVRQTVTLC |
| 43136 | CVSHYASQM |
| 43137 | CWPFAINHI |
| 43138 | CWTMFCPPT |
| 43139 | CWTQMKFWG |
| 43140 | CYHQYETRC |
| 43141 | CYKSTQGAE |
| 43142 | DAGDENAPE |
| 43143 | DAKMGAEFS |
| 43144 | DAKNECASW |
| 43145 | DALLHRHYE |
| 43146 | DAREYWRYQ |
| 43147 | DASPKGGAS |
| 43148 | DAWLHEQGT |
| 43149 | DCNAADPGR |
| 43150 | DCQYVVNDC |
| 43151 | DCRMCERHV |
| 43152 | DCRWMSIMG |
| 43153 | DDGKTYCLY |
| 43154 | DDRNVWSAD |
| 43155 | DDVRQGDCW |
| 43156 | DEIDIGREG |
| 43157 | DEMLKQFNG |
| 43158 | DFNVTAFRD |
| 43159 | DFVGCSHWG |
| 43160 | DGTPISCVG |
| 43161 | DHDDTRLHR |
| 43162 | DHDKREIVT |
| 43163 | DHFYIAKQY |
| 43164 | DHGFEGGWP |
| 43165 | DIWHTQDME |
| 43166 | DKAQIKMYG |
| 43167 | DKDEHDTQD |
| 43168 | DKKQSCDTS |
| 43169 | DKQLMMLHT |
| 43170 | DKRWQKMVR |
| 43171 | DKSQMHKWG |
| 43172 | DLCFMEQLH |
| 43173 | DLEVTSASQ |
| 43174 | DLHDVVFVV |
| 43175 | DMHGIENAR |
| 43176 | DMPEDSIHW |
| 43177 | DNKSIVMSQ |
| 43178 | DNNVQKCGQ |
| 43179 | DNQPTVCHT |
| 43180 | DNRPPLFWQ |
| 43181 | DPEYTVVLT |
| 43182 | DQHPSFLGC |
| 43183 | DQNESGFGC |
| 43184 | DQWQATIAE |
| 43185 | DRCQTVIDW |
| 43186 | DRKCMIPNK |
| 43187 | DRQQMDCYQ |
| 43188 | DRTPLHQQQ |
| 43189 | DSAELHWPH |
| 43190 | DSFTFLKKA |
| 43191 | DSGILETCD |
| 43192 | DSHAYWMYP |
| 43193 | DSNFSYLGH |
| 43194 | DSQFKMAAW |
| 43195 | DTAAQKWEA |
| 43196 | DTDTVLQSS |
| 43197 | DTEWNHHNT |
| 43198 | DTFQPHWYS |
| 43199 | DTKHSSYDS |
| 43200 | DTMTKEKRH |
| 43201 | DTMVDEYDE |
| 43202 | DVCSTFGWM |
| 43203 | DVDQEQDPI |
| 43204 | DVGIHEWCT |
| 43205 | DVGLSGFER |
| 43206 | DVHLFGEFF |
| 43207 | DVLFEGQGS |
| 43208 | DVNPASHFC |
| 43209 | DVQSWCRQG |
| 43210 | DWKPEMDGS |
| 43211 | DWRMKKSSY |
| 43212 | DWSHTQYGA |
| 43213 | DWVCMDWCQ |
| 43214 | EADDHDQNR |
| 43215 | EAHISAEFK |
| 43216 | EAIFHDVEM |
| 43217 | EAKTSYRMW |
| 43218 | EARETAIMC |
| 43219 | EATQHETRV |
| 43220 | ECMCVQFCQ |
| 43221 | ECTMIEYMG |
| 43222 | EDCNTNERS |
| 43223 | EDIQFDGDS |
| 43224 | EDKTQEMWI |
| 43225 | EDLLMNSNT |
| 43226 | EEPAMKIFM |
| 43227 | EFEHFNYQD |
| 43228 | EFRWQQSTP |
| 43229 | EFTDQADWE |
| 43230 | EFTQDNWGQ |
| 43231 | EFYPSTPCS |
| 43232 | EGNWWHEYA |
| 43233 | EGTDGDMFS |
| 43234 | EHESMCCGW |
| 43235 | EHKLWEGFA |
| 43236 | EHYQKHCYL |
| 43237 | EIFQAFLLS |
| 43238 | EIQRNHAIQ |
| 43239 | EIWMMNGEY |
| 43240 | EKAELVRDL |
| 43241 | EKCLLMNAR |
| 43242 | EKLYSTLQP |
| 43243 | ELAEYGNGW |
| 43244 | ELEVKSLWW |
| 43245 | ELEWMMHCA |
| 43246 | EMMYKKLDQ |
| 43247 | EMTMEGQDI |
| 43248 | EMVQGDTHE |
| 43249 | ENDFLCAWT |
| 43250 | ENMWGYANH |
| 43251 | ENNEYWHVE |
| 43252 | EPEMQNQNQ |
| 43253 | EPRVHTWRK |
| 43254 | ERDYCAYTS |
| 43255 | ERKMCFCHP |
| 43256 | ESAWKHFVP |
| 43257 | ESKWNNPDA |
| 43258 | ESMLAIMST |
| 43259 | ESNRMMGCG |
| 43260 | ESRANATLR |
| 43261 | ESVHEGTSS |
| 43262 | ESYPNNHDL |
| 43263 | ESYYKPDFQ |
| 43264 | ETFFLPNCN |
| 43265 | ETMCGNELG |
| 43266 | ETMDREEDK |
| 43267 | ETMFYHIDH |
| 43268 | ETQAGAPCN |
| 43269 | ETQSAAYDE |
| 43270 | ETTWQCIDP |
| 43271 | EVAQNTPHQ |
| 43272 | EVGDCMPQE |
| 43273 | EVGQHEATI |
| 43274 | EVSTQTKLS |
| 43275 | EVTYWQEWT |
| 43276 | EVYQGMLYF |
| 43277 | EVYQQQKDH |
| 43278 | EWKMWYLQG |
| 43279 | EWTDWRDEG |
| 43280 | EYEFEGNAP |
| 43281 | EYRQAQSFS |
| 43282 | FAEQEHMAS |
| 43283 | FAIQEYHIT |
| 43284 | FATLQAAQD |
| 43285 | FATTTYADC |
| 43286 | FCHYLVYDD |
| 43287 | FFRCEKITA |
| 43288 | FISTFERHI |
| 43289 | FITEIQDYT |
| 43290 | FITRHPVYD |
| 43291 | FKYMWNFSN |
| 43292 | FLKNKDCLL |
| 43293 | FLLQYMQPS |
| 43294 | FLVQLSDGV |
| 43295 | FMGFCGPYQ |
| 43296 | FMNPIGVNG |
| 43297 | FMSSRVINF |
| 43298 | FPAFPKEAC |
| 43299 | FQHDRHTYL |
| 43300 | FQIPFCRSF |
| 43301 | FQMHHKWDL |
| 43302 | FSAMYPHMN |
| 43303 | FTALLHYDI |
| 43304 | FTAYMQEFA |
| 43305 | FTCPVSHFE |
| 43306 | FTPWPQCCQ |
| 43307 | FTTQFIDGK |
| 43308 | FTWTQCVVP |
| 43309 | FVEVRMMCH |
| 43310 | FVKMRLSDD |
| 43311 | FVTVWRTTG |
| 43312 | FVWNTMSTE |
| 43313 | FYLPMEHMD |
| 43314 | FYTETNHAP |
| 43315 | GAGLYMVMG |
| 43316 | GATCNMFSY |
| 43317 | GAVWQQGSD |
| 43318 | GDSQLNHWM |
| 43319 | GEVEFVPRN |
| 43320 | GFTTMYKHG |
| 43321 | GHEVQNWPM |
| 43322 | GHHQRTTIN |
| 43323 | GHKHGIFAG |
| 43324 | GHKTPSGPG |
| 43325 | GHMCDCTSM |
| 43326 | GIDENHAGL |
| 43327 | GIGCPQQWE |
| 43328 | GIIPWMHFM |
| 43329 | GIKANIGTK |
| 43330 | GKCENKLST |
| 43331 | GKHKTYEPS |
| 43332 | GLQAFCFQS |
| 43333 | GLQHYGEID |
| 43334 | GMCENVSSF |
| 43335 | GMPEDSIHW |
| 43336 | GMQYIHVTG |
| 43337 | GPPMYKDCM |
| 43338 | GPYKNITFQ |
| 43339 | GQGFRRQHR |
| 43340 | GQVEMFADR |
| 43341 | GQVSSTWWH |
| 43342 | GRNNNTTEQ |
| 43343 | GSAPFQCIG |
| 43344 | GSHQFSGPH |
| 43345 | GSNPPMMQY |
| 43346 | GTGTCMWAE |
| 43347 | GTQPVNWLI |
| 43348 | GTVMRGMAL |
| 43349 | GVAPLMDQA |
| 43350 | GVEFQHNQA |
| 43351 | GVGCEILGA |
| 43352 | GVHPDNWRN |
| 43353 | GVQLAMSDC |
| 43354 | GVQSLHFNS |
| 43355 | GVTCKDMYH |
| 43356 | GWSQEGHFA |
| 43357 | GWWGLVHNQ |
| 43358 | GWWYYGQCR |
| 43359 | GYASHQHWQ |
| 43360 | HAGCSTRTG |
| 43361 | HAIHKWPRW |
| 43362 | HALGRVANR |
| 43363 | HAMLNEICD |
| 43364 | HATQRSADA |
| 43365 | HAYLAGWCN |
| 43366 | HCQDLQIGM |
| 43367 | HCWAQYLRF |
| 43368 | HGARGEQFA |
| 43369 | HGCYVAHCG |
| 43370 | HGEMCAFFQ |
| 43371 | HGRCIWPMW |
| 43372 | HGYADCKAH |
| 43373 | HHCNTSEFV |
| 43374 | HHEIKVIFE |
| 43375 | HHILNVWTE |
| 43376 | HHNFRMAMN |
| 43377 | HIAIVCLMD |
| 43378 | HLLHVCLEK |
| 43379 | HMIRGYCDY |
| 43380 | HNGMDTQWH |
| 43381 | HNHHECRQM |
| 43382 | HPPPLPWQS |
| 43383 | HQDQPEWAK |
| 43384 | HRIAEICRP |
| 43385 | HSEMLDLQE |
| 43386 | HSQGFDVTQ |
| 43387 | HTADMRAYD |
| 43388 | HTLFHRDCA |
| 43389 | HVATKEECT |
| 43390 | HVEPLEETM |
| 43391 | HYDPYNRTL |
| 43392 | HYYTPDRMT |
| 43393 | IACMENLSL |
| 43394 | IAFWIDGQC |
| 43395 | IAHWCHKCW |
| 43396 | IATGRESAI |
| 43397 | IATGWESAI |
| 43398 | IAWEKCDAW |
| 43399 | ICHSGGWST |
| 43400 | ICRREWNPV |
| 43401 | IDWVGHMGA |
| 43402 | IECNYCKFY |
| 43403 | IECYKGMFC |
| 43404 | IEPEGHLVT |
| 43405 | IEYPNKWRW |
| 43406 | IFAVMADMS |
| 43407 | IFHPQWKQG |
| 43408 | IFKHCKHDE |
| 43409 | IGAGFYYKP |
| 43410 | IGEAFASTG |
| 43411 | IGLLTSLHD |
| 43412 | IGRVKCART |
| 43413 | IGVMPGIVA |
| 43414 | IHSAHHWAS |
| 43415 | IHSQMIEAP |
| 43416 | IIGRFESMS |
| 43417 | IINPLRHGD |
| 43418 | ILDPQHWSM |
| 43419 | ILTHVSDAC |
| 43420 | IMGNYIQAN |
| 43421 | IMMDWEGNW |
| 43422 | IMWVLNELE |
| 43423 | INEGIGRHF |
| 43424 | INHGTTATF |
| 43425 | INMPAVMKF |
| 43426 | IPGTRIAWA |
| 43427 | IQCYYKGEQ |
| 43428 | IRFGSREAK |
| 43429 | IRGQANSCV |
| 43430 | ISEVENWYD |
| 43431 | ISNKLTMPV |
| 43432 | ISTMIEYSN |
| 43433 | ITEYAQQDC |
| 43435 | ITKIMRQAE |
| 43436 | IVMPVAAFH |
| 43437 | IVQCFVDSQ |

TABLE 86
Sequences of the 581 to 589 Region in
AAV5 VP1 Capsid Polypeptide that Drive
Liver Tissue Tropism

| SEQ ID | 581-589 |
|---|---|
| NO | Sequence |
| 45438 | AFEKVNCPD |
| 45439 | ILIFIQNCP |
| 45440 | ASQRTHHVD |
| 45441 | ATNLINKLF |
| 45442 | EVLLGCQNY |
| 45443 | FYMLSNQGS |
| 45444 | GSVGMLDGQ |
| 45445 | GVNIMFRQQ |
| 45446 | KGEPGMNYY |
| 45447 | KIAVYADWA |
| 45448 | KNAWMDCVN |
| 45449 | KNGAGDAHY |
| 45450 | LAKMYWDMG |
| 45451 | LTQSFQTYH |
| 45452 | MIVQQSTGC |
| 45453 | NRDYYRGNY |
| 45454 | QAVQLWSHV |
| 45455 | QCGSMQGVY |
| 45456 | REAEYWRDQ |
| 45457 | SSFYAQREG |
| 45458 | VIISRHMDS |
| 45459 | VTHLFGSQA |
| 45460 | AAEAGIHQA |
| 45461 | ACQSYWNEA |
| 45462 | DATFCTYMR |
| 45463 | DKTSRLADQ |
| 45464 | KANMCEKIC |
| 45465 | KPNNFDTQF |
| 45466 | NINKFTCHE |
| 45467 | NSAIEYQNE |
| 45468 | QATPSEWEN |
| 45469 | QSLMCWNMG |
| 45470 | SARYQIEGC |
| 45471 | SIVHSGLSF |
| 45472 | TFYPTQNQH |
| 45473 | TSNWLNFFP |
| 45474 | VAAQSDTVN |
| 45475 | VFKLISNQA |
| 45476 | VVVRSNECA |
| 45477 | YHNQDRHYL |
| 45478 | HIKVEHEEV |
| 45479 | ACASTSPNA |
| 45480 | AHSRMDWTP |
| 45481 | AIGRRRLTH |
| 45482 | AKGHDKACC |
| 45483 | AMGSLMRAS |
| 45484 | ARNSMPFGM |
| 45485 | CGTGEDCED |
| 45486 | DKCNVTRAD |
| 45487 | DMSDCYEHA |
| 45488 | DNILRDVMA |
| 45489 | DVANHITQM |
| 45490 | DVDWNMGVE |
| 45491 | DVNQYTMRH |
| 45492 | DWRNKRNHA |
| 45493 | DYVFWGFPC |
| 45494 | EAAKLVEPT |
| 45495 | ECHVCTHKA |
| 45496 | ELPRYYGQM |
| 45497 | ETHRRCKMS |
| 45498 | FAKHCVMTA |
| 45499 | FDEALWKNN |
| 45500 | FTMRMASGF |
| 45501 | GTHTQQWHH |
| 45502 | HVFFAPLEG |
| 45503 | HYMGLLCGC |
| 45504 | IFDVYARWT |
| 45505 | IHIGKNDNC |
| 45506 | IHKFCFKGY |
| 45507 | ISGFTKHEW |
| 45508 | IVKQCYDCH |
| 45509 | KAANDHHVA |
| 45510 | KHVILRQLP |
| 45511 | KIVSNESMT |
| 45512 | KVDGCQKLC |
| 45513 | LMYGVGTCF |
| 45514 | LNNHHGTGS |
| 45515 | MFQWRWIDG |
| 45516 | MGFPKMSAW |
| 45517 | MQVVLQHTS |
| 45518 | MYYQIENDP |
| 45519 | NSICMGGWK |
| 45520 | NVCTKRENE |
| 45521 | NYCKYNKMR |
| 45522 | PESVKMDCN |
| 45523 | QVYYVHTER |
| 45524 | RASHDGCCD |
| 45525 | RIFWDFHAT |
| 45526 | RVPRCVYEC |
| 45527 | TDALTDSMH |
| 45528 | TEDDRWRHA |
| 45529 | TIWERQSFH |
| 45530 | TQGMYVESY |
| 45531 | TSNIFQRHG |
| 45532 | TVNVDSQYN |
| 45533 | VASSHRESQ |
| 45534 | VIKVNTVNP |
| 45535 | VSGMLAACN |
| 45536 | VVNVWGCAP |
| 45537 | WNIVLMFYC |
| 45538 | WWVVVPCFM |
| 45539 | YCTGLGSMD |
| 45540 | YFALNDITH |
| 45541 | YNLQWFKHY |
| 45542 | YSDNGRRFD |
| 45543 | YSIIWRVYR |
| 45544 | YSMDVMMSI |
| 45545 | YVHKPPDQG |
| 45546 | EVRSTRKNH |
| 45547 | LDGPIGMGT |
| 45548 | GAYSLHDWR |
| 45549 | IMVERWQHA |
| 45550 | MCDNFPSAH |
| 45551 | NGMDKPVDA |
| 45552 | AAWQSEIAN |
| 45553 | ACILDGFTL |
| 45554 | CAYNAPDWP |
| 45555 | EFTPEHRCW |
| 45556 | ETLKMYWEK |
| 45557 | GFSPISSPC |
| 45558 | GIDTISTSI |
| 45559 | GSEWKTFGD |
| 45560 | IMMCVWRSF |
| 45561 | KAFDNELHN |
| 45562 | KGEMEMSMA |
| 45563 | KKCDTMIGQ |
| 45564 | KMEHMWKMP |
| 45565 | MNGFCFGEA |
| 45566 | MVKQAQTMC |
| 45567 | NGKQFESGP |
| 45568 | NSASYCNPT |
| 45569 | NTLVNMESP |
| 45570 | TNSVMQNME |
| 45571 | TTGPKHCSV |
| 45572 | YSTYSEKSC |
| 45573 | YVETTKVWP |
| 45574 | TSERHSCMV |
| 45575 | ATAFKMLNT |
| 45576 | SPNGRGLCG |
| 45577 | AACWTVPQH |
| 45578 | AAESQGHRH |
| 45579 | AAHCIPNHD |
| 45580 | AAISSGFPA |
| 45581 | AAKVFPDGM |
| 45582 | AAMCCHLGV |
| 45583 | AAQCCTFGT |
| 45584 | AATASGYAS |
| 45585 | AAVFNDVEK |
| 45586 | AAVGINTRE |
| 45587 | AAVGSNEFA |
| 45588 | AAWMGNAVM |
| 45589 | ACAYSVASE |
| 45590 | ACCKAGFLV |
| 45591 | ACEYKVVNQ |
| 45592 | ACHNNVYHG |
| 45593 | ACKANTAQM |
| 45594 | ACKFTEISM |
| 45595 | ACLTKNWTR |
| 45596 | ADNVMHWWT |
| 45597 | ADSQSRVMP |
| 45598 | ADYHEGMFP |
| 45599 | AEHQHLACV |
| 45600 | AEMRTMGSQ |
| 45601 | AFAGTLQHP |
| 45602 | AFAWAHCPV |
| 45603 | AFMPQSWTQ |
| 45604 | AFMRNFDYP |
| 45605 | AFMSTGMLA |
| 45606 | AFMVTASNL |
| 45607 | AFNHWHYRN |
| 45608 | AFRSVVIKW |
| 45609 | AGCNRCWKQ |
| 45610 | AGDKWMASF |
| 45611 | AGGFWCEPF |
| 45612 | AGKWWWQYH |
| 45613 | AGVTLPVTL |
| 45614 | AHIAVFDCD |
| 45615 | AHLDMWWSN |
| 45616 | AHMCTQQSG |
| 45617 | AHMTEIAFA |
| 45618 | AHNMDVWCI |
| 45619 | AHPPTQYNH |
| 45620 | AHTLRQFWE |
| 45621 | AIACAHMGG |
| 45622 | AIEFHEAFT |
| 45623 | AILFLKGND |
| 45624 | AINGTGTFT |
| 45625 | AINWPMHIE |
| 45626 | AIQHPNQWC |
| 45627 | AIQNEENPG |
| 45628 | AIQTTETEE |
| 45629 | AISDKQPWR |
| 45630 | AITPCLEFK |
| 45631 | AIVEGVKHY |
| 45632 | AIVPIWTTN |
| 45633 | AIVQFDVEE |
| 45634 | AIVTASCPP |
| 45635 | AKCCSQLDY |
| 45636 | AKCYKEWWP |
| 45637 | AKDQYGLLS |
| 45638 | AKGLTHLTC |
| 45639 | AKLWRYRWI |
| 45640 | AKMLKDRAA |
| 45641 | AKMPYMSTH |
| 45642 | AKQMTAAQM |
| 45643 | AKSCEESRP |
| 45644 | AKSEFSVSI |
| 45645 | AKSGWTLME |
| 45646 | ALAHNFNGM |
| 45647 | ALDTFCRWP |
| 45648 | ALGLNSNHG |
| 45649 | ALMTWVRDC |
| 45650 | ALWYSKKEC |
| 45651 | AMASVRGGD |
| 45652 | AMENWYSYQ |
| 45653 | AMLDGMTSG |
| 45654 | AMLMSKEVA |
| 45655 | AMMQGVSNQ |
| 45656 | AMNVIASHF |
| 45657 | AMSCYTLWC |
| 45658 | AMSFTQFCS |
| 45659 | AMSPIVDAP |
| 45660 | AMSRLMMFS |
| 45661 | AMVEPQCQG |
| 45662 | AMWFCSVGD |
| 45663 | AMWRLNNVF |
| 45664 | ANCTQENQT |
| 45665 | ANECKQNVE |
| 45666 | ANHVTDTSC |
| 45667 | ANKMIVLNH |
| 45668 | ANKSVGEWR |
| 45669 | ANLFYSDRD |
| 45670 | ANLLGHYQA |
| 45671 | ANMLTNCDT |
| 45672 | ANMNCDKFS |
| 45673 | ANNVHCVKK |
| 45674 | ANNVTQAER |
| 45675 | ANQCHMGYT |
| 45676 | ANTRFPMKD |
| 45677 | APDTWVPVY |
| 45678 | APGNHNNWW |
| 45679 | APHCEKENG |
| 45680 | APISWQMAL |
| 45681 | APSMRKIIP |
| 45682 | APSNPRKVA |
| 45683 | AQAEKPCSY |
| 45684 | AQAKEWKTK |
| 45685 | AQCYKFNGH |
| 45686 | AQHTVWRFA |
| 45687 | AQLPFCQQD |
| 45688 | AQQKGGITF |
| 45689 | AQSAISVMV |
| 45690 | AQSAWHSWP |
| 45691 | ARLACCSAD |
| 45692 | ARMYYDDQP |
| 45693 | ARRLWQNDQ |
| 45694 | ARVQENHVG |
| 45695 | ASGMQGSWH |
| 45696 | ASIMQTKGI |
| 45697 | ASKPGIIFD |
| 45698 | ASMPFLSYT |
| 45699 | ASNILDLPE |
| 45700 | ASQWKIHGA |
| 45701 | ASSPFSQVR |
| 45702 | ASTCKKLAM |
| 45703 | ASYSILKQR |
| 45704 | ATAHIAPPS |
| 45705 | ATDTMQFIW |
| 45706 | ATIAQWCVH |
| 45707 | ATKLGCSRI |
| 45708 | ATLGHVMMC |
| 45709 | ATLTNQHAE |
| 45710 | ATMMKWDAN |
| 45711 | ATMWLEDQC |
| 45712 | ATRAMQVLE |
| 45713 | ATRLAFHQG |
| 45714 | ATRLITYAV |
| 45715 | ATSFTQFCS |
| 45716 | ATSGQTYSV |
| 45717 | ATSMCMHPR |
| 45718 | ATSPHLAWG |
| 45719 | ATVKFSDCV |
| 45720 | ATYKIDTSC |
| 45721 | AVAIRSWCG |
| 45722 | AVAMEFNVV |
| 45723 | AVDPMCFDE |
| 45724 | AVESICSTE |
| 45725 | AVFIPGQFW |
| 45726 | AVGMCQMDL |
| 45727 | AVLIAGAGH |
| 45728 | AVLKAEVVK |
| 45729 | AVPFPWGCC |
| 45730 | AVQLWWWHN |
| 45731 | AVRFYSTPI |
| 45732 | AVRWSQTPG |
| 45733 | AVTVVGTQI |
| 45734 | AVVAQCQSW |
| 45735 | AVVGWTAGQ |
| 45736 | AVYIMTQLQ |
| 45737 | AWAEPDMRW |
| 45738 | AWDAESCYK |
| 45739 | AWEKIHRSP |
| 45740 | AWITCLIAP |
| 45741 | AWMHDTCTK |
| 45742 | AWRSVASNH |
| 45743 | AWTHHFTLD |
| 45744 | AWWRVTHEQ |
| 45745 | AYAPLSHNN |
| 45746 | AYCEIPTNP |
| 45747 | AYDHVLESR |
| 45748 | AYEYRNHEF |
| 45749 | AYFPNSWGQ |
| 45750 | AYNRVWSCG |
| 45751 | AYPQCFQNP |
| 45752 | AYQAKYDLA |
| 45753 | AYTETSYLH |
| 45754 | AYTLCLCNE |
| 45755 | CANCPMFPT |
| 45756 | CAQFKMTYF |
| 45757 | CARQPMPPA |
| 45758 | CAYAIMPCE |
| 45759 | CCFQFELEC |
| 45760 | CCIFIWWLY |
| 45761 | CCSNKSRFV |
| 45762 | CCSPLCQQP |
| 45763 | CCVPMMNVC |
| 45764 | CCVTAWKNQ |
| 45765 | CDAIYVSCG |
| 45766 | CDCLFMLHQ |
| 45767 | CDNGYETNE |
| 45768 | CFCQGFRKR |
| 45769 | CFEHWADFE |
| 45770 | CFEKKQTDG |
| 45771 | CGEQKDQIG |
| 45772 | CGHLYNRDF |
| 45773 | CGLDYAMCQ |
| 45774 | CGRHWKEWE |
| 45775 | CGRQCKACF |
| 45776 | CHCQWMCQN |
| 45777 | CHGAHQKLE |
| 45778 | CHMPWHIHE |
| 45779 | CHMSTVMKV |
| 45780 | CHQETFLDA |
| 45781 | CHTGLMDAM |
| 45782 | CHVQFDGFG |
| 45783 | CICREGKFY |
| 45784 | CIMACQDRT |
| 45785 | CIMGMQWQF |
| 45786 | CIRHQNHYW |
| 45787 | CITAEQCGE |
| 45788 | CITPQFTSS |
| 45789 | CIVKRMECF |
| 45790 | CKAMTLAEM |
| 45791 | CKCYEMEVS |
| 45792 | CKEPHTMHE |
| 45793 | CKKPLGTRI |
| 45794 | CKNEYRIHT |
| 45795 | CKSVNPRMM |
| 45796 | CKVEWWHGN |
| 45797 | CKYEISSGL |
| 45798 | CLVSCPALN |
| 45799 | CLYVPLFAD |
| 45800 | CMEGGLMDW |
| 45801 | CMEMMKWAN |
| 45802 | CMFWANCSQ |
| 45803 | CMGSGFAEL |
| 45804 | CMKMWDCGK |
| 45805 | CMMPVLEEC |
| 45806 | CMRWFLLGS |
| 45807 | CNEVPSVMP |
| 45808 | CNSQKSYAH |
| 45809 | CNSWNCAYA |
| 45810 | CPDVGFQSG |
| 45811 | CPHLFVFDW |
| 45812 | CPPSNNPCE |
| 45813 | CPSRTWAIE |
| 45814 | CPTIIVKDW |
| 45815 | CPVANTTHP |
| 45816 | CQEFNSHTD |
| 45817 | CQNFSSLPW |
| 45818 | CQSVSANHI |
| 45819 | CRARRNPRL |
| 45820 | CRIRKRHIT |
| 45821 | CSCDEMEID |
| 45822 | CSDACQAMV |
| 45823 | CSDMLTKCN |
| 45824 | CSEHENLGI |
| 45825 | CSEKFNLHC |
| 45826 | CSETQVSTG |
| 45827 | CSQPHQMAS |
| 45828 | CSQRCMSFG |
| 45829 | CSRSDLLCG |
| 45830 | CSVWCIYYR |
| 45831 | CSYPAHRFH |
| 45832 | CTEVMAMGT |
| 45833 | CTFSSDKSD |
| 45834 | CTKLVNWYT |
| 45835 | CTLRCIMHT |
| 45836 | CTMRSGVGT |
| 45837 | CTPTILRGV |
| 45838 | CTQSQCHLC |
| 45839 | CTTEVRGTP |
| 45840 | CTTMKMNQA |
| 45841 | CTWFIQGPK |
| 45842 | CTYLAKLKC |
| 45843 | CVAAWGENG |
| 45844 | CVDNPFVLV |
| 45845 | CVFMGMMWD |
| 45846 | CVGRWWNFY |
| 45847 | CVHISDFGD |
| 45848 | CVIQGHSIE |
| 45849 | CVMAIESRG |
| 45850 | CVNRQHRIN |
| 45851 | CVPASGGWD |
| 45852 | CVTMKEFAM |
| 45853 | CWRQYVAGQ |
| 45854 | CYCTCEQPD |
| 45855 | CYELTMPLD |
| 45856 | DADRNNMSV |
| 45857 | DAEMLCYDY |
| 45858 | DAFWSNLHL |
| 45859 | DAHWGALSV |
| 45860 | DAIPKKWYG |
| 45861 | DALANNESR |
| 45862 | DALSMLRAP |
| 45863 | DALTNRASH |
| 45864 | DASMMWSRG |
| 45865 | DASYEYQSL |
| 45866 | DCAQTGEYG |
| 45867 | DCKNHYQSS |
| 45868 | DCMWYNRID |
| 45869 | DCPRSKMMT |
| 45870 | DCPTYYWRD |
| 45871 | DCSCHELAL |
| 45872 | DCYHICVTP |
| 45873 | DDELCPQCN |
| 45874 | DDHQSTWMN |
| 45875 | DDRCVQAAK |
| 45876 | DFAVSTSFD |
| 45877 | DFCMKEKAV |
| 45878 | DGCVHNQAI |
| 45879 | DGPICKGIY |
| 45880 | DGTHPCWNG |
| 45881 | DHCKTALPN |
| 45882 | DHERQRWLH |
| 45883 | DHIRQHTCS |
| 45884 | DHLKIRGAY |
| 45885 | DHQKRNVIQ |
| 45886 | DHQLVLVSG |
| 45887 | DHTVLFEGQ |
| 45888 | DHWITGPCR |
| 45889 | DHYLGHASY |
| 45890 | DICWKGWCF |
| 45891 | DIDRFTDVQ |
| 45892 | DIECKEIAI |
| 45893 | DIEITMQEN |
| 45894 | DIGPQDHWP |
| 45895 | DIHWMDHDF |
| 45896 | DIIEKVNEM |
| 45897 | DILERMNRA |
| 45898 | DINMNAQER |
| 45899 | DIRKDQHKN |
| 45900 | DIVIKKCNA |
| 45901 | DKASNQHNG |
| 45902 | DKFAAHALA |
| 45903 | DKGHHNSMH |
| 45904 | DKIDFSSVM |
| 45905 | DKKFGQFMS |
| 45906 | DKKWEEICA |
| 45907 | DKLELIGLD |
| 45908 | DKNFWVHWF |
| 45909 | DKTEMVIID |
| 45910 | DLASVGRWN |
| 45911 | DLCKMKEDK |
| 45912 | DLFRQERLF |
| 45913 | DLINWWHLQ |
| 45914 | DLKPWNPAW |
| 45915 | DLRLKVISR |
| 45916 | DLSRSFLAQ |
| 45917 | DLVIIYMFH |
| 45918 | DMELQFMYA |
| 45919 | DMGNYPCFE |
| 45920 | DMMLRHCVF |
| 45921 | DMRSNLPWH |
| 45922 | DMSHECVDA |
| 45923 | DMSHECVGA |
| 45924 | DMVDLEMYP |
| 45925 | DMWDEGDGD |
| 45926 | DNADMFNIG |
| 45927 | DNAKRPMTI |
| 45928 | DNENAIPMG |
| 45929 | DNGNMFQQH |
| 45930 | DNKLTEPCN |
| 45931 | DNKYYMSKC |
| 45932 | DNMEFESPG |
| 45933 | DNNAMLVCT |
| 45934 | DNPVTRCTD |
| 45935 | DNSWFVQGG |
| 45936 | DNVYTMHQW |
| 45937 | DPMYIPVNH |
| 45938 | DQCLCHTQA |
| 45939 | DQGLWENIP |
| 45940 | DQYFEGWRK |
| 45941 | DRCVMTDVL |
| 45942 | DRFMEFTGA |
| 45943 | DRFWSKYKD |
| 45944 | DRGMPTNWC |
| 45945 | DRGQSFQMF |
| 45946 | DRGTVNRDC |
| 45947 | DRSILHAHP |
| 45948 | DRTIIQNIP |
| 45949 | DRTSYPLHL |
| 45950 | DSAWYYWHP |
| 45951 | DSCHSRIIL |
| 45952 | DSELVRDYI |
| 45953 | DSIGLFRGG |
| 45954 | DSKIAHGRQ |
| 45955 | DSLRKYQCR |
| 45956 | DSLTVNMAH |
| 45957 | DSMAAHCAV |
| 45958 | DSNGIAHVP |
| 45959 | DSNKWAGLG |
| 45960 | DSNTHLPIG |
| 45961 | DSPQIVDVY |
| 45962 | DSQGINKLH |
| 45963 | DSRHSTFCL |
| 45964 | DSRHYANFM |
| 45965 | DSTPDRSIW |
| 45966 | DSTRIITQY |
| 45967 | DSYTMNWFY |
| 45968 | DTAAEVNMQ |
| 45969 | DTAEYRAGY |
| 45970 | DTDCARAIG |
| 45971 | DTDRQVKQS |
| 45972 | DTFEYTNAF |
| 45973 | DTKFYKIAY |
| 45974 | DTLQRACML |
| 45975 | DTLWYVNFA |
| 45976 | DTMMMPSSE |
| 45977 | DTMTTCYNS |
| 45978 | DTQNMKSSK |
| 45979 | DTRWWGMYA |
| 45980 | DTSSHECCD |
| 45981 | DTVHSGWQD |
| 45982 | DTVNQQKTW |
| 45983 | DVCKKGQCV |
| 45984 | DVCSQGQVP |
| 45985 | DVEMMIQRA |
| 45986 | DVEYYDHCF |
| 45987 | DVGYSAVWA |
| 45988 | DVKWGTSNL |
| 45989 | DVLPMEWCV |
| 45990 | DVLWGNRDS |
| 45991 | DVMAMAPSC |
| 45992 | DVNAGTRFP |
| 45993 | DVTDCKWGF |
| 45994 | DVWYPEFIG |
| 45995 | DWAHWAAYP |
| 45996 | DWCTDMTRK |
| 45997 | DWHNLVEYP |
| 45998 | DWLNLGRYM |
| 45999 | DYFREHMCP |
| 46000 | DYKLRARNA |
| 46001 | DYNGLSVPI |
| 46002 | DYYQWDGQI |
| 46003 | EADRWSSPN |
| 46004 | EAGRQAWTD |
| 46005 | EAHLDWIYP |
| 46006 | EAHWFTNYN |
| 46007 | EAKPWSGYL |
| 46008 | EAKRYHVDG |
| 46009 | EANMKEGLI |
| 46010 | EATIKALVF |
| 46011 | EATWTHADE |
| 46012 | ECDPRQMMC |
| 46013 | ECHQGSNME |
| 46014 | ECMDQQATW |
| 46015 | ECMQQCFVR |
| 46016 | ECQPFATVT |
| 46017 | ECVYNPANN |
| 46018 | EEKYKDAQW |
| 46019 | EEPGSQMTV |
| 46020 | EERPWKRDL |
| 46021 | EETEGIEPH |
| 46022 | EFDAAVIFK |
| 46023 | EFGYEHIVP |
| 46024 | EFRNSSCTN |
| 46025 | EGAHNKFDR |
| 46026 | EGCIPGGWS |
| 46027 | EGGSCNTQM |
| 46028 | EGNFIMGCS |
| 46029 | EGNQEGLNM |
| 46030 | EGNREGLNM |
| 46031 | EGNYMAGLG |
| 46032 | EGWHFPAME |
| 46033 | EHDGTPFNE |
| 46034 | EHDHQPSYA |
| 46035 | EHERFCQGR |
| 46036 | EHHRRGAFK |
| 46037 | EHMFNKDCT |
| 46038 | EHQRGHACN |
| 46039 | EHSYLLHDT |
| 46040 | EICSEINMK |
| 46041 | EIEQEEGGC |
| 46042 | EIGLYWIKC |
| 46043 | EIGYKQMYF |
| 46044 | EIKASHNYD |
| 46045 | EINQGDEDG |
| 46046 | EIQSVMPQP |
| 46047 | EISNLRQGF |
| 46048 | EISPHKILP |
| 46049 | EITGGHASW |
| 46050 | EIVWYFQDT |
| 46051 | EKAMRNGQA |
| 46052 | EKCCCLQWE |
| 46053 | EKEIKMLFG |
| 46054 | EKGPYWHQH |
| 46055 | EKHAMECVS |
| 46056 | EKIAPNHQA |
| 46057 | EKKMWVYTG |
| 46058 | EKLMAYIML |
| 46059 | EKLPIQMFN |
| 46060 | EKMPWLISN |
| 46061 | EKNWYQHIW |
| 46062 | EKQTRNQDG |
| 46063 | ELHHPIRRS |
| 46064 | ELMASNRCH |
| 46065 | ELMFVMHGT |
| 46066 | ELPMMNMAQ |
| 46067 | ELSFKTAQG |
| 46068 | ELTMANCMN |
| 46069 | EMERKNMFE |
| 46070 | EMGCDNKSF |
| 46071 | EMHLYHICH |
| 46072 | EMTSTMKSH |
| 46073 | EMYRALGHG |
| 46074 | ENGPMCDSN |
| 46075 | ENHSPNVDP |
| 46076 | ENILAIPAL |
| 46077 | ENKWQPKQA |
| 46078 | ENNSRQLIN |
| 46079 | EPCMRNLFG |
| 46080 | EPIRTEGAE |
| 46081 | EPLCHQIMP |
| 46082 | EPLVDEFGG |
| 46083 | EPRECFHIE |
| 46084 | EPWVMKLNK |
| 46085 | EQADYRAVQ |
| 46086 | EQERYEWDP |
| 46087 | EQGCQLVTT |
| 46088 | EQHPRMNFH |
| 46089 | EQIHQLRFG |
| 46090 | EQKWQWCQY |
| 46091 | EQQMLLQLS |
| 46092 | EQYMVNTRD |
| 46093 | ERCPWSVGE |
| 46094 | ERENSLWMS |
| 46095 | ERFLLEAVE |
| 46096 | ERGKWHALS |
| 46097 | ERQPFTMQW |
| 46098 | ERSWTHAHW |
| 46099 | ERTTKAEGY |
| 46100 | ERVARLQFT |
| 46101 | ESAKFVCEY |
| 46102 | ESCEDGIRT |
| 46103 | ESGFRKLRV |
| 46104 | ESHMHDHWI |
| 46105 | ESILVCATP |
| 46106 | ESIQLRSSQ |
| 46107 | ESSFVHEEQ |
| 46108 | ESTEPMSAT |
| 46109 | ESTLKTTYA |
| 46110 | ETCTEPKYW |
| 46111 | ETEMTSWQN |
| 46112 | ETKAVISAQ |
| 46113 | ETLERDWDY |
| 46114 | ETMKDVRFM |
| 46115 | ETNOLSGYC |
| 46116 | ETPKFNWSN |
| 46117 | ETRTDVPML |
| 46118 | ETTAAFKPG |
| 46119 | ETYDNNLQA |
| 46120 | ETYVCWFGS |
| 46121 | EVATSYARV |
| 46122 | EVDQAPMMI |
| 46123 | EVHRSELQR |
| 46124 | EVIGFLYHA |
| 46125 | EVMAMCDRY |
| 46126 | EVMPMCDRY |
| 46127 | EVQLTYLHE |
| 46128 | EVVTKHDSR |
| 46129 | EWCKQWMRF |
| 46130 | EWMTCGVSG |
| 46131 | EWSMLQYNH |
| 46132 | EWYIANISD |
| 46133 | EYELTQPAP |
| 46134 | EYIFENEMC |
| 46135 | EYLLCRGEA |
| 46136 | FADHFEAMF |
| 46137 | FAEHEIEHS |
| 46138 | FAIIRNDSH |
| 46139 | FAIMSSDHP |
| 46140 | FALNTEISA |
| 46141 | FAPFLKAGH |
| 46142 | FAQMNKDRC |
| 46143 | FARHPLKCA |
| 46144 | FASQHAVQA |
| 46145 | FCFWMPTTN |
| 46146 | FCGKLTHVG |
| 46147 | FDWRCGCWS |
| 46148 | FEQPPMHHQ |
| 46149 | FEVTMTVPE |
| 46150 | FFQRFLSAW |
| 46151 | FGAMTSDTI |
| 46152 | FGAYYNDRG |
| 46153 | FGETYNHSH |
| 46154 | FGEWITFYF |
| 46155 | FGHKIEAPH |
| 46156 | FGQHVWQGF |
| 46157 | FGRYFQFDL |
| 46158 | FGWWYKTFT |
| 46159 | FHAPTECMA |
| 46160 | FHFQFTMDK |
| 46161 | FHHLFDCNL |
| 46162 | FHLACIHAQ |
| 46163 | FHMTRYKMT |
| 46164 | FHRPSEGQE |
| 46165 | FIRLFMKLN |
| 46166 | FKECTNGMH |
| 46167 | FKMATAECP |
| 46168 | FKPCKDDDV |
| 46169 | FKVTEYELP |
| 46170 | FMDEIAVTM |
| 46171 | FMGFMGVMG |
| 46172 | FMQIWNKYS |
| 46173 | FMRMEMMGQ |
| 46174 | FMRPFLEPD |
| 46175 | FNFCFNHQF |
| 46176 | FNGIDQMRY |
| 46177 | FNMLLQVNR |
| 46178 | FNTTCQMSI |
| 46179 | FPICNQERI |
| 46180 | FPQPPCTYA |
| 46181 | FPTNONDPH |
| 46182 | FPTQMHYNA |
| 46183 | FQAKMHTLA |
| 46184 | FQDWPVTWC |
| 46185 | FQQQTGTRC |
| 46186 | FRFSTQHEA |
| 46187 | FRLNLLKTN |
| 46188 | FSEVTLQES |
| 46189 | FSKTGHCKF |
| 46190 | FSQCPITCC |
| 46191 | FSQGPGMHF |
| 46192 | FSQHQEHMR |
| 46193 | FSQRVEKTW |
| 46194 | FSSEAADYD |
| 46195 | FSTFTKPSP |
| 46196 | FSVPLGATT |
| 46197 | FSVSAQHSC |
| 46198 | FTFRVGEVN |
| 46199 | FTHGSVLHD |
| 46200 | FTMLGMEAI |
| 46201 | FTNPHAFYP |
| 46202 | FTQHIVGET |
| 46203 | FTTKYVYFD |
| 46204 | FVDHYYGHQ |
| 46205 | FVNHTRCNP |
| 46206 | FVRHDVIGQ |
| 46207 | FVTHWEYYP |
| 46208 | FVTMMQIAF |
| 46209 | FVTMRYEGH |
| 46210 | FWTSYTTNP |
| 46211 | FYGQNYWWY |
| 46212 | FYPRSWPQA |
| 46213 | GAARYGYNI |
| 46214 | GADSSGGWN |
| 46215 | GAFIRDFDI |
| 46216 | GAGSVQEIH |
| 46217 | GAGTTMRPD |
| 46218 | GAHWWGGCV |
| 46219 | GAIPLLNWL |
| 46220 | GAKMQIAFW |
| 46221 | GAKQHMPCY |
| 46222 | GAVNSEVCE |
| 46223 | GCCWSYYCG |
| 46224 | GCDTTKFDA |
| 46225 | GCFIYWKDS |
| 46226 | GCVKDRCWM |
| 46227 | GCWNERCON |
| 46228 | GCYWACWLT |
| 46229 | GDNYTAWVG |
| 46230 | GEFMMFDRG |
| 46231 | GEGWNDDLG |
| 46232 | GEREYWNWE |
| 46233 | GFACLDHFD |
| 46234 | GFDIVLKDF |
| 46235 | GFELNMRIS |
| 46236 | GFGNTFCIT |
| 46237 | GFGTVSMER |
| 46238 | GFGYMWCKG |
| 46239 | GFLLTYGED |
| 46240 | GFMIGNQYL |
| 46241 | GFRVIISNW |
| 46242 | GFSCLICFD |
| 46243 | GFTQTCMTN |
| 46244 | GGLLYIECY |
| 46245 | GGTIDGHVG |
| 46246 | GGVKFNNWW |
| 46247 | GHMFVNIDQ |
| 46248 | GHMMMMECW |
| 46249 | GHSLTMRAH |
| 46250 | GHTARDIHA |
| 46251 | GHTHSQPAA |
| 46252 | GHTPGGSFY |
| 46253 | GIANTSDSY |
| 46254 | GICCILWDR |
| 46255 | GIDSMVIQR |
| 46256 | GIHEIMVQR |
| 46257 | GIHMVTAMS |
| 46258 | GIKFTQQDL |
| 46259 | GILRQIEKP |
| 46260 | GISICGQNW |
| 46261 | GKDTPNSFP |
| 46262 | GKGEVLAVA |
| 46263 | GKGTMEFGN |
| 46264 | GKGYGQHDG |
| 46265 | GKILWMSAR |
| 46266 | GKMQVLHSA |
| 46267 | GKNMLIDGG |
| 46268 | GKPYTQIIR |
| 46269 | GKSTCKRDV |
| 46270 | GLEMRNQAM |
| 46271 | GLFQQAMQG |
| 46272 | GLGCLMQGN |
| 46273 | GLSVGQRIE |
| 46274 | GLYERLACC |
| 46275 | GMHYSQCQH |
| 46276 | GMKAVFIGD |
| 46277 | GMWVNHGAK |
| 46278 | GNEHCVKYA |
| 46279 | GNEKIERYD |
| 46280 | GNEMPNYGG |
| 46281 | GNHVNYNMH |
| 46282 | GPEKLTWGP |
| 46283 | GPPHYRNPE |
| 46284 | GQFIFDEFV |
| 46285 | GQGFMLWNG |
| 46286 | GQGKNWVSW |
| 46287 | GQGMCDRKT |
| 46288 | GQLNCMKGS |
| 46289 | GQQENPVFT |
| 46290 | GQTSPLMYP |
| 46291 | GRAPMCAAA |
| 46292 | GRLVVHQFK |
| 46293 | GRNMFLWWW |
| 46294 | GRSLSQSEW |
| 46295 | GRYQSSQFC |
| 46296 | GSASHNVWY |
| 46297 | GSDYWLCDK |
| 46298 | GSFYAQYEE |
| 46299 | GSFYCQWNN |
| 46300 | GSGKMWQED |
| 46301 | GSLLFNPEE |
| 46302 | GSMKEMMMR |
| 46303 | GSMVASSWC |
| 46304 | GSNQRDCSR |
| 46305 | GSRPTPADW |
| 46306 | GSSHYMEFY |
| 46307 | GSYTKPTNV |
| 46308 | GTAHNVFMG |
| 46309 | GTCLDQTGC |
| 46310 | GTETHCDFE |
| 46311 | GTGAFTQVP |
| 46312 | GTIAFSDNN |
| 46313 | GTIPMNCFW |
| 46314 | GTKMFQESI |
| 46315 | GTMFWGWTG |
| 46316 | GTNNIINVR |
| 46317 | GTQHFWMDH |
| 46318 | GTRWIHDMA |
| 46319 | GTSQEKRGQ |
| 46320 | GVAAVGLYK |
| 46321 | GVIVPISRS |
| 46322 | GVLKQANQQ |
| 46323 | GVQNTALPR |
| 46324 | GVTSPVGTC |
| 46325 | GVVDNQQCL |
| 46326 | GVWPHYENK |
| 46327 | GWCITKARN |
| 46328 | GWDHFMQNT |
| 46329 | GWMDYIAAH |
| 46330 | GWYNWMIQY |
| 46331 | GYESKNTWY |
| 46332 | GYEWVQEIE |
| 46333 | GYFPNHWCK |
| 46334 | GYHCTGAHS |
| 46335 | GYICEGTCH |
| 46336 | GYNERFEHK |
| 46337 | GYNPMMSAA |
| 46338 | GYSQKPRTP |
| 46339 | GYTYNQQVC |
| 46340 | HADAENQVM |
| 46341 | HAIPHTMWH |
| 46342 | HAIQEGTVP |
| 46343 | HAIYWIPQV |
| 46344 | HAMATECGP |
| 46345 | HAMLSHGCC |
| 46346 | HANESISMT |
| 46347 | HASYQSSFG |
| 46348 | HATSQELCQ |
| 46349 | HATTFEWDS |
| 46350 | HAYALAVEH |
| 46351 | HCCSANNSP |
| 46352 | HCELISRIA |
| 46353 | HCELLPVNE |
| 46354 | HCFMAHCSY |
| 46355 | HCMNWMING |
| 46356 | HCQILPGWA |
| 46357 | HCVNEHAWE |
| 46358 | HDGFPWADE |
| 46359 | HERNWPFAC |
| 46360 | HESCLKLAG |
| 46361 | HEWAGELQT |
| 46362 | HEYGFGEAR |
| 46363 | HFANICVRG |
| 46364 | HFEHLWCQS |
| 46365 | HFGHYLGMF |
| 46366 | HFISHDYHN |
| 46367 | HFMPNNFLA |
| 46368 | HFVNSYCWW |
| 46369 | HGAQFARCS |
| 46370 | HGNEVDHSM |
| 46371 | HHAVMNKMD |
| 46372 | HHEVTTKFQ |
| 46373 | HHFHSGCCW |
| 46374 | HHPPELDRM |
| 46375 | HHTDRNGGD |
| 46376 | HHVLKGDMA |
| 46377 | HIDGATVSG |
| 46378 | HIKWDCWCP |
| 46379 | HINAFDNIR |
| 46380 | HISWWPACG |
| 46381 | HITTLYMEA |
| 46382 | HKLVAHERH |
| 46383 | HKPMCDRVF |
| 46384 | HLMGLCAMS |
| 46385 | HLQSISHCH |
| 46386 | HLVGMSLTQ |
| 46387 | HMEMPYKLG |
| 46388 | HMKCDVWQP |
| 46389 | HMLKKEDMN |
| 46390 | HMTTVNLQY |
| 46391 | HNGKSVLEW |
| 46392 | HNKMTYNYE |
| 46393 | HNNMFFWDR |
| 46394 | HNRAHENDK |
| 46395 | HNSVVNDHE |
| 46396 | HNTQNGFAD |
| 46397 | HNWYKRIVQ |
| 46398 | HPGIDLVCG |
| 46399 | HQCFWIYGS |
| 46400 | HQFQFYQWM |
| 46401 | HQPPIMNQG |
| 46402 | HRCTMQPDI |
| 46403 | HRCTPYNRD |
| 46404 | HRDHDITDF |
| 46405 | HRFSPPMET |
| 46406 | HRHFEMSVI |
| 46407 | HRMVIRKSQ |
| 46408 | HRVQCDEAP |
| 46409 | HSCTSGNPG |
| 46410 | HSESIDVWS |
| 46411 | HSETQRHQM |
| 46412 | HSGPWSNCE |
| 46413 | HSMQTKFDW |
| 46414 | HSSALFDME |
| 46415 | HSTQAGGMY |
| 46416 | HSTTACVHN |
| 46417 | HSVMWGKEN |
| 46418 | HTDKWWRDI |
| 46419 | HTEKGGEQA |
| 46420 | HTIHGAGLI |
| 46421 | HTIPVPMAA |
| 46422 | HTNTCLFEE |
| 46423 | HTQIIGGSD |
| 46424 | HTQWFNMGE |
| 46425 | HVDQYYRSA |
| 46426 | HVDTEKGRE |
| 46427 | HVELSQLHS |
| 46428 | HVEWVNAEK |
| 46429 | HVLPVCYRM |
| 46430 | HVMFPWGMW |
| 46431 | HVMYQHWMM |
| 46432 | HVNIEHQGT |
| 46433 | HVSDCCKSQ |
| 46434 | HVVKYPLSG |
| 46435 | HVYRGNGME |
| 46436 | HWDECNYVG |
| 46437 | HWDQFNGQF |

### Example 46

Treatment of Spinal Muscular Atrophy (SMA) with an AAV5-Derived Virion Encapsidating a Therapeutic Payload

This example describes treatment of spinal muscular atrophy (SMA) with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a region from a position corresponding to 581 to a position corresponding to 589 in the VP1 capsid polypeptide and where the polynucleotide sequence encoding for the variant AAV5 Cap encodes for an amino acid sequence of any one of SEQ ID NO: 34991-SEQ ID NO: 37437. The therapeutic payload is a guide RNA targeting an mRNA encoded for by a gene implicated in SMA or a transgene. The mRNA targeted by the guide RNA is an mRNA encoded for by a gene encoding for survival motor neuron (SMN) protein. The transgene is SMN. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced spinal cord tropism as compared to wildtype AAV5 virions encapsidating the therapeutic payload. Upon administration to the subject, a lower dose of the variant AAV5 virions encapsidating the therapeutic payload is administered as compared to wildtype AAV5 virions encapsidating the therapeutic payload. Upon administration to the subject, at least one symptom of SMA is alleviated or the subject is cured.

1. CERTAIN SEQUENCES
>AAV5
VP1, from Chiorini et al., J. Virol.
73(2):1309-1319 (1999)
[SEQ ID NO: 1]
MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGP
LGDNNQGADGVGNASGDWHCDSTWMGDRVVTKSTRTWVLPSYNNHQYREI
KSGSVDGSNANAYFGYSTPWGYFDENRFHSHWSPRDWQRLINNYWGERPR
SLRVKIFNIQVKEVTVQDSTTTIANNLTSTVQVFTDDDYQLPYVVGNGTE
GCLPAFPPQVFTLPQYGYATLNRDNTENPTERSSFFCLEYFPSKMLRTGN
NFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGA
SYQVPPQPNGMTNNLQGSNTYALENTMIFNSQPANPGTTATYLEGNMLIT
SESETQPVNRVAYNVGGQMATNNQSSTTAPATGTYNLQEIVPGSVWMERD
VYLQGPIWAKIPETGAHFHPSPAMGGFGLKHPPPMMLIKNTPVPGNITSF
SDVPVSSFITQYSTGQVTVEMEWELKKENSKRWNPEIQYTNNYNDPQFVD
FAPDSTGEYRTTRPIGTRYLTRPL
>AAV5 Library scaffold
[SEQ ID NO: 2]
MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGP
LGDNNQGADGVGNASGDWHCDSTWMGDRVVTKSTRTWVLPSYNNHQYREI
KSGSVDGSNANAYFGYSTPWGYFDENRFHSHWSPRDWQRLINNYWGFRPR
SLRVKIFNIQVKEVTVQDSTTTIANNLTSTVQVFTDDDYQLPYVVGNGTE
GCLPAFPPQVFTLPQYGYATLNRDNTENPTERSSFFCLEYFPSKMLRTGN
NFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGA
SYQVPPQPNGMTNNLQGSNTYALENTMIFNSQPANPGTTATYLEGNMLIT
SESETQPVNRVAYNVGGQMATNNQSSTTAP Xaa1, Xaa2, Xaa3,
Xaa4, Xaa5, Xaa6, Xaa7, Xaa8, Xaa9 IVPGSVWMERDVYLQ
GPIWAKIPETGAHFHPSPAMGGFGLKHPPPMMLIKNTPVPGNITSFSDVP
VSSFITQYSTGQVTVEMEWELKKENSKRWNPEIQYTNNYNDPQFVDFAPD
STGEYRTTRPIGTRYLTRPL Xaal, Xaa2, Xaa3, Xaa4, Xaa5,
Xaa6, Xaa7, Xaa8, and Xaa9 are each independently
selected from A, R, N, D, C, E, Q, G, H, I, L, K,
M, F, P, S, T, W, Y, and V
SEQ ID NO: 3
MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGP
LGDNNQGADGVGNASGDWHCDSTWMGDRVVTKSTRTWVLPSYNNHQYREI
KSGSVDGSNANAYFGYSTPWGYFDFNRFHSHWSPRDWQRLINNYWGFRPR
SLRVKIFNIQVKEVTVQDSTTTIANNLTSTVQVFTDDDYQLPYVVGNGTE
GCLPAFPPQVFTLPQYGYATLNRDNTENPTERSSFFCLEYFPSKMLRTGN
NFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGA
SYQVPPQPNGMTNNLQGSNTYALENTMIFNSQPANPGTTATYLEGNMLIT
SESETQPVNRVAYNVGGQMATNNQSSTTAPATGTXaa5NLQEIVPGSVWM
ERDVYLOGPIWAKIPETGAHFHPSPAMGGFGLKHPPPMMLIKNTPVPGNI
TSFSDVPVSSFITQYSTGQVTVEMEWELKKENSKRWNPEIQYTNNYNDPQ
FVDFAPDSTGEYRTTRPIGTRYLTRPL Wherein Xaa5 is V
(Y585V)
SEQ ID NO: 4
MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGP
LGDNNQGADGVGNASGDWHCDSTWMGDRVVTKSTRTWVLPSYNNHQYREI
KSGSVDGSNANAYFGYSTPWGYFDFNRFHSHWSPRDWQRLINNYWGFRPR
SLRVKIFNIQVKEVTVQDSTTTIANNLTSTVQVFTDDDYQLPYVVGNGTE
GCLPAFPPQVFTLPQYGYATLNRDNTENPTERSSFFCLEYFPSKMLRTGN
NFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGA
SYQVPPQPNGMTNNLQGSNTYALENTMIFNSQPANPGTTATYLEGNMLIT
SESETQPVNRVAYNVGGQMATNNQSSTTAPATGTYNXaa7QEIVPGSVWM
ERDVYLQGPIWAKIPETGAHFHPSPAMGGFGLKHPPPMMLIKNTPVPGNI
TSFSDVPVSSFITQYSTGQVTVEMEWELKKENSKRWNPEIQYTNNYNDPQ
FVDFAPDSTGEYRTTRPIGTRYLTRPL Wherein Xaa7 is T
(L587T)
SEQ ID NO: 5
MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGP
LGDNNQGADGVGNASGDWHCDSTWMGDRVVTKSTRTWVLPSYNNHQYREI
KSGSVDGSNANAYFGYSTPWGYFDENRFHSHWSPRDWQRLINNYWGFRPR
SLRVKIFNIQVKEVTVQDSTTTIANNLTSTVQVFTDDDYQLPYVVGNGTE
GCLPAFPPQVFTLPQYGYATLNRDNTENPTERSSFFCLEYFPSKMLRTGN
NFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGA
SYQVPPQPNGMTNNLQGSNTYALENTMIFNSQPANPGTTATYLEGNMLIT
SESETQPVNRVAYNVGGQMATNNQSSTTAPXaa1TGTYNLQEIVPGSVWM
ERDVYLQGPIWAKIPETGAHFHPSPAMGGFGLKHPPPMMLIKNTPVPGNI
TSFSDVPVSSFITQYSTGQVTVEMEWELKKENSKRWNPEIQYTNNYNDPQ
FVDFAPDSTGEYRTTRPIGTRYLTRPL Wherein Xaa1 is T
(A581T)
SEQ ID NO: 6
MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGP
LGDNNQGADGVGNASGDWHCDSTWMGDRVVTKSTRTWVLPSYNNHQYREI
KSGSVDGSNANAYFGYSTPWGYFDENRFHSHWSPRDWQRLINNYWGFRPR
SLRVKIFNIQVKEVTVQDSTTTIANNLTSTVQVFTDDDYQLPYVVGNGTE
GCLPAFPPQVFTLPQYGYATLNRDNTENPTERSSFFCLEYFPSKMLRTGN
NFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGA
SYQVPPQPNGMTNNLQGSNTYALENTMIFNSQPANPGTTATYLEGNMLIT
SESETQPVNRVAYNVGGQMATNNQSSTTAPAXaaGTYNLQEIVPGSVWME
RDVYLQGPIWAKIPETGAHFHPSPAMGGFGLKHPPPMMLIKNTPVPGNIT
SFSDVPVSSFITQYSTGQVTVEMEWELKKENSKRWNPEIQYTNNYNDPQF
VDFAPDSTGEYRTTRPIGTRYLTRPL Wherein Xaa2 is A
(T582A)
SEQ ID NO: 7
MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGP
LGDNNQGADGVGNASGDWHCDSTWMGDRVVTKSTRTWVLPSYNNHQYREI
KSGSVDGSNANAYFGYSTPWGYFDFNRFHSHWSPRDWQRLINNYWGFRPR
SLRVKIFNIQVKEVTVQDSTTTIANNLTSTVQVFTDDDYQLPYVVGNGTE
GCLPAFPPQVFTLPQYGYATLNRDNTENPTERSSFFCLEYFPSKMLRTGN
NFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGA
SYQVPPQPNGMTNNLQGSNTYALENTMIFNSQPANPGTTATYLEGNMLIT
SESETQPVNRVAYNVGGQMATNNQSSTTAPATGXaa4YNLQEIVPGSVWM
ERDVYLQGPIWAKIPETGAHFHPSPAMGGFGLKHPPPMMLIKNTPVPGNI
TSFSDVPVSSFITQYSTGQVTVEMEWELKKENSKRWNPEIQYTNNYNDPQ
FVDFAPDSTGEYRTTRPIGTRYLTRPL Wherein Xaa4 is A
(T584A)
SEQ ID NO: 8
MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGP
LGDNNQGADGVGNASGDWHCDSTWMGDRVVTKSTRTWVLPSYNNHQYREI
KSGSVDGSNANAYFGYSTPWGYFDFNRFHSHWSPRDWQRLINNYWGERPR
SLRVKIFNIQVKEVTVQDSTTTIANNLTSTVQVFTDDDYQLPYVVGNGTE
GCLPAFPPQVFTLPQYGYATLNRDNTENPTERSSFFCLEYFPSKMLRTGN
NFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGA
SYQVPPQPNGMTNNLQGSNTYALENTMIENSQPANPGTTATYLEGNMLIT
SESETQPVNRVAYNVGGQMATNNQSSTTAPATGTXaa5NXaa7QEIVPGS
VWMERDVYLOGPIWAKIPETGAHFHPSPAMGGFGLKHPPPMMLIKNTPVP
GNITSFSDVPVSSFITQYSTGQVTVEMEWELKKENSKRWNPEIQYTNNYN
DPQFVDFAPDSTGEYRTTRPIGTRYLTRPL Wherein Xaa5 is V
(Y585V) and Xaa7 is T (L587T)
SEQ ID NO: 9
ATGTYNLQE (581-589 sialic acid binding region of
AAV5 VP1 capsid and reference for Xaal-Xaa9)
(FIG. 10, Middle Variant DNA)
SEQ ID NO: 111
AATCCAGCTATGTTCAATTGCGATTAC
(FIG. 10, Middle Variant; amino acid)
SEQ ID NO: 112
NPAMFNCDY
(FIG. 10, Right Variant, DNA)
SEQ ID NO: 113
AAGTTGACCGCCCACATCTACGCCTTG
(FIG. 10, Right Variant; amino acid)
SEQ ID NO: 114
KLTAHIYAL
SEQ ID NO: 115-1114
(TABLE 8, wherein the listed AAs represent
Xaa1-Xaa9 of SEQ ID NO: 2)
AAV5 VP2 sequence
(SEQ ID NO: 1115)
TAPTGKRIDDHFPKRKKARTEEDSKPSTSSDAEAGPSGSQQLQIPAQPAS
SLGADTMSAGGGGPLGDNNQGADGVGNASGDWHCDSTWMGDRVVTKSTRT
WVLPSYNNHQYREIKSGSVDGSNANAYFGYSTPWGYFDFNRFHSHWSPRD
WQRLINNYWGFRPRSLRVKIFNIQVKEVTVQDSTTTIANNLTSTVQVFTD
DDYQLPYVVGNGTEGCLPAFPPQVFTLPQYGYATLNRDNTENPTERSSFF
CLEYFPSKMLRTGNNFEFTYNFEEVPFHSSFAPSQNLFKLANPLVDQYLY
RFVSTNNTGGVQFNKNLAGRYANTYKNWFPGPMGRTQGWNLGSGVNRASV
SAFATTNRMELEGASYQVPPQPNGMTNNLQGSNTYALENTMIFNSQPANP
GTTATYLEGNMLITSESETQPVNRVAYNVGGQMATNNQSSTTAPATGTYN
LQEIVPGSVWMERDVYLQGPIWAKIPETGAHFHPSPAMGGFGLKHPPPMM
LIKNTPVPGNITSFSDVPVSSFITQYSTGQVTVEMEWELKKENSKRWNPE
IQYTNNYNDPQFVDFAPDSTGEYRTTRPIGTRYLTRPL
AAV5 VP3 sequence
(SEQ ID NO: 1116)
MSAGGGGPLGDNNQGADGVGNASGDWHCDSTWMGDRVVTKSTRTWVLPSY
NNHQYREIKSGSVDGSNANAYFGYSTPWGYFDFNRFHSHWSPRDWQRLIN
NYWGFRPRSLRVKIFNIQVKEVTVQDSTTTIANNLTSTVQVFTDDDYQLP
YVVGNGTEGCLPAFPPQVFTLPQYGYATLNRDNTENPTERSSFFCLEYFP
SKMLRTGNNFEFTYNFEEVPFHSSFAPSQNLFKLANPLVDQYLYRFVSTN
NTGGVQFNKNLAGRYANTYKNWFPGPMGRTQGWNLGSGVNRASVSAFATT
NRMELEGASYQVPPQPNGMTNNLQGSNTYALENTMIFNSQPANPGTTATY
LEGNMLITSESETQPVNRVAYNVGGQMATNNQSSTTAPATGTYNLQEIVP
GSVWMERDVYLQGPIWAKIPETGAHFHPSPAMGGFGLKHPPPMMLIKNTP
VPGNITSFSDVPVSSFITQYSTGQVTVEMEWELKKENSKRWNPEIQYTNN
YNDPQFVDFAPDSTGEYRTTRPIGTRYLTRPL

### 2. EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57 (b) (1), to relate to each and every individual publication, database entry (e.g., Genbank sequences or GeneID entries), patent application, or patent, each of which is clearly identified in compliance with 37 C.F.R. § 1.57 (b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

1-87. (canceled)

88. An engineered adeno-associated virus 9 (AAV9) viral protein (VP) capsid polypeptide comprising a region corresponding to residues 581-589 of SEQ ID NO: 2 and having a sequence of Xaa1, Xaa2, Xaa3, Xaa4, Xaa5, Xaa6, Xaa7, Xaa8, Xaa9, wherein:
a) Xaa1 is selected from A, R, N, D, C, E, Q, G, H, I, L, K, M, F, P, S, T, W, Y, or V;
b) Xaa2 is selected from A, C, I, K, S, T, or V;
c) Xaa3 is selected from A, G, I, K, M, Q, R, S, T, or V;
d) Xaa4 is selected from A, I, K, L, P, Q, R, S, T, or V;
e) Xaa5 is selected from F, I, L, M, T, V, or Y;
f) Xaa6 is selected from F, H, M, N, Q, S, or Y;
g) Xaa7 is selected from A, C, K, M, Q or S;
h) Xaa8 is selected from A, C, F, G, M, Q, or S; and
i) Xaa9 is selected from A, R, N, D, C, E, Q, G, H, I, L, K, M, F, P, S, T, W, Y, or V.

89. The engineered adeno-associated virus 9 (AAV9) viral protein (VP) capsid polypeptide of claim 88, wherein Xaa5 is selected from F, L, or Y.

90. The engineered adeno-associated virus 9 (AAV9) viral protein (VP) capsid polypeptide of claim 88, wherein Xaa7 is selected from A, C, or S.

91. The engineered adeno-associated virus 9 (AAV9) viral protein (VP) capsid polypeptide of claim 88, wherein Xaa7 is S.

92. An engineered adeno-associated virus (AAV) viral protein (VP) capsid polypeptide having an amino acid sequence of SEQ ID NO: 2, wherein:
a) Xaa1 is selected from A, C, K, M, Q, R, T, or W, Xaa4 is selected from E, G, I, M, Q, or R, and Xaa5 is selected from C, G, K, I, M, or R;
b) Xaa3 is selected from A, R, or W, and Xaa6 is selected from I, K, L, P, Q, R, Y;
c) Xaa1 is selected from A, C, K, M, Q, R, T, or W, Xaa3 is selected from A, H, N, R, or W, Xaa6 is R, and Xaa8 is selected from C, G, H, K, L, or V;
d) Xaa1 is selected from A, C, K, M, Q, R, T, or W, Xaa3 is selected from A, H, N, R, or W, and Xaa5 is I;
e) Xaa3 is R, Xaa6 is R, and Xaa8 is selected from C, G, H, K, L, or V;
f) Xaa3 is selected from A, R, or W, Xaa5 is selected from K, I, or R, and Xaa6 is selected from I, K, L, P, Q, R, Y;
g) Xaa2 is selected from F, I, K, R, T, or W, Xaa6 is selected from I, K, L, P, Q, R, Y, and Xaa8 is H;
h) Xaa1 is selected from K, Q, R, or W, Xaa2 is selected from F, I, R or T, Xaa3 is selected from A, R, or W, Xaa4 is selected from E, M, or R, Xaa6 is selected from K, R, or Y, Xaa7 is selected from 1, R, or V, and Xaa8 is H;
i) Xaa6 is selected from I, K, L, P, Q, R, Y, Xaa8 is H, and Xaa9 is selected from I, K, L, R, or V;
j) Xaa1 is selected from A, C, K, M, Q, R, T, or W, Xaa4 is R, Xaa7 is selected from I, R, or V, and Xaa8 is selected from C, G, H, K, L, or V;
k) Xaa3 is selected from A, R, or W, Xaa6 is selected from I, K, L, P, Q, R, Y, Xaa7 is selected from D, I, K, R, V, or W;
l) Xaa1 is selected from K, Q, R, or W, Xaa4 is selected from E, M, or R, and Xaa7 is selected from I, R, or V;
m) Xaa3 is selected from A, R, or W, and Xaa4 is selected from E, M, or R;
n) Xaa1 is selected from A, C, K, M, Q, R, T, or W, and Xaa8 is selected from C, G, H, K, L, or V;
o) Xaa1 is selected from A, C, K, M, Q, R, T, or W, Xaa3 is selected from A, R, or W, Xaa6 is selected from K, R, or Y, and Xaa7 is selected from D, I, K, R, V, or W;
p) Xaa3 is selected from A, R, or W, and Xaa6 is selected from I, K, L, P, Q, R, Y;
q) Xaa1 is selected from A, C, K, M, Q, R, T, or W, and Xaa4 is R;
r) Xaa1 is selected from K, Q, R, or W, and Xaa2 is selected from F, I, K, R, T, or W; or
s) Xaa3 is selected from A, R, or W, Xaa4 is selected from E, G, I, M, Q, or R, Xaa5 is selected from C, G, K, I, M, or R, Xaa6 is selected from I, K, L, P, Q, R, Y, and Xaa8 is selected from C, G, H, K, L, or V;
wherein the engineered AAV VP capsid polypeptide is capable of assembling into a recombinant AAV virion (rAAV), and
wherein the engineered AAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8.

93. An engineered adeno-associated virus (AAV) viral protein (VP) capsid polypeptide having an amino acid sequence at least 70% identical to SEQ ID NO: 1,
wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive,
wherein the region from the residue corresponding to residue 581 to the residue corresponding to residue 589, inclusive, has a sequence that is:
a) at least 50% identical to SEQ ID NO: 18831;
b) at least 50% identical to SEQ ID NO: 7557 or SEQ ID NO: 3006; or
c) at least 50% identical to any one of SEQ ID NO: 8946, SEQ ID NO: 16723, SEQ ID NO: 16834, SEQ ID NO: 17680, SEQ ID NO: 24842, SEQ ID NO: 25086, SEQ ID NO: 25927, SEQ ID NO: 47145, SEQ ID NO: 19284, SEQ ID NO: 28511, SEQ ID NO: 31089, SEQ ID NO: 32199, SEQ ID NO: 37500, SEQ ID NO: 42502, SEQ ID NO: 19640, SEQ ID NO: 26282, SEQ ID NO: 6640, SEQ ID NO: 22254, SEQ ID NO: 25523, SEQ ID NO: 32181, SEQ ID NO: 43670, SEQ ID NO: 15106, SEQ ID NO: 15630, SEQ ID NO: 45792, SEQ ID NO: 3221, SEQ ID NO: 12881, SEQ ID NO: 39889, SEQ ID NO: 45224, SEQ ID NO: 1817, SEQ ID NO: 17007, SEQ ID NO: 25557, SEQ ID NO: 43597, SEQ ID NO: 3119, SEQ ID NO: 7741, SEQ ID NO: 22974, SEQ ID NO: 23042, SEQ ID NO: 23937, SEQ ID NO: 39439, SEQ ID NO: 7606, SEQ ID NO: 18202, SEQ ID NO: 33959, SEQ ID NO: 34900, SEQ ID NO: 20187, SEQ ID NO: 21490, SEQ ID NO: 25677, SEQ ID NO: 26313, SEQ ID NO: 32448, SEQ ID NO: 41518, SEQ ID NO: 15329, SEQ ID NO: 34800, SEQ ID NO: 3408, SEQ ID NO: 9753, SEQ ID NO: 4211, SEQ ID NO: 26119, SEQ ID NO: 34958, SEQ ID NO: 36613, SEQ ID NO: 42984, SEQ ID NO: 4562, SEQ ID NO: 9483, SEQ ID NO: 12205, SEQ ID NO: 22243, SEQ ID NO: 31272, SEQ ID NO: 42602, SEQ ID NO: 8966, SEQ ID NO: 9863, SEQ ID NO: 25444, SEQ ID NO: 28758, SEQ ID NO: 5315, SEQ ID NO: 12449, SEQ ID NO: 21630, SEQ ID NO: 39246, SEQ ID NO: 19156, SEQ ID NO: 38663, SEQ ID NO: 25922, SEQ ID NO: 43034, SEQ ID NO: 6837, SEQ ID NO: 21839, or SEQ ID NO: 21785;
wherein the engineered AAV VP capsid polypeptide is capable of assembling into a recombinant AAV virion (rAAV),
wherein the at least one mutation confers higher tropism for a central nervous system (CNS) tissue on the rAAV as compared to an rAAV virion having a wildtype AAV5 VP capsid polypeptide of SEQ ID NO: 1, and
wherein the engineered AAV VP capsid polypeptide does not have the sequence of any of SEQ ID NO: 3, SEQ ID NO: 4, SEQ ID NO: 5, SEQ ID NO: 6, SEQ ID NO: 7, and SEQ ID NO: 8.

94. The AAV VP capsid polypeptide of claim 93, wherein the sequence is:
a) at least 60% identical to SEQ ID NO: 18831; or
b) at least 60% identical to SEQ ID NO: 7557 or SEQ ID NO: 3006.

95. The AAV VP capsid polypeptide of claim 93, wherein the sequence is SEQ ID NO: 18831.

96. The engineered AAV VP capsid polypeptide of claim 93, wherein the engineered AAV VP capsid polypeptide has at least one mutation as compared to SEQ ID NO: 1 in the region from a residue corresponding to residue 581 of SEQ ID NO: 1 to a residue corresponding to residue 589 of SEQ ID NO: 1, inclusive, wherein the engineered AAV VP capsid polypeptide is capable of assembling into a recombinant AAV virion (rAAV), and wherein the at least one mutation confers higher tropism for a central nervous system (CNS) tissue on the rAAV as compared to an rAAV virion having a wildtype AAV5 VP capsid polypeptide of SEQ ID NO: 1.

97. The engineered AAV VP capsid polypeptide of claim 96, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.

98. The engineered AAV VP capsid polypeptide of claim 96, wherein tropism for CNS tissue is measured as a relative accumulation of the rAAV virion in a CNS tissue as compared to a non-CNS tissue, wherein the non-CNS tissue consists collectively of liver, skeletal muscle, heart, lung, spleen, lymph node, bone marrow, mammary gland, skin, adrenal gland, thyroid, colon, sciatic nerve, and spinal cord.

99. The engineered AAV VP capsid polypeptide of claim 96, wherein the higher tissue tropism is a 1.0005-fold to about a 1000-fold increased accumulation in the CNS tissue as compared to a non-CNS tissue.

100. The engineered AAV VP capsid polypeptide of claim 99, wherein the higher tissue tropism is at least about a 1.0005-fold, at least about a two-fold, at least about a three-fold, at least about a four-fold, at least about a five-fold, at least about a ten-fold, at least about a twenty-fold, at least about a 50-fold, at least about a 75-fold, at least about a 100-fold, or at least about a 1000-fold increased accumulation in the CNS tissue as compared to a non-CNS tissue.

101. The engineered AAV VP capsid polypeptide of claim 93, wherein the mutation is a substitution.

102. The engineered AAV VP capsid polypeptide of claim 93, wherein the engineered AAV VP capsid polypeptide is an engineered AAV5 VP capsid polypeptide.

103. A recombinant AAV virion (rAAV), comprising:
the engineered AAV VP capsid polypeptide claim 93 assembled into a capsid; and
a payload,
wherein the engineered AAV VP capsid polypeptide encapsidates the payload.

104. The rAAV of claim 103, wherein the payload comprises a therapeutic polynucleotide, optionally wherein the therapeutic polynucleotide encodes a transgene or a genome modifying entity.

105. The rAAV of claim 103, wherein the therapeutic polynucleotide encodes a guide RNA, a tRNA, a suppressor tRNA, a siRNA, a miRNA, an mRNA, a shRNA, a circular RNA, or an antisense oligonucleotide (ASO), a ribozyme, a DNAzyme, an aptamer, or any combination thereof.

106. A method of treatment, comprising administering a therapeutically effective amount of the rAAV virion of claim 103 to a subject in need thereof, wherein the subject has a disease.

107. The method of claim 106, wherein the disease is a neurological condition selected from AADC deficiency, Alzheimer's Disease, tauopathies, synucleinopathies, Batten Disease, MPS-IIIB, frontotemporal dementia with GRN mutations (FTD-GRN), Parkinson's Disease with GBA1 mutations (PD-GBA), neuronpathic Gaucher's Disease, Gaucher Disease Type 2, Canavan Disease, Parkinson's Disease, Tay-Sachs Disease, Huntington's Disease, Protocki-Lupski Syndrome, amyotrophic lateral sclerosis, down syndrome, Sanfilippo Disease Type A, Sanfilippo Disease Type B, or Rett syndrome.
